(19) United States
(12) Patent Application Publication
(10) Pub. No.: US 2012/0034631 A1
(43) Pub. Date: Feb. 9, 2012

(54) ANTI-LYSOPHOSPHOLIPID ANTIBODY DESIGN USING ANTIBODY STRUCTURES

(76) Inventor: Jonathan Michael WOJCIAK, Encinitas, CA (US)

(21) Appl. No.: 13/153,435

(22) Filed: Jun. 5, 2011

Related U.S. Application Data
(60) Provisional application No. 61/353,915, filed on Jun. 11, 2010.
(60) Provisional application No. 61/363,219, filed on Jul. 10, 2010.
(60) Provisional application No. 61/433,166, filed on Jan. 14, 2011.

(51) Int. Cl. C07K 16/18; C07K 1/107; G06G 7/60; G01N 33/92

(52) U.S. Cl. 435/7.93; 530/389.8; 530/388.2; 530/387.3; 435/7.92; 436/501; 703/11

(57) ABSTRACT

The present invention provides crystalline forms of an anti-LPA antibody or fragment thereof, which may further comprise a lipid ligand of said antibody and/or salts, metals, or co-factors. Methods for making such crystals and co-crystals are provided, as are methods of using structural information in antibody design or optimization. Methods for designing a humanized antibody to a lipid are provided. These methods may be performed in silico and may be intended to enhance binding affinity of an antibody to its original target lipid, and/or to alter binding specificity. Optimized variant anti-LPA antibodies are also provided.

### GRANT SUPPORT

The subject matter of this application was supported at least in part by Small Business Innovation Research (SBIR) grant no. 1 R43 GM 088956-01. The U.S. Government may have certain rights herein.

### BACKGROUND OF THE INVENTION

1. Field of the Invention

The present invention relates to crystalline forms of anti-lipid antibodies, methods of making them, and methods of using data derived therefrom in antibody design and optimization. Methods for designing antibodies or antibody fragments are provided, wherein the antibody target is a lipid, such as a bioactive lipid.

The following description includes information that may be useful in understanding the present invention. It is not an admission that any of the information provided herein, or any publication specifically or implicitly referenced herein, is prior art, or even particularly relevant, to the presently claimed invention.

2. Background

Bioactive Signaling Lipids

Lipids and their derivatives are now recognized as important targets for medical research, not as just simple structural elements in cell membranes or as a source of energy for β-oxidation, glycolysis or other metabolic processes. In particular, certain bioactive lipids function as signaling mediators important in animal and human disease. Although most of the lipids of the plasma membrane play an exclusively structural role, a small proportion of them are involved in relaying extracellular stimuli into cells. “Lipid signaling” refers to any of a number of cellular signal transduction pathways that use cell membrane lipids as second messengers, as well as referring to direct interaction of a lipid signaling molecule with its own specific receptor. Lipid signaling pathways are activated by a variety of extracellular stimuli, ranging from growth factors to inflammatory cytokines, and regulate cell fate decisions such as apoptosis, differentiation and proliferation. Research into bioactive lipid signaling is an area of intense scientific investigation as more and more bioactive lipids are identified and their actions characterized.

Examples of bioactive lipids include the eicosanoids (including the cannabinoids, leukotrienes, prostaglandins, lipoxins, epoxyeicosatrienoic acids, and isoeicosanoids) such as the hydroxyeicosatetraenoic acids (HETEs, including 5-HETE, 12-HETE, 15-HETE and 20-HETE), non-eicosanoid cannabinoid mediators, phospholipids and their derivatives such as phosphatidic acid (PA) and phosphatidylglycerol (PG), platelet activating factor (PAF) and cardiolipins as well as lysophospholipids such as lysophosphatidyl choline (LPC) and various lysophosphatidic acids (LPA). Bioactive signaling lipid mediators also include the sphingolipids such as sphingomyelin, ceramide, ceramide-1-phosphate, sphingosine, sphingosylphosphoryl choline, sphinganine, sphinganine-1-phosphate (Dihydro-S1P) and sphingosine-1-phosphate. Sphingolipids and their derivatives represent a group of extracellular and intracellular signaling molecules with pleiotropic effects on important cellular processes. Other examples of bioactive signaling lipids include phosphatidylserine (PS), phosphatidylinositol (PI), phosphatidylethanolamine (PEA), diacylglyceride (DG), sulfatides, gangliosides, and cerebrosides.

Sphingolipids are a unique class of lipids that were named, due to their initially mysterious nature, after the Sphinx. Sphingolipids were initially characterized as primary structural components of cell membranes, but recent studies indicate that sphingolipids also serve as cellular signaling and regulatory molecules (Hannun, et al., Adv. Lipid Res. 25:27-41, 1993; Speigel, et al., FASEB J. 10:1388-1397, 1996; Igarashi, J. Biochem 122:1080-1087, 1997; Hla, T. (2004). Semin Cell Dev Biol, 15, 513-2; Gardell, S. E., Dubin, A. E. & Chun, J. (2006). Trends Mol Med, 12, 65-75). Sphingolipids are primary structural components of cell membranes that also serve as cellular signaling and regulatory molecules (Hannun and Bell, Adv. Lipid Res. 25: 27-41, 1993; Igarashi, J. Biochem 122: 1080-1087, 1997). The sphingolipid signaling mediators, ceramide (CER), sphingosine (SPH) and sphingosine-1-phosphate (S1P), have been most widely studied and have recently been appreciated for their roles in the cardiovascular system, angiogenesis and tumor biology (Claus, et al., Curr Drug Targets 1: 185-205, 2000; Levade, et al., Circ. Res. 89: 957-968, 2001; Wang, et al., J. Biol. Chem. 274: 35343-50, 1999; Wascholowski and Giannis, Drug News Perspect. 14: 581-90, 2001; Spiegel, S. & Milstien, S. (2003). Sphingosine-1-phosphate: an enigmatic signaling lipid. Nat Rev Mol Cell Biol, 4, 397-407).

For a review of sphingolipid metabolism, see Liu, et al., Crit. Rev. Clin. Lab. Sci. 36:511-573, 1999. For reviews of the sphingomyelin signaling pathway, see Hannun, et al., Adv. Lipid Res. 25:27-41, 1993; Liu, et al., Crit. Rev. Clin. Lab. Sci. 36:511-573, 1999; Igarashi, J. Biochem. 122:1080-1087, 1997; Oral, et al., J. Biol. Chem. 272:4836-4842, 1997; and Spiegel et al., Biochemistry (Moscow) 63:69-83, 1998.

Sphingosine-1-Phosphate (SIP)

S1P is a mediator of cell proliferation and protects from apoptosis through the activation of survival pathways (Maceyka, et al. (2002), BBA, vol. 1585): 192-201, and Spiegel, et al. (2003), Nature Reviews Molecular Cell Biology, vol. 4: 397-407). It has been proposed that the balance between CER/SPH levels and S1P provides a rheostat mechanism that decides whether a cell is directed into the death pathway or is protected from apoptosis. The key regulatory enzyme of the rheostat mechanism is sphingosine kinase (SPHK) whose role is to convert the death-promoting bioactive signaling lipids (CER/SPH) into the growth-promoting S1P. S1P has two fates: S1P can be degraded by S1P lyase, an enzyme that cleaves S1P to phosphoethanolamine and hexadecanal, or, less common, hydrolyzed by S1P phosphatase to SPH.

The pleiotropic biological activities of S1P are mediated via a family of G protein-coupled receptors (GPCRs) originally known as Endothelial Differentiation Genes (EDG). Five GPCRs have been identified as high-affinity S1P receptors (S1PRs): S1P₁/EDG-1, S1P₂/EDG-5, S1P₃/EDG-3, S1P₄/EDG-6, and S1P₅/EDG-8 only identified as late as 1998 (Lee, et al., 1998). Many responses evoked by S1P are coupled to different heterotrimeric G proteins (G_{q-}, Gᵢ, G₁₂₋₁₃) and the small GTPases of the Rho family (Gardell, et al., 2006).

In the adult, S1P is released from platelets (Murata et al., 2000) and mast cells to create a local pulse of free S1P (sufficient enough to exceed the Ka of the S1PRs) for promoting wound healing and participating in the inflammatory response. Under normal conditions, the total S1P in the plasma is quite high (300-500 nM); however, it has been hypothesized that most of the S1P may be ‘buffered’ by serum proteins, particularly lipoproteins (e.g., HDL>LDL>VLDL) and albumin, so that the bio-available S1P (or the free fraction of S1P) is not sufficient to appreciably activate S1PRs (Murata et al., 2000). If this were not the case, inappropriate angiogenesis and inflammation would result. Intracellular actions of S1P have also been suggested (see, e.g., Spiegel S, Kolesnick R (2002), Leukemia, vol. 16: 1596-602; Suomalainen, et al (2005), Am J Pathol, vol. 166: 773-81).

Widespread expression of the cell surface S1P receptors allows S1P to influence a diverse spectrum of cellular responses, including proliferation, adhesion, contraction, motility, morphogenesis, differentiation, and survival. This spectrum of response appears to depend upon the overlapping or distinct expression patterns of the S1P receptors within the cell and tissue systems. In addition, crosstalk between S1P and growth factor signaling pathways, including platelet-derived growth factor (PDGF), vascular endothelial growth factor (VEGF), and basic fibroblastic growth factor (bFGF), have recently been demonstrated (see, e.g., Baudhuin, et al. (2004), FASEB J, vol. 18: 341-3). The regulation of various cellular processes involving S1P has particular impact on neuronal signaling, vascular tone, wound healing, immune cell trafficking, reproduction, and cardiovascular function, among others. Alterations of endogenous levels of S1P within these systems can have detrimental effects, eliciting several pathophysiological conditions, including cancer, inflammation, angiogenesis, heart disease, asthma, and autoimmune diseases.

A recent novel approach to the treatment of various diseases and disorders, including cardiovascular diseases, cerebrovascular diseases, and various cancers, involves reducing levels of biologically available S1P, either alone or in combination with other treatments. While sphingolipid-based treatment strategies that target key enzymes of the sphingolipid metabolic pathway, such as SPHK, have been proposed, interference with the lipid mediator S1P itself has not until recently been emphasized, largely because of difficulties in directly mitigating this lipid target, in particular because of the difficulty first in raising and then in detecting antibodies against the S1P target.

Recently, the generation of antibodies specific for S1P has been described. See, e.g., commonly owned, U.S. patent application Serial No. 20070148168; WO2007/053447. Such antibodies, which can, for example, selectively adsorb S1P from serum, act as molecular sponges to neutralize extracellular S1P. See also commonly owned U.S. Pat. Nos. 6,881,546 and 6,858,383 and U.S. patent application Ser. No. 10/029,372. SPHINGOMAB™, the murine monoclonal antibody (mAb) developed by Lpath, Inc. and described in certain patents or patent applications listed above, has been shown to be effective in models of human disease. In some situations, a humanized antibody may be preferable to a murine antibody, particularly for therapeutic uses in humans, where human-anti-mouse antibody (HAMA) response may occur. Such a response may reduce the effectiveness of the antibody by neutralizing the binding activity and/or by rapidly clearing the antibody from circulation in the body. The HAMA response can also cause toxicities with subsequent administrations of mouse antibodies.

A first-in-class humanized anti-S1P antibody (Sonepcizumab, LT1009) has now been developed and is described herein. This antibody is expected to have all the advantages of the murine mAb in terms of efficacy in binding S1P, neutralizing S1P and modulating disease states related to S1P, but with none of the potential disadvantages of the murine mAb when used in a human context. As described in the examples hereinbelow, this humanized antibody has in fact shown activity greater than that of the parent (murine) antibody in animal models of disease. Sonepcizumab is currently in clinical trials for cancer and age-related macular degeneration.

A Sonepcizumab Fab fragment has been crystallized in complex with S1P, and the structure of the complex has been determined. Wojciak, J. et al., Proc Natl Acad Sci USA. 2009 Oct. 20; 106(42):17717-22. Epub 2009 Oct. 7.

Lysolipids

Lysolipids are low molecular weight lipids that contain a polar head group and a single hydrocarbon backbone, due to the absence of an acyl group at one or both possible positions of acylation. Relative to the polar head group at sn-3, the hydrocarbon chain can be at the sn-2 and/or sn-1 position(s) (the term “lyso,” which originally related to hemolysis, has been redefined by IUPAC to refer to deacylation). See “Nomenclature of Lipids, www.chem.qmul.ac.uk/iupac/lipid/lip1n2.html. These lipids are representative of signaling, bioactive lipids, and their biologic and medical importance highlight what can be achieved by targeting lipid signaling molecules for therapeutic, diagnostic/prognostic, or research purposes (Gardell, et al. (2006), Trends in Molecular Medicine, vol 12: 65-75). Two particular examples of medically important lysolipids are LPA (glycerol backbone) and S1P (sphingoid backbone). Other lysolipids include sphingosine, lysophosphatidylcholine (LPC), sphingosylphosphorylcholine (lysosphingomyelin), ceramide, ceramide-1-phosphate, sphinganine (dihydrosphingosine), dihydrosphingosine-1-phosphate and N-acetyl-ceramide-1-phosphate. In contrast, the plasmalogens, which contain an O-alkyl (—O—CH₂—) or O-alkenyl ether at the C-1 (sn1) and an acyl at C-2, are excluded from the lysolipid genus. The structures of selected LPAs, S1P, and dihydro S1P are presented below.

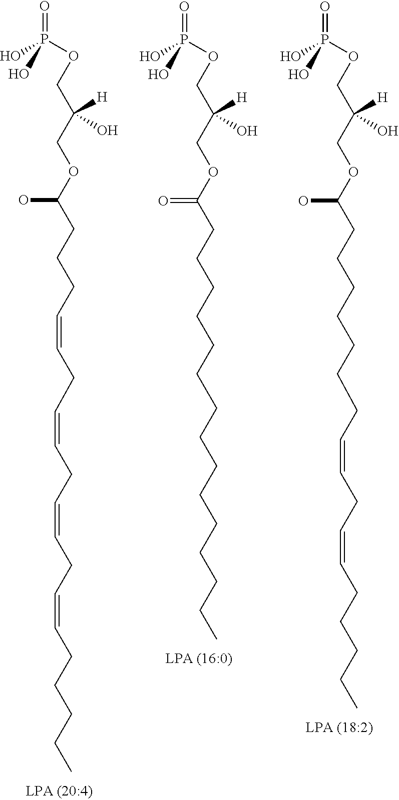

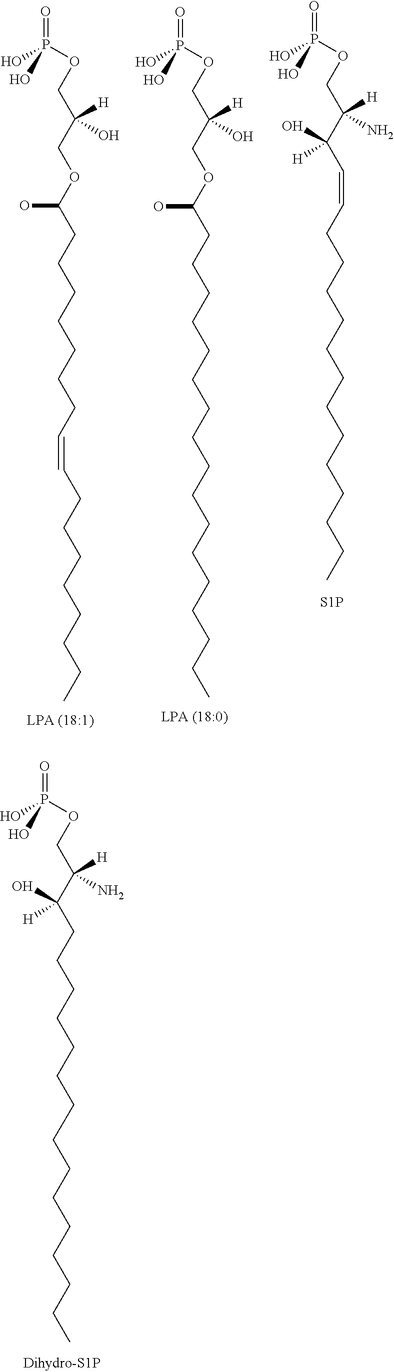

LPA is not a single molecular entity but a collection of endogenous structural variants with fatty acids of varied lengths and degrees of saturation (Fujiwara, et al. (2005), J Biol Chem, vol. 280: 35038-35050). The structural backbone of the LPAs is derived from glycerol-based phospholipids such as phosphatidylcholine (PC) or phosphatidic acid (PA). In the case of lysosphingolipids such as S1P, the fatty acid of the ceramide backbone at sn-2 is missing. The structural backbone of S1P, dihydro S1P (DHS1P) and sphingosylphosphorylcholine (SPC) is based on sphingosine, which is derived from sphingomyelin.

LPA and S1P regulate various cellular signaling pathways by binding to the same class of multiple transmembrane domain G protein-coupled (GPCR) receptors (Chun J, Rosen H (2006), Current Pharm Des, vol. 12: 161-171, and Moolenaar, W H (1999), Experimental Cell Research, vol. 253: 230-238). The S1P receptors are designated as S1P₁, S1P₂, S1P₃, S1P₄ and S1P₅ (formerly EDG-1, EDG-5/AGR16, EDG-3, EDG-6 and EDG-8) and the LPA receptors designated as LPA₁, LPA₂, LPA₃ (formerly, EDG-2, EDG-4, and EDG-7). A fourth LPA receptor of this family has been identified for LPA (LPA₄), and other putative receptors for these lysophospholipids have also been reported.

### Lysophosphatic Acids (LPA)

LPAs have long been known as precursors of phospholipid biosynthesis in both eukaryotic and prokaryotic cells, but LPAs have emerged only recently as signaling molecules that are rapidly produced and released by activated cells, notably platelets, to influence target cells by acting on specific cell-surface receptor (see, e.g., Moolenaar, of al. (2004), BioEssays, vol. 26: 870-881, and van Leewen of al. (2003), Biochem Soc Trans, vol 31: 1209-1212). Besides being synthesized and processed to more complex phospholipids in the endoplasmic reticulum, LPA can be generated through the hydrolysis of pre-existing phospholipids following cell activation; for example, the sn-2 position is commonly missing a fatty acid residue due to deacylation, leaving only the sn-1 hydroxyl esterified to a fatty acid. Moreover, a key enzyme in the production of LPA, autotoxin (lysoPLD/NPP2), may be the product of an oncogene, as many tumor types up-regulate autotoxin (Brindley, D. (2004), J Cell Biochem, vol. 92: 900-12). The concentrations of LPA in human plasma and serum have been reported, including determinations made using a sensitive and specific LC/MS procedure (Baker, et al. (2001), Anal Biochem, vol 292: 287-295). For example, in freshly prepared human serum allowed to sit at 25° C. for one hour, LPA concentrations have been estimated to be approximately 1.2 μM, with the LPA analogs 16:0, 18:1, 18:2, and 20:4 being the predominant species. Similarly, in freshly prepared human plasma allowed to sit at 25° C. for one hour, LPA concentrations have been estimated to be approximately 0.7 μM, with 18:1 and 18:2 LPA being the predominant species.

LPA influences a wide range of biological responses, ranging from induction of cell proliferation, stimulation of cell migration and neurite retraction, gap junction closure, and even slime mold chemotaxis (Goetzl, et al. (2002), Scientific World Journal, vol. 2: 324-338). The body of knowledge about the biology of LPA continues to grow as more and more cellular systems are tested for LPA responsiveness. For instance, it is now known that, in addition to stimulating cell growth and proliferation, LPA promote cellular tension and cell-surface fibronectin binding, which are important events in wound repair and regeneration (Moolenaar, et al. (2004), BioEssays, vol. 26: 870-881). Recently, anti-apoptotic activity has also been ascribed to LPA, and it has recently been reported that peroxisome proliferation receptor gamma is a receptor/target for LPA (Simon, et al. (2005), J Biol Chem, vol. 280: 14656-14662). LPA is now recognized as a key signaling molecule involved in the etiology of cancer. Murph, M and Mills, G B (2007) Expert Rev. Mol. Med. 9:1-18.

LPA has proven to be a difficult target for antibody production, although there has been a report in the scientific literature of the production of polyclonal murine antibodies against LPA (Chen et al. (2000) Med Chem Lett, vol 10: 1691-3).

Lpath has recently humanized a monoclonal antibody against LPA, disclosed in US Patent application US20080145360 (attorney docket no. LPT-3100-UT4). The humanized anti-LPA antibody, LT3015, exhibits picomolar binding affinity as demonstrated using surface plasmon resonance and is highly specific for LPA.

Structure and Design of Monoclonal Antibodies

Soluble antibodies of the Immunoglobin G (IgG) class consist of a pair of heavy and light chains that are held together by intra- and interchain disulfide bonds to generate the characteristic Y-shaped structure (FIG. 1). In terms of protein tertiary structure, antibodies consist entirely of the immunoglobin domain—a fold that is common to many effector molecules of the immune system. Heavy chains begin with one variable domain (Vh) followed by three constant domains (Ch1-3) while kappa light chains consist of one variable domain (Vk) followed by one constant domain (Ck). Epitope binding specificity results from variability within the amino-terminal Vh and Vk domains, particularly within six loops (CDR H1, H2, H3, L1, L2 and L3) also known as hypervariable regions.

Treatment of purified whole IgG preparations with the protease papain separates a Fab fragment consisting of both variable domains and the Ck and constant domains from the Fc domain, which contains a pair of Ch2 and Ch3 domains. The Fab fragment retains one entire variable region and, therefore, serves as a useful tool for biochemical characterization of a 1:1 interaction between the antibody and epitope. Furthermore, because it lacks the flexibility and, generally, the glycosylation inherent in native purified whole IgG, the Fab fragment is generally an excellent platform for structural studies via single crystal x-ray diffraction.

Currently, there are over 20 therapeutic antibodies on the market. It is the fastest growing segment of therapeutics largely because humanized mAbs have a high safety profile. The huge success of antibody molecular sponges like Avastin, Lucentis, Humira and Remicade have demonstrated that the use of antibody therapeutics in this mode can also be effective in the treatment of cancer, AMD, inflammatory and autoimmune disorders by neutralizing the target (in the cited cases, protein growth factors) in the extracellular space and depriving receptors of their ligand.

Lpath's ImmuneY2™ technology allows generation of monoclonal antibodies (mAb) against extracellular lipid signaling mediators. Lpath has developed a first-in-class therapeutic agent, a humanized monoclonal antibody Sonepcizumab™ (LT1009; the names Sonepcizumab and LT1009 are herein used interchangeably), which was derived from the murine form of the antibody, Sphingomab™. Sonepcizumab neutralizes the bioactive lipid signaling mediator, sphingosine-1-phosphate (S1P). S1P contributes to disease in cancer, multiple sclerosis, inflammatory disease and ocular diseases that involve dysregulated angiogenesis. A systemic formulation of Sonepcizumab, ASONEP™, is currently in Phase 1 trials for cancer while an ocular formulation of the same mAb, iSONEP™, is in Phase 1 clinical trials for Age-related Macular Degeneration (AMD). Lpath has also recently developed the humanized mAb Lpathomab™ (LT3015; the names Lpathomab and LT3015 are herein used interchangeably), a mAb against the bioactive lipid mediator, lysophosphatidic acid (LPA). In addition to regulating physiological responses such as cell adhesion, motility, cytoskeletal changes, proliferation, angiogenesis, neurite retraction, and cell survival, LPA has been implicated in the pathogenesis and progression of severe diseases including cancer, fibrosis, neuropathic pain, and inflammatory diseases.

3. Definitions

Before describing the instant invention in detail, several terms used in the context of the present invention will be defined. In addition to these terms, others are defined elsewhere in the specification, as necessary. Unless otherwise expressly defined herein, terms of art used in this specification will have their art-recognized meanings.

The term “antibody” (“Ab”) or “immunoglobulin” (Ig) refers to any form of a peptide, polypeptide derived from, modeled after or encoded by, an immunoglobulin gene, or fragment thereof, that is capable of binding an antigen or epitope. See, e.g., IMMUNOBIOLOGY, Fifth Edition, C. A. Janeway, P. Travers, M., Walport, M. J. Shlomchiked., ed. Garland Publishing (2001). The term “antibody” is used herein in the broadest sense, and encompasses monoclonal, polyclonal or multispecific antibodies, minibodies, heteroconjugates, diabodies, triabodies, chimeric, antibodies, synthetic antibodies, antibody fragments, and binding agents that employ the complementarity determining regions (CDRs) of the parent antibody, or variants thereof that retain antigen binding activity. Antibodies are defined herein as retaining at least one desired activity of the parent antibody. Desired activities can include the ability to bind the antigen specifically, the ability to inhibit proleration in vitro, the ability to inhibit angiogenesis in vivo, and the ability to alter cytokine profile(s) in vitro.

Native antibodies (native immunoglobulins) are usually heterotetrameric glycoproteins of about 150,000 Daltons, typically composed of two identical light (L) chains and two identical heavy (H) chains. The heavy chain is approximately 50 kD in size, and the light chain is approximately 25 kDa. Each light chain is typically linked to a heavy chain by one covalent disulfide bond, while the number of disulfide linkages varies among the heavy chains of different immunoglobulin isotypes. Each heavy and light chain also has regularly spaced intrachain disulfide bridges. Each heavy chain has at one end a variable domain (V_{H}) followed by a number of constant domains. Each light chain has a variable domain at one end (V_{L}) and a constant domain at its other end; the constant domain of the light chain is aligned with the first constant domain of the heavy chain, and the light-chain variable domain is aligned with the variable domain of the heavy chain. Particular amino acid residues are believed to form an interface between the light- and heavy-chain variable domains.

The light chains of antibodies (immunoglobulins) from any vertebrate species can be assigned to one of two clearly distinct types, called kappa (κ) and lambda (λ), based on the amino acid sequences of their constant domains. The ratio of the two types of light chain varies from species to species. As a way of example, the average κ to λ ratio is 20:1 in mice, whereas in humans it is 2:1 and in cattle it is 1:20.

Depending on the amino acid sequence of the constant domain of their heavy chains, immunoglobulins can be assigned to different classes. There are five major classes of immunoglobulins: IgA, IgD, IgE, IgG, and IgM, and several of these may be further divided into subclasses (isotypes), e.g., IgG1, IgG2, IgG3, IgG4, IgA, and IgA2. The heavy-chain constant domains that correspond to the different classes of immunoglobulins are called alpha, delta, epsilon, gamma, and mu, respectively. The subunit structures and three-dimensional configurations of different classes of immunoglobulins are well known.

An “antibody derivative” is an immune-derived moiety, i.e., a molecule that is derived from an antibody. This includes any antibody (Ab) or immunoglobulin (Ig), and refers to any form of a peptide, polypeptide derived from, modeled after or encoded by, an immunoglobulin gene, or a fragment of such peptide or polypeptide that is capable of binding an antigen or epitope. This comprehends, for example, antibody variants, antibody fragments, chimeric antibodies, humanized antibodies, multivalent antibodies, antibody conjugates and the like, which retain a desired level of binding activity for antigen.

As used herein, “antibody fragment” refers to a portion of an intact antibody that includes the antigen binding site or variable regions of an intact antibody, wherein the portion can be free of the constant heavy chain domains (e.g., CH2, CH3, and CH4) of the Fc region of the intact antibody. Alternatively, portions of the constant heavy chain domains (e.g., CH2, CH3, and CH4) can be included in the “antibody fragment”. Antibody fragments retain antigen-binding and include Fab, Fab′, F(ab′)₂, Fd, and Fv fragments; diabodies; triabodies; single-chain antibody molecules (sc-Fv); minibodies, nanobodies, and multispecific antibodies formed from antibody fragments. Papain digestion of antibodies produces two identical antigen-binding fragments, called “Fab” fragments, each with a single antigen-binding site, and a residual “Fc” fragment, whose name reflects its ability to crystallize readily. Pepsin treatment yields an F(ab′)₂ fragment that has two antigen-combining sites and is still capable of cross-linking antigen. By way of example, a Fab fragment also contains the constant domain of a light chain and the first constant domain (CH1) of a heavy chain. “Fv” is the minimum antibody fragment that contains a complete antigen-recognition and -binding site. This region consists of a dimer of one heavy chain and one light chain variable domain in tight, non-covalent association. It is in this configuration that the three hypervariable regions of each variable domain interact to define an antigen-binding site on the surface of the V_{H}-V_{L} dimer. Collectively, the six hypervariable regions confer antigen-binding specificity to the antibody. However, even a single variable domain (or half of an Fv comprising only three hypervariable regions specific for an antigen) has the ability to recognize and bind antigen, although at a lower affinity than the entire binding site. “Single-chain Fv” or “sFv” antibody fragments comprise the V_{H} and V_{L} domains of antibody, wherein these domains are present in a single polypeptide chain. Generally, the Fv polypeptide further comprises a polypeptide linker between the V_{H} and V_{L} domains that enables the sFv to form the desired structure for antigen binding. For a review of sFv, see Pluckthun in The Pharmacology of Monoclonal Antibodies, vol. 113, Rosenburg and Moore eds. Springer-Verlag, New York, pp. 269-315 (1994).

The Fab fragment also contains the constant domain of the light chain and the first constant domain (CH1) of the heavy chain. Fab′ fragments differ from Fab fragments by the addition of a few residues at the carboxyl terminus of the heavy chain CH1 domain including one or more cysteine(s) from the antibody hinge region. Fab′-SH is the designation herein for Fab′ in which the cysteine residue(s) of the constant domains bear a free thiol group. F(ab′)₂ antibody fragments originally were produced as pairs of Fab′ fragments which have hinge cysteines between them. Other chemical couplings of antibody fragments are also known.

An “antibody variant” refers herein to a molecule which differs in amino acid sequence from the amino acid sequence of a native or parent antibody that is directed to the same antigen by virtue of addition, deletion and/or substitution of one or more amino acid residue(s) in the antibody sequence and which retains at least one desired activity of the parent anti-binding antibody. Desired activities can include the ability to bind the antigen specifically, the ability to inhibit proliferation in vitro, the ability to inhibit angiogenesis in vivo, and the ability to alter cytokine profile in vitro. The amino acid change(s) in an antibody variant may be within a variable region or a constant region of a light chain and/or a heavy chain, including in the Fc region, the Fab region, the CH₁ domain, the CH₂ domain, the CH₃ domain, and the hinge region. In one embodiment, the variant comprises one or more amino acid substitution(s) in one or more hypervariable region(s) of the parent antibody. For example, the variant may comprise at least one, e.g. from about one to about ten, and preferably from about two to about five, substitutions in one or more hypervariable regions of the parent antibody. Ordinarily, the variant will have an amino acid sequence having at least 50% amino acid sequence identity with the parent antibody heavy or light chain variable domain sequences, more preferably at least 65%, more preferably at 80%, more preferably at least 85%, more preferably at least 90%, and most preferably at least 95%. Identity or homology with respect to this sequence is defined herein as the percentage of amino acid residues in the candidate sequence that are identical with the parent antibody residues, after aligning the sequences and introducing gaps, if necessary, to achieve the maximum percent sequence identity. None of N-terminal, C-terminal, or internal extensions, deletions, or insertions into the antibody sequence shall be construed as affecting sequence identity or homology. The variant retains the ability to bind LPA and preferably has desired activities which are superior to those of the parent antibody. For example, the variant may have a stronger binding affinity, enhanced ability to reduce angiogenesis and/or halt tumor progression. To analyze such desired properties (for example les immunogenic, longer half-life, enhanced stability, enhanced potency), one should compare a Fab form of the variant to a Fab form of the parent antibody or a full length form of the variant to a full length form of the parent antibody, for example, since it has been found that the format of the antibody impacts its activity in the biological activity assays disclosed herein. The variant antibody of particular interest herein can be one which displays at least about 10 fold, preferably at least about % 5, 25, 59, or more of at least one desired activity. The preferred variant is one that has superior biophysical properties as measured in vitro or superior activities biological as measured in vitro or in vivo when compared to the parent antibody.

An “anti-LPA agent” refers to any therapeutic agent that binds LPA, and includes antibodies, antibody variants, antibody-derived molecules or non-antibody-derived moieties that bind LPA and its variants.

An “anti-LPA antibody” or an “immune-derived moiety reactive against LPA” refers to any antibody or antibody-derived molecule that binds LPA. As will be understood from these definitions, antibodies or immune-derived moieties may be polyclonal or monoclonal and may be generated through a variety of means, and/or may be isolated from an animal, including a human subject.

An “anti-S1P agent” refers to any therapeutic agent that binds S1P, and includes antibodies, antibody variants, antibody-derived molecules or non-antibody-derived moieties that bind LPA and its variants.

An “anti-S1P antibody” or an “immune-derived moiety reactive against S1P” refers to any antibody or antibody-derived molecule that binds S1P. As will be understood from these definitions, antibodies or immune-derived moieties may be polyclonal or monoclonal and may be generated through a variety of means, and/or may be isolated from an animal, including a human subject.

A “bioactive lipid” refers to a lipid signaling molecule. Bioactive lipids are distinguished from structural lipids (e.g., membrane-bound phospholipids) in that they mediate extracellular and/or intracellular signaling and thus are involved in controlling the function of many types of cells by modulating differentiation, migration, proliferation, secretion, survival, and other processes. In vivo, bioactive lipids can be found in extracellular fluids, where they can be complexed with other molecules, for example serum proteins such as albumin and lipoproteins, or in “free” form, i.e., not complexed with another molecule species. As extracellular mediators, some bioactive lipids alter cell signaling by activating membrane-bound ion channels or GPCRs or enzymes or factors that, in turn, activate complex signaling systems that result in changes in cell function or survival. As intracellular mediators, bioactive lipids can exert their actions by directly interacting with intracellular components such as enzymes, ion channels or structural elements such as actin.

Examples of bioactive lipids include sphingolipids such as ceramide, ceramide-1-phosphate (C1P), sphingosine, sphinganine, sphingosylphosphorylcholine (SPC) and sphingosine-1-phosphate (S1P). Sphingolipids and their derivatives and metabolites are characterized by a sphingoid backbone (derived from sphingomyelin). Sphingolipids and their derivatives and metabolites represent a group of extracellular and intracellular signaling molecules with pleiotropic effects on important cellular processes. They include sulfatides, gangliosides and cerebrosides. Other bioactive lipids are characterized by a glycerol-based backbone; for example, lysophospholipids such as lysophosphatidyl choline (LPC) and various lysophosphatidic acids (LPA), as well as phosphatidylinositol (PI), phosphatidylethanolamine (PEA), phosphatidic acid, platelet activating factor (PAF), cardiolipin, phosphatidylglycerol (PG) and diacylglyceride (DG). Yet other bioactive lipids are derived from arachidonic acid; these include the eicosanoids (including the eicosanoid metabolites such as the HETEs, cannabinoids, leukotrienes, prostaglandins, lipoxins, epoxyeicosatrienoic acids, and isoeicosanoids), non-eicosanoid cannabinoid mediators. Other bioactive lipids, including other phospholipids and their derivatives, may also be used according to the instant invention.

In some embodiments of the invention it may be preferable to target glycerol-based bioactive lipids (those having a glycerol-derived backbone, such as the LPAs) for antibody production, as opposed to sphingosine-based bioactive lipids (those having a sphingoid backbone, such as sphingosine and S1P). In other embodiments it may be desired to target arachidonic acid-derived bioactive lipids for antibody generation, and in other embodiments arachidonic acid-derived and glycerol-derived bioactive lipids but not sphingoid-derived bioactive lipids are preferred. Together the arachidonic acid-derived and glycerol-derived bioactive lipids may be referred to in the context of this invention as “non-sphingoid bioactive lipids.”

Specifically excluded from the class of bioactive lipids according to the invention are phosphatidylcholine and phosphatidylserine, as well as their metabolites and derivatives that function primarily as structural members of the inner and/or outer leaflet of cellular membranes.

The term “biologically active,” in the context of an antibody or antibody fragment or variant, refers to an antibody or antibody fragment or antibody variant that is capable of binding the desired epitope and in some ways exerting a biologic effect. Biological effects include, but are not limited to, the modulation of a growth signal, the modulation of an anti-apoptotic signal, the modulation of an apoptotic signal, the modulation of the effector function cascade, and modulation of other ligand interactions.

A “biomarker” is a specific biochemical in the body which has a particular molecular feature that makes it useful for measuring the progress of disease or the effects of treatment. For example, S1P is a biomarker for certain hyperproliferative and/or cardiovascular conditions.

The term “cardiotherapeutic agent” refers to an agent that is therapeutic to diseases and diseases caused by or associated with cardiac and myocardial diseases and disorders.

“Cardiovascular therapy” encompasses cardiac therapy (treatment of myocardial ischemia and/or heart failure) as well as the prevention and/or treatment of other diseases associated with the cardiovascular system, such as heart disease. The term “heart disease” encompasses any type of disease, disorder, trauma or surgical treatment that involves the heart or myocardial tissue. Of particular interest are conditions associated with tissue remodeling. The term “cardiotherapeutic agent” refers to an agent that is therapeutic to diseases and diseases caused by or associated with cardiac and myocardial diseases and disorders.

A “carrier” refers to a moiety adapted for conjugation to a hapten, thereby rendering the hapten immunogenic. A representative, non-limiting class of carriers is proteins, examples of which include albumin, keyhole limpet hemocyanin, hemaglutanin, tetanus, and diptheria toxoid. Other classes and examples of carriers suitable for use in accordance with the invention are known in the art. These, as well as later discovered or invented naturally occurring or synthetic carriers, can be adapted for application in accordance with the invention.

As used herein, the expressions “cell,” “cell line,” and “cell culture” are used interchangeably and all such designations include progeny. Thus, the words “transformants” and “transformed cells” include the primary subject cell and cultures derived there from without regard for the number of transfers. It is also understood that all progeny may not be precisely identical in DNA content, due to deliberate or inadvertent mutations. Mutant progeny that have the same function or biological activity as screened for in the originally transformed cell are included. Where distinct designations are intended, it will be clear from the context.

“Cerebrovascular therapy” refers to therapy directed to the prevention and/or treatment of diseases and disorders associated with cerebral ischemia and/or hypoxia. Of particular interest is cerebral ischemia and/or hypoxia resulting from global ischemia resulting from a heart disease, including without limitation heart failure.

The term “chemotherapeutic agent” means anti-cancer and other anti-hyperproliferative agents. Thus chemotherapeutic agents are a subset of therapeutic agents in general. Chemotherapeutic agents include, but are not limited to: DNA damaging agents and agents that inhibit DNA synthesis: anthracyclines (doxorubicin, donorubicin, epirubicin), alkylating agents (bendamustine, busulfan, carboplatin, carmustine, chlorambucil, cyclophosphamide, dacarbazine, hexamethylmelamine, ifosphamide, lomustine, mechlorethamine, melphalan, mitotane, mytomycin, pipobroman, procarbazine, streptozocin, thiotepa, and triethylenemelamine), platinum derivatives (cisplatin, carboplatin, cis diammine-dichloroplatinum), and topoisomerase inhibitors (Camptosar); anti-metabolites such as capecitabine, chlorodeoxyadenosine, cytarabine (and its activated form, ara-CMP), cytosine arabinoside, dacabazine, floxuridine, fludarabine, 5-fluorouracil, 5-DFUR, gemcitabine, hydroxyurea, 6-mercaptopurine, methotrexate, pentostatin, trimetrexate, 6-thioguanine); anti-angiogenics (bevacizumab, thalidomide, sunitinib, lenalidomide, TNP-470, 2-methoxyestradiol, ranibizumab, sorafenib, erlotinib, bortezomib, pegaptanib, endostatin); vascular disrupting agents (flavonoids/flavones, DMXAA, combretastatin derivatives such as CA4DP, ZD6126, AVE8062A, etc.); biologics such as antibodies (Herceptin, Avastin, Panorex, Rituxin, Zevalin, Mylotarg, Campath, Bexxar, Erbitux); endocrine therapy: aromatase inhibitors (4-hydroandrostendione, exemestane, aminoglutehimide, anastrazole, letozole), anti-estrogens (Tamoxifen, Toremifine, Raoxifene, Faslodex), steroids such as dexamethasone; immuno-modulators: cytokines such as IFN-beta and IL2), inhibitors to integrins, other adhesion proteins and matrix metalloproteinases); histone deacetylase inhibitors like suberoylanilide hydroxamic acid; inhibitors of signal transduction such as inhibitors of tyrosine kinases like imatinib (Gleevec); inhibitors of heat shock proteins like 17-N-allylamino-17-demethoxygeldanamycin; retinoids such as all trans retinoic acid; inhibitors of growth factor receptors or the growth factors themselves; anti-mitotic compounds and/or tubulin-depolymerizing agents such as the taxoids (paclitaxel, docetaxel, taxotere, BAY 59-8862), navelbine, vinblastine, vincristine, vindesine and vinorelbine; anti-inflammatories such as COX inhibitors and cell cycle regulators, e.g., check point regulators and telomerase inhibitors.

The term “chimeric” antibody (or immunoglobulin) refers to a molecule comprising a heavy and/or light chain which is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (Cabilly, et al., infra; Morrison et al., Proc. Natl. Acad. Sci. U.S.A., vol. 81:6851 (1984)).

The term “combination therapy” refers to a therapeutic regimen that involves the provision of at least two distinct therapies to achieve an indicated therapeutic effect. For example, a combination therapy may involve the administration of two or more chemically distinct active ingredients, for example, a fast-acting chemotherapeutic agent and an anti-lipid antibody, or two different antibodies. Alternatively, a combination therapy may involve the administration of an anti-lipid antibody together with the delivery of another treatment, such as radiation therapy and/or surgery. Further, a combination therapy may involve administration of an anti-lipid antibody together with one or more other biological agents (e.g., anti-VEGF, TGFβ, PDGF, or bFGF agent), chemotherapeutic agents and another treatment such as radiation and/or surgery. In the context of the administration of two or more chemically distinct active ingredients, it is understood that the active ingredients may be administered as part of the same composition or as different compositions. When administered as separate compositions, the compositions comprising the different active ingredients may be administered at the same or different times, by the same or different routes, using the same of different dosing regimens, all as the particular context requires and as determined by the attending physician. Similarly, when one or more anti-lipid antibody species, for example, an anti-LPA antibody, alone or in conjunction with one or more chemotherapeutic agents are combined with, for example, radiation and/or surgery, the drug(s) may be delivered before or after surgery or radiation treatment.

The term “constant domain” refers to the C-terminal region of an antibody heavy or light chain. Generally, the constant domains are not directly involved in the binding properties of an antibody molecule to an antigen, but exhibit various effector functions, such as participation of the antibody in antibody-dependent cellular toxicity. Here, “effector functions” refer to the different physiological effects of antibodies (e.g., opsonization, cell lysis, mast cell, basophil and eosinophil degranulation, and other processes) mediated by the recruitment of immune cells by the molecular interaction between the Fc domain and proteins of the immune system. The isotype of the heavy chain determines the functional properties of the antibody. Their distinctive functional properties are conferred by the carboxy-terminal portions of the heavy chains, where they are not associated with light chains.

The expression “control sequences” refers to DNA sequences necessary for the expression of an operably linked coding sequence in a particular host organism. The control sequences that are suitable for prokaryotes, for example, include a promoter, optionally an operator sequence, and a ribosome binding site. Eukaryotic cells are known to utilize promoters, polyadenylation signals, and enhancers.

A “derivatized bioactive lipid” is a bioactive lipid, e.g., LPA, which has a polar head group and at least one hydrocarbon chain, wherein a carbon atom within the hydrocarbon chain is derivatized with a pendant reactive group [e.g., a sulfhydryl (thiol) group, a carboxylic acid group, a cyano group, an ester, a hydroxy group, an alkene, an alkyne, an acid chloride group or a halogen atom] that may or may not be protected. This derivatization serves to activate the bioactive lipid for reaction with a molecule, e.g., for conjugation to a carrier.

A “derivatized bioactive lipid conjugate” refers to a derivatized bioactive lipid that is covalently conjugated to a carrier. The carrier may be a protein molecule or may be a moiety such as polyethylene glycol, colloidal gold, adjuvants or silicone beads. A derivatized bioactive lipid conjugate may be used as an immunogen for generating an antibody response according to the instant invention, and the same or a different bioactive lipid conjugate may be used as a detection reagent for detecting the antibody thus produced. In some embodiments the derivatized bioactive lipid conjugate is attached to a solid support when used for detection.

The term “diabodies” refers to small antibody fragments with two antigen-binding sites, which fragments comprise a heavy chain variable domain (V_{H}) connected to a light chain variable domain (V_{L}) in the same polypeptide chain (V_{H}-V_{L}). By using a linker that is too short to allow pairing between the two domains on the same chain, the domains are forced to pair with the complementary domains of another chain and create two antigen-binding sites. Diabodies are described more fully in, for example, EP 404,097; WO 93/11161; and Hollinger et al., Proc. Natl. Acad. Sci. USA 90:6444-6448 (1993).

“Effective concentration” refers to the absolute, relative, and/or available concentration and/or activity, for example of certain undesired bioactive lipids. In other words, the effective concentration of a bioactive lipid is the amount of lipid available, and able, to perform its biological function. In the present invention, an immune-derived moiety such as, for example, a monoclonal antibody directed to a bioactive lipid (such as, for example, C1P) is able to reduce the effective concentration of the lipid by binding to the lipid and rendering it unable to perform its biological function. In this example, the lipid itself is still present (it is not degraded by the antibody, in other words) but can no longer bind its receptor or other targets to cause a downstream effect, so “effective concentration” rather than absolute concentration is the appropriate measurement. Methods and assays exist for directly and/or indirectly measuring the effective concentration of bioactive lipids.

An “epitope” or “antigenic determinant” refers to that portion of an antigen that reacts with an antibody antigen-binding portion derived from an antibody.

The term “expression cassette” refers to a nucleotide molecule capable of affecting expression of a structural gene (i.e., a protein coding sequence, such as an antibody of the invention) in a host compatible with such sequences. Expression cassettes include at least a promoter operably linked with the polypeptide-coding sequence, and, optionally, with other sequences, e.g., transcription termination signals. Additional regulatory elements necessary or helpful in effecting expression may also be used, e.g., enhancers. Thus, expression cassettes include plasmids, expression vectors, recombinant viruses, any form of recombinant “naked DNA” vector, and the like.

A “fully human antibody” can refer to an antibody produced in a genetically engineered (i.e., transgenic) mouse (e.g. from Medarex) that, when presented with an immunogen, can produce a human antibody that does not necessarily require CDR grafting. These antibodies are fully human (100% human protein sequences) from animals such as mice in which the non-human antibody genes are suppressed and replaced with human antibody gene expression. The applicants believe that antibodies could be generated against bioactive lipids when presented to these genetically engineered mice or other animals who might be able to produce human frameworks for the relevant CDRs.

A “hapten” is a substance that is non-immunogenic but can react with an antibody or antigen-binding portion derived from an antibody. In other words, haptens have the property of antigenicity but not immunogenicity. A hapten is generally a small molecule that can, under most circumstances, elicit an immune response (i.e., act as an antigen) only when attached to a carrier, for example, a protein, polyethylene glycol (PEG), colloidal gold, silicone beads, or the like. The carrier may be one that also does not elicit an immune response by itself. A representative, non-limiting class of hapten molecules is proteins, examples of which include albumin, keyhole limpet hemocyanin, hemaglutanin, tetanus, and diphtheria toxoid. Other classes and examples of hapten molecules are known in the art. These, as well as later discovered or invented naturally occurring or synthetic haptens, can be adapted for application in accordance with the invention.

The term “heteroconjugate antibody” can refer to two covalently joined antibodies. Such antibodies can be prepared using known methods in synthetic protein chemistry, including using crosslinking agents. As used herein, the term “conjugate” refers to molecules formed by the covalent attachment of one or more antibody fragment(s) or binding moieties to one or more polymer molecule(s).

“Humanized” forms of non-human (e.g., murine) antibodies are chimeric antibodies that contain minimal sequence derived from non-human immunoglobulin. Or, looked at another way, a humanized antibody is a human antibody that also contains selected sequences from non-human (e.g., murine) antibodies in place of the human sequences. A humanized antibody can include conservative amino acid substitutions or non-natural residues from the same or different species that do not significantly alter its binding and/or biologic activity. Such antibodies are chimeric antibodies that contain minimal sequence derived from non-human immunoglobulins. For the most part, humanized antibodies are human immunoglobulins (recipient antibody) in which residues from a complementary-determining region (CDR) of the recipient are replaced by residues from a CDR of a non-human species (donor antibody) such as mouse, rat, camel, bovine, goat, or rabbit having the desired properties. In some instances, framework region (FR) residues of the human immunoglobulin are replaced by corresponding non-human residues.

Furthermore, humanized antibodies can comprise residues that are found neither in the recipient antibody nor in the imported CDR or framework sequences. These modifications are made to further refine and maximize antibody performance. Thus, in general, a humanized antibody will comprise all of at least one, and in one aspect two, variable domains, in which all or all of the hypervariable loops correspond to those of a non-human immunoglobulin and all or substantially all of the FR regions are those of a human immunoglobulin sequence. The humanized antibody optionally also will comprise at least a portion of an immunoglobulin constant region (Fc), or that of a human immunoglobulin. See, e.g., Cabilly, et al., U.S. Pat. No. 4,816,567; Cabilly, et al., European Patent No. 0,125,023 B1; Boss, et al., U.S. Pat. No. 4,816,397; Boss, et al., European Patent No. 0,120,694 B1; Neuberger, et al., WO 86/01533; Neuberger, et al., European Patent No. 0,194,276 B1; Winter, U.S. Pat. No. 5,225,539; Winter, European Patent No. 0,239,400 B1; Padlan, et al., European Patent Application No. 0,519,596 A1; Queen, et al. (1989), Proc. Nat'l Acad. Sci. USA, vol. 86:10029-10033). For further details, see Jones et al., Nature 321:522-525 (1986); Reichmann et al., Nature 332:323-329 (1988); and Presta, Curr. Op. Struct. Biol. 2:593-596 (1992) and Hansen, WO2006105062.

The term “hyperproliferative disorder” refers to diseases and disorders associated with, the uncontrolled proliferation of cells, including but not limited to uncontrolled growth of organ and tissue cells resulting in cancers and benign tumors. Hyperproliferative disorders associated with endothelial cells can result in diseases of angiogenesis such as angiomas, endometriosis, obesity, age-related macular degeneration and various retinopathies, as well as the proliferation of endothelial cells and smooth muscle cells that cause restenosis as a consequence of stenting in the treatment of atherosclerosis. Hyperproliferative disorders involving fibroblasts (i.e., fibrogenesis) include but are not limited to disorders of excessive scarring (i.e., fibrosis) such as age-related macular degeneration, cardiac remodeling and failure associated with myocardial infarction, excessive wound healing such as commonly occurs as a consequence of surgery or injury, keloids, and fibroid tumors and stenting.

An “immune-derived moiety” includes any antibody (Ab) or immunoglobulin (Ig), and refers to any form of a peptide, polypeptide derived from, modeled after or encoded by, an immunoglobulin gene, or a fragment of such peptide or polypeptide that is capable of binding an antigen or epitope (see, e.g., Immunobiology, 5th Edition, Janeway, Travers, Walport, Shlomchiked. (editors), Garland Publishing (2001)). In the present invention, the antigen is a lipid molecule, such as a bioactive lipid molecule.

An “immunogen” is a molecule capable of inducing a specific immune response, particularly an antibody response in an animal to whom the immunogen has been administered. In the instant invention, the immunogen is a derivatized bioactive lipid conjugated to a carrier, i.e., a “derivatized bioactive lipid conjugate”. The derivatized bioactive lipid conjugate used as the immunogen may be used as capture material for detection of the antibody generated in response to the immunogen. Thus the immunogen may also be used as a detection reagent. Alternatively, the derivatized bioactive lipid conjugate used as capture material may have a different linker and/or carrier moiety from that in the immunogen.

The phrase “in silico” refers to computer simulations that model natural or laboratory processes

To “inhibit,” particularly in the context of a biological phenomenon, means to decrease, suppress or delay. For example, a treatment yielding “inhibition of tumorigenesis” may mean that tumors do not form at all, or that they form more slowly, or are fewer in number than in the untreated control.

An “isolated” antibody is one that has been identified and separated and/or recovered from a component of its natural environment. Contaminant components of its natural environment are materials that would interfere with diagnostic or therapeutic uses for the antibody, and may include enzymes, hormones, and other proteinaceous or nonproteinaceous solutes. In preferred embodiments, the antibody will be purified (1) to greater than 95% by weight of antibody as determined by the Lowry method, and most preferably more than 99% by weight, (2) to a degree sufficient to obtain at least 15 residues of N-terminal or internal amino acid sequence by use of a spinning cup sequenator, or (3) to homogeneity by SDS-PAGE under reducing or nonreducing conditions using Coomassie blue or, preferably, silver stain. Isolated antibody includes the antibody in situ within recombinant cells since at least one component of the antibody's natural environment will not be present. Ordinarily, however, isolated antibody will be prepared by at least one purification step.

The word “label” when used herein refers to a detectable compound or composition, such as one that is conjugated directly or indirectly to the antibody. The label may itself be detectable by itself (e.g., radioisotope labels or fluorescent labels) or, in the case of an enzymatic label, may catalyze chemical alteration of a substrate compound or composition that is detectable.

A “ligand” is a substance that is able to bind to and form a complex with a biomolecule to serve a biological purpose. Thus an antigen may be described as a ligand of the antibody to which it binds.

A “liposome” is a small vesicle composed of various types of lipids, phospholipids and/or surfactant that is useful for delivery of a drug (such as the antibodies disclosed herein and, optionally, a chemotherapeutic agent) to a mammal. The components of the liposome are commonly arranged in a bilayer formation, similar to the lipid arrangement of biological membranes. An “isolated” nucleic acid molecule is a nucleic acid molecule that is identified and separated from at least one contaminant nucleic acid molecule with which it is ordinarily associated in the natural source of the antibody nucleic acid. An isolated nucleic acid molecule is other than in the form or setting in which it is found in nature. Isolated nucleic acid molecules therefore are distinguished from the nucleic acid molecule as it exists in natural cells. However, an isolated nucleic acid molecule includes a nucleic acid molecule contained in cells that ordinarily express the antibody where, for example, the nucleic acid molecule is in a chromosomal location different from that of natural cells.

In the context of this invention, a “liquid composition” refers to one that, in its filled and finished form as provided from a manufacturer to an end user (e.g., a doctor or nurse), is a liquid or solution, as opposed to a solid. Here, “solid” refers to compositions that are not liquids or solutions. For example, solids include dried compositions prepared by lyophilization, freeze-drying, precipitation, and similar procedures.

The expression “linear antibodies” when used throughout this application refers to the antibodies described in Zapata et al. Protein Eng. 8(10):1057-1062 (1995). Briefly, these antibodies comprise a pair of tandem Fd segments (V_{H}-C_{H}1-V_{H}-C_{H}1) that form a pair of antigen binding regions. Linear antibodies can be bispecific or monospecific.

The term “metabolites” refers to compounds from which LPAs are made, as well as those that result from the degradation of LPAs; that is, compounds that are involved in the lysophospholipid metabolic pathways. The term “metabolic precursors” may be used to refer to compounds from which sphingolipids are made.

The term “monoclonal antibody” (mAb) as used herein refers to an antibody obtained from a population of substantially homogeneous antibodies, or to said population of antibodies. The individual antibodies comprising the population are essentially identical, except for possible naturally occurring mutations that may be present in minor amounts. Monoclonal antibodies are highly specific, being directed against a single antigenic site. Furthermore, in contrast to conventional (polyclonal) antibody preparations that typically include different antibodies directed against different determinants (epitopes), each monoclonal antibody is directed against a single determinant on the antigen. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by the hybridoma method first described by Kohler et al., Nature 256:495 (1975), or may be made by recombinant DNA methods (see, e.g., U.S. Pat. No. 4,816,567). The “monoclonal antibodies” may also be isolated from phage antibody libraries using the techniques described in Clackson et al., Nature 352:624-628 (1991) and Marks et al., J. Mol. Biol. 222:581-597 (1991), for example, or by other methods known in the art. The monoclonal antibodies herein specifically include chimeric antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (U.S. Pat. No. 4,816,567; and Morrison et al., Proc. Natl. Acad. Sci. USA 81:6851-6855 (1984)).

“Monotherapy” refers to a treatment regimen based on the delivery of one therapeutically effective compound, whether administered as a single dose or several doses over time.

The term “multispecific antibody” can refer to an antibody, or a monoclonal antibody, having binding properties for at least two different epitopes. In one embodiment, the epitopes are from the same antigen. In another embodiment, the epitopes are from two or more different antigens. Methods for making multispecific antibodies are known in the art. Multispecific antibodies include bispecific antibodies (having binding properties for two epitopes), trispecific antibodies (three epitopes) and so on. For example, multispecific antibodies can be produced recombinantly using the co-expression of two or more immunoglobulin heavy chain/light chain pairs. Alternatively, multispecific antibodies can be prepared using chemical linkage. One of skill can produce multispecific antibodies using these or other methods as may be known in the art. Multispecific antibodies include multispecific antibody fragments. One example of a multispecific (in this case, bispecific) antibody comprehended by this invention is an antibody having binding properties for an S1P epitope and a C1P epitope, which thus is able to recognize and bind to both S1P and C1P. Another example of a bispecific antibody comprehended by this invention is an antibody having binding properties for an epitope from a bioactive lipid and an epitope from a cell surface antigen. Thus the antibody is able to recognize and bind the bioactive lipid and is able to recognize and bind to cells, e.g., for targeting purposes.

“Neoplasia” or “cancer” refers to abnormal and uncontrolled cell growth. A “neoplasm”, or tumor or cancer, is an abnormal, unregulated, and disorganized proliferation of cell growth, and is generally referred to as cancer. A neoplasm may be benign or malignant. A neoplasm is malignant, or cancerous, if it has properties of destructive growth, invasiveness, and metastasis. Invasiveness refers to the local spread of a neoplasm by infiltration or destruction of surrounding tissue, typically breaking through the basal laminas that define the boundaries of the tissues, thereby often entering the body's circulatory system. Metastasis typically refers to the dissemination of tumor cells by lymphatics or blood vessels. Metastasis also refers to the migration of tumor cells by direct extension through serous cavities, or subarachnoid or other spaces. Through the process of metastasis, tumor cell migration to other areas of the body establishes neoplasms in areas away from the site of initial appearance.

Nucleic acid is “operably linked” when it is placed into a functional relationship with another nucleic acid sequence. For example, DNA for a presequence or secretory leader is operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, “operably linked” means that the DNA sequences being linked are contiguous, and, in the case of a secretory leader, contiguous and in reading phase. However, enhancers do not have to be contiguous. Linking is accomplished by ligation at convenient restriction sites. If such sites do not exist, the synthetic oligonucleotide adaptors or linkers are used in accordance with conventional practice.

The “parent” antibody herein is one that is encoded by an amino acid sequence used for the preparation of the variant. The parent antibody may be a native antibody or may already be a variant, e.g., a chimeric antibody. For example, the parent antibody may be a humanized or human antibody.

A “patentable” composition, process, machine, or article of manufacture according to the invention means that the subject matter satisfies all statutory requirements for patentability at the time the analysis is performed. For example, with regard to novelty, non-obviousness, or the like, if later investigation reveals that one or more claims encompass one or more embodiments that would negate novelty, non-obviousness, etc., the claim(s), being limited by definition to “patentable” embodiments, specifically exclude the non-patentable embodiment(s). Also, the claims appended hereto are to be interpreted both to provide the broadest reasonable scope, as well as to preserve their validity. Furthermore, the claims are to be interpreted in a way that (1) preserves their validity and (2) provides the broadest reasonable interpretation under the circumstances, if one or more of the statutory requirements for patentability are amended or if the standards change for assessing whether a particular statutory requirement for patentability is satisfied from the time this application is filed or issues as a patent to a time the validity of one or more of the appended claims is questioned.

The term “pharmaceutically acceptable salt” refers to a salt, such as used in formulation, which retains the biological effectiveness and properties of the agents and compounds of this invention and which are is biologically or otherwise undesirable. In many cases, the agents and compounds of this invention are capable of forming acid and/or base salts by virtue of the presence of charged groups, for example, charged amino and/or carboxyl groups or groups similar thereto. Pharmaceutically acceptable acid addition salts may be prepared from inorganic and organic acids, while pharmaceutically acceptable base addition salts can be prepared from inorganic and organic bases. For a review of pharmaceutically acceptable salts (see Berge, et al., (1977) J. Pharm. Sci., vol. 66, 1-19).

A “plurality” means more than one.

The term “promoter” includes all sequences capable of driving transcription of a coding sequence in a cell. Thus, promoters used in the constructs of the invention include cis-acting transcriptional control elements and regulatory sequences that are involved in regulating or modulating the timing and/or rate of transcription of a gene. For example, a promoter can be a cis-acting transcriptional control element, including an enhancer, a promoter, a transcription terminator, an origin of replication, a chromosomal integration sequence, 5′ and 3′ untranslated regions, or an intronic sequence, which are involved in transcriptional regulation. Transcriptional regulatory regions suitable for use in the present invention include but are not limited to the human cytomegalovirus (CMV) immediate-early enhancer/promoter, the SV40 early enhancer/promoter, the E. coli lac or trp promoters, and other promoters known to control expression of genes in prokaryotic or eukaryotic cells or their viruses.

The term “recombinant DNA” refers to nucleic acids and gene products expressed therefrom that have been engineered, created, or modified by man. “Recombinant” polypeptides or proteins are polypeptides or proteins produced by recombinant DNA techniques, for example, from cells transformed by an exogenous DNA construct encoding the desired polypeptide or protein. “Synthetic” polypeptides or proteins are those prepared by chemical synthesis.

The terms “separated”, “purified”, “isolated”, and the like mean that one or more components of a sample contained in a sample-holding vessel are or have been physically removed from, or diluted in the presence of, one or more other sample components present in the vessel. Sample components that may be removed or diluted during a separating or purifying step include, chemical reaction products, non-reacted chemicals, proteins, carbohydrates, lipids, and unbound molecules.

By “solid phase” is meant a non-aqueous matrix such as one to which the antibody of the present invention can adhere. Examples of solid phases encompassed herein include those formed partially or entirely of glass (e.g. controlled pore glass), polysaccharides (e.g., agarose), polyacrylamides, polystyrene, polyvinyl alcohol and silicones. In certain embodiments, depending on the context, the solid phase can comprise the well of an assay plate; in others it is a purification column (e.g. an affinity chromatography column). This term also includes a discontinuous solid phase of discrete particles, such as those described in U.S. Pat. No. 4,275,149.

The term “species” is used herein in various contexts, e.g., a particular species of chemotherapeutic agent. In each context, the term refers to a population of chemically indistinct molecules of the sort referred in the particular context.

The term “specific” or “specificity” in the context of antibody-antigen interactions refers to the selective, non-random interaction between an antibody and its target epitope. Here, the term “antigen” refers to a molecule that is recognized and bound by an antibody molecule or other immune-derived moiety. The specific portion of an antigen that is bound by an antibody is termed the “epitope”. This interaction depends on the presence of structural, hydrophobic/hydrophilic, and/or electrostatic features that allow appropriate chemical or molecular interactions between the molecules. Thus an antibody is commonly said to “bind” (or “specifically bind”) or be “reactive with” (or “specifically reactive with), or, equivalently, “reactive against” (or “specifically reactive against”) the epitope of its target antigen. Antibodies are commonly described in the art as being “against” or “to” their antigens as shorthand for antibody binding to the antigen. Thus an “antibody that binds C1P,” an “antibody reactive against C1P,” an “antibody reactive with C1P,” an “antibody to C1P” and an “anti-C1P antibody” all have the same meaning in the art. Antibody molecules can be tested for specificity of binding by comparing binding to the desired antigen to binding to unrelated antigen or analogue antigen or antigen mixture under a given set of conditions. Preferably, an antibody according to the invention will lack significant binding to unrelated antigens, or even analogs of the target antigen.

“Specifically associate” and “specific association” and the like refer to a specific, non-random interaction between two molecules, which interaction depends on the presence of structural, hydrophobic/hydrophilic, and/or electrostatic features that allow appropriate chemical or molecular interactions between the molecules.

The term “sphingolipid” as used herein refers to the class of compounds in the art known as sphingolipids, including, but not limited to the following compounds (see http//www.lipidmaps.org for chemical formulas, structural information, etc. for the corresponding compounds):

Sphingoid bases [SP01]

Sphing-4-enines (Sphingosines) [SP0101]

Sphinganines [SP0102]

4-Hydroxysphinganines (Phytosphingosines) [SP0103]

Sphingoid base homologs and variants [SP0104]

Sphingoid base 1-phosphates [SP0105]

Lysosphingomyelins and lysoglycosphingolipids [SP0106]

N-methylated sphingoid bases [SP0107]

Sphingoid base analogs [SP0108]

Ceramides [SP02]

N-acylsphingosines (ceramides) [SP0201]

N-acylsphinganines (dihydroceramides) [SP0202]

N-acyl-4-hydroxysphinganines (phytoceramides) [SP0203]

Acylceramides [SP0204]

Ceramide 1-phosphates [SP0205]

Phosphosphingolipids [SP03]

Ceramide phosphocholines (sphingomyelins) [SP0301]

Ceramide phosphoethanolamines [SP0302]

Ceramide phosphoinositols [SP0303]

Phosphonosphingolipids [SP04]

Neutral glycosphingolipids [SP05]

Simple Glc series (GlcCer, LacCer, etc) [SP0501]

GalNAcb1-3Gala1-4Galb1-4Glc- (Globo series) [SP0502]

GalNAcb1-4Galb1-4Glc- (Ganglio series) [SP0503]

Galb1-3GlcNAcb1-3Galb1-4Glc- (Lacto series) [SP0504]

Galb1-4GlcNAcb1-3Galb1-4Glc- (Neolacto series) [SP0505]

GalNAcb1-3Gala1-3Galb1-4Glc- (Isoglobo series) [SP0506]

GlcNAcb1-2Mana1-3Manb1-4Glc- (Mollu series) [SP0507]

GalNAcb1-4GlcNAcb1-3Manb1-4Glc- (Arthro series) [SP0508]

Gal- (Gala series) [SP0509]

Other [SP0510]

Acidic glycosphingolipids [SP06]

Gangliosides [SP0601]

Sulfoglycosphingolipids (sulfatides) [SP0602]

Glucuronosphingolipids [SP0603]

Phosphoglycosphingolipids [SP0604]

Other [SP0600]

Basic glycosphingolipids [SP07]

Amphoteric glycosphingolipids [SP08]

Arsenosphingolipids [SP09]

The present invention relates to anti-lipid agents, including anti-LPA antibodies, that are useful for treating or preventing hyperproliferative disorders such as cancer and various fibrosis-related disorders, as described in greater detail below. The term “lysolipid metabolite” refers to a compound from which a lysolipid is made, as well as a that results from the degradation of a particular lysolipid. In other words, a “lysolipid metabolite” is a compound that is involved in the lysolipid metabolic pathways. Metabolites include metabolic precursors and metabolic products. The term “metabolic precursors” refers to compounds from which lysolipids are made. The term “metabolic products” refers to compounds that result from the degradation of lysolipids.

Herein, “stable” refers to an interaction between two molecules (e.g., a peptide and a TLR molecule) that is sufficiently stable such that the molecules can be maintained for the desired purpose or manipulation. For example, a “stable” interaction between a peptide and a TLR molecule refers to one wherein the peptide becomes and remains associated with a TLR molecule for a period sufficient to achieve the desired effect.

A “subject” or “patient” refers to an animal in need of treatment that can be effected by molecules of the invention. Animals that can be treated in accordance with the invention include vertebrates, with mammals such as bovine, canine, equine, feline, ovine, porcine, and primate (including humans and non-human primates) animals being particularly preferred examples.

A “surrogate marker” refers to laboratory measurement of biological activity within the body that indirectly indicates the effect of treatment on disease state. Examples of surrogate markers for hyperproliferative and/or cardiovascular conditions include SPHK and/or S1PRs.

A “therapeutic agent” refers to a drug or compound that is intended to provide a therapeutic effect including, but not limited to: anti-inflammatory drugs including COX inhibitors and other NSAIDS, anti-angiogenic drugs, chemotherapeutic drugs as defined above, cardiovascular agents, immunomodulatory agents, agents that are used to treat neurodegenerative disorders, opthalmic drugs, anti-fibrotics, etc.

A “therapeutically effective amount” (or “effective amount”) refers to an amount of an active ingredient, e.g., an agent according to the invention, sufficient to effect treatment when administered to a subject in need of such treatment. Accordingly, what constitutes a therapeutically effective amount of a composition according to the invention may be readily determined by one of ordinary skill in the art. In the context of cancer therapy, a “therapeutically effective amount” is one that produces an objectively measured change in one or more parameters associated with cancer cell survival or metabolism, including an increase or decrease in the expression of one or more genes correlated with the particular cancer, reduction in tumor burden, cancer cell lysis, the detection of one or more cancer cell death markers in a biological sample (e.g., a biopsy and an aliquot of a bodily fluid such as whole blood, plasma, serum, urine, etc.), induction of induction apoptosis or other cell death pathways, etc. Of course, the therapeutically effective amount will vary depending upon the particular subject and condition being treated, the weight and age of the subject, the severity of the disease condition, the particular compound chosen, the dosing regimen to be followed, timing of administration, the manner of administration and the like, all of which can readily be determined by one of ordinary skill in the art. It will be appreciated that in the context of combination therapy, what constitutes a therapeutically effective amount of a particular active ingredient may differ from what constitutes a therapeutically effective amount of the active ingredient when administered as a monotherapy (i.e., a therapeutic regimen that employs only one chemical entity as the active ingredient).

The compositions of the invention are used in methods of bioactive lipid-based therapy. As used herein, the terms “therapy” and “therapeutic” encompasses the full spectrum of prevention and/or treatments for a disease, disorder or physical trauma. A “therapeutic” agent of the invention may act in a manner that is prophylactic or preventive, including those that incorporate procedures designed to target individuals that can be identified as being at risk (pharmacogenetics); or in a manner that is ameliorative or curative in nature; or may act to slow the rate or extent of the progression of at least one symptom of a disease or disorder being treated; or may act to minimize the time required, the occurrence or extent of any discomfort or pain, or physical limitations associated with recuperation from a disease, disorder or physical trauma; or may be used as an adjuvant to other therapies and treatments. The term “treatment” or “treating” means any treatment of a disease or disorder, including preventing or protecting against the disease or disorder (that is, causing the clinical symptoms not to develop); inhibiting the disease or disorder (i.e., arresting, delaying or suppressing the development of clinical symptoms; and/or relieving the disease or disorder (i.e., causing the regression of clinical symptoms). As will be appreciated, it is not always possible to distinguish between “preventing” and “suppressing” a disease or disorder because the ultimate inductive event or events may be unknown or latent. Those “in need of treatment” include those already with the disorder as well as those in which the disorder is to be prevented. Accordingly, the term “prophylaxis” will be understood to constitute a type of “treatment” that encompasses both “preventing” and “suppressing”. The term “protection” thus includes “prophylaxis”.

The term “therapeutic regimen” means any treatment of a disease or disorder using chemotherapeutic and cytotoxic agents, radiation therapy, surgery, gene therapy, DNA vaccines and therapy, siRNA therapy, anti-angiogenic therapy, immunotherapy, bone marrow transplants, aptamers and other biologics such as antibodies and antibody variants, receptor decoys and other protein-based therapeutics.

The “variable” region of an antibody comprises framework and complementarity determining regions (CDRs, otherwise known as hypervariable regions). The variability is not evenly distributed throughout the variable domains of antibodies. It is concentrated in six CDR segments, three in each of the light chain and the heavy chain variable domains. The more highly conserved portions of variable domains are called the framework region (FR). The variable domains of native heavy and light chains each comprise four FRs (FR1, FR2, FR3 and FR4, respectively), largely adopting a 13-sheet configuration, connected by three hypervariable regions, which form loops connecting, and in some cases forming part of, the beta-sheet structure. The term “hypervariable region” when used herein refers to the amino acid residues of an antibody which are responsible for antigen binding. The hypervariable region comprises amino acid residues from a “complementarity determining region” or “CDR” (for example residues 24-34 (L1), 50-56 (L2) and 89-97 (L3) in the light chain variable domain and 31-35 (H1), 50-65 (H2) and 95-102 (H3) in the heavy chain variable domain; Kabat et al., Sequences of Proteins of Immunological Interest, 5th Ed. Public Health Service, National Institutes of Health, Bethesda, Md. (1991)) and/or those residues from a “hypervariable loop” (for example residues 26-32 (L1), 50-52 (L2) and 91-96 (L3) in the light chain variable domain and 26-32 (H1), 53-55 (H2) and 96-101 (H3) in the heavy chain variable domain; Chothia and Lesk J. Mol. Biol. 196:901-917 (1987)). “Framework” or “FR” residues are those variable domain residues other than the hypervariable region residues as herein defined.

It should be noted that, in the art, more than one system for numbering of amino acid residues is commonly used. The CDRs above are described and numbered according to the Kabat numbering scheme (Kabat et al., Sequences of Proteins of Immunological Interest, 5th Ed. Public Health Service, National Institutes of Health, Bethesda, Md. (1991)) but sequential numbering may also be used. Sequential and Kabat numbering are identical for the entire LT1009 light chain, and up to position 52 in the LT1009 heavy chain. In the heavy chain (VH), according to Kabat numbering there is a single residue insertion after position 52, a three-residue insertion after position 82 and a four residue insertion after position 100. Thus residues may at times be seen to be numbered 52A, 100A, 100C etc. to reflect these insertions according to the Kabat system.

The hypervariable regions in each chain are held together in close proximity by the FRs and, with the hypervariable regions from the other chain, contribute to the formation of the antigen-binding site of antibodies (see Kabat et al., Sequences of Proteins of Immunological Interest, 5th Ed. Public Health Service, National Institutes of Health, Bethesda, Md. (1991), pages 647-669). The constant domains are not involved directly in binding an antibody to an antigen, but exhibit various effector functions, such as participation of the antibody in antibody-dependent cellular toxicity.

A “vector” or “plasmid” or “expression vector” refers to a nucleic acid that can be maintained transiently or stably in a cell to effect expression of one or more recombinant genes. A vector can comprise nucleic acid, alone or complexed with other compounds. A vector optionally comprises viral or bacterial nucleic acids and/or proteins, and/or membranes. Vectors include, but are not limited, to replicons (e.g., RNA replicons, bacteriophages) to which fragments of DNA may be attached and become replicated. Thus, vectors include, but are not limited to, RNA, autonomous self-replicating circular or linear DNA or RNA and include both the expression and non-expression plasmids. Plasmids can be commercially available, publicly available on an unrestricted basis, or can be constructed from available plasmids as reported with published protocols. In addition, the expression vectors may also contain a gene to provide a phenotypic trait for selection of transformed host cells such as dihydrofolate reductase or neomycin resistance for eukaryotic cell culture, or such as tetracycline or ampicillin resistance in E. coli.

### SUMMARY OF THE INVENTION

The present invention provides patentable crystalline forms of an anti-LPA antibody or fragment thereof, which may further comprise a lipid ligand of said antibody. Methods for making such crystals are provided, as is structural information obtained from such crystals. Methods of using this information in antibody design or optimization are provided.

Methods for designing a humanized antibody to a lipid are provided, which may be performed in silico. These methods may result in enhanced binding affinity of an antibody to its original target lipid, or may be intended to alter binding specificity. Optimized humanized monoclonal antibodies to LPA are also provided.

These and other aspects and embodiments of the invention are discussed in greater detail in the sections that follow. The foregoing and other aspects of the invention will become more apparent from the following detailed description, accompanying drawings, and the claims. Although methods and materials similar or equivalent to those described herein can be used in the practice or testing of the present invention, suitable methods and materials are described below. In addition, the materials, methods, and examples below are illustrative only and not intended to be limiting.

### BRIEF DESCRIPTION OF THE DRAWINGS

This application contains at least one figure executed in color. Copies of this application with color drawing(s) will be provided upon request and payment of the necessary fee. A brief summary of each of the figures is provided below.

FIG. 1: Crystal structures of the (A) 1.9 Å S1P-FAb complex (for comparison), (B) 2.0 Å14:0 LPA-Fab complex, (C) 2.5 Å 18:2 LPA-Fab complex and (D) 2.3 Å LT3015 Fab in the absence of lipid. The Fab backbone atoms are traced with a ribbon and the lipids are shown as a ball and stick (A) or space filling diagram (B and C). All structures are shown from the same view and the heavy and light chains are labeled in (A).

FIG. 2: Structures of the lipid-binding sites. The electron density (blue) from 2|FO|−|FC| refined maps contoured at (A) 1.1 s for the S1P-Fab complex, (B) 1.2 s for the 14:0 LPA-Fab complex and (C) 1.0 s for the 18:2 LPA-Fab complex.

### DETAILED DESCRIPTION OF THE INVENTION

1. Antibody Compounds.

Antibody molecules or immunoglobulins are large glycoprotein molecules with a molecular weight of approximately 150 kDa, usually composed of two different kinds of polypeptide chain. The heavy chain (H) is approximately 50 kDa. The light chain (L), is approximately 25 kDa. Each immunoglobulin molecule usually consists of two heavy chains and two light chains. The two heavy chains are linked to each other by disulfide bonds, the number of which varies between the heavy chains of different immunoglobulin isotypes. Each light chain is linked to a heavy chain by one covalent disulfide bond. In any given naturally occurring antibody molecule, the two heavy chains and the two light chains are identical, harboring two identical antigen-binding sites, and are thus said to be divalent, i.e., having the capacity to bind simultaneously to two identical molecules.

The light chains of antibody molecules from any vertebrate species can be assigned to one of two clearly distinct types, kappa (k) and lambda (l), based on the amino acid sequences of their constant domains. The ratio of the two types of light chain varies from species to species. As a way of example, the average k to l ratio is 20:1 in mice, whereas in humans it is 2:1 and in cattle it is 1:20.

The heavy chains of antibody molecules from any vertebrate species can be assigned to one of five clearly distinct types, called isotypes, based on the amino acid sequences of their constant domains. Some isotypes have several subtypes. The five major classes of immunoglobulin are immunoglobulin M (IgM), immunoglobulin D (IgD), immunoglobulin G (IgG), immunoglobulin A (IgA), and immunoglobulin E (IgE). IgG is the most abundant isotype and has several subclasses (IgG1, 2, 3, and 4 in humans). The Fc fragment and hinge regions differ in antibodies of different isotypes, thus determining their functional properties. However, the overall organization of the domains is similar in all isotypes.

Sources of antibody are not limited to those exemplified herein (e.g., murine and humanized murine antibody). Antibodies may be raised in many species including mammalian species (for example, mouse, rat, camel, bovine, goat, horse, guinea pig, hamster, sheep and rabbit) and birds (duck, chicken). Antibodies raised may derive from a different species from the animal in which they are raised. For example, the XenoMouse™ (Abgenix, Inc., Fremont Calif.) produces fully human monoclonal antibodies. For certain purposes, native human antibodies, such as autoantibodies to S1β isolated from individuals who may show a titer of such S1P autoantibody may be used. Alternatively, a human antibody sequence library may be used to generate antibodies comprising a human sequence.

2. Antibody Applications.

Therapeutic agents that alter the activity or concentration of one or more undesired bioactive lipids, or precursors or metabolites thereof, are therapeutically useful. These agents, including antibodies, act by changing the effective concentration, i.e., the absolute, relative, effective and/or available concentration and/or activities, of certain undesired bioactive lipids. Lowering the effective concentration of the bioactive lipid may be said to “neutralize” the target lipid or its undesired effects, including downstream effects. Here, “undesired” refers to a bioactive lipid that is unwanted due to its involvement in a disease process, for example, as a signaling molecule, or to an unwanted amount of a bioactive lipid which contributes to disease when present in excess.

Without wishing to be bound by any particular theory, it is believed that inappropriate concentrations of bioactive lipids, such as LPA and/or its metabolites or downstream effectors, may cause or contribute to the development of various diseases and disorders. As such, the compositions and methods can be used to treat these diseases and disorders, particularly by decreasing the effective in vivo concentration of a particular target lipid, for example, LPA or its variants. In particular, agents that reduce the effective concentration of LPA, such as Lpath's anti-LPA mAbs, are believed to be useful in methods for treating diseases and conditions such as those associated with wound healing and fibrosis, apoptosis, angiogenesis and neovascularizaion, vascular permeability and inflammation, that are associated with an undesired, excessive or aberrant level of LPA.

Other examples of diseases that may be treated with antibodies targeted to bioactive lipid are described below in applicant's pending patent applications and issued patents. See, for example, U.S. patent application Ser. Nos. 11/755,721, filed 30 May 2007, (PG Pub 20080145360, attorney docket no. LPT-3100-UT4), 12/129,109, filed 29 May 2008 (PGPub 20090136483, attorney docket no. LPT-3200-UT) and 12/406,874, filed 18 Mar. 2009, (US20100034814, Attorney Docket No. LPT-3200-CP) which are hereby incorporated by reference in their entirety and for all purposes.

One way to control the amount of undesirable lysolipids or other bioactive lipids in a patient is by providing a composition that comprises one or more humanized anti-lysolipid antibodies to bind one or more lysolipids, thereby acting as therapeutic “sponges” that reduce the level of free undesirable lysolipids. When a compound is referred to as “free”, the compound is not in any way restricted from reaching the site or sites where it exerts its undesirable effects. Typically, a free compound is present in blood and tissue, which either is or contains the site(s) of action of the free compound, or from which a compound can freely migrate to its site(s) of action. A free compound may also be available to be acted upon by any enzyme that converts the compound into an undesirable compound.

One form of LPA-based therapy involves manipulating the metabolic pathways of LPA in order to decrease the actual, relative and/or available in vivo concentrations of undesirable LPA. The invention provides compositions and methods for treating or preventing diseases, disorders or physical trauma, in which humanized anti-LPA antibodies are administered to a patient to bind undesirable LPA, or metabolites thereof.

Such humanized anti-LPA antibodies may be formulated in a pharmaceutical composition and are useful for a variety of purposes, including the treatment of diseases, disorders or physical trauma. Pharmaceutical compositions comprising one or more humanized anti-LPA antibodies of the invention may be incorporated into kits and medical devices for such treatment. Medical devices may be used to administer the pharmaceutical compositions of the invention to a patient in need thereof, and according to one embodiment of the invention, kits are provided that include such devices. Such devices and kits may be designed for routine administration, including self-administration, of the pharmaceutical compositions of the invention. Such devices and kits may also be designed for emergency use, for example, in ambulances or emergency rooms, or during surgery, or in activities where injury is possible but where full medical attention may not be immediately forthcoming (for example, hiking and camping, or combat situations).

3. Methods of Administration.

The treatment for diseases and conditions discussed herein can be achieved by administering agents and compositions of the invention by various routes employing different formulations and devices. Suitable pharmaceutically acceptable diluents, carriers, and excipients are well known in the art.

One skilled in the art will appreciate that the amounts to be administered for any particular treatment protocol can readily be determined. Suitable amounts might be expected to fall within the range of 10 μg/dose to 10 g/dose, preferably within 10 mg/dose to 1 g/dose.

Drug substances may be administered by techniques known in the art, including but not limited to systemic, subcutaneous, intradermal, mucosal, including by inhalation, and topical administration. The mucosa refers to the epithelial tissue that lines the internal cavities of the body. For example, the mucosa comprises the alimentary canal, including the mouth, esophagus, stomach, intestines, and anus; the respiratory tract, including the nasal passages, trachea, bronchi, and lungs; and the genitalia. For the purpose of this specification, the mucosa also includes the external surface of the eye, i.e., the cornea and conjunctiva. Local administration (as opposed to systemic administration) may be advantageous because this approach can limit potential systemic side effects, but still allow therapeutic effect.

Pharmaceutical compositions used in the present invention include, but are not limited to, solutions, emulsions, and liposome-containing formulations. These compositions may be generated from a variety of components that include, but are not limited to, preformed liquids, self-emulsifying solids and self-emulsifying semisolids.

The pharmaceutical formulations used in the present invention may be prepared according to conventional techniques well known in the pharmaceutical industry. Such techniques include the step of bringing into association the active ingredients with the pharmaceutical carrier(s) or excipient(s). Preferred carriers include those that are pharmaceutically acceptable, particularly when the composition is intended for therapeutic use in humans. For non-human therapeutic applications (e.g., in the treatment of companion animals, livestock, fish, or poultry), veterinarily acceptable carriers may be employed. In general the formulations are prepared by uniformly and intimately bringing into association the active ingredients with liquid carriers or finely divided solid carriers or both, and then, if necessary, shaping the product.

The compositions of the present invention may be formulated into any of many possible dosage forms such as, but not limited to, tablets, capsules, liquid syrups, soft gels, suppositories, and enemas. The compositions of the present invention may also be formulated as suspensions in aqueous, non-aqueous or mixed media. Aqueous suspensions may further contain substances which increase the viscosity of the suspension including, for example, sodium carboxymethylcellulose, sorbitol and/or dextran. The suspension may also contain stabilizers.

In one embodiment the pharmaceutical compositions may be formulated and used as foams. Pharmaceutical foams include formulations such as, but not limited to, emulsions, microemulsions, creams, jellies, and liposomes.

While basically similar in nature these formulations vary in the components and the consistency of the final product. The know-how on the preparation of such compositions and formulations is generally known to those skilled in the pharmaceutical and formulation arts and may be applied to the formulation of the compositions of the present invention.

In one embodiment, an immune-derived moiety can be delivered to the eye via, for example, topical drops or ointment, periocular injection, intracamerally into the anterior chamber or vitreous, via an implanted depot, or systemically by injection or oral administration. The quantity of antibody used can be readily determined by one skilled in the art.

4. Conventional Antibody Generation and Characterization

Antibody affinities may be determined as described in the examples herein below. Preferred humanized or variant antibodies are those which bind a target bioactive lipid with a K_{d} value of no more than about 1×10⁻⁷ M, preferably no more than about 1×10⁻⁹ M, and most preferably no more than about 5×10⁻⁹ M.

Aside from antibodies with strong binding affinity for a desired bioactive lipid, it is also desirable to select humanized or variant antibodies that have other beneficial properties from a therapeutic perspective. For example, the antibody may be one that reduce angiogenesis and alter tumor progression. Preferably, the antibody has an effective concentration 50 (EC50) value of no more than about 10 ug/ml, preferably no more than about 1 ug/ml, and most preferably no more than about 0.1 ug/ml, as measured in a direct binding ELISA assay. Assays for determining the activity of the antibodies of the invention include ELISA assays as shown in the examples hereinbelow.

Preferably the humanized or variant antibody fails to elicit an immunogenic response upon administration of a therapeutically effective amount of the antibody to a human patient. If an immunogenic response is elicited, preferably the response will be such that the antibody still provides a therapeutic benefit to the patient treated therewith.

According to one embodiment of the invention, humanized antibodies bind the “epitope” as herein defined. To screen for antibodies that bind to the epitope on an LPA bound by an antibody of interest (e.g., those that block binding of the antibody to sphingolipid), a routine cross-blocking assay such as that described in Antibodies, A Laboratory Manual, Cold Spring Harbor Laboratory, Ed Harlow and David Lane (1988), can be performed. Alternatively, epitope mapping, e.g., as described in Champe, et al. [J. Biol. Chem. 270:1388-1394 (1995)], can be performed to determine whether the antibody binds an epitope of interest.

The antibodies of the invention have a heavy chain variable domain comprising an amino acid sequence represented by the formula: FR1-CDRH1-FR2-CDRH2-FR3-CDRH3-FR4, wherein “FR1-4” represents the four framework regions and

“CDRH1-3” represents the three hypervariable regions of an anti-lysolipid antibody variable heavy domain. FR1-4 may be derived from a “consensus sequence” (for example the most common amino acids of a class, subclass or subgroup of heavy or light chains of human immunoglobulins) as in the examples below or may be derived from an individual human antibody framework region or from a combination of different framework region sequences. Many human antibody framework region sequences are compiled in Kabat, et al., supra, for example. In one embodiment, the variable heavy FR is provided by a consensus sequence of a human immunoglobulin subgroup as compiled by Kabat, et al., above.

The human variable heavy FR sequence preferably has one or more substitutions therein, e.g., wherein the human FR residue is replaced by a corresponding nonhuman residue (by “corresponding nonhuman residue” is meant the nonhuman residue with the same Kabat positional numbering as the human residue of interest when the human and nonhuman sequences are aligned), but replacement with the nonhuman residue is not necessary. For example, a replacement FR residue other than the corresponding nonhuman residue can be selected by phage display. Exemplary variable heavy FR residues which may be substituted include any one or more of FR residue numbers: 37H, 49H, 67H, 69H, 71H, 73H, 75H, 76H, 78H, and 94H (Kabat residue numbering employed here). Preferably at least two, or at least three, or at least four of these residues are substituted. A particularly preferred combination of FR substitutions is: 49H, 69H, 71H, 73H, 76H, 78H, and 94H. With respect to the heavy chain hypervariable regions, these preferably have amino acid sequences listed in the examples below.

The antibodies of the preferred embodiment herein have a light chain variable domain comprising an amino acid sequence represented by the formula: FR1-CDRL1-FR2-CDRL2-FR3-CDRL3-FR4, wherein “FR1-4” represents the four framework regions and “CDRL1-3” represents the three hypervariable regions of an anti-lysolipid antibody variable heavy domain. FR1-4 may be derived from a “consensus sequence” (for example, the most common amino acids of a class, subclass or subgroup of heavy or light chains of human immunoglobulins) as in the examples below or may be derived from an individual human antibody framework region or from a combination of different framework region sequences. In one preferred embodiment, the variable light FR is provided by a consensus sequence of a human immunoglobulin subgroup as compiled by Kabat, et al., above.

The human variable light FR sequence preferably has substitutions therein, e.g., wherein a human FR residue is replaced by a corresponding mouse residue, but replacement with the nonhuman residue is not necessary. For example, a replacement residue other than the corresponding nonhuman residue may be selected by phage display. Exemplary variable light FR residues that may be substituted include any one or more of FR residue numbers, including, but not limited to, F4, Y36, Y49, G64, S67.

Methods for generating humanized anti-lysolipid antibodies of interest herein are elaborated in more detail below.

### A. Antibody Preparation

Methods for humanizing nonhuman anti-LPA antibodies and generating variants of anti-LPA antibodies are described in the Examples below. In order to humanize an anti-LPA antibody, the nonhuman antibody starting material is prepared. Where a variant is to be generated, the parent antibody is prepared. Exemplary techniques for generating such nonhuman antibody starting material and parent antibodies will be described in the following sections.

(i) Antigen Preparation.

The bioactive lipid antigen to be used for production of antibodies may be, e.g., intact lipid or a portion of the lipid (e.g., an LPA fragment comprising an “epitope”). Other forms of antigens useful for generating antibodies will be apparent to those skilled in the art. The antigen used to generate the antibody, is described in the examples below. In one embodiment, the antigen is a derivatized form of the bioactive lipid, and may be associated with a carrier protein.

(ii) Polyclonal Antibodies.

Polyclonal antibodies are preferably raised in animals by multiple subcutaneous (sc) or intraperitoneal (ip) injections of the relevant antigen and an adjuvant. It may be useful to conjugate the relevant antigen to a protein that is immunogenic in the species to be immunized, e.g., keyhole limpet hemocyanin, serum albumin, bovine thyroglobulin, or soybean trypsin inhibitor using a bifunctional or derivatizing agent, for example, maleimidobenzoyl sulfosuccinimide ester (conjugation through cysteine residues), N-hydroxysuccinimide (through lysine residues), glutaraldehyde, succinic anhydride, SOCl₂, or R¹N═C=NR, where R and R¹ are different alkyl groups.

Animals are immunized against the antigen, immunogenic conjugates, or derivatives by combining, e.g., 100 ug or 5 ug of the protein or conjugate (for rabbits or mice, respectively) with three volumes of Freund's complete adjuvant and injecting the solution intradermally at multiple sites. One month later the animals are boosted with 0.1 to 0.2 times the original amount of peptide or conjugate in Freund's complete adjuvant by subcutaneous injection at multiple sites. Seven to 14 days later the animals are bled and the serum is assayed for antibody titer. Animals are boosted until the titer plateaus. Preferably, the animal is boosted with the conjugate of the same antigen, but conjugated to a different protein and/or through a different cross-linking reagent. Conjugates also can be made in recombinant cell culture as protein fusions. Also, aggregating agents such as alum may be suitably used to enhance the immune response.

(iii) Monoclonal Antibodies.

Monoclonal antibodies may be made using the hybridoma method first described by Kohler, et al., Nature, 256:495 (1975), or by other suitable methods, including by recombinant DNA methods (see, e.g., U.S. Pat. No. 4,816,567). In the hybridoma method, a mouse or other appropriate host animal, such as a hamster or macaque monkey, is immunized as hereinabove described to elicit lymphocytes that produce or are capable of producing antibodies that will specifically bind to the protein used for immunization. Alternatively, lymphocytes may be immunized in vitro. Lymphocytes then are fused with myeloma cells using a suitable fusing agent, such as polyethylene glycol, to form a hybridoma cell (Goding, Monoclonal Antibodies: Principles and Practice, pp. 59-103 (Academic Press, 1986)).

The hybridoma cells thus prepared are seeded and grown in a suitable culture medium that preferably contains one or more substances that inhibit the growth or survival of the unfused, parental myeloma cells. For example, if the parental myeloma cells lack the enzyme hypoxanthine guanine phosphoribosyl transferase (HGPRT or HPRT), the culture medium for the hybridomas typically will include hypoxanthine, aminopterin, and thymidine (HAT medium), which substances prevent the growth of HGPRT-deficient cells.

Preferred myeloma cells are those that fuse efficiently, support stable high-level production of antibody by the selected antibody-producing cells, and are sensitive to a medium such as HAT medium. Among these, preferred myeloma cell lines are murine myeloma lines, such as those derived from MOP-21 and M.C.-11 mouse tumors available from the Salk Institute Cell Distribution Center, San Diego, Calif. USA, and SP-2 or X63-Ag8-653 cells available from the American Type Culture Collection, Rockville, Md. USA. Human myeloma and mouse-human heteromyeloma cell lines also have been described for the production of human monoclonal antibodies (Kozbor, J. Immunol., 133:3001 (1984); Brodeur, et al., Monoclonal Antibody Production Techniques and Applications, pp. 51-63 (Marcel Dekker, Inc., New York, 1987)).

Culture medium in which hybridoma cells are growing is assayed for production of monoclonal antibodies directed against the antigen. Preferably, the binding specificity of monoclonal antibodies produced by hybridoma cells is determined by immunoprecipitation or by an in vitro binding assay, such as radioimmunoassay (RIA) or enzyme-linked immunoabsorbant assay (ELISA).

The binding affinity of a monoclonal antibody can, for example, be determined by the Scatchard analysis of Munson, et al., Anal. Biochem., 107:220 (1980).

After hybridoma cells are identified that produce antibodies of the desired specificity, affinity, and/or activity, the clones may be subcloned by limiting dilution procedures and grown by standard methods (Goding, Monoclonal Antibodies: Principles and Practice, pp. 59-103 (Academic Press, 1986)). Suitable culture media for this purpose include, for example, D-MEM or RPMI-1640 medium. In addition, the hybridoma cells may be grown in vivo as ascites tumors in an animal.

The monoclonal antibodies secreted by the subclones are suitably separated from the culture medium, ascites fluid, or serum by conventional immunoglobulin purification procedures such as, for example, protein A-Sepharose, hydroxylapatite chromatography, gel electrophoresis, dialysis, or affinity chromatography.

DNA encoding the monoclonal antibodies is readily isolated and sequenced using conventional procedures (e.g., by using oligonucleotide probes that are capable of binding specifically to genes encoding the heavy and light chains of the monoclonal antibodies). The hybridoma cells serve as a preferred source of such DNA. Once isolated, the DNA may be placed into expression vectors, which are then transfected into host cells such as E. coli cells, simian COS cells, Chinese hamster ovary (CHO) cells, or myeloma cells that do not otherwise produce immunoglobulin protein, to obtain the synthesis of monoclonal antibodies in the recombinant host cells. Recombinant production of antibodies will be described in more detail below.

(iv) Humanization and Amino Acid Sequence Variants.

General methods for antibody humanization are described in, for example, U.S. Pat. No. 5,861,155, US19960652558, U.S. Pat. No. 6,479,284, US20000660169, U.S. Pat. No. 6,407,213, US19930146206, U.S. Pat. No. 6,639,055, US20000705686, U.S. Pat. No. 6,500,931, US19950435516, U.S. Pat. No. 5,530,101, U.S. Pat. No. 5,585,089, US19950477728, U.S. Pat. No. 5,693,761, US19950474040, U.S. Pat. No. 5,693,762, US19950487200, U.S. Pat. No. 6,180,370, US19950484537, US2003229208, US20030389155, U.S. Pat. No. 5,714,350, US19950372262, U.S. Pat. No. 6,350,861, US19970862871, U.S. Pat. No. 5,777,085, US19950458516, U.S. Pat. No. 5,834,597, US19960656586, U.S. Pat. No. 5,882,644, US19960621751, U.S. Pat. No. 5,932,448, US19910801798, U.S. Pat. No. 6,013,256, US19970934841, U.S. Pat. No. 6,129,914, US19950397411, U.S. Pat. No. 6,210,671, U.S. Pat. No. 6,329,511, US19990450520, US2003166871, US20020078757, U.S. Pat. No. 5,225,539, US19910782717, U.S. Pat. No. 6,548,640, US19950452462, U.S. Pat. No. 5,624,821, and US19950479752. In certain embodiments, it may be desirable to generate amino acid sequence variants of these humanized antibodies, particularly where these improve the binding affinity or other biological properties of the humanized antibody. Examples hereinbelow describe methodologies for generating amino acid sequence variants of an anti-LPA antibody with enhanced affinity relative to the parent antibody.

Amino acid sequence variants of the anti-LPA antibody are prepared by introducing appropriate nucleotide changes into the anti-LPA antibody DNA, or by peptide synthesis. Such variants include, for example, deletions from, and/or insertions into and/or substitutions of, residues within the amino acid sequences of the anti-LPA antibodies of the examples herein. Any combination of deletion, insertion, and substitution is made to arrive at the final construct, provided that the final construct possesses the desired characteristics. The amino acid changes also may alter post-translational processes of the humanized or variant anti-LPA antibody, such as changing the number or position of glycosylation sites.

A useful method for identification of certain residues or regions of the anti-LPA antibody that are preferred locations for mutagenesis is called “alanine scanning mutagenesis,” as described by Cunningham and Wells Science, 244:1081-1085 (1989). Here, a residue or group of target residues are identified (e.g., charged residues such as arg, asp, his, lys, and glu) and replaced by a neutral or negatively charged amino acid (most preferably alanine or polyalanine) to affect the interaction of the amino acids with lipid antigen. Those amino acid locations demonstrating functional sensitivity to the substitutions then are refined by introducing further or other variants at, or for, the sites of substitution. Thus, while the site for introducing an amino acid sequence variation is predetermined, the nature of the mutation per se need not be predetermined. For example, to analyze the performance of a mutation at a given site, ala scanning or random mutagenesis is conducted at the target codon or region and the expressed anti-lysolipid antibody variants are screened for the desired activity. Amino acid sequence insertions include amino- and/or carboxyl-terminal fusions ranging in length from one residue to polypeptides containing a hundred or more residues, as well as intrasequence insertions of single or multiple amino acid residues. Examples of terminal insertions include an anti-LPA antibody with an N-terminal methionyl residue or the antibody fused to an epitope tag. Other insertional variants of the anti-LPA antibody molecule include the fusion of an enzyme, or a polypeptide which increases the serum half-life of the antibody, to the N- or C-terminus of the antibody.

Another type of variant is an amino acid substitution variant. These variants have at least one amino acid residue in the anti-LPA antibody molecule removed and a different residue inserted in its place. The sites of greatest interest for substitutional mutagenesis include the hypervariable regions, but FR alterations are also contemplated. Conservative substitutions are preferred substitutions. If such substitutions result in a change in biological activity, then more substantial changes, denominated “exemplary” substitutions listed below, or as further described below in reference to amino acid classes, may be introduced and the products screened.

TABLE 1
Exemplary Amino Acid Residue Substitutions

| Amino acid residue (symbol) | Exemplary substitutions |
|---|---|
| Ala (A) | val; leu; ile val |
| Arg (R) | lys; gln; asn lys |
| Asn (N) | gln; his; asp, lys; gln arg |
| Asp (D) | glu; asn glu |
| Cys (C) | ser; ala ser |
| Gln (Q) | asn; glu asn |
| Glu (E) | asp; gln asp |
| Gly (G) | ala ala |
| His (H) | asn; gln; lys; arg arg |
| Ile (I) | leu; val; met; ala; leu phe; norleucine |
| Leu (L) | norleucine; ile; val; ile met; ala; phe |
| Lys (K) | arg; gln; asn arg |
| Met (M) | leu; phe; ile leu |
| Phe (F) | leu; val; ile; ala; tyr tyr |
| Pro (P) | ala ala |
| Ser (S) | thr thr |
| Thr (T) | ser ser |
| Trp (W) | tyr; phe tyr |
| Tyr (Y) | trp; phe; thr; ser phe |
| Val (V) | ile; leu; met; phe; leu ala; norleucine |

Substantial modifications in the biological properties of the antibody are accomplished by selecting substitutions that differ significantly in their effect on maintaining (a) the structure of the polypeptide backbone in the area of the substitution, for example, as a sheet or helical conformation, (b) the charge or hydrophobicity of the molecule at the target site, or (c) the bulk of the side chain. Naturally occurring residues are divided into groups based on common side-chain properties:

(1) hydrophobic: norleucine, met, ala, val, leu, ile;

(2) neutral hydrophilic: cys, ser, thr;

(3) acidic: asp, glu;

(4) basic: asn, gln, his, lys, arg;

(5) residues that influence chain orientation: gly, pro; and

(6) aromatic: trp, tyr, phe.

Non-conservative substitutions will entail exchanging a member of one of these classes for another class.

Any cysteine residue not involved in maintaining the proper conformation of the humanized or variant antibody also may be substituted, to improve the oxidative stability of the molecule and prevent aberrant crosslinking. Conversely, cysteine bond(s) may be added to the antibody to improve its stability (particularly where the antibody is an antibody fragment such as an Fv fragment).

One type of substitutional variant involves substituting one or more hypervariable region residues of a parent antibody (e.g., a humanized or human antibody). Generally, the resulting variant(s) selected for further development will have improved biological properties relative to the parent antibody from which they are generated. A convenient way for generating such substitutional variants is affinity maturation using phage display. Briefly, several hypervariable region sites (e.g., 6-7 sites) are mutated to generate all possible amino substitutions at each site. The antibody variants thus generated are displayed in a monovalent fashion from filamentous phage particles as fusions to the gene IIII product of M13 packaged within each particle. The phage-displayed variants are then screened for their biological activity (e.g., binding affinity) as herein disclosed. In order to identify candidate hypervariable region sites for modification, alanine scanning mutagenesis can be performed to identify hypervariable region residues contributing significantly to antigen binding. Alternatively, or in addition, it may be beneficial to analyze a crystal structure of the antigen-antibody complex to identify contact points between the antibody and lipid. Such contact residues and neighboring residues are candidates for substitution according to the techniques elaborated herein. Crystals (co-crystals) of the antigen—antibody complex include co-crystals of the antigen and the Fab or other fragment of the antibody, along with any salts, metals (including divalent metals), cofactors and the like. Once such variants are generated, the panel of variants is subjected to screening as described herein and antibodies with superior properties in one or more relevant assays may be selected for further development.

Another type of amino acid variant of the antibody alters the original glycosylation pattern of the antibody. By altering is meant deleting one or more carbohydrate moieties found in the antibody, and/or adding one or more glycosylation sites that are not present in the antibody.

Glycosylation of antibodies is typically either N-linked and/or or O-linked. N-linked refers to the attachment of the carbohydrate moiety to the side chain of an asparagine residue. The tripeptide sequences asparagine-X-serine and asparagine-X-threonine, where X is any amino acid except proline, are the most common recognition sequences for enzymatic attachment of the carbohydrate moiety to the asparagine side chain. Thus, the presence of either of these tripeptide sequences in a polypeptide creates a potential glycosylation site. O-linked glycosylation refers to the attachment of one of the sugars N-aceylgalactosamine, galactose, or xylose to a hydroxyamino acid, most commonly serine or threonine, although 5-hydroxyproline or 5-hydroxylysine may also be used.

Addition of glycosylation sites to the antibody is conveniently accomplished by altering the amino acid sequence such that it contains one or more of the above-described tripeptide sequences (for N-linked glycosylation sites). The alteration may also be made by the addition of, or substitution by, one or more serine or threonine residues to the sequence of the original antibody (for O-linked glycosylation sites).

Nucleic acid molecules encoding amino acid sequence variants of the anti-LPA antibody are prepared by a variety of methods known in the art. These methods include, but are not limited to, isolation from a natural source (in the case of naturally occurring amino acid sequence variants) or preparation by oligonucleotide-mediated (or site-directed) mutagenesis, PCR mutagenesis, and cassette mutagenesis of an earlier prepared variant or a non-variant version of the anti-LPA antibody.

(v) Human Antibodies.

As an alternative to humanization, human antibodies can be generated. For example, it is now possible to produce transgenic animals (e.g., mice) that are capable, upon immunization, of producing a full repertoire of human antibodies in the absence of endogenous immunoglobulin production. For example, it has been described that the homozygous deletion of the antibody heavy-chain joining region (JH) gene in chimeric and germ-line mutant mice results in complete inhibition of endogenous antibody production. Transfer of the human germ-line immunoglobulin gene array in such germ-line mutant mice will result in the production of human antibodies upon antigen challenge. See, e.g., Jakobovits, et al., Proc. Natl. Acad. Sci. USA, 90:2551 (1993); Jakobovits, et al., Nature, 362:255-258 (1993); Bruggermann, et al., Year in Immuno., 7:33 (1993); and U.S. Pat. Nos. 5,591,669, 5,589,369 and 5,545,807. Human antibodies can also be derived from phage-display libraries (Hoogenboom, et al., J. Mol. Biol., 227:381 (1991); Marks, et al., J. Mol. Biol., 222:581-597 (1991); and U.S. Pat. Nos. 5,565,332 and 5,573,905). As discussed above, human antibodies may also be generated by in vitro activated B cells (see, e.g., U.S. Pat. Nos. 5,567,610 and 5,229,275) or by other suitable methods.

(vi) Antibody Fragments.

In certain embodiments, the humanized or variant anti-LPA antibody is an antibody fragment. Various techniques have been developed for the production of antibody fragments. Traditionally, these fragments were derived via proteolytic digestion of intact antibodies (see, e.g., Morimoto, et al., Journal of Biochemical and Biophysical Methods 24:107-117 (1992); and Brennan, et al., Science 229:81 (1985)). However, these fragments can now be produced directly by recombinant host cells. For example, Fab'-SH fragments can be directly recovered from E. coli and chemically coupled to form F(ab′)₂ fragments (Carter, et al., Bio/Technology 10:163-167 (1992)). In another embodiment, the F(ab′)₂ is formed using the leucine zipper GCN4 to promote assembly of the F(ab′)₂ molecule. According to another approach, Fv, Fab or F(ab′)₂ fragments can be isolated directly from recombinant host cell culture. Other techniques for the production of antibody fragments will be apparent to the skilled practitioner.

(vii) Multispecific Antibodies.

In some embodiments, it may be desirable to generate multispecific (e.g., bispecific) humanized or variant anti-LPA antibodies having binding specificities for at least two different epitopes. Exemplary bispecific antibodies may bind to two different epitopes of the lipid. Alternatively, an anti-LPA arm may be combined with an arm which binds to a different molecule. Bispecific antibodies can be prepared as full length antibodies or antibody fragments (e.g., F(ab′)₂ bispecific antibodies).

According to another approach for making bispecific antibodies, the interface between a pair of antibody molecules can be engineered to maximize the percentage of heterodimers that are recovered from recombinant cell culture. The preferred interface comprises at least a part of the C_{H}3 domain of an antibody constant domain. In this method, one or more small amino acid side chains from the interface of the first antibody molecule are replaced with larger side chains (e.g., tyrosine or tryptophan). Compensatory “cavities” of identical or similar size to the large side chain(s) are created on the interface of the second antibody molecule by replacing large amino acid side chains with smaller ones (e.g., alanine or threonine). This provides a mechanism for increasing the yield of the heterodimer over other unwanted end-products such as homodimers. See, e.g., U.S. Pat. No. 5,731,168.

Bispecific antibodies include cross-linked or “heteroconjugate” antibodies. For example, one of the antibodies in the heteroconjugate can be coupled to avidin, the other to biotin. Heteroconjugate antibodies may be made using any convenient cross-linking methods. Suitable cross-linking agents are well known in the art, and are disclosed in, for example, U.S. Pat. No. 4,676,980, along with a number of cross-linking techniques.

Techniques for generating bispecific antibodies from antibody fragments have also been described in the literature. For example, bispecific antibodies can be prepared using chemical linkage. Brennan, et al., Science 229:81 (1985) describe a procedure wherein intact antibodies are proteolytically cleaved to generate F(ab′)₂ fragments. These fragments are reduced in the presence of the dithiol complexing agent sodium arsenite to stabilize vicinal dithiols and prevent intermolecular disulfide formation. The Fab′ fragments generated are then converted to thionitrobenzoate (TNB) derivatives. One of the Fab′-TNB derivatives is then reconverted to the Fab′-thiol by reduction with mercaptoethylamine and is mixed with an equimolar amount of the other Fab′-TNB derivative to form the bispecific antibody. The bispecific antibodies produced can be used as agents for the selective immobilization of enzymes. In yet a further embodiment, Fab′-SH fragments directly recovered from E. coli can be chemically coupled in vitro to form bispecific antibodies. Shalaby, et al., J. Exp. Med. 175:217-225 (1992).

Various techniques for making and isolating bispecific antibody fragments directly from recombinant cell culture have also been described. For example, bispecific antibodies have been produced using leucine zippers. Kostelny, et al., J. Immunol. 148(5):1547-1553 (1992). The leucine zipper peptides from the Fos and Jun proteins were linked to the Fab′ portions of two different antibodies by gene fusion. The antibody homodimers were reduced at the hinge region to form monomers and then re-oxidized to form the antibody heterodimers. This method can also be utilized for the production of antibody homodimers. The “diabody” technology described by Hollinger, et al., Proc. Natl. Acad. Sci. USA 90:6444-6448 (1993) has provided an alternative mechanism for making bispecific antibody fragments. The fragments comprise a heavy-chain variable domain (V_{H}) connected to a light-chain variable domain (V_{L}) by a linker that is too short to allow pairing between the two domains on the same chain. Accordingly, the V_{H} and V_{L} domains of one fragment are forced to pair with the complementary V_{L} and V_{H} domains of another fragment, thereby forming two antigen-binding sites. Another strategy for making bispecific antibody fragments by the use of single-chain Fv (sFv) dimers has also been reported. See, e.g., Gruber, et al., J. Immunol. 152:5368 (1994). Alternatively, the bispecific antibody may be a “linear antibody” produced as described in, fror example, Zapata, et al. Protein Eng. 8(10):1057-1062 (1995).

Antibodies with more than two valencies are also contemplated. For example, trispecific antibodies can be prepared. Tutt et al., J. Immunol. 147:60 (1991).

An antibody (or polymer or polypeptide) of the invention comprising one or more binding sites per arm or fragment thereof will be referred to herein as “multivalent” antibody. For example a “bivalent” antibody of the invention comprises two binding sites per Fab or fragment thereof whereas a “trivalent” polypeptide of the invention comprises three binding sites per Fab or fragment thereof. In a multivalent polymer of the invention, the two or more binding sites per Fab may be binding to the same or different antigens. For example, the two or more binding sites in a multivalent polypeptide of the invention may be directed against the same antigen, for example against the same parts or epitopes of said antigen or against two or more same or different parts or epitopes of said antigen; and/or may be directed against different antigens; or a combination thereof. Thus, a bivalent polypeptide of the invention for example may comprise two identical binding sites, may comprise a first binding sites directed against a first part or epitope of an antigen and a second binding site directed against the same part or epitope of said antigen or against another part or epitope of said antigen; or may comprise a first binding sites directed against a first part or epitope of an antigen and a second binding site directed against the a different antigen. However, as will be clear from the description hereinabove, the invention is not limited thereto, in the sense that a multivalent polypeptide of the invention may comprise any number of binding sites directed against the same or different antigens.

An antibody (or polymer or polypeptide) of the invention that contains at least two binding sites per Fab or fragment thereof, in which at least one binding site is directed against a first antigen and a second binding site directed against a second antigen different from the first antigen, will also be referred to as “multispecific”. Thus, a “bispecific” polymer comprises at least one site directed against a first antigen and at least one a second site directed against a second antigen, whereas a “trispecific” is a polymer that comprises at least one binding site directed against a first antigen, at least one further binding site directed against a second antigen, and at least one further binding site directed against a third antigen, etc. Accordingly, in their simplest form, a bispecific polypeptide of the invention is a bivalent polypeptide (per Fab) of the invention. However, as will be clear from the description hereinabove, the invention is not limited thereto, in the sense that a multispecific polypeptide of the invention may comprise any number of binding sites directed against two or more different antigens.

(viii) Other Modifications.

Other modifications of the humanized or variant anti-LPA antibody are contemplated. For example, the invention also pertains to immunoconjugates comprising the antibody described herein conjugated to a cytotoxic agent such as a toxin (e.g., an enzymatically active toxin of bacterial, fungal, plant or animal origin, or fragments thereof), or a radioactive isotope (for example, a radioconjugate). Conjugates are made using a variety of bifunctional protein coupling agents such as N-succinimidyl-3-(2-pyridyldithiol) propionate (SPDP), iminothiolane (IT), bifunctional derivatives of imidoesters (such as dimethyl adipimidate HCL), active esters (such as disuccinimidyl suberate), aldehydes (such as glutaraldehyde), bis-azido compounds (such as bis (p-azidobenzoyl)hexanediamine), bis-diazonium derivatives (such as bis-(p-diazoniumbenzoyl)-ethylenediamine), diisocyanates (such as tolyene 2,6-diisocyanate), and bis-active fluorine compounds (such as 1,5-difluoro-2,4-dinitrobenzene).

The anti-LPA antibodies disclosed herein may also be formulated as immunoliposomes. Liposomes containing the antibody are prepared by methods known in the art, such as described in Epstein et al., Proc. Natl. Acad. Sci. USA 82:3688 (1985); Hwang, et al., Proc. Natl. Acad. Sci. USA 77:4030 (1980); and U.S. Pat. Nos. 4,485,045 and 4,544,545. Liposomes with enhanced circulation time are disclosed in U.S. Pat. No. 5,013,556. For example, liposomes can be generated by the reverse phase evaporation method with a lipid composition comprising phosphatidyl choline, cholesterol and PEG-derivatized phosphatidylethanolamine (PEG-PE). Liposomes are extruded through filters of defined pore size to yield liposomes with the desired diameter. Fab′ fragments of the antibody of the present invention can be conjugated to the liposomes as described in Martin, et al., J. Biol. Chem. 257:286-288 (1982) via a disulfide interchange reaction. Another active ingredient is optionally contained within the liposome.

Enzymes or other polypeptides can be covalently bound to the anti-LPA antibodies by techniques well known in the art such as the use of the heterobifunctional crosslinking reagents discussed above. Alternatively, fusion proteins comprising at least the antigen binding region of an antibody of the invention linked to at least a functionally active portion of an enzyme of the invention can be constructed using recombinant DNA techniques well known in the art (see, e.g., Neuberger, et al., Nature 312:604-608 (1984)).

It may be desirable to use an antibody fragment, rather than an intact antibody, to increase penetration of target tissues and cells, for example. In this case, it may be desirable to modify the antibody fragment in order to increase its serum half life. This may be achieved, for example, by incorporation of a salvage receptor binding epitope into the antibody fragment (e.g., by mutation of the appropriate region in the antibody fragment or by incorporating the epitope into a peptide tag that is then fused to the antibody fragment at either end or in the middle, e.g., by DNA or peptide synthesis). See, e.g., U.S. Pat. No. 6,096,871.

Covalent modifications of the humanized or variant anti-LPA antibody are also included within the scope of this invention. They may be made by chemical synthesis or by enzymatic or chemical cleavage of the antibody, if applicable. Other types of covalent modifications of the antibody are introduced into the molecule by reacting targeted amino acid residues of the antibody with an organic derivatizing agent that is capable of reacting with selected side chains or the N- or C-terminal residues. Exemplary covalent modifications of polypeptides are described in U.S. Pat. No. 5,534,615, specifically incorporated herein by reference. A preferred type of covalent modification of the antibody comprises linking the antibody to one of a variety of nonproteinaceous polymers, e.g., polyethylene glycol, polypropylene glycol, or polyoxyalkylenes, in the manner set forth in U.S. Pat. No. 4,640,835; 4,496,689; 4,301,144; 4,670,417; 4,791,192 or 4,179,337.

### B. Vectors, Host Cells and Recombinant Methods

The invention also provides isolated nucleic acid encoding the humanized or variant anti-LPA antibody, vectors and host cells comprising the nucleic acid, and recombinant techniques for the production of the antibody.

For recombinant production of the antibody, the nucleic acid encoding it may be isolated and inserted into a replicable vector for further cloning (amplification of the DNA) or for expression. In another embodiment, the antibody may be produced by homologous recombination, e.g., as described in U.S. Pat. No. 5,204,244. DNA encoding the monoclonal antibody is readily isolated and sequenced using conventional procedures (e.g., by using oligonucleotide probes that are capable of binding specifically to genes encoding the heavy and light chains of the antibody). Many vectors are available. The vector components generally include, but are not limited to, one or more of the following: a signal sequence, an origin of replication, one or more marker genes, an enhancer element, a promoter, and a transcription termination sequence, as described, for example, in U.S. Pat. No. 5,534,615.

Suitable host cells for cloning or expressing the DNA in the vectors herein are the prokaryote, yeast, or higher eukaryote cells described above. Suitable prokaryotes for this purpose include eubacteria, such as Gram-negative or Gram-positive organisms, for example, Enterobacteriaceae such as Escherichia, e.g., E. coli, Enterobacter, Erwinia, Klebsiella, Proteus, Salmonella, e.g., Salmonella typhimurium, Serratia, e.g., Serratia marcescans, and Shigella, as well as Bacilli such as B. subtilis and B. licheniformis (e.g., B. licheniformnis 41P), Pseudomonas such as P. aeruginosa, and Streptomyces. One preferred E. coli cloning host is E. coli 294 (ATCC 31,446), although other strains such as E. coli B, E. coli X1776 (ATCC 31,537), and E. coli W3110 (ATCC 27,325) are suitable. These examples are illustrative rather than limiting.

In addition to prokaryotes, eukaryotic microbes such as filamentous fungi or yeast are suitable cloning or expression hosts for anti-lysolipid antibody-encoding vectors. Saccharomyces cerevisiae, or common baker's yeast, is the most commonly used among lower eukaryotic host microorganisms. However, a number of other genera, species, and strains are commonly available and useful herein, such as Schizosaccharomyces pombe; Kluyveromyces hosts such as, e.g., K. lactis, K. fragilis (ATCC 12,424), K. bulgaricus (ATCC 16,045), K. wickeramii (ATCC 24,178), K. waltii (ATCC 56,500), K. drosophilarum (ATCC 36,906), K. thermotolerans, and K. marxianus; yarrowia (EP 402,226); Pichia pastoris (EP 183,070); Candida; Trichoderma reesia (EP 244,234); Neurospora crassa; Schwanniomyces such as Schwanniomyces occidentalis; and filamentous fungi such as, e.g., Neurospora, Penicillium, Tolypocladium, and Aspergillus hosts such as A. nidulans and A. niger.

Suitable host cells for the expression of glycosylated anti-LPA antibodies are derived from multicellularorganisms. Examples of invertebrate cells include plant and insect cells. Numerous baculoviral strains and variants and corresponding permissive insect host cells from hosts such as Spodoptera frugiperda (caterpillar), Aedes aegypti (mosquito), Aedes albopictus (mosquito), Drosophila melanogaster (fruitfly), and Bombyx mori have been identified. A variety of viral strains for transfection are publicly available, e.g., the L-1 variant of Autographa californica NPV and the Bm-5 strain of Bombyx mori NPV, and such viruses may be used as the virus herein according to the present invention, particularly for transfection of Spodoptera frugiperda cells. Plant cell cultures of cotton, corn, potato, soybean, petunia, tomato, and tobacco can also be utilized as hosts.

However, interest has been greatest in vertebrate cells, and propagation of vertebrate cells in culture (tissue culture) has become a routine procedure. Examples of useful mammalian host cell lines are monkey kidney CV1 line transformed by SV40 (COS-7, ATCC CRL 1651); human embryonic kidney line (293 or 293 cells subcloned for growth in suspension culture, Graham, et al., J. Gen Virol. 36:59 (1977)); baby hamster kidney cells (BHK, ATCC CCL 10); Chinese hamster ovary cells/-DHFR (CHO, Urlaub, et al., Proc. Natl. Acad. Sci. USA 77:4216 (1980)); mouse Sertoli cells (TM4, Mather, Biol. Reprod. 23:243-251 (1980)); monkey kidney cells (CV1 ATCC CCL 70); African green monkey kidney cells (VERO-76, ATCC CRL-1587); human cervical carcinoma cells (HELA, ATCC CCL 2); canine kidney cells (MDCK, ATCC CCL 34); buffalo rat liver cells (BRL 3A, ATCC CRL 1442); human lung cells (W138, ATCC CCL 75); human liver cells (Hep G2, HB 8065); mouse mammary tumor (MMT 060562, ATCC CCL51); TRI cells (Mather, et al., Annals N.Y. Acad. Sci. 383:44-68 (1982)); MRC 5 cells; FS4 cells; and a human hepatoma line (Hep G2).

Host cells are transformed with the above-described expression or cloning vectors for antibody production and cultured in conventional nutrient media modified as appropriate for inducing promoters, selecting transformants, or amplifying the genes encoding the desired sequences.

The host cells used to produce the antibody of this invention may be cultured in a variety of media. Commercially available media such as Ham's F10 (Sigma), Minimal Essential Medium ((MEM), (Sigma), RPMI-1640 (Sigma), and Dulbecco's Modified Eagle's Medium ((DMEM), Sigma) are suitable for culturing the host cells. In addition, any of the media described in Ham, et al., Meth. Enz. 58:44 (1979), Barnes, et al., Anal. Biochem. 102:255 (1980), U.S. Pat. No. 4,767,704; 4,657,866; 4,927,762; 4,560,655; or 5,122,469; WO 90/03430; WO 87/00195; or U.S. Pat. Re. 30,985 may be used as culture media for the host cells. Any of these media may be supplemented as necessary with hormones and/or other growth factors (such as insulin, transferrin, or epidermal growth factor), salts (such as sodium chloride, calcium, magnesium, and phosphate), buffers (such as HEPES), nucleotides (such as adenosine and thymidine), antibiotics (such as GENTAMYCIN™ drug), trace elements (defined as inorganic compounds usually present at final concentrations in the micromolar range), and glucose or an equivalent energy source. Any other necessary supplements may also be included at appropriate concentrations that would be known to those skilled in the art. The culture conditions, such as temperature, pH, and the like, are those previously used with the host cell selected for expression, and will be apparent to the ordinarily skilled artisan.

When using recombinant techniques, the antibody can be produced intracellularly, in the periplasmic space, or directly secreted into the medium. If the antibody is produced intracellularly, as a first step, the particulate debris, either host cells or lysed fragments, is removed, for example, by centrifugation or ultrafiltration. Carter, et al., Bio/Technology 10:163-167 (1992) describe a procedure for isolating antibodies that are secreted to the periplasmic space of E. coli. Briefly, cell paste is thawed in the presence of sodium acetate (pH 3.5), EDTA, and phenylmethylsulfonylfluoride (PMSF) over about 30 min. Cell debris can be removed by centrifugation. Where the antibody is secreted into the medium, supernatants from such expression systems are generally first concentrated using a commercially available protein concentration filter, for example, an Amicon or Millipore Pellicon ultrafiltration unit. A protease inhibitor such as PMSF may be included in any of the foregoing steps to inhibit proteolysis and antibiotics may be included to prevent the growth of adventitious contaminants.

The antibody composition prepared from the cells can be purified using, for example, hydroxylapatite chromatography, gel electrophoresis, dialysis, and affinity chromatography, with affinity chromatography being the preferred purification technique.

The suitability of protein A as an affinity ligand depends on the species and isotype of any immunoglobulin Fc domain that is present in the antibody. Protein A can be used to purify antibodies that are based on human heavy chains (Lindmark, et al., J. Immunol. Meth. 62:1-13 (1983)). Protein G is recommended for all mouse isotypes and for human γ3 (Guss, et al., EMBO J. 5:15671575 (1986)). The matrix to which the affinity ligand is attached is most often agarose, but other matrices are available. Mechanically stable matrices such as controlled pore glass or poly(styrenedivinyl)benzene allow for faster flow rates and shorter processing times than can be achieved with agarose. Where the antibody comprises a C_{H3} domain, the Bakerbond ABX™ resin (J. T. Baker, Phillipsburg, N.J.) is useful for purification. Other techniques for protein purification, such as fractionation on an ion-exchange column, ethanol precipitation, Reverse Phase HPLC, chromatography on silica, chromatography on heparin SEPHAROSE™, chromatography on an anion or cation exchange resin (such as a polyaspartic acid column), chromatofocusing, SDS-PAGE, and ammonium sulfate precipitation are also available depending on the antibody to be recovered.

Following any preliminary purification step(s), the mixture comprising the antibody of interest and contaminants may be subjected to low pH hydrophobic interaction chromatography using an elution buffer at a pH between about 2.5-4.5, preferably performed at low salt concentrations (e.g., from about 0-0.25M salt).

### C. Pharmaceutical Formulations

Therapeutic formulations of an antibody or immune-derived moiety of the invention are prepared for storage by mixing the antibody having the desired degree of purity with optional physiologically acceptable carriers, excipients, or stabilizers (see, e.g., Remington's Pharmaceutical Sciences 16th edition, Osol, A. Ed. (1980)), in the form of lyophilized formulations or aqueous solutions. Acceptable carriers, excipients, or stabilizers are nontoxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEENT™, PLURONICS™ or polyethylene glycol (PEG).

The formulation herein may also contain more than one active compound as necessary for the particular indication being treated, preferably those with complementary activities that do not adversely affect each other. Such molecules are suitably present in combination in amounts that are effective for the purpose intended.

The active ingredients may also be entrapped in microcapsule prepared, for example, by coacervation techniques or by interfacial polymerization, for example, hydroxymethylcellulose or gelatin-microcapsule and poly-(methylmethacylate) microcapsule, respectively, in colloidal drug delivery systems (for example, liposomes, albumin microspheres, microemulsions, nano-particles and nanocapsules) or in macroemulsions. Such techniques are disclosed in Remington's Pharmaceutical Sciences, 16th edition, Osol, A. Ed. (1980). The formulations to be used for in vivo administration must be sterile. This is readily accomplished for instance by filtration through sterile filtration membranes.

Sustained-release preparations may be prepared. Suitable examples of sustained-release preparations include semipermeable matrices of solid hydrophobic polymers containing the antibody, which matrices are in the form of shaped articles, e.g., films, or microcapsule. Examples of sustained-release matrices include polyesters, hydrogels (for example, poly(2-hydroxyethyl-methacrylate), or poly(vinyl alcohol)), polylactides (U.S. Pat. No. 3,773,919), copolymers of L-glutamic acid and y-ethyl-L-glutamate, non-degradable ethylene-vinyl acetate, degradable lactic acid-glycolic acid copolymers such as the Lupron Depot™ (injectable microspheres composed of lactic acid-glycolic acid copolymer and leuprolide acetate), and poly-D-(−)-3-hydroxybutyric acid. While polymers such as ethylene-vinyl acetate and lactic acid-glycolic acid enable release of molecules for over 100 days, certain hydrogels release proteins for shorter time periods. When encapsulated antibodies remain in the body for a long time, they may denature or aggregate as a result of exposure to moisture at 37° C., resulting in a loss of biological activity and possible changes in immunogenicity. Rational strategies can be devised for stabilization depending on the mechanism involved. For example, if the aggregation mechanism is discovered to be intermolecular S—S bond formation through thio-disulfide interchange, stabilization may be achieved by modifying sulfhydryl residues, lyophilizing from acidic solutions, controlling moisture content, using appropriate additives, and developing specific polymer matrix compositions.

### D. Non-Therapeutic Uses for the Antibodies

Antibodies to bioactive lipids may be used as affinity purification agents. In this process, the antibodies are immobilized on a solid phase such a Sephadex resin or filter paper, using methods well known in the art. The immobilized antibody is contacted with a sample containing the lysolipid to be purified, and thereafter the support is washed with a suitable solvent that will remove substantially all the material in the sample except the lysolipid, which is bound to the immobilized antibody. Finally, the support is washed with another suitable solvent, such as glycine buffer, for instance between pH 3 to pH 5.0, that will release the lipid from the antibody.

Anti-lipid antibodies may also be useful in diagnostic assays for the target lipid, e.g., detecting its expression in specific cells, tissues (such as biopsy samples), or bodily fluids. Such diagnostic methods may be useful in diagnosis of a cardiovascular or cerebrovascular disease or disorder.

For diagnostic applications, the antibody typically will be labeled with a detectable moiety. Numerous labels are available which can be generally grouped into the following categories:

(a) Radioisotopes, such as ³⁵S, ¹⁴C, ¹²⁵I, ³H, and ¹³¹I. The antibody can be labeled with the radioisotope using the techniques described in Current Protocols in Immunology, Volumes 1 and 2, Coligen et al., Ed. Wiley-Interscience, New York, N.Y., Pubs. (1991), for example, and radioactivity can be measured using scintillation counting.

(b) Fluorescent labels such as rare earth chelates (europium chelates) or fluorescein and its derivatives, rhodamine and its derivatives, dansyl, Lissamine, phycoerythrin and Texas Red are available. The fluorescent labels can be conjugated to the antibody using the techniques disclosed in Current Protocols in Immunology, supra, for example. Fluorescence can be quantified using a fluorimeter.

(c) Various enzyme-substrate labels are available. For example, U.S. Pat. No. 4,275,149 provides a review of some of these. The enzyme generally catalyzes a chemical alteration of the chromogenic substrate that can be measured using various techniques. For example, the enzyme may catalyze a color change in a substrate, which can be measured spectrophotometrically. Alternatively, the enzyme may alter the fluorescence or chemiluminescence of the substrate. Techniques for quantifying a change in fluorescence are described above. The chemiluminescent substrate becomes electronically excited by a chemical reaction and may then emit light that can be measured (using a chemiluminometer, for example) or donates energy to a fluorescent acceptor. Examples of enzymatic labels include luciferases (e.g., firefly luciferase and bacterial luciferase; U.S. Pat. No. 4,737,456), luciferin, 2,3-dihydrophthalazinediones, malate dehydrogenase, urease, peroxidase such as horseradish peroxidase (HRPO), alkaline phosphatase, beta-galactosidase, glucoamylase, lysozyme, saccharide oxidases (e.g., glucose oxidase, galactose oxidase, and glucose-6-phosphate dehydrogenase), heterocyclicoxidases (such as uricase and xanthine oxidase), lactoperoxidase, microperoxidase, and the like. Techniques for conjugating enzymes to antibodies are described in O'Sullivan, et al., Methods for the Preparation of Enzyme-Antibody Conjugates for use in Enzyme Immunoassay, in Methods in Enzym. (ed J. Langone & H. Van Vunakis), Academic press, New York, 73:147-166 (1981).

### Examples of enzyme-substrate combinations include, for example

(i) Horseradish peroxidase (HRPO) with hydrogen peroxidase as a substrate, wherein the hydrogen peroxidase oxidizes a dye precursor (e.g., orthophenylene diamine (OPD) or 3,3,5,5′-tetramethyl benzidine hydrochloride (TMB));

(ii) alkaline phosphatase (AP) with para-Nitrophenyl phosphate as chromogenic substrate; and (iii) β-D-galactosidase (β-D-Gal) with a chromogenic substrate (e.g., p-nitrophenyl-β-D-galactosidase) or fluorogenic substrate 4-methylumbelliferyl-β-D-galactosidase.

Numerous other enzyme-substrate combinations are available to those skilled in the art. For a general review of these, see U.S. Pat. Nos. 4,275,149 and 4,318,980.

Sometimes, the label is indirectly conjugated with the antibody. The skilled artisan will be aware of various techniques for achieving this. For example, the antibody can be conjugated with biotin and any of the three broad categories of labels mentioned above can be conjugated with avidin, or vice versa. Biotin binds selectively to avidin and thus, the label can be conjugated with the antibody in this indirect manner. Alternatively, to achieve indirect conjugation of the label with the antibody, the antibody is conjugated with a small hapten (e.g., digoxin) and one of the different types of labels mentioned above is conjugated with an anti-hapten antibody (e.g., anti-digoxin antibody). Thus, indirect conjugation of the label with the antibody can be achieved.

In another embodiment of the invention, the antibody need not be labeled, and the presence thereof can be detected using a labeled secondary antibody which binds to the anti-lipid antibody.

The antibodies of the present invention may be employed in any known assay method, such as competitive binding assays, direct and indirect sandwich assays, and immunoprecipitation assays. See, e.g., Zola, Monoclonal Antibodies: A Manual of Techniques, pp. 147-158 (CRC Press, Inc. 1987).

Competitive binding assays rely on the ability of a labeled standard to compete with the test sample analyte for binding with a limited amount of antibody. The amount of bioactive lipid in the test sample is inversely proportional to the amount of standard that becomes bound to the antibodies. To facilitate determining the amount of standard that becomes bound, the antibodies generally are insoluble before or after the competition, so that the standard and analyte that are bound to the antibodies may conveniently be separated from the standard and analyte that remain unbound.

Sandwich assays involve the use of two antibodies, each capable of binding to a different immunogenic portion, or epitope, of the protein to be detected. In a sandwich assay, the test sample analyte is bound by a first antibody that is immobilized on a solid support, and thereafter a second antibody binds to the analyte, thus forming an insoluble three-part complex. See, e.g., U.S. Pat. No. 4,376,110. The second antibody may itself be labeled with a detectable moiety (direct sandwich assays) or may be measured using an anti-immunoglobulin antibody that is labeled with a detectable moiety (indirect sandwich assay). For example, one type of sandwich assay is an ELISA assay, in which case the detectable moiety is an enzyme.

For immunohistochemistry, the blood or tissue sample may be fresh or frozen or may be embedded in paraffin and fixed with a preservative such as formalin, for example.

The antibodies may also be used for in vivo diagnostic assays. Generally, the antibody is labeled with a radionuclide (such as ¹¹¹In, ⁹⁹Tc, ¹⁴C, ¹³¹I, ¹²⁵I, ³H, ³²P, or ³⁵S) so that the bound target molecule can be localized using immunoscintillography.

### E. Diagnostic Kits

As a matter of convenience, antibodies to bioactive lipids can be provided in a kit, for example, a packaged combination of reagents in predetermined amounts with instructions for performing the diagnostic assay. Where the antibody is labeled with an enzyme, the kit will include substrates and cofactors required by the enzyme (e.g., a substrate precursor which provides the detectable chromophore or fluorophore). In addition, other additives may be included such as stabilizers, buffers (e.g., a block buffer or lysis buffer) and the like. The relative amounts of the various reagents may be varied widely to provide for concentrations in solution of the reagents which substantially optimize the sensitivity of the assay. Particularly, the reagents may be provided as dry powders, usually lyophilized, including excipients which on dissolution will provide a reagent solution having the appropriate concentration.

### F. Therapeutic Uses for the Antibody

For therapeutic applications, antibodies to bioactive lipids are administered to a mammal, preferably a human, in a pharmaceutically acceptable dosage form such as those discussed above, including those that may be administered to a human intravenously as a bolus or by continuous infusion over a period of time, by intramuscular, intraperitoneal, intra-cerebrospinal, subcutaneous, intra-articular, intrasynovial, intrathecal, oral, topical, or inhalation routes. For the prevention or treatment of disease, the appropriate dosage of antibody will depend on the type of disease to be treated, as defined above, the severity and course of the disease, whether the antibody is administered for preventive or therapeutic purposes, previous therapy, the patient's clinical history and response to the antibody, and the discretion of the attending physician. The antibody is suitably administered to the patient at one time or over a series of treatments.

Depending on the type and severity of the disease, about 1 ug/kg to about 50 mg/kg (e.g., 0.1-20 mg/kg) of antibody is an initial candidate dosage for administration to the patient, whether, for example, by one or more separate administrations, or by continuous infusion. A typical daily or weekly dosage might range from about 1 μg/kg to about 20 mg/kg or more, depending on the factors mentioned above. For repeated administrations over several days or longer, depending on the condition, the treatment is repeated until a desired suppression of disease symptoms occurs. However, other dosage regimens may be useful. The progress of this therapy is easily monitored by conventional techniques and assays, including, for example, radiographic imaging.

According to another embodiment of the invention, the effectiveness of the antibody in preventing or treating disease may be improved by administering the antibody serially or in combination with another agent that is effective for those purposes, such as chemotherapeutic anti-cancer drugs, for example. Such other agents may be present in the composition being administered or may be administered separately. The antibody is suitably administered serially or in combination with the other agent.

### G. Articles of Manufacture

In another embodiment of the invention, an article of manufacture containing materials useful for the treatment of the disorders described above is provided. The article of manufacture comprises a container and a label. Suitable containers include, for example, bottles, vials, syringes, and test tubes. The containers may be formed from a variety of materials such as glass or plastic. The container holds a composition which is effective for treating the condition and may have a sterile access port (for example the container may be an intravenous solution bag or a vial having a stopper pierceable by a hypodermic injection needle). The active agent in the composition is the anti-lysolipid antibody. The label on, or associated with, the container indicates that the composition is used for treating the condition of choice. The article of manufacture may further comprise a second container comprising a pharmaceutically-acceptable buffer, such as phosphate-buffered saline, Ringer's solution and dextrose solution. It may further include other materials desirable from a commercial and user standpoint, including other buffers, diluents, filters, needles, syringes, and package inserts with instructions for use.

### H. Structure-Based Design of Humanized Monoclonal Antibodies to Recognize Bioactive Lipids: Platform for Drug Discovery

Lpath's proprietary Immune Y2™ technology allows the generation of monoclonal antibodies against bioactive lipids, including LPA. Lpath's mAbs Sonepcizumab and Lpathomab (also referred to as LT1009 and LT3015, humanized monoclonal antibodies targeted to S1P and LPA, respectively) are first-in-class examples of antibody drugs against bioactive lipids.

Because of similarities in the structural framework of LT1009 and LT3015, and aided by recently derived x-ray diffraction data on LT1009 Fab fragment-S1P co-crystals, it is believed that in silico modeling can be used to generate new mAbs against different bioactive lipid targets without the need to immunize mice. This is facilitated by the relatively small sequence/structure space of lysolipids and similar bioactive lipids compared to that of proteinaceous antigens. It is believed that the expensive and complicated process of humanization can also be avoided by using this in silico method. It is proposed to use structure activity relationship (SAR) assays unique to the Immune Y2 platform to make mutations in the humanized framework and CDRs of LT3015 and/or LT1009, to alter their affinity and/or specificity for their respective ligands. Ultimately it is believed that mutations can be made to alter the specificity to such a point that the binding specificity of the antibody can be converted to a different ligand entirely; e.g., from LPA or S1P (depending on whether LT3015 or LT1009 was the starting point) to another bioactive lipid.

The invention will be better understood by reference to the following Examples, which are intended to merely illustrate the best mode now known for practicing the invention. The scope of the invention is not to be considered limited thereto.

### EXAMPLES

### Example 1

### Production and Characterization of Monoclonal Antibodies to LPA

Antibody Production

Although polyclonal antibodies against naturally-occurring LPA have been reported in the literature (Chen J H, et al., Bioorg Med Chem Lett. 2000 Aug. 7:10(15):1691-3), monoclonal antibodies had not been described until Lpath, Inc. developed a series of monoclonal antibodies to LPA, as described, for example, in U.S. application Ser. No. 11/755,721 (published as US 20080145360; attorney docket no. LPT-3100-UT4), and U.S. application Ser. No. 12/129,109 (published as US20090136483; attorney docket no. LPT-3200-UT), the contents of which are incorporated herein in their entirety and for all purposes. A C-12 thio-LPA analog was the key component of a hapten formed by the cross-linking of the analog via the reactive SH group to a protein carrier (KLH) via standard chemical cross-linking using either IOA or SMCC as the cross-linking agent. Mice were immunized with the thio-LPA-KLH hapten (in this case, thiolated-LPA:SMCC:KLH) for the generation of anti-S1P monoclonal antibodies. Of the 80 mice immunized against the LPA analog, the five animals that showed the highest titers against LPA (determined using an ELISA in which the same LPA analog used in the hapten was conjugated to BSA using SMCC and laid down on the ELISA plates) were chosen for moving to the hybridoma phase of development.

The spleens from these five mice were harvested and hybridomas were generated by standard techniques. Briefly, one mouse yielded hybridoma cell lines (designated 504A). Of all the plated hybridomas of the 504A series, 66 showed positive antibody production as measured by screening ELISA.

Table 2, below, shows the antibody titers in cell supernatants of hybridomas created from the spleens of two of mice that responded to an LPA analog hapten in which the thiolated LPA analog was cross-linked to KLH using heterobifunctional cross-linking agents. These data demonstrate that the anti-LPA antibodies do not react either to the crosslinker or to the protein carrier. Importantly, the data show that the hybridomas produce antibodies against LPA, and not against S1P.

TABLE 2
LPA hybridomas

| | 3rd bleed | | LPA | S1P | |
|---|---|---|---|---|---|
| | titer | | binding | binding | |
| | OD at | Supernatants | OD at | OD at | Cross reactivity |
| mouse # | 1:312,500 | from 24 well | 1:20 | 1:20 | w/ S1P* |
| 1 | 1.242 | 1.A.63 | 1.197 | 0.231 | low |
| | | 1.A.65 | 1.545 | 0.176 | none |
| 2 | 0.709 | 2.B.7 | 2.357 | 0.302 | low |
| | | 2.B.63 | 2.302 | 0.229 | low |
| | | 2.B.83 | 2.712 | 0.175 | none |
| | | 2.B.104 | 2.57 | 0.164 | none |
| | | 2.B.IB7 | 2.387 | 0.163 | none |
| | | 2.B.3A6 | 2.227 | 0.134 | none |

*Cross reactivity with S1P from 24 well supernatants high = OD > 1.0-2.0 at [1:20] mid = OD 0.4-1.0 at [1:20] low = OD 0.4-0.2 at [1:20] none = OD < 0.2 OD at [1:20]

The development of anti-LPA mAbs in mice was monitored by ELISA (direct binding to 12:0 and 18:0 LPA and competition ELISA). A significant immunological response was observed in at least half of the immunized mice and five mice with the highest antibody titer were selected to initiate hybridoma cell line development following spleen fusion.

After the initial screening of over 2000 hybridoma cell lines generated from these 5 fusions, a total of 29 anti-LPA secreting hybridoma cell lines exhibited high binding to 18:0 LPA. Of these hybridoma cell lines, 24 were further subcloned and characterized in a panel of ELISA assays. From the 24 clones that remained positive, six hybridoma clones were selected for further characterization. Their selection was based on their superior biochemical and biological properties.

### Direct Binding Kinetics

The binding of 6 anti-LPA mAbs (B3, B7, B58, A63, B3A6, D22) to 12:0 and 18:0 LPA (0.1 uM) was measured by ELISA. EC₅₀ values were calculated from titration curves using 6 increasing concentrations of purified mAbs (0 to 0.4 ug/ml). EC50 represents the effective antibody concentration with 50% of the maximum binding. Max denotes the maximal binding (expressed as OD450). Results are shown in Table 3.

TABLE 3
Direct Binding Kinetics of Anti-LPA mAbs

| | | B3 | B7 | B58 | D22 | A63 | B3A6 |
|---|---|---|---|---|---|---|---|
| 12:0 LPA | EC₅₀ (nM) | 1.420 | 0.413 | 0.554 | 1.307 | 0.280 | 0.344 |
| | Max (OD450) | 1.809 | 1.395 | 1.352 | 0.449 | 1.269 | 1.316 |
| 18:0 LPA | EC₅₀ (nM) | 1.067 | 0.274 | 0.245 | 0.176 | 0.298 | 0.469 |
| | Max (OD450) | 1.264 | 0.973 | 0.847 | 0.353 | 1.302 | 1.027 |

The kinetics parameters kₐ (association rate constant), k_{d} (disassociation rate constant) and K_{D} (association equilibrium constant) were determined for the 6 lead candidates using the BIAcore 3000 Biosensor machine. In this study, LPA was immobilized on the sensor surface and the anti-LPA mAbs were flowed in solution across the surface. As shown, all six mAbs bound LPA with similar K_{D} values ranging from 0.34 to 3.8 pM and similar kinetic parameters.

The Anti-LPA Murine mAbs Exhibit High Affinity to LPA

LPA was immobilized to the sensor chip at densities ranging 150 resonance units. Dilutions of each mAb were passed over the immobilized LPA and kinetic constants were obtained by nonlinear regression of association/dissociation phases. Errors are given as the standard deviation using at least three determinations in duplicate runs. Apparent affinities were determined by K_{D}=kₐ/k_{d}. kₐ=Association rate constant in M⁻¹s⁻¹ k_{d}=Dissociation rate constant in s⁻¹

TABLE 4
Affinity of anti-LPA mAb for LPA

| mAbs | kₐ (M⁻¹ s⁻¹) | k_{d} (s⁻¹) | K_{D} (pM) |
|---|---|---|---|
| A63 | 4.4 ± 1.0 × 10⁵ | 1 × 10⁻⁶ | 2.3 ± 0.5 |
| B3 | 7.0 ± 1.5 × 10⁵ | 1 × 10⁻⁶ | 1.4 ± 0.3 |
| B7 | 6.2 ± 0.1 × 10⁵ | 1 × 10⁻⁶ | 1.6 ± 0.1 |
| D22 | 3.0 ± 0.9 × 10⁴ | 1 × 10⁻⁶ | 33 ± 10 |
| B3A6 | 1.2 ± 0.9 × 10⁶ | 1.9 ± 0.4 × 10⁻⁵ | 16 ± 1.2 |

Specificity Profile of Six Anti-LPA mAbs.

Many isoforms of LPA have been identified to be biologically active and it is preferable that the mAb recognize all of them to some extent to be of therapeutic relevance. The specificity of the anti-LPA mAbs was evaluated utilizing a competition assay in which the competitor lipid was added to the antibody-immobilized lipid mixture. Competition ELISA assays were performed with 6 mAbs to assess their specificity. 18:0 LPA was captured on ELISA plates. Each competitor lipid (up to 10 uM) was serially diluted in BSA (1 mg/ml)-PBS and then incubated with the mAbs (3 nM). Mixtures were then transferred to LPA coated wells and the amount of bound antibody was measured with a secondary antibody. Data are normalized to maximum signal (A450) and are expressed as percent inhibition. Assays were performed in triplicate. IC₅₀: Half maximum inhibition concentration; MI: Maximum inhibition (% of binding in the absence of inhibitor); ---: not estimated because of weak inhibition. A high inhibition result indicates recognition of the competitor lipid by the antibody. As shown in Table 5, all of the anti-LPA mAbs recognized the different LPA isoforms.

TABLE 5
Specificity profile of six anti-LPA mAbs.

| | 14:0 LPA | | 16:0 LPA | | 18:1 LPA | | 18:2 LPA | | 20:4 LPA | |
|---|---|---|---|---|---|---|---|---|---|---|
| | IC₅₀ | MI | IC₅₀ | MI | IC₅₀ | MI | IC₅₀ | MI | IC₅₀ | MI |
| | uM | % | uM | % | uM | % | uM | % | uM | % |
| 504B3 | 0.02 | 72.3 | 0.05 | 70.3 | 0.287 | 83 | 0.064 | 72.5 | 0.02 | 67.1 |
| 504B7 | 0.105 | 61.3 | 0.483 | 62.9 | >2.0 | 100 | 1.487 | 100 | 0.161 | 67 |
| 504B58-3F8 | 0.26 | 63.9 | 5.698 | >100 | 1.5 | 79.3 | 1.240 | 92.6 | 0.304 | 79.8 |
| 504B104 | 0.32 | 23.1 | 1.557 | 26.5 | 28.648 | >100 | 1.591 | 36 | 0.32 | 20.1 |
| 504D22-1 | 0.164 | 34.9 | 0.543 | 31 | 1.489 | 47.7 | 0.331 | 31.4 | 0.164 | 29.5 |
| 504A63-1 | 1.147 | 31.9 | 5.994 | 45.7 | — | — | — | — | 0.119 | 14.5 |
| 504B3A6-1 | 0.108 | 59.9 | 1.151 | 81.1 | 1.897 | 87.6 | — | — | 0.131 | 44.9 |

Interestingly, the anti-LPA mAbs were able to discriminate between 12:0 (lauroyl), 14:0 (myristoyl), 16:0 (palmitoyl), 18:1 (oleoyl), 18:2 (linoleoyl) and 20:4 (arachidonoyl) LPAs. The rank order for EC50 was for the unsaturated 18:2>18:1>20:4 and for the saturated lipids 14:0>16:0>18:0. mAbs with high specificity are desirable for ultimate drug development. The specificity of the anti-LPA mAbs was assessed for their binding to LPA related biolipids such as distearoyl-phosphatidic acid, lysophosphatidylcholine, S1P, ceramide and ceramide-1-phosphate. None of the six antibodies demonstrated cross-reactivity to distearoyl PA and LPC, the immediate metabolic precursor of LPA.

Antibody B7 was chosen for further study and was designated Lpathomab™ or LT3000.

### Example 2

### Lpathomab™ in Cancer and Andocienesis Models

The pleiotropic effects of LPA suggest that reduced availability (effective concentration) of extracellular LPA will (i) reduce growth, metastasis and angiogenesis of primary tumors and (ii) counter-act LPA's protective anti-apoptotic effect on tumor. Because of Lpathomab™/LT3000's potent and specific binding to LPA, we hypothesized that in vivo treatment of LT3000 in preclinical models of cancer would result in various therapeutic benefits. Additional descriptions of anti-cancer and other activities of several anti-LPA monoclonal antibodies are found in U.S. patent application Ser. Nos. 11/755,721, filed 30 May 2007, (PG Pub 20080145360, attorney docket no. LPT-3100-UT4), 12/129,109, filed 29 May 2008 (PGPub 20090136483, attorney docket no. LPT-3200-UT) and 12/406,874, filed 18 Mar. 2009, (US20100034814, Attorney Docket No. LPT-3200-CP) which are hereby incorporated by reference in their entirety and for all purposes.

Preclinical studies were conducted using a variety of in vitro and in vivo systems, demonstrating that Lpathomab™/LT3000 (administered every 3 days at doses of 10-50 mg/kg) exhibits a profile of activity that is consistent with various mechanisms of action, including:

Inhibition of tumor growth in human tumor xenograft models in vivo;

Reduction in LPA-dependent cell proliferation and invasion of human tumor in vitro;

Reduction in angiogenesis, together with reductions in circulating levels of tumorigenic/angiogenic growth factors including IL6, IL8, GM-CSF, MMP2 in vivo;

Reduced metastatic potential; and

Neutralization of LPA-induced protection against tumor-cell death.

In In Vitro Models:

Reduced proliferation of OVCAR3 ovarian cancer cells;

Neutralization of LPA-induced release of IL-8 from Caki-1, IL-8 and IL-6 from SKOV3 (ovarian) tumor cells in vitro;

Mitigation of LPA's effects in protecting SKOV3 tumor cells from apoptosis (which suggests enhanced efficacy when used in combination with standard chemotherapeutic agents);

Inhibition of LPA-induced tumor cell migration and invasion from chemotherapeutic agents.

In In Vivo Models:

Inhibition of metastasis and progression of orthotopic and subcutaneous human tumors implanted in nude mice;

Reduction of tumor-associated angiogenesis in subcutaneous SKOV3 xenograft models and in prostate DU145 cancer cells;

Neutralization of bFGF- and VEGF-induced angiogenesis in the murine Matrigel plug assay;

Reduced choroidal neovascularization in a model of laser-induced injury of Bruch's membrane in the eye.

Anti-fibrosis activity in lung fibroblasts; and

Reduction of inflammation and fibrosis following bleomycin lung injury.

Examples showing biological activity of anti-LPA monoclonal antibodies can be found in U.S. patent application Ser. Nos. 11/755,721, filed 30 May 2007, (PG Pub 20080145360, attorney docket no. LPT-3100-UT4), 12/129,109, filed 29 May 2008 (PGPub 20090136483, attorney docket no. LPT-3200-UT) and 12/406,874, filed 18 Mar. 2009, (US20100034814, Attorney Docket No. LPT-3200-CP) which are hereby incorporated by reference in their entirety and for all purposes.

### Example 3

### Murine Anti-LPA Antibody Variable Domain Sequences

The variable domains of B7 and other murine anti-LPA antibodies were cloned and expressed as described in U.S. application Ser. No. 12/406,874, filed 18 Mar. 2009 (PGPub 20100034814, attorney docket no. LPT-3200-CP), the contents of which are incorporated herein by reference in their entirety. The amino acid sequences for the complementarity-determining regions of the mouse heavy and light variable domains of antibody B7 are shown in Table 6

TABLE 6
Mouse LPA CDR amino acid sequences of the
mouse V_{H} and V_{L} domains for clone B7 of
mouse anti-LPA monoclonal antibody

| CLONE | | CDR | SEQ ID NO: |
|---|---|---|---|
| | V_{H} CDR | | |
| B7 | GYGFINYLIE* | CDRH1 | 1 |
| B7 | LINPGSDYTNYNENFKG | CDRH2 | 2 |
| B7 | RFGYYGSGNYFDY | CDRH3 | 3 |
| | V_{L} CDR | | |
| B7 | TSGQSLVHINGNTYLH | CDRL1 | 4 |
| B7 | KVSNLFS | CDRL2 | 5 |
| B7 | SQSTHFPFT | CDRL3 | 6 |

*The CDRH1 defined according to Kabat is the 10-amino acid sequence shown The five-amino acid portion of the Kabat sequence shown in bold (NYLIE; SEQ ID NO: 7) is the canonical CDRH1 sequence.

The sequences of the B7 antibody variable domains are shown in Table 7.

TABLE 7
Clone B7 variable domain amino acid sequences without
leader sequence and cut sites

| Sequence | SEQ ID NO: |
|---|---|
| B7 Heavy Chain | |
| QVQLQQSGAELVRPGTSVKVSCKASGYGFINYLIEWIKQRPGQGLEWIGLINPGSDYTNYNENFK | 8 |
| GKATLTADKSSSTAYMHLSSLTSEDSAVYFCARRFGYYGSGNYFDYWGQGTTLTVSS | |
| B7 Light Chain | |
| DVVMTQTPLSLPVSLGDQASISCTSGQSLVHINGNTYLHWYLQKPGQSPKLLIYKVSNLFSGVPD | 9 |
| RFSGSGSGTDFTLKISRVEAEDLGVYFCSQSTHFPFTFGTGTKLEIK | |

### Example 4

### Humanization of Lpathomab (LT3000)

The present example describes the generation of humanized variants of LT3000 and their biochemical properties. A summary of these variants and properties is in Table 8.

TABLE 8
Summary of humanization data

| Anti-LPA mAb variants | | | BackMut# | | Yield | EC₅₀ | Tm | K_{D} (C12) | K_{D} (C18) |
|---|---|---|---|---|---|---|---|---|---|
| Variant | LC | HC | LC | HC | ug IgG1 | ng/ml | ° C. | pM | pM |
| LT 3000 | Murine | Murine | n/a | n/a | — | 268 | 67 | 27 (7) | 11 (5) |
| LT3010 | 510 | 610 | n/a | n/a | — | 448 | 71.5 | 58 (66) | 159 (151) |
| LT3011 | 502 | 603 | 3 | 3 | 172 | 675 | 71 | nd | nd |
| LT3012 | 502 | 604 | 3 | 4 | 128 | 326 | 67.5 | 132 (102) | 201 (170) |
| LT3013 | 506 | 603 | 1 | 3 | 242 | 1302 | 71.5 | nd | nd |
| LT3014 | 506 | 604 | 1 | 4 | 451 | 560 | 66 | 218 (76) | 370 (340) |
| LT3015 | 502 | 602 | 3 | 6 | 416 | 293 | 71.5 | 80 (94) | 58 (60) |
| LT3016 | 506 | 602 | 1 | 6 | 318 | 506 | 71.5 | 126 (87) | 126 (108) |

Materials

3,3′,5,5′-tetramethylbenzidine liquid substrate (TMB) was from Sigma-Aldrich (St. Louis, Mo.). Fatty acid-free bovine serum albumin (BSA) was from Calbiochem (La Jolla, Calif.). Immobilized Protein A, Immobilized Papain and protein desalting spin column were from Pierce (Rockford, Ill.). Anti-human IgG (Fc specific) antibody was purchased from Bethyl (Montgomery, Tex.). Reference IgGs (non-specific human IgG and mouse IgG), anti-human IgG (H+L)-horseradish peroxidase conjugate and anti-mouse IgG (H+L)-horseradish peroxidase conjugate were from Jackson ImmunoResearch Laboratories (West Grove, Pa.). Lysophosphatidic acid (LPA) and other lipids used in the competition ELISA were purchased from Avanti Polar Lipids (Alabaster, Ala.). Biotinylated LPA was purchased from Echelon Biosciences (Salt Lake City, Utah).

Humanization

The variable domains V_{H} and V_{L} of the murine anti-LPA monoclonal antibody, LT3000 (Lpathomab) were humanized by grafting the murine CDRs into human framework regions (FR), with the goal of producing an antibody that retains high affinity, specificity and binding capacity for LPA. Lefranc, M. P, (2003). Nucleic Acids Res, 31: 307-10; Martin, A. C. and J. M. Thornton, (1996) J Mol Biol, 1996. 263: 800-15; Morea, V., A. M. Lesk, and A. Tramontano (2000) Methods, 20: 267-79; Foote, J. and G. Winter, (1992) J Mol Biol, 224: 487-99; Chothia, C., et al., (1985). J Mol Biol, 186:651-63.

Suitable acceptor human FR sequences were selected from the IMGT and Kabat databases based on a homology to LT3000 using a sequence alignment and analysis program (SR v7.6). Lefranc, M. P. (2003) Nucl. Acids Res. 31:307-310; Kabat, E. A. et al. (1991) Sequences of Proteins of Immunological Interest, NIH National Techn. Inform. Service, pp. 1-3242. Sequences with high identity at FR, vernier, canonical and VH-VL interface residues (VCI) were initially selected. From this subset, sequences with the most non-conservative VCI substitutions, unusual proline or cysteine residues and somatic mutations were excluded. AJ002773 was thus selected as the human framework on which to base the humanized version of LT3000 heavy chain variable domain and DQ187679 was thus selected as the human framework on which to base the humanized version of LT3000 light chain variable domain.

A three-dimensional (3D) model containing the humanized VL and VH sequences was constructed to identify FR residues juxtaposed to residues that form the CDRs. These FR residues potentially influence the CDR loop structure and the ability of the antibody to retain high affinity and specificity for the antigen. Based on this analysis, 6 residues in AJ002773 and 3 residues in DQ187679 were identified, deemed significantly different from LT3000, and considered for mutation back to the LT3000 murine sequence. Framework selection and backmutation identification was conducted by DataMabs, LLP, Radlett, Hertfordshire, UK. A list of the humanized variants is summarized in Table 9. The 12V mutation, which is present within the light chain of every variant studied, supports the presentation of residues in the CDRL3. Other light chain back mutations include Q45K, which is solvent exposed, and the conservative Y87F mutation, located on the side of the variable domain opposite the CDRs. Based on their position, the heavy chain back mutations appear more likely to influence the stability and LPA-binding properties of the mAb. 124A and V28G support residues that form the CDRH1 and the cluster of back mutations (137V, M481, V67A and I69L) form an elaborate network of hydrophobic interactions that likely effect the stability of the folded variable domain and the position of the CDRH2. The role of these back mutations on LPA binding, thermostability and cytokine released were investigated to identify the lead candidate for development of a fully humanized, anti-LPA monoclonal antibody.

TABLE 9
Vector designation and expression level of the chimeric and
the humanized variants in HEK293 cells.

| | Light Chain | | Heavy Chain | | |
|---|---|---|---|---|---|
| | | Back | | Back | Expression |
| mAb | pATH | mutations | pATH | mutations | (ug/ml) |
| LT3010 | 510 | none | 610 | None | 8.44 |
| LT3011 | 502 | I2V, Q45K, | 603 | S24A, I28G, | 2.88 |
| | | Y87F | | M48I | |
| LT3012 | 502 | I2V, Q45K, | 604 | I28G, M48I, | 11.2 |
| | | Y87F | | V67A, I69L | |
| LT3013 | 506 | I2V | 603 | S24A, I28G, | 5.33 |
| | | | | M48I | |
| LT3014 | 506 | I2V | 604 | I28G, M48I, | 5.83 |
| | | | | V67A, I69L | |
| LT3015 | 502 | I2V, Q45K, | 602 | S24A, I28G, | 5.99 |
| | | Y87F | | V37I, M48I, | |
| | | | | V67A, I69L | |
| LT3016 | 506 | I2V | 602 | S24A, I28G, | 3.74 |
| | | | | V37I, M48I, | |
| | | | | V67A, I69L | |

A comparison of the variant sequences is shown in Tables 58 and 60. Backmutations are shown in bold. CDR sequences are shown in gray. Canonical residues are numbered according to the CDR (1, 2 or 3) which which they are associated.

TABLE 10
Sequences of the variable domains of anti-LPA light chain humanized variants. CDRs are shaded, backmutations are in bold.

| | VK sequence | SEQ ID NO: |
|---|---|---|
| Canonical | 1 1 1 1 1 2 2 1 3 3 3 | |
| Vernier | * * ** **** * * ** * * | |
| Interface | F F F F F F F F F | |
| Kabat number | | |
| B7 VK murine | 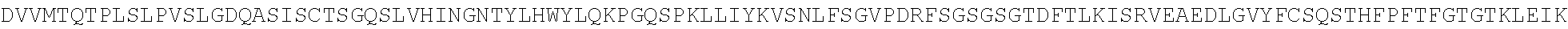 | 10 |
| B7RKA | 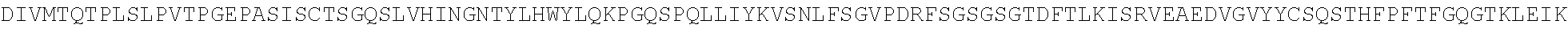 | 11 |
| B7RKB | 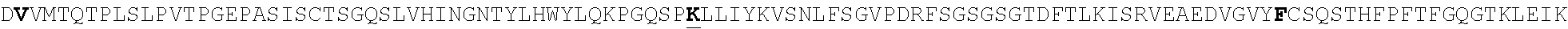 | 12 |
| B7RKC | 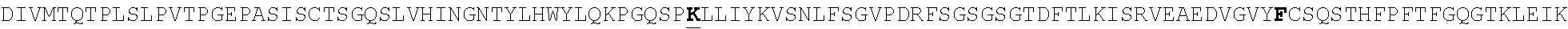 | 13 |
| B7RKD | 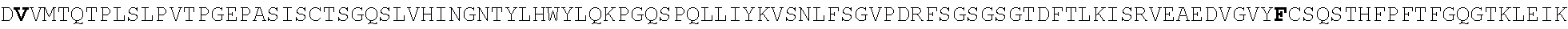 | 14 |
| B7RKE | 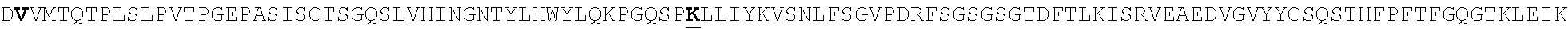 | 15 |
| B7RKF | 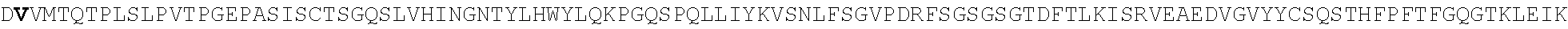 | 16 |
| B3-700 | 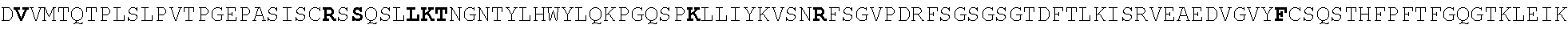 | 17 |
| B3-701 | 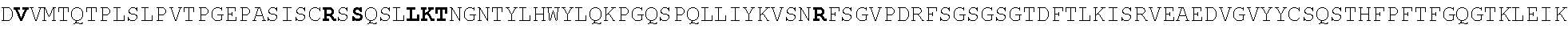 | 18 |
| B3-702 | 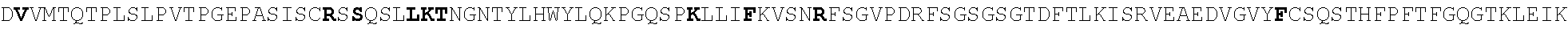 | 19 |

TABLE 11
LPA humanized antibody light chain variant variable domain
sequences and vectors containing them.

| | | Number of | Identity of |
|---|---|---|---|
| Vector name | Description | backmutations | backmutations |
| pATH500LC | pCONkappa | | |
| | (Lonza vector alone) | | |
| pATH501 | B7 humanized | 0 | — |
| | light chain RKA | | |
| | in vector pATH500LC | | |
| pATH502 | B7 humanized | 3 | I2V, Q45K, Y87F |
| | light chain RKB | | |
| | in vector pATH500 | | |
| pATH503 | B7 humanized | 2 | Q45K, Y87F |
| | light chain RKC | | |
| | in vector pATH500 | | |
| pATH504 | B7 humanized | 2 | I2V, Y87F |
| | light chain RKD | | |
| | in vector pATH500 | | |
| pATH505 | B7 humanized | 2 | I2V, Q45K |
| | light chain RKE | | |
| | in vector pATH500 | | |
| pATH506 | B7 humanized | 1 | I2V |
| | light chain RKF | | |
| | in vector pATH500 | | |
| pATH700 | B3 humanized | 9 | I2V, T24R, |
| | light chain B3-700 | | G26S, V27cL, |
| | in vector pATH500 | | H27dK, I27eT, |
| | | | Q45K, L54R, |
| | | | Y87F |
| pATH701 | B3 humanized | 7 | I2V, T24R, |
| | light chain B3-701 | | G26S, V27cL, |
| | in vector pATH500 | | H27dK, I27eT, |
| | | | L54R, |
| pATH702 | B3 humanized | 10 | I2V, T24R, |
| | light chain B3-702 | | G26S, V27cL, |
| | in vector pATH500 | | H27dK, I27eT, |
| | | | Q45K, Y49F, |
| | | | L54R, Y87F |

TABLE 12
Sequences of the variable domains of anti-LPA heavy chain humanized variants. CDRs are shaded, backmutations are in bold

| | | SEQ |
|---|---|---|
| | 1 2 3 4 5 6 7 8 9 10 11 | ID |
| Kabat # | 1234567890123456789012345678901234567890123456789012a345678901234567890123456789012abc345678901234567890abcde1234567890123 | NO: |
| Canonical | 1 11 1 1 2 22 2 1 | |
| Vernier | * **** *** * * * * * ** * | |
| Interface | I I I I I I I I I I | |
| B7 VH murine | 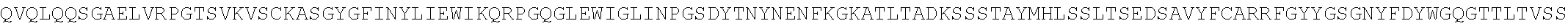 | 20 |
| B7RH0 | EVQLVQSGAEVKKPGESLKISCQSFGYIFINYLIEWVRQMPGQGLEWMGLINPGSDYTNYNENFKGQVTISADKSSSTAYLQWSSLKASDTAMYFCARRFGYYGSGNYFDYWGQGTMVTVSS | 21 |
| B7RH1 | 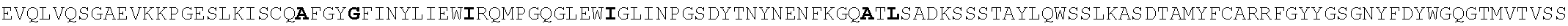 | 22 |
| B7RH2 | 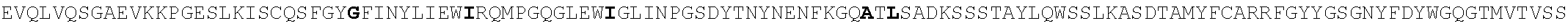 | 23 |
| B7RH3 | 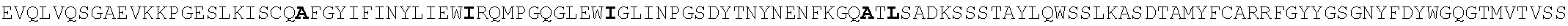 | 24 |
| B7RH4 | 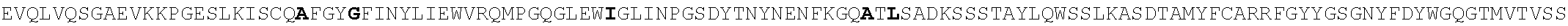 | 25 |
| B7RH5 | 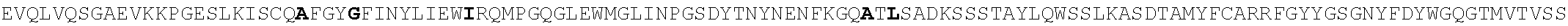 | 26 |
| B7RH6 | 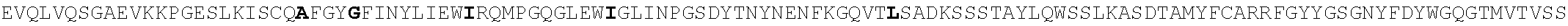 | 27 |
| B7RH7 | 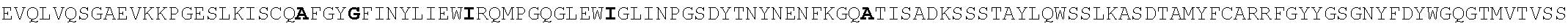 | 28 |
| B7RH8 | 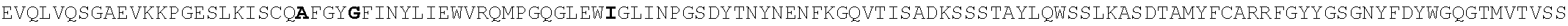 | 29 |
| B7RH9 | 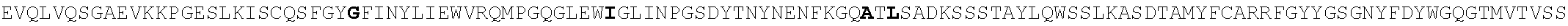 | 30 |
| B7HX | 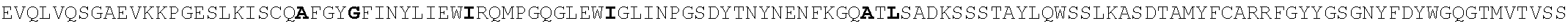 | 31 |
| B7HY | 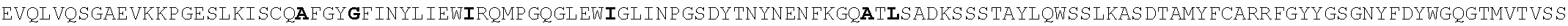 | 32 |
| B7HZ | 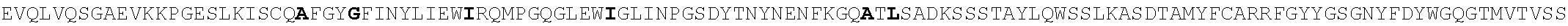 | 33 |
| B7-608 | 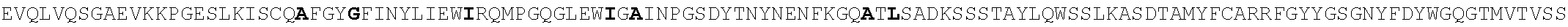 | 34 |
| B3-800 | 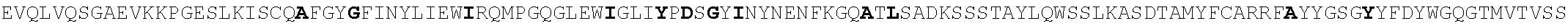 | 35 |
| B3-801 | 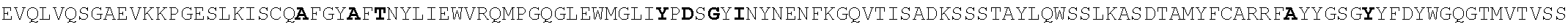 | 36 |
| B3-802 | 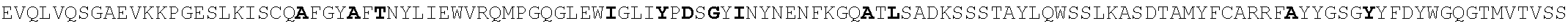 | 37 |
| B3-803 | 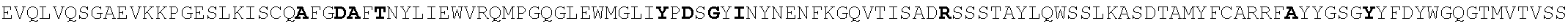 | 38 |
| B3-804 | 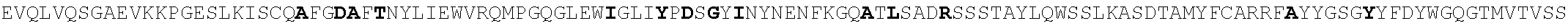 | 39 |

TABLE 13
LPA humanized antibody heavy chain variant variable
domain sequences and vectors containing them.

| | | Number of | |
|---|---|---|---|
| Vector name | Description | backmutations | Identity of backmutations |
| pATH600HC | pCONgamma (Lonza vector alone) | | |
| pATH601 | B7 humanized heavy chain RH0 in | 0 | — |
| | vector pATH600 | | |
| pATH602 | B7 humanized heavy chain RH1 in | 6 | S24A, I28G, V37I, M48I, V67A, |
| | vector pATH600 | | I69L |
| pATH603 | B7 humanized heavy chain RH8 in | 3 | S24A, I28G, M48I |
| | vector pATH600 | | |
| pATH604 | B7 humanized heavy chain RH9 in | 4 | I28G, M48I, V67A, I69L |
| | vector pATH600 | | |
| pATH605 | B7 humanized heavy chain HX in vector | 2 | I28G and M48I |
| | pATH600 | | |
| pATH606 | B7 humanized heavy chain HY in vector | 2 | S24A and M48I |
| | pATH600 | | |
| pATH607 | B7 humanized heavy chain HZ in vector | 4 | S24A, I28G, V37I, M48I |
| | pATH600 | | |
| pATH608 | B7 humanized heavy chain B7-608 in | 7 | S24A, I28G, V37I, M48I, L50A, |
| | vector pATH600 | | V67A, I69L, |
| pATH800 | B3 humanized heavy chain B3-800 in | 12 | S24A, I28G, V37I, M48I, N52Y, |
| | vector pATH600 | | G53D, D55G, T57I, V67A, I69L, |
| | | | G97A, N100cY |
| pATH801 | B3 humanized heavy chain B3-801 in | 9 | S24A, I28A, I30T, N52Y, G53D, |
| | vector pATH600 | | D55G, T57I, G97A, N100cY |
| pATH802 | B3 humanized heavy chain B3-802 in | 12 | S24A, I28A, I30T, M48I, N52Y, |
| | vector pATH600 | | G53D, D55G, T57I, V67A, |
| | | | I69L, G97A, N100cY |
| pATH803 | B3 humanized heavy chain B3-803 in | 11 | S24A, Y27D, I28A, I30T, N52Y, |
| | vector pATH600 | | G53D, D55G, T57I, K73R, G97A, |
| | | | N100cY |
| pATH804 | B3 humanized heavy chain B3-804 in | 14 | S24A, Y27D, I28A, I30T, M48I, |
| | vector pATH600 | | N52Y, G53D, D55G, T57I, V67A, |
| | | | I69L, K73R, G97A, N100cY |

### Expression of the Humanized Variants

The humanized variants shown in the table above were transiently expressed in HEK 293 cells in serum-free conditions, purified and then characterized in a panel of assays. Plasmids containing sequences of each light chain (pATH500 series) and heavy chain (pATH600 series) were transfected into mammalian cells for production. After 5 days of culture, the mAb titer was determined using quantitative ELISA. All combinations of the heavy and light chains yielded between 2-12 ug of antibody per ml of cell culture. SDS-PAGE under reducing conditions revealed two bands at 25 kDa and 50 kDa with high purity (>98%), consistent with the expected masses of the light and heavy chains. A single band was observed under non-reducing conditions with the expected mass of ˜150 KDa.

Characterization of the Humanized Variants

The biophysical properties of the humanized variants were characterized for their binding affinity, binding capacity, yield, potency and stability. All the humanized anti-LPA mAb variants exhibited binding affinity in the low picomolar range similar to the chimeric anti-LPA antibody (also known as LT3010) and the murine antibody (LT3000). All of the humanized variants exhibited a T_{M} similar to or higher than that of LT3000, and most had a Tm of approximately 71° C. With regard to specificity, the humanized variants demonstrated similar specificity profiles to that of LT3000. For example, LT3000 demonstrated no cross-reactivity to lysophosphatidyl choline (LPC), phosphatidic acid (PA), various isoforms of lysophosphatidic acid (14:0 and 18:1 LPA, cyclic phosphatidic acid (cPA), and phosphatidylcholine (PC).

Activity of the Humanized Variants

Five humanized variants (LT3011, LT3013, LT3014, LT3015 and LT3016) were further assessed in in vitro cell assays. LPA is known to play an important role in eliciting the release of interleukin-8 (IL-8) from cancer cells. LT3000 reduced IL-8 release from ovarian cancer cells in a concentration-dependent manner. The humanized variants exhibited a similar reduction of IL-8 release compared to LT3000.

Some humanized variants were also tested for their effect on microvessel density (MVD) in a Matrigel tube formation assay for neovascularization. Both were shown to decrease MVD formation.

TABLE 14
Quantitation of microblood vessel density using CD31 immunostain
with H&E counterstaining in matrigel plugs

| | | | | Human- | Human- | Human- |
|---|---|---|---|---|---|---|
| | | LT3000 | LT3000 | ized | ized | ized |
| | | murine | murine | variant #1 | variant #1 | variant #2 |
| | | (8 | (2 | (LT3015) | (LT3015) | (LT3016) |
| | Control | mg/kg) | mg/kg) | (8 mg/kg) | (2 mg/kg) | (2 mg/kg) |
| Average | 64.2 | 41.5 | 34 | 34.4 | 49 | 50.8 |
| S.E. | 8.0 | 14.2 | 13.7 | 4.2 | 31.5 | 18.8 |
| N = | 5 | 4 | 5 | 5 | 5 | 6 |

| | | | | | |
|---|---|---|---|---|---|
| Percent Inhibition | 35.4 | 47.0 | 46.4 | 23.7 | 20.8 |

Humanized anti-LPA antibody LT3015 was chosen for further characterization.

Antibody Expression and Production in Mammalian Cells The murine antibody genes were cloned from hybridomas. Synthetic genes containing the human framework

sequences and the murine CDRs were assembled from synthetic oligonucleotides and cloned into pCR4Blunt-TOPO using blunt restriction sites. After sequencing and observing 100% sequence congruence, the heavy and light chains were cloned and expressed as a full length IgG1 chimeric antibody using the pConGamma vector for the heavy chain gene and pConKappa vector for the light chain gene (Lonza Biologics, Portsmouth N.H.). The expression cassette for each of these genes contained a promoter, a kozak sequence, and a terminator. These plasmids were transformed into E. coli (One Shot Top 10 chemically competent E. coli cells, Invitrogen, Cat No. C4040-10), grown in LB media and stocked in glycerol. Large scale plasmid DNA was prepared as described by the manufacturer (Qiagen, endotoxin-free MAXIPREP™ kit, Cat. No 12362). Plasmids were transfected into the human embryonic kidney cell line 293F using 293fectin and using 293F-FreeStyle Media for culture. The transfected cultures expressed approximately 2-12 mg/L of humanized antibody.

Antibody Purification

Monoclonal antibodies were purified from culture supernatants using protein A affinity chromatography. Aliquots containing 0.5 ml of ProSep-vA-Ultra resin (Millipore, Cat. No 115115827) were added to gravity-flow disposable columns (Pierce, Cat. No 29924) and equilibrated with 10-15 ml of binding buffer (Pierce, Cat. No 21001). Culture supernatants containing transiently expressed humanized antibody were diluted 1:1 with binding buffer and passed over the resin. The antibody retained on the column was washed with 15 ml of binding buffer, eluted with low pH elution buffer (Pierce, Cat. No 21004) and collected in 1 ml fractions containing 100 ul of binding buffer to neutralize the pH. Fractions with absorbance (280 nm)>0.1 were dialyzed overnight (Slide-A-Lyzer Cassettes, 3500 MWCO, Pierce, Cat. No 66382) against 1 liter of PBS buffer (Cellgro, Cat. No 021-030). The dialyzed samples were concentrated using centricon-YM50 (Amicon, Cat. No 4225) concentrators and filtered through 0.22 uM cellulose acetate membranes (Costar, Cat. No 8160). The purity of each preparation was accessed using SDS-PAGE.

SDS-PAGE Electrophoresis

Each antibody sample was diluted to 0.5 ug/ul using gel loading buffer with (reduced) or without (non-reduced) 2-mercaptoethanol (Sigma, Cat. No M-3148). The reduced samples were heated at 95° C. for 5 min while the non-reduced samples were incubated at room temperature. A 4-12% gradient gel (Invitrogen, Cat. No NPO322) was loaded with 2 ug of antibody per lane and ran at 170 volts for 1 hour at room temperature in 1× NuPAGE MOPS SDS running buffer (Invitrogen, Cat. No NP0001). After electrophoresis, the antibodies were fixed by soaking the gel in 50% methanol, 10% acetic acid for ˜10 min. The gel was then washed with 3×200 ml distilled water. Finally, the bands were visualized by staining the gel overnight in GelCode® Blue Stain (Pierce, Cat. No 2490) and destaining with water.

Quantitative ELISA

The antibody titer was determined using a quantitative ELISA. Goat-anti human IgG-Fc antibody (Bethyl A80-104A, 1 mg/ml) was diluted 1:100 in carbonate buffer (100 mM NaHCO₃, 33.6 mM Na₂CO₃, pH 9.5). Plates were coated by incubating 100 ul/well of coating solution at 37° C. for 1 hour. The plates were washed 4× with TBS-T (50 mM Tris, 0.14 M NaCl, 0.05% tween-20, pH 8.0) and blocked with 200 ul/well TBS/BSA (50 mM Tris, 0.14 M NaCl, 1% BSA, pH 8.0) for 1 hour at 37° C. Samples and standard were prepared on non-binding plates with enough volume to run in duplicate. The standard was prepared by diluting human reference serum (Bethyl RS10-110; 4 mg/ml) in TBS-T/BSA (50 mM Tris, 0.14 NaCl, 1% BSA, 0.05% Tween-20, pH 8.0) to the following concentrations: 500 ng/ml, 250 ng/ml, 125 ng/ml, 62.5 ng/ml, 31.25 ng/ml, 15.625 ng/ml, 7.8125 ng/ml, and 0.0 ng/ml. Samples were prepared by making appropriate dilutions in TBS-T/BSA, such that the optical density (OD) of the samples fell within the range of the standard; the most linear range being from 125 ng/ml 15.625 ng/ml. After washing the plates 4× with TBS-T, 100 ul of the standard/samples preparation was added to each well and incubated at 37° C. for 1 hour. Next the plates were washed 4× with TBS-T and incubated for 1 hour at 37° C. with 100 ul/well of HRP-goat anti-human IgG antibody (Bethyl A80-104P, 1 mg/ml) diluted 1:150,000 in TBS-T/BSA. The plates were washed 4× with TBS-T and developed using 100 ul/well of TMB substrate chilled to 4° C. After 7 minutes, the reaction was stopped with 1M H2504 (100 ul/well). The OD was measured at 450 nm, and the data was analyzed using Graphpad Prizm software. The standard curve was fit using a four parameter equation and used to calculate the human IgG content in the samples.

Direct Binding ELISA

The LPA-binding affinities of the humanized antibodies were determined using a direct binding ELISA assay. Microtiter ELISA plates (Costar) were coated overnight with 1.0 ug/ml C12:0 LPA conjugated to Imject malieimide activated bovine serum albumin (BSA) (Pierce Co.) diluted in 0.1 M carbonate buffer (pH 9.5) at 37° C. for 1 h. Plates were washed with PBS (137 mM NaCl, 2.68 mM KCl, 10.1 mM Na₂HPO₄, 1.76 mM KH2PO₄; pH 7.4) and blocked with PBS/BSA/tween-20 for 1 hr at room temp or overnight at 4° C. For the primary incubation (1 hr at room temperature), a dilution series of the anti-LPA antibodies (0.4 ug/mL, 0.2 ug/mL, 0.1 ug/mL, 0.05 ug/mL, 0.0125 ug/mL, and 0 ug/mL) was added to the microplate (100 ml per well). Plates were washed and incubated with 100ul per well of HRP conjugated goat anti-human (H+L) diluted 1:20,000 (Jackson, cat #109-035-003) for 1 hr at room temperature. After washing, the peroxidase was developed with tetramethylbenzidine substrate (Sigma, cat No T0440) and stopped by adding 1 M H₂SO₄. The optical density (OD) was measured at 450 nm using a Thermo Multiskan EX. The EC₅₀ (half-maximal binding concentration) was determined by a least-squares fit of the dose-response curves with a four parameter equation using the Graphpad Prism software.

The EC₅₀ of the humanized antibody, LT3015, was determined to be 75.6 ng/mL, as compared to the murine antibody, LT3000, which had an EC50 of 65.3 ng/mL.

LPA Competition ELISA

The specificity of the humanized antibody was determined by competition ELISA. C18:0 LPA coating material was diluted to 0.33 ug/ml with carbonate buffer (100 mM NaHCO₃, 33.6 mM Na₂CO₃, pH 9.5). Plates were coated with 100 ul/well of coating solution and incubated at 37° C. for 1 hour. The plates were washed 4 times with PBS (100 mM Na₂HPO₄, 20 mM KH2PO₄, 27 mM KCl, 1.37 mM NaCl, pH 7.4) and blocked with 150 ul/well of PBS, 1% BSA, 0.1% tween-20 for 1 h at room temperature. The humanized, anti-LPA antibodies were tested against lipid competitors (14:0 LPA (Avanti, Cat. No 857120), 18:1 LPA (Avanti, Cat. No 857130), 18:1 LPC (Avanti, Cat. No 845875), cLPA (Avanti, Cat. No 857328), 18:1 PA (Avanti, Cat. No 840875), PC (Avanti, Cat. No 850454) at 5 uM, 2.5 uM, 1.25 uM, 0.625 uM, and 0.0 uM. The antibody was diluted to 0.5 ug/ml in PBS, 0.1% tween-20 and combined with the lipid samples at a 1:3 ratio of antibody to sample on a non-binding plate. The plates were washed 4 times with PBS and incubated for 1 hour at room temperature with 100 ul/well of the primary antibody/lipid complex. Next the plates were washed 4 times with PBS and incubated for 1 h at room temperature with 100 ul/well of HRP-conjugated goat anti-human antibody diluted 1:20,000 in PBS, 1% BSA, 0.1% tween-20. Again the plates were washed 4 times with PBS and developed using TMB substrate (100 ul/well) at 4° C. After 8 minutes, the reaction was stopped with 100 ul/well of 1M H2SO4. The optical density (OD) was measured at 450 nm using a Thermo Multiskan EX. Raw data were transferred to GraphPad software for analysis.

The IC₅₀ for the humanized mAb LT3015 was determined to be 0.08 uM, whereas the IC₅₀ for the corresponding murine antibody, LT3000, was 0.28 uM. Specificity and thermostability were also determined for LT3015 and this humanized mAb was found to retain the binding, specificity and thermostability of the murine parent antibody.

Measurement of LPA-Induced IL-6 and IL-8 Release in SKOV3 Cells

Anti-LPA antibodies inhibit the LPA-dependant release of human CXCL8/IL-8 in conditioned media of SKOV3 ovarian cells. SKOV3 cells (Lot No 4255558, passage 14) were harvested with 2 ml of 1× Trypsin EDTA (Mediatech Inc, Cat. No 25-053-CV) and resuspended in 8 ml of complete medium (10% FBS, Mediatech Inc. Cat. no 35-011-CV). The cells were centrifuged for 5 min (11,000 rpm) and re-suspended in 5 ml of complete medium. Cells were counted in duplicate with 0.4% Trypan blue (10 ul cells plus 90 ul Trypan blue, Invitrogen, Cat. No 15250-061) using a hemocytometer. In a 96-well plate, 1×10⁵ cells per well were seeded (final volume 100 ul/well). The cells were allowed to attach and form a confluent monolayer by incubating overnight at 37° C. On the following day, cells were gently washed two times with minimum media (1 mg/ml BSA in McCoy's medium with L-glutamine, Mediatech, Cat. No 10-050-CV). The media was adjusted to 1% penicillin/streptomycin (Mediatech, Cat. No 30-002 Cl) and 2.2 g/L sodium-bicarbonate (Mediatech, Cat. No 25-035-Cl). Next, the cells were serum-starved at 37° C. for exactly 24 h, followed by cytokine stimulation with 1 uM C18:1 LPA (Avanti, Cat. No 857130) dissolved in 1 mg/ml BSA/PBS (Calbiochem, Cat. No 126575) which was pre-incubated in presence or absence of humanized LPA antibody LT3015 (150, 300 or 600 ug/mL) for one hour. Treatments were then added to the cells. After 22 h of cytokine stimulation, the cells were centrifuged for 5 min (13,500 rpm) at 4° C. and the supernatants (cell-conditioned media) were collected. The CXCL8/IL-8 levels in each supernatant were measured using the Quantikine human CXCL8/IL-8 ELISA kit according to vendor instructions (R&D Systems, Minneapolis Minn., Cat. No D8000C). The IL-6 levels were measured by ELISA using the Quantikine human IL-6 immunoassay kit (R&D systems, Cat. No. D6050). Data were analyzed by one-way ANOVA followed by Bonferroni's post test and expressed as human IL-8 or human IL-6 fold increase. Data are shown in Table 15 and Table 16 below.

TABLE 15
Inhibition of human IL-8 release by humanized anti-LPA
antibody LT3015

| Stimulus condition | Human IL-8 Fold Increase (approx). |
|---|---|
| NT (no treatment) | 1 |
| 1 uM LPA | 7.1## |
| LPA + LT3015, 150 ug/mL | 5.7 |
| LPA + LT3015, 300 ug/mL | 4.5** |
| LPA + LT3015, 600 ug/mL | 2.7** |
| LT3015, 300 ug/mL | 1.1 |
| FBS (10%) | 20.1 |

(*p < 0.05, **p < 0.001 and ##p < 0.001, n = 3)

TABLE 16
Inhibition of human IL-6 release by humanized anti-LPA
antibody LT3015

| Stimulus condition | Human IL-6 Fold Increase (approx). |
|---|---|
| NT (no treatment) | 1 |
| 1 uM LPA | 29## |
| LPA + LT3015, 150 ug/mL | 22.1 |
| LPA + LT3015, 300 ug/mL | 15.7* |
| LPA + LT3015, 600 ug/mL | 10.8** |
| LT3015, 300 ug/mL | 1.1 |
| FBS (10%) | 69.2 |

(*p < 0.05, **p < 0.001 and ##p < 0.001, n = 3)

Measurement of Tumor Cell Migration in the Scratch Assay

SKOV3 cells were plated at 15,000 cells per well in a 96-well plate. The following day the cells were serum starved in minimal media (McCoy's Media 5a, adjusted to contain L-Glutamine, 2.2 g/L Sodium Bicarbonate, 1% penicillin/streptomycin and 1 mg/ml BSA) for 24 hrs. At time 0 cells were scratched with a p200 pipet tip down the center of each well, washed with minimal media and pictures were taken prior to treatment. Cells were then treated with LPA (C18:1) at 0.2 uM, 1.0 uM and 10 uM concentrations which were pre-incubated at 37° C. with 1.0 uM LPA in the presence or absence of antibody at 150 ug/ml. Positive control (10% FBS treated cells) and antibody alone added to 1 uM LPA, were also tested. Cells were stimulated for 17 hrs at 37° C. in a 5% CO₂ incubator. Pictures were taken again 17 hr post-treatment and % wound closure was measured by adjusting pictures to the same size and measuring the width of the scratch at time 0 and time 17 hr with a ruler. Data were analyzed by Student's t-test. Results are shown in Table 17:

TABLE 17
LT3015 prevents migration of ovarian cancer cells

| Treatment | Percent wound closure (approx.) |
|---|---|
| NT (no treatment) | 30 |
| 10% FBS | 98 |
| 0.2 uM LPA | 65 |
| 1.0 uM LPA | 81 |
| 10 uM LPA | 89 |
| LPA + LT3015 (150 ug/mL) | 59 |
| LT3015 (150 ug/mL) | 30 |

Reduction of Tumor Progression

Human mAb LT3015 reduced ovarian tumor SKOV3 progression and circulating cytokines in biological fluids. Nude mice were engrafted with either 10 mg/kg LT3015, vehicle, or 2 mg/kg paclitaxel (Taxol). After 56 days, mice were sacrificed and the peritoneal cavities were analyzed for tumor burden and ascites fluid accumulation. Tumors were harvested and final tumor weights were determined along with ascites volumes. Data were analyzed by ANOVA and student's t-test analysis. A 32% reduction in tumor burden was observed in LT3015-treated mice. Serum and ascites levels of IL-6, IL-8, GM-CSF and VEGF were measured using ELISA kits from R&D systems, Minneapolis Minn. (Cat. No. D6050, D8000C, HSGMO and DVE00, respectively) and a reduction in all was observed in LT3015-treated animals compared to vehicle controls, as shown in Table 18. *=p<0.05.

TABLE 18
LT3015 reduces SKOV3 tumor progression and
circulating cytokines in vivo

| | | LT3015 | Taxol |
|---|---|---|---|
| Analysis# | Vehicle | 10 mg/kg | 2 mg/kg |
| Tumor Burden | 1274 ± 209 | 861 ± 135 | 381 ± 73 |
| (mg) | | | |
| % Reduction | 100 | 32.4 | 70.1 |
| Ascites presence | 6/11 | 4/12 | 1/7 |
| Ascites volume | 3.563 ± 1.144 | 1.619 ± 0.6113 | 0.2 ± 0.0 |
| (mL) | | | |
| IL-8-serum | 687.6 ± 114.1 | 324.3 ± 55.23* | 234.7 ± 83.04* |
| (pg/mL) | | | |
| IL-6-serum | 84.56 ± 16.62 | 28.02 ± 6.212* | 29.19 ± 9.568* |
| (pg/mL) | | | |
| GM-CSF-serum | 320.5 ± 43.18 | 225.9 ± 54.15 | 340.2 ± 62.98 |
| (pg/mL) | | | |
| IL-8-ascites | 2097 ± 132.1 | 1292 ± 363.1 | 2187 ± 0## |
| (pg/mL) | | | |
| IL-6-ascites | 1018 ± 103.6 | 400.0 ± 218.9* | ND |
| (pg/mL) | | | |
| GM-CSF-ascites | 1200 ± 248.2 | 1289 ± 482.5* | 2054 ± 0## |
| (pg/mL) | | | |
| VEGF-ascites | 3341 ± 202.5 | 2697 ± 255.6 | 3344 ± 0## |
| (pg/mL) | | | |

| | |
|---|---|
| #Mean ± S.E. | 1 way ANOVA; Bonferroni's post test * p < 0.05 |
| | (Ascites IL-8 and IL-6, Student t test * p < 0.05) |

##Sample numbers for ascites measurements;
N = 6 (Vehicle),
N = 4 (hu-Ab), and
N = 1(Taxol).

### Example 5

### Isolation of Fab Fragments from Anti-LPA Monoclonal Antibody

Fab Preparation: apo Fab.

Purified, intact IgG was digested with activated papain (incubate 10 mg/mL papain in 5.5 mM cysteine-HCl, 1.1 mM EDTA, 67 μM 2-mercaptoethanol for 0.5 h at 37° C.) in digestion buffer (100:1 LT3015:papain in 50 mM sodium phosphate pH 7.2, 2 mM EDTA). After 3 h at 37° C. with slow rocking, the protease reaction was quenched with 50 mM iodoacetamide, diluted 6× in 50 mM Tris-HCl pH 7.5, 150 mM NaCl, and loaded onto a protein A packed column (Millipore) equilibrated with 50 mM Tris-HCl pH 7.5, 150 mM NaCl. The intact antibody and the Fc fragment bind to the resin, while the Fab fragment is present in the flow-through fraction. The purified Fab was concentrated at 4° C. using a centricon-YM30 centrifugal concentrator (Millipore) at 2000×G and loaded into a Superdex 75 10/300 size exclusion column equilibrated with 50 mM Tris-HCl pH 7.5, 150 mM NaCl. The fractions under the peak were collected, concentrated as before, and stored at 4° C.

Fab Preparation: Fab:LPA(14:0) & Fab:LPA(18:2) Complex.

Purified, intactlgG was digested with activated papain (incubate 10 mg/mL papain in 5.5 mM cysteine-HCl, 1.1 mM EDTA, 67 μM 2-mercaptoethanol for 0.5 h at 37° C.) in digestion buffer (100:1 LT3015:papain in 50 mM sodium phosphate pH 7.2, 2 mM EDTA). After 3 h at 37° C. with slow rocking, the protease reaction was quenched with 50 mM iodoacetamide, diluted 6× in ice chilled 50 mM Tris-HCl pH 7.5, 150 mM NaCl, and loaded onto 2 protein A packed columns (Millipore) linked in series and equilibrated with ice chilled 50 mM Tris-HCl pH 7.5, 150 mM NaCl. The intact antibody and the Fc fragment bind to the resin, while the Fab fragment is present in the flow through fraction. The purified Fab was concentrated at 4° C. using a centricon-YM30 centrifugal concentrator (Millipore) at 1500×G and loaded into an ice water jacket chilled Superdex 200 26/60 size exclusion column equilibrated with ice chilled 50 mM Tris-HCl pH 7.5, 150 mM NaCl. The fractions under the peak were collected, concentrated as before, and stored at 4° C.

The purified Fab was concentrated and was analyzed via an ELISA to determine if its LPA binding properties remained intact post digestion and purification. The ELISA revealed that the purified Fab maintained the ability to bind LPA comparably to full length undigested and unpurified LT3015.

### Example 6

### Generation of Fab and LPA-Fab Crystals

Antibody production. Stable CD-CHO cell lines that produce >0.5 mg/L of LT3015 have been developed by Lpath. While maintaining a viability of >95%, cells were seeded at a density of 0.4×106 cells/mL into 1 L shaker flasks with 0.5 L of CD-CHO media (Invitrogen) containing 25 mM L-methionine sulphoximine (Sigma). Cells were grown in 7.5% CO2 for 10 days or until viability reaches 45-50%. Supernatants were harvested by centrifugation at 1500 rpm for 10 minutes and sterile-filtered through a 0.22 mm filters (Corning). Clarified supernatants were concentrated ten-fold using a Labscale Tangential Flow Filtration system installed with a Pellicon XL Biomax 50 cartridge (Millipore). Filtrates were diluted with equal volume IgG binding buffer (Pierce) and applied to a column packed with ProSep-vA-Ultra resin (Millipore) equilibrated with binding buffer. The bound IgG was washed with binding buffer and eluted with elution buffer (Pierce) and collected in 40 mL fractions containing 5 mL of binding buffer to neutralize the pH. Fractions with an absorption at 280 nm (A280) greater than 0.1 were pooled, concentrated using an Amicon stirred cell equipped with a 50 kDa MWCO filter (Millipore). The concentrated antibody was extensively dialyzed against 1×PBS (Cellgro) and filtered through a 0.22 mM syringe-driven filter unit (Millipore) and stored at 4° C.

### Example 7

### Formation of the Antibody-Lipid Complexes

Complex Formation: Fab:LPA(14:0) & Fab:LPA(18:2). The final concentration of antibody Fab was determined by absorption at 280 nm. A 50 mg/mL LPA (Avanti Polar Lipids) suspension in chloroform/methanol/water was dried in a 12×75 mm borosilicate glass tube by holding it in a low vacuum for 1.5 hours. This LPA was resuspended to 50 mg/mL in 50 mM Tris-HCl pH 7.5, 150 mM NaCl. The solution was sonicated for 10 minutes, added to the Fab as a 10-fold molar excess, and incubated for 5 days at 4° C. The resulting 12 mg/mL Fab:LPA emulsion was filtered through 0.22 m

Costar Spin-X centrifugal cellulose acetate filter (Corning), transferred to a glass screw top vial and stored at 4° C.

### Example 8

### Crystallization

Crystallization: apo Fab. Crystals were grown at room temperature by the hanging drop, vapor diffusion method; 1 μL of 12 mg/mL Fab was mixed with 1 μL of reservoir solution containing 0.1 M HEPES sodium pH 7.5, 2% PEG 400 (v/v), and 1.75 M ammonium sulfate. The crystals grew to a final size of 0.1×0.1×0.1 mm in 3 days. Crystals were removed from the crystallization drop with nylon loops and immersed in a drop of mother liquor with 15% glycerol for 1 minute. They were then removed and

flash cooled directly in liquid nitrogen.

Crystallization: Fab:LPA(14:0) & Fab:LPA(18:2) Complex. Crystals were grown at room temperature by the hanging drop, vapor diffusion method; 1 μL of 12 mg/mL Fab was mixed with 1 μL of reservoir solution containing 0.095 M sodium citrate tribasic dihydrate, 19% (v/v) isopropanol, 19% (w/v) PEG 4000, and 5% (v/v) glycerol (Hampton Research). The crystals grew to a final size of 0.1×0.1×0.1 mm in 8 days. Crystals were removed from the crystallization drop with nylon loops and flash cooled directly in liquid nitrogen.

### Example 9

### Data Collection and Processing

Data Collection and Processing. X-ray diffraction data were collected at 100 K on an R-Axis IV image plate detector (Rigaku) at the San Diego State University Macromolecular x-ray Crystallography Facility. X-rays were produced by an RU-H3R rotating anode x-ray generator functioning at 100 mA and 50 kV with Osmic Blue confocal optics (Rigaku). Synchrotron data were collected on an ADSC Q315 CCD detector at the Advanced Light Source Beamline 8.2.2 Berkeley National Laboratory. Synchrotron data were also collected on an ADSC Q315 CCD detector at the National Synchrotron Light Source Beamline X25 Brookhaven National Laboratory. Data indexing and scaling were carried out using HKL2000. Otwinowski Z, Minor W (1997) Macromolecular Crystallography (Academic Press, San Diego Calif.).

Structure Solution, Model Building, and Refinement: apo Fab. Merged SCALEPACK output data were converted to MTZ format using scalepack2mtz. The CCP4 suite: programs for protein crystallography (1994). Acta Crystallogr. D. Biol. Crystallogr. 50:760-763. Molecular replacement was carried out in PHASER using LT1009 (PDB ID code 319G) as a starting model. McCoy, A. J. et al. (2007) J. Appl Crystallogr 40:658:674. For phasing this model was separated into constant and variable regions. Further refinement by maximum likelihood methods was run in REFMAC5. Murshudov G N, Vagin A A, Dodson E J (1997) Acta Crystallogr. D. Biol. Crystallogr. 53: 240-255. The model was rebuilt in the program COOT. Emsley, P. et al., (2010) Acta Crystallogr. D. Biol. Crystallogr. 66:486-501. The model then was then further built in the program PHENIX. Adams, P. D. et al. (2010) Acta Crystallogr. D. Biol. Crystallogr. 66:213:221. Coordinates for triethylene glycol (PGE) and sulfate ion were taken from the hic-up server. Kleywegt, G. J., T. A. Jones (1998) Acta. Crystallogr. D. Biol. Crystallogr. 54:1119-1131. A library file was prepared via Monomer Library Sketcher. The CCP4 suite: programs for protein crystallography (1994). Acta Crystallogr. D. Biol. Crystallogr. 50:760-763. Stereochemical analysis and final adjustments to the model were directed by MOLPROBITY. Davis I W et al., (2007) Nucl. Acids. Res. 35:W375-383. Final adjustments were made in COOT. Emsley, P. et al., (2010) Acta Crystallogr. D. Biol. Crystallogr. 66:486-501.

The coordinates for the LT3015 Apo Fab crystal are provided as Table 19, below.

TABLE 19
LT3015 Apo Fab crystal x-ray coordinates at 2.15 Å resolution

| REMARK | 3 | REFINEMENT. | |
|---|---|---|---|
| REMARK | 3 | PROGRAM: PHENIX (phenix.refine: 1.5_2) | |
| REMARK | 3 | REFINEMENT TARGET: MLHL | |
| REMARK | 3 | DATA USED IN REFINEMENT. | |
| REMARK | 3 | RESOLUTION RANGE HIGH (ANGSTROMS): 2.149 | |
| REMARK | 3 | RESOLUTION RANGE LOW (ANGSTROMS): 46.152 | |
| REMARK | 3 | MIN(FOBS/SIGMA_FOBS): 0.20 | |
| REMARK | 3 | COMPLETENESS FOR RANGE (%): 89.42 | |
| REMARK | 3 | NUMBER OF REFLECTIONS: 26305 | |
| REMARK | 3 | FIT TO DATA USED IN REFINEMENT. | |
| REMARK | 3 | R VALUE (WORKING + TEST SET): 0.2404 | |
| REMARK | 3 | R VALUE (WORKING SET): 0.2379 | |
| REMARK | 3 | FREE R VALUE: 0.2871 | |
| REMARK | 3 | FREE R VALUE TEST SET SIZE (%): 5.11 | |
| REMARK | 3 | FREE R VALUE TEST SET COUNT: 1345 | |
| REMARK | 3 | FIT TO DATA USED IN REFINEMENT (IN BINS). | |
| REMARK | 3 | BIN RESOLUTION RANGE COMPL. NWORK NFREE RWORK RFREE | |
| REMARK | 3 | 1 | 46.1629-4.6292 1.00 3031 126 0.2140 0.2549 |
| REMARK | 3 | 2 | 4.6292-3.6748 0.98 2786 161 0.1965 0.2293 |
| REMARK | 3 | 3 | 3.6748-3.2104 0.97 2695 176 0.2375 0.2924 |
| REMARK | 3 | 4 | 3.2104-2.9170 0.94 2616 136 0.2576 0.3099 |
| REMARK | 3 | 5 | 2.9170-2.7079 0.89 2479 133 0.2794 0.3660 |
| REMARK | 3 | 6 | 2.7079-2.5483 0.88 2423 125 0.2874 0.3108 |
| REMARK | 3 | 7 | 2.5483-2.4207 0.83 2310 107 0.2846 0.3484 |
| REMARK | 3 | 8 | 2.4207-2.3153 0.82 2242 123 0.2735 0.3410 |
| REMARK | 3 | 9 | 2.3153-2.2262 0.81 2196 137 0.2895 0.3377 |
| REMARK | 3 | 10 | 2.2262-2.1493 0.80 2182 121 0.2902 0.3712 |

| | | |
|---|---|---|
| REMARK | 3 | BULK SOLVENT MODELLING. |
| REMARK | 3 | METHOD USED: FLAT BULK SOLVENT MODEL |
| REMARK | 3 | SOLVENT RADIUS: 1.11 |
| REMARK | 3 | SHRINKAGE RADIUS: 0.90 |
| REMARK | 3 | GRID STEP FACTOR: 4.00 |
| REMARK | 3 | K_SOL: 0.340 |
| REMARK | 3 | B_SOL: 32.504 |
| REMARK | 3 | ERROR ESTIMATES. |
| REMARK | 3 | COORDINATE ERROR (MAXIMUM-LIKELIHOOD BASED): 0.29 |
| REMARK | 3 | PHASE ERROR (DEGREES, MAXIMUM-LIKELIHOOD BASED): 30.22 |
| REMARK | 3 | OVERALL SCALE FACTORS. |
| REMARK | 3 | SCALE = SUM(\|F_OBS\|*\|F_MODEL\|)/SUM(\|F_MODEL\|**2): 0.0312 |
| REMARK | 3 | ANISOTROPIC SCALE MATRIX ELEMENTS (IN CARTESIAN BASIS). |
| REMARK | 3 | B11: 6.1670 |
| REMARK | 3 | B22: 6.1670 |
| REMARK | 3 | B33: −8.8208 |
| REMARK | 3 | B12: 0.0000 |
| REMARK | 3 | B13: −0.0000 |
| REMARK | 3 | B23: 0.0000 |
| REMARK | 3 | R FACTOR FORMULA. |
| REMARK | 3 | R = SUM(\|\|F_OBS\|−SCALE*\|F_MODEL\|\|)/SUM(\|F_OBS\|) |
| REMARK | 3 | TOTAL MODEL STRUCTURE FACTOR (F_MODEL). |
| REMARK | 3 | F_MODEL = FB_CART * (F_CALC_ATOMS + F_BULK) |
| REMARK | 3 | F_BULK = K_SOL * EXP(−B_SOL * S**2/4) * F_MASK |
| REMARK | 3 | F_CALC_ATOMS = ATOMIC MODEL STRUCTURE FACTORS |
| REMARK | 3 | FB_CART = EXP(−H(t) * A(−1) * B * A(−1t) * H) |
| REMARK | 3 | A = orthogonalization matrix, H = MILLER INDEX |
| REMARK | 3 | (t) = TRANSPOSE, (−1) = INVERSE |
| REMARK | 3 | STRUCTURE FACTORS CALCULATION ALGORITHM: FFT |
| REMARK | 3 | DEVIATIONS FROM IDEAL VALUES. |
| REMARK | 3 | RMSD MAX COUNT |
| REMARK | 3 | BOND: 0.014 0.386 3448 |
| REMARK | 3 | ANGLE: 1.479 27.809 4679 |
| REMARK | 3 | CHIRALITY: 0.076 0.282 516 |
| REMARK | 3 | PLANARITY: 0.008 0.125 597 |
| REMARK | 3 | DIHEDRAL: 19.167 84.732 1207 |
| REMARK | 3 | MIN NONBONDED DISTANCE: 2.063 |
| REMARK | 3 | ATOMIC DISPLACEMENT PARAMETERS. |
| REMARK | 3 | WILSON B: 37.57 |
| REMARK | 3 | RMS(B_ISO_OR_EQUIVALENT_BONDED): 1.05 |
| REMARK | 3 | ATOMS NUMBER OF ATOMS |
| REMARK | 3 | ISO. ANISO. |
| REMARK | 3 | ALL: 3523 0 |
| REMARK | 3 | ALL (NO H): 3523 0 |
| REMARK | 3 | SOLVENT: 160 0 |
| REMARK | 3 | NON-SOLVENT: 3363 0 |
| REMARK | 3 | HYDROGENS: 0 0 |

| CRYST1 | 84.523 84.523 145.276 90.00 90.00 90.00 P 43 21 2 | | | |
|---|---|---|---|---|
| SCALE1 | 0.011831 | 0.000000 | 0.000000 | 0.00000 |
| SCALE2 | 0.000000 | 0.011831 | 0.000000 | 0.00000 |
| SCALE3 | 0.000000 | 0.000000 | 0.006883 | 0.00000 |

ATOMS and Details

| | | | | | | | | | | | |
|---|---|---|---|---|---|---|---|---|---|---|---|
| ATOM | 1 | N | GLU | H | 1 | 25.834 | −24.065 | 34.384 | 1.00 | 61.92 | N |
| ATOM | 2 | CA | GLU | H | 1 | 25.183 | −24.039 | 33.036 | 1.00 | 61.77 | C |
| ATOM | 3 | CB | GLU | H | 1 | 24.190 | −25.207 | 32.891 | 1.00 | 62.27 | C |
| ATOM | 4 | CG | GLU | H | 1 | 24.880 | −26.530 | 32.567 | 1.00 | 63.94 | C |
| ATOM | 5 | CD | GLU | H | 1 | 26.127 | −26.325 | 31.702 | 1.00 | 65.98 | C |
| ATOM | 6 | OE1 | GLU | H | 1 | 25.993 | −25.834 | 30.549 | 1.00 | 66.25 | O |
| ATOM | 7 | OE2 | GLU | H | 1 | 27.243 | −26.637 | 32.188 | 1.00 | 66.39 | O |
| ATOM | 8 | C | GLU | H | 1 | 24.509 | −22.702 | 32.710 | 1.00 | 60.89 | C |
| ATOM | 9 | O | GLU | H | 1 | 23.922 | −22.075 | 33.610 | 1.00 | 61.28 | O |
| ATOM | 10 | N | VAL | H | 2 | 24.607 | −22.276 | 31.437 | 1.00 | 59.20 | N |
| ATOM | 11 | CA | VAL | H | 2 | 23.986 | −21.017 | 30.965 | 1.00 | 57.28 | C |
| ATOM | 12 | CB | VAL | H | 2 | 24.731 | −20.368 | 29.772 | 1.00 | 57.58 | C |
| ATOM | 13 | CG1 | VAL | H | 2 | 23.959 | −19.142 | 29.265 | 1.00 | 56.44 | C |
| ATOM | 14 | CG2 | VAL | H | 2 | 26.187 | −20.003 | 30.141 | 1.00 | 57.49 | C |
| ATOM | 15 | C | VAL | H | 2 | 22.518 | −21.234 | 30.591 | 1.00 | 56.04 | C |
| ATOM | 16 | O | VAL | H | 2 | 22.197 | −21.992 | 29.675 | 1.00 | 56.51 | O |
| ATOM | 17 | N | GLN | H | 3 | 21.997 | −20.099 | 30.668 | 1.00 | 53.86 | N |
| ATOM | 18 | CA | GLN | H | 3 | 20.670 | −20.616 | 30.901 | 1.00 | 50.76 | C |
| ATOM | 19 | CB | GLN | H | 3 | 20.604 | −21.210 | 32.322 | 1.00 | 51.85 | C |
| ATOM | 20 | CG | GLN | H | 3 | 19.287 | −21.943 | 32.691 | 1.00 | 53.06 | C |
| ATOM | 21 | CD | GLN | H | 3 | 19.355 | −22.654 | 34.051 | 1.00 | 56.58 | C |
| ATOM | 22 | OE1 | GLN | H | 3 | 20.406 | −22.688 | 34.692 | 1.00 | 58.00 | O |
| ATOM | 23 | NE2 | GLN | H | 3 | 18.236 | −23.246 | 34.481 | 1.00 | 56.99 | N |
| ATOM | 24 | C | GLN | H | 3 | 19.699 | −19.450 | 30.742 | 1.00 | 47.99 | C |
| ATOM | 25 | O | GLN | H | 3 | 20.113 | −18.292 | 30.705 | 1.00 | 47.90 | O |
| ATOM | 26 | N | LEU | H | 4 | 18.313 | −19.534 | 30.360 | 1.00 | 43.66 | N |
| ATOM | 27 | CA | LEU | H | 4 | 17.603 | −18.331 | 29.958 | 1.00 | 40.10 | C |
| ATOM | 28 | CB | LEU | H | 4 | 17.033 | −18.472 | 28.543 | 1.00 | 39.63 | C |
| ATOM | 29 | CG | LEU | H | 4 | 18.003 | −19.023 | 27.495 | 1.00 | 39.38 | C |
| ATOM | 30 | CD1 | LEU | H | 4 | 17.286 | −19.366 | 26.201 | 1.00 | 38.39 | C |
| ATOM | 31 | CD2 | LEU | H | 4 | 19.145 | −18.050 | 27.252 | 1.00 | 39.07 | C |
| ATOM | 32 | C | LEU | H | 4 | 16.505 | −18.032 | 30.959 | 1.00 | 37.92 | C |
| ATOM | 33 | O | LEU | H | 4 | 15.861 | −18.944 | 31.469 | 1.00 | 36.95 | O |
| ATOM | 34 | N | VAL | H | 5 | 16.301 | −16.754 | 31.254 | 1.00 | 35.86 | N |
| ATOM | 35 | CA | VAL | H | 5 | 15.270 | −16.387 | 32.208 | 1.00 | 34.95 | C |
| ATOM | 36 | CB | VAL | H | 5 | 15.847 | −15.672 | 33.463 | 1.00 | 34.99 | C |
| ATOM | 37 | CG1 | VAL | H | 5 | 14.710 | −15.275 | 34.390 | 1.00 | 35.39 | C |
| ATOM | 38 | CG2 | VAL | H | 5 | 16.863 | −16.570 | 34.175 | 1.00 | 34.60 | C |
| ATOM | 39 | C | VAL | H | 5 | 14.233 | −15.510 | 31.551 | 1.00 | 33.83 | C |
| ATOM | 40 | O | VAL | H | 5 | 14.577 | −14.454 | 31.041 | 1.00 | 33.82 | O |
| ATOM | 41 | N | GLN | H | 6 | 12.965 | −15.931 | 31.602 | 1.00 | 32.65 | N |
| ATOM | 42 | CA | GLN | H | 6 | 11.876 | −15.176 | 30.974 | 1.00 | 32.53 | C |
| ATOM | 43 | CB | GLN | H | 6 | 10.953 | −16.126 | 30.175 | 1.00 | 32.27 | C |
| ATOM | 44 | CG | GLN | H | 6 | 11.690 | −17.051 | 29.201 | 1.00 | 31.10 | C |
| ATOM | 45 | CD | GLN | H | 6 | 10.764 | −17.922 | 28.334 | 1.00 | 30.60 | C |
| ATOM | 46 | OE1 | GLN | H | 6 | 11.004 | −19.105 | 28.177 | 1.00 | 28.58 | O |
| ATOM | 47 | NE2 | GLN | H | 6 | 9.720 | −17.324 | 27.772 | 1.00 | 30.13 | N |
| ATOM | 48 | C | GLN | H | 6 | 11.038 | −14.385 | 31.977 | 1.00 | 32.77 | C |
| ATOM | 49 | O | GLN | H | 6 | 10.984 | −14.721 | 33.168 | 1.00 | 33.19 | O |
| ATOM | 50 | N | SER | H | 7 | 10.364 | −13.361 | 31.474 | 1.00 | 32.60 | N |
| ATOM | 51 | CA | SER | H | 7 | 9.369 | −12.603 | 32.214 | 1.00 | 33.19 | C |
| ATOM | 52 | CB | SER | H | 7 | 8.984 | −11.357 | 31.402 | 1.00 | 33.10 | C |
| ATOM | 53 | OG | SER | H | 7 | 8.535 | −11.694 | 30.092 | 1.00 | 32.95 | O |
| ATOM | 54 | C | SER | H | 7 | 8.122 | −13.409 | 32.657 | 1.00 | 33.48 | C |
| ATOM | 55 | O | SER | H | 7 | 7.923 | −14.552 | 32.248 | 1.00 | 33.22 | O |
| ATOM | 56 | N | GLY | H | 8 | 7.296 | −12.809 | 33.514 | 1.00 | 34.80 | N |
| ATOM | 57 | CA | GLY | H | 8 | 6.220 | −13.531 | 34.214 | 1.00 | 35.23 | C |
| ATOM | 58 | C | GLY | H | 8 | 4.920 | −13.664 | 33.426 | 1.00 | 35.74 | C |
| ATOM | 59 | O | GLY | H | 8 | 4.736 | −13.009 | 32.397 | 1.00 | 35.79 | O |
| ATOM | 60 | N | ALA | H | 9 | 4.023 | −14.516 | 33.918 | 1.00 | 35.94 | N |
| ATOM | 61 | CA | ALA | H | 9 | 2.750 | −14.831 | 33.247 | 1.00 | 36.25 | C |
| ATOM | 62 | CB | ALA | H | 9 | 1.814 | −15.619 | 34.176 | 1.00 | 36.25 | C |
| ATOM | 63 | C | ALA | H | 9 | 2.045 | −13.598 | 32.733 | 1.00 | 36.39 | C |
| ATOM | 64 | O | ALA | H | 9 | 2.117 | −12.529 | 33.341 | 1.00 | 35.67 | O |
| ATOM | 65 | N | GLU | H | 10 | 1.371 | −13.759 | 31.597 | 1.00 | 36.69 | N |
| ATOM | 66 | CA | GLU | H | 10 | 0.587 | −12.687 | 30.990 | 1.00 | 37.21 | C |
| ATOM | 67 | CB | GLU | H | 10 | 1.126 | −12.359 | 29.596 | 1.00 | 37.62 | C |
| ATOM | 68 | CG | GLU | H | 10 | 2.572 | −11.881 | 29.601 | 1.00 | 39.46 | C |
| ATOM | 69 | CD | GLU | H | 10 | 2.725 | −10.380 | 29.863 | 1.00 | 41.52 | C |
| ATOM | 70 | OE1 | GLU | H | 10 | 1.711 | −9.674 | 30.028 | 1.00 | 43.12 | O |
| ATOM | 71 | OE2 | GLU | H | 10 | 3.878 | −9.906 | 29.900 | 1.00 | 44.02 | O |
| ATOM | 72 | C | GLU | H | 10 | −0.873 | −13.091 | 30.907 | 1.00 | 37.22 | C |
| ATOM | 73 | O | GLU | H | 10 | −1.200 | −14.251 | 30.608 | 1.00 | 37.00 | O |
| ATOM | 74 | N | VAL | H | 11 | −1.750 | −12.146 | 31.218 | 1.00 | 37.66 | N |
| ATOM | 75 | CA | VAL | H | 11 | −3.180 | −12.368 | 31.066 | 1.00 | 38.17 | C |
| ATOM | 76 | CB | VAL | H | 11 | −3.920 | −12.515 | 32.414 | 1.00 | 38.42 | C |
| ATOM | 77 | CG1 | VAL | H | 11 | −5.369 | −12.960 | 32.175 | 1.00 | 38.00 | C |
| ATOM | 78 | CG2 | VAL | H | 11 | −3.229 | −13.519 | 33.322 | 1.00 | 37.72 | C |
| ATOM | 79 | C | VAL | H | 11 | −3.728 | −11.206 | 30.237 | 1.00 | 38.87 | C |
| ATOM | 80 | O | VAL | H | 11 | −3.653 | −10.046 | 30.632 | 1.00 | 38.21 | O |
| ATOM | 81 | N | LYS | H | 12 | −4.239 | −11.525 | 29.058 | 1.00 | 39.70 | N |
| ATOM | 82 | CA | LYS | H | 12 | −4.626 | −10.494 | 28.108 | 1.00 | 40.77 | C |
| ATOM | 83 | CB | LYS | H | 12 | −3.579 | −10.398 | 26.985 | 1.00 | 41.04 | C |
| ATOM | 84 | CG | LYS | H | 12 | −2.253 | −9.724 | 27.383 | 1.00 | 43.12 | C |
| ATOM | 85 | CD | LYS | H | 12 | −2.471 | −8.291 | 27.911 | 1.00 | 43.23 | C |
| ATOM | 86 | CE | LYS | H | 12 | −1.176 | −7.481 | 27.963 | 1.00 | 47.00 | C |
| ATOM | 87 | NZ | LYS | H | 12 | −0.055 | −8.240 | 28.584 | 1.00 | 48.69 | N |
| ATOM | 88 | C | LYS | H | 12 | −6.008 | −10.751 | 27.514 | 1.00 | 40.92 | C |
| ATOM | 89 | O | LYS | H | 12 | −6.472 | −11.891 | 27.445 | 1.00 | 40.90 | O |
| ATOM | 90 | N | LYS | H | 13 | −6.648 | −9.677 | 27.069 | 1.00 | 41.29 | N |
| ATOM | 91 | CA | LYS | H | 13 | −7.933 | −9.763 | 26.391 | 1.00 | 41.41 | C |
| ATOM | 92 | CB | LYS | H | 13 | −8.764 | −8.522 | 26.728 | 1.00 | 41.76 | C |
| ATOM | 93 | CG | LYS | H | 13 | −9.372 | −8.566 | 28.135 | 1.00 | 42.67 | C |
| ATOM | 94 | CD | LYS | H | 13 | −9.758 | −7.182 | 28.624 | 1.00 | 45.02 | C |
| ATOM | 95 | CE | LYS | H | 13 | −10.605 | −7.252 | 29.895 | 1.00 | 45.77 | C |
| ATOM | 96 | NZ | LYS | H | 13 | −10.217 | −8.401 | 30.760 | 1.00 | 44.76 | N |
| ATOM | 97 | C | LYS | H | 13 | −7.678 | −9.867 | 24.886 | 1.00 | 41.27 | C |
| ATOM | 98 | O | LYS | H | 13 | −6.676 | −9.350 | 24.410 | 1.00 | 41.47 | O |
| ATOM | 99 | N | PRO | H | 14 | −8.564 | −10.554 | 24.138 | 1.00 | 41.31 | N |
| ATOM | 100 | CA | PRO | H | 14 | −8.416 | −10.601 | 22.678 | 1.00 | 42.16 | C |
| ATOM | 101 | CB | PRO | H | 14 | −9.688 | −11.322 | 22.217 | 1.00 | 41.67 | C |
| ATOM | 102 | CG | PRO | H | 14 | −10.108 | −12.125 | 23.395 | 1.00 | 41.15 | C |
| ATOM | 103 | CD | PRO | H | 14 | −9.758 | −11.295 | 24.583 | 1.00 | 41.20 | C |
| ATOM | 104 | C | PRO | H | 14 | −8.383 | −9.189 | 22.117 | 1.00 | 42.79 | C |
| ATOM | 105 | O | PRO | H | 14 | −9.195 | −8.358 | 22.526 | 1.00 | 43.16 | O |
| ATOM | 106 | N | GLY | H | 15 | −7.441 | −8.920 | 21.216 | 1.00 | 43.19 | N |
| ATOM | 107 | CA | GLY | H | 15 | −7.253 | −7.577 | 20.682 | 1.00 | 43.96 | C |
| ATOM | 108 | C | GLY | H | 15 | −6.078 | −6.822 | 21.278 | 1.00 | 44.60 | C |
| ATOM | 109 | O | GLY | H | 15 | −5.566 | −5.894 | 20.668 | 1.00 | 44.42 | O |
| ATOM | 110 | N | GLU | H | 16 | −5.639 | −7.220 | 22.469 | 1.00 | 45.23 | N |
| ATOM | 111 | CA | GLU | H | 16 | −4.521 | −6.544 | 23.130 | 1.00 | 45.47 | C |
| ATOM | 112 | CB | GLU | H | 16 | −4.605 | −6.714 | 24.650 | 1.00 | 45.63 | C |
| ATOM | 113 | CG | GLU | H | 16 | −5.620 | −5.779 | 25.304 | 1.00 | 46.57 | C |
| ATOM | 114 | CD | GLU | H | 16 | −5.817 | −6.050 | 26.786 | 1.00 | 46.37 | C |
| ATOM | 115 | OE1 | GLU | H | 16 | −6.643 | −5.365 | 27.415 | 1.00 | 47.93 | O |
| ATOM | 116 | OE2 | GLU | H | 16 | −5.155 | −6.942 | 27.332 | 1.00 | 46.84 | O |
| ATOM | 117 | C | GLU | H | 16 | −3.166 | −7.012 | 22.605 | 1.00 | 45.41 | C |
| ATOM | 118 | O | GLU | H | 16 | −3.078 | −7.977 | 21.836 | 1.00 | 45.45 | O |
| ATOM | 119 | N | SER | H | 17 | −2.118 | −6.305 | 23.006 | 1.00 | 45.30 | N |
| ATOM | 120 | CA | SER | H | 17 | −0.763 | −6.654 | 22.615 | 1.00 | 46.05 | C |
| ATOM | 121 | CB | SER | H | 17 | −0.032 | −5.441 | 22.014 | 1.00 | 45.80 | C |
| ATOM | 122 | OG | SER | H | 17 | 0.022 | −4.372 | 22.946 | 1.00 | 47.78 | O |
| ATOM | 123 | C | SER | H | 17 | 0.029 | −7.271 | 23.781 | 1.00 | 45.91 | C |
| ATOM | 124 | O | SER | H | 17 | −0.444 | −7.347 | 24.915 | 1.00 | 46.32 | O |
| ATOM | 125 | N | LEU | H | 18 | 0.897 | −7.599 | 23.612 | 1.00 | 45.78 | N |
| ATOM | 126 | CA | LEU | H | 18 | 1.642 | −8.415 | 24.545 | 1.00 | 46.05 | C |
| ATOM | 127 | CB | LEU | H | 18 | 1.381 | −9.882 | 24.195 | 1.00 | 46.47 | C |
| ATOM | 128 | CG | LEU | H | 18 | 2.063 | −11.009 | 24.936 | 1.00 | 47.05 | C |
| ATOM | 129 | CD1 | LEU | H | 18 | 1.634 | −10.971 | 26.387 | 1.00 | 46.58 | C |
| ATOM | 130 | CD2 | LEU | H | 18 | 1.649 | −12.304 | 24.288 | 1.00 | 47.60 | C |
| ATOM | 131 | C | LEU | H | 18 | 3.110 | −8.107 | 24.358 | 1.00 | 45.42 | C |
| ATOM | 132 | O | LEU | H | 18 | 3.532 | −7.767 | 23.264 | 1.00 | 45.08 | O |
| ATOM | 133 | N | LYS | H | 19 | 4.248 | −8.280 | 25.305 | 1.00 | 45.61 | N |
| ATOM | 134 | CA | LYS | H | 19 | 5.689 | −8.098 | 25.205 | 1.00 | 45.50 | C |
| ATOM | 135 | CB | LYS | H | 19 | 6.052 | −6.612 | 25.331 | 1.00 | 46.09 | C |
| ATOM | 136 | CG | LYS | H | 19 | 7.423 | −6.234 | 24.774 | 1.00 | 47.86 | C |
| ATOM | 137 | CD | LYS | H | 19 | 7.521 | −4.707 | 24.586 | 1.00 | 51.28 | C |
| ATOM | 138 | CE | LYS | H | 19 | 8.924 | −4.294 | 24.150 | 1.00 | 54.77 | C |
| ATOM | 139 | NZ | LYS | H | 19 | 9.208 | −2.841 | 24.400 | 1.00 | 55.92 | N |
| ATOM | 140 | C | LYS | H | 19 | 6.365 | −8.922 | 26.283 | 1.00 | 44.91 | C |
| ATOM | 141 | O | LYS | H | 19 | 6.285 | −8.602 | 27.467 | 1.00 | 44.93 | O |
| ATOM | 142 | N | ILE | H | 20 | 7.033 | −9.989 | 25.868 | 1.00 | 43.80 | N |
| ATOM | 143 | CA | ILE | H | 20 | 7.685 | −10.882 | 26.819 | 1.00 | 42.84 | C |
| ATOM | 144 | CB | ILE | H | 20 | 6.984 | −12.273 | 26.892 | 1.00 | 43.41 | C |
| ATOM | 145 | CG1 | ILE | H | 20 | 6.865 | −12.937 | 25.525 | 1.00 | 43.23 | C |
| ATOM | 146 | CD1 | ILE | H | 20 | 6.118 | −14.245 | 25.575 | 1.00 | 42.43 | C |
| ATOM | 147 | CG2 | ILE | H | 20 | 5.556 | −12.099 | 27.447 | 1.00 | 44.30 | C |
| ATOM | 148 | C | ILE | H | 20 | 9.189 | −10.953 | 26.566 | 1.00 | 41.66 | C |
| ATOM | 149 | O | ILE | H | 20 | 9.642 | −10.701 | 25.452 | 1.00 | 41.56 | O |
| ATOM | 150 | N | SER | H | 21 | 9.956 | −11.239 | 27.610 | 1.00 | 39.59 | N |
| ATOM | 151 | CA | SER | H | 21 | 11.393 | −11.101 | 27.540 | 1.00 | 38.58 | C |
| ATOM | 152 | CB | SER | H | 21 | 11.846 | −9.950 | 28.446 | 1.00 | 38.87 | C |
| ATOM | 153 | OG | SER | H | 21 | 11.780 | −10.348 | 29.809 | 1.00 | 39.45 | O |
| ATOM | 154 | C | SER | H | 21 | 12.105 | −12.380 | 27.927 | 1.00 | 37.68 | C |
| ATOM | 155 | O | SER | H | 21 | 11.553 | −13.229 | 28.621 | 1.00 | 36.95 | O |
| ATOM | 156 | N | CYS | H | 22 | 13.351 | −12.490 | 27.484 | 1.00 | 36.98 | N |
| ATOM | 157 | CA | CYS | H | 22 | 14.153 | −13.693 | 27.629 | 1.00 | 36.92 | C |
| ATOM | 158 | CB | CYS | H | 22 | 14.011 | −14.555 | 26.360 | 1.00 | 36.13 | C |
| ATOM | 159 | SG | CYS | H | 22 | 15.111 | −15.958 | 26.217 | 1.00 | 36.02 | S |
| ATOM | 160 | C | CYS | H | 22 | 15.587 | −13.215 | 27.792 | 1.00 | 37.61 | C |
| ATOM | 161 | O | CYS | H | 22 | 16.214 | −12.797 | 26.810 | 1.00 | 36.86 | O |
| ATOM | 162 | N | GLN | H | 23 | 16.087 | −13.237 | 29.033 | 1.00 | 38.13 | N |
| ATOM | 163 | CA | GLN | H | 23 | 17.420 | −12.711 | 29.336 | 1.00 | 38.96 | C |
| ATOM | 164 | CB | GLN | H | 23 | 17.406 | −11.876 | 30.636 | 1.00 | 39.21 | C |
| ATOM | 165 | CG | GLN | H | 23 | 18.667 | −11.013 | 30.862 | 1.00 | 38.86 | C |
| ATOM | 166 | CD | GLN | H | 23 | 18.399 | −9.839 | 31.821 | 1.00 | 39.77 | C |
| ATOM | 167 | OE1 | GLN | H | 23 | 17.618 | −9.959 | 32.768 | 1.00 | 39.36 | O |
| ATOM | 168 | NE2 | GLN | H | 23 | 19.045 | −8.708 | 31.572 | 1.00 | 38.73 | N |
| ATOM | 169 | C | GLN | H | 23 | 18.396 | −13.842 | 29.478 | 1.00 | 39.32 | C |
| ATOM | 170 | O | GLN | H | 23 | 18.138 | −14.782 | 30.222 | 1.00 | 39.73 | O |
| ATOM | 171 | N | ALA | H | 24 | 19.502 | −13.789 | 28.779 | 1.00 | 39.99 | N |
| ATOM | 172 | CA | ALA | H | 24 | 20.548 | −14.751 | 29.041 | 1.00 | 41.53 | C |
| ATOM | 173 | CB | ALA | H | 24 | 21.387 | −14.946 | 27.837 | 1.00 | 41.35 | C |
| ATOM | 174 | C | ALA | H | 24 | 21.394 | −14.355 | 30.247 | 1.00 | 42.85 | C |
| ATOM | 175 | O | ALA | H | 24 | 21.865 | −13.255 | 30.337 | 1.00 | 42.83 | O |
| ATOM | 176 | N | PHE | H | 25 | 21.562 | −15.289 | 31.163 | 1.00 | 44.84 | N |
| ATOM | 177 | CA | PHE | H | 25 | 22.256 | −15.065 | 32.426 | 1.00 | 46.88 | C |
| ATOM | 178 | CB | PHE | H | 25 | 21.519 | −15.696 | 33.613 | 1.00 | 46.58 | C |
| ATOM | 179 | CG | PHE | H | 25 | 20.494 | −14.799 | 34.274 | 1.00 | 45.88 | C |
| ATOM | 180 | CD1 | PHE | H | 25 | 20.356 | −14.789 | 35.624 | 1.00 | 45.38 | C |
| ATOM | 181 | CE1 | PHE | H | 25 | 19.427 | −14.010 | 36.228 | 1.00 | 46.27 | C |
| ATOM | 182 | CZ | PHE | H | 25 | 18.607 | −13.232 | 35.501 | 1.00 | 45.67 | C |
| ATOM | 183 | CE2 | PHE | H | 25 | 18.719 | −13.219 | 34.157 | 1.00 | 46.87 | C |
| ATOM | 184 | CD2 | PHE | H | 25 | 19.651 | −14.007 | 33.546 | 1.00 | 46.37 | C |
| ATOM | 185 | C | PHE | H | 25 | 23.649 | −15.661 | 32.288 | 1.00 | 48.43 | C |
| ATOM | 186 | O | PHE | H | 25 | 24.283 | −15.992 | 33.258 | 1.00 | 49.07 | O |
| ATOM | 187 | N | GLY | H | 26 | 24.079 | −15.838 | 31.051 | 1.00 | 49.84 | N |
| ATOM | 188 | CA | GLY | H | 26 | 25.255 | −16.598 | 30.677 | 1.00 | 51.65 | C |
| ATOM | 189 | C | GLY | H | 26 | 26.030 | −15.742 | 29.717 | 1.00 | 52.72 | C |
| ATOM | 190 | O | GLY | H | 26 | 25.587 | −14.675 | 29.441 | 1.00 | 53.38 | O |
| ATOM | 191 | N | TYR | H | 27 | 27.233 | −16.130 | 29.334 | 1.00 | 53.74 | N |
| ATOM | 192 | CA | TYR | H | 27 | 27.596 | −16.421 | 27.936 | 1.00 | 54.57 | C |
| ATOM | 193 | CB | TYR | H | 27 | 26.706 | −17.500 | 27.314 | 1.00 | 54.85 | C |
| ATOM | 194 | CG | TYR | H | 27 | 27.338 | −18.300 | 26.195 | 1.00 | 57.00 | C |
| ATOM | 195 | CD1 | TYR | H | 27 | 27.609 | −19.626 | 26.342 | 1.00 | 58.28 | C |
| ATOM | 196 | CE1 | TYR | H | 27 | 28.169 | −20.349 | 25.336 | 1.00 | 59.96 | C |
| ATOM | 197 | CZ | TYR | H | 27 | 28.471 | −19.762 | 24.163 | 1.00 | 59.37 | C |
| ATOM | 198 | OH | TYR | H | 27 | 29.040 | −20.509 | 23.168 | 1.00 | 59.41 | O |
| ATOM | 199 | CE2 | TYR | H | 27 | 28.224 | −18.452 | 23.983 | 1.00 | 58.71 | C |
| ATOM | 200 | CD2 | TYR | H | 27 | 27.655 | −17.726 | 24.991 | 1.00 | 58.60 | C |
| ATOM | 201 | C | TYR | H | 27 | 27.641 | −15.184 | 27.046 | 1.00 | 54.42 | C |
| ATOM | 202 | O | TYR | H | 27 | 27.716 | −14.082 | 27.515 | 1.00 | 54.62 | O |
| ATOM | 203 | N | GLY | H | 28 | 27.599 | −15.388 | 25.741 | 1.00 | 54.14 | N |
| ATOM | 204 | CA | GLY | H | 28 | 27.436 | −14.288 | 24.825 | 1.00 | 53.16 | C |
| ATOM | 205 | C | GLY | H | 28 | 26.249 | −14.345 | 23.889 | 1.00 | 52.39 | C |
| ATOM | 206 | O | GLY | H | 28 | 26.195 | −15.055 | 22.920 | 1.00 | 52.99 | O |
| ATOM | 207 | N | PHE | H | 29 | 25.356 | −13.434 | 24.160 | 1.00 | 51.10 | N |
| ATOM | 208 | CA | PHE | H | 29 | 24.047 | −13.351 | 23.638 | 1.00 | 49.40 | C |
| ATOM | 209 | CB | PHE | H | 29 | 23.360 | −12.272 | 24.432 | 1.00 | 48.87 | C |
| ATOM | 210 | CG | PHE | H | 29 | 22.049 | −11.885 | 23.946 | 1.00 | 46.97 | C |
| ATOM | 211 | CD1 | PHE | H | 29 | 20.954 | −12.594 | 24.277 | 1.00 | 44.85 | C |
| ATOM | 212 | CE1 | PHE | H | 29 | 19.793 | −12.206 | 23.880 | 1.00 | 43.75 | C |
| ATOM | 213 | CZ | PHE | H | 29 | 19.675 | −11.097 | 23.162 | 1.00 | 44.21 | C |
| ATOM | 214 | CE2 | PHE | H | 29 | 20.736 | −10.376 | 22.832 | 1.00 | 44.59 | C |
| ATOM | 215 | CD2 | PHE | H | 29 | 21.904 | −10.758 | 23.217 | 1.00 | 44.88 | C |
| ATOM | 216 | C | PHE | H | 29 | 24.072 | −13.093 | 22.155 | 1.00 | 49.20 | C |
| ATOM | 217 | O | PHE | H | 29 | 23.232 | −13.582 | 21.473 | 1.00 | 48.66 | O |
| ATOM | 218 | N | ILE | H | 30 | 25.015 | −12.295 | 21.658 | 1.00 | 48.60 | N |
| ATOM | 219 | CA | ILE | H | 30 | 24.981 | −11.817 | 20.262 | 1.00 | 48.15 | C |
| ATOM | 220 | CB | ILE | H | 30 | 25.968 | −10.624 | 20.002 | 1.00 | 48.54 | C |
| ATOM | 221 | CG1 | ILE | H | 30 | 27.427 | −11.056 | 20.241 | 1.00 | 48.69 | C |
| ATOM | 222 | CD1 | ILE | H | 30 | 28.444 | −10.354 | 19.354 | 1.00 | 51.03 | C |
| ATOM | 223 | CG2 | ILE | H | 30 | 25.572 | −9.409 | 20.851 | 1.00 | 49.40 | C |
| ATOM | 224 | C | ILE | H | 30 | 25.198 | −12.932 | 19.227 | 1.00 | 47.17 | C |
| ATOM | 225 | O | ILE | H | 30 | 24.843 | −12.783 | 18.054 | 1.00 | 47.88 | O |
| ATOM | 226 | N | ASN | H | 31 | 25.779 | −14.040 | 19.659 | 1.00 | 46.15 | N |
| ATOM | 227 | CA | ASN | H | 31 | 26.031 | −15.167 | 18.761 | 1.00 | 45.39 | C |
| ATOM | 228 | CB | ASN | H | 31 | 27.378 | −15.816 | 19.093 | 1.00 | 45.89 | C |
| ATOM | 229 | CG | ASN | H | 31 | 28.587 | −15.054 | 18.486 | 1.00 | 48.32 | C |
| ATOM | 230 | OD1 | ASN | H | 31 | 28.594 | −13.819 | 18.395 | 1.00 | 48.79 | O |
| ATOM | 231 | ND2 | ASN | H | 31 | 29.616 | −15.809 | 18.079 | 1.00 | 50.04 | N |
| ATOM | 232 | C | ASN | H | 31 | 24.918 | −16.239 | 18.786 | 1.00 | 44.56 | C |
| ATOM | 233 | O | ASN | H | 31 | 24.981 | −17.216 | 18.036 | 1.00 | 44.17 | O |
| ATOM | 234 | N | TYR | H | 32 | 23.908 | −16.053 | 19.639 | 1.00 | 42.83 | N |
| ATOM | 235 | CA | TYR | H | 32 | 22.856 | −17.062 | 19.831 | 1.00 | 41.52 | C |
| ATOM | 236 | CB | TYR | H | 32 | 21.980 | −16.696 | 21.038 | 1.00 | 41.99 | C |
| ATOM | 237 | CG | TYR | H | 32 | 22.566 | −16.931 | 22.412 | 1.00 | 42.69 | C |
| ATOM | 238 | CD1 | TYR | H | 32 | 23.916 | −17.259 | 22.593 | 1.00 | 43.38 | C |
| ATOM | 239 | CE1 | TYR | H | 32 | 24.443 | −17.463 | 23.864 | 1.00 | 43.12 | C |
| ATOM | 240 | CZ | TYR | H | 32 | 23.622 | −17.315 | 24.972 | 1.00 | 44.43 | C |
| ATOM | 241 | OH | TYR | H | 32 | 24.116 | −17.508 | 26.237 | 1.00 | 44.92 | O |
| ATOM | 242 | CE2 | TYR | H | 32 | 22.281 | −16.978 | 24.823 | 1.00 | 44.13 | C |
| ATOM | 243 | CD2 | TYR | H | 32 | 21.764 | −16.779 | 23.547 | 1.00 | 43.75 | C |
| ATOM | 244 | C | TYR | H | 32 | 21.919 | −17.163 | 18.641 | 1.00 | 40.18 | C |
| ATOM | 245 | O | TYR | H | 32 | 21.559 | −16.155 | 18.029 | 1.00 | 39.28 | O |
| ATOM | 246 | N | LEU | H | 33 | 21.502 | −18.383 | 18.334 | 1.00 | 38.88 | N |
| ATOM | 247 | CA | LEU | H | 33 | 20.303 | −18.566 | 17.520 | 1.00 | 37.76 | C |
| ATOM | 248 | CB | LEU | H | 33 | 20.454 | −19.697 | 16.497 | 1.00 | 37.54 | C |
| ATOM | 249 | CG | LEU | H | 33 | 21.526 | −19.602 | 15.400 | 1.00 | 38.20 | C |
| ATOM | 250 | CD1 | LEU | H | 33 | 21.367 | −20.749 | 14.402 | 1.00 | 34.13 | C |
| ATOM | 251 | CD2 | LEU | H | 33 | 21.468 | −18.247 | 14.703 | 1.00 | 38.43 | C |
| ATOM | 252 | C | LEU | H | 33 | 19.153 | −18.829 | 18.486 | 1.00 | 36.62 | C |
| ATOM | 253 | O | LEU | H | 33 | 18.953 | −19.956 | 18.974 | 1.00 | 36.31 | O |
| ATOM | 254 | N | ILE | H | 34 | 18.417 | −17.774 | 18.775 | 1.00 | 35.70 | N |
| ATOM | 255 | CA | ILE | H | 34 | 17.340 | −17.830 | 19.766 | 1.00 | 35.01 | C |
| ATOM | 256 | CB | ILE | H | 34 | 17.239 | −16.508 | 20.541 | 1.00 | 35.29 | C |
| ATOM | 257 | CG1 | ILE | H | 34 | 18.479 | −16.320 | 21.434 | 1.00 | 34.28 | C |
| ATOM | 258 | CD1 | ILE | H | 34 | 18.625 | −14.891 | 21.977 | 1.00 | 32.85 | C |
| ATOM | 259 | CG2 | ILE | H | 34 | 15.932 | −16.432 | 21.364 | 1.00 | 35.43 | C |
| ATOM | 260 | C | ILE | H | 34 | 16.027 | −18.094 | 19.065 | 1.00 | 34.62 | C |
| ATOM | 261 | O | ILE | H | 34 | 15.610 | −17.329 | 18.178 | 1.00 | 34.43 | O |
| ATOM | 262 | N | GLU | H | 35 | 15.383 | −19.183 | 19.465 | 1.00 | 33.48 | N |
| ATOM | 263 | CA | GLU | H | 35 | 14.072 | −19.522 | 18.937 | 1.00 | 32.72 | C |
| ATOM | 264 | CB | GLU | H | 35 | 14.033 | −20.998 | 18.554 | 1.00 | 32.30 | C |
| ATOM | 265 | CG | GLU | H | 35 | 15.080 | −21.364 | 17.512 | 1.00 | 31.32 | C |
| ATOM | 266 | CD | GLU | H | 35 | 15.119 | −22.822 | 17.246 | 1.00 | 28.89 | C |
| ATOM | 267 | OE1 | GLU | H | 35 | 15.988 | −23.509 | 17.800 | 1.00 | 31.45 | O |
| ATOM | 268 | OE2 | GLU | H | 35 | 14.254 | −23.304 | 16.493 | 1.00 | 32.14 | O |
| ATOM | 269 | C | GLU | H | 35 | 12.980 | −19.236 | 19.946 | 1.00 | 32.14 | C |
| ATOM | 270 | O | GLU | H | 35 | 13.197 | −19.339 | 21.149 | 1.00 | 32.05 | O |
| ATOM | 271 | N | TRP | H | 36 | 11.809 | −18.878 | 19.435 | 1.00 | 31.89 | N |
| ATOM | 272 | CA | TRP | H | 36 | 10.585 | −18.811 | 20.223 | 1.00 | 31.81 | C |
| ATOM | 273 | CB | TRP | H | 36 | 9.917 | −17.455 | 20.081 | 1.00 | 31.57 | C |
| ATOM | 274 | CG | TRP | H | 36 | 10.704 | −16.358 | 20.728 | 1.00 | 34.68 | C |
| ATOM | 275 | CD1 | TRP | H | 36 | 11.698 | −15.605 | 20.156 | 1.00 | 34.70 | C |
| ATOM | 276 | NE1 | TRP | H | 36 | 12.175 | −14.691 | 21.066 | 1.00 | 36.00 | N |
| ATOM | 277 | CE2 | TRP | H | 36 | 11.516 | −14.860 | 22.257 | 1.00 | 36.22 | C |
| ATOM | 278 | CD2 | TRP | H | 36 | 10.586 | −15.908 | 22.084 | 1.00 | 34.31 | C |
| ATOM | 279 | CE3 | TRP | H | 36 | 9.754 | −16.264 | 23.159 | 1.00 | 33.12 | C |
| ATOM | 280 | CZ3 | TRP | H | 36 | 9.896 | −15.582 | 24.366 | 1.00 | 31.34 | C |
| ATOM | 281 | CH2 | TRP | H | 36 | 10.839 | −14.551 | 24.505 | 1.00 | 32.97 | C |
| ATOM | 282 | CZ2 | TRP | H | 36 | 11.659 | −14.179 | 23.471 | 1.00 | 34.38 | C |
| ATOM | 283 | C | TRP | H | 36 | 9.652 | −19.936 | 19.787 | 1.00 | 31.38 | C |
| ATOM | 284 | O | TRP | H | 36 | 9.401 | −20.126 | 18.593 | 1.00 | 31.51 | O |
| ATOM | 285 | N | ILE | H | 37 | 9.174 | −20.692 | 20.766 | 1.00 | 29.93 | N |
| ATOM | 286 | CA | ILE | H | 37 | 8.379 | −21.875 | 20.535 | 1.00 | 30.11 | C |
| ATOM | 287 | CB | ILE | H | 37 | 9.234 | −23.167 | 20.703 | 1.00 | 29.35 | C |
| ATOM | 288 | CG1 | ILE | H | 37 | 10.377 | −23.228 | 19.669 | 1.00 | 29.00 | C |
| ATOM | 289 | CD1 | ILE | H | 37 | 11.429 | −24.329 | 19.922 | 1.00 | 27.78 | C |
| ATOM | 290 | CG2 | ILE | H | 37 | 8.376 | −24.414 | 20.676 | 1.00 | 29.99 | C |
| ATOM | 291 | C | ILE | H | 37 | 7.274 | −21.814 | 21.585 | 1.00 | 31.10 | C |
| ATOM | 292 | O | ILE | H | 37 | 7.544 | −21.570 | 22.780 | 1.00 | 31.05 | O |
| ATOM | 293 | N | ARG | H | 38 | 6.031 | −21.991 | 21.153 | 1.00 | 31.48 | N |
| ATOM | 294 | CA | ARG | H | 38 | 4.928 | −22.047 | 22.102 | 1.00 | 32.51 | C |
| ATOM | 295 | CB | ARG | H | 38 | 3.887 | −20.958 | 21.837 | 1.00 | 32.14 | C |
| ATOM | 296 | CG | ARG | H | 38 | 3.117 | −21.123 | 20.531 | 1.00 | 33.47 | C |
| ATOM | 297 | CD | ARG | H | 38 | 2.180 | −19.968 | 20.288 | 1.00 | 33.62 | C |
| ATOM | 298 | NE | ARG | H | 38 | 1.144 | −20.337 | 19.324 | 1.00 | 35.34 | N |
| ATOM | 299 | CZ | ARG | H | 38 | 0.185 | −19.532 | 18.884 | 1.00 | 34.38 | C |
| ATOM | 300 | NH1 | ARG | H | 38 | 0.105 | −18.272 | 19.293 | 1.00 | 33.52 | N |
| ATOM | 301 | NH2 | ARG | H | 38 | −0.699 | −20.000 | 18.020 | 1.00 | 35.76 | N |
| ATOM | 302 | C | ARG | H | 38 | 4.309 | −23.437 | 22.164 | 1.00 | 33.45 | C |
| ATOM | 303 | O | ARG | H | 38 | 4.260 | −24.181 | 21.167 | 1.00 | 33.36 | O |
| ATOM | 304 | N | GLN | H | 39 | 3.883 | −23.800 | 23.365 | 1.00 | 34.33 | N |
| ATOM | 305 | CA | GLN | H | 39 | 3.260 | −25.070 | 23.592 | 1.00 | 35.47 | C |
| ATOM | 306 | CB | GLN | H | 39 | 3.966 | −25.845 | 24.688 | 1.00 | 35.39 | C |
| ATOM | 307 | CG | GLN | H | 39 | 3.313 | −27.176 | 24.938 | 1.00 | 35.69 | C |
| ATOM | 308 | CD | GLN | H | 39 | 4.222 | −28.116 | 25.637 | 1.00 | 37.35 | C |
| ATOM | 309 | OE1 | GLN | H | 39 | 4.878 | −27.749 | 26.623 | 1.00 | 38.48 | O |
| ATOM | 310 | NE2 | GLN | H | 39 | 4.293 | −29.346 | 25.136 | 1.00 | 34.77 | N |
| ATOM | 311 | C | GLN | H | 39 | 1.803 | −24.846 | 23.944 | 1.00 | 36.83 | C |
| ATOM | 312 | O | GLN | H | 39 | 1.474 | −24.167 | 24.917 | 1.00 | 36.41 | O |
| ATOM | 313 | N | MET | H | 40 | 0.953 | −25.442 | 23.120 | 1.00 | 38.67 | N |
| ATOM | 314 | CA | MET | H | 40 | −0.479 | −25.247 | 23.156 | 1.00 | 40.76 | C |
| ATOM | 315 | CB | MET | H | 40 | −1.065 | −25.666 | 21.798 | 1.00 | 41.23 | C |
| ATOM | 316 | CG | MET | H | 40 | −0.255 | −25.171 | 20.594 | 1.00 | 40.99 | C |
| ATOM | 317 | SD | MET | H | 40 | 0.080 | −23.385 | 20.631 | 1.00 | 42.46 | S |
| ATOM | 318 | CE | MET | H | 40 | −1.592 | −22.699 | 20.513 | 1.00 | 42.35 | C |
| ATOM | 319 | C | MET | H | 40 | −1.099 | −26.051 | 24.302 | 1.00 | 42.06 | C |
| ATOM | 320 | O | MET | H | 40 | −0.532 | −27.062 | 24.726 | 1.00 | 41.30 | O |
| ATOM | 321 | N | PRO | H | 41 | −2.328 | −25.754 | 24.705 | 1.00 | 43.93 | N |
| ATOM | 322 | CA | PRO | H | 41 | −2.774 | −26.120 | 26.060 | 1.00 | 45.24 | C |
| ATOM | 323 | CB | PRO | H | 41 | −4.210 | −25.589 | 26.103 | 1.00 | 45.18 | C |
| ATOM | 324 | CG | PRO | H | 41 | −4.214 | −24.452 | 25.143 | 1.00 | 45.09 | C |
| ATOM | 325 | CD | PRO | H | 41 | −3.274 | −24.843 | 24.038 | 1.00 | 43.83 | C |
| ATOM | 326 | C | PRO | H | 41 | −2.762 | −27.633 | 26.402 | 1.00 | 46.36 | C |
| ATOM | 327 | O | PRO | H | 41 | −2.361 | −27.961 | 27.518 | 1.00 | 47.36 | O |
| ATOM | 328 | N | GLY | H | 42 | −3.177 | −28.522 | 25.497 | 1.00 | 46.55 | N |
| ATOM | 329 | CA | GLY | H | 42 | −2.832 | −29.938 | 25.597 | 1.00 | 47.33 | C |
| ATOM | 330 | C | GLY | H | 42 | −2.095 | −30.386 | 24.344 | 1.00 | 47.50 | C |
| ATOM | 331 | O | GLY | H | 42 | −2.147 | −31.549 | 23.941 | 1.00 | 47.80 | O |
| ATOM | 332 | N | GLN | H | 43 | −1.357 | −29.472 | 23.735 | 1.00 | 47.35 | N |
| ATOM | 333 | CA | GLN | H | 43 | −0.937 | −29.734 | 22.369 | 1.00 | 46.94 | C |
| ATOM | 334 | CB | GLN | H | 43 | −1.632 | −28.773 | 21.372 | 1.00 | 47.37 | C |
| ATOM | 335 | CG | GLN | H | 43 | −3.110 | −29.053 | 21.117 | 1.00 | 51.27 | C |
| ATOM | 336 | CD | GLN | H | 43 | −3.938 | −27.774 | 20.932 | 1.00 | 55.64 | C |
| ATOM | 337 | OE1 | GLN | H | 43 | −3.700 | −26.987 | 20.004 | 1.00 | 58.85 | O |
| ATOM | 338 | NE2 | GLN | H | 43 | −4.914 | −27.568 | 21.812 | 1.00 | 55.26 | N |
| ATOM | 339 | C | GLN | H | 43 | 0.574 | −29.810 | 22.129 | 1.00 | 45.00 | C |
| ATOM | 340 | O | GLN | H | 43 | 1.399 | −30.002 | 23.035 | 1.00 | 44.90 | O |
| ATOM | 341 | N | GLY | H | 44 | 0.907 | −29.681 | 20.863 | 1.00 | 42.87 | N |
| ATOM | 342 | CA | GLY | H | 44 | 2.249 | −29.819 | 20.444 | 1.00 | 40.84 | C |
| ATOM | 343 | C | GLY | H | 44 | 2.943 | −28.500 | 20.574 | 1.00 | 38.78 | C |
| ATOM | 344 | O | GLY | H | 44 | 2.529 | −27.602 | 21.313 | 1.00 | 38.12 | O |
| ATOM | 345 | N | LEU | H | 45 | 4.019 | −28.413 | 19.825 | 1.00 | 37.30 | N |
| ATOM | 346 | CA | LEU | H | 45 | 4.854 | −27.260 | 19.834 | 1.00 | 35.84 | C |
| ATOM | 347 | CB | LEU | H | 45 | 6.310 | −27.716 | 19.947 | 1.00 | 35.06 | C |
| ATOM | 348 | CG | LEU | H | 45 | 6.569 | −28.481 | 21.259 | 1.00 | 33.94 | C |
| ATOM | 349 | CD1 | LEU | H | 45 | 7.922 | −29.167 | 21.308 | 1.00 | 32.88 | C |
| ATOM | 350 | CD2 | LEU | H | 45 | 6.370 | −27.601 | 22.490 | 1.00 | 31.78 | C |
| ATOM | 351 | C | LEU | H | 45 | 4.553 | −26.527 | 18.542 | 1.00 | 35.33 | C |
| ATOM | 352 | O | LEU | H | 45 | 4.378 | −27.154 | 17.498 | 1.00 | 35.69 | O |
| ATOM | 353 | N | GLU | H | 46 | 4.421 | −25.212 | 18.630 | 1.00 | 34.21 | N |
| ATOM | 354 | CA | GLU | H | 46 | 4.294 | −24.377 | 17.446 | 1.00 | 33.77 | C |
| ATOM | 355 | CB | GLU | H | 46 | 3.002 | −23.554 | 17.485 | 1.00 | 33.78 | C |
| ATOM | 356 | CG | GLU | H | 46 | 1.753 | −24.333 | 17.053 | 1.00 | 38.01 | C |
| ATOM | 357 | CD | GLU | H | 46 | 0.476 | −23.482 | 17.053 | 1.00 | 39.86 | C |
| ATOM | 358 | OE1 | GLU | H | 46 | −0.623 | −24.064 | 17.035 | 1.00 | 43.39 | O |
| ATOM | 359 | OE2 | GLU | H | 46 | 0.558 | −22.240 | 17.082 | 1.00 | 41.24 | O |
| ATOM | 360 | C | GLU | H | 46 | 5.517 | −23.474 | 17.408 | 1.00 | 32.82 | C |
| ATOM | 361 | O | GLU | H | 46 | 5.827 | −22.791 | 18.391 | 1.00 | 32.51 | O |
| ATOM | 362 | N | TRP | H | 47 | 6.239 | −23.514 | 16.296 | 1.00 | 31.92 | N |
| ATOM | 363 | CA | TRP | H | 47 | 7.464 | −22.763 | 16.150 | 1.00 | 31.06 | C |
| ATOM | 364 | CB | TRP | H | 47 | 8.369 | −23.440 | 15.127 | 1.00 | 31.05 | C |
| ATOM | 365 | CG | TRP | H | 47 | 9.713 | −22.823 | 14.992 | 1.00 | 31.19 | C |
| ATOM | 366 | CD1 | TRP | H | 47 | 10.784 | −23.002 | 15.827 | 1.00 | 31.51 | C |
| ATOM | 367 | NE1 | TRP | H | 47 | 11.856 | −22.276 | 15.379 | 1.00 | 31.03 | N |
| ATOM | 368 | CE2 | TRP | H | 47 | 11.503 | −21.610 | 14.235 | 1.00 | 30.40 | C |
| ATOM | 369 | CD2 | TRP | H | 47 | 10.155 | −21.927 | 13.959 | 1.00 | 30.67 | C |
| ATOM | 370 | CE3 | TRP | H | 47 | 9.540 | −21.362 | 12.828 | 1.00 | 31.78 | C |
| ATOM | 371 | CZ3 | TRP | H | 47 | 10.297 | −20.502 | 12.009 | 1.00 | 32.46 | C |
| ATOM | 372 | CH2 | TRP | H | 47 | 11.633 | −20.203 | 12.317 | 1.00 | 32.52 | C |
| ATOM | 373 | CZ2 | TRP | H | 47 | 12.256 | −20.747 | 13.425 | 1.00 | 32.55 | C |
| ATOM | 374 | C | TRP | H | 47 | 7.051 | −21.377 | 15.692 | 1.00 | 31.67 | C |
| ATOM | 375 | O | TRP | H | 47 | 6.313 | −21.238 | 14.717 | 1.00 | 31.03 | O |
| ATOM | 376 | N | ILE | H | 48 | 7.507 | −20.355 | 16.417 | 1.00 | 31.59 | N |
| ATOM | 377 | CA | ILE | H | 48 | 7.119 | −18.972 | 16.154 | 1.00 | 31.66 | C |
| ATOM | 378 | CB | ILE | H | 48 | 6.957 | −18.170 | 17.472 | 1.00 | 31.12 | C |
| ATOM | 379 | CG1 | ILE | H | 48 | 5.735 | −18.653 | 18.248 | 1.00 | 30.02 | C |
| ATOM | 380 | CD1 | ILE | H | 48 | 5.881 | −18.419 | 19.750 | 1.00 | 28.32 | C |
| ATOM | 381 | CG2 | ILE | H | 48 | 6.800 | −16.657 | 17.214 | 1.00 | 32.69 | C |
| ATOM | 382 | C | ILE | H | 48 | 8.118 | −18.277 | 15.228 | 1.00 | 32.49 | C |
| ATOM | 383 | O | ILE | H | 48 | 7.725 | −17.658 | 14.225 | 1.00 | 32.16 | O |
| ATOM | 384 | N | GLY | H | 49 | 9.402 | −18.368 | 15.575 | 1.00 | 32.96 | N |
| ATOM | 385 | CA | GLY | H | 49 | 10.450 | −17.669 | 14.833 | 1.00 | 34.01 | C |
| ATOM | 386 | C | GLY | H | 49 | 11.832 | −17.838 | 15.435 | 1.00 | 35.33 | C |
| ATOM | 387 | O | GLY | H | 49 | 12.004 | −18.539 | 16.453 | 1.00 | 35.62 | O |
| ATOM | 388 | N | LEU | H | 50 | 12.819 | −17.216 | 14.792 | 1.00 | 36.21 | N |
| ATOM | 389 | CA | LEU | H | 50 | 14.208 | −17.245 | 15.244 | 1.00 | 37.55 | C |
| ATOM | 390 | CB | LEU | H | 50 | 15.013 | −18.311 | 14.484 | 1.00 | 37.07 | C |
| ATOM | 391 | CG | LEU | H | 50 | 16.542 | −18.464 | 14.651 | 1.00 | 37.14 | C |
| ATOM | 392 | CD1 | LEU | H | 50 | 16.988 | −19.864 | 14.332 | 1.00 | 36.45 | C |
| ATOM | 393 | CD2 | LEU | H | 50 | 17.336 | −17.448 | 13.790 | 1.00 | 37.64 | C |
| ATOM | 394 | C | LEU | H | 50 | 14.829 | −15.842 | 15.099 | 1.00 | 38.86 | C |
| ATOM | 395 | O | LEU | H | 50 | 14.493 | −15.096 | 14.174 | 1.00 | 39.01 | O |
| ATOM | 396 | N | ILE | H | 51 | 15.700 | −15.485 | 16.039 | 1.00 | 40.20 | N |
| ATOM | 397 | CA | ILE | H | 51 | 16.512 | −14.277 | 15.937 | 1.00 | 41.52 | C |
| ATOM | 398 | CB | ILE | H | 51 | 16.002 | −13.144 | 16.866 | 1.00 | 42.07 | C |
| ATOM | 399 | CG1 | ILE | H | 51 | 16.678 | −11.803 | 16.530 | 1.00 | 40.68 | C |
| ATOM | 400 | CD1 | ILE | H | 51 | 15.938 | −10.590 | 17.061 | 1.00 | 40.63 | C |
| ATOM | 401 | CG2 | ILE | H | 51 | 16.231 | −13.515 | 18.350 | 1.00 | 40.63 | C |
| ATOM | 402 | C | ILE | H | 51 | 17.966 | −14.597 | 16.272 | 1.00 | 43.06 | C |
| ATOM | 403 | O | ILE | H | 51 | 18.259 | −15.401 | 17.167 | 1.00 | 43.08 | O |
| ATOM | 404 | N | ASN | H | 52 | 18.875 | −14.012 | 15.500 | 1.00 | 44.81 | N |
| ATOM | 405 | CA | ASN | H | 52 | 20.274 | −13.924 | 15.889 | 1.00 | 46.11 | C |
| ATOM | 406 | CB | ASN | H | 52 | 21.195 | −14.419 | 14.768 | 1.00 | 46.93 | C |
| ATOM | 407 | CG | ASN | H | 52 | 22.655 | −14.570 | 15.213 | 1.00 | 48.60 | C |
| ATOM | 408 | OD1 | ASN | H | 52 | 22.985 | −14.426 | 16.389 | 1.00 | 51.00 | O |
| ATOM | 409 | ND2 | ASN | H | 52 | 23.530 | −14.854 | 14.261 | 1.00 | 50.47 | N |
| ATOM | 410 | C | ASN | H | 52 | 20.477 | −12.439 | 16.181 | 1.00 | 46.77 | C |
| ATOM | 411 | O | ASN | H | 52 | 20.285 | −11.596 | 15.286 | 1.00 | 46.96 | O |
| ATOM | 412 | N | PRO | H | 52A | 20.790 | −12.099 | 17.448 | 1.00 | 47.07 | N |
| ATOM | 413 | CA | PRO | H | 52A | 20.884 | −10.699 | 17.851 | 1.00 | 47.73 | C |
| ATOM | 414 | CB | PRO | H | 52A | 21.216 | −10.793 | 19.347 | 1.00 | 47.79 | C |
| ATOM | 415 | CG | PRO | H | 52A | 20.730 | −12.128 | 19.751 | 1.00 | 47.14 | C |
| ATOM | 416 | CD | PRO | H | 52A | 21.022 | −13.000 | 18.587 | 1.00 | 47.05 | C |
| ATOM | 417 | C | PRO | H | 52A | 21.989 | −9.956 | 17.097 | 1.00 | 48.41 | C |
| ATOM | 418 | O | PRO | H | 52A | 21.768 | −8.821 | 16.663 | 1.00 | 48.73 | O |
| ATOM | 419 | N | GLY | H | 53 | 23.144 | −10.610 | 16.939 | 1.00 | 48.93 | N |
| ATOM | 420 | CA | GLY | H | 53 | 24.259 | −10.098 | 16.149 | 1.00 | 49.33 | C |
| ATOM | 421 | C | GLY | H | 53 | 23.777 | −9.383 | 14.900 | 1.00 | 49.77 | C |
| ATOM | 422 | O | GLY | H | 53 | 23.824 | −8.145 | 14.830 | 1.00 | 49.86 | O |
| ATOM | 423 | N | SER | H | 54 | 23.279 | −10.164 | 13.941 | 1.00 | 49.90 | N |
| ATOM | 424 | CA | SER | H | 54 | 22.835 | −9.661 | 12.636 | 1.00 | 50.42 | C |
| ATOM | 425 | CB | SER | H | 54 | 22.653 | −10.832 | 11.678 | 1.00 | 50.48 | C |
| ATOM | 426 | OG | SER | H | 54 | 23.883 | −11.495 | 11.484 | 1.00 | 51.91 | O |
| ATOM | 427 | C | SER | H | 54 | 21.535 | −8.871 | 12.665 | 1.00 | 50.39 | C |
| ATOM | 428 | O | SER | H | 54 | 21.313 | −7.990 | 11.834 | 1.00 | 50.68 | O |
| ATOM | 429 | N | ASP | H | 55 | 20.680 | −9.204 | 13.628 | 1.00 | 50.64 | N |
| ATOM | 430 | CA | ASP | H | 55 | 19.246 | −8.862 | 13.607 | 1.00 | 50.68 | C |
| ATOM | 431 | CB | ASP | H | 55 | 18.991 | −7.346 | 13.454 | 1.00 | 50.72 | C |
| ATOM | 432 | CG | ASP | H | 55 | 17.725 | −6.892 | 14.183 | 1.00 | 51.99 | C |
| ATOM | 433 | OD1 | ASP | H | 55 | 17.005 | −5.997 | 13.676 | 1.00 | 52.15 | O |
| ATOM | 434 | OD2 | ASP | H | 55 | 17.443 | −7.454 | 15.264 | 1.00 | 52.73 | O |
| ATOM | 435 | C | ASP | H | 55 | 18.452 | −9.695 | 12.575 | 1.00 | 50.32 | C |
| ATOM | 436 | O | ASP | H | 55 | 17.313 | −9.350 | 12.232 | 1.00 | 50.56 | O |
| ATOM | 437 | N | TYR | H | 56 | 19.043 | −10.796 | 12.094 | 1.00 | 49.80 | N |
| ATOM | 438 | CA | TYR | H | 56 | 18.293 | −11.742 | 11.255 | 1.00 | 49.53 | C |
| ATOM | 439 | CB | TYR | H | 56 | 19.138 | −12.925 | 10.771 | 1.00 | 49.46 | C |
| ATOM | 440 | CG | TYR | H | 56 | 18.350 | −13.886 | 9.886 | 1.00 | 51.36 | C |
| ATOM | 441 | CD1 | TYR | H | 56 | 18.052 | −13.562 | 8.550 | 1.00 | 53.10 | C |
| ATOM | 442 | CE1 | TYR | H | 56 | 17.325 | −14.439 | 7.727 | 1.00 | 52.67 | C |
| ATOM | 443 | CZ | TYR | H | 56 | 16.879 | −15.646 | 8.251 | 1.00 | 53.82 | C |
| ATOM | 444 | OH | TYR | H | 56 | 16.162 | −16.514 | 7.451 | 1.00 | 54.27 | O |
| ATOM | 445 | CE2 | TYR | H | 56 | 17.155 | −15.988 | 9.574 | 1.00 | 52.47 | C |
| ATOM | 446 | CD2 | TYR | H | 56 | 17.884 | −15.110 | 10.383 | 1.00 | 51.67 | C |
| ATOM | 447 | C | TYR | H | 56 | 17.069 | −12.259 | 12.002 | 1.00 | 48.93 | C |
| ATOM | 448 | O | TYR | H | 56 | 17.148 | −12.639 | 13.172 | 1.00 | 48.77 | O |
| ATOM | 449 | N | THR | H | 57 | 15.940 | −12.247 | 11.310 | 1.00 | 48.21 | N |
| ATOM | 450 | CA | THR | H | 57 | 14.715 | −12.795 | 11.845 | 1.00 | 47.86 | C |
| ATOM | 451 | CB | THR | H | 57 | 13.769 | −11.698 | 12.376 | 1.00 | 47.64 | C |
| ATOM | 452 | OG1 | THR | H | 57 | 13.554 | −10.701 | 11.371 | 1.00 | 46.65 | O |
| ATOM | 453 | CG2 | THR | H | 57 | 14.362 | −11.041 | 13.606 | 1.00 | 47.11 | C |
| ATOM | 454 | C | THR | H | 57 | 14.019 | −13.628 | 10.783 | 1.00 | 47.83 | C |
| ATOM | 455 | O | THR | H | 57 | 13.976 | −13.254 | 9.604 | 1.00 | 47.76 | O |
| ATOM | 456 | N | ASN | H | 58 | 13.514 | −14.780 | 11.208 | 1.00 | 47.13 | N |
| ATOM | 457 | CA | ASN | H | 58 | 12.652 | −15.590 | 10.379 | 1.00 | 46.49 | C |
| ATOM | 458 | CB | ASN | H | 58 | 13.343 | −16.893 | 9.990 | 1.00 | 46.28 | C |
| ATOM | 459 | CG | ASN | H | 58 | 12.552 | −17.685 | 8.968 | 1.00 | 46.90 | C |
| ATOM | 460 | OD1 | ASN | H | 58 | 11.555 | −18.344 | 9.297 | 1.00 | 45.42 | O |
| ATOM | 461 | ND2 | ASN | H | 58 | 13.000 | −17.635 | 7.712 | 1.00 | 46.82 | N |
| ATOM | 462 | C | ASN | H | 58 | 11.378 | −15.869 | 11.152 | 1.00 | 46.29 | C |
| ATOM | 463 | O | ASN | H | 58 | 11.433 | −16.223 | 12.328 | 1.00 | 46.41 | O |
| ATOM | 464 | N | TYR | H | 59 | 10.239 | −15.703 | 10.486 | 1.00 | 45.97 | N |
| ATOM | 465 | CA | TYR | H | 59 | 8.931 | −15.843 | 11.114 | 1.00 | 45.71 | C |
| ATOM | 466 | CB | TYR | H | 59 | 8.129 | −14.547 | 10.977 | 1.00 | 45.02 | C |
| ATOM | 467 | CG | TYR | H | 59 | 8.703 | −13.368 | 11.726 | 1.00 | 45.56 | C |
| ATOM | 468 | CD1 | TYR | H | 59 | 9.596 | −12.484 | 11.105 | 1.00 | 45.26 | C |
| ATOM | 469 | CE1 | TYR | H | 59 | 10.128 | −11.390 | 11.795 | 1.00 | 45.44 | C |
| ATOM | 470 | CZ | TYR | H | 59 | 9.766 | −11.167 | 13.125 | 1.00 | 46.35 | C |
| ATOM | 471 | OH | TYR | H | 59 | 10.282 | −10.087 | 13.805 | 1.00 | 45.19 | O |
| ATOM | 472 | CE2 | TYR | H | 59 | 8.876 | −12.024 | 13.762 | 1.00 | 45.50 | C |
| ATOM | 473 | CD2 | TYR | H | 59 | 8.349 | −13.121 | 13.059 | 1.00 | 45.58 | C |
| ATOM | 474 | C | TYR | H | 59 | 8.138 | −16.970 | 10.483 | 1.00 | 45.56 | C |
| ATOM | 475 | O | TYR | H | 59 | 8.125 | −17.124 | 9.266 | 1.00 | 45.24 | O |
| ATOM | 476 | N | ASN | H | 60 | 7.483 | −17.761 | 11.321 | 1.00 | 45.53 | N |
| ATOM | 477 | CA | ASN | H | 60 | 6.444 | −18.650 | 10.854 | 1.00 | 45.87 | C |
| ATOM | 478 | CB | ASN | H | 60 | 5.882 | −19.447 | 12.028 | 1.00 | 45.45 | C |
| ATOM | 479 | CG | ASN | H | 60 | 4.797 | −20.428 | 11.617 | 1.00 | 45.05 | C |
| ATOM | 480 | OD1 | ASN | H | 60 | 3.945 | −20.133 | 10.766 | 1.00 | 43.69 | O |
| ATOM | 481 | ND2 | ASN | H | 60 | 4.806 | −21.599 | 12.246 | 1.00 | 41.41 | N |
| ATOM | 482 | C | ASN | H | 60 | 5.361 | −17.767 | 10.212 | 1.00 | 46.37 | C |
| ATOM | 483 | O | ASN | H | 60 | 4.811 | −16.871 | 10.860 | 1.00 | 45.49 | O |
| ATOM | 484 | N | GLU | H | 61 | 5.081 | −18.027 | 8.935 | 1.00 | 47.35 | N |
| ATOM | 485 | CA | GLU | H | 61 | 4.156 | −17.231 | 8.132 | 1.00 | 48.29 | C |
| ATOM | 486 | CB | GLU | H | 61 | 3.924 | −17.909 | 6.778 | 1.00 | 49.09 | C |
| ATOM | 487 | CG | GLU | H | 61 | 3.040 | −17.137 | 5.804 | 1.00 | 52.95 | C |
| ATOM | 488 | CD | GLU | H | 61 | 2.101 | −18.061 | 5.020 | 1.00 | 57.12 | C |
| ATOM | 489 | OE1 | GLU | H | 61 | 2.542 | −19.173 | 4.616 | 1.00 | 57.28 | O |
| ATOM | 490 | OE2 | GLU | H | 61 | 0.917 | −17.675 | 4.832 | 1.00 | 58.56 | O |
| ATOM | 491 | C | GLU | H | 61 | 2.833 | −16.979 | 8.838 | 1.00 | 48.17 | C |
| ATOM | 492 | O | GLU | H | 61 | 2.331 | −15.853 | 8.798 | 1.00 | 48.29 | O |
| ATOM | 493 | N | ASN | H | 62 | 2.285 | −18.005 | 9.499 | 1.00 | 47.73 | N |
| ATOM | 494 | CA | ASN | H | 62 | 1.047 | −17.860 | 10.274 | 1.00 | 47.93 | C |
| ATOM | 495 | CB | ASN | H | 62 | 0.683 | −19.164 | 10.978 | 1.00 | 48.49 | C |
| ATOM | 496 | CG | ASN | H | 62 | 0.479 | −20.316 | 10.014 | 1.00 | 49.34 | C |
| ATOM | 497 | OD1 | ASN | H | 62 | 0.011 | −20.132 | 8.894 | 1.00 | 51.20 | O |
| ATOM | 498 | ND2 | ASN | H | 62 | 0.819 | −21.517 | 10.457 | 1.00 | 50.89 | N |
| ATOM | 499 | C | ASN | H | 62 | 1.066 | −16.738 | 11.315 | 1.00 | 48.01 | C |
| ATOM | 500 | O | ASN | H | 62 | 0.024 | −16.191 | 11.661 | 1.00 | 47.82 | O |
| ATOM | 501 | N | PHE | H | 63 | 2.240 | −16.380 | 11.816 | 1.00 | 47.82 | N |
| ATOM | 502 | CA | PHE | H | 63 | 2.281 | −15.386 | 12.879 | 1.00 | 47.67 | C |
| ATOM | 503 | CB | PHE | H | 63 | 3.012 | −15.936 | 14.106 | 1.00 | 47.16 | C |
| ATOM | 504 | CG | PHE | H | 63 | 2.544 | −17.301 | 14.538 | 1.00 | 44.58 | C |
| ATOM | 505 | CD1 | PHE | H | 63 | 3.453 | −18.339 | 14.675 | 1.00 | 42.83 | C |
| ATOM | 506 | CE1 | PHE | H | 63 | 3.039 | −19.598 | 15.077 | 1.00 | 42.41 | C |
| ATOM | 507 | CZ | PHE | H | 63 | 1.691 | −19.837 | 15.336 | 1.00 | 42.91 | C |
| ATOM | 508 | CE2 | PHE | H | 63 | 0.765 | −18.810 | 15.200 | 1.00 | 42.54 | C |
| ATOM | 509 | CD2 | PHE | H | 63 | 1.199 | −17.545 | 14.805 | 1.00 | 43.56 | C |
| ATOM | 510 | C | PHE | H | 63 | 2.883 | −14.058 | 12.438 | 1.00 | 48.62 | C |
| ATOM | 511 | O | PHE | H | 63 | 2.949 | −13.110 | 13.228 | 1.00 | 49.03 | O |
| ATOM | 512 | N | LYS | H | 64 | 3.302 | −13.986 | 11.175 | 1.00 | 49.13 | N |
| ATOM | 513 | CA | LYS | H | 64 | 3.934 | −12.790 | 10.634 | 1.00 | 49.73 | C |
| ATOM | 514 | CB | LYS | H | 64 | 4.430 | −13.043 | 9.206 | 1.00 | 50.25 | C |
| ATOM | 515 | CG | LYS | H | 64 | 5.296 | −11.932 | 8.610 | 1.00 | 53.01 | C |
| ATOM | 516 | CD | LYS | H | 64 | 6.388 | −11.451 | 9.574 | 1.00 | 57.24 | C |
| ATOM | 517 | CE | LYS | H | 64 | 7.008 | −10.132 | 9.117 | 1.00 | 58.34 | C |
| ATOM | 518 | NZ | LYS | H | 64 | 7.995 | −10.365 | 8.023 | 1.00 | 60.64 | N |
| ATOM | 519 | C | LYS | H | 64 | 2.970 | −11.604 | 10.692 | 1.00 | 49.37 | C |
| ATOM | 520 | O | LYS | H | 64 | 1.777 | −11.730 | 10.402 | 1.00 | 49.31 | O |
| ATOM | 521 | N | GLY | H | 65 | 3.477 | −10.455 | 11.107 | 1.00 | 48.75 | N |
| ATOM | 522 | CA | GLY | H | 65 | 2.599 | −9.301 | 11.282 | 1.00 | 48.94 | C |
| ATOM | 523 | C | GLY | H | 65 | 1.640 | −9.423 | 12.462 | 1.00 | 48.40 | C |
| ATOM | 524 | O | GLY | H | 65 | 0.720 | −8.621 | 12.605 | 1.00 | 48.62 | O |
| ATOM | 525 | N | GLN | H | 66 | 1.831 | −10.457 | 13.276 | 1.00 | 47.60 | N |
| ATOM | 526 | CA | GLN | H | 66 | 1.345 | −10.451 | 14.643 | 1.00 | 47.18 | C |
| ATOM | 527 | CB | GLN | H | 66 | 0.465 | −11.675 | 14.940 | 1.00 | 47.55 | C |
| ATOM | 528 | CG | GLN | H | 66 | −0.593 | −11.995 | 13.861 | 1.00 | 49.86 | C |
| ATOM | 529 | CD | GLN | H | 66 | −1.513 | −10.819 | 13.566 | 1.00 | 54.17 | C |
| ATOM | 530 | OE1 | GLN | H | 66 | −1.904 | −10.072 | 14.472 | 1.00 | 55.73 | O |
| ATOM | 531 | NE2 | GLN | H | 66 | −1.870 | −10.651 | 12.291 | 1.00 | 55.68 | N |
| ATOM | 532 | C | GLN | H | 66 | 2.587 | −10.418 | 15.530 | 1.00 | 45.95 | C |
| ATOM | 533 | O | GLN | H | 66 | 2.674 | −9.617 | 16.445 | 1.00 | 45.78 | O |
| ATOM | 534 | N | ALA | H | 67 | 3.568 | −11.255 | 15.199 | 1.00 | 45.16 | N |
| ATOM | 535 | CA | ALA | H | 67 | 4.785 | −11.419 | 15.993 | 1.00 | 44.49 | C |
| ATOM | 536 | CB | ALA | H | 67 | 5.241 | −12.851 | 15.949 | 1.00 | 43.91 | C |
| ATOM | 537 | C | ALA | H | 67 | 5.931 | −10.514 | 15.554 | 1.00 | 44.22 | C |
| ATOM | 538 | O | ALA | H | 67 | 6.128 | −10.288 | 14.361 | 1.00 | 44.00 | O |
| ATOM | 539 | N | THR | H | 68 | 6.697 | −10.038 | 16.539 | 1.00 | 43.96 | N |
| ATOM | 540 | CA | THR | H | 68 | 7.943 | −9.289 | 16.328 | 1.00 | 43.49 | C |
| ATOM | 541 | CB | THR | H | 68 | 7.742 | −7.796 | 16.553 | 1.00 | 43.40 | C |
| ATOM | 542 | OG1 | THR | H | 68 | 6.590 | −7.366 | 15.825 | 1.00 | 42.88 | O |
| ATOM | 543 | CG2 | THR | H | 68 | 8.986 | −6.998 | 16.109 | 1.00 | 42.72 | C |
| ATOM | 544 | C | THR | H | 68 | 9.032 | −9.754 | 17.281 | 1.00 | 43.90 | C |
| ATOM | 545 | O | THR | H | 68 | 8.869 | −9.676 | 18.507 | 1.00 | 43.78 | O |
| ATOM | 546 | N | LEU | H | 69 | 10.135 | −10.237 | 16.708 | 1.00 | 43.83 | N |
| ATOM | 547 | CA | LEU | H | 69 | 11.313 | −10.576 | 17.469 | 1.00 | 44.21 | C |
| ATOM | 548 | CB | LEU | H | 69 | 11.986 | −11.838 | 16.923 | 1.00 | 43.82 | C |
| ATOM | 549 | CG | LEU | H | 69 | 11.149 | −13.101 | 16.640 | 1.00 | 43.95 | C |
| ATOM | 550 | CD1 | LEU | H | 69 | 12.063 | −14.279 | 16.410 | 1.00 | 42.23 | C |
| ATOM | 551 | CD2 | LEU | H | 69 | 10.155 | −13.449 | 17.743 | 1.00 | 42.94 | C |
| ATOM | 552 | C | LEU | H | 69 | 12.273 | −9.398 | 17.422 | 1.00 | 44.96 | C |
| ATOM | 553 | O | LEU | H | 69 | 12.444 | −8.766 | 16.377 | 1.00 | 44.97 | O |
| ATOM | 554 | N | SER | H | 70 | 12.855 | −9.078 | 18.574 | 1.00 | 45.61 | N |
| ATOM | 555 | CA | SER | H | 70 | 13.873 | −8.036 | 18.688 | 1.00 | 46.35 | C |
| ATOM | 556 | CB | SER | H | 70 | 13.256 | −6.670 | 18.990 | 1.00 | 46.19 | C |
| ATOM | 557 | OG | SER | H | 70 | 12.494 | −6.714 | 20.177 | 1.00 | 45.96 | O |
| ATOM | 558 | C | SER | H | 70 | 14.884 | −8.433 | 19.757 | 1.00 | 47.31 | C |
| ATOM | 559 | O | SER | H | 70 | 14.676 | −9.406 | 20.494 | 1.00 | 46.80 | O |
| ATOM | 560 | N | ALA | H | 71 | 15.991 | −7.698 | 19.826 | 1.00 | 48.24 | N |
| ATOM | 561 | CA | ALA | H | 71 | 17.046 | −8.009 | 20.782 | 1.00 | 49.43 | C |
| ATOM | 562 | CB | ALA | H | 71 | 18.124 | −8.832 | 20.124 | 1.00 | 49.07 | C |
| ATOM | 563 | C | ALA | H | 71 | 17.631 | −6.730 | 21.360 | 1.00 | 50.67 | C |
| ATOM | 564 | O | ALA | H | 71 | 17.902 | −5.781 | 20.616 | 1.00 | 50.71 | O |
| ATOM | 565 | N | ASP | H | 72 | 17.789 | −6.692 | 22.686 | 1.00 | 51.71 | N |
| ATOM | 566 | CA | ASP | H | 72 | 18.559 | −5.618 | 23.321 | 1.00 | 52.46 | C |
| ATOM | 567 | CB | ASP | H | 72 | 17.890 | −5.075 | 24.587 | 1.00 | 52.83 | C |
| ATOM | 568 | CG | ASP | H | 72 | 18.702 | −3.949 | 25.229 | 1.00 | 53.95 | C |
| ATOM | 569 | OD1 | ASP | H | 72 | 19.322 | −4.179 | 26.292 | 1.00 | 53.26 | O |
| ATOM | 570 | OD2 | ASP | H | 72 | 18.757 | −2.847 | 24.636 | 1.00 | 54.91 | O |
| ATOM | 571 | C | ASP | H | 72 | 19.964 | −6.113 | 23.625 | 1.00 | 52.48 | C |
| ATOM | 572 | O | ASP | H | 72 | 20.197 | −6.776 | 24.640 | 1.00 | 52.58 | O |
| ATOM | 573 | N | LYS | H | 73 | 20.889 | −5.790 | 22.721 | 1.00 | 52.79 | N |
| ATOM | 574 | CA | LYS | H | 73 | 22.276 | −6.247 | 22.793 | 1.00 | 53.12 | C |
| ATOM | 575 | CB | LYS | H | 73 | 23.042 | −5.861 | 21.518 | 1.00 | 53.35 | C |
| ATOM | 576 | CG | LYS | H | 73 | 22.563 | −6.584 | 20.250 | 1.00 | 53.80 | C |
| ATOM | 577 | CD | LYS | H | 73 | 23.623 | −6.632 | 19.130 | 1.00 | 54.53 | C |
| ATOM | 578 | CE | LYS | H | 73 | 23.934 | −5.256 | 18.514 | 1.00 | 55.72 | C |
| ATOM | 579 | NZ | LYS | H | 73 | 22.757 | −4.561 | 17.905 | 1.00 | 55.68 | N |
| ATOM | 580 | C | LYS | H | 73 | 22.993 | −5.710 | 24.031 | 1.00 | 53.26 | C |
| ATOM | 581 | O | LYS | H | 73 | 23.977 | −6.291 | 24.476 | 1.00 | 53.36 | O |
| ATOM | 582 | N | SER | H | 74 | 22.488 | −4.613 | 24.591 | 1.00 | 53.39 | N |
| ATOM | 583 | CA | SER | H | 74 | 23.089 | −4.022 | 25.791 | 1.00 | 53.68 | C |
| ATOM | 584 | CB | SER | H | 74 | 22.720 | −2.539 | 25.928 | 1.00 | 53.77 | C |
| ATOM | 585 | OG | SER | H | 74 | 21.346 | −2.362 | 26.243 | 1.00 | 55.38 | O |
| ATOM | 586 | C | SER | H | 74 | 22.788 | −4.788 | 27.091 | 1.00 | 53.35 | C |
| ATOM | 587 | O | SER | H | 74 | 23.650 | −4.863 | 27.975 | 1.00 | 53.49 | O |
| ATOM | 588 | N | SER | H | 75 | 21.588 | −5.372 | 27.198 | 1.00 | 52.57 | N |
| ATOM | 589 | CA | SER | H | 75 | 21.199 | −6.133 | 28.404 | 1.00 | 51.19 | C |
| ATOM | 590 | CB | SER | H | 75 | 19.868 | −5.624 | 28.971 | 1.00 | 51.46 | C |
| ATOM | 591 | OG | SER | H | 75 | 18.802 | −5.805 | 28.046 | 1.00 | 51.30 | O |
| ATOM | 592 | C | SER | H | 75 | 21.140 | −7.650 | 28.219 | 1.00 | 50.41 | C |
| ATOM | 593 | O | SER | H | 75 | 20.714 | −8.368 | 29.127 | 1.00 | 50.10 | O |
| ATOM | 594 | N | SER | H | 76 | 21.582 | −8.138 | 27.061 | 1.00 | 48.96 | N |
| ATOM | 595 | CA | SER | H | 76 | 21.500 | −9.562 | 26.728 | 1.00 | 47.68 | C |
| ATOM | 596 | CB | SER | H | 76 | 22.475 | −10.379 | 27.581 | 1.00 | 48.18 | C |
| ATOM | 597 | OG | SER | H | 76 | 23.772 | −10.391 | 27.010 | 1.00 | 49.34 | O |
| ATOM | 598 | C | SER | H | 76 | 20.070 | −10.138 | 26.821 | 1.00 | 46.34 | C |
| ATOM | 599 | O | SER | H | 76 | 19.876 | −11.274 | 27.253 | 1.00 | 45.41 | O |
| ATOM | 600 | N | THR | H | 77 | 19.086 | −9.341 | 26.412 | 1.00 | 45.25 | N |
| ATOM | 601 | CA | THR | H | 77 | 17.685 | −9.732 | 26.503 | 1.00 | 45.18 | C |
| ATOM | 602 | CB | THR | H | 77 | 16.893 | −8.799 | 27.464 | 1.00 | 45.29 | C |
| ATOM | 603 | OG1 | THR | H | 77 | 17.539 | −8.780 | 28.743 | 1.00 | 45.46 | O |
| ATOM | 604 | CG2 | THR | H | 77 | 15.452 | −9.281 | 27.653 | 1.00 | 44.87 | C |
| ATOM | 605 | C | THR | H | 77 | 17.024 | −9.781 | 25.117 | 1.00 | 44.86 | C |
| ATOM | 606 | O | THR | H | 77 | 17.132 | −8.836 | 24.337 | 1.00 | 45.09 | O |
| ATOM | 607 | N | ALA | H | 78 | 16.366 | −10.905 | 24.825 | 1.00 | 44.40 | N |
| ATOM | 608 | CA | ALA | H | 78 | 15.551 | −11.081 | 23.612 | 1.00 | 43.61 | C |
| ATOM | 609 | CB | ALA | H | 78 | 15.733 | −12.493 | 23.068 | 1.00 | 43.32 | C |
| ATOM | 610 | C | ALA | H | 78 | 14.083 | −10.821 | 23.942 | 1.00 | 43.25 | C |
| ATOM | 611 | O | ALA | H | 78 | 13.623 | −11.177 | 25.028 | 1.00 | 43.44 | O |
| ATOM | 612 | N | TYR | H | 79 | 13.357 | −10.188 | 23.024 | 1.00 | 42.67 | N |
| ATOM | 613 | CA | TYR | H | 79 | 11.943 | −9.876 | 23.226 | 1.00 | 42.31 | C |
| ATOM | 614 | CB | TYR | H | 79 | 11.722 | −8.360 | 23.246 | 1.00 | 42.90 | C |
| ATOM | 615 | CG | TYR | H | 79 | 12.536 | −7.651 | 24.315 | 1.00 | 44.93 | C |
| ATOM | 616 | CD1 | TYR | H | 79 | 12.040 | −7.511 | 25.617 | 1.00 | 46.60 | C |
| ATOM | 617 | CE1 | TYR | H | 79 | 12.783 | −6.875 | 26.610 | 1.00 | 48.59 | C |
| ATOM | 618 | CZ | TYR | H | 79 | 14.044 | −6.380 | 26.303 | 1.00 | 48.91 | C |
| ATOM | 619 | OH | TYR | H | 79 | 14.782 | −5.748 | 27.281 | 1.00 | 51.42 | O |
| ATOM | 620 | CE2 | TYR | H | 79 | 14.558 | −6.507 | 25.021 | 1.00 | 48.52 | C |
| ATOM | 621 | CD2 | TYR | H | 79 | 13.801 | −7.144 | 24.032 | 1.00 | 45.88 | C |
| ATOM | 622 | C | TYR | H | 79 | 11.049 | −10.507 | 22.157 | 1.00 | 41.98 | C |
| ATOM | 623 | O | TYR | H | 79 | 11.470 | −10.660 | 21.013 | 1.00 | 41.96 | O |
| ATOM | 624 | N | LEU | H | 80 | 9.825 | −10.867 | 22.545 | 1.00 | 40.98 | N |
| ATOM | 625 | CA | LEU | H | 80 | 8.769 | −11.257 | 21.612 | 1.00 | 40.34 | C |
| ATOM | 626 | CB | LEU | H | 80 | 8.338 | −12.724 | 21.823 | 1.00 | 39.93 | C |
| ATOM | 627 | CG | LEU | H | 80 | 7.040 | −13.209 | 21.145 | 1.00 | 39.10 | C |
| ATOM | 628 | CD1 | LEU | H | 80 | 7.180 | −13.258 | 19.624 | 1.00 | 37.55 | C |
| ATOM | 629 | CD2 | LEU | H | 80 | 6.603 | −14.582 | 21.667 | 1.00 | 37.51 | C |
| ATOM | 630 | C | LEU | H | 80 | 7.575 | −10.338 | 21.800 | 1.00 | 40.27 | C |
| ATOM | 631 | O | LEU | H | 80 | 6.917 | −10.368 | 22.842 | 1.00 | 40.24 | O |
| ATOM | 632 | N | GLN | H | 81 | 7.298 | −9.509 | 20.802 | 1.00 | 40.40 | N |
| ATOM | 633 | CA | GLN | H | 81 | 6.107 | −8.684 | 20.847 | 1.00 | 40.85 | C |
| ATOM | 634 | CB | GLN | H | 81 | 6.369 | −7.315 | 20.252 | 1.00 | 41.25 | C |
| ATOM | 635 | CG | GLN | H | 81 | 7.296 | −6.453 | 21.054 | 1.00 | 43.01 | C |
| ATOM | 636 | CD | GLN | H | 81 | 7.647 | −5.203 | 20.296 | 1.00 | 45.26 | C |
| ATOM | 637 | OE1 | GLN | H | 81 | 7.021 | −4.167 | 20.487 | 1.00 | 46.73 | O |
| ATOM | 638 | NE2 | GLN | H | 81 | 8.623 | −5.301 | 19.395 | 1.00 | 44.19 | N |
| ATOM | 639 | C | GLN | H | 81 | 5.037 | −9.345 | 20.020 | 1.00 | 40.78 | C |
| ATOM | 640 | O | GLN | H | 81 | 5.351 | −10.023 | 19.050 | 1.00 | 40.28 | O |
| ATOM | 641 | N | TRP | H | 82 | 3.775 | −9.109 | 20.377 | 1.00 | 40.86 | N |
| ATOM | 642 | CA | TRP | H | 82 | 2.657 | −9.741 | 19.687 | 1.00 | 41.59 | C |
| ATOM | 643 | CB | TRP | H | 82 | 2.359 | −11.101 | 20.332 | 1.00 | 40.96 | C |
| ATOM | 644 | CG | TRP | H | 82 | 1.537 | −12.026 | 19.492 | 1.00 | 40.50 | C |
| ATOM | 645 | CD1 | TRP | H | 82 | 0.232 | −11.850 | 19.082 | 1.00 | 42.19 | C |
| ATOM | 646 | NE1 | TRP | H | 82 | −0.187 | −12.933 | 18.332 | 1.00 | 40.06 | N |
| ATOM | 647 | CE2 | TRP | H | 82 | 0.848 | −13.828 | 18.245 | 1.00 | 39.92 | C |
| ATOM | 648 | CD2 | TRP | H | 82 | 1.949 | −13.287 | 18.965 | 1.00 | 39.66 | C |
| ATOM | 649 | CE3 | TRP | H | 82 | 3.142 | −14.021 | 19.031 | 1.00 | 38.58 | C |
| ATOM | 650 | CZ3 | TRP | H | 82 | 3.200 | −15.257 | 18.385 | 1.00 | 39.43 | C |
| ATOM | 651 | CH2 | TRP | H | 82 | 2.090 | −15.768 | 17.690 | 1.00 | 37.78 | C |
| ATOM | 652 | CZ2 | TRP | H | 82 | 0.911 | −15.068 | 17.603 | 1.00 | 38.08 | C |
| ATOM | 653 | C | TRP | H | 82 | 1.424 | −8.865 | 19.776 | 1.00 | 42.34 | C |
| ATOM | 654 | O | TRP | H | 82 | 0.911 | −8.651 | 20.874 | 1.00 | 42.98 | O |
| ATOM | 655 | N | SER | H | 82A | 0.938 | −8.367 | 18.636 | 1.00 | 42.91 | N |
| ATOM | 656 | CA | SER | H | 82A | −0.287 | −7.553 | 18.621 | 1.00 | 42.95 | C |
| ATOM | 657 | CB | SER | H | 82A | −0.109 | −6.326 | 17.726 | 1.00 | 43.17 | C |
| ATOM | 658 | OG | SER | H | 82A | 0.535 | −6.687 | 16.517 | 1.00 | 43.81 | O |
| ATOM | 659 | C | SER | H | 82A | −1.541 | −8.347 | 18.225 | 1.00 | 43.09 | C |
| ATOM | 660 | O | SER | H | 82A | −1.444 | −9.498 | 17.791 | 1.00 | 43.21 | O |
| ATOM | 661 | N | SER | H | 82B | −2.711 | −7.718 | 18.386 | 1.00 | 42.84 | N |
| ATOM | 662 | CA | SER | H | 82B | −4.025 | −8.353 | 18.175 | 1.00 | 42.81 | C |
| ATOM | 663 | CB | SER | H | 82B | −4.466 | −8.246 | 16.714 | 1.00 | 42.93 | C |
| ATOM | 664 | OG | SER | H | 82B | −4.200 | −6.953 | 16.215 | 1.00 | 43.86 | O |
| ATOM | 665 | C | SER | H | 82B | −4.085 | −9.811 | 18.630 | 1.00 | 42.31 | C |
| ATOM | 666 | O | SER | H | 82B | −4.401 | −10.706 | 17.837 | 1.00 | 42.79 | O |
| ATOM | 667 | N | LEU | H | 82C | −3.799 | −10.054 | 19.907 | 1.00 | 41.41 | N |
| ATOM | 668 | CA | LEU | H | 82C | −3.846 | −11.418 | 20.424 | 1.00 | 40.23 | C |
| ATOM | 669 | CB | LEU | H | 82C | −3.489 | −11.447 | 21.913 | 1.00 | 40.28 | C |
| ATOM | 670 | CG | LEU | H | 82C | −2.036 | −11.173 | 22.344 | 1.00 | 39.93 | C |
| ATOM | 671 | CD1 | LEU | H | 82C | −1.976 | −10.642 | 23.791 | 1.00 | 37.29 | C |
| ATOM | 672 | CD2 | LEU | H | 82C | −1.177 | −12.415 | 22.198 | 1.00 | 38.69 | C |
| ATOM | 673 | C | LEU | H | 82C | −5.223 | −12.042 | 20.178 | 1.00 | 39.57 | C |
| ATOM | 674 | O | LEU | H | 82C | −6.243 | −11.376 | 20.275 | 1.00 | 39.07 | O |
| ATOM | 675 | N | LYS | H | 83 | −5.239 | −13.323 | 19.847 | 1.00 | 39.11 | N |
| ATOM | 676 | CA | LYS | H | 83 | −6.481 | −14.058 | 19.683 | 1.00 | 38.75 | C |
| ATOM | 677 | CB | LYS | H | 83 | −6.496 | −14.769 | 18.320 | 1.00 | 39.42 | C |
| ATOM | 678 | CG | LYS | H | 83 | −6.272 | −13.865 | 17.121 | 1.00 | 41.14 | C |
| ATOM | 679 | CD | LYS | H | 83 | −5.531 | −14.614 | 15.995 | 1.00 | 45.31 | C |
| ATOM | 680 | CE | LYS | H | 83 | −6.434 | −15.633 | 15.308 | 1.00 | 48.28 | C |
| ATOM | 681 | NZ | LYS | H | 83 | −5.894 | −16.040 | 13.976 | 1.00 | 49.89 | N |
| ATOM | 682 | C | LYS | H | 83 | −6.528 | −15.094 | 20.796 | 1.00 | 37.63 | C |
| ATOM | 683 | O | LYS | H | 83 | −5.486 | −15.434 | 21.356 | 1.00 | 37.89 | O |
| ATOM | 684 | N | ALA | H | 84 | −7.709 | −15.615 | 21.105 | 1.00 | 36.65 | N |
| ATOM | 685 | CA | ALA | H | 84 | −7.816 | −16.666 | 22.123 | 1.00 | 36.12 | C |
| ATOM | 686 | CB | ALA | H | 84 | −9.272 | −17.105 | 22.318 | 1.00 | 35.91 | C |
| ATOM | 687 | C | ALA | H | 84 | −6.931 | −17.883 | 21.825 | 1.00 | 35.63 | C |
| ATOM | 688 | O | ALA | H | 84 | −6.443 | −18.538 | 22.746 | 1.00 | 35.87 | O |
| ATOM | 689 | N | SER | H | 85 | −6.729 | −18.181 | 20.542 | 1.00 | 34.43 | N |
| ATOM | 690 | CA | SER | H | 85 | −5.901 | −19.314 | 20.141 | 1.00 | 33.75 | C |
| ATOM | 691 | CB | SER | H | 85 | −6.163 | −19.670 | 18.669 | 1.00 | 32.77 | C |
| ATOM | 692 | OG | SER | H | 85 | −5.727 | −18.609 | 17.849 | 1.00 | 31.98 | O |
| ATOM | 693 | C | SER | H | 85 | −4.395 | −19.069 | 20.381 | 1.00 | 33.22 | C |
| ATOM | 694 | O | SER | H | 85 | −3.588 | −19.944 | 20.127 | 1.00 | 32.37 | O |
| ATOM | 695 | N | ASP | H | 86 | −4.029 | −17.879 | 20.860 | 1.00 | 33.42 | N |
| ATOM | 696 | CA | ASP | H | 86 | −2.632 | −17.578 | 21.192 | 1.00 | 32.81 | C |
| ATOM | 697 | CB | ASP | H | 86 | −2.287 | −16.094 | 20.944 | 1.00 | 33.48 | C |
| ATOM | 698 | CG | ASP | H | 86 | −2.406 | −15.680 | 19.475 | 1.00 | 35.35 | C |
| ATOM | 699 | OD1 | ASP | H | 86 | −2.852 | −14.535 | 19.222 | 1.00 | 35.04 | O |
| ATOM | 700 | OD2 | ASP | H | 86 | −2.036 | −16.473 | 18.573 | 1.00 | 37.74 | O |
| ATOM | 701 | C | ASP | H | 86 | −2.336 | −17.943 | 22.642 | 1.00 | 32.42 | C |
| ATOM | 702 | O | ASP | H | 86 | −1.220 | −17.703 | 23.135 | 1.00 | 31.80 | O |
| ATOM | 703 | N | THR | H | 87 | −3.341 | −18.502 | 23.329 | 1.00 | 31.61 | N |
| ATOM | 704 | CA | THR | H | 87 | −3.180 | −19.063 | 24.679 | 1.00 | 29.92 | C |
| ATOM | 705 | CB | THR | H | 87 | −4.542 | −19.519 | 25.285 | 1.00 | 29.76 | C |
| ATOM | 706 | OG1 | THR | H | 87 | −5.389 | −18.377 | 25.441 | 1.00 | 29.66 | O |
| ATOM | 707 | CG2 | THR | H | 87 | −4.351 | −20.183 | 26.642 | 1.00 | 28.04 | C |
| ATOM | 708 | C | THR | H | 87 | −2.230 | −20.248 | 24.642 | 1.00 | 29.91 | C |
| ATOM | 709 | O | THR | H | 87 | −2.525 | −21.292 | 24.028 | 1.00 | 29.61 | O |
| ATOM | 710 | N | ALA | H | 88 | −1.087 | −20.098 | 25.315 | 1.00 | 29.01 | N |
| ATOM | 711 | CA | ALA | H | 88 | −0.021 | −21.093 | 25.224 | 1.00 | 29.18 | C |
| ATOM | 712 | CB | ALA | H | 88 | 0.559 | −21.111 | 23.798 | 1.00 | 28.20 | C |
| ATOM | 713 | C | ALA | H | 88 | 1.088 | −20.824 | 26.248 | 1.00 | 29.33 | C |
| ATOM | 714 | O | ALA | H | 88 | 1.221 | −19.718 | 26.758 | 1.00 | 29.25 | O |
| ATOM | 715 | N | MET | H | 89 | 1.879 | −21.842 | 26.546 | 1.00 | 30.03 | N |
| ATOM | 716 | CA | MET | H | 89 | 3.135 | −21.620 | 27.267 | 1.00 | 30.90 | C |
| ATOM | 717 | CB | MET | H | 89 | 3.553 | −22.904 | 27.981 | 1.00 | 31.36 | C |
| ATOM | 718 | CG | MET | H | 89 | 4.881 | −22.818 | 28.763 | 1.00 | 33.43 | C |
| ATOM | 719 | SD | MET | H | 89 | 4.785 | −21.723 | 30.184 | 1.00 | 35.89 | S |
| ATOM | 720 | CE | MET | H | 89 | 3.818 | −22.696 | 31.334 | 1.00 | 35.18 | C |
| ATOM | 721 | C | MET | H | 89 | 4.197 | −21.208 | 26.234 | 1.00 | 30.58 | C |
| ATOM | 722 | O | MET | H | 89 | 4.509 | −21.984 | 25.343 | 1.00 | 29.79 | O |
| ATOM | 723 | N | TYR | H | 90 | 4.730 | −19.993 | 26.351 | 1.00 | 30.76 | N |
| ATOM | 724 | CA | TYR | H | 90 | 5.789 | −19.502 | 25.448 | 1.00 | 30.81 | C |
| ATOM | 725 | CB | TYR | H | 90 | 5.580 | −18.033 | 25.112 | 1.00 | 30.53 | C |
| ATOM | 726 | CG | TYR | H | 90 | 4.375 | −17.779 | 24.240 | 1.00 | 31.96 | C |
| ATOM | 727 | CD1 | TYR | H | 90 | 3.074 | −17.980 | 24.726 | 1.00 | 31.12 | C |
| ATOM | 728 | CE1 | TYR | H | 90 | 1.969 | −17.744 | 23.930 | 1.00 | 29.99 | C |
| ATOM | 729 | CZ | TYR | H | 90 | 2.149 | −17.306 | 22.630 | 1.00 | 33.04 | C |
| ATOM | 730 | OH | TYR | H | 90 | 1.046 | −17.076 | 21.829 | 1.00 | 32.69 | O |
| ATOM | 731 | CE2 | TYR | H | 90 | 3.431 | −17.080 | 22.125 | 1.00 | 30.65 | C |
| ATOM | 732 | CD2 | TYR | H | 90 | 4.527 | −17.331 | 22.920 | 1.00 | 31.50 | C |
| ATOM | 733 | C | TYR | H | 90 | 7.217 | −19.719 | 25.980 | 1.00 | 31.06 | C |
| ATOM | 734 | O | TYR | H | 90 | 7.556 | −19.305 | 27.099 | 1.00 | 30.71 | O |
| ATOM | 735 | N | PHE | H | 91 | 8.038 | −20.368 | 25.156 | 1.00 | 30.54 | N |
| ATOM | 736 | CA | PHE | H | 91 | 9.424 | −20.671 | 25.489 | 1.00 | 30.74 | C |
| ATOM | 737 | CB | PHE | H | 91 | 9.682 | −22.161 | 25.281 | 1.00 | 30.88 | C |
| ATOM | 738 | CG | PHE | H | 91 | 9.033 | −23.039 | 26.308 | 1.00 | 31.24 | C |
| ATOM | 739 | CD1 | PHE | H | 91 | 9.593 | −23.166 | 27.586 | 1.00 | 31.21 | C |
| ATOM | 740 | CE1 | PHE | H | 91 | 9.006 | −23.989 | 28.565 | 1.00 | 32.98 | C |
| ATOM | 741 | CZ | PHE | H | 91 | 7.835 | −24.694 | 28.261 | 1.00 | 33.21 | C |
| ATOM | 742 | CE2 | PHE | H | 91 | 7.261 | −24.566 | 26.966 | 1.00 | 33.43 | C |
| ATOM | 743 | CD2 | PHE | H | 91 | 7.864 | −23.730 | 26.011 | 1.00 | 31.25 | C |
| ATOM | 744 | C | PHE | H | 91 | 10.397 | −19.899 | 24.610 | 1.00 | 31.44 | C |
| ATOM | 745 | O | PHE | H | 91 | 10.147 | −19.700 | 23.396 | 1.00 | 31.03 | O |
| ATOM | 746 | N | CYS | H | 92 | 11.506 | −19.449 | 25.200 | 1.00 | 30.78 | N |
| ATOM | 747 | CA | CYS | H | 92 | 12.634 | −19.070 | 24.369 | 1.00 | 30.62 | C |
| ATOM | 748 | CB | CYS | H | 92 | 13.191 | −17.687 | 24.698 | 1.00 | 31.21 | C |
| ATOM | 749 | SG | CYS | H | 92 | 13.814 | −17.527 | 26.361 | 1.00 | 34.93 | S |
| ATOM | 750 | C | CYS | H | 92 | 13.650 | −20.178 | 24.529 | 1.00 | 30.19 | C |
| ATOM | 751 | O | CYS | H | 92 | 13.635 | −20.899 | 25.539 | 1.00 | 29.98 | O |
| ATOM | 752 | N | ALA | H | 93 | 14.483 | −20.373 | 23.515 | 1.00 | 29.10 | N |
| ATOM | 753 | CA | ALA | H | 93 | 15.430 | −21.473 | 23.554 | 1.00 | 30.09 | C |
| ATOM | 754 | CB | ALA | H | 93 | 14.757 | −22.790 | 23.161 | 1.00 | 29.48 | C |
| ATOM | 755 | C | ALA | H | 93 | 16.654 | −21.223 | 22.668 | 1.00 | 30.53 | C |
| ATOM | 756 | O | ALA | H | 93 | 16.579 | −20.517 | 21.665 | 1.00 | 30.49 | O |
| ATOM | 757 | N | ARG | H | 94 | 17.776 | −21.804 | 23.052 | 1.00 | 30.97 | N |
| ATOM | 758 | CA | ARG | H | 94 | 18.927 | −21.818 | 22.171 | 1.00 | 31.60 | C |
| ATOM | 759 | CB | ARG | H | 94 | 20.128 | −21.128 | 22.814 | 1.00 | 32.30 | C |
| ATOM | 760 | CG | ARG | H | 94 | 21.367 | −21.105 | 21.910 | 1.00 | 32.81 | C |
| ATOM | 761 | CD | ARG | H | 94 | 22.629 | −20.875 | 22.730 | 1.00 | 34.55 | C |
| ATOM | 762 | NE | ARG | H | 94 | 23.778 | −20.646 | 21.849 | 1.00 | 35.54 | N |
| ATOM | 763 | CZ | ARG | H | 94 | 25.034 | −20.507 | 22.271 | 1.00 | 37.16 | C |
| ATOM | 764 | NH1 | ARG | H | 94 | 26.011 | −20.309 | 21.397 | 1.00 | 37.24 | N |
| ATOM | 765 | NH2 | ARG | H | 94 | 25.315 | −20.581 | 23.564 | 1.00 | 35.74 | N |
| ATOM | 766 | C | ARG | H | 94 | 19.235 | −23.265 | 21.916 | 1.00 | 31.28 | C |
| ATOM | 767 | O | ARG | H | 94 | 19.580 | −23.999 | 22.831 | 1.00 | 31.47 | O |
| ATOM | 768 | N | ARG | H | 95 | 19.093 | −23.693 | 20.669 | 1.00 | 30.64 | N |
| ATOM | 769 | CA | ARG | H | 95 | 19.282 | −25.104 | 20.382 | 1.00 | 30.48 | C |
| ATOM | 770 | CB | ARG | H | 95 | 17.994 | −25.717 | 19.831 | 1.00 | 30.42 | C |
| ATOM | 771 | CG | ARG | H | 95 | 16.704 | −25.317 | 20.570 | 1.00 | 30.52 | C |
| ATOM | 772 | CD | ARG | H | 95 | 15.559 | −26.186 | 20.075 | 1.00 | 30.54 | C |
| ATOM | 773 | NE | ARG | H | 95 | 15.019 | −25.742 | 18.786 | 1.00 | 29.98 | N |
| ATOM | 774 | CZ | ARG | H | 95 | 14.335 | −26.523 | 17.950 | 1.00 | 28.90 | C |
| ATOM | 775 | NH1 | ARG | H | 95 | 14.135 | −27.799 | 18.223 | 1.00 | 28.77 | N |
| ATOM | 776 | NH2 | ARG | H | 95 | 13.858 | −26.028 | 16.825 | 1.00 | 29.93 | N |
| ATOM | 777 | C | ARG | H | 95 | 20.379 | −25.363 | 19.381 | 1.00 | 30.56 | C |
| ATOM | 778 | O | ARG | H | 95 | 20.980 | −26.441 | 19.392 | 1.00 | 30.22 | O |
| ATOM | 779 | N | PHE | H | 96 | 20.586 | −24.407 | 18.477 | 1.00 | 30.66 | N |
| ATOM | 780 | CA | PHE | H | 96 | 21.457 | −24.655 | 17.324 | 1.00 | 31.47 | C |
| ATOM | 781 | CB | PHE | H | 96 | 20.637 | −24.674 | 16.019 | 1.00 | 30.76 | C |
| ATOM | 782 | CG | PHE | H | 96 | 19.439 | −25.585 | 16.057 | 1.00 | 28.28 | C |
| ATOM | 783 | CD1 | PHE | H | 96 | 19.576 | −26.920 | 16.364 | 1.00 | 27.89 | C |
| ATOM | 784 | CE1 | PHE | H | 96 | 18.461 | −27.770 | 16.390 | 1.00 | 30.66 | C |
| ATOM | 785 | CZ | PHE | H | 96 | 17.193 | −27.259 | 16.083 | 1.00 | 27.73 | C |
| ATOM | 786 | CE2 | PHE | H | 96 | 17.060 | −25.926 | 15.762 | 1.00 | 26.33 | C |
| ATOM | 787 | CD2 | PHE | H | 96 | 18.174 | −25.099 | 15.739 | 1.00 | 28.89 | C |
| ATOM | 788 | C | PHE | H | 96 | 22.561 | −23.614 | 17.212 | 1.00 | 32.08 | C |
| ATOM | 789 | O | PHE | H | 96 | 22.288 | −22.416 | 17.193 | 1.00 | 32.29 | O |
| ATOM | 790 | N | GLY | H | 97 | 23.806 | −24.074 | 17.147 | 1.00 | 32.94 | N |
| ATOM | 791 | CA | GLY | H | 97 | 24.884 | −23.195 | 16.713 | 1.00 | 33.94 | C |
| ATOM | 792 | C | GLY | H | 97 | 24.760 | −22.979 | 15.205 | 1.00 | 34.69 | C |
| ATOM | 793 | O | GLY | H | 97 | 23.997 | −23.698 | 14.523 | 1.00 | 34.41 | O |
| ATOM | 794 | N | TYR | H | 98 | 25.498 | −21.995 | 14.693 | 1.00 | 35.41 | N |
| ATOM | 795 | CA | TYR | H | 98 | 25.561 | −21.704 | 13.250 | 1.00 | 36.63 | C |
| ATOM | 796 | CB | TYR | H | 98 | 26.739 | −20.766 | 12.905 | 1.00 | 36.99 | C |
| ATOM | 797 | CG | TYR | H | 98 | 26.671 | −19.397 | 13.534 | 1.00 | 39.60 | C |
| ATOM | 798 | CD1 | TYR | H | 98 | 27.735 | −18.899 | 14.286 | 1.00 | 41.77 | C |
| ATOM | 799 | CE1 | TYR | H | 98 | 27.663 | −17.630 | 14.881 | 1.00 | 42.91 | C |
| ATOM | 800 | CZ | TYR | H | 98 | 26.518 | −16.856 | 14.710 | 1.00 | 43.47 | C |
| ATOM | 801 | OH | TYR | H | 98 | 26.417 | −15.595 | 15.277 | 1.00 | 44.50 | O |
| ATOM | 802 | CE2 | TYR | H | 98 | 25.457 | −17.338 | 13.970 | 1.00 | 42.13 | C |
| ATOM | 803 | CD2 | TYR | H | 98 | 25.540 | −18.597 | 13.385 | 1.00 | 42.09 | C |
| ATOM | 804 | C | TYR | H | 98 | 25.738 | −22.991 | 12.483 | 1.00 | 35.77 | C |
| ATOM | 805 | O | TYR | H | 98 | 26.509 | −23.846 | 12.892 | 1.00 | 35.80 | O |
| ATOM | 806 | N | TYR | H | 99 | 25.021 | −23.128 | 11.375 | 1.00 | 35.78 | N |
| ATOM | 807 | CA | TYR | H | 99 | 25.036 | −24.372 | 10.607 | 1.00 | 36.24 | C |
| ATOM | 808 | CB | TYR | H | 99 | 23.964 | −24.316 | 9.512 | 1.00 | 36.13 | C |
| ATOM | 809 | CG | TYR | H | 99 | 22.580 | −24.669 | 10.021 | 1.00 | 36.87 | C |
| ATOM | 810 | CD1 | TYR | H | 99 | 22.159 | −24.291 | 11.318 | 1.00 | 36.31 | C |
| ATOM | 811 | CE1 | TYR | H | 99 | 20.894 | −24.629 | 11.787 | 1.00 | 35.96 | C |
| ATOM | 812 | CZ | TYR | H | 99 | 20.041 | −25.344 | 10.952 | 1.00 | 36.25 | C |
| ATOM | 813 | OH | TYR | H | 99 | 18.793 | −25.693 | 11.386 | 1.00 | 37.56 | O |
| ATOM | 814 | CE2 | TYR | H | 99 | 20.428 | −25.715 | 9.672 | 1.00 | 35.78 | C |
| ATOM | 815 | CD2 | TYR | H | 99 | 21.688 | −25.378 | 9.216 | 1.00 | 35.83 | C |
| ATOM | 816 | C | TYR | H | 99 | 26.422 | −24.688 | 10.024 | 1.00 | 36.64 | C |
| ATOM | 817 | O | TYR | H | 99 | 27.166 | −23.790 | 9.640 | 1.00 | 36.47 | O |
| ATOM | 818 | N | GLY | H | 100 | 26.767 | −25.964 | 9.990 | 1.00 | 36.98 | N |
| ATOM | 819 | CA | GLY | H | 100 | 28.125 | −26.376 | 9.637 | 1.00 | 37.79 | C |
| ATOM | 820 | C | GLY | H | 100 | 29.061 | −26.465 | 10.832 | 1.00 | 37.64 | C |
| ATOM | 821 | O | GLY | H | 100 | 30.082 | −27.147 | 10.765 | 1.00 | 38.56 | O |
| ATOM | 822 | N | SER | H | 100A | 28.736 | −25.771 | 11.918 | 1.00 | 37.45 | N |
| ATOM | 823 | CA | SER | H | 100A | 29.612 | −25.750 | 13.116 | 1.00 | 37.67 | C |
| ATOM | 824 | CB | SER | H | 100A | 29.237 | −24.610 | 14.072 | 1.00 | 37.77 | C |
| ATOM | 825 | OG | SER | H | 100A | 27.995 | −24.868 | 14.732 | 1.00 | 37.13 | O |
| ATOM | 826 | C | SER | H | 100A | 29.652 | −27.078 | 13.881 | 1.00 | 37.65 | C |
| ATOM | 827 | O | SER | H | 100A | 30.658 | −27.402 | 14.531 | 1.00 | 37.67 | O |
| ATOM | 828 | N | GLY | H | 100B | 28.575 | −27.856 | 13.784 | 1.00 | 36.98 | N |
| ATOM | 829 | CA | GLY | H | 100B | 28.458 | −29.090 | 14.563 | 1.00 | 36.49 | C |
| ATOM | 830 | C | GLY | H | 100B | 28.048 | −28.835 | 16.010 | 1.00 | 36.28 | C |
| ATOM | 831 | O | GLY | H | 100B | 28.005 | −29.759 | 16.822 | 1.00 | 36.75 | O |
| ATOM | 832 | N | ASN | H | 100C | 27.725 | −27.588 | 16.327 | 1.00 | 35.75 | N |
| ATOM | 833 | CA | ASN | H | 100C | 27.411 | −27.198 | 17.702 | 1.00 | 35.96 | C |
| ATOM | 834 | CB | ASN | H | 100C | 28.076 | −25.867 | 18.058 | 1.00 | 35.44 | C |
| ATOM | 835 | CG | ASN | H | 100C | 29.590 | −25.991 | 18.175 | 1.00 | 34.92 | C |
| ATOM | 836 | OD1 | ASN | H | 100C | 30.114 | −27.065 | 18.469 | 1.00 | 33.65 | O |
| ATOM | 837 | ND2 | ASN | H | 100C | 30.294 | −24.893 | 17.937 | 1.00 | 35.22 | N |
| ATOM | 838 | C | ASN | H | 100C | 25.933 | −27.147 | 18.042 | 1.00 | 35.69 | C |
| ATOM | 839 | O | ASN | H | 100C | 25.140 | −26.476 | 17.377 | 1.00 | 35.71 | O |
| ATOM | 840 | N | TYR | H | 100D | 25.600 | −27.853 | 19.112 | 1.00 | 36.00 | N |
| ATOM | 841 | CA | TYR | H | 100D | 24.229 | −28.015 | 19.575 | 1.00 | 35.45 | C |
| ATOM | 842 | CB | TYR | H | 100D | 23.763 | −29.457 | 19.334 | 1.00 | 35.57 | C |
| ATOM | 843 | CG | TYR | H | 100D | 23.383 | −29.667 | 17.888 | 1.00 | 35.65 | C |
| ATOM | 844 | CD1 | TYR | H | 100D | 22.059 | −29.494 | 17.466 | 1.00 | 35.00 | C |
| ATOM | 845 | CE1 | TYR | H | 100D | 21.708 | −29.638 | 16.119 | 1.00 | 34.92 | C |
| ATOM | 846 | CZ | TYR | H | 100D | 22.691 | −29.946 | 15.181 | 1.00 | 35.45 | C |
| ATOM | 847 | OH | TYR | H | 100D | 22.338 | −30.089 | 13.858 | 1.00 | 35.18 | O |
| ATOM | 848 | CE2 | TYR | H | 100D | 24.015 | −30.104 | 15.576 | 1.00 | 34.49 | C |
| ATOM | 849 | CD2 | TYR | H | 100D | 24.353 | −29.970 | 16.925 | 1.00 | 35.14 | C |
| ATOM | 850 | C | TYR | H | 100D | 24.162 | −27.650 | 21.043 | 1.00 | 35.71 | C |
| ATOM | 851 | O | TYR | H | 100D | 25.091 | −27.927 | 21.814 | 1.00 | 33.96 | O |
| ATOM | 852 | N | PHE | H | 100E | 23.061 | −26.998 | 21.411 | 1.00 | 35.65 | N |
| ATOM | 853 | CA | PHE | H | 100E | 22.814 | −26.643 | 22.789 | 1.00 | 35.37 | C |
| ATOM | 854 | CB | PHE | H | 100E | 23.003 | −25.141 | 22.980 | 1.00 | 35.81 | C |
| ATOM | 855 | CG | PHE | H | 100E | 24.311 | −24.633 | 22.433 | 1.00 | 37.27 | C |
| ATOM | 856 | CD1 | PHE | H | 100E | 25.475 | −24.706 | 23.197 | 1.00 | 38.42 | C |
| ATOM | 857 | CE1 | PHE | H | 100E | 26.713 | −24.259 | 22.684 | 1.00 | 38.85 | C |
| ATOM | 858 | CZ | PHE | H | 100E | 26.773 | −23.732 | 21.406 | 1.00 | 39.70 | C |
| ATOM | 859 | CE2 | PHE | H | 100E | 25.603 | −23.664 | 20.615 | 1.00 | 41.06 | C |
| ATOM | 860 | CD2 | PHE | H | 100E | 24.386 | −24.113 | 21.133 | 1.00 | 38.95 | C |
| ATOM | 861 | C | PHE | H | 100E | 21.405 | −27.084 | 23.166 | 1.00 | 35.35 | C |
| ATOM | 862 | O | PHE | H | 100E | 20.678 | −27.647 | 22.345 | 1.00 | 35.09 | O |
| ATOM | 863 | N | ASP | H | 101 | 21.025 | −26.841 | 24.416 | 1.00 | 34.60 | N |
| ATOM | 864 | CA | ASP | H | 101 | 19.671 | −27.107 | 24.849 | 1.00 | 33.77 | C |
| ATOM | 865 | CB | ASP | H | 101 | 19.583 | −28.567 | 25.318 | 1.00 | 33.66 | C |
| ATOM | 866 | CG | ASP | H | 101 | 18.151 | −29.106 | 25.357 | 1.00 | 35.28 | C |
| ATOM | 867 | OD1 | ASP | H | 101 | 17.230 | −28.577 | 24.661 | 1.00 | 32.99 | O |
| ATOM | 868 | OD2 | ASP | H | 101 | 17.959 | −30.085 | 26.115 | 1.00 | 34.40 | O |
| ATOM | 869 | C | ASP | H | 101 | 19.322 | −26.123 | 25.960 | 1.00 | 33.09 | C |
| ATOM | 870 | O | ASP | H | 101 | 19.017 | −26.539 | 27.060 | 1.00 | 32.43 | O |
| ATOM | 871 | N | TYR | H | 102 | 19.408 | −24.819 | 25.696 | 1.00 | 32.31 | N |
| ATOM | 872 | CA | TYR | H | 102 | 18.995 | −23.847 | 26.713 | 1.00 | 32.23 | C |
| ATOM | 873 | CB | TYR | H | 102 | 19.734 | −22.497 | 26.639 | 1.00 | 33.07 | C |
| ATOM | 874 | CG | TYR | H | 102 | 21.260 | −22.502 | 26.629 | 1.00 | 34.10 | C |
| ATOM | 875 | CD1 | TYR | H | 102 | 21.993 | −23.672 | 26.822 | 1.00 | 37.76 | C |
| ATOM | 876 | CE1 | TYR | H | 102 | 23.396 | −23.664 | 26.807 | 1.00 | 41.02 | C |
| ATOM | 877 | CZ | TYR | H | 102 | 24.069 | −22.456 | 26.629 | 1.00 | 41.59 | C |
| ATOM | 878 | OH | TYR | H | 102 | 25.448 | −22.437 | 26.623 | 1.00 | 43.75 | O |
| ATOM | 879 | CE2 | TYR | H | 102 | 23.362 | −21.277 | 26.458 | 1.00 | 39.85 | C |
| ATOM | 880 | CD2 | TYR | H | 102 | 21.960 | −21.308 | 26.464 | 1.00 | 37.25 | C |
| ATOM | 881 | C | TYR | H | 102 | 17.534 | −23.538 | 26.502 | 1.00 | 32.09 | C |
| ATOM | 882 | O | TYR | H | 102 | 17.149 | −23.147 | 25.402 | 1.00 | 30.88 | O |
| ATOM | 883 | N | TRP | H | 103 | 16.742 | −23.688 | 27.565 | 1.00 | 30.96 | N |
| ATOM | 884 | CA | TRP | H | 103 | 15.345 | −23.325 | 27.552 | 1.00 | 30.64 | C |
| ATOM | 885 | CB | TRP | H | 103 | 14.446 | −24.565 | 27.721 | 1.00 | 30.34 | C |
| ATOM | 886 | CG | TRP | H | 103 | 14.540 | −25.506 | 26.545 | 1.00 | 29.09 | C |
| ATOM | 887 | CD1 | TRP | H | 103 | 15.553 | −26.388 | 26.279 | 1.00 | 26.48 | C |
| ATOM | 888 | NE1 | TRP | H | 103 | 15.285 | −27.069 | 25.112 | 1.00 | 28.91 | N |
| ATOM | 889 | CE2 | TRP | H | 103 | 14.093 | −26.625 | 24.601 | 1.00 | 26.45 | C |
| ATOM | 890 | CD2 | TRP | H | 103 | 13.593 | −25.643 | 25.479 | 1.00 | 26.03 | C |
| ATOM | 891 | CE3 | TRP | H | 103 | 12.360 | −25.034 | 25.188 | 1.00 | 26.23 | C |
| ATOM | 892 | CZ3 | TRP | H | 103 | 11.678 | −25.415 | 24.043 | 1.00 | 25.76 | C |
| ATOM | 893 | CH2 | TRP | H | 103 | 12.203 | −26.397 | 23.178 | 1.00 | 26.12 | C |
| ATOM | 894 | CZ2 | TRP | H | 103 | 13.405 | −27.014 | 23.441 | 1.00 | 26.81 | C |
| ATOM | 895 | C | TRP | H | 103 | 15.043 | −22.335 | 28.645 | 1.00 | 30.94 | C |
| ATOM | 896 | O | TRP | H | 103 | 15.452 | −22.502 | 29.796 | 1.00 | 31.26 | O |
| ATOM | 897 | N | GLY | H | 104 | 14.323 | −21.295 | 28.269 | 1.00 | 30.31 | N |
| ATOM | 898 | CA | GLY | H | 104 | 13.726 | −20.419 | 29.240 | 1.00 | 30.75 | C |
| ATOM | 899 | C | GLY | H | 104 | 12.766 | −21.221 | 30.115 | 1.00 | 30.58 | C |
| ATOM | 900 | O | GLY | H | 104 | 12.479 | −22.413 | 29.877 | 1.00 | 30.46 | O |
| ATOM | 901 | N | GLN | H | 105 | 12.186 | −20.743 | 31.507 | 1.00 | 31.55 | N |
| ATOM | 902 | CA | GLN | H | 105 | 11.327 | −21.583 | 32.354 | 1.00 | 30.89 | C |
| ATOM | 903 | CB | GLN | H | 105 | 11.339 | −21.049 | 33.796 | 1.00 | 30.18 | C |
| ATOM | 904 | CG | GLN | H | 105 | 10.594 | −19.721 | 34.019 | 1.00 | 27.63 | C |
| ATOM | 905 | CD | GLN | H | 105 | 11.365 | −18.490 | 33.583 | 1.00 | 28.07 | C |
| ATOM | 906 | OE1 | GLN | H | 105 | 12.301 | −18.565 | 32.789 | 1.00 | 28.99 | O |
| ATOM | 907 | NE2 | GLN | H | 105 | 10.966 | −17.337 | 34.096 | 1.00 | 27.20 | N |
| ATOM | 908 | C | GLN | H | 105 | 9.907 | −21.579 | 31.764 | 1.00 | 31.41 | C |
| ATOM | 909 | O | GLN | H | 105 | 9.050 | −22.359 | 32.189 | 1.00 | 31.48 | O |
| ATOM | 910 | N | GLY | H | 106 | 9.684 | −20.689 | 30.792 | 1.00 | 31.61 | N |
| ATOM | 911 | CA | GLY | H | 106 | 8.400 | −20.546 | 30.103 | 1.00 | 31.54 | C |
| ATOM | 912 | C | GLY | H | 106 | 7.630 | −19.351 | 30.624 | 1.00 | 32.12 | C |
| ATOM | 913 | O | GLY | H | 106 | 7.751 | −18.979 | 31.799 | 1.00 | 31.52 | O |
| ATOM | 914 | N | THR | H | 107 | 6.867 | −18.728 | 29.734 | 1.00 | 32.54 | N |
| ATOM | 915 | CA | THR | H | 107 | 5.943 | −17.672 | 30.105 | 1.00 | 33.86 | C |
| ATOM | 916 | CB | THR | H | 107 | 6.318 | −16.366 | 29.409 | 1.00 | 33.81 | C |
| ATOM | 917 | OG1 | THR | H | 107 | 7.652 | −16.005 | 29.783 | 1.00 | 34.12 | O |
| ATOM | 918 | CG2 | THR | H | 107 | 5.372 | −15.263 | 29.783 | 1.00 | 34.17 | C |
| ATOM | 919 | C | THR | H | 107 | 4.512 | −18.108 | 29.714 | 1.00 | 34.60 | C |
| ATOM | 920 | O | THR | H | 107 | 4.225 | −18.347 | 28.524 | 1.00 | 34.60 | O |
| ATOM | 921 | N | MET | H | 108 | 3.637 | −18.253 | 30.710 | 1.00 | 34.34 | N |
| ATOM | 922 | CA | MET | H | 108 | 2.252 | −18.634 | 30.450 | 1.00 | 34.36 | C |
| ATOM | 923 | CB | MET | H | 108 | 1.569 | −19.192 | 31.706 | 1.00 | 34.90 | C |
| ATOM | 924 | CG | MET | H | 108 | 0.175 | −19.829 | 31.417 | 1.00 | 37.49 | C |
| ATOM | 925 | SD | MET | H | 108 | 0.257 | −21.163 | 30.175 | 1.00 | 44.96 | S |
| ATOM | 926 | CE | MET | H | 108 | −1.342 | −21.047 | 29.330 | 1.00 | 42.71 | C |
| ATOM | 927 | C | MET | H | 108 | 1.523 | −17.403 | 29.946 | 1.00 | 34.04 | C |
| ATOM | 928 | O | MET | H | 108 | 1.550 | −16.352 | 30.596 | 1.00 | 33.75 | O |
| ATOM | 929 | N | VAL | H | 109 | 0.908 | −17.518 | 28.769 | 1.00 | 33.35 | N |
| ATOM | 930 | CA | VAL | H | 109 | 0.086 | −16.435 | 28.237 | 1.00 | 32.50 | C |
| ATOM | 931 | CB | VAL | H | 109 | 0.611 | −15.946 | 26.850 | 1.00 | 33.18 | C |
| ATOM | 932 | CG1 | VAL | H | 109 | −0.351 | −14.974 | 26.191 | 1.00 | 31.02 | C |
| ATOM | 933 | CG2 | VAL | H | 109 | 2.004 | −15.331 | 26.964 | 1.00 | 30.70 | C |
| ATOM | 934 | C | VAL | H | 109 | −1.373 | −16.895 | 28.150 | 1.00 | 32.71 | C |
| ATOM | 935 | O | VAL | H | 109 | −1.686 | −17.928 | 27.552 | 1.00 | 32.57 | O |
| ATOM | 936 | N | THR | H | 110 | −2.264 | −16.135 | 28.767 | 1.00 | 33.08 | N |
| ATOM | 937 | CA | THR | H | 110 | −3.687 | −16.449 | 28.730 | 1.00 | 33.33 | C |
| ATOM | 938 | CB | THR | H | 110 | −4.247 | −16.679 | 30.171 | 1.00 | 33.57 | C |
| ATOM | 939 | OG1 | THR | H | 110 | −3.605 | −17.833 | 30.726 | 1.00 | 33.39 | O |
| ATOM | 940 | CG2 | THR | H | 110 | −5.776 | −16.937 | 30.174 | 1.00 | 33.92 | C |
| ATOM | 941 | C | THR | H | 110 | −4.387 | −15.329 | 27.978 | 1.00 | 33.38 | C |
| ATOM | 942 | O | THR | H | 110 | −4.255 | −14.136 | 28.327 | 1.00 | 34.05 | O |
| ATOM | 943 | N | VAL | H | 111 | −5.086 | −15.693 | 26.908 | 1.00 | 32.90 | N |
| ATOM | 944 | CA | VAL | H | 111 | −5.857 | −14.697 | 26.149 | 1.00 | 32.60 | C |
| ATOM | 945 | CB | VAL | H | 111 | −5.439 | −14.614 | 24.656 | 1.00 | 32.65 | C |
| ATOM | 946 | CG1 | VAL | H | 111 | −6.279 | −13.552 | 23.932 | 1.00 | 31.96 | C |
| ATOM | 947 | CG2 | VAL | H | 111 | −3.942 | −14.292 | 24.532 | 1.00 | 31.86 | C |
| ATOM | 948 | C | VAL | H | 111 | −7.329 | −15.038 | 26.266 | 1.00 | 32.72 | C |
| ATOM | 949 | O | VAL | H | 111 | −7.767 | −16.050 | 25.730 | 1.00 | 32.52 | O |
| ATOM | 950 | N | SER | H | 112 | −8.068 | −14.195 | 26.985 | 1.00 | 33.08 | N |
| ATOM | 951 | CA | SER | H | 112 | −9.498 | −14.406 | 27.259 | 1.00 | 33.64 | C |
| ATOM | 952 | CB | SER | H | 112 | −9.675 | −15.287 | 28.489 | 1.00 | 33.58 | C |
| ATOM | 953 | OG | SER | H | 112 | −11.034 | −15.627 | 28.737 | 1.00 | 33.66 | O |
| ATOM | 954 | C | SER | H | 112 | −10.179 | −13.063 | 27.492 | 1.00 | 33.77 | C |
| ATOM | 955 | O | SER | H | 112 | −9.547 | −12.116 | 27.939 | 1.00 | 33.77 | O |
| ATOM | 956 | N | SER | H | 113 | −11.464 | −12.967 | 27.172 | 1.00 | 34.37 | N |
| ATOM | 957 | CA | SER | H | 113 | −12.220 | −11.734 | 27.458 | 1.00 | 34.88 | C |
| ATOM | 958 | CB | SER | H | 113 | −13.390 | −11.555 | 26.478 | 1.00 | 34.91 | C |
| ATOM | 959 | OG | SER | H | 113 | −14.325 | −12.632 | 26.563 | 1.00 | 34.07 | O |
| ATOM | 960 | C | SER | H | 113 | −12.739 | −11.733 | 28.893 | 1.00 | 35.52 | C |
| ATOM | 961 | O | SER | H | 113 | −13.269 | −10.713 | 29.375 | 1.00 | 35.53 | O |
| ATOM | 962 | N | ALA | H | 114 | −12.587 | −12.867 | 29.579 | 1.00 | 35.97 | N |
| ATOM | 963 | CA | ALA | H | 114 | −13.066 | −12.993 | 30.966 | 1.00 | 36.98 | C |
| ATOM | 964 | CB | ALA | H | 114 | −12.985 | −14.401 | 31.436 | 1.00 | 36.36 | C |
| ATOM | 965 | C | ALA | H | 114 | −12.326 | −12.070 | 31.923 | 1.00 | 37.86 | C |
| ATOM | 966 | O | ALA | H | 114 | −11.206 | −11.640 | 31.648 | 1.00 | 38.29 | O |
| ATOM | 967 | N | SER | H | 115 | −12.974 | −11.744 | 33.037 | 1.00 | 39.20 | N |
| ATOM | 968 | CA | SER | H | 115 | −12.378 | −10.869 | 34.047 | 1.00 | 40.18 | C |
| ATOM | 969 | CB | SER | H | 115 | −13.340 | −9.747 | 34.421 | 1.00 | 40.06 | C |
| ATOM | 970 | OG | SER | H | 115 | −13.360 | −8.760 | 33.409 | 1.00 | 42.05 | O |
| ATOM | 971 | C | SER | H | 115 | −11.995 | −11.650 | 35.289 | 1.00 | 40.60 | C |
| ATOM | 972 | O | SER | H | 115 | −12.599 | −12.676 | 35.601 | 1.00 | 40.37 | O |
| ATOM | 973 | N | THR | H | 116 | −10.994 | −11.138 | 35.995 | 1.00 | 41.55 | N |
| ATOM | 974 | CA | THR | H | 116 | −10.529 | −11.710 | 37.250 | 1.00 | 42.44 | C |
| ATOM | 975 | CB | THR | H | 116 | −9.524 | −10.763 | 37.945 | 1.00 | 42.42 | C |
| ATOM | 976 | OG1 | THR | H | 116 | −8.432 | −10.528 | 37.056 | 1.00 | 42.59 | O |
| ATOM | 977 | CG2 | THR | H | 116 | −8.967 | −11.378 | 39.244 | 1.00 | 43.07 | C |
| ATOM | 978 | C | THR | H | 116 | −11.693 | −12.014 | 38.169 | 1.00 | 43.06 | C |
| ATOM | 979 | O | THR | H | 116 | −12.514 | −11.141 | 38.452 | 1.00 | 43.24 | O |
| ATOM | 980 | N | LYS | H | 117 | −11.778 | −13.270 | 38.599 | 1.00 | 43.70 | N |
| ATOM | 981 | CA | LYS | H | 117 | −12.774 | −13.674 | 39.564 | 1.00 | 44.51 | C |
| ATOM | 982 | CB | LYS | H | 117 | −14.036 | −14.213 | 38.884 | 1.00 | 44.97 | C |
| ATOM | 983 | CG | LYS | H | 117 | −15.180 | −14.545 | 39.855 | 1.00 | 45.53 | C |
| ATOM | 984 | CD | LYS | H | 117 | −16.284 | −15.311 | 39.169 | 1.00 | 48.73 | C |
| ATOM | 985 | CE | LYS | H | 117 | −17.338 | −15.819 | 40.159 | 1.00 | 51.25 | C |
| ATOM | 986 | NZ | LYS | H | 117 | −16.926 | −17.061 | 40.934 | 1.00 | 54.53 | N |
| ATOM | 987 | C | LYS | H | 117 | −12.147 | −14.728 | 40.445 | 1.00 | 45.30 | C |
| ATOM | 988 | O | LYS | H | 117 | −11.388 | −15.584 | 39.965 | 1.00 | 45.32 | O |
| ATOM | 989 | N | GLY | H | 118 | −12.447 | −14.638 | 41.740 | 1.00 | 45.89 | N |
| ATOM | 990 | CA | GLY | H | 118 | −11.970 | −15.600 | 42.725 | 1.00 | 46.36 | C |
| ATOM | 991 | C | GLY | H | 118 | −12.834 | −16.842 | 42.695 | 1.00 | 46.46 | C |
| ATOM | 992 | O | GLY | H | 118 | −13.990 | −16.786 | 42.283 | 1.00 | 46.22 | O |
| ATOM | 993 | N | PRO | H | 119 | −12.278 | −17.980 | 43.133 | 1.00 | 46.88 | N |
| ATOM | 994 | CA | PRO | H | 119 | −13.055 | −19.218 | 43.100 | 1.00 | 47.46 | C |
| ATOM | 995 | CB | PRO | H | 119 | −11.996 | −20.297 | 43.309 | 1.00 | 47.35 | C |
| ATOM | 996 | CG | PRO | H | 119 | −10.898 | −19.612 | 44.064 | 1.00 | 47.48 | C |
| ATOM | 997 | CD | PRO | H | 119 | −10.901 | −18.190 | 43.620 | 1.00 | 46.81 | C |
| ATOM | 998 | C | PRO | H | 119 | −14.081 | −19.303 | 44.217 | 1.00 | 47.90 | C |
| ATOM | 999 | O | PRO | H | 119 | −13.887 | −18.721 | 45.287 | 1.00 | 48.16 | O |
| ATOM | 1000 | N | SER | H | 120 | −15.171 | −20.010 | 43.957 | 1.00 | 48.36 | N |
| ATOM | 1001 | CA | SER | H | 120 | −16.022 | −20.486 | 45.030 | 1.00 | 49.55 | C |
| ATOM | 1002 | CB | SER | H | 120 | −17.474 | −20.535 | 44.579 | 1.00 | 49.21 | C |
| ATOM | 1003 | OG | SER | H | 120 | −17.839 | −19.301 | 44.000 | 1.00 | 51.05 | O |
| ATOM | 1004 | C | SER | H | 120 | −15.529 | −21.889 | 45.387 | 1.00 | 49.81 | C |
| ATOM | 1005 | O | SER | H | 120 | −15.356 | −22.731 | 44.491 | 1.00 | 50.20 | O |
| ATOM | 1006 | N | VAL | H | 121 | −15.282 | −22.126 | 46.678 | 1.00 | 49.93 | N |
| ATOM | 1007 | CA | VAL | H | 121 | −14.795 | −23.428 | 47.153 | 1.00 | 49.80 | C |
| ATOM | 1008 | CB | VAL | H | 121 | −13.546 | −23.298 | 48.073 | 1.00 | 49.66 | C |
| ATOM | 1009 | CG1 | VAL | H | 121 | −12.940 | −24.660 | 48.344 | 1.00 | 48.91 | C |
| ATOM | 1010 | CG2 | VAL | H | 121 | −12.501 | −22.384 | 47.449 | 1.00 | 49.09 | C |
| ATOM | 1011 | C | VAL | H | 121 | −15.910 | −24.199 | 47.855 | 1.00 | 49.93 | C |
| ATOM | 1012 | O | VAL | H | 121 | −16.500 | −23.721 | 48.807 | 1.00 | 50.29 | O |
| ATOM | 1013 | N | PHE | H | 122 | −16.204 | −25.394 | 47.368 | 1.00 | 50.10 | N |
| ATOM | 1014 | CA | PHE | H | 122 | −17.292 | −26.187 | 47.918 | 1.00 | 50.38 | C |
| ATOM | 1015 | CB | PHE | H | 122 | −18.420 | −26.341 | 46.896 | 1.00 | 50.03 | C |
| ATOM | 1016 | CG | PHE | H | 122 | −18.983 | −25.032 | 46.407 | 1.00 | 50.62 | C |
| ATOM | 1017 | CD1 | PHE | H | 122 | −18.931 | −24.701 | 45.050 | 1.00 | 50.55 | C |
| ATOM | 1018 | CE1 | PHE | H | 122 | −19.457 | −23.483 | 44.580 | 1.00 | 50.25 | C |
| ATOM | 1019 | CZ | PHE | H | 122 | −20.037 | −22.580 | 45.475 | 1.00 | 49.25 | C |
| ATOM | 1020 | CE2 | PHE | H | 122 | −20.089 | −22.894 | 46.831 | 1.00 | 50.46 | C |
| ATOM | 1021 | CD2 | PHE | H | 122 | −19.562 | −24.123 | 47.295 | 1.00 | 50.21 | C |
| ATOM | 1022 | C | PHE | H | 122 | −16.790 | −27.549 | 48.388 | 1.00 | 50.57 | C |
| ATOM | 1023 | O | PHE | H | 122 | −15.867 | −28.107 | 47.794 | 1.00 | 50.36 | O |
| ATOM | 1024 | N | PRO | H | 123 | −17.387 | −28.084 | 49.467 | 1.00 | 50.95 | N |
| ATOM | 1025 | CA | PRO | H | 123 | −16.929 | −29.372 | 49.983 | 1.00 | 51.25 | C |
| ATOM | 1026 | CB | PRO | H | 123 | −17.554 | −29.426 | 51.390 | 1.00 | 51.25 | C |
| ATOM | 1027 | CG | PRO | H | 123 | −18.763 | −28.569 | 51.295 | 1.00 | 50.68 | C |
| ATOM | 1028 | CD | PRO | H | 123 | −18.366 | −27.446 | 50.372 | 1.00 | 51.03 | C |
| ATOM | 1029 | C | PRO | H | 123 | −17.404 | −30.567 | 49.165 | 1.00 | 51.50 | C |
| ATOM | 1030 | O | PRO | H | 123 | −18.559 | −30.620 | 48.736 | 1.00 | 51.88 | O |
| ATOM | 1031 | N | LEU | H | 124 | −16.503 | −31.515 | 48.955 | 1.00 | 51.93 | N |
| ATOM | 1032 | CA | LEU | H | 124 | −16.869 | −32.833 | 48.480 | 1.00 | 52.75 | C |
| ATOM | 1033 | CB | LEU | H | 124 | −15.918 | −33.299 | 47.373 | 1.00 | 52.29 | C |
| ATOM | 1034 | CG | LEU | H | 124 | −15.961 | −32.479 | 46.075 | 1.00 | 52.79 | C |
| ATOM | 1035 | CD1 | LEU | H | 124 | −14.835 | −32.864 | 45.120 | 1.00 | 52.18 | C |
| ATOM | 1036 | CD2 | LEU | H | 124 | −17.310 | −32.621 | 45.379 | 1.00 | 52.94 | C |
| ATOM | 1037 | C | LEU | H | 124 | −16.836 | −33.749 | 49.702 | 1.00 | 53.76 | C |
| ATOM | 1038 | O | LEU | H | 124 | −15.769 | −34.248 | 50.102 | 1.00 | 53.63 | O |
| ATOM | 1039 | N | ALA | H | 125 | −18.010 | −33.932 | 50.308 | 1.00 | 55.00 | N |
| ATOM | 1040 | CA | ALA | H | 125 | −18.119 | −34.558 | 51.628 | 1.00 | 56.64 | C |
| ATOM | 1041 | CB | ALA | H | 125 | −19.462 | −34.198 | 52.302 | 1.00 | 56.61 | C |
| ATOM | 1042 | C | ALA | H | 125 | −17.950 | −36.069 | 51.573 | 1.00 | 57.74 | C |
| ATOM | 1043 | O | ALA | H | 125 | −18.484 | −36.723 | 50.674 | 1.00 | 57.53 | O |
| ATOM | 1044 | N | PRO | H | 126 | −17.206 | −36.630 | 52.546 | 1.00 | 59.13 | N |
| ATOM | 1045 | CA | PRO | H | 126 | −17.157 | −38.091 | 52.666 | 1.00 | 60.02 | C |
| ATOM | 1046 | CB | PRO | H | 126 | −16.077 | −38.326 | 53.733 | 1.00 | 59.93 | C |
| ATOM | 1047 | CG | PRO | H | 126 | −16.023 | −37.056 | 54.526 | 1.00 | 59.69 | C |
| ATOM | 1048 | CD | PRO | H | 126 | −16.373 | −35.951 | 53.561 | 1.00 | 59.26 | C |
| ATOM | 1049 | C | PRO | H | 126 | −18.521 | −38.611 | 53.119 | 1.00 | 60.94 | C |
| ATOM | 1050 | O | PRO | H | 126 | −19.032 | −38.192 | 54.163 | 1.00 | 61.38 | O |
| ATOM | 1051 | N | SER | H | 127 | −19.121 | −39.489 | 52.321 | 1.00 | 62.18 | N |
| ATOM | 1052 | CA | SER | H | 127 | −20.443 | −40.032 | 52.646 | 1.00 | 63.10 | C |
| ATOM | 1053 | CB | SER | H | 127 | −21.204 | −40.437 | 51.372 | 1.00 | 63.00 | C |
| ATOM | 1054 | OG | SER | H | 127 | −20.715 | −41.653 | 50.831 | 1.00 | 63.54 | O |
| ATOM | 1055 | C | SER | H | 127 | −20.336 | −41.194 | 53.646 | 1.00 | 63.49 | C |
| ATOM | 1056 | O | SER | H | 127 | −19.240 | −41.515 | 54.117 | 1.00 | 63.49 | O |
| ATOM | 1057 | N | SER | H | 128 | −21.472 | −41.808 | 53.972 | 1.00 | 63.95 | N |
| ATOM | 1058 | CA | SER | H | 128 | −21.506 | −42.922 | 54.925 | 1.00 | 64.33 | C |
| ATOM | 1059 | CB | SER | H | 128 | −22.148 | −42.470 | 56.241 | 1.00 | 64.44 | C |
| ATOM | 1060 | OG | SER | H | 128 | −23.323 | −41.708 | 55.997 | 1.00 | 64.12 | O |
| ATOM | 1061 | C | SER | H | 128 | −22.245 | −44.136 | 54.359 | 1.00 | 64.36 | C |
| ATOM | 1062 | O | SER | H | 128 | −21.638 | −45.014 | 53.740 | 1.00 | 64.42 | O |
| ATOM | 1063 | N | GLY | H | 133 | −16.036 | −49.054 | 53.342 | 1.00 | 57.57 | N |
| ATOM | 1064 | CA | GLY | H | 133 | −15.239 | −49.934 | 54.199 | 1.00 | 57.22 | C |
| ATOM | 1065 | C | GLY | H | 133 | −13.986 | −49.259 | 54.734 | 1.00 | 56.93 | C |
| ATOM | 1066 | O | GLY | H | 133 | −14.070 | −48.264 | 55.459 | 1.00 | 56.85 | O |
| ATOM | 1067 | N | GLY | H | 134 | −12.825 | −49.797 | 54.354 | 1.00 | 56.39 | N |
| ATOM | 1068 | CA | GLY | H | 134 | −11.524 | −49.341 | 54.860 | 1.00 | 55.30 | C |
| ATOM | 1069 | C | GLY | H | 134 | −11.095 | −47.917 | 54.515 | 1.00 | 54.70 | C |
| ATOM | 1070 | O | GLY | H | 134 | −10.486 | −47.232 | 55.348 | 1.00 | 54.25 | O |
| ATOM | 1071 | N | THR | H | 135 | −11.412 | −47.466 | 53.297 | 1.00 | 53.98 | N |
| ATOM | 1072 | CA | THR | H | 135 | −10.937 | −46.166 | 52.808 | 1.00 | 53.42 | C |
| ATOM | 1073 | CB | THR | H | 135 | −9.957 | −46.321 | 51.630 | 1.00 | 53.65 | C |
| ATOM | 1074 | OG1 | THR | H | 135 | −10.551 | −47.185 | 50.658 | 1.00 | 55.85 | O |
| ATOM | 1075 | CG2 | THR | H | 135 | −8.624 | −46.912 | 52.088 | 1.00 | 53.64 | C |
| ATOM | 1076 | C | THR | H | 135 | −12.059 | −45.228 | 52.376 | 1.00 | 52.32 | C |
| ATOM | 1077 | O | THR | H | 135 | −13.017 | −45.638 | 51.716 | 1.00 | 51.64 | O |
| ATOM | 1078 | N | ALA | H | 136 | −11.908 | −43.959 | 52.752 | 1.00 | 51.19 | N |
| ATOM | 1079 | CA | ALA | H | 136 | −12.855 | −42.905 | 52.394 | 1.00 | 50.12 | C |
| ATOM | 1080 | CB | ALA | H | 136 | −13.390 | −42.231 | 53.651 | 1.00 | 49.96 | C |
| ATOM | 1081 | C | ALA | H | 136 | −12.176 | −41.871 | 51.504 | 1.00 | 49.51 | C |
| ATOM | 1082 | O | ALA | H | 136 | −10.966 | −41.647 | 51.620 | 1.00 | 49.07 | O |
| ATOM | 1083 | N | ALA | H | 137 | −12.964 | −41.248 | 50.625 | 1.00 | 48.58 | N |
| ATOM | 1084 | CA | ALA | H | 137 | −12.503 | −40.135 | 49.795 | 1.00 | 47.83 | C |
| ATOM | 1085 | CB | ALA | H | 137 | −12.551 | −40.501 | 48.315 | 1.00 | 47.97 | C |
| ATOM | 1086 | C | ALA | H | 137 | −13.314 | −38.876 | 50.045 | 1.00 | 47.04 | C |
| ATOM | 1087 | O | ALA | H | 137 | −14.544 | −38.922 | 50.166 | 1.00 | 47.09 | O |
| ATOM | 1088 | N | LEU | H | 138 | −12.605 | −37.755 | 50.107 | 1.00 | 46.11 | N |
| ATOM | 1089 | CA | LEU | H | 138 | −13.200 | −36.436 | 50.299 | 1.00 | 45.79 | C |
| ATOM | 1090 | CB | LEU | H | 138 | −13.180 | −36.062 | 51.784 | 1.00 | 45.56 | C |
| ATOM | 1091 | CG | LEU | H | 138 | −11.810 | −36.084 | 52.458 | 1.00 | 45.69 | C |
| ATOM | 1092 | CD1 | LEU | H | 138 | −11.297 | −34.678 | 52.734 | 1.00 | 46.09 | C |
| ATOM | 1093 | CD2 | LEU | H | 138 | −11.889 | −36.884 | 53.755 | 1.00 | 45.96 | C |
| ATOM | 1094 | C | LEU | H | 138 | −12.431 | −35.404 | 49.470 | 1.00 | 45.56 | C |
| ATOM | 1095 | O | LEU | H | 138 | −11.351 | −35.703 | 48.943 | 1.00 | 45.39 | O |
| ATOM | 1096 | N | GLY | H | 139 | −12.976 | −34.195 | 49.350 | 1.00 | 45.37 | N |
| ATOM | 1097 | CA | GLY | H | 139 | −12.305 | −33.163 | 48.572 | 1.00 | 45.46 | C |
| ATOM | 1098 | C | GLY | H | 139 | −12.933 | −31.786 | 48.565 | 1.00 | 45.66 | C |
| ATOM | 1099 | O | GLY | H | 139 | −13.844 | −31.490 | 49.350 | 1.00 | 45.36 | O |
| ATOM | 1100 | N | CYS | H | 140 | −12.423 | −30.940 | 47.672 | 1.00 | 45.27 | N |
| ATOM | 1101 | CA | CYS | H | 140 | −12.934 | −29.589 | 47.491 | 1.00 | 45.35 | C |
| ATOM | 1102 | CB | CYS | H | 140 | −11.994 | −28.566 | 48.131 | 1.00 | 45.26 | C |
| ATOM | 1103 | SG | CYS | H | 140 | −12.118 | −28.468 | 49.955 | 1.00 | 48.58 | S |
| ATOM | 1104 | C | CYS | H | 140 | −13.163 | −29.265 | 46.006 | 1.00 | 44.65 | C |
| ATOM | 1105 | O | CYS | H | 140 | −12.279 | −29.462 | 45.172 | 1.00 | 44.27 | O |
| ATOM | 1106 | N | LEU | H | 141 | −14.362 | −28.783 | 45.704 | 1.00 | 44.12 | N |
| ATOM | 1107 | CA | LEU | H | 141 | −14.702 | −28.264 | 44.386 | 1.00 | 44.32 | C |
| ATOM | 1108 | CB | LEU | H | 141 | −16.197 | −28.479 | 44.097 | 1.00 | 44.27 | C |
| ATOM | 1109 | CG | LEU | H | 141 | −16.799 | −28.018 | 42.763 | 1.00 | 44.05 | C |
| ATOM | 1110 | CD1 | LEU | H | 141 | −16.016 | −28.529 | 41.545 | 1.00 | 44.88 | C |
| ATOM | 1111 | CD2 | LEU | H | 141 | −18.225 | −28.471 | 42.685 | 1.00 | 42.18 | C |
| ATOM | 1112 | C | LEU | H | 141 | −14.333 | −26.785 | 44.307 | 1.00 | 44.01 | C |
| ATOM | 1113 | O | LEU | H | 141 | −14.869 | −25.958 | 45.051 | 1.00 | 44.05 | O |
| ATOM | 1114 | N | VAL | H | 142 | −13.397 | −26.464 | 43.420 | 1.00 | 43.67 | N |
| ATOM | 1115 | CA | VAL | H | 142 | −12.940 | −25.094 | 43.218 | 1.00 | 43.53 | C |
| ATOM | 1116 | CB | VAL | H | 142 | −11.396 | −25.011 | 43.251 | 1.00 | 43.63 | C |
| ATOM | 1117 | CG1 | VAL | H | 142 | −10.936 | −23.567 | 43.320 | 1.00 | 44.11 | C |
| ATOM | 1118 | CG2 | VAL | H | 142 | −10.833 | −25.784 | 44.449 | 1.00 | 43.82 | C |
| ATOM | 1119 | C | VAL | H | 142 | −13.501 | −24.605 | 41.868 | 1.00 | 43.66 | C |
| ATOM | 1120 | O | VAL | H | 142 | −13.007 | −24.997 | 40.797 | 1.00 | 43.73 | O |
| ATOM | 1121 | N | LYS | H | 143 | −14.534 | −23.759 | 41.935 | 1.00 | 43.23 | N |
| ATOM | 1122 | CA | LYS | H | 143 | −15.357 | −23.410 | 40.770 | 1.00 | 42.72 | C |
| ATOM | 1123 | CB | LYS | H | 143 | −16.808 | −23.863 | 41.007 | 1.00 | 42.75 | C |
| ATOM | 1124 | CG | LYS | H | 143 | −17.533 | −24.198 | 39.733 | 1.00 | 42.96 | C |
| ATOM | 1125 | CD | LYS | H | 143 | −19.015 | −24.349 | 39.913 | 1.00 | 43.80 | C |
| ATOM | 1126 | CE | LYS | H | 143 | −19.631 | −24.680 | 38.580 | 1.00 | 44.54 | C |
| ATOM | 1127 | NZ | LYS | H | 143 | −21.089 | −24.534 | 38.591 | 1.00 | 47.76 | N |
| ATOM | 1128 | C | LYS | H | 143 | −15.342 | −21.922 | 40.364 | 1.00 | 42.21 | C |
| ATOM | 1129 | O | LYS | H | 143 | −15.310 | −21.027 | 41.215 | 1.00 | 41.87 | O |
| ATOM | 1130 | N | ASP | H | 144 | −15.365 | −21.679 | 39.054 | 1.00 | 41.51 | N |
| ATOM | 1131 | CA | ASP | H | 144 | −15.622 | −20.341 | 38.491 | 1.00 | 40.94 | C |
| ATOM | 1132 | CB | ASP | H | 144 | −16.993 | −19.806 | 38.919 | 1.00 | 41.36 | C |
| ATOM | 1133 | CG | ASP | H | 144 | −18.140 | −20.653 | 38.419 | 1.00 | 42.24 | C |
| ATOM | 1134 | OD1 | ASP | H | 144 | −17.953 | −21.508 | 37.531 | 1.00 | 43.56 | O |
| ATOM | 1135 | OD2 | ASP | H | 144 | −19.253 | −20.447 | 38.916 | 1.00 | 43.96 | O |
| ATOM | 1136 | C | ASP | H | 144 | −14.576 | −19.303 | 38.833 | 1.00 | 40.07 | C |
| ATOM | 1137 | O | ASP | H | 144 | −14.902 | −18.229 | 39.317 | 1.00 | 40.56 | O |
| ATOM | 1138 | N | TYR | H | 145 | −13.318 | −19.601 | 38.564 | 1.00 | 38.77 | N |
| ATOM | 1139 | CA | TYR | H | 145 | −12.272 | −18.617 | 38.791 | 1.00 | 37.85 | C |
| ATOM | 1140 | CB | TYR | H | 145 | −11.251 | −19.133 | 39.809 | 1.00 | 37.00 | C |
| ATOM | 1141 | CG | TYR | H | 145 | −10.456 | −20.344 | 39.349 | 1.00 | 35.24 | C |
| ATOM | 1142 | CD1 | TYR | H | 145 | −10.941 | −21.631 | 39.545 | 1.00 | 32.82 | C |
| ATOM | 1143 | CE1 | TYR | H | 145 | −10.206 | −22.749 | 39.142 | 1.00 | 33.25 | C |
| ATOM | 1144 | CZ | TYR | H | 145 | −8.973 | −22.569 | 38.546 | 1.00 | 33.51 | C |
| ATOM | 1145 | OH | TYR | H | 145 | −8.242 | −23.665 | 38.138 | 1.00 | 35.94 | O |
| ATOM | 1146 | CE2 | TYR | H | 145 | −8.469 | −21.299 | 38.344 | 1.00 | 32.84 | C |
| ATOM | 1147 | CD2 | TYR | H | 145 | −9.207 | −20.195 | 38.752 | 1.00 | 33.07 | C |
| ATOM | 1148 | C | TYR | H | 145 | −11.567 | −18.260 | 37.500 | 1.00 | 37.80 | C |
| ATOM | 1149 | O | TYR | H | 145 | −11.580 | −19.034 | 36.529 | 1.00 | 37.27 | O |
| ATOM | 1150 | N | PHE | H | 146 | −10.936 | −17.089 | 37.508 | 1.00 | 37.81 | N |
| ATOM | 1151 | CA | PHE | H | 146 | −10.142 | −16.647 | 36.373 | 1.00 | 37.92 | C |
| ATOM | 1152 | CB | PHE | H | 146 | −11.034 | −16.036 | 35.260 | 1.00 | 37.53 | C |
| ATOM | 1153 | CG | PHE | H | 146 | −10.259 | −15.583 | 34.050 | 1.00 | 35.71 | C |
| ATOM | 1154 | CD1 | PHE | H | 146 | −9.754 | −14.295 | 33.973 | 1.00 | 34.67 | C |
| ATOM | 1155 | CE1 | PHE | H | 146 | −9.005 | −13.880 | 32.859 | 1.00 | 33.86 | C |
| ATOM | 1156 | CZ | PHE | H | 146 | −8.764 | −14.766 | 31.830 | 1.00 | 33.72 | C |
| ATOM | 1157 | CE2 | PHE | H | 146 | −9.261 | −16.049 | 31.895 | 1.00 | 33.02 | C |
| ATOM | 1158 | CD2 | PHE | H | 146 | −10.000 | −16.462 | 33.005 | 1.00 | 35.02 | C |
| ATOM | 1159 | C | PHE | H | 146 | −9.093 | −15.647 | 36.830 | 1.00 | 38.09 | C |
| ATOM | 1160 | O | PHE | H | 146 | −9.389 | −14.763 | 37.635 | 1.00 | 38.72 | O |
| ATOM | 1161 | N | PRO | H | 147 | −7.863 | −15.768 | 36.313 | 1.00 | 38.55 | N |
| ATOM | 1162 | CA | PRO | H | 147 | −7.410 | −16.814 | 35.418 | 1.00 | 38.93 | C |
| ATOM | 1163 | CB | PRO | H | 147 | −6.316 | −16.111 | 34.627 | 1.00 | 38.93 | C |
| ATOM | 1164 | CG | PRO | H | 147 | −5.663 | −15.221 | 35.647 | 1.00 | 38.57 | C |
| ATOM | 1165 | CD | PRO | H | 147 | −6.803 | −14.764 | 36.545 | 1.00 | 39.19 | C |
| ATOM | 1166 | C | PRO | H | 147 | −6.812 | −17.968 | 36.219 | 1.00 | 39.78 | C |
| ATOM | 1167 | O | PRO | H | 147 | −7.019 | −18.054 | 37.440 | 1.00 | 39.52 | O |
| ATOM | 1168 | N | GLU | H | 148 | −6.107 | −18.854 | 35.525 | 1.00 | 40.47 | N |
| ATOM | 1169 | CA | GLU | H | 148 | −5.162 | −19.754 | 36.164 | 1.00 | 41.90 | C |
| ATOM | 1170 | CB | GLU | H | 148 | −4.700 | −20.827 | 35.175 | 1.00 | 41.56 | C |
| ATOM | 1171 | CG | GLU | H | 148 | −5.743 | −21.886 | 34.920 | 1.00 | 43.47 | C |
| ATOM | 1172 | CD | GLU | H | 148 | −5.621 | −23.058 | 35.859 | 1.00 | 45.70 | C |
| ATOM | 1173 | OE1 | GLU | H | 148 | −5.950 | −22.914 | 37.068 | 1.00 | 46.77 | O |
| ATOM | 1174 | OE2 | GLU | H | 148 | −5.196 | −24.134 | 35.379 | 1.00 | 44.53 | O |
| ATOM | 1175 | C | GLU | H | 148 | −3.971 | −18.916 | 36.660 | 1.00 | 42.39 | C |
| ATOM | 1176 | O | GLU | H | 148 | −3.637 | −17.896 | 36.050 | 1.00 | 42.61 | O |
| ATOM | 1177 | N | PRO | H | 149 | −3.295 | −19.367 | 37.738 | 1.00 | 43.04 | N |
| ATOM | 1178 | CA | PRO | H | 149 | −3.532 | −20.653 | 38.365 | 1.00 | 43.12 | C |
| ATOM | 1179 | CB | PRO | H | 149 | −2.113 | −21.204 | 38.508 | 1.00 | 43.63 | C |
| ATOM | 1180 | CG | PRO | H | 149 | −1.280 | −19.949 | 38.817 | 1.00 | 43.57 | C |
| ATOM | 1181 | CD | PRO | H | 149 | −2.051 | −18.751 | 38.245 | 1.00 | 43.04 | C |
| ATOM | 1182 | C | PRO | H | 149 | −4.171 | −20.548 | 39.742 | 1.00 | 43.53 | C |
| ATOM | 1183 | O | PRO | H | 149 | −4.222 | −19.463 | 40.351 | 1.00 | 43.62 | O |
| ATOM | 1184 | N | VAL | H | 150 | −4.559 | −21.557 | 40.306 | 1.00 | 43.37 | N |
| ATOM | 1185 | CA | VAL | H | 150 | −5.004 | −21.631 | 41.676 | 1.00 | 43.48 | C |
| ATOM | 1186 | CB | VAL | H | 150 | −6.496 | −22.047 | 41.729 | 1.00 | 43.45 | C |
| ATOM | 1187 | CG1 | VAL | H | 150 | −6.715 | −23.443 | 41.150 | 1.00 | 44.11 | C |
| ATOM | 1188 | CG2 | VAL | H | 150 | −7.039 | −21.930 | 43.149 | 1.00 | 42.28 | C |
| ATOM | 1189 | C | VAL | H | 150 | −4.021 | −22.563 | 42.430 | 1.00 | 43.56 | C |
| ATOM | 1190 | O | VAL | H | 150 | −3.396 | −23.429 | 41.815 | 1.00 | 43.59 | O |
| ATOM | 1191 | N | THR | H | 151 | −3.734 | −22.618 | 43.560 | 1.00 | 43.56 | N |
| ATOM | 1192 | CA | THR | H | 151 | −2.957 | −23.631 | 44.312 | 1.00 | 43.74 | C |
| ATOM | 1193 | CB | THR | H | 151 | −1.615 | −23.096 | 44.906 | 1.00 | 43.77 | C |
| ATOM | 1194 | OG1 | THR | H | 151 | −1.878 | −22.013 | 45.809 | 1.00 | 44.29 | O |
| ATOM | 1195 | CG2 | THR | H | 151 | −0.665 | −22.627 | 43.805 | 1.00 | 43.73 | C |
| ATOM | 1196 | C | THR | H | 151 | −3.796 | −24.228 | 45.442 | 1.00 | 43.52 | C |
| ATOM | 1197 | O | THR | H | 151 | −4.510 | −23.518 | 46.152 | 1.00 | 43.65 | O |
| ATOM | 1198 | N | VAL | H | 152 | −3.716 | −25.541 | 45.581 | 1.00 | 43.56 | N |
| ATOM | 1199 | CA | VAL | H | 152 | −4.457 | −26.257 | 46.609 | 1.00 | 44.16 | C |
| ATOM | 1200 | CB | VAL | H | 152 | −5.588 | −27.151 | 46.025 | 1.00 | 44.13 | C |
| ATOM | 1201 | CG1 | VAL | H | 152 | −6.575 | −27.555 | 47.125 | 1.00 | 43.19 | C |
| ATOM | 1202 | CG2 | VAL | H | 152 | −6.310 | −26.441 | 44.883 | 1.00 | 43.72 | C |
| ATOM | 1203 | C | VAL | H | 152 | −3.501 | −27.131 | 47.413 | 1.00 | 44.48 | C |
| ATOM | 1204 | O | VAL | H | 152 | −2.717 | −27.905 | 46.858 | 1.00 | 43.63 | O |
| ATOM | 1205 | N | SER | H | 153 | −3.556 | −26.969 | 48.727 | 1.00 | 45.31 | N |
| ATOM | 1206 | CA | SER | H | 153 | −2.920 | −27.911 | 49.620 | 1.00 | 46.18 | C |
| ATOM | 1207 | CB | SER | H | 153 | −1.709 | −27.282 | 50.319 | 1.00 | 46.33 | C |
| ATOM | 1208 | OG | SER | H | 153 | −2.075 | −26.117 | 51.033 | 1.00 | 47.86 | O |
| ATOM | 1209 | C | SER | H | 153 | −3.964 | −28.408 | 50.614 | 1.00 | 46.59 | C |
| ATOM | 1210 | O | SER | H | 153 | −5.088 | −27.890 | 50.673 | 1.00 | 46.04 | O |
| ATOM | 1211 | N | TRP | H | 154 | −3.597 | −29.439 | 51.365 | 1.00 | 46.98 | N |
| ATOM | 1212 | CA | TRP | H | 154 | −4.455 | −29.964 | 52.398 | 1.00 | 47.46 | C |
| ATOM | 1213 | CB | TRP | H | 154 | −4.782 | −31.418 | 52.100 | 1.00 | 47.54 | C |
| ATOM | 1214 | CG | TRP | H | 154 | −5.759 | −31.551 | 50.971 | 1.00 | 47.76 | C |
| ATOM | 1215 | CD1 | TRP | H | 154 | −5.472 | −31.684 | 49.637 | 1.00 | 47.03 | C |
| ATOM | 1216 | NE1 | TRP | H | 154 | −6.636 | −31.771 | 48.908 | 1.00 | 46.83 | N |
| ATOM | 1217 | CE2 | TRP | H | 154 | −7.703 | −31.697 | 49.763 | 1.00 | 47.21 | C |
| ATOM | 1218 | CD2 | TRP | H | 154 | −7.186 | −31.557 | 51.076 | 1.00 | 47.61 | C |
| ATOM | 1219 | CE3 | TRP | H | 154 | −8.083 | −31.457 | 52.152 | 1.00 | 46.81 | C |
| ATOM | 1220 | CZ3 | TRP | H | 154 | −9.443 | −31.506 | 51.889 | 1.00 | 46.81 | C |
| ATOM | 1221 | CH2 | TRP | H | 154 | −9.927 | −31.651 | 50.571 | 1.00 | 46.48 | C |
| ATOM | 1222 | CZ2 | TRP | H | 154 | −9.074 | −31.750 | 49.501 | 1.00 | 46.80 | C |
| ATOM | 1223 | C | TRP | H | 154 | −3.762 | −29.810 | 53.741 | 1.00 | 48.11 | C |
| ATOM | 1224 | O | TRP | H | 154 | −2.571 | −30.116 | 53.866 | 1.00 | 47.84 | O |
| ATOM | 1225 | N | ASN | H | 155 | −4.511 | −29.302 | 54.725 | 1.00 | 48.92 | N |
| ATOM | 1226 | CA | ASN | H | 155 | −4.008 | −29.059 | 56.083 | 1.00 | 49.47 | C |
| ATOM | 1227 | CB | ASN | H | 155 | −3.965 | −30.365 | 56.889 | 1.00 | 49.56 | C |
| ATOM | 1228 | CG | ASN | H | 155 | −5.334 | −30.977 | 57.082 | 1.00 | 49.31 | C |
| ATOM | 1229 | OD1 | ASN | H | 155 | −6.352 | −30.327 | 56.870 | 1.00 | 49.07 | O |
| ATOM | 1230 | ND2 | ASN | H | 155 | −5.363 | −32.242 | 57.470 | 1.00 | 48.28 | N |
| ATOM | 1231 | C | ASN | H | 155 | −2.649 | −28.382 | 56.077 | 1.00 | 49.88 | C |
| ATOM | 1232 | O | ASN | H | 155 | −1.674 | −28.914 | 56.608 | 1.00 | 49.94 | O |
| ATOM | 1233 | N | SER | H | 156 | −2.603 | −27.219 | 55.429 | 1.00 | 50.67 | N |
| ATOM | 1234 | CA | SER | H | 156 | −1.404 | −26.382 | 55.298 | 1.00 | 50.89 | C |
| ATOM | 1235 | CB | SER | H | 156 | −1.124 | −25.626 | 56.606 | 1.00 | 51.24 | C |
| ATOM | 1236 | OG | SER | H | 156 | −2.156 | −24.700 | 56.874 | 1.00 | 50.89 | O |
| ATOM | 1237 | C | SER | H | 156 | −0.142 | −27.091 | 54.814 | 1.00 | 51.30 | C |
| ATOM | 1238 | O | SER | H | 156 | 0.966 | −26.643 | 55.104 | 1.00 | 51.67 | O |
| ATOM | 1239 | N | GLY | H | 157 | −0.304 | −28.174 | 54.058 | 1.00 | 51.56 | N |
| ATOM | 1240 | CA | GLY | H | 157 | 0.844 | −28.937 | 53.556 | 1.00 | 51.34 | C |
| ATOM | 1241 | C | GLY | H | 157 | 1.164 | −30.184 | 54.372 | 1.00 | 51.62 | C |
| ATOM | 1242 | O | GLY | H | 157 | 1.901 | −31.071 | 53.909 | 1.00 | 51.37 | O |
| ATOM | 1243 | N | ALA | H | 158 | 0.602 | −30.256 | 55.580 | 1.00 | 51.21 | N |
| ATOM | 1244 | CA | ALA | H | 158 | 0.777 | −31.414 | 56.462 | 1.00 | 51.13 | C |
| ATOM | 1245 | CB | ALA | H | 158 | 0.125 | −31.155 | 57.836 | 1.00 | 50.93 | C |
| ATOM | 1246 | C | ALA | H | 158 | 0.232 | −32.701 | 55.841 | 1.00 | 50.86 | C |
| ATOM | 1247 | O | ALA | H | 158 | 0.737 | −33.783 | 56.118 | 1.00 | 51.19 | O |
| ATOM | 1248 | N | LEU | H | 159 | −0.802 | −32.587 | 55.012 | 1.00 | 50.50 | N |
| ATOM | 1249 | CA | LEU | H | 159 | −1.339 | −33.764 | 54.316 | 1.00 | 50.41 | C |
| ATOM | 1250 | CB | LEU | H | 159 | −2.844 | −33.955 | 54.606 | 1.00 | 50.01 | C |
| ATOM | 1251 | CG | LEU | H | 159 | −3.552 | −35.070 | 53.826 | 1.00 | 49.41 | C |
| ATOM | 1252 | CD1 | LEU | H | 159 | −2.977 | −36.442 | 54.134 | 1.00 | 49.40 | C |
| ATOM | 1253 | CD2 | LEU | H | 159 | −5.027 | −35.041 | 54.116 | 1.00 | 49.69 | C |
| ATOM | 1254 | C | LEU | H | 159 | −1.043 | −33.787 | 52.800 | 1.00 | 50.26 | C |
| ATOM | 1255 | O | LEU | H | 159 | −1.512 | −32.917 | 52.045 | 1.00 | 50.25 | O |
| ATOM | 1256 | N | THR | H | 160 | −0.272 | −34.793 | 52.375 | 1.00 | 49.76 | N |
| ATOM | 1257 | CA | THR | H | 160 | 0.111 | −34.951 | 50.971 | 1.00 | 49.53 | C |
| ATOM | 1258 | CB | THR | H | 160 | 1.599 | −34.578 | 50.703 | 1.00 | 49.70 | C |
| ATOM | 1259 | OG1 | THR | H | 160 | 2.439 | −35.161 | 51.705 | 1.00 | 50.42 | O |
| ATOM | 1260 | CG2 | THR | H | 160 | 1.800 | −33.066 | 50.684 | 1.00 | 49.85 | C |
| ATOM | 1261 | C | THR | H | 160 | −0.143 | −36.346 | 50.403 | 1.00 | 48.96 | C |
| ATOM | 1262 | O | THR | H | 160 | −0.322 | −36.495 | 49.196 | 1.00 | 49.43 | O |
| ATOM | 1263 | N | SER | H | 161 | −0.149 | −37.370 | 51.249 | 1.00 | 48.13 | N |
| ATOM | 1264 | CA | SER | H | 161 | −0.369 | −38.730 | 50.756 | 1.00 | 47.52 | C |
| ATOM | 1265 | CB | SER | H | 161 | −0.043 | −39.770 | 51.830 | 1.00 | 47.39 | C |
| ATOM | 1266 | OG | SER | H | 161 | −0.339 | −41.081 | 51.378 | 1.00 | 46.50 | O |
| ATOM | 1267 | C | SER | H | 161 | −1.797 | −38.899 | 50.233 | 1.00 | 47.26 | C |
| ATOM | 1268 | O | SER | H | 161 | −2.747 | −38.350 | 50.802 | 1.00 | 47.27 | O |
| ATOM | 1269 | N | GLY | H | 162 | −1.932 | −39.628 | 49.126 | 1.00 | 46.84 | N |
| ATOM | 1270 | CA | GLY | H | 162 | −3.246 | −39.909 | 48.526 | 1.00 | 46.55 | C |
| ATOM | 1271 | C | GLY | H | 162 | −3.971 | −38.711 | 47.910 | 1.00 | 46.03 | C |
| ATOM | 1272 | O | GLY | H | 162 | −5.143 | −38.818 | 47.535 | 1.00 | 46.68 | O |
| ATOM | 1273 | N | VAL | H | 163 | −3.290 | −37.573 | 47.813 | 1.00 | 45.16 | N |
| ATOM | 1274 | CA | VAL | H | 163 | −3.893 | −36.355 | 47.254 | 1.00 | 44.84 | C |
| ATOM | 1275 | CB | VAL | H | 163 | −3.204 | −35.052 | 47.786 | 1.00 | 44.43 | C |
| ATOM | 1276 | CG1 | VAL | H | 163 | −3.622 | −33.831 | 46.990 | 1.00 | 44.12 | C |
| ATOM | 1277 | CG2 | VAL | H | 163 | −3.553 | −34.836 | 49.235 | 1.00 | 44.84 | C |
| ATOM | 1278 | C | VAL | H | 163 | −3.909 | −36.359 | 45.720 | 1.00 | 44.20 | C |
| ATOM | 1279 | O | VAL | H | 163 | −2.884 | −36.567 | 45.077 | 1.00 | 44.09 | O |
| ATOM | 1280 | N | HIS | H | 164 | −5.082 | −36.115 | 45.148 | 1.00 | 43.98 | N |
| ATOM | 1281 | CA | HIS | H | 164 | −5.203 | −35.860 | 43.715 | 1.00 | 43.52 | C |
| ATOM | 1282 | CB | HIS | H | 164 | −5.949 | −36.993 | 43.011 | 1.00 | 43.33 | C |
| ATOM | 1283 | CG | HIS | H | 164 | −5.191 | −38.284 | 42.982 | 1.00 | 44.01 | C |
| ATOM | 1284 | ND1 | HIS | H | 164 | −3.898 | −38.377 | 42.513 | 1.00 | 43.37 | N |
| ATOM | 1285 | CE1 | HIS | H | 164 | −3.489 | −39.631 | 42.603 | 1.00 | 44.38 | C |
| ATOM | 1286 | NE2 | HIS | H | 164 | −4.472 | −40.355 | 43.109 | 1.00 | 44.15 | N |
| ATOM | 1287 | CD2 | HIS | H | 164 | −5.551 | −39.538 | 43.348 | 1.00 | 43.16 | C |
| ATOM | 1288 | C | HIS | H | 164 | −5.890 | −34.521 | 43.457 | 1.00 | 43.40 | C |
| ATOM | 1289 | O | HIS | H | 164 | −7.081 | −34.362 | 43.710 | 1.00 | 43.00 | O |
| ATOM | 1290 | N | THR | H | 165 | −5.115 | −33.564 | 42.956 | 1.00 | 43.17 | N |
| ATOM | 1291 | CA | THR | H | 165 | −5.640 | −32.280 | 42.501 | 1.00 | 42.96 | C |
| ATOM | 1292 | CB | THR | H | 165 | −4.737 | −31.128 | 42.945 | 1.00 | 43.06 | C |
| ATOM | 1293 | OG1 | THR | H | 165 | −4.665 | −31.130 | 44.373 | 1.00 | 44.11 | O |
| ATOM | 1294 | CG2 | THR | H | 165 | −5.274 | −29.763 | 42.475 | 1.00 | 43.10 | C |
| ATOM | 1295 | C | THR | H | 165 | −5.729 | −32.331 | 40.979 | 1.00 | 42.56 | C |
| ATOM | 1296 | O | THR | H | 165 | −4.708 | −32.408 | 40.282 | 1.00 | 42.49 | O |
| ATOM | 1297 | N | PHE | H | 166 | −6.952 | −32.316 | 40.472 | 1.00 | 41.59 | N |
| ATOM | 1298 | CA | PHE | H | 166 | −7.161 | −32.401 | 39.026 | 1.00 | 41.14 | C |
| ATOM | 1299 | CB | PHE | H | 166 | −8.621 | −32.730 | 38.739 | 1.00 | 41.02 | C |
| ATOM | 1300 | CG | PHE | H | 166 | −8.990 | −34.113 | 39.154 | 1.00 | 40.64 | C |
| ATOM | 1301 | CD1 | PHE | H | 166 | −8.739 | −35.190 | 38.313 | 1.00 | 40.44 | C |
| ATOM | 1302 | CE1 | PHE | H | 166 | −9.052 | −36.472 | 38.688 | 1.00 | 40.26 | C |
| ATOM | 1303 | CZ | PHE | H | 166 | −9.608 | −36.714 | 39.936 | 1.00 | 40.07 | C |
| ATOM | 1304 | CE2 | PHE | H | 166 | −9.860 | −35.655 | 40.792 | 1.00 | 40.78 | C |
| ATOM | 1305 | CD2 | PHE | H | 166 | −9.540 | −34.356 | 40.404 | 1.00 | 41.40 | C |
| ATOM | 1306 | C | PHE | H | 166 | −6.698 | −31.170 | 38.251 | 1.00 | 40.25 | C |
| ATOM | 1307 | O | PHE | H | 166 | −6.637 | −30.075 | 38.805 | 1.00 | 40.05 | O |
| ATOM | 1308 | N | PRO | H | 167 | −6.328 | −31.360 | 36.974 | 1.00 | 40.10 | N |
| ATOM | 1309 | CA | PRO | H | 167 | −6.102 | −30.238 | 36.068 | 1.00 | 39.70 | C |
| ATOM | 1310 | CB | PRO | H | 167 | −5.794 | −30.927 | 34.740 | 1.00 | 39.67 | C |
| ATOM | 1311 | CG | PRO | H | 167 | −5.255 | −32.246 | 35.118 | 1.00 | 39.72 | C |
| ATOM | 1312 | CD | PRO | H | 167 | −6.051 | −32.648 | 36.312 | 1.00 | 40.30 | C |
| ATOM | 1313 | C | PRO | H | 167 | −7.371 | −29.377 | 35.937 | 1.00 | 39.35 | C |
| ATOM | 1314 | O | PRO | H | 167 | −8.479 | −29.901 | 36.002 | 1.00 | 39.14 | O |
| ATOM | 1315 | N | ALA | H | 168 | −7.195 | −28.069 | 35.795 | 1.00 | 38.89 | N |
| ATOM | 1316 | CA | ALA | H | 168 | −8.310 | −27.163 | 35.531 | 1.00 | 39.42 | C |
| ATOM | 1317 | CB | ALA | H | 168 | −7.858 | −25.700 | 35.602 | 1.00 | 39.19 | C |
| ATOM | 1318 | C | ALA | H | 168 | −8.901 | −27.455 | 34.165 | 1.00 | 39.58 | C |
| ATOM | 1319 | O | ALA | H | 168 | −8.168 | −27.773 | 33.226 | 1.00 | 39.22 | O |
| ATOM | 1320 | N | VAL | H | 169 | −10.231 | −27.374 | 34.083 | 1.00 | 39.88 | N |
| ATOM | 1321 | CA | VAL | H | 169 | −10.938 | −27.356 | 32.806 | 1.00 | 39.79 | C |
| ATOM | 1322 | CB | VAL | H | 169 | −12.007 | −28.483 | 32.673 | 1.00 | 39.93 | C |
| ATOM | 1323 | CG1 | VAL | H | 169 | −11.342 | −29.871 | 32.644 | 1.00 | 39.92 | C |
| ATOM | 1324 | CG2 | VAL | H | 169 | −13.065 | −28.380 | 33.779 | 1.00 | 40.37 | C |
| ATOM | 1325 | C | VAL | H | 169 | −11.600 | −26.011 | 32.585 | 1.00 | 39.53 | C |
| ATOM | 1326 | O | VAL | H | 169 | −12.221 | −25.450 | 33.496 | 1.00 | 39.34 | O |
| ATOM | 1327 | N | LEU | H | 170 | −11.461 | −25.497 | 31.369 | 1.00 | 38.97 | N |
| ATOM | 1328 | CA | LEU | H | 170 | −12.173 | −24.303 | 30.960 | 1.00 | 39.20 | C |
| ATOM | 1329 | CB | LEU | H | 170 | −11.546 | −23.757 | 29.680 | 1.00 | 39.14 | C |
| ATOM | 1330 | CG | LEU | H | 170 | −11.716 | −22.302 | 29.257 | 1.00 | 40.20 | C |
| ATOM | 1331 | CD1 | LEU | H | 170 | −11.631 | −21.281 | 30.390 | 1.00 | 40.89 | C |
| ATOM | 1332 | CD2 | LEU | H | 170 | −10.671 | −22.016 | 28.199 | 1.00 | 40.97 | C |
| ATOM | 1333 | C | LEU | H | 170 | −13.655 | −24.673 | 30.786 | 1.00 | 38.74 | C |
| ATOM | 1334 | O | LEU | H | 170 | −13.972 | −25.605 | 30.080 | 1.00 | 39.37 | O |
| ATOM | 1335 | N | GLN | H | 171 | −14.549 | −24.001 | 31.504 | 1.00 | 38.52 | N |
| ATOM | 1336 | CA | GLN | H | 171 | −15.986 | −24.295 | 31.418 | 1.00 | 38.54 | C |
| ATOM | 1337 | CB | GLN | H | 171 | −16.710 | −23.903 | 32.718 | 1.00 | 38.48 | C |
| ATOM | 1338 | CG | GLN | H | 171 | −16.230 | −24.621 | 34.019 | 1.00 | 39.29 | C |
| ATOM | 1339 | CD | GLN | H | 171 | −16.882 | −24.037 | 35.291 | 1.00 | 40.93 | C |
| ATOM | 1340 | OE1 | GLN | H | 171 | −17.812 | −24.618 | 35.839 | 1.00 | 43.16 | O |
| ATOM | 1341 | NE2 | GLN | H | 171 | −16.393 | −22.884 | 35.750 | 1.00 | 40.62 | N |
| ATOM | 1342 | C | GLN | H | 171 | −16.595 | −23.541 | 30.220 | 1.00 | 38.74 | C |
| ATOM | 1343 | O | GLN | H | 171 | −15.935 | −22.678 | 29.628 | 1.00 | 37.59 | O |
| ATOM | 1344 | N | SER | H | 172 | −17.848 | −23.867 | 29.883 | 1.00 | 38.77 | N |
| ATOM | 1345 | CA | SER | H | 172 | −18.622 | −23.152 | 28.861 | 1.00 | 39.21 | C |
| ATOM | 1346 | CB | SER | H | 172 | −20.022 | −23.738 | 28.766 | 1.00 | 39.59 | C |
| ATOM | 1347 | OG | SER | H | 172 | −20.856 | −23.221 | 29.794 | 1.00 | 40.81 | O |
| ATOM | 1348 | C | SER | H | 172 | −18.724 | −21.639 | 29.124 | 1.00 | 39.16 | C |
| ATOM | 1349 | O | SER | H | 172 | −18.750 | −20.841 | 28.184 | 1.00 | 38.72 | O |
| ATOM | 1350 | N | SER | H | 173 | −18.788 | −21.240 | 30.396 | 1.00 | 39.24 | N |
| ATOM | 1351 | CA | SER | H | 173 | −18.664 | −19.820 | 30.747 | 1.00 | 39.13 | C |
| ATOM | 1352 | CB | SER | H | 173 | −18.972 | −19.590 | 32.229 | 1.00 | 39.25 | C |
| ATOM | 1353 | OG | SER | H | 173 | −18.003 | −20.230 | 33.053 | 1.00 | 37.80 | O |
| ATOM | 1354 | C | SER | H | 173 | −17.211 | −19.558 | 30.447 | 1.00 | 39.29 | C |
| ATOM | 1355 | O | SER | H | 173 | −16.491 | −20.491 | 30.143 | 1.00 | 40.75 | O |
| ATOM | 1356 | N | GLY | H | 174 | −16.718 | −18.339 | 30.493 | 1.00 | 39.18 | N |
| ATOM | 1357 | CA | GLY | H | 174 | −15.266 | −18.241 | 30.234 | 1.00 | 39.27 | C |
| ATOM | 1358 | C | GLY | H | 174 | −14.357 | −18.693 | 31.395 | 1.00 | 38.66 | C |
| ATOM | 1359 | O | GLY | H | 174 | −13.212 | −18.264 | 31.477 | 1.00 | 39.14 | O |
| ATOM | 1360 | N | LEU | H | 175 | −14.845 | −19.573 | 32.270 | 1.00 | 37.81 | N |
| ATOM | 1361 | CA | LEU | H | 175 | −14.248 | −19.723 | 33.608 | 1.00 | 37.81 | C |
| ATOM | 1362 | CB | LEU | H | 175 | −15.272 | −19.305 | 34.672 | 1.00 | 37.37 | C |
| ATOM | 1363 | CG | LEU | H | 175 | −15.369 | −17.893 | 35.266 | 1.00 | 38.01 | C |
| ATOM | 1364 | CD1 | LEU | H | 175 | −16.824 | −17.574 | 35.599 | 1.00 | 37.08 | C |
| ATOM | 1365 | CD2 | LEU | H | 175 | −14.758 | −16.775 | 34.432 | 1.00 | 37.61 | C |
| ATOM | 1366 | C | LEU | H | 175 | −13.732 | −21.121 | 33.926 | 1.00 | 37.57 | C |
| ATOM | 1367 | O | LEU | H | 175 | −14.309 | −22.114 | 33.492 | 1.00 | 37.82 | O |
| ATOM | 1368 | N | TYR | H | 176 | −12.654 | −21.200 | 34.704 | 1.00 | 37.39 | N |
| ATOM | 1369 | CA | TYR | H | 176 | −12.089 | −22.505 | 35.069 | 1.00 | 37.10 | C |
| ATOM | 1370 | CB | TYR | H | 176 | −10.585 | −22.437 | 35.335 | 1.00 | 36.74 | C |
| ATOM | 1371 | CG | TYR | H | 176 | −9.768 | −21.908 | 34.202 | 1.00 | 33.91 | C |
| ATOM | 1372 | CD1 | TYR | H | 176 | −9.493 | −20.546 | 34.116 | 1.00 | 32.47 | C |
| ATOM | 1373 | CE1 | TYR | H | 176 | −8.740 | −20.041 | 33.108 | 1.00 | 31.95 | C |
| ATOM | 1374 | CZ | TYR | H | 176 | −8.243 | −20.894 | 32.143 | 1.00 | 32.02 | C |
| ATOM | 1375 | OH | TYR | H | 176 | −7.491 | −20.349 | 31.148 | 1.00 | 33.39 | O |
| ATOM | 1376 | CE2 | TYR | H | 176 | −8.491 | −22.246 | 32.182 | 1.00 | 30.09 | C |
| ATOM | 1377 | CD2 | TYR | H | 176 | −9.252 | −22.759 | 33.229 | 1.00 | 31.98 | C |
| ATOM | 1378 | C | TYR | H | 176 | −12.739 | −23.135 | 36.278 | 1.00 | 38.16 | C |
| ATOM | 1379 | O | TYR | H | 176 | −13.285 | −22.459 | 37.153 | 1.00 | 38.20 | O |
| ATOM | 1380 | N | SER | H | 177 | −12.641 | −24.456 | 36.311 | 1.00 | 39.46 | N |
| ATOM | 1381 | CA | SER | H | 177 | −13.087 | −25.247 | 37.427 | 1.00 | 40.52 | C |
| ATOM | 1382 | CB | SER | H | 177 | −14.456 | −25.812 | 37.112 | 1.00 | 40.09 | C |
| ATOM | 1383 | OG | SER | H | 177 | −14.973 | −26.443 | 38.254 | 1.00 | 42.82 | O |
| ATOM | 1384 | C | SER | H | 177 | −12.104 | −26.399 | 37.676 | 1.00 | 40.82 | C |
| ATOM | 1385 | O | SER | H | 177 | −11.524 | −26.936 | 36.729 | 1.00 | 40.42 | O |
| ATOM | 1386 | N | LEU | H | 178 | −11.918 | −26.763 | 38.950 | 1.00 | 41.66 | N |
| ATOM | 1387 | CA | LEU | H | 178 | −11.216 | −28.006 | 39.310 | 1.00 | 42.61 | C |
| ATOM | 1388 | CB | LEU | H | 178 | −9.688 | −27.822 | 39.308 | 1.00 | 42.89 | C |
| ATOM | 1389 | CG | LEU | H | 178 | −9.006 | −26.895 | 40.310 | 1.00 | 43.18 | C |
| ATOM | 1390 | CD1 | LEU | H | 178 | −8.885 | −27.541 | 41.690 | 1.00 | 44.49 | C |
| ATOM | 1391 | CD2 | LEU | H | 178 | −7.636 | −26.527 | 39.765 | 1.00 | 43.59 | C |
| ATOM | 1392 | C | LEU | H | 178 | −11.652 | −28.594 | 40.641 | 1.00 | 42.89 | C |
| ATOM | 1393 | O | LEU | H | 178 | −12.260 | −27.913 | 41.474 | 1.00 | 43.20 | O |
| ATOM | 1394 | N | SER | H | 179 | −11.321 | −29.866 | 40.834 | 1.00 | 43.25 | N |
| ATOM | 1395 | CA | SER | H | 179 | −11.488 | −30.522 | 42.125 | 1.00 | 43.40 | C |
| ATOM | 1396 | CB | SER | H | 179 | −12.490 | −31.661 | 42.024 | 1.00 | 43.62 | C |
| ATOM | 1397 | OG | SER | H | 179 | −13.813 | −31.153 | 41.981 | 1.00 | 43.90 | O |
| ATOM | 1398 | C | SER | H | 179 | −10.155 | −31.038 | 42.643 | 1.00 | 43.21 | C |
| ATOM | 1399 | O | SER | H | 179 | −9.256 | −31.375 | 41.870 | 1.00 | 43.21 | O |
| ATOM | 1400 | N | SER | H | 180 | −10.028 | −31.073 | 43.964 | 1.00 | 43.39 | N |
| ATOM | 1401 | CA | SER | H | 180 | −8.880 | −31.701 | 44.608 | 1.00 | 42.84 | C |
| ATOM | 1402 | CB | SER | H | 180 | −8.019 | −30.670 | 45.331 | 1.00 | 42.85 | C |
| ATOM | 1403 | OG | SER | H | 180 | −6.906 | −31.296 | 45.952 | 1.00 | 42.63 | O |
| ATOM | 1404 | C | SER | H | 180 | −9.417 | −32.718 | 45.586 | 1.00 | 42.65 | C |
| ATOM | 1405 | O | SER | H | 180 | −10.260 | −32.384 | 46.407 | 1.00 | 42.50 | O |
| ATOM | 1406 | N | VAL | H | 181 | −8.954 | −33.962 | 45.472 | 1.00 | 42.69 | N |
| ATOM | 1407 | CA | VAL | H | 181 | −9.413 | −35.039 | 46.357 | 1.00 | 42.73 | C |
| ATOM | 1408 | CB | VAL | H | 181 | −10.186 | −36.163 | 45.602 | 1.00 | 42.47 | C |
| ATOM | 1409 | CG1 | VAL | H | 181 | −11.391 | −35.599 | 44.865 | 1.00 | 41.96 | C |
| ATOM | 1410 | CG2 | VAL | H | 181 | −9.269 | −36.916 | 44.645 | 1.00 | 42.77 | C |
| ATOM | 1411 | C | VAL | H | 181 | −8.275 | −35.671 | 47.148 | 1.00 | 42.82 | C |
| ATOM | 1412 | O | VAL | H | 181 | −7.088 | −35.511 | 46.818 | 1.00 | 43.00 | O |
| ATOM | 1413 | N | VAL | H | 182 | −8.656 | −36.390 | 48.199 | 1.00 | 42.98 | N |
| ATOM | 1414 | CA | VAL | H | 182 | −7.725 | −37.175 | 48.985 | 1.00 | 42.53 | C |
| ATOM | 1415 | CB | VAL | H | 182 | −7.025 | −36.335 | 50.112 | 1.00 | 42.63 | C |
| ATOM | 1416 | CG1 | VAL | H | 182 | −8.036 | −35.636 | 51.005 | 1.00 | 42.11 | C |
| ATOM | 1417 | CG2 | VAL | H | 182 | −6.064 | −37.194 | 50.935 | 1.00 | 41.91 | C |
| ATOM | 1418 | C | VAL | H | 182 | −8.470 | −38.380 | 49.516 | 1.00 | 43.11 | C |
| ATOM | 1419 | O | VAL | H | 182 | −9.640 | −38.292 | 49.925 | 1.00 | 42.94 | O |
| ATOM | 1420 | N | THR | H | 183 | −7.802 | −39.524 | 49.444 | 1.00 | 43.25 | N |
| ATOM | 1421 | CA | THR | H | 183 | −8.308 | −40.724 | 50.071 | 1.00 | 43.37 | C |
| ATOM | 1422 | CB | THR | H | 183 | −8.054 | −41.981 | 49.226 | 1.00 | 43.52 | C |
| ATOM | 1423 | OG1 | THR | H | 183 | −6.702 | −41.958 | 48.742 | 1.00 | 45.61 | O |
| ATOM | 1424 | CG2 | THR | H | 183 | −9.020 | −42.027 | 48.056 | 1.00 | 44.05 | C |
| ATOM | 1425 | C | THR | H | 183 | −7.611 | −40.871 | 51.404 | 1.00 | 42.60 | C |
| ATOM | 1426 | O | THR | H | 183 | −6.397 | −40.639 | 51.516 | 1.00 | 42.18 | O |
| ATOM | 1427 | N | VAL | H | 184 | −8.411 | −41.229 | 52.403 | 1.00 | 41.66 | N |
| ATOM | 1428 | CA | VAL | H | 184 | −7.968 | −41.422 | 53.779 | 1.00 | 40.98 | C |
| ATOM | 1429 | CB | VAL | H | 184 | −8.293 | −40.172 | 54.659 | 1.00 | 40.91 | C |
| ATOM | 1430 | CG1 | VAL | H | 184 | −7.522 | −38.942 | 54.146 | 1.00 | 40.37 | C |
| ATOM | 1431 | CG2 | VAL | H | 184 | −9.805 | −39.890 | 54.718 | 1.00 | 39.48 | C |
| ATOM | 1432 | C | VAL | H | 184 | −8.640 | −42.686 | 54.356 | 1.00 | 41.02 | C |
| ATOM | 1433 | O | VAL | H | 184 | −9.678 | −43.128 | 53.830 | 1.00 | 40.99 | O |
| ATOM | 1434 | N | PRO | H | 185 | −8.059 | −43.282 | 55.421 | 1.00 | 40.70 | N |
| ATOM | 1435 | CA | PRO | H | 185 | −8.807 | −44.389 | 56.049 | 1.00 | 41.38 | C |
| ATOM | 1436 | CB | PRO | H | 185 | −7.887 | −44.852 | 57.192 | 1.00 | 41.38 | C |
| ATOM | 1437 | CG | PRO | H | 185 | −6.558 | −44.144 | 56.985 | 1.00 | 40.82 | C |
| ATOM | 1438 | CD | PRO | H | 185 | −6.804 | −42.956 | 56.126 | 1.00 | 40.32 | C |
| ATOM | 1439 | C | PRO | H | 185 | −10.134 | −43.870 | 56.615 | 1.00 | 41.75 | C |
| ATOM | 1440 | O | PRO | H | 185 | −10.164 | −42.742 | 57.098 | 1.00 | 41.42 | O |
| ATOM | 1441 | N | SER | H | 186 | −11.209 | −44.665 | 56.557 | 1.00 | 42.88 | N |
| ATOM | 1442 | CA | SER | H | 186 | −12.517 | −44.223 | 57.098 | 1.00 | 44.75 | C |
| ATOM | 1443 | CB | SER | H | 186 | −13.632 | −45.219 | 56.796 | 1.00 | 44.59 | C |
| ATOM | 1444 | OG | SER | H | 186 | −13.597 | −45.634 | 55.444 | 1.00 | 47.56 | O |
| ATOM | 1445 | C | SER | H | 186 | −12.459 | −43.988 | 58.603 | 1.00 | 45.21 | C |
| ATOM | 1446 | O | SER | H | 186 | −13.191 | −43.152 | 59.132 | 1.00 | 45.72 | O |
| ATOM | 1447 | N | SER | H | 187 | −11.577 | −44.727 | 59.275 | 1.00 | 46.00 | N |
| ATOM | 1448 | CA | SER | H | 187 | −11.363 | −44.615 | 60.729 | 1.00 | 46.57 | C |
| ATOM | 1449 | CB | SER | H | 187 | −10.456 | −45.750 | 61.216 | 1.00 | 46.45 | C |
| ATOM | 1450 | OG | SER | H | 187 | −9.177 | −45.677 | 60.596 | 1.00 | 46.17 | O |
| ATOM | 1451 | C | SER | H | 187 | −10.790 | −43.254 | 61.155 | 1.00 | 47.21 | C |
| ATOM | 1452 | O | SER | H | 187 | −10.888 | −42.871 | 62.329 | 1.00 | 47.16 | O |
| ATOM | 1453 | N | SER | H | 188 | −10.213 | −42.522 | 60.202 | 1.00 | 47.54 | N |
| ATOM | 1454 | CA | SER | H | 188 | −9.680 | −41.178 | 60.465 | 1.00 | 48.25 | C |
| ATOM | 1455 | CB | SER | H | 188 | −8.574 | −40.837 | 59.462 | 1.00 | 48.00 | C |
| ATOM | 1456 | OG | SER | H | 188 | −9.136 | −40.426 | 58.218 | 1.00 | 46.81 | O |
| ATOM | 1457 | C | SER | H | 188 | −10.742 | −40.064 | 60.447 | 1.00 | 49.15 | C |
| ATOM | 1458 | O | SER | H | 188 | −10.463 | −38.946 | 60.892 | 1.00 | 49.11 | O |
| ATOM | 1459 | N | LEU | H | 189 | −11.942 | −40.363 | 59.937 | 1.00 | 50.58 | N |
| ATOM | 1460 | CA | LEU | H | 189 | −12.994 | −39.337 | 59.729 | 1.00 | 52.21 | C |
| ATOM | 1461 | CB | LEU | H | 189 | −14.203 | −39.908 | 58.977 | 1.00 | 51.98 | C |
| ATOM | 1462 | CG | LEU | H | 189 | −14.059 | −40.245 | 57.485 | 1.00 | 52.48 | C |
| ATOM | 1463 | CD1 | LEU | H | 189 | −15.387 | −40.744 | 56.925 | 1.00 | 52.06 | C |
| ATOM | 1464 | CD2 | LEU | H | 189 | −13.539 | −39.055 | 56.654 | 1.00 | 51.47 | C |
| ATOM | 1465 | C | LEU | H | 189 | −13.456 | −38.631 | 61.009 | 1.00 | 53.22 | C |
| ATOM | 1466 | O | LEU | H | 189 | −13.465 | −37.398 | 61.076 | 1.00 | 53.57 | O |
| ATOM | 1467 | N | GLY | H | 190 | −13.834 | −39.410 | 62.020 | 1.00 | 54.35 | N |
| ATOM | 1468 | CA | GLY | H | 190 | −14.202 | −38.848 | 63.332 | 1.00 | 55.51 | C |
| ATOM | 1469 | C | GLY | H | 190 | −13.063 | −38.181 | 64.102 | 1.00 | 56.10 | C |
| ATOM | 1470 | O | GLY | H | 190 | −13.308 | −37.385 | 65.017 | 1.00 | 56.51 | O |
| ATOM | 1471 | N | THR | H | 191 | −11.829 | −38.475 | 63.687 | 1.00 | 56.21 | N |
| ATOM | 1472 | CA | THR | H | 191 | −10.601 | −38.207 | 64.442 | 1.00 | 56.54 | C |
| ATOM | 1473 | CB | THR | H | 191 | −9.695 | −39.475 | 64.427 | 1.00 | 56.56 | C |
| ATOM | 1474 | OG1 | THR | H | 191 | −10.349 | −40.541 | 65.124 | 1.00 | 57.85 | O |
| ATOM | 1475 | CG2 | THR | H | 191 | −8.338 | −39.217 | 65.057 | 1.00 | 56.63 | C |
| ATOM | 1476 | C | THR | H | 191 | −9.764 | −37.044 | 63.894 | 1.00 | 56.44 | C |
| ATOM | 1477 | O | THR | H | 191 | −9.145 | −36.293 | 64.655 | 1.00 | 56.51 | O |
| ATOM | 1478 | N | GLN | H | 192 | −9.704 | −36.921 | 62.577 | 1.00 | 55.83 | N |
| ATOM | 1479 | CA | GLN | H | 192 | −8.849 | −35.920 | 61.965 | 1.00 | 55.53 | C |
| ATOM | 1480 | CB | GLN | H | 192 | −7.863 | −36.602 | 61.023 | 1.00 | 55.67 | C |
| ATOM | 1481 | CG | GLN | H | 192 | −6.466 | −36.784 | 61.618 | 1.00 | 56.96 | C |
| ATOM | 1482 | CD | GLN | H | 192 | −5.545 | −35.626 | 61.281 | 1.00 | 57.29 | C |
| ATOM | 1483 | OE1 | GLN | H | 192 | −5.819 | −34.849 | 60.354 | 1.00 | 56.87 | O |
| ATOM | 1484 | NE2 | GLN | H | 192 | −4.446 | −35.503 | 62.026 | 1.00 | 57.41 | N |
| ATOM | 1485 | C | GLN | H | 192 | −9.694 | −34.911 | 61.213 | 1.00 | 54.98 | C |
| ATOM | 1486 | O | GLN | H | 192 | −10.736 | −35.270 | 60.668 | 1.00 | 55.13 | O |
| ATOM | 1487 | N | THR | H | 193 | −9.257 | −33.653 | 61.199 | 1.00 | 54.32 | N |
| ATOM | 1488 | CA | THR | H | 193 | −9.921 | −32.623 | 60.392 | 1.00 | 53.84 | C |
| ATOM | 1489 | CB | THR | H | 193 | −9.943 | −31.210 | 61.064 | 1.00 | 53.98 | C |
| ATOM | 1490 | OG1 | THR | H | 193 | −8.605 | −30.717 | 61.217 | 1.00 | 54.11 | O |
| ATOM | 1491 | CG2 | THR | H | 193 | −10.648 | −31.242 | 62.419 | 1.00 | 54.01 | C |
| ATOM | 1492 | C | THR | H | 193 | −9.256 | −32.505 | 59.018 | 1.00 | 53.18 | C |
| ATOM | 1493 | O | THR | H | 193 | −8.058 | −32.750 | 58.866 | 1.00 | 53.04 | O |
| ATOM | 1494 | N | TYR | H | 194 | −10.047 | −32.121 | 58.024 | 1.00 | 52.36 | N |
| ATOM | 1495 | CA | TYR | H | 194 | −9.544 | −31.964 | 56.667 | 1.00 | 51.25 | C |
| ATOM | 1496 | CB | TYR | H | 194 | −10.013 | −33.126 | 55.779 | 1.00 | 50.87 | C |
| ATOM | 1497 | CG | TYR | H | 194 | −9.497 | −34.467 | 56.256 | 1.00 | 50.01 | C |
| ATOM | 1498 | CD1 | TYR | H | 194 | −10.332 | −35.355 | 56.925 | 1.00 | 48.45 | C |
| ATOM | 1499 | CE1 | TYR | H | 194 | −9.859 | −36.576 | 57.382 | 1.00 | 48.48 | C |
| ATOM | 1500 | CZ | TYR | H | 194 | −8.526 | −36.923 | 57.182 | 1.00 | 48.38 | C |
| ATOM | 1501 | OH | TYR | H | 194 | −8.052 | −38.141 | 57.644 | 1.00 | 46.61 | O |
| ATOM | 1502 | CE2 | TYR | H | 194 | −7.671 | −36.051 | 56.525 | 1.00 | 48.42 | C |
| ATOM | 1503 | CD2 | TYR | H | 194 | −8.159 | −34.830 | 56.067 | 1.00 | 48.95 | C |
| ATOM | 1504 | C | TYR | H | 194 | −9.925 | −30.595 | 56.101 | 1.00 | 50.85 | C |
| ATOM | 1505 | O | TYR | H | 194 | −11.105 | −30.273 | 55.941 | 1.00 | 50.86 | O |
| ATOM | 1506 | N | ILE | H | 195 | −8.906 | −29.785 | 55.843 | 1.00 | 50.57 | N |
| ATOM | 1507 | CA | ILE | H | 195 | −9.088 | −28.452 | 55.280 | 1.00 | 50.47 | C |
| ATOM | 1508 | CB | ILE | H | 195 | −8.619 | −27.338 | 56.255 | 1.00 | 50.57 | C |
| ATOM | 1509 | CG1 | ILE | H | 195 | −9.358 | −27.439 | 57.593 | 1.00 | 50.37 | C |
| ATOM | 1510 | CD1 | ILE | H | 195 | −8.835 | −26.515 | 58.670 | 1.00 | 51.29 | C |
| ATOM | 1511 | CG2 | ILE | H | 195 | −8.810 | −25.951 | 55.614 | 1.00 | 50.91 | C |
| ATOM | 1512 | C | ILE | H | 195 | −8.306 | −28.336 | 53.979 | 1.00 | 50.15 | C |
| ATOM | 1513 | O | ILE | H | 195 | −7.117 | −28.644 | 53.932 | 1.00 | 49.59 | O |
| ATOM | 1514 | N | CYS | H | 196 | −8.988 | −27.912 | 52.917 | 1.00 | 50.27 | N |
| ATOM | 1515 | CA | CYS | H | 196 | −8.303 | −27.569 | 51.681 | 1.00 | 49.90 | C |
| ATOM | 1516 | CB | CYS | H | 196 | −9.165 | −27.909 | 50.462 | 1.00 | 49.88 | C |
| ATOM | 1517 | SG | CYS | H | 196 | −10.739 | −27.043 | 50.438 | 1.00 | 50.40 | S |
| ATOM | 1518 | C | CYS | H | 196 | −7.944 | −26.083 | 51.724 | 1.00 | 49.69 | C |
| ATOM | 1519 | O | CYS | H | 196 | −8.794 | −25.238 | 51.988 | 1.00 | 49.78 | O |
| ATOM | 1520 | N | ASN | H | 197 | −6.671 | −25.781 | 51.496 | 1.00 | 49.74 | N |
| ATOM | 1521 | CA | ASN | H | 197 | −6.185 | −24.400 | 51.445 | 1.00 | 49.77 | C |
| ATOM | 1522 | CB | ASN | H | 197 | −4.825 | −24.273 | 52.146 | 1.00 | 49.48 | C |
| ATOM | 1523 | CG | ASN | H | 197 | −4.752 | −25.083 | 53.432 | 1.00 | 48.39 | C |
| ATOM | 1524 | OD1 | ASN | H | 197 | −4.302 | −26.224 | 53.422 | 1.00 | 46.98 | O |
| ATOM | 1525 | ND2 | ASN | H | 197 | −5.227 | −24.506 | 54.536 | 1.00 | 46.67 | N |
| ATOM | 1526 | C | ASN | H | 197 | −6.081 | −23.962 | 49.984 | 1.00 | 50.37 | C |
| ATOM | 1527 | O | ASN | H | 197 | −5.137 | −24.343 | 49.265 | 1.00 | 50.28 | O |
| ATOM | 1528 | N | VAL | H | 198 | −7.071 | −23.186 | 49.547 | 1.00 | 50.92 | N |
| ATOM | 1529 | CA | VAL | H | 198 | −7.156 | −22.745 | 48.158 | 1.00 | 51.10 | C |
| ATOM | 1530 | CB | VAL | H | 198 | −8.590 | −22.833 | 47.600 | 1.00 | 51.11 | C |
| ATOM | 1531 | CG1 | VAL | H | 198 | −8.612 | −22.410 | 46.120 | 1.00 | 50.58 | C |
| ATOM | 1532 | CG2 | VAL | H | 198 | −9.144 | −24.232 | 47.762 | 1.00 | 50.57 | C |
| ATOM | 1533 | C | VAL | H | 198 | −6.678 | −21.314 | 48.049 | 1.00 | 51.62 | C |
| ATOM | 1534 | O | VAL | H | 198 | −7.269 | −20.404 | 48.640 | 1.00 | 51.34 | O |
| ATOM | 1535 | N | ASN | H | 199 | −5.603 | −21.119 | 47.296 | 1.00 | 52.33 | N |
| ATOM | 1536 | CA | ASN | H | 199 | −5.128 | −19.777 | 47.050 | 1.00 | 53.66 | C |
| ATOM | 1537 | CB | ASN | H | 199 | −3.764 | −19.527 | 47.701 | 1.00 | 54.14 | C |
| ATOM | 1538 | CG | ASN | H | 199 | −3.405 | −18.042 | 47.769 | 1.00 | 55.98 | C |
| ATOM | 1539 | OD1 | ASN | H | 199 | −4.245 | −17.162 | 47.535 | 1.00 | 58.86 | O |
| ATOM | 1540 | ND2 | ASN | H | 199 | −2.149 | −17.759 | 48.084 | 1.00 | 57.20 | N |
| ATOM | 1541 | C | ASN | H | 199 | −5.144 | −19.396 | 45.565 | 1.00 | 53.89 | C |
| ATOM | 1542 | O | ASN | H | 199 | −4.668 | −20.143 | 44.694 | 1.00 | 53.54 | O |
| ATOM | 1543 | N | HIS | H | 200 | −5.721 | −18.223 | 45.314 | 1.00 | 54.27 | N |
| ATOM | 1544 | CA | HIS | H | 200 | −5.816 | −17.643 | 43.994 | 1.00 | 54.98 | C |
| ATOM | 1545 | CB | HIS | H | 200 | −7.288 | −17.577 | 43.559 | 1.00 | 54.95 | C |
| ATOM | 1546 | CG | HIS | H | 200 | −7.479 | −17.109 | 42.152 | 1.00 | 54.88 | C |
| ATOM | 1547 | ND1 | HIS | H | 200 | −8.301 | −16.052 | 41.827 | 1.00 | 54.61 | N |
| ATOM | 1548 | CE1 | HIS | H | 200 | −8.255 | −15.847 | 40.523 | 1.00 | 53.36 | C |
| ATOM | 1549 | NE2 | HIS | H | 200 | −7.430 | −16.730 | 39.991 | 1.00 | 53.33 | N |
| ATOM | 1550 | CD2 | HIS | H | 200 | −6.927 | −17.528 | 40.989 | 1.00 | 53.88 | C |
| ATOM | 1551 | C | HIS | H | 200 | −5.171 | −16.256 | 44.022 | 1.00 | 55.54 | C |
| ATOM | 1552 | O | HIS | H | 200 | −5.826 | −15.260 | 44.338 | 1.00 | 55.60 | O |
| ATOM | 1553 | N | LYS | H | 201 | −3.875 | −16.206 | 43.709 | 1.00 | 56.34 | N |
| ATOM | 1554 | CA | LYS | H | 201 | −3.113 | −14.944 | 43.719 | 1.00 | 57.21 | C |
| ATOM | 1555 | CB | LYS | H | 201 | −1.624 | −15.173 | 43.405 | 1.00 | 57.41 | C |
| ATOM | 1556 | CG | LYS | H | 201 | −0.685 | −14.911 | 44.583 | 1.00 | 59.04 | C |
| ATOM | 1557 | CD | LYS | H | 201 | 0.736 | −14.619 | 44.094 | 1.00 | 61.37 | C |
| ATOM | 1558 | CE | LYS | H | 201 | 1.652 | −14.108 | 45.216 | 1.00 | 62.67 | C |
| ATOM | 1559 | NZ | LYS | H | 201 | 2.262 | −15.195 | 46.051 | 1.00 | 62.52 | N |
| ATOM | 1560 | C | LYS | H | 201 | −3.686 | −13.824 | 42.823 | 1.00 | 57.36 | C |
| ATOM | 1561 | O | LYS | H | 201 | −3.783 | −12.671 | 43.275 | 1.00 | 57.33 | O |
| ATOM | 1562 | N | PRO | H | 202 | −4.082 | −14.151 | 41.567 | 1.00 | 57.44 | N |
| ATOM | 1563 | CA | PRO | H | 202 | −4.564 | −13.084 | 40.673 | 1.00 | 57.22 | C |
| ATOM | 1564 | CB | PRO | H | 202 | −5.092 | −13.856 | 39.466 | 1.00 | 57.20 | C |
| ATOM | 1565 | CG | PRO | H | 202 | −4.257 | −15.086 | 39.427 | 1.00 | 57.15 | C |
| ATOM | 1566 | CD | PRO | H | 202 | −4.064 | −15.459 | 40.875 | 1.00 | 57.34 | C |
| ATOM | 1567 | C | PRO | H | 202 | −5.661 | −12.213 | 41.272 | 1.00 | 57.23 | C |
| ATOM | 1568 | O | PRO | H | 202 | −5.699 | −11.021 | 40.993 | 1.00 | 57.47 | O |
| ATOM | 1569 | N | SER | H | 203 | −6.534 | −12.793 | 42.090 | 1.00 | 57.46 | N |
| ATOM | 1570 | CA | SER | H | 203 | −7.595 | −12.028 | 42.743 | 1.00 | 57.96 | C |
| ATOM | 1571 | CB | SER | H | 203 | −8.940 | −12.736 | 42.576 | 1.00 | 57.72 | C |
| ATOM | 1572 | OG | SER | H | 203 | −9.012 | −13.883 | 43.404 | 1.00 | 56.81 | O |
| ATOM | 1573 | C | SER | H | 203 | −7.343 | −11.789 | 44.233 | 1.00 | 58.76 | C |
| ATOM | 1574 | O | SER | H | 203 | −8.209 | −11.247 | 44.921 | 1.00 | 58.84 | O |
| ATOM | 1575 | N | ASN | H | 204 | −6.169 | −12.191 | 44.724 | 1.00 | 59.84 | N |
| ATOM | 1576 | CA | ASN | H | 204 | −5.881 | −12.229 | 46.176 | 1.00 | 61.06 | C |
| ATOM | 1577 | CB | ASN | H | 204 | −5.489 | −10.849 | 46.717 | 1.00 | 60.99 | C |
| ATOM | 1578 | CG | ASN | H | 204 | −4.176 | −10.356 | 46.152 | 1.00 | 62.15 | C |
| ATOM | 1579 | OD1 | ASN | H | 204 | −3.129 | −10.992 | 46.319 | 1.00 | 62.44 | O |
| ATOM | 1580 | ND2 | ASN | H | 204 | −4.221 | −9.208 | 45.476 | 1.00 | 63.26 | N |
| ATOM | 1581 | C | ASN | H | 204 | −7.029 | −12.816 | 47.003 | 1.00 | 61.44 | C |
| ATOM | 1582 | O | ASN | H | 204 | −7.550 | −12.168 | 47.909 | 1.00 | 61.66 | O |
| ATOM | 1583 | N | THR | H | 205 | −7.434 | −14.034 | 46.661 | 1.00 | 62.05 | N |
| ATOM | 1584 | CA | THR | H | 205 | −8.430 | −14.767 | 47.441 | 1.00 | 62.55 | C |
| ATOM | 1585 | CB | THR | H | 205 | −9.638 | −15.186 | 46.571 | 1.00 | 62.46 | C |
| ATOM | 1586 | OG1 | THR | H | 205 | −10.310 | −14.013 | 46.105 | 1.00 | 63.24 | O |
| ATOM | 1587 | CG2 | THR | H | 205 | −10.636 | −16.028 | 47.362 | 1.00 | 63.09 | C |
| ATOM | 1588 | C | THR | H | 205 | −7.751 | −15.981 | 48.061 | 1.00 | 62.69 | C |
| ATOM | 1589 | O | THR | H | 205 | −7.118 | −16.773 | 47.365 | 1.00 | 62.58 | O |
| ATOM | 1590 | N | LYS | H | 206 | −7.851 | −16.087 | 49.380 | 1.00 | 63.17 | N |
| ATOM | 1591 | CA | LYS | H | 206 | −7.391 | −17.266 | 50.105 | 1.00 | 63.49 | C |
| ATOM | 1592 | CB | LYS | H | 206 | −6.287 | −16.926 | 51.108 | 1.00 | 63.42 | C |
| ATOM | 1593 | CG | LYS | H | 206 | −5.129 | −16.154 | 50.502 | 1.00 | 64.56 | C |
| ATOM | 1594 | CD | LYS | H | 206 | −3.986 | −15.978 | 51.478 | 1.00 | 65.66 | C |
| ATOM | 1595 | CE | LYS | H | 206 | −3.130 | −14.786 | 51.077 | 1.00 | 66.75 | C |
| ATOM | 1596 | NZ | LYS | H | 206 | −1.751 | −14.907 | 51.629 | 1.00 | 67.34 | N |
| ATOM | 1597 | C | LYS | H | 206 | −8.590 | −17.830 | 50.819 | 1.00 | 63.45 | C |
| ATOM | 1598 | O | LYS | H | 206 | −9.285 | −17.116 | 51.538 | 1.00 | 63.49 | O |
| ATOM | 1599 | N | VAL | H | 207 | −8.849 | −19.108 | 50.583 | 1.00 | 63.61 | N |
| ATOM | 1600 | CA | VAL | H | 207 | −9.956 | −19.800 | 51.219 | 1.00 | 63.65 | C |
| ATOM | 1601 | CB | VAL | H | 207 | −11.110 | −20.072 | 50.229 | 1.00 | 63.67 | C |
| ATOM | 1602 | CG1 | VAL | H | 207 | −12.104 | −21.061 | 50.814 | 1.00 | 63.47 | C |
| ATOM | 1603 | CG2 | VAL | H | 207 | −11.812 | −18.760 | 49.848 | 1.00 | 63.61 | C |
| ATOM | 1604 | C | VAL | H | 207 | −9.456 | −21.104 | 51.830 | 1.00 | 63.89 | C |
| ATOM | 1605 | O | VAL | H | 207 | −8.805 | −21.913 | 51.158 | 1.00 | 63.76 | O |
| ATOM | 1606 | N | ASP | H | 208 | −9.744 | −21.275 | 53.118 | 1.00 | 63.93 | N |
| ATOM | 1607 | CA | ASP | H | 208 | −9.480 | −22.518 | 53.820 | 1.00 | 64.05 | C |
| ATOM | 1608 | CB | ASP | H | 208 | −8.670 | −22.255 | 55.090 | 1.00 | 64.08 | C |
| ATOM | 1609 | CG | ASP | H | 208 | −7.386 | −21.498 | 54.813 | 1.00 | 64.63 | C |
| ATOM | 1610 | OD1 | ASP | H | 208 | −6.438 | −22.095 | 54.257 | 1.00 | 64.89 | O |
| ATOM | 1611 | OD2 | ASP | H | 208 | −7.325 | −20.295 | 55.150 | 1.00 | 66.29 | O |
| ATOM | 1612 | C | ASP | H | 208 | −10.826 | −23.128 | 54.150 | 1.00 | 63.88 | C |
| ATOM | 1613 | O | ASP | H | 208 | −11.508 | −22.662 | 55.054 | 1.00 | 64.01 | O |
| ATOM | 1614 | N | LYS | H | 209 | −11.219 | −24.149 | 53.396 | 1.00 | 63.88 | N |
| ATOM | 1615 | CA | LYS | H | 209 | −12.529 | −24.770 | 53.573 | 1.00 | 63.88 | C |
| ATOM | 1616 | CB | LYS | H | 209 | −13.248 | −24.926 | 52.222 | 1.00 | 63.76 | C |
| ATOM | 1617 | CG | LYS | H | 209 | −14.614 | −25.608 | 52.275 | 1.00 | 63.34 | C |
| ATOM | 1618 | CD | LYS | H | 209 | −15.670 | −24.779 | 52.996 | 1.00 | 63.59 | C |
| ATOM | 1619 | CE | LYS | H | 209 | −16.852 | −25.664 | 53.379 | 1.00 | 64.08 | C |
| ATOM | 1620 | NZ | LYS | H | 209 | −17.841 | −24.986 | 54.268 | 1.00 | 63.81 | N |
| ATOM | 1621 | C | LYS | H | 209 | −12.438 | −26.102 | 54.321 | 1.00 | 64.20 | C |
| ATOM | 1622 | O | LYS | H | 209 | −11.812 | −27.064 | 53.856 | 1.00 | 64.14 | O |
| ATOM | 1623 | N | ARG | H | 210 | −13.073 | −26.124 | 55.488 | 1.00 | 64.58 | N |
| ATOM | 1624 | CA | ARG | H | 210 | −13.191 | −27.306 | 56.320 | 1.00 | 65.07 | C |
| ATOM | 1625 | CB | ARG | H | 210 | −13.678 | −26.900 | 57.716 | 1.00 | 65.40 | C |
| ATOM | 1626 | CG | ARG | H | 210 | −13.376 | −27.903 | 58.824 | 1.00 | 67.52 | C |
| ATOM | 1627 | CD | ARG | H | 210 | −13.379 | −27.222 | 60.196 | 1.00 | 70.89 | C |
| ATOM | 1628 | NE | ARG | H | 210 | −13.218 | −28.184 | 61.295 | 1.00 | 73.79 | N |
| ATOM | 1629 | CZ | ARG | H | 210 | −13.055 | −27.860 | 62.581 | 1.00 | 74.93 | C |
| ATOM | 1630 | NH1 | ARG | H | 210 | −13.022 | −26.585 | 62.963 | 1.00 | 75.43 | N |
| ATOM | 1631 | NH2 | ARG | H | 210 | −12.916 | −28.818 | 63.494 | 1.00 | 75.33 | N |
| ATOM | 1632 | C | ARG | H | 210 | −14.167 | −28.281 | 55.671 | 1.00 | 64.82 | C |
| ATOM | 1633 | O | ARG | H | 210 | −15.312 | −27.928 | 55.387 | 1.00 | 65.03 | O |
| ATOM | 1634 | N | VAL | H | 211 | −13.702 | −29.500 | 55.425 | 1.00 | 64.62 | N |
| ATOM | 1635 | CA | VAL | H | 211 | −14.533 | −30.532 | 54.807 | 1.00 | 64.68 | C |
| ATOM | 1636 | CB | VAL | H | 211 | −13.797 | −31.233 | 53.630 | 1.00 | 64.61 | C |
| ATOM | 1637 | CG1 | VAL | H | 211 | −14.757 | −32.106 | 52.820 | 1.00 | 63.84 | C |
| ATOM | 1638 | CG2 | VAL | H | 211 | −13.121 | −30.211 | 52.739 | 1.00 | 63.54 | C |
| ATOM | 1639 | C | VAL | H | 211 | −14.929 | −31.559 | 55.866 | 1.00 | 65.00 | C |
| ATOM | 1640 | O | VAL | H | 211 | −14.063 | −32.177 | 56.485 | 1.00 | 65.00 | O |
| ATOM | 1641 | N | GLU | H | 212 | −16.233 | −31.728 | 56.072 | 1.00 | 65.57 | N |
| ATOM | 1642 | CA | GLU | H | 212 | −16.750 | −32.624 | 57.110 | 1.00 | 66.35 | C |
| ATOM | 1643 | CB | GLU | H | 212 | −17.423 | −31.813 | 58.222 | 1.00 | 66.61 | C |
| ATOM | 1644 | CG | GLU | H | 212 | −16.461 | −31.054 | 59.133 | 1.00 | 67.32 | C |
| ATOM | 1645 | CD | GLU | H | 212 | −17.186 | −30.183 | 60.149 | 1.00 | 68.22 | C |
| ATOM | 1646 | OE1 | GLU | H | 212 | −17.907 | −29.246 | 59.736 | 1.00 | 67.95 | O |
| ATOM | 1647 | OE2 | GLU | H | 212 | −17.032 | −30.440 | 61.362 | 1.00 | 68.23 | O |
| ATOM | 1648 | C | GLU | H | 212 | −17.737 | −33.662 | 56.563 | 1.00 | 66.87 | C |
| ATOM | 1649 | O | GLU | H | 212 | −18.414 | −33.404 | 55.561 | 1.00 | 66.63 | O |
| ATOM | 1650 | N | PRO | H | 213 | −17.831 | −34.836 | 57.228 | 1.00 | 67.39 | N |
| ATOM | 1651 | CA | PRO | H | 213 | −18.802 | −35.860 | 56.841 | 1.00 | 67.83 | C |
| ATOM | 1652 | CB | PRO | H | 213 | −18.674 | −36.904 | 57.955 | 1.00 | 67.89 | C |
| ATOM | 1653 | CG | PRO | H | 213 | −17.290 | −36.737 | 58.464 | 1.00 | 67.33 | C |
| ATOM | 1654 | CD | PRO | H | 213 | −17.021 | −35.267 | 58.385 | 1.00 | 67.51 | C |
| ATOM | 1655 | C | PRO | H | 213 | −20.211 | −35.292 | 56.828 | 1.00 | 68.24 | C |
| ATOM | 1656 | O | PRO | H | 213 | −20.525 | −34.419 | 57.636 | 1.00 | 68.16 | O |
| ATOM | 1657 | N | LYS | H | 214 | −21.031 | −35.785 | 55.902 | 1.00 | 68.87 | N |
| ATOM | 1658 | CA | LYS | H | 214 | −22.398 | −35.292 | 55.680 | 1.00 | 69.56 | C |
| ATOM | 1659 | CB | LYS | H | 214 | −23.411 | −36.083 | 56.523 | 1.00 | 69.48 | C |
| ATOM | 1660 | CG | LYS | H | 214 | −24.845 | −35.961 | 56.026 | 1.00 | 70.32 | C |
| ATOM | 1661 | CD | LYS | H | 214 | −25.813 | −36.752 | 56.888 | 1.00 | 70.52 | C |
| ATOM | 1662 | CE | LYS | H | 214 | −27.215 | −36.190 | 56.773 | 1.00 | 70.25 | C |
| ATOM | 1663 | NZ | LYS | H | 214 | −27.301 | −34.832 | 57.369 | 1.00 | 70.03 | N |
| ATOM | 1664 | C | LYS | H | 214 | −22.543 | −33.779 | 55.918 | 1.00 | 69.64 | C |
| ATOM | 1665 | O | LYS | H | 214 | −23.368 | −33.116 | 55.289 | 1.00 | 69.80 | O |
| TER | 1666 | | LYS | H | 214 | | | | | | |
| ATOM | 1666 | N | ASP | L | 1 | 5.398 | −25.805 | 4.404 | 1.00 | 42.36 | N |
| ATOM | 1667 | CA | ASP | L | 1 | 5.835 | −26.333 | 5.731 | 1.00 | 42.48 | C |
| ATOM | 1668 | CB | ASP | L | 1 | 4.812 | −25.980 | 6.826 | 1.00 | 42.63 | C |
| ATOM | 1669 | CG | ASP | L | 1 | 4.576 | −24.467 | 6.958 | 1.00 | 44.20 | C |
| ATOM | 1670 | OD1 | ASP | L | 1 | 4.884 | −23.696 | 6.017 | 1.00 | 45.92 | O |
| ATOM | 1671 | OD2 | ASP | L | 1 | 4.058 | −24.039 | 8.008 | 1.00 | 46.35 | O |
| ATOM | 1672 | C | ASP | L | 1 | 6.031 | −27.845 | 5.626 | 1.00 | 41.68 | C |
| ATOM | 1673 | O | ASP | L | 1 | 5.303 | −28.517 | 4.888 | 1.00 | 42.53 | O |
| ATOM | 1674 | N | VAL | L | 2 | 7.037 | −28.370 | 6.314 | 1.00 | 39.97 | N |
| ATOM | 1675 | CA | VAL | L | 2 | 7.290 | −29.795 | 6.287 | 1.00 | 38.49 | C |
| ATOM | 1676 | CB | VAL | L | 2 | 8.800 | −30.116 | 6.375 | 1.00 | 38.91 | C |
| ATOM | 1677 | CG1 | VAL | L | 2 | 9.035 | −31.594 | 6.166 | 1.00 | 39.13 | C |
| ATOM | 1678 | CG2 | VAL | L | 2 | 9.580 | −29.339 | 5.326 | 1.00 | 39.28 | C |
| ATOM | 1679 | C | VAL | L | 2 | 6.500 | −30.418 | 7.445 | 1.00 | 37.51 | C |
| ATOM | 1680 | O | VAL | L | 2 | 6.714 | −30.057 | 8.610 | 1.00 | 36.89 | O |
| ATOM | 1681 | N | VAL | L | 3 | 5.561 | −31.310 | 7.113 | 1.00 | 35.77 | N |
| ATOM | 1682 | CA | VAL | L | 3 | 4.686 | −31.955 | 8.096 | 1.00 | 34.28 | C |
| ATOM | 1683 | CB | VAL | L | 3 | 3.358 | −32.514 | 7.447 | 1.00 | 34.64 | C |
| ATOM | 1684 | CG1 | VAL | L | 3 | 2.550 | −33.344 | 8.456 | 1.00 | 34.86 | C |
| ATOM | 1685 | CG2 | VAL | L | 3 | 2.471 | −31.374 | 6.880 | 1.00 | 35.41 | C |
| ATOM | 1686 | C | VAL | L | 3 | 5.454 | −33.103 | 8.718 | 1.00 | 33.14 | C |
| ATOM | 1687 | O | VAL | L | 3 | 6.019 | −33.919 | 8.011 | 1.00 | 32.88 | O |
| ATOM | 1688 | N | MET | L | 4 | 5.461 | −33.172 | 10.042 | 1.00 | 32.23 | N |
| ATOM | 1689 | CA | MET | L | 4 | 6.135 | −34.249 | 10.753 | 1.00 | 31.31 | C |
| ATOM | 1690 | CB | MET | L | 4 | 7.039 | −33.647 | 11.846 | 1.00 | 31.66 | C |
| ATOM | 1691 | CG | MET | L | 4 | 8.071 | −32.664 | 11.357 | 1.00 | 32.39 | C |
| ATOM | 1692 | SD | MET | L | 4 | 9.310 | −33.474 | 10.358 | 1.00 | 33.13 | S |
| ATOM | 1693 | CE | MET | L | 4 | 8.766 | −32.863 | 8.799 | 1.00 | 40.68 | C |
| ATOM | 1694 | C | MET | L | 4 | 5.084 | −35.122 | 11.411 | 1.00 | 30.85 | C |
| ATOM | 1695 | O | MET | L | 4 | 4.254 | −34.618 | 12.157 | 1.00 | 31.11 | O |
| ATOM | 1696 | N | THR | L | 5 | 5.131 | −36.428 | 11.178 | 1.00 | 30.72 | N |
| ATOM | 1697 | CA | THR | L | 5 | 4.117 | −37.337 | 11.727 | 1.00 | 30.75 | C |
| ATOM | 1698 | CB | THR | L | 5 | 3.250 | −37.966 | 10.618 | 1.00 | 31.25 | C |
| ATOM | 1699 | OG1 | THR | L | 5 | 2.625 | −36.911 | 9.875 | 1.00 | 32.90 | O |
| ATOM | 1700 | CG2 | THR | L | 5 | 2.145 | −38.871 | 11.226 | 1.00 | 32.35 | C |
| ATOM | 1701 | C | THR | L | 5 | 4.749 | −38.444 | 12.563 | 1.00 | 29.90 | C |
| ATOM | 1702 | O | THR | L | 5 | 5.617 | −39.175 | 12.097 | 1.00 | 29.45 | O |
| ATOM | 1703 | N | GLN | L | 6 | 4.305 | −38.553 | 13.807 | 1.00 | 29.24 | N |
| ATOM | 1704 | CA | GLN | L | 6 | 4.840 | −39.562 | 14.710 | 1.00 | 29.69 | C |
| ATOM | 1705 | CB | GLN | L | 6 | 5.152 | −38.921 | 16.067 | 1.00 | 29.49 | C |
| ATOM | 1706 | CG | GLN | L | 6 | 6.236 | −37.853 | 16.010 | 1.00 | 29.90 | C |
| ATOM | 1707 | CD | GLN | L | 6 | 6.600 | −37.303 | 17.377 | 1.00 | 30.60 | C |
| ATOM | 1708 | OE1 | GLN | L | 6 | 6.691 | −36.085 | 17.557 | 1.00 | 28.11 | O |
| ATOM | 1709 | NE2 | GLN | L | 6 | 6.803 | −38.197 | 18.355 | 1.00 | 27.49 | N |
| ATOM | 1710 | C | GLN | L | 6 | 3.844 | −40.699 | 14.900 | 1.00 | 29.88 | C |
| ATOM | 1711 | O | GLN | L | 6 | 2.650 | −40.460 | 14.985 | 1.00 | 29.61 | O |
| ATOM | 1712 | N | THR | L | 7 | 4.336 | −41.933 | 14.975 | 1.00 | 30.36 | N |
| ATOM | 1713 | CA | THR | L | 7 | 3.498 | −43.051 | 15.389 | 1.00 | 30.89 | C |
| ATOM | 1714 | CB | THR | L | 7 | 3.086 | −43.972 | 14.191 | 1.00 | 30.61 | C |
| ATOM | 1715 | OG1 | THR | L | 7 | 4.262 | −44.514 | 13.606 | 1.00 | 33.32 | O |
| ATOM | 1716 | CG2 | THR | L | 7 | 2.317 | −43.189 | 13.071 | 1.00 | 31.11 | C |
| ATOM | 1717 | C | THR | L | 7 | 4.287 | −43.884 | 16.414 | 1.00 | 31.13 | C |
| ATOM | 1718 | O | THR | L | 7 | 5.502 | −44.064 | 16.268 | 1.00 | 30.62 | O |
| ATOM | 1719 | N | PRO | L | 8 | 3.601 | −44.399 | 17.450 | 1.00 | 31.31 | N |
| ATOM | 1720 | CA | PRO | L | 8 | 2.193 | −44.163 | 17.775 | 1.00 | 31.12 | C |
| ATOM | 1721 | CB | PRO | L | 8 | 1.864 | −45.319 | 18.716 | 1.00 | 31.35 | C |
| ATOM | 1722 | CG | PRO | L | 8 | 3.156 | −45.530 | 19.463 | 1.00 | 31.78 | C |
| ATOM | 1723 | CD | PRO | L | 8 | 4.218 | −45.379 | 18.366 | 1.00 | 31.17 | C |
| ATOM | 1724 | C | PRO | L | 8 | 2.100 | −42.837 | 18.515 | 1.00 | 31.66 | C |
| ATOM | 1725 | O | PRO | L | 8 | 3.131 | −42.159 | 18.656 | 1.00 | 31.54 | O |
| ATOM | 1726 | N | LEU | L | 9 | 0.893 | −42.458 | 18.950 | 1.00 | 31.55 | N |
| ATOM | 1727 | CA | LEU | L | 9 | 0.688 | −41.256 | 19.748 | 1.00 | 32.30 | C |
| ATOM | 1728 | CB | LEU | L | 9 | −0.654 | −40.576 | 19.422 | 1.00 | 32.61 | C |
| ATOM | 1729 | CG | LEU | L | 9 | −0.914 | −40.048 | 17.995 | 1.00 | 35.66 | C |
| ATOM | 1730 | CD1 | LEU | L | 9 | −2.201 | −39.195 | 17.984 | 1.00 | 37.91 | C |
| ATOM | 1731 | CD2 | LEU | L | 9 | 0.272 | −39.257 | 17.405 | 1.00 | 36.54 | C |
| ATOM | 1732 | C | LEU | L | 9 | 0.749 | −41.554 | 21.244 | 1.00 | 32.52 | C |
| ATOM | 1733 | O | LEU | L | 9 | 1.059 | −40.668 | 22.046 | 1.00 | 32.13 | O |
| ATOM | 1734 | N | SER | L | 10 | 0.409 | −42.787 | 21.609 | 1.00 | 32.57 | N |
| ATOM | 1735 | CA | SER | L | 10 | 0.516 | −43.254 | 22.989 | 1.00 | 33.70 | C |
| ATOM | 1736 | CB | SER | L | 10 | −0.855 | −43.484 | 23.615 | 1.00 | 34.08 | C |
| ATOM | 1737 | OG | SER | L | 10 | −1.647 | −42.342 | 23.450 | 1.00 | 36.66 | O |
| ATOM | 1738 | C | SER | L | 10 | 1.269 | −44.560 | 22.988 | 1.00 | 33.06 | C |
| ATOM | 1739 | O | SER | L | 10 | 0.955 | −45.465 | 22.203 | 1.00 | 32.89 | O |
| ATOM | 1740 | N | LEU | L | 11 | 2.251 | −44.651 | 23.875 | 1.00 | 32.53 | N |
| ATOM | 1741 | CA | LEU | L | 11 | 3.084 | −45.829 | 23.966 | 1.00 | 32.97 | C |
| ATOM | 1742 | CB | LEU | L | 11 | 4.468 | −45.516 | 23.377 | 1.00 | 32.69 | C |
| ATOM | 1743 | CG | LEU | L | 11 | 5.524 | −46.602 | 23.244 | 1.00 | 32.59 | C |
| ATOM | 1744 | CD1 | LEU | L | 11 | 5.010 | −47.875 | 22.599 | 1.00 | 34.57 | C |
| ATOM | 1745 | CD2 | LEU | L | 11 | 6.686 | −46.035 | 22.458 | 1.00 | 33.62 | C |
| ATOM | 1746 | C | LEU | L | 11 | 3.183 | −46.327 | 25.417 | 1.00 | 33.22 | C |
| ATOM | 1747 | O | LEU | L | 11 | 3.951 | −45.790 | 26.210 | 1.00 | 32.92 | O |
| ATOM | 1748 | N | PRO | L | 12 | 2.389 | −47.354 | 25.770 | 1.00 | 33.80 | N |
| ATOM | 1749 | CA | PRO | L | 12 | 2.581 | −47.990 | 27.079 | 1.00 | 33.56 | C |
| ATOM | 1750 | CB | PRO | L | 12 | 1.297 | −48.831 | 27.268 | 1.00 | 34.04 | C |
| ATOM | 1751 | CG | PRO | L | 12 | 0.476 | −48.678 | 26.020 | 1.00 | 33.41 | C |
| ATOM | 1752 | CD | PRO | L | 12 | 1.327 | −48.006 | 24.981 | 1.00 | 33.66 | C |
| ATOM | 1753 | C | PRO | L | 12 | 3.796 | −48.898 | 27.036 | 1.00 | 33.35 | C |
| ATOM | 1754 | O | PRO | L | 12 | 3.880 | −49.734 | 26.150 | 1.00 | 33.23 | O |
| ATOM | 1755 | N | VAL | L | 13 | 4.746 | −48.724 | 27.957 | 1.00 | 32.66 | N |
| ATOM | 1756 | CA | VAL | L | 13 | 5.898 | −49.636 | 28.026 | 1.00 | 32.31 | C |
| ATOM | 1757 | CB | VAL | L | 13 | 7.217 | −48.961 | 27.571 | 1.00 | 31.70 | C |
| ATOM | 1758 | CG1 | VAL | L | 13 | 8.347 | −49.990 | 27.481 | 1.00 | 31.47 | C |
| ATOM | 1759 | CG2 | VAL | L | 13 | 7.039 | −48.287 | 26.206 | 1.00 | 33.08 | C |
| ATOM | 1760 | C | VAL | L | 13 | 6.105 | −50.221 | 29.433 | 1.00 | 32.38 | C |
| ATOM | 1761 | O | VAL | L | 13 | 6.109 | −49.494 | 30.420 | 1.00 | 32.04 | O |
| ATOM | 1762 | N | THR | L | 14 | 6.280 | −51.535 | 29.501 | 1.00 | 32.74 | N |
| ATOM | 1763 | CA | THR | L | 14 | 6.664 | −52.234 | 30.733 | 1.00 | 33.37 | C |
| ATOM | 1764 | CB | THR | L | 14 | 6.463 | −53.751 | 30.532 | 1.00 | 33.54 | C |
| ATOM | 1765 | OG1 | THR | L | 14 | 5.056 | −54.015 | 30.429 | 1.00 | 33.57 | O |
| ATOM | 1766 | CG2 | THR | L | 14 | 7.073 | −54.580 | 31.682 | 1.00 | 34.31 | C |
| ATOM | 1767 | C | THR | L | 14 | 8.124 | −51.898 | 31.128 | 1.00 | 33.66 | C |
| ATOM | 1768 | O | THR | L | 14 | 9.033 | −52.064 | 30.304 | 1.00 | 33.30 | O |
| ATOM | 1769 | N | PRO | L | 15 | 8.352 | −51.410 | 32.380 | 1.00 | 33.86 | N |
| ATOM | 1770 | CA | PRO | L | 15 | 9.707 | −51.021 | 32.793 | 1.00 | 34.34 | C |
| ATOM | 1771 | CB | PRO | L | 15 | 9.588 | −50.877 | 34.309 | 1.00 | 34.72 | C |
| ATOM | 1772 | CG | PRO | L | 15 | 8.163 | −50.528 | 34.553 | 1.00 | 34.11 | C |
| ATOM | 1773 | CD | PRO | L | 15 | 7.377 | −51.233 | 33.468 | 1.00 | 34.06 | C |
| ATOM | 1774 | C | PRO | L | 15 | 10.746 | −52.078 | 32.431 | 1.00 | 35.01 | C |
| ATOM | 1775 | O | PRO | L | 15 | 10.501 | −53.275 | 32.579 | 1.00 | 35.41 | O |
| ATOM | 1776 | N | GLY | L | 16 | 11.884 | −51.637 | 31.918 | 1.00 | 35.85 | N |
| ATOM | 1777 | CA | GLY | L | 16 | 12.916 | −52.560 | 31.469 | 1.00 | 36.62 | C |
| ATOM | 1778 | C | GLY | L | 16 | 12.755 | −53.090 | 30.056 | 1.00 | 36.93 | C |
| ATOM | 1779 | O | GLY | L | 16 | 13.668 | −53.735 | 29.552 | 1.00 | 37.17 | O |
| ATOM | 1780 | N | GLU | L | 17 | 11.612 | −52.848 | 29.412 | 1.00 | 37.45 | N |
| ATOM | 1781 | CA | GLU | L | 17 | 11.395 | −53.330 | 28.030 | 1.00 | 38.65 | C |
| ATOM | 1782 | CB | GLU | L | 17 | 9.931 | −53.750 | 27.823 | 1.00 | 39.47 | C |
| ATOM | 1783 | CG | GLU | L | 17 | 9.463 | −54.918 | 28.690 | 1.00 | 42.21 | C |
| ATOM | 1784 | CD | GLU | L | 17 | 10.170 | −56.214 | 28.359 | 1.00 | 45.87 | C |
| ATOM | 1785 | OE1 | GLU | L | 17 | 10.208 | −56.600 | 27.167 | 1.00 | 45.59 | O |
| ATOM | 1786 | OE2 | GLU | L | 17 | 10.698 | −56.844 | 29.298 | 1.00 | 47.85 | O |
| ATOM | 1787 | C | GLU | L | 17 | 11.802 | −52.262 | 26.977 | 1.00 | 38.44 | C |
| ATOM | 1788 | O | GLU | L | 17 | 11.923 | −51.086 | 27.320 | 1.00 | 37.74 | O |
| ATOM | 1789 | N | PRO | L | 18 | 12.021 | −52.670 | 25.698 | 1.00 | 38.96 | N |
| ATOM | 1790 | CA | PRO | L | 18 | 12.364 | −51.685 | 24.645 | 1.00 | 39.30 | C |
| ATOM | 1791 | CB | PRO | L | 18 | 12.804 | −52.552 | 23.449 | 1.00 | 39.41 | C |
| ATOM | 1792 | CG | PRO | L | 18 | 12.803 | −53.966 | 23.928 | 1.00 | 40.08 | C |
| ATOM | 1793 | CD | PRO | L | 18 | 11.959 | −54.042 | 25.160 | 1.00 | 38.99 | C |
| ATOM | 1794 | C | PRO | L | 18 | 11.172 | −50.831 | 24.225 | 1.00 | 38.89 | C |
| ATOM | 1795 | O | PRO | L | 18 | 10.019 | −51.221 | 24.435 | 1.00 | 39.38 | O |
| ATOM | 1796 | N | ALA | L | 19 | 11.450 | −49.669 | 23.649 | 1.00 | 38.82 | N |
| ATOM | 1797 | CA | ALA | L | 19 | 10.408 | −48.837 | 23.037 | 1.00 | 38.14 | C |
| ATOM | 1798 | CB | ALA | L | 19 | 9.987 | −47.745 | 23.985 | 1.00 | 38.17 | C |
| ATOM | 1799 | C | ALA | L | 19 | 10.918 | −48.233 | 21.733 | 1.00 | 38.04 | C |
| ATOM | 1800 | O | ALA | L | 19 | 12.093 | −47.844 | 21.675 | 1.00 | 37.60 | O |
| ATOM | 1801 | N | SER | L | 20 | 10.041 | −48.173 | 20.706 | 1.00 | 37.68 | N |
| ATOM | 1802 | CA | SER | L | 20 | 10.326 | −47.501 | 19.407 | 1.00 | 37.54 | C |
| ATOM | 1803 | CB | SER | L | 20 | 10.343 | −48.488 | 18.228 | 1.00 | 37.63 | C |
| ATOM | 1804 | OG | SER | L | 20 | 11.129 | −49.606 | 18.524 | 1.00 | 41.63 | O |
| ATOM | 1805 | C | SER | L | 20 | 9.307 | −46.429 | 19.070 | 1.00 | 36.01 | C |
| ATOM | 1806 | O | SER | L | 20 | 8.110 | −46.635 | 19.207 | 1.00 | 36.40 | O |
| ATOM | 1807 | N | ILE | L | 21 | 9.790 | −45.293 | 18.599 | 1.00 | 35.07 | N |
| ATOM | 1808 | CA | ILE | L | 21 | 8.927 | −44.200 | 18.173 | 1.00 | 33.92 | C |
| ATOM | 1809 | CB | ILE | L | 21 | 8.992 | −42.994 | 19.134 | 1.00 | 33.54 | C |
| ATOM | 1810 | CG1 | ILE | L | 21 | 8.493 | −43.391 | 20.523 | 1.00 | 33.82 | C |
| ATOM | 1811 | CD1 | ILE | L | 21 | 8.561 | −42.287 | 21.549 | 1.00 | 30.34 | C |
| ATOM | 1812 | CG2 | ILE | L | 21 | 8.137 | −41.834 | 18.615 | 1.00 | 34.95 | C |
| ATOM | 1813 | C | ILE | L | 21 | 9.395 | −43.811 | 16.779 | 1.00 | 33.43 | C |
| ATOM | 1814 | O | ILE | L | 21 | 10.583 | −43.575 | 16.555 | 1.00 | 33.04 | O |
| ATOM | 1815 | N | SER | L | 22 | 8.460 | −43.767 | 15.837 | 1.00 | 33.43 | N |
| ATOM | 1816 | CA | SER | L | 22 | 8.796 | −43.479 | 14.454 | 1.00 | 33.61 | C |
| ATOM | 1817 | CB | SER | L | 22 | 8.158 | −44.525 | 13.542 | 1.00 | 33.62 | C |
| ATOM | 1818 | OG | SER | L | 22 | 7.858 | −43.952 | 12.285 | 1.00 | 37.44 | O |
| ATOM | 1819 | C | SER | L | 22 | 8.367 | −42.050 | 14.116 | 1.00 | 33.45 | C |
| ATOM | 1820 | O | SER | L | 22 | 7.405 | −41.545 | 14.686 | 1.00 | 33.47 | O |
| ATOM | 1821 | N | CYS | L | 23 | 9.118 | −41.386 | 13.237 | 1.00 | 33.33 | N |
| ATOM | 1822 | CA | CYS | L | 23 | 8.775 | −40.049 | 12.749 | 1.00 | 33.14 | C |
| ATOM | 1823 | CB | CYS | L | 23 | 9.672 | −38.988 | 13.416 | 1.00 | 33.62 | C |
| ATOM | 1824 | SG | CYS | L | 23 | 9.484 | −37.267 | 12.889 | 1.00 | 35.65 | S |
| ATOM | 1825 | C | CYS | L | 23 | 8.963 | −40.072 | 11.230 | 1.00 | 33.11 | C |
| ATOM | 1826 | O | CYS | L | 23 | 9.966 | −40.594 | 10.733 | 1.00 | 32.09 | O |
| ATOM | 1827 | N | THR | L | 24 | 7.961 | −39.574 | 10.502 | 1.00 | 32.70 | N |
| ATOM | 1828 | CA | THR | L | 24 | 8.052 | −39.423 | 9.039 | 1.00 | 33.01 | C |
| ATOM | 1829 | CB | THR | L | 24 | 6.986 | −40.272 | 8.292 | 1.00 | 33.04 | C |
| ATOM | 1830 | OG1 | THR | L | 24 | 7.110 | −41.637 | 8.686 | 1.00 | 33.67 | O |
| ATOM | 1831 | CG2 | THR | L | 24 | 7.209 | −40.209 | 6.765 | 1.00 | 33.82 | C |
| ATOM | 1832 | C | THR | L | 24 | 7.899 | −37.946 | 8.666 | 1.00 | 32.12 | C |
| ATOM | 1833 | O | THR | L | 24 | 7.033 | −37.266 | 9.181 | 1.00 | 32.21 | O |
| ATOM | 1834 | N | SER | L | 25 | 8.756 | −37.440 | 7.797 | 1.00 | 31.78 | N |
| ATOM | 1835 | CA | SER | L | 25 | 8.585 | −36.065 | 7.331 | 1.00 | 31.83 | C |
| ATOM | 1836 | CB | SER | L | 25 | 9.937 | −35.331 | 7.272 | 1.00 | 30.91 | C |
| ATOM | 1837 | OG | SER | L | 25 | 10.837 | −36.020 | 6.443 | 1.00 | 32.31 | O |
| ATOM | 1838 | C | SER | L | 25 | 7.906 | −36.039 | 5.955 | 1.00 | 31.46 | C |
| ATOM | 1839 | O | SER | L | 25 | 8.053 | −36.983 | 5.166 | 1.00 | 31.11 | O |
| ATOM | 1840 | N | GLY | L | 26 | 7.188 | −34.955 | 5.677 | 1.00 | 31.31 | N |
| ATOM | 1841 | CA | GLY | L | 26 | 6.500 | −34.782 | 4.370 | 1.00 | 32.48 | C |
| ATOM | 1842 | C | GLY | L | 26 | 7.446 | −34.746 | 3.172 | 1.00 | 32.57 | C |
| ATOM | 1843 | O | GLY | L | 26 | 7.081 | −35.160 | 2.077 | 1.00 | 33.31 | O |
| ATOM | 1844 | N | GLN | L | 27 | 8.665 | −34.257 | 3.383 | 1.00 | 31.85 | N |
| ATOM | 1845 | CA | GLN | L | 27 | 9.704 | −34.304 | 2.367 | 1.00 | 32.09 | C |
| ATOM | 1846 | CB | GLN | L | 27 | 9.704 | −33.020 | 1.532 | 1.00 | 32.37 | C |
| ATOM | 1847 | CG | GLN | L | 27 | 10.066 | −31.768 | 2.264 | 1.00 | 32.44 | C |
| ATOM | 1848 | CD | GLN | L | 27 | 9.846 | −30.538 | 1.407 | 1.00 | 36.27 | C |
| ATOM | 1849 | OE1 | GLN | L | 27 | 8.791 | −29.912 | 1.467 | 1.00 | 37.74 | O |
| ATOM | 1850 | NE2 | GLN | L | 27 | 10.831 | −30.205 | 0.579 | 1.00 | 36.44 | N |
| ATOM | 1851 | C | GLN | L | 27 | 11.083 | −34.491 | 2.988 | 1.00 | 31.56 | C |
| ATOM | 1852 | O | GLN | L | 27 | 11.244 | −34.360 | 4.187 | 1.00 | 31.03 | O |
| ATOM | 1853 | N | SER | L | 27A | 12.080 | −34.738 | 2.151 | 1.00 | 30.76 | N |
| ATOM | 1854 | CA | SER | L | 27A | 13.416 | −35.025 | 2.634 | 1.00 | 30.71 | C |
| ATOM | 1855 | CB | SER | L | 27A | 14.373 | −35.225 | 1.477 | 1.00 | 30.57 | C |
| ATOM | 1856 | OG | SER | L | 27A | 15.650 | −35.393 | 2.026 | 1.00 | 31.20 | O |
| ATOM | 1857 | C | SER | L | 27A | 13.980 | −33.945 | 3.557 | 1.00 | 30.01 | C |
| ATOM | 1858 | O | SER | L | 27A | 13.798 | −32.760 | 3.327 | 1.00 | 29.82 | O |
| ATOM | 1859 | N | LEU | L | 27B | 14.685 | −34.384 | 4.596 | 1.00 | 29.84 | N |
| ATOM | 1860 | CA | LEU | L | 27B | 15.312 | −33.476 | 5.554 | 1.00 | 29.66 | C |
| ATOM | 1861 | CB | LEU | L | 27B | 15.030 | −33.939 | 7.000 | 1.00 | 28.88 | C |
| ATOM | 1862 | CG | LEU | L | 27B | 13.554 | −34.017 | 7.400 | 1.00 | 28.36 | C |
| ATOM | 1863 | CD1 | LEU | L | 27B | 13.410 | −34.433 | 8.872 | 1.00 | 29.28 | C |
| ATOM | 1864 | CD2 | LEU | L | 27B | 12.792 | −32.712 | 7.111 | 1.00 | 25.60 | C |
| ATOM | 1865 | C | LEU | L | 27B | 16.809 | −33.322 | 5.301 | 1.00 | 30.08 | C |
| ATOM | 1866 | O | LEU | L | 27B | 17.540 | −32.717 | 6.105 | 1.00 | 29.55 | O |
| ATOM | 1867 | N | VAL | L | 27C | 17.267 | −33.875 | 4.181 | 1.00 | 31.03 | N |
| ATOM | 1868 | CA | VAL | L | 27C | 18.632 | −33.628 | 3.708 | 1.00 | 31.53 | C |
| ATOM | 1869 | CB | VAL | L | 27C | 19.032 | −34.587 | 2.554 | 1.00 | 31.81 | C |
| ATOM | 1870 | CG1 | VAL | L | 27C | 20.435 | −34.268 | 2.048 | 1.00 | 31.84 | C |
| ATOM | 1871 | CG2 | VAL | L | 27C | 18.960 | −36.036 | 3.031 | 1.00 | 31.12 | C |
| ATOM | 1872 | C | VAL | L | 27C | 18.728 | −32.171 | 3.277 | 1.00 | 32.14 | C |
| ATOM | 1873 | O | VAL | L | 27C | 17.956 | −31.715 | 2.419 | 1.00 | 32.37 | O |
| ATOM | 1874 | N | HIS | L | 27D | 19.651 | −31.444 | 3.906 | 1.00 | 32.15 | N |
| ATOM | 1875 | CA | HIS | L | 27D | 19.897 | −30.043 | 3.621 | 1.00 | 33.09 | C |
| ATOM | 1876 | CB | HIS | L | 27D | 20.583 | −29.413 | 4.842 | 1.00 | 32.97 | C |
| ATOM | 1877 | CG | HIS | L | 27D | 20.557 | −27.913 | 4.859 | 1.00 | 33.30 | C |
| ATOM | 1878 | ND1 | HIS | L | 27D | 21.637 | −27.147 | 4.483 | 1.00 | 32.15 | N |
| ATOM | 1879 | CE1 | HIS | L | 27D | 21.334 | −25.869 | 4.606 | 1.00 | 32.91 | C |
| ATOM | 1880 | NE2 | HIS | L | 27D | 20.096 | −25.779 | 5.059 | 1.00 | 35.15 | N |
| ATOM | 1881 | CD2 | HIS | L | 27D | 19.587 | −27.042 | 5.223 | 1.00 | 32.72 | C |
| ATOM | 1882 | C | HIS | L | 27D | 20.831 | −29.938 | 2.423 | 1.00 | 34.44 | C |
| ATOM | 1883 | O | HIS | L | 27D | 21.548 | −30.886 | 2.124 | 1.00 | 34.63 | O |
| ATOM | 1884 | N | ILE | L | 27E | 20.827 | −28.780 | 1.760 | 1.00 | 35.64 | N |
| ATOM | 1885 | CA | ILE | L | 27E | 21.800 | −28.456 | 0.705 | 1.00 | 37.21 | C |
| ATOM | 1886 | CB | ILE | L | 27E | 21.484 | −27.074 | 0.045 | 1.00 | 37.50 | C |
| ATOM | 1887 | CG1 | ILE | L | 27E | 22.502 | −26.707 | −1.034 | 1.00 | 40.26 | C |
| ATOM | 1888 | CD1 | ILE | L | 27E | 22.266 | −25.281 | −1.651 | 1.00 | 41.75 | C |
| ATOM | 1889 | CG2 | ILE | L | 27E | 21.369 | −25.947 | 1.081 | 1.00 | 38.50 | C |
| ATOM | 1890 | C | ILE | L | 27E | 23.282 | −28.596 | 1.143 | 1.00 | 36.84 | C |
| ATOM | 1891 | O | ILE | L | 27E | 24.142 | −28.875 | 0.308 | 1.00 | 37.63 | O |
| ATOM | 1892 | N | ASN | L | 28 | 23.573 | −28.439 | 2.444 | 1.00 | 35.92 | N |
| ATOM | 1893 | CA | ASN | L | 28 | 24.931 | −28.654 | 2.970 | 1.00 | 34.08 | C |
| ATOM | 1894 | CB | ASN | L | 28 | 25.201 | −27.787 | 4.215 | 1.00 | 34.49 | C |
| ATOM | 1895 | CG | ASN | L | 28 | 24.419 | −28.242 | 5.467 | 1.00 | 32.49 | C |
| ATOM | 1896 | OD1 | ASN | L | 28 | 23.978 | −29.397 | 5.594 | 1.00 | 29.29 | O |
| ATOM | 1897 | ND2 | ASN | L | 28 | 24.268 | −27.324 | 6.401 | 1.00 | 31.21 | N |
| ATOM | 1898 | C | ASN | L | 28 | 25.254 | −30.111 | 3.273 | 1.00 | 33.69 | C |
| ATOM | 1899 | O | ASN | L | 28 | 26.342 | −30.423 | 3.766 | 1.00 | 33.97 | O |
| ATOM | 1900 | N | GLY | L | 29 | 24.300 | −30.999 | 3.016 | 1.00 | 32.89 | N |
| ATOM | 1901 | CA | GLY | L | 29 | 24.530 | −32.421 | 3.195 | 1.00 | 32.32 | C |
| ATOM | 1902 | C | GLY | L | 29 | 24.076 | −33.000 | 4.521 | 1.00 | 32.25 | C |
| ATOM | 1903 | O | GLY | L | 29 | 24.041 | −34.210 | 4.664 | 1.00 | 31.39 | O |
| ATOM | 1904 | N | ASN | L | 30 | 23.743 | −32.144 | 5.496 | 1.00 | 32.60 | N |
| ATOM | 1905 | CA | ASN | L | 30 | 23.302 | −32.623 | 6.823 | 1.00 | 32.25 | C |
| ATOM | 1906 | CB | ASN | L | 30 | 23.573 | −31.594 | 7.922 | 1.00 | 32.51 | C |
| ATOM | 1907 | CG | ASN | L | 30 | 25.030 | −31.533 | 8.330 | 1.00 | 31.41 | C |
| ATOM | 1908 | OD1 | ASN | L | 30 | 25.622 | −32.545 | 8.658 | 1.00 | 36.66 | O |
| ATOM | 1909 | ND2 | ASN | L | 30 | 25.599 | −30.365 | 8.314 | 1.00 | 30.79 | N |
| ATOM | 1910 | C | ASN | L | 30 | 21.819 | −32.932 | 6.842 | 1.00 | 32.27 | C |
| ATOM | 1911 | O | ASN | L | 30 | 21.040 | −32.319 | 6.102 | 1.00 | 31.52 | O |
| ATOM | 1912 | N | THR | L | 31 | 21.444 | −33.870 | 7.708 | 1.00 | 31.92 | N |
| ATOM | 1913 | CA | THR | L | 31 | 20.044 | −34.175 | 7.975 | 1.00 | 31.86 | C |
| ATOM | 1914 | CB | THR | L | 31 | 19.768 | −35.669 | 7.786 | 1.00 | 31.66 | C |
| ATOM | 1915 | OG1 | THR | L | 31 | 20.009 | −35.991 | 6.417 | 1.00 | 32.82 | O |
| ATOM | 1916 | CG2 | THR | L | 31 | 18.307 | −36.006 | 8.134 | 1.00 | 31.09 | C |
| ATOM | 1917 | C | THR | L | 31 | 19.625 | −33.694 | 9.370 | 1.00 | 31.58 | C |
| ATOM | 1918 | O | THR | L | 31 | 19.988 | −34.295 | 10.392 | 1.00 | 31.49 | O |
| ATOM | 1919 | N | TYR | L | 32 | 18.861 | −32.609 | 9.393 | 1.00 | 30.64 | N |
| ATOM | 1920 | CA | TYR | L | 32 | 18.490 | −31.944 | 10.641 | 1.00 | 30.81 | C |
| ATOM | 1921 | CB | TYR | L | 32 | 18.385 | −30.441 | 10.427 | 1.00 | 30.43 | C |
| ATOM | 1922 | CG | TYR | L | 32 | 19.703 | −29.822 | 10.045 | 1.00 | 33.12 | C |
| ATOM | 1923 | CD1 | TYR | L | 32 | 20.675 | −29.550 | 11.010 | 1.00 | 32.03 | C |
| ATOM | 1924 | CE1 | TYR | L | 32 | 21.913 | −28.992 | 10.650 | 1.00 | 33.00 | C |
| ATOM | 1925 | CZ | TYR | L | 32 | 22.163 | −28.692 | 9.309 | 1.00 | 35.77 | C |
| ATOM | 1926 | OH | TYR | L | 32 | 23.351 | −28.123 | 8.922 | 1.00 | 36.97 | O |
| ATOM | 1927 | CE2 | TYR | L | 32 | 21.209 | −28.958 | 8.334 | 1.00 | 36.11 | C |
| ATOM | 1928 | CD2 | TYR | L | 32 | 19.987 | −29.517 | 8.701 | 1.00 | 33.41 | C |
| ATOM | 1929 | C | TYR | L | 32 | 17.193 | −32.470 | 11.240 | 1.00 | 30.07 | C |
| ATOM | 1930 | O | TYR | L | 32 | 16.181 | −31.769 | 11.298 | 1.00 | 29.35 | O |
| ATOM | 1931 | N | LEU | L | 33 | 17.260 | −33.713 | 11.689 | 1.00 | 29.77 | N |
| ATOM | 1932 | CA | LEU | L | 33 | 16.170 | −34.345 | 12.397 | 1.00 | 29.51 | C |
| ATOM | 1933 | CB | LEU | L | 33 | 15.790 | −35.670 | 11.725 | 1.00 | 28.34 | C |
| ATOM | 1934 | CG | LEU | L | 33 | 14.526 | −36.400 | 12.206 | 1.00 | 28.23 | C |
| ATOM | 1935 | CD1 | LEU | L | 33 | 13.308 | −35.488 | 12.335 | 1.00 | 26.04 | C |
| ATOM | 1936 | CD2 | LEU | L | 33 | 14.766 | −37.184 | 13.494 | 1.00 | 30.86 | C |
| ATOM | 1937 | C | LEU | L | 33 | 16.649 | −34.588 | 13.824 | 1.00 | 28.96 | C |
| ATOM | 1938 | O | LEU | L | 33 | 17.652 | −35.269 | 14.036 | 1.00 | 29.31 | O |
| ATOM | 1939 | N | HIS | L | 34 | 15.932 | −34.027 | 14.788 | 1.00 | 29.09 | N |
| ATOM | 1940 | CA | HIS | L | 34 | 16.297 | −34.187 | 16.195 | 1.00 | 29.08 | C |
| ATOM | 1941 | CB | HIS | L | 34 | 16.708 | −32.844 | 16.767 | 1.00 | 29.57 | C |
| ATOM | 1942 | CG | HIS | L | 34 | 17.426 | −31.970 | 15.785 | 1.00 | 28.94 | C |
| ATOM | 1943 | ND1 | HIS | L | 34 | 18.678 | −32.273 | 15.296 | 1.00 | 31.21 | N |
| ATOM | 1944 | CE1 | HIS | L | 34 | 19.067 | −31.322 | 14.469 | 1.00 | 30.55 | C |
| ATOM | 1945 | NE2 | HIS | L | 34 | 18.102 | −30.424 | 14.390 | 1.00 | 32.25 | N |
| ATOM | 1946 | CD2 | HIS | L | 34 | 17.064 | −30.810 | 15.200 | 1.00 | 28.20 | C |
| ATOM | 1947 | C | HIS | L | 34 | 15.150 | −34.771 | 17.022 | 1.00 | 29.52 | C |
| ATOM | 1948 | O | HIS | L | 34 | 13.977 | −34.661 | 16.633 | 1.00 | 29.59 | O |
| ATOM | 1949 | N | TRP | L | 35 | 15.498 | −35.410 | 18.141 | 1.00 | 28.60 | N |
| ATOM | 1950 | CA | TRP | L | 35 | 14.511 | −35.983 | 19.036 | 1.00 | 28.36 | C |
| ATOM | 1951 | CB | TRP | L | 35 | 14.724 | −37.483 | 19.178 | 1.00 | 28.36 | C |
| ATOM | 1952 | CG | TRP | L | 35 | 14.381 | −38.302 | 17.979 | 1.00 | 29.00 | C |
| ATOM | 1953 | CD1 | TRP | L | 35 | 15.246 | −38.775 | 17.033 | 1.00 | 28.21 | C |
| ATOM | 1954 | NE1 | TRP | L | 35 | 14.573 | −39.526 | 16.107 | 1.00 | 28.16 | N |
| ATOM | 1955 | CE2 | TRP | L | 35 | 13.240 | −39.554 | 16.438 | 1.00 | 29.78 | C |
| ATOM | 1956 | CD2 | TRP | L | 35 | 13.081 | −38.794 | 17.616 | 1.00 | 29.85 | C |
| ATOM | 1957 | CE3 | TRP | L | 35 | 11.804 | −38.678 | 18.179 | 1.00 | 28.52 | C |
| ATOM | 1958 | CZ3 | TRP | L | 35 | 10.730 | −39.317 | 17.538 | 1.00 | 29.23 | C |
| ATOM | 1959 | CH2 | TRP | L | 35 | 10.924 | −40.061 | 16.374 | 1.00 | 28.44 | C |
| ATOM | 1960 | CZ2 | TRP | L | 35 | 12.166 | −40.191 | 15.805 | 1.00 | 27.92 | C |
| ATOM | 1961 | C | TRP | L | 35 | 14.603 | −35.355 | 20.417 | 1.00 | 29.09 | C |
| ATOM | 1962 | O | TRP | L | 35 | 15.712 | −35.223 | 20.992 | 1.00 | 28.25 | O |
| ATOM | 1963 | N | TYR | L | 36 | 13.438 | −34.994 | 20.947 | 1.00 | 28.83 | N |
| ATOM | 1964 | CA | TYR | L | 36 | 13.309 | −34.440 | 22.289 | 1.00 | 29.55 | C |
| ATOM | 1965 | CB | TYR | L | 36 | 12.721 | −33.024 | 22.212 | 1.00 | 28.88 | C |
| ATOM | 1966 | CG | TYR | L | 36 | 13.687 | −32.032 | 21.619 | 1.00 | 30.19 | C |
| ATOM | 1967 | CD1 | TYR | L | 36 | 14.405 | −31.168 | 22.438 | 1.00 | 29.16 | C |
| ATOM | 1968 | CE1 | TYR | L | 36 | 15.296 | −30.244 | 21.903 | 1.00 | 30.26 | C |
| ATOM | 1969 | CZ | TYR | L | 36 | 15.511 | −30.207 | 20.533 | 1.00 | 29.94 | C |
| ATOM | 1970 | OH | TYR | L | 36 | 16.419 | −29.290 | 20.018 | 1.00 | 32.03 | O |
| ATOM | 1971 | CE2 | TYR | L | 36 | 14.821 | −31.073 | 19.688 | 1.00 | 28.95 | C |
| ATOM | 1972 | CD2 | TYR | L | 36 | 13.914 | −31.981 | 20.230 | 1.00 | 29.59 | C |
| ATOM | 1973 | C | TYR | L | 36 | 12.425 | −35.285 | 23.213 | 1.00 | 29.74 | C |
| ATOM | 1974 | O | TYR | L | 36 | 11.522 | −35.994 | 22.749 | 1.00 | 29.49 | O |
| ATOM | 1975 | N | LEU | L | 37 | 12.670 | −35.158 | 24.519 | 1.00 | 30.19 | N |
| ATOM | 1976 | CA | LEU | L | 37 | 11.766 | −35.652 | 25.560 | 1.00 | 30.97 | C |
| ATOM | 1977 | CB | LEU | L | 37 | 12.422 | −36.747 | 26.406 | 1.00 | 31.16 | C |
| ATOM | 1978 | CG | LEU | L | 37 | 11.827 | −37.139 | 27.782 | 1.00 | 30.38 | C |
| ATOM | 1979 | CD1 | LEU | L | 37 | 10.378 | −37.627 | 27.715 | 1.00 | 27.73 | C |
| ATOM | 1980 | CD2 | LEU | L | 37 | 12.720 | −38.214 | 28.406 | 1.00 | 32.29 | C |
| ATOM | 1981 | C | LEU | L | 37 | 11.324 | −34.494 | 26.436 | 1.00 | 31.84 | C |
| ATOM | 1982 | O | LEU | L | 37 | 12.146 | −33.714 | 26.936 | 1.00 | 32.02 | O |
| ATOM | 1983 | N | GLN | L | 38 | 10.017 | −34.347 | 26.578 | 1.00 | 33.19 | N |
| ATOM | 1984 | CA | GLN | L | 38 | 9.467 | −33.365 | 27.485 | 1.00 | 34.62 | C |
| ATOM | 1985 | CB | GLN | L | 38 | 8.456 | −32.456 | 26.798 | 1.00 | 34.64 | C |
| ATOM | 1986 | CG | GLN | L | 38 | 7.901 | −31.388 | 27.755 | 1.00 | 34.67 | C |
| ATOM | 1987 | CD | GLN | L | 38 | 6.865 | −30.480 | 27.119 | 1.00 | 34.56 | C |
| ATOM | 1988 | OE1 | GLN | L | 38 | 6.142 | −30.879 | 26.205 | 1.00 | 34.08 | O |
| ATOM | 1989 | NE2 | GLN | L | 38 | 6.792 | −29.243 | 27.603 | 1.00 | 34.45 | N |
| ATOM | 1990 | C | GLN | L | 38 | 8.818 | −34.092 | 28.662 | 1.00 | 35.90 | C |
| ATOM | 1991 | O | GLN | L | 38 | 7.649 | −34.476 | 28.587 | 1.00 | 36.17 | O |
| ATOM | 1992 | N | LYS | L | 39 | 9.599 | −34.283 | 29.723 | 1.00 | 36.60 | N |
| ATOM | 1993 | CA | LYS | L | 39 | 9.110 | −34.807 | 31.003 | 1.00 | 38.36 | C |
| ATOM | 1994 | CB | LYS | L | 39 | 10.269 | −34.944 | 31.998 | 1.00 | 38.25 | C |
| ATOM | 1995 | CG | LYS | L | 39 | 11.434 | −35.733 | 31.453 | 1.00 | 39.46 | C |
| ATOM | 1996 | CD | LYS | L | 39 | 12.565 | −35.858 | 32.469 | 1.00 | 44.11 | C |
| ATOM | 1997 | CE | LYS | L | 39 | 13.709 | −36.679 | 31.886 | 1.00 | 44.37 | C |
| ATOM | 1998 | NZ | LYS | L | 39 | 14.820 | −36.901 | 32.853 | 1.00 | 46.80 | N |
| ATOM | 1999 | C | LYS | L | 39 | 8.006 | −33.919 | 31.581 | 1.00 | 38.97 | C |
| ATOM | 2000 | O | LYS | L | 39 | 7.950 | −32.735 | 31.260 | 1.00 | 38.86 | O |
| ATOM | 2001 | N | PRO | L | 40 | 7.109 | −34.495 | 32.419 | 1.00 | 40.07 | N |
| ATOM | 2002 | CA | PRO | L | 40 | 5.929 | −33.754 | 32.923 | 1.00 | 40.66 | C |
| ATOM | 2003 | CB | PRO | L | 40 | 5.278 | −34.740 | 33.909 | 1.00 | 40.98 | C |
| ATOM | 2004 | CG | PRO | L | 40 | 5.728 | −36.107 | 33.435 | 1.00 | 40.82 | C |
| ATOM | 2005 | CD | PRO | L | 40 | 7.132 | −35.897 | 32.897 | 1.00 | 40.23 | C |
| ATOM | 2006 | C | PRO | L | 40 | 6.273 | −32.432 | 33.633 | 1.00 | 40.70 | C |
| ATOM | 2007 | O | PRO | L | 40 | 7.155 | −32.400 | 34.469 | 1.00 | 40.69 | O |
| ATOM | 2008 | N | GLY | L | 41 | 5.582 | −31.353 | 33.281 | 1.00 | 40.97 | N |
| ATOM | 2009 | CA | GLY | L | 41 | 5.868 | −30.035 | 33.847 | 1.00 | 41.26 | C |
| ATOM | 2010 | C | GLY | L | 41 | 7.141 | −29.343 | 33.341 | 1.00 | 41.58 | C |
| ATOM | 2011 | O | GLY | L | 41 | 7.303 | −28.137 | 33.528 | 1.00 | 42.10 | O |
| ATOM | 2012 | N | GLN | L | 42 | 8.038 | −30.076 | 32.687 | 1.00 | 40.77 | N |
| ATOM | 2013 | CA | GLN | L | 42 | 9.298 | −29.479 | 32.213 | 1.00 | 40.65 | C |
| ATOM | 2014 | CB | GLN | L | 42 | 10.457 | −30.445 | 32.443 | 1.00 | 40.73 | C |
| ATOM | 2015 | CG | GLN | L | 42 | 10.925 | −30.481 | 33.884 | 1.00 | 44.79 | C |
| ATOM | 2016 | CD | GLN | L | 42 | 11.647 | −31.751 | 34.192 | 1.00 | 49.37 | C |
| ATOM | 2017 | OE1 | GLN | L | 42 | 12.588 | −32.126 | 33.492 | 1.00 | 50.58 | O |
| ATOM | 2018 | NE2 | GLN | L | 42 | 11.207 | −32.446 | 35.241 | 1.00 | 52.23 | N |
| ATOM | 2019 | C | GLN | L | 42 | 9.286 | −28.976 | 30.754 | 1.00 | 39.35 | C |
| ATOM | 2020 | O | GLN | L | 42 | 8.362 | −29.254 | 29.985 | 1.00 | 39.29 | O |
| ATOM | 2021 | N | SER | L | 43 | 10.313 | −28.212 | 30.388 | 1.00 | 37.95 | N |
| ATOM | 2022 | CA | SER | L | 43 | 10.536 | −27.859 | 28.988 | 1.00 | 36.93 | C |
| ATOM | 2023 | CB | SER | L | 43 | 11.475 | −26.658 | 28.862 | 1.00 | 36.97 | C |
| ATOM | 2024 | OG | SER | L | 43 | 12.742 | −26.955 | 29.414 | 1.00 | 36.39 | O |
| ATOM | 2025 | C | SER | L | 43 | 11.110 | −29.073 | 28.270 | 1.00 | 35.91 | C |
| ATOM | 2026 | O | SER | L | 43 | 11.618 | −29.998 | 28.928 | 1.00 | 35.82 | O |
| ATOM | 2027 | N | PRO | L | 44 | 11.022 | −29.103 | 26.921 | 1.00 | 34.88 | N |
| ATOM | 2028 | CA | PRO | L | 44 | 11.646 | −30.230 | 26.204 | 1.00 | 34.05 | C |
| ATOM | 2029 | CB | PRO | L | 44 | 11.322 | −29.947 | 24.726 | 1.00 | 33.97 | C |
| ATOM | 2030 | CG | PRO | L | 44 | 10.188 | −29.013 | 24.743 | 1.00 | 33.95 | C |
| ATOM | 2031 | CD | PRO | L | 44 | 10.302 | −28.203 | 26.007 | 1.00 | 34.59 | C |
| ATOM | 2032 | C | PRO | L | 44 | 13.151 | −30.276 | 26.394 | 1.00 | 32.96 | C |
| ATOM | 2033 | O | PRO | L | 44 | 13.797 | −29.252 | 26.502 | 1.00 | 32.57 | O |
| ATOM | 2034 | N | LYS | L | 45 | 13.700 | −31.476 | 26.434 | 1.00 | 33.29 | N |
| ATOM | 2035 | CA | LYS | L | 45 | 15.131 | −31.646 | 26.460 | 1.00 | 33.56 | C |
| ATOM | 2036 | CB | LYS | L | 45 | 15.571 | −32.276 | 27.791 | 1.00 | 34.11 | C |
| ATOM | 2037 | CG | LYS | L | 45 | 15.814 | −31.254 | 28.905 | 1.00 | 37.50 | C |
| ATOM | 2038 | CD | LYS | L | 45 | 16.671 | −31.881 | 30.016 | 1.00 | 41.32 | C |
| ATOM | 2039 | CE | LYS | L | 45 | 17.601 | −30.845 | 30.672 | 1.00 | 43.99 | C |
| ATOM | 2040 | NZ | LYS | L | 45 | 18.874 | −31.459 | 31.174 | 1.00 | 45.07 | N |
| ATOM | 2041 | C | LYS | L | 45 | 15.610 | −32.512 | 25.286 | 1.00 | 32.71 | C |
| ATOM | 2042 | O | LYS | L | 45 | 15.002 | −33.542 | 24.967 | 1.00 | 31.41 | O |
| ATOM | 2043 | N | LEU | L | 46 | 16.737 | −32.107 | 24.704 | 1.00 | 32.04 | N |
| ATOM | 2044 | CA | LEU | L | 46 | 17.352 | −32.784 | 23.555 | 1.00 | 31.41 | C |
| ATOM | 2045 | CB | LEU | L | 46 | 18.368 | −31.843 | 22.907 | 1.00 | 31.24 | C |
| ATOM | 2046 | CG | LEU | L | 46 | 19.254 | −32.289 | 21.746 | 1.00 | 31.03 | C |
| ATOM | 2047 | CD1 | LEU | L | 46 | 18.404 | −32.659 | 20.488 | 1.00 | 28.52 | C |
| ATOM | 2048 | CD2 | LEU | L | 46 | 20.272 | −31.168 | 21.444 | 1.00 | 30.28 | C |
| ATOM | 2049 | C | LEU | L | 46 | 17.987 | −34.129 | 23.886 | 1.00 | 31.74 | C |
| ATOM | 2050 | O | LEU | L | 46 | 18.796 | −34.254 | 24.821 | 1.00 | 32.08 | O |
| ATOM | 2051 | N | LEU | L | 47 | 17.610 | −35.138 | 23.115 | 1.00 | 31.03 | N |
| ATOM | 2052 | CA | LEU | L | 47 | 18.118 | −36.478 | 23.283 | 1.00 | 31.41 | C |
| ATOM | 2053 | CB | LEU | L | 47 | 16.967 | −37.496 | 23.205 | 1.00 | 31.43 | C |
| ATOM | 2054 | CG | LEU | L | 47 | 15.829 | −37.292 | 24.224 | 1.00 | 31.94 | C |
| ATOM | 2055 | CD1 | LEU | L | 47 | 14.662 | −38.214 | 23.934 | 1.00 | 32.31 | C |
| ATOM | 2056 | CD2 | LEU | L | 47 | 16.356 | −37.493 | 25.663 | 1.00 | 30.83 | C |
| ATOM | 2057 | C | LEU | L | 47 | 19.146 | −36.828 | 22.222 | 1.00 | 31.88 | C |
| ATOM | 2058 | O | LEU | L | 47 | 20.224 | −37.367 | 22.532 | 1.00 | 31.96 | O |
| ATOM | 2059 | N | ILE | L | 48 | 18.792 | −36.534 | 20.971 | 1.00 | 32.15 | N |
| ATOM | 2060 | CA | ILE | L | 48 | 19.522 | −37.010 | 19.792 | 1.00 | 31.94 | C |
| ATOM | 2061 | CB | ILE | L | 48 | 18.937 | −38.325 | 19.276 | 1.00 | 32.49 | C |
| ATOM | 2062 | CG1 | ILE | L | 48 | 19.312 | −39.462 | 20.228 | 1.00 | 32.61 | C |
| ATOM | 2063 | CD1 | ILE | L | 48 | 18.585 | −40.689 | 19.925 | 1.00 | 32.88 | C |
| ATOM | 2064 | CG2 | ILE | L | 48 | 19.478 | −38.704 | 17.872 | 1.00 | 32.40 | C |
| ATOM | 2065 | C | ILE | L | 48 | 19.446 | −35.908 | 18.741 | 1.00 | 32.19 | C |
| ATOM | 2066 | O | ILE | L | 48 | 18.363 | −35.364 | 18.487 | 1.00 | 31.99 | O |
| ATOM | 2067 | N | TYR | L | 49 | 20.608 | −35.520 | 18.205 | 1.00 | 31.72 | N |
| ATOM | 2068 | CA | TYR | L | 49 | 20.671 | −34.508 | 17.143 | 1.00 | 31.21 | C |
| ATOM | 2069 | CB | TYR | L | 49 | 21.485 | −33.273 | 17.565 | 1.00 | 31.32 | C |
| ATOM | 2070 | CG | TYR | L | 49 | 22.974 | −33.498 | 17.709 | 1.00 | 31.84 | C |
| ATOM | 2071 | CD1 | TYR | L | 49 | 23.804 | −33.429 | 16.602 | 1.00 | 33.16 | C |
| ATOM | 2072 | CE1 | TYR | L | 49 | 25.178 | −33.610 | 16.706 | 1.00 | 35.08 | C |
| ATOM | 2073 | CZ | TYR | L | 49 | 25.748 | −33.887 | 17.939 | 1.00 | 35.37 | C |
| ATOM | 2074 | OH | TYR | L | 49 | 27.103 | −34.079 | 17.990 | 1.00 | 34.54 | O |
| ATOM | 2075 | CE2 | TYR | L | 49 | 24.948 | −33.973 | 19.083 | 1.00 | 36.04 | C |
| ATOM | 2076 | CD2 | TYR | L | 49 | 23.551 | −33.760 | 18.957 | 1.00 | 34.76 | C |
| ATOM | 2077 | C | TYR | L | 49 | 21.171 | −35.123 | 15.847 | 1.00 | 30.54 | C |
| ATOM | 2078 | O | TYR | L | 49 | 21.908 | −36.114 | 15.865 | 1.00 | 30.37 | O |
| ATOM | 2079 | N | LYS | L | 50 | 20.717 | −34.544 | 14.735 | 1.00 | 30.95 | N |
| ATOM | 2080 | CA | LYS | L | 50 | 21.010 | −35.014 | 13.375 | 1.00 | 31.08 | C |
| ATOM | 2081 | CB | LYS | L | 50 | 22.370 | −34.492 | 12.863 | 1.00 | 31.14 | C |
| ATOM | 2082 | CG | LYS | L | 50 | 22.375 | −32.992 | 12.536 | 1.00 | 31.61 | C |
| ATOM | 2083 | CD | LYS | L | 50 | 23.678 | −32.523 | 11.848 | 1.00 | 33.71 | C |
| ATOM | 2084 | CE | LYS | L | 50 | 24.932 | −32.721 | 12.722 | 1.00 | 33.84 | C |
| ATOM | 2085 | NZ | LYS | L | 50 | 26.178 | −32.308 | 11.992 | 1.00 | 36.75 | N |
| ATOM | 2086 | C | LYS | L | 50 | 20.918 | −36.511 | 13.244 | 1.00 | 31.29 | C |
| ATOM | 2087 | O | LYS | L | 50 | 21.919 | −37.189 | 12.973 | 1.00 | 31.56 | O |
| ATOM | 2088 | N | VAL | L | 51 | 19.706 | −37.019 | 13.469 | 1.00 | 31.73 | N |
| ATOM | 2089 | CA | VAL | L | 51 | 19.367 | −38.430 | 13.298 | 1.00 | 31.37 | C |
| ATOM | 2090 | CB | VAL | L | 51 | 19.773 | −38.976 | 11.882 | 1.00 | 31.72 | C |
| ATOM | 2091 | CG1 | VAL | L | 51 | 19.385 | −40.428 | 11.738 | 1.00 | 28.43 | C |
| ATOM | 2092 | CG2 | VAL | L | 51 | 19.112 | −38.154 | 10.782 | 1.00 | 30.71 | C |
| ATOM | 2093 | C | VAL | L | 51 | 19.849 | −39.404 | 14.407 | 1.00 | 32.53 | C |
| ATOM | 2094 | O | VAL | L | 51 | 19.051 | −40.190 | 14.925 | 1.00 | 32.09 | O |
| ATOM | 2095 | N | SER | L | 52 | 21.143 | −39.381 | 14.730 | 1.00 | 33.27 | N |
| ATOM | 2096 | CA | SER | L | 52 | 21.753 | −40.478 | 15.484 | 1.00 | 34.16 | C |
| ATOM | 2097 | CB | SER | L | 52 | 22.298 | −41.500 | 14.505 | 1.00 | 34.41 | C |
| ATOM | 2098 | OG | SER | L | 52 | 23.273 | −40.857 | 13.713 | 1.00 | 37.35 | O |
| ATOM | 2099 | C | SER | L | 52 | 22.877 | −40.074 | 16.455 | 1.00 | 34.11 | C |
| ATOM | 2100 | O | SER | L | 52 | 23.565 | −40.938 | 17.000 | 1.00 | 34.37 | O |
| ATOM | 2101 | N | ASN | L | 53 | 23.065 | −38.780 | 16.673 | 1.00 | 34.32 | N |
| ATOM | 2102 | CA | ASN | L | 53 | 24.123 | −38.327 | 17.561 | 1.00 | 34.78 | C |
| ATOM | 2103 | CB | ASN | L | 53 | 24.789 | −37.070 | 17.032 | 1.00 | 34.27 | C |
| ATOM | 2104 | CG | ASN | L | 53 | 25.314 | −37.237 | 15.620 | 1.00 | 35.67 | C |
| ATOM | 2105 | OD1 | ASN | L | 53 | 24.716 | −36.730 | 14.660 | 1.00 | 38.55 | O |
| ATOM | 2106 | ND2 | ASN | L | 53 | 26.407 | −37.959 | 15.476 | 1.00 | 34.17 | N |
| ATOM | 2107 | C | ASN | L | 53 | 23.562 | −38.076 | 18.954 | 1.00 | 35.46 | C |
| ATOM | 2108 | O | ASN | L | 53 | 22.743 | −37.177 | 19.152 | 1.00 | 35.49 | O |
| ATOM | 2109 | N | LEU | L | 54 | 24.001 | −38.891 | 19.904 | 1.00 | 36.29 | N |
| ATOM | 2110 | CA | LEU | L | 54 | 23.610 | −38.758 | 21.307 | 1.00 | 37.03 | C |
| ATOM | 2111 | CB | LEU | L | 54 | 24.172 | −39.950 | 22.076 | 1.00 | 37.06 | C |
| ATOM | 2112 | CG | LEU | L | 54 | 23.745 | −40.254 | 23.505 | 1.00 | 38.43 | C |
| ATOM | 2113 | CD1 | LEU | L | 54 | 22.239 | −40.480 | 23.555 | 1.00 | 37.60 | C |
| ATOM | 2114 | CD2 | LEU | L | 54 | 24.508 | −41.492 | 24.002 | 1.00 | 36.13 | C |
| ATOM | 2115 | C | LEU | L | 54 | 24.131 | −37.444 | 21.886 | 1.00 | 36.96 | C |
| ATOM | 2116 | O | LEU | L | 54 | 25.335 | −37.220 | 21.923 | 1.00 | 37.63 | O |
| ATOM | 2117 | N | PHE | L | 55 | 23.226 | −36.572 | 22.322 | 1.00 | 37.09 | N |
| ATOM | 2118 | CA | PHE | L | 55 | 23.599 | −35.297 | 22.934 | 1.00 | 37.35 | C |
| ATOM | 2119 | CB | PHE | L | 55 | 22.363 | −34.421 | 23.121 | 1.00 | 36.94 | C |
| ATOM | 2120 | CG | PHE | L | 55 | 22.654 | −33.021 | 23.581 | 1.00 | 37.05 | C |
| ATOM | 2121 | CD1 | PHE | L | 55 | 23.492 | −32.178 | 22.839 | 1.00 | 37.35 | C |
| ATOM | 2122 | CE1 | PHE | L | 55 | 23.753 | −30.861 | 23.257 | 1.00 | 35.75 | C |
| ATOM | 2123 | CZ | PHE | L | 55 | 23.160 | −30.374 | 24.419 | 1.00 | 37.75 | C |
| ATOM | 2124 | CE2 | PHE | L | 55 | 22.295 | −31.202 | 25.163 | 1.00 | 37.26 | C |
| ATOM | 2125 | CD2 | PHE | L | 55 | 22.051 | −32.520 | 24.735 | 1.00 | 37.02 | C |
| ATOM | 2126 | C | PHE | L | 55 | 24.283 | −35.575 | 24.275 | 1.00 | 38.30 | C |
| ATOM | 2127 | O | PHE | L | 55 | 23.926 | −36.522 | 24.970 | 1.00 | 37.44 | O |
| ATOM | 2128 | N | SER | L | 56 | 25.278 | −34.765 | 24.619 | 1.00 | 39.28 | N |
| ATOM | 2129 | CA | SER | L | 56 | 26.070 | −35.010 | 25.823 | 1.00 | 40.87 | C |
| ATOM | 2130 | CB | SER | L | 56 | 27.255 | −34.043 | 25.914 | 1.00 | 41.49 | C |
| ATOM | 2131 | OG | SER | L | 56 | 26.864 | −32.732 | 25.518 | 1.00 | 44.84 | O |
| ATOM | 2132 | C | SER | L | 56 | 25.206 | −34.897 | 27.064 | 1.00 | 40.97 | C |
| ATOM | 2133 | O | SER | L | 56 | 24.429 | −33.952 | 27.215 | 1.00 | 41.07 | O |
| ATOM | 2134 | N | GLY | L | 57 | 25.344 | −35.878 | 27.943 | 1.00 | 41.04 | N |
| ATOM | 2135 | CA | GLY | L | 57 | 24.560 | −35.908 | 29.166 | 1.00 | 41.48 | C |
| ATOM | 2136 | C | GLY | L | 57 | 23.384 | −36.849 | 29.037 | 1.00 | 41.02 | C |
| ATOM | 2137 | O | GLY | L | 57 | 22.777 | −37.219 | 30.036 | 1.00 | 41.27 | O |
| ATOM | 2138 | N | VAL | L | 58 | 23.063 | −37.242 | 27.804 | 1.00 | 40.51 | N |
| ATOM | 2139 | CA | VAL | L | 58 | 21.938 | −38.153 | 27.558 | 1.00 | 39.31 | C |
| ATOM | 2140 | CB | VAL | L | 58 | 21.257 | −37.873 | 26.184 | 1.00 | 39.03 | C |
| ATOM | 2141 | CG1 | VAL | L | 58 | 20.090 | −38.827 | 25.932 | 1.00 | 37.89 | C |
| ATOM | 2142 | CG2 | VAL | L | 58 | 20.787 | −36.438 | 26.118 | 1.00 | 36.80 | C |
| ATOM | 2143 | C | VAL | L | 58 | 22.381 | −39.611 | 27.672 | 1.00 | 39.73 | C |
| ATOM | 2144 | O | VAL | L | 58 | 23.407 | −39.988 | 27.112 | 1.00 | 39.33 | O |
| ATOM | 2145 | N | PRO | L | 59 | 21.591 | −40.436 | 28.389 | 1.00 | 40.28 | N |
| ATOM | 2146 | CA | PRO | L | 59 | 21.917 | −41.844 | 28.595 | 1.00 | 40.81 | C |
| ATOM | 2147 | CB | PRO | L | 59 | 20.793 | −42.343 | 29.513 | 1.00 | 40.67 | C |
| ATOM | 2148 | CG | PRO | L | 59 | 20.282 | −41.145 | 30.180 | 1.00 | 40.71 | C |
| ATOM | 2149 | CD | PRO | L | 59 | 20.410 | −40.032 | 29.175 | 1.00 | 39.99 | C |
| ATOM | 2150 | C | PRO | L | 59 | 21.960 | −42.674 | 27.315 | 1.00 | 41.64 | C |
| ATOM | 2151 | O | PRO | L | 59 | 21.123 | −42.503 | 26.411 | 1.00 | 42.27 | O |
| ATOM | 2152 | N | ASP | L | 60 | 22.951 | −43.560 | 27.282 | 1.00 | 41.90 | N |
| ATOM | 2153 | CA | ASP | L | 60 | 23.258 | −44.492 | 26.213 | 1.00 | 42.83 | C |
| ATOM | 2154 | CB | ASP | L | 60 | 24.300 | −45.480 | 26.745 | 1.00 | 43.97 | C |
| ATOM | 2155 | CG | ASP | L | 60 | 25.039 | −46.209 | 25.641 | 1.00 | 48.16 | C |
| ATOM | 2156 | OD1 | ASP | L | 60 | 25.645 | −45.523 | 24.785 | 1.00 | 53.40 | O |
| ATOM | 2157 | OD2 | ASP | L | 60 | 25.038 | −47.466 | 25.637 | 1.00 | 51.34 | O |
| ATOM | 2158 | C | ASP | L | 60 | 22.070 | −45.297 | 25.698 | 1.00 | 42.24 | C |
| ATOM | 2159 | O | ASP | L | 60 | 22.075 | −45.751 | 24.549 | 1.00 | 42.08 | O |
| ATOM | 2160 | N | ARG | L | 61 | 21.071 | −45.500 | 26.554 | 1.00 | 41.37 | N |
| ATOM | 2161 | CA | ARG | L | 61 | 19.917 | −46.328 | 26.204 | 1.00 | 40.57 | C |
| ATOM | 2162 | CB | ARG | L | 61 | 19.083 | −46.676 | 27.456 | 1.00 | 40.54 | C |
| ATOM | 2163 | CG | ARG | L | 61 | 18.277 | −45.540 | 28.025 | 1.00 | 40.42 | C |
| ATOM | 2164 | CD | ARG | L | 61 | 17.289 | −46.051 | 29.088 | 1.00 | 42.05 | C |
| ATOM | 2165 | NE | ARG | L | 61 | 16.729 | −44.954 | 29.863 | 1.00 | 41.76 | N |
| ATOM | 2166 | CZ | ARG | L | 61 | 17.384 | −44.328 | 30.829 | 1.00 | 43.54 | C |
| ATOM | 2167 | NH1 | ARG | L | 61 | 18.623 | −44.697 | 31.136 | 1.00 | 46.07 | N |
| ATOM | 2168 | NH2 | ARG | L | 61 | 16.807 | −43.333 | 31.491 | 1.00 | 43.85 | N |
| ATOM | 2169 | C | ARG | L | 61 | 19.041 | −45.692 | 25.112 | 1.00 | 39.58 | C |
| ATOM | 2170 | O | ARG | L | 61 | 18.233 | −46.363 | 24.481 | 1.00 | 39.20 | O |
| ATOM | 2171 | N | PHE | L | 62 | 19.203 | −44.395 | 24.912 | 1.00 | 39.15 | N |
| ATOM | 2172 | CA | PHE | L | 62 | 18.550 | −43.716 | 23.810 | 1.00 | 39.12 | C |
| ATOM | 2173 | CB | PHE | L | 62 | 18.333 | −42.254 | 24.155 | 1.00 | 38.71 | C |
| ATOM | 2174 | CG | PHE | L | 62 | 17.358 | −42.038 | 25.275 | 1.00 | 38.80 | C |
| ATOM | 2175 | CD1 | PHE | L | 62 | 15.984 | −42.021 | 25.024 | 1.00 | 37.91 | C |
| ATOM | 2176 | CE1 | PHE | L | 62 | 15.068 | −41.818 | 26.053 | 1.00 | 36.99 | C |
| ATOM | 2177 | CZ | PHE | L | 62 | 15.521 | −41.651 | 27.372 | 1.00 | 38.64 | C |
| ATOM | 2178 | CE2 | PHE | L | 62 | 16.894 | −41.688 | 27.644 | 1.00 | 39.07 | C |
| ATOM | 2179 | CD2 | PHE | L | 62 | 17.807 | −41.880 | 26.590 | 1.00 | 38.57 | C |
| ATOM | 2180 | C | PHE | L | 62 | 19.420 | −43.831 | 22.565 | 1.00 | 39.32 | C |
| ATOM | 2181 | O | PHE | L | 62 | 20.645 | −43.637 | 22.634 | 1.00 | 39.31 | O |
| ATOM | 2182 | N | SER | L | 63 | 18.796 | −44.171 | 21.439 | 1.00 | 38.91 | N |
| ATOM | 2183 | CA | SER | L | 63 | 19.482 | −44.139 | 20.139 | 1.00 | 38.42 | C |
| ATOM | 2184 | CB | SER | L | 63 | 20.240 | −45.434 | 19.874 | 1.00 | 38.29 | C |
| ATOM | 2185 | OG | SER | L | 63 | 19.356 | −46.526 | 19.738 | 1.00 | 38.30 | O |
| ATOM | 2186 | C | SER | L | 63 | 18.538 | −43.816 | 18.969 | 1.00 | 38.26 | C |
| ATOM | 2187 | O | SER | L | 63 | 17.368 | −44.203 | 18.982 | 1.00 | 38.25 | O |
| ATOM | 2188 | N | GLY | L | 64 | 19.071 | −43.111 | 17.968 | 1.00 | 37.47 | N |
| ATOM | 2189 | CA | GLY | L | 64 | 18.328 | −42.783 | 16.747 | 1.00 | 36.49 | C |
| ATOM | 2190 | C | GLY | L | 64 | 18.923 | −43.370 | 15.472 | 1.00 | 35.86 | C |
| ATOM | 2191 | O | GLY | L | 64 | 20.146 | −43.557 | 15.354 | 1.00 | 34.36 | O |
| ATOM | 2192 | N | SER | L | 65 | 18.037 | −43.693 | 14.533 | 1.00 | 35.14 | N |
| ATOM | 2193 | CA | SER | L | 65 | 18.431 | −44.046 | 13.169 | 1.00 | 35.22 | C |
| ATOM | 2194 | CB | SER | L | 65 | 18.502 | −45.551 | 12.999 | 1.00 | 34.55 | C |
| ATOM | 2195 | OG | SER | L | 65 | 17.212 | −46.108 | 13.125 | 1.00 | 35.93 | O |
| ATOM | 2196 | C | SER | L | 65 | 17.463 | −43.427 | 12.138 | 1.00 | 35.46 | C |
| ATOM | 2197 | O | SER | L | 65 | 16.563 | −42.650 | 12.497 | 1.00 | 35.23 | O |
| ATOM | 2198 | N | GLY | L | 66 | 17.660 | −43.773 | 10.868 | 1.00 | 35.74 | N |
| ATOM | 2199 | CA | GLY | L | 66 | 16.805 | −43.308 | 9.782 | 1.00 | 36.24 | C |
| ATOM | 2200 | C | GLY | L | 66 | 17.556 | −42.550 | 8.704 | 1.00 | 36.64 | C |
| ATOM | 2201 | O | GLY | L | 66 | 18.770 | −42.327 | 8.812 | 1.00 | 36.56 | O |
| ATOM | 2202 | N | SER | L | 67 | 16.823 | −42.148 | 7.660 | 1.00 | 36.59 | N |
| ATOM | 2203 | CA | SER | L | 67 | 17.366 | −41.330 | 6.567 | 1.00 | 36.57 | C |
| ATOM | 2204 | CB | SER | L | 67 | 18.352 | −42.139 | 5.705 | 1.00 | 36.79 | C |
| ATOM | 2205 | OG | SER | L | 67 | 17.665 | −43.143 | 4.979 | 1.00 | 37.31 | O |
| ATOM | 2206 | C | SER | L | 67 | 16.237 | −40.810 | 5.692 | 1.00 | 36.31 | C |
| ATOM | 2207 | O | SER | L | 67 | 15.166 | −41.415 | 5.633 | 1.00 | 36.31 | O |
| ATOM | 2208 | N | GLY | L | 68 | 16.481 | −39.693 | 5.013 | 1.00 | 35.88 | N |
| ATOM | 2209 | CA | GLY | L | 68 | 15.486 | −39.124 | 4.095 | 1.00 | 35.27 | C |
| ATOM | 2210 | C | GLY | L | 68 | 14.299 | −38.515 | 4.807 | 1.00 | 34.40 | C |
| ATOM | 2211 | O | GLY | L | 68 | 14.349 | −37.364 | 5.260 | 1.00 | 34.21 | O |
| ATOM | 2212 | N | THR | L | 69 | 13.237 | −39.307 | 4.896 | 1.00 | 33.90 | N |
| ATOM | 2213 | CA | THR | L | 69 | 11.971 | −38.913 | 5.496 | 1.00 | 33.68 | C |
| ATOM | 2214 | CB | THR | L | 69 | 10.832 | −38.914 | 4.440 | 1.00 | 33.42 | C |
| ATOM | 2215 | OG1 | THR | L | 69 | 10.705 | −40.233 | 3.908 | 1.00 | 31.51 | O |
| ATOM | 2216 | CG2 | THR | L | 69 | 11.127 | −37.970 | 3.292 | 1.00 | 34.75 | C |
| ATOM | 2217 | C | THR | L | 69 | 11.535 | −39.866 | 6.630 | 1.00 | 33.14 | C |
| ATOM | 2218 | O | THR | L | 69 | 10.574 | −39.578 | 7.317 | 1.00 | 33.24 | O |
| ATOM | 2219 | N | ASP | L | 70 | 12.198 | −41.008 | 6.787 | 1.00 | 33.30 | N |
| ATOM | 2220 | CA | ASP | L | 70 | 11.806 | −41.982 | 7.819 | 1.00 | 34.55 | C |
| ATOM | 2221 | CB | ASP | L | 70 | 11.718 | −43.402 | 7.258 | 1.00 | 35.01 | C |
| ATOM | 2222 | CG | ASP | L | 70 | 10.877 | −43.505 | 6.006 | 1.00 | 38.22 | C |
| ATOM | 2223 | OD1 | ASP | L | 70 | 9.795 | −42.863 | 5.910 | 1.00 | 40.07 | O |
| ATOM | 2224 | OD2 | ASP | L | 70 | 11.318 | −44.255 | 5.112 | 1.00 | 40.67 | O |
| ATOM | 2225 | C | ASP | L | 70 | 12.829 | −42.018 | 8.958 | 1.00 | 33.90 | C |
| ATOM | 2226 | O | ASP | L | 70 | 14.003 | −42.254 | 8.720 | 1.00 | 33.74 | O |
| ATOM | 2227 | N | PHE | L | 71 | 12.368 | −41.826 | 10.187 | 1.00 | 33.64 | N |
| ATOM | 2228 | CA | PHE | L | 71 | 13.252 | −41.795 | 11.364 | 1.00 | 33.32 | C |
| ATOM | 2229 | CB | PHE | L | 71 | 13.521 | −40.338 | 11.744 | 1.00 | 32.77 | C |
| ATOM | 2230 | CG | PHE | L | 71 | 14.055 | −39.522 | 10.600 | 1.00 | 33.28 | C |
| ATOM | 2231 | CD1 | PHE | L | 71 | 13.189 | −38.870 | 9.723 | 1.00 | 33.12 | C |
| ATOM | 2232 | CE1 | PHE | L | 71 | 13.685 | −38.144 | 8.634 | 1.00 | 34.27 | C |
| ATOM | 2233 | CZ | PHE | L | 71 | 15.071 | −38.064 | 8.428 | 1.00 | 35.92 | C |
| ATOM | 2234 | CE2 | PHE | L | 71 | 15.932 | −38.722 | 9.307 | 1.00 | 34.96 | C |
| ATOM | 2235 | CD2 | PHE | L | 71 | 15.416 | −39.452 | 10.374 | 1.00 | 32.32 | C |
| ATOM | 2236 | C | PHE | L | 71 | 12.667 | −42.574 | 12.549 | 1.00 | 33.33 | C |
| ATOM | 2237 | O | PHE | L | 71 | 11.450 | −42.573 | 12.742 | 1.00 | 32.79 | O |
| ATOM | 2238 | N | THR | L | 72 | 13.543 | −43.209 | 13.338 | 1.00 | 33.38 | N |
| ATOM | 2239 | CA | THR | L | 72 | 13.152 | −44.037 | 14.493 | 1.00 | 33.71 | C |
| ATOM | 2240 | CB | THR | L | 72 | 13.291 | −45.547 | 14.181 | 1.00 | 33.72 | C |
| ATOM | 2241 | OG1 | THR | L | 72 | 12.575 | −45.854 | 12.980 | 1.00 | 35.36 | O |
| ATOM | 2242 | CG2 | THR | L | 72 | 12.739 | −46.424 | 15.320 | 1.00 | 33.59 | C |
| ATOM | 2243 | C | THR | L | 72 | 14.000 | −43.675 | 15.727 | 1.00 | 33.77 | C |
| ATOM | 2244 | O | THR | L | 72 | 15.237 | −43.638 | 15.637 | 1.00 | 33.57 | O |
| ATOM | 2245 | N | LEU | L | 73 | 13.326 | −43.350 | 16.838 | 1.00 | 33.63 | N |
| ATOM | 2246 | CA | LEU | L | 73 | 13.955 | −43.275 | 18.171 | 1.00 | 34.24 | C |
| ATOM | 2247 | CB | LEU | L | 73 | 13.387 | −42.115 | 18.992 | 1.00 | 33.89 | C |
| ATOM | 2248 | CG | LEU | L | 73 | 13.932 | −41.793 | 20.395 | 1.00 | 32.65 | C |
| ATOM | 2249 | CD1 | LEU | L | 73 | 15.344 | −41.173 | 20.345 | 1.00 | 27.95 | C |
| ATOM | 2250 | CD2 | LEU | L | 73 | 12.960 | −40.847 | 21.116 | 1.00 | 31.05 | C |
| ATOM | 2251 | C | LEU | L | 73 | 13.720 | −44.599 | 18.902 | 1.00 | 35.33 | C |
| ATOM | 2252 | O | LEU | L | 73 | 12.599 | −45.105 | 18.941 | 1.00 | 35.16 | O |
| ATOM | 2253 | N | LYS | L | 74 | 14.786 | −45.175 | 19.441 | 1.00 | 36.72 | N |
| ATOM | 2254 | CA | LYS | L | 74 | 14.675 | −46.378 | 20.248 | 1.00 | 38.33 | C |
| ATOM | 2255 | CB | LYS | L | 74 | 15.423 | −47.560 | 19.617 | 1.00 | 38.67 | C |
| ATOM | 2256 | CG | LYS | L | 74 | 14.766 | −48.101 | 18.349 | 1.00 | 41.24 | C |
| ATOM | 2257 | CD | LYS | L | 74 | 15.486 | −49.347 | 17.835 | 1.00 | 44.50 | C |
| ATOM | 2258 | CE | LYS | L | 74 | 14.982 | −49.728 | 16.447 | 1.00 | 47.26 | C |
| ATOM | 2259 | NZ | LYS | L | 74 | 15.582 | −51.016 | 15.978 | 1.00 | 48.52 | N |
| ATOM | 2260 | C | LYS | L | 74 | 15.192 | −46.134 | 21.662 | 1.00 | 38.88 | C |
| ATOM | 2261 | O | LYS | L | 74 | 16.200 | −45.436 | 21.871 | 1.00 | 38.55 | O |
| ATOM | 2262 | N | ILE | L | 75 | 14.460 | −46.699 | 22.619 | 1.00 | 39.38 | N |
| ATOM | 2263 | CA | ILE | L | 75 | 14.852 | −46.704 | 24.020 | 1.00 | 39.52 | C |
| ATOM | 2264 | CB | ILE | L | 75 | 13.794 | −46.084 | 24.948 | 1.00 | 39.63 | C |
| ATOM | 2265 | CG1 | ILE | L | 75 | 13.326 | −44.732 | 24.391 | 1.00 | 39.40 | C |
| ATOM | 2266 | CD1 | ILE | L | 75 | 12.095 | −44.125 | 25.070 | 1.00 | 38.26 | C |
| ATOM | 2267 | CG2 | ILE | L | 75 | 14.393 | −45.891 | 26.360 | 1.00 | 40.11 | C |
| ATOM | 2268 | C | ILE | L | 75 | 15.104 | −48.148 | 24.381 | 1.00 | 40.02 | C |
| ATOM | 2269 | O | ILE | L | 75 | 14.195 | −49.010 | 24.306 | 1.00 | 39.74 | O |
| ATOM | 2270 | N | SER | L | 76 | 16.359 | −48.400 | 24.736 | 1.00 | 40.02 | N |
| ATOM | 2271 | CA | SER | L | 76 | 16.866 | −49.741 | 25.011 | 1.00 | 41.27 | C |
| ATOM | 2272 | CB | SER | L | 76 | 18.342 | −49.636 | 25.412 | 1.00 | 41.28 | C |
| ATOM | 2273 | OG | SER | L | 76 | 18.837 | −50.909 | 25.735 | 1.00 | 43.91 | O |
| ATOM | 2274 | C | SER | L | 76 | 16.088 | −50.524 | 26.085 | 1.00 | 40.80 | C |
| ATOM | 2275 | O | SER | L | 76 | 15.733 | −51.688 | 25.879 | 1.00 | 41.20 | O |
| ATOM | 2276 | N | ARG | L | 77 | 15.853 | −49.884 | 27.228 | 1.00 | 40.58 | N |
| ATOM | 2277 | CA | ARG | L | 77 | 15.173 | −50.502 | 28.371 | 1.00 | 40.90 | C |
| ATOM | 2278 | CB | ARG | L | 77 | 16.179 | −51.218 | 29.293 | 1.00 | 41.17 | C |
| ATOM | 2279 | CG | ARG | L | 77 | 16.735 | −52.550 | 28.756 | 1.00 | 44.11 | C |
| ATOM | 2280 | CD | ARG | L | 77 | 17.905 | −53.152 | 29.631 | 1.00 | 49.94 | C |
| ATOM | 2281 | NE | ARG | L | 77 | 18.859 | −52.137 | 30.124 | 1.00 | 53.02 | N |
| ATOM | 2282 | CZ | ARG | L | 77 | 19.825 | −51.562 | 29.397 | 1.00 | 55.69 | C |
| ATOM | 2283 | NH1 | ARG | L | 77 | 20.001 | −51.885 | 28.117 | 1.00 | 55.41 | N |
| ATOM | 2284 | NH2 | ARG | L | 77 | 20.619 | −50.647 | 29.950 | 1.00 | 55.79 | N |
| ATOM | 2285 | C | ARG | L | 77 | 14.511 | −49.361 | 29.113 | 1.00 | 40.12 | C |
| ATOM | 2286 | O | ARG | L | 77 | 15.194 | −48.572 | 29.765 | 1.00 | 41.16 | O |
| ATOM | 2287 | N | VAL | L | 78 | 13.195 | −49.244 | 28.997 | 1.00 | 39.25 | N |
| ATOM | 2288 | CA | VAL | L | 78 | 12.508 | −48.084 | 29.542 | 1.00 | 38.93 | C |
| ATOM | 2289 | CB | VAL | L | 78 | 11.066 | −47.951 | 28.984 | 1.00 | 39.29 | C |
| ATOM | 2290 | CG1 | VAL | L | 78 | 10.200 | −47.053 | 29.877 | 1.00 | 38.22 | C |
| ATOM | 2291 | CG2 | VAL | L | 78 | 11.103 | −47.385 | 27.537 | 1.00 | 37.84 | C |
| ATOM | 2292 | C | VAL | L | 78 | 12.497 | −48.086 | 31.069 | 1.00 | 39.76 | C |
| ATOM | 2293 | O | VAL | L | 78 | 12.339 | −49.134 | 31.691 | 1.00 | 38.51 | O |
| ATOM | 2294 | N | GLU | L | 79 | 12.637 | −46.882 | 31.630 | 1.00 | 40.55 | N |
| ATOM | 2295 | CA | GLU | L | 79 | 12.655 | −46.625 | 33.071 | 1.00 | 41.79 | C |
| ATOM | 2296 | CB | GLU | L | 79 | 14.085 | −46.378 | 33.554 | 1.00 | 41.89 | C |
| ATOM | 2297 | CG | GLU | L | 79 | 15.034 | −47.538 | 33.302 | 1.00 | 45.45 | C |
| ATOM | 2298 | CD | GLU | L | 79 | 16.443 | −47.254 | 33.786 | 1.00 | 48.97 | C |
| ATOM | 2299 | OE1 | GLU | L | 79 | 16.677 | −46.151 | 34.325 | 1.00 | 49.93 | O |
| ATOM | 2300 | OE2 | GLU | L | 79 | 17.316 | −48.132 | 33.627 | 1.00 | 49.89 | O |
| ATOM | 2301 | C | GLU | L | 79 | 11.759 | −45.428 | 33.430 | 1.00 | 41.94 | C |
| ATOM | 2302 | O | GLU | L | 79 | 11.455 | −44.594 | 32.578 | 1.00 | 41.71 | O |
| ATOM | 2303 | N | ALA | L | 80 | 11.336 | −45.362 | 34.691 | 1.00 | 42.03 | N |
| ATOM | 2304 | CA | ALA | L | 80 | 10.409 | −44.345 | 35.174 | 1.00 | 42.08 | C |
| ATOM | 2305 | CB | ALA | L | 80 | 10.285 | −44.427 | 36.715 | 1.00 | 42.44 | C |
| ATOM | 2306 | C | ALA | L | 80 | 10.794 | −42.939 | 34.770 | 1.00 | 41.97 | C |
| ATOM | 2307 | O | ALA | L | 80 | 9.934 | −42.104 | 34.523 | 1.00 | 42.61 | O |
| ATOM | 2308 | N | GLU | L | 81 | 12.090 | −42.668 | 34.716 | 1.00 | 42.02 | N |
| ATOM | 2309 | CA | GLU | L | 81 | 12.558 | −41.350 | 34.313 | 1.00 | 42.28 | C |
| ATOM | 2310 | CB | GLU | L | 81 | 14.024 | −41.137 | 34.731 | 1.00 | 42.69 | C |
| ATOM | 2311 | CG | GLU | L | 81 | 15.027 | −42.217 | 34.253 | 1.00 | 45.77 | C |
| ATOM | 2312 | CD | GLU | L | 81 | 15.109 | −43.462 | 35.166 | 1.00 | 50.40 | C |
| ATOM | 2313 | OE1 | GLU | L | 81 | 16.033 | −44.292 | 34.945 | 1.00 | 50.09 | O |
| ATOM | 2314 | OE2 | GLU | L | 81 | 14.259 | −43.620 | 36.087 | 1.00 | 50.34 | O |
| ATOM | 2315 | C | GLU | L | 81 | 12.335 | −41.075 | 32.799 | 1.00 | 41.42 | C |
| ATOM | 2316 | O | GLU | L | 81 | 12.467 | −39.934 | 32.355 | 1.00 | 41.63 | O |
| ATOM | 2317 | N | ASP | L | 82 | 11.964 | −42.105 | 32.035 | 1.00 | 39.96 | N |
| ATOM | 2318 | CA | ASP | L | 82 | 11.751 | −41.954 | 30.582 | 1.00 | 39.04 | C |
| ATOM | 2319 | CB | ASP | L | 82 | 12.091 | −43.246 | 29.835 | 1.00 | 38.56 | C |
| ATOM | 2320 | CG | ASP | L | 82 | 13.513 | −43.699 | 30.078 | 1.00 | 37.68 | C |
| ATOM | 2321 | OD1 | ASP | L | 82 | 14.337 | −42.849 | 30.462 | 1.00 | 37.45 | O |
| ATOM | 2322 | OD2 | ASP | L | 82 | 13.812 | −44.899 | 29.878 | 1.00 | 37.40 | O |
| ATOM | 2323 | C | ASP | L | 82 | 10.352 | −41.476 | 30.193 | 1.00 | 38.65 | C |
| ATOM | 2324 | O | ASP | L | 82 | 10.110 | −41.192 | 29.022 | 1.00 | 38.71 | O |
| ATOM | 2325 | N | VAL | L | 83 | 9.453 | −41.381 | 31.175 | 1.00 | 37.72 | N |
| ATOM | 2326 | CA | VAL | L | 83 | 8.057 | −40.987 | 30.962 | 1.00 | 36.70 | C |
| ATOM | 2327 | CB | VAL | L | 83 | 7.210 | −41.167 | 32.254 | 1.00 | 37.23 | C |
| ATOM | 2328 | CG1 | VAL | L | 83 | 5.804 | −40.640 | 32.069 | 1.00 | 37.00 | C |
| ATOM | 2329 | CG2 | VAL | L | 83 | 7.142 | −42.623 | 32.653 | 1.00 | 36.81 | C |
| ATOM | 2330 | C | VAL | L | 83 | 7.931 | −39.546 | 30.483 | 1.00 | 36.26 | C |
| ATOM | 2331 | O | VAL | L | 83 | 8.677 | −38.676 | 30.934 | 1.00 | 36.90 | O |
| ATOM | 2332 | N | GLY | L | 84 | 6.974 | −39.302 | 29.580 | 1.00 | 34.86 | N |
| ATOM | 2333 | CA | GLY | L | 84 | 6.704 | −37.962 | 29.045 | 1.00 | 33.56 | C |
| ATOM | 2334 | C | GLY | L | 84 | 6.421 | −37.942 | 27.548 | 1.00 | 33.02 | C |
| ATOM | 2335 | O | GLY | L | 84 | 6.196 | −38.982 | 26.933 | 1.00 | 32.80 | O |
| ATOM | 2336 | N | VAL | L | 85 | 6.421 | −36.759 | 26.953 | 1.00 | 32.34 | N |
| ATOM | 2337 | CA | VAL | L | 85 | 6.115 | −36.647 | 25.539 | 1.00 | 31.93 | C |
| ATOM | 2338 | CB | VAL | L | 85 | 5.092 | −35.516 | 25.232 | 1.00 | 31.76 | C |
| ATOM | 2339 | CG1 | VAL | L | 85 | 4.767 | −35.457 | 23.734 | 1.00 | 31.04 | C |
| ATOM | 2340 | CG2 | VAL | L | 85 | 3.818 | −35.732 | 26.003 | 1.00 | 31.59 | C |
| ATOM | 2341 | C | VAL | L | 85 | 7.412 | −36.492 | 24.724 | 1.00 | 32.16 | C |
| ATOM | 2342 | O | VAL | L | 85 | 8.189 | −35.550 | 24.943 | 1.00 | 32.11 | O |
| ATOM | 2343 | N | TYR | L | 86 | 7.628 | −37.441 | 23.817 | 1.00 | 30.82 | N |
| ATOM | 2344 | CA | TYR | L | 86 | 8.729 | −37.430 | 22.882 | 1.00 | 30.82 | C |
| ATOM | 2345 | CB | TYR | L | 86 | 9.179 | −38.862 | 22.597 | 1.00 | 30.41 | C |
| ATOM | 2346 | CG | TYR | L | 86 | 9.832 | −39.549 | 23.778 | 1.00 | 31.70 | C |
| ATOM | 2347 | CD1 | TYR | L | 86 | 9.060 | −40.230 | 24.735 | 1.00 | 32.92 | C |
| ATOM | 2348 | CE1 | TYR | L | 86 | 9.660 | −40.871 | 25.805 | 1.00 | 32.25 | C |
| ATOM | 2349 | CZ | TYR | L | 86 | 11.040 | −40.831 | 25.940 | 1.00 | 32.57 | C |
| ATOM | 2350 | OH | TYR | L | 86 | 11.628 | −41.452 | 27.010 | 1.00 | 34.42 | O |
| ATOM | 2351 | CE2 | TYR | L | 86 | 11.826 | −40.155 | 25.016 | 1.00 | 31.40 | C |
| ATOM | 2352 | CD2 | TYR | L | 86 | 11.216 | −39.524 | 23.940 | 1.00 | 31.29 | C |
| ATOM | 2353 | C | TYR | L | 86 | 8.315 | −36.745 | 21.582 | 1.00 | 30.79 | C |
| ATOM | 2354 | O | TYR | L | 86 | 7.286 | −37.101 | 20.980 | 1.00 | 31.87 | O |
| ATOM | 2355 | N | PHE | L | 87 | 9.099 | −35.754 | 21.166 | 1.00 | 30.38 | N |
| ATOM | 2356 | CA | PHE | L | 87 | 8.867 | −35.001 | 19.921 | 1.00 | 29.75 | C |
| ATOM | 2357 | CB | PHE | L | 87 | 8.772 | −33.488 | 20.185 | 1.00 | 29.26 | C |
| ATOM | 2358 | CG | PHE | L | 87 | 7.553 | −33.053 | 20.953 | 1.00 | 29.72 | C |
| ATOM | 2359 | CD1 | PHE | L | 87 | 6.336 | −32.846 | 20.313 | 1.00 | 28.86 | C |
| ATOM | 2360 | CE1 | PHE | L | 87 | 5.210 | −32.435 | 21.031 | 1.00 | 29.97 | C |
| ATOM | 2361 | CZ | PHE | L | 87 | 5.301 | −32.205 | 22.389 | 1.00 | 30.21 | C |
| ATOM | 2362 | CE2 | PHE | L | 87 | 6.517 | −32.400 | 23.036 | 1.00 | 29.65 | C |
| ATOM | 2363 | CD2 | PHE | L | 87 | 7.631 | −32.815 | 22.317 | 1.00 | 28.73 | C |
| ATOM | 2364 | C | PHE | L | 87 | 10.039 | −35.168 | 18.950 | 1.00 | 29.92 | C |
| ATOM | 2365 | O | PHE | L | 87 | 11.201 | −35.010 | 19.353 | 1.00 | 28.60 | O |
| ATOM | 2366 | N | CYS | L | 88 | 9.751 | −35.441 | 17.668 | 1.00 | 28.92 | N |
| ATOM | 2367 | CA | CYS | L | 88 | 10.772 | −35.206 | 16.656 | 1.00 | 28.43 | C |
| ATOM | 2368 | CB | CYS | L | 88 | 10.644 | −36.156 | 15.455 | 1.00 | 29.09 | C |
| ATOM | 2369 | SG | CYS | L | 88 | 9.014 | −36.171 | 14.601 | 1.00 | 30.17 | S |
| ATOM | 2370 | C | CYS | L | 88 | 10.727 | −33.732 | 16.236 | 1.00 | 27.81 | C |
| ATOM | 2371 | O | CYS | L | 88 | 9.708 | −33.063 | 16.384 | 1.00 | 27.95 | O |
| ATOM | 2372 | N | SER | L | 89 | 11.843 | −33.237 | 15.710 | 1.00 | 27.40 | N |
| ATOM | 2373 | CA | SER | L | 89 | 12.029 | −31.819 | 15.388 | 1.00 | 26.70 | C |
| ATOM | 2374 | CB | SER | L | 89 | 12.815 | −31.152 | 16.530 | 1.00 | 26.78 | C |
| ATOM | 2375 | OG | SER | L | 89 | 13.255 | −29.846 | 16.185 | 1.00 | 25.65 | O |
| ATOM | 2376 | C | SER | L | 89 | 12.883 | −31.710 | 14.134 | 1.00 | 26.82 | C |
| ATOM | 2377 | O | SER | L | 89 | 13.984 | −32.256 | 14.106 | 1.00 | 27.57 | O |
| ATOM | 2378 | N | GLN | L | 90 | 12.411 | −30.994 | 13.119 | 1.00 | 26.67 | N |
| ATOM | 2379 | CA | GLN | L | 90 | 13.228 | −30.795 | 11.916 | 1.00 | 27.06 | C |
| ATOM | 2380 | CB | GLN | L | 90 | 12.476 | −31.213 | 10.614 | 1.00 | 26.96 | C |
| ATOM | 2381 | CG | GLN | L | 90 | 11.351 | −30.271 | 10.135 | 1.00 | 26.18 | C |
| ATOM | 2382 | CD | GLN | L | 90 | 11.857 | −29.043 | 9.361 | 1.00 | 26.03 | C |
| ATOM | 2383 | OE1 | GLN | L | 90 | 12.955 | −29.053 | 8.834 | 1.00 | 27.34 | O |
| ATOM | 2384 | NE2 | GLN | L | 90 | 11.061 | −27.996 | 9.307 | 1.00 | 24.81 | N |
| ATOM | 2385 | C | GLN | L | 90 | 13.695 | −29.355 | 11.876 | 1.00 | 26.80 | C |
| ATOM | 2386 | O | GLN | L | 90 | 12.922 | −28.441 | 12.151 | 1.00 | 26.43 | O |
| ATOM | 2387 | N | SER | L | 91 | 14.966 | −29.147 | 11.568 | 1.00 | 27.03 | N |
| ATOM | 2388 | CA | SER | L | 91 | 15.436 | −27.790 | 11.307 | 1.00 | 27.44 | C |
| ATOM | 2389 | CB | SER | L | 91 | 16.328 | −27.267 | 12.452 | 1.00 | 27.04 | C |
| ATOM | 2390 | OG | SER | L | 91 | 17.456 | −28.104 | 12.615 | 1.00 | 27.35 | O |
| ATOM | 2391 | C | SER | L | 91 | 16.127 | −27.636 | 9.952 | 1.00 | 28.24 | C |
| ATOM | 2392 | O | SER | L | 91 | 16.995 | −26.772 | 9.772 | 1.00 | 28.88 | O |
| ATOM | 2393 | N | THR | L | 92 | 15.714 | −28.439 | 8.983 | 1.00 | 29.19 | N |
| ATOM | 2394 | CA | THR | L | 92 | 16.241 | −28.312 | 7.630 | 1.00 | 29.75 | C |
| ATOM | 2395 | CB | THR | L | 92 | 16.005 | −29.601 | 6.851 | 1.00 | 29.46 | C |
| ATOM | 2396 | OG1 | THR | L | 92 | 16.693 | −30.662 | 7.518 | 1.00 | 29.55 | O |
| ATOM | 2397 | CG2 | THR | L | 92 | 16.531 | −29.486 | 5.428 | 1.00 | 28.61 | C |
| ATOM | 2398 | C | THR | L | 92 | 15.571 | −27.143 | 6.914 | 1.00 | 30.73 | C |
| ATOM | 2399 | O | THR | L | 92 | 16.236 | −26.331 | 6.242 | 1.00 | 30.76 | O |
| ATOM | 2400 | N | HIS | L | 93 | 14.252 | −27.065 | 7.066 | 1.00 | 31.02 | N |
| ATOM | 2401 | CA | HIS | L | 93 | 13.449 | −26.114 | 6.315 | 1.00 | 31.87 | C |
| ATOM | 2402 | CB | HIS | L | 93 | 12.373 | −26.853 | 5.506 | 1.00 | 32.44 | C |
| ATOM | 2403 | CG | HIS | L | 93 | 12.907 | −27.941 | 4.623 | 1.00 | 31.67 | C |
| ATOM | 2404 | ND1 | HIS | L | 93 | 13.493 | −27.688 | 3.404 | 1.00 | 35.63 | N |
| ATOM | 2405 | CE1 | HIS | L | 93 | 13.862 | −28.828 | 2.843 | 1.00 | 33.92 | C |
| ATOM | 2406 | NE2 | HIS | L | 93 | 13.530 | −29.813 | 3.656 | 1.00 | 36.14 | N |
| ATOM | 2407 | CD2 | HIS | L | 93 | 12.927 | −29.283 | 4.775 | 1.00 | 34.21 | C |
| ATOM | 2408 | C | HIS | L | 93 | 12.757 | −25.171 | 7.240 | 1.00 | 31.92 | C |
| ATOM | 2409 | O | HIS | L | 93 | 12.221 | −25.593 | 8.264 | 1.00 | 31.77 | O |
| ATOM | 2410 | N | PHE | L | 94 | 12.758 | −23.900 | 6.856 | 1.00 | 31.77 | N |
| ATOM | 2411 | CA | PHE | L | 94 | 11.901 | −22.893 | 7.461 | 1.00 | 32.33 | C |
| ATOM | 2412 | CB | PHE | L | 94 | 12.448 | −21.495 | 7.169 | 1.00 | 32.77 | C |
| ATOM | 2413 | CG | PHE | L | 94 | 13.689 | −21.126 | 7.955 | 1.00 | 34.73 | C |
| ATOM | 2414 | CD1 | PHE | L | 94 | 13.684 | −21.120 | 9.350 | 1.00 | 35.77 | C |
| ATOM | 2415 | CE1 | PHE | L | 94 | 14.816 | −20.734 | 10.077 | 1.00 | 36.90 | C |
| ATOM | 2416 | CZ | PHE | L | 94 | 15.971 | −20.340 | 9.413 | 1.00 | 38.16 | C |
| ATOM | 2417 | CE2 | PHE | L | 94 | 15.987 | −20.315 | 8.011 | 1.00 | 37.68 | C |
| ATOM | 2418 | CD2 | PHE | L | 94 | 14.841 | −20.712 | 7.292 | 1.00 | 37.79 | C |
| ATOM | 2419 | C | PHE | L | 94 | 10.462 | −22.984 | 6.911 | 1.00 | 31.58 | C |
| ATOM | 2420 | O | PHE | L | 94 | 10.272 | −23.131 | 5.699 | 1.00 | 32.12 | O |
| ATOM | 2421 | N | PRO | L | 95 | 9.444 | −22.873 | 7.780 | 1.00 | 30.54 | N |
| ATOM | 2422 | CA | PRO | L | 95 | 9.538 | −22.731 | 9.232 | 1.00 | 29.65 | C |
| ATOM | 2423 | CB | PRO | L | 95 | 8.133 | −22.258 | 9.632 | 1.00 | 29.26 | C |
| ATOM | 2424 | CG | PRO | L | 95 | 7.224 | −22.823 | 8.577 | 1.00 | 30.80 | C |
| ATOM | 2425 | CD | PRO | L | 95 | 8.051 | −22.740 | 7.297 | 1.00 | 30.57 | C |
| ATOM | 2426 | C | PRO | L | 95 | 9.900 | −24.058 | 9.916 | 1.00 | 28.71 | C |
| ATOM | 2427 | O | PRO | L | 95 | 9.529 | −25.125 | 9.435 | 1.00 | 28.44 | O |
| ATOM | 2428 | N | PHE | L | 96 | 10.660 | −24.002 | 11.012 | 1.00 | 27.97 | N |
| ATOM | 2429 | CA | PHE | L | 96 | 11.002 | −25.241 | 11.732 | 1.00 | 27.75 | C |
| ATOM | 2430 | CB | PHE | L | 96 | 11.986 | −24.990 | 12.884 | 1.00 | 28.07 | C |
| ATOM | 2431 | CG | PHE | L | 96 | 13.356 | −24.538 | 12.418 | 1.00 | 31.11 | C |
| ATOM | 2432 | CD1 | PHE | L | 96 | 14.312 | −24.080 | 13.334 | 1.00 | 32.55 | C |
| ATOM | 2433 | CE1 | PHE | L | 96 | 15.576 | −23.655 | 12.902 | 1.00 | 31.79 | C |
| ATOM | 2434 | CZ | PHE | L | 96 | 15.896 | −23.691 | 11.522 | 1.00 | 33.90 | C |
| ATOM | 2435 | CE2 | PHE | L | 96 | 14.936 | −24.131 | 10.589 | 1.00 | 34.03 | C |
| ATOM | 2436 | CD2 | PHE | L | 96 | 13.683 | −24.553 | 11.042 | 1.00 | 33.41 | C |
| ATOM | 2437 | C | PHE | L | 96 | 9.717 | −25.889 | 12.218 | 1.00 | 26.51 | C |
| ATOM | 2438 | O | PHE | L | 96 | 8.757 | −25.197 | 12.513 | 1.00 | 25.77 | O |
| ATOM | 2439 | N | THR | L | 97 | 9.671 | −27.213 | 12.242 | 1.00 | 26.11 | N |
| ATOM | 2440 | CA | THR | L | 97 | 8.426 | −27.871 | 12.620 | 1.00 | 26.31 | C |
| ATOM | 2441 | CB | THR | L | 97 | 7.632 | −28.390 | 11.381 | 1.00 | 25.92 | C |
| ATOM | 2442 | OG1 | THR | L | 97 | 8.545 | −29.035 | 10.496 | 1.00 | 24.18 | O |
| ATOM | 2443 | CG2 | THR | L | 97 | 6.921 | −27.229 | 10.649 | 1.00 | 25.75 | C |
| ATOM | 2444 | C | THR | L | 97 | 8.697 | −29.044 | 13.520 | 1.00 | 26.11 | C |
| ATOM | 2445 | O | THR | L | 97 | 9.813 | −29.544 | 13.573 | 1.00 | 26.31 | O |
| ATOM | 2446 | N | PHE | L | 98 | 7.639 | −29.480 | 14.193 | 1.00 | 26.25 | N |
| ATOM | 2447 | CA | PHE | L | 98 | 7.680 | −30.548 | 15.181 | 1.00 | 26.45 | C |
| ATOM | 2448 | CB | PHE | L | 98 | 7.441 | −29.938 | 16.566 | 1.00 | 26.36 | C |
| ATOM | 2449 | CG | PHE | L | 98 | 8.577 | −29.066 | 17.054 | 1.00 | 25.88 | C |
| ATOM | 2450 | CD1 | PHE | L | 98 | 8.496 | −27.674 | 16.960 | 1.00 | 25.92 | C |
| ATOM | 2451 | CE1 | PHE | L | 98 | 9.528 | −26.859 | 17.426 | 1.00 | 25.45 | C |
| ATOM | 2452 | CZ | PHE | L | 98 | 10.672 | −27.424 | 17.986 | 1.00 | 23.96 | C |
| ATOM | 2453 | CE2 | PHE | L | 98 | 10.770 | −28.813 | 18.097 | 1.00 | 25.25 | C |
| ATOM | 2454 | CD2 | PHE | L | 98 | 9.706 | −29.631 | 17.636 | 1.00 | 26.23 | C |
| ATOM | 2455 | C | PHE | L | 98 | 6.613 | −31.606 | 14.897 | 1.00 | 26.13 | C |
| ATOM | 2456 | O | PHE | L | 98 | 5.559 | −31.281 | 14.394 | 1.00 | 25.23 | O |
| ATOM | 2457 | N | GLY | L | 99 | 6.905 | −32.866 | 15.210 | 1.00 | 27.01 | N |
| ATOM | 2458 | CA | GLY | L | 99 | 5.887 | −33.912 | 15.282 | 1.00 | 28.71 | C |
| ATOM | 2459 | C | GLY | L | 99 | 4.810 | −33.585 | 16.315 | 1.00 | 29.65 | C |
| ATOM | 2460 | O | GLY | L | 99 | 4.942 | −32.622 | 17.071 | 1.00 | 29.42 | O |
| ATOM | 2461 | N | GLN | L | 100 | 3.743 | −34.386 | 16.360 | 1.00 | 30.72 | N |
| ATOM | 2462 | CA | GLN | L | 100 | 2.634 | −34.119 | 17.304 | 1.00 | 31.97 | C |
| ATOM | 2463 | CB | GLN | L | 100 | 1.370 | −34.920 | 16.945 | 1.00 | 32.50 | C |
| ATOM | 2464 | CG | GLN | L | 100 | 0.988 | −34.841 | 15.494 | 1.00 | 36.12 | C |
| ATOM | 2465 | CD | GLN | L | 100 | 1.511 | −36.025 | 14.703 | 1.00 | 39.28 | C |
| ATOM | 2466 | OE1 | GLN | L | 100 | 2.718 | −36.340 | 14.717 | 1.00 | 37.49 | O |
| ATOM | 2467 | NE2 | GLN | L | 100 | 0.596 | −36.703 | 14.007 | 1.00 | 39.09 | N |
| ATOM | 2468 | C | GLN | L | 100 | 3.008 | −34.448 | 18.737 | 1.00 | 31.49 | C |
| ATOM | 2469 | O | GLN | L | 100 | 2.437 | −33.893 | 19.679 | 1.00 | 31.26 | O |
| ATOM | 2470 | N | GLY | L | 101 | 3.963 | −35.355 | 18.897 | 1.00 | 31.07 | N |
| ATOM | 2471 | CA | GLY | L | 101 | 4.325 | −35.845 | 20.213 | 1.00 | 30.93 | C |
| ATOM | 2472 | C | GLY | L | 101 | 3.865 | −37.269 | 20.406 | 1.00 | 30.64 | C |
| ATOM | 2473 | O | GLY | L | 101 | 2.819 | −37.664 | 19.907 | 1.00 | 30.67 | O |
| ATOM | 2474 | N | THR | L | 102 | 4.669 | −38.050 | 21.116 | 1.00 | 30.32 | N |
| ATOM | 2475 | CA | THR | L | 102 | 4.308 | −39.409 | 21.456 | 1.00 | 30.21 | C |
| ATOM | 2476 | CB | THR | L | 102 | 5.250 | −40.433 | 20.763 | 1.00 | 29.96 | C |
| ATOM | 2477 | OG1 | THR | L | 102 | 5.073 | −40.350 | 19.345 | 1.00 | 29.12 | O |
| ATOM | 2478 | CG2 | THR | L | 102 | 4.940 | −41.842 | 21.209 | 1.00 | 29.80 | C |
| ATOM | 2479 | C | THR | L | 102 | 4.387 | −39.503 | 22.977 | 1.00 | 30.78 | C |
| ATOM | 2480 | O | THR | L | 102 | 5.440 | −39.255 | 23.568 | 1.00 | 29.90 | O |
| ATOM | 2481 | N | LYS | L | 103 | 3.262 | −39.817 | 23.612 | 1.00 | 31.91 | N |
| ATOM | 2482 | CA | LYS | L | 103 | 3.227 | −39.895 | 25.081 | 1.00 | 32.81 | C |
| ATOM | 2483 | CB | LYS | L | 103 | 1.839 | −39.514 | 25.639 | 1.00 | 33.30 | C |
| ATOM | 2484 | CG | LYS | L | 103 | 1.712 | −39.480 | 27.188 | 1.00 | 35.72 | C |
| ATOM | 2485 | CD | LYS | L | 103 | 2.838 | −38.674 | 27.825 | 1.00 | 41.38 | C |
| ATOM | 2486 | CE | LYS | L | 103 | 2.753 | −38.649 | 29.360 | 1.00 | 44.96 | C |
| ATOM | 2487 | NZ | LYS | L | 103 | 3.746 | −39.574 | 29.988 | 1.00 | 44.46 | N |
| ATOM | 2488 | C | LYS | L | 103 | 3.642 | −41.303 | 25.489 | 1.00 | 32.68 | C |
| ATOM | 2489 | O | LYS | L | 103 | 2.978 | −42.276 | 25.138 | 1.00 | 32.58 | O |
| ATOM | 2490 | N | LEU | L | 104 | 4.776 | −41.403 | 26.175 | 1.00 | 32.61 | N |
| ATOM | 2491 | CA | LEU | L | 104 | 5.196 | −42.656 | 26.753 | 1.00 | 33.47 | C |
| ATOM | 2492 | CB | LEU | L | 104 | 6.719 | −42.802 | 26.690 | 1.00 | 32.82 | C |
| ATOM | 2493 | CG | LEU | L | 104 | 7.296 | −44.179 | 27.033 | 1.00 | 33.96 | C |
| ATOM | 2494 | CD1 | LEU | L | 104 | 8.560 | −44.427 | 26.252 | 1.00 | 32.42 | C |
| ATOM | 2495 | CD2 | LEU | L | 104 | 7.562 | −44.328 | 28.536 | 1.00 | 35.80 | C |
| ATOM | 2496 | C | LEU | L | 104 | 4.712 | −42.700 | 28.198 | 1.00 | 33.69 | C |
| ATOM | 2497 | O | LEU | L | 104 | 4.922 | −41.755 | 28.948 | 1.00 | 33.40 | O |
| ATOM | 2498 | N | GLU | L | 105 | 4.077 | −43.810 | 28.568 | 1.00 | 34.64 | N |
| ATOM | 2499 | CA | GLU | L | 105 | 3.601 | −44.036 | 29.932 | 1.00 | 35.26 | C |
| ATOM | 2500 | CB | GLU | L | 105 | 2.067 | −43.975 | 30.003 | 1.00 | 36.13 | C |
| ATOM | 2501 | CG | GLU | L | 105 | 1.361 | −45.110 | 29.210 | 1.00 | 39.20 | C |
| ATOM | 2502 | CD | GLU | L | 105 | −0.035 | −45.493 | 29.738 | 1.00 | 44.14 | C |
| ATOM | 2503 | OE1 | GLU | L | 105 | −0.710 | −44.647 | 30.356 | 1.00 | 46.98 | O |
| ATOM | 2504 | OE2 | GLU | L | 105 | −0.472 | −46.648 | 29.507 | 1.00 | 44.29 | O |
| ATOM | 2505 | C | GLU | L | 105 | 4.086 | −45.413 | 30.376 | 1.00 | 34.68 | C |
| ATOM | 2506 | O | GLU | L | 105 | 4.431 | −46.251 | 29.542 | 1.00 | 34.49 | O |
| ATOM | 2507 | N | ILE | L | 106 | 4.122 | −45.641 | 31.688 | 1.00 | 34.22 | N |
| ATOM | 2508 | CA | ILE | L | 106 | 4.518 | −46.949 | 32.223 | 1.00 | 33.60 | C |
| ATOM | 2509 | CB | ILE | L | 106 | 4.975 | −46.860 | 33.708 | 1.00 | 33.32 | C |
| ATOM | 2510 | CG1 | ILE | L | 106 | 6.189 | −45.925 | 33.864 | 1.00 | 32.87 | C |
| ATOM | 2511 | CD1 | ILE | L | 106 | 7.387 | −46.293 | 33.055 | 1.00 | 34.99 | C |
| ATOM | 2512 | CG2 | ILE | L | 106 | 5.209 | −48.271 | 34.293 | 1.00 | 33.53 | C |
| ATOM | 2513 | C | ILE | L | 106 | 3.373 | −47.933 | 32.129 | 1.00 | 33.24 | C |
| ATOM | 2514 | O | ILE | L | 106 | 2.261 | −47.633 | 32.550 | 1.00 | 32.88 | O |
| ATOM | 2515 | N | LYS | L | 107 | 3.657 | −49.117 | 31.604 | 1.00 | 33.60 | N |
| ATOM | 2516 | CA | LYS | L | 107 | 2.676 | −50.184 | 31.580 | 1.00 | 34.75 | C |
| ATOM | 2517 | CB | LYS | L | 107 | 2.886 | −51.097 | 30.374 | 1.00 | 34.61 | C |
| ATOM | 2518 | CG | LYS | L | 107 | 1.907 | −52.248 | 30.292 | 1.00 | 36.24 | C |
| ATOM | 2519 | CD | LYS | L | 107 | 2.138 | −53.066 | 29.017 | 1.00 | 36.96 | C |
| ATOM | 2520 | CE | LYS | L | 107 | 1.351 | −54.359 | 29.046 | 1.00 | 38.75 | C |
| ATOM | 2521 | NZ | LYS | L | 107 | 1.530 | −55.095 | 27.752 | 1.00 | 39.25 | N |
| ATOM | 2522 | C | LYS | L | 107 | 2.721 | −50.992 | 32.870 | 1.00 | 35.22 | C |
| ATOM | 2523 | O | LYS | L | 107 | 3.783 | −51.372 | 33.347 | 1.00 | 35.23 | O |
| ATOM | 2524 | N | ARG | L | 108 | 1.542 | −51.246 | 33.414 | 1.00 | 36.39 | N |
| ATOM | 2525 | CA | ARG | L | 108 | 1.375 | −52.050 | 34.615 | 1.00 | 37.87 | C |
| ATOM | 2526 | CB | ARG | L | 108 | 1.220 | −51.127 | 35.820 | 1.00 | 37.65 | C |
| ATOM | 2527 | CG | ARG | L | 108 | 0.107 | −50.092 | 35.684 | 1.00 | 38.37 | C |
| ATOM | 2528 | CD | ARG | L | 108 | −0.427 | −49.738 | 37.031 | 1.00 | 38.41 | C |
| ATOM | 2529 | NE | ARG | L | 108 | −1.111 | −50.881 | 37.638 | 1.00 | 40.85 | N |
| ATOM | 2530 | CZ | ARG | L | 108 | −1.155 | −51.125 | 38.944 | 1.00 | 39.67 | C |
| ATOM | 2531 | NH1 | ARG | L | 108 | −1.826 | −52.174 | 39.381 | 1.00 | 41.17 | N |
| ATOM | 2532 | NH2 | ARG | L | 108 | −0.545 | −50.322 | 39.809 | 1.00 | 38.71 | N |
| ATOM | 2533 | C | ARG | L | 108 | 0.138 | −52.942 | 34.407 | 1.00 | 38.66 | C |
| ATOM | 2534 | O | ARG | L | 108 | −0.533 | −52.818 | 33.383 | 1.00 | 38.39 | O |
| ATOM | 2535 | N | THR | L | 109 | −0.169 | −53.845 | 35.336 | 1.00 | 39.90 | N |
| ATOM | 2536 | CA | THR | L | 109 | −1.373 | −54.681 | 35.175 | 1.00 | 41.62 | C |
| ATOM | 2537 | CB | THR | L | 109 | −1.444 | −55.883 | 36.177 | 1.00 | 41.88 | C |
| ATOM | 2538 | OG1 | THR | L | 109 | −1.434 | −55.379 | 37.511 | 1.00 | 43.88 | O |
| ATOM | 2539 | CG2 | THR | L | 109 | −0.266 | −56.843 | 35.989 | 1.00 | 42.53 | C |
| ATOM | 2540 | C | THR | L | 109 | −2.639 | −53.835 | 35.322 | 1.00 | 42.09 | C |
| ATOM | 2541 | O | THR | L | 109 | −2.605 | −52.744 | 35.910 | 1.00 | 42.56 | O |
| ATOM | 2542 | N | VAL | L | 110 | −3.742 | −54.338 | 34.769 | 1.00 | 42.78 | N |
| ATOM | 2543 | CA | VAL | L | 110 | −5.044 | −53.709 | 34.917 | 1.00 | 43.67 | C |
| ATOM | 2544 | CB | VAL | L | 110 | −6.147 | −54.493 | 34.148 | 1.00 | 43.77 | C |
| ATOM | 2545 | CG1 | VAL | L | 110 | −7.509 | −53.952 | 34.463 | 1.00 | 44.13 | C |
| ATOM | 2546 | CG2 | VAL | L | 110 | −5.923 | −54.419 | 32.635 | 1.00 | 44.08 | C |
| ATOM | 2547 | C | VAL | L | 110 | −5.356 | −53.578 | 36.422 | 1.00 | 44.23 | C |
| ATOM | 2548 | O | VAL | L | 110 | −5.079 | −54.493 | 37.203 | 1.00 | 44.10 | O |
| ATOM | 2549 | N | ALA | L | 111 | −5.848 | −52.405 | 36.827 | 1.00 | 44.34 | N |
| ATOM | 2550 | CA | ALA | L | 111 | −6.294 | −52.181 | 38.203 | 1.00 | 44.34 | C |
| ATOM | 2551 | CB | ALA | L | 111 | −5.250 | −51.402 | 38.993 | 1.00 | 44.08 | C |
| ATOM | 2552 | C | ALA | L | 111 | −7.615 | −51.437 | 38.177 | 1.00 | 44.53 | C |
| ATOM | 2553 | O | ALA | L | 111 | −7.675 | −50.316 | 37.683 | 1.00 | 44.68 | O |
| ATOM | 2554 | N | ALA | L | 112 | −8.672 | −52.082 | 38.679 | 1.00 | 44.81 | N |
| ATOM | 2555 | CA | ALA | L | 112 | −10.010 | −51.476 | 38.805 | 1.00 | 44.81 | C |
| ATOM | 2556 | CB | ALA | L | 112 | −10.982 | −52.455 | 39.494 | 1.00 | 44.67 | C |
| ATOM | 2557 | C | ALA | L | 112 | −9.986 | −50.154 | 39.576 | 1.00 | 44.63 | C |
| ATOM | 2558 | O | ALA | L | 112 | −9.245 | −50.030 | 40.550 | 1.00 | 44.63 | O |
| ATOM | 2559 | N | PRO | L | 113 | −10.807 | −49.170 | 39.153 | 1.00 | 44.66 | N |
| ATOM | 2560 | CA | PRO | L | 113 | −10.907 | −47.930 | 39.920 | 1.00 | 45.09 | C |
| ATOM | 2561 | CB | PRO | L | 113 | −11.565 | −46.964 | 38.935 | 1.00 | 44.75 | C |
| ATOM | 2562 | CG | PRO | L | 113 | −12.354 | −47.812 | 38.024 | 1.00 | 44.69 | C |
| ATOM | 2563 | CD | PRO | L | 113 | −11.703 | −49.170 | 37.982 | 1.00 | 44.79 | C |
| ATOM | 2564 | C | PRO | L | 113 | −11.785 | −48.071 | 41.156 | 1.00 | 45.36 | C |
| ATOM | 2565 | O | PRO | L | 113 | −12.799 | −48.752 | 41.099 | 1.00 | 45.52 | O |
| ATOM | 2566 | N | SER | L | 114 | −11.377 | −47.449 | 42.260 | 1.00 | 45.98 | N |
| ATOM | 2567 | CA | SER | L | 114 | −12.279 | −47.180 | 43.380 | 1.00 | 46.85 | C |
| ATOM | 2568 | CB | SER | L | 114 | −11.504 | −46.835 | 44.655 | 1.00 | 46.86 | C |
| ATOM | 2569 | OG | SER | L | 114 | −10.862 | −47.977 | 45.173 | 1.00 | 49.57 | O |
| ATOM | 2570 | C | SER | L | 114 | −13.132 | −45.984 | 42.996 | 1.00 | 46.53 | C |
| ATOM | 2571 | O | SER | L | 114 | −12.594 | −44.935 | 42.625 | 1.00 | 46.63 | O |
| ATOM | 2572 | N | VAL | L | 115 | −14.449 | −46.149 | 43.103 | 1.00 | 46.34 | N |
| ATOM | 2573 | CA | VAL | L | 115 | −15.420 | −45.143 | 42.693 | 1.00 | 45.81 | C |
| ATOM | 2574 | CB | VAL | L | 115 | −16.559 | −45.777 | 41.870 | 1.00 | 45.72 | C |
| ATOM | 2575 | CG1 | VAL | L | 115 | −17.425 | −44.709 | 41.238 | 1.00 | 45.10 | C |
| ATOM | 2576 | CG2 | VAL | L | 115 | −16.002 | −46.693 | 40.799 | 1.00 | 45.43 | C |
| ATOM | 2577 | C | VAL | L | 115 | −16.028 | −44.452 | 43.910 | 1.00 | 46.33 | C |
| ATOM | 2578 | O | VAL | L | 115 | −16.365 | −45.106 | 44.896 | 1.00 | 46.06 | O |
| ATOM | 2579 | N | PHE | L | 116 | −16.177 | −43.131 | 43.826 | 1.00 | 46.54 | N |
| ATOM | 2580 | CA | PHE | L | 116 | −16.835 | −42.341 | 44.862 | 1.00 | 47.07 | C |
| ATOM | 2581 | CB | PHE | L | 116 | −15.798 | −41.622 | 45.746 | 1.00 | 47.12 | C |
| ATOM | 2582 | CG | PHE | L | 116 | −14.802 | −42.548 | 46.423 | 1.00 | 48.86 | C |
| ATOM | 2583 | CD1 | PHE | L | 116 | −15.053 | −43.053 | 47.708 | 1.00 | 50.53 | C |
| ATOM | 2584 | CE1 | PHE | L | 116 | −14.130 | −43.910 | 48.351 | 1.00 | 50.83 | C |
| ATOM | 2585 | CZ | PHE | L | 116 | −12.942 | −44.259 | 47.705 | 1.00 | 50.64 | C |
| ATOM | 2586 | CE2 | PHE | L | 116 | −12.675 | −43.750 | 46.417 | 1.00 | 51.18 | C |
| ATOM | 2587 | CD2 | PHE | L | 116 | −13.605 | −42.895 | 45.790 | 1.00 | 49.84 | C |
| ATOM | 2588 | C | PHE | L | 116 | −17.758 | −41.307 | 44.203 | 1.00 | 47.36 | C |
| ATOM | 2589 | O | PHE | L | 116 | −17.356 | −40.618 | 43.265 | 1.00 | 47.16 | O |
| ATOM | 2590 | N | ILE | L | 117 | −18.993 | −41.204 | 44.692 | 1.00 | 47.77 | N |
| ATOM | 2591 | CA | ILE | L | 117 | −19.937 | −40.179 | 44.241 | 1.00 | 47.88 | C |
| ATOM | 2592 | CB | ILE | L | 117 | −21.283 | −40.791 | 43.743 | 1.00 | 47.65 | C |
| ATOM | 2593 | CG1 | ILE | L | 117 | −22.145 | −39.727 | 43.044 | 1.00 | 47.20 | C |
| ATOM | 2594 | CD1 | ILE | L | 117 | −23.349 | −40.288 | 42.260 | 1.00 | 44.65 | C |
| ATOM | 2595 | CG2 | ILE | L | 117 | −22.048 | −41.499 | 44.886 | 1.00 | 47.85 | C |
| ATOM | 2596 | C | ILE | L | 117 | −20.150 | −39.142 | 45.351 | 1.00 | 48.60 | C |
| ATOM | 2597 | O | ILE | L | 117 | −20.256 | −39.495 | 46.529 | 1.00 | 48.45 | O |
| ATOM | 2598 | N | PHE | L | 118 | −20.166 | −37.868 | 44.962 | 1.00 | 49.14 | N |
| ATOM | 2599 | CA | PHE | L | 118 | −20.344 | −36.756 | 45.885 | 1.00 | 49.84 | C |
| ATOM | 2600 | CB | PHE | L | 118 | −19.094 | −35.873 | 45.935 | 1.00 | 49.45 | C |
| ATOM | 2601 | CG | PHE | L | 118 | −17.833 | −36.604 | 46.292 | 1.00 | 49.51 | C |
| ATOM | 2602 | CD1 | PHE | L | 118 | −17.388 | −36.646 | 47.609 | 1.00 | 48.92 | C |
| ATOM | 2603 | CE1 | PHE | L | 118 | −16.202 | −37.307 | 47.945 | 1.00 | 48.73 | C |
| ATOM | 2604 | CZ | PHE | L | 118 | −15.460 | −37.935 | 46.956 | 1.00 | 48.48 | C |
| ATOM | 2605 | CE2 | PHE | L | 118 | −15.895 | −37.898 | 45.634 | 1.00 | 48.57 | C |
| ATOM | 2606 | CD2 | PHE | L | 118 | −17.072 | −37.229 | 45.310 | 1.00 | 48.66 | C |
| ATOM | 2607 | C | PHE | L | 118 | −21.509 | −35.892 | 45.417 | 1.00 | 50.72 | C |
| ATOM | 2608 | O | PHE | L | 118 | −21.490 | −35.387 | 44.288 | 1.00 | 50.57 | O |
| ATOM | 2609 | N | PRO | L | 119 | −22.515 | −35.695 | 46.288 | 1.00 | 51.57 | N |
| ATOM | 2610 | CA | PRO | L | 119 | −23.628 | −34.785 | 45.992 | 1.00 | 51.92 | C |
| ATOM | 2611 | CB | PRO | L | 119 | −24.622 | −35.059 | 47.139 | 1.00 | 52.41 | C |
| ATOM | 2612 | CG | PRO | L | 119 | −24.053 | −36.225 | 47.934 | 1.00 | 51.86 | C |
| ATOM | 2613 | CD | PRO | L | 119 | −22.593 | −36.225 | 47.663 | 1.00 | 51.69 | C |
| ATOM | 2614 | C | PRO | L | 119 | −23.163 | −33.326 | 46.035 | 1.00 | 52.54 | C |
| ATOM | 2615 | O | PRO | L | 119 | −22.079 | −33.043 | 46.577 | 1.00 | 52.78 | O |
| ATOM | 2616 | N | PRO | L | 120 | −23.954 | −32.398 | 45.461 | 1.00 | 52.96 | N |
| ATOM | 2617 | CA | PRO | L | 120 | −23.639 | −30.966 | 45.596 | 1.00 | 53.51 | C |
| ATOM | 2618 | CB | PRO | L | 120 | −24.622 | −30.293 | 44.629 | 1.00 | 53.17 | C |
| ATOM | 2619 | CG | PRO | L | 120 | −25.736 | −31.241 | 44.497 | 1.00 | 52.92 | C |
| ATOM | 2620 | CD | PRO | L | 120 | −25.134 | −32.621 | 44.606 | 1.00 | 52.88 | C |
| ATOM | 2621 | C | PRO | L | 120 | −23.875 | −30.445 | 47.012 | 1.00 | 54.28 | C |
| ATOM | 2622 | O | PRO | L | 120 | −24.802 | −30.893 | 47.676 | 1.00 | 54.49 | O |
| ATOM | 2623 | N | SER | L | 121 | −23.040 | −29.507 | 47.460 | 1.00 | 55.38 | N |
| ATOM | 2624 | CA | SER | L | 121 | −23.213 | −28.852 | 48.759 | 1.00 | 56.39 | C |
| ATOM | 2625 | CB | SER | L | 121 | −21.933 | −28.103 | 49.146 | 1.00 | 56.41 | C |
| ATOM | 2626 | OG | SER | L | 121 | −21.667 | −27.049 | 48.234 | 1.00 | 55.27 | O |
| ATOM | 2627 | C | SER | L | 121 | −24.392 | −27.873 | 48.738 | 1.00 | 57.31 | C |
| ATOM | 2628 | O | SER | L | 121 | −24.738 | −27.339 | 47.683 | 1.00 | 57.32 | O |
| ATOM | 2629 | N | ASP | L | 122 | −24.989 | −27.618 | 49.903 | 1.00 | 58.62 | N |
| ATOM | 2630 | CA | ASP | L | 122 | −26.121 | −26.678 | 49.995 | 1.00 | 59.75 | C |
| ATOM | 2631 | CB | ASP | L | 122 | −26.840 | −26.787 | 51.341 | 1.00 | 59.87 | C |
| ATOM | 2632 | CG | ASP | L | 122 | −26.941 | −28.209 | 51.829 | 1.00 | 59.73 | C |
| ATOM | 2633 | OD1 | ASP | L | 122 | −27.980 | −28.856 | 51.584 | 1.00 | 58.54 | O |
| ATOM | 2634 | OD2 | ASP | L | 122 | −25.960 | −28.679 | 52.446 | 1.00 | 61.68 | O |
| ATOM | 2635 | C | ASP | L | 122 | −25.670 | −25.242 | 49.760 | 1.00 | 60.60 | C |
| ATOM | 2636 | O | ASP | L | 122 | −26.428 | −24.430 | 49.221 | 1.00 | 61.00 | O |
| ATOM | 2637 | N | GLU | L | 123 | −24.437 | −24.937 | 50.168 | 1.00 | 61.46 | N |
| ATOM | 2638 | CA | GLU | L | 123 | −23.805 | −23.651 | 49.861 | 1.00 | 62.37 | C |
| ATOM | 2639 | CB | GLU | L | 123 | −22.378 | −23.596 | 50.425 | 1.00 | 62.35 | C |
| ATOM | 2640 | CG | GLU | L | 123 | −21.795 | −22.180 | 50.560 | 1.00 | 63.90 | C |
| ATOM | 2641 | CD | GLU | L | 123 | −20.292 | −22.159 | 50.876 | 1.00 | 65.49 | C |
| ATOM | 2642 | OE1 | GLU | L | 123 | −19.670 | −21.081 | 50.712 | 1.00 | 65.61 | O |
| ATOM | 2643 | OE2 | GLU | L | 123 | −19.730 | −23.207 | 51.285 | 1.00 | 66.17 | O |
| ATOM | 2644 | C | GLU | L | 123 | −23.804 | −23.393 | 48.347 | 1.00 | 62.81 | C |
| ATOM | 2645 | O | GLU | L | 123 | −24.067 | −22.274 | 47.906 | 1.00 | 62.95 | O |
| ATOM | 2646 | N | GLN | L | 124 | −23.524 | −24.434 | 47.559 | 1.00 | 63.24 | N |
| ATOM | 2647 | CA | GLN | L | 124 | −23.594 | −24.334 | 46.103 | 1.00 | 63.64 | C |
| ATOM | 2648 | CB | GLN | L | 124 | −22.807 | −25.453 | 45.422 | 1.00 | 63.35 | C |
| ATOM | 2649 | CG | GLN | L | 124 | −22.367 | −25.082 | 44.008 | 1.00 | 62.47 | C |
| ATOM | 2650 | CD | GLN | L | 124 | −22.180 | −26.271 | 43.090 | 1.00 | 61.06 | C |
| ATOM | 2651 | OE1 | GLN | L | 124 | −22.216 | −27.426 | 43.518 | 1.00 | 59.87 | O |
| ATOM | 2652 | NE2 | GLN | L | 124 | −21.981 | −25.991 | 41.808 | 1.00 | 60.70 | N |
| ATOM | 2653 | C | GLN | L | 124 | −25.039 | −24.319 | 45.585 | 1.00 | 64.13 | C |
| ATOM | 2654 | O | GLN | L | 124 | −25.317 | −23.765 | 44.520 | 1.00 | 64.41 | O |
| ATOM | 2655 | N | LEU | L | 125 | −25.951 | −24.936 | 46.328 | 1.00 | 64.48 | N |
| ATOM | 2656 | CA | LEU | L | 125 | −27.364 | −24.917 | 45.949 | 1.00 | 65.06 | C |
| ATOM | 2657 | CB | LEU | L | 125 | −28.149 | −25.965 | 46.731 | 1.00 | 65.01 | C |
| ATOM | 2658 | CG | LEU | L | 125 | −27.956 | −27.389 | 46.213 | 1.00 | 65.13 | C |
| ATOM | 2659 | CD1 | LEU | L | 125 | −28.808 | −27.637 | 44.979 | 1.00 | 64.84 | C |
| ATOM | 2660 | CD2 | LEU | L | 125 | −28.278 | −28.393 | 47.305 | 1.00 | 65.42 | C |
| ATOM | 2661 | C | LEU | L | 125 | −27.994 | −23.528 | 46.092 | 1.00 | 65.25 | C |
| ATOM | 2662 | O | LEU | L | 125 | −28.914 | −23.194 | 45.356 | 1.00 | 65.48 | O |
| ATOM | 2663 | N | LYS | L | 126 | −27.488 | −22.724 | 47.029 | 1.00 | 65.59 | N |
| ATOM | 2664 | CA | LYS | L | 126 | −27.843 | −21.297 | 47.104 | 1.00 | 65.97 | C |
| ATOM | 2665 | CB | LYS | L | 126 | −27.179 | −20.608 | 48.305 | 1.00 | 66.08 | C |
| ATOM | 2666 | CG | LYS | L | 126 | −27.601 | −21.070 | 49.700 | 1.00 | 66.77 | C |
| ATOM | 2667 | CD | LYS | L | 126 | −27.083 | −20.067 | 50.750 | 1.00 | 67.90 | C |
| ATOM | 2668 | CE | LYS | L | 126 | −26.749 | −20.722 | 52.101 | 1.00 | 68.38 | C |
| ATOM | 2669 | NZ | LYS | L | 126 | −27.959 | −21.244 | 52.808 | 1.00 | 68.99 | N |
| ATOM | 2670 | C | LYS | L | 126 | −27.416 | −20.547 | 45.834 | 1.00 | 65.88 | C |
| ATOM | 2671 | O | LYS | L | 126 | −27.953 | −19.481 | 45.526 | 1.00 | 66.25 | O |
| ATOM | 2672 | N | SER | L | 127 | −26.432 | −21.093 | 45.122 | 1.00 | 65.63 | N |
| ATOM | 2673 | CA | SER | L | 127 | −25.876 | −20.443 | 43.933 | 1.00 | 65.43 | C |
| ATOM | 2674 | CB | SER | L | 127 | −24.525 | −21.060 | 43.544 | 1.00 | 65.56 | C |
| ATOM | 2675 | OG | SER | L | 127 | −23.543 | −20.851 | 44.550 | 1.00 | 66.35 | O |
| ATOM | 2676 | C | SER | L | 127 | −26.825 | −20.529 | 42.750 | 1.00 | 64.89 | C |
| ATOM | 2677 | O | SER | L | 127 | −26.790 | −19.676 | 41.864 | 1.00 | 65.09 | O |
| ATOM | 2678 | N | GLY | L | 128 | −27.669 | −21.560 | 42.744 | 1.00 | 64.22 | N |
| ATOM | 2679 | CA | GLY | L | 128 | −28.542 | −21.839 | 41.607 | 1.00 | 62.97 | C |
| ATOM | 2680 | C | GLY | L | 128 | −27.922 | −22.820 | 40.626 | 1.00 | 62.07 | C |
| ATOM | 2681 | O | GLY | L | 128 | −28.324 | −22.882 | 39.463 | 1.00 | 62.55 | O |
| ATOM | 2682 | N | THR | L | 129 | −26.943 | −23.590 | 41.095 | 1.00 | 60.78 | N |
| ATOM | 2683 | CA | THR | L | 129 | −26.268 | −24.603 | 40.280 | 1.00 | 59.11 | C |
| ATOM | 2684 | CB | THR | L | 129 | −24.973 | −24.048 | 39.653 | 1.00 | 59.16 | C |
| ATOM | 2685 | OG1 | THR | L | 129 | −25.282 | −22.893 | 38.871 | 1.00 | 59.64 | O |
| ATOM | 2686 | CG2 | THR | L | 129 | −24.313 | −25.075 | 38.750 | 1.00 | 59.49 | C |
| ATOM | 2687 | C | THR | L | 129 | −25.921 | −25.813 | 41.142 | 1.00 | 57.79 | C |
| ATOM | 2688 | O | THR | L | 129 | −25.599 | −25.671 | 42.325 | 1.00 | 57.82 | O |
| ATOM | 2689 | N | ALA | L | 130 | −25.989 | −26.997 | 40.545 | 1.00 | 55.82 | N |
| ATOM | 2690 | CA | ALA | L | 130 | −25.602 | −28.214 | 41.228 | 1.00 | 54.16 | C |
| ATOM | 2691 | CB | ALA | L | 130 | −26.801 | −29.112 | 41.417 | 1.00 | 54.19 | C |
| ATOM | 2692 | C | ALA | L | 130 | −24.492 | −28.944 | 40.477 | 1.00 | 52.96 | C |
| ATOM | 2693 | O | ALA | L | 130 | −24.669 | −29.351 | 39.330 | 1.00 | 52.59 | O |
| ATOM | 2694 | N | SER | L | 131 | −23.341 | −29.088 | 41.126 | 1.00 | 51.55 | N |
| ATOM | 2695 | CA | SER | L | 131 | −22.272 | −29.937 | 40.605 | 1.00 | 50.60 | C |
| ATOM | 2696 | CB | SER | L | 131 | −20.920 | −29.248 | 40.709 | 1.00 | 50.51 | C |
| ATOM | 2697 | OG | SER | L | 131 | −20.906 | −28.030 | 39.994 | 1.00 | 50.49 | O |
| ATOM | 2698 | C | SER | L | 131 | −22.237 | −31.278 | 41.337 | 1.00 | 49.73 | C |
| ATOM | 2699 | O | SER | L | 131 | −22.072 | −31.340 | 42.557 | 1.00 | 49.42 | O |
| ATOM | 2700 | N | VAL | L | 132 | −22.411 | −32.346 | 40.574 | 1.00 | 49.10 | N |
| ATOM | 2701 | CA | VAL | L | 132 | −22.309 | −33.705 | 41.087 | 1.00 | 48.11 | C |
| ATOM | 2702 | CB | VAL | L | 132 | −23.510 | −34.566 | 40.655 | 1.00 | 47.99 | C |
| ATOM | 2703 | CG1 | VAL | L | 132 | −23.609 | −35.804 | 41.540 | 1.00 | 48.28 | C |
| ATOM | 2704 | CG2 | VAL | L | 132 | −24.791 | −33.765 | 40.735 | 1.00 | 47.70 | C |
| ATOM | 2705 | C | VAL | L | 132 | −21.015 | −34.332 | 40.574 | 1.00 | 47.83 | C |
| ATOM | 2706 | O | VAL | L | 132 | −20.779 | −34.380 | 39.364 | 1.00 | 47.66 | O |
| ATOM | 2707 | N | VAL | L | 133 | −20.186 | −34.812 | 41.495 | 1.00 | 47.01 | N |
| ATOM | 2708 | CA | VAL | L | 133 | −18.852 | −35.287 | 41.160 | 1.00 | 46.69 | C |
| ATOM | 2709 | CB | VAL | L | 133 | −17.753 | −34.500 | 41.930 | 1.00 | 46.89 | C |
| ATOM | 2710 | CG1 | VAL | L | 133 | −16.348 | −35.041 | 41.625 | 1.00 | 46.13 | C |
| ATOM | 2711 | CG2 | VAL | L | 133 | −17.841 | −32.988 | 41.612 | 1.00 | 46.66 | C |
| ATOM | 2712 | C | VAL | L | 133 | −18.702 | −36.791 | 41.386 | 1.00 | 46.66 | C |
| ATOM | 2713 | O | VAL | L | 133 | −19.137 | −37.344 | 42.407 | 1.00 | 46.53 | O |
| ATOM | 2714 | N | CYS | L | 134 | −18.109 | −37.439 | 40.395 | 1.00 | 46.18 | N |
| ATOM | 2715 | CA | CYS | L | 134 | −17.767 | −38.840 | 40.468 | 1.00 | 46.42 | C |
| ATOM | 2716 | CB | CYS | L | 134 | −18.464 | −39.605 | 39.362 | 1.00 | 46.71 | C |
| ATOM | 2717 | SG | CYS | L | 134 | −18.253 | −41.372 | 39.489 | 1.00 | 49.16 | S |
| ATOM | 2718 | C | CYS | L | 134 | −16.258 | −38.976 | 40.327 | 1.00 | 46.03 | C |
| ATOM | 2719 | O | CYS | L | 134 | −15.643 | −38.374 | 39.427 | 1.00 | 45.95 | O |
| ATOM | 2720 | N | LEU | L | 135 | −15.673 | −39.748 | 41.234 | 1.00 | 45.12 | N |
| ATOM | 2721 | CA | LEU | L | 135 | −14.248 | −39.964 | 41.263 | 1.00 | 45.10 | C |
| ATOM | 2722 | CB | LEU | L | 135 | −13.670 | −39.529 | 42.609 | 1.00 | 45.18 | C |
| ATOM | 2723 | CG | LEU | L | 135 | −12.221 | −39.911 | 42.935 | 1.00 | 45.85 | C |
| ATOM | 2724 | CD1 | LEU | L | 135 | −11.236 | −39.268 | 41.964 | 1.00 | 43.59 | C |
| ATOM | 2725 | CD2 | LEU | L | 135 | −11.907 | −39.501 | 44.370 | 1.00 | 46.14 | C |
| ATOM | 2726 | C | LEU | L | 135 | −13.899 | −41.420 | 40.996 | 1.00 | 44.98 | C |
| ATOM | 2727 | O | LEU | L | 135 | −14.435 | −42.335 | 41.648 | 1.00 | 44.83 | O |
| ATOM | 2728 | N | LEU | L | 136 | −13.006 | −41.608 | 40.030 | 1.00 | 44.00 | N |
| ATOM | 2729 | CA | LEU | L | 136 | −12.362 | −42.876 | 39.764 | 1.00 | 44.18 | C |
| ATOM | 2730 | CB | LEU | L | 136 | −12.383 | −43.177 | 38.267 | 1.00 | 43.89 | C |
| ATOM | 2731 | CG | LEU | L | 136 | −13.688 | −43.683 | 37.648 | 1.00 | 42.82 | C |
| ATOM | 2732 | CD1 | LEU | L | 136 | −14.851 | −42.695 | 37.821 | 1.00 | 43.62 | C |
| ATOM | 2733 | CD2 | LEU | L | 136 | −13.461 | −43.976 | 36.189 | 1.00 | 41.57 | C |
| ATOM | 2734 | C | LEU | L | 136 | −10.929 | −42.756 | 40.247 | 1.00 | 44.91 | C |
| ATOM | 2735 | O | LEU | L | 136 | −10.158 | −41.938 | 39.722 | 1.00 | 44.92 | O |
| ATOM | 2736 | N | ASN | L | 137 | −10.579 | −43.544 | 41.264 | 1.00 | 45.23 | N |
| ATOM | 2737 | CA | ASN | L | 137 | −9.288 | −43.401 | 41.923 | 1.00 | 45.79 | C |
| ATOM | 2738 | CB | ASN | L | 137 | −9.459 | −43.207 | 43.436 | 1.00 | 45.67 | C |
| ATOM | 2739 | CG | ASN | L | 137 | −8.332 | −42.396 | 44.048 | 1.00 | 46.98 | C |
| ATOM | 2740 | OD1 | ASN | L | 137 | −8.034 | −41.286 | 43.596 | 1.00 | 48.99 | O |
| ATOM | 2741 | ND2 | ASN | L | 137 | −7.703 | −42.937 | 45.086 | 1.00 | 47.95 | N |
| ATOM | 2742 | C | ASN | L | 137 | −8.339 | −44.565 | 41.653 | 1.00 | 46.14 | C |
| ATOM | 2743 | O | ASN | L | 137 | −8.717 | −45.736 | 41.766 | 1.00 | 46.27 | O |
| ATOM | 2744 | N | ASN | L | 138 | −7.110 | −44.215 | 41.295 | 1.00 | 46.59 | N |
| ATOM | 2745 | CA | ASN | L | 138 | −6.009 | −45.162 | 41.159 | 1.00 | 47.14 | C |
| ATOM | 2746 | CB | ASN | L | 138 | −5.493 | −45.582 | 42.538 | 1.00 | 47.33 | C |
| ATOM | 2747 | CG | ASN | L | 138 | −5.134 | −44.399 | 43.407 | 1.00 | 48.20 | C |
| ATOM | 2748 | OD1 | ASN | L | 138 | −4.752 | −43.332 | 42.916 | 1.00 | 47.55 | O |
| ATOM | 2749 | ND2 | ASN | L | 138 | −5.264 | −44.578 | 44.710 | 1.00 | 49.48 | N |
| ATOM | 2750 | C | ASN | L | 138 | −6.345 | −46.373 | 40.292 | 1.00 | 47.08 | C |
| ATOM | 2751 | O | ASN | L | 138 | −6.309 | −47.522 | 40.753 | 1.00 | 46.56 | O |
| ATOM | 2752 | N | PHE | L | 139 | −6.677 | −46.097 | 39.032 | 1.00 | 46.91 | N |
| ATOM | 2753 | CA | PHE | L | 139 | −6.966 | −47.146 | 38.074 | 1.00 | 47.09 | C |
| ATOM | 2754 | CB | PHE | L | 139 | −8.391 | −47.035 | 37.544 | 1.00 | 47.06 | C |
| ATOM | 2755 | CG | PHE | L | 139 | −8.646 | −45.788 | 36.759 | 1.00 | 48.33 | C |
| ATOM | 2756 | CD1 | PHE | L | 139 | −8.904 | −44.582 | 37.403 | 1.00 | 48.41 | C |
| ATOM | 2757 | CE1 | PHE | L | 139 | −9.125 | −43.420 | 36.670 | 1.00 | 49.33 | C |
| ATOM | 2758 | CZ | PHE | L | 139 | −9.096 | −43.464 | 35.293 | 1.00 | 48.90 | C |
| ATOM | 2759 | CE2 | PHE | L | 139 | −8.841 | −44.666 | 34.643 | 1.00 | 48.65 | C |
| ATOM | 2760 | CD2 | PHE | L | 139 | −8.623 | −45.815 | 35.374 | 1.00 | 48.06 | C |
| ATOM | 2761 | C | PHE | L | 139 | −5.971 | −47.148 | 36.925 | 1.00 | 46.95 | C |
| ATOM | 2762 | O | PHE | L | 139 | −5.307 | −46.136 | 36.659 | 1.00 | 46.92 | O |
| ATOM | 2763 | N | TYR | L | 140 | −5.868 | −48.303 | 36.272 | 1.00 | 46.46 | N |
| ATOM | 2764 | CA | TYR | L | 140 | −5.092 | −48.467 | 35.058 | 1.00 | 46.56 | C |
| ATOM | 2765 | CB | TYR | L | 140 | −3.642 | −48.889 | 35.362 | 1.00 | 45.75 | C |
| ATOM | 2766 | CG | TYR | L | 140 | −2.781 | −48.904 | 34.121 | 1.00 | 42.26 | C |
| ATOM | 2767 | CD1 | TYR | L | 140 | −2.623 | −50.075 | 33.376 | 1.00 | 38.98 | C |
| ATOM | 2768 | CE1 | TYR | L | 140 | −1.855 | −50.093 | 32.219 | 1.00 | 36.77 | C |
| ATOM | 2769 | CZ | TYR | L | 140 | −1.245 | −48.929 | 31.776 | 1.00 | 35.50 | C |
| ATOM | 2770 | OH | TYR | L | 140 | −0.491 | −48.975 | 30.622 | 1.00 | 34.84 | O |
| ATOM | 2771 | CE2 | TYR | L | 140 | −1.383 | −47.747 | 32.488 | 1.00 | 36.25 | C |
| ATOM | 2772 | CD2 | TYR | L | 140 | −2.158 | −47.739 | 33.662 | 1.00 | 38.67 | C |
| ATOM | 2773 | C | TYR | L | 140 | −5.763 | −49.523 | 34.188 | 1.00 | 47.63 | C |
| ATOM | 2774 | O | TYR | L | 140 | −6.310 | −50.496 | 34.720 | 1.00 | 48.50 | O |
| ATOM | 2775 | N | PRO | L | 141 | −5.750 | −49.344 | 32.854 | 1.00 | 48.05 | N |
| ATOM | 2776 | CA | PRO | L | 141 | −5.311 | −48.213 | 32.031 | 1.00 | 48.33 | C |
| ATOM | 2777 | CB | PRO | L | 141 | −5.384 | −48.780 | 30.613 | 1.00 | 48.08 | C |
| ATOM | 2778 | CG | PRO | L | 141 | −6.403 | −49.847 | 30.713 | 1.00 | 47.97 | C |
| ATOM | 2779 | CD | PRO | L | 141 | −6.121 | −50.492 | 32.012 | 1.00 | 47.92 | C |
| ATOM | 2780 | C | PRO | L | 141 | −6.247 | −47.012 | 32.136 | 1.00 | 48.96 | C |
| ATOM | 2781 | O | PRO | L | 141 | −7.334 | −47.122 | 32.717 | 1.00 | 48.58 | O |
| ATOM | 2782 | N | ARG | L | 142 | −5.807 | −45.898 | 31.551 | 1.00 | 49.63 | N |
| ATOM | 2783 | CA | ARG | L | 142 | −6.521 | −44.619 | 31.519 | 1.00 | 50.83 | C |
| ATOM | 2784 | CB | ARG | L | 142 | −5.779 | −43.681 | 30.565 | 1.00 | 50.98 | C |
| ATOM | 2785 | CG | ARG | L | 142 | −6.078 | −42.193 | 30.701 | 1.00 | 52.36 | C |
| ATOM | 2786 | CD | ARG | L | 142 | −5.129 | −41.392 | 29.786 | 1.00 | 55.20 | C |
| ATOM | 2787 | NE | ARG | L | 142 | −5.448 | −39.960 | 29.788 | 1.00 | 58.94 | N |
| ATOM | 2788 | CZ | ARG | L | 142 | −6.243 | −39.365 | 28.897 | 1.00 | 59.91 | C |
| ATOM | 2789 | NH1 | ARG | L | 142 | −6.799 | −40.071 | 27.916 | 1.00 | 60.85 | N |
| ATOM | 2790 | NH2 | ARG | L | 142 | −6.491 | −38.063 | 28.987 | 1.00 | 59.36 | N |
| ATOM | 2791 | C | ARG | L | 142 | −7.970 | −44.766 | 31.055 | 1.00 | 51.24 | C |
| ATOM | 2792 | O | ARG | L | 142 | −8.880 | −44.131 | 31.597 | 1.00 | 51.24 | O |
| ATOM | 2793 | N | GLU | L | 143 | −8.163 | −45.607 | 30.044 | 1.00 | 51.96 | N |
| ATOM | 2794 | CA | GLU | L | 143 | −9.454 | −45.783 | 29.379 | 1.00 | 52.80 | C |
| ATOM | 2795 | CB | GLU | L | 143 | −9.300 | −46.732 | 28.185 | 1.00 | 52.88 | C |
| ATOM | 2796 | CG | GLU | L | 143 | −10.595 | −46.976 | 27.408 | 1.00 | 55.35 | C |
| ATOM | 2797 | CD | GLU | L | 143 | −10.509 | −48.168 | 26.455 | 1.00 | 57.52 | C |
| ATOM | 2798 | OE1 | GLU | L | 143 | −9.770 | −49.138 | 26.763 | 1.00 | 58.07 | O |
| ATOM | 2799 | OE2 | GLU | L | 143 | −11.183 | −48.125 | 25.397 | 1.00 | 57.92 | O |
| ATOM | 2800 | C | GLU | L | 143 | −10.549 | −46.296 | 30.316 | 1.00 | 52.73 | C |
| ATOM | 2801 | O | GLU | L | 143 | −10.473 | −47.402 | 30.841 | 1.00 | 52.61 | O |
| ATOM | 2802 | N | ALA | L | 144 | −11.569 | −45.469 | 30.508 | 1.00 | 52.99 | N |
| ATOM | 2803 | CA | ALA | L | 144 | −12.744 | −45.834 | 31.282 | 1.00 | 52.94 | C |
| ATOM | 2804 | CB | ALA | L | 144 | −12.547 | −45.488 | 32.745 | 1.00 | 52.96 | C |
| ATOM | 2805 | C | ALA | L | 144 | −13.929 | −45.078 | 30.718 | 1.00 | 52.96 | C |
| ATOM | 2806 | O | ALA | L | 144 | −13.784 | −43.951 | 30.241 | 1.00 | 52.66 | O |
| ATOM | 2807 | N | LYS | L | 145 | −15.098 | −45.708 | 30.762 | 1.00 | 53.35 | N |
| ATOM | 2808 | CA | LYS | L | 145 | −16.348 | −45.049 | 30.386 | 1.00 | 53.72 | C |
| ATOM | 2809 | CB | LYS | L | 145 | −17.182 | −45.953 | 29.477 | 1.00 | 53.83 | C |
| ATOM | 2810 | CG | LYS | L | 145 | −18.304 | −45.238 | 28.729 | 1.00 | 54.75 | C |
| ATOM | 2811 | CD | LYS | L | 145 | −19.168 | −46.243 | 27.969 | 1.00 | 55.84 | C |
| ATOM | 2812 | CE | LYS | L | 145 | −20.180 | −45.556 | 27.058 | 1.00 | 56.84 | C |
| ATOM | 2813 | NZ | LYS | L | 145 | −20.927 | −46.552 | 26.227 | 1.00 | 57.57 | N |
| ATOM | 2814 | C | LYS | L | 145 | −17.139 | −44.676 | 31.641 | 1.00 | 53.63 | C |
| ATOM | 2815 | O | LYS | L | 145 | −17.436 | −45.531 | 32.486 | 1.00 | 53.70 | O |
| ATOM | 2816 | N | VAL | L | 146 | −17.461 | −43.393 | 31.757 | 1.00 | 53.75 | N |
| ATOM | 2817 | CA | VAL | L | 146 | −18.283 | −42.890 | 32.850 | 1.00 | 53.85 | C |
| ATOM | 2818 | CB | VAL | L | 146 | −17.591 | −41.728 | 33.599 | 1.00 | 53.78 | C |
| ATOM | 2819 | CG1 | VAL | L | 146 | −18.456 | −41.237 | 34.756 | 1.00 | 53.28 | C |
| ATOM | 2820 | CG2 | VAL | L | 146 | −16.215 | −42.150 | 34.099 | 1.00 | 53.27 | C |
| ATOM | 2821 | C | VAL | L | 146 | −19.630 | −42.434 | 32.293 | 1.00 | 54.22 | C |
| ATOM | 2822 | O | VAL | L | 146 | −19.705 | −41.458 | 31.557 | 1.00 | 54.48 | O |
| ATOM | 2823 | N | GLN | L | 147 | −20.692 | −43.155 | 32.633 | 1.00 | 54.51 | N |
| ATOM | 2824 | CA | GLN | L | 147 | −22.036 | −42.766 | 32.227 | 1.00 | 54.77 | C |
| ATOM | 2825 | CB | GLN | L | 147 | −22.801 | −43.956 | 31.645 | 1.00 | 54.73 | C |
| ATOM | 2826 | CG | GLN | L | 147 | −22.232 | −44.502 | 30.336 | 1.00 | 55.51 | C |
| ATOM | 2827 | CD | GLN | L | 147 | −23.221 | −45.386 | 29.569 | 1.00 | 55.60 | C |
| ATOM | 2828 | OE1 | GLN | L | 147 | −23.485 | −45.155 | 28.387 | 1.00 | 54.64 | O |
| ATOM | 2829 | NE2 | GLN | L | 147 | −23.776 | −46.393 | 30.246 | 1.00 | 55.73 | N |
| ATOM | 2830 | C | GLN | L | 147 | −22.797 | −42.187 | 33.410 | 1.00 | 55.00 | C |
| ATOM | 2831 | O | GLN | L | 147 | −22.744 | −42.726 | 34.521 | 1.00 | 54.99 | O |
| ATOM | 2832 | N | TRP | L | 148 | −23.500 | −41.083 | 33.170 | 1.00 | 55.27 | N |
| ATOM | 2833 | CA | TRP | L | 148 | −24.352 | −40.484 | 34.189 | 1.00 | 55.56 | C |
| ATOM | 2834 | CB | TRP | L | 148 | −24.237 | −38.955 | 34.191 | 1.00 | 54.96 | C |
| ATOM | 2835 | CG | TRP | L | 148 | −23.030 | −38.427 | 34.918 | 1.00 | 54.17 | C |
| ATOM | 2836 | CD1 | TRP | L | 148 | −21.867 | −37.982 | 34.358 | 1.00 | 53.04 | C |
| ATOM | 2837 | NE1 | TRP | L | 148 | −20.992 | −37.575 | 35.340 | 1.00 | 53.10 | N |
| ATOM | 2838 | CE2 | TRP | L | 148 | −21.581 | −37.746 | 36.564 | 1.00 | 52.34 | C |
| ATOM | 2839 | CD2 | TRP | L | 148 | −22.868 | −38.286 | 36.342 | 1.00 | 53.08 | C |
| ATOM | 2840 | CE3 | TRP | L | 148 | −23.685 | −38.563 | 37.449 | 1.00 | 52.10 | C |
| ATOM | 2841 | CZ3 | TRP | L | 148 | −23.194 | −38.296 | 38.724 | 1.00 | 51.56 | C |
| ATOM | 2842 | CH2 | TRP | L | 148 | −21.905 | −37.763 | 38.908 | 1.00 | 52.01 | C |
| ATOM | 2843 | CZ2 | TRP | L | 148 | −21.089 | −37.482 | 37.844 | 1.00 | 51.52 | C |
| ATOM | 2844 | C | TRP | L | 148 | −25.799 | −40.908 | 33.970 | 1.00 | 56.37 | C |
| ATOM | 2845 | O | TRP | L | 148 | −26.321 | −40.805 | 32.856 | 1.00 | 56.17 | O |
| ATOM | 2846 | N | LYS | L | 149 | −26.426 | −41.402 | 35.038 | 1.00 | 57.29 | N |
| ATOM | 2847 | CA | LYS | L | 149 | −27.831 | −41.806 | 35.009 | 1.00 | 58.23 | C |
| ATOM | 2848 | CB | LYS | L | 149 | −27.977 | −43.298 | 35.324 | 1.00 | 58.43 | C |
| ATOM | 2849 | CG | LYS | L | 149 | −27.319 | −44.236 | 34.314 | 1.00 | 60.02 | C |
| ATOM | 2850 | CD | LYS | L | 149 | −28.009 | −45.603 | 34.296 | 1.00 | 61.10 | C |
| ATOM | 2851 | CE | LYS | L | 149 | −27.170 | −46.652 | 33.585 | 1.00 | 62.49 | C |
| ATOM | 2852 | NZ | LYS | L | 149 | −26.922 | −46.308 | 32.153 | 1.00 | 63.39 | N |
| ATOM | 2853 | C | LYS | L | 149 | −28.613 | −40.979 | 36.016 | 1.00 | 58.36 | C |
| ATOM | 2854 | O | LYS | L | 149 | −28.242 | −40.916 | 37.192 | 1.00 | 58.33 | O |
| ATOM | 2855 | N | VAL | L | 150 | −29.678 | −40.333 | 35.547 | 1.00 | 58.85 | N |
| ATOM | 2856 | CA | VAL | L | 150 | −30.527 | −39.491 | 36.396 | 1.00 | 59.50 | C |
| ATOM | 2857 | CB | VAL | L | 150 | −30.469 | −38.011 | 35.965 | 1.00 | 59.68 | C |
| ATOM | 2858 | CG1 | VAL | L | 150 | −31.580 | −37.193 | 36.628 | 1.00 | 59.92 | C |
| ATOM | 2859 | CG2 | VAL | L | 150 | −29.110 | −37.426 | 36.304 | 1.00 | 59.95 | C |
| ATOM | 2860 | C | VAL | L | 150 | −31.961 | −40.021 | 36.382 | 1.00 | 59.79 | C |
| ATOM | 2861 | O | VAL | L | 150 | −32.669 | −39.911 | 35.374 | 1.00 | 59.92 | O |
| ATOM | 2862 | N | ASP | L | 151 | −32.373 | −40.594 | 37.514 | 1.00 | 60.04 | N |
| ATOM | 2863 | CA | ASP | L | 151 | −33.592 | −41.407 | 37.605 | 1.00 | 60.15 | C |
| ATOM | 2864 | CB | ASP | L | 151 | −34.862 | −40.540 | 37.497 | 1.00 | 60.07 | C |
| ATOM | 2865 | CG | ASP | L | 151 | −35.173 | −39.797 | 38.789 | 1.00 | 59.20 | C |
| ATOM | 2866 | OD1 | ASP | L | 151 | −34.762 | −40.285 | 39.865 | 1.00 | 57.76 | O |
| ATOM | 2867 | OD2 | ASP | L | 151 | −35.833 | −38.733 | 38.735 | 1.00 | 59.16 | O |
| ATOM | 2868 | C | ASP | L | 151 | −33.562 | −42.528 | 36.564 | 1.00 | 60.51 | C |
| ATOM | 2869 | O | ASP | L | 151 | −34.546 | −42.773 | 35.861 | 1.00 | 60.78 | O |
| ATOM | 2870 | N | ASN | L | 152 | −32.404 | −43.188 | 36.478 | 1.00 | 60.72 | N |
| ATOM | 2871 | CA | ASN | L | 152 | −32.118 | −44.255 | 35.503 | 1.00 | 60.96 | C |
| ATOM | 2872 | CB | ASN | L | 152 | −32.971 | −45.512 | 35.780 | 1.00 | 61.23 | C |
| ATOM | 2873 | CG | ASN | L | 152 | −32.261 | −46.810 | 35.381 | 1.00 | 62.49 | C |
| ATOM | 2874 | OD1 | ASN | L | 152 | −31.035 | −46.936 | 35.506 | 1.00 | 62.50 | O |
| ATOM | 2875 | ND2 | ASN | L | 152 | −33.036 | −47.783 | 34.897 | 1.00 | 63.20 | N |
| ATOM | 2876 | C | ASN | L | 152 | −32.180 | −43.825 | 34.018 | 1.00 | 60.61 | C |
| ATOM | 2877 | O | ASN | L | 152 | −32.136 | −44.668 | 33.119 | 1.00 | 60.73 | O |
| ATOM | 2878 | N | ALA | L | 153 | −32.267 | −42.516 | 33.781 | 1.00 | 60.19 | N |
| ATOM | 2879 | CA | ALA | L | 153 | −32.176 | −41.932 | 32.439 | 1.00 | 59.86 | C |
| ATOM | 2880 | CB | ALA | L | 153 | −33.146 | −40.772 | 32.313 | 1.00 | 59.89 | C |
| ATOM | 2881 | C | ALA | L | 153 | −30.732 | −41.474 | 32.123 | 1.00 | 59.66 | C |
| ATOM | 2882 | O | ALA | L | 153 | −30.106 | −40.756 | 32.913 | 1.00 | 59.61 | O |
| ATOM | 2883 | N | LEU | L | 154 | −30.215 | −41.896 | 30.971 | 1.00 | 59.12 | N |
| ATOM | 2884 | CA | LEU | L | 154 | −28.793 | −41.711 | 30.637 | 1.00 | 58.59 | C |
| ATOM | 2885 | CB | LEU | L | 154 | −28.284 | −42.864 | 29.747 | 1.00 | 58.75 | C |
| ATOM | 2886 | CG | LEU | L | 154 | −26.997 | −42.755 | 28.909 | 1.00 | 59.16 | C |
| ATOM | 2887 | CD1 | LEU | L | 154 | −25.718 | −42.549 | 29.748 | 1.00 | 58.87 | C |
| ATOM | 2888 | CD2 | LEU | L | 154 | −26.863 | −43.970 | 28.001 | 1.00 | 58.46 | C |
| ATOM | 2889 | C | LEU | L | 154 | −28.476 | −40.338 | 30.040 | 1.00 | 57.96 | C |
| ATOM | 2890 | O | LEU | L | 154 | −28.968 | −39.977 | 28.976 | 1.00 | 57.82 | O |
| ATOM | 2891 | N | GLN | L | 155 | −27.639 | −39.596 | 30.760 | 1.00 | 57.38 | N |
| ATOM | 2892 | CA | GLN | L | 155 | −27.222 | −38.245 | 30.408 | 1.00 | 56.87 | C |
| ATOM | 2893 | CB | GLN | L | 155 | −26.614 | −37.561 | 31.635 | 1.00 | 56.94 | C |
| ATOM | 2894 | CG | GLN | L | 155 | −27.519 | −37.507 | 32.864 | 1.00 | 57.02 | C |
| ATOM | 2895 | CD | GLN | L | 155 | −28.803 | −36.747 | 32.619 | 1.00 | 56.16 | C |
| ATOM | 2896 | OE1 | GLN | L | 155 | −29.772 | −37.287 | 32.074 | 1.00 | 56.65 | O |
| ATOM | 2897 | NE2 | GLN | L | 155 | −28.827 | −35.494 | 33.038 | 1.00 | 56.06 | N |
| ATOM | 2898 | C | GLN | L | 155 | −26.190 | −38.209 | 29.285 | 1.00 | 56.72 | C |
| ATOM | 2899 | O | GLN | L | 155 | −25.396 | −39.145 | 29.126 | 1.00 | 56.36 | O |
| ATOM | 2900 | N | SER | L | 156 | −26.201 | −37.110 | 28.527 | 1.00 | 56.21 | N |
| ATOM | 2901 | CA | SER | L | 156 | −25.199 | −36.869 | 27.487 | 1.00 | 55.80 | C |
| ATOM | 2902 | CB | SER | L | 156 | −25.595 | −37.533 | 26.161 | 1.00 | 55.70 | C |
| ATOM | 2903 | OG | SER | L | 156 | −24.476 | −37.616 | 25.290 | 1.00 | 55.51 | O |
| ATOM | 2904 | C | SER | L | 156 | −24.889 | −35.376 | 27.282 | 1.00 | 55.34 | C |
| ATOM | 2905 | O | SER | L | 156 | −25.799 | −34.539 | 27.250 | 1.00 | 55.05 | O |
| ATOM | 2906 | N | GLY | L | 157 | −23.592 | −35.072 | 27.160 | 1.00 | 54.86 | N |
| ATOM | 2907 | CA | GLY | L | 157 | −23.094 | −33.713 | 26.889 | 1.00 | 54.52 | C |
| ATOM | 2908 | C | GLY | L | 157 | −23.150 | −32.784 | 28.085 | 1.00 | 53.91 | C |
| ATOM | 2909 | O | GLY | L | 157 | −22.840 | −31.599 | 27.995 | 1.00 | 53.97 | O |
| ATOM | 2910 | N | ASN | L | 158 | −23.513 | −33.365 | 29.217 | 1.00 | 53.52 | N |
| ATOM | 2911 | CA | ASN | L | 158 | −23.877 | −32.659 | 30.418 | 1.00 | 53.07 | C |
| ATOM | 2912 | CB | ASN | L | 158 | −25.079 | −33.395 | 31.002 | 1.00 | 53.77 | C |
| ATOM | 2913 | CG | ASN | L | 158 | −26.108 | −32.466 | 31.565 | 1.00 | 55.25 | C |
| ATOM | 2914 | OD1 | ASN | L | 158 | −26.009 | −31.249 | 31.410 | 1.00 | 58.83 | O |
| ATOM | 2915 | ND2 | ASN | L | 158 | −27.112 | −33.027 | 32.225 | 1.00 | 54.83 | N |
| ATOM | 2916 | C | ASN | L | 158 | −22.747 | −32.638 | 31.453 | 1.00 | 52.36 | C |
| ATOM | 2917 | O | ASN | L | 158 | −22.838 | −31.953 | 32.476 | 1.00 | 51.98 | O |
| ATOM | 2918 | N | SER | L | 159 | −21.695 | −33.414 | 31.196 | 1.00 | 51.47 | N |
| ATOM | 2919 | CA | SER | L | 159 | −20.602 | −33.565 | 32.158 | 1.00 | 50.68 | C |
| ATOM | 2920 | CB | SER | L | 159 | −20.656 | −34.946 | 32.808 | 1.00 | 50.80 | C |
| ATOM | 2921 | OG | SER | L | 159 | −20.723 | −35.966 | 31.833 | 1.00 | 49.28 | O |
| ATOM | 2922 | C | SER | L | 159 | −19.238 | −33.356 | 31.529 | 1.00 | 50.44 | C |
| ATOM | 2923 | O | SER | L | 159 | −19.066 | −33.590 | 30.341 | 1.00 | 50.37 | O |
| ATOM | 2924 | N | GLN | L | 160 | −18.278 | −32.912 | 32.340 | 1.00 | 50.02 | N |
| ATOM | 2925 | CA | GLN | L | 160 | −16.882 | −32.779 | 31.923 | 1.00 | 49.44 | C |
| ATOM | 2926 | CB | GLN | L | 160 | −16.350 | −31.359 | 32.183 | 1.00 | 49.61 | C |
| ATOM | 2927 | CG | GLN | L | 160 | −17.269 | −30.238 | 31.705 | 1.00 | 51.10 | C |
| ATOM | 2928 | CD | GLN | L | 160 | −16.525 | −28.968 | 31.342 | 1.00 | 52.58 | C |
| ATOM | 2929 | OE1 | GLN | L | 160 | −16.807 | −27.894 | 31.878 | 1.00 | 52.68 | O |
| ATOM | 2930 | NE2 | GLN | L | 160 | −15.570 | −29.083 | 30.423 | 1.00 | 53.76 | N |
| ATOM | 2931 | C | GLN | L | 160 | −16.010 | −33.791 | 32.666 | 1.00 | 48.74 | C |
| ATOM | 2932 | O | GLN | L | 160 | −16.197 | −34.028 | 33.867 | 1.00 | 48.37 | O |
| ATOM | 2933 | N | GLU | L | 161 | −15.061 | −34.383 | 31.948 | 1.00 | 47.31 | N |
| ATOM | 2934 | CA | GLU | L | 161 | −14.074 | −35.230 | 32.575 | 1.00 | 46.28 | C |
| ATOM | 2935 | CB | GLU | L | 161 | −13.892 | −36.544 | 31.820 | 1.00 | 46.11 | C |
| ATOM | 2936 | CG | GLU | L | 161 | −15.139 | −37.400 | 31.798 | 1.00 | 46.95 | C |
| ATOM | 2937 | CD | GLU | L | 161 | −14.899 | −38.771 | 31.227 | 1.00 | 47.86 | C |
| ATOM | 2938 | OE1 | GLU | L | 161 | −15.763 | −39.638 | 31.434 | 1.00 | 48.66 | O |
| ATOM | 2939 | OE2 | GLU | L | 161 | −13.849 | −38.995 | 30.581 | 1.00 | 49.17 | O |
| ATOM | 2940 | C | GLU | L | 161 | −12.749 | −34.514 | 32.704 | 1.00 | 45.66 | C |
| ATOM | 2941 | O | GLU | L | 161 | −12.394 | −33.659 | 31.884 | 1.00 | 45.72 | O |
| ATOM | 2942 | N | SER | L | 162 | −12.033 | −34.863 | 33.763 | 1.00 | 44.46 | N |
| ATOM | 2943 | CA | SER | L | 162 | −10.680 | −34.412 | 33.979 | 1.00 | 43.75 | C |
| ATOM | 2944 | CB | SER | L | 162 | −10.671 | −33.298 | 35.016 | 1.00 | 43.60 | C |
| ATOM | 2945 | OG | SER | L | 162 | −9.427 | −32.637 | 35.027 | 1.00 | 44.93 | O |
| ATOM | 2946 | C | SER | L | 162 | −9.871 | −35.624 | 34.446 | 1.00 | 43.09 | C |
| ATOM | 2947 | O | SER | L | 162 | −10.400 | −36.489 | 35.143 | 1.00 | 42.27 | O |
| ATOM | 2948 | N | VAL | L | 163 | −8.604 | −35.696 | 34.034 | 1.00 | 42.63 | N |
| ATOM | 2949 | CA | VAL | L | 163 | −7.749 | −36.845 | 34.330 | 1.00 | 42.03 | C |
| ATOM | 2950 | CB | VAL | L | 163 | −7.536 | −37.732 | 33.088 | 1.00 | 42.13 | C |
| ATOM | 2951 | CG1 | VAL | L | 163 | −6.996 | −39.095 | 33.487 | 1.00 | 40.39 | C |
| ATOM | 2952 | CG2 | VAL | L | 163 | −8.834 | −37.891 | 32.303 | 1.00 | 44.07 | C |
| ATOM | 2953 | C | VAL | L | 163 | −6.379 | −36.365 | 34.774 | 1.00 | 42.03 | C |
| ATOM | 2954 | O | VAL | L | 163 | −5.824 | −35.430 | 34.187 | 1.00 | 41.85 | O |
| ATOM | 2955 | N | THR | L | 164 | −5.816 | −37.016 | 35.789 | 1.00 | 41.04 | N |
| ATOM | 2956 | CA | THR | L | 164 | −4.472 | −36.675 | 36.243 | 1.00 | 40.90 | C |
| ATOM | 2957 | CB | THR | L | 164 | −4.191 | −37.269 | 37.635 | 1.00 | 40.75 | C |
| ATOM | 2958 | OG1 | THR | L | 164 | −4.437 | −38.680 | 37.591 | 1.00 | 38.84 | O |
| ATOM | 2959 | CG2 | THR | L | 164 | −5.077 | −36.592 | 38.709 | 1.00 | 40.80 | C |
| ATOM | 2960 | C | THR | L | 164 | −3.426 | −37.222 | 35.282 | 1.00 | 40.80 | C |
| ATOM | 2961 | O | THR | L | 164 | −3.712 | −38.084 | 34.467 | 1.00 | 40.83 | O |
| ATOM | 2962 | N | GLU | L | 165 | −2.205 | −36.722 | 35.390 | 1.00 | 41.42 | N |
| ATOM | 2963 | CA | GLU | L | 165 | −1.074 | −37.349 | 34.726 | 1.00 | 41.92 | C |
| ATOM | 2964 | CB | GLU | L | 165 | 0.162 | −36.465 | 34.860 | 1.00 | 42.62 | C |
| ATOM | 2965 | CG | GLU | L | 165 | 0.124 | −35.226 | 33.962 | 1.00 | 46.06 | C |
| ATOM | 2966 | CD | GLU | L | 165 | 0.032 | −35.597 | 32.486 | 1.00 | 50.50 | C |
| ATOM | 2967 | OE1 | GLU | L | 165 | 1.092 | −35.631 | 31.820 | 1.00 | 53.24 | O |
| ATOM | 2968 | OE2 | GLU | L | 165 | −1.091 | −35.881 | 32.005 | 1.00 | 52.11 | O |
| ATOM | 2969 | C | GLU | L | 165 | −0.837 | −38.705 | 35.388 | 1.00 | 41.57 | C |
| ATOM | 2970 | O | GLU | L | 165 | −1.318 | −38.943 | 36.498 | 1.00 | 40.89 | O |
| ATOM | 2971 | N | GLN | L | 166 | −0.117 | −39.595 | 34.719 | 1.00 | 40.87 | N |
| ATOM | 2972 | CA | GLN | L | 166 | 0.176 | −40.875 | 35.338 | 1.00 | 41.14 | C |
| ATOM | 2973 | CB | GLN | L | 166 | 0.974 | −41.787 | 34.412 | 1.00 | 40.70 | C |
| ATOM | 2974 | CG | GLN | L | 166 | 0.942 | −43.226 | 34.879 | 1.00 | 40.67 | C |
| ATOM | 2975 | CD | GLN | L | 166 | 1.673 | −44.165 | 33.958 | 1.00 | 39.62 | C |
| ATOM | 2976 | OE1 | GLN | L | 166 | 2.696 | −43.809 | 33.365 | 1.00 | 38.09 | O |
| ATOM | 2977 | NE2 | GLN | L | 166 | 1.158 | −45.395 | 33.842 | 1.00 | 38.46 | N |
| ATOM | 2978 | C | GLN | L | 166 | 0.921 | −40.656 | 36.647 | 1.00 | 40.97 | C |
| ATOM | 2979 | O | GLN | L | 166 | 1.928 | −39.931 | 36.696 | 1.00 | 40.26 | O |
| ATOM | 2980 | N | ASP | L | 167 | 0.397 | −41.252 | 37.711 | 1.00 | 41.69 | N |
| ATOM | 2981 | CA | ASP | L | 167 | 1.050 | −41.184 | 39.022 | 1.00 | 42.90 | C |
| ATOM | 2982 | CB | ASP | L | 167 | 0.177 | −41.801 | 40.117 | 1.00 | 42.61 | C |
| ATOM | 2983 | CG | ASP | L | 167 | 0.648 | −41.431 | 41.504 | 1.00 | 43.25 | C |
| ATOM | 2984 | OD1 | ASP | L | 167 | 1.873 | −41.371 | 41.750 | 1.00 | 42.73 | O |
| ATOM | 2985 | OD2 | ASP | L | 167 | −0.220 | −41.203 | 42.362 | 1.00 | 46.93 | O |
| ATOM | 2986 | C | ASP | L | 167 | 2.374 | −41.913 | 38.917 | 1.00 | 43.53 | C |
| ATOM | 2987 | O | ASP | L | 167 | 2.400 | −43.094 | 38.609 | 1.00 | 43.95 | O |
| ATOM | 2988 | N | SER | L | 168 | 3.463 | −41.193 | 39.160 | 1.00 | 44.81 | N |
| ATOM | 2989 | CA | SER | L | 168 | 4.806 | −41.696 | 38.925 | 1.00 | 46.54 | C |
| ATOM | 2990 | CB | SER | L | 168 | 5.816 | −40.569 | 39.084 | 1.00 | 46.84 | C |
| ATOM | 2991 | OG | SER | L | 168 | 5.887 | −40.176 | 40.444 | 1.00 | 48.68 | O |
| ATOM | 2992 | C | SER | L | 168 | 5.211 | −42.831 | 39.856 | 1.00 | 47.55 | C |
| ATOM | 2993 | O | SER | L | 168 | 6.193 | −43.529 | 39.580 | 1.00 | 48.20 | O |
| ATOM | 2994 | N | LYS | L | 169 | 4.465 | −43.020 | 40.945 | 1.00 | 47.97 | N |
| ATOM | 2995 | CA | LYS | L | 169 | 4.854 | −43.969 | 41.974 | 1.00 | 48.43 | C |
| ATOM | 2996 | CB | LYS | L | 169 | 4.656 | −43.362 | 43.365 | 1.00 | 49.07 | C |
| ATOM | 2997 | CG | LYS | L | 169 | 5.649 | −42.242 | 43.717 | 1.00 | 50.13 | C |
| ATOM | 2998 | CD | LYS | L | 169 | 5.154 | −41.420 | 44.908 | 1.00 | 52.63 | C |
| ATOM | 2999 | CE | LYS | L | 169 | 3.748 | −40.841 | 44.628 | 1.00 | 54.12 | C |
| ATOM | 3000 | NZ | LYS | L | 169 | 3.022 | −40.433 | 45.871 | 1.00 | 54.26 | N |
| ATOM | 3001 | C | LYS | L | 169 | 4.129 | −45.304 | 41.854 | 1.00 | 48.20 | C |
| ATOM | 3002 | O | LYS | L | 169 | 4.754 | −46.368 | 42.023 | 1.00 | 48.55 | O |
| ATOM | 3003 | N | ASP | L | 170 | 2.825 | −45.249 | 41.565 | 1.00 | 47.03 | N |
| ATOM | 3004 | CA | ASP | L | 170 | 2.034 | −46.464 | 41.353 | 1.00 | 45.87 | C |
| ATOM | 3005 | CB | ASP | L | 170 | 0.892 | −46.573 | 42.378 | 1.00 | 46.41 | C |
| ATOM | 3006 | CG | ASP | L | 170 | −0.148 | −45.474 | 42.248 | 1.00 | 48.66 | C |
| ATOM | 3007 | OD1 | ASP | L | 170 | −0.320 | −44.909 | 41.140 | 1.00 | 50.59 | O |
| ATOM | 3008 | OD2 | ASP | L | 170 | −0.822 | −45.189 | 43.270 | 1.00 | 48.97 | O |
| ATOM | 3009 | C | ASP | L | 170 | 1.529 | −46.691 | 39.913 | 1.00 | 44.66 | C |
| ATOM | 3010 | O | ASP | L | 170 | 0.795 | −47.655 | 39.652 | 1.00 | 43.93 | O |
| ATOM | 3011 | N | SER | L | 171 | 1.928 | −45.805 | 38.992 | 1.00 | 43.20 | N |
| ATOM | 3012 | CA | SER | L | 171 | 1.581 | −45.921 | 37.566 | 1.00 | 41.93 | C |
| ATOM | 3013 | CB | SER | L | 171 | 2.172 | −47.207 | 36.963 | 1.00 | 41.64 | C |
| ATOM | 3014 | OG | SER | L | 171 | 3.579 | −47.195 | 37.022 | 1.00 | 42.01 | O |
| ATOM | 3015 | C | SER | L | 171 | 0.084 | −45.873 | 37.275 | 1.00 | 41.12 | C |
| ATOM | 3016 | O | SER | L | 171 | −0.359 | −46.376 | 36.241 | 1.00 | 40.36 | O |
| ATOM | 3017 | N | THR | L | 172 | −0.692 | −45.278 | 38.178 | 1.00 | 40.59 | N |
| ATOM | 3018 | CA | THR | L | 172 | −2.141 | −45.201 | 37.998 | 1.00 | 40.44 | C |
| ATOM | 3019 | CB | THR | L | 172 | −2.930 | −45.645 | 39.254 | 1.00 | 40.51 | C |
| ATOM | 3020 | OG1 | THR | L | 172 | −2.617 | −44.774 | 40.358 | 1.00 | 39.84 | O |
| ATOM | 3021 | CG2 | THR | L | 172 | −2.616 | −47.107 | 39.619 | 1.00 | 40.52 | C |
| ATOM | 3022 | C | THR | L | 172 | −2.611 | −43.801 | 37.629 | 1.00 | 40.46 | C |
| ATOM | 3023 | O | THR | L | 172 | −1.871 | −42.828 | 37.750 | 1.00 | 40.36 | O |
| ATOM | 3024 | N | TYR | L | 173 | −3.862 | −43.737 | 37.191 | 1.00 | 40.72 | N |
| ATOM | 3025 | CA | TYR | L | 173 | −4.541 | −42.508 | 36.864 | 1.00 | 41.46 | C |
| ATOM | 3026 | CB | TYR | L | 173 | −5.080 | −42.577 | 35.433 | 1.00 | 41.57 | C |
| ATOM | 3027 | CG | TYR | L | 173 | −4.008 | −42.620 | 34.370 | 1.00 | 42.50 | C |
| ATOM | 3028 | CD1 | TYR | L | 173 | −3.623 | −43.831 | 33.796 | 1.00 | 43.91 | C |
| ATOM | 3029 | CE1 | TYR | L | 173 | −2.651 | −43.876 | 32.804 | 1.00 | 45.82 | C |
| ATOM | 3030 | CZ | TYR | L | 173 | −2.044 | −42.699 | 32.393 | 1.00 | 46.00 | C |
| ATOM | 3031 | OH | TYR | L | 173 | −1.079 | −42.735 | 31.418 | 1.00 | 47.44 | O |
| ATOM | 3032 | CE2 | TYR | L | 173 | −2.402 | −41.485 | 32.955 | 1.00 | 44.35 | C |
| ATOM | 3033 | CD2 | TYR | L | 173 | −3.384 | −41.456 | 33.942 | 1.00 | 42.64 | C |
| ATOM | 3034 | C | TYR | L | 173 | −5.720 | −42.354 | 37.781 | 1.00 | 41.61 | C |
| ATOM | 3035 | O | TYR | L | 173 | −6.248 | −43.342 | 38.280 | 1.00 | 41.72 | O |
| ATOM | 3036 | N | SER | L | 174 | −6.131 | −41.114 | 37.996 | 1.00 | 41.79 | N |
| ATOM | 3037 | CA | SER | L | 174 | −7.415 | −40.837 | 38.604 | 1.00 | 42.38 | C |
| ATOM | 3038 | CB | SER | L | 174 | −7.230 | −40.290 | 40.015 | 1.00 | 42.54 | C |
| ATOM | 3039 | OG | SER | L | 174 | −6.603 | −41.274 | 40.829 | 1.00 | 43.13 | O |
| ATOM | 3040 | C | SER | L | 174 | −8.198 | −39.875 | 37.723 | 1.00 | 43.05 | C |
| ATOM | 3041 | O | SER | L | 174 | −7.618 | −39.113 | 36.956 | 1.00 | 42.71 | O |
| ATOM | 3042 | N | LEU | L | 175 | −9.519 | −39.925 | 37.830 | 1.00 | 43.91 | N |
| ATOM | 3043 | CA | LEU | L | 175 | −10.395 | −39.204 | 36.926 | 1.00 | 44.43 | C |
| ATOM | 3044 | CB | LEU | L | 175 | −10.908 | −40.142 | 35.826 | 1.00 | 44.56 | C |
| ATOM | 3045 | CG | LEU | L | 175 | −11.802 | −39.610 | 34.699 | 1.00 | 44.08 | C |
| ATOM | 3046 | CD1 | LEU | L | 175 | −11.566 | −40.421 | 33.431 | 1.00 | 44.07 | C |
| ATOM | 3047 | CD2 | LEU | L | 175 | −13.278 | −39.637 | 35.076 | 1.00 | 44.15 | C |
| ATOM | 3048 | C | LEU | L | 175 | −11.575 | −38.645 | 37.685 | 1.00 | 44.98 | C |
| ATOM | 3049 | O | LEU | L | 175 | −12.163 | −39.334 | 38.526 | 1.00 | 44.98 | O |
| ATOM | 3050 | N | SER | L | 176 | −11.930 | −37.400 | 37.369 | 1.00 | 45.15 | N |
| ATOM | 3051 | CA | SER | L | 176 | −13.175 | −36.821 | 37.849 | 1.00 | 45.43 | C |
| ATOM | 3052 | CB | SER | L | 176 | −12.928 | −35.498 | 38.575 | 1.00 | 45.42 | C |
| ATOM | 3053 | OG | SER | L | 176 | −12.511 | −34.498 | 37.666 | 1.00 | 46.05 | O |
| ATOM | 3054 | C | SER | L | 176 | −14.154 | −36.605 | 36.704 | 1.00 | 45.61 | C |
| ATOM | 3055 | O | SER | L | 176 | −13.777 | −36.183 | 35.603 | 1.00 | 45.68 | O |
| ATOM | 3056 | N | SER | L | 177 | −15.412 | −36.910 | 36.972 | 1.00 | 45.58 | N |
| ATOM | 3057 | CA | SER | L | 177 | −16.491 | −36.550 | 36.072 | 1.00 | 45.67 | C |
| ATOM | 3058 | CB | SER | L | 177 | −17.203 | −37.780 | 35.537 | 1.00 | 45.17 | C |
| ATOM | 3059 | OG | SER | L | 177 | −18.321 | −37.378 | 34.782 | 1.00 | 45.68 | O |
| ATOM | 3060 | C | SER | L | 177 | −17.455 | −35.678 | 36.839 | 1.00 | 45.79 | C |
| ATOM | 3061 | O | SER | L | 177 | −18.055 | −36.117 | 37.831 | 1.00 | 45.89 | O |
| ATOM | 3062 | N | THR | L | 178 | −17.581 | −34.437 | 36.390 | 1.00 | 45.89 | N |
| ATOM | 3063 | CA | THR | L | 178 | −18.428 | −33.459 | 37.047 | 1.00 | 46.45 | C |
| ATOM | 3064 | CB | THR | L | 178 | −17.673 | −32.133 | 37.287 | 1.00 | 46.06 | C |
| ATOM | 3065 | OG1 | THR | L | 178 | −16.401 | −32.406 | 37.897 | 1.00 | 46.52 | O |
| ATOM | 3066 | CG2 | THR | L | 178 | −18.474 | −31.210 | 38.177 | 1.00 | 45.41 | C |
| ATOM | 3067 | C | THR | L | 178 | −19.670 | −33.229 | 36.195 | 1.00 | 47.04 | C |
| ATOM | 3068 | O | THR | L | 178 | −19.586 | −32.718 | 35.079 | 1.00 | 47.22 | O |
| ATOM | 3069 | N | LEU | L | 179 | −20.816 | −33.654 | 36.711 | 1.00 | 47.78 | N |
| ATOM | 3070 | CA | LEU | L | 179 | −22.093 | −33.364 | 36.082 | 1.00 | 48.51 | C |
| ATOM | 3071 | CB | LEU | L | 179 | −23.084 | −34.472 | 36.397 | 1.00 | 48.38 | C |
| ATOM | 3072 | CG | LEU | L | 179 | −24.524 | −34.395 | 35.897 | 1.00 | 47.38 | C |
| ATOM | 3073 | CD1 | LEU | L | 179 | −24.623 | −34.819 | 34.448 | 1.00 | 48.80 | C |
| ATOM | 3074 | CD2 | LEU | L | 179 | −25.365 | −35.305 | 36.743 | 1.00 | 46.69 | C |
| ATOM | 3075 | C | LEU | L | 179 | −22.612 | −32.020 | 36.591 | 1.00 | 49.62 | C |
| ATOM | 3076 | O | LEU | L | 179 | −22.657 | −31.784 | 37.798 | 1.00 | 50.02 | O |
| ATOM | 3077 | N | THR | L | 180 | −22.996 | −31.146 | 35.667 | 1.00 | 50.71 | N |
| ATOM | 3078 | CA | THR | L | 180 | −23.496 | −29.815 | 36.006 | 1.00 | 52.07 | C |
| ATOM | 3079 | CB | THR | L | 180 | −22.694 | −28.724 | 35.270 | 1.00 | 51.82 | C |
| ATOM | 3080 | OG1 | THR | L | 180 | −21.322 | −28.800 | 35.665 | 1.00 | 51.77 | O |
| ATOM | 3081 | CG2 | THR | L | 180 | −23.226 | −27.335 | 35.589 | 1.00 | 52.12 | C |
| ATOM | 3082 | C | THR | L | 180 | −24.989 | −29.669 | 35.675 | 1.00 | 53.27 | C |
| ATOM | 3083 | O | THR | L | 180 | −25.410 | −29.931 | 34.548 | 1.00 | 53.34 | O |
| ATOM | 3084 | N | LEU | L | 181 | −25.779 | −29.263 | 36.669 | 1.00 | 54.55 | N |
| ATOM | 3085 | CA | LEU | L | 181 | −27.216 | −29.052 | 36.490 | 1.00 | 56.14 | C |
| ATOM | 3086 | CB | LEU | L | 181 | −28.014 | −30.177 | 37.155 | 1.00 | 55.65 | C |
| ATOM | 3087 | CG | LEU | L | 181 | −28.445 | −31.389 | 36.330 | 1.00 | 55.74 | C |
| ATOM | 3088 | CD1 | LEU | L | 181 | −27.310 | −32.338 | 36.119 | 1.00 | 55.89 | C |
| ATOM | 3089 | CD2 | LEU | L | 181 | −29.576 | −32.124 | 37.022 | 1.00 | 55.96 | C |
| ATOM | 3090 | C | LEU | L | 181 | −27.656 | −27.713 | 37.070 | 1.00 | 57.32 | C |
| ATOM | 3091 | O | LEU | L | 181 | −27.043 | −27.201 | 38.020 | 1.00 | 57.58 | O |
| ATOM | 3092 | N | SER | L | 182 | −28.713 | −27.147 | 36.494 | 1.00 | 58.84 | N |
| ATOM | 3093 | CA | SER | L | 182 | −29.378 | −25.994 | 37.096 | 1.00 | 60.52 | C |
| ATOM | 3094 | CB | SER | L | 182 | −30.420 | −25.394 | 36.146 | 1.00 | 60.40 | C |
| ATOM | 3095 | OG | SER | L | 182 | −31.432 | −26.335 | 35.825 | 1.00 | 60.57 | O |
| ATOM | 3096 | C | SER | L | 182 | −30.046 | −26.463 | 38.378 | 1.00 | 61.50 | C |
| ATOM | 3097 | O | SER | L | 182 | −30.566 | −27.584 | 38.438 | 1.00 | 61.98 | O |
| ATOM | 3098 | N | LYS | L | 183 | −30.007 | −25.614 | 39.404 | 1.00 | 62.87 | N |
| ATOM | 3099 | CA | LYS | L | 183 | −30.683 | −25.880 | 40.682 | 1.00 | 63.88 | C |
| ATOM | 3100 | CB | LYS | L | 183 | −30.743 | −24.596 | 41.520 | 1.00 | 64.08 | C |
| ATOM | 3101 | CG | LYS | L | 183 | −31.223 | −24.769 | 42.964 | 1.00 | 64.72 | C |
| ATOM | 3102 | CD | LYS | L | 183 | −31.726 | −23.451 | 43.518 | 1.00 | 65.50 | C |
| ATOM | 3103 | CE | LYS | L | 183 | −32.359 | −23.625 | 44.887 | 1.00 | 65.21 | C |
| ATOM | 3104 | NZ | LYS | L | 183 | −31.395 | −23.342 | 45.977 | 1.00 | 65.52 | N |
| ATOM | 3105 | C | LYS | L | 183 | −32.097 | −26.426 | 40.457 | 1.00 | 64.32 | C |
| ATOM | 3106 | O | LYS | L | 183 | −32.582 | −27.254 | 41.230 | 1.00 | 64.35 | O |
| ATOM | 3107 | N | ALA | L | 184 | −32.737 | −25.951 | 39.388 | 1.00 | 65.10 | N |
| ATOM | 3108 | CA | ALA | L | 184 | −34.093 | −26.346 | 39.028 | 1.00 | 65.83 | C |
| ATOM | 3109 | CB | ALA | L | 184 | −34.634 | −25.446 | 37.930 | 1.00 | 65.84 | C |
| ATOM | 3110 | C | ALA | L | 184 | −34.136 | −27.795 | 38.588 | 1.00 | 66.44 | C |
| ATOM | 3111 | O | ALA | L | 184 | −35.001 | −28.551 | 39.027 | 1.00 | 66.48 | O |
| ATOM | 3112 | N | ASP | L | 185 | −33.197 | −28.183 | 37.727 | 1.00 | 67.10 | N |
| ATOM | 3113 | CA | ASP | L | 185 | −33.135 | −29.561 | 37.243 | 1.00 | 67.84 | C |
| ATOM | 3114 | CB | ASP | L | 185 | −32.239 | −29.663 | 36.008 | 1.00 | 67.94 | C |
| ATOM | 3115 | CG | ASP | L | 185 | −32.941 | −29.198 | 34.753 | 1.00 | 68.17 | C |
| ATOM | 3116 | OD1 | ASP | L | 185 | −34.185 | −29.316 | 34.696 | 1.00 | 68.93 | O |
| ATOM | 3117 | OD2 | ASP | L | 185 | −32.259 | −28.716 | 33.828 | 1.00 | 67.82 | O |
| ATOM | 3118 | C | ASP | L | 185 | −32.708 | −30.554 | 38.321 | 1.00 | 68.13 | C |
| ATOM | 3119 | O | ASP | L | 185 | −33.208 | −31.681 | 38.366 | 1.00 | 67.93 | O |
| ATOM | 3120 | N | TYR | L | 186 | −31.796 | −30.130 | 39.190 | 1.00 | 68.69 | N |
| ATOM | 3121 | CA | TYR | L | 186 | −31.350 | −30.983 | 40.286 | 1.00 | 69.46 | C |
| ATOM | 3122 | CB | TYR | L | 186 | −30.166 | −30.360 | 41.042 | 1.00 | 69.41 | C |
| ATOM | 3123 | CG | TYR | L | 186 | −29.631 | −31.222 | 42.176 | 1.00 | 69.31 | C |
| ATOM | 3124 | CD1 | TYR | L | 186 | −28.923 | −32.402 | 41.914 | 1.00 | 69.10 | C |
| ATOM | 3125 | CE1 | TYR | L | 186 | −28.428 | −33.201 | 42.952 | 1.00 | 68.56 | C |
| ATOM | 3126 | CZ | TYR | L | 186 | −28.638 | −32.818 | 44.266 | 1.00 | 68.54 | C |
| ATOM | 3127 | OH | TYR | L | 186 | −28.152 | −33.601 | 45.287 | 1.00 | 68.37 | O |
| ATOM | 3128 | CE2 | TYR | L | 186 | −29.330 | −31.646 | 44.558 | 1.00 | 68.78 | C |
| ATOM | 3129 | CD2 | TYR | L | 186 | −29.823 | −30.856 | 43.511 | 1.00 | 69.13 | C |
| ATOM | 3130 | C | TYR | L | 186 | −32.493 | −31.303 | 41.243 | 1.00 | 69.98 | C |
| ATOM | 3131 | O | TYR | L | 186 | −32.678 | −32.462 | 41.617 | 1.00 | 70.11 | O |
| ATOM | 3132 | N | GLU | L | 187 | −33.261 | −30.272 | 41.605 | 1.00 | 70.56 | N |
| ATOM | 3133 | CA | GLU | L | 187 | −34.327 | −30.371 | 42.608 | 1.00 | 71.22 | C |
| ATOM | 3134 | CB | GLU | L | 187 | −34.707 | −28.977 | 43.127 | 1.00 | 71.29 | C |
| ATOM | 3135 | CG | GLU | L | 187 | −33.827 | −28.467 | 44.252 | 1.00 | 72.34 | C |
| ATOM | 3136 | CD | GLU | L | 187 | −34.144 | −27.033 | 44.652 | 1.00 | 74.43 | C |
| ATOM | 3137 | OE1 | GLU | L | 187 | −33.683 | −26.609 | 45.734 | 1.00 | 75.80 | O |
| ATOM | 3138 | OE2 | GLU | L | 187 | −34.846 | −26.321 | 43.894 | 1.00 | 75.41 | O |
| ATOM | 3139 | C | GLU | L | 187 | −35.594 | −31.104 | 42.159 | 1.00 | 71.32 | C |
| ATOM | 3140 | O | GLU | L | 187 | −36.520 | −31.265 | 42.957 | 1.00 | 71.41 | O |
| ATOM | 3141 | N | LYS | L | 188 | −35.640 | −31.553 | 40.905 | 1.00 | 71.42 | N |
| ATOM | 3142 | CA | LYS | L | 188 | −36.829 | −32.244 | 40.395 | 1.00 | 71.51 | C |
| ATOM | 3143 | CB | LYS | L | 188 | −37.490 | −31.453 | 39.251 | 1.00 | 71.68 | C |
| ATOM | 3144 | CG | LYS | L | 188 | −36.628 | −31.236 | 38.018 | 1.00 | 72.26 | C |
| ATOM | 3145 | CD | LYS | L | 188 | −37.464 | −30.812 | 36.813 | 1.00 | 73.36 | C |
| ATOM | 3146 | CE | LYS | L | 188 | −38.037 | −32.024 | 36.075 | 1.00 | 73.61 | C |
| ATOM | 3147 | NZ | LYS | L | 188 | −38.527 | −31.673 | 34.711 | 1.00 | 74.19 | N |
| ATOM | 3148 | C | LYS | L | 188 | −36.599 | −33.708 | 40.000 | 1.00 | 71.39 | C |
| ATOM | 3149 | O | LYS | L | 188 | −37.452 | −34.320 | 39.351 | 1.00 | 71.65 | O |
| ATOM | 3150 | N | HIS | L | 189 | −35.453 | −34.264 | 40.395 | 1.00 | 71.06 | N |
| ATOM | 3151 | CA | HIS | L | 189 | −35.184 | −35.700 | 40.220 | 1.00 | 70.51 | C |
| ATOM | 3152 | CB | HIS | L | 189 | −34.169 | −35.930 | 39.093 | 1.00 | 70.79 | C |
| ATOM | 3153 | CG | HIS | L | 189 | −34.709 | −35.625 | 37.727 | 1.00 | 71.53 | C |
| ATOM | 3154 | ND1 | HIS | L | 189 | −35.555 | −36.481 | 37.053 | 1.00 | 71.94 | N |
| ATOM | 3155 | CE1 | HIS | L | 189 | −35.872 | −35.953 | 35.883 | 1.00 | 72.00 | C |
| ATOM | 3156 | NE2 | HIS | L | 189 | −35.260 | −34.787 | 35.772 | 1.00 | 71.74 | N |
| ATOM | 3157 | CD2 | HIS | L | 189 | −34.529 | −34.556 | 36.913 | 1.00 | 71.36 | C |
| ATOM | 3158 | C | HIS | L | 189 | −34.731 | −36.343 | 41.533 | 1.00 | 69.79 | C |
| ATOM | 3159 | O | HIS | L | 189 | −34.410 | −35.635 | 42.491 | 1.00 | 69.84 | O |
| ATOM | 3160 | N | LYS | L | 190 | −34.715 | −37.674 | 41.586 | 1.00 | 68.96 | N |
| ATOM | 3161 | CA | LYS | L | 190 | −34.418 | −38.366 | 42.846 | 1.00 | 68.18 | C |
| ATOM | 3162 | CB | LYS | L | 190 | −35.569 | −39.300 | 43.248 | 1.00 | 68.36 | C |
| ATOM | 3163 | CG | LYS | L | 190 | −35.775 | −39.396 | 44.758 | 1.00 | 69.06 | C |
| ATOM | 3164 | CD | LYS | L | 190 | −36.735 | −40.523 | 45.149 | 1.00 | 70.74 | C |
| ATOM | 3165 | CE | LYS | L | 190 | −35.997 | −41.851 | 45.349 | 1.00 | 71.45 | C |
| ATOM | 3166 | NZ | LYS | L | 190 | −36.894 | −42.914 | 45.899 | 1.00 | 71.90 | N |
| ATOM | 3167 | C | LYS | L | 190 | −33.089 | −39.123 | 42.871 | 1.00 | 67.37 | C |
| ATOM | 3168 | O | LYS | L | 190 | −32.261 | −38.888 | 43.748 | 1.00 | 67.23 | O |
| ATOM | 3169 | N | VAL | L | 191 | −32.899 | −40.039 | 41.925 | 1.00 | 66.59 | N |
| ATOM | 3170 | CA | VAL | L | 191 | −31.697 | −40.884 | 41.892 | 1.00 | 65.53 | C |
| ATOM | 3171 | CB | VAL | L | 191 | −32.008 | −42.337 | 41.424 | 1.00 | 65.74 | C |
| ATOM | 3172 | CG1 | VAL | L | 191 | −30.815 | −43.252 | 41.687 | 1.00 | 65.10 | C |
| ATOM | 3173 | CG2 | VAL | L | 191 | −33.257 | −42.887 | 42.125 | 1.00 | 65.65 | C |
| ATOM | 3174 | C | VAL | L | 191 | −30.612 | −40.275 | 41.001 | 1.00 | 64.80 | C |
| ATOM | 3175 | O | VAL | L | 191 | −30.862 | −39.939 | 39.834 | 1.00 | 64.87 | O |
| ATOM | 3176 | N | TYR | L | 192 | −29.418 | −40.129 | 41.574 | 1.00 | 63.42 | N |
| ATOM | 3177 | CA | TYR | L | 192 | −28.230 | −39.703 | 40.844 | 1.00 | 62.21 | C |
| ATOM | 3178 | CB | TYR | L | 192 | −27.709 | −38.365 | 41.385 | 1.00 | 62.28 | C |
| ATOM | 3179 | CG | TYR | L | 192 | −28.593 | −37.195 | 41.031 | 1.00 | 62.25 | C |
| ATOM | 3180 | CD1 | TYR | L | 192 | −29.598 | −36.760 | 41.905 | 1.00 | 62.28 | C |
| ATOM | 3181 | CE1 | TYR | L | 192 | −30.424 | −35.688 | 41.573 | 1.00 | 62.42 | C |
| ATOM | 3182 | CZ | TYR | L | 192 | −30.252 | −35.048 | 40.347 | 1.00 | 63.07 | C |
| ATOM | 3183 | OH | TYR | L | 192 | −31.057 | −33.988 | 39.998 | 1.00 | 62.94 | O |
| ATOM | 3184 | CE2 | TYR | L | 192 | −29.263 | −35.467 | 39.464 | 1.00 | 62.76 | C |
| ATOM | 3185 | CD2 | TYR | L | 192 | −28.444 | −36.536 | 39.810 | 1.00 | 62.20 | C |
| ATOM | 3186 | C | TYR | L | 192 | −27.156 | −40.785 | 40.939 | 1.00 | 61.41 | C |
| ATOM | 3187 | O | TYR | L | 192 | −26.614 | −41.042 | 42.018 | 1.00 | 60.98 | O |
| ATOM | 3188 | N | ALA | L | 193 | −26.858 | −41.414 | 39.803 | 1.00 | 60.37 | N |
| ATOM | 3189 | CA | ALA | L | 193 | −25.926 | −42.547 | 39.754 | 1.00 | 59.26 | C |
| ATOM | 3190 | CB | ALA | L | 193 | −26.663 | −43.825 | 39.363 | 1.00 | 59.15 | C |
| ATOM | 3191 | C | ALA | L | 193 | −24.752 | −42.317 | 38.811 | 1.00 | 58.42 | C |
| ATOM | 3192 | O | ALA | L | 193 | −24.919 | −41.886 | 37.667 | 1.00 | 58.16 | O |
| ATOM | 3193 | N | CYS | L | 194 | −23.560 | −42.612 | 39.312 | 1.00 | 57.54 | N |
| ATOM | 3194 | CA | CYS | L | 194 | −22.369 | −42.632 | 38.489 | 1.00 | 56.62 | C |
| ATOM | 3195 | CB | CYS | L | 194 | −21.171 | −42.077 | 39.259 | 1.00 | 56.14 | C |
| ATOM | 3196 | SG | CYS | L | 194 | −19.697 | −42.078 | 38.256 | 1.00 | 54.87 | S |
| ATOM | 3197 | C | CYS | L | 194 | −22.113 | −44.069 | 38.056 | 1.00 | 56.54 | C |
| ATOM | 3198 | O | CYS | L | 194 | −21.854 | −44.934 | 38.887 | 1.00 | 56.23 | O |
| ATOM | 3199 | N | GLU | L | 195 | −22.196 | −44.309 | 36.753 | 1.00 | 56.49 | N |
| ATOM | 3200 | CA | GLU | L | 195 | −22.013 | −45.636 | 36.169 | 1.00 | 56.81 | C |
| ATOM | 3201 | CB | GLU | L | 195 | −23.104 | −45.840 | 35.112 | 1.00 | 57.12 | C |
| ATOM | 3202 | CG | GLU | L | 195 | −22.916 | −47.019 | 34.171 | 1.00 | 59.82 | C |
| ATOM | 3203 | CD | GLU | L | 195 | −23.545 | −48.277 | 34.691 | 1.00 | 62.48 | C |
| ATOM | 3204 | OE1 | GLU | L | 195 | −24.508 | −48.760 | 34.053 | 1.00 | 63.10 | O |
| ATOM | 3205 | OE2 | GLU | L | 195 | −23.086 | −48.772 | 35.746 | 1.00 | 65.15 | O |
| ATOM | 3206 | C | GLU | L | 195 | −20.605 | −45.780 | 35.550 | 1.00 | 56.19 | C |
| ATOM | 3207 | O | GLU | L | 195 | −20.230 | −44.982 | 34.700 | 1.00 | 56.61 | O |
| ATOM | 3208 | N | VAL | L | 196 | −19.838 | −46.791 | 35.958 | 1.00 | 55.40 | N |
| ATOM | 3209 | CA | VAL | L | 196 | −18.434 | −46.904 | 35.539 | 1.00 | 55.03 | C |
| ATOM | 3210 | CB | VAL | L | 196 | −17.448 | −46.632 | 36.726 | 1.00 | 54.94 | C |
| ATOM | 3211 | CG1 | VAL | L | 196 | −15.988 | −46.784 | 36.284 | 1.00 | 54.36 | C |
| ATOM | 3212 | CG2 | VAL | L | 196 | −17.665 | −45.246 | 37.322 | 1.00 | 54.09 | C |
| ATOM | 3213 | C | VAL | L | 196 | −18.077 | −48.244 | 34.880 | 1.00 | 55.14 | C |
| ATOM | 3214 | O | VAL | L | 196 | −18.219 | −49.296 | 35.501 | 1.00 | 55.18 | O |
| ATOM | 3215 | N | THR | L | 197 | −17.611 | −48.183 | 33.627 | 1.00 | 55.17 | N |
| ATOM | 3216 | CA | THR | L | 197 | −17.081 | −49.349 | 32.893 | 1.00 | 54.95 | C |
| ATOM | 3217 | CB | THR | L | 197 | −17.734 | −49.523 | 31.503 | 1.00 | 54.81 | C |
| ATOM | 3218 | OG1 | THR | L | 197 | −19.146 | −49.313 | 31.594 | 1.00 | 54.96 | O |
| ATOM | 3219 | CG2 | THR | L | 197 | −17.471 | −50.920 | 30.956 | 1.00 | 55.07 | C |
| ATOM | 3220 | C | THR | L | 197 | −15.569 | −49.233 | 32.694 | 1.00 | 55.00 | C |
| ATOM | 3221 | O | THR | L | 197 | −15.052 | −48.149 | 32.416 | 1.00 | 55.06 | O |
| ATOM | 3222 | N | HIS | L | 198 | −14.879 | −50.363 | 32.824 | 1.00 | 55.08 | N |
| ATOM | 3223 | CA | HIS | L | 198 | −13.428 | −50.420 | 32.790 | 1.00 | 55.30 | C |
| ATOM | 3224 | CB | HIS | L | 198 | −12.859 | −49.796 | 34.068 | 1.00 | 54.79 | C |
| ATOM | 3225 | CG | HIS | L | 198 | −11.369 | −49.670 | 34.065 | 1.00 | 54.21 | C |
| ATOM | 3226 | ND1 | HIS | L | 198 | −10.544 | −50.582 | 34.688 | 1.00 | 52.43 | N |
| ATOM | 3227 | CE1 | HIS | L | 198 | −9.287 | −50.222 | 34.515 | 1.00 | 51.59 | C |
| ATOM | 3228 | NE2 | HIS | L | 198 | −9.265 | −49.113 | 33.799 | 1.00 | 51.79 | N |
| ATOM | 3229 | CD2 | HIS | L | 198 | −10.554 | −48.744 | 33.508 | 1.00 | 52.82 | C |
| ATOM | 3230 | C | HIS | L | 198 | −12.976 | −51.876 | 32.681 | 1.00 | 56.03 | C |
| ATOM | 3231 | O | HIS | L | 198 | −13.639 | −52.775 | 33.208 | 1.00 | 55.91 | O |
| ATOM | 3232 | N | GLN | L | 199 | −11.842 | −52.106 | 32.019 | 1.00 | 56.76 | N |
| ATOM | 3233 | CA | GLN | L | 199 | −11.274 | −53.459 | 31.882 | 1.00 | 57.70 | C |
| ATOM | 3234 | CB | GLN | L | 199 | −9.971 | −53.437 | 31.078 | 1.00 | 57.82 | C |
| ATOM | 3235 | CG | GLN | L | 199 | −10.144 | −53.828 | 29.610 | 1.00 | 59.21 | C |
| ATOM | 3236 | CD | GLN | L | 199 | −8.818 | −53.954 | 28.871 | 1.00 | 61.00 | C |
| ATOM | 3237 | OE1 | GLN | L | 199 | −8.582 | −54.932 | 28.155 | 1.00 | 61.37 | O |
| ATOM | 3238 | NE2 | GLN | L | 199 | −7.946 | −52.964 | 29.044 | 1.00 | 61.05 | N |
| ATOM | 3239 | C | GLN | L | 199 | −11.055 | −54.203 | 33.204 | 1.00 | 57.95 | C |
| ATOM | 3240 | O | GLN | L | 199 | −11.118 | −55.429 | 33.231 | 1.00 | 58.24 | O |
| ATOM | 3241 | N | GLY | L | 200 | −10.809 | −53.466 | 34.286 | 1.00 | 58.22 | N |
| ATOM | 3242 | CA | GLY | L | 200 | −10.601 | −54.058 | 35.615 | 1.00 | 58.75 | C |
| ATOM | 3243 | C | GLY | L | 200 | −11.866 | −54.522 | 36.323 | 1.00 | 59.24 | C |
| ATOM | 3244 | O | GLY | L | 200 | −11.814 | −55.030 | 37.445 | 1.00 | 59.02 | O |
| ATOM | 3245 | N | LEU | L | 201 | −13.006 | −54.342 | 35.668 | 1.00 | 59.65 | N |
| ATOM | 3246 | CA | LEU | L | 201 | −14.289 | −54.742 | 36.232 | 1.00 | 60.19 | C |
| ATOM | 3247 | CB | LEU | L | 201 | −15.153 | −53.505 | 36.539 | 1.00 | 60.01 | C |
| ATOM | 3248 | CG | LEU | L | 201 | −14.585 | −52.455 | 37.499 | 1.00 | 59.80 | C |
| ATOM | 3249 | CD1 | LEU | L | 201 | −15.322 | −51.128 | 37.363 | 1.00 | 60.45 | C |
| ATOM | 3250 | CD2 | LEU | L | 201 | −14.613 | −52.954 | 38.941 | 1.00 | 59.90 | C |
| ATOM | 3251 | C | LEU | L | 201 | −14.996 | −55.631 | 35.228 | 1.00 | 60.37 | C |
| ATOM | 3252 | O | LEU | L | 201 | −15.264 | −55.201 | 34.100 | 1.00 | 60.42 | O |
| ATOM | 3253 | N | SER | L | 202 | −15.285 | −56.866 | 35.633 | 1.00 | 60.93 | N |
| ATOM | 3254 | CA | SER | L | 202 | −16.045 | −57.802 | 34.789 | 1.00 | 61.29 | C |
| ATOM | 3255 | CB | SER | L | 202 | −16.321 | −59.124 | 35.512 | 1.00 | 61.21 | C |
| ATOM | 3256 | OG | SER | L | 202 | −16.483 | −58.924 | 36.905 | 1.00 | 61.50 | O |
| ATOM | 3257 | C | SER | L | 202 | −17.341 | −57.155 | 34.328 | 1.00 | 61.47 | C |
| ATOM | 3258 | O | SER | L | 202 | −17.635 | −57.138 | 33.131 | 1.00 | 61.70 | O |
| ATOM | 3259 | N | SER | L | 203 | −18.078 | −56.590 | 35.287 | 1.00 | 61.54 | N |
| ATOM | 3260 | CA | SER | L | 203 | −19.337 | −55.882 | 35.037 | 1.00 | 61.51 | C |
| ATOM | 3261 | CB | SER | L | 203 | −20.525 | −56.733 | 35.504 | 1.00 | 61.61 | C |
| ATOM | 3262 | OG | SER | L | 203 | −20.309 | −57.230 | 36.818 | 1.00 | 62.00 | O |
| ATOM | 3263 | C | SER | L | 203 | −19.336 | −54.503 | 35.725 | 1.00 | 61.13 | C |
| ATOM | 3264 | O | SER | L | 203 | −18.664 | −54.321 | 36.741 | 1.00 | 61.03 | O |
| ATOM | 3265 | N | PRO | L | 204 | −20.085 | −53.530 | 35.168 | 1.00 | 60.99 | N |
| ATOM | 3266 | CA | PRO | L | 204 | −20.038 | −52.123 | 35.615 | 1.00 | 60.71 | C |
| ATOM | 3267 | CB | PRO | L | 204 | −21.115 | −51.452 | 34.761 | 1.00 | 60.71 | C |
| ATOM | 3268 | CG | PRO | L | 204 | −21.159 | −52.277 | 33.516 | 1.00 | 61.03 | C |
| ATOM | 3269 | CD | PRO | L | 204 | −20.952 | −53.692 | 33.984 | 1.00 | 60.94 | C |
| ATOM | 3270 | C | PRO | L | 204 | −20.316 | −51.861 | 37.100 | 1.00 | 60.57 | C |
| ATOM | 3271 | O | PRO | L | 204 | −21.121 | −52.553 | 37.721 | 1.00 | 60.43 | O |
| ATOM | 3272 | N | VAL | L | 205 | −19.638 | −50.858 | 37.650 | 1.00 | 60.23 | N |
| ATOM | 3273 | CA | VAL | L | 205 | −19.863 | −50.422 | 39.018 | 1.00 | 60.06 | C |
| ATOM | 3274 | CB | VAL | L | 205 | −18.530 | −50.200 | 39.776 | 1.00 | 60.15 | C |
| ATOM | 3275 | CG1 | VAL | L | 205 | −18.728 | −49.335 | 41.017 | 1.00 | 59.95 | C |
| ATOM | 3276 | CG2 | VAL | L | 205 | −17.922 | −51.540 | 40.161 | 1.00 | 60.46 | C |
| ATOM | 3277 | C | VAL | L | 205 | −20.699 | −49.154 | 39.008 | 1.00 | 59.94 | C |
| ATOM | 3278 | O | VAL | L | 205 | −20.357 | −48.181 | 38.339 | 1.00 | 60.07 | O |
| ATOM | 3279 | N | THR | L | 206 | −21.810 | −49.183 | 39.739 | 1.00 | 59.90 | N |
| ATOM | 3280 | CA | THR | L | 206 | −22.665 | −48.012 | 39.894 | 1.00 | 59.62 | C |
| ATOM | 3281 | CB | THR | L | 206 | −24.111 | −48.301 | 39.466 | 1.00 | 59.52 | C |
| ATOM | 3282 | OG1 | THR | L | 206 | −24.110 | −48.979 | 38.204 | 1.00 | 59.00 | O |
| ATOM | 3283 | CG2 | THR | L | 206 | −24.897 | −47.009 | 39.341 | 1.00 | 58.96 | C |
| ATOM | 3284 | C | THR | L | 206 | −22.637 | −47.527 | 41.339 | 1.00 | 59.89 | C |
| ATOM | 3285 | O | THR | L | 206 | −22.823 | −48.305 | 42.272 | 1.00 | 59.73 | O |
| ATOM | 3286 | N | LYS | L | 207 | −22.373 | −46.238 | 41.512 | 1.00 | 60.14 | N |
| ATOM | 3287 | CA | LYS | L | 207 | −22.438 | −45.603 | 42.818 | 1.00 | 60.49 | C |
| ATOM | 3288 | CB | LYS | L | 207 | −21.066 | −45.078 | 43.258 | 1.00 | 60.39 | C |
| ATOM | 3289 | CG | LYS | L | 207 | −19.980 | −46.139 | 43.377 | 1.00 | 60.89 | C |
| ATOM | 3290 | CD | LYS | L | 207 | −20.200 | −47.067 | 44.564 | 1.00 | 61.37 | C |
| ATOM | 3291 | CE | LYS | L | 207 | −18.956 | −47.879 | 44.854 | 1.00 | 61.13 | C |
| ATOM | 3292 | NZ | LYS | L | 207 | −19.090 | −48.641 | 46.121 | 1.00 | 62.42 | N |
| ATOM | 3293 | C | LYS | L | 207 | −23.435 | −44.464 | 42.723 | 1.00 | 60.83 | C |
| ATOM | 3294 | O | LYS | L | 207 | −23.372 | −43.650 | 41.801 | 1.00 | 60.54 | O |
| ATOM | 3295 | N | SER | L | 208 | −24.352 | −44.413 | 43.683 | 1.00 | 61.47 | N |
| ATOM | 3296 | CA | SER | L | 208 | −25.466 | −43.479 | 43.639 | 1.00 | 62.27 | C |
| ATOM | 3297 | CB | SER | L | 208 | −26.695 | −44.167 | 43.046 | 1.00 | 62.04 | C |
| ATOM | 3298 | OG | SER | L | 208 | −26.974 | −45.349 | 43.764 | 1.00 | 63.31 | O |
| ATOM | 3299 | C | SER | L | 208 | −25.820 | −42.897 | 45.006 | 1.00 | 62.72 | C |
| ATOM | 3300 | O | SER | L | 208 | −25.334 | −43.354 | 46.046 | 1.00 | 62.65 | O |
| ATOM | 3301 | N | PHE | L | 209 | −26.656 | −41.864 | 44.972 | 1.00 | 63.50 | N |
| ATOM | 3302 | CA | PHE | L | 209 | −27.285 | −41.290 | 46.154 | 1.00 | 64.34 | C |
| ATOM | 3303 | CB | PHE | L | 209 | −26.442 | −40.147 | 46.751 | 1.00 | 64.25 | C |
| ATOM | 3304 | CG | PHE | L | 209 | −26.383 | −38.909 | 45.882 | 1.00 | 64.26 | C |
| ATOM | 3305 | CD1 | PHE | L | 209 | −27.337 | −37.900 | 46.011 | 1.00 | 63.16 | C |
| ATOM | 3306 | CE1 | PHE | L | 209 | −27.289 | −36.769 | 45.205 | 1.00 | 63.08 | C |
| ATOM | 3307 | CZ | PHE | L | 209 | −26.277 | −36.627 | 44.261 | 1.00 | 62.74 | C |
| ATOM | 3308 | CE2 | PHE | L | 209 | −25.315 | −37.618 | 44.126 | 1.00 | 62.96 | C |
| ATOM | 3309 | CD2 | PHE | L | 209 | −25.371 | −38.755 | 44.932 | 1.00 | 63.86 | C |
| ATOM | 3310 | C | PHE | L | 209 | −28.651 | −40.783 | 45.727 | 1.00 | 65.07 | C |
| ATOM | 3311 | O | PHE | L | 209 | −28.890 | −40.563 | 44.536 | 1.00 | 64.76 | O |
| ATOM | 3312 | N | ASN | L | 210 | −29.549 | −40.619 | 46.694 | 1.00 | 66.46 | N |
| ATOM | 3313 | CA | ASN | L | 210 | −30.831 | −39.953 | 46.451 | 1.00 | 67.80 | C |
| ATOM | 3314 | CB | ASN | L | 210 | −32.012 | −40.815 | 46.909 | 1.00 | 67.68 | C |
| ATOM | 3315 | CG | ASN | L | 210 | −31.890 | −42.262 | 46.457 | 1.00 | 68.02 | C |
| ATOM | 3316 | OD1 | ASN | L | 210 | −32.214 | −42.610 | 45.318 | 1.00 | 67.59 | O |
| ATOM | 3317 | ND2 | ASN | L | 210 | −31.418 | −43.116 | 47.358 | 1.00 | 68.33 | N |
| ATOM | 3318 | C | ASN | L | 210 | −30.833 | −38.594 | 47.143 | 1.00 | 68.61 | C |
| ATOM | 3319 | O | ASN | L | 210 | −30.434 | −38.482 | 48.305 | 1.00 | 68.90 | O |
| ATOM | 3320 | N | ARG | L | 211 | −31.261 | −37.571 | 46.406 | 1.00 | 69.78 | N |
| ATOM | 3321 | CA | ARG | L | 211 | −31.240 | −36.174 | 46.853 | 1.00 | 71.00 | C |
| ATOM | 3322 | CB | ARG | L | 211 | −31.769 | −35.274 | 45.726 | 1.00 | 71.01 | C |
| ATOM | 3323 | CG | ARG | L | 211 | −32.037 | −33.826 | 46.114 | 1.00 | 71.62 | C |
| ATOM | 3324 | CD | ARG | L | 211 | −32.807 | −33.090 | 45.019 | 1.00 | 72.99 | C |
| ATOM | 3325 | NE | ARG | L | 211 | −33.935 | −33.868 | 44.493 | 1.00 | 73.78 | N |
| ATOM | 3326 | CZ | ARG | L | 211 | −35.159 | −33.881 | 45.016 | 1.00 | 74.01 | C |
| ATOM | 3327 | NH1 | ARG | L | 211 | −35.435 | −33.159 | 46.094 | 1.00 | 74.22 | N |
| ATOM | 3328 | NH2 | ARG | L | 211 | −36.109 | −34.622 | 44.460 | 1.00 | 73.79 | N |
| ATOM | 3329 | C | ARG | L | 211 | −32.021 | −35.948 | 48.159 | 1.00 | 71.84 | C |
| ATOM | 3330 | O | ARG | L | 211 | −33.217 | −36.246 | 48.241 | 1.00 | 71.91 | O |
| ATOM | 3331 | N | GLY | L | 212 | −31.330 | −35.425 | 49.172 | 1.00 | 72.70 | N |
| ATOM | 3332 | CA | GLY | L | 212 | −31.939 | −35.178 | 50.480 | 1.00 | 73.83 | C |
| ATOM | 3333 | C | GLY | L | 212 | −31.572 | −36.207 | 51.538 | 1.00 | 74.48 | C |
| ATOM | 3334 | O | GLY | L | 212 | −31.328 | −35.852 | 52.693 | 1.00 | 74.72 | O |
| ATOM | 3335 | N | GLU | L | 213 | −31.537 | −37.480 | 51.146 | 1.00 | 74.94 | N |
| ATOM | 3336 | CA | GLU | L | 213 | −31.207 | −38.579 | 52.065 | 1.00 | 75.44 | C |
| ATOM | 3337 | CB | GLU | L | 213 | −31.842 | −39.888 | 51.585 | 1.00 | 75.56 | C |
| ATOM | 3338 | CG | GLU | L | 213 | −33.370 | −39.844 | 51.478 | 1.00 | 76.26 | C |
| ATOM | 3339 | CD | GLU | L | 213 | −33.921 | −40.816 | 50.436 | 1.00 | 77.49 | C |
| ATOM | 3340 | OE1 | GLU | L | 213 | −33.352 | −41.922 | 50.286 | 1.00 | 78.04 | O |
| ATOM | 3341 | OE2 | GLU | L | 213 | −34.924 | −40.474 | 49.766 | 1.00 | 77.74 | O |
| ATOM | 3342 | C | GLU | L | 213 | −29.694 | −38.760 | 52.224 | 1.00 | 75.52 | C |
| ATOM | 3343 | O | GLU | L | 213 | −29.169 | −38.807 | 53.342 | 1.00 | 75.51 | O |
| TER | 3345 | | GLU | L | 213 | | | | | | |
| ATOM | 3344 | O | HOH | S | 1 | 3.836 | −30.539 | 17.924 | 1.00 | 23.94 | O |
| ATOM | 3345 | O | HOH | S | 2 | 16.318 | −40.515 | 14.150 | 1.00 | 25.15 | O |
| ATOM | 3346 | O | HOH | S | 3 | 8.937 | −26.382 | 7.070 | 1.00 | 26.32 | O |
| ATOM | 3347 | O | HOH | S | 4 | −1.434 | −16.671 | 31.985 | 1.00 | 30.93 | O |
| ATOM | 3348 | O | HOH | S | 5 | 17.890 | −28.116 | 21.969 | 1.00 | 28.52 | O |
| ATOM | 3349 | O | HOH | S | 6 | 12.289 | −27.959 | 14.781 | 1.00 | 30.89 | O |
| ATOM | 3350 | O | HOH | S | 7 | 18.383 | −22.451 | 18.273 | 1.00 | 34.20 | O |
| ATOM | 3351 | O | HOH | S | 8 | 7.394 | −16.965 | 33.250 | 1.00 | 31.25 | O |
| ATOM | 3352 | O | HOH | S | 9 | 5.852 | −11.594 | 30.350 | 1.00 | 44.79 | O |
| ATOM | 3353 | O | HOH | S | 10 | 19.163 | −38.477 | 5.744 | 1.00 | 36.58 | O |
| ATOM | 3354 | O | HOH | S | 11 | 23.171 | −20.413 | 18.960 | 1.00 | 30.69 | O |
| ATOM | 3355 | O | HOH | S | 12 | 10.301 | −38.466 | 33.114 | 1.00 | 33.58 | O |
| ATOM | 3356 | O | HOH | S | 13 | 24.094 | −26.337 | 14.634 | 1.00 | 36.27 | O |
| ATOM | 3357 | O | HOH | S | 14 | 7.609 | −39.155 | 3.488 | 1.00 | 33.76 | O |
| ATOM | 3358 | O | HOH | S | 15 | 4.432 | −15.681 | 36.476 | 1.00 | 38.65 | O |
| ATOM | 3359 | O | HOH | S | 16 | 7.684 | −49.812 | 21.359 | 1.00 | 32.18 | O |
| ATOM | 3360 | O | HOH | S | 17 | −2.723 | −13.281 | 16.878 | 1.00 | 36.37 | O |
| ATOM | 3361 | O | HOH | S | 18 | −8.943 | −18.714 | 29.476 | 1.00 | 24.46 | O |
| ATOM | 3362 | O | HOH | S | 19 | −11.674 | −17.888 | 29.389 | 1.00 | 31.56 | O |
| ATOM | 3363 | O | HOH | S | 20 | 21.891 | −43.297 | 17.995 | 1.00 | 42.63 | O |
| ATOM | 3364 | O | HOH | S | 21 | 13.206 | −11.980 | 20.263 | 1.00 | 32.77 | O |
| ATOM | 3365 | O | HOH | S | 22 | −0.085 | −41.263 | 29.966 | 1.00 | 44.35 | O |
| ATOM | 3366 | O | HOH | S | 23 | 16.929 | −6.803 | 17.430 | 1.00 | 47.48 | O |
| ATOM | 3367 | O | HOH | S | 24 | −5.661 | −19.098 | 32.593 | 1.00 | 45.87 | O |
| ATOM | 3368 | O | HOH | S | 25 | 6.179 | −53.341 | 27.039 | 1.00 | 37.24 | O |
| ATOM | 3369 | O | HOH | S | 26 | −14.935 | −9.438 | 31.335 | 1.00 | 36.62 | O |
| ATOM | 3370 | O | HOH | S | 27 | −20.143 | −31.980 | 44.321 | 1.00 | 34.24 | O |
| ATOM | 3371 | O | HOH | S | 28 | 23.344 | −20.784 | 10.634 | 1.00 | 33.84 | O |
| ATOM | 3372 | O | HOH | S | 29 | 33.157 | −24.532 | 17.646 | 1.00 | 37.02 | O |
| ATOM | 3373 | O | HOH | S | 30 | 11.667 | −35.432 | −0.567 | 1.00 | 34.79 | O |
| ATOM | 3374 | O | HOH | S | 31 | −19.777 | −27.865 | 37.420 | 1.00 | 54.13 | O |
| ATOM | 3375 | O | HOH | S | 32 | 5.110 | −31.743 | 4.504 | 1.00 | 42.40 | O |
| ATOM | 3376 | O | HOH | S | 33 | −9.815 | −14.241 | 19.878 | 1.00 | 41.72 | O |
| ATOM | 3377 | O | HOH | S | 34 | 9.081 | −54.632 | 34.766 | 1.00 | 33.37 | O |
| ATOM | 3378 | O | HOH | S | 35 | −8.650 | −11.635 | 30.376 | 1.00 | 49.69 | O |
| ATOM | 3379 | O | HOH | S | 36 | 5.373 | −41.907 | 11.390 | 1.00 | 42.52 | O |
| ATOM | 3380 | O | HOH | S | 37 | 26.530 | −30.513 | 0.242 | 1.00 | 42.24 | O |
| ATOM | 3381 | O | HOH | S | 38 | 8.065 | −52.735 | 24.788 | 1.00 | 38.10 | O |
| ATOM | 3382 | O | HOH | S | 39 | −3.456 | −46.136 | 29.846 | 1.00 | 41.05 | O |
| ATOM | 3383 | O | HOH | S | 40 | 4.821 | −52.023 | 35.674 | 1.00 | 29.48 | O |
| ATOM | 3384 | O | HOH | S | 41 | 20.357 | −44.204 | 10.602 | 1.00 | 46.94 | O |
| ATOM | 3385 | O | HOH | S | 42 | 4.816 | −36.474 | 8.118 | 1.00 | 38.25 | O |
| ATOM | 3386 | O | HOH | S | 43 | 21.524 | −27.456 | 13.643 | 1.00 | 43.38 | O |
| ATOM | 3387 | O | HOH | S | 44 | −3.307 | −38.587 | 31.749 | 1.00 | 35.46 | O |
| ATOM | 3388 | O | HOH | S | 45 | 5.308 | −34.344 | 29.735 | 1.00 | 47.93 | O |
| ATOM | 3389 | O | HOH | S | 46 | 16.655 | −25.508 | 3.332 | 1.00 | 36.51 | O |
| ATOM | 3390 | O | HOH | S | 47 | −3.112 | −5.344 | 19.720 | 1.00 | 49.22 | O |
| ATOM | 3391 | O | HOH | S | 48 | 18.098 | −24.962 | 29.998 | 1.00 | 40.67 | O |
| ATOM | 3392 | O | HOH | S | 49 | −14.926 | −33.773 | 29.275 | 1.00 | 43.96 | O |
| ATOM | 3393 | O | HOH | S | 50 | 4.724 | −18.151 | 33.137 | 1.00 | 35.19 | O |
| ATOM | 3394 | O | HOH | S | 51 | 11.954 | −32.492 | 29.705 | 1.00 | 43.62 | O |
| ATOM | 3395 | O | HOH | S | 52 | −18.573 | −26.108 | 30.733 | 1.00 | 45.32 | O |
| ATOM | 3396 | O | HOH | S | 53 | −19.182 | −29.883 | 45.753 | 1.00 | 38.85 | O |
| ATOM | 3397 | O | HOH | S | 54 | −1.343 | −44.100 | 19.538 | 1.00 | 36.30 | O |
| ATOM | 3398 | O | HOH | S | 55 | 28.433 | −32.359 | 16.146 | 1.00 | 37.19 | O |
| ATOM | 3399 | O | HOH | S | 56 | −3.202 | −18.345 | 17.108 | 1.00 | 43.11 | O |
| ATOM | 3400 | O | HOH | S | 57 | −23.263 | −36.228 | 30.943 | 1.00 | 49.63 | O |
| ATOM | 3401 | O | HOH | S | 58 | 25.882 | −32.741 | 0.206 | 1.00 | 37.91 | O |
| ATOM | 3402 | O | HOH | S | 59 | −12.846 | −32.163 | 58.557 | 1.00 | 52.46 | O |
| ATOM | 3403 | O | HOH | S | 60 | 9.378 | −25.249 | 4.513 | 1.00 | 42.31 | O |
| ATOM | 3404 | O | HOH | S | 61 | 23.082 | −27.196 | 26.632 | 1.00 | 40.02 | O |
| ATOM | 3405 | O | HOH | S | 62 | 26.113 | −40.643 | 19.361 | 1.00 | 36.87 | O |
| ATOM | 3406 | O | HOH | S | 63 | 2.927 | −38.757 | 32.669 | 1.00 | 51.01 | O |
| ATOM | 3407 | O | HOH | S | 64 | −7.049 | −25.674 | 31.536 | 1.00 | 49.27 | O |
| ATOM | 3408 | O | HOH | S | 65 | 5.326 | −25.205 | 14.196 | 1.00 | 34.27 | O |
| ATOM | 3409 | O | HOH | S | 66 | 19.449 | −32.987 | 27.073 | 1.00 | 39.04 | O |
| ATOM | 3410 | O | HOH | S | 67 | −20.257 | −47.072 | 32.217 | 1.00 | 46.48 | O |
| ATOM | 3411 | O | HOH | S | 68 | 18.944 | −47.015 | 17.097 | 1.00 | 40.67 | O |
| ATOM | 3412 | O | HOH | S | 69 | 5.504 | −27.738 | 14.000 | 1.00 | 40.72 | O |
| ATOM | 3413 | O | HOH | S | 70 | 25.619 | −19.249 | 18.405 | 1.00 | 38.01 | O |
| ATOM | 3414 | O | HOH | S | 71 | −10.555 | −47.084 | 58.137 | 1.00 | 40.09 | O |
| ATOM | 3415 | O | HOH | S | 72 | 23.151 | −37.470 | 10.674 | 1.00 | 39.45 | O |
| ATOM | 3416 | O | HOH | S | 73 | −2.560 | −41.827 | 52.012 | 1.00 | 41.59 | O |
| ATOM | 3417 | O | HOH | S | 74 | 1.676 | −53.740 | 37.660 | 1.00 | 39.32 | O |
| ATOM | 3418 | O | HOH | S | 75 | 6.068 | −48.749 | 19.050 | 1.00 | 44.41 | O |
| ATOM | 3419 | O | HOH | S | 76 | −15.612 | −21.144 | 26.981 | 1.00 | 41.71 | O |
| ATOM | 3420 | O | HOH | S | 77 | −0.815 | −9.713 | 31.904 | 1.00 | 38.48 | O |
| ATOM | 3421 | O | HOH | S | 78 | −1.076 | −47.351 | 22.758 | 1.00 | 39.28 | O |
| ATOM | 3422 | O | HOH | S | 79 | −5.497 | −41.105 | 46.341 | 1.00 | 46.26 | O |
| ATOM | 3423 | O | HOH | S | 80 | 18.448 | −47.764 | 22.046 | 1.00 | 43.53 | O |
| ATOM | 3424 | O | HOH | S | 81 | 11.184 | −7.976 | 12.259 | 1.00 | 47.49 | O |
| ATOM | 3425 | O | HOH | S | 82 | −7.996 | −33.228 | 32.150 | 1.00 | 51.36 | O |
| ATOM | 3426 | O | HOH | S | 83 | −4.658 | −26.850 | 36.047 | 1.00 | 48.36 | O |
| ATOM | 3427 | O | HOH | S | 84 | 4.078 | −32.321 | 28.279 | 1.00 | 48.93 | O |
| ATOM | 3428 | O | HOH | S | 85 | 13.451 | −24.547 | 31.156 | 1.00 | 45.87 | O |
| ATOM | 3429 | O | HOH | S | 86 | −5.041 | −25.963 | 57.076 | 1.00 | 64.63 | O |
| ATOM | 3430 | O | HOH | S | 87 | −10.823 | −30.308 | 36.508 | 1.00 | 40.84 | O |
| ATOM | 3431 | O | HOH | S | 88 | −6.716 | −11.829 | 34.980 | 1.00 | 42.68 | O |
| ATOM | 3432 | O | HOH | S | 89 | 0.580 | −13.968 | 9.893 | 1.00 | 54.54 | O |
| ATOM | 3433 | O | HOH | S | 90 | 15.522 | −20.899 | 33.304 | 1.00 | 52.55 | O |
| ATOM | 3434 | O | HOH | S | 91 | 3.590 | −28.603 | 10.814 | 1.00 | 41.24 | O |
| ATOM | 3435 | O | HOH | S | 92 | −5.246 | −38.854 | 57.212 | 1.00 | 42.94 | O |
| ATOM | 3436 | O | HOH | S | 93 | −5.241 | −24.010 | 32.695 | 1.00 | 58.18 | O |
| ATOM | 3437 | O | HOH | S | 94 | −12.741 | −8.479 | 37.655 | 1.00 | 48.64 | O |
| ATOM | 3438 | O | HOH | S | 95 | 7.079 | −25.843 | 32.170 | 1.00 | 54.89 | O |
| ATOM | 3439 | O | HOH | S | 96 | −20.258 | −22.923 | 32.708 | 1.00 | 47.80 | O |
| ATOM | 3440 | O | HOH | S | 97 | −4.455 | −22.119 | 22.435 | 1.00 | 39.45 | O |
| ATOM | 3441 | O | HOH | S | 98 | 4.245 | −26.110 | 28.406 | 1.00 | 52.48 | O |
| ATOM | 3442 | O | HOH | S | 99 | 21.381 | −47.689 | 23.468 | 1.00 | 45.80 | O |
| ATOM | 3443 | O | HOH | S | 100 | 22.968 | −36.078 | 6.064 | 1.00 | 43.18 | O |
| ATOM | 3444 | O | HOH | S | 101 | −8.276 | −48.638 | 42.470 | 1.00 | 49.72 | O |
| ATOM | 3445 | O | HOH | S | 102 | 7.864 | −10.366 | 34.466 | 1.00 | 46.44 | O |
| ATOM | 3446 | O | HOH | S | 103 | 4.707 | −34.151 | 0.708 | 1.00 | 46.62 | O |
| ATOM | 3447 | O | HOH | S | 104 | −0.062 | −27.225 | 17.512 | 1.00 | 63.91 | O |
| ATOM | 3448 | O | HOH | S | 105 | 6.151 | −19.959 | 7.514 | 1.00 | 45.39 | O |
| ATOM | 3449 | O | HOH | S | 106 | −9.780 | −41.685 | 30.513 | 1.00 | 57.14 | O |
| ATOM | 3450 | O | HOH | S | 107 | 18.123 | −45.272 | 7.011 | 1.00 | 53.99 | O |
| ATOM | 3451 | O | HOH | S | 108 | −7.647 | −29.823 | 31.984 | 1.00 | 49.82 | O |
| ATOM | 3452 | O | HOH | S | 109 | −2.441 | −6.332 | 14.720 | 1.00 | 47.89 | O |
| ATOM | 3453 | O | HOH | S | 110 | 26.025 | −27.904 | 24.272 | 1.00 | 50.59 | O |
| ATOM | 3454 | O | HOH | S | 111 | 27.201 | −24.434 | 26.361 | 1.00 | 58.51 | O |
| ATOM | 3455 | O | HOH | S | 112 | −29.894 | −37.925 | 56.557 | 1.00 | 77.81 | O |
| ATOM | 3456 | O | HOH | S | 113 | −2.729 | −17.192 | 14.270 | 1.00 | 53.84 | O |
| ATOM | 3457 | O | HOH | S | 114 | −27.619 | −39.267 | 56.443 | 1.00 | 61.03 | O |
| ATOM | 3458 | O | HOH | S | 115 | −2.161 | −46.630 | 25.104 | 1.00 | 38.70 | O |
| ATOM | 3459 | O | HOH | S | 116 | −26.384 | −35.499 | 59.925 | 1.00 | 72.50 | O |
| ATOM | 3460 | O | HOH | S | 117 | −13.917 | −42.466 | 61.582 | 1.00 | 53.93 | O |
| ATOM | 3461 | O | HOH | S | 118 | 28.317 | −32.265 | 13.384 | 1.00 | 42.98 | O |
| ATOM | 3462 | O | HOH | S | 119 | 20.424 | −24.205 | 30.263 | 1.00 | 62.62 | O |
| ATOM | 3463 | O | HOH | S | 120 | −8.325 | −54.373 | 39.925 | 1.00 | 51.56 | O |
| ATOM | 3464 | O | HOH | S | 121 | −2.581 | −30.021 | 45.390 | 1.00 | 45.87 | O |
| ATOM | 3465 | O | HOH | S | 122 | −3.669 | −43.210 | 20.028 | 1.00 | 44.62 | O |
| ATOM | 3466 | O | HOH | S | 123 | 26.063 | −40.619 | 14.495 | 1.00 | 50.70 | O |
| ATOM | 3467 | O | HOH | S | 124 | −12.524 | −18.858 | 26.998 | 1.00 | 36.60 | O |
| ATOM | 3468 | O | HOH | S | 125 | −21.123 | −28.092 | 45.893 | 1.00 | 58.19 | O |
| ATOM | 3469 | O | HOH | S | 126 | −2.167 | −33.924 | 42.562 | 1.00 | 41.23 | O |
| ATOM | 3470 | O | HOH | S | 127 | −14.074 | −12.203 | 42.565 | 1.00 | 53.07 | O |
| ATOM | 3471 | O | HOH | S | 128 | −1.703 | −46.815 | 18.917 | 1.00 | 40.92 | O |
| ATOM | 3472 | O | HOH | S | 129 | −7.994 | −18.819 | 26.800 | 1.00 | 41.60 | O |
| ATOM | 3473 | O | HOH | S | 130 | −8.995 | −48.452 | 59.564 | 1.00 | 39.16 | O |
| ATOM | 3474 | O | HOH | S | 131 | 2.046 | −23.636 | 12.557 | 1.00 | 55.52 | O |
| ATOM | 3475 | O | HOH | S | 132 | 13.747 | −22.843 | 4.089 | 1.00 | 42.50 | O |
| ATOM | 3476 | O | HOH | S | 133 | 11.995 | −18.641 | 5.160 | 1.00 | 54.22 | O |
| ATOM | 3477 | O | HOH | S | 134 | −4.335 | −24.477 | 38.893 | 1.00 | 48.54 | O |
| ATOM | 3478 | O | HOH | S | 135 | −14.345 | −30.950 | 39.408 | 1.00 | 41.67 | O |
| ATOM | 3479 | O | HOH | S | 136 | −20.367 | −25.804 | 33.629 | 1.00 | 57.40 | O |
| ATOM | 3480 | O | HOH | S | 137 | −9.391 | −56.070 | 38.278 | 1.00 | 59.63 | O |
| ATOM | 3481 | O | HOH | S | 138 | −28.480 | −41.056 | 55.044 | 1.00 | 55.79 | O |
| ATOM | 3482 | O | HOH | S | 139 | 2.349 | −33.011 | 12.392 | 1.00 | 52.84 | O |
| ATOM | 3483 | O | HOH | S | 140 | 5.375 | −26.822 | 30.477 | 1.00 | 60.15 | O |
| ATOM | 3484 | O | HOH | S | 141 | 24.625 | −8.571 | 24.451 | 1.00 | 60.94 | O |
| ATOM | 3485 | O | HOH | S | 142 | −16.775 | −41.641 | 29.614 | 1.00 | 49.84 | O |
| ATOM | 3486 | O | HOH | S | 143 | −0.984 | −24.149 | 40.942 | 1.00 | 54.40 | O |
| ATOM | 3487 | O | HOH | S | 144 | 20.391 | −40.358 | 7.812 | 1.00 | 45.97 | O |
| ATOM | 3488 | O | HOH | S | 145 | −1.468 | −34.651 | 37.114 | 1.00 | 51.88 | O |
| ATOM | 3489 | O | HOH | S | 146 | 6.681 | −9.840 | 36.586 | 1.00 | 49.41 | O |
| ATOM | 3490 | O | HOH | S | 147 | −4.537 | −28.378 | 39.019 | 1.00 | 51.13 | O |
| ATOM | 3491 | O | HOH | S | 148 | 0.181 | −37.932 | 22.036 | 1.00 | 52.82 | O |
| ATOM | 3492 | O | HOH | S | 149 | 26.565 | −33.653 | 22.185 | 1.00 | 41.73 | O |
| ATOM | 3493 | O | HOH | S | 150 | −1.398 | −16.435 | 34.784 | 1.00 | 41.74 | O |
| ATOM | 3494 | O | HOH | S | 151 | 8.146 | −55.541 | 25.532 | 1.00 | 65.57 | O |
| ATOM | 3495 | O | HOH | S | 152 | 0.964 | −6.673 | 10.892 | 1.00 | 59.98 | O |
| ATOM | 3496 | O | HOH | S | 153 | 13.657 | −33.864 | 35.353 | 1.00 | 59.45 | O |
| ATOM | 3497 | O | HOH | S | 154 | 15.317 | −10.993 | 33.514 | 1.00 | 45.63 | O |
| ATOM | 3498 | O | HOH | S | 155 | −0.910 | −33.896 | 26.620 | 1.00 | 67.42 | O |
| ATOM | 3499 | O | HOH | S | 156 | −16.773 | −40.401 | 49.946 | 1.00 | 48.85 | O |
| ATOM | 3500 | O | HOH | S | 157 | 6.741 | −26.243 | 1.044 | 1.00 | 54.82 | O |
| ATOM | 3501 | O | HOH | S | 158 | 4.837 | −55.837 | 27.616 | 1.00 | 61.67 | O |
| ATOM | 3502 | O | HOH | S | 159 | 21.713 | −49.931 | 25.079 | 1.00 | 48.98 | O |
| ATOM | 3503 | O | HOH | S | 160 | −22.817 | −27.809 | 52.873 | 1.00 | 46.68 | O |
| TER | 3506 | | HOH | S | 160 | | | | | | |
| ATOM | 3504 | S | SO4 | C | 1 | 25.580 | −28.568 | 12.061 | 1.00 | 40.05 | S |
| ATOM | 3505 | O1 | SO4 | C | 1 | 25.207 | −27.962 | 10.792 | 1.00 | 41.68 | O |
| ATOM | 3506 | O2 | SO4 | C | 1 | 26.671 | −29.514 | 11.916 | 1.00 | 41.25 | O |
| ATOM | 3507 | O3 | SO4 | C | 1 | 24.484 | −29.365 | 12.551 | 1.00 | 44.54 | O |
| ATOM | 3508 | O4 | SO4 | C | 1 | 25.907 | −27.502 | 13.000 | 1.00 | 43.39 | O |
| TER | 3512 | | SO4 | C | 1 | | | | | | |
| ATOM | 3509 | S | SO4 | C | 2 | 28.273 | −21.403 | 17.397 | 1.00 | 65.92 | S |
| ATOM | 3510 | O1 | SO4 | C | 2 | 27.796 | −21.367 | 16.023 | 1.00 | 67.22 | O |
| ATOM | 3511 | O2 | SO4 | C | 2 | 28.745 | −22.756 | 17.679 | 1.00 | 65.25 | O |
| ATOM | 3512 | O3 | SO4 | C | 2 | 27.173 | −21.054 | 18.295 | 1.00 | 67.63 | O |
| ATOM | 3513 | O4 | SO4 | C | 2 | 29.331 | −20.412 | 17.607 | 1.00 | 67.04 | O |
| TER | 3518 | | SO4 | C | 2 | | | | | | |
| ATOM | 3514 | O4 | PGE | C | 3 | 17.369 | −23.211 | 5.685 | 1.00 | 53.72 | O |
| ATOM | 3515 | C6 | PGE | C | 3 | 17.503 | −22.829 | 7.067 | 1.00 | 53.67 | C |
| ATOM | 3516 | C5 | PGE | C | 3 | 18.935 | −22.988 | 7.576 | 1.00 | 53.51 | C |
| ATOM | 3517 | O3 | PGE | C | 3 | 19.394 | −21.744 | 8.099 | 1.00 | 53.83 | O |
| ATOM | 3518 | C4 | PGE | C | 3 | 20.392 | −21.890 | 9.103 | 1.00 | 52.29 | C |
| ATOM | 3519 | C3 | PGE | C | 3 | 19.981 | −21.119 | 10.347 | 1.00 | 52.29 | C |
| ATOM | 3520 | O2 | PGE | C | 3 | 19.975 | −19.720 | 10.077 | 1.00 | 54.60 | O |
| ATOM | 3521 | C2 | PGE | C | 3 | 20.598 | −18.964 | 11.125 | 1.00 | 53.79 | C |
| ATOM | 3522 | C1 | PGE | C | 3 | 21.058 | −17.584 | 10.659 | 1.00 | 53.65 | C |
| ATOM | 3523 | O1 | PGE | C | 3 | 22.463 | −17.426 | 10.954 | 1.00 | 55.51 | O |
| TER | 3529 | | PGE | C | 3 | | | | | | |
| END | | | | | | | | | | | |

Structure Solution, Model Building, and Refinement: Fab:LPA(14:0) Complex. Merged scalepack output were converted to MTZ format using scalepack2mtz. The CCP4 suite: programs for protein crystallography (1994). Acta Crystallogr. D. Biol. Crystallogr. 50:760-763. Molecular replacement was carried out in phaser using apo Fab as a starting model. McCoy, A. J. et al. (2007) J. Appl Crystallogr 40:658:674. Further refinement by maximum likelihood methods was run in REFMAC5. Murshudov G N, Vagin A A, Dodson E J (1997) Acta Crystallogr. D. Biol. Crystallogr. 53: 240-255. The model was rebuilt in the program Coot. Emsley, P. et al., (2010) Acta Crystallogr. D. Biol. Crystallogr. 66:486-501.

The model was then further built in the program PHENIX. Adams, P. D. et al. (2010) Acta Crystallogr. D. Biol. Crystallogr. 66:213:221. Coordinates for lysophosphatidic acid were prepared using the PRODRG server. Schuttelkopf A. W, van Aalten DM (2004) Acta Crystallogr. D. Biol. Crystallogr. 60:1355-1363. A library file was prepared via Monomer Library Sketcher. The CCP4 suite: programs for protein crystallography (1994). Acta Crystallogr. D. Biol. Crystallogr. 50:760-763. Stereochemical analysis and final adjustments to the model were directed by MOLPROBITY. Davis I W et al., (2007) Nucl. Acids. Res. 35:W375-383. Final adjustments were made in Coot. Emsley, P. et al., (2010) Acta Crystallogr. D. Biol. Crystallogr. 66:486-501.

The coordinates for the Fab:LPA(14:0) crystal are provided as Table 20, below.

TABLE 20
LT3015Fab:LPA (14:0) co-crystal x-ray coordinates at 1.98 Å resolution

| HEADER | --- XX-XXX-XX xxxx | |
|---|---|---|
| COMPND | — | |
| REMARK | 3 | REFINEMENT. |
| REMARK | 3 | PROGRAM: REFMAC 5.5.0109 |
| REMARK | 3 | REFINEMENT TARGET: MAXIMUM LIKELIHOOD |
| REMARK | 3 | DATA USED IN REFINEMENT. |
| REMARK | 3 | RESOLUTION RANGE HIGH (ANGSTROMS): 1.98 |
| REMARK | 3 | RESOLUTION RANGE LOW (ANGSTROMS): 92.18 |
| REMARK | 3 | DATA CUTOFF (SIGMA(F)): NONE |
| REMARK | 3 | COMPLETENESS FOR RANGE (%): 99.89 |
| REMARK | 3 | NUMBER OF REFLECTIONS: 68835 |
| REMARK | 3 | FIT TO DATA USED IN REFINEMENT. |
| REMARK | 3 | CROSS-VALIDATION METHOD: THROUGHOUT |
| REMARK | 3 | FREE R VALUE TEST SET SELECTION: RANDOM |
| REMARK | 3 | R VALUE (WORKING + TEST SET): 0.22304 |
| REMARK | 3 | R VALUE (WORKING SET): 0.22151 |
| REMARK | 3 | FREE R VALUE: 0.25161 |
| REMARK | 3 | FREE R VALUE TEST SET SIZE (%): 5.0 |
| REMARK | 3 | FREE R VALUE TEST SET COUNT: 3653 |
| REMARK | 3 | FIT IN THE HIGHEST RESOLUTION BIN. |
| REMARK | 3 | TOTAL NUMBER OF BINS USED: 20 |
| REMARK | 3 | BIN RESOLUTION RANGE HIGH: 1.979 |
| REMARK | 3 | BIN RESOLUTION RANGE LOW: 2.031 |
| REMARK | 3 | REFLECTION IN BIN (WORKING SET): 5017 |
| REMARK | 3 | BIN COMPLETENESS (WORKING + TEST) (%): 99.53 |
| REMARK | 3 | BIN R VALUE (WORKING SET): 0.247 |
| REMARK | 3 | BIN FREE R VALUE SET COUNT: 275 |
| REMARK | 3 | BIN FREE R VALUE: 0.309 |
| REMARK | 3 | NUMBER OF NON-HYDROGEN ATOMS USED IN REFINEMENT. |
| REMARK | 3 | ALL ATOMS: 7020 |
| REMARK | 3 | B VALUES. |
| REMARK | 3 | FROM WILSON PLOT (A**2): NULL |
| REMARK | 3 | MEAN B VALUE (OVERALL, A**2): 22.730 |
| REMARK | 3 | OVERALL ANISOTROPIC B VALUE. |
| REMARK | 3 | B11 (A**2): −0.46 |
| REMARK | 3 | B22 (A**2): −0.51 |
| REMARK | 3 | B33 (A**2): 0.98 |
| REMARK | 3 | B12 (A**2): 0.00 |
| REMARK | 3 | B13 (A**2): 0.00 |
| REMARK | 3 | B23 (A**2): 0.00 |
| REMARK | 3 | ESTIMATED OVERALL COORDINATE ERROR. |
| REMARK | 3 | ESU BASED ON R VALUE (A): 0.185 |
| REMARK | 3 | ESU BASED ON FREE R VALUE (A): 0.162 |
| REMARK | 3 | ESU BASED ON MAXIMUM LIKELIHOOD (A): 0.122 |
| REMARK | 3 | ESU FOR B VALUES BASED ON MAXIMUM LIKELIHOOD (A**2): 9.329 |
| REMARK | 3 | CORRELATION COEFFICIENTS. |
| REMARK | 3 | CORRELATION COEFFICIENT FO-FC: 0.945 |
| REMARK | 3 | CORRELATION COEFFICIENT FO-FC FREE: 0.926 |
| REMARK | 3 | RMS DEVIATIONS FROM IDEAL VALUES COUNT RMS WEIGHT |
| REMARK | 3 | BOND LENGTHS REFINED ATOMS (A): 6910; 0.010; 0.022 |
| REMARK | 3 | BOND ANGLES REFINED ATOMS (DEGREES): 9390; 1.288; 1.957 |
| REMARK | 3 | TORSION ANGLES, PERIOD 1 (DEGREES): 868; 6.374; 5.000 |
| REMARK | 3 | TORSION ANGLES, PERIOD 2 (DEGREES): 276; 35.781; 24.710 |
| REMARK | 3 | TORSION ANGLES, PERIOD 3 (DEGREES): 1088; 13.692; 15.000 |
| REMARK | 3 | TORSION ANGLES, PERIOD 4 (DEGREES): 18; 22.426; 15.000 |
| REMARK | 3 | CHIRAL-CENTER RESTRAINTS (A**3): 1034; 0.080; 0.200 |
| REMARK | 3 | GENERAL PLANES REFINED ATOMS (A): 5202; 0.005; 0.021 |
| REMARK | 3 | ISOTROPIC THERMAL FACTOR RESTRAINTS. COUNT RMS WEIGHT |
| REMARK | 3 | MAIN-CHAIN BOND REFINED ATOMS (A**2): 4340; 0.619; 1.500 |
| REMARK | 3 | MAIN-CHAIN ANGLE REFINED ATOMS (A**2): 7020; 1.144; 2.000 |
| REMARK | 3 | SIDE-CHAIN BOND REFINED ATOMS (A**2): 2570; 1.713; 3.000 |
| REMARK | 3 | SIDE-CHAIN ANGLE REFINED ATOMS (A**2): 2370; 2.757; 4.500 |
| REMARK | 3 | NCS RESTRAINTS STATISTICS |
| REMARK | 3 | TWIN DETAILS |
| REMARK | 3 | NUMBER OF TWIN DOMAINS: NULL |
| REMARK | 3 | TLS DETAILS |
| REMARK | 3 | NUMBER OF TLS GROUPS: 4 |
| REMARK | 3 | ATOM RECORD CONTAINS RESIDUAL B FACTORS ONLY |
| REMARK | 3 | TLS GROUP: 1 |
| REMARK | 3 | NUMBER OF COMPONENTS GROUP: 1 |
| REMARK | 3 | COMPONENTS C SSSEQI TO C SSSEQI |
| REMARK | 3 | RESIDUE RANGE: H −10 H 9999 |
| REMARK | 3 | ORIGIN FOR THE GROUP (A): 32.9087 −34.2621 13.0430 |
| REMARK | 3 | T TENSOR |
| REMARK | 3 | T11: 0.0598 T22: 0.1073 |
| REMARK | 3 | T33: 0.0276 T12: −0.0178 |
| REMARK | 3 | T13: 0.0069 T23: −0.0085 |
| REMARK | 3 | L TENSOR |
| REMARK | 3 | L11: 2.6433 L22: 1.1439 |
| REMARK | 3 | L33: 0.7806 L12: −1.2744 |
| REMARK | 3 | L13: 0.1308 L23: −0.0123 |
| REMARK | 3 | S TENSOR |
| REMARK | 3 | S11: −0.0014 S12: 0.0257 S13: −0.1268 |
| REMARK | 3 | S21: 0.0192 S22: 0.0015 S23: 0.0994 |
| REMARK | 3 | S31: 0.0358 S32: −0.2797 S33: −0.0001 |
| REMARK | 3 | TLS GROUP: 2 |
| REMARK | 3 | NUMBER OF COMPONENTS GROUP: 1 |
| REMARK | 3 | COMPONENTS C SSSEQI TO C SSSEQI |
| REMARK | 3 | RESIDUE RANGE: L −10 L 9999 |
| REMARK | 3 | ORIGIN FOR THE GROUP (A): 37.5167 −18.8294 21.6458 |
| REMARK | 3 | T TENSOR |
| REMARK | 3 | T11: 0.0433 T22: 0.1266 |
| REMARK | 3 | T33: 0.0424 T12: 0.0341 |
| REMARK | 3 | T13: 0.0079 T23: −0.0069 |
| REMARK | 3 | L TENSOR |
| REMARK | 3 | L11: 2.2473 L22: 0.3583 |
| REMARK | 3 | L33: 0.9348 L12: −0.4714 |
| REMARK | 3 | L13: −0.1583 L23: 0.3358 |
| REMARK | 3 | S TENSOR |
| REMARK | 3 | S11: 0.0436 S12: −0.1535 S13: 0.1473 |
| REMARK | 3 | S21: 0.0329 S22: −0.0411 S23: 0.0229 |
| REMARK | 3 | S31: −0.0657 S32: −0.3071 S33: −0.0025 |
| REMARK | 3 | TLS GROUP: 3 |
| REMARK | 3 | NUMBER OF COMPONENTS GROUP: 1 |
| REMARK | 3 | COMPONENTS C SSSEQI TO C SSSEQI |
| REMARK | 3 | RESIDUE RANGE: I −10 I 9999 |
| REMARK | 3 | ORIGIN FOR THE GROUP (A): 49.6404 −31.6494 −12.8709 |
| REMARK | 3 | T TENSOR |
| REMARK | 3 | T11: 0.1055 T22: 0.0342 |
| REMARK | 3 | T33: 0.0600 T12: −0.0045 |
| REMARK | 3 | T13: 0.0523 T23: −0.0200 |
| REMARK | 3 | L TENSOR |
| REMARK | 3 | L11: 2.1063 L22: 1.1094 |
| REMARK | 3 | L33: 0.4954 L12: 1.0528 |
| REMARK | 3 | L13: 0.4307 L23: 0.2212 |
| REMARK | 3 | S TENSOR |
| REMARK | 3 | S11: −0.0390 S12: 0.0459 S13: −0.0796 |
| REMARK | 3 | S21: 0.0003 S22: 0.0391 S23: −0.1467 |
| REMARK | 3 | S31: 0.0485 S32: 0.0943 S33: −0.0001 |
| REMARK | 3 | TLS GROUP: 4 |
| REMARK | 3 | NUMBER OF COMPONENTS GROUP: 1 |
| REMARK | 3 | COMPONENTS C SSSEQI TO C SSSEQI |
| REMARK | 3 | RESIDUE RANGE: M −10 M 9999 |
| REMARK | 3 | ORIGIN FOR THE GROUP (A): 47.0607 −14.1229 −16.8001 |
| REMARK | 3 | T TENSOR |
| REMARK | 3 | T11: 0.1485 T22: 0.0405 |
| REMARK | 3 | T33: 0.0424 T12: −0.0144 |
| REMARK | 3 | T13: 0.0274 T23: −0.0014 |
| REMARK | 3 | L TENSOR |
| REMARK | 3 | L11: 2.9536 L22: 0.8324 |
| REMARK | 3 | L33: 0.4115 L12: 0.8510 |
| REMARK | 3 | L13: −0.2053 L23: −0.3265 |
| REMARK | 3 | S TENSOR |
| REMARK | 3 | S11: −0.0290 S12: 0.2469 S13: 0.0543 |
| REMARK | 3 | S21: −0.1297 S22: 0.0192 S23: −0.0478 |
| REMARK | 3 | S31: 0.0283 S32: 0.0631 S33: 0.0098 |
| REMARK | 3 | BULK SOLVENT MODELLING. |
| REMARK | 3 | METHOD USED: MASK |
| REMARK | 3 | PARAMETERS FOR MASK CALCULATION |
| REMARK | 3 | VDW PROBE RADIUS: 1.40 |
| REMARK | 3 | ION PROBE RADIUS: 0.80 |
| REMARK | 3 | SHRINKAGE RADIUS: 0.80 |
| REMARK | 3 | OTHER REFINEMENT REMARKS: |
| REMARK | 3 | HYDROGENS HAVE BEEN ADDED IN THE RIDING POSITIONS |
| REMARK | 3 | U VALUES: RESIDUAL ONLY |
| SSBOND | 1 | CYS H 22 CYS H 92 |
| SSBOND | 2 | CYS H 140 CYS H 196 |
| SSBOND | 3 | CYS L 23 CYS L 88 |
| SSBOND | 4 | CYS L 134 CYS L 194 |
| SSBOND | 5 | CYS I 22 CYS I 92 |
| SSBOND | 6 | CYS I 140 CYS I 196 |
| SSBOND | 7 | CYS M 23 CYS M 88 |
| SSBOND | 8 | CYS M 134 CYS M 194 |

| | |
|---|---|
| LINKR | SER H 128 GLY H 133 gap |
| CISPEP | 1 PHE H 146 PRO H 147 0.00 |
| CISPEP | 2 GLU H 148 PRO H 149 0.00 |
| CISPEP | 3 THR L 7 PRO L 8 0.00 |
| CISPEP | 4 PHE L 94 PRO L 95 0.00 |
| CISPEP | 5 TYR L 140 PRO L 141 0.00 |
| LINKR | ALA H 137 VAL H 142 gap |
| LINKR | SER I 128 GLY I 133 gap |
| CISPEP | 6 PHE I 146 PRO I 147 0.00 |
| CISPEP | 7 GLU I 148 PRO I 149 0.00 |
| LINKR | ALA I 137 VAL I 142 gap |
| CISPEP | 8 THR M 7 PRO M 8 0.00 |
| CISPEP | 9 PHE M 94 PRO M 95 0.00 |
| CISPEP | 10 TYR M 140 PRO M 141 0.00 |
| CRYST1 | 86.526 184.351 130.000 90.00 90.00 90.00 C 2 2 21 |
| SCALE1 | 0.011557 0.000000 0.000000 0.00000 |
| SCALE2 | 0.000000 0.005424 0.000000 0.00000 |
| SCALE3 | 0.000000 0.000000 0.007692 0.00000 |

ATOMS and Details

| | | | | | | | | | | | |
|---|---|---|---|---|---|---|---|---|---|---|---|
| ATOM | 1 | N | GLU | H | 1 | 51.089 | −36.445 | −4.933 | 1.00 | 41.62 | N |
| ATOM | 2 | CA | GLU | H | 1 | 50.292 | −36.100 | −3.716 | 1.00 | 41.28 | C |
| ATOM | 3 | CB | GLU | H | 1 | 49.124 | −35.155 | −4.073 | 1.00 | 41.80 | C |
| ATOM | 4 | CG | GLU | H | 1 | 48.422 | −34.499 | −2.854 | 1.00 | 43.06 | C |
| ATOM | 5 | CD | GLU | H | 1 | 46.970 | −34.083 | −3.114 | 1.00 | 44.79 | C |
| ATOM | 6 | OE1 | GLU | H | 1 | 46.120 | −34.302 | −2.212 | 1.00 | 46.27 | O |
| ATOM | 7 | OE2 | GLU | H | 1 | 46.667 | −33.537 | −4.200 | 1.00 | 44.63 | O |
| ATOM | 8 | C | GLU | H | 1 | 49.777 | −37.364 | −2.998 | 1.00 | 40.67 | C |
| ATOM | 9 | O | GLU | H | 1 | 49.026 | −38.168 | −3.583 | 1.00 | 40.95 | O |
| ATOM | 10 | N | VAL | H | 2 | 50.194 | −37.524 | −1.736 | 1.00 | 39.16 | N |
| ATOM | 11 | CA | VAL | H | 2 | 49.657 | −38.546 | −0.827 | 1.00 | 37.23 | C |
| ATOM | 12 | CB | VAL | H | 2 | 50.719 | −39.018 | 0.213 | 1.00 | 37.48 | C |
| ATOM | 13 | CG1 | VAL | H | 2 | 50.275 | −40.283 | 0.921 | 1.00 | 38.39 | C |
| ATOM | 14 | CG2 | VAL | H | 2 | 52.094 | −39.239 | −0.452 | 1.00 | 38.39 | C |
| ATOM | 15 | C | VAL | H | 2 | 48.412 | −38.000 | −0.108 | 1.00 | 35.49 | C |
| ATOM | 16 | O | VAL | H | 2 | 48.383 | −36.830 | 0.305 | 1.00 | 35.28 | O |
| ATOM | 17 | N | GLN | H | 3 | 47.398 | −38.862 | 0.032 | 1.00 | 32.88 | N |
| ATOM | 18 | CA | GLN | H | 3 | 46.100 | −38.537 | 0.624 | 1.00 | 29.96 | C |
| ATOM | 19 | CB | GLN | H | 3 | 45.051 | −38.441 | −0.496 | 1.00 | 30.59 | C |
| ATOM | 20 | CG | GLN | H | 3 | 43.821 | −37.572 | −0.197 | 1.00 | 33.01 | C |
| ATOM | 21 | CD | GLN | H | 3 | 42.929 | −37.342 | −1.425 | 1.00 | 34.72 | C |
| ATOM | 22 | OE1 | GLN | H | 3 | 43.401 | −37.310 | −2.574 | 1.00 | 35.79 | O |
| ATOM | 23 | NE2 | GLN | H | 3 | 41.633 | −37.171 | −1.182 | 1.00 | 34.77 | N |
| ATOM | 24 | C | GLN | H | 3 | 45.745 | −39.683 | 1.575 | 1.00 | 27.43 | C |
| ATOM | 25 | O | GLN | H | 3 | 46.146 | −40.828 | 1.351 | 1.00 | 26.69 | O |
| ATOM | 26 | N | LEU | H | 4 | 45.010 | −39.372 | 2.632 | 1.00 | 23.99 | N |
| ATOM | 27 | CA | LEU | H | 4 | 44.487 | −40.370 | 3.561 | 1.00 | 21.83 | C |
| ATOM | 28 | CB | LEU | H | 4 | 45.117 | −40.212 | 4.965 | 1.00 | 21.13 | C |
| ATOM | 29 | CG | LEU | H | 4 | 46.640 | −40.461 | 5.093 | 1.00 | 19.42 | C |
| ATOM | 30 | CD1 | LEU | H | 4 | 47.242 | −39.994 | 6.470 | 1.00 | 18.51 | C |
| ATOM | 31 | CD2 | LEU | H | 4 | 46.991 | −41.921 | 4.846 | 1.00 | 16.80 | C |
| ATOM | 32 | C | LEU | H | 4 | 42.985 | −40.154 | 3.604 | 1.00 | 21.04 | C |
| ATOM | 33 | O | LEU | H | 4 | 42.539 | −39.040 | 3.859 | 1.00 | 20.16 | O |
| ATOM | 34 | N | VAL | H | 5 | 42.207 | −41.196 | 3.313 | 1.00 | 19.30 | N |
| ATOM | 35 | CA | VAL | H | 5 | 40.737 | −41.092 | 3.363 | 1.00 | 18.63 | C |
| ATOM | 36 | CB | VAL | H | 5 | 40.077 | −41.438 | 2.014 | 1.00 | 18.28 | C |
| ATOM | 37 | CG1 | VAL | H | 5 | 38.567 | −41.267 | 2.095 | 1.00 | 19.16 | C |
| ATOM | 38 | CG2 | VAL | H | 5 | 40.644 | −40.573 | 0.896 | 1.00 | 18.67 | C |
| ATOM | 39 | C | VAL | H | 5 | 40.143 | −41.971 | 4.457 | 1.00 | 18.15 | C |
| ATOM | 40 | O | VAL | H | 5 | 40.345 | −43.203 | 4.477 | 1.00 | 17.66 | O |
| ATOM | 41 | N | GLN | H | 6 | 39.398 | −41.339 | 5.355 | 1.00 | 18.18 | N |
| ATOM | 42 | CA | GLN | H | 6 | 38.839 | −42.043 | 6.503 | 1.00 | 18.73 | C |
| ATOM | 43 | CB | GLN | H | 6 | 38.977 | −41.203 | 7.759 | 1.00 | 19.02 | C |
| ATOM | 44 | CG | GLN | H | 6 | 40.427 | −40.835 | 8.058 | 1.00 | 19.18 | C |
| ATOM | 45 | CD | GLN | H | 6 | 40.581 | −40.085 | 9.367 | 1.00 | 19.10 | C |
| ATOM | 46 | OE1 | GLN | H | 6 | 41.232 | −39.037 | 9.418 | 1.00 | 19.56 | O |
| ATOM | 47 | NE2 | GLN | H | 6 | 40.012 | −40.628 | 10.438 | 1.00 | 18.94 | N |
| ATOM | 48 | C | GLN | H | 6 | 37.381 | −42.428 | 6.282 | 1.00 | 19.31 | C |
| ATOM | 49 | O | GLN | H | 6 | 36.682 | −41.815 | 5.472 | 1.00 | 19.19 | O |
| ATOM | 50 | N | SER | H | 7 | 36.925 | −43.434 | 7.021 | 1.00 | 19.37 | N |
| ATOM | 51 | CA | SER | H | 7 | 35.542 | −43.904 | 6.903 | 1.00 | 20.22 | C |
| ATOM | 52 | CB | SER | H | 7 | 35.387 | −45.281 | 7.577 | 1.00 | 20.31 | C |
| ATOM | 53 | OG | SER | H | 7 | 35.949 | −45.265 | 8.885 | 1.00 | 19.93 | O |
| ATOM | 54 | C | SER | H | 7 | 34.557 | −42.861 | 7.457 | 1.00 | 20.49 | C |
| ATOM | 55 | O | SER | H | 7 | 34.963 | −41.917 | 8.145 | 1.00 | 21.19 | O |
| ATOM | 56 | N | GLY | H | 8 | 33.273 | −43.034 | 7.146 | 1.00 | 21.64 | N |
| ATOM | 57 | CA | GLY | H | 8 | 32.235 | −42.054 | 7.467 | 1.00 | 22.17 | C |
| ATOM | 58 | C | GLY | H | 8 | 31.934 | −41.923 | 8.954 | 1.00 | 23.22 | C |
| ATOM | 59 | O | GLY | H | 8 | 32.342 | −42.774 | 9.759 | 1.00 | 23.76 | O |
| ATOM | 60 | N | ALA | H | 9 | 31.228 | −40.849 | 9.307 | 1.00 | 22.95 | N |
| ATOM | 61 | CA | ALA | H | 9 | 30.808 | −40.564 | 10.682 | 1.00 | 23.66 | C |
| ATOM | 62 | CB | ALA | H | 9 | 29.937 | −39.311 | 10.719 | 1.00 | 23.53 | C |
| ATOM | 63 | C | ALA | H | 9 | 30.079 | −41.747 | 11.313 | 1.00 | 23.41 | C |
| ATOM | 64 | O | ALA | H | 9 | 29.342 | −42.471 | 10.634 | 1.00 | 23.62 | O |
| ATOM | 65 | N | GLU | H | 10 | 30.307 | −41.951 | 12.609 | 1.00 | 23.25 | N |
| ATOM | 66 | CA | GLU | H | 10 | 29.759 | −43.107 | 13.329 | 1.00 | 23.28 | C |
| ATOM | 67 | CB | GLU | H | 10 | 30.893 | −43.986 | 13.870 | 1.00 | 23.80 | C |
| ATOM | 68 | CG | GLU | H | 10 | 31.811 | −44.587 | 12.828 | 1.00 | 25.69 | C |
| ATOM | 69 | CD | GLU | H | 10 | 31.469 | −46.028 | 12.473 | 1.00 | 28.85 | C |
| ATOM | 70 | OE1 | GLU | H | 10 | 30.412 | −46.548 | 12.906 | 1.00 | 30.17 | O |
| ATOM | 71 | OE2 | GLU | H | 10 | 32.274 | −46.651 | 11.752 | 1.00 | 30.20 | O |
| ATOM | 72 | C | GLU | H | 10 | 28.955 | −42.622 | 14.522 | 1.00 | 22.89 | C |
| ATOM | 73 | O | GLU | H | 10 | 29.389 | −41.723 | 15.245 | 1.00 | 22.07 | O |
| ATOM | 74 | N | VAL | H | 11 | 27.798 | −43.232 | 14.736 | 1.00 | 22.26 | N |
| ATOM | 75 | CA | VAL | H | 11 | 27.027 | −43.011 | 15.961 | 1.00 | 22.16 | C |
| ATOM | 76 | CB | VAL | H | 11 | 25.641 | −42.397 | 15.671 | 1.00 | 22.29 | C |
| ATOM | 77 | CG1 | VAL | H | 11 | 24.993 | −41.895 | 16.956 | 1.00 | 22.67 | C |
| ATOM | 78 | CG2 | VAL | H | 11 | 25.776 | −41.256 | 14.685 | 1.00 | 22.95 | C |
| ATOM | 79 | C | VAL | H | 11 | 26.906 | −44.334 | 16.724 | 1.00 | 21.28 | C |
| ATOM | 80 | O | VAL | H | 11 | 26.453 | −45.347 | 16.178 | 1.00 | 20.72 | O |
| ATOM | 81 | N | LYS | H | 12 | 27.329 | −44.318 | 17.986 | 1.00 | 20.43 | N |
| ATOM | 82 | CA | LYS | H | 12 | 27.431 | −45.539 | 18.779 | 1.00 | 19.98 | C |
| ATOM | 83 | CB | LYS | H | 12 | 28.900 | −45.914 | 18.989 | 1.00 | 19.97 | C |
| ATOM | 84 | CG | LYS | H | 12 | 29.601 | −46.436 | 17.739 | 1.00 | 21.76 | C |
| ATOM | 85 | CD | LYS | H | 12 | 29.246 | −47.906 | 17.549 | 1.00 | 25.35 | C |
| ATOM | 86 | CE | LYS | H | 12 | 29.194 | −48.266 | 16.095 | 1.00 | 26.75 | C |
| ATOM | 87 | NZ | LYS | H | 12 | 28.937 | −49.709 | 15.942 | 1.00 | 28.45 | N |
| ATOM | 88 | C | LYS | H | 12 | 26.758 | −45.381 | 20.138 | 1.00 | 19.57 | C |
| ATOM | 89 | O | LYS | H | 12 | 26.677 | −44.278 | 20.675 | 1.00 | 18.63 | O |
| ATOM | 90 | N | LYS | H | 13 | 26.290 | −46.490 | 20.690 | 1.00 | 18.83 | N |
| ATOM | 91 | CA | LYS | H | 13 | 25.755 | −46.481 | 22.044 | 1.00 | 19.52 | C |
| ATOM | 92 | CB | LYS | H | 13 | 24.612 | −47.489 | 22.167 | 1.00 | 19.02 | C |
| ATOM | 93 | CG | LYS | H | 13 | 23.371 | −47.072 | 21.387 | 1.00 | 20.36 | C |
| ATOM | 94 | CD | LYS | H | 13 | 22.150 | −47.883 | 21.797 | 1.00 | 22.80 | C |
| ATOM | 95 | CE | LYS | H | 13 | 20.856 | −47.054 | 21.703 | 1.00 | 24.55 | C |
| ATOM | 96 | NZ | LYS | H | 13 | 20.600 | −46.488 | 20.347 | 1.00 | 25.05 | N |
| ATOM | 97 | C | LYS | H | 13 | 26.876 | −46.792 | 23.028 | 1.00 | 19.55 | C |
| ATOM | 98 | O | LYS | H | 13 | 27.816 | −47.497 | 22.659 | 1.00 | 19.58 | O |
| ATOM | 99 | N | PRO | H | 14 | 26.798 | −46.252 | 24.271 | 1.00 | 20.22 | N |
| ATOM | 100 | CA | PRO | H | 14 | 27.745 | −46.590 | 25.346 | 1.00 | 20.31 | C |
| ATOM | 101 | CB | PRO | H | 14 | 27.052 | −46.057 | 26.599 | 1.00 | 20.31 | C |
| ATOM | 102 | CG | PRO | H | 14 | 26.310 | −44.874 | 26.126 | 1.00 | 21.44 | C |
| ATOM | 103 | CD | PRO | H | 14 | 25.844 | −45.215 | 24.712 | 1.00 | 20.72 | C |
| ATOM | 104 | C | PRO | H | 14 | 27.918 | −48.095 | 25.486 | 1.00 | 20.42 | C |
| ATOM | 105 | O | PRO | H | 14 | 26.936 | −48.830 | 25.481 | 1.00 | 19.76 | O |
| ATOM | 106 | N | GLY | H | 15 | 29.159 | −48.550 | 25.600 | 1.00 | 20.74 | N |
| ATOM | 107 | CA | GLY | H | 15 | 29.411 | −49.978 | 25.760 | 1.00 | 20.88 | C |
| ATOM | 108 | C | GLY | H | 15 | 29.718 | −50.717 | 24.468 | 1.00 | 21.04 | C |
| ATOM | 109 | O | GLY | H | 15 | 30.285 | −51.810 | 24.504 | 1.00 | 20.70 | O |
| ATOM | 110 | N | GLU | H | 16 | 29.357 | −50.124 | 23.329 | 1.00 | 21.05 | N |
| ATOM | 111 | CA | GLU | H | 16 | 29.643 | −50.719 | 22.026 | 1.00 | 21.23 | C |
| ATOM | 112 | CB | GLU | H | 16 | 28.687 | −50.174 | 20.954 | 1.00 | 21.56 | C |
| ATOM | 113 | CG | GLU | H | 16 | 27.240 | −50.692 | 21.130 | 1.00 | 21.74 | C |
| ATOM | 114 | CD | GLU | H | 16 | 26.237 | −50.132 | 20.116 | 1.00 | 22.25 | C |
| ATOM | 115 | OE1 | GLU | H | 16 | 25.073 | −50.591 | 20.140 | 1.00 | 23.11 | O |
| ATOM | 116 | OE2 | GLU | H | 16 | 26.589 | −49.239 | 19.316 | 1.00 | 22.09 | O |
| ATOM | 117 | C | GLU | H | 16 | 31.099 | −50.537 | 21.598 | 1.00 | 21.56 | C |
| ATOM | 118 | O | GLU | H | 16 | 31.869 | −49.830 | 22.248 | 1.00 | 21.39 | O |
| ATOM | 119 | N | SER | H | 17 | 31.469 | −51.206 | 20.509 | 1.00 | 21.57 | N |
| ATOM | 120 | CA | SER | H | 17 | 32.792 | −51.079 | 19.919 | 1.00 | 21.54 | C |
| ATOM | 121 | CB | SER | H | 17 | 33.413 | −52.465 | 19.680 | 1.00 | 21.75 | C |
| ATOM | 122 | OG | SER | H | 17 | 33.575 | −53.191 | 20.888 | 1.00 | 23.47 | O |
| ATOM | 123 | C | SER | H | 17 | 32.681 | −50.348 | 18.595 | 1.00 | 21.39 | C |
| ATOM | 124 | O | SER | H | 17 | 31.600 | −50.268 | 18.021 | 1.00 | 21.06 | O |
| ATOM | 125 | N | LEU | H | 18 | 33.798 | −49.796 | 18.124 | 1.00 | 21.09 | N |
| ATOM | 126 | CA | LEU | H | 18 | 33.875 | −49.284 | 16.756 | 1.00 | 21.33 | C |
| ATOM | 127 | CB | LEU | H | 18 | 33.705 | −47.752 | 16.721 | 1.00 | 21.51 | C |
| ATOM | 128 | CG | LEU | H | 18 | 34.703 | −46.808 | 17.396 | 1.00 | 21.98 | C |
| ATOM | 129 | CD1 | LEU | H | 18 | 35.955 | −46.608 | 16.566 | 1.00 | 23.12 | C |
| ATOM | 130 | CD2 | LEU | H | 18 | 34.019 | −45.470 | 17.552 | 1.00 | 23.56 | C |
| ATOM | 131 | C | LEU | H | 18 | 35.174 | −49.715 | 16.061 | 1.00 | 20.97 | C |
| ATOM | 132 | O | LEU | H | 18 | 36.140 | −50.107 | 16.715 | 1.00 | 20.53 | O |
| ATOM | 133 | N | LYS | H | 19 | 35.169 | −49.658 | 14.736 | 1.00 | 21.07 | N |
| ATOM | 134 | CA | LYS | H | 19 | 36.354 | −49.881 | 13.922 | 1.00 | 21.52 | C |
| ATOM | 135 | CB | LYS | H | 19 | 36.446 | −51.337 | 13.436 | 1.00 | 21.76 | C |
| ATOM | 136 | CG | LYS | H | 19 | 37.774 | −51.684 | 12.729 | 1.00 | 21.46 | C |
| ATOM | 137 | CD | LYS | H | 19 | 37.868 | −53.187 | 12.391 | 1.00 | 24.22 | C |
| ATOM | 138 | CE | LYS | H | 19 | 39.322 | −53.594 | 12.091 | 1.00 | 24.93 | C |
| ATOM | 139 | NZ | LYS | H | 19 | 39.507 | −55.053 | 11.842 | 1.00 | 26.60 | N |
| ATOM | 140 | C | LYS | H | 19 | 36.265 | −48.902 | 12.747 | 1.00 | 22.14 | C |
| ATOM | 141 | O | LYS | H | 19 | 35.333 | −48.962 | 11.950 | 1.00 | 22.15 | O |
| ATOM | 142 | N | ILE | H | 20 | 37.212 | −47.974 | 12.672 | 1.00 | 21.91 | N |
| ATOM | 143 | CA | ILE | H | 20 | 37.207 | −46.967 | 11.612 | 1.00 | 21.93 | C |
| ATOM | 144 | CB | ILE | H | 20 | 37.021 | −45.537 | 12.185 | 1.00 | 22.26 | C |
| ATOM | 145 | CG1 | ILE | H | 20 | 38.164 | −45.173 | 13.132 | 1.00 | 21.90 | C |
| ATOM | 146 | CD1 | ILE | H | 20 | 37.988 | −43.839 | 13.831 | 1.00 | 22.41 | C |
| ATOM | 147 | CG2 | ILE | H | 20 | 35.675 | −45.438 | 12.913 | 1.00 | 21.93 | C |
| ATOM | 148 | C | ILE | H | 20 | 38.474 | −47.097 | 10.767 | 1.00 | 22.17 | C |
| ATOM | 149 | O | ILE | H | 20 | 39.510 | −47.568 | 11.261 | 1.00 | 21.41 | O |
| ATOM | 150 | N | SER | H | 21 | 38.388 | −46.713 | 9.494 | 1.00 | 21.58 | N |
| ATOM | 151 | CA | SER | H | 21 | 39.497 | −46.956 | 8.576 | 1.00 | 21.56 | C |
| ATOM | 152 | CB | SER | H | 21 | 39.036 | −47.788 | 7.388 | 1.00 | 21.47 | C |
| ATOM | 153 | OG | SER | H | 21 | 38.046 | −47.090 | 6.675 | 1.00 | 22.22 | O |
| ATOM | 154 | C | SER | H | 21 | 40.188 | −45.691 | 8.090 | 1.00 | 21.27 | C |
| ATOM | 155 | O | SER | H | 21 | 39.634 | −44.581 | 8.147 | 1.00 | 20.96 | O |
| ATOM | 156 | N | CYS | H | 22 | 41.415 | −45.884 | 7.630 | 1.00 | 20.78 | N |
| ATOM | 157 | CA | CYS | H | 22 | 42.256 | −44.822 | 7.122 | 1.00 | 21.36 | C |
| ATOM | 158 | CB | CYS | H | 22 | 43.272 | −44.392 | 8.171 | 1.00 | 21.25 | C |
| ATOM | 159 | SG | CYS | H | 22 | 44.429 | −43.134 | 7.579 | 1.00 | 22.94 | S |
| ATOM | 160 | C | CYS | H | 22 | 42.970 | −45.391 | 5.926 | 1.00 | 21.47 | C |
| ATOM | 161 | O | CYS | H | 22 | 43.939 | −46.144 | 6.063 | 1.00 | 21.76 | O |
| ATOM | 162 | N | GLN | H | 23 | 42.484 | −45.032 | 4.749 | 1.00 | 21.49 | N |
| ATOM | 163 | CA | GLN | H | 23 | 42.958 | −45.595 | 3.509 | 1.00 | 21.83 | C |
| ATOM | 164 | CB | GLN | H | 23 | 41.779 | −45.687 | 2.543 | 1.00 | 21.65 | C |
| ATOM | 165 | CG | GLN | H | 23 | 42.114 | −46.298 | 1.212 | 1.00 | 25.31 | C |
| ATOM | 166 | CD | GLN | H | 23 | 42.290 | −47.791 | 1.305 | 1.00 | 30.91 | C |
| ATOM | 167 | OE1 | GLN | H | 23 | 41.423 | −48.506 | 1.840 | 1.00 | 32.66 | O |
| ATOM | 168 | NE2 | GLN | H | 23 | 43.412 | −48.285 | 0.779 | 1.00 | 30.65 | N |
| ATOM | 169 | C | GLN | H | 23 | 44.069 | −44.732 | 2.900 | 1.00 | 22.35 | C |
| ATOM | 170 | O | GLN | H | 23 | 43.887 | −43.530 | 2.704 | 1.00 | 21.18 | O |
| ATOM | 171 | N | ALA | H | 24 | 45.211 | −45.343 | 2.606 | 1.00 | 23.11 | N |
| ATOM | 172 | CA | ALA | H | 24 | 46.316 | −44.616 | 1.985 | 1.00 | 25.70 | C |
| ATOM | 173 | CB | ALA | H | 24 | 47.639 | −44.989 | 2.636 | 1.00 | 25.17 | C |
| ATOM | 174 | C | ALA | H | 24 | 46.385 | −44.828 | 0.472 | 1.00 | 27.17 | C |
| ATOM | 175 | O | ALA | H | 24 | 45.950 | −45.855 | −0.041 | 1.00 | 28.17 | O |
| ATOM | 176 | N | PHE | H | 25 | 46.898 | −43.828 | −0.235 | 1.00 | 29.48 | N |
| ATOM | 177 | CA | PHE | H | 25 | 47.134 | −43.905 | −1.680 | 1.00 | 31.21 | C |
| ATOM | 178 | CB | PHE | H | 25 | 46.143 | −43.050 | −2.469 | 1.00 | 31.24 | C |
| ATOM | 179 | CG | PHE | H | 25 | 44.730 | −43.226 | −2.052 | 1.00 | 30.42 | C |
| ATOM | 180 | CD1 | PHE | H | 25 | 43.864 | −43.993 | −2.810 | 1.00 | 30.70 | C |
| ATOM | 181 | CE1 | PHE | H | 25 | 42.552 | −44.156 | −2.411 | 1.00 | 29.75 | C |
| ATOM | 182 | CZ | PHE | H | 25 | 42.111 | −43.559 | −1.255 | 1.00 | 28.61 | C |
| ATOM | 183 | CE2 | PHE | H | 25 | 42.966 | −42.795 | −0.499 | 1.00 | 29.09 | C |
| ATOM | 184 | CD2 | PHE | H | 25 | 44.259 | −42.631 | −0.894 | 1.00 | 29.95 | C |
| ATOM | 185 | C | PHE | H | 25 | 48.536 | −43.384 | −1.947 | 1.00 | 32.85 | C |
| ATOM | 186 | O | PHE | H | 25 | 48.831 | −42.195 | −1.720 | 1.00 | 33.90 | O |
| ATOM | 187 | N | GLY | H | 26 | 49.381 | −44.294 | −2.412 | 1.00 | 33.76 | N |
| ATOM | 188 | CA | GLY | H | 26 | 50.756 | −44.014 | −2.792 | 1.00 | 35.32 | C |
| ATOM | 189 | C | GLY | H | 26 | 51.474 | −45.319 | −2.550 | 1.00 | 35.90 | C |
| ATOM | 190 | O | GLY | H | 26 | 50.998 | −46.143 | −1.754 | 1.00 | 37.04 | O |
| ATOM | 191 | N | TYR | H | 27 | 52.598 | −45.539 | −3.224 | 1.00 | 35.63 | N |
| ATOM | 192 | CA | TYR | H | 27 | 53.395 | −46.720 | −2.934 | 1.00 | 35.32 | C |
| ATOM | 193 | CB | TYR | H | 27 | 54.467 | −46.941 | −4.003 | 1.00 | 35.97 | C |
| ATOM | 194 | CG | TYR | H | 27 | 53.927 | −47.492 | −5.316 | 1.00 | 38.95 | C |
| ATOM | 195 | CD1 | TYR | H | 27 | 54.153 | −48.828 | −5.681 | 1.00 | 41.06 | C |
| ATOM | 196 | CE1 | TYR | H | 27 | 53.664 | −49.345 | −6.895 | 1.00 | 43.48 | C |
| ATOM | 197 | CZ | TYR | H | 27 | 52.933 | −48.515 | −7.749 | 1.00 | 44.02 | C |
| ATOM | 198 | OH | TYR | H | 27 | 52.443 | −49.011 | −8.944 | 1.00 | 45.17 | O |
| ATOM | 199 | CE2 | TYR | H | 27 | 52.694 | −47.183 | −7.402 | 1.00 | 43.08 | C |
| ATOM | 200 | CD2 | TYR | H | 27 | 53.190 | −46.680 | −6.193 | 1.00 | 41.31 | C |
| ATOM | 201 | C | TYR | H | 27 | 54.031 | −46.537 | −1.561 | 1.00 | 33.95 | C |
| ATOM | 202 | O | TYR | H | 27 | 54.108 | −45.412 | −1.048 | 1.00 | 34.02 | O |
| ATOM | 203 | N | GLY | H | 28 | 54.443 | −47.647 | −0.959 | 1.00 | 32.46 | N |
| ATOM | 204 | CA | GLY | H | 28 | 55.302 | −47.613 | 0.225 | 1.00 | 30.78 | C |
| ATOM | 205 | C | GLY | H | 28 | 54.589 | −47.531 | 1.560 | 1.00 | 29.53 | C |
| ATOM | 206 | O | GLY | H | 28 | 55.194 | −47.123 | 2.556 | 1.00 | 29.02 | O |
| ATOM | 207 | N | PHE | H | 29 | 53.317 | −47.936 | 1.574 | 1.00 | 28.04 | N |
| ATOM | 208 | CA | PHE | H | 29 | 52.494 | −48.011 | 2.791 | 1.00 | 26.70 | C |
| ATOM | 209 | CB | PHE | H | 29 | 51.164 | −48.709 | 2.461 | 1.00 | 26.73 | C |
| ATOM | 210 | CG | PHE | H | 29 | 50.230 | −48.880 | 3.639 | 1.00 | 25.54 | C |
| ATOM | 211 | CD1 | PHE | H | 29 | 49.471 | −47.812 | 4.110 | 1.00 | 25.03 | C |
| ATOM | 212 | CE1 | PHE | H | 29 | 48.591 | −47.971 | 5.180 | 1.00 | 25.16 | C |
| ATOM | 213 | CZ | PHE | H | 29 | 48.446 | −49.215 | 5.769 | 1.00 | 23.87 | C |
| ATOM | 214 | CE2 | PHE | H | 29 | 49.187 | −50.294 | 5.306 | 1.00 | 24.65 | C |
| ATOM | 215 | CD2 | PHE | H | 29 | 50.071 | −50.127 | 4.241 | 1.00 | 24.95 | C |
| ATOM | 216 | C | PHE | H | 29 | 53.215 | −48.749 | 3.922 | 1.00 | 26.16 | C |
| ATOM | 217 | O | PHE | H | 29 | 53.066 | −48.406 | 5.103 | 1.00 | 25.49 | O |
| ATOM | 218 | N | ILE | H | 30 | 54.008 | −49.749 | 3.557 | 1.00 | 25.16 | N |
| ATOM | 219 | CA | ILE | H | 30 | 54.679 | −50.590 | 4.549 | 1.00 | 25.12 | C |
| ATOM | 220 | CB | ILE | H | 30 | 55.320 | −51.864 | 3.929 | 1.00 | 26.01 | C |
| ATOM | 221 | CG1 | ILE | H | 30 | 56.288 | −51.502 | 2.781 | 1.00 | 26.69 | C |
| ATOM | 222 | CD1 | ILE | H | 30 | 57.298 | −52.586 | 2.442 | 1.00 | 28.96 | C |
| ATOM | 223 | CG2 | ILE | H | 30 | 54.224 | −52.852 | 3.498 | 1.00 | 26.04 | C |
| ATOM | 224 | C | ILE | H | 30 | 55.691 | −49.825 | 5.382 | 1.00 | 24.30 | C |
| ATOM | 225 | O | ILE | H | 30 | 55.989 | −50.211 | 6.510 | 1.00 | 23.80 | O |
| ATOM | 226 | N | ASN | H | 31 | 56.203 | −48.725 | 4.837 | 1.00 | 23.84 | N |
| ATOM | 227 | CA | ASN | H | 31 | 57.166 | −47.899 | 5.576 | 1.00 | 24.25 | C |
| ATOM | 228 | CB | ASN | H | 31 | 58.366 | −47.522 | 4.696 | 1.00 | 24.75 | C |
| ATOM | 229 | CG | ASN | H | 31 | 59.312 | −48.692 | 4.484 | 1.00 | 26.44 | C |
| ATOM | 230 | OD1 | ASN | H | 31 | 59.852 | −49.249 | 5.444 | 1.00 | 29.46 | O |
| ATOM | 231 | ND2 | ASN | H | 31 | 59.515 | −49.068 | 3.231 | 1.00 | 26.97 | N |
| ATOM | 232 | C | ASN | H | 31 | 56.559 | −46.667 | 6.235 | 1.00 | 23.64 | C |
| ATOM | 233 | O | ASN | H | 31 | 57.279 | −45.880 | 6.845 | 1.00 | 24.00 | O |
| ATOM | 234 | N | TYR | H | 32 | 55.234 | −46.521 | 6.141 | 1.00 | 22.46 | N |
| ATOM | 235 | CA | TYR | H | 32 | 54.546 | −45.396 | 6.758 | 1.00 | 21.61 | C |
| ATOM | 236 | CB | TYR | H | 32 | 53.071 | −45.372 | 6.368 | 1.00 | 21.68 | C |
| ATOM | 237 | CG | TYR | H | 32 | 52.719 | −44.797 | 4.999 | 1.00 | 23.21 | C |
| ATOM | 238 | CD1 | TYR | H | 32 | 53.663 | −44.699 | 3.983 | 1.00 | 24.62 | C |
| ATOM | 239 | CE1 | TYR | H | 32 | 53.324 | −44.194 | 2.717 | 1.00 | 27.05 | C |
| ATOM | 240 | CZ | TYR | H | 32 | 52.025 | −43.790 | 2.467 | 1.00 | 27.84 | C |
| ATOM | 241 | OH | TYR | H | 32 | 51.684 | −43.298 | 1.224 | 1.00 | 31.58 | O |
| ATOM | 242 | CE2 | TYR | H | 32 | 51.064 | −43.865 | 3.463 | 1.00 | 27.70 | C |
| ATOM | 243 | CD2 | TYR | H | 32 | 51.413 | −44.376 | 4.725 | 1.00 | 26.18 | C |
| ATOM | 244 | C | TYR | H | 32 | 54.620 | −45.478 | 8.275 | 1.00 | 20.80 | C |
| ATOM | 245 | O | TYR | H | 32 | 54.753 | −46.570 | 8.849 | 1.00 | 19.38 | O |
| ATOM | 246 | N | LEU | H | 33 | 54.520 | −44.311 | 8.909 | 1.00 | 19.30 | N |
| ATOM | 247 | CA | LEU | H | 33 | 54.247 | −44.232 | 10.334 | 1.00 | 19.43 | C |
| ATOM | 248 | CB | LEU | H | 33 | 55.364 | −43.461 | 11.077 | 1.00 | 18.64 | C |
| ATOM | 249 | CG | LEU | H | 33 | 56.781 | −44.047 | 10.923 | 1.00 | 19.91 | C |
| ATOM | 250 | CD1 | LEU | H | 33 | 57.767 | −43.206 | 11.701 | 1.00 | 17.75 | C |
| ATOM | 251 | CD2 | LEU | H | 33 | 56.840 | −45.505 | 11.396 | 1.00 | 18.98 | C |
| ATOM | 252 | C | LEU | H | 33 | 52.911 | −43.527 | 10.453 | 1.00 | 18.68 | C |
| ATOM | 253 | O | LEU | H | 33 | 52.810 | −42.333 | 10.199 | 1.00 | 19.90 | O |
| ATOM | 254 | N | ILE | H | 34 | 51.880 | −44.289 | 10.783 | 1.00 | 19.63 | N |
| ATOM | 255 | CA | ILE | H | 34 | 50.535 | −43.759 | 10.877 | 1.00 | 19.16 | C |
| ATOM | 256 | CB | ILE | H | 34 | 49.501 | −44.723 | 10.224 | 1.00 | 19.21 | C |
| ATOM | 257 | CG1 | ILE | H | 34 | 49.731 | −44.836 | 8.716 | 1.00 | 19.15 | C |
| ATOM | 258 | CD1 | ILE | H | 34 | 49.403 | −43.596 | 7.938 | 1.00 | 24.71 | C |
| ATOM | 259 | CG2 | ILE | H | 34 | 48.067 | −44.262 | 10.488 | 1.00 | 19.38 | C |
| ATOM | 260 | C | ILE | H | 34 | 50.192 | −43.517 | 12.320 | 1.00 | 19.18 | C |
| ATOM | 261 | O | ILE | H | 34 | 50.352 | −44.412 | 13.151 | 1.00 | 19.91 | O |
| ATOM | 262 | N | GLU | H | 35 | 49.747 | −42.298 | 12.620 | 1.00 | 18.72 | N |
| ATOM | 263 | CA | GLU | H | 35 | 49.294 | −41.945 | 13.960 | 1.00 | 18.60 | C |
| ATOM | 264 | CB | GLU | H | 35 | 49.962 | −40.662 | 14.444 | 1.00 | 17.90 | C |
| ATOM | 265 | CG | GLU | H | 35 | 51.468 | −40.772 | 14.628 | 1.00 | 18.50 | C |
| ATOM | 266 | CD | GLU | H | 35 | 52.097 | −39.423 | 14.822 | 1.00 | 18.23 | C |
| ATOM | 267 | OE1 | GLU | H | 35 | 52.657 | −38.908 | 13.842 | 1.00 | 19.65 | O |
| ATOM | 268 | OE2 | GLU | H | 35 | 51.999 | −38.869 | 15.946 | 1.00 | 18.98 | O |
| ATOM | 269 | C | GLU | H | 35 | 47.816 | −41.707 | 13.966 | 1.00 | 18.82 | C |
| ATOM | 270 | O | GLU | H | 35 | 47.246 | −41.317 | 12.960 | 1.00 | 18.99 | O |
| ATOM | 271 | N | TRP | H | 36 | 47.210 | −41.920 | 15.120 | 1.00 | 19.18 | N |
| ATOM | 272 | CA | TRP | H | 36 | 45.823 | −41.581 | 15.331 | 1.00 | 19.35 | C |
| ATOM | 273 | CB | TRP | H | 36 | 45.022 | −42.812 | 15.697 | 1.00 | 19.58 | C |
| ATOM | 274 | CG | TRP | H | 36 | 44.897 | −43.757 | 14.561 | 1.00 | 19.05 | C |
| ATOM | 275 | CD1 | TRP | H | 36 | 45.828 | −44.674 | 14.158 | 1.00 | 19.73 | C |
| ATOM | 276 | NE1 | TRP | H | 36 | 45.364 | −45.370 | 13.071 | 1.00 | 21.14 | N |
| ATOM | 277 | CE2 | TRP | H | 36 | 44.116 | −44.900 | 12.743 | 1.00 | 19.80 | C |
| ATOM | 278 | CD2 | TRP | H | 36 | 43.795 | −43.875 | 13.658 | 1.00 | 20.13 | C |
| ATOM | 279 | CE3 | TRP | H | 36 | 42.566 | −43.211 | 13.528 | 1.00 | 20.19 | C |
| ATOM | 280 | CZ3 | TRP | H | 36 | 41.691 | −43.618 | 12.513 | 1.00 | 20.01 | C |
| ATOM | 281 | CH2 | TRP | H | 36 | 42.042 | −44.646 | 11.624 | 1.00 | 20.40 | C |
| ATOM | 282 | CZ2 | TRP | H | 36 | 43.246 | −45.299 | 11.728 | 1.00 | 20.10 | C |
| ATOM | 283 | C | TRP | H | 36 | 45.753 | −40.569 | 16.449 | 1.00 | 19.62 | C |
| ATOM | 284 | O | TRP | H | 36 | 46.432 | −40.711 | 17.483 | 1.00 | 18.88 | O |
| ATOM | 285 | N | ILE | H | 37 | 44.940 | −39.543 | 16.226 | 1.00 | 18.89 | N |
| ATOM | 286 | CA | ILE | H | 37 | 44.833 | −38.424 | 17.147 | 1.00 | 19.36 | C |
| ATOM | 287 | CB | ILE | H | 37 | 45.736 | −37.233 | 16.679 | 1.00 | 19.84 | C |
| ATOM | 288 | CG1 | ILE | H | 37 | 47.222 | −37.630 | 16.656 | 1.00 | 18.61 | C |
| ATOM | 289 | CD1 | ILE | H | 37 | 48.111 | −36.687 | 15.808 | 1.00 | 18.18 | C |
| ATOM | 290 | CG2 | ILE | H | 37 | 45.534 | −35.984 | 17.548 | 1.00 | 20.38 | C |
| ATOM | 291 | C | ILE | H | 37 | 43.363 | −38.010 | 17.185 | 1.00 | 19.56 | C |
| ATOM | 292 | O | ILE | H | 37 | 42.743 | −37.807 | 16.138 | 1.00 | 19.35 | O |
| ATOM | 293 | N | ARG | H | 38 | 42.787 | −37.905 | 18.383 | 1.00 | 19.51 | N |
| ATOM | 294 | CA | ARG | H | 38 | 41.410 | −37.436 | 18.455 | 1.00 | 20.31 | C |
| ATOM | 295 | CB | ARG | H | 38 | 40.498 | −38.401 | 19.224 | 1.00 | 19.40 | C |
| ATOM | 296 | CG | ARG | H | 38 | 40.752 | −38.437 | 20.725 | 1.00 | 19.89 | C |
| ATOM | 297 | CD | ARG | H | 38 | 39.874 | −39.462 | 21.430 | 1.00 | 18.63 | C |
| ATOM | 298 | NE | ARG | H | 38 | 40.008 | −39.356 | 22.884 | 1.00 | 20.10 | N |
| ATOM | 299 | CZ | ARG | H | 38 | 39.293 | −40.049 | 23.760 | 1.00 | 19.39 | C |
| ATOM | 300 | NH1 | ARG | H | 38 | 38.392 | −40.929 | 23.347 | 1.00 | 19.14 | N |
| ATOM | 301 | NH2 | ARG | H | 38 | 39.486 | −39.869 | 25.062 | 1.00 | 20.88 | N |
| ATOM | 302 | C | ARG | H | 38 | 41.316 | −36.024 | 19.022 | 1.00 | 21.38 | C |
| ATOM | 303 | O | ARG | H | 38 | 42.198 | −35.561 | 19.755 | 1.00 | 20.71 | O |
| ATOM | 304 | N | GLN | H | 39 | 40.206 | −35.370 | 18.703 | 1.00 | 23.06 | N |
| ATOM | 305 | CA | GLN | H | 39 | 39.989 | −34.004 | 19.121 | 1.00 | 25.09 | C |
| ATOM | 306 | CB | GLN | H | 39 | 40.282 | −33.029 | 17.996 | 1.00 | 24.83 | C |
| ATOM | 307 | CG | GLN | H | 39 | 40.050 | −31.565 | 18.417 | 1.00 | 24.22 | C |
| ATOM | 308 | CD | GLN | H | 39 | 40.841 | −30.614 | 17.563 | 1.00 | 21.69 | C |
| ATOM | 309 | OE1 | GLN | H | 39 | 40.767 | −30.677 | 16.353 | 1.00 | 24.10 | O |
| ATOM | 310 | NE2 | GLN | H | 39 | 41.601 | −29.720 | 18.189 | 1.00 | 22.73 | N |
| ATOM | 311 | C | GLN | H | 39 | 38.567 | −33.820 | 19.579 | 1.00 | 27.47 | C |
| ATOM | 312 | O | GLN | H | 39 | 37.613 | −33.969 | 18.808 | 1.00 | 26.57 | O |
| ATOM | 313 | N | MET | H | 40 | 38.479 | −33.471 | 20.848 | 1.00 | 30.76 | N |
| ATOM | 314 | CA | MET | H | 40 | 37.249 | −33.306 | 21.572 | 1.00 | 34.91 | C |
| ATOM | 315 | CB | MET | H | 40 | 37.560 | −33.413 | 23.079 | 1.00 | 35.30 | C |
| ATOM | 316 | CG | MET | H | 40 | 38.899 | −34.124 | 23.397 | 1.00 | 38.41 | C |
| ATOM | 317 | SD | MET | H | 40 | 38.877 | −35.928 | 23.136 | 1.00 | 42.66 | S |
| ATOM | 318 | CE | MET | H | 40 | 37.632 | −36.395 | 24.355 | 1.00 | 40.38 | C |
| ATOM | 319 | C | MET | H | 40 | 36.654 | −31.937 | 21.226 | 1.00 | 36.32 | C |
| ATOM | 320 | O | MET | H | 40 | 37.370 | −31.052 | 20.716 | 1.00 | 37.03 | O |
| ATOM | 321 | N | PRO | H | 41 | 35.341 | −31.762 | 21.480 | 1.00 | 37.77 | N |
| ATOM | 322 | CA | PRO | H | 41 | 34.592 | −30.508 | 21.313 | 1.00 | 38.36 | C |
| ATOM | 323 | CB | PRO | H | 41 | 33.570 | −30.591 | 22.457 | 1.00 | 38.72 | C |
| ATOM | 324 | CG | PRO | H | 41 | 33.297 | −32.098 | 22.602 | 1.00 | 38.75 | C |
| ATOM | 325 | CD | PRO | H | 41 | 34.440 | −32.846 | 21.920 | 1.00 | 38.39 | C |
| ATOM | 326 | C | PRO | H | 41 | 35.418 | −29.209 | 21.427 | 1.00 | 38.57 | C |
| ATOM | 327 | O | PRO | H | 41 | 35.715 | −28.761 | 22.551 | 1.00 | 39.78 | O |
| ATOM | 328 | N | GLY | H | 42 | 35.779 | −28.623 | 20.277 | 1.00 | 38.23 | N |
| ATOM | 329 | CA | GLY | H | 42 | 36.632 | −27.410 | 20.214 | 1.00 | 37.80 | C |
| ATOM | 330 | C | GLY | H | 42 | 37.730 | −27.384 | 21.272 | 1.00 | 37.39 | C |
| ATOM | 331 | O | GLY | H | 42 | 37.955 | −26.369 | 21.966 | 1.00 | 37.25 | O |
| ATOM | 332 | N | GLN | H | 43 | 38.425 | −28.511 | 21.380 | 1.00 | 35.83 | N |
| ATOM | 333 | CA | GLN | H | 43 | 39.287 | −28.775 | 22.506 | 1.00 | 34.40 | C |
| ATOM | 334 | CB | GLN | H | 43 | 38.635 | −29.866 | 23.358 | 1.00 | 35.25 | C |
| ATOM | 335 | CG | GLN | H | 43 | 39.019 | −29.909 | 24.814 | 1.00 | 38.46 | C |
| ATOM | 336 | CD | GLN | H | 43 | 37.878 | −30.439 | 25.674 | 1.00 | 42.19 | C |
| ATOM | 337 | OE1 | GLN | H | 43 | 36.783 | −30.743 | 25.168 | 1.00 | 43.67 | O |
| ATOM | 338 | NE2 | GLN | H | 43 | 38.125 | −30.552 | 26.977 | 1.00 | 42.61 | N |
| ATOM | 339 | C | GLN | H | 43 | 40.668 | −29.218 | 22.019 | 1.00 | 32.36 | C |
| ATOM | 340 | O | GLN | H | 43 | 41.041 | −28.984 | 20.859 | 1.00 | 31.29 | O |
| ATOM | 341 | N | GLY | H | 44 | 41.409 | −29.861 | 22.917 | 1.00 | 30.09 | N |
| ATOM | 342 | CA | GLY | H | 44 | 42.773 | −30.261 | 22.659 | 1.00 | 28.65 | C |
| ATOM | 343 | C | GLY | H | 44 | 42.905 | −31.529 | 21.835 | 1.00 | 27.03 | C |
| ATOM | 344 | O | GLY | H | 44 | 41.913 | −32.225 | 21.553 | 1.00 | 26.74 | O |
| ATOM | 345 | N | LEU | H | 45 | 44.146 | −31.816 | 21.454 | 1.00 | 25.72 | N |
| ATOM | 346 | CA | LEU | H | 45 | 44.473 | −32.983 | 20.650 | 1.00 | 24.67 | C |
| ATOM | 347 | CB | LEU | H | 45 | 45.582 | −32.651 | 19.647 | 1.00 | 24.45 | C |
| ATOM | 348 | CG | LEU | H | 45 | 45.248 | −31.544 | 18.639 | 1.00 | 24.63 | C |
| ATOM | 349 | CD1 | LEU | H | 45 | 46.493 | −31.077 | 17.897 | 1.00 | 23.84 | C |
| ATOM | 350 | CD2 | LEU | H | 45 | 44.202 | −32.019 | 17.632 | 1.00 | 24.64 | C |
| ATOM | 351 | C | LEU | H | 45 | 44.925 | −34.054 | 21.616 | 1.00 | 24.16 | C |
| ATOM | 352 | O | LEU | H | 45 | 45.759 | −33.786 | 22.495 | 1.00 | 24.84 | O |
| ATOM | 353 | N | GLU | H | 46 | 44.342 | −35.242 | 21.482 | 1.00 | 22.21 | N |
| ATOM | 354 | CA | GLU | H | 46 | 44.707 | −36.375 | 22.311 | 1.00 | 21.34 | C |
| ATOM | 355 | CB | GLU | H | 46 | 43.493 | −36.944 | 23.047 | 1.00 | 21.72 | C |
| ATOM | 356 | CG | GLU | H | 46 | 42.853 | −35.982 | 24.082 | 1.00 | 22.79 | C |
| ATOM | 357 | CD | GLU | H | 46 | 41.821 | −36.664 | 24.995 | 1.00 | 27.09 | C |
| ATOM | 358 | OE1 | GLU | H | 46 | 41.112 | −37.588 | 24.545 | 1.00 | 27.61 | O |
| ATOM | 359 | OE2 | GLU | H | 46 | 41.705 | −36.258 | 26.171 | 1.00 | 27.92 | O |
| ATOM | 360 | C | GLU | H | 46 | 45.341 | −37.406 | 21.388 | 1.00 | 20.45 | C |
| ATOM | 361 | O | GLU | H | 46 | 44.705 | −37.881 | 20.444 | 1.00 | 19.79 | O |
| ATOM | 362 | N | TRP | H | 47 | 46.616 | −37.683 | 21.625 | 1.00 | 19.62 | N |
| ATOM | 363 | CA | TRP | H | 47 | 47.349 | −38.670 | 20.843 | 1.00 | 18.99 | C |
| ATOM | 364 | CB | TRP | H | 47 | 48.841 | −38.382 | 20.935 | 1.00 | 19.25 | C |
| ATOM | 365 | CG | TRP | H | 47 | 49.760 | −39.173 | 20.020 | 1.00 | 17.86 | C |
| ATOM | 366 | CD1 | TRP | H | 47 | 50.109 | −38.864 | 18.736 | 1.00 | 18.59 | C |
| ATOM | 367 | NE1 | TRP | H | 47 | 51.010 | −39.799 | 18.250 | 1.00 | 16.61 | N |
| ATOM | 368 | CE2 | TRP | H | 47 | 51.255 | −40.728 | 19.227 | 1.00 | 17.46 | C |
| ATOM | 369 | CD2 | TRP | H | 47 | 50.498 | −40.357 | 20.364 | 1.00 | 18.24 | C |
| ATOM | 370 | CE3 | TRP | H | 47 | 50.585 | −41.146 | 21.519 | 1.00 | 18.36 | C |
| ATOM | 371 | CZ3 | TRP | H | 47 | 51.427 | −42.276 | 21.503 | 1.00 | 19.85 | C |
| ATOM | 372 | CH2 | TRP | H | 47 | 52.168 | −42.614 | 20.363 | 1.00 | 19.10 | C |
| ATOM | 373 | CZ2 | TRP | H | 47 | 52.102 | −41.855 | 19.214 | 1.00 | 19.70 | C |
| ATOM | 374 | C | TRP | H | 47 | 47.013 | −40.092 | 21.318 | 1.00 | 18.96 | C |
| ATOM | 375 | O | TRP | H | 47 | 47.111 | −40.422 | 22.517 | 1.00 | 18.54 | O |
| ATOM | 376 | N | ILE | H | 48 | 46.602 | −40.930 | 20.367 | 1.00 | 17.96 | N |
| ATOM | 377 | CA | ILE | H | 48 | 46.189 | −42.307 | 20.668 | 1.00 | 17.37 | C |
| ATOM | 378 | CB | ILE | H | 48 | 44.942 | −42.714 | 19.808 | 1.00 | 17.56 | C |
| ATOM | 379 | CG1 | ILE | H | 48 | 43.757 | −41.781 | 20.130 | 1.00 | 17.69 | C |
| ATOM | 380 | CD1 | ILE | H | 48 | 42.682 | −41.752 | 19.003 | 1.00 | 17.02 | C |
| ATOM | 381 | CG2 | ILE | H | 48 | 44.523 | −44.193 | 20.053 | 1.00 | 16.95 | C |
| ATOM | 382 | C | ILE | H | 48 | 47.344 | −43.294 | 20.475 | 1.00 | 17.34 | C |
| ATOM | 383 | O | ILE | H | 48 | 47.604 | −44.146 | 21.335 | 1.00 | 18.13 | O |
| ATOM | 384 | N | GLY | H | 49 | 48.017 | −43.213 | 19.332 | 1.00 | 17.42 | N |
| ATOM | 385 | CA | GLY | H | 49 | 49.142 | −44.113 | 19.084 | 1.00 | 18.02 | C |
| ATOM | 386 | C | GLY | H | 49 | 49.722 | −44.048 | 17.689 | 1.00 | 18.28 | C |
| ATOM | 387 | O | GLY | H | 49 | 49.265 | −43.272 | 16.838 | 1.00 | 19.61 | O |
| ATOM | 388 | N | LEU | H | 50 | 50.708 | −44.898 | 17.440 | 1.00 | 17.61 | N |
| ATOM | 389 | CA | LEU | H | 50 | 51.410 | −44.902 | 16.171 | 1.00 | 17.63 | C |
| ATOM | 390 | CB | LEU | H | 50 | 52.745 | −44.148 | 16.276 | 1.00 | 16.69 | C |
| ATOM | 391 | CG | LEU | H | 50 | 53.769 | −44.189 | 15.119 | 1.00 | 17.69 | C |
| ATOM | 392 | CD1 | LEU | H | 50 | 54.683 | −42.961 | 15.119 | 1.00 | 15.94 | C |
| ATOM | 393 | CD2 | LEU | H | 50 | 54.618 | −45.477 | 15.131 | 1.00 | 16.84 | C |
| ATOM | 394 | C | LEU | H | 50 | 51.605 | −46.343 | 15.763 | 1.00 | 17.91 | C |
| ATOM | 395 | O | LEU | H | 50 | 51.791 | −47.235 | 16.617 | 1.00 | 16.96 | O |
| ATOM | 396 | N | ILE | H | 51 | 51.523 | −46.579 | 14.459 | 1.00 | 17.72 | N |
| ATOM | 397 | CA | ILE | H | 51 | 51.737 | −47.905 | 13.929 | 1.00 | 18.04 | C |
| ATOM | 398 | CB | ILE | H | 51 | 50.415 | −48.649 | 13.631 | 1.00 | 17.66 | C |
| ATOM | 399 | CG1 | ILE | H | 51 | 50.708 | −50.056 | 13.104 | 1.00 | 16.66 | C |
| ATOM | 400 | CD1 | ILE | H | 51 | 49.485 | −50.984 | 13.088 | 1.00 | 14.02 | C |
| ATOM | 401 | CG2 | ILE | H | 51 | 49.553 | −47.869 | 12.637 | 1.00 | 17.76 | C |
| ATOM | 402 | C | ILE | H | 51 | 52.589 | −47.842 | 12.662 | 1.00 | 18.82 | C |
| ATOM | 403 | O | ILE | H | 51 | 52.421 | −46.957 | 11.833 | 1.00 | 19.08 | O |
| ATOM | 404 | N | ASN | H | 52 | 53.500 | −48.801 | 12.548 | 1.00 | 19.47 | N |
| ATOM | 405 | CA | ASN | H | 52 | 54.277 | −49.020 | 11.339 | 1.00 | 20.44 | C |
| ATOM | 406 | CB | ASN | H | 52 | 55.771 | −49.101 | 11.698 | 1.00 | 20.51 | C |
| ATOM | 407 | CG | ASN | H | 52 | 56.630 | −49.555 | 10.529 | 1.00 | 23.85 | C |
| ATOM | 408 | OD1 | ASN | H | 52 | 56.588 | −48.962 | 9.434 | 1.00 | 23.81 | O |
| ATOM | 409 | ND2 | ASN | H | 52 | 57.413 | −50.614 | 10.752 | 1.00 | 24.36 | N |
| ATOM | 410 | C | ASN | H | 52 | 53.780 | −50.312 | 10.675 | 1.00 | 19.86 | C |
| ATOM | 411 | O | ASN | H | 52 | 54.070 | −51.408 | 11.165 | 1.00 | 20.64 | O |
| ATOM | 412 | N | PRO | H | 52A | 53.039 | −50.201 | 9.551 | 1.00 | 19.69 | N |
| ATOM | 413 | CA | PRO | H | 52A | 52.442 | −51.414 | 8.979 | 1.00 | 19.75 | C |
| ATOM | 414 | CB | PRO | H | 52A | 51.651 | −50.886 | 7.774 | 1.00 | 20.02 | C |
| ATOM | 415 | CG | PRO | H | 52A | 51.405 | −49.433 | 8.088 | 1.00 | 19.40 | C |
| ATOM | 416 | CD | PRO | H | 52A | 52.649 | −48.998 | 8.790 | 1.00 | 19.71 | C |
| ATOM | 417 | C | PRO | H | 52A | 53.440 | −52.476 | 8.530 | 1.00 | 20.13 | C |
| ATOM | 418 | O | PRO | H | 52A | 53.075 | −53.647 | 8.457 | 1.00 | 20.54 | O |
| ATOM | 419 | N | GLY | H | 53 | 54.682 | −52.083 | 8.251 | 1.00 | 20.26 | N |
| ATOM | 420 | CA | GLY | H | 53 | 55.686 | −53.023 | 7.755 | 1.00 | 21.34 | C |
| ATOM | 421 | C | GLY | H | 53 | 56.286 | −53.960 | 8.801 | 1.00 | 21.48 | C |
| ATOM | 422 | O | GLY | H | 53 | 57.053 | −54.860 | 8.454 | 1.00 | 21.28 | O |
| ATOM | 423 | N | SER | H | 54 | 55.970 | −53.729 | 10.076 | 1.00 | 21.19 | N |
| ATOM | 424 | CA | SER | H | 54 | 56.496 | −54.547 | 11.177 | 1.00 | 21.14 | C |
| ATOM | 425 | CB | SER | H | 54 | 57.742 | −53.887 | 11.771 | 1.00 | 20.84 | C |
| ATOM | 426 | OG | SER | H | 54 | 57.375 | −52.715 | 12.504 | 1.00 | 21.65 | O |
| ATOM | 427 | C | SER | H | 54 | 55.421 | −54.707 | 12.258 | 1.00 | 21.10 | C |
| ATOM | 428 | O | SER | H | 54 | 54.259 | −54.334 | 12.043 | 1.00 | 20.55 | O |
| ATOM | 429 | N | ASP | H | 55 | 55.806 | −55.257 | 13.411 | 1.00 | 20.62 | N |
| ATOM | 430 | CA | ASP | H | 55 | 54.905 | −55.325 | 14.561 | 1.00 | 21.20 | C |
| ATOM | 431 | CB | ASP | H | 55 | 55.278 | −56.472 | 15.505 | 1.00 | 21.24 | C |
| ATOM | 432 | CG | ASP | H | 55 | 55.055 | −57.845 | 14.908 | 1.00 | 21.67 | C |
| ATOM | 433 | OD1 | ASP | H | 55 | 55.774 | −58.765 | 15.338 | 1.00 | 23.35 | O |
| ATOM | 434 | OD2 | ASP | H | 55 | 54.190 | −58.027 | 14.028 | 1.00 | 21.61 | O |
| ATOM | 435 | C | ASP | H | 55 | 54.951 | −54.026 | 15.376 | 1.00 | 21.27 | C |
| ATOM | 436 | O | ASP | H | 55 | 54.304 | −53.947 | 16.422 | 1.00 | 21.25 | O |
| ATOM | 437 | N | TYR | H | 56 | 55.717 | −53.026 | 14.932 | 1.00 | 21.07 | N |
| ATOM | 438 | CA | TYR | H | 56 | 55.883 | −51.811 | 15.748 | 1.00 | 21.75 | C |
| ATOM | 439 | CB | TYR | H | 56 | 56.979 | −50.867 | 15.244 | 1.00 | 21.84 | C |
| ATOM | 440 | CG | TYR | H | 56 | 57.388 | −49.877 | 16.330 | 1.00 | 24.82 | C |
| ATOM | 441 | CD1 | TYR | H | 56 | 58.438 | −50.167 | 17.211 | 1.00 | 26.84 | C |
| ATOM | 442 | CE1 | TYR | H | 56 | 58.793 | −49.273 | 18.228 | 1.00 | 27.64 | C |
| ATOM | 443 | CZ | TYR | H | 56 | 58.074 | −48.097 | 18.365 | 1.00 | 29.10 | C |
| ATOM | 444 | OH | TYR | H | 56 | 58.391 | −47.210 | 19.353 | 1.00 | 32.22 | O |
| ATOM | 445 | CE2 | TYR | H | 56 | 57.024 | −47.798 | 17.516 | 1.00 | 27.29 | C |
| ATOM | 446 | CD2 | TYR | H | 56 | 56.680 | −48.685 | 16.518 | 1.00 | 25.48 | C |
| ATOM | 447 | C | TYR | H | 56 | 54.574 | −51.038 | 15.952 | 1.00 | 21.40 | C |
| ATOM | 448 | O | TYR | H | 56 | 53.893 | −50.657 | 14.990 | 1.00 | 20.84 | O |
| ATOM | 449 | N | THR | H | 57 | 54.220 | −50.868 | 17.222 | 1.00 | 21.30 | N |
| ATOM | 450 | CA | THR | H | 57 | 53.140 | −49.975 | 17.621 | 1.00 | 21.03 | C |
| ATOM | 451 | CB | THR | H | 57 | 51.789 | −50.723 | 17.833 | 1.00 | 21.24 | C |
| ATOM | 452 | OG1 | THR | H | 57 | 51.908 | −51.650 | 18.915 | 1.00 | 21.05 | O |
| ATOM | 453 | CG2 | THR | H | 57 | 51.324 | −51.480 | 16.583 | 1.00 | 21.17 | C |
| ATOM | 454 | C | THR | H | 57 | 53.555 | −49.302 | 18.922 | 1.00 | 21.47 | C |
| ATOM | 455 | O | THR | H | 57 | 54.373 | −49.843 | 19.681 | 1.00 | 21.36 | O |
| ATOM | 456 | N | ASN | H | 58 | 53.017 | −48.111 | 19.173 | 1.00 | 20.85 | N |
| ATOM | 457 | CA | ASN | H | 58 | 53.128 | −47.495 | 20.483 | 1.00 | 21.00 | C |
| ATOM | 458 | CB | ASN | H | 58 | 54.272 | −46.476 | 20.540 | 1.00 | 20.69 | C |
| ATOM | 459 | CG | ASN | H | 58 | 54.498 | −45.943 | 21.950 | 1.00 | 22.11 | C |
| ATOM | 460 | OD1 | ASN | H | 58 | 53.802 | −45.031 | 22.397 | 1.00 | 23.55 | O |
| ATOM | 461 | ND2 | ASN | H | 58 | 55.466 | −46.517 | 22.658 | 1.00 | 23.08 | N |
| ATOM | 462 | C | ASN | H | 58 | 51.806 | −46.832 | 20.809 | 1.00 | 20.84 | C |
| ATOM | 463 | O | ASN | H | 58 | 51.244 | −46.133 | 19.963 | 1.00 | 20.83 | O |
| ATOM | 464 | N | TYR | H | 59 | 51.313 | −47.073 | 22.018 | 1.00 | 20.31 | N |
| ATOM | 465 | CA | TYR | H | 59 | 49.983 | −46.636 | 22.428 | 1.00 | 20.36 | C |
| ATOM | 466 | CB | TYR | H | 59 | 49.145 | −47.817 | 22.940 | 1.00 | 20.66 | C |
| ATOM | 467 | CG | TYR | H | 59 | 48.687 | −48.820 | 21.909 | 1.00 | 21.84 | C |
| ATOM | 468 | CD1 | TYR | H | 59 | 49.429 | −49.966 | 21.648 | 1.00 | 20.75 | C |
| ATOM | 469 | CE1 | TYR | H | 59 | 48.989 | −50.916 | 20.696 | 1.00 | 23.72 | C |
| ATOM | 470 | CZ | TYR | H | 59 | 47.804 | −50.679 | 20.010 | 1.00 | 22.22 | C |
| ATOM | 471 | OH | TYR | H | 59 | 47.344 | −51.579 | 19.084 | 1.00 | 18.65 | O |
| ATOM | 472 | CE2 | TYR | H | 59 | 47.054 | −49.550 | 20.279 | 1.00 | 22.69 | C |
| ATOM | 473 | CD2 | TYR | H | 59 | 47.489 | −48.637 | 21.226 | 1.00 | 22.56 | C |
| ATOM | 474 | C | TYR | H | 59 | 50.090 | −45.687 | 23.585 | 1.00 | 20.09 | C |
| ATOM | 475 | O | TYR | H | 59 | 50.922 | −45.879 | 24.475 | 1.00 | 19.51 | O |
| ATOM | 476 | N | ASN | H | 60 | 49.221 | −44.692 | 23.592 | 1.00 | 19.58 | N |
| ATOM | 477 | CA | ASN | H | 60 | 48.993 | −43.898 | 24.786 | 1.00 | 20.27 | C |
| ATOM | 478 | CB | ASN | H | 60 | 48.050 | −42.737 | 24.449 | 1.00 | 19.95 | C |
| ATOM | 479 | CG | ASN | H | 60 | 47.788 | −41.816 | 25.628 | 1.00 | 19.99 | C |
| ATOM | 480 | OD1 | ASN | H | 60 | 47.989 | −42.186 | 26.791 | 1.00 | 18.43 | O |
| ATOM | 481 | ND2 | ASN | H | 60 | 47.317 | −40.608 | 25.330 | 1.00 | 16.13 | N |
| ATOM | 482 | C | ASN | H | 60 | 48.408 | −44.827 | 25.860 | 1.00 | 20.55 | C |
| ATOM | 483 | O | ASN | H | 60 | 47.332 | −45.403 | 25.663 | 1.00 | 19.20 | O |
| ATOM | 484 | N | GLU | H | 61 | 49.146 | −44.990 | 26.965 | 1.00 | 21.83 | N |
| ATOM | 485 | CA | GLU | H | 61 | 48.753 | −45.867 | 28.089 | 1.00 | 23.95 | C |
| ATOM | 486 | CB | GLU | H | 61 | 49.768 | −45.789 | 29.265 | 1.00 | 24.54 | C |
| ATOM | 487 | CG | GLU | H | 61 | 49.246 | −46.378 | 30.614 | 1.00 | 27.32 | C |
| ATOM | 488 | CD | GLU | H | 61 | 50.344 | −46.857 | 31.584 | 1.00 | 32.41 | C |
| ATOM | 489 | OE1 | GLU | H | 61 | 51.559 | −46.691 | 31.298 | 1.00 | 33.34 | O |
| ATOM | 490 | OE2 | GLU | H | 61 | 49.972 | −47.421 | 32.647 | 1.00 | 33.85 | O |
| ATOM | 491 | C | GLU | H | 61 | 47.323 | −45.630 | 28.582 | 1.00 | 24.53 | C |
| ATOM | 492 | O | GLU | H | 61 | 46.636 | −46.571 | 28.995 | 1.00 | 24.85 | O |
| ATOM | 493 | N | ASN | H | 62 | 46.881 | −44.378 | 28.528 | 1.00 | 25.18 | N |
| ATOM | 494 | CA | ASN | H | 62 | 45.538 | −44.006 | 28.947 | 1.00 | 26.22 | C |
| ATOM | 495 | CB | ASN | H | 62 | 45.374 | −42.490 | 28.896 | 1.00 | 26.92 | C |
| ATOM | 496 | CG | ASN | H | 62 | 46.316 | −41.775 | 29.844 | 1.00 | 28.86 | C |
| ATOM | 497 | OD1 | ASN | H | 62 | 46.442 | −42.143 | 31.017 | 1.00 | 31.76 | O |
| ATOM | 498 | ND2 | ASN | H | 62 | 46.992 | −40.752 | 29.337 | 1.00 | 30.06 | N |
| ATOM | 499 | C | ASN | H | 62 | 44.419 | −44.661 | 28.128 | 1.00 | 26.64 | C |
| ATOM | 500 | O | ASN | H | 62 | 43.282 | −44.756 | 28.605 | 1.00 | 25.87 | O |
| ATOM | 501 | N | PHE | H | 63 | 44.747 | −45.086 | 26.907 | 1.00 | 26.38 | N |
| ATOM | 502 | CA | PHE | H | 63 | 43.795 | −45.754 | 26.016 | 1.00 | 27.05 | C |
| ATOM | 503 | CB | PHE | H | 63 | 43.665 | −45.001 | 24.694 | 1.00 | 26.53 | C |
| ATOM | 504 | CG | PHE | H | 63 | 43.370 | −43.537 | 24.842 | 1.00 | 27.42 | C |
| ATOM | 505 | CD1 | PHE | H | 63 | 44.144 | −42.597 | 24.165 | 1.00 | 26.84 | C |
| ATOM | 506 | CE1 | PHE | H | 63 | 43.880 | −41.238 | 24.277 | 1.00 | 27.63 | C |
| ATOM | 507 | CZ | PHE | H | 63 | 42.837 | −40.803 | 25.078 | 1.00 | 28.71 | C |
| ATOM | 508 | CE2 | PHE | H | 63 | 42.053 | −41.733 | 25.766 | 1.00 | 29.35 | C |
| ATOM | 509 | CD2 | PHE | H | 63 | 42.323 | −43.096 | 25.639 | 1.00 | 26.99 | C |
| ATOM | 510 | C | PHE | H | 63 | 44.153 | −47.214 | 25.691 | 1.00 | 27.52 | C |
| ATOM | 511 | O | PHE | H | 63 | 43.497 | −47.834 | 24.840 | 1.00 | 27.64 | O |
| ATOM | 512 | N | LYS | H | 64 | 45.184 | −47.765 | 26.334 | 1.00 | 28.02 | N |
| ATOM | 513 | CA | LYS | H | 64 | 45.561 | −49.161 | 26.057 | 1.00 | 28.57 | C |
| ATOM | 514 | CB | LYS | H | 64 | 46.922 | −49.537 | 26.675 | 1.00 | 28.93 | C |
| ATOM | 515 | CG | LYS | H | 64 | 46.893 | −49.763 | 28.177 | 1.00 | 30.28 | C |
| ATOM | 516 | CD | LYS | H | 64 | 48.147 | −50.468 | 28.667 | 1.00 | 33.79 | C |
| ATOM | 517 | CE | LYS | H | 64 | 47.914 | −51.032 | 30.051 | 1.00 | 33.88 | C |
| ATOM | 518 | NZ | LYS | H | 64 | 48.958 | −52.035 | 30.413 | 1.00 | 36.40 | N |
| ATOM | 519 | C | LYS | H | 64 | 44.441 | −50.098 | 26.518 | 1.00 | 28.03 | C |
| ATOM | 520 | O | LYS | H | 64 | 43.907 | −49.960 | 27.625 | 1.00 | 28.61 | O |
| ATOM | 521 | N | GLY | H | 65 | 44.042 | −51.023 | 25.661 | 1.00 | 27.62 | N |
| ATOM | 522 | CA | GLY | H | 65 | 42.918 | −51.917 | 26.029 | 1.00 | 27.38 | C |
| ATOM | 523 | C | GLY | H | 65 | 41.519 | −51.337 | 25.830 | 1.00 | 26.25 | C |
| ATOM | 524 | O | GLY | H | 65 | 40.529 | −51.991 | 26.132 | 1.00 | 25.77 | O |
| ATOM | 525 | N | GLN | H | 66 | 41.441 | −50.088 | 25.375 | 1.00 | 25.40 | N |
| ATOM | 526 | CA | GLN | H | 66 | 40.284 | −49.646 | 24.608 | 1.00 | 24.58 | C |
| ATOM | 527 | CB | GLN | H | 66 | 39.791 | −48.254 | 25.024 | 1.00 | 25.18 | C |
| ATOM | 528 | CG | GLN | H | 66 | 38.935 | −48.235 | 26.291 | 1.00 | 28.33 | C |
| ATOM | 529 | CD | GLN | H | 66 | 39.770 | −48.160 | 27.559 | 1.00 | 32.57 | C |
| ATOM | 530 | OE1 | GLN | H | 66 | 40.585 | −47.243 | 27.733 | 1.00 | 34.98 | O |
| ATOM | 531 | NE2 | GLN | H | 66 | 39.573 | −49.129 | 28.457 | 1.00 | 34.28 | N |
| ATOM | 532 | C | GLN | H | 66 | 40.705 | −49.634 | 23.148 | 1.00 | 23.07 | C |
| ATOM | 533 | O | GLN | H | 66 | 39.993 | −50.135 | 22.293 | 1.00 | 22.67 | O |
| ATOM | 534 | N | ALA | H | 67 | 41.883 | −49.077 | 22.875 | 1.00 | 22.03 | N |
| ATOM | 535 | CA | ALA | H | 67 | 42.346 | −48.859 | 21.502 | 1.00 | 21.08 | C |
| ATOM | 536 | CB | ALA | H | 67 | 43.139 | −47.569 | 21.415 | 1.00 | 21.07 | C |
| ATOM | 537 | C | ALA | H | 67 | 43.165 | −50.027 | 20.972 | 1.00 | 20.88 | C |
| ATOM | 538 | O | ALA | H | 67 | 43.965 | −50.614 | 21.708 | 1.00 | 20.82 | O |
| ATOM | 539 | N | THR | H | 68 | 42.951 | −50.365 | 19.702 | 1.00 | 19.96 | N |
| ATOM | 540 | CA | THR | H | 68 | 43.767 | −51.357 | 18.999 | 1.00 | 20.03 | C |
| ATOM | 541 | CB | THR | H | 68 | 43.064 | −52.721 | 18.863 | 1.00 | 20.00 | C |
| ATOM | 542 | OG1 | THR | H | 68 | 42.716 | −53.231 | 20.158 | 1.00 | 20.77 | O |
| ATOM | 543 | CG2 | THR | H | 68 | 43.952 | −53.734 | 18.162 | 1.00 | 20.49 | C |
| ATOM | 544 | C | THR | H | 68 | 44.060 | −50.793 | 17.599 | 1.00 | 19.97 | C |
| ATOM | 545 | O | THR | H | 68 | 43.132 | −50.506 | 16.829 | 1.00 | 20.17 | O |
| ATOM | 546 | N | LEU | H | 69 | 45.339 | −50.631 | 17.286 | 1.00 | 19.54 | N |
| ATOM | 547 | CA | LEU | H | 69 | 45.766 | −50.185 | 15.960 | 1.00 | 19.51 | C |
| ATOM | 548 | CB | LEU | H | 69 | 46.927 | −49.199 | 16.053 | 1.00 | 20.03 | C |
| ATOM | 549 | CG | LEU | H | 69 | 46.777 | −47.834 | 16.749 | 1.00 | 21.26 | C |
| ATOM | 550 | CD1 | LEU | H | 69 | 47.940 | −46.917 | 16.388 | 1.00 | 21.85 | C |
| ATOM | 551 | CD2 | LEU | H | 69 | 45.492 | −47.165 | 16.396 | 1.00 | 22.82 | C |
| ATOM | 552 | C | LEU | H | 69 | 46.185 | −51.397 | 15.146 | 1.00 | 19.65 | C |
| ATOM | 553 | O | LEU | H | 69 | 46.879 | −52.278 | 15.650 | 1.00 | 18.93 | O |
| ATOM | 554 | N | SER | H | 70 | 45.758 | −51.433 | 13.888 | 1.00 | 19.19 | N |
| ATOM | 555 | CA | SER | H | 70 | 46.114 | −52.510 | 12.980 | 1.00 | 19.72 | C |
| ATOM | 556 | CB | SER | H | 70 | 45.045 | −53.615 | 13.019 | 1.00 | 19.46 | C |
| ATOM | 557 | OG | SER | H | 70 | 43.778 | −53.099 | 12.619 | 1.00 | 20.91 | O |
| ATOM | 558 | C | SER | H | 70 | 46.259 | −51.975 | 11.561 | 1.00 | 19.49 | C |
| ATOM | 559 | O | SER | H | 70 | 45.824 | −50.868 | 11.259 | 1.00 | 19.31 | O |
| ATOM | 560 | N | ALA | H | 71 | 46.845 | −52.773 | 10.680 | 1.00 | 19.93 | N |
| ATOM | 561 | CA | ALA | H | 71 | 47.007 | −52.358 | 9.300 | 1.00 | 20.95 | C |
| ATOM | 562 | CB | ALA | H | 71 | 48.307 | −51.534 | 9.127 | 1.00 | 21.03 | C |
| ATOM | 563 | C | ALA | H | 71 | 46.973 | −53.527 | 8.344 | 1.00 | 21.63 | C |
| ATOM | 564 | O | ALA | H | 71 | 47.483 | −54.612 | 8.641 | 1.00 | 21.53 | O |
| ATOM | 565 | N | ASP | H | 72 | 46.340 | −53.301 | 7.199 | 1.00 | 22.47 | N |
| ATOM | 566 | CA | ASP | H | 72 | 46.244 | −54.304 | 6.147 | 1.00 | 23.22 | C |
| ATOM | 567 | CB | ASP | H | 72 | 44.778 | −54.481 | 5.724 | 1.00 | 23.41 | C |
| ATOM | 568 | CG | ASP | H | 72 | 44.578 | −55.579 | 4.674 | 1.00 | 25.15 | C |
| ATOM | 569 | OD1 | ASP | H | 72 | 45.569 | −56.165 | 4.186 | 1.00 | 27.39 | O |
| ATOM | 570 | OD2 | ASP | H | 72 | 43.405 | −55.853 | 4.337 | 1.00 | 27.20 | O |
| ATOM | 571 | C | ASP | H | 72 | 47.131 | −53.847 | 4.987 | 1.00 | 23.72 | C |
| ATOM | 572 | O | ASP | H | 72 | 46.786 | −52.924 | 4.243 | 1.00 | 23.15 | O |
| ATOM | 573 | N | LYS | H | 73 | 48.285 | −54.496 | 4.859 | 1.00 | 24.32 | N |
| ATOM | 574 | CA | LYS | H | 73 | 49.273 | −54.170 | 3.827 | 1.00 | 25.15 | C |
| ATOM | 575 | CB | LYS | H | 73 | 50.575 | −54.948 | 4.064 | 1.00 | 25.47 | C |
| ATOM | 576 | CG | LYS | H | 73 | 51.134 | −54.812 | 5.493 | 1.00 | 26.41 | C |
| ATOM | 577 | CD | LYS | H | 73 | 52.495 | −55.520 | 5.653 | 1.00 | 28.61 | C |
| ATOM | 578 | CE | LYS | H | 73 | 52.354 | −57.040 | 5.696 | 1.00 | 29.16 | C |
| ATOM | 579 | NZ | LYS | H | 73 | 51.680 | −57.476 | 6.952 | 1.00 | 29.19 | N |
| ATOM | 580 | C | LYS | H | 73 | 48.757 | −54.442 | 2.412 | 1.00 | 25.39 | C |
| ATOM | 581 | O | LYS | H | 73 | 49.151 | −53.755 | 1.469 | 1.00 | 26.06 | O |
| ATOM | 582 | N | SER | H | 74 | 47.879 | −55.431 | 2.260 | 1.00 | 25.05 | N |
| ATOM | 583 | CA | SER | H | 74 | 47.372 | −55.777 | 0.931 | 1.00 | 24.93 | C |
| ATOM | 584 | CB | SER | H | 74 | 46.754 | −57.189 | 0.903 | 1.00 | 24.90 | C |
| ATOM | 585 | OG | SER | H | 74 | 45.546 | −57.253 | 1.651 | 1.00 | 25.91 | O |
| ATOM | 586 | C | SER | H | 74 | 46.406 | −54.716 | 0.386 | 1.00 | 24.44 | C |
| ATOM | 587 | O | SER | H | 74 | 46.261 | −54.572 | −0.826 | 1.00 | 24.48 | O |
| ATOM | 588 | N | SER | H | 75 | 45.763 | −53.968 | 1.282 | 1.00 | 23.97 | N |
| ATOM | 589 | CA | SER | H | 75 | 44.830 | −52.904 | 0.884 | 1.00 | 23.30 | C |
| ATOM | 590 | CB | SER | H | 75 | 43.438 | −53.139 | 1.497 | 1.00 | 23.44 | C |
| ATOM | 591 | OG | SER | H | 75 | 43.416 | −52.811 | 2.882 | 1.00 | 24.38 | O |
| ATOM | 592 | C | SER | H | 75 | 45.307 | −51.485 | 1.222 | 1.00 | 22.42 | C |
| ATOM | 593 | O | SER | H | 75 | 44.602 | −50.519 | 0.937 | 1.00 | 22.11 | O |
| ATOM | 594 | N | SER | H | 76 | 46.494 | −51.361 | 1.820 | 1.00 | 21.25 | N |
| ATOM | 595 | CA | SER | H | 76 | 47.013 | −50.064 | 2.281 | 1.00 | 20.08 | C |
| ATOM | 596 | CB | SER | H | 76 | 47.405 | −49.165 | 1.094 | 1.00 | 19.96 | C |
| ATOM | 597 | OG | SER | H | 76 | 48.381 | −49.797 | 0.292 | 1.00 | 20.18 | O |
| ATOM | 598 | C | SER | H | 76 | 46.018 | −49.332 | 3.187 | 1.00 | 19.72 | C |
| ATOM | 599 | O | SER | H | 76 | 45.804 | −48.121 | 3.064 | 1.00 | 19.00 | O |
| ATOM | 600 | N | THR | H | 77 | 45.422 | −50.067 | 4.113 | 1.00 | 18.86 | N |
| ATOM | 601 | CA | THR | H | 77 | 44.462 | −49.465 | 5.020 | 1.00 | 18.68 | C |
| ATOM | 602 | CB | THR | H | 77 | 43.080 | −50.096 | 4.801 | 1.00 | 18.43 | C |
| ATOM | 603 | OG1 | THR | H | 77 | 42.835 | −50.208 | 3.391 | 1.00 | 19.82 | O |
| ATOM | 604 | CG2 | THR | H | 77 | 42.005 | −49.279 | 5.427 | 1.00 | 18.93 | C |
| ATOM | 605 | C | THR | H | 77 | 44.895 | −49.646 | 6.477 | 1.00 | 18.59 | C |
| ATOM | 606 | O | THR | H | 77 | 45.224 | −50.767 | 6.899 | 1.00 | 18.17 | O |
| ATOM | 607 | N | ALA | H | 78 | 44.903 | −48.549 | 7.230 | 1.00 | 18.05 | N |
| ATOM | 608 | CA | ALA | H | 78 | 45.089 | −48.631 | 8.676 | 1.00 | 18.67 | C |
| ATOM | 609 | CB | ALA | H | 78 | 46.023 | −47.558 | 9.152 | 1.00 | 18.36 | C |
| ATOM | 610 | C | ALA | H | 78 | 43.741 | −48.548 | 9.397 | 1.00 | 19.01 | C |
| ATOM | 611 | O | ALA | H | 78 | 42.787 | −47.950 | 8.873 | 1.00 | 18.63 | O |
| ATOM | 612 | N | TYR | H | 79 | 43.657 | −49.151 | 10.585 | 1.00 | 18.65 | N |
| ATOM | 613 | CA | TYR | H | 79 | 42.388 | −49.218 | 11.317 | 1.00 | 18.45 | C |
| ATOM | 614 | CB | TYR | H | 79 | 41.796 | −50.631 | 11.282 | 1.00 | 18.63 | C |
| ATOM | 615 | CG | TYR | H | 79 | 41.457 | −51.168 | 9.913 | 1.00 | 19.32 | C |
| ATOM | 616 | CD1 | TYR | H | 79 | 40.226 | −50.876 | 9.319 | 1.00 | 20.11 | C |
| ATOM | 617 | CE1 | TYR | H | 79 | 39.897 | −51.372 | 8.067 | 1.00 | 21.27 | C |
| ATOM | 618 | CZ | TYR | H | 79 | 40.791 | −52.189 | 7.396 | 1.00 | 22.63 | C |
| ATOM | 619 | OH | TYR | H | 79 | 40.445 | −52.681 | 6.159 | 1.00 | 23.30 | O |
| ATOM | 620 | CE2 | TYR | H | 79 | 42.028 | −52.507 | 7.967 | 1.00 | 21.80 | C |
| ATOM | 621 | CD2 | TYR | H | 79 | 42.348 | −51.991 | 9.226 | 1.00 | 19.84 | C |
| ATOM | 622 | C | TYR | H | 79 | 42.589 | −48.844 | 12.764 | 1.00 | 18.47 | C |
| ATOM | 623 | O | TYR | H | 79 | 43.639 | −49.140 | 13.349 | 1.00 | 18.15 | O |
| ATOM | 624 | N | LEU | H | 80 | 41.577 | −48.195 | 13.335 | 1.00 | 18.15 | N |
| ATOM | 625 | CA | LEU | H | 80 | 41.486 | −48.006 | 14.784 | 1.00 | 18.02 | C |
| ATOM | 626 | CB | LEU | H | 80 | 41.451 | −46.507 | 15.129 | 1.00 | 17.88 | C |
| ATOM | 627 | CG | LEU | H | 80 | 41.168 | −46.058 | 16.574 | 1.00 | 18.00 | C |
| ATOM | 628 | CD1 | LEU | H | 80 | 42.263 | −46.526 | 17.539 | 1.00 | 17.13 | C |
| ATOM | 629 | CD2 | LEU | H | 80 | 41.000 | −44.532 | 16.673 | 1.00 | 16.69 | C |
| ATOM | 630 | C | LEU | H | 80 | 40.226 | −48.722 | 15.314 | 1.00 | 18.47 | C |
| ATOM | 631 | O | LEU | H | 80 | 39.104 | −48.448 | 14.864 | 1.00 | 17.93 | O |
| ATOM | 632 | N | GLN | H | 81 | 40.417 | −49.644 | 16.247 | 1.00 | 18.65 | N |
| ATOM | 633 | CA | GLN | H | 81 | 39.310 | −50.245 | 16.969 | 1.00 | 19.97 | C |
| ATOM | 634 | CB | GLN | H | 81 | 39.476 | −51.757 | 17.121 | 1.00 | 20.04 | C |
| ATOM | 635 | CG | GLN | H | 81 | 39.110 | −52.562 | 15.910 | 1.00 | 22.66 | C |
| ATOM | 636 | CD | GLN | H | 81 | 39.687 | −53.973 | 15.961 | 1.00 | 25.81 | C |
| ATOM | 637 | OE1 | GLN | H | 81 | 38.943 | −54.945 | 16.048 | 1.00 | 26.76 | O |
| ATOM | 638 | NE2 | GLN | H | 81 | 41.020 | −54.085 | 15.914 | 1.00 | 25.96 | N |
| ATOM | 639 | C | GLN | H | 81 | 39.277 | −49.630 | 18.341 | 1.00 | 20.31 | C |
| ATOM | 640 | O | GLN | H | 81 | 40.323 | −49.378 | 18.939 | 1.00 | 20.72 | O |
| ATOM | 641 | N | TRP | H | 82 | 38.073 | −49.393 | 18.839 | 1.00 | 20.84 | N |
| ATOM | 642 | CA | TRP | H | 82 | 37.886 | −48.832 | 20.170 | 1.00 | 21.33 | C |
| ATOM | 643 | CB | TRP | H | 82 | 37.535 | −47.345 | 20.083 | 1.00 | 21.45 | C |
| ATOM | 644 | CG | TRP | H | 82 | 37.857 | −46.560 | 21.325 | 1.00 | 21.63 | C |
| ATOM | 645 | CD1 | TRP | H | 82 | 37.183 | −46.588 | 22.514 | 1.00 | 24.39 | C |
| ATOM | 646 | NE1 | TRP | H | 82 | 37.770 | −45.732 | 23.421 | 1.00 | 24.78 | N |
| ATOM | 647 | CE2 | TRP | H | 82 | 38.837 | −45.120 | 22.816 | 1.00 | 24.78 | C |
| ATOM | 648 | CD2 | TRP | H | 82 | 38.927 | −45.626 | 21.494 | 1.00 | 23.08 | C |
| ATOM | 649 | CE3 | TRP | H | 82 | 39.950 | −45.157 | 20.655 | 1.00 | 22.50 | C |
| ATOM | 650 | CZ3 | TRP | H | 82 | 40.846 | −44.226 | 21.156 | 1.00 | 21.71 | C |
| ATOM | 651 | CH2 | TRP | H | 82 | 40.732 | −43.738 | 22.475 | 1.00 | 23.80 | C |
| ATOM | 652 | CZ2 | TRP | H | 82 | 39.742 | −44.177 | 23.324 | 1.00 | 24.12 | C |
| ATOM | 653 | C | TRP | H | 82 | 36.755 | −49.620 | 20.822 | 1.00 | 21.58 | C |
| ATOM | 654 | O | TRP | H | 82 | 35.635 | −49.628 | 20.314 | 1.00 | 21.76 | O |
| ATOM | 655 | N | SER | H | 82A | 37.054 | −50.290 | 21.930 | 1.00 | 21.46 | N |
| ATOM | 656 | CA | SER | H | 82A | 36.057 | −51.081 | 22.647 | 1.00 | 21.41 | C |
| ATOM | 657 | CB | SER | H | 82A | 36.657 | −52.419 | 23.080 | 1.00 | 21.55 | C |
| ATOM | 658 | OG | SER | H | 82A | 37.766 | −52.203 | 23.938 | 1.00 | 22.39 | O |
| ATOM | 659 | C | SER | H | 82A | 35.520 | −50.331 | 23.871 | 1.00 | 21.48 | C |
| ATOM | 660 | O | SER | H | 82A | 36.135 | −49.360 | 24.338 | 1.00 | 21.46 | O |
| ATOM | 661 | N | SER | H | 82B | 34.379 | −50.792 | 24.386 | 1.00 | 20.88 | N |
| ATOM | 662 | CA | SER | H | 82B | 33.736 | −50.179 | 25.551 | 1.00 | 21.28 | C |
| ATOM | 663 | CB | SER | H | 82B | 34.494 | −50.520 | 26.837 | 1.00 | 21.35 | C |
| ATOM | 664 | OG | SER | H | 82B | 34.503 | −51.910 | 27.063 | 1.00 | 21.93 | O |
| ATOM | 665 | C | SER | H | 82B | 33.635 | −48.668 | 25.428 | 1.00 | 21.07 | C |
| ATOM | 666 | O | SER | H | 82B | 34.155 | −47.940 | 26.278 | 1.00 | 21.21 | O |
| ATOM | 667 | N | LEU | H | 82C | 32.973 | −48.197 | 24.375 | 1.00 | 20.44 | N |
| ATOM | 668 | CA | LEU | H | 82C | 32.882 | −46.767 | 24.120 | 1.00 | 20.46 | C |
| ATOM | 669 | CB | LEU | H | 82C | 32.199 | −46.494 | 22.786 | 1.00 | 20.38 | C |
| ATOM | 670 | CG | LEU | H | 82C | 33.040 | −46.761 | 21.535 | 1.00 | 21.03 | C |
| ATOM | 671 | CD1 | LEU | H | 82C | 32.108 | −46.904 | 20.341 | 1.00 | 20.49 | C |
| ATOM | 672 | CD2 | LEU | H | 82C | 34.063 | −45.661 | 21.295 | 1.00 | 19.38 | C |
| ATOM | 673 | C | LEU | H | 82C | 32.112 | −46.074 | 25.222 | 1.00 | 20.50 | C |
| ATOM | 674 | O | LEU | H | 82C | 31.140 | −46.617 | 25.742 | 1.00 | 19.62 | O |
| ATOM | 675 | N | LYS | H | 83 | 32.569 | −44.879 | 25.568 | 1.00 | 20.33 | N |
| ATOM | 676 | CA | LYS | H | 83 | 31.896 | −44.040 | 26.542 | 1.00 | 20.73 | C |
| ATOM | 677 | CB | LYS | H | 83 | 32.840 | −43.733 | 27.697 | 1.00 | 21.32 | C |
| ATOM | 678 | CG | LYS | H | 83 | 33.170 | −44.930 | 28.581 | 1.00 | 23.87 | C |
| ATOM | 679 | CD | LYS | H | 83 | 34.566 | −44.823 | 29.176 | 1.00 | 26.98 | C |
| ATOM | 680 | CE | LYS | H | 83 | 34.765 | −43.534 | 29.972 | 1.00 | 29.30 | C |
| ATOM | 681 | NZ | LYS | H | 83 | 35.824 | −43.706 | 31.024 | 1.00 | 32.21 | N |
| ATOM | 682 | C | LYS | H | 83 | 31.457 | −42.742 | 25.877 | 1.00 | 20.38 | C |
| ATOM | 683 | O | LYS | H | 83 | 31.978 | −42.381 | 24.807 | 1.00 | 19.25 | O |
| ATOM | 684 | N | ALA | H | 84 | 30.506 | −42.041 | 26.503 | 1.00 | 20.39 | N |
| ATOM | 685 | CA | ALA | H | 84 | 30.048 | −40.742 | 25.991 | 1.00 | 20.74 | C |
| ATOM | 686 | CB | ALA | H | 84 | 28.958 | −40.140 | 26.890 | 1.00 | 21.05 | C |
| ATOM | 687 | C | ALA | H | 84 | 31.219 | −39.770 | 25.820 | 1.00 | 20.89 | C |
| ATOM | 688 | O | ALA | H | 84 | 31.264 | −39.017 | 24.859 | 1.00 | 21.06 | O |
| ATOM | 689 | N | SER | H | 85 | 32.179 | −39.831 | 26.739 | 1.00 | 21.81 | N |
| ATOM | 690 | CA | SER | H | 85 | 33.377 | −38.984 | 26.707 | 1.00 | 22.32 | C |
| ATOM | 691 | CB | SER | H | 85 | 34.080 | −38.980 | 28.084 | 1.00 | 23.15 | C |
| ATOM | 692 | OG | SER | H | 85 | 34.721 | −40.213 | 28.375 | 1.00 | 23.00 | O |
| ATOM | 693 | C | SER | H | 85 | 34.356 | −39.353 | 25.585 | 1.00 | 22.70 | C |
| ATOM | 694 | O | SER | H | 85 | 35.348 | −38.645 | 25.366 | 1.00 | 23.04 | O |
| ATOM | 695 | N | ASP | H | 86 | 34.075 | −40.447 | 24.867 | 1.00 | 22.02 | N |
| ATOM | 696 | CA | ASP | H | 86 | 34.836 | −40.789 | 23.660 | 1.00 | 21.77 | C |
| ATOM | 697 | CB | ASP | H | 86 | 34.904 | −42.309 | 23.460 | 1.00 | 22.12 | C |
| ATOM | 698 | CG | ASP | H | 86 | 35.632 | −43.022 | 24.609 | 1.00 | 23.31 | C |
| ATOM | 699 | OD1 | ASP | H | 86 | 35.153 | −44.096 | 25.043 | 1.00 | 23.70 | O |
| ATOM | 700 | OD2 | ASP | H | 86 | 36.688 | −42.512 | 25.072 | 1.00 | 23.15 | O |
| ATOM | 701 | C | ASP | H | 86 | 34.311 | −40.073 | 22.403 | 1.00 | 21.15 | C |
| ATOM | 702 | O | ASP | H | 86 | 34.879 | −40.218 | 21.312 | 1.00 | 20.96 | O |
| ATOM | 703 | N | THR | H | 87 | 33.255 | −39.278 | 22.567 | 1.00 | 20.62 | N |
| ATOM | 704 | CA | THR | H | 87 | 32.719 | −38.451 | 21.476 | 1.00 | 20.31 | C |
| ATOM | 705 | CB | THR | H | 87 | 31.431 | −37.708 | 21.923 | 1.00 | 20.54 | C |
| ATOM | 706 | OG1 | THR | H | 87 | 30.396 | −38.660 | 22.237 | 1.00 | 18.87 | O |
| ATOM | 707 | CG2 | THR | H | 87 | 30.948 | −36.718 | 20.838 | 1.00 | 20.82 | C |
| ATOM | 708 | C | THR | H | 87 | 33.787 | −37.449 | 21.025 | 1.00 | 20.94 | C |
| ATOM | 709 | O | THR | H | 87 | 34.277 | −36.646 | 21.836 | 1.00 | 20.70 | O |
| ATOM | 710 | N | ALA | H | 88 | 34.170 | −37.526 | 19.746 | 1.00 | 20.12 | N |
| ATOM | 711 | CA | ALA | H | 88 | 35.306 | −36.775 | 19.236 | 1.00 | 19.93 | C |
| ATOM | 712 | CB | ALA | H | 88 | 36.618 | −37.207 | 19.951 | 1.00 | 19.92 | C |
| ATOM | 713 | C | ALA | H | 88 | 35.469 | −36.926 | 17.729 | 1.00 | 19.54 | C |
| ATOM | 714 | O | ALA | H | 88 | 34.911 | −37.843 | 17.097 | 1.00 | 19.28 | O |
| ATOM | 715 | N | MET | H | 89 | 36.247 | −36.015 | 17.160 | 1.00 | 19.24 | N |
| ATOM | 716 | CA | MET | H | 89 | 36.753 | −36.191 | 15.817 | 1.00 | 19.19 | C |
| ATOM | 717 | CB | MET | H | 89 | 37.064 | −34.841 | 15.172 | 1.00 | 19.77 | C |
| ATOM | 718 | CG | MET | H | 89 | 37.440 | −34.944 | 13.689 | 1.00 | 20.43 | C |
| ATOM | 719 | SD | MET | H | 89 | 36.142 | −35.520 | 12.576 | 1.00 | 24.47 | S |
| ATOM | 720 | CE | MET | H | 89 | 35.132 | −34.031 | 12.430 | 1.00 | 26.69 | C |
| ATOM | 721 | C | MET | H | 89 | 38.031 | −37.022 | 15.905 | 1.00 | 18.78 | C |
| ATOM | 722 | O | MET | H | 89 | 38.963 | −36.639 | 16.590 | 1.00 | 18.57 | O |
| ATOM | 723 | N | TYR | H | 90 | 38.060 | −38.148 | 15.198 | 1.00 | 18.25 | N |
| ATOM | 724 | CA | TYR | H | 90 | 39.224 | −39.040 | 15.174 | 1.00 | 18.41 | C |
| ATOM | 725 | CB | TYR | H | 90 | 38.781 | −40.510 | 15.372 | 1.00 | 17.63 | C |
| ATOM | 726 | CG | TYR | H | 90 | 38.328 | −40.805 | 16.797 | 1.00 | 18.08 | C |
| ATOM | 727 | CD1 | TYR | H | 90 | 37.095 | −40.344 | 17.272 | 1.00 | 15.92 | C |
| ATOM | 728 | CE1 | TYR | H | 90 | 36.678 | −40.596 | 18.569 | 1.00 | 16.07 | C |
| ATOM | 729 | CZ | TYR | H | 90 | 37.499 | −41.313 | 19.414 | 1.00 | 15.22 | C |
| ATOM | 730 | OH | TYR | H | 90 | 37.099 | −41.556 | 20.713 | 1.00 | 17.89 | O |
| ATOM | 731 | CE2 | TYR | H | 90 | 38.728 | −41.790 | 18.966 | 1.00 | 16.70 | C |
| ATOM | 732 | CD2 | TYR | H | 90 | 39.133 | −41.545 | 17.670 | 1.00 | 14.55 | C |
| ATOM | 733 | C | TYR | H | 90 | 39.995 | −38.860 | 13.861 | 1.00 | 18.57 | C |
| ATOM | 734 | O | TYR | H | 90 | 39.465 | −39.120 | 12.789 | 1.00 | 20.09 | O |
| ATOM | 735 | N | PHE | H | 91 | 41.244 | −38.406 | 13.945 | 1.00 | 18.57 | N |
| ATOM | 736 | CA | PHE | H | 91 | 42.092 | −38.262 | 12.748 | 1.00 | 18.53 | C |
| ATOM | 737 | CB | PHE | H | 91 | 42.877 | −36.947 | 12.804 | 1.00 | 17.87 | C |
| ATOM | 738 | CG | PHE | H | 91 | 42.032 | −35.705 | 12.737 | 1.00 | 18.55 | C |
| ATOM | 739 | CD1 | PHE | H | 91 | 41.568 | −35.225 | 11.502 | 1.00 | 20.17 | C |
| ATOM | 740 | CE1 | PHE | H | 91 | 40.805 | −34.051 | 11.425 | 1.00 | 19.28 | C |
| ATOM | 741 | CZ | PHE | H | 91 | 40.528 | −33.325 | 12.582 | 1.00 | 19.87 | C |
| ATOM | 742 | CE2 | PHE | H | 91 | 41.004 | −33.785 | 13.831 | 1.00 | 20.43 | C |
| ATOM | 743 | CD2 | PHE | H | 91 | 41.763 | −34.971 | 13.890 | 1.00 | 19.47 | C |
| ATOM | 744 | C | PHE | H | 91 | 43.142 | −39.383 | 12.640 | 1.00 | 18.52 | C |
| ATOM | 745 | O | PHE | H | 91 | 43.677 | −39.828 | 13.647 | 1.00 | 18.02 | O |
| ATOM | 746 | N | CYS | H | 92 | 43.456 | −39.790 | 11.413 | 1.00 | 18.30 | N |
| ATOM | 747 | CA | CYS | H | 92 | 44.711 | −40.487 | 11.153 | 1.00 | 19.10 | C |
| ATOM | 748 | CB | CYS | H | 92 | 44.536 | −41.744 | 10.282 | 1.00 | 19.32 | C |
| ATOM | 749 | SG | CYS | H | 92 | 43.884 | −41.452 | 8.643 | 1.00 | 23.42 | S |
| ATOM | 750 | C | CYS | H | 92 | 45.629 | −39.492 | 10.478 | 1.00 | 17.94 | C |
| ATOM | 751 | O | CYS | H | 92 | 45.166 | −38.565 | 9.814 | 1.00 | 19.10 | O |
| ATOM | 752 | N | ALA | H | 93 | 46.928 | −39.657 | 10.666 | 1.00 | 16.65 | N |
| ATOM | 753 | CA | ALA | H | 93 | 47.884 | −38.720 | 10.095 | 1.00 | 16.07 | C |
| ATOM | 754 | CB | ALA | H | 93 | 48.121 | −37.541 | 11.045 | 1.00 | 15.15 | C |
| ATOM | 755 | C | ALA | H | 93 | 49.205 | −39.399 | 9.775 | 1.00 | 16.46 | C |
| ATOM | 756 | O | ALA | H | 93 | 49.609 | −40.345 | 10.452 | 1.00 | 16.70 | O |
| ATOM | 757 | N | ARG | H | 94 | 49.874 | −38.919 | 8.739 | 1.00 | 16.60 | N |
| ATOM | 758 | CA | ARG | H | 94 | 51.236 | −39.356 | 8.484 | 1.00 | 16.76 | C |
| ATOM | 759 | CB | ARG | H | 94 | 51.363 | −39.989 | 7.097 | 1.00 | 17.06 | C |
| ATOM | 760 | CG | ARG | H | 94 | 52.782 | −40.479 | 6.759 | 1.00 | 19.19 | C |
| ATOM | 761 | CD | ARG | H | 94 | 52.842 | −41.050 | 5.360 | 1.00 | 21.88 | C |
| ATOM | 762 | NE | ARG | H | 94 | 54.214 | −41.371 | 4.969 | 1.00 | 22.65 | N |
| ATOM | 763 | CZ | ARG | H | 94 | 54.697 | −41.222 | 3.744 | 1.00 | 22.55 | C |
| ATOM | 764 | NH1 | ARG | H | 94 | 53.934 | −40.744 | 2.767 | 1.00 | 25.64 | N |
| ATOM | 765 | NH2 | ARG | H | 94 | 55.942 | −41.557 | 3.488 | 1.00 | 23.39 | N |
| ATOM | 766 | C | ARG | H | 94 | 52.087 | −38.115 | 8.628 | 1.00 | 16.67 | C |
| ATOM | 767 | O | ARG | H | 94 | 51.949 | −37.183 | 7.845 | 1.00 | 16.54 | O |
| ATOM | 768 | N | ARG | H | 95 | 52.924 | −38.073 | 9.668 | 1.00 | 15.78 | N |
| ATOM | 769 | CA | ARG | H | 95 | 53.746 | −36.878 | 9.931 | 1.00 | 16.23 | C |
| ATOM | 770 | CB | ARG | H | 95 | 53.467 | −36.336 | 11.344 | 1.00 | 15.92 | C |
| ATOM | 771 | CG | ARG | H | 95 | 51.984 | −36.061 | 11.634 | 1.00 | 15.80 | C |
| ATOM | 772 | CD | ARG | H | 95 | 51.777 | −35.286 | 12.904 | 1.00 | 15.58 | C |
| ATOM | 773 | NE | ARG | H | 95 | 52.116 | −36.013 | 14.144 | 1.00 | 15.22 | N |
| ATOM | 774 | CZ | ARG | H | 95 | 52.437 | −35.403 | 15.295 | 1.00 | 17.66 | C |
| ATOM | 775 | NH1 | ARG | H | 95 | 52.503 | −34.078 | 15.350 | 1.00 | 17.27 | N |
| ATOM | 776 | NH2 | ARG | H | 95 | 52.691 | −36.110 | 16.399 | 1.00 | 18.10 | N |
| ATOM | 777 | C | ARG | H | 95 | 55.245 | −37.118 | 9.791 | 1.00 | 16.90 | C |
| ATOM | 778 | O | ARG | H | 95 | 55.998 | −36.241 | 9.345 | 1.00 | 16.20 | O |
| ATOM | 779 | N | PHE | H | 96 | 55.677 | −38.305 | 10.200 | 1.00 | 16.39 | N |
| ATOM | 780 | CA | PHE | H | 96 | 57.097 | −38.597 | 10.291 | 1.00 | 16.86 | C |
| ATOM | 781 | CB | PHE | H | 96 | 57.468 | −38.810 | 11.772 | 1.00 | 16.65 | C |
| ATOM | 782 | CG | PHE | H | 96 | 56.955 | −37.714 | 12.680 | 1.00 | 15.17 | C |
| ATOM | 783 | CD1 | PHE | H | 96 | 57.422 | −36.411 | 12.546 | 1.00 | 15.88 | C |
| ATOM | 784 | CE1 | PHE | H | 96 | 56.957 | −35.367 | 13.379 | 1.00 | 16.95 | C |
| ATOM | 785 | CZ | PHE | H | 96 | 56.020 | −35.655 | 14.371 | 1.00 | 15.23 | C |
| ATOM | 786 | CE2 | PHE | H | 96 | 55.549 | −36.961 | 14.522 | 1.00 | 14.65 | C |
| ATOM | 787 | CD2 | PHE | H | 96 | 56.017 | −37.992 | 13.661 | 1.00 | 16.34 | C |
| ATOM | 788 | C | PHE | H | 96 | 57.492 | −39.822 | 9.498 | 1.00 | 16.90 | C |
| ATOM | 789 | O | PHE | H | 96 | 56.638 | −40.643 | 9.124 | 1.00 | 17.02 | O |
| ATOM | 790 | N | GLY | H | 97 | 58.794 | −39.922 | 9.235 | 1.00 | 16.91 | N |
| ATOM | 791 | CA | GLY | H | 97 | 59.408 | −41.165 | 8.784 | 1.00 | 16.88 | C |
| ATOM | 792 | C | GLY | H | 97 | 60.253 | −41.630 | 9.956 | 1.00 | 16.96 | C |
| ATOM | 793 | O | GLY | H | 97 | 60.107 | −41.120 | 11.061 | 1.00 | 15.67 | O |
| ATOM | 794 | N | TYR | H | 98 | 61.136 | −42.591 | 9.717 | 1.00 | 16.57 | N |
| ATOM | 795 | CA | TYR | H | 98 | 62.086 | −43.045 | 10.735 | 1.00 | 16.98 | C |
| ATOM | 796 | CB | TYR | H | 98 | 63.006 | −44.111 | 10.135 | 1.00 | 18.09 | C |
| ATOM | 797 | CG | TYR | H | 98 | 62.462 | −45.511 | 10.070 | 1.00 | 21.07 | C |
| ATOM | 798 | CD1 | TYR | H | 98 | 61.140 | −45.770 | 9.744 | 1.00 | 23.51 | C |
| ATOM | 799 | CE1 | TYR | H | 98 | 60.655 | −47.096 | 9.670 | 1.00 | 26.62 | C |
| ATOM | 800 | CZ | TYR | H | 98 | 61.519 | −48.152 | 9.925 | 1.00 | 28.45 | C |
| ATOM | 801 | OH | TYR | H | 98 | 61.087 | −49.466 | 9.869 | 1.00 | 30.65 | O |
| ATOM | 802 | CE2 | TYR | H | 98 | 62.853 | −47.902 | 10.251 | 1.00 | 27.73 | C |
| ATOM | 803 | CD2 | TYR | H | 98 | 63.308 | −46.597 | 10.321 | 1.00 | 26.19 | C |
| ATOM | 804 | C | TYR | H | 98 | 62.966 | −41.897 | 11.213 | 1.00 | 15.99 | C |
| ATOM | 805 | O | TYR | H | 98 | 63.319 | −41.026 | 10.435 | 1.00 | 15.71 | O |
| ATOM | 806 | N | TYR | H | 99 | 63.360 | −41.910 | 12.486 | 1.00 | 15.73 | N |
| ATOM | 807 | CA | TYR | H | 99 | 64.309 | −40.928 | 12.975 | 1.00 | 15.07 | C |
| ATOM | 808 | CB | TYR | H | 99 | 64.742 | −41.260 | 14.390 | 1.00 | 15.39 | C |
| ATOM | 809 | CG | TYR | H | 99 | 63.712 | −41.017 | 15.455 | 1.00 | 14.90 | C |
| ATOM | 810 | CD1 | TYR | H | 99 | 63.288 | −39.722 | 15.747 | 1.00 | 16.50 | C |
| ATOM | 811 | CE1 | TYR | H | 99 | 62.358 | −39.478 | 16.750 | 1.00 | 13.13 | C |
| ATOM | 812 | CZ | TYR | H | 99 | 61.832 | −40.538 | 17.465 | 1.00 | 14.91 | C |
| ATOM | 813 | OH | TYR | H | 99 | 60.916 | −40.259 | 18.457 | 1.00 | 14.83 | O |
| ATOM | 814 | CE2 | TYR | H | 99 | 62.238 | −41.837 | 17.224 | 1.00 | 14.08 | C |
| ATOM | 815 | CD2 | TYR | H | 99 | 63.190 | −42.075 | 16.203 | 1.00 | 16.25 | C |
| ATOM | 816 | C | TYR | H | 99 | 65.543 | −40.986 | 12.102 | 1.00 | 15.51 | C |
| ATOM | 817 | O | TYR | H | 99 | 66.004 | −42.081 | 11.778 | 1.00 | 15.21 | O |
| ATOM | 818 | N | GLY | H | 100 | 66.084 | −39.827 | 11.741 | 1.00 | 14.57 | N |
| ATOM | 819 | CA | GLY | H | 100 | 67.255 | −39.785 | 10.873 | 1.00 | 15.06 | C |
| ATOM | 820 | C | GLY | H | 100 | 66.930 | −39.683 | 9.384 | 1.00 | 15.49 | C |
| ATOM | 821 | O | GLY | H | 100 | 67.824 | −39.426 | 8.577 | 1.00 | 15.93 | O |
| ATOM | 822 | N | SER | H | 100A | 65.659 | −39.856 | 9.026 | 1.00 | 15.94 | N |
| ATOM | 823 | CA | SER | H | 100A | 65.237 | −39.866 | 7.621 | 1.00 | 16.92 | C |
| ATOM | 824 | CB | SER | H | 100A | 63.876 | −40.570 | 7.462 | 1.00 | 17.19 | C |
| ATOM | 825 | OG | SER | H | 100A | 62.819 | −39.864 | 8.130 | 1.00 | 18.18 | O |
| ATOM | 826 | C | SER | H | 100A | 65.161 | −38.467 | 7.000 | 1.00 | 17.59 | C |
| ATOM | 827 | O | SER | H | 100A | 65.145 | −38.338 | 5.775 | 1.00 | 17.56 | O |
| ATOM | 828 | N | GLY | H | 100B | 65.126 | −37.429 | 7.839 | 1.00 | 16.78 | N |
| ATOM | 829 | CA | GLY | H | 100B | 64.909 | −36.069 | 7.361 | 1.00 | 18.16 | C |
| ATOM | 830 | C | GLY | H | 100B | 63.465 | −35.811 | 6.931 | 1.00 | 18.37 | C |
| ATOM | 831 | O | GLY | H | 100B | 63.147 | −34.718 | 6.495 | 1.00 | 18.56 | O |
| ATOM | 832 | N | ASN | H | 100C | 62.594 | −36.818 | 7.048 | 1.00 | 18.14 | N |
| ATOM | 833 | CA | ASN | H | 100C | 61.210 | −36.732 | 6.573 | 1.00 | 18.79 | C |
| ATOM | 834 | CB | ASN | H | 100C | 60.664 | −38.124 | 6.229 | 1.00 | 19.65 | C |
| ATOM | 835 | CG | ASN | H | 100C | 61.338 | −38.732 | 5.002 | 1.00 | 22.25 | C |
| ATOM | 836 | OD1 | ASN | H | 100C | 61.756 | −38.021 | 4.077 | 1.00 | 24.92 | O |
| ATOM | 837 | ND2 | ASN | H | 100C | 61.425 | −40.050 | 4.984 | 1.00 | 24.80 | N |
| ATOM | 838 | C | ASN | H | 100C | 60.210 | −36.053 | 7.504 | 1.00 | 18.31 | C |
| ATOM | 839 | O | ASN | H | 100C | 59.971 | −36.515 | 8.642 | 1.00 | 18.17 | O |
| ATOM | 840 | N | TYR | H | 100D | 59.605 | −34.984 | 6.987 | 1.00 | 17.34 | N |
| ATOM | 841 | CA | TYR | H | 100D | 58.483 | −34.320 | 7.630 | 1.00 | 17.68 | C |
| ATOM | 842 | CB | TYR | H | 100D | 58.912 | −32.972 | 8.252 | 1.00 | 17.33 | C |
| ATOM | 843 | CG | TYR | H | 100D | 59.881 | −33.254 | 9.373 | 1.00 | 17.03 | C |
| ATOM | 844 | CD1 | TYR | H | 100D | 59.417 | −33.523 | 10.667 | 1.00 | 17.50 | C |
| ATOM | 845 | CE1 | TYR | H | 100D | 60.307 | −33.863 | 11.686 | 1.00 | 16.12 | C |
| ATOM | 846 | CZ | TYR | H | 100D | 61.660 | −33.950 | 11.402 | 1.00 | 17.21 | C |
| ATOM | 847 | OH | TYR | H | 100D | 62.532 | −34.273 | 12.395 | 1.00 | 18.26 | O |
| ATOM | 848 | CE2 | TYR | H | 100D | 62.142 | −33.708 | 10.127 | 1.00 | 16.21 | C |
| ATOM | 849 | CD2 | TYR | H | 100D | 61.249 | −33.374 | 9.120 | 1.00 | 16.06 | C |
| ATOM | 850 | C | TYR | H | 100D | 57.347 | −34.209 | 6.631 | 1.00 | 17.74 | C |
| ATOM | 851 | O | TYR | H | 100D | 57.488 | −33.611 | 5.577 | 1.00 | 16.67 | O |
| ATOM | 852 | N | PHE | H | 100E | 56.242 | −34.861 | 6.975 | 1.00 | 18.90 | N |
| ATOM | 853 | CA | PHE | H | 100E | 55.057 | −34.907 | 6.147 | 1.00 | 20.12 | C |
| ATOM | 854 | CB | PHE | H | 100E | 54.613 | −36.368 | 5.914 | 1.00 | 20.16 | C |
| ATOM | 855 | CG | PHE | H | 100E | 55.686 | −37.247 | 5.376 | 1.00 | 20.37 | C |
| ATOM | 856 | CD1 | PHE | H | 100E | 56.198 | −37.040 | 4.086 | 1.00 | 22.32 | C |
| ATOM | 857 | CE1 | PHE | H | 100E | 57.216 | −37.861 | 3.573 | 1.00 | 21.76 | C |
| ATOM | 858 | CZ | PHE | H | 100E | 57.725 | −38.902 | 4.354 | 1.00 | 20.48 | C |
| ATOM | 859 | CE2 | PHE | H | 100E | 57.221 | −39.117 | 5.641 | 1.00 | 21.06 | C |
| ATOM | 860 | CD2 | PHE | H | 100E | 56.195 | −38.288 | 6.144 | 1.00 | 20.62 | C |
| ATOM | 861 | C | PHE | H | 100E | 53.997 | −34.186 | 6.922 | 1.00 | 20.50 | C |
| ATOM | 862 | O | PHE | H | 100E | 54.125 | −33.976 | 8.130 | 1.00 | 21.40 | O |
| ATOM | 863 | N | ASP | H | 101 | 52.947 | −33.772 | 6.244 | 1.00 | 21.06 | N |
| ATOM | 864 | CA | ASP | H | 101 | 51.818 | −33.231 | 6.975 | 1.00 | 20.66 | C |
| ATOM | 865 | CB | ASP | H | 101 | 51.863 | −31.688 | 6.982 | 1.00 | 20.50 | C |
| ATOM | 866 | CG | ASP | H | 101 | 50.914 | −31.069 | 8.011 | 1.00 | 19.88 | C |
| ATOM | 867 | OD1 | ASP | H | 101 | 50.767 | −31.603 | 9.142 | 1.00 | 20.29 | O |
| ATOM | 868 | OD2 | ASP | H | 101 | 50.313 | −30.032 | 7.684 | 1.00 | 18.63 | O |
| ATOM | 869 | C | ASP | H | 101 | 50.558 | −33.769 | 6.306 | 1.00 | 21.09 | C |
| ATOM | 870 | O | ASP | H | 101 | 49.723 | −33.001 | 5.845 | 1.00 | 21.28 | O |
| ATOM | 871 | N | TYR | H | 102 | 50.440 | −35.098 | 6.243 | 1.00 | 20.84 | N |
| ATOM | 872 | CA | TYR | H | 102 | 49.237 | −35.722 | 5.699 | 1.00 | 21.07 | C |
| ATOM | 873 | CB | TYR | H | 102 | 49.585 | −36.984 | 4.903 | 1.00 | 21.64 | C |
| ATOM | 874 | CG | TYR | H | 102 | 50.591 | −36.738 | 3.799 | 1.00 | 24.17 | C |
| ATOM | 875 | CD1 | TYR | H | 102 | 50.354 | −35.783 | 2.820 | 1.00 | 27.46 | C |
| ATOM | 876 | CE1 | TYR | H | 102 | 51.272 | −35.558 | 1.791 | 1.00 | 30.22 | C |
| ATOM | 877 | CZ | TYR | H | 102 | 52.443 | −36.298 | 1.743 | 1.00 | 28.79 | C |
| ATOM | 878 | OH | TYR | H | 102 | 53.338 | −36.065 | 0.724 | 1.00 | 30.74 | O |
| ATOM | 879 | CE2 | TYR | H | 102 | 52.701 | −37.256 | 2.702 | 1.00 | 27.24 | C |
| ATOM | 880 | CD2 | TYR | H | 102 | 51.774 | −37.476 | 3.721 | 1.00 | 26.02 | C |
| ATOM | 881 | C | TYR | H | 102 | 48.236 | −36.018 | 6.805 | 1.00 | 20.29 | C |
| ATOM | 882 | O | TYR | H | 102 | 48.549 | −36.711 | 7.759 | 1.00 | 19.79 | O |
| ATOM | 883 | N | TRP | H | 103 | 47.037 | −35.455 | 6.684 | 1.00 | 19.71 | N |
| ATOM | 884 | CA | TRP | H | 103 | 45.956 | −35.732 | 7.628 | 1.00 | 19.91 | C |
| ATOM | 885 | CB | TRP | H | 103 | 45.527 | −34.441 | 8.343 | 1.00 | 20.24 | C |
| ATOM | 886 | CG | TRP | H | 103 | 46.563 | −33.899 | 9.260 | 1.00 | 19.46 | C |
| ATOM | 887 | CD1 | TRP | H | 103 | 47.684 | −33.209 | 8.911 | 1.00 | 20.13 | C |
| ATOM | 888 | NE1 | TRP | H | 103 | 48.410 | −32.884 | 10.029 | 1.00 | 18.67 | N |
| ATOM | 889 | CE2 | TRP | H | 103 | 47.747 | −33.349 | 11.133 | 1.00 | 19.36 | C |
| ATOM | 890 | CD2 | TRP | H | 103 | 46.577 | −34.001 | 10.680 | 1.00 | 19.19 | C |
| ATOM | 891 | CE3 | TRP | H | 103 | 45.717 | −34.586 | 11.621 | 1.00 | 18.29 | C |
| ATOM | 892 | CZ3 | TRP | H | 103 | 46.037 | −34.476 | 12.965 | 1.00 | 19.04 | C |
| ATOM | 893 | CH2 | TRP | H | 103 | 47.210 | −33.834 | 13.384 | 1.00 | 19.01 | C |
| ATOM | 894 | CZ2 | TRP | H | 103 | 48.077 | −33.261 | 12.486 | 1.00 | 18.62 | C |
| ATOM | 895 | C | TRP | H | 103 | 44.755 | −36.293 | 6.866 | 1.00 | 20.16 | C |
| ATOM | 896 | O | TRP | H | 103 | 44.430 | −35.806 | 5.778 | 1.00 | 19.63 | O |
| ATOM | 897 | N | GLY | H | 104 | 44.110 | −37.314 | 7.423 | 1.00 | 20.33 | N |
| ATOM | 898 | CA | GLY | H | 104 | 42.788 | −37.715 | 6.928 | 1.00 | 20.25 | C |
| ATOM | 899 | C | GLY | H | 104 | 41.801 | −36.581 | 7.166 | 1.00 | 20.57 | C |
| ATOM | 900 | O | GLY | H | 104 | 42.098 | −35.646 | 7.900 | 1.00 | 19.42 | O |
| ATOM | 901 | N | GLN | H | 105 | 40.613 | −36.674 | 6.566 | 1.00 | 20.90 | N |
| ATOM | 902 | CA | GLN | H | 105 | 39.574 | −35.666 | 6.784 | 1.00 | 20.96 | C |
| ATOM | 903 | CB | GLN | H | 105 | 38.555 | −35.697 | 5.634 | 1.00 | 21.35 | C |
| ATOM | 904 | CG | GLN | H | 105 | 37.427 | −36.730 | 5.784 | 1.00 | 21.88 | C |
| ATOM | 905 | CD | GLN | H | 105 | 37.796 | −38.133 | 5.312 | 1.00 | 21.55 | C |
| ATOM | 906 | OE1 | GLN | H | 105 | 38.983 | −38.506 | 5.229 | 1.00 | 20.61 | O |
| ATOM | 907 | NE2 | GLN | H | 105 | 36.766 | −38.934 | 5.009 | 1.00 | 19.23 | N |
| ATOM | 908 | C | GLN | H | 105 | 38.900 | −35.803 | 8.161 | 1.00 | 21.36 | C |
| ATOM | 909 | O | GLN | H | 105 | 38.093 | −34.960 | 8.560 | 1.00 | 20.36 | O |
| ATOM | 910 | N | GLY | H | 106 | 39.234 | −36.868 | 8.885 | 1.00 | 21.08 | N |
| ATOM | 911 | CA | GLY | H | 106 | 38.683 | −37.086 | 10.210 | 1.00 | 21.19 | C |
| ATOM | 912 | C | GLY | H | 106 | 37.401 | −37.905 | 10.192 | 1.00 | 21.62 | C |
| ATOM | 913 | O | GLY | H | 106 | 36.616 | −37.827 | 9.243 | 1.00 | 20.91 | O |
| ATOM | 914 | N | THR | H | 107 | 37.207 | −38.723 | 11.227 | 1.00 | 20.88 | N |
| ATOM | 915 | CA | THR | H | 107 | 35.954 | −39.470 | 11.380 | 1.00 | 20.37 | C |
| ATOM | 916 | CB | THR | H | 107 | 36.188 | −41.008 | 11.466 | 1.00 | 20.69 | C |
| ATOM | 917 | OG1 | THR | H | 107 | 36.745 | −41.485 | 10.228 | 1.00 | 20.24 | O |
| ATOM | 918 | CG2 | THR | H | 107 | 34.865 | −41.749 | 11.737 | 1.00 | 17.25 | C |
| ATOM | 919 | C | THR | H | 107 | 35.258 | −38.977 | 12.640 | 1.00 | 20.72 | C |
| ATOM | 920 | O | THR | H | 107 | 35.780 | −39.145 | 13.751 | 1.00 | 20.41 | O |
| ATOM | 921 | N | MET | H | 108 | 34.097 | −38.353 | 12.471 | 1.00 | 20.46 | N |
| ATOM | 922 | CA | MET | H | 108 | 33.336 | −37.870 | 13.628 | 1.00 | 21.41 | C |
| ATOM | 923 | CB | MET | H | 108 | 32.333 | −36.756 | 13.250 | 1.00 | 21.67 | C |
| ATOM | 924 | CG | MET | H | 108 | 31.789 | −35.999 | 14.482 | 1.00 | 25.21 | C |
| ATOM | 925 | SD | MET | H | 108 | 33.123 | −35.215 | 15.460 | 1.00 | 31.86 | S |
| ATOM | 926 | CE | MET | H | 108 | 32.431 | −35.358 | 17.121 | 1.00 | 29.72 | C |
| ATOM | 927 | C | MET | H | 108 | 32.647 | −39.034 | 14.329 | 1.00 | 20.57 | C |
| ATOM | 928 | O | MET | H | 108 | 31.928 | −39.805 | 13.713 | 1.00 | 21.11 | O |
| ATOM | 929 | N | VAL | H | 109 | 32.892 | −39.175 | 15.625 | 1.00 | 20.69 | N |
| ATOM | 930 | CA | VAL | H | 109 | 32.332 | −40.290 | 16.372 | 1.00 | 20.22 | C |
| ATOM | 931 | CB | VAL | H | 109 | 33.437 | −41.209 | 16.952 | 1.00 | 20.27 | C |
| ATOM | 932 | CG1 | VAL | H | 109 | 32.831 | −42.208 | 17.916 | 1.00 | 19.83 | C |
| ATOM | 933 | CG2 | VAL | H | 109 | 34.195 | −41.930 | 15.824 | 1.00 | 20.34 | C |
| ATOM | 934 | C | VAL | H | 109 | 31.491 | −39.706 | 17.501 | 1.00 | 20.33 | C |
| ATOM | 935 | O | VAL | H | 109 | 31.993 | −38.903 | 18.285 | 1.00 | 19.45 | O |
| ATOM | 936 | N | THR | H | 110 | 30.209 | −40.091 | 17.540 | 1.00 | 19.99 | N |
| ATOM | 937 | CA | THR | H | 110 | 29.289 | −39.674 | 18.586 | 1.00 | 19.83 | C |
| ATOM | 938 | CB | THR | H | 110 | 28.015 | −38.974 | 18.009 | 1.00 | 19.95 | C |
| ATOM | 939 | OG1 | THR | H | 110 | 28.398 | −37.841 | 17.220 | 1.00 | 20.76 | O |
| ATOM | 940 | CG2 | THR | H | 110 | 27.089 | −38.510 | 19.136 | 1.00 | 19.76 | C |
| ATOM | 941 | C | THR | H | 110 | 28.887 | −40.900 | 19.381 | 1.00 | 19.80 | C |
| ATOM | 942 | O | THR | H | 110 | 28.398 | −41.888 | 18.816 | 1.00 | 20.60 | O |
| ATOM | 943 | N | VAL | H | 111 | 29.096 | −40.845 | 20.693 | 1.00 | 19.20 | N |
| ATOM | 944 | CA | VAL | H | 111 | 28.672 | −41.928 | 21.561 | 1.00 | 19.68 | C |
| ATOM | 945 | CB | VAL | H | 111 | 29.834 | −42.518 | 22.399 | 1.00 | 19.41 | C |
| ATOM | 946 | CG1 | VAL | H | 111 | 29.373 | −43.778 | 23.126 | 1.00 | 20.09 | C |
| ATOM | 947 | CG2 | VAL | H | 111 | 31.038 | −42.835 | 21.510 | 1.00 | 19.16 | C |
| ATOM | 948 | C | VAL | H | 111 | 27.553 | −41.428 | 22.473 | 1.00 | 20.31 | C |
| ATOM | 949 | O | VAL | H | 111 | 27.760 | −40.514 | 23.274 | 1.00 | 20.19 | O |
| ATOM | 950 | N | SER | H | 112 | 26.373 | −42.036 | 22.343 | 1.00 | 20.88 | N |
| ATOM | 951 | CA | SER | H | 112 | 25.182 | −41.608 | 23.085 | 1.00 | 21.37 | C |
| ATOM | 952 | CB | SER | H | 112 | 24.589 | −40.332 | 22.473 | 1.00 | 21.58 | C |
| ATOM | 953 | OG | SER | H | 112 | 23.365 | −39.985 | 23.112 | 1.00 | 22.58 | O |
| ATOM | 954 | C | SER | H | 112 | 24.116 | −42.689 | 23.095 | 1.00 | 21.98 | C |
| ATOM | 955 | O | SER | H | 112 | 24.000 | −43.471 | 22.147 | 1.00 | 22.36 | O |
| ATOM | 956 | N | SER | H | 113 | 23.330 | −42.720 | 24.167 | 1.00 | 22.25 | N |
| ATOM | 957 | CA | SER | H | 113 | 22.162 | −43.604 | 24.237 | 1.00 | 22.78 | C |
| ATOM | 958 | CB | SER | H | 113 | 21.792 | −43.895 | 25.692 | 1.00 | 22.77 | C |
| ATOM | 959 | OG | SER | H | 113 | 21.637 | −42.691 | 26.419 | 1.00 | 23.25 | O |
| ATOM | 960 | C | SER | H | 113 | 20.953 | −43.055 | 23.469 | 1.00 | 22.83 | C |
| ATOM | 961 | O | SER | H | 113 | 19.928 | −43.716 | 23.381 | 1.00 | 23.14 | O |
| ATOM | 962 | N | ALA | H | 114 | 21.081 | −41.864 | 22.885 | 1.00 | 23.14 | N |
| ATOM | 963 | CA | ALA | H | 114 | 19.961 | −41.245 | 22.164 | 1.00 | 23.11 | C |
| ATOM | 964 | CB | ALA | H | 114 | 20.198 | −39.755 | 21.986 | 1.00 | 22.95 | C |
| ATOM | 965 | C | ALA | H | 114 | 19.639 | −41.904 | 20.814 | 1.00 | 23.25 | C |
| ATOM | 966 | O | ALA | H | 114 | 20.496 | −42.540 | 20.192 | 1.00 | 23.03 | O |
| ATOM | 967 | N | SER | H | 115 | 18.394 | −41.739 | 20.375 | 1.00 | 23.48 | N |
| ATOM | 968 | CA | SER | H | 115 | 17.940 | −42.248 | 19.082 | 1.00 | 23.82 | C |
| ATOM | 969 | CB | SER | H | 115 | 16.614 | −42.993 | 19.243 | 1.00 | 24.00 | C |
| ATOM | 970 | OG | SER | H | 115 | 16.745 | −44.052 | 20.171 | 1.00 | 25.39 | O |
| ATOM | 971 | C | SER | H | 115 | 17.764 | −41.103 | 18.094 | 1.00 | 23.49 | C |
| ATOM | 972 | O | SER | H | 115 | 17.433 | −39.981 | 18.488 | 1.00 | 23.29 | O |
| ATOM | 973 | N | THR | H | 116 | 17.990 | −41.393 | 16.815 | 1.00 | 23.34 | N |
| ATOM | 974 | CA | THR | H | 116 | 17.740 | −40.439 | 15.743 | 1.00 | 22.95 | C |
| ATOM | 975 | CB | THR | H | 116 | 17.823 | −41.114 | 14.359 | 1.00 | 23.64 | C |
| ATOM | 976 | OG1 | THR | H | 116 | 19.061 | −41.829 | 14.253 | 1.00 | 23.30 | O |
| ATOM | 977 | CG2 | THR | H | 116 | 17.752 | −40.066 | 13.246 | 1.00 | 23.20 | C |
| ATOM | 978 | C | THR | H | 116 | 16.375 | −39.767 | 15.900 | 1.00 | 22.72 | C |
| ATOM | 979 | O | THR | H | 116 | 15.370 | −40.417 | 16.178 | 1.00 | 22.03 | O |
| ATOM | 980 | N | LYS | H | 117 | 16.368 | −38.449 | 15.739 | 1.00 | 22.54 | N |
| ATOM | 981 | CA | LYS | H | 117 | 15.167 | −37.641 | 15.828 | 1.00 | 22.43 | C |
| ATOM | 982 | CB | LYS | H | 117 | 14.838 | −37.300 | 17.288 | 1.00 | 22.35 | C |
| ATOM | 983 | CG | LYS | H | 117 | 13.590 | −36.424 | 17.427 | 1.00 | 23.34 | C |
| ATOM | 984 | CD | LYS | H | 117 | 13.335 | −35.988 | 18.862 | 1.00 | 25.67 | C |
| ATOM | 985 | CE | LYS | H | 117 | 12.148 | −35.024 | 18.944 | 1.00 | 27.44 | C |
| ATOM | 986 | NZ | LYS | H | 117 | 12.338 | −33.782 | 18.111 | 1.00 | 27.14 | N |
| ATOM | 987 | C | LYS | H | 117 | 15.385 | −36.357 | 15.042 | 1.00 | 22.24 | C |
| ATOM | 988 | O | LYS | H | 117 | 16.409 | −35.686 | 15.211 | 1.00 | 21.71 | O |
| ATOM | 989 | N | GLY | H | 118 | 14.413 | −36.016 | 14.198 | 1.00 | 21.87 | N |
| ATOM | 990 | CA | GLY | H | 118 | 14.426 | −34.743 | 13.480 | 1.00 | 21.71 | C |
| ATOM | 991 | C | GLY | H | 118 | 14.094 | −33.561 | 14.379 | 1.00 | 21.20 | C |
| ATOM | 992 | O | GLY | H | 118 | 13.404 | −33.723 | 15.375 | 1.00 | 20.52 | O |
| ATOM | 993 | N | PRO | H | 119 | 14.587 | −32.354 | 14.028 | 1.00 | 21.33 | N |
| ATOM | 994 | CA | PRO | H | 119 | 14.276 | −31.183 | 14.855 | 1.00 | 20.90 | C |
| ATOM | 995 | CB | PRO | H | 119 | 15.227 | −30.120 | 14.324 | 1.00 | 20.74 | C |
| ATOM | 996 | CG | PRO | H | 119 | 15.427 | −30.484 | 12.885 | 1.00 | 21.22 | C |
| ATOM | 997 | CD | PRO | H | 119 | 15.372 | −31.993 | 12.831 | 1.00 | 21.17 | C |
| ATOM | 998 | C | PRO | H | 119 | 12.851 | −30.663 | 14.720 | 1.00 | 20.87 | C |
| ATOM | 999 | O | PRO | H | 119 | 12.194 | −30.865 | 13.692 | 1.00 | 20.48 | O |
| ATOM | 1000 | N | SER | H | 120 | 12.382 | −29.991 | 15.769 | 1.00 | 20.31 | N |
| ATOM | 1001 | CA | SER | H | 120 | 11.280 | −29.055 | 15.632 | 1.00 | 20.08 | C |
| ATOM | 1002 | CB | SER | H | 120 | 10.467 | −28.986 | 16.921 | 1.00 | 20.18 | C |
| ATOM | 1003 | OG | SER | H | 120 | 9.822 | −30.217 | 17.154 | 1.00 | 21.68 | O |
| ATOM | 1004 | C | SER | H | 120 | 11.866 | −27.684 | 15.319 | 1.00 | 19.48 | C |
| ATOM | 1005 | O | SER | H | 120 | 12.963 | −27.344 | 15.771 | 1.00 | 19.57 | O |
| ATOM | 1006 | N | VAL | H | 121 | 11.146 | −26.896 | 14.534 | 1.00 | 18.69 | N |
| ATOM | 1007 | CA | VAL | H | 121 | 11.646 | −25.589 | 14.133 | 1.00 | 18.35 | C |
| ATOM | 1008 | CB | VAL | H | 121 | 11.949 | −25.542 | 12.602 | 1.00 | 18.43 | C |
| ATOM | 1009 | CG1 | VAL | H | 121 | 12.459 | −24.171 | 12.182 | 1.00 | 18.06 | C |
| ATOM | 1010 | CG2 | VAL | H | 121 | 12.970 | −26.632 | 12.233 | 1.00 | 18.17 | C |
| ATOM | 1011 | C | VAL | H | 121 | 10.645 | −24.521 | 14.544 | 1.00 | 18.30 | C |
| ATOM | 1012 | O | VAL | H | 121 | 9.489 | −24.543 | 14.113 | 1.00 | 18.06 | O |
| ATOM | 1013 | N | PHE | H | 122 | 11.082 | −23.593 | 15.389 | 1.00 | 17.62 | N |
| ATOM | 1014 | CA | PHE | H | 122 | 10.199 | −22.526 | 15.857 | 1.00 | 17.20 | C |
| ATOM | 1015 | CB | PHE | H | 122 | 10.032 | −22.593 | 17.388 | 1.00 | 17.29 | C |
| ATOM | 1016 | CG | PHE | H | 122 | 9.616 | −23.944 | 17.894 | 1.00 | 17.29 | C |
| ATOM | 1017 | CD1 | PHE | H | 122 | 8.337 | −24.449 | 17.621 | 1.00 | 16.35 | C |
| ATOM | 1018 | CE1 | PHE | H | 122 | 7.948 | −25.698 | 18.082 | 1.00 | 15.96 | C |
| ATOM | 1019 | CZ | PHE | H | 122 | 8.831 | −26.456 | 18.845 | 1.00 | 16.17 | C |
| ATOM | 1020 | CE2 | PHE | H | 122 | 10.115 | −25.965 | 19.127 | 1.00 | 17.06 | C |
| ATOM | 1021 | CD2 | PHE | H | 122 | 10.495 | −24.711 | 18.652 | 1.00 | 16.50 | C |
| ATOM | 1022 | C | PHE | H | 122 | 10.706 | −21.144 | 15.449 | 1.00 | 16.96 | C |
| ATOM | 1023 | O | PHE | H | 122 | 11.907 | −20.899 | 15.469 | 1.00 | 17.36 | O |
| ATOM | 1024 | N | PRO | H | 123 | 9.791 | −20.227 | 15.094 | 1.00 | 16.91 | N |
| ATOM | 1025 | CA | PRO | H | 123 | 10.226 | −18.893 | 14.695 | 1.00 | 17.00 | C |
| ATOM | 1026 | CB | PRO | H | 123 | 8.960 | −18.291 | 14.082 | 1.00 | 17.35 | C |
| ATOM | 1027 | CG | PRO | H | 123 | 7.839 | −18.930 | 14.882 | 1.00 | 17.35 | C |
| ATOM | 1028 | CD | PRO | H | 123 | 8.318 | −20.328 | 15.194 | 1.00 | 16.97 | C |
| ATOM | 1029 | C | PRO | H | 123 | 10.719 | −18.038 | 15.879 | 1.00 | 17.57 | C |
| ATOM | 1030 | O | PRO | H | 123 | 10.193 | −18.140 | 16.997 | 1.00 | 16.49 | O |
| ATOM | 1031 | N | LEU | H | 124 | 11.754 | −17.242 | 15.629 | 1.00 | 17.57 | N |
| ATOM | 1032 | CA | LEU | H | 124 | 12.172 | −16.191 | 16.553 | 1.00 | 18.40 | C |
| ATOM | 1033 | CB | LEU | H | 124 | 13.696 | −16.226 | 16.803 | 1.00 | 17.85 | C |
| ATOM | 1034 | CG | LEU | H | 124 | 14.297 | −17.451 | 17.523 | 1.00 | 18.59 | C |
| ATOM | 1035 | CD1 | LEU | H | 124 | 15.852 | −17.529 | 17.441 | 1.00 | 17.52 | C |
| ATOM | 1036 | CD2 | LEU | H | 124 | 13.853 | −17.541 | 18.973 | 1.00 | 18.03 | C |
| ATOM | 1037 | C | LEU | H | 124 | 11.731 | −14.890 | 15.894 | 1.00 | 18.67 | C |
| ATOM | 1038 | O | LEU | H | 124 | 12.463 | −14.306 | 15.095 | 1.00 | 18.06 | O |
| ATOM | 1039 | N | ALA | H | 125 | 10.514 | −14.454 | 16.217 | 1.00 | 19.55 | N |
| ATOM | 1040 | CA | ALA | H | 125 | 9.861 | −13.367 | 15.480 | 1.00 | 20.54 | C |
| ATOM | 1041 | CB | ALA | H | 125 | 8.354 | −13.370 | 15.750 | 1.00 | 20.95 | C |
| ATOM | 1042 | C | ALA | H | 125 | 10.467 | −12.000 | 15.802 | 1.00 | 21.55 | C |
| ATOM | 1043 | O | ALA | H | 125 | 10.814 | −11.740 | 16.955 | 1.00 | 21.15 | O |
| ATOM | 1044 | N | PRO | H | 126 | 10.602 | −11.123 | 14.785 | 1.00 | 22.39 | N |
| ATOM | 1045 | CA | PRO | H | 126 | 11.112 | −9.771 | 15.015 | 1.00 | 23.91 | C |
| ATOM | 1046 | CB | PRO | H | 126 | 11.374 | −9.263 | 13.596 | 1.00 | 24.12 | C |
| ATOM | 1047 | CG | PRO | H | 126 | 10.361 | −9.977 | 12.759 | 1.00 | 22.54 | C |
| ATOM | 1048 | CD | PRO | H | 126 | 10.266 | −11.344 | 13.361 | 1.00 | 22.74 | C |
| ATOM | 1049 | C | PRO | H | 126 | 10.068 | −8.875 | 15.687 | 1.00 | 25.76 | C |
| ATOM | 1050 | O | PRO | H | 126 | 8.869 | −9.137 | 15.566 | 1.00 | 25.94 | O |
| ATOM | 1051 | N | SER | H | 127 | 10.527 | −7.829 | 16.377 | 1.00 | 27.54 | N |
| ATOM | 1052 | CA | SER | H | 127 | 9.648 | −6.779 | 16.915 | 1.00 | 29.34 | C |
| ATOM | 1053 | CB | SER | H | 127 | 8.870 | −7.292 | 18.135 | 1.00 | 29.46 | C |
| ATOM | 1054 | OG | SER | H | 127 | 9.757 | −7.830 | 19.108 | 1.00 | 29.92 | O |
| ATOM | 1055 | C | SER | H | 127 | 10.451 | −5.547 | 17.326 | 1.00 | 30.23 | C |
| ATOM | 1056 | O | SER | H | 127 | 11.570 | −5.675 | 17.829 | 1.00 | 30.79 | O |
| ATOM | 1057 | N | SER | H | 128 | 9.879 | −4.360 | 17.120 | 1.00 | 31.20 | N |
| ATOM | 1058 | CA | SER | H | 128 | 10.453 | −3.127 | 17.677 | 1.00 | 31.65 | C |
| ATOM | 1059 | CB | SER | H | 128 | 11.065 | −2.238 | 16.581 | 1.00 | 31.65 | C |
| ATOM | 1060 | OG | SER | H | 128 | 10.212 | −1.150 | 16.253 | 1.00 | 32.20 | O |
| ATOM | 1061 | C | SER | H | 128 | 9.401 | −2.363 | 18.485 | 1.00 | 31.65 | C |
| ATOM | 1062 | O | SER | H | 128 | 9.072 | −2.739 | 19.613 | 1.00 | 31.79 | O |
| ATOM | 1063 | N | GLY | H | 133 | 12.983 | 3.633 | 11.775 | 1.00 | 31.43 | N |
| ATOM | 1064 | CA | GLY | H | 133 | 13.424 | 2.545 | 10.919 | 1.00 | 31.20 | C |
| ATOM | 1065 | C | GLY | H | 133 | 14.927 | 2.350 | 10.996 | 1.00 | 31.36 | C |
| ATOM | 1066 | O | GLY | H | 133 | 15.693 | 3.188 | 10.509 | 1.00 | 31.62 | O |
| ATOM | 1067 | N | GLY | H | 134 | 15.346 | 1.245 | 11.621 | 1.00 | 30.99 | N |
| ATOM | 1068 | CA | GLY | H | 134 | 16.749 | 0.836 | 11.681 | 1.00 | 30.10 | C |
| ATOM | 1069 | C | GLY | H | 134 | 16.888 | −0.608 | 11.225 | 1.00 | 29.64 | C |
| ATOM | 1070 | O | GLY | H | 134 | 16.474 | −0.954 | 10.114 | 1.00 | 29.76 | O |
| ATOM | 1071 | N | THR | H | 135 | 17.448 | −1.455 | 12.087 | 1.00 | 28.83 | N |
| ATOM | 1072 | CA | THR | H | 135 | 17.615 | −2.881 | 11.770 | 1.00 | 28.10 | C |
| ATOM | 1073 | CB | THR | H | 135 | 19.097 | −3.274 | 11.513 | 1.00 | 28.45 | C |
| ATOM | 1074 | OG1 | THR | H | 135 | 19.944 | −2.605 | 12.452 | 1.00 | 29.62 | O |
| ATOM | 1075 | CG2 | THR | H | 135 | 19.532 | −2.907 | 10.086 | 1.00 | 28.64 | C |
| ATOM | 1076 | C | THR | H | 135 | 17.022 | −3.805 | 12.832 | 1.00 | 26.89 | C |
| ATOM | 1077 | O | THR | H | 135 | 17.296 | −3.652 | 14.028 | 1.00 | 27.21 | O |
| ATOM | 1078 | N | ALA | H | 136 | 16.220 | −4.766 | 12.372 | 1.00 | 24.94 | N |
| ATOM | 1079 | CA | ALA | H | 136 | 15.579 | −5.756 | 13.228 | 1.00 | 22.89 | C |
| ATOM | 1080 | CB | ALA | H | 136 | 14.124 | −5.887 | 12.855 | 1.00 | 23.19 | C |
| ATOM | 1081 | C | ALA | H | 136 | 16.255 | −7.122 | 13.157 | 1.00 | 21.75 | C |
| ATOM | 1082 | O | ALA | H | 136 | 16.787 | −7.513 | 12.113 | 1.00 | 21.43 | O |
| ATOM | 1083 | N | ALA | H | 137 | 16.214 | −7.853 | 14.266 | 1.00 | 19.84 | N |
| ATOM | 1084 | CA | ALA | H | 137 | 16.727 | −9.227 | 14.293 | 1.00 | 18.86 | C |
| ATOM | 1085 | CB | ALA | H | 137 | 17.654 | −9.438 | 15.492 | 1.00 | 18.48 | C |
| ATOM | 1086 | C | ALA | H | 137 | 15.584 | −10.245 | 14.315 | 1.00 | 17.83 | C |
| ATOM | 1087 | O | ALA | H | 137 | 14.555 | −10.021 | 14.941 | 1.00 | 17.47 | O |
| ATOM | 1088 | N | LEU | H | 138 | 15.785 | −11.359 | 13.623 | 1.00 | 17.11 | N |
| ATOM | 1089 | CA | LEU | H | 138 | 14.847 | −12.473 | 13.631 | 1.00 | 16.59 | C |
| ATOM | 1090 | CB | LEU | H | 138 | 13.785 | −12.291 | 12.536 | 1.00 | 17.07 | C |
| ATOM | 1091 | CG | LEU | H | 138 | 14.252 | −12.161 | 11.085 | 1.00 | 17.24 | C |
| ATOM | 1092 | CD1 | LEU | H | 138 | 14.350 | −13.537 | 10.460 | 1.00 | 18.92 | C |
| ATOM | 1093 | CD2 | LEU | H | 138 | 13.280 | −11.315 | 10.304 | 1.00 | 18.91 | C |
| ATOM | 1094 | C | LEU | H | 138 | 15.602 | −13.787 | 13.460 | 1.00 | 16.67 | C |
| ATOM | 1095 | O | LEU | H | 138 | 16.812 | −13.791 | 13.206 | 1.00 | 16.20 | O |
| ATOM | 1096 | N | GLY | H | 139 | 14.900 | −14.906 | 13.601 | 1.00 | 16.45 | N |
| ATOM | 1097 | CA | GLY | H | 139 | 15.568 | −16.190 | 13.499 | 1.00 | 16.49 | C |
| ATOM | 1098 | C | GLY | H | 139 | 14.683 | −17.401 | 13.603 | 1.00 | 16.91 | C |
| ATOM | 1099 | O | GLY | H | 139 | 13.461 | −17.298 | 13.527 | 1.00 | 16.60 | O |
| ATOM | 1100 | N | CYS | H | 140 | 15.333 | −18.550 | 13.744 | 1.00 | 17.75 | N |
| ATOM | 1101 | CA | CYS | H | 140 | 14.685 | −19.830 | 13.952 | 1.00 | 18.81 | C |
| ATOM | 1102 | CB | CYS | H | 140 | 14.677 | −20.652 | 12.672 | 1.00 | 19.53 | C |
| ATOM | 1103 | SG | CYS | H | 140 | 13.493 | −20.090 | 11.449 | 1.00 | 24.27 | S |
| ATOM | 1104 | C | CYS | H | 140 | 15.403 | −20.622 | 15.027 | 1.00 | 18.50 | C |
| ATOM | 1105 | O | CYS | H | 140 | 16.634 | −20.725 | 15.034 | 1.00 | 18.34 | O |
| ATOM | 1106 | N | LEU | H | 141 | 14.623 | −21.161 | 15.954 | 1.00 | 17.67 | N |
| ATOM | 1107 | CA | LEU | H | 141 | 15.149 | −22.052 | 16.966 | 1.00 | 17.80 | C |
| ATOM | 1108 | CB | LEU | H | 141 | 14.391 | −21.877 | 18.290 | 1.00 | 17.16 | C |
| ATOM | 1109 | CG | LEU | H | 141 | 14.677 | −22.859 | 19.429 | 1.00 | 17.13 | C |
| ATOM | 1110 | CD1 | LEU | H | 141 | 16.119 | −22.840 | 19.871 | 1.00 | 15.55 | C |
| ATOM | 1111 | CD2 | LEU | H | 141 | 13.754 | −22.578 | 20.618 | 1.00 | 14.67 | C |
| ATOM | 1112 | C | LEU | H | 141 | 14.960 | −23.461 | 16.432 | 1.00 | 18.01 | C |
| ATOM | 1113 | O | LEU | H | 141 | 13.853 | −23.854 | 16.059 | 1.00 | 17.99 | O |
| ATOM | 1114 | N | VAL | H | 142 | 16.049 | −24.209 | 16.381 | 1.00 | 18.19 | N |
| ATOM | 1115 | CA | VAL | H | 142 | 16.041 | −25.556 | 15.829 | 1.00 | 18.39 | C |
| ATOM | 1116 | CB | VAL | H | 142 | 17.136 | −25.718 | 14.743 | 1.00 | 17.91 | C |
| ATOM | 1117 | CG1 | VAL | H | 142 | 17.039 | −27.085 | 14.093 | 1.00 | 17.26 | C |
| ATOM | 1118 | CG2 | VAL | H | 142 | 16.992 | −24.639 | 13.707 | 1.00 | 17.06 | C |
| ATOM | 1119 | C | VAL | H | 142 | 16.289 | −26.475 | 16.996 | 1.00 | 18.87 | C |
| ATOM | 1120 | O | VAL | H | 142 | 17.409 | −26.579 | 17.502 | 1.00 | 19.87 | O |
| ATOM | 1121 | N | LYS | H | 143 | 15.225 | −27.109 | 17.467 | 1.00 | 19.61 | N |
| ATOM | 1122 | CA | LYS | H | 143 | 15.277 | −27.749 | 18.779 | 1.00 | 19.34 | C |
| ATOM | 1123 | CB | LYS | H | 143 | 14.260 | −27.092 | 19.727 | 1.00 | 19.76 | C |
| ATOM | 1124 | CG | LYS | H | 143 | 14.466 | −27.419 | 21.203 | 1.00 | 21.31 | C |
| ATOM | 1125 | CD | LYS | H | 143 | 13.458 | −26.683 | 22.082 | 1.00 | 21.93 | C |
| ATOM | 1126 | CE | LYS | H | 143 | 13.301 | −27.322 | 23.450 | 1.00 | 22.67 | C |
| ATOM | 1127 | NZ | LYS | H | 143 | 14.585 | −27.504 | 24.154 | 1.00 | 24.21 | N |
| ATOM | 1128 | C | LYS | H | 143 | 15.066 | −29.253 | 18.709 | 1.00 | 19.16 | C |
| ATOM | 1129 | O | LYS | H | 143 | 14.257 | −29.756 | 17.904 | 1.00 | 18.73 | O |
| ATOM | 1130 | N | ASP | H | 144 | 15.822 | −29.956 | 19.550 | 1.00 | 18.87 | N |
| ATOM | 1131 | CA | ASP | H | 144 | 15.565 | −31.347 | 19.897 | 1.00 | 18.59 | C |
| ATOM | 1132 | CB | ASP | H | 144 | 14.159 | −31.511 | 20.492 | 1.00 | 18.68 | C |
| ATOM | 1133 | CG | ASP | H | 144 | 14.074 | −31.015 | 21.938 | 1.00 | 18.79 | C |
| ATOM | 1134 | OD1 | ASP | H | 144 | 15.129 | −30.738 | 22.534 | 1.00 | 18.11 | O |
| ATOM | 1135 | OD2 | ASP | H | 144 | 12.953 | −30.891 | 22.465 | 1.00 | 20.32 | O |
| ATOM | 1136 | C | ASP | H | 144 | 15.783 | −32.277 | 18.719 | 1.00 | 18.86 | C |
| ATOM | 1137 | O | ASP | H | 144 | 14.856 | −32.952 | 18.259 | 1.00 | 19.27 | O |
| ATOM | 1138 | N | TYR | H | 145 | 17.021 | −32.306 | 18.236 | 1.00 | 18.48 | N |
| ATOM | 1139 | CA | TYR | H | 145 | 17.421 | −33.261 | 17.224 | 1.00 | 18.78 | C |
| ATOM | 1140 | CB | TYR | H | 145 | 17.690 | −32.555 | 15.897 | 1.00 | 18.55 | C |
| ATOM | 1141 | CG | TYR | H | 145 | 18.871 | −31.625 | 15.926 | 1.00 | 18.48 | C |
| ATOM | 1142 | CD1 | TYR | H | 145 | 18.717 | −30.299 | 16.326 | 1.00 | 18.99 | C |
| ATOM | 1143 | CE1 | TYR | H | 145 | 19.799 | −29.426 | 16.356 | 1.00 | 19.47 | C |
| ATOM | 1144 | CZ | TYR | H | 145 | 21.054 | −29.867 | 15.958 | 1.00 | 20.37 | C |
| ATOM | 1145 | OH | TYR | H | 145 | 22.100 | −28.969 | 15.986 | 1.00 | 20.25 | O |
| ATOM | 1146 | CE2 | TYR | H | 145 | 21.247 | −31.183 | 15.549 | 1.00 | 18.72 | C |
| ATOM | 1147 | CD2 | TYR | H | 145 | 20.145 | −32.061 | 15.527 | 1.00 | 18.50 | C |
| ATOM | 1148 | C | TYR | H | 145 | 18.647 | −34.069 | 17.660 | 1.00 | 19.25 | C |
| ATOM | 1149 | O | TYR | H | 145 | 19.397 | −33.657 | 18.562 | 1.00 | 19.00 | O |
| ATOM | 1150 | N | PHE | H | 146 | 18.825 | −35.217 | 17.003 | 1.00 | 19.63 | N |
| ATOM | 1151 | CA | PHE | H | 146 | 19.966 | −36.106 | 17.185 | 1.00 | 20.15 | C |
| ATOM | 1152 | CB | PHE | H | 146 | 19.793 | −36.975 | 18.445 | 1.00 | 20.16 | C |
| ATOM | 1153 | CG | PHE | H | 146 | 20.957 | −37.899 | 18.711 | 1.00 | 20.10 | C |
| ATOM | 1154 | CD1 | PHE | H | 146 | 20.980 | −39.196 | 18.181 | 1.00 | 20.52 | C |
| ATOM | 1155 | CE1 | PHE | H | 146 | 22.079 | −40.054 | 18.409 | 1.00 | 20.69 | C |
| ATOM | 1156 | CZ | PHE | H | 146 | 23.150 | −39.616 | 19.177 | 1.00 | 20.00 | C |
| ATOM | 1157 | CE2 | PHE | H | 146 | 23.134 | −38.322 | 19.719 | 1.00 | 20.27 | C |
| ATOM | 1158 | CD2 | PHE | H | 146 | 22.034 | −37.473 | 19.480 | 1.00 | 20.58 | C |
| ATOM | 1159 | C | PHE | H | 146 | 20.101 | −37.001 | 15.941 | 1.00 | 21.00 | C |
| ATOM | 1160 | O | PHE | H | 146 | 19.090 | −37.481 | 15.413 | 1.00 | 20.13 | O |
| ATOM | 1161 | N | PRO | H | 147 | 21.342 | −37.226 | 15.463 | 1.00 | 21.80 | N |
| ATOM | 1162 | CA | PRO | H | 147 | 22.599 | −36.622 | 15.910 | 1.00 | 22.67 | C |
| ATOM | 1163 | CB | PRO | H | 147 | 23.640 | −37.681 | 15.516 | 1.00 | 22.87 | C |
| ATOM | 1164 | CG | PRO | H | 147 | 23.068 | −38.278 | 14.258 | 1.00 | 22.43 | C |
| ATOM | 1165 | CD | PRO | H | 147 | 21.572 | −38.290 | 14.462 | 1.00 | 22.20 | C |
| ATOM | 1166 | C | PRO | H | 147 | 22.910 | −35.315 | 15.190 | 1.00 | 23.22 | C |
| ATOM | 1167 | O | PRO | H | 147 | 22.126 | −34.865 | 14.357 | 1.00 | 23.35 | O |
| ATOM | 1168 | N | GLU | H | 148 | 24.055 | −34.722 | 15.508 | 1.00 | 23.56 | N |
| ATOM | 1169 | CA | GLU | H | 148 | 24.604 | −33.637 | 14.693 | 1.00 | 25.21 | C |
| ATOM | 1170 | CB | GLU | H | 148 | 25.877 | −33.101 | 15.350 | 1.00 | 25.39 | C |
| ATOM | 1171 | CG | GLU | H | 148 | 25.590 | −32.074 | 16.434 | 1.00 | 28.36 | C |
| ATOM | 1172 | CD | GLU | H | 148 | 25.587 | −30.640 | 15.892 | 1.00 | 32.77 | C |
| ATOM | 1173 | OE1 | GLU | H | 148 | 24.686 | −30.267 | 15.082 | 1.00 | 34.09 | O |
| ATOM | 1174 | OE2 | GLU | H | 148 | 26.493 | −29.883 | 16.291 | 1.00 | 32.17 | O |
| ATOM | 1175 | C | GLU | H | 148 | 24.898 | −34.175 | 13.282 | 1.00 | 25.10 | C |
| ATOM | 1176 | O | GLU | H | 148 | 25.160 | −35.361 | 13.137 | 1.00 | 25.32 | O |
| ATOM | 1177 | N | PRO | H | 149 | 24.850 | −33.322 | 12.242 | 1.00 | 25.56 | N |
| ATOM | 1178 | CA | PRO | H | 149 | 24.550 | −31.894 | 12.196 | 1.00 | 25.44 | C |
| ATOM | 1179 | CB | PRO | H | 149 | 25.642 | −31.353 | 11.259 | 1.00 | 26.07 | C |
| ATOM | 1180 | CG | PRO | H | 149 | 26.108 | −32.549 | 10.425 | 1.00 | 26.36 | C |
| ATOM | 1181 | CD | PRO | H | 149 | 25.415 | −33.783 | 10.958 | 1.00 | 26.46 | C |
| ATOM | 1182 | C | PRO | H | 149 | 23.188 | −31.568 | 11.593 | 1.00 | 25.22 | C |
| ATOM | 1183 | O | PRO | H | 149 | 22.521 | −32.452 | 11.052 | 1.00 | 24.77 | O |
| ATOM | 1184 | N | VAL | H | 150 | 22.786 | −30.305 | 11.710 | 1.00 | 24.77 | N |
| ATOM | 1185 | CA | VAL | H | 150 | 21.745 | −29.732 | 10.863 | 1.00 | 25.09 | C |
| ATOM | 1186 | CB | VAL | H | 150 | 20.571 | −29.106 | 11.661 | 1.00 | 25.26 | C |
| ATOM | 1187 | CG1 | VAL | H | 150 | 19.761 | −30.176 | 12.372 | 1.00 | 26.75 | C |
| ATOM | 1188 | CG2 | VAL | H | 150 | 21.071 | −28.046 | 12.643 | 1.00 | 24.96 | C |
| ATOM | 1189 | C | VAL | H | 150 | 22.350 | −28.614 | 10.028 | 1.00 | 24.77 | C |
| ATOM | 1190 | O | VAL | H | 150 | 23.353 | −28.017 | 10.406 | 1.00 | 24.66 | O |
| ATOM | 1191 | N | THR | H | 151 | 21.728 | −28.325 | 8.894 | 1.00 | 24.31 | N |
| ATOM | 1192 | CA | THR | H | 151 | 22.133 | −27.172 | 8.112 | 1.00 | 24.15 | C |
| ATOM | 1193 | CB | THR | H | 151 | 22.492 | −27.548 | 6.667 | 1.00 | 24.33 | C |
| ATOM | 1194 | OG1 | THR | H | 151 | 21.366 | −28.181 | 6.063 | 1.00 | 25.28 | O |
| ATOM | 1195 | CG2 | THR | H | 151 | 23.704 | −28.507 | 6.643 | 1.00 | 26.37 | C |
| ATOM | 1196 | C | THR | H | 151 | 20.986 | −26.188 | 8.123 | 1.00 | 23.02 | C |
| ATOM | 1197 | O | THR | H | 151 | 19.809 | −26.579 | 8.178 | 1.00 | 22.21 | O |
| ATOM | 1198 | N | VAL | H | 152 | 21.330 | −24.907 | 8.121 | 1.00 | 21.72 | N |
| ATOM | 1199 | CA | VAL | H | 152 | 20.318 | −23.865 | 8.071 | 1.00 | 20.88 | C |
| ATOM | 1200 | CB | VAL | H | 152 | 20.148 | −23.130 | 9.436 | 1.00 | 21.05 | C |
| ATOM | 1201 | CG1 | VAL | H | 152 | 19.041 | −22.110 | 9.341 | 1.00 | 18.61 | C |
| ATOM | 1202 | CG2 | VAL | H | 152 | 19.854 | −24.119 | 10.556 | 1.00 | 20.38 | C |
| ATOM | 1203 | C | VAL | H | 152 | 20.721 | −22.864 | 7.022 | 1.00 | 20.65 | C |
| ATOM | 1204 | O | VAL | H | 152 | 21.859 | −22.382 | 7.018 | 1.00 | 20.74 | O |
| ATOM | 1205 | N | SER | H | 153 | 19.790 | −22.566 | 6.130 | 1.00 | 20.55 | N |
| ATOM | 1206 | CA | SER | H | 153 | 19.972 | −21.493 | 5.180 | 1.00 | 20.38 | C |
| ATOM | 1207 | CB | SER | H | 153 | 20.055 | −22.058 | 3.760 | 1.00 | 20.67 | C |
| ATOM | 1208 | OG | SER | H | 153 | 18.893 | −22.808 | 3.469 | 1.00 | 21.85 | O |
| ATOM | 1209 | C | SER | H | 153 | 18.798 | −20.544 | 5.333 | 1.00 | 19.61 | C |
| ATOM | 1210 | O | SER | H | 153 | 17.811 | −20.871 | 6.006 | 1.00 | 20.11 | O |
| ATOM | 1211 | N | TRP | H | 154 | 18.916 | −19.355 | 4.755 | 1.00 | 18.90 | N |
| ATOM | 1212 | CA | TRP | H | 154 | 17.794 | −18.424 | 4.704 | 1.00 | 18.40 | C |
| ATOM | 1213 | CB | TRP | H | 154 | 18.117 | −17.119 | 5.435 | 1.00 | 18.09 | C |
| ATOM | 1214 | CG | TRP | H | 154 | 18.141 | −17.335 | 6.935 | 1.00 | 17.10 | C |
| ATOM | 1215 | CD1 | TRP | H | 154 | 19.207 | −17.729 | 7.694 | 1.00 | 17.84 | C |
| ATOM | 1216 | NE1 | TRP | H | 154 | 18.835 | −17.850 | 9.013 | 1.00 | 16.16 | N |
| ATOM | 1217 | CE2 | TRP | H | 154 | 17.502 | −17.548 | 9.122 | 1.00 | 16.93 | C |
| ATOM | 1218 | CD2 | TRP | H | 154 | 17.032 | −17.226 | 7.828 | 1.00 | 16.65 | C |
| ATOM | 1219 | CE3 | TRP | H | 154 | 15.685 | −16.882 | 7.662 | 1.00 | 17.04 | C |
| ATOM | 1220 | CZ3 | TRP | H | 154 | 14.849 | −16.858 | 8.796 | 1.00 | 16.69 | C |
| ATOM | 1221 | CH2 | TRP | H | 154 | 15.353 | −17.181 | 10.063 | 1.00 | 17.51 | C |
| ATOM | 1222 | CZ2 | TRP | H | 154 | 16.673 | −17.531 | 10.245 | 1.00 | 16.53 | C |
| ATOM | 1223 | C | TRP | H | 154 | 17.367 | −18.194 | 3.264 | 1.00 | 18.74 | C |
| ATOM | 1224 | O | TRP | H | 154 | 18.221 | −18.052 | 2.363 | 1.00 | 18.58 | O |
| ATOM | 1225 | N | ASN | H | 155 | 16.047 | −18.171 | 3.061 | 1.00 | 18.76 | N |
| ATOM | 1226 | CA | ASN | H | 155 | 15.436 | −17.995 | 1.735 | 1.00 | 19.10 | C |
| ATOM | 1227 | CB | ASN | H | 155 | 15.385 | −16.502 | 1.357 | 1.00 | 19.16 | C |
| ATOM | 1228 | CG | ASN | H | 155 | 14.521 | −15.689 | 2.317 | 1.00 | 19.89 | C |
| ATOM | 1229 | OD1 | ASN | H | 155 | 13.716 | −16.247 | 3.065 | 1.00 | 22.38 | O |
| ATOM | 1230 | ND2 | ASN | H | 155 | 14.671 | −14.373 | 2.288 | 1.00 | 20.09 | N |
| ATOM | 1231 | C | ASN | H | 155 | 16.130 | −18.830 | 0.660 | 1.00 | 19.03 | C |
| ATOM | 1232 | O | ASN | H | 155 | 16.520 | −18.313 | −0.391 | 1.00 | 19.22 | O |
| ATOM | 1233 | N | SER | H | 156 | 16.295 | −20.123 | 0.950 | 1.00 | 19.33 | N |
| ATOM | 1234 | CA | SER | H | 156 | 16.993 | −21.069 | 0.063 | 1.00 | 19.42 | C |
| ATOM | 1235 | CB | SER | H | 156 | 16.133 | −21.356 | −1.175 | 1.00 | 19.71 | C |
| ATOM | 1236 | OG | SER | H | 156 | 14.857 | −21.810 | −0.778 | 1.00 | 19.20 | O |
| ATOM | 1237 | C | SER | H | 156 | 18.407 | −20.611 | −0.340 | 1.00 | 19.86 | C |
| ATOM | 1238 | O | SER | H | 156 | 18.787 | −20.672 | −1.521 | 1.00 | 19.69 | O |
| ATOM | 1239 | N | GLY | H | 157 | 19.171 | −20.126 | 0.642 | 1.00 | 19.36 | N |
| ATOM | 1240 | CA | GLY | H | 157 | 20.541 | −19.648 | 0.401 | 1.00 | 19.35 | C |
| ATOM | 1241 | C | GLY | H | 157 | 20.671 | −18.288 | −0.289 | 1.00 | 19.12 | C |
| ATOM | 1242 | O | GLY | H | 157 | 21.789 | −17.836 | −0.563 | 1.00 | 19.06 | O |
| ATOM | 1243 | N | ALA | H | 158 | 19.546 | −17.631 | −0.561 | 1.00 | 18.79 | N |
| ATOM | 1244 | CA | ALA | H | 158 | 19.553 | −16.311 | −1.216 | 1.00 | 18.95 | C |
| ATOM | 1245 | CB | ALA | H | 158 | 18.178 | −15.984 | −1.810 | 1.00 | 19.09 | C |
| ATOM | 1246 | C | ALA | H | 158 | 20.001 | −15.195 | −0.265 | 1.00 | 18.98 | C |
| ATOM | 1247 | O | ALA | H | 158 | 20.465 | −14.143 | −0.714 | 1.00 | 19.20 | O |
| ATOM | 1248 | N | LEU | H | 159 | 19.825 | −15.425 | 1.037 | 1.00 | 18.32 | N |
| ATOM | 1249 | CA | LEU | H | 159 | 20.243 | −14.489 | 2.090 | 1.00 | 17.56 | C |
| ATOM | 1250 | CB | LEU | H | 159 | 19.032 | −14.088 | 2.950 | 1.00 | 17.28 | C |
| ATOM | 1251 | CG | LEU | H | 159 | 19.180 | −13.178 | 4.183 | 1.00 | 17.76 | C |
| ATOM | 1252 | CD1 | LEU | H | 159 | 19.590 | −11.760 | 3.808 | 1.00 | 18.46 | C |
| ATOM | 1253 | CD2 | LEU | H | 159 | 17.850 | −13.136 | 4.977 | 1.00 | 17.48 | C |
| ATOM | 1254 | C | LEU | H | 159 | 21.347 | −15.108 | 2.959 | 1.00 | 16.62 | C |
| ATOM | 1255 | O | LEU | H | 159 | 21.100 | −16.063 | 3.701 | 1.00 | 16.61 | O |
| ATOM | 1256 | N | THR | H | 160 | 22.560 | −14.565 | 2.851 | 1.00 | 16.10 | N |
| ATOM | 1257 | CA | THR | H | 160 | 23.694 | −14.968 | 3.695 | 1.00 | 15.92 | C |
| ATOM | 1258 | CB | THR | H | 160 | 24.914 | −15.465 | 2.860 | 1.00 | 16.06 | C |
| ATOM | 1259 | OG1 | THR | H | 160 | 25.322 | −14.433 | 1.949 | 1.00 | 14.51 | O |
| ATOM | 1260 | CG2 | THR | H | 160 | 24.581 | −16.745 | 2.101 | 1.00 | 16.68 | C |
| ATOM | 1261 | C | THR | H | 160 | 24.207 | −13.821 | 4.556 | 1.00 | 16.00 | C |
| ATOM | 1262 | O | THR | H | 160 | 24.755 | −14.047 | 5.637 | 1.00 | 15.07 | O |
| ATOM | 1263 | N | SER | H | 161 | 24.076 | −12.594 | 4.054 | 1.00 | 16.48 | N |
| ATOM | 1264 | CA | SER | H | 161 | 24.601 | −11.420 | 4.758 | 1.00 | 17.45 | C |
| ATOM | 1265 | CB | SER | H | 161 | 24.539 | −10.181 | 3.857 | 1.00 | 17.56 | C |
| ATOM | 1266 | OG | SER | H | 161 | 25.593 | −10.220 | 2.895 | 1.00 | 18.50 | O |
| ATOM | 1267 | C | SER | H | 161 | 23.866 | −11.165 | 6.084 | 1.00 | 17.54 | C |
| ATOM | 1268 | O | SER | H | 161 | 22.642 | −11.137 | 6.121 | 1.00 | 16.95 | O |
| ATOM | 1269 | N | GLY | H | 162 | 24.629 | −11.014 | 7.165 | 1.00 | 18.15 | N |
| ATOM | 1270 | CA | GLY | H | 162 | 24.051 | −10.701 | 8.481 | 1.00 | 18.57 | C |
| ATOM | 1271 | C | GLY | H | 162 | 23.427 | −11.889 | 9.200 | 1.00 | 18.51 | C |
| ATOM | 1272 | O | GLY | H | 162 | 22.738 | −11.713 | 10.206 | 1.00 | 19.05 | O |
| ATOM | 1273 | N | VAL | H | 163 | 23.649 | −13.094 | 8.672 | 1.00 | 18.08 | N |
| ATOM | 1274 | CA | VAL | H | 163 | 23.177 | −14.333 | 9.293 | 1.00 | 17.42 | C |
| ATOM | 1275 | CB | VAL | H | 163 | 22.930 | −15.433 | 8.224 | 1.00 | 18.06 | C |
| ATOM | 1276 | CG1 | VAL | H | 163 | 22.663 | −16.803 | 8.889 | 1.00 | 16.92 | C |
| ATOM | 1277 | CG2 | VAL | H | 163 | 21.791 | −15.027 | 7.264 | 1.00 | 17.09 | C |
| ATOM | 1278 | C | VAL | H | 163 | 24.204 | −14.863 | 10.287 | 1.00 | 17.50 | C |
| ATOM | 1279 | O | VAL | H | 163 | 25.398 | −14.870 | 10.003 | 1.00 | 16.66 | O |
| ATOM | 1280 | N | HIS | H | 164 | 23.738 | −15.328 | 11.445 | 1.00 | 16.96 | N |
| ATOM | 1281 | CA | HIS | H | 164 | 24.611 | −15.996 | 12.413 | 1.00 | 17.69 | C |
| ATOM | 1282 | CB | HIS | H | 164 | 24.811 | −15.138 | 13.665 | 1.00 | 17.44 | C |
| ATOM | 1283 | CG | HIS | H | 164 | 25.621 | −13.899 | 13.435 | 1.00 | 17.84 | C |
| ATOM | 1284 | ND1 | HIS | H | 164 | 26.950 | −13.930 | 13.064 | 1.00 | 17.31 | N |
| ATOM | 1285 | CE1 | HIS | H | 164 | 27.402 | −12.693 | 12.947 | 1.00 | 17.25 | C |
| ATOM | 1286 | NE2 | HIS | H | 164 | 26.419 | −11.861 | 13.248 | 1.00 | 17.09 | N |
| ATOM | 1287 | CD2 | HIS | H | 164 | 25.298 | −12.591 | 13.564 | 1.00 | 17.77 | C |
| ATOM | 1288 | C | HIS | H | 164 | 23.983 | −17.314 | 12.805 | 1.00 | 17.62 | C |
| ATOM | 1289 | O | HIS | H | 164 | 22.891 | −17.337 | 13.367 | 1.00 | 17.23 | O |
| ATOM | 1290 | N | THR | H | 165 | 24.659 | −18.417 | 12.504 | 1.00 | 18.06 | N |
| ATOM | 1291 | CA | THR | H | 165 | 24.139 | −19.720 | 12.880 | 1.00 | 18.40 | C |
| ATOM | 1292 | CB | THR | H | 165 | 23.950 | −20.664 | 11.670 | 1.00 | 18.87 | C |
| ATOM | 1293 | OG1 | THR | H | 165 | 22.979 | −20.098 | 10.775 | 1.00 | 18.87 | O |
| ATOM | 1294 | CG2 | THR | H | 165 | 23.461 | −22.053 | 12.121 | 1.00 | 19.56 | C |
| ATOM | 1295 | C | THR | H | 165 | 25.046 | −20.282 | 13.959 | 1.00 | 18.72 | C |
| ATOM | 1296 | O | THR | H | 165 | 26.218 | −20.575 | 13.717 | 1.00 | 17.75 | O |
| ATOM | 1297 | N | PHE | H | 166 | 24.495 | −20.377 | 15.164 | 1.00 | 18.25 | N |
| ATOM | 1298 | CA | PHE | H | 166 | 25.269 | −20.710 | 16.344 | 1.00 | 18.65 | C |
| ATOM | 1299 | CB | PHE | H | 166 | 24.509 | −20.279 | 17.607 | 1.00 | 18.38 | C |
| ATOM | 1300 | CG | PHE | H | 166 | 24.398 | −18.798 | 17.749 | 1.00 | 17.26 | C |
| ATOM | 1301 | CD1 | PHE | H | 166 | 25.415 | −18.070 | 18.359 | 1.00 | 15.53 | C |
| ATOM | 1302 | CE1 | PHE | H | 166 | 25.319 | −16.692 | 18.477 | 1.00 | 15.35 | C |
| ATOM | 1303 | CZ | PHE | H | 166 | 24.207 | −16.017 | 17.954 | 1.00 | 16.65 | C |
| ATOM | 1304 | CE2 | PHE | H | 166 | 23.190 | −16.731 | 17.338 | 1.00 | 15.77 | C |
| ATOM | 1305 | CD2 | PHE | H | 166 | 23.295 | −18.113 | 17.225 | 1.00 | 16.56 | C |
| ATOM | 1306 | C | PHE | H | 166 | 25.597 | −22.192 | 16.390 | 1.00 | 19.58 | C |
| ATOM | 1307 | O | PHE | H | 166 | 24.843 | −23.010 | 15.837 | 1.00 | 19.55 | O |
| ATOM | 1308 | N | PRO | H | 167 | 26.730 | −22.546 | 17.030 | 1.00 | 20.26 | N |
| ATOM | 1309 | CA | PRO | H | 167 | 27.007 | −23.949 | 17.328 | 1.00 | 20.29 | C |
| ATOM | 1310 | CB | PRO | H | 167 | 28.252 | −23.885 | 18.230 | 1.00 | 21.14 | C |
| ATOM | 1311 | CG | PRO | H | 167 | 28.898 | −22.568 | 17.924 | 1.00 | 20.95 | C |
| ATOM | 1312 | CD | PRO | H | 167 | 27.729 | −21.641 | 17.635 | 1.00 | 20.86 | C |
| ATOM | 1313 | C | PRO | H | 167 | 25.861 | −24.556 | 18.129 | 1.00 | 20.40 | C |
| ATOM | 1314 | O | PRO | H | 167 | 25.277 | −23.872 | 18.994 | 1.00 | 19.98 | O |
| ATOM | 1315 | N | ALA | H | 168 | 25.560 | −25.827 | 17.862 | 1.00 | 20.82 | N |
| ATOM | 1316 | CA | ALA | H | 168 | 24.572 | −26.588 | 18.652 | 1.00 | 21.36 | C |
| ATOM | 1317 | CB | ALA | H | 168 | 24.362 | −27.945 | 18.033 | 1.00 | 22.02 | C |
| ATOM | 1318 | C | ALA | H | 168 | 25.054 | −26.770 | 20.074 | 1.00 | 21.49 | C |
| ATOM | 1319 | O | ALA | H | 168 | 26.261 | −26.848 | 20.302 | 1.00 | 21.58 | O |
| ATOM | 1320 | N | VAL | H | 169 | 24.115 | −26.849 | 21.021 | 1.00 | 21.64 | N |
| ATOM | 1321 | CA | VAL | H | 169 | 24.417 | −27.240 | 22.402 | 1.00 | 21.83 | C |
| ATOM | 1322 | CB | VAL | H | 169 | 23.954 | −26.178 | 23.462 | 1.00 | 22.60 | C |
| ATOM | 1323 | CG1 | VAL | H | 169 | 24.776 | −24.913 | 23.354 | 1.00 | 23.61 | C |
| ATOM | 1324 | CG2 | VAL | H | 169 | 22.468 | −25.853 | 23.327 | 1.00 | 22.24 | C |
| ATOM | 1325 | C | VAL | H | 169 | 23.718 | −28.571 | 22.683 | 1.00 | 21.86 | C |
| ATOM | 1326 | O | VAL | H | 169 | 22.611 | −28.807 | 22.206 | 1.00 | 21.66 | O |
| ATOM | 1327 | N | LEU | H | 170 | 24.368 | −29.443 | 23.438 | 1.00 | 21.50 | N |
| ATOM | 1328 | CA | LEU | H | 170 | 23.753 | −30.708 | 23.813 | 1.00 | 22.03 | C |
| ATOM | 1329 | CB | LEU | H | 170 | 24.830 | −31.792 | 23.973 | 1.00 | 21.91 | C |
| ATOM | 1330 | CG | LEU | H | 170 | 24.429 | −33.139 | 24.586 | 1.00 | 22.85 | C |
| ATOM | 1331 | CD1 | LEU | H | 170 | 23.643 | −33.963 | 23.569 | 1.00 | 23.13 | C |
| ATOM | 1332 | CD2 | LEU | H | 170 | 25.686 | −33.882 | 25.059 | 1.00 | 24.27 | C |
| ATOM | 1333 | C | LEU | H | 170 | 22.955 | −30.520 | 25.108 | 1.00 | 21.88 | C |
| ATOM | 1334 | O | LEU | H | 170 | 23.518 | −30.147 | 26.126 | 1.00 | 22.27 | O |
| ATOM | 1335 | N | GLN | H | 171 | 21.650 | −30.760 | 25.062 | 1.00 | 21.71 | N |
| ATOM | 1336 | CA | GLN | H | 171 | 20.801 | −30.596 | 26.248 | 1.00 | 22.47 | C |
| ATOM | 1337 | CB | GLN | H | 171 | 19.360 | −30.274 | 25.842 | 1.00 | 22.92 | C |
| ATOM | 1338 | CG | GLN | H | 171 | 19.255 | −29.081 | 24.889 | 1.00 | 24.53 | C |
| ATOM | 1339 | CD | GLN | H | 171 | 17.965 | −29.068 | 24.094 | 1.00 | 26.25 | C |
| ATOM | 1340 | OE1 | GLN | H | 171 | 17.070 | −28.271 | 24.359 | 1.00 | 28.06 | O |
| ATOM | 1341 | NE2 | GLN | H | 171 | 17.864 | −29.954 | 23.109 | 1.00 | 27.44 | N |
| ATOM | 1342 | C | GLN | H | 171 | 20.867 | −31.830 | 27.163 | 1.00 | 22.12 | C |
| ATOM | 1343 | O | GLN | H | 171 | 21.382 | −32.879 | 26.763 | 1.00 | 21.74 | O |
| ATOM | 1344 | N | SER | H | 172 | 20.353 | −31.703 | 28.384 | 1.00 | 22.36 | N |
| ATOM | 1345 | CA | SER | H | 172 | 20.367 | −32.808 | 29.349 | 1.00 | 22.46 | C |
| ATOM | 1346 | CB | SER | H | 172 | 19.924 | −32.335 | 30.731 | 1.00 | 22.28 | C |
| ATOM | 1347 | OG | SER | H | 172 | 18.540 | −32.072 | 30.757 | 1.00 | 23.13 | O |
| ATOM | 1348 | C | SER | H | 172 | 19.521 | −34.006 | 28.889 | 1.00 | 22.57 | C |
| ATOM | 1349 | O | SER | H | 172 | 19.728 | −35.134 | 29.353 | 1.00 | 22.64 | O |
| ATOM | 1350 | N | SER | H | 173 | 18.592 | −33.757 | 27.965 | 1.00 | 21.95 | N |
| ATOM | 1351 | CA | SER | H | 173 | 17.814 | −34.813 | 27.314 | 1.00 | 21.77 | C |
| ATOM | 1352 | CB | SER | H | 173 | 16.654 | −34.183 | 26.541 | 1.00 | 22.14 | C |
| ATOM | 1353 | OG | SER | H | 173 | 17.149 | −33.460 | 25.417 | 1.00 | 20.89 | O |
| ATOM | 1354 | C | SER | H | 173 | 18.649 | −35.677 | 26.344 | 1.00 | 21.85 | C |
| ATOM | 1355 | O | SER | H | 173 | 18.191 | −36.726 | 25.883 | 1.00 | 22.14 | O |
| ATOM | 1356 | N | GLY | H | 174 | 19.863 | −35.234 | 26.023 | 1.00 | 20.80 | N |
| ATOM | 1357 | CA | GLY | H | 174 | 20.698 | −35.931 | 25.047 | 1.00 | 20.37 | C |
| ATOM | 1358 | C | GLY | H | 174 | 20.423 | −35.480 | 23.619 | 1.00 | 19.62 | C |
| ATOM | 1359 | O | GLY | H | 174 | 21.009 | −36.003 | 22.671 | 1.00 | 19.83 | O |
| ATOM | 1360 | N | LEU | H | 175 | 19.528 | −34.506 | 23.466 | 1.00 | 18.81 | N |
| ATOM | 1361 | CA | LEU | H | 175 | 19.217 | −33.940 | 22.155 | 1.00 | 18.81 | C |
| ATOM | 1362 | CB | LEU | H | 175 | 17.696 | −33.814 | 21.961 | 1.00 | 18.80 | C |
| ATOM | 1363 | CG | LEU | H | 175 | 16.829 | −35.062 | 22.181 | 1.00 | 19.08 | C |
| ATOM | 1364 | CD1 | LEU | H | 175 | 15.357 | −34.753 | 21.934 | 1.00 | 18.86 | C |
| ATOM | 1365 | CD2 | LEU | H | 175 | 17.293 | −36.216 | 21.282 | 1.00 | 19.62 | C |
| ATOM | 1366 | C | LEU | H | 175 | 19.886 | −32.574 | 21.974 | 1.00 | 18.53 | C |
| ATOM | 1367 | O | LEU | H | 175 | 20.081 | −31.834 | 22.937 | 1.00 | 18.15 | O |
| ATOM | 1368 | N | TYR | H | 176 | 20.223 | −32.246 | 20.734 | 1.00 | 18.53 | N |
| ATOM | 1369 | CA | TYR | H | 176 | 20.853 | −30.965 | 20.430 | 1.00 | 18.76 | C |
| ATOM | 1370 | CB | TYR | H | 176 | 21.802 | −31.109 | 19.236 | 1.00 | 18.13 | C |
| ATOM | 1371 | CG | TYR | H | 176 | 22.980 | −32.027 | 19.485 | 1.00 | 19.41 | C |
| ATOM | 1372 | CD1 | TYR | H | 176 | 24.134 | −31.562 | 20.122 | 1.00 | 21.22 | C |
| ATOM | 1373 | CE1 | TYR | H | 176 | 25.230 | −32.406 | 20.344 | 1.00 | 21.25 | C |
| ATOM | 1374 | CZ | TYR | H | 176 | 25.170 | −33.733 | 19.929 | 1.00 | 22.98 | C |
| ATOM | 1375 | OH | TYR | H | 176 | 26.237 | −34.591 | 20.134 | 1.00 | 21.67 | O |
| ATOM | 1376 | CE2 | TYR | H | 176 | 24.042 | −34.212 | 19.293 | 1.00 | 21.30 | C |
| ATOM | 1377 | CD2 | TYR | H | 176 | 22.948 | −33.362 | 19.083 | 1.00 | 20.65 | C |
| ATOM | 1378 | C | TYR | H | 176 | 19.854 | −29.851 | 20.166 | 1.00 | 18.92 | C |
| ATOM | 1379 | O | TYR | H | 176 | 18.696 | −30.099 | 19.798 | 1.00 | 18.70 | O |
| ATOM | 1380 | N | SER | H | 177 | 20.328 | −28.620 | 20.324 | 1.00 | 18.93 | N |
| ATOM | 1381 | CA | SER | H | 177 | 19.538 | −27.437 | 20.000 | 1.00 | 19.96 | C |
| ATOM | 1382 | CB | SER | H | 177 | 18.799 | −26.954 | 21.251 | 1.00 | 19.36 | C |
| ATOM | 1383 | OG | SER | H | 177 | 18.005 | −25.830 | 20.954 | 1.00 | 23.67 | O |
| ATOM | 1384 | C | SER | H | 177 | 20.443 | −26.326 | 19.454 | 1.00 | 19.73 | C |
| ATOM | 1385 | O | SER | H | 177 | 21.562 | −26.149 | 19.942 | 1.00 | 19.08 | O |
| ATOM | 1386 | N | LEU | H | 178 | 19.960 | −25.583 | 18.454 | 1.00 | 19.78 | N |
| ATOM | 1387 | CA | LEU | H | 178 | 20.667 | −24.386 | 17.978 | 1.00 | 19.93 | C |
| ATOM | 1388 | CB | LEU | H | 178 | 21.694 | −24.731 | 16.882 | 1.00 | 20.54 | C |
| ATOM | 1389 | CG | LEU | H | 178 | 21.339 | −25.119 | 15.439 | 1.00 | 20.29 | C |
| ATOM | 1390 | CD1 | LEU | H | 178 | 20.763 | −23.959 | 14.632 | 1.00 | 20.48 | C |
| ATOM | 1391 | CD2 | LEU | H | 178 | 22.597 | −25.643 | 14.741 | 1.00 | 20.65 | C |
| ATOM | 1392 | C | LEU | H | 178 | 19.720 | −23.300 | 17.494 | 1.00 | 19.90 | C |
| ATOM | 1393 | O | LEU | H | 178 | 18.577 | −23.579 | 17.136 | 1.00 | 20.25 | O |
| ATOM | 1394 | N | SER | H | 179 | 20.196 | −22.061 | 17.489 | 1.00 | 19.20 | N |
| ATOM | 1395 | CA | SER | H | 179 | 19.451 | −20.973 | 16.882 | 1.00 | 19.21 | C |
| ATOM | 1396 | CB | SER | H | 179 | 19.196 | −19.847 | 17.897 | 1.00 | 19.98 | C |
| ATOM | 1397 | OG | SER | H | 179 | 17.988 | −20.050 | 18.648 | 1.00 | 21.31 | O |
| ATOM | 1398 | C | SER | H | 179 | 20.206 | −20.433 | 15.664 | 1.00 | 18.76 | C |
| ATOM | 1399 | O | SER | H | 179 | 21.440 | −20.490 | 15.609 | 1.00 | 18.86 | O |
| ATOM | 1400 | N | SER | H | 180 | 19.457 | −19.917 | 14.695 | 1.00 | 18.21 | N |
| ATOM | 1401 | CA | SER | H | 180 | 20.033 | −19.186 | 13.577 | 1.00 | 17.96 | C |
| ATOM | 1402 | CB | SER | H | 180 | 19.891 | −19.955 | 12.261 | 1.00 | 17.99 | C |
| ATOM | 1403 | OG | SER | H | 180 | 20.424 | −19.191 | 11.188 | 1.00 | 17.71 | O |
| ATOM | 1404 | C | SER | H | 180 | 19.309 | −17.872 | 13.480 | 1.00 | 17.50 | C |
| ATOM | 1405 | O | SER | H | 180 | 18.070 | −17.831 | 13.455 | 1.00 | 18.04 | O |
| ATOM | 1406 | N | VAL | H | 181 | 20.081 | −16.796 | 13.428 | 1.00 | 16.70 | N |
| ATOM | 1407 | CA | VAL | H | 181 | 19.513 | −15.455 | 13.452 | 1.00 | 16.36 | C |
| ATOM | 1408 | CB | VAL | H | 181 | 19.798 | −14.703 | 14.798 | 1.00 | 16.30 | C |
| ATOM | 1409 | CG1 | VAL | H | 181 | 19.238 | −15.484 | 16.010 | 1.00 | 15.50 | C |
| ATOM | 1410 | CG2 | VAL | H | 181 | 21.282 | −14.420 | 14.981 | 1.00 | 15.61 | C |
| ATOM | 1411 | C | VAL | H | 181 | 20.029 | −14.647 | 12.264 | 1.00 | 17.05 | C |
| ATOM | 1412 | O | VAL | H | 181 | 21.066 | −14.962 | 11.691 | 1.00 | 16.94 | O |
| ATOM | 1413 | N | VAL | H | 182 | 19.279 | −13.621 | 11.892 | 1.00 | 17.90 | N |
| ATOM | 1414 | CA | VAL | H | 182 | 19.699 | −12.682 | 10.871 | 1.00 | 18.45 | C |
| ATOM | 1415 | CB | VAL | H | 182 | 19.185 | −13.109 | 9.458 | 1.00 | 18.33 | C |
| ATOM | 1416 | CG1 | VAL | H | 182 | 17.670 | −13.069 | 9.384 | 1.00 | 18.82 | C |
| ATOM | 1417 | CG2 | VAL | H | 182 | 19.788 | −12.233 | 8.353 | 1.00 | 18.95 | C |
| ATOM | 1418 | C | VAL | H | 182 | 19.214 | −11.283 | 11.263 | 1.00 | 18.57 | C |
| ATOM | 1419 | O | VAL | H | 182 | 18.156 | −11.132 | 11.867 | 1.00 | 18.95 | O |
| ATOM | 1420 | N | THR | H | 183 | 20.013 | −10.269 | 10.961 | 1.00 | 19.06 | N |
| ATOM | 1421 | CA | THR | H | 183 | 19.570 | −8.890 | 11.097 | 1.00 | 19.75 | C |
| ATOM | 1422 | CB | THR | H | 183 | 20.580 | −8.022 | 11.837 | 1.00 | 19.55 | C |
| ATOM | 1423 | OG1 | THR | H | 183 | 21.848 | −8.118 | 11.195 | 1.00 | 19.59 | O |
| ATOM | 1424 | CG2 | THR | H | 183 | 20.715 | −8.483 | 13.275 | 1.00 | 20.45 | C |
| ATOM | 1425 | C | THR | H | 183 | 19.266 | −8.292 | 9.721 | 1.00 | 20.31 | C |
| ATOM | 1426 | O | THR | H | 183 | 20.069 | −8.392 | 8.788 | 1.00 | 19.78 | O |
| ATOM | 1427 | N | VAL | H | 184 | 18.087 | −7.690 | 9.612 | 1.00 | 21.05 | N |
| ATOM | 1428 | CA | VAL | H | 184 | 17.578 | −7.163 | 8.352 | 1.00 | 22.15 | C |
| ATOM | 1429 | CB | VAL | H | 184 | 16.433 | −8.058 | 7.800 | 1.00 | 22.15 | C |
| ATOM | 1430 | CG1 | VAL | H | 184 | 16.922 | −9.491 | 7.597 | 1.00 | 21.58 | C |
| ATOM | 1431 | CG2 | VAL | H | 184 | 15.205 | −8.031 | 8.719 | 1.00 | 22.30 | C |
| ATOM | 1432 | C | VAL | H | 184 | 17.096 | −5.719 | 8.555 | 1.00 | 22.97 | C |
| ATOM | 1433 | O | VAL | H | 184 | 16.941 | −5.284 | 9.692 | 1.00 | 23.01 | O |
| ATOM | 1434 | N | PRO | H | 185 | 16.872 | −4.964 | 7.463 | 1.00 | 23.79 | N |
| ATOM | 1435 | CA | PRO | H | 185 | 16.293 | −3.634 | 7.686 | 1.00 | 24.71 | C |
| ATOM | 1436 | CB | PRO | H | 185 | 16.240 | −3.036 | 6.277 | 1.00 | 24.29 | C |
| ATOM | 1437 | CG | PRO | H | 185 | 17.346 | −3.729 | 5.549 | 1.00 | 24.53 | C |
| ATOM | 1438 | CD | PRO | H | 185 | 17.332 | −5.136 | 6.073 | 1.00 | 23.79 | C |
| ATOM | 1439 | C | PRO | H | 185 | 14.888 | −3.708 | 8.305 | 1.00 | 25.60 | C |
| ATOM | 1440 | O | PRO | H | 185 | 14.108 | −4.599 | 7.962 | 1.00 | 25.60 | O |
| ATOM | 1441 | N | SER | H | 186 | 14.586 | −2.791 | 9.224 | 1.00 | 26.83 | N |
| ATOM | 1442 | CA | SER | H | 186 | 13.258 | −2.716 | 9.836 | 1.00 | 28.32 | C |
| ATOM | 1443 | CB | SER | H | 186 | 13.195 | −1.592 | 10.879 | 1.00 | 28.49 | C |
| ATOM | 1444 | OG | SER | H | 186 | 14.068 | −1.838 | 11.969 | 1.00 | 28.66 | O |
| ATOM | 1445 | C | SER | H | 186 | 12.176 | −2.486 | 8.778 | 1.00 | 29.28 | C |
| ATOM | 1446 | O | SER | H | 186 | 11.131 | −3.138 | 8.791 | 1.00 | 29.46 | O |
| ATOM | 1447 | N | SER | H | 187 | 12.449 | −1.564 | 7.858 | 1.00 | 30.36 | N |
| ATOM | 1448 | CA | SER | H | 187 | 11.498 | −1.171 | 6.812 | 1.00 | 31.45 | C |
| ATOM | 1449 | CB | SER | H | 187 | 12.072 | −0.005 | 6.001 | 1.00 | 31.35 | C |
| ATOM | 1450 | OG | SER | H | 187 | 13.361 | −0.333 | 5.500 | 1.00 | 32.04 | O |
| ATOM | 1451 | C | SER | H | 187 | 11.094 | −2.318 | 5.870 | 1.00 | 32.00 | C |
| ATOM | 1452 | O | SER | H | 187 | 10.014 | −2.282 | 5.280 | 1.00 | 32.46 | O |
| ATOM | 1453 | N | SER | H | 188 | 11.957 | −3.322 | 5.730 | 1.00 | 32.68 | N |
| ATOM | 1454 | CA | SER | H | 188 | 11.665 | −4.476 | 4.875 | 1.00 | 33.19 | C |
| ATOM | 1455 | CB | SER | H | 188 | 12.949 | −5.240 | 4.534 | 1.00 | 33.27 | C |
| ATOM | 1456 | OG | SER | H | 188 | 13.611 | −5.711 | 5.702 | 1.00 | 33.64 | O |
| ATOM | 1457 | C | SER | H | 188 | 10.617 | −5.426 | 5.472 | 1.00 | 33.46 | C |
| ATOM | 1458 | O | SER | H | 188 | 9.879 | −6.072 | 4.726 | 1.00 | 33.69 | O |
| ATOM | 1459 | N | LEU | H | 189 | 10.545 | −5.485 | 6.804 | 1.00 | 33.31 | N |
| ATOM | 1460 | CA | LEU | H | 189 | 9.703 | −6.455 | 7.535 | 1.00 | 33.37 | C |
| ATOM | 1461 | CB | LEU | H | 189 | 9.679 | −6.133 | 9.031 | 1.00 | 33.19 | C |
| ATOM | 1462 | CG | LEU | H | 189 | 10.921 | −6.415 | 9.877 | 1.00 | 33.12 | C |
| ATOM | 1463 | CD1 | LEU | H | 189 | 10.676 | −5.969 | 11.307 | 1.00 | 33.26 | C |
| ATOM | 1464 | CD2 | LEU | H | 189 | 11.306 | −7.884 | 9.831 | 1.00 | 32.72 | C |
| ATOM | 1465 | C | LEU | H | 189 | 8.259 | −6.622 | 7.054 | 1.00 | 33.47 | C |
| ATOM | 1466 | O | LEU | H | 189 | 7.688 | −7.710 | 7.173 | 1.00 | 33.68 | O |
| ATOM | 1467 | N | GLY | H | 190 | 7.663 | −5.549 | 6.544 | 1.00 | 33.28 | N |
| ATOM | 1468 | CA | GLY | H | 190 | 6.287 | −5.599 | 6.061 | 1.00 | 32.97 | C |
| ATOM | 1469 | C | GLY | H | 190 | 6.158 | −6.287 | 4.713 | 1.00 | 32.65 | C |
| ATOM | 1470 | O | GLY | H | 190 | 5.084 | −6.795 | 4.371 | 1.00 | 32.66 | O |
| ATOM | 1471 | N | THR | H | 191 | 7.265 | −6.326 | 3.969 | 1.00 | 32.19 | N |
| ATOM | 1472 | CA | THR | H | 191 | 7.259 | −6.648 | 2.543 | 1.00 | 31.82 | C |
| ATOM | 1473 | CB | THR | H | 191 | 7.833 | −5.469 | 1.722 | 1.00 | 31.92 | C |
| ATOM | 1474 | OG1 | THR | H | 191 | 7.468 | −4.230 | 2.346 | 1.00 | 32.88 | O |
| ATOM | 1475 | CG2 | THR | H | 191 | 7.296 | −5.496 | 0.286 | 1.00 | 32.25 | C |
| ATOM | 1476 | C | THR | H | 191 | 8.038 | −7.915 | 2.174 | 1.00 | 31.20 | C |
| ATOM | 1477 | O | THR | H | 191 | 7.552 | −8.742 | 1.398 | 1.00 | 31.20 | O |
| ATOM | 1478 | N | GLN | H | 192 | 9.251 | −8.051 | 2.709 | 1.00 | 30.30 | N |
| ATOM | 1479 | CA | GLN | H | 192 | 10.115 | −9.184 | 2.382 | 1.00 | 29.37 | C |
| ATOM | 1480 | CB | GLN | H | 192 | 11.590 | −8.801 | 2.544 | 1.00 | 29.66 | C |
| ATOM | 1481 | CG | GLN | H | 192 | 12.313 | −8.493 | 1.231 | 1.00 | 31.27 | C |
| ATOM | 1482 | CD | GLN | H | 192 | 12.936 | −9.729 | 0.577 | 1.00 | 32.58 | C |
| ATOM | 1483 | OE1 | GLN | H | 192 | 12.724 | −10.865 | 1.010 | 1.00 | 32.43 | O |
| ATOM | 1484 | NE2 | GLN | H | 192 | 13.716 | −9.503 | −0.474 | 1.00 | 34.61 | N |
| ATOM | 1485 | C | GLN | H | 192 | 9.785 | −10.414 | 3.224 | 1.00 | 28.21 | C |
| ATOM | 1486 | O | GLN | H | 192 | 9.620 | −10.318 | 4.436 | 1.00 | 28.35 | O |
| ATOM | 1487 | N | THR | H | 193 | 9.700 | −11.566 | 2.566 | 1.00 | 26.79 | N |
| ATOM | 1488 | CA | THR | H | 193 | 9.513 | −12.852 | 3.235 | 1.00 | 25.16 | C |
| ATOM | 1489 | CB | THR | H | 193 | 8.880 | −13.875 | 2.274 | 1.00 | 25.28 | C |
| ATOM | 1490 | OG1 | THR | H | 193 | 7.617 | −13.378 | 1.821 | 1.00 | 25.09 | O |
| ATOM | 1491 | CG2 | THR | H | 193 | 8.665 | −15.213 | 2.955 | 1.00 | 25.14 | C |
| ATOM | 1492 | C | THR | H | 193 | 10.834 | −13.408 | 3.766 | 1.00 | 23.87 | C |
| ATOM | 1493 | O | THR | H | 193 | 11.860 | −13.365 | 3.081 | 1.00 | 24.11 | O |
| ATOM | 1494 | N | TYR | H | 194 | 10.808 | −13.927 | 4.990 | 1.00 | 22.11 | N |
| ATOM | 1495 | CA | TYR | H | 194 | 11.958 | −14.634 | 5.544 | 1.00 | 20.42 | C |
| ATOM | 1496 | CB | TYR | H | 194 | 12.599 | −13.847 | 6.684 | 1.00 | 19.71 | C |
| ATOM | 1497 | CG | TYR | H | 194 | 13.101 | −12.491 | 6.257 | 1.00 | 17.97 | C |
| ATOM | 1498 | CD1 | TYR | H | 194 | 12.420 | −11.349 | 6.620 | 1.00 | 16.70 | C |
| ATOM | 1499 | CE1 | TYR | H | 194 | 12.861 | −10.105 | 6.242 | 1.00 | 16.87 | C |
| ATOM | 1500 | CZ | TYR | H | 194 | 14.001 | −9.975 | 5.480 | 1.00 | 16.30 | C |
| ATOM | 1501 | OH | TYR | H | 194 | 14.393 | −8.707 | 5.122 | 1.00 | 17.98 | O |
| ATOM | 1502 | CE2 | TYR | H | 194 | 14.714 | −11.097 | 5.081 | 1.00 | 15.59 | C |
| ATOM | 1503 | CD2 | TYR | H | 194 | 14.262 | −12.354 | 5.476 | 1.00 | 17.50 | C |
| ATOM | 1504 | C | TYR | H | 194 | 11.602 | −16.046 | 5.974 | 1.00 | 20.18 | C |
| ATOM | 1505 | O | TYR | H | 194 | 10.732 | −16.260 | 6.819 | 1.00 | 19.90 | O |
| ATOM | 1506 | N | ILE | H | 195 | 12.270 | −17.007 | 5.354 | 1.00 | 20.09 | N |
| ATOM | 1507 | CA | ILE | H | 195 | 12.056 | −18.410 | 5.636 | 1.00 | 20.45 | C |
| ATOM | 1508 | CB | ILE | H | 195 | 11.398 | −19.146 | 4.436 | 1.00 | 20.62 | C |
| ATOM | 1509 | CG1 | ILE | H | 195 | 10.052 | −18.506 | 4.052 | 1.00 | 20.65 | C |
| ATOM | 1510 | CD1 | ILE | H | 195 | 9.577 | −18.866 | 2.630 | 1.00 | 22.22 | C |
| ATOM | 1511 | CG2 | ILE | H | 195 | 11.202 | −20.631 | 4.735 | 1.00 | 20.63 | C |
| ATOM | 1512 | C | ILE | H | 195 | 13.412 | −19.021 | 5.944 | 1.00 | 20.66 | C |
| ATOM | 1513 | O | ILE | H | 195 | 14.374 | −18.856 | 5.173 | 1.00 | 20.54 | O |
| ATOM | 1514 | N | CYS | H | 196 | 13.505 | −19.705 | 7.081 | 1.00 | 20.83 | N |
| ATOM | 1515 | CA | CYS | H | 196 | 14.689 | −20.486 | 7.373 | 1.00 | 21.80 | C |
| ATOM | 1516 | CB | CYS | H | 196 | 15.053 | −20.462 | 8.866 | 1.00 | 21.52 | C |
| ATOM | 1517 | SG | CYS | H | 196 | 13.899 | −21.360 | 9.889 | 1.00 | 24.39 | S |
| ATOM | 1518 | C | CYS | H | 196 | 14.435 | −21.894 | 6.881 | 1.00 | 21.80 | C |
| ATOM | 1519 | O | CYS | H | 196 | 13.325 | −22.419 | 7.037 | 1.00 | 21.73 | O |
| ATOM | 1520 | N | ASN | H | 197 | 15.454 | −22.483 | 6.256 | 1.00 | 22.18 | N |
| ATOM | 1521 | CA | ASN | H | 197 | 15.355 | −23.820 | 5.680 | 1.00 | 22.60 | C |
| ATOM | 1522 | CB | ASN | H | 197 | 15.760 | −23.821 | 4.196 | 1.00 | 22.72 | C |
| ATOM | 1523 | CG | ASN | H | 197 | 15.267 | −22.586 | 3.436 | 1.00 | 22.83 | C |
| ATOM | 1524 | OD1 | ASN | H | 197 | 16.046 | −21.680 | 3.147 | 1.00 | 24.31 | O |
| ATOM | 1525 | ND2 | ASN | H | 197 | 13.981 | −22.551 | 3.109 | 1.00 | 20.96 | N |
| ATOM | 1526 | C | ASN | H | 197 | 16.257 | −24.768 | 6.466 | 1.00 | 23.04 | C |
| ATOM | 1527 | O | ASN | H | 197 | 17.478 | −24.611 | 6.463 | 1.00 | 22.69 | O |
| ATOM | 1528 | N | VAL | H | 198 | 15.658 | −25.754 | 7.126 | 1.00 | 23.11 | N |
| ATOM | 1529 | CA | VAL | H | 198 | 16.405 | −26.634 | 8.024 | 1.00 | 23.99 | C |
| ATOM | 1530 | CB | VAL | H | 198 | 15.764 | −26.667 | 9.443 | 1.00 | 23.85 | C |
| ATOM | 1531 | CG1 | VAL | H | 198 | 16.608 | −27.493 | 10.402 | 1.00 | 24.25 | C |
| ATOM | 1532 | CG2 | VAL | H | 198 | 15.590 | −25.254 | 9.977 | 1.00 | 23.46 | C |
| ATOM | 1533 | C | VAL | H | 198 | 16.484 | −28.043 | 7.428 | 1.00 | 24.70 | C |
| ATOM | 1534 | O | VAL | H | 198 | 15.458 | −28.646 | 7.116 | 1.00 | 24.58 | O |
| ATOM | 1535 | N | ASN | H | 199 | 17.701 | −28.547 | 7.236 | 1.00 | 25.67 | N |
| ATOM | 1536 | CA | ASN | H | 199 | 17.886 | −29.927 | 6.792 | 1.00 | 26.81 | C |
| ATOM | 1537 | CB | ASN | H | 199 | 18.632 | −30.007 | 5.442 | 1.00 | 27.49 | C |
| ATOM | 1538 | CG | ASN | H | 199 | 18.541 | −31.398 | 4.786 | 1.00 | 29.76 | C |
| ATOM | 1539 | OD1 | ASN | H | 199 | 17.862 | −32.308 | 5.281 | 1.00 | 32.55 | O |
| ATOM | 1540 | ND2 | ASN | H | 199 | 19.224 | −31.558 | 3.658 | 1.00 | 31.95 | N |
| ATOM | 1541 | C | ASN | H | 199 | 18.608 | −30.736 | 7.856 | 1.00 | 26.75 | C |
| ATOM | 1542 | O | ASN | H | 199 | 19.659 | −30.336 | 8.338 | 1.00 | 27.08 | O |
| ATOM | 1543 | N | HIS | H | 200 | 18.006 | −31.857 | 8.238 | 1.00 | 27.01 | N |
| ATOM | 1544 | CA | HIS | H | 200 | 18.633 | −32.819 | 9.128 | 1.00 | 27.15 | C |
| ATOM | 1545 | CB | HIS | H | 200 | 17.815 | −32.982 | 10.411 | 1.00 | 26.45 | C |
| ATOM | 1546 | CG | HIS | H | 200 | 18.482 | −33.830 | 11.452 | 1.00 | 24.91 | C |
| ATOM | 1547 | ND1 | HIS | H | 200 | 17.884 | −34.947 | 11.995 | 1.00 | 23.35 | N |
| ATOM | 1548 | CE1 | HIS | H | 200 | 18.693 | −35.487 | 12.890 | 1.00 | 23.96 | C |
| ATOM | 1549 | NE2 | HIS | H | 200 | 19.798 | −34.762 | 12.945 | 1.00 | 22.63 | N |
| ATOM | 1550 | CD2 | HIS | H | 200 | 19.689 | −33.718 | 12.058 | 1.00 | 23.70 | C |
| ATOM | 1551 | C | HIS | H | 200 | 18.727 | −34.142 | 8.390 | 1.00 | 27.80 | C |
| ATOM | 1552 | O | HIS | H | 200 | 17.794 | −34.946 | 8.427 | 1.00 | 27.49 | O |
| ATOM | 1553 | N | LYS | H | 201 | 19.856 | −34.355 | 7.716 | 1.00 | 28.82 | N |
| ATOM | 1554 | CA | LYS | H | 201 | 20.091 | −35.576 | 6.928 | 1.00 | 30.01 | C |
| ATOM | 1555 | CB | LYS | H | 201 | 21.491 | −35.534 | 6.293 | 1.00 | 30.20 | C |
| ATOM | 1556 | CG | LYS | H | 201 | 21.956 | −36.853 | 5.664 | 1.00 | 32.73 | C |
| ATOM | 1557 | CD | LYS | H | 201 | 21.141 | −37.211 | 4.413 | 1.00 | 34.68 | C |
| ATOM | 1558 | CE | LYS | H | 201 | 21.412 | −38.642 | 3.963 | 1.00 | 35.79 | C |
| ATOM | 1559 | NZ | LYS | H | 201 | 20.609 | −38.982 | 2.750 | 1.00 | 36.64 | N |
| ATOM | 1560 | C | LYS | H | 201 | 19.870 | −36.902 | 7.694 | 1.00 | 30.13 | C |
| ATOM | 1561 | O | LYS | H | 201 | 19.187 | −37.794 | 7.183 | 1.00 | 30.23 | O |
| ATOM | 1562 | N | PRO | H | 202 | 20.439 | −37.040 | 8.919 | 1.00 | 30.41 | N |
| ATOM | 1563 | CA | PRO | H | 202 | 20.303 | −38.333 | 9.601 | 1.00 | 30.47 | C |
| ATOM | 1564 | CB | PRO | H | 202 | 21.027 | −38.106 | 10.933 | 1.00 | 30.40 | C |
| ATOM | 1565 | CG | PRO | H | 202 | 22.043 | −37.073 | 10.629 | 1.00 | 30.30 | C |
| ATOM | 1566 | CD | PRO | H | 202 | 21.353 | −36.143 | 9.654 | 1.00 | 30.34 | C |
| ATOM | 1567 | C | PRO | H | 202 | 18.862 | −38.804 | 9.832 | 1.00 | 30.65 | C |
| ATOM | 1568 | O | PRO | H | 202 | 18.642 | −39.993 | 10.039 | 1.00 | 30.70 | O |
| ATOM | 1569 | N | SER | H | 203 | 17.895 | −37.893 | 9.778 | 1.00 | 31.00 | N |
| ATOM | 1570 | CA | SER | H | 203 | 16.489 | −38.259 | 9.947 | 1.00 | 31.47 | C |
| ATOM | 1571 | CB | SER | H | 203 | 15.884 | −37.476 | 11.115 | 1.00 | 31.45 | C |
| ATOM | 1572 | OG | SER | H | 203 | 15.804 | −36.097 | 10.800 | 1.00 | 30.77 | O |
| ATOM | 1573 | C | SER | H | 203 | 15.629 | −38.051 | 8.689 | 1.00 | 31.96 | C |
| ATOM | 1574 | O | SER | H | 203 | 14.399 | −38.178 | 8.748 | 1.00 | 32.10 | O |
| ATOM | 1575 | N | ASN | H | 204 | 16.271 | −37.721 | 7.569 | 1.00 | 32.50 | N |
| ATOM | 1576 | CA | ASN | H | 204 | 15.570 | −37.288 | 6.345 | 1.00 | 32.83 | C |
| ATOM | 1577 | CB | ASN | H | 204 | 14.947 | −38.477 | 5.597 | 1.00 | 33.16 | C |
| ATOM | 1578 | CG | ASN | H | 204 | 15.948 | −39.593 | 5.327 | 1.00 | 34.20 | C |
| ATOM | 1579 | OD1 | ASN | H | 204 | 17.042 | −39.358 | 4.801 | 1.00 | 36.20 | O |
| ATOM | 1580 | ND2 | ASN | H | 204 | 15.573 | −40.820 | 5.679 | 1.00 | 35.53 | N |
| ATOM | 1581 | C | ASN | H | 204 | 14.528 | −36.200 | 6.623 | 1.00 | 32.76 | C |
| ATOM | 1582 | O | ASN | H | 204 | 13.388 | −36.272 | 6.163 | 1.00 | 32.58 | O |
| ATOM | 1583 | N | THR | H | 205 | 14.937 | −35.192 | 7.386 | 1.00 | 32.72 | N |
| ATOM | 1584 | CA | THR | H | 205 | 14.049 | −34.097 | 7.770 | 1.00 | 32.77 | C |
| ATOM | 1585 | CB | THR | H | 205 | 14.099 | −33.841 | 9.302 | 1.00 | 32.91 | C |
| ATOM | 1586 | OG1 | THR | H | 205 | 13.480 | −34.939 | 9.986 | 1.00 | 33.71 | O |
| ATOM | 1587 | CG2 | THR | H | 205 | 13.364 | −32.551 | 9.681 | 1.00 | 33.26 | C |
| ATOM | 1588 | C | THR | H | 205 | 14.388 | −32.821 | 7.004 | 1.00 | 32.34 | C |
| ATOM | 1589 | O | THR | H | 205 | 15.533 | −32.369 | 7.006 | 1.00 | 32.53 | O |
| ATOM | 1590 | N | LYS | H | 206 | 13.386 | −32.264 | 6.334 | 1.00 | 31.76 | N |
| ATOM | 1591 | CA | LYS | H | 206 | 13.506 | −30.969 | 5.670 | 1.00 | 31.43 | C |
| ATOM | 1592 | CB | LYS | H | 206 | 13.545 | −31.104 | 4.139 | 1.00 | 31.46 | C |
| ATOM | 1593 | CG | LYS | H | 206 | 14.729 | −31.932 | 3.622 | 1.00 | 33.19 | C |
| ATOM | 1594 | CD | LYS | H | 206 | 14.966 | −31.765 | 2.114 | 1.00 | 36.15 | C |
| ATOM | 1595 | CE | LYS | H | 206 | 15.859 | −30.563 | 1.806 | 1.00 | 36.72 | C |
| ATOM | 1596 | NZ | LYS | H | 206 | 16.341 | −30.565 | 0.387 | 1.00 | 38.11 | N |
| ATOM | 1597 | C | LYS | H | 206 | 12.336 | −30.106 | 6.118 | 1.00 | 30.67 | C |
| ATOM | 1598 | O | LYS | H | 206 | 11.178 | −30.494 | 5.976 | 1.00 | 30.46 | O |
| ATOM | 1599 | N | VAL | H | 207 | 12.657 | −28.951 | 6.693 | 1.00 | 29.80 | N |
| ATOM | 1600 | CA | VAL | H | 207 | 11.658 | −28.044 | 7.242 | 1.00 | 29.26 | C |
| ATOM | 1601 | CB | VAL | H | 207 | 11.649 | −28.064 | 8.801 | 1.00 | 29.48 | C |
| ATOM | 1602 | CG1 | VAL | H | 207 | 10.660 | −27.037 | 9.374 | 1.00 | 29.44 | C |
| ATOM | 1603 | CG2 | VAL | H | 207 | 11.318 | −29.462 | 9.328 | 1.00 | 29.89 | C |
| ATOM | 1604 | C | VAL | H | 207 | 11.949 | −26.643 | 6.743 | 1.00 | 28.81 | C |
| ATOM | 1605 | O | VAL | H | 207 | 13.086 | −26.176 | 6.801 | 1.00 | 28.82 | O |
| ATOM | 1606 | N | ASP | H | 208 | 10.919 | −25.990 | 6.225 | 1.00 | 28.36 | N |
| ATOM | 1607 | CA | ASP | H | 208 | 10.970 | −24.569 | 5.921 | 1.00 | 28.26 | C |
| ATOM | 1608 | CB | ASP | H | 208 | 10.537 | −24.306 | 4.472 | 1.00 | 28.22 | C |
| ATOM | 1609 | CG | ASP | H | 208 | 11.468 | −24.956 | 3.445 | 1.00 | 29.38 | C |
| ATOM | 1610 | OD1 | ASP | H | 208 | 12.653 | −25.205 | 3.759 | 1.00 | 29.41 | O |
| ATOM | 1611 | OD2 | ASP | H | 208 | 11.010 | −25.214 | 2.306 | 1.00 | 30.60 | O |
| ATOM | 1612 | C | ASP | H | 208 | 10.034 | −23.858 | 6.890 | 1.00 | 27.99 | C |
| ATOM | 1613 | O | ASP | H | 208 | 8.854 | −24.199 | 6.972 | 1.00 | 28.47 | O |
| ATOM | 1614 | N | LYS | H | 209 | 10.549 | −22.883 | 7.631 | 1.00 | 27.50 | N |
| ATOM | 1615 | CA | LYS | H | 209 | 9.720 | −22.137 | 8.580 | 1.00 | 27.11 | C |
| ATOM | 1616 | CB | LYS | H | 209 | 10.223 | −22.350 | 10.012 | 1.00 | 26.99 | C |
| ATOM | 1617 | CG | LYS | H | 209 | 9.213 | −22.046 | 11.095 | 1.00 | 27.19 | C |
| ATOM | 1618 | CD | LYS | H | 209 | 8.162 | −23.142 | 11.195 | 1.00 | 27.14 | C |
| ATOM | 1619 | CE | LYS | H | 209 | 6.978 | −22.672 | 12.014 | 1.00 | 28.08 | C |
| ATOM | 1620 | NZ | LYS | H | 209 | 5.909 | −23.706 | 12.086 | 1.00 | 26.89 | N |
| ATOM | 1621 | C | LYS | H | 209 | 9.702 | −20.653 | 8.234 | 1.00 | 26.98 | C |
| ATOM | 1622 | O | LYS | H | 209 | 10.735 | −19.980 | 8.302 | 1.00 | 26.38 | O |
| ATOM | 1623 | N | ARG | H | 210 | 8.532 | −20.150 | 7.850 | 1.00 | 26.94 | N |
| ATOM | 1624 | CA | ARG | H | 210 | 8.368 | −18.728 | 7.567 | 1.00 | 27.82 | C |
| ATOM | 1625 | CB | ARG | H | 210 | 7.072 | −18.468 | 6.792 | 1.00 | 28.19 | C |
| ATOM | 1626 | CG | ARG | H | 210 | 6.971 | −17.042 | 6.277 | 1.00 | 29.43 | C |
| ATOM | 1627 | CD | ARG | H | 210 | 5.673 | −16.767 | 5.547 | 1.00 | 32.71 | C |
| ATOM | 1628 | NE | ARG | H | 210 | 5.847 | −15.636 | 4.640 | 1.00 | 35.08 | N |
| ATOM | 1629 | CZ | ARG | H | 210 | 4.928 | −14.710 | 4.378 | 1.00 | 36.95 | C |
| ATOM | 1630 | NH1 | ARG | H | 210 | 3.730 | −14.748 | 4.958 | 1.00 | 37.53 | N |
| ATOM | 1631 | NH2 | ARG | H | 210 | 5.217 | −13.730 | 3.532 | 1.00 | 37.65 | N |
| ATOM | 1632 | C | ARG | H | 210 | 8.376 | −17.910 | 8.862 | 1.00 | 28.22 | C |
| ATOM | 1633 | O | ARG | H | 210 | 7.590 | −18.174 | 9.772 | 1.00 | 28.13 | O |
| ATOM | 1634 | N | VAL | H | 211 | 9.263 | −16.920 | 8.935 | 1.00 | 28.65 | N |
| ATOM | 1635 | CA | VAL | H | 211 | 9.381 | −16.074 | 10.125 | 1.00 | 29.41 | C |
| ATOM | 1636 | CB | VAL | H | 211 | 10.851 | −15.962 | 10.619 | 1.00 | 29.28 | C |
| ATOM | 1637 | CG1 | VAL | H | 211 | 10.968 | −15.032 | 11.821 | 1.00 | 29.13 | C |
| ATOM | 1638 | CG2 | VAL | H | 211 | 11.378 | −17.333 | 10.971 | 1.00 | 28.54 | C |
| ATOM | 1639 | C | VAL | H | 211 | 8.774 | −14.701 | 9.849 | 1.00 | 30.44 | C |
| ATOM | 1640 | O | VAL | H | 211 | 9.305 | −13.917 | 9.054 | 1.00 | 30.01 | O |
| ATOM | 1641 | N | GLU | H | 212 | 7.648 | −14.432 | 10.505 | 1.00 | 31.44 | N |
| ATOM | 1642 | CA | GLU | H | 212 | 6.936 | −13.173 | 10.319 | 1.00 | 32.99 | C |
| ATOM | 1643 | CB | GLU | H | 212 | 5.683 | −13.375 | 9.454 | 1.00 | 33.21 | C |
| ATOM | 1644 | CG | GLU | H | 212 | 4.752 | −14.511 | 9.887 | 1.00 | 33.90 | C |
| ATOM | 1645 | CD | GLU | H | 212 | 3.579 | −14.686 | 8.938 | 1.00 | 34.12 | C |
| ATOM | 1646 | OE1 | GLU | H | 212 | 2.439 | −14.799 | 9.429 | 1.00 | 35.34 | O |
| ATOM | 1647 | OE2 | GLU | H | 212 | 3.796 | −14.697 | 7.706 | 1.00 | 33.69 | O |
| ATOM | 1648 | C | GLU | H | 212 | 6.602 | −12.499 | 11.647 | 1.00 | 33.78 | C |
| ATOM | 1649 | O | GLU | H | 212 | 6.395 | −13.183 | 12.654 | 1.00 | 33.99 | O |
| ATOM | 1650 | N | PRO | H | 213 | 6.565 | −11.152 | 11.655 | 1.00 | 34.60 | N |
| ATOM | 1651 | CA | PRO | H | 213 | 6.283 | −10.408 | 12.882 | 1.00 | 35.51 | C |
| ATOM | 1652 | CB | PRO | H | 213 | 6.198 | −8.948 | 12.403 | 1.00 | 35.45 | C |
| ATOM | 1653 | CG | PRO | H | 213 | 6.057 | −9.025 | 10.909 | 1.00 | 35.20 | C |
| ATOM | 1654 | CD | PRO | H | 213 | 6.821 | −10.244 | 10.522 | 1.00 | 34.69 | C |
| ATOM | 1655 | C | PRO | H | 213 | 4.970 | −10.825 | 13.545 | 1.00 | 36.19 | C |
| ATOM | 1656 | O | PRO | H | 213 | 4.056 | −11.295 | 12.860 | 1.00 | 36.09 | O |
| ATOM | 1657 | N | LYS | H | 214 | 4.914 | −10.667 | 14.870 | 1.00 | 37.08 | N |
| ATOM | 1658 | CA | LYS | H | 214 | 3.698 | −10.871 | 15.692 | 1.00 | 37.92 | C |
| ATOM | 1659 | CB | LYS | H | 214 | 2.492 | −10.067 | 15.158 | 1.00 | 37.85 | C |
| ATOM | 1660 | CG | LYS | H | 214 | 2.429 | −8.614 | 15.648 | 1.00 | 39.35 | C |
| ATOM | 1661 | CD | LYS | H | 214 | 3.763 | −7.873 | 15.459 | 1.00 | 40.84 | C |
| ATOM | 1662 | CE | LYS | H | 214 | 4.167 | −7.144 | 16.739 | 1.00 | 42.01 | C |
| ATOM | 1663 | NZ | LYS | H | 214 | 5.647 | −7.032 | 16.892 | 1.00 | 41.84 | N |
| ATOM | 1664 | C | LYS | H | 214 | 3.343 | −12.339 | 15.897 | 1.00 | 38.07 | C |
| ATOM | 1665 | O | LYS | H | 214 | 3.715 | −12.931 | 16.913 | 1.00 | 38.40 | O |
| ATOM | 1666 | N | ASP | L | 1 | 54.126 | −38.930 | 31.306 | 1.00 | 32.08 | N |
| ATOM | 1667 | CA | ASP | L | 1 | 54.170 | −38.386 | 29.916 | 1.00 | 31.79 | C |
| ATOM | 1668 | CB | ASP | L | 1 | 52.767 | −38.442 | 29.281 | 1.00 | 32.39 | C |
| ATOM | 1669 | CG | ASP | L | 1 | 52.103 | −39.836 | 29.408 | 1.00 | 34.52 | C |
| ATOM | 1670 | OD1 | ASP | L | 1 | 52.812 | −40.841 | 29.696 | 1.00 | 35.92 | O |
| ATOM | 1671 | OD2 | ASP | L | 1 | 50.863 | −39.936 | 29.208 | 1.00 | 36.28 | O |
| ATOM | 1672 | C | ASP | L | 1 | 54.736 | −36.955 | 29.943 | 1.00 | 31.06 | C |
| ATOM | 1673 | O | ASP | L | 1 | 54.734 | −36.307 | 30.997 | 1.00 | 31.34 | O |
| ATOM | 1674 | N | VAL | L | 2 | 55.250 | −36.476 | 28.806 | 1.00 | 29.56 | N |
| ATOM | 1675 | CA | VAL | L | 2 | 55.848 | −35.148 | 28.748 | 1.00 | 27.92 | C |
| ATOM | 1676 | CB | VAL | L | 2 | 56.853 | −35.007 | 27.558 | 1.00 | 28.22 | C |
| ATOM | 1677 | CG1 | VAL | L | 2 | 57.470 | −33.608 | 27.527 | 1.00 | 27.28 | C |
| ATOM | 1678 | CG2 | VAL | L | 2 | 57.932 | −36.078 | 27.613 | 1.00 | 27.73 | C |
| ATOM | 1679 | C | VAL | L | 2 | 54.725 | −34.115 | 28.605 | 1.00 | 27.26 | C |
| ATOM | 1680 | O | VAL | L | 2 | 54.141 | −33.968 | 27.523 | 1.00 | 26.52 | O |
| ATOM | 1681 | N | VAL | L | 3 | 54.401 | −33.414 | 29.689 | 1.00 | 25.73 | N |
| ATOM | 1682 | CA | VAL | L | 3 | 53.328 | −32.425 | 29.609 | 1.00 | 25.36 | C |
| ATOM | 1683 | CB | VAL | L | 3 | 52.722 | −32.063 | 30.985 | 1.00 | 26.00 | C |
| ATOM | 1684 | CG1 | VAL | L | 3 | 51.495 | −31.174 | 30.790 | 1.00 | 26.66 | C |
| ATOM | 1685 | CG2 | VAL | L | 3 | 52.337 | −33.323 | 31.761 | 1.00 | 26.36 | C |
| ATOM | 1686 | C | VAL | L | 3 | 53.850 | −31.169 | 28.913 | 1.00 | 24.22 | C |
| ATOM | 1687 | O | VAL | L | 3 | 54.939 | −30.701 | 29.205 | 1.00 | 23.47 | O |
| ATOM | 1688 | N | MET | L | 4 | 53.070 | −30.660 | 27.976 | 1.00 | 23.54 | N |
| ATOM | 1689 | CA | MET | L | 4 | 53.404 | −29.449 | 27.244 | 1.00 | 23.66 | C |
| ATOM | 1690 | CB | MET | L | 4 | 53.237 | −29.717 | 25.743 | 1.00 | 24.04 | C |
| ATOM | 1691 | CG | MET | L | 4 | 53.938 | −30.989 | 25.271 | 1.00 | 25.28 | C |
| ATOM | 1692 | SD | MET | L | 4 | 55.680 | −30.619 | 24.991 | 1.00 | 27.49 | S |
| ATOM | 1693 | CE | MET | L | 4 | 55.551 | −29.935 | 23.372 | 1.00 | 28.10 | C |
| ATOM | 1694 | C | MET | L | 4 | 52.455 | −28.329 | 27.675 | 1.00 | 23.22 | C |
| ATOM | 1695 | O | MET | L | 4 | 51.257 | −28.492 | 27.586 | 1.00 | 22.33 | O |
| ATOM | 1696 | N | THR | L | 5 | 53.003 | −27.200 | 28.129 | 1.00 | 23.05 | N |
| ATOM | 1697 | CA | THR | L | 5 | 52.210 | −26.070 | 28.638 | 1.00 | 23.13 | C |
| ATOM | 1698 | CB | THR | L | 5 | 52.634 | −25.721 | 30.099 | 1.00 | 23.65 | C |
| ATOM | 1699 | OG1 | THR | L | 5 | 52.500 | −26.878 | 30.926 | 1.00 | 23.42 | O |
| ATOM | 1700 | CG2 | THR | L | 5 | 51.788 | −24.597 | 30.701 | 1.00 | 23.80 | C |
| ATOM | 1701 | C | THR | L | 5 | 52.386 | −24.835 | 27.743 | 1.00 | 22.89 | C |
| ATOM | 1702 | O | THR | L | 5 | 53.502 | −24.313 | 27.612 | 1.00 | 22.15 | O |
| ATOM | 1703 | N | GLN | L | 6 | 51.291 | −24.377 | 27.140 | 1.00 | 22.64 | N |
| ATOM | 1704 | CA | GLN | L | 6 | 51.320 | −23.200 | 26.272 | 1.00 | 23.06 | C |
| ATOM | 1705 | CB | GLN | L | 6 | 50.523 | −23.463 | 24.998 | 1.00 | 22.94 | C |
| ATOM | 1706 | CG | GLN | L | 6 | 51.260 | −24.327 | 23.989 | 1.00 | 22.13 | C |
| ATOM | 1707 | CD | GLN | L | 6 | 50.448 | −24.537 | 22.715 | 1.00 | 22.43 | C |
| ATOM | 1708 | OE1 | GLN | L | 6 | 49.884 | −25.613 | 22.494 | 1.00 | 20.27 | O |
| ATOM | 1709 | NE2 | GLN | L | 6 | 50.392 | −23.516 | 21.875 | 1.00 | 20.32 | N |
| ATOM | 1710 | C | GLN | L | 6 | 50.781 | −21.945 | 26.950 | 1.00 | 23.63 | C |
| ATOM | 1711 | O | GLN | L | 6 | 49.782 | −22.008 | 27.664 | 1.00 | 22.72 | O |
| ATOM | 1712 | N | THR | L | 7 | 51.443 | −20.811 | 26.716 | 1.00 | 24.12 | N |
| ATOM | 1713 | CA | THR | L | 7 | 50.930 | −19.511 | 27.148 | 1.00 | 25.31 | C |
| ATOM | 1714 | CB | THR | L | 7 | 51.567 | −19.007 | 28.470 | 1.00 | 25.62 | C |
| ATOM | 1715 | OG1 | THR | L | 7 | 52.986 | −18.984 | 28.321 | 1.00 | 28.07 | O |
| ATOM | 1716 | CG2 | THR | L | 7 | 51.184 | −19.908 | 29.676 | 1.00 | 26.90 | C |
| ATOM | 1717 | C | THR | L | 7 | 51.163 | −18.484 | 26.032 | 1.00 | 25.38 | C |
| ATOM | 1718 | O | THR | L | 7 | 52.094 | −18.646 | 25.221 | 1.00 | 24.86 | O |
| ATOM | 1719 | N | PRO | L | 8 | 50.280 | −17.470 | 25.940 | 1.00 | 24.93 | N |
| ATOM | 1720 | CA | PRO | L | 8 | 49.040 | −17.329 | 26.715 | 1.00 | 24.79 | C |
| ATOM | 1721 | CB | PRO | L | 8 | 48.649 | −15.874 | 26.462 | 1.00 | 24.75 | C |
| ATOM | 1722 | CG | PRO | L | 8 | 49.113 | −15.640 | 25.043 | 1.00 | 24.79 | C |
| ATOM | 1723 | CD | PRO | L | 8 | 50.426 | −16.393 | 24.937 | 1.00 | 25.00 | C |
| ATOM | 1724 | C | PRO | L | 8 | 47.969 | −18.255 | 26.142 | 1.00 | 25.21 | C |
| ATOM | 1725 | O | PRO | L | 8 | 48.187 | −18.855 | 25.096 | 1.00 | 24.69 | O |
| ATOM | 1726 | N | LEU | L | 9 | 46.813 | −18.351 | 26.796 | 1.00 | 25.38 | N |
| ATOM | 1727 | CA | LEU | L | 9 | 45.714 | −19.158 | 26.242 | 1.00 | 26.07 | C |
| ATOM | 1728 | CB | LEU | L | 9 | 44.666 | −19.480 | 27.320 | 1.00 | 26.98 | C |
| ATOM | 1729 | CG | LEU | L | 9 | 45.178 | −20.057 | 28.649 | 1.00 | 29.28 | C |
| ATOM | 1730 | CD1 | LEU | L | 9 | 44.038 | −20.217 | 29.654 | 1.00 | 33.47 | C |
| ATOM | 1731 | CD2 | LEU | L | 9 | 45.926 | −21.400 | 28.419 | 1.00 | 32.45 | C |
| ATOM | 1732 | C | LEU | L | 9 | 45.067 | −18.456 | 25.058 | 1.00 | 25.17 | C |
| ATOM | 1733 | O | LEU | L | 9 | 44.629 | −19.105 | 24.102 | 1.00 | 25.13 | O |
| ATOM | 1734 | N | SER | L | 10 | 45.021 | −17.126 | 25.123 | 1.00 | 24.64 | N |
| ATOM | 1735 | CA | SER | L | 10 | 44.427 | −16.289 | 24.074 | 1.00 | 24.30 | C |
| ATOM | 1736 | CB | SER | L | 10 | 43.079 | −15.740 | 24.568 | 1.00 | 24.59 | C |
| ATOM | 1737 | OG | SER | L | 10 | 42.640 | −14.688 | 23.738 | 1.00 | 25.37 | O |
| ATOM | 1738 | C | SER | L | 10 | 45.358 | −15.120 | 23.727 | 1.00 | 23.68 | C |
| ATOM | 1739 | O | SER | L | 10 | 45.931 | −14.497 | 24.633 | 1.00 | 24.34 | O |
| ATOM | 1740 | N | LEU | L | 11 | 45.511 | −14.818 | 22.436 | 1.00 | 22.47 | N |
| ATOM | 1741 | CA | LEU | L | 11 | 46.318 | −13.658 | 22.010 | 1.00 | 21.90 | C |
| ATOM | 1742 | CB | LEU | L | 11 | 47.757 | −14.044 | 21.582 | 1.00 | 22.58 | C |
| ATOM | 1743 | CG | LEU | L | 11 | 48.762 | −12.889 | 21.299 | 1.00 | 24.27 | C |
| ATOM | 1744 | CD1 | LEU | L | 11 | 49.129 | −12.069 | 22.538 | 1.00 | 25.77 | C |
| ATOM | 1745 | CD2 | LEU | L | 11 | 50.049 | −13.399 | 20.619 | 1.00 | 27.03 | C |
| ATOM | 1746 | C | LEU | L | 11 | 45.616 | −12.855 | 20.916 | 1.00 | 21.33 | C |
| ATOM | 1747 | O | LEU | L | 11 | 45.687 | −13.208 | 19.715 | 1.00 | 20.20 | O |
| ATOM | 1748 | N | PRO | L | 12 | 44.891 | −11.798 | 21.331 | 1.00 | 20.46 | N |
| ATOM | 1749 | CA | PRO | L | 12 | 44.413 | −10.801 | 20.383 | 1.00 | 20.25 | C |
| ATOM | 1750 | CB | PRO | L | 12 | 43.585 | −9.841 | 21.252 | 1.00 | 20.42 | C |
| ATOM | 1751 | CG | PRO | L | 12 | 43.233 | −10.617 | 22.490 | 1.00 | 20.50 | C |
| ATOM | 1752 | CD | PRO | L | 12 | 44.384 | −11.568 | 22.700 | 1.00 | 20.53 | C |
| ATOM | 1753 | C | PRO | L | 12 | 45.590 | −10.059 | 19.755 | 1.00 | 20.01 | C |
| ATOM | 1754 | O | PRO | L | 12 | 46.499 | −9.602 | 20.456 | 1.00 | 20.07 | O |
| ATOM | 1755 | N | VAL | L | 13 | 45.572 | −9.957 | 18.435 | 1.00 | 19.71 | N |
| ATOM | 1756 | CA | VAL | L | 13 | 46.663 | −9.341 | 17.687 | 1.00 | 19.38 | C |
| ATOM | 1757 | CB | VAL | L | 13 | 47.511 | −10.391 | 16.920 | 1.00 | 19.20 | C |
| ATOM | 1758 | CG1 | VAL | L | 13 | 48.236 | −11.320 | 17.877 | 1.00 | 19.24 | C |
| ATOM | 1759 | CG2 | VAL | L | 13 | 46.639 | −11.198 | 15.958 | 1.00 | 19.92 | C |
| ATOM | 1760 | C | VAL | L | 13 | 46.102 | −8.346 | 16.669 | 1.00 | 19.43 | C |
| ATOM | 1761 | O | VAL | L | 13 | 45.050 | −8.575 | 16.081 | 1.00 | 18.91 | O |
| ATOM | 1762 | N | THR | L | 14 | 46.832 | −7.251 | 16.472 | 1.00 | 19.46 | N |
| ATOM | 1763 | CA | THR | L | 14 | 46.520 | −6.239 | 15.467 | 1.00 | 19.75 | C |
| ATOM | 1764 | CB | THR | L | 14 | 46.775 | −4.825 | 16.082 | 1.00 | 19.93 | C |
| ATOM | 1765 | OG1 | THR | L | 14 | 46.144 | −4.746 | 17.367 | 1.00 | 20.61 | O |
| ATOM | 1766 | CG2 | THR | L | 14 | 46.254 | −3.707 | 15.200 | 1.00 | 20.38 | C |
| ATOM | 1767 | C | THR | L | 14 | 47.441 | −6.490 | 14.261 | 1.00 | 19.43 | C |
| ATOM | 1768 | O | THR | L | 14 | 48.658 | −6.531 | 14.415 | 1.00 | 19.76 | O |
| ATOM | 1769 | N | PRO | L | 15 | 46.872 | −6.664 | 13.051 | 1.00 | 19.16 | N |
| ATOM | 1770 | CA | PRO | L | 15 | 47.702 | −6.896 | 11.866 | 1.00 | 18.88 | C |
| ATOM | 1771 | CB | PRO | L | 15 | 46.690 | −6.805 | 10.717 | 1.00 | 18.96 | C |
| ATOM | 1772 | CG | PRO | L | 15 | 45.427 | −7.361 | 11.353 | 1.00 | 19.68 | C |
| ATOM | 1773 | CD | PRO | L | 15 | 45.432 | −6.714 | 12.720 | 1.00 | 19.68 | C |
| ATOM | 1774 | C | PRO | L | 15 | 48.837 | −5.878 | 11.674 | 1.00 | 18.26 | C |
| ATOM | 1775 | O | PRO | L | 15 | 48.607 | −4.658 | 11.738 | 1.00 | 18.14 | O |
| ATOM | 1776 | N | GLY | L | 16 | 50.040 | −6.393 | 11.436 | 1.00 | 17.18 | N |
| ATOM | 1777 | CA | GLY | L | 16 | 51.237 | −5.578 | 11.281 | 1.00 | 16.59 | C |
| ATOM | 1778 | C | GLY | L | 16 | 52.177 | −5.632 | 12.481 | 1.00 | 16.04 | C |
| ATOM | 1779 | O | GLY | L | 16 | 53.371 | −5.421 | 12.338 | 1.00 | 15.68 | O |
| ATOM | 1780 | N | GLU | L | 17 | 51.643 | −5.922 | 13.663 | 1.00 | 15.63 | N |
| ATOM | 1781 | CA | GLU | L | 17 | 52.468 | −5.985 | 14.865 | 1.00 | 16.45 | C |
| ATOM | 1782 | CB | GLU | L | 17 | 51.628 | −5.631 | 16.093 | 1.00 | 16.72 | C |
| ATOM | 1783 | CG | GLU | L | 17 | 50.742 | −6.757 | 16.605 | 1.00 | 18.29 | C |
| ATOM | 1784 | CD | GLU | L | 17 | 50.032 | −6.399 | 17.910 | 1.00 | 22.13 | C |
| ATOM | 1785 | OE1 | GLU | L | 17 | 48.946 | −6.951 | 18.168 | 1.00 | 21.59 | O |
| ATOM | 1786 | OE2 | GLU | L | 17 | 50.563 | −5.566 | 18.677 | 1.00 | 23.32 | O |
| ATOM | 1787 | C | GLU | L | 17 | 53.082 | −7.394 | 15.030 | 1.00 | 16.89 | C |
| ATOM | 1788 | O | GLU | L | 17 | 52.517 | −8.375 | 14.511 | 1.00 | 16.35 | O |
| ATOM | 1789 | N | PRO | L | 18 | 54.211 | −7.497 | 15.764 | 1.00 | 17.04 | N |
| ATOM | 1790 | CA | PRO | L | 18 | 54.735 | −8.832 | 16.085 | 1.00 | 17.61 | C |
| ATOM | 1791 | CB | PRO | L | 18 | 56.140 | −8.565 | 16.641 | 1.00 | 17.80 | C |
| ATOM | 1792 | CG | PRO | L | 18 | 56.313 | −7.069 | 16.694 | 1.00 | 17.67 | C |
| ATOM | 1793 | CD | PRO | L | 18 | 55.009 | −6.411 | 16.366 | 1.00 | 17.21 | C |
| ATOM | 1794 | C | PRO | L | 18 | 53.904 | −9.579 | 17.121 | 1.00 | 18.60 | C |
| ATOM | 1795 | O | PRO | L | 18 | 53.145 | −8.962 | 17.882 | 1.00 | 18.47 | O |
| ATOM | 1796 | N | ALA | L | 19 | 54.045 | −10.905 | 17.122 | 1.00 | 18.92 | N |
| ATOM | 1797 | CA | ALA | L | 19 | 53.404 | −11.767 | 18.116 | 1.00 | 20.06 | C |
| ATOM | 1798 | CB | ALA | L | 19 | 52.142 | −12.386 | 17.554 | 1.00 | 20.07 | C |
| ATOM | 1799 | C | ALA | L | 19 | 54.375 | −12.868 | 18.524 | 1.00 | 20.19 | C |
| ATOM | 1800 | O | ALA | L | 19 | 55.276 | −13.241 | 17.759 | 1.00 | 19.90 | O |
| ATOM | 1801 | N | SER | L | 20 | 54.170 | −13.396 | 19.725 | 1.00 | 20.61 | N |
| ATOM | 1802 | CA | SER | L | 20 | 55.048 | −14.401 | 20.287 | 1.00 | 20.84 | C |
| ATOM | 1803 | CB | SER | L | 20 | 56.150 | −13.695 | 21.082 | 1.00 | 21.67 | C |
| ATOM | 1804 | OG | SER | L | 20 | 57.002 | −14.624 | 21.695 | 1.00 | 24.81 | O |
| ATOM | 1805 | C | SER | L | 20 | 54.216 | −15.311 | 21.191 | 1.00 | 21.37 | C |
| ATOM | 1806 | O | SER | L | 20 | 53.359 | −14.843 | 21.937 | 1.00 | 20.65 | O |
| ATOM | 1807 | N | ILE | L | 21 | 54.476 | −16.610 | 21.111 | 1.00 | 21.24 | N |
| ATOM | 1808 | CA | ILE | L | 21 | 53.738 | −17.626 | 21.838 | 1.00 | 22.67 | C |
| ATOM | 1809 | CB | ILE | L | 21 | 52.772 | −18.351 | 20.878 | 1.00 | 23.46 | C |
| ATOM | 1810 | CG1 | ILE | L | 21 | 51.504 | −17.507 | 20.689 | 1.00 | 24.26 | C |
| ATOM | 1811 | CD1 | ILE | L | 21 | 50.862 | −17.717 | 19.342 | 1.00 | 30.23 | C |
| ATOM | 1812 | CG2 | ILE | L | 21 | 52.420 | −19.747 | 21.355 | 1.00 | 27.39 | C |
| ATOM | 1813 | C | ILE | L | 21 | 54.734 | −18.601 | 22.457 | 1.00 | 22.54 | C |
| ATOM | 1814 | O | ILE | L | 21 | 55.756 | −18.922 | 21.843 | 1.00 | 21.23 | O |
| ATOM | 1815 | N | SER | L | 22 | 54.410 | −19.089 | 23.654 | 1.00 | 22.03 | N |
| ATOM | 1816 | CA | SER | L | 22 | 55.350 | −19.887 | 24.428 | 1.00 | 22.70 | C |
| ATOM | 1817 | CB | SER | L | 22 | 55.653 | −19.163 | 25.747 | 1.00 | 23.31 | C |
| ATOM | 1818 | OG | SER | L | 22 | 56.395 | −19.985 | 26.621 | 1.00 | 25.19 | O |
| ATOM | 1819 | C | SER | L | 22 | 54.866 | −21.325 | 24.660 | 1.00 | 21.93 | C |
| ATOM | 1820 | O | SER | L | 22 | 53.680 | −21.566 | 24.839 | 1.00 | 21.04 | O |
| ATOM | 1821 | N | CYS | L | 23 | 55.814 | −22.263 | 24.665 | 1.00 | 22.22 | N |
| ATOM | 1822 | CA | CYS | L | 23 | 55.569 | −23.684 | 24.963 | 1.00 | 22.29 | C |
| ATOM | 1823 | CB | CYS | L | 23 | 55.582 | −24.532 | 23.678 | 1.00 | 22.99 | C |
| ATOM | 1824 | SG | CYS | L | 23 | 55.264 | −26.343 | 23.935 | 1.00 | 27.58 | S |
| ATOM | 1825 | C | CYS | L | 23 | 56.671 | −24.164 | 25.908 | 1.00 | 22.17 | C |
| ATOM | 1826 | O | CYS | L | 23 | 57.857 | −23.994 | 25.611 | 1.00 | 21.39 | O |
| ATOM | 1827 | N | THR | L | 24 | 56.280 | −24.725 | 27.049 | 1.00 | 21.28 | N |
| ATOM | 1828 | CA | THR | L | 24 | 57.205 | −25.329 | 28.009 | 1.00 | 22.17 | C |
| ATOM | 1829 | CB | THR | L | 24 | 57.063 | −24.652 | 29.397 | 1.00 | 22.52 | C |
| ATOM | 1830 | OG1 | THR | L | 24 | 57.449 | −23.279 | 29.283 | 1.00 | 25.17 | O |
| ATOM | 1831 | CG2 | THR | L | 24 | 57.964 | −25.322 | 30.453 | 1.00 | 24.44 | C |
| ATOM | 1832 | C | THR | L | 24 | 56.890 | −26.807 | 28.178 | 1.00 | 21.77 | C |
| ATOM | 1833 | O | THR | L | 24 | 55.731 | −27.161 | 28.436 | 1.00 | 22.23 | O |
| ATOM | 1834 | N | SER | L | 25 | 57.908 | −27.661 | 28.064 | 1.00 | 21.31 | N |
| ATOM | 1835 | CA | SER | L | 25 | 57.746 | −29.097 | 28.302 | 1.00 | 21.34 | C |
| ATOM | 1836 | CB | SER | L | 25 | 58.457 | −29.948 | 27.229 | 1.00 | 21.08 | C |
| ATOM | 1837 | OG | SER | L | 25 | 59.853 | −29.687 | 27.201 | 1.00 | 22.31 | O |
| ATOM | 1838 | C | SER | L | 25 | 58.231 | −29.493 | 29.682 | 1.00 | 20.69 | C |
| ATOM | 1839 | O | SER | L | 25 | 59.190 | −28.929 | 30.217 | 1.00 | 20.87 | O |
| ATOM | 1840 | N | GLY | L | 26 | 57.569 | −30.488 | 30.249 | 1.00 | 20.97 | N |
| ATOM | 1841 | CA | GLY | L | 26 | 57.874 | −30.953 | 31.596 | 1.00 | 21.45 | C |
| ATOM | 1842 | C | GLY | L | 26 | 59.171 | −31.725 | 31.643 | 1.00 | 21.34 | C |
| ATOM | 1843 | O | GLY | L | 26 | 59.666 | −32.022 | 32.723 | 1.00 | 22.20 | O |
| ATOM | 1844 | N | GLN | L | 27 | 59.712 | −32.080 | 30.481 | 1.00 | 21.10 | N |
| ATOM | 1845 | CA | GLN | L | 27 | 61.078 | −32.594 | 30.438 | 1.00 | 21.08 | C |
| ATOM | 1846 | CB | GLN | L | 27 | 61.145 | −34.098 | 30.763 | 1.00 | 22.33 | C |
| ATOM | 1847 | CG | GLN | L | 27 | 60.658 | −35.046 | 29.700 | 1.00 | 24.36 | C |
| ATOM | 1848 | CD | GLN | L | 27 | 60.904 | −36.504 | 30.078 | 1.00 | 26.65 | C |
| ATOM | 1849 | OE1 | GLN | L | 27 | 61.928 | −37.095 | 29.712 | 1.00 | 28.96 | O |
| ATOM | 1850 | NE2 | GLN | L | 27 | 59.983 | −37.080 | 30.850 | 1.00 | 26.73 | N |
| ATOM | 1851 | C | GLN | L | 27 | 61.780 | −32.229 | 29.128 | 1.00 | 20.18 | C |
| ATOM | 1852 | O | GLN | L | 27 | 61.135 | −31.851 | 28.139 | 1.00 | 19.56 | O |
| ATOM | 1853 | N | SER | L | 27A | 63.104 | −32.287 | 29.139 | 1.00 | 19.17 | N |
| ATOM | 1854 | CA | SER | L | 27A | 63.867 | −31.936 | 27.945 | 1.00 | 17.96 | C |
| ATOM | 1855 | CB | SER | L | 27A | 65.344 | −32.217 | 28.124 | 1.00 | 18.04 | C |
| ATOM | 1856 | OG | SER | L | 27A | 66.004 | −31.912 | 26.900 | 1.00 | 16.42 | O |
| ATOM | 1857 | C | SER | L | 27A | 63.372 | −32.690 | 26.715 | 1.00 | 17.89 | C |
| ATOM | 1858 | O | SER | L | 27A | 63.117 | −33.900 | 26.774 | 1.00 | 17.37 | O |
| ATOM | 1859 | N | LEU | L | 27B | 63.266 | −31.970 | 25.607 | 1.00 | 17.23 | N |
| ATOM | 1860 | CA | LEU | L | 27B | 62.897 | −32.583 | 24.327 | 1.00 | 18.19 | C |
| ATOM | 1861 | CB | LEU | L | 27B | 61.917 | −31.669 | 23.582 | 1.00 | 17.35 | C |
| ATOM | 1862 | CG | LEU | L | 27B | 60.611 | −31.397 | 24.342 | 1.00 | 17.33 | C |
| ATOM | 1863 | CD1 | LEU | L | 27B | 59.667 | −30.516 | 23.510 | 1.00 | 17.72 | C |
| ATOM | 1864 | CD2 | LEU | L | 27B | 59.914 | −32.708 | 24.727 | 1.00 | 17.44 | C |
| ATOM | 1865 | C | LEU | L | 27B | 64.124 | −32.924 | 23.466 | 1.00 | 18.10 | C |
| ATOM | 1866 | O | LEU | L | 27B | 63.993 | −33.227 | 22.291 | 1.00 | 19.49 | O |
| ATOM | 1867 | N | VAL | L | 27C | 65.317 | −32.869 | 24.056 | 1.00 | 18.50 | N |
| ATOM | 1868 | CA | VAL | L | 27C | 66.528 | −33.278 | 23.359 | 1.00 | 18.87 | C |
| ATOM | 1869 | CB | VAL | L | 27C | 67.841 | −32.729 | 24.015 | 1.00 | 19.66 | C |
| ATOM | 1870 | CG1 | VAL | L | 27C | 69.073 | −33.170 | 23.193 | 1.00 | 20.35 | C |
| ATOM | 1871 | CG2 | VAL | L | 27C | 67.808 | −31.199 | 24.152 | 1.00 | 18.31 | C |
| ATOM | 1872 | C | VAL | L | 27C | 66.561 | −34.799 | 23.356 | 1.00 | 18.81 | C |
| ATOM | 1873 | O | VAL | L | 27C | 66.645 | −35.434 | 24.412 | 1.00 | 18.07 | O |
| ATOM | 1874 | N | HIS | L | 27D | 66.489 | −35.361 | 22.155 | 1.00 | 18.63 | N |
| ATOM | 1875 | CA | HIS | L | 27D | 66.564 | −36.803 | 21.901 | 1.00 | 18.57 | C |
| ATOM | 1876 | CB | HIS | L | 27D | 66.117 | −37.059 | 20.456 | 1.00 | 18.26 | C |
| ATOM | 1877 | CG | HIS | L | 27D | 65.781 | −38.484 | 20.149 | 1.00 | 19.15 | C |
| ATOM | 1878 | ND1 | HIS | L | 27D | 66.739 | −39.425 | 19.831 | 1.00 | 19.41 | N |
| ATOM | 1879 | CE1 | HIS | L | 27D | 66.151 | −40.584 | 19.588 | 1.00 | 21.25 | C |
| ATOM | 1880 | NE2 | HIS | L | 27D | 64.844 | −40.424 | 19.713 | 1.00 | 22.01 | N |
| ATOM | 1881 | CD2 | HIS | L | 27D | 64.587 | −39.119 | 20.067 | 1.00 | 20.08 | C |
| ATOM | 1882 | C | HIS | L | 27D | 67.996 | −37.308 | 22.102 | 1.00 | 19.54 | C |
| ATOM | 1883 | O | HIS | L | 27D | 68.955 | −36.544 | 21.983 | 1.00 | 19.03 | O |
| ATOM | 1884 | N | ILE | L | 27E | 68.136 | −38.601 | 22.388 | 1.00 | 19.82 | N |
| ATOM | 1885 | CA | ILE | L | 27E | 69.457 | −39.220 | 22.497 | 1.00 | 21.12 | C |
| ATOM | 1886 | CB | ILE | L | 27E | 69.358 | −40.722 | 22.948 | 1.00 | 21.06 | C |
| ATOM | 1887 | CG1 | ILE | L | 27E | 70.698 | −41.233 | 23.488 | 1.00 | 24.04 | C |
| ATOM | 1888 | CD1 | ILE | L | 27E | 71.167 | −40.495 | 24.768 | 1.00 | 26.77 | C |
| ATOM | 1889 | CG2 | ILE | L | 27E | 68.829 | −41.612 | 21.828 | 1.00 | 21.40 | C |
| ATOM | 1890 | C | ILE | L | 27E | 70.275 | −39.036 | 21.202 | 1.00 | 21.27 | C |
| ATOM | 1891 | O | ILE | L | 27E | 71.506 | −38.956 | 21.256 | 1.00 | 20.97 | O |
| ATOM | 1892 | N | ASN | L | 28 | 69.589 | −38.933 | 20.057 | 1.00 | 20.44 | N |
| ATOM | 1893 | CA | ASN | L | 28 | 70.278 | −38.739 | 18.781 | 1.00 | 21.16 | C |
| ATOM | 1894 | CB | ASN | L | 28 | 69.454 | −39.293 | 17.594 | 1.00 | 19.94 | C |
| ATOM | 1895 | CG | ASN | L | 28 | 68.286 | −38.385 | 17.176 | 1.00 | 20.78 | C |
| ATOM | 1896 | OD1 | ASN | L | 28 | 68.114 | −37.269 | 17.675 | 1.00 | 19.81 | O |
| ATOM | 1897 | ND2 | ASN | L | 28 | 67.494 | −38.869 | 16.238 | 1.00 | 17.38 | N |
| ATOM | 1898 | C | ASN | L | 28 | 70.802 | −37.308 | 18.529 | 1.00 | 21.36 | C |
| ATOM | 1899 | O | ASN | L | 28 | 71.415 | −37.044 | 17.486 | 1.00 | 21.83 | O |
| ATOM | 1900 | N | GLY | L | 29 | 70.564 | −36.403 | 19.488 | 1.00 | 21.07 | N |
| ATOM | 1901 | CA | GLY | L | 29 | 71.013 | −35.010 | 19.405 | 1.00 | 21.20 | C |
| ATOM | 1902 | C | GLY | L | 29 | 69.999 | −33.986 | 18.889 | 1.00 | 21.32 | C |
| ATOM | 1903 | O | GLY | L | 29 | 70.186 | −32.768 | 19.070 | 1.00 | 21.35 | O |
| ATOM | 1904 | N | ASN | L | 30 | 68.928 | −34.454 | 18.252 | 1.00 | 20.67 | N |
| ATOM | 1905 | CA | ASN | L | 30 | 67.895 | −33.554 | 17.708 | 1.00 | 21.54 | C |
| ATOM | 1906 | CB | ASN | L | 30 | 67.232 | −34.153 | 16.444 | 1.00 | 21.67 | C |
| ATOM | 1907 | CG | ASN | L | 30 | 68.126 | −34.056 | 15.215 | 1.00 | 24.65 | C |
| ATOM | 1908 | OD1 | ASN | L | 30 | 69.084 | −33.274 | 15.190 | 1.00 | 27.70 | O |
| ATOM | 1909 | ND2 | ASN | L | 30 | 67.828 | −34.849 | 14.200 | 1.00 | 24.51 | N |
| ATOM | 1910 | C | ASN | L | 30 | 66.832 | −33.180 | 18.739 | 1.00 | 21.21 | C |
| ATOM | 1911 | O | ASN | L | 30 | 66.669 | −33.874 | 19.745 | 1.00 | 21.75 | O |
| ATOM | 1912 | N | THR | L | 31 | 66.107 | −32.090 | 18.490 | 1.00 | 20.94 | N |
| ATOM | 1913 | CA | THR | L | 31 | 65.026 | −31.691 | 19.398 | 1.00 | 20.05 | C |
| ATOM | 1914 | CB | THR | L | 31 | 65.311 | −30.301 | 20.030 | 1.00 | 19.52 | C |
| ATOM | 1915 | OG1 | THR | L | 31 | 66.473 | −30.382 | 20.866 | 1.00 | 19.29 | O |
| ATOM | 1916 | CG2 | THR | L | 31 | 64.141 | −29.841 | 20.886 | 1.00 | 19.13 | C |
| ATOM | 1917 | C | THR | L | 31 | 63.725 | −31.699 | 18.600 | 1.00 | 19.65 | C |
| ATOM | 1918 | O | THR | L | 31 | 63.538 | −30.842 | 17.742 | 1.00 | 20.66 | O |
| ATOM | 1919 | N | TYR | L | 32 | 62.839 | −32.662 | 18.858 | 1.00 | 18.70 | N |
| ATOM | 1920 | CA | TYR | L | 32 | 61.666 | −32.828 | 17.994 | 1.00 | 18.26 | C |
| ATOM | 1921 | CB | TYR | L | 32 | 61.328 | −34.303 | 17.783 | 1.00 | 17.37 | C |
| ATOM | 1922 | CG | TYR | L | 32 | 62.459 | −35.059 | 17.134 | 1.00 | 18.60 | C |
| ATOM | 1923 | CD1 | TYR | L | 32 | 62.625 | −35.032 | 15.742 | 1.00 | 17.28 | C |
| ATOM | 1924 | CE1 | TYR | L | 32 | 63.674 | −35.711 | 15.132 | 1.00 | 17.64 | C |
| ATOM | 1925 | CZ | TYR | L | 32 | 64.582 | −36.421 | 15.901 | 1.00 | 17.45 | C |
| ATOM | 1926 | OH | TYR | L | 32 | 65.599 | −37.106 | 15.264 | 1.00 | 16.35 | O |
| ATOM | 1927 | CE2 | TYR | L | 32 | 64.452 | −36.470 | 17.295 | 1.00 | 17.49 | C |
| ATOM | 1928 | CD2 | TYR | L | 32 | 63.391 | −35.769 | 17.908 | 1.00 | 17.64 | C |
| ATOM | 1929 | C | TYR | L | 32 | 60.470 | −32.058 | 18.554 | 1.00 | 18.01 | C |
| ATOM | 1930 | O | TYR | L | 32 | 59.512 | −32.644 | 19.067 | 1.00 | 17.37 | O |
| ATOM | 1931 | N | LEU | L | 33 | 60.574 | −30.737 | 18.484 | 1.00 | 17.60 | N |
| ATOM | 1932 | CA | LEU | L | 33 | 59.499 | −29.836 | 18.913 | 1.00 | 17.56 | C |
| ATOM | 1933 | CB | LEU | L | 33 | 60.022 | −28.812 | 19.954 | 1.00 | 17.45 | C |
| ATOM | 1934 | CG | LEU | L | 33 | 59.095 | −27.783 | 20.628 | 1.00 | 16.36 | C |
| ATOM | 1935 | CD1 | LEU | L | 33 | 57.766 | −28.404 | 21.121 | 1.00 | 17.29 | C |
| ATOM | 1936 | CD2 | LEU | L | 33 | 58.802 | −26.596 | 19.715 | 1.00 | 18.60 | C |
| ATOM | 1937 | C | LEU | L | 33 | 58.982 | −29.140 | 17.661 | 1.00 | 17.49 | C |
| ATOM | 1938 | O | LEU | L | 33 | 59.758 | −28.530 | 16.910 | 1.00 | 17.66 | O |
| ATOM | 1939 | N | HIS | L | 34 | 57.675 | −29.242 | 17.430 | 1.00 | 17.60 | N |
| ATOM | 1940 | CA | HIS | L | 34 | 57.041 | −28.681 | 16.237 | 1.00 | 17.26 | C |
| ATOM | 1941 | CB | HIS | L | 34 | 56.536 | −29.833 | 15.341 | 1.00 | 16.54 | C |
| ATOM | 1942 | CG | HIS | L | 34 | 57.397 | −31.064 | 15.410 | 1.00 | 17.09 | C |
| ATOM | 1943 | ND1 | HIS | L | 34 | 58.704 | −31.082 | 14.975 | 1.00 | 16.07 | N |
| ATOM | 1944 | CE1 | HIS | L | 34 | 59.221 | −32.283 | 15.179 | 1.00 | 17.52 | C |
| ATOM | 1945 | NE2 | HIS | L | 34 | 58.299 | −33.041 | 15.744 | 1.00 | 17.45 | N |
| ATOM | 1946 | CD2 | HIS | L | 34 | 57.150 | −32.301 | 15.900 | 1.00 | 15.81 | C |
| ATOM | 1947 | C | HIS | L | 34 | 55.869 | −27.774 | 16.616 | 1.00 | 18.03 | C |
| ATOM | 1948 | O | HIS | L | 34 | 55.331 | −27.887 | 17.716 | 1.00 | 17.99 | O |
| ATOM | 1949 | N | TRP | L | 35 | 55.477 | −26.900 | 15.690 | 1.00 | 17.86 | N |
| ATOM | 1950 | CA | TRP | L | 35 | 54.371 | −25.969 | 15.874 | 1.00 | 17.75 | C |
| ATOM | 1951 | CB | TRP | L | 35 | 54.895 | −24.539 | 15.821 | 1.00 | 17.29 | C |
| ATOM | 1952 | CG | TRP | L | 35 | 55.714 | −24.128 | 16.989 | 1.00 | 18.59 | C |
| ATOM | 1953 | CD1 | TRP | L | 35 | 57.076 | −24.150 | 17.093 | 1.00 | 19.62 | C |
| ATOM | 1954 | NE1 | TRP | L | 35 | 57.463 | −23.674 | 18.333 | 1.00 | 19.84 | N |
| ATOM | 1955 | CE2 | TRP | L | 35 | 56.337 | −23.364 | 19.053 | 1.00 | 19.80 | C |
| ATOM | 1956 | CD2 | TRP | L | 35 | 55.220 | −23.634 | 18.234 | 1.00 | 18.28 | C |
| ATOM | 1957 | CE3 | TRP | L | 35 | 53.934 | −23.390 | 18.736 | 1.00 | 18.35 | C |
| ATOM | 1958 | CZ3 | TRP | L | 35 | 53.804 | −22.882 | 20.006 | 1.00 | 22.06 | C |
| ATOM | 1959 | CH2 | TRP | L | 35 | 54.935 | −22.624 | 20.809 | 1.00 | 23.78 | C |
| ATOM | 1960 | CZ2 | TRP | L | 35 | 56.208 | −22.856 | 20.349 | 1.00 | 20.66 | C |
| ATOM | 1961 | C | TRP | L | 35 | 53.352 | −26.157 | 14.740 | 1.00 | 18.18 | C |
| ATOM | 1962 | O | TRP | L | 35 | 53.734 | −26.246 | 13.561 | 1.00 | 17.74 | O |
| ATOM | 1963 | N | TYR | L | 36 | 52.068 | −26.224 | 15.105 | 1.00 | 18.22 | N |
| ATOM | 1964 | CA | TYR | L | 36 | 50.980 | −26.450 | 14.155 | 1.00 | 18.36 | C |
| ATOM | 1965 | CB | TYR | L | 36 | 50.210 | −27.746 | 14.486 | 1.00 | 18.58 | C |
| ATOM | 1966 | CG | TYR | L | 36 | 50.953 | −28.983 | 14.079 | 1.00 | 18.33 | C |
| ATOM | 1967 | CD1 | TYR | L | 36 | 50.726 | −29.583 | 12.838 | 1.00 | 20.08 | C |
| ATOM | 1968 | CE1 | TYR | L | 36 | 51.456 | −30.723 | 12.446 | 1.00 | 17.84 | C |
| ATOM | 1969 | CZ | TYR | L | 36 | 52.393 | −31.238 | 13.315 | 1.00 | 18.26 | C |
| ATOM | 1970 | OH | TYR | L | 36 | 53.114 | −32.334 | 12.982 | 1.00 | 18.06 | O |
| ATOM | 1971 | CE2 | TYR | L | 36 | 52.617 | −30.666 | 14.557 | 1.00 | 19.70 | C |
| ATOM | 1972 | CD2 | TYR | L | 36 | 51.903 | −29.548 | 14.927 | 1.00 | 20.26 | C |
| ATOM | 1973 | C | TYR | L | 36 | 50.013 | −25.281 | 14.281 | 1.00 | 18.96 | C |
| ATOM | 1974 | O | TYR | L | 36 | 49.914 | −24.667 | 15.347 | 1.00 | 18.91 | O |
| ATOM | 1975 | N | LEU | L | 37 | 49.316 | −24.977 | 13.198 | 1.00 | 18.83 | N |
| ATOM | 1976 | CA | LEU | L | 37 | 48.210 | −24.031 | 13.251 | 1.00 | 19.78 | C |
| ATOM | 1977 | CB | LEU | L | 37 | 48.484 | −22.802 | 12.383 | 1.00 | 19.37 | C |
| ATOM | 1978 | CG | LEU | L | 37 | 47.319 | −21.829 | 12.167 | 1.00 | 19.56 | C |
| ATOM | 1979 | CD1 | LEU | L | 37 | 46.770 | −21.264 | 13.478 | 1.00 | 19.65 | C |
| ATOM | 1980 | CD2 | LEU | L | 37 | 47.732 | −20.700 | 11.249 | 1.00 | 18.30 | C |
| ATOM | 1981 | C | LEU | L | 37 | 46.941 | −24.724 | 12.785 | 1.00 | 19.60 | C |
| ATOM | 1982 | O | LEU | L | 37 | 46.914 | −25.293 | 11.711 | 1.00 | 19.24 | O |
| ATOM | 1983 | N | GLN | L | 38 | 45.889 | −24.643 | 13.596 | 1.00 | 20.95 | N |
| ATOM | 1984 | CA | GLN | L | 38 | 44.602 | −25.209 | 13.209 | 1.00 | 22.00 | C |
| ATOM | 1985 | CB | GLN | L | 38 | 44.189 | −26.312 | 14.194 | 1.00 | 22.08 | C |
| ATOM | 1986 | CG | GLN | L | 38 | 42.922 | −27.054 | 13.748 | 1.00 | 22.29 | C |
| ATOM | 1987 | CD | GLN | L | 38 | 42.443 | −28.048 | 14.759 | 1.00 | 22.81 | C |
| ATOM | 1988 | OE1 | GLN | L | 38 | 42.575 | −27.833 | 15.960 | 1.00 | 21.83 | O |
| ATOM | 1989 | NE2 | GLN | L | 38 | 41.898 | −29.167 | 14.279 | 1.00 | 20.60 | N |
| ATOM | 1990 | C | GLN | L | 38 | 43.483 | −24.157 | 13.074 | 1.00 | 23.41 | C |
| ATOM | 1991 | O | GLN | L | 38 | 43.256 | −23.332 | 13.968 | 1.00 | 23.24 | O |
| ATOM | 1992 | N | LYS | L | 39 | 42.783 | −24.203 | 11.951 | 1.00 | 24.94 | N |
| ATOM | 1993 | CA | LYS | L | 39 | 41.630 | −23.340 | 11.736 | 1.00 | 26.49 | C |
| ATOM | 1994 | CB | LYS | L | 39 | 41.672 | −22.782 | 10.323 | 1.00 | 26.80 | C |
| ATOM | 1995 | CG | LYS | L | 39 | 42.577 | −21.546 | 10.241 | 1.00 | 28.55 | C |
| ATOM | 1996 | CD | LYS | L | 39 | 43.510 | −21.610 | 9.071 | 1.00 | 33.19 | C |
| ATOM | 1997 | CE | LYS | L | 39 | 44.363 | −20.358 | 8.995 | 1.00 | 32.86 | C |
| ATOM | 1998 | NZ | LYS | L | 39 | 45.334 | −20.508 | 7.878 | 1.00 | 39.45 | N |
| ATOM | 1999 | C | LYS | L | 39 | 40.332 | −24.091 | 12.051 | 1.00 | 27.55 | C |
| ATOM | 2000 | O | LYS | L | 39 | 40.308 | −25.318 | 11.977 | 1.00 | 27.64 | O |
| ATOM | 2001 | N | PRO | L | 40 | 39.256 | −23.362 | 12.434 | 1.00 | 28.36 | N |
| ATOM | 2002 | CA | PRO | L | 40 | 38.045 | −24.079 | 12.848 | 1.00 | 28.71 | C |
| ATOM | 2003 | CB | PRO | L | 40 | 37.062 | −22.946 | 13.178 | 1.00 | 29.42 | C |
| ATOM | 2004 | CG | PRO | L | 40 | 37.957 | −21.791 | 13.578 | 1.00 | 28.98 | C |
| ATOM | 2005 | CD | PRO | L | 40 | 39.111 | −21.901 | 12.606 | 1.00 | 28.26 | C |
| ATOM | 2006 | C | PRO | L | 40 | 37.511 | −25.005 | 11.739 | 1.00 | 28.65 | C |
| ATOM | 2007 | O | PRO | L | 40 | 37.547 | −24.645 | 10.548 | 1.00 | 28.27 | O |
| ATOM | 2008 | N | GLY | L | 41 | 37.091 | −26.210 | 12.133 | 1.00 | 28.22 | N |
| ATOM | 2009 | CA | GLY | L | 41 | 36.560 | −27.206 | 11.184 | 1.00 | 28.12 | C |
| ATOM | 2010 | C | GLY | L | 41 | 37.600 | −27.936 | 10.329 | 1.00 | 28.16 | C |
| ATOM | 2011 | O | GLY | L | 41 | 37.250 | −28.792 | 9.514 | 1.00 | 28.53 | O |
| ATOM | 2012 | N | GLN | L | 42 | 38.882 | −27.615 | 10.511 | 1.00 | 27.56 | N |
| ATOM | 2013 | CA | GLN | L | 42 | 39.961 | −28.212 | 9.700 | 1.00 | 26.86 | C |
| ATOM | 2014 | CB | GLN | L | 42 | 40.763 | −27.121 | 9.008 | 1.00 | 27.35 | C |
| ATOM | 2015 | CG | GLN | L | 42 | 40.007 | −26.203 | 8.060 | 1.00 | 30.73 | C |
| ATOM | 2016 | CD | GLN | L | 42 | 40.923 | −25.122 | 7.500 | 1.00 | 34.52 | C |
| ATOM | 2017 | OE1 | GLN | L | 42 | 42.155 | −25.196 | 7.636 | 1.00 | 37.43 | O |
| ATOM | 2018 | NE2 | GLN | L | 42 | 40.330 | −24.104 | 6.883 | 1.00 | 35.98 | N |
| ATOM | 2019 | C | GLN | L | 42 | 40.935 | −29.008 | 10.574 | 1.00 | 25.73 | C |
| ATOM | 2020 | O | GLN | L | 42 | 40.935 | −28.847 | 11.801 | 1.00 | 25.25 | O |
| ATOM | 2021 | N | SER | L | 43 | 41.764 | −29.852 | 9.950 | 1.00 | 23.96 | N |
| ATOM | 2022 | CA | SER | L | 43 | 42.858 | −30.518 | 10.673 | 1.00 | 22.71 | C |
| ATOM | 2023 | CB | SER | L | 43 | 43.347 | −31.747 | 9.898 | 1.00 | 22.69 | C |
| ATOM | 2024 | OG | SER | L | 43 | 43.785 | −31.396 | 8.598 | 1.00 | 23.13 | O |
| ATOM | 2025 | C | SER | L | 43 | 44.007 | −29.522 | 10.913 | 1.00 | 21.43 | C |
| ATOM | 2026 | O | SER | L | 43 | 44.082 | −28.511 | 10.218 | 1.00 | 21.16 | O |
| ATOM | 2027 | N | PRO | L | 44 | 44.897 | −29.792 | 11.899 | 1.00 | 20.56 | N |
| ATOM | 2028 | CA | PRO | L | 44 | 46.051 | −28.920 | 12.110 | 1.00 | 20.37 | C |
| ATOM | 2029 | CB | PRO | L | 44 | 46.742 | −29.548 | 13.335 | 1.00 | 20.14 | C |
| ATOM | 2030 | CG | PRO | L | 44 | 45.629 | −30.306 | 14.039 | 1.00 | 20.54 | C |
| ATOM | 2031 | CD | PRO | L | 44 | 44.857 | −30.880 | 12.894 | 1.00 | 20.34 | C |
| ATOM | 2032 | C | PRO | L | 44 | 46.989 | −28.957 | 10.898 | 1.00 | 20.59 | C |
| ATOM | 2033 | O | PRO | L | 44 | 47.027 | −29.963 | 10.171 | 1.00 | 20.11 | O |
| ATOM | 2034 | N | LYS | L | 45 | 47.723 | −27.870 | 10.682 | 1.00 | 20.40 | N |
| ATOM | 2035 | CA | LYS | L | 45 | 48.727 | −27.806 | 9.613 | 1.00 | 20.25 | C |
| ATOM | 2036 | CB | LYS | L | 45 | 48.327 | −26.776 | 8.543 | 1.00 | 22.02 | C |
| ATOM | 2037 | CG | LYS | L | 45 | 47.146 | −27.214 | 7.673 | 1.00 | 23.81 | C |
| ATOM | 2038 | CD | LYS | L | 45 | 46.724 | −26.138 | 6.637 | 1.00 | 30.04 | C |
| ATOM | 2039 | CE | LYS | L | 45 | 47.584 | −26.175 | 5.364 | 1.00 | 33.83 | C |
| ATOM | 2040 | NZ | LYS | L | 45 | 47.620 | −27.521 | 4.657 | 1.00 | 36.12 | N |
| ATOM | 2041 | C | LYS | L | 45 | 50.083 | −27.444 | 10.193 | 1.00 | 19.97 | C |
| ATOM | 2042 | O | LYS | L | 45 | 50.189 | −26.558 | 11.050 | 1.00 | 19.10 | O |
| ATOM | 2043 | N | LEU | L | 46 | 51.116 | −28.137 | 9.726 | 1.00 | 19.18 | N |
| ATOM | 2044 | CA | LEU | L | 46 | 52.466 | −27.990 | 10.266 | 1.00 | 19.23 | C |
| ATOM | 2045 | CB | LEU | L | 46 | 53.336 | −29.178 | 9.816 | 1.00 | 19.25 | C |
| ATOM | 2046 | CG | LEU | L | 46 | 54.808 | −29.256 | 10.238 | 1.00 | 19.20 | C |
| ATOM | 2047 | CD1 | LEU | L | 46 | 55.004 | −29.137 | 11.761 | 1.00 | 15.36 | C |
| ATOM | 2048 | CD2 | LEU | L | 46 | 55.402 | −30.575 | 9.685 | 1.00 | 20.66 | C |
| ATOM | 2049 | C | LEU | L | 46 | 53.101 | −26.673 | 9.822 | 1.00 | 18.70 | C |
| ATOM | 2050 | O | LEU | L | 46 | 53.114 | −26.354 | 8.638 | 1.00 | 17.97 | O |
| ATOM | 2051 | N | LEU | L | 47 | 53.623 | −25.914 | 10.778 | 1.00 | 17.72 | N |
| ATOM | 2052 | CA | LEU | L | 47 | 54.295 | −24.671 | 10.442 | 1.00 | 18.17 | C |
| ATOM | 2053 | CB | LEU | L | 47 | 53.747 | −23.500 | 11.270 | 1.00 | 17.84 | C |
| ATOM | 2054 | CG | LEU | L | 47 | 52.254 | −23.120 | 11.222 | 1.00 | 20.33 | C |
| ATOM | 2055 | CD1 | LEU | L | 47 | 52.004 | −21.905 | 12.165 | 1.00 | 21.19 | C |
| ATOM | 2056 | CD2 | LEU | L | 47 | 51.753 | −22.784 | 9.819 | 1.00 | 20.92 | C |
| ATOM | 2057 | C | LEU | L | 47 | 55.801 | −24.798 | 10.577 | 1.00 | 17.59 | C |
| ATOM | 2058 | O | LEU | L | 47 | 56.553 | −24.432 | 9.656 | 1.00 | 16.63 | O |
| ATOM | 2059 | N | ILE | L | 48 | 56.235 | −25.355 | 11.708 | 1.00 | 17.89 | N |
| ATOM | 2060 | CA | ILE | L | 48 | 57.643 | −25.387 | 12.064 | 1.00 | 17.86 | C |
| ATOM | 2061 | CB | ILE | L | 48 | 57.975 | −24.318 | 13.140 | 1.00 | 17.74 | C |
| ATOM | 2062 | CG1 | ILE | L | 48 | 57.598 | −22.927 | 12.620 | 1.00 | 19.25 | C |
| ATOM | 2063 | CD1 | ILE | L | 48 | 57.959 | −21.771 | 13.551 | 1.00 | 21.07 | C |
| ATOM | 2064 | CG2 | ILE | L | 48 | 59.451 | −24.397 | 13.532 | 1.00 | 18.94 | C |
| ATOM | 2065 | C | ILE | L | 48 | 57.962 | −26.748 | 12.629 | 1.00 | 17.85 | C |
| ATOM | 2066 | O | ILE | L | 48 | 57.245 | −27.222 | 13.510 | 1.00 | 17.06 | O |
| ATOM | 2067 | N | TYR | L | 49 | 59.027 | −27.374 | 12.116 | 1.00 | 17.27 | N |
| ATOM | 2068 | CA | TYR | L | 49 | 59.498 | −28.643 | 12.664 | 1.00 | 18.12 | C |
| ATOM | 2069 | CB | TYR | L | 49 | 59.422 | −29.766 | 11.615 | 1.00 | 17.48 | C |
| ATOM | 2070 | CG | TYR | L | 49 | 60.384 | −29.615 | 10.447 | 1.00 | 19.15 | C |
| ATOM | 2071 | CD1 | TYR | L | 49 | 61.674 | −30.150 | 10.518 | 1.00 | 21.15 | C |
| ATOM | 2072 | CE1 | TYR | L | 49 | 62.572 | −30.031 | 9.453 | 1.00 | 21.98 | C |
| ATOM | 2073 | CZ | TYR | L | 49 | 62.175 | −29.381 | 8.296 | 1.00 | 24.76 | C |
| ATOM | 2074 | OH | TYR | L | 49 | 63.073 | −29.262 | 7.285 | 1.00 | 26.22 | O |
| ATOM | 2075 | CE2 | TYR | L | 49 | 60.907 | −28.834 | 8.189 | 1.00 | 22.57 | C |
| ATOM | 2076 | CD2 | TYR | L | 49 | 60.010 | −28.955 | 9.273 | 1.00 | 21.83 | C |
| ATOM | 2077 | C | TYR | L | 49 | 60.889 | −28.533 | 13.287 | 1.00 | 18.62 | C |
| ATOM | 2078 | O | TYR | L | 49 | 61.707 | −27.720 | 12.849 | 1.00 | 19.13 | O |
| ATOM | 2079 | N | LYS | L | 50 | 61.152 | −29.363 | 14.295 | 1.00 | 19.11 | N |
| ATOM | 2080 | CA | LYS | L | 50 | 62.435 | −29.371 | 15.005 | 1.00 | 19.36 | C |
| ATOM | 2081 | CB | LYS | L | 50 | 63.537 | −30.048 | 14.165 | 1.00 | 19.72 | C |
| ATOM | 2082 | CG | LYS | L | 50 | 63.477 | −31.583 | 14.144 | 1.00 | 19.25 | C |
| ATOM | 2083 | CD | LYS | L | 50 | 64.804 | −32.159 | 13.614 | 1.00 | 20.51 | C |
| ATOM | 2084 | CE | LYS | L | 50 | 64.951 | −31.886 | 12.133 | 1.00 | 20.38 | C |
| ATOM | 2085 | NZ | LYS | L | 50 | 66.094 | −32.660 | 11.532 | 1.00 | 23.67 | N |
| ATOM | 2086 | C | LYS | L | 50 | 62.850 | −27.958 | 15.411 | 1.00 | 19.08 | C |
| ATOM | 2087 | O | LYS | L | 50 | 63.882 | −27.452 | 14.980 | 1.00 | 18.18 | O |
| ATOM | 2088 | N | VAL | L | 51 | 61.981 | −27.320 | 16.192 | 1.00 | 19.67 | N |
| ATOM | 2089 | CA | VAL | L | 51 | 62.220 | −26.018 | 16.827 | 1.00 | 18.98 | C |
| ATOM | 2090 | CB | VAL | L | 51 | 63.552 | −25.967 | 17.625 | 1.00 | 19.72 | C |
| ATOM | 2091 | CG1 | VAL | L | 51 | 63.708 | −24.616 | 18.361 | 1.00 | 18.64 | C |
| ATOM | 2092 | CG2 | VAL | L | 51 | 63.646 | −27.142 | 18.607 | 1.00 | 20.32 | C |
| ATOM | 2093 | C | VAL | L | 51 | 62.167 | −24.826 | 15.886 | 1.00 | 19.30 | C |
| ATOM | 2094 | O | VAL | L | 51 | 61.493 | −23.851 | 16.205 | 1.00 | 19.64 | O |
| ATOM | 2095 | N | SER | L | 52 | 62.860 | −24.897 | 14.746 | 1.00 | 19.54 | N |
| ATOM | 2096 | CA | SER | L | 52 | 63.180 | −23.691 | 13.956 | 1.00 | 19.86 | C |
| ATOM | 2097 | CB | SER | L | 52 | 64.551 | −23.120 | 14.368 | 1.00 | 20.04 | C |
| ATOM | 2098 | OG | SER | L | 52 | 65.579 | −24.024 | 14.055 | 1.00 | 20.71 | O |
| ATOM | 2099 | C | SER | L | 52 | 63.117 | −23.838 | 12.422 | 1.00 | 19.94 | C |
| ATOM | 2100 | O | SER | L | 52 | 63.315 | −22.852 | 11.685 | 1.00 | 20.10 | O |
| ATOM | 2101 | N | ASN | L | 53 | 62.828 | −25.040 | 11.934 | 1.00 | 19.23 | N |
| ATOM | 2102 | CA | ASN | L | 53 | 62.781 | −25.268 | 10.497 | 1.00 | 19.90 | C |
| ATOM | 2103 | CB | ASN | L | 53 | 63.171 | −26.705 | 10.164 | 1.00 | 20.03 | C |
| ATOM | 2104 | CG | ASN | L | 53 | 64.550 | −27.053 | 10.647 | 1.00 | 20.66 | C |
| ATOM | 2105 | OD1 | ASN | L | 53 | 64.715 | −27.569 | 11.744 | 1.00 | 24.42 | O |
| ATOM | 2106 | ND2 | ASN | L | 53 | 65.544 | −26.770 | 9.839 | 1.00 | 19.16 | N |
| ATOM | 2107 | C | ASN | L | 53 | 61.401 | −24.999 | 9.926 | 1.00 | 19.61 | C |
| ATOM | 2108 | O | ASN | L | 53 | 60.427 | −25.565 | 10.401 | 1.00 | 19.65 | O |
| ATOM | 2109 | N | LEU | L | 54 | 61.323 | −24.157 | 8.901 | 1.00 | 19.75 | N |
| ATOM | 2110 | CA | LEU | L | 54 | 60.028 | −23.808 | 8.324 | 1.00 | 20.49 | C |
| ATOM | 2111 | CB | LEU | L | 54 | 60.120 | −22.525 | 7.511 | 1.00 | 21.15 | C |
| ATOM | 2112 | CG | LEU | L | 54 | 60.245 | −21.194 | 8.248 | 1.00 | 22.36 | C |
| ATOM | 2113 | CD1 | LEU | L | 54 | 60.019 | −20.056 | 7.238 | 1.00 | 23.34 | C |
| ATOM | 2114 | CD2 | LEU | L | 54 | 59.224 | −21.113 | 9.395 | 1.00 | 24.60 | C |
| ATOM | 2115 | C | LEU | L | 54 | 59.564 | −24.928 | 7.419 | 1.00 | 20.41 | C |
| ATOM | 2116 | O | LEU | L | 54 | 60.322 | −25.403 | 6.587 | 1.00 | 19.04 | O |
| ATOM | 2117 | N | PHE | L | 55 | 58.313 | −25.338 | 7.575 | 1.00 | 20.42 | N |
| ATOM | 2118 | CA | PHE | L | 55 | 57.768 | −26.359 | 6.687 | 1.00 | 21.58 | C |
| ATOM | 2119 | CB | PHE | L | 55 | 56.485 | −26.944 | 7.272 | 1.00 | 21.67 | C |
| ATOM | 2120 | CG | PHE | L | 55 | 55.949 | −28.143 | 6.511 | 1.00 | 23.44 | C |
| ATOM | 2121 | CD1 | PHE | L | 55 | 56.742 | −29.274 | 6.320 | 1.00 | 24.23 | C |
| ATOM | 2122 | CE1 | PHE | L | 55 | 56.241 | −30.383 | 5.643 | 1.00 | 25.62 | C |
| ATOM | 2123 | CZ | PHE | L | 55 | 54.939 | −30.372 | 5.150 | 1.00 | 24.60 | C |
| ATOM | 2124 | CE2 | PHE | L | 55 | 54.126 | −29.251 | 5.345 | 1.00 | 25.89 | C |
| ATOM | 2125 | CD2 | PHE | L | 55 | 54.638 | −28.145 | 6.028 | 1.00 | 24.08 | C |
| ATOM | 2126 | C | PHE | L | 55 | 57.526 | −25.735 | 5.325 | 1.00 | 21.84 | C |
| ATOM | 2127 | O | PHE | L | 55 | 57.257 | −24.533 | 5.225 | 1.00 | 21.11 | O |
| ATOM | 2128 | N | SER | L | 56 | 57.630 | −26.536 | 4.270 | 1.00 | 22.30 | N |
| ATOM | 2129 | CA | SER | L | 56 | 57.532 | −25.996 | 2.919 | 1.00 | 22.71 | C |
| ATOM | 2130 | CB | SER | L | 56 | 57.758 | −27.090 | 1.867 | 1.00 | 23.15 | C |
| ATOM | 2131 | OG | SER | L | 56 | 57.414 | −26.605 | 0.586 | 1.00 | 25.04 | O |
| ATOM | 2132 | C | SER | L | 56 | 56.210 | −25.261 | 2.691 | 1.00 | 22.96 | C |
| ATOM | 2133 | O | SER | L | 56 | 55.128 | −25.762 | 3.031 | 1.00 | 22.83 | O |
| ATOM | 2134 | N | GLY | L | 57 | 56.311 | −24.056 | 2.128 | 1.00 | 22.76 | N |
| ATOM | 2135 | CA | GLY | L | 57 | 55.151 | −23.196 | 1.911 | 1.00 | 21.95 | C |
| ATOM | 2136 | C | GLY | L | 57 | 54.883 | −22.190 | 3.026 | 1.00 | 21.73 | C |
| ATOM | 2137 | O | GLY | L | 57 | 54.113 | −21.240 | 2.827 | 1.00 | 20.60 | O |
| ATOM | 2138 | N | VAL | L | 58 | 55.503 | −22.378 | 4.199 | 1.00 | 20.58 | N |
| ATOM | 2139 | CA | VAL | L | 58 | 55.240 | −21.484 | 5.336 | 1.00 | 20.26 | C |
| ATOM | 2140 | CB | VAL | L | 58 | 55.548 | −22.175 | 6.696 | 1.00 | 20.84 | C |
| ATOM | 2141 | CG1 | VAL | L | 58 | 55.406 | −21.198 | 7.874 | 1.00 | 19.89 | C |
| ATOM | 2142 | CG2 | VAL | L | 58 | 54.636 | −23.392 | 6.887 | 1.00 | 19.62 | C |
| ATOM | 2143 | C | VAL | L | 58 | 56.060 | −20.189 | 5.153 | 1.00 | 20.28 | C |
| ATOM | 2144 | O | VAL | L | 58 | 57.268 | −20.258 | 4.946 | 1.00 | 20.07 | O |
| ATOM | 2145 | N | PRO | L | 59 | 55.408 | −19.009 | 5.224 | 1.00 | 19.86 | N |
| ATOM | 2146 | CA | PRO | L | 59 | 56.186 | −17.770 | 5.004 | 1.00 | 19.37 | C |
| ATOM | 2147 | CB | PRO | L | 59 | 55.109 | −16.671 | 4.918 | 1.00 | 19.54 | C |
| ATOM | 2148 | CG | PRO | L | 59 | 53.897 | −17.234 | 5.597 | 1.00 | 20.49 | C |
| ATOM | 2149 | CD | PRO | L | 59 | 53.974 | −18.751 | 5.471 | 1.00 | 20.22 | C |
| ATOM | 2150 | C | PRO | L | 59 | 57.204 | −17.453 | 6.115 | 1.00 | 18.90 | C |
| ATOM | 2151 | O | PRO | L | 59 | 56.973 | −17.773 | 7.294 | 1.00 | 17.99 | O |
| ATOM | 2152 | N | ASP | L | 60 | 58.303 | −16.800 | 5.737 | 1.00 | 19.06 | N |
| ATOM | 2153 | CA | ASP | L | 60 | 59.393 | −16.484 | 6.680 | 1.00 | 18.39 | C |
| ATOM | 2154 | CB | ASP | L | 60 | 60.729 | −16.219 | 5.947 | 1.00 | 18.80 | C |
| ATOM | 2155 | CG | ASP | L | 60 | 60.728 | −14.945 | 5.092 | 1.00 | 20.07 | C |
| ATOM | 2156 | OD1 | ASP | L | 60 | 59.884 | −14.043 | 5.292 | 1.00 | 19.22 | O |
| ATOM | 2157 | OD2 | ASP | L | 60 | 61.602 | −14.866 | 4.199 | 1.00 | 17.73 | O |
| ATOM | 2158 | C | ASP | L | 60 | 59.091 | −15.379 | 7.702 | 1.00 | 18.13 | C |
| ATOM | 2159 | O | ASP | L | 60 | 59.982 | −14.958 | 8.431 | 1.00 | 18.11 | O |
| ATOM | 2160 | N | ARG | L | 61 | 57.853 | −14.879 | 7.740 | 1.00 | 17.40 | N |
| ATOM | 2161 | CA | ARG | L | 61 | 57.428 | −14.119 | 8.911 | 1.00 | 17.46 | C |
| ATOM | 2162 | CB | ARG | L | 61 | 56.186 | −13.261 | 8.638 | 1.00 | 18.05 | C |
| ATOM | 2163 | CG | ARG | L | 61 | 54.900 | −14.007 | 8.545 | 1.00 | 20.68 | C |
| ATOM | 2164 | CD | ARG | L | 61 | 53.686 | −13.026 | 8.446 | 1.00 | 22.91 | C |
| ATOM | 2165 | NE | ARG | L | 61 | 52.466 | −13.793 | 8.269 | 1.00 | 23.13 | N |
| ATOM | 2166 | CZ | ARG | L | 61 | 52.058 | −14.251 | 7.093 | 1.00 | 24.40 | C |
| ATOM | 2167 | NH1 | ARG | L | 61 | 52.752 | −13.970 | 5.984 | 1.00 | 25.84 | N |
| ATOM | 2168 | NH2 | ARG | L | 61 | 50.952 | −14.965 | 7.017 | 1.00 | 24.02 | N |
| ATOM | 2169 | C | ARG | L | 61 | 57.261 | −14.993 | 10.164 | 1.00 | 17.24 | C |
| ATOM | 2170 | O | ARG | L | 61 | 57.240 | −14.479 | 11.279 | 1.00 | 16.43 | O |
| ATOM | 2171 | N | PHE | L | 62 | 57.137 | −16.310 | 9.980 | 1.00 | 16.68 | N |
| ATOM | 2172 | CA | PHE | L | 62 | 57.168 | −17.250 | 11.108 | 1.00 | 17.27 | C |
| ATOM | 2173 | CB | PHE | L | 62 | 56.351 | −18.507 | 10.811 | 1.00 | 17.49 | C |
| ATOM | 2174 | CG | PHE | L | 62 | 54.885 | −18.261 | 10.789 | 1.00 | 17.52 | C |
| ATOM | 2175 | CD1 | PHE | L | 62 | 54.125 | −18.389 | 11.954 | 1.00 | 19.64 | C |
| ATOM | 2176 | CE1 | PHE | L | 62 | 52.744 | −18.133 | 11.938 | 1.00 | 19.59 | C |
| ATOM | 2177 | CZ | PHE | L | 62 | 52.141 | −17.739 | 10.757 | 1.00 | 20.07 | C |
| ATOM | 2178 | CE2 | PHE | L | 62 | 52.902 | −17.609 | 9.592 | 1.00 | 19.74 | C |
| ATOM | 2179 | CD2 | PHE | L | 62 | 54.266 | −17.875 | 9.623 | 1.00 | 18.03 | C |
| ATOM | 2180 | C | PHE | L | 62 | 58.593 | −17.649 | 11.433 | 1.00 | 17.72 | C |
| ATOM | 2181 | O | PHE | L | 62 | 59.392 | −17.825 | 10.535 | 1.00 | 17.26 | O |
| ATOM | 2182 | N | SER | L | 63 | 58.902 | −17.753 | 12.727 | 1.00 | 18.21 | N |
| ATOM | 2183 | CA | SER | L | 63 | 60.157 | −18.329 | 13.200 | 1.00 | 18.59 | C |
| ATOM | 2184 | CB | SER | L | 63 | 61.272 | −17.288 | 13.347 | 1.00 | 19.43 | C |
| ATOM | 2185 | OG | SER | L | 63 | 60.925 | −16.271 | 14.256 | 1.00 | 20.38 | O |
| ATOM | 2186 | C | SER | L | 63 | 59.924 | −19.064 | 14.517 | 1.00 | 18.71 | C |
| ATOM | 2187 | O | SER | L | 63 | 58.923 | −18.842 | 15.201 | 1.00 | 18.91 | O |
| ATOM | 2188 | N | GLY | L | 64 | 60.830 | −19.973 | 14.829 | 1.00 | 17.90 | N |
| ATOM | 2189 | CA | GLY | L | 64 | 60.761 | −20.726 | 16.070 | 1.00 | 18.67 | C |
| ATOM | 2190 | C | GLY | L | 64 | 62.114 | −20.711 | 16.729 | 1.00 | 18.31 | C |
| ATOM | 2191 | O | GLY | L | 64 | 63.139 | −20.743 | 16.047 | 1.00 | 17.95 | O |
| ATOM | 2192 | N | SER | L | 65 | 62.120 | −20.708 | 18.058 | 1.00 | 19.08 | N |
| ATOM | 2193 | CA | SER | L | 65 | 63.371 | −20.726 | 18.797 | 1.00 | 19.99 | C |
| ATOM | 2194 | CB | SER | L | 65 | 63.832 | −19.289 | 19.081 | 1.00 | 20.62 | C |
| ATOM | 2195 | OG | SER | L | 65 | 62.788 | −18.599 | 19.750 | 1.00 | 22.33 | O |
| ATOM | 2196 | C | SER | L | 65 | 63.196 | −21.456 | 20.107 | 1.00 | 20.38 | C |
| ATOM | 2197 | O | SER | L | 65 | 62.079 | −21.880 | 20.462 | 1.00 | 19.76 | O |
| ATOM | 2198 | N | GLY | L | 66 | 64.306 | −21.608 | 20.824 | 1.00 | 20.10 | N |
| ATOM | 2199 | CA | GLY | L | 66 | 64.264 | −22.171 | 22.164 | 1.00 | 20.66 | C |
| ATOM | 2200 | C | GLY | L | 66 | 65.107 | −23.424 | 22.252 | 1.00 | 21.31 | C |
| ATOM | 2201 | O | GLY | L | 66 | 65.705 | −23.845 | 21.272 | 1.00 | 20.26 | O |
| ATOM | 2202 | N | SER | L | 67 | 65.146 | −24.028 | 23.436 | 1.00 | 21.84 | N |
| ATOM | 2203 | CA | SER | L | 67 | 65.921 | −25.255 | 23.613 | 1.00 | 21.85 | C |
| ATOM | 2204 | CB | SER | L | 67 | 67.415 | −24.954 | 23.551 | 1.00 | 22.48 | C |
| ATOM | 2205 | OG | SER | L | 67 | 67.763 | −24.112 | 24.600 | 1.00 | 25.25 | O |
| ATOM | 2206 | C | SER | L | 67 | 65.605 | −25.936 | 24.932 | 1.00 | 21.05 | C |
| ATOM | 2207 | O | SER | L | 67 | 65.222 | −25.273 | 25.912 | 1.00 | 21.86 | O |
| ATOM | 2208 | N | GLY | L | 68 | 65.747 | −27.261 | 24.935 | 1.00 | 20.34 | N |
| ATOM | 2209 | CA | GLY | L | 68 | 65.591 | −28.088 | 26.132 | 1.00 | 19.64 | C |
| ATOM | 2210 | C | GLY | L | 68 | 64.118 | −28.219 | 26.460 | 1.00 | 19.66 | C |
| ATOM | 2211 | O | GLY | L | 68 | 63.438 | −29.129 | 25.978 | 1.00 | 18.48 | O |
| ATOM | 2212 | N | THR | L | 69 | 63.630 | −27.282 | 27.271 | 1.00 | 18.86 | N |
| ATOM | 2213 | CA | THR | L | 69 | 62.267 | −27.316 | 27.765 | 1.00 | 19.61 | C |
| ATOM | 2214 | CB | THR | L | 69 | 62.281 | −27.475 | 29.304 | 1.00 | 19.92 | C |
| ATOM | 2215 | OG1 | THR | L | 69 | 63.156 | −26.485 | 29.851 | 1.00 | 19.20 | O |
| ATOM | 2216 | CG2 | THR | L | 69 | 62.806 | −28.876 | 29.706 | 1.00 | 18.98 | C |
| ATOM | 2217 | C | THR | L | 69 | 61.449 | −26.048 | 27.426 | 1.00 | 19.94 | C |
| ATOM | 2218 | O | THR | L | 69 | 60.223 | −26.018 | 27.652 | 1.00 | 20.34 | O |
| ATOM | 2219 | N | ASP | L | 70 | 62.101 | −25.022 | 26.888 | 1.00 | 19.82 | N |
| ATOM | 2220 | CA | ASP | L | 70 | 61.459 | −23.705 | 26.694 | 1.00 | 20.91 | C |
| ATOM | 2221 | CB | ASP | L | 70 | 62.163 | −22.635 | 27.523 | 1.00 | 22.58 | C |
| ATOM | 2222 | CG | ASP | L | 70 | 62.168 | −22.943 | 29.021 | 1.00 | 28.30 | C |
| ATOM | 2223 | OD1 | ASP | L | 70 | 61.271 | −23.657 | 29.530 | 1.00 | 34.94 | O |
| ATOM | 2224 | OD2 | ASP | L | 70 | 63.079 | −22.439 | 29.708 | 1.00 | 34.66 | O |
| ATOM | 2225 | C | ASP | L | 70 | 61.516 | −23.297 | 25.227 | 1.00 | 20.01 | C |
| ATOM | 2226 | O | ASP | L | 70 | 62.608 | −23.166 | 24.675 | 1.00 | 18.73 | O |
| ATOM | 2227 | N | PHE | L | 71 | 60.350 | −23.084 | 24.604 | 1.00 | 18.46 | N |
| ATOM | 2228 | CA | PHE | L | 71 | 60.263 | −22.876 | 23.142 | 1.00 | 17.80 | C |
| ATOM | 2229 | CB | PHE | L | 71 | 59.805 | −24.168 | 22.432 | 1.00 | 18.19 | C |
| ATOM | 2230 | CG | PHE | L | 71 | 60.714 | −25.333 | 22.693 | 1.00 | 16.87 | C |
| ATOM | 2231 | CD1 | PHE | L | 71 | 61.871 | −25.506 | 21.934 | 1.00 | 17.68 | C |
| ATOM | 2232 | CE1 | PHE | L | 71 | 62.768 | −26.563 | 22.222 | 1.00 | 18.28 | C |
| ATOM | 2233 | CZ | PHE | L | 71 | 62.492 | −27.437 | 23.278 | 1.00 | 19.30 | C |
| ATOM | 2234 | CE2 | PHE | L | 71 | 61.326 | −27.260 | 24.055 | 1.00 | 16.99 | C |
| ATOM | 2235 | CD2 | PHE | L | 71 | 60.450 | −26.215 | 23.749 | 1.00 | 17.85 | C |
| ATOM | 2236 | C | PHE | L | 71 | 59.321 | −21.727 | 22.818 | 1.00 | 17.95 | C |
| ATOM | 2237 | O | PHE | L | 71 | 58.354 | −21.487 | 23.551 | 1.00 | 16.35 | O |
| ATOM | 2238 | N | THR | L | 72 | 59.617 | −21.031 | 21.719 | 1.00 | 17.79 | N |
| ATOM | 2239 | CA | THR | L | 72 | 58.870 | −19.839 | 21.328 | 1.00 | 19.53 | C |
| ATOM | 2240 | CB | THR | L | 72 | 59.668 | −18.585 | 21.741 | 1.00 | 19.72 | C |
| ATOM | 2241 | OG1 | THR | L | 72 | 59.951 | −18.670 | 23.142 | 1.00 | 22.13 | O |
| ATOM | 2242 | CG2 | THR | L | 72 | 58.878 | −17.311 | 21.480 | 1.00 | 22.17 | C |
| ATOM | 2243 | C | THR | L | 72 | 58.597 | −19.819 | 19.834 | 1.00 | 19.38 | C |
| ATOM | 2244 | O | THR | L | 72 | 59.512 | −20.009 | 19.033 | 1.00 | 19.94 | O |
| ATOM | 2245 | N | LEU | L | 73 | 57.334 | −19.606 | 19.463 | 1.00 | 18.96 | N |
| ATOM | 2246 | CA | LEU | L | 73 | 56.994 | −19.299 | 18.080 | 1.00 | 19.02 | C |
| ATOM | 2247 | CB | LEU | L | 73 | 55.751 | −20.078 | 17.630 | 1.00 | 19.14 | C |
| ATOM | 2248 | CG | LEU | L | 73 | 55.028 | −19.748 | 16.305 | 1.00 | 22.74 | C |
| ATOM | 2249 | CD1 | LEU | L | 73 | 55.958 | −19.507 | 15.169 | 1.00 | 28.87 | C |
| ATOM | 2250 | CD2 | LEU | L | 73 | 54.119 | −20.874 | 15.911 | 1.00 | 26.16 | C |
| ATOM | 2251 | C | LEU | L | 73 | 56.767 | −17.802 | 17.948 | 1.00 | 19.08 | C |
| ATOM | 2252 | O | LEU | L | 73 | 56.100 | −17.186 | 18.783 | 1.00 | 17.99 | O |
| ATOM | 2253 | N | LYS | L | 74 | 57.320 | −17.215 | 16.896 | 1.00 | 19.39 | N |
| ATOM | 2254 | CA | LYS | L | 74 | 57.154 | −15.796 | 16.668 | 1.00 | 19.85 | C |
| ATOM | 2255 | CB | LYS | L | 74 | 58.472 | −15.088 | 16.916 | 1.00 | 20.73 | C |
| ATOM | 2256 | CG | LYS | L | 74 | 58.710 | −14.713 | 18.370 | 1.00 | 25.03 | C |
| ATOM | 2257 | CD | LYS | L | 74 | 59.786 | −13.643 | 18.453 | 1.00 | 30.30 | C |
| ATOM | 2258 | CE | LYS | L | 74 | 61.136 | −14.277 | 18.662 | 1.00 | 33.67 | C |
| ATOM | 2259 | NZ | LYS | L | 74 | 61.231 | −14.754 | 20.070 | 1.00 | 35.57 | N |
| ATOM | 2260 | C | LYS | L | 74 | 56.700 | −15.462 | 15.250 | 1.00 | 19.22 | C |
| ATOM | 2261 | O | LYS | L | 74 | 57.102 | −16.116 | 14.289 | 1.00 | 18.04 | O |
| ATOM | 2262 | N | ILE | L | 75 | 55.917 | −14.391 | 15.151 | 1.00 | 18.65 | N |
| ATOM | 2263 | CA | ILE | L | 75 | 55.475 | −13.849 | 13.872 | 1.00 | 18.93 | C |
| ATOM | 2264 | CB | ILE | L | 75 | 53.939 | −13.876 | 13.746 | 1.00 | 19.04 | C |
| ATOM | 2265 | CG1 | ILE | L | 75 | 53.409 | −15.299 | 14.010 | 1.00 | 19.92 | C |
| ATOM | 2266 | CD1 | ILE | L | 75 | 51.882 | −15.435 | 14.209 | 1.00 | 20.40 | C |
| ATOM | 2267 | CG2 | ILE | L | 75 | 53.505 | −13.308 | 12.351 | 1.00 | 18.69 | C |
| ATOM | 2268 | C | ILE | L | 75 | 55.984 | −12.419 | 13.789 | 1.00 | 18.72 | C |
| ATOM | 2269 | O | ILE | L | 75 | 55.657 | −11.600 | 14.651 | 1.00 | 18.32 | O |
| ATOM | 2270 | N | SER | L | 76 | 56.799 | −12.121 | 12.778 | 1.00 | 18.28 | N |
| ATOM | 2271 | CA | SER | L | 76 | 57.436 | −10.789 | 12.673 | 1.00 | 18.64 | C |
| ATOM | 2272 | CB | SER | L | 76 | 58.400 | −10.731 | 11.473 | 1.00 | 18.98 | C |
| ATOM | 2273 | OG | SER | L | 76 | 57.737 | −11.180 | 10.298 | 1.00 | 18.55 | O |
| ATOM | 2274 | C | SER | L | 76 | 56.401 | −9.677 | 12.559 | 1.00 | 19.29 | C |
| ATOM | 2275 | O | SER | L | 76 | 56.459 | −8.675 | 13.295 | 1.00 | 19.11 | O |
| ATOM | 2276 | N | ARG | L | 77 | 55.448 | −9.856 | 11.639 | 1.00 | 19.61 | N |
| ATOM | 2277 | CA | ARG | L | 77 | 54.335 | −8.934 | 11.470 | 1.00 | 19.93 | C |
| ATOM | 2278 | CB | ARG | L | 77 | 54.585 | −7.944 | 10.332 | 1.00 | 20.00 | C |
| ATOM | 2279 | CG | ARG | L | 77 | 55.689 | −6.907 | 10.598 | 1.00 | 21.10 | C |
| ATOM | 2280 | CD | ARG | L | 77 | 55.762 | −5.887 | 9.485 | 1.00 | 21.67 | C |
| ATOM | 2281 | NE | ARG | L | 77 | 54.637 | −4.953 | 9.542 | 1.00 | 22.94 | N |
| ATOM | 2282 | CZ | ARG | L | 77 | 54.159 | −4.289 | 8.496 | 1.00 | 24.00 | C |
| ATOM | 2283 | NH1 | ARG | L | 77 | 54.713 | −4.445 | 7.296 | 1.00 | 24.89 | N |
| ATOM | 2284 | NH2 | ARG | L | 77 | 53.148 | −3.449 | 8.648 | 1.00 | 22.24 | N |
| ATOM | 2285 | C | ARG | L | 77 | 53.103 | −9.771 | 11.164 | 1.00 | 20.73 | C |
| ATOM | 2286 | O | ARG | L | 77 | 53.078 | −10.515 | 10.177 | 1.00 | 20.97 | O |
| ATOM | 2287 | N | VAL | L | 78 | 52.102 | −9.675 | 12.023 | 1.00 | 20.46 | N |
| ATOM | 2288 | CA | VAL | L | 78 | 50.907 | −10.496 | 11.894 | 1.00 | 21.10 | C |
| ATOM | 2289 | CB | VAL | L | 78 | 50.035 | −10.410 | 13.143 | 1.00 | 20.72 | C |
| ATOM | 2290 | CG1 | VAL | L | 78 | 48.620 | −11.000 | 12.888 | 1.00 | 22.44 | C |
| ATOM | 2291 | CG2 | VAL | L | 78 | 50.698 | −11.167 | 14.290 | 1.00 | 22.48 | C |
| ATOM | 2292 | C | VAL | L | 78 | 50.082 | −10.113 | 10.673 | 1.00 | 21.76 | C |
| ATOM | 2293 | O | VAL | L | 78 | 49.981 | −8.928 | 10.302 | 1.00 | 20.97 | O |
| ATOM | 2294 | N | GLU | L | 79 | 49.482 | −11.127 | 10.057 | 1.00 | 22.21 | N |
| ATOM | 2295 | CA | GLU | L | 79 | 48.510 | −10.894 | 8.981 | 1.00 | 22.92 | C |
| ATOM | 2296 | CB | GLU | L | 79 | 49.077 | −11.419 | 7.663 | 1.00 | 23.26 | C |
| ATOM | 2297 | CG | GLU | L | 79 | 50.473 | −10.862 | 7.405 | 1.00 | 27.01 | C |
| ATOM | 2298 | CD | GLU | L | 79 | 50.931 | −11.032 | 5.992 | 1.00 | 29.63 | C |
| ATOM | 2299 | OE1 | GLU | L | 79 | 50.283 | −11.781 | 5.231 | 1.00 | 31.91 | O |
| ATOM | 2300 | OE2 | GLU | L | 79 | 51.944 | −10.401 | 5.643 | 1.00 | 32.09 | O |
| ATOM | 2301 | C | GLU | L | 79 | 47.195 | −11.569 | 9.337 | 1.00 | 22.41 | C |
| ATOM | 2302 | O | GLU | L | 79 | 47.164 | −12.480 | 10.174 | 1.00 | 21.92 | O |
| ATOM | 2303 | N | ALA | L | 80 | 46.115 | −11.120 | 8.703 | 1.00 | 22.55 | N |
| ATOM | 2304 | CA | ALA | L | 80 | 44.772 | −11.630 | 8.981 | 1.00 | 23.11 | C |
| ATOM | 2305 | CB | ALA | L | 80 | 43.758 | −10.970 | 8.042 | 1.00 | 23.43 | C |
| ATOM | 2306 | C | ALA | L | 80 | 44.681 | −13.167 | 8.917 | 1.00 | 23.45 | C |
| ATOM | 2307 | O | ALA | L | 80 | 44.010 | −13.802 | 9.746 | 1.00 | 22.84 | O |
| ATOM | 2308 | N | GLU | L | 81 | 45.414 | −13.754 | 7.969 | 1.00 | 23.85 | N |
| ATOM | 2309 | CA | GLU | L | 81 | 45.436 | −15.203 | 7.737 | 1.00 | 24.84 | C |
| ATOM | 2310 | CB | GLU | L | 81 | 46.192 | −15.506 | 6.424 | 1.00 | 25.79 | C |
| ATOM | 2311 | CG | GLU | L | 81 | 45.542 | −14.866 | 5.201 | 1.00 | 29.88 | C |
| ATOM | 2312 | CD | GLU | L | 81 | 45.733 | −13.339 | 5.127 | 1.00 | 33.98 | C |
| ATOM | 2313 | OE1 | GLU | L | 81 | 46.674 | −12.802 | 5.759 | 1.00 | 35.75 | O |
| ATOM | 2314 | OE2 | GLU | L | 81 | 44.927 | −12.682 | 4.437 | 1.00 | 36.56 | O |
| ATOM | 2315 | C | GLU | L | 81 | 46.069 | −16.000 | 8.869 | 1.00 | 24.28 | C |
| ATOM | 2316 | O | GLU | L | 81 | 45.981 | −17.227 | 8.885 | 1.00 | 24.68 | O |
| ATOM | 2317 | N | ASP | L | 82 | 46.720 | −15.317 | 9.809 | 1.00 | 23.03 | N |
| ATOM | 2318 | CA | ASP | L | 82 | 47.471 | −16.014 | 10.869 | 1.00 | 22.19 | C |
| ATOM | 2319 | CB | ASP | L | 82 | 48.624 | −15.139 | 11.377 | 1.00 | 21.87 | C |
| ATOM | 2320 | CG | ASP | L | 82 | 49.643 | −14.812 | 10.287 | 1.00 | 22.34 | C |
| ATOM | 2321 | OD1 | ASP | L | 82 | 49.763 | −15.582 | 9.315 | 1.00 | 22.39 | O |
| ATOM | 2322 | OD2 | ASP | L | 82 | 50.332 | −13.770 | 10.390 | 1.00 | 22.50 | O |
| ATOM | 2323 | C | ASP | L | 82 | 46.593 | −16.464 | 12.038 | 1.00 | 22.26 | C |
| ATOM | 2324 | O | ASP | L | 82 | 47.070 | −17.116 | 12.970 | 1.00 | 22.13 | O |
| ATOM | 2325 | N | VAL | L | 83 | 45.305 | −16.122 | 11.971 | 1.00 | 22.13 | N |
| ATOM | 2326 | CA | VAL | L | 83 | 44.323 | −16.434 | 13.020 | 1.00 | 22.19 | C |
| ATOM | 2327 | CB | VAL | L | 83 | 43.006 | −15.680 | 12.723 | 1.00 | 22.62 | C |
| ATOM | 2328 | CG1 | VAL | L | 83 | 41.850 | −16.224 | 13.522 | 1.00 | 25.36 | C |
| ATOM | 2329 | CG2 | VAL | L | 83 | 43.204 | −14.201 | 13.028 | 1.00 | 21.65 | C |
| ATOM | 2330 | C | VAL | L | 83 | 44.075 | −17.944 | 13.172 | 1.00 | 21.99 | C |
| ATOM | 2331 | O | VAL | L | 83 | 44.147 | −18.674 | 12.193 | 1.00 | 21.54 | O |
| ATOM | 2332 | N | GLY | L | 84 | 43.800 | −18.385 | 14.406 | 1.00 | 21.36 | N |
| ATOM | 2333 | CA | GLY | L | 84 | 43.416 | −19.773 | 14.685 | 1.00 | 20.99 | C |
| ATOM | 2334 | C | GLY | L | 84 | 44.127 | −20.289 | 15.914 | 1.00 | 20.60 | C |
| ATOM | 2335 | O | GLY | L | 84 | 44.668 | −19.499 | 16.692 | 1.00 | 20.15 | O |
| ATOM | 2336 | N | VAL | L | 85 | 44.145 | −21.614 | 16.091 | 1.00 | 20.19 | N |
| ATOM | 2337 | CA | VAL | L | 85 | 44.728 | −22.209 | 17.297 | 1.00 | 19.88 | C |
| ATOM | 2338 | CB | VAL | L | 85 | 43.775 | −23.259 | 17.951 | 1.00 | 20.62 | C |
| ATOM | 2339 | CG1 | VAL | L | 85 | 44.415 | −23.889 | 19.179 | 1.00 | 20.36 | C |
| ATOM | 2340 | CG2 | VAL | L | 85 | 42.420 | −22.604 | 18.318 | 1.00 | 20.61 | C |
| ATOM | 2341 | C | VAL | L | 85 | 46.100 | −22.820 | 17.002 | 1.00 | 20.28 | C |
| ATOM | 2342 | O | VAL | L | 85 | 46.234 | −23.689 | 16.135 | 1.00 | 19.75 | O |
| ATOM | 2343 | N | TYR | L | 86 | 47.111 | −22.335 | 17.721 | 1.00 | 19.32 | N |
| ATOM | 2344 | CA | TYR | L | 86 | 48.481 | −22.810 | 17.583 | 1.00 | 19.65 | C |
| ATOM | 2345 | CB | TYR | L | 86 | 49.479 | −21.663 | 17.821 | 1.00 | 19.03 | C |
| ATOM | 2346 | CG | TYR | L | 86 | 49.474 | −20.693 | 16.680 | 1.00 | 19.92 | C |
| ATOM | 2347 | CD1 | TYR | L | 86 | 48.487 | −19.691 | 16.581 | 1.00 | 16.75 | C |
| ATOM | 2348 | CE1 | TYR | L | 86 | 48.462 | −18.815 | 15.500 | 1.00 | 20.09 | C |
| ATOM | 2349 | CZ | TYR | L | 86 | 49.445 | −18.944 | 14.504 | 1.00 | 19.73 | C |
| ATOM | 2350 | OH | TYR | L | 86 | 49.471 | −18.109 | 13.418 | 1.00 | 18.58 | O |
| ATOM | 2351 | CE2 | TYR | L | 86 | 50.414 | −19.931 | 14.593 | 1.00 | 20.21 | C |
| ATOM | 2352 | CD2 | TYR | L | 86 | 50.421 | −20.800 | 15.669 | 1.00 | 18.33 | C |
| ATOM | 2353 | C | TYR | L | 86 | 48.711 | −23.906 | 18.602 | 1.00 | 19.59 | C |
| ATOM | 2354 | O | TYR | L | 86 | 48.406 | −23.723 | 19.785 | 1.00 | 19.53 | O |
| ATOM | 2355 | N | PHE | L | 87 | 49.254 | −25.036 | 18.138 | 1.00 | 19.44 | N |
| ATOM | 2356 | CA | PHE | L | 87 | 49.632 | −26.130 | 19.031 | 1.00 | 19.38 | C |
| ATOM | 2357 | CB | PHE | L | 87 | 48.880 | −27.420 | 18.679 | 1.00 | 19.68 | C |
| ATOM | 2358 | CG | PHE | L | 87 | 47.396 | −27.389 | 18.953 | 1.00 | 19.97 | C |
| ATOM | 2359 | CD1 | PHE | L | 87 | 46.903 | −27.655 | 20.224 | 1.00 | 23.27 | C |
| ATOM | 2360 | CE1 | PHE | L | 87 | 45.535 | −27.656 | 20.488 | 1.00 | 23.51 | C |
| ATOM | 2361 | CZ | PHE | L | 87 | 44.631 | −27.427 | 19.435 | 1.00 | 25.17 | C |
| ATOM | 2362 | CE2 | PHE | L | 87 | 45.118 | −27.175 | 18.150 | 1.00 | 23.68 | C |
| ATOM | 2363 | CD2 | PHE | L | 87 | 46.494 | −27.169 | 17.920 | 1.00 | 22.93 | C |
| ATOM | 2364 | C | PHE | L | 87 | 51.120 | −26.418 | 18.911 | 1.00 | 19.41 | C |
| ATOM | 2365 | O | PHE | L | 87 | 51.667 | −26.461 | 17.792 | 1.00 | 19.64 | O |
| ATOM | 2366 | N | CYS | L | 88 | 51.784 | −26.585 | 20.053 | 1.00 | 19.10 | N |
| ATOM | 2367 | CA | CYS | L | 88 | 53.117 | −27.167 | 20.060 | 1.00 | 20.14 | C |
| ATOM | 2368 | CB | CYS | L | 88 | 54.018 | −26.602 | 21.183 | 1.00 | 20.40 | C |
| ATOM | 2369 | SG | CYS | L | 88 | 53.472 | −26.757 | 22.891 | 1.00 | 23.03 | S |
| ATOM | 2370 | C | CYS | L | 88 | 52.965 | −28.678 | 20.143 | 1.00 | 19.65 | C |
| ATOM | 2371 | O | CYS | L | 88 | 51.958 | −29.190 | 20.654 | 1.00 | 19.74 | O |
| ATOM | 2372 | N | SER | L | 89 | 53.955 | −29.382 | 19.610 | 1.00 | 19.99 | N |
| ATOM | 2373 | CA | SER | L | 89 | 53.896 | −30.843 | 19.421 | 1.00 | 19.13 | C |
| ATOM | 2374 | CB | SER | L | 89 | 53.554 | −31.160 | 17.965 | 1.00 | 19.43 | C |
| ATOM | 2375 | OG | SER | L | 89 | 53.696 | −32.559 | 17.693 | 1.00 | 20.81 | O |
| ATOM | 2376 | C | SER | L | 89 | 55.269 | −31.394 | 19.692 | 1.00 | 18.38 | C |
| ATOM | 2377 | O | SER | L | 89 | 56.239 | −30.854 | 19.169 | 1.00 | 17.95 | O |
| ATOM | 2378 | N | GLN | L | 90 | 55.377 | −32.466 | 20.483 | 1.00 | 18.15 | N |
| ATOM | 2379 | CA | GLN | L | 90 | 56.684 | −33.144 | 20.610 | 1.00 | 17.13 | C |
| ATOM | 2380 | CB | GLN | L | 90 | 57.242 | −33.059 | 22.046 | 1.00 | 17.79 | C |
| ATOM | 2381 | CG | GLN | L | 90 | 56.516 | −33.913 | 23.130 | 1.00 | 16.22 | C |
| ATOM | 2382 | CD | GLN | L | 90 | 57.009 | −35.365 | 23.201 | 1.00 | 16.94 | C |
| ATOM | 2383 | OE1 | GLN | L | 90 | 58.116 | −35.676 | 22.750 | 1.00 | 16.76 | O |
| ATOM | 2384 | NE2 | GLN | L | 90 | 56.178 | −36.262 | 23.769 | 1.00 | 14.32 | N |
| ATOM | 2385 | C | GLN | L | 90 | 56.673 | −34.592 | 20.089 | 1.00 | 17.18 | C |
| ATOM | 2386 | O | GLN | L | 90 | 55.715 | −35.341 | 20.309 | 1.00 | 16.84 | O |
| ATOM | 2387 | N | SER | L | 91 | 57.741 | −34.980 | 19.393 | 1.00 | 16.69 | N |
| ATOM | 2388 | CA | SER | L | 91 | 57.889 | −36.356 | 18.940 | 1.00 | 16.62 | C |
| ATOM | 2389 | CB | SER | L | 91 | 57.686 | −36.452 | 17.417 | 1.00 | 16.85 | C |
| ATOM | 2390 | OG | SER | L | 91 | 58.553 | −35.563 | 16.739 | 1.00 | 18.12 | O |
| ATOM | 2391 | C | SER | L | 91 | 59.248 | −36.914 | 19.334 | 1.00 | 16.71 | C |
| ATOM | 2392 | O | SER | L | 91 | 59.792 | −37.778 | 18.648 | 1.00 | 16.97 | O |
| ATOM | 2393 | N | THR | L | 92 | 59.782 | −36.420 | 20.452 | 1.00 | 16.64 | N |
| ATOM | 2394 | CA | THR | L | 92 | 61.018 | −36.946 | 21.024 | 1.00 | 16.51 | C |
| ATOM | 2395 | CB | THR | L | 92 | 61.736 | −35.887 | 21.897 | 1.00 | 16.38 | C |
| ATOM | 2396 | OG1 | THR | L | 92 | 62.148 | −34.792 | 21.077 | 1.00 | 16.82 | O |
| ATOM | 2397 | CG2 | THR | L | 92 | 62.957 | −36.483 | 22.570 | 1.00 | 16.51 | C |
| ATOM | 2398 | C | THR | L | 92 | 60.770 | −38.168 | 21.884 | 1.00 | 16.65 | C |
| ATOM | 2399 | O | THR | L | 92 | 61.554 | −39.122 | 21.850 | 1.00 | 16.08 | O |
| ATOM | 2400 | N | HIS | L | 93 | 59.701 | −38.121 | 22.677 | 1.00 | 16.64 | N |
| ATOM | 2401 | CA | HIS | L | 93 | 59.380 | −39.175 | 23.636 | 1.00 | 17.31 | C |
| ATOM | 2402 | CB | HIS | L | 93 | 59.398 | −38.623 | 25.087 | 1.00 | 17.68 | C |
| ATOM | 2403 | CG | HIS | L | 93 | 60.682 | −37.950 | 25.482 | 1.00 | 19.46 | C |
| ATOM | 2404 | ND1 | HIS | L | 93 | 61.795 | −38.646 | 25.903 | 1.00 | 22.73 | N |
| ATOM | 2405 | CE1 | HIS | L | 93 | 62.772 | −37.794 | 26.174 | 1.00 | 23.30 | C |
| ATOM | 2406 | NE2 | HIS | L | 93 | 62.322 | −36.567 | 25.967 | 1.00 | 23.84 | N |
| ATOM | 2407 | CD2 | HIS | L | 93 | 61.017 | −36.637 | 25.545 | 1.00 | 22.08 | C |
| ATOM | 2408 | C | HIS | L | 93 | 57.978 | −39.736 | 23.387 | 1.00 | 16.96 | C |
| ATOM | 2409 | O | HIS | L | 93 | 57.024 | −38.972 | 23.287 | 1.00 | 17.10 | O |
| ATOM | 2410 | N | PHE | L | 94 | 57.837 | −41.054 | 23.324 | 1.00 | 16.94 | N |
| ATOM | 2411 | CA | PHE | L | 94 | 56.509 | −41.687 | 23.405 | 1.00 | 17.43 | C |
| ATOM | 2412 | CB | PHE | L | 94 | 56.568 | −43.159 | 22.979 | 1.00 | 17.61 | C |
| ATOM | 2413 | CG | PHE | L | 94 | 56.730 | −43.364 | 21.501 | 1.00 | 19.41 | C |
| ATOM | 2414 | CD1 | PHE | L | 94 | 55.703 | −43.047 | 20.623 | 1.00 | 19.88 | C |
| ATOM | 2415 | CE1 | PHE | L | 94 | 55.847 | −43.246 | 19.254 | 1.00 | 21.80 | C |
| ATOM | 2416 | CZ | PHE | L | 94 | 57.024 | −43.782 | 18.755 | 1.00 | 21.99 | C |
| ATOM | 2417 | CE2 | PHE | L | 94 | 58.048 | −44.118 | 19.616 | 1.00 | 23.66 | C |
| ATOM | 2418 | CD2 | PHE | L | 94 | 57.904 | −43.905 | 20.995 | 1.00 | 21.77 | C |
| ATOM | 2419 | C | PHE | L | 94 | 56.003 | −41.649 | 24.849 | 1.00 | 17.42 | C |
| ATOM | 2420 | O | PHE | L | 94 | 56.790 | −41.790 | 25.772 | 1.00 | 17.34 | O |
| ATOM | 2421 | N | PRO | L | 95 | 54.691 | −41.450 | 25.054 | 1.00 | 17.95 | N |
| ATOM | 2422 | CA | PRO | L | 95 | 53.678 | −41.139 | 24.027 | 1.00 | 17.88 | C |
| ATOM | 2423 | CB | PRO | L | 95 | 52.353 | −41.260 | 24.786 | 1.00 | 18.25 | C |
| ATOM | 2424 | CG | PRO | L | 95 | 52.728 | −40.937 | 26.236 | 1.00 | 18.23 | C |
| ATOM | 2425 | CD | PRO | L | 95 | 54.108 | −41.541 | 26.412 | 1.00 | 18.06 | C |
| ATOM | 2426 | C | PRO | L | 95 | 53.856 | −39.718 | 23.516 | 1.00 | 18.01 | C |
| ATOM | 2427 | O | PRO | L | 95 | 54.185 | −38.812 | 24.295 | 1.00 | 18.30 | O |
| ATOM | 2428 | N | PHE | L | 96 | 53.684 | −39.521 | 22.212 | 1.00 | 17.52 | N |
| ATOM | 2429 | CA | PHE | L | 96 | 53.796 | −38.186 | 21.649 | 1.00 | 17.03 | C |
| ATOM | 2430 | CB | PHE | L | 96 | 53.670 | −38.196 | 20.116 | 1.00 | 17.16 | C |
| ATOM | 2431 | CG | PHE | L | 96 | 54.824 | −38.895 | 19.408 | 1.00 | 17.38 | C |
| ATOM | 2432 | CD1 | PHE | L | 96 | 54.745 | −39.184 | 18.047 | 1.00 | 18.46 | C |
| ATOM | 2433 | CE1 | PHE | L | 96 | 55.805 | −39.820 | 17.390 | 1.00 | 17.22 | C |
| ATOM | 2434 | CZ | PHE | L | 96 | 56.954 | −40.186 | 18.099 | 1.00 | 17.30 | C |
| ATOM | 2435 | CE2 | PHE | L | 96 | 57.045 | −39.927 | 19.460 | 1.00 | 18.13 | C |
| ATOM | 2436 | CD2 | PHE | L | 96 | 55.978 | −39.275 | 20.109 | 1.00 | 17.18 | C |
| ATOM | 2437 | C | PHE | L | 96 | 52.714 | −37.339 | 22.286 | 1.00 | 16.90 | C |
| ATOM | 2438 | O | PHE | L | 96 | 51.638 | −37.847 | 22.604 | 1.00 | 16.76 | O |
| ATOM | 2439 | N | THR | L | 97 | 53.007 | −36.060 | 22.499 | 1.00 | 16.89 | N |
| ATOM | 2440 | CA | THR | L | 97 | 52.059 | −35.179 | 23.210 | 1.00 | 17.70 | C |
| ATOM | 2441 | CB | THR | L | 97 | 52.463 | −34.971 | 24.706 | 1.00 | 17.37 | C |
| ATOM | 2442 | OG1 | THR | L | 97 | 53.819 | −34.508 | 24.790 | 1.00 | 18.61 | O |
| ATOM | 2443 | CG2 | THR | L | 97 | 52.327 | −36.270 | 25.491 | 1.00 | 17.86 | C |
| ATOM | 2444 | C | THR | L | 97 | 51.920 | −33.832 | 22.535 | 1.00 | 17.93 | C |
| ATOM | 2445 | O | THR | L | 97 | 52.728 | −33.479 | 21.676 | 1.00 | 17.67 | O |
| ATOM | 2446 | N | PHE | L | 98 | 50.893 | −33.080 | 22.946 | 1.00 | 17.93 | N |
| ATOM | 2447 | CA | PHE | L | 98 | 50.612 | −31.757 | 22.419 | 1.00 | 19.14 | C |
| ATOM | 2448 | CB | PHE | L | 98 | 49.372 | −31.803 | 21.520 | 1.00 | 18.77 | C |
| ATOM | 2449 | CG | PHE | L | 98 | 49.552 | −32.610 | 20.278 | 1.00 | 19.64 | C |
| ATOM | 2450 | CD1 | PHE | L | 98 | 49.182 | −33.951 | 20.243 | 1.00 | 18.49 | C |
| ATOM | 2451 | CE1 | PHE | L | 98 | 49.342 | −34.694 | 19.074 | 1.00 | 21.10 | C |
| ATOM | 2452 | CZ | PHE | L | 98 | 49.866 | −34.088 | 17.939 | 1.00 | 20.05 | C |
| ATOM | 2453 | CE2 | PHE | L | 98 | 50.219 | −32.760 | 17.965 | 1.00 | 20.31 | C |
| ATOM | 2454 | CD2 | PHE | L | 98 | 50.058 | −32.021 | 19.131 | 1.00 | 17.71 | C |
| ATOM | 2455 | C | PHE | L | 98 | 50.312 | −30.779 | 23.551 | 1.00 | 18.98 | C |
| ATOM | 2456 | O | PHE | L | 98 | 49.804 | −31.177 | 24.594 | 1.00 | 18.94 | O |
| ATOM | 2457 | N | GLY | L | 99 | 50.650 | −29.510 | 23.336 | 1.00 | 19.48 | N |
| ATOM | 2458 | CA | GLY | L | 99 | 50.179 | −28.429 | 24.213 | 1.00 | 19.60 | C |
| ATOM | 2459 | C | GLY | L | 99 | 48.675 | −28.234 | 24.052 | 1.00 | 19.97 | C |
| ATOM | 2460 | O | GLY | L | 99 | 48.064 | −28.756 | 23.103 | 1.00 | 18.55 | O |
| ATOM | 2461 | N | GLN | L | 100 | 48.075 | −27.481 | 24.973 | 1.00 | 20.74 | N |
| ATOM | 2462 | CA | GLN | L | 100 | 46.631 | −27.299 | 24.949 | 1.00 | 22.30 | C |
| ATOM | 2463 | CB | GLN | L | 100 | 46.049 | −27.123 | 26.363 | 1.00 | 23.60 | C |
| ATOM | 2464 | CG | GLN | L | 100 | 46.016 | −28.429 | 27.207 | 1.00 | 26.65 | C |
| ATOM | 2465 | CD | GLN | L | 100 | 45.782 | −29.709 | 26.377 | 1.00 | 29.21 | C |
| ATOM | 2466 | OE1 | GLN | L | 100 | 46.697 | −30.526 | 26.200 | 1.00 | 31.84 | O |
| ATOM | 2467 | NE2 | GLN | L | 100 | 44.570 | −29.872 | 25.863 | 1.00 | 26.34 | N |
| ATOM | 2468 | C | GLN | L | 100 | 46.140 | −26.211 | 23.982 | 1.00 | 22.02 | C |
| ATOM | 2469 | O | GLN | L | 100 | 44.941 | −26.038 | 23.815 | 1.00 | 21.52 | O |
| ATOM | 2470 | N | GLY | L | 101 | 47.059 | −25.509 | 23.321 | 1.00 | 21.37 | N |
| ATOM | 2471 | CA | GLY | L | 101 | 46.656 | −24.571 | 22.282 | 1.00 | 21.51 | C |
| ATOM | 2472 | C | GLY | L | 101 | 46.677 | −23.119 | 22.721 | 1.00 | 21.97 | C |
| ATOM | 2473 | O | GLY | L | 101 | 46.379 | −22.801 | 23.881 | 1.00 | 21.93 | O |
| ATOM | 2474 | N | THR | L | 102 | 47.050 | −22.241 | 21.796 | 1.00 | 22.20 | N |
| ATOM | 2475 | CA | THR | L | 102 | 46.970 | −20.783 | 22.018 | 1.00 | 22.11 | C |
| ATOM | 2476 | CB | THR | L | 102 | 48.366 | −20.129 | 21.989 | 1.00 | 22.09 | C |
| ATOM | 2477 | OG1 | THR | L | 102 | 49.100 | −20.494 | 23.166 | 1.00 | 21.45 | O |
| ATOM | 2478 | CG2 | THR | L | 102 | 48.248 | −18.548 | 21.915 | 1.00 | 21.35 | C |
| ATOM | 2479 | C | THR | L | 102 | 46.135 | −20.201 | 20.898 | 1.00 | 22.80 | C |
| ATOM | 2480 | O | THR | L | 102 | 46.453 | −20.421 | 19.736 | 1.00 | 23.20 | O |
| ATOM | 2481 | N | LYS | L | 103 | 45.074 | −19.463 | 21.223 | 1.00 | 23.46 | N |
| ATOM | 2482 | CA | LYS | L | 103 | 44.181 | −18.950 | 20.177 | 1.00 | 23.81 | C |
| ATOM | 2483 | CB | LYS | L | 103 | 42.696 | −19.061 | 20.578 | 1.00 | 24.73 | C |
| ATOM | 2484 | CG | LYS | L | 103 | 41.697 | −18.749 | 19.429 | 1.00 | 26.66 | C |
| ATOM | 2485 | CD | LYS | L | 103 | 40.221 | −18.941 | 19.863 | 1.00 | 32.57 | C |
| ATOM | 2486 | CE | LYS | L | 103 | 39.248 | −18.442 | 18.770 | 1.00 | 36.78 | C |
| ATOM | 2487 | NZ | LYS | L | 103 | 37.811 | −18.286 | 19.239 | 1.00 | 39.94 | N |
| ATOM | 2488 | C | LYS | L | 103 | 44.530 | −17.516 | 19.807 | 1.00 | 23.64 | C |
| ATOM | 2489 | O | LYS | L | 103 | 44.436 | −16.591 | 20.637 | 1.00 | 22.88 | O |
| ATOM | 2490 | N | LEU | L | 104 | 44.919 | −17.342 | 18.551 | 1.00 | 22.92 | N |
| ATOM | 2491 | CA | LEU | L | 104 | 45.211 | −16.027 | 18.009 | 1.00 | 22.43 | C |
| ATOM | 2492 | CB | LEU | L | 104 | 46.395 | −16.151 | 17.032 | 1.00 | 22.96 | C |
| ATOM | 2493 | CG | LEU | L | 104 | 47.189 | −14.898 | 16.717 | 1.00 | 25.15 | C |
| ATOM | 2494 | CD1 | LEU | L | 104 | 48.667 | −15.165 | 16.437 | 1.00 | 25.59 | C |
| ATOM | 2495 | CD2 | LEU | L | 104 | 46.552 | −14.198 | 15.507 | 1.00 | 28.28 | C |
| ATOM | 2496 | C | LEU | L | 104 | 43.933 | −15.491 | 17.336 | 1.00 | 22.25 | C |
| ATOM | 2497 | O | LEU | L | 104 | 43.340 | −16.179 | 16.519 | 1.00 | 21.30 | O |
| ATOM | 2498 | N | GLU | L | 105 | 43.497 | −14.292 | 17.726 | 1.00 | 21.57 | N |
| ATOM | 2499 | CA | GLU | L | 105 | 42.285 | −13.662 | 17.182 | 1.00 | 22.26 | C |
| ATOM | 2500 | CB | GLU | L | 105 | 41.109 | −13.709 | 18.192 | 1.00 | 22.40 | C |
| ATOM | 2501 | CG | GLU | L | 105 | 41.372 | −12.972 | 19.541 | 1.00 | 27.32 | C |
| ATOM | 2502 | CD | GLU | L | 105 | 40.144 | −12.202 | 20.116 | 1.00 | 33.59 | C |
| ATOM | 2503 | OE1 | GLU | L | 105 | 40.285 | −11.527 | 21.159 | 1.00 | 36.14 | O |
| ATOM | 2504 | OE2 | GLU | L | 105 | 39.039 | −12.248 | 19.538 | 1.00 | 36.44 | O |
| ATOM | 2505 | C | GLU | L | 105 | 42.614 | −12.215 | 16.847 | 1.00 | 21.02 | C |
| ATOM | 2506 | O | GLU | L | 105 | 43.613 | −11.681 | 17.332 | 1.00 | 20.82 | O |
| ATOM | 2507 | N | ILE | L | 106 | 41.766 | −11.576 | 16.044 | 1.00 | 20.40 | N |
| ATOM | 2508 | CA | ILE | L | 106 | 42.012 | −10.200 | 15.614 | 1.00 | 19.76 | C |
| ATOM | 2509 | CB | ILE | L | 106 | 41.256 | −9.851 | 14.289 | 1.00 | 19.46 | C |
| ATOM | 2510 | CG1 | ILE | L | 106 | 41.787 | −10.676 | 13.094 | 1.00 | 19.99 | C |
| ATOM | 2511 | CD1 | ILE | L | 106 | 43.328 | −10.596 | 12.864 | 1.00 | 20.73 | C |
| ATOM | 2512 | CG2 | ILE | L | 106 | 41.302 | −8.324 | 14.006 | 1.00 | 19.20 | C |
| ATOM | 2513 | C | ILE | L | 106 | 41.585 | −9.241 | 16.719 | 1.00 | 19.80 | C |
| ATOM | 2514 | O | ILE | L | 106 | 40.467 | −9.335 | 17.230 | 1.00 | 19.71 | O |
| ATOM | 2515 | N | LYS | L | 107 | 42.485 | −8.343 | 17.109 | 1.00 | 19.66 | N |
| ATOM | 2516 | CA | LYS | L | 107 | 42.116 | −7.281 | 18.039 | 1.00 | 20.14 | C |
| ATOM | 2517 | CB | LYS | L | 107 | 43.334 | −6.803 | 18.836 | 1.00 | 20.36 | C |
| ATOM | 2518 | CG | LYS | L | 107 | 42.936 | −5.855 | 19.988 | 1.00 | 22.07 | C |
| ATOM | 2519 | CD | LYS | L | 107 | 44.114 | −5.017 | 20.472 | 1.00 | 25.05 | C |
| ATOM | 2520 | CE | LYS | L | 107 | 43.654 | −3.979 | 21.494 | 1.00 | 27.02 | C |
| ATOM | 2521 | NZ | LYS | L | 107 | 43.430 | −4.649 | 22.799 | 1.00 | 28.71 | N |
| ATOM | 2522 | C | LYS | L | 107 | 41.515 | −6.118 | 17.252 | 1.00 | 19.36 | C |
| ATOM | 2523 | O | LYS | L | 107 | 42.114 | −5.655 | 16.287 | 1.00 | 19.02 | O |
| ATOM | 2524 | N | ARG | L | 108 | 40.329 | −5.672 | 17.650 | 1.00 | 19.41 | N |
| ATOM | 2525 | CA | ARG | L | 108 | 39.710 | −4.488 | 17.055 | 1.00 | 19.66 | C |
| ATOM | 2526 | CB | ARG | L | 108 | 38.567 | −4.879 | 16.117 | 1.00 | 19.36 | C |
| ATOM | 2527 | CG | ARG | L | 108 | 37.430 | −5.635 | 16.778 | 1.00 | 19.52 | C |
| ATOM | 2528 | CD | ARG | L | 108 | 36.137 | −5.440 | 16.018 | 1.00 | 19.99 | C |
| ATOM | 2529 | NE | ARG | L | 108 | 35.554 | −4.118 | 16.270 | 1.00 | 17.84 | N |
| ATOM | 2530 | CZ | ARG | L | 108 | 34.599 | −3.567 | 15.535 | 1.00 | 18.46 | C |
| ATOM | 2531 | NH1 | ARG | L | 108 | 34.134 | −2.361 | 15.848 | 1.00 | 17.43 | N |
| ATOM | 2532 | NH2 | ARG | L | 108 | 34.107 | −4.213 | 14.486 | 1.00 | 20.37 | N |
| ATOM | 2533 | C | ARG | L | 108 | 39.218 | −3.569 | 18.171 | 1.00 | 19.77 | C |
| ATOM | 2534 | O | ARG | L | 108 | 39.334 | −3.923 | 19.343 | 1.00 | 20.41 | O |
| ATOM | 2535 | N | THR | L | 109 | 38.698 | −2.392 | 17.821 | 1.00 | 20.03 | N |
| ATOM | 2536 | CA | THR | L | 109 | 38.109 | −1.483 | 18.817 | 1.00 | 19.98 | C |
| ATOM | 2537 | CB | THR | L | 109 | 37.578 | −0.187 | 18.198 | 1.00 | 20.31 | C |
| ATOM | 2538 | OG1 | THR | L | 109 | 36.625 | −0.517 | 17.184 | 1.00 | 21.88 | O |
| ATOM | 2539 | CG2 | THR | L | 109 | 38.710 | 0.649 | 17.604 | 1.00 | 21.18 | C |
| ATOM | 2540 | C | THR | L | 109 | 36.925 | −2.142 | 19.510 | 1.00 | 19.62 | C |
| ATOM | 2541 | O | THR | L | 109 | 36.271 | −3.027 | 18.937 | 1.00 | 19.24 | O |
| ATOM | 2542 | N | VAL | L | 110 | 36.651 | −1.693 | 20.732 | 1.00 | 19.12 | N |
| ATOM | 2543 | CA | VAL | L | 110 | 35.540 | −2.195 | 21.527 | 1.00 | 18.94 | C |
| ATOM | 2544 | CB | VAL | L | 110 | 35.537 | −1.570 | 22.951 | 1.00 | 19.18 | C |
| ATOM | 2545 | CG1 | VAL | L | 110 | 34.319 | −2.035 | 23.764 | 1.00 | 19.42 | C |
| ATOM | 2546 | CG2 | VAL | L | 110 | 36.832 | −1.902 | 23.692 | 1.00 | 17.27 | C |
| ATOM | 2547 | C | VAL | L | 110 | 34.224 | −1.898 | 20.814 | 1.00 | 19.02 | C |
| ATOM | 2548 | O | VAL | L | 110 | 34.008 | −0.788 | 20.351 | 1.00 | 19.30 | O |
| ATOM | 2549 | N | ALA | L | 111 | 33.362 | −2.904 | 20.705 | 1.00 | 18.68 | N |
| ATOM | 2550 | CA | ALA | L | 111 | 32.037 | −2.728 | 20.128 | 1.00 | 18.90 | C |
| ATOM | 2551 | CB | ALA | L | 111 | 31.966 | −3.340 | 18.740 | 1.00 | 19.16 | C |
| ATOM | 2552 | C | ALA | L | 111 | 30.988 | −3.336 | 21.046 | 1.00 | 18.68 | C |
| ATOM | 2553 | O | ALA | L | 111 | 31.066 | −4.509 | 21.400 | 1.00 | 18.58 | O |
| ATOM | 2554 | N | ALA | L | 112 | 30.025 | −2.520 | 21.460 | 1.00 | 18.97 | N |
| ATOM | 2555 | CA | ALA | L | 112 | 28.940 | −2.982 | 22.320 | 1.00 | 19.03 | C |
| ATOM | 2556 | CB | ALA | L | 112 | 28.171 | −1.785 | 22.905 | 1.00 | 19.36 | C |
| ATOM | 2557 | C | ALA | L | 112 | 28.003 | −3.914 | 21.546 | 1.00 | 19.25 | C |
| ATOM | 2558 | O | ALA | L | 112 | 27.759 | −3.696 | 20.355 | 1.00 | 19.45 | O |
| ATOM | 2559 | N | PRO | L | 113 | 27.478 | −4.960 | 22.211 | 1.00 | 19.68 | N |
| ATOM | 2560 | CA | PRO | L | 113 | 26.458 | −5.792 | 21.562 | 1.00 | 19.87 | C |
| ATOM | 2561 | CB | PRO | L | 113 | 26.224 | −6.913 | 22.577 | 1.00 | 20.17 | C |
| ATOM | 2562 | CG | PRO | L | 113 | 26.603 | −6.298 | 23.905 | 1.00 | 20.46 | C |
| ATOM | 2563 | CD | PRO | L | 113 | 27.807 | −5.460 | 23.561 | 1.00 | 19.65 | C |
| ATOM | 2564 | C | PRO | L | 113 | 25.134 | −5.058 | 21.331 | 1.00 | 20.01 | C |
| ATOM | 2565 | O | PRO | L | 113 | 24.746 | −4.195 | 22.127 | 1.00 | 20.39 | O |
| ATOM | 2566 | N | SER | L | 114 | 24.467 | −5.402 | 20.237 | 1.00 | 20.02 | N |
| ATOM | 2567 | CA | SER | L | 114 | 23.069 | −5.092 | 20.053 | 1.00 | 20.51 | C |
| ATOM | 2568 | CB | SER | L | 114 | 22.751 | −4.957 | 18.562 | 1.00 | 21.47 | C |
| ATOM | 2569 | OG | SER | L | 114 | 23.422 | −3.830 | 18.015 | 1.00 | 23.37 | O |
| ATOM | 2570 | C | SER | L | 114 | 22.304 | −6.251 | 20.683 | 1.00 | 19.73 | C |
| ATOM | 2571 | O | SER | L | 114 | 22.553 | −7.410 | 20.346 | 1.00 | 19.57 | O |
| ATOM | 2572 | N | VAL | L | 115 | 21.412 | −5.947 | 21.624 | 1.00 | 18.30 | N |
| ATOM | 2573 | CA | VAL | L | 115 | 20.706 | −6.995 | 22.361 | 1.00 | 17.31 | C |
| ATOM | 2574 | CB | VAL | L | 115 | 20.782 | −6.756 | 23.895 | 1.00 | 17.20 | C |
| ATOM | 2575 | CG1 | VAL | L | 115 | 20.148 | −7.909 | 24.670 | 1.00 | 16.82 | C |
| ATOM | 2576 | CG2 | VAL | L | 115 | 22.239 | −6.573 | 24.333 | 1.00 | 16.85 | C |
| ATOM | 2577 | C | VAL | L | 115 | 19.258 | −7.180 | 21.888 | 1.00 | 16.86 | C |
| ATOM | 2578 | O | VAL | L | 115 | 18.538 | −6.202 | 21.678 | 1.00 | 17.32 | O |
| ATOM | 2579 | N | PHE | L | 116 | 18.831 | −8.431 | 21.733 | 1.00 | 16.01 | N |
| ATOM | 2580 | CA | PHE | L | 116 | 17.455 | −8.725 | 21.348 | 1.00 | 16.82 | C |
| ATOM | 2581 | CB | PHE | L | 116 | 17.356 | −9.135 | 19.870 | 1.00 | 17.32 | C |
| ATOM | 2582 | CG | PHE | L | 116 | 17.911 | −8.124 | 18.907 | 1.00 | 17.34 | C |
| ATOM | 2583 | CD1 | PHE | L | 116 | 17.075 | −7.183 | 18.305 | 1.00 | 19.62 | C |
| ATOM | 2584 | CE1 | PHE | L | 116 | 17.585 | −6.244 | 17.400 | 1.00 | 19.54 | C |
| ATOM | 2585 | CZ | PHE | L | 116 | 18.949 | −6.247 | 17.096 | 1.00 | 17.22 | C |
| ATOM | 2586 | CE2 | PHE | L | 116 | 19.787 | −7.182 | 17.692 | 1.00 | 18.09 | C |
| ATOM | 2587 | CD2 | PHE | L | 116 | 19.260 | −8.115 | 18.596 | 1.00 | 17.64 | C |
| ATOM | 2588 | C | PHE | L | 116 | 16.907 | −9.848 | 22.195 | 1.00 | 16.76 | C |
| ATOM | 2589 | O | PHE | L | 116 | 17.605 | −10.809 | 22.456 | 1.00 | 15.85 | O |
| ATOM | 2590 | N | ILE | L | 117 | 15.645 | −9.728 | 22.610 | 1.00 | 16.76 | N |
| ATOM | 2591 | CA | ILE | L | 117 | 14.970 | −10.821 | 23.309 | 1.00 | 17.41 | C |
| ATOM | 2592 | CB | ILE | L | 117 | 14.502 | −10.427 | 24.761 | 1.00 | 17.46 | C |
| ATOM | 2593 | CG1 | ILE | L | 117 | 14.063 | −11.669 | 25.558 | 1.00 | 17.22 | C |
| ATOM | 2594 | CD1 | ILE | L | 117 | 13.904 | −11.439 | 27.075 | 1.00 | 17.27 | C |
| ATOM | 2595 | CG2 | ILE | L | 117 | 13.400 | −9.343 | 24.726 | 1.00 | 17.90 | C |
| ATOM | 2596 | C | ILE | L | 117 | 13.823 | −11.362 | 22.455 | 1.00 | 17.84 | C |
| ATOM | 2597 | O | ILE | L | 117 | 13.145 | −10.603 | 21.743 | 1.00 | 17.97 | O |
| ATOM | 2598 | N | PHE | L | 118 | 13.633 | −12.678 | 22.507 | 1.00 | 18.11 | N |
| ATOM | 2599 | CA | PHE | L | 118 | 12.554 | −13.338 | 21.782 | 1.00 | 18.50 | C |
| ATOM | 2600 | CB | PHE | L | 118 | 13.103 | −14.221 | 20.665 | 1.00 | 18.78 | C |
| ATOM | 2601 | CG | PHE | L | 118 | 14.002 | −13.502 | 19.698 | 1.00 | 19.13 | C |
| ATOM | 2602 | CD1 | PHE | L | 118 | 13.470 | −12.846 | 18.595 | 1.00 | 19.23 | C |
| ATOM | 2603 | CE1 | PHE | L | 118 | 14.292 | −12.190 | 17.695 | 1.00 | 19.24 | C |
| ATOM | 2604 | CZ | PHE | L | 118 | 15.673 | −12.189 | 17.887 | 1.00 | 20.64 | C |
| ATOM | 2605 | CE2 | PHE | L | 118 | 16.221 | −12.843 | 18.987 | 1.00 | 20.30 | C |
| ATOM | 2606 | CD2 | PHE | L | 118 | 15.379 | −13.496 | 19.884 | 1.00 | 19.27 | C |
| ATOM | 2607 | C | PHE | L | 118 | 11.731 | −14.191 | 22.746 | 1.00 | 18.57 | C |
| ATOM | 2608 | O | PHE | L | 118 | 12.268 | −15.116 | 23.378 | 1.00 | 18.57 | O |
| ATOM | 2609 | N | PRO | L | 119 | 10.426 | −13.891 | 22.863 | 1.00 | 18.57 | N |
| ATOM | 2610 | CA | PRO | L | 119 | 9.563 | −14.753 | 23.659 | 1.00 | 18.45 | C |
| ATOM | 2611 | CB | PRO | L | 119 | 8.204 | −14.037 | 23.619 | 1.00 | 18.72 | C |
| ATOM | 2612 | CG | PRO | L | 119 | 8.499 | −12.634 | 23.179 | 1.00 | 19.29 | C |
| ATOM | 2613 | CD | PRO | L | 119 | 9.692 | −12.757 | 22.276 | 1.00 | 18.42 | C |
| ATOM | 2614 | C | PRO | L | 119 | 9.450 | −16.109 | 22.978 | 1.00 | 18.32 | C |
| ATOM | 2615 | O | PRO | L | 119 | 9.726 | −16.202 | 21.796 | 1.00 | 17.90 | O |
| ATOM | 2616 | N | PRO | L | 120 | 9.044 | −17.154 | 23.718 | 1.00 | 18.56 | N |
| ATOM | 2617 | CA | PRO | L | 120 | 8.700 | −18.413 | 23.061 | 1.00 | 18.65 | C |
| ATOM | 2618 | CB | PRO | L | 120 | 8.249 | −19.305 | 24.228 | 1.00 | 18.61 | C |
| ATOM | 2619 | CG | PRO | L | 120 | 7.879 | −18.354 | 25.312 | 1.00 | 18.68 | C |
| ATOM | 2620 | CD | PRO | L | 120 | 8.879 | −17.247 | 25.180 | 1.00 | 18.55 | C |
| ATOM | 2621 | C | PRO | L | 120 | 7.536 | −18.236 | 22.092 | 1.00 | 19.19 | C |
| ATOM | 2622 | O | PRO | L | 120 | 6.629 | −17.425 | 22.341 | 1.00 | 18.86 | O |
| ATOM | 2623 | N | SER | L | 121 | 7.549 | −19.019 | 21.020 | 1.00 | 19.11 | N |
| ATOM | 2624 | CA | SER | L | 121 | 6.470 | −19.012 | 20.044 | 1.00 | 19.85 | C |
| ATOM | 2625 | CB | SER | L | 121 | 6.949 | −19.651 | 18.733 | 1.00 | 19.97 | C |
| ATOM | 2626 | OG | SER | L | 121 | 7.209 | −21.036 | 18.902 | 1.00 | 18.24 | O |
| ATOM | 2627 | C | SER | L | 121 | 5.224 | −19.737 | 20.567 | 1.00 | 20.36 | C |
| ATOM | 2628 | O | SER | L | 121 | 5.317 | −20.644 | 21.400 | 1.00 | 19.77 | O |
| ATOM | 2629 | N | ASP | L | 122 | 4.056 | −19.328 | 20.075 | 1.00 | 21.69 | N |
| ATOM | 2630 | CA | ASP | L | 122 | 2.812 | −20.046 | 20.340 | 1.00 | 22.47 | C |
| ATOM | 2631 | CB | ASP | L | 122 | 1.650 | −19.399 | 19.576 | 1.00 | 23.40 | C |
| ATOM | 2632 | CG | ASP | L | 122 | 1.333 | −18.000 | 20.085 | 1.00 | 25.99 | C |
| ATOM | 2633 | OD1 | ASP | L | 122 | 0.282 | −17.834 | 20.726 | 1.00 | 28.28 | O |
| ATOM | 2634 | OD2 | ASP | L | 122 | 2.155 | −17.076 | 19.878 | 1.00 | 30.45 | O |
| ATOM | 2635 | C | ASP | L | 122 | 2.953 | −21.524 | 19.976 | 1.00 | 22.30 | C |
| ATOM | 2636 | O | ASP | L | 122 | 2.509 | −22.396 | 20.723 | 1.00 | 21.92 | O |
| ATOM | 2637 | N | GLU | L | 123 | 3.609 | −21.785 | 18.842 | 1.00 | 22.51 | N |
| ATOM | 2638 | CA | GLU | L | 123 | 3.847 | −23.141 | 18.349 | 1.00 | 22.86 | C |
| ATOM | 2639 | CB | GLU | L | 123 | 4.664 | −23.110 | 17.051 | 1.00 | 23.18 | C |
| ATOM | 2640 | CG | GLU | L | 123 | 4.200 | −24.148 | 16.034 | 1.00 | 26.67 | C |
| ATOM | 2641 | CD | GLU | L | 123 | 5.327 | −24.749 | 15.196 | 1.00 | 30.30 | C |
| ATOM | 2642 | OE1 | GLU | L | 123 | 6.279 | −24.028 | 14.804 | 1.00 | 32.89 | O |
| ATOM | 2643 | OE2 | GLU | L | 123 | 5.254 | −25.968 | 14.914 | 1.00 | 32.37 | O |
| ATOM | 2644 | C | GLU | L | 123 | 4.555 | −24.022 | 19.375 | 1.00 | 22.09 | C |
| ATOM | 2645 | O | GLU | L | 123 | 4.119 | −25.143 | 19.655 | 1.00 | 21.98 | O |
| ATOM | 2646 | N | GLN | L | 124 | 5.654 | −23.509 | 19.926 | 1.00 | 21.47 | N |
| ATOM | 2647 | CA | GLN | L | 124 | 6.410 | −24.229 | 20.931 | 1.00 | 20.74 | C |
| ATOM | 2648 | CB | GLN | L | 124 | 7.692 | −23.489 | 21.293 | 1.00 | 20.10 | C |
| ATOM | 2649 | CG | GLN | L | 124 | 8.618 | −24.380 | 22.083 | 1.00 | 18.71 | C |
| ATOM | 2650 | CD | GLN | L | 124 | 9.846 | −23.684 | 22.616 | 1.00 | 17.27 | C |
| ATOM | 2651 | OE1 | GLN | L | 124 | 9.922 | −22.452 | 22.669 | 1.00 | 16.53 | O |
| ATOM | 2652 | NE2 | GLN | L | 124 | 10.824 | −24.481 | 23.022 | 1.00 | 15.02 | N |
| ATOM | 2653 | C | GLN | L | 124 | 5.585 | −24.455 | 22.186 | 1.00 | 21.16 | C |
| ATOM | 2654 | O | GLN | L | 124 | 5.584 | −25.554 | 22.739 | 1.00 | 21.04 | O |
| ATOM | 2655 | N | LEU | L | 125 | 4.881 | −23.418 | 22.630 | 1.00 | 21.65 | N |
| ATOM | 2656 | CA | LEU | L | 125 | 4.135 | −23.505 | 23.888 | 1.00 | 22.80 | C |
| ATOM | 2657 | CB | LEU | L | 125 | 3.450 | −22.172 | 24.234 | 1.00 | 22.85 | C |
| ATOM | 2658 | CG | LEU | L | 125 | 4.419 | −21.066 | 24.697 | 1.00 | 22.86 | C |
| ATOM | 2659 | CD1 | LEU | L | 125 | 5.178 | −21.472 | 25.958 | 1.00 | 22.94 | C |
| ATOM | 2660 | CD2 | LEU | L | 125 | 3.719 | −19.744 | 24.909 | 1.00 | 23.07 | C |
| ATOM | 2661 | C | LEU | L | 125 | 3.152 | −24.668 | 23.892 | 1.00 | 23.78 | C |
| ATOM | 2662 | O | LEU | L | 125 | 3.007 | −25.356 | 24.907 | 1.00 | 24.15 | O |
| ATOM | 2663 | N | LYS | L | 126 | 2.525 | −24.909 | 22.738 | 1.00 | 24.70 | N |
| ATOM | 2664 | CA | LYS | L | 126 | 1.577 | −26.010 | 22.563 | 1.00 | 25.73 | C |
| ATOM | 2665 | CB | LYS | L | 126 | 0.935 | −25.972 | 21.172 | 1.00 | 25.73 | C |
| ATOM | 2666 | CG | LYS | L | 126 | −0.236 | −24.987 | 21.049 | 1.00 | 26.88 | C |
| ATOM | 2667 | CD | LYS | L | 126 | −1.414 | −25.549 | 20.224 | 1.00 | 28.33 | C |
| ATOM | 2668 | CE | LYS | L | 126 | −0.965 | −26.295 | 18.963 | 1.00 | 28.95 | C |
| ATOM | 2669 | NZ | LYS | L | 126 | 0.008 | −25.523 | 18.138 | 1.00 | 28.05 | N |
| ATOM | 2670 | C | LYS | L | 126 | 2.172 | −27.396 | 22.821 | 1.00 | 26.00 | C |
| ATOM | 2671 | O | LYS | L | 126 | 1.446 | −28.308 | 23.236 | 1.00 | 26.63 | O |
| ATOM | 2672 | N | SER | L | 127 | 3.475 | −27.542 | 22.582 | 1.00 | 26.21 | N |
| ATOM | 2673 | CA | SER | L | 127 | 4.184 | −28.815 | 22.782 | 1.00 | 26.27 | C |
| ATOM | 2674 | CB | SER | L | 127 | 5.346 | −28.960 | 21.791 | 1.00 | 26.05 | C |
| ATOM | 2675 | OG | SER | L | 127 | 6.478 | −28.194 | 22.185 | 1.00 | 28.13 | O |
| ATOM | 2676 | C | SER | L | 127 | 4.667 | −29.046 | 24.223 | 1.00 | 25.77 | C |
| ATOM | 2677 | O | SER | L | 127 | 5.225 | −30.107 | 24.529 | 1.00 | 25.86 | O |
| ATOM | 2678 | N | GLY | L | 128 | 4.462 | −28.056 | 25.094 | 1.00 | 25.03 | N |
| ATOM | 2679 | CA | GLY | L | 128 | 4.702 | −28.219 | 26.528 | 1.00 | 24.10 | C |
| ATOM | 2680 | C | GLY | L | 128 | 5.993 | −27.637 | 27.075 | 1.00 | 23.61 | C |
| ATOM | 2681 | O | GLY | L | 128 | 6.351 | −27.875 | 28.232 | 1.00 | 23.68 | O |
| ATOM | 2682 | N | THR | L | 129 | 6.701 | −26.867 | 26.260 | 1.00 | 22.53 | N |
| ATOM | 2683 | CA | THR | L | 129 | 7.955 | −26.291 | 26.723 | 1.00 | 22.21 | C |
| ATOM | 2684 | CB | THR | L | 129 | 9.149 | −27.261 | 26.489 | 1.00 | 22.12 | C |
| ATOM | 2685 | OG1 | THR | L | 129 | 10.310 | −26.775 | 27.175 | 1.00 | 25.34 | O |
| ATOM | 2686 | CG2 | THR | L | 129 | 9.452 | −27.436 | 25.005 | 1.00 | 23.48 | C |
| ATOM | 2687 | C | THR | L | 129 | 8.172 | −24.878 | 26.176 | 1.00 | 20.79 | C |
| ATOM | 2688 | O | THR | L | 129 | 7.640 | −24.525 | 25.131 | 1.00 | 21.39 | O |
| ATOM | 2689 | N | ALA | L | 130 | 8.892 | −24.054 | 26.928 | 1.00 | 19.45 | N |
| ATOM | 2690 | CA | ALA | L | 130 | 9.143 | −22.670 | 26.542 | 1.00 | 17.98 | C |
| ATOM | 2691 | CB | ALA | L | 130 | 8.517 | −21.719 | 27.536 | 1.00 | 17.63 | C |
| ATOM | 2692 | C | ALA | L | 130 | 10.635 | −22.413 | 26.444 | 1.00 | 17.23 | C |
| ATOM | 2693 | O | ALA | L | 130 | 11.367 | −22.662 | 27.404 | 1.00 | 16.69 | O |
| ATOM | 2694 | N | SER | L | 131 | 11.085 | −21.948 | 25.275 | 1.00 | 16.12 | N |
| ATOM | 2695 | CA | SER | L | 131 | 12.445 | −21.433 | 25.137 | 1.00 | 15.55 | C |
| ATOM | 2696 | CB | SER | L | 131 | 13.186 | −22.110 | 23.976 | 1.00 | 15.59 | C |
| ATOM | 2697 | OG | SER | L | 131 | 13.157 | −23.524 | 24.087 | 1.00 | 13.67 | O |
| ATOM | 2698 | C | SER | L | 131 | 12.408 | −19.914 | 24.949 | 1.00 | 15.53 | C |
| ATOM | 2699 | O | SER | L | 131 | 11.670 | −19.397 | 24.104 | 1.00 | 15.54 | O |
| ATOM | 2700 | N | VAL | L | 132 | 13.198 | −19.208 | 25.752 | 1.00 | 14.79 | N |
| ATOM | 2701 | CA | VAL | L | 132 | 13.332 | −17.760 | 25.661 | 1.00 | 14.26 | C |
| ATOM | 2702 | CB | VAL | L | 132 | 13.029 | −17.085 | 27.033 | 1.00 | 14.22 | C |
| ATOM | 2703 | CG1 | VAL | L | 132 | 12.872 | −15.579 | 26.887 | 1.00 | 13.36 | C |
| ATOM | 2704 | CG2 | VAL | L | 132 | 11.756 | −17.678 | 27.661 | 1.00 | 14.49 | C |
| ATOM | 2705 | C | VAL | L | 132 | 14.757 | −17.432 | 25.188 | 1.00 | 14.19 | C |
| ATOM | 2706 | O | VAL | L | 132 | 15.740 | −17.850 | 25.818 | 1.00 | 13.85 | O |
| ATOM | 2707 | N | VAL | L | 133 | 14.881 | −16.703 | 24.083 | 1.00 | 13.76 | N |
| ATOM | 2708 | CA | VAL | L | 133 | 16.201 | −16.448 | 23.510 | 1.00 | 14.48 | C |
| ATOM | 2709 | CB | VAL | L | 133 | 16.299 | −16.886 | 22.024 | 1.00 | 14.28 | C |
| ATOM | 2710 | CG1 | VAL | L | 133 | 17.680 | −16.548 | 21.442 | 1.00 | 14.32 | C |
| ATOM | 2711 | CG2 | VAL | L | 133 | 16.034 | −18.366 | 21.913 | 1.00 | 14.96 | C |
| ATOM | 2712 | C | VAL | L | 133 | 16.637 | −15.005 | 23.644 | 1.00 | 14.79 | C |
| ATOM | 2713 | O | VAL | L | 133 | 15.862 | −14.083 | 23.385 | 1.00 | 14.67 | O |
| ATOM | 2714 | N | CYS | L | 134 | 17.885 | −14.830 | 24.071 | 1.00 | 15.09 | N |
| ATOM | 2715 | CA | CYS | L | 134 | 18.501 | −13.516 | 24.155 | 1.00 | 16.36 | C |
| ATOM | 2716 | CB | CYS | L | 134 | 18.952 | −13.236 | 25.588 | 1.00 | 17.33 | C |
| ATOM | 2717 | SG | CYS | L | 134 | 19.689 | −11.595 | 25.862 | 1.00 | 19.82 | S |
| ATOM | 2718 | C | CYS | L | 134 | 19.691 | −13.483 | 23.200 | 1.00 | 16.26 | C |
| ATOM | 2719 | O | CYS | L | 134 | 20.565 | −14.358 | 23.260 | 1.00 | 16.24 | O |
| ATOM | 2720 | N | LEU | L | 135 | 19.704 | −12.501 | 22.300 | 1.00 | 16.04 | N |
| ATOM | 2721 | CA | LEU | L | 135 | 20.768 | −12.401 | 21.290 | 1.00 | 15.55 | C |
| ATOM | 2722 | CB | LEU | L | 135 | 20.179 | −12.277 | 19.871 | 1.00 | 15.45 | C |
| ATOM | 2723 | CG | LEU | L | 135 | 21.136 | −11.864 | 18.747 | 1.00 | 14.30 | C |
| ATOM | 2724 | CD1 | LEU | L | 135 | 22.131 | −12.979 | 18.476 | 1.00 | 14.81 | C |
| ATOM | 2725 | CD2 | LEU | L | 135 | 20.384 | −11.519 | 17.460 | 1.00 | 15.42 | C |
| ATOM | 2726 | C | LEU | L | 135 | 21.643 | −11.201 | 21.573 | 1.00 | 15.75 | C |
| ATOM | 2727 | O | LEU | L | 135 | 21.140 | −10.092 | 21.752 | 1.00 | 15.61 | O |
| ATOM | 2728 | N | LEU | L | 136 | 22.948 | −11.441 | 21.627 | 1.00 | 16.00 | N |
| ATOM | 2729 | CA | LEU | L | 136 | 23.958 | −10.390 | 21.708 | 1.00 | 16.06 | C |
| ATOM | 2730 | CB | LEU | L | 136 | 24.969 | −10.678 | 22.830 | 1.00 | 15.28 | C |
| ATOM | 2731 | CG | LEU | L | 136 | 24.565 | −10.457 | 24.297 | 1.00 | 15.07 | C |
| ATOM | 2732 | CD1 | LEU | L | 136 | 23.374 | −11.285 | 24.698 | 1.00 | 15.20 | C |
| ATOM | 2733 | CD2 | LEU | L | 136 | 25.743 | −10.766 | 25.202 | 1.00 | 18.18 | C |
| ATOM | 2734 | C | LEU | L | 136 | 24.669 | −10.411 | 20.374 | 1.00 | 16.54 | C |
| ATOM | 2735 | O | LEU | L | 136 | 25.413 | −11.346 | 20.084 | 1.00 | 16.96 | O |
| ATOM | 2736 | N | ASN | L | 137 | 24.451 | −9.379 | 19.572 | 1.00 | 16.90 | N |
| ATOM | 2737 | CA | ASN | L | 137 | 24.941 | −9.362 | 18.207 | 1.00 | 17.48 | C |
| ATOM | 2738 | CB | ASN | L | 137 | 23.809 | −9.002 | 17.240 | 1.00 | 17.72 | C |
| ATOM | 2739 | CG | ASN | L | 137 | 23.997 | −9.627 | 15.883 | 1.00 | 19.28 | C |
| ATOM | 2740 | OD1 | ASN | L | 137 | 24.284 | −10.819 | 15.775 | 1.00 | 21.31 | O |
| ATOM | 2741 | ND2 | ASN | L | 137 | 23.824 | −8.835 | 14.832 | 1.00 | 19.76 | N |
| ATOM | 2742 | C | ASN | L | 137 | 26.117 | −8.427 | 17.992 | 1.00 | 17.77 | C |
| ATOM | 2743 | O | ASN | L | 137 | 26.091 | −7.282 | 18.448 | 1.00 | 18.32 | O |
| ATOM | 2744 | N | ASN | L | 138 | 27.146 | −8.948 | 17.322 | 1.00 | 17.42 | N |
| ATOM | 2745 | CA | ASN | L | 138 | 28.264 | −8.169 | 16.775 | 1.00 | 17.47 | C |
| ATOM | 2746 | CB | ASN | L | 138 | 27.802 | −7.355 | 15.558 | 1.00 | 18.13 | C |
| ATOM | 2747 | CG | ASN | L | 138 | 27.362 | −8.241 | 14.383 | 1.00 | 19.93 | C |
| ATOM | 2748 | OD1 | ASN | L | 138 | 27.529 | −9.462 | 14.402 | 1.00 | 21.13 | O |
| ATOM | 2749 | ND2 | ASN | L | 138 | 26.810 | −7.613 | 13.351 | 1.00 | 20.47 | N |
| ATOM | 2750 | C | ASN | L | 138 | 29.064 | −7.307 | 17.780 | 1.00 | 17.19 | C |
| ATOM | 2751 | O | ASN | L | 138 | 29.193 | −6.090 | 17.631 | 1.00 | 16.42 | O |
| ATOM | 2752 | N | PHE | L | 139 | 29.602 | −7.956 | 18.798 | 1.00 | 16.18 | N |
| ATOM | 2753 | CA | PHE | L | 139 | 30.301 | −7.260 | 19.862 | 1.00 | 16.70 | C |
| ATOM | 2754 | CB | PHE | L | 139 | 29.621 | −7.520 | 21.216 | 1.00 | 16.56 | C |
| ATOM | 2755 | CG | PHE | L | 139 | 29.601 | −8.974 | 21.615 | 1.00 | 16.95 | C |
| ATOM | 2756 | CD1 | PHE | L | 139 | 28.557 | −9.808 | 21.211 | 1.00 | 15.34 | C |
| ATOM | 2757 | CE1 | PHE | L | 139 | 28.544 | −11.161 | 21.577 | 1.00 | 17.21 | C |
| ATOM | 2758 | CZ | PHE | L | 139 | 29.580 | −11.685 | 22.354 | 1.00 | 15.61 | C |
| ATOM | 2759 | CE2 | PHE | L | 139 | 30.628 | −10.857 | 22.758 | 1.00 | 16.15 | C |
| ATOM | 2760 | CD2 | PHE | L | 139 | 30.627 | −9.506 | 22.383 | 1.00 | 15.05 | C |
| ATOM | 2761 | C | PHE | L | 139 | 31.778 | −7.644 | 19.900 | 1.00 | 16.87 | C |
| ATOM | 2762 | O | PHE | L | 139 | 32.190 | −8.668 | 19.326 | 1.00 | 17.12 | O |
| ATOM | 2763 | N | TYR | L | 140 | 32.579 | −6.803 | 20.551 | 1.00 | 16.97 | N |
| ATOM | 2764 | CA | TYR | L | 140 | 33.996 | −7.093 | 20.752 | 1.00 | 17.10 | C |
| ATOM | 2765 | CB | TYR | L | 140 | 34.853 | −6.753 | 19.512 | 1.00 | 16.53 | C |
| ATOM | 2766 | CG | TYR | L | 140 | 36.251 | −7.236 | 19.733 | 1.00 | 17.06 | C |
| ATOM | 2767 | CD1 | TYR | L | 140 | 37.187 | −6.431 | 20.384 | 1.00 | 18.91 | C |
| ATOM | 2768 | CE1 | TYR | L | 140 | 38.477 | −6.911 | 20.663 | 1.00 | 18.82 | C |
| ATOM | 2769 | CZ | TYR | L | 140 | 38.812 | −8.201 | 20.296 | 1.00 | 17.85 | C |
| ATOM | 2770 | OH | TYR | L | 140 | 40.071 | −8.670 | 20.580 | 1.00 | 20.31 | O |
| ATOM | 2771 | CE2 | TYR | L | 140 | 37.891 | −9.026 | 19.667 | 1.00 | 17.84 | C |
| ATOM | 2772 | CD2 | TYR | L | 140 | 36.615 | −8.545 | 19.401 | 1.00 | 16.43 | C |
| ATOM | 2773 | C | TYR | L | 140 | 34.497 | −6.332 | 21.974 | 1.00 | 17.11 | C |
| ATOM | 2774 | O | TYR | L | 140 | 34.190 | −5.145 | 22.100 | 1.00 | 17.62 | O |
| ATOM | 2775 | N | PRO | L | 141 | 35.274 | −6.986 | 22.871 | 1.00 | 17.66 | N |
| ATOM | 2776 | CA | PRO | L | 141 | 35.821 | −8.360 | 22.850 | 1.00 | 18.37 | C |
| ATOM | 2777 | CB | PRO | L | 141 | 36.982 | −8.289 | 23.860 | 1.00 | 18.49 | C |
| ATOM | 2778 | CG | PRO | L | 141 | 36.471 | −7.269 | 24.908 | 1.00 | 18.03 | C |
| ATOM | 2779 | CD | PRO | L | 141 | 35.623 | −6.273 | 24.120 | 1.00 | 17.38 | C |
| ATOM | 2780 | C | PRO | L | 141 | 34.807 | −9.439 | 23.261 | 1.00 | 19.41 | C |
| ATOM | 2781 | O | PRO | L | 141 | 33.642 | −9.125 | 23.551 | 1.00 | 19.38 | O |
| ATOM | 2782 | N | ARG | L | 142 | 35.247 | −10.698 | 23.288 | 1.00 | 20.44 | N |
| ATOM | 2783 | CA | ARG | L | 142 | 34.337 | −11.835 | 23.528 | 1.00 | 22.23 | C |
| ATOM | 2784 | CB | ARG | L | 142 | 34.996 | −13.189 | 23.187 | 1.00 | 23.18 | C |
| ATOM | 2785 | CG | ARG | L | 142 | 34.098 | −14.428 | 23.483 | 1.00 | 28.05 | C |
| ATOM | 2786 | CD | ARG | L | 142 | 34.642 | −15.709 | 22.847 | 1.00 | 38.23 | C |
| ATOM | 2787 | NE | ARG | L | 142 | 33.814 | −16.882 | 23.170 | 1.00 | 44.66 | N |
| ATOM | 2788 | CZ | ARG | L | 142 | 34.191 | −17.898 | 23.957 | 1.00 | 47.20 | C |
| ATOM | 2789 | NH1 | ARG | L | 142 | 35.398 | −17.909 | 24.519 | 1.00 | 48.50 | N |
| ATOM | 2790 | NH2 | ARG | L | 142 | 33.361 | −18.918 | 24.179 | 1.00 | 48.03 | N |
| ATOM | 2791 | C | ARG | L | 142 | 33.742 | −11.890 | 24.939 | 1.00 | 22.55 | C |
| ATOM | 2792 | O | ARG | L | 142 | 32.604 | −12.355 | 25.100 | 1.00 | 22.30 | O |
| ATOM | 2793 | N | GLU | L | 143 | 34.494 | −11.446 | 25.953 | 1.00 | 22.79 | N |
| ATOM | 2794 | CA | GLU | L | 143 | 33.977 | −11.453 | 27.330 | 1.00 | 23.78 | C |
| ATOM | 2795 | CB | GLU | L | 143 | 34.970 | −10.850 | 28.327 | 1.00 | 24.53 | C |
| ATOM | 2796 | CG | GLU | L | 143 | 34.510 | −11.023 | 29.804 | 1.00 | 28.41 | C |
| ATOM | 2797 | CD | GLU | L | 143 | 35.399 | −10.316 | 30.830 | 1.00 | 33.71 | C |
| ATOM | 2798 | OE1 | GLU | L | 143 | 36.617 | −10.148 | 30.579 | 1.00 | 35.88 | O |
| ATOM | 2799 | OE2 | GLU | L | 143 | 34.872 | −9.940 | 31.905 | 1.00 | 35.42 | O |
| ATOM | 2800 | C | GLU | L | 143 | 32.636 | −10.722 | 27.421 | 1.00 | 23.52 | C |
| ATOM | 2801 | O | GLU | L | 143 | 32.528 | −9.559 | 27.017 | 1.00 | 22.75 | O |
| ATOM | 2802 | N | ALA | L | 144 | 31.621 | −11.423 | 27.927 | 1.00 | 22.99 | N |
| ATOM | 2803 | CA | ALA | L | 144 | 30.279 | −10.865 | 28.114 | 1.00 | 23.13 | C |
| ATOM | 2804 | CB | ALA | L | 144 | 29.493 | −10.923 | 26.815 | 1.00 | 22.87 | C |
| ATOM | 2805 | C | ALA | L | 144 | 29.540 | −11.626 | 29.213 | 1.00 | 23.33 | C |
| ATOM | 2806 | O | ALA | L | 144 | 29.731 | −12.831 | 29.380 | 1.00 | 24.03 | O |
| ATOM | 2807 | N | LYS | L | 145 | 28.714 | −10.926 | 29.977 | 1.00 | 23.45 | N |
| ATOM | 2808 | CA | LYS | L | 145 | 27.902 | −11.602 | 30.975 | 1.00 | 23.10 | C |
| ATOM | 2809 | CB | LYS | L | 145 | 28.188 | −11.078 | 32.383 | 1.00 | 23.72 | C |
| ATOM | 2810 | CG | LYS | L | 145 | 27.510 | −11.903 | 33.485 | 1.00 | 25.65 | C |
| ATOM | 2811 | CD | LYS | L | 145 | 27.038 | −11.055 | 34.652 | 1.00 | 28.92 | C |
| ATOM | 2812 | CE | LYS | L | 145 | 28.145 | −10.672 | 35.607 | 1.00 | 30.12 | C |
| ATOM | 2813 | NZ | LYS | L | 145 | 27.559 | −10.015 | 36.821 | 1.00 | 31.75 | N |
| ATOM | 2814 | C | LYS | L | 145 | 26.432 | −11.441 | 30.631 | 1.00 | 22.55 | C |
| ATOM | 2815 | O | LYS | L | 145 | 25.955 | −10.327 | 30.386 | 1.00 | 22.32 | O |
| ATOM | 2816 | N | VAL | L | 146 | 25.720 | −12.563 | 30.610 | 1.00 | 21.57 | N |
| ATOM | 2817 | CA | VAL | L | 146 | 24.281 | −12.550 | 30.407 | 1.00 | 21.65 | C |
| ATOM | 2818 | CB | VAL | L | 146 | 23.878 | −13.359 | 29.160 | 1.00 | 21.48 | C |
| ATOM | 2819 | CG1 | VAL | L | 146 | 22.369 | −13.334 | 28.961 | 1.00 | 20.73 | C |
| ATOM | 2820 | CG2 | VAL | L | 146 | 24.602 | −12.836 | 27.922 | 1.00 | 21.44 | C |
| ATOM | 2821 | C | VAL | L | 146 | 23.633 | −13.149 | 31.643 | 1.00 | 21.27 | C |
| ATOM | 2822 | O | VAL | L | 146 | 23.969 | −14.258 | 32.046 | 1.00 | 21.45 | O |
| ATOM | 2823 | N | GLN | L | 147 | 22.726 | −12.401 | 32.250 | 1.00 | 21.48 | N |
| ATOM | 2824 | CA | GLN | L | 147 | 21.944 | −12.903 | 33.380 | 1.00 | 21.59 | C |
| ATOM | 2825 | CB | GLN | L | 147 | 22.194 | −12.074 | 34.635 | 1.00 | 22.04 | C |
| ATOM | 2826 | CG | GLN | L | 147 | 23.532 | −12.325 | 35.318 | 1.00 | 24.83 | C |
| ATOM | 2827 | CD | GLN | L | 147 | 23.543 | −11.832 | 36.749 | 1.00 | 26.97 | C |
| ATOM | 2828 | OE1 | GLN | L | 147 | 22.694 | −11.027 | 37.157 | 1.00 | 30.53 | O |
| ATOM | 2829 | NE2 | GLN | L | 147 | 24.489 | −12.327 | 37.530 | 1.00 | 28.01 | N |
| ATOM | 2830 | C | GLN | L | 147 | 20.456 | −12.893 | 33.059 | 1.00 | 20.87 | C |
| ATOM | 2831 | O | GLN | L | 147 | 19.927 | −11.936 | 32.469 | 1.00 | 20.41 | O |
| ATOM | 2832 | N | TRP | L | 148 | 19.785 | −13.967 | 33.457 | 1.00 | 20.15 | N |
| ATOM | 2833 | CA | TRP | L | 148 | 18.349 | −14.076 | 33.277 | 1.00 | 19.67 | C |
| ATOM | 2834 | CB | TRP | L | 148 | 17.972 | −15.481 | 32.813 | 1.00 | 19.04 | C |
| ATOM | 2835 | CG | TRP | L | 148 | 18.245 | −15.733 | 31.361 | 1.00 | 19.02 | C |
| ATOM | 2836 | CD1 | TRP | L | 148 | 19.316 | −16.406 | 30.831 | 1.00 | 18.16 | C |
| ATOM | 2837 | NE1 | TRP | L | 148 | 19.230 | −16.434 | 29.458 | 1.00 | 18.16 | N |
| ATOM | 2838 | CE2 | TRP | L | 148 | 18.085 | −15.782 | 29.073 | 1.00 | 18.97 | C |
| ATOM | 2839 | CD2 | TRP | L | 148 | 17.435 | −15.328 | 30.251 | 1.00 | 17.33 | C |
| ATOM | 2840 | CE3 | TRP | L | 148 | 16.234 | −14.617 | 30.132 | 1.00 | 17.40 | C |
| ATOM | 2841 | CZ3 | TRP | L | 148 | 15.717 | −14.391 | 28.857 | 1.00 | 17.57 | C |
| ATOM | 2842 | CH2 | TRP | L | 148 | 16.389 | −14.859 | 27.704 | 1.00 | 18.67 | C |
| ATOM | 2843 | CZ2 | TRP | L | 148 | 17.566 | −15.558 | 27.793 | 1.00 | 18.18 | C |
| ATOM | 2844 | C | TRP | L | 148 | 17.640 | −13.732 | 34.589 | 1.00 | 19.84 | C |
| ATOM | 2845 | O | TRP | L | 148 | 17.949 | −14.317 | 35.633 | 1.00 | 19.32 | O |
| ATOM | 2846 | N | LYS | L | 149 | 16.725 | −12.762 | 34.510 | 1.00 | 19.77 | N |
| ATOM | 2847 | CA | LYS | L | 149 | 15.827 | −12.385 | 35.605 | 1.00 | 20.48 | C |
| ATOM | 2848 | CB | LYS | L | 149 | 15.921 | −10.880 | 35.887 | 1.00 | 20.68 | C |
| ATOM | 2849 | CG | LYS | L | 149 | 17.310 | −10.373 | 36.306 | 1.00 | 22.77 | C |
| ATOM | 2850 | CD | LYS | L | 149 | 17.437 | −10.279 | 37.811 | 1.00 | 26.28 | C |
| ATOM | 2851 | CE | LYS | L | 149 | 18.836 | −9.833 | 38.227 | 1.00 | 29.60 | C |
| ATOM | 2852 | NZ | LYS | L | 149 | 18.900 | −9.705 | 39.715 | 1.00 | 31.88 | N |
| ATOM | 2853 | C | LYS | L | 149 | 14.384 | −12.739 | 35.222 | 1.00 | 20.57 | C |
| ATOM | 2854 | O | LYS | L | 149 | 13.914 | −12.354 | 34.143 | 1.00 | 20.49 | O |
| ATOM | 2855 | N | VAL | L | 150 | 13.690 | −13.479 | 36.089 | 1.00 | 20.55 | N |
| ATOM | 2856 | CA | VAL | L | 150 | 12.278 | −13.813 | 35.865 | 1.00 | 20.81 | C |
| ATOM | 2857 | CB | VAL | L | 150 | 12.046 | −15.341 | 35.738 | 1.00 | 20.54 | C |
| ATOM | 2858 | CG1 | VAL | L | 150 | 10.575 | −15.653 | 35.606 | 1.00 | 20.21 | C |
| ATOM | 2859 | CG2 | VAL | L | 150 | 12.785 | −15.909 | 34.549 | 1.00 | 20.94 | C |
| ATOM | 2860 | C | VAL | L | 150 | 11.495 | −13.269 | 37.060 | 1.00 | 21.18 | C |
| ATOM | 2861 | O | VAL | L | 150 | 11.645 | −13.776 | 38.169 | 1.00 | 21.22 | O |
| ATOM | 2862 | N | ASP | L | 151 | 10.667 | −12.250 | 36.817 | 1.00 | 21.74 | N |
| ATOM | 2863 | CA | ASP | L | 151 | 10.030 | −11.435 | 37.874 | 1.00 | 22.40 | C |
| ATOM | 2864 | CB | ASP | L | 151 | 8.856 | −12.171 | 38.562 | 1.00 | 22.09 | C |
| ATOM | 2865 | CG | ASP | L | 151 | 7.595 | −12.230 | 37.702 | 1.00 | 21.90 | C |
| ATOM | 2866 | OD1 | ASP | L | 151 | 7.403 | −11.361 | 36.818 | 1.00 | 21.55 | O |
| ATOM | 2867 | OD2 | ASP | L | 151 | 6.778 | −13.148 | 37.931 | 1.00 | 21.24 | O |
| ATOM | 2868 | C | ASP | L | 151 | 11.047 | −10.964 | 38.908 | 1.00 | 22.76 | C |
| ATOM | 2869 | O | ASP | L | 151 | 10.846 | −11.153 | 40.111 | 1.00 | 23.09 | O |
| ATOM | 2870 | N | ASN | L | 152 | 12.142 | −10.369 | 38.423 | 1.00 | 23.35 | N |
| ATOM | 2871 | CA | ASN | L | 152 | 13.248 | −9.842 | 39.252 | 1.00 | 23.83 | C |
| ATOM | 2872 | CB | ASN | L | 152 | 12.750 | −8.825 | 40.300 | 1.00 | 24.45 | C |
| ATOM | 2873 | CG | ASN | L | 152 | 12.012 | −7.643 | 39.682 | 1.00 | 25.96 | C |
| ATOM | 2874 | OD1 | ASN | L | 152 | 12.055 | −7.421 | 38.466 | 1.00 | 28.38 | O |
| ATOM | 2875 | ND2 | ASN | L | 152 | 11.323 | −6.877 | 40.527 | 1.00 | 28.41 | N |
| ATOM | 2876 | C | ASN | L | 152 | 14.142 | −10.887 | 39.928 | 1.00 | 23.75 | C |
| ATOM | 2877 | O | ASN | L | 152 | 15.143 | −10.527 | 40.549 | 1.00 | 24.09 | O |
| ATOM | 2878 | N | ALA | L | 153 | 13.794 | −12.168 | 39.799 | 1.00 | 23.45 | N |
| ATOM | 2879 | CA | ALA | L | 153 | 14.565 | −13.251 | 40.419 | 1.00 | 22.62 | C |
| ATOM | 2880 | CB | ALA | L | 153 | 13.640 | −14.355 | 40.913 | 1.00 | 23.08 | C |
| ATOM | 2881 | C | ALA | L | 153 | 15.609 | −13.821 | 39.470 | 1.00 | 22.57 | C |
| ATOM | 2882 | O | ALA | L | 153 | 15.300 | −14.180 | 38.328 | 1.00 | 21.50 | O |
| ATOM | 2883 | N | LEU | L | 154 | 16.841 | −13.913 | 39.967 | 1.00 | 22.01 | N |
| ATOM | 2884 | CA | LEU | L | 154 | 17.957 | −14.426 | 39.198 | 1.00 | 22.21 | C |
| ATOM | 2885 | CB | LEU | L | 154 | 19.285 | −14.122 | 39.918 | 1.00 | 22.89 | C |
| ATOM | 2886 | CG | LEU | L | 154 | 20.648 | −14.336 | 39.229 | 1.00 | 24.37 | C |
| ATOM | 2887 | CD1 | LEU | L | 154 | 20.634 | −14.045 | 37.713 | 1.00 | 25.92 | C |
| ATOM | 2888 | CD2 | LEU | L | 154 | 21.237 | −15.724 | 39.514 | 1.00 | 27.37 | C |
| ATOM | 2889 | C | LEU | L | 154 | 17.800 | −15.921 | 38.937 | 1.00 | 21.64 | C |
| ATOM | 2890 | O | LEU | L | 154 | 17.576 | −16.708 | 39.863 | 1.00 | 21.32 | O |
| ATOM | 2891 | N | GLN | L | 155 | 17.911 | −16.292 | 37.663 | 1.00 | 20.83 | N |
| ATOM | 2892 | CA | GLN | L | 155 | 17.820 | −17.682 | 37.231 | 1.00 | 20.45 | C |
| ATOM | 2893 | CB | GLN | L | 155 | 17.295 | −17.742 | 35.791 | 1.00 | 20.46 | C |
| ATOM | 2894 | CG | GLN | L | 155 | 15.887 | −17.173 | 35.664 | 1.00 | 19.78 | C |
| ATOM | 2895 | CD | GLN | L | 155 | 14.901 | −17.899 | 36.552 | 1.00 | 18.71 | C |
| ATOM | 2896 | OE1 | GLN | L | 155 | 14.335 | −17.319 | 37.481 | 1.00 | 17.06 | O |
| ATOM | 2897 | NE2 | GLN | L | 155 | 14.700 | −19.183 | 36.279 | 1.00 | 18.44 | N |
| ATOM | 2898 | C | GLN | L | 155 | 19.162 | −18.381 | 37.294 | 1.00 | 20.39 | C |
| ATOM | 2899 | O | GLN | L | 155 | 20.186 | −17.782 | 36.963 | 1.00 | 20.83 | O |
| ATOM | 2900 | N | SER | L | 156 | 19.168 | −19.643 | 37.714 | 1.00 | 19.71 | N |
| ATOM | 2901 | CA | SER | L | 156 | 20.363 | −20.459 | 37.550 | 1.00 | 20.32 | C |
| ATOM | 2902 | CB | SER | L | 156 | 21.351 | −20.319 | 38.727 | 1.00 | 20.61 | C |
| ATOM | 2903 | OG | SER | L | 156 | 20.936 | −21.014 | 39.873 | 1.00 | 22.62 | O |
| ATOM | 2904 | C | SER | L | 156 | 20.068 | −21.913 | 37.197 | 1.00 | 19.81 | C |
| ATOM | 2905 | O | SER | L | 156 | 19.075 | −22.502 | 37.657 | 1.00 | 19.27 | O |
| ATOM | 2906 | N | GLY | L | 157 | 20.933 | −22.447 | 36.334 | 1.00 | 19.13 | N |
| ATOM | 2907 | CA | GLY | L | 157 | 20.888 | −23.836 | 35.903 | 1.00 | 19.39 | C |
| ATOM | 2908 | C | GLY | L | 157 | 19.883 | −24.152 | 34.809 | 1.00 | 19.39 | C |
| ATOM | 2909 | O | GLY | L | 157 | 19.645 | −25.320 | 34.525 | 1.00 | 20.29 | O |
| ATOM | 2910 | N | ASN | L | 158 | 19.263 | −23.130 | 34.224 | 1.00 | 18.97 | N |
| ATOM | 2911 | CA | ASN | L | 158 | 18.212 | −23.351 | 33.200 | 1.00 | 18.70 | C |
| ATOM | 2912 | CB | ASN | L | 158 | 16.789 | −23.122 | 33.757 | 1.00 | 18.77 | C |
| ATOM | 2913 | CG | ASN | L | 158 | 16.623 | −21.777 | 34.485 | 1.00 | 19.17 | C |
| ATOM | 2914 | OD1 | ASN | L | 158 | 15.607 | −21.552 | 35.161 | 1.00 | 20.23 | O |
| ATOM | 2915 | ND2 | ASN | L | 158 | 17.600 | −20.888 | 34.354 | 1.00 | 15.68 | N |
| ATOM | 2916 | C | ASN | L | 158 | 18.450 | −22.587 | 31.890 | 1.00 | 18.93 | C |
| ATOM | 2917 | O | ASN | L | 158 | 17.524 | −22.407 | 31.060 | 1.00 | 18.37 | O |
| ATOM | 2918 | N | SER | L | 159 | 19.699 | −22.166 | 31.699 | 1.00 | 18.61 | N |
| ATOM | 2919 | CA | SER | L | 159 | 20.095 | −21.481 | 30.471 | 1.00 | 18.78 | C |
| ATOM | 2920 | CB | SER | L | 159 | 20.241 | −19.974 | 30.701 | 1.00 | 18.78 | C |
| ATOM | 2921 | OG | SER | L | 159 | 21.193 | −19.687 | 31.710 | 1.00 | 19.12 | O |
| ATOM | 2922 | C | SER | L | 159 | 21.383 | −22.058 | 29.906 | 1.00 | 18.95 | C |
| ATOM | 2923 | O | SER | L | 159 | 22.216 | −22.584 | 30.645 | 1.00 | 18.08 | O |
| ATOM | 2924 | N | GLN | L | 160 | 21.534 | −21.977 | 28.585 | 1.00 | 18.95 | N |
| ATOM | 2925 | CA | GLN | L | 160 | 22.772 | −22.371 | 27.925 | 1.00 | 19.74 | C |
| ATOM | 2926 | CB | GLN | L | 160 | 22.605 | −23.731 | 27.217 | 1.00 | 20.32 | C |
| ATOM | 2927 | CG | GLN | L | 160 | 22.321 | −24.941 | 28.154 | 1.00 | 22.62 | C |
| ATOM | 2928 | CD | GLN | L | 160 | 22.292 | −26.298 | 27.428 | 1.00 | 24.89 | C |
| ATOM | 2929 | OE1 | GLN | L | 160 | 21.323 | −26.643 | 26.734 | 1.00 | 26.37 | O |
| ATOM | 2930 | NE2 | GLN | L | 160 | 23.353 | −27.074 | 27.597 | 1.00 | 24.24 | N |
| ATOM | 2931 | C | GLN | L | 160 | 23.170 | −21.273 | 26.918 | 1.00 | 19.85 | C |
| ATOM | 2932 | O | GLN | L | 160 | 22.304 | −20.667 | 26.282 | 1.00 | 19.01 | O |
| ATOM | 2933 | N | GLU | L | 161 | 24.477 | −21.021 | 26.795 | 1.00 | 19.52 | N |
| ATOM | 2934 | CA | GLU | L | 161 | 25.026 | −20.008 | 25.894 | 1.00 | 19.86 | C |
| ATOM | 2935 | CB | GLU | L | 161 | 26.041 | −19.120 | 26.620 | 1.00 | 20.21 | C |
| ATOM | 2936 | CG | GLU | L | 161 | 25.438 | −18.064 | 27.526 | 1.00 | 22.48 | C |
| ATOM | 2937 | CD | GLU | L | 161 | 26.492 | −17.151 | 28.078 | 1.00 | 23.73 | C |
| ATOM | 2938 | OE1 | GLU | L | 161 | 27.653 | −17.257 | 27.623 | 1.00 | 24.48 | O |
| ATOM | 2939 | OE2 | GLU | L | 161 | 26.169 | −16.329 | 28.962 | 1.00 | 24.27 | O |
| ATOM | 2940 | C | GLU | L | 161 | 25.761 | −20.672 | 24.730 | 1.00 | 19.95 | C |
| ATOM | 2941 | O | GLU | L | 161 | 26.335 | −21.760 | 24.882 | 1.00 | 19.39 | O |
| ATOM | 2942 | N | SER | L | 162 | 25.756 | −20.003 | 23.581 | 1.00 | 19.71 | N |
| ATOM | 2943 | CA | SER | L | 162 | 26.488 | −20.469 | 22.411 | 1.00 | 20.62 | C |
| ATOM | 2944 | CB | SER | L | 162 | 25.534 | −21.204 | 21.473 | 1.00 | 20.29 | C |
| ATOM | 2945 | OG | SER | L | 162 | 26.195 | −21.620 | 20.300 | 1.00 | 24.86 | O |
| ATOM | 2946 | C | SER | L | 162 | 27.124 | −19.246 | 21.736 | 1.00 | 20.18 | C |
| ATOM | 2947 | O | SER | L | 162 | 26.538 | −18.171 | 21.750 | 1.00 | 19.75 | O |
| ATOM | 2948 | N | VAL | L | 163 | 28.316 | −19.398 | 21.163 | 1.00 | 19.88 | N |
| ATOM | 2949 | CA | VAL | L | 163 | 29.030 | −18.232 | 20.622 | 1.00 | 20.58 | C |
| ATOM | 2950 | CB | VAL | L | 163 | 30.151 | −17.730 | 21.595 | 1.00 | 21.03 | C |
| ATOM | 2951 | CG1 | VAL | L | 163 | 31.280 | −18.757 | 21.723 | 1.00 | 23.18 | C |
| ATOM | 2952 | CG2 | VAL | L | 163 | 30.708 | −16.340 | 21.168 | 1.00 | 20.22 | C |
| ATOM | 2953 | C | VAL | L | 163 | 29.574 | −18.486 | 19.227 | 1.00 | 20.52 | C |
| ATOM | 2954 | O | VAL | L | 163 | 30.055 | −19.589 | 18.929 | 1.00 | 20.07 | O |
| ATOM | 2955 | N | THR | L | 164 | 29.486 | −17.472 | 18.364 | 1.00 | 19.95 | N |
| ATOM | 2956 | CA | THR | L | 164 | 30.020 | −17.621 | 17.025 | 1.00 | 20.15 | C |
| ATOM | 2957 | CB | THR | L | 164 | 29.439 | −16.591 | 16.038 | 1.00 | 20.24 | C |
| ATOM | 2958 | OG1 | THR | L | 164 | 29.684 | −15.264 | 16.523 | 1.00 | 20.91 | O |
| ATOM | 2959 | CG2 | THR | L | 164 | 27.976 | −16.793 | 15.863 | 1.00 | 19.22 | C |
| ATOM | 2960 | C | THR | L | 164 | 31.548 | −17.511 | 17.026 | 1.00 | 20.24 | C |
| ATOM | 2961 | O | THR | L | 164 | 32.160 | −16.988 | 17.970 | 1.00 | 19.70 | O |
| ATOM | 2962 | N | GLU | L | 165 | 32.154 | −18.030 | 15.968 | 1.00 | 20.46 | N |
| ATOM | 2963 | CA | GLU | L | 165 | 33.550 | −17.751 | 15.691 | 1.00 | 21.69 | C |
| ATOM | 2964 | CB | GLU | L | 165 | 34.067 | −18.657 | 14.562 | 1.00 | 22.31 | C |
| ATOM | 2965 | CG | GLU | L | 165 | 34.268 | −20.122 | 15.014 | 1.00 | 27.09 | C |
| ATOM | 2966 | CD | GLU | L | 165 | 35.079 | −20.216 | 16.306 | 1.00 | 32.01 | C |
| ATOM | 2967 | OE1 | GLU | L | 165 | 36.280 | −19.877 | 16.272 | 1.00 | 33.91 | O |
| ATOM | 2968 | OE2 | GLU | L | 165 | 34.505 | −20.585 | 17.371 | 1.00 | 36.56 | O |
| ATOM | 2969 | C | GLU | L | 165 | 33.643 | −16.279 | 15.298 | 1.00 | 20.88 | C |
| ATOM | 2970 | O | GLU | L | 165 | 32.677 | −15.714 | 14.802 | 1.00 | 19.71 | O |
| ATOM | 2971 | N | GLN | L | 166 | 34.805 | −15.671 | 15.530 | 1.00 | 20.89 | N |
| ATOM | 2972 | CA | GLN | L | 166 | 35.048 | −14.276 | 15.161 | 1.00 | 20.37 | C |
| ATOM | 2973 | CB | GLN | L | 166 | 36.498 | −13.887 | 15.486 | 1.00 | 20.21 | C |
| ATOM | 2974 | CG | GLN | L | 166 | 36.800 | −12.392 | 15.278 | 1.00 | 19.38 | C |
| ATOM | 2975 | CD | GLN | L | 166 | 38.153 | −11.950 | 15.848 | 1.00 | 17.74 | C |
| ATOM | 2976 | OE1 | GLN | L | 166 | 39.170 | −12.629 | 15.683 | 1.00 | 17.43 | O |
| ATOM | 2977 | NE2 | GLN | L | 166 | 38.162 | −10.796 | 16.519 | 1.00 | 15.88 | N |
| ATOM | 2978 | C | GLN | L | 166 | 34.758 | −14.053 | 13.688 | 1.00 | 20.56 | C |
| ATOM | 2979 | O | GLN | L | 166 | 35.232 | −14.805 | 12.844 | 1.00 | 20.33 | O |
| ATOM | 2980 | N | ASP | L | 167 | 33.971 | −13.030 | 13.378 | 1.00 | 20.97 | N |
| ATOM | 2981 | CA | ASP | L | 167 | 33.628 | −12.721 | 11.986 | 1.00 | 21.77 | C |
| ATOM | 2982 | CB | ASP | L | 167 | 32.594 | −11.620 | 11.918 | 1.00 | 21.51 | C |
| ATOM | 2983 | CG | ASP | L | 167 | 32.050 | −11.407 | 10.508 | 1.00 | 24.21 | C |
| ATOM | 2984 | OD1 | ASP | L | 167 | 32.649 | −10.630 | 9.730 | 1.00 | 23.90 | O |
| ATOM | 2985 | OD2 | ASP | L | 167 | 31.005 | −12.008 | 10.189 | 1.00 | 26.38 | O |
| ATOM | 2986 | C | ASP | L | 167 | 34.869 | −12.315 | 11.193 | 1.00 | 22.27 | C |
| ATOM | 2987 | O | ASP | L | 167 | 35.619 | −11.421 | 11.597 | 1.00 | 22.02 | O |
| ATOM | 2988 | N | SER | L | 168 | 35.072 | −12.961 | 10.052 | 1.00 | 22.74 | N |
| ATOM | 2989 | CA | SER | L | 168 | 36.294 | −12.752 | 9.283 | 1.00 | 23.95 | C |
| ATOM | 2990 | CB | SER | L | 168 | 36.389 | −13.777 | 8.159 | 1.00 | 23.89 | C |
| ATOM | 2991 | OG | SER | L | 168 | 35.422 | −13.480 | 7.164 | 1.00 | 26.88 | O |
| ATOM | 2992 | C | SER | L | 168 | 36.366 | −11.322 | 8.728 | 1.00 | 24.22 | C |
| ATOM | 2993 | O | SER | L | 168 | 37.451 | −10.807 | 8.481 | 1.00 | 24.98 | O |
| ATOM | 2994 | N | LYS | L | 169 | 35.218 | −10.673 | 8.558 | 1.00 | 23.91 | N |
| ATOM | 2995 | CA | LYS | L | 169 | 35.214 | −9.337 | 7.981 | 1.00 | 24.12 | C |
| ATOM | 2996 | CB | LYS | L | 169 | 34.099 | −9.200 | 6.947 | 1.00 | 24.81 | C |
| ATOM | 2997 | CG | LYS | L | 169 | 34.589 | −8.627 | 5.627 | 1.00 | 27.44 | C |
| ATOM | 2998 | CD | LYS | L | 169 | 33.457 | −7.994 | 4.827 | 1.00 | 30.40 | C |
| ATOM | 2999 | CE | LYS | L | 169 | 32.764 | −6.871 | 5.611 | 1.00 | 32.19 | C |
| ATOM | 3000 | NZ | LYS | L | 169 | 31.642 | −6.282 | 4.833 | 1.00 | 33.44 | N |
| ATOM | 3001 | C | LYS | L | 169 | 35.159 | −8.195 | 9.007 | 1.00 | 23.30 | C |
| ATOM | 3002 | O | LYS | L | 169 | 35.894 | −7.218 | 8.872 | 1.00 | 23.59 | O |
| ATOM | 3003 | N | ASP | L | 170 | 34.306 | −8.300 | 10.023 | 1.00 | 21.82 | N |
| ATOM | 3004 | CA | ASP | L | 170 | 34.221 | −7.223 | 11.022 | 1.00 | 20.80 | C |
| ATOM | 3005 | CB | ASP | L | 170 | 32.775 | −6.703 | 11.177 | 1.00 | 21.02 | C |
| ATOM | 3006 | CG | ASP | L | 170 | 31.858 | −7.665 | 11.931 | 1.00 | 21.94 | C |
| ATOM | 3007 | OD1 | ASP | L | 170 | 32.349 | −8.572 | 12.631 | 1.00 | 22.02 | O |
| ATOM | 3008 | OD2 | ASP | L | 170 | 30.620 | −7.486 | 11.849 | 1.00 | 23.95 | O |
| ATOM | 3009 | C | ASP | L | 170 | 34.873 | −7.546 | 12.379 | 1.00 | 19.90 | C |
| ATOM | 3010 | O | ASP | L | 170 | 34.867 | −6.724 | 13.292 | 1.00 | 19.52 | O |
| ATOM | 3011 | N | SER | L | 171 | 35.422 | −8.754 | 12.498 | 1.00 | 18.98 | N |
| ATOM | 3012 | CA | SER | L | 171 | 36.124 | −9.188 | 13.707 | 1.00 | 18.66 | C |
| ATOM | 3013 | CB | SER | L | 171 | 37.402 | −8.361 | 13.903 | 1.00 | 18.25 | C |
| ATOM | 3014 | OG | SER | L | 171 | 38.253 | −8.511 | 12.780 | 1.00 | 19.69 | O |
| ATOM | 3015 | C | SER | L | 171 | 35.260 | −9.159 | 14.986 | 1.00 | 18.18 | C |
| ATOM | 3016 | O | SER | L | 171 | 35.800 | −9.026 | 16.088 | 1.00 | 18.16 | O |
| ATOM | 3017 | N | THR | L | 172 | 33.940 | −9.291 | 14.842 | 1.00 | 18.15 | N |
| ATOM | 3018 | CA | THR | L | 172 | 33.062 | −9.332 | 16.015 | 1.00 | 18.24 | C |
| ATOM | 3019 | CB | THR | L | 172 | 31.823 | −8.416 | 15.896 | 1.00 | 18.81 | C |
| ATOM | 3020 | OG1 | THR | L | 172 | 30.992 | −8.843 | 14.811 | 1.00 | 19.87 | O |
| ATOM | 3021 | CG2 | THR | L | 172 | 32.222 | −6.952 | 15.721 | 1.00 | 18.95 | C |
| ATOM | 3022 | C | THR | L | 172 | 32.596 | −10.743 | 16.333 | 1.00 | 18.19 | C |
| ATOM | 3023 | O | THR | L | 172 | 32.751 | −11.658 | 15.512 | 1.00 | 17.85 | O |
| ATOM | 3024 | N | TYR | L | 173 | 32.038 | −10.904 | 17.531 | 1.00 | 17.84 | N |
| ATOM | 3025 | CA | TYR | L | 173 | 31.372 | −12.133 | 17.935 | 1.00 | 17.65 | C |
| ATOM | 3026 | CB | TYR | L | 173 | 31.924 | −12.620 | 19.278 | 1.00 | 18.30 | C |
| ATOM | 3027 | CG | TYR | L | 173 | 33.394 | −12.970 | 19.237 | 1.00 | 19.02 | C |
| ATOM | 3028 | CD1 | TYR | L | 173 | 34.366 | −12.018 | 19.553 | 1.00 | 21.01 | C |
| ATOM | 3029 | CE1 | TYR | L | 173 | 35.725 | −12.336 | 19.519 | 1.00 | 23.47 | C |
| ATOM | 3030 | CZ | TYR | L | 173 | 36.127 | −13.615 | 19.148 | 1.00 | 23.76 | C |
| ATOM | 3031 | OH | TYR | L | 173 | 37.463 | −13.924 | 19.095 | 1.00 | 25.21 | O |
| ATOM | 3032 | CE2 | TYR | L | 173 | 35.191 | −14.577 | 18.814 | 1.00 | 23.22 | C |
| ATOM | 3033 | CD2 | TYR | L | 173 | 33.817 | −14.248 | 18.856 | 1.00 | 20.55 | C |
| ATOM | 3034 | C | TYR | L | 173 | 29.861 | −11.900 | 18.046 | 1.00 | 17.91 | C |
| ATOM | 3035 | O | TYR | L | 173 | 29.388 | −10.754 | 18.138 | 1.00 | 17.86 | O |
| ATOM | 3036 | N | SER | L | 174 | 29.099 | −12.986 | 18.018 | 1.00 | 17.45 | N |
| ATOM | 3037 | CA | SER | L | 174 | 27.711 | −12.930 | 18.441 | 1.00 | 16.91 | C |
| ATOM | 3038 | CB | SER | L | 174 | 26.777 | −12.954 | 17.235 | 1.00 | 16.74 | C |
| ATOM | 3039 | OG | SER | L | 174 | 26.941 | −11.770 | 16.475 | 1.00 | 17.19 | O |
| ATOM | 3040 | C | SER | L | 174 | 27.437 | −14.075 | 19.412 | 1.00 | 16.78 | C |
| ATOM | 3041 | O | SER | L | 174 | 28.126 | −15.091 | 19.389 | 1.00 | 16.32 | O |
| ATOM | 3042 | N | LEU | L | 175 | 26.451 | −13.897 | 20.283 | 1.00 | 16.59 | N |
| ATOM | 3043 | CA | LEU | L | 175 | 26.201 | −14.860 | 21.349 | 1.00 | 16.72 | C |
| ATOM | 3044 | CB | LEU | L | 175 | 26.882 | −14.393 | 22.643 | 1.00 | 17.17 | C |
| ATOM | 3045 | CG | LEU | L | 175 | 26.938 | −15.258 | 23.917 | 1.00 | 18.09 | C |
| ATOM | 3046 | CD1 | LEU | L | 175 | 28.068 | −14.747 | 24.848 | 1.00 | 20.19 | C |
| ATOM | 3047 | CD2 | LEU | L | 175 | 25.633 | −15.265 | 24.661 | 1.00 | 19.25 | C |
| ATOM | 3048 | C | LEU | L | 175 | 24.705 | −15.047 | 21.553 | 1.00 | 16.61 | C |
| ATOM | 3049 | O | LEU | L | 175 | 23.948 | −14.092 | 21.488 | 1.00 | 16.32 | O |
| ATOM | 3050 | N | SER | L | 176 | 24.280 | −16.284 | 21.797 | 1.00 | 17.08 | N |
| ATOM | 3051 | CA | SER | L | 176 | 22.894 | −16.554 | 22.140 | 1.00 | 17.97 | C |
| ATOM | 3052 | CB | SER | L | 176 | 22.241 | −17.517 | 21.145 | 1.00 | 18.69 | C |
| ATOM | 3053 | OG | SER | L | 176 | 22.836 | −18.811 | 21.237 | 1.00 | 22.25 | O |
| ATOM | 3054 | C | SER | L | 176 | 22.854 | −17.149 | 23.530 | 1.00 | 18.00 | C |
| ATOM | 3055 | O | SER | L | 176 | 23.720 | −17.950 | 23.898 | 1.00 | 18.80 | O |
| ATOM | 3056 | N | SER | L | 177 | 21.875 | −16.735 | 24.317 | 1.00 | 17.21 | N |
| ATOM | 3057 | CA | SER | L | 177 | 21.615 | −17.396 | 25.585 | 1.00 | 17.26 | C |
| ATOM | 3058 | CB | SER | L | 177 | 21.866 | −16.459 | 26.764 | 1.00 | 17.01 | C |
| ATOM | 3059 | OG | SER | L | 177 | 21.552 | −17.116 | 27.980 | 1.00 | 17.47 | O |
| ATOM | 3060 | C | SER | L | 177 | 20.179 | −17.854 | 25.568 | 1.00 | 16.58 | C |
| ATOM | 3061 | O | SER | L | 177 | 19.288 | −17.073 | 25.250 | 1.00 | 17.29 | O |
| ATOM | 3062 | N | THR | L | 178 | 19.955 | −19.127 | 25.870 | 1.00 | 16.08 | N |
| ATOM | 3063 | CA | THR | L | 178 | 18.608 | −19.689 | 25.800 | 1.00 | 15.78 | C |
| ATOM | 3064 | CB | THR | L | 178 | 18.509 | −20.836 | 24.771 | 1.00 | 16.06 | C |
| ATOM | 3065 | OG1 | THR | L | 178 | 18.947 | −20.364 | 23.498 | 1.00 | 18.43 | O |
| ATOM | 3066 | CG2 | THR | L | 178 | 17.069 | −21.331 | 24.648 | 1.00 | 16.09 | C |
| ATOM | 3067 | C | THR | L | 178 | 18.146 | −20.178 | 27.162 | 1.00 | 14.98 | C |
| ATOM | 3068 | O | THR | L | 178 | 18.761 | −21.079 | 27.760 | 1.00 | 14.04 | O |
| ATOM | 3069 | N | LEU | L | 179 | 17.073 | −19.556 | 27.650 | 1.00 | 14.85 | N |
| ATOM | 3070 | CA | LEU | L | 179 | 16.434 | −19.943 | 28.893 | 1.00 | 14.51 | C |
| ATOM | 3071 | CB | LEU | L | 179 | 15.828 | −18.716 | 29.587 | 1.00 | 14.21 | C |
| ATOM | 3072 | CG | LEU | L | 179 | 15.004 | −18.933 | 30.865 | 1.00 | 14.21 | C |
| ATOM | 3073 | CD1 | LEU | L | 179 | 15.918 | −19.405 | 31.985 | 1.00 | 14.66 | C |
| ATOM | 3074 | CD2 | LEU | L | 179 | 14.268 | −17.655 | 31.310 | 1.00 | 12.75 | C |
| ATOM | 3075 | C | LEU | L | 179 | 15.353 | −20.968 | 28.583 | 1.00 | 15.26 | C |
| ATOM | 3076 | O | LEU | L | 179 | 14.460 | −20.714 | 27.765 | 1.00 | 15.19 | O |
| ATOM | 3077 | N | THR | L | 180 | 15.423 | −22.122 | 29.236 | 1.00 | 15.90 | N |
| ATOM | 3078 | CA | THR | L | 180 | 14.454 | −23.201 | 28.999 | 1.00 | 16.68 | C |
| ATOM | 3079 | CB | THR | L | 180 | 15.149 | −24.488 | 28.498 | 1.00 | 16.92 | C |
| ATOM | 3080 | OG1 | THR | L | 180 | 15.811 | −24.200 | 27.267 | 1.00 | 16.63 | O |
| ATOM | 3081 | CG2 | THR | L | 180 | 14.129 | −25.602 | 28.256 | 1.00 | 17.60 | C |
| ATOM | 3082 | C | THR | L | 180 | 13.617 | −23.487 | 30.237 | 1.00 | 17.28 | C |
| ATOM | 3083 | O | THR | L | 180 | 14.156 | −23.706 | 31.322 | 1.00 | 16.65 | O |
| ATOM | 3084 | N | LEU | L | 181 | 12.292 | −23.456 | 30.052 | 1.00 | 17.36 | N |
| ATOM | 3085 | CA | LEU | L | 181 | 11.330 | −23.702 | 31.119 | 1.00 | 18.84 | C |
| ATOM | 3086 | CB | LEU | L | 181 | 10.686 | −22.381 | 31.570 | 1.00 | 18.74 | C |
| ATOM | 3087 | CG | LEU | L | 181 | 11.591 | −21.254 | 32.090 | 1.00 | 19.80 | C |
| ATOM | 3088 | CD1 | LEU | L | 181 | 10.784 | −20.071 | 32.589 | 1.00 | 22.06 | C |
| ATOM | 3089 | CD2 | LEU | L | 181 | 12.497 | −21.738 | 33.201 | 1.00 | 21.14 | C |
| ATOM | 3090 | C | LEU | L | 181 | 10.236 | −24.651 | 30.644 | 1.00 | 19.36 | C |
| ATOM | 3091 | O | LEU | L | 181 | 9.964 | −24.745 | 29.439 | 1.00 | 19.31 | O |
| ATOM | 3092 | N | SER | L | 182 | 9.587 | −25.346 | 31.578 | 1.00 | 20.02 | N |
| ATOM | 3093 | CA | SER | L | 182 | 8.371 | −26.059 | 31.211 | 1.00 | 20.47 | C |
| ATOM | 3094 | CB | SER | L | 182 | 7.924 | −27.046 | 32.299 | 1.00 | 20.65 | C |
| ATOM | 3095 | OG | SER | L | 182 | 7.719 | −26.390 | 33.524 | 1.00 | 21.13 | O |
| ATOM | 3096 | C | SER | L | 182 | 7.304 | −24.999 | 30.949 | 1.00 | 20.74 | C |
| ATOM | 3097 | O | SER | L | 182 | 7.431 | −23.859 | 31.411 | 1.00 | 20.31 | O |
| ATOM | 3098 | N | LYS | L | 183 | 6.280 | −25.365 | 30.179 | 1.00 | 21.18 | N |
| ATOM | 3099 | CA | LYS | L | 183 | 5.155 | −24.477 | 29.920 | 1.00 | 21.76 | C |
| ATOM | 3100 | CB | LYS | L | 183 | 4.163 | −25.159 | 28.971 | 1.00 | 21.95 | C |
| ATOM | 3101 | CG | LYS | L | 183 | 2.890 | −24.364 | 28.645 | 1.00 | 24.09 | C |
| ATOM | 3102 | CD | LYS | L | 183 | 1.737 | −25.294 | 28.221 | 1.00 | 25.49 | C |
| ATOM | 3103 | CE | LYS | L | 183 | 0.447 | −24.503 | 27.976 | 1.00 | 26.24 | C |
| ATOM | 3104 | NZ | LYS | L | 183 | −0.809 | −25.323 | 28.027 | 1.00 | 27.31 | N |
| ATOM | 3105 | C | LYS | L | 183 | 4.498 | −24.091 | 31.260 | 1.00 | 22.01 | C |
| ATOM | 3106 | O | LYS | L | 183 | 4.229 | −22.910 | 31.505 | 1.00 | 21.50 | O |
| ATOM | 3107 | N | ALA | L | 184 | 4.297 | −25.085 | 32.128 | 1.00 | 22.43 | N |
| ATOM | 3108 | CA | ALA | L | 184 | 3.753 | −24.861 | 33.480 | 1.00 | 23.72 | C |
| ATOM | 3109 | CB | ALA | L | 184 | 3.657 | −26.181 | 34.265 | 1.00 | 23.61 | C |
| ATOM | 3110 | C | ALA | L | 184 | 4.551 | −23.818 | 34.264 | 1.00 | 23.87 | C |
| ATOM | 3111 | O | ALA | L | 184 | 3.970 | −22.947 | 34.903 | 1.00 | 24.19 | O |
| ATOM | 3112 | N | ASP | L | 185 | 5.877 | −23.896 | 34.188 | 1.00 | 24.46 | N |
| ATOM | 3113 | CA | ASP | L | 185 | 6.736 | −22.936 | 34.873 | 1.00 | 24.55 | C |
| ATOM | 3114 | CB | ASP | L | 185 | 8.146 | −23.508 | 35.050 | 1.00 | 25.07 | C |
| ATOM | 3115 | CG | ASP | L | 185 | 8.208 | −24.596 | 36.121 | 1.00 | 26.02 | C |
| ATOM | 3116 | OD1 | ASP | L | 185 | 9.269 | −25.233 | 36.271 | 1.00 | 27.90 | O |
| ATOM | 3117 | OD2 | ASP | L | 185 | 7.199 | −24.822 | 36.814 | 1.00 | 27.75 | O |
| ATOM | 3118 | C | ASP | L | 185 | 6.766 | −21.560 | 34.204 | 1.00 | 24.23 | C |
| ATOM | 3119 | O | ASP | L | 185 | 6.834 | −20.545 | 34.891 | 1.00 | 24.18 | O |
| ATOM | 3120 | N | TYR | L | 186 | 6.707 | −21.527 | 32.872 | 1.00 | 23.86 | N |
| ATOM | 3121 | CA | TYR | L | 186 | 6.686 | −20.262 | 32.120 | 1.00 | 23.34 | C |
| ATOM | 3122 | CB | TYR | L | 186 | 6.824 | −20.529 | 30.619 | 1.00 | 22.79 | C |
| ATOM | 3123 | CG | TYR | L | 186 | 6.662 | −19.318 | 29.724 | 1.00 | 21.39 | C |
| ATOM | 3124 | CD1 | TYR | L | 186 | 7.640 | −18.324 | 29.670 | 1.00 | 21.13 | C |
| ATOM | 3125 | CE1 | TYR | L | 186 | 7.503 | −17.218 | 28.831 | 1.00 | 20.98 | C |
| ATOM | 3126 | CZ | TYR | L | 186 | 6.378 | −17.107 | 28.022 | 1.00 | 21.55 | C |
| ATOM | 3127 | OH | TYR | L | 186 | 6.237 | −16.019 | 27.188 | 1.00 | 21.79 | O |
| ATOM | 3128 | CE2 | TYR | L | 186 | 5.398 | −18.088 | 28.045 | 1.00 | 20.41 | C |
| ATOM | 3129 | CD2 | TYR | L | 186 | 5.543 | −19.185 | 28.893 | 1.00 | 21.02 | C |
| ATOM | 3130 | C | TYR | L | 186 | 5.457 | −19.377 | 32.391 | 1.00 | 23.95 | C |
| ATOM | 3131 | O | TYR | L | 186 | 5.581 | −18.152 | 32.515 | 1.00 | 24.04 | O |
| ATOM | 3132 | N | GLU | L | 187 | 4.276 | −19.988 | 32.465 | 1.00 | 24.38 | N |
| ATOM | 3133 | CA | GLU | L | 187 | 3.042 | −19.228 | 32.704 | 1.00 | 24.43 | C |
| ATOM | 3134 | CB | GLU | L | 187 | 1.811 | −19.970 | 32.150 | 1.00 | 24.39 | C |
| ATOM | 3135 | CG | GLU | L | 187 | 1.788 | −20.176 | 30.592 | 1.00 | 25.14 | C |
| ATOM | 3136 | CD | GLU | L | 187 | 1.439 | −18.914 | 29.773 | 1.00 | 24.43 | C |
| ATOM | 3137 | OE1 | GLU | L | 187 | 0.828 | −17.962 | 30.314 | 1.00 | 23.64 | O |
| ATOM | 3138 | OE2 | GLU | L | 187 | 1.777 | −18.881 | 28.569 | 1.00 | 25.58 | O |
| ATOM | 3139 | C | GLU | L | 187 | 2.839 | −18.812 | 34.177 | 1.00 | 24.64 | C |
| ATOM | 3140 | O | GLU | L | 187 | 1.898 | −18.063 | 34.484 | 1.00 | 24.48 | O |
| ATOM | 3141 | N | LYS | L | 188 | 3.709 | −19.290 | 35.073 | 1.00 | 24.81 | N |
| ATOM | 3142 | CA | LYS | L | 188 | 3.709 | −18.850 | 36.484 | 1.00 | 25.09 | C |
| ATOM | 3143 | CB | LYS | L | 188 | 4.642 | −19.708 | 37.359 | 1.00 | 25.29 | C |
| ATOM | 3144 | CG | LYS | L | 188 | 4.119 | −21.089 | 37.727 | 1.00 | 25.42 | C |
| ATOM | 3145 | CD | LYS | L | 188 | 2.906 | −21.003 | 38.645 | 1.00 | 27.62 | C |
| ATOM | 3146 | CE | LYS | L | 188 | 1.916 | −22.134 | 38.376 | 1.00 | 27.33 | C |
| ATOM | 3147 | NZ | LYS | L | 188 | 0.616 | −21.908 | 39.079 | 1.00 | 29.18 | N |
| ATOM | 3148 | C | LYS | L | 188 | 4.119 | −17.384 | 36.631 | 1.00 | 25.22 | C |
| ATOM | 3149 | O | LYS | L | 188 | 3.663 | −16.697 | 37.545 | 1.00 | 25.32 | O |
| ATOM | 3150 | N | HIS | L | 189 | 4.982 | −16.915 | 35.732 | 1.00 | 24.78 | N |
| ATOM | 3151 | CA | HIS | L | 189 | 5.587 | −15.590 | 35.856 | 1.00 | 24.58 | C |
| ATOM | 3152 | CB | HIS | L | 189 | 7.095 | −15.738 | 36.041 | 1.00 | 24.69 | C |
| ATOM | 3153 | CG | HIS | L | 189 | 7.466 | −16.558 | 37.233 | 1.00 | 25.64 | C |
| ATOM | 3154 | ND1 | HIS | L | 189 | 7.256 | −16.126 | 38.527 | 1.00 | 25.78 | N |
| ATOM | 3155 | CE1 | HIS | L | 189 | 7.667 | −17.055 | 39.372 | 1.00 | 26.57 | C |
| ATOM | 3156 | NE2 | HIS | L | 189 | 8.134 | −18.076 | 38.672 | 1.00 | 27.06 | N |
| ATOM | 3157 | CD2 | HIS | L | 189 | 8.020 | −17.791 | 37.332 | 1.00 | 27.42 | C |
| ATOM | 3158 | C | HIS | L | 189 | 5.264 | −14.648 | 34.694 | 1.00 | 24.25 | C |
| ATOM | 3159 | O | HIS | L | 189 | 4.800 | −15.080 | 33.634 | 1.00 | 24.23 | O |
| ATOM | 3160 | N | LYS | L | 190 | 5.523 | −13.359 | 34.906 | 1.00 | 23.95 | N |
| ATOM | 3161 | CA | LYS | L | 190 | 5.157 | −12.310 | 33.953 | 1.00 | 23.43 | C |
| ATOM | 3162 | CB | LYS | L | 190 | 4.388 | −11.205 | 34.689 | 1.00 | 23.96 | C |
| ATOM | 3163 | CG | LYS | L | 190 | 3.852 | −10.086 | 33.798 | 1.00 | 25.07 | C |
| ATOM | 3164 | CD | LYS | L | 190 | 3.168 | −8.991 | 34.620 | 1.00 | 26.14 | C |
| ATOM | 3165 | CE | LYS | L | 190 | 2.574 | −7.918 | 33.706 | 1.00 | 27.92 | C |
| ATOM | 3166 | NZ | LYS | L | 190 | 2.134 | −6.703 | 34.460 | 1.00 | 28.56 | N |
| ATOM | 3167 | C | LYS | L | 190 | 6.373 | −11.716 | 33.242 | 1.00 | 22.96 | C |
| ATOM | 3168 | O | LYS | L | 190 | 6.470 | −11.748 | 32.007 | 1.00 | 22.07 | O |
| ATOM | 3169 | N | VAL | L | 191 | 7.291 | −11.171 | 34.033 | 1.00 | 21.94 | N |
| ATOM | 3170 | CA | VAL | L | 191 | 8.416 | −10.422 | 33.501 | 1.00 | 21.94 | C |
| ATOM | 3171 | CB | VAL | L | 191 | 8.858 | −9.281 | 34.462 | 1.00 | 22.06 | C |
| ATOM | 3172 | CG1 | VAL | L | 191 | 10.003 | −8.467 | 33.843 | 1.00 | 21.89 | C |
| ATOM | 3173 | CG2 | VAL | L | 191 | 7.657 | −8.374 | 34.833 | 1.00 | 21.67 | C |
| ATOM | 3174 | C | VAL | L | 191 | 9.594 | −11.357 | 33.209 | 1.00 | 21.63 | C |
| ATOM | 3175 | O | VAL | L | 191 | 10.110 | −12.027 | 34.115 | 1.00 | 21.34 | O |
| ATOM | 3176 | N | TYR | L | 192 | 9.988 | −11.398 | 31.937 | 1.00 | 21.30 | N |
| ATOM | 3177 | CA | TYR | L | 192 | 11.174 | −12.130 | 31.483 | 1.00 | 21.05 | C |
| ATOM | 3178 | CB | TYR | L | 192 | 10.787 | −13.164 | 30.424 | 1.00 | 20.97 | C |
| ATOM | 3179 | CG | TYR | L | 192 | 10.021 | −14.306 | 31.032 | 1.00 | 21.16 | C |
| ATOM | 3180 | CD1 | TYR | L | 192 | 8.647 | −14.233 | 31.211 | 1.00 | 20.50 | C |
| ATOM | 3181 | CE1 | TYR | L | 192 | 7.951 | −15.280 | 31.795 | 1.00 | 21.80 | C |
| ATOM | 3182 | CZ | TYR | L | 192 | 8.642 | −16.405 | 32.203 | 1.00 | 21.75 | C |
| ATOM | 3183 | OH | TYR | L | 192 | 7.996 | −17.466 | 32.778 | 1.00 | 22.36 | O |
| ATOM | 3184 | CE2 | TYR | L | 192 | 10.000 | −16.490 | 32.036 | 1.00 | 21.98 | C |
| ATOM | 3185 | CD2 | TYR | L | 192 | 10.680 | −15.446 | 31.458 | 1.00 | 21.92 | C |
| ATOM | 3186 | C | TYR | L | 192 | 12.202 | −11.157 | 30.933 | 1.00 | 21.11 | C |
| ATOM | 3187 | O | TYR | L | 192 | 11.922 | −10.417 | 29.992 | 1.00 | 20.82 | O |
| ATOM | 3188 | N | ALA | L | 193 | 13.390 | −11.172 | 31.530 | 1.00 | 20.96 | N |
| ATOM | 3189 | CA | ALA | L | 193 | 14.396 | −10.140 | 31.300 | 1.00 | 21.06 | C |
| ATOM | 3190 | CB | ALA | L | 193 | 14.457 | −9.178 | 32.506 | 1.00 | 20.99 | C |
| ATOM | 3191 | C | ALA | L | 193 | 15.760 | −10.750 | 31.036 | 1.00 | 21.28 | C |
| ATOM | 3192 | O | ALA | L | 193 | 16.152 | −11.717 | 31.700 | 1.00 | 21.12 | O |
| ATOM | 3193 | N | CYS | L | 194 | 16.462 | −10.191 | 30.049 | 1.00 | 21.25 | N |
| ATOM | 3194 | CA | CYS | L | 194 | 17.834 | −10.558 | 29.747 | 1.00 | 22.17 | C |
| ATOM | 3195 | CB | CYS | L | 194 | 17.978 | −10.915 | 28.258 | 1.00 | 22.23 | C |
| ATOM | 3196 | SG | CYS | L | 194 | 19.631 | −11.379 | 27.876 | 1.00 | 27.90 | S |
| ATOM | 3197 | C | CYS | L | 194 | 18.736 | −9.373 | 30.078 | 1.00 | 21.50 | C |
| ATOM | 3198 | O | CYS | L | 194 | 18.603 | −8.318 | 29.453 | 1.00 | 21.58 | O |
| ATOM | 3199 | N | GLU | L | 195 | 19.642 | −9.542 | 31.050 | 1.00 | 20.87 | N |
| ATOM | 3200 | CA | GLU | L | 195 | 20.561 | −8.470 | 31.446 | 1.00 | 20.27 | C |
| ATOM | 3201 | CB | GLU | L | 195 | 20.513 | −8.232 | 32.961 | 1.00 | 20.87 | C |
| ATOM | 3202 | CG | GLU | L | 195 | 21.370 | −7.057 | 33.438 | 1.00 | 22.88 | C |
| ATOM | 3203 | CD | GLU | L | 195 | 21.578 | −7.035 | 34.953 | 1.00 | 25.61 | C |
| ATOM | 3204 | OE1 | GLU | L | 195 | 22.387 | −7.847 | 35.457 | 1.00 | 27.83 | O |
| ATOM | 3205 | OE2 | GLU | L | 195 | 20.947 | −6.202 | 35.636 | 1.00 | 27.03 | O |
| ATOM | 3206 | C | GLU | L | 195 | 22.003 | −8.725 | 30.992 | 1.00 | 19.63 | C |
| ATOM | 3207 | O | GLU | L | 195 | 22.617 | −9.724 | 31.378 | 1.00 | 18.89 | O |
| ATOM | 3208 | N | VAL | L | 196 | 22.540 | −7.790 | 30.212 | 1.00 | 19.01 | N |
| ATOM | 3209 | CA | VAL | L | 196 | 23.832 | −7.968 | 29.530 | 1.00 | 19.10 | C |
| ATOM | 3210 | CB | VAL | L | 196 | 23.701 | −7.770 | 27.997 | 1.00 | 18.98 | C |
| ATOM | 3211 | CG1 | VAL | L | 196 | 25.075 | −7.840 | 27.301 | 1.00 | 19.60 | C |
| ATOM | 3212 | CG2 | VAL | L | 196 | 22.750 | −8.791 | 27.401 | 1.00 | 18.72 | C |
| ATOM | 3213 | C | VAL | L | 196 | 24.877 | −6.992 | 30.044 | 1.00 | 19.13 | C |
| ATOM | 3214 | O | VAL | L | 196 | 24.641 | −5.782 | 30.073 | 1.00 | 18.65 | O |
| ATOM | 3215 | N | THR | L | 197 | 26.030 | −7.534 | 30.421 | 1.00 | 19.13 | N |
| ATOM | 3216 | CA | THR | L | 197 | 27.164 | −6.759 | 30.893 | 1.00 | 20.17 | C |
| ATOM | 3217 | CB | THR | L | 197 | 27.572 | −7.218 | 32.326 | 1.00 | 20.00 | C |
| ATOM | 3218 | OG1 | THR | L | 197 | 26.527 | −6.882 | 33.255 | 1.00 | 20.98 | O |
| ATOM | 3219 | CG2 | THR | L | 197 | 28.862 | −6.576 | 32.783 | 1.00 | 21.50 | C |
| ATOM | 3220 | C | THR | L | 197 | 28.295 | −6.931 | 29.877 | 1.00 | 20.46 | C |
| ATOM | 3221 | O | THR | L | 197 | 28.611 | −8.054 | 29.478 | 1.00 | 20.92 | O |
| ATOM | 3222 | N | HIS | L | 198 | 28.864 | −5.814 | 29.426 | 1.00 | 20.90 | N |
| ATOM | 3223 | CA | HIS | L | 198 | 29.934 | −5.829 | 28.423 | 1.00 | 21.62 | C |
| ATOM | 3224 | CB | HIS | L | 198 | 29.372 | −6.026 | 26.994 | 1.00 | 21.25 | C |
| ATOM | 3225 | CG | HIS | L | 198 | 30.432 | −6.209 | 25.951 | 1.00 | 20.24 | C |
| ATOM | 3226 | ND1 | HIS | L | 198 | 30.970 | −5.156 | 25.243 | 1.00 | 19.84 | N |
| ATOM | 3227 | CE1 | HIS | L | 198 | 31.901 | −5.608 | 24.419 | 1.00 | 19.09 | C |
| ATOM | 3228 | NE2 | HIS | L | 198 | 31.990 | −6.917 | 24.569 | 1.00 | 18.43 | N |
| ATOM | 3229 | CD2 | HIS | L | 198 | 31.081 | −7.319 | 25.520 | 1.00 | 19.76 | C |
| ATOM | 3230 | C | HIS | L | 198 | 30.750 | −4.540 | 28.492 | 1.00 | 22.28 | C |
| ATOM | 3231 | O | HIS | L | 198 | 30.204 | −3.472 | 28.797 | 1.00 | 22.41 | O |
| ATOM | 3232 | N | GLN | L | 199 | 32.044 | −4.656 | 28.196 | 1.00 | 23.00 | N |
| ATOM | 3233 | CA | GLN | L | 199 | 32.986 | −3.528 | 28.212 | 1.00 | 23.99 | C |
| ATOM | 3234 | CB | GLN | L | 199 | 34.389 | −4.002 | 27.778 | 1.00 | 24.29 | C |
| ATOM | 3235 | CG | GLN | L | 199 | 35.354 | −2.867 | 27.454 | 1.00 | 26.47 | C |
| ATOM | 3236 | CD | GLN | L | 199 | 36.769 | −3.325 | 27.159 | 1.00 | 28.83 | C |
| ATOM | 3237 | OE1 | GLN | L | 199 | 37.080 | −4.512 | 27.193 | 1.00 | 29.87 | O |
| ATOM | 3238 | NE2 | GLN | L | 199 | 37.641 | −2.366 | 26.874 | 1.00 | 30.37 | N |
| ATOM | 3239 | C | GLN | L | 199 | 32.519 | −2.344 | 27.355 | 1.00 | 23.85 | C |
| ATOM | 3240 | O | GLN | L | 199 | 32.786 | −1.193 | 27.688 | 1.00 | 24.26 | O |
| ATOM | 3241 | N | GLY | L | 200 | 31.817 | −2.635 | 26.263 | 1.00 | 23.74 | N |
| ATOM | 3242 | CA | GLY | L | 200 | 31.271 | −1.609 | 25.385 | 1.00 | 24.20 | C |
| ATOM | 3243 | C | GLY | L | 200 | 30.099 | −0.804 | 25.927 | 1.00 | 24.61 | C |
| ATOM | 3244 | O | GLY | L | 200 | 29.791 | 0.251 | 25.383 | 1.00 | 24.97 | O |
| ATOM | 3245 | N | LEU | L | 201 | 29.449 | −1.282 | 26.989 | 1.00 | 25.03 | N |
| ATOM | 3246 | CA | LEU | L | 201 | 28.268 | −0.585 | 27.561 | 1.00 | 26.21 | C |
| ATOM | 3247 | CB | LEU | L | 201 | 27.130 | −1.580 | 27.807 | 1.00 | 25.68 | C |
| ATOM | 3248 | CG | LEU | L | 201 | 26.658 | −2.459 | 26.645 | 1.00 | 25.06 | C |
| ATOM | 3249 | CD1 | LEU | L | 201 | 26.012 | −3.742 | 27.157 | 1.00 | 23.92 | C |
| ATOM | 3250 | CD2 | LEU | L | 201 | 25.692 | −1.687 | 25.772 | 1.00 | 24.73 | C |
| ATOM | 3251 | C | LEU | L | 201 | 28.597 | 0.148 | 28.864 | 1.00 | 26.74 | C |
| ATOM | 3252 | O | LEU | L | 201 | 29.231 | −0.427 | 29.748 | 1.00 | 27.09 | O |
| ATOM | 3253 | N | SER | L | 202 | 28.163 | 1.409 | 28.970 | 1.00 | 28.09 | N |
| ATOM | 3254 | CA | SER | L | 202 | 28.344 | 2.242 | 30.180 | 1.00 | 28.98 | C |
| ATOM | 3255 | CB | SER | L | 202 | 27.536 | 3.545 | 30.084 | 1.00 | 29.11 | C |
| ATOM | 3256 | OG | SER | L | 202 | 27.860 | 4.300 | 28.932 | 1.00 | 30.76 | O |
| ATOM | 3257 | C | SER | L | 202 | 27.903 | 1.506 | 31.442 | 1.00 | 29.24 | C |
| ATOM | 3258 | O | SER | L | 202 | 28.634 | 1.448 | 32.434 | 1.00 | 29.45 | O |
| ATOM | 3259 | N | SER | L | 203 | 26.688 | 0.970 | 31.390 | 1.00 | 29.26 | N |
| ATOM | 3260 | CA | SER | L | 203 | 26.140 | 0.146 | 32.456 | 1.00 | 29.44 | C |
| ATOM | 3261 | CB | SER | L | 203 | 25.253 | 0.991 | 33.377 | 1.00 | 29.36 | C |
| ATOM | 3262 | OG | SER | L | 203 | 24.085 | 1.427 | 32.702 | 1.00 | 30.26 | O |
| ATOM | 3263 | C | SER | L | 203 | 25.351 | −1.012 | 31.818 | 1.00 | 29.31 | C |
| ATOM | 3264 | O | SER | L | 203 | 25.080 | −0.974 | 30.614 | 1.00 | 28.93 | O |
| ATOM | 3265 | N | PRO | L | 204 | 24.982 | −2.039 | 32.619 | 1.00 | 29.20 | N |
| ATOM | 3266 | CA | PRO | L | 204 | 24.283 | −3.201 | 32.070 | 1.00 | 28.99 | C |
| ATOM | 3267 | CB | PRO | L | 204 | 23.932 | −4.009 | 33.323 | 1.00 | 29.01 | C |
| ATOM | 3268 | CG | PRO | L | 204 | 25.050 | −3.690 | 34.266 | 1.00 | 29.54 | C |
| ATOM | 3269 | CD | PRO | L | 204 | 25.248 | −2.212 | 34.061 | 1.00 | 29.27 | C |
| ATOM | 3270 | C | PRO | L | 204 | 23.021 | −2.839 | 31.299 | 1.00 | 28.74 | C |
| ATOM | 3271 | O | PRO | L | 204 | 22.255 | −1.989 | 31.741 | 1.00 | 28.33 | O |
| ATOM | 3272 | N | VAL | L | 205 | 22.826 | −3.479 | 30.149 | 1.00 | 28.45 | N |
| ATOM | 3273 | CA | VAL | L | 205 | 21.642 | −3.252 | 29.311 | 1.00 | 28.38 | C |
| ATOM | 3274 | CB | VAL | L | 205 | 22.033 | −3.040 | 27.828 | 1.00 | 28.72 | C |
| ATOM | 3275 | CG1 | VAL | L | 205 | 20.879 | −3.388 | 26.878 | 1.00 | 28.50 | C |
| ATOM | 3276 | CG2 | VAL | L | 205 | 22.486 | −1.607 | 27.619 | 1.00 | 29.40 | C |
| ATOM | 3277 | C | VAL | L | 205 | 20.654 | −4.407 | 29.461 | 1.00 | 27.86 | C |
| ATOM | 3278 | O | VAL | L | 205 | 21.056 | −5.571 | 29.481 | 1.00 | 27.67 | O |
| ATOM | 3279 | N | THR | L | 206 | 19.369 | −4.072 | 29.573 | 1.00 | 27.45 | N |
| ATOM | 3280 | CA | THR | L | 206 | 18.310 | −5.050 | 29.821 | 1.00 | 26.86 | C |
| ATOM | 3281 | CB | THR | L | 206 | 17.637 | −4.819 | 31.216 | 1.00 | 26.68 | C |
| ATOM | 3282 | OG1 | THR | L | 206 | 18.610 | −4.965 | 32.254 | 1.00 | 25.88 | O |
| ATOM | 3283 | CG2 | THR | L | 206 | 16.519 | −5.813 | 31.464 | 1.00 | 26.03 | C |
| ATOM | 3284 | C | THR | L | 206 | 17.241 | −4.974 | 28.736 | 1.00 | 26.94 | C |
| ATOM | 3285 | O | THR | L | 206 | 16.712 | −3.900 | 28.466 | 1.00 | 27.11 | O |
| ATOM | 3286 | N | LYS | L | 207 | 16.944 | −6.110 | 28.110 | 1.00 | 26.83 | N |
| ATOM | 3287 | CA | LYS | L | 207 | 15.754 | −6.239 | 27.273 | 1.00 | 26.53 | C |
| ATOM | 3288 | CB | LYS | L | 207 | 16.079 | −6.825 | 25.893 | 1.00 | 27.03 | C |
| ATOM | 3289 | CG | LYS | L | 207 | 17.113 | −6.071 | 25.083 | 1.00 | 26.35 | C |
| ATOM | 3290 | CD | LYS | L | 207 | 16.643 | −4.709 | 24.600 | 1.00 | 26.53 | C |
| ATOM | 3291 | CE | LYS | L | 207 | 17.841 | −3.942 | 24.055 | 1.00 | 27.09 | C |
| ATOM | 3292 | NZ | LYS | L | 207 | 17.644 | −2.476 | 24.033 | 1.00 | 27.83 | N |
| ATOM | 3293 | C | LYS | L | 207 | 14.804 | −7.167 | 27.997 | 1.00 | 26.37 | C |
| ATOM | 3294 | O | LYS | L | 207 | 15.231 | −8.136 | 28.625 | 1.00 | 26.51 | O |
| ATOM | 3295 | N | SER | L | 208 | 13.513 | −6.866 | 27.915 | 1.00 | 26.26 | N |
| ATOM | 3296 | CA | SER | L | 208 | 12.498 | −7.662 | 28.584 | 1.00 | 25.92 | C |
| ATOM | 3297 | CB | SER | L | 208 | 12.290 | −7.172 | 30.023 | 1.00 | 26.05 | C |
| ATOM | 3298 | OG | SER | L | 208 | 11.769 | −5.859 | 30.031 | 1.00 | 25.92 | O |
| ATOM | 3299 | C | SER | L | 208 | 11.182 | −7.613 | 27.831 | 1.00 | 25.91 | C |
| ATOM | 3300 | O | SER | L | 208 | 10.990 | −6.789 | 26.935 | 1.00 | 25.74 | O |
| ATOM | 3301 | N | PHE | L | 209 | 10.300 | −8.536 | 28.183 | 1.00 | 25.56 | N |
| ATOM | 3302 | CA | PHE | L | 209 | 8.927 | −8.514 | 27.734 | 1.00 | 25.74 | C |
| ATOM | 3303 | CB | PHE | L | 209 | 8.715 | −9.385 | 26.478 | 1.00 | 25.24 | C |
| ATOM | 3304 | CG | PHE | L | 209 | 9.005 | −10.852 | 26.684 | 1.00 | 23.35 | C |
| ATOM | 3305 | CD1 | PHE | L | 209 | 8.016 | −11.719 | 27.148 | 1.00 | 20.81 | C |
| ATOM | 3306 | CE1 | PHE | L | 209 | 8.280 | −13.073 | 27.334 | 1.00 | 19.70 | C |
| ATOM | 3307 | CZ | PHE | L | 209 | 9.561 | −13.577 | 27.051 | 1.00 | 20.80 | C |
| ATOM | 3308 | CE2 | PHE | L | 209 | 10.551 | −12.719 | 26.590 | 1.00 | 19.12 | C |
| ATOM | 3309 | CD2 | PHE | L | 209 | 10.272 | −11.369 | 26.405 | 1.00 | 20.83 | C |
| ATOM | 3310 | C | PHE | L | 209 | 8.083 | −9.004 | 28.886 | 1.00 | 26.35 | C |
| ATOM | 3311 | O | PHE | L | 209 | 8.592 | −9.651 | 29.813 | 1.00 | 26.07 | O |
| ATOM | 3312 | N | ASN | L | 210 | 6.802 | −8.657 | 28.840 | 1.00 | 27.18 | N |
| ATOM | 3313 | CA | ASN | L | 210 | 5.813 | −9.264 | 29.701 | 1.00 | 28.02 | C |
| ATOM | 3314 | CB | ASN | L | 210 | 4.806 | −8.217 | 30.199 | 1.00 | 28.29 | C |
| ATOM | 3315 | CG | ASN | L | 210 | 5.487 | −6.998 | 30.811 | 1.00 | 28.75 | C |
| ATOM | 3316 | OD1 | ASN | L | 210 | 6.335 | −7.116 | 31.701 | 1.00 | 27.41 | O |
| ATOM | 3317 | ND2 | ASN | L | 210 | 5.118 | −5.816 | 30.325 | 1.00 | 30.19 | N |
| ATOM | 3318 | C | ASN | L | 210 | 5.125 | −10.368 | 28.913 | 1.00 | 28.36 | C |
| ATOM | 3319 | O | ASN | L | 210 | 4.689 | −10.156 | 27.772 | 1.00 | 27.87 | O |
| ATOM | 3320 | N | ARG | L | 211 | 5.067 | −11.551 | 29.515 | 1.00 | 29.09 | N |
| ATOM | 3321 | CA | ARG | L | 211 | 4.413 | −12.700 | 28.917 | 1.00 | 30.36 | C |
| ATOM | 3322 | CB | ARG | L | 211 | 4.417 | −13.853 | 29.917 | 1.00 | 30.10 | C |
| ATOM | 3323 | CG | ARG | L | 211 | 3.837 | −15.160 | 29.399 | 1.00 | 30.06 | C |
| ATOM | 3324 | CD | ARG | L | 211 | 3.607 | −16.146 | 30.536 | 1.00 | 29.84 | C |
| ATOM | 3325 | NE | ARG | L | 211 | 3.100 | −15.478 | 31.737 | 1.00 | 30.24 | N |
| ATOM | 3326 | CZ | ARG | L | 211 | 1.876 | −14.963 | 31.871 | 1.00 | 30.58 | C |
| ATOM | 3327 | NH1 | ARG | L | 211 | 0.993 | −15.033 | 30.881 | 1.00 | 31.26 | N |
| ATOM | 3328 | NH2 | ARG | L | 211 | 1.537 | −14.367 | 33.002 | 1.00 | 29.76 | N |
| ATOM | 3329 | C | ARG | L | 211 | 2.988 | −12.301 | 28.517 | 1.00 | 31.46 | C |
| ATOM | 3330 | O | ARG | L | 211 | 2.174 | −11.955 | 29.372 | 1.00 | 31.38 | O |
| ATOM | 3331 | N | GLY | L | 212 | 2.712 | −12.329 | 27.212 | 1.00 | 32.71 | N |
| ATOM | 3332 | CA | GLY | L | 212 | 1.484 | −11.760 | 26.638 | 1.00 | 33.90 | C |
| ATOM | 3333 | C | GLY | L | 212 | 1.844 | −10.689 | 25.616 | 1.00 | 34.86 | C |
| ATOM | 3334 | O | GLY | L | 212 | 2.675 | −10.921 | 24.738 | 1.00 | 35.20 | O |
| ATOM | 3335 | N | GLU | L | 213 | 1.225 | −9.515 | 25.737 | 1.00 | 35.96 | N |
| ATOM | 3336 | CA | GLU | L | 213 | 1.545 | −8.334 | 24.907 | 1.00 | 36.61 | C |
| ATOM | 3337 | CB | GLU | L | 213 | 2.520 | −7.414 | 25.652 | 1.00 | 36.79 | C |
| ATOM | 3338 | CG | GLU | L | 213 | 1.919 | −6.694 | 26.867 | 1.00 | 37.25 | C |
| ATOM | 3339 | CD | GLU | L | 213 | 2.899 | −5.735 | 27.543 | 1.00 | 38.16 | C |
| ATOM | 3340 | OE1 | GLU | L | 213 | 4.115 | −5.800 | 27.256 | 1.00 | 38.23 | O |
| ATOM | 3341 | OE2 | GLU | L | 213 | 2.453 | −4.914 | 28.372 | 1.00 | 38.66 | O |
| ATOM | 3342 | C | GLU | L | 213 | 2.089 | −8.641 | 23.503 | 1.00 | 36.92 | C |
| ATOM | 3343 | O | GLU | L | 213 | 1.567 | −8.156 | 22.493 | 1.00 | 37.19 | O |
| ATOM | 3344 | N | GLU | I | 1 | 31.609 | −36.487 | 3.964 | 1.00 | 36.74 | N |
| ATOM | 3345 | CA | GLU | I | 1 | 31.305 | −36.150 | 2.547 | 1.00 | 36.65 | C |
| ATOM | 3346 | CB | GLU | I | 1 | 32.007 | −34.843 | 2.133 | 1.00 | 37.37 | C |
| ATOM | 3347 | CG | GLU | I | 1 | 31.570 | −33.596 | 2.910 | 1.00 | 40.67 | C |
| ATOM | 3348 | CD | GLU | I | 1 | 30.061 | −33.337 | 2.838 | 1.00 | 44.70 | C |
| ATOM | 3349 | OE1 | GLU | I | 1 | 29.463 | −33.499 | 1.741 | 1.00 | 46.78 | O |
| ATOM | 3350 | OE2 | GLU | I | 1 | 29.479 | −32.967 | 3.881 | 1.00 | 45.29 | O |
| ATOM | 3351 | C | GLU | I | 1 | 31.708 | −37.272 | 1.584 | 1.00 | 35.51 | C |
| ATOM | 3352 | O | GLU | I | 1 | 32.266 | −38.303 | 1.995 | 1.00 | 35.83 | O |
| ATOM | 3353 | N | VAL | I | 2 | 31.401 | −37.062 | 0.306 | 1.00 | 33.29 | N |
| ATOM | 3354 | CA | VAL | I | 2 | 31.941 | −37.876 | −0.763 | 1.00 | 31.35 | C |
| ATOM | 3355 | CB | VAL | I | 2 | 31.009 | −37.896 | −2.005 | 1.00 | 31.50 | C |
| ATOM | 3356 | CG1 | VAL | I | 2 | 31.687 | −38.551 | −3.204 | 1.00 | 31.16 | C |
| ATOM | 3357 | CG2 | VAL | I | 2 | 29.714 | −38.613 | −1.670 | 1.00 | 32.50 | C |
| ATOM | 3358 | C | VAL | I | 2 | 33.317 | −37.309 | −1.107 | 1.00 | 29.52 | C |
| ATOM | 3359 | O | VAL | I | 2 | 33.511 | −36.092 | −1.086 | 1.00 | 29.03 | O |
| ATOM | 3360 | N | GLN | I | 3 | 34.272 | −38.196 | −1.365 | 1.00 | 27.10 | N |
| ATOM | 3361 | CA | GLN | I | 3 | 35.594 | −37.792 | −1.840 | 1.00 | 25.89 | C |
| ATOM | 3362 | CB | GLN | I | 3 | 36.663 | −38.086 | −0.782 | 1.00 | 25.65 | C |
| ATOM | 3363 | CG | GLN | I | 3 | 36.438 | −37.351 | 0.542 | 1.00 | 28.88 | C |
| ATOM | 3364 | CD | GLN | I | 3 | 37.679 | −37.307 | 1.406 | 1.00 | 31.16 | C |
| ATOM | 3365 | OE1 | GLN | I | 3 | 38.658 | −36.634 | 1.085 | 1.00 | 32.79 | O |
| ATOM | 3366 | NE2 | GLN | I | 3 | 37.643 | −38.024 | 2.509 | 1.00 | 31.27 | N |
| ATOM | 3367 | C | GLN | I | 3 | 35.911 | −38.561 | −3.112 | 1.00 | 24.02 | C |
| ATOM | 3368 | O | GLN | I | 3 | 35.771 | −39.792 | −3.150 | 1.00 | 24.22 | O |
| ATOM | 3369 | N | LEU | I | 4 | 36.320 | −37.847 | −4.146 | 1.00 | 21.56 | N |
| ATOM | 3370 | CA | LEU | I | 4 | 36.869 | −38.498 | −5.328 | 1.00 | 20.55 | C |
| ATOM | 3371 | CB | LEU | I | 4 | 36.367 | −37.819 | −6.600 | 1.00 | 19.93 | C |
| ATOM | 3372 | CG | LEU | I | 4 | 34.855 | −37.667 | −6.767 | 1.00 | 19.08 | C |
| ATOM | 3373 | CD1 | LEU | I | 4 | 34.557 | −37.016 | −8.092 | 1.00 | 16.81 | C |
| ATOM | 3374 | CD2 | LEU | I | 4 | 34.134 | −39.006 | −6.641 | 1.00 | 17.29 | C |
| ATOM | 3375 | C | LEU | I | 4 | 38.395 | −38.481 | −5.267 | 1.00 | 20.09 | C |
| ATOM | 3376 | O | LEU | I | 4 | 39.001 | −37.409 | −5.145 | 1.00 | 19.01 | O |
| ATOM | 3377 | N | VAL | I | 5 | 39.017 | −39.658 | −5.325 | 1.00 | 19.86 | N |
| ATOM | 3378 | CA | VAL | I | 5 | 40.481 | −39.731 | −5.234 | 1.00 | 20.35 | C |
| ATOM | 3379 | CB | VAL | I | 5 | 40.961 | −40.595 | −4.021 | 1.00 | 20.68 | C |
| ATOM | 3380 | CG1 | VAL | I | 5 | 42.507 | −40.585 | −3.928 | 1.00 | 20.48 | C |
| ATOM | 3381 | CG2 | VAL | I | 5 | 40.350 | −40.078 | −2.719 | 1.00 | 20.80 | C |
| ATOM | 3382 | C | VAL | I | 5 | 41.096 | −40.283 | −6.515 | 1.00 | 20.10 | C |
| ATOM | 3383 | O | VAL | I | 5 | 40.849 | −41.422 | −6.874 | 1.00 | 19.83 | O |
| ATOM | 3384 | N | GLN | I | 6 | 41.922 | −39.483 | −7.179 | 1.00 | 20.45 | N |
| ATOM | 3385 | CA | GLN | I | 6 | 42.507 | −39.891 | −8.464 | 1.00 | 20.68 | C |
| ATOM | 3386 | CB | GLN | I | 6 | 42.487 | −38.728 | −9.442 | 1.00 | 20.76 | C |
| ATOM | 3387 | CG | GLN | I | 6 | 41.091 | −38.246 | −9.742 | 1.00 | 20.60 | C |
| ATOM | 3388 | CD | GLN | I | 6 | 41.062 | −37.101 | −10.711 | 1.00 | 20.33 | C |
| ATOM | 3389 | OE1 | GLN | I | 6 | 40.493 | −36.050 | −10.428 | 1.00 | 20.16 | O |
| ATOM | 3390 | NE2 | GLN | I | 6 | 41.679 | −37.287 | −11.871 | 1.00 | 21.53 | N |
| ATOM | 3391 | C | GLN | I | 6 | 43.918 | −40.472 | −8.338 | 1.00 | 21.47 | C |
| ATOM | 3392 | O | GLN | I | 6 | 44.663 | −40.127 | −7.410 | 1.00 | 21.87 | O |
| ATOM | 3393 | N | SER | I | 7 | 44.262 | −41.373 | −9.261 | 1.00 | 21.59 | N |
| ATOM | 3394 | CA | SER | I | 7 | 45.610 | −41.966 | −9.360 | 1.00 | 21.85 | C |
| ATOM | 3395 | CB | SER | I | 7 | 45.647 | −43.044 | −10.469 | 1.00 | 21.43 | C |
| ATOM | 3396 | OG | SER | I | 7 | 44.998 | −42.585 | −11.650 | 1.00 | 19.91 | O |
| ATOM | 3397 | C | SER | I | 7 | 46.687 | −40.902 | −9.606 | 1.00 | 22.13 | C |
| ATOM | 3398 | O | SER | I | 7 | 46.375 | −39.761 | −9.963 | 1.00 | 22.54 | O |
| ATOM | 3399 | N | GLY | I | 8 | 47.954 | −41.279 | −9.416 | 1.00 | 22.53 | N |
| ATOM | 3400 | CA | GLY | I | 8 | 49.066 | −40.312 | −9.481 | 1.00 | 22.84 | C |
| ATOM | 3401 | C | GLY | I | 8 | 49.442 | −39.835 | −10.878 | 1.00 | 22.59 | C |
| ATOM | 3402 | O | GLY | I | 8 | 48.863 | −40.274 | −11.876 | 1.00 | 22.61 | O |
| ATOM | 3403 | N | ALA | I | 9 | 50.421 | −38.939 | −10.956 | 1.00 | 22.03 | N |
| ATOM | 3404 | CA | ALA | I | 9 | 50.804 | −38.352 | −12.257 | 1.00 | 21.64 | C |
| ATOM | 3405 | CB | ALA | I | 9 | 51.819 | −37.212 | −12.088 | 1.00 | 21.88 | C |
| ATOM | 3406 | C | ALA | I | 9 | 51.334 | −39.410 | −13.223 | 1.00 | 20.98 | C |
| ATOM | 3407 | O | ALA | I | 9 | 51.930 | −40.421 | −12.800 | 1.00 | 20.53 | O |
| ATOM | 3408 | N | GLU | I | 10 | 51.071 | −39.180 | −14.509 | 1.00 | 20.55 | N |
| ATOM | 3409 | CA | GLU | I | 10 | 51.489 | −40.067 | −15.605 | 1.00 | 20.81 | C |
| ATOM | 3410 | CB | GLU | I | 10 | 50.289 | −40.496 | −16.455 | 1.00 | 21.31 | C |
| ATOM | 3411 | CG | GLU | I | 10 | 49.172 | −41.218 | −15.697 | 1.00 | 23.85 | C |
| ATOM | 3412 | CD | GLU | I | 10 | 49.466 | −42.680 | −15.419 | 1.00 | 25.99 | C |
| ATOM | 3413 | OE1 | GLU | I | 10 | 50.478 | −43.206 | −15.934 | 1.00 | 28.21 | O |
| ATOM | 3414 | OE2 | GLU | I | 10 | 48.668 | −43.314 | −14.691 | 1.00 | 28.67 | O |
| ATOM | 3415 | C | GLU | I | 10 | 52.453 | −39.342 | −16.528 | 1.00 | 20.75 | C |
| ATOM | 3416 | O | GLU | I | 10 | 52.274 | −38.152 | −16.832 | 1.00 | 20.38 | O |
| ATOM | 3417 | N | VAL | I | 11 | 53.463 | −40.072 | −16.993 | 1.00 | 20.35 | N |
| ATOM | 3418 | CA | VAL | I | 11 | 54.384 | −39.549 | −17.991 | 1.00 | 20.42 | C |
| ATOM | 3419 | CB | VAL | I | 11 | 55.775 | −39.219 | −17.389 | 1.00 | 20.49 | C |
| ATOM | 3420 | CG1 | VAL | I | 11 | 56.656 | −38.500 | −18.428 | 1.00 | 20.81 | C |
| ATOM | 3421 | CG2 | VAL | I | 11 | 55.629 | −38.390 | −16.105 | 1.00 | 21.38 | C |
| ATOM | 3422 | C | VAL | I | 11 | 54.491 | −40.632 | −19.046 | 1.00 | 20.30 | C |
| ATOM | 3423 | O | VAL | I | 11 | 54.937 | −41.754 | −18.759 | 1.00 | 20.73 | O |
| ATOM | 3424 | N | LYS | I | 12 | 54.057 | −40.292 | −20.255 | 1.00 | 19.62 | N |
| ATOM | 3425 | CA | LYS | I | 12 | 53.935 | −41.251 | −21.345 | 1.00 | 20.03 | C |
| ATOM | 3426 | CB | LYS | I | 12 | 52.452 | −41.479 | −21.668 | 1.00 | 19.60 | C |
| ATOM | 3427 | CG | LYS | I | 12 | 51.674 | −42.114 | −20.525 | 1.00 | 19.87 | C |
| ATOM | 3428 | CD | LYS | I | 12 | 51.932 | −43.616 | −20.515 | 1.00 | 24.31 | C |
| ATOM | 3429 | CE | LYS | I | 12 | 51.785 | −44.182 | −19.131 | 1.00 | 24.04 | C |
| ATOM | 3430 | NZ | LYS | I | 12 | 51.894 | −45.659 | −19.182 | 1.00 | 25.73 | N |
| ATOM | 3431 | C | LYS | I | 12 | 54.639 | −40.777 | −22.600 | 1.00 | 20.31 | C |
| ATOM | 3432 | O | LYS | I | 12 | 54.812 | −39.576 | −22.795 | 1.00 | 20.35 | O |
| ATOM | 3433 | N | LYS | I | 13 | 55.040 | −41.732 | −23.442 | 1.00 | 20.28 | N |
| ATOM | 3434 | CA | LYS | I | 13 | 55.544 | −41.435 | −24.779 | 1.00 | 21.02 | C |
| ATOM | 3435 | CB | LYS | I | 13 | 56.569 | −42.496 | −25.214 | 1.00 | 21.13 | C |
| ATOM | 3436 | CG | LYS | I | 13 | 57.814 | −42.587 | −24.346 | 1.00 | 22.01 | C |
| ATOM | 3437 | CD | LYS | I | 13 | 58.894 | −41.664 | −24.864 | 1.00 | 25.43 | C |
| ATOM | 3438 | CE | LYS | I | 13 | 59.915 | −41.372 | −23.805 | 1.00 | 26.50 | C |
| ATOM | 3439 | NZ | LYS | I | 13 | 60.910 | −42.451 | −23.660 | 1.00 | 28.78 | N |
| ATOM | 3440 | C | LYS | I | 13 | 54.360 | −41.437 | −25.750 | 1.00 | 21.22 | C |
| ATOM | 3441 | O | LYS | I | 13 | 53.355 | −42.107 | −25.495 | 1.00 | 21.02 | O |
| ATOM | 3442 | N | PRO | I | 14 | 54.471 | −40.696 | −26.874 | 1.00 | 21.41 | N |
| ATOM | 3443 | CA | PRO | I | 14 | 53.434 | −40.736 | −27.914 | 1.00 | 20.85 | C |
| ATOM | 3444 | CB | PRO | I | 14 | 54.044 | −39.915 | −29.050 | 1.00 | 20.83 | C |
| ATOM | 3445 | CG | PRO | I | 14 | 55.016 | −39.010 | −28.386 | 1.00 | 21.72 | C |
| ATOM | 3446 | CD | PRO | I | 14 | 55.601 | −39.835 | −27.272 | 1.00 | 21.80 | C |
| ATOM | 3447 | C | PRO | I | 14 | 53.169 | −42.159 | −28.405 | 1.00 | 20.57 | C |
| ATOM | 3448 | O | PRO | I | 14 | 54.101 | −42.956 | −28.507 | 1.00 | 19.92 | O |
| ATOM | 3449 | N | GLY | I | 15 | 51.900 | −42.465 | −28.684 | 1.00 | 20.34 | N |
| ATOM | 3450 | CA | GLY | I | 15 | 51.474 | −43.801 | −29.112 | 1.00 | 20.51 | C |
| ATOM | 3451 | C | GLY | I | 15 | 51.108 | −44.768 | −27.983 | 1.00 | 20.12 | C |
| ATOM | 3452 | O | GLY | I | 15 | 50.439 | −45.770 | −28.215 | 1.00 | 19.62 | O |
| ATOM | 3453 | N | GLU | I | 16 | 51.561 | −44.478 | −26.764 | 1.00 | 20.04 | N |
| ATOM | 3454 | CA | GLU | I | 16 | 51.276 | −45.333 | −25.626 | 1.00 | 20.07 | C |
| ATOM | 3455 | CB | GLU | I | 16 | 52.260 | −45.049 | −24.477 | 1.00 | 20.59 | C |
| ATOM | 3456 | CG | GLU | I | 16 | 53.699 | −45.449 | −24.830 | 1.00 | 21.41 | C |
| ATOM | 3457 | CD | GLU | I | 16 | 54.674 | −45.332 | −23.675 | 1.00 | 23.04 | C |
| ATOM | 3458 | OE1 | GLU | I | 16 | 55.626 | −46.140 | −23.650 | 1.00 | 26.63 | O |
| ATOM | 3459 | OE2 | GLU | I | 16 | 54.521 | −44.450 | −22.807 | 1.00 | 19.77 | O |
| ATOM | 3460 | C | GLU | I | 16 | 49.831 | −45.144 | −25.176 | 1.00 | 20.19 | C |
| ATOM | 3461 | O | GLU | I | 16 | 49.108 | −44.276 | −25.692 | 1.00 | 19.10 | O |
| ATOM | 3462 | N | SER | I | 17 | 49.414 | −45.965 | −24.220 | 1.00 | 20.38 | N |
| ATOM | 3463 | CA | SER | I | 17 | 48.121 | −45.799 | −23.570 | 1.00 | 20.74 | C |
| ATOM | 3464 | CB | SER | I | 17 | 47.266 | −47.048 | −23.755 | 1.00 | 21.06 | C |
| ATOM | 3465 | OG | SER | I | 17 | 46.947 | −47.204 | −25.115 | 1.00 | 22.50 | O |
| ATOM | 3466 | C | SER | I | 17 | 48.279 | −45.529 | −22.093 | 1.00 | 20.87 | C |
| ATOM | 3467 | O | SER | I | 17 | 49.314 | −45.859 | −21.503 | 1.00 | 21.11 | O |
| ATOM | 3468 | N | LEU | I | 18 | 47.239 | −44.936 | −21.510 | 1.00 | 20.35 | N |
| ATOM | 3469 | CA | LEU | I | 18 | 47.161 | −44.736 | −20.072 | 1.00 | 20.43 | C |
| ATOM | 3470 | CB | LEU | I | 18 | 47.489 | −43.278 | −19.683 | 1.00 | 19.60 | C |
| ATOM | 3471 | CG | LEU | I | 18 | 46.658 | −42.103 | −20.205 | 1.00 | 19.44 | C |
| ATOM | 3472 | CD1 | LEU | I | 18 | 45.299 | −42.029 | −19.547 | 1.00 | 20.98 | C |
| ATOM | 3473 | CD2 | LEU | I | 18 | 47.377 | −40.820 | −19.938 | 1.00 | 20.74 | C |
| ATOM | 3474 | C | LEU | I | 18 | 45.781 | −45.157 | −19.570 | 1.00 | 20.26 | C |
| ATOM | 3475 | O | LEU | I | 18 | 44.823 | −45.297 | −20.348 | 1.00 | 20.27 | O |
| ATOM | 3476 | N | LYS | I | 19 | 45.696 | −45.376 | −18.269 | 1.00 | 20.36 | N |
| ATOM | 3477 | CA | LYS | I | 19 | 44.436 | −45.657 | −17.630 | 1.00 | 20.02 | C |
| ATOM | 3478 | CB | LYS | I | 19 | 44.193 | −47.165 | −17.519 | 1.00 | 20.22 | C |
| ATOM | 3479 | CG | LYS | I | 19 | 42.828 | −47.523 | −16.969 | 1.00 | 20.90 | C |
| ATOM | 3480 | CD | LYS | I | 19 | 42.557 | −49.031 | −17.063 | 1.00 | 22.99 | C |
| ATOM | 3481 | CE | LYS | I | 19 | 41.073 | −49.329 | −16.828 | 1.00 | 23.40 | C |
| ATOM | 3482 | NZ | LYS | I | 19 | 40.781 | −50.801 | −16.972 | 1.00 | 24.19 | N |
| ATOM | 3483 | C | LYS | I | 19 | 44.527 | −45.033 | −16.275 | 1.00 | 20.11 | C |
| ATOM | 3484 | O | LYS | I | 19 | 45.362 | −45.420 | −15.457 | 1.00 | 20.41 | O |
| ATOM | 3485 | N | ILE | I | 20 | 43.670 | −44.052 | −16.040 | 1.00 | 19.80 | N |
| ATOM | 3486 | CA | ILE | I | 20 | 43.678 | −43.333 | −14.778 | 1.00 | 20.12 | C |
| ATOM | 3487 | CB | ILE | I | 20 | 44.011 | −41.832 | −14.978 | 1.00 | 20.27 | C |
| ATOM | 3488 | CG1 | ILE | I | 20 | 43.022 | −41.147 | −15.917 | 1.00 | 20.71 | C |
| ATOM | 3489 | CD1 | ILE | I | 20 | 43.340 | −39.638 | −16.128 | 1.00 | 19.78 | C |
| ATOM | 3490 | CG2 | ILE | I | 20 | 45.417 | −41.694 | −15.601 | 1.00 | 21.35 | C |
| ATOM | 3491 | C | ILE | I | 20 | 42.377 | −43.597 | −14.008 | 1.00 | 19.98 | C |
| ATOM | 3492 | O | ILE | I | 20 | 41.332 | −43.888 | −14.608 | 1.00 | 19.37 | O |
| ATOM | 3493 | N | SER | I | 21 | 42.465 | −43.563 | −12.682 | 1.00 | 19.68 | N |
| ATOM | 3494 | CA | SER | I | 21 | 41.342 | −43.976 | −11.843 | 1.00 | 19.02 | C |
| ATOM | 3495 | CB | SER | I | 21 | 41.748 | −45.103 | −10.893 | 1.00 | 19.36 | C |
| ATOM | 3496 | OG | SER | I | 21 | 42.822 | −44.712 | −10.041 | 1.00 | 19.16 | O |
| ATOM | 3497 | C | SER | I | 21 | 40.786 | −42.803 | −11.062 | 1.00 | 19.79 | C |
| ATOM | 3498 | O | SER | I | 21 | 41.467 | −41.782 | −10.839 | 1.00 | 18.81 | O |
| ATOM | 3499 | N | CYS | I | 22 | 39.530 | −42.955 | −10.669 | 1.00 | 19.29 | N |
| ATOM | 3500 | CA | CYS | I | 22 | 38.844 | −41.979 | −9.859 | 1.00 | 20.06 | C |
| ATOM | 3501 | CB | CYS | I | 22 | 37.969 | −41.106 | −10.757 | 1.00 | 20.13 | C |
| ATOM | 3502 | SG | CYS | I | 22 | 36.839 | −40.019 | −9.877 | 1.00 | 21.64 | S |
| ATOM | 3503 | C | CYS | I | 22 | 38.000 | −42.796 | −8.887 | 1.00 | 19.64 | C |
| ATOM | 3504 | O | CYS | I | 22 | 36.945 | −43.307 | −9.252 | 1.00 | 19.74 | O |
| ATOM | 3505 | N | GLN | I | 23 | 38.484 | −42.963 | −7.669 | 1.00 | 20.06 | N |
| ATOM | 3506 | CA | GLN | I | 23 | 37.781 | −43.802 | −6.709 | 1.00 | 20.82 | C |
| ATOM | 3507 | CB | GLN | I | 23 | 38.760 | −44.604 | −5.855 | 1.00 | 20.84 | C |
| ATOM | 3508 | CG | GLN | I | 23 | 38.069 | −45.622 | −4.945 | 1.00 | 21.88 | C |
| ATOM | 3509 | CD | GLN | I | 23 | 39.036 | −46.583 | −4.274 | 1.00 | 22.70 | C |
| ATOM | 3510 | OE1 | GLN | I | 23 | 40.188 | −46.251 | −4.011 | 1.00 | 21.40 | O |
| ATOM | 3511 | NE2 | GLN | I | 23 | 38.555 | −47.780 | −3.977 | 1.00 | 23.48 | N |
| ATOM | 3512 | C | GLN | I | 23 | 36.879 | −42.932 | −5.848 | 1.00 | 21.48 | C |
| ATOM | 3513 | O | GLN | I | 23 | 37.318 | −41.936 | −5.272 | 1.00 | 21.77 | O |
| ATOM | 3514 | N | ALA | I | 24 | 35.614 | −43.314 | −5.770 | 1.00 | 21.86 | N |
| ATOM | 3515 | CA | ALA | I | 24 | 34.621 | −42.531 | −5.067 | 1.00 | 22.23 | C |
| ATOM | 3516 | CB | ALA | I | 24 | 33.355 | −42.455 | −5.881 | 1.00 | 22.82 | C |
| ATOM | 3517 | C | ALA | I | 24 | 34.363 | −43.148 | −3.710 | 1.00 | 22.90 | C |
| ATOM | 3518 | O | ALA | I | 24 | 34.110 | −44.345 | −3.597 | 1.00 | 21.73 | O |
| ATOM | 3519 | N | PHE | I | 25 | 34.502 | −42.324 | −2.677 | 1.00 | 23.62 | N |
| ATOM | 3520 | CA | PHE | I | 25 | 34.254 | −42.737 | −1.303 | 1.00 | 24.81 | C |
| ATOM | 3521 | CB | PHE | I | 25 | 35.418 | −42.325 | −0.404 | 1.00 | 24.25 | C |
| ATOM | 3522 | CG | PHE | I | 25 | 36.662 | −43.092 | −0.675 | 1.00 | 24.45 | C |
| ATOM | 3523 | CD1 | PHE | I | 25 | 36.944 | −44.242 | 0.050 | 1.00 | 24.01 | C |
| ATOM | 3524 | CE1 | PHE | I | 25 | 38.091 | −44.972 | −0.211 | 1.00 | 25.54 | C |
| ATOM | 3525 | CZ | PHE | I | 25 | 38.963 | −44.555 | −1.206 | 1.00 | 23.43 | C |
| ATOM | 3526 | CE2 | PHE | I | 25 | 38.690 | −43.407 | −1.931 | 1.00 | 24.69 | C |
| ATOM | 3527 | CD2 | PHE | I | 25 | 37.541 | −42.688 | −1.676 | 1.00 | 24.20 | C |
| ATOM | 3528 | C | PHE | I | 25 | 32.980 | −42.070 | −0.856 | 1.00 | 26.16 | C |
| ATOM | 3529 | O | PHE | I | 25 | 32.909 | −40.836 | −0.786 | 1.00 | 26.38 | O |
| ATOM | 3530 | N | GLY | I | 26 | 31.965 | −42.883 | −0.591 | 1.00 | 27.18 | N |
| ATOM | 3531 | CA | GLY | I | 26 | 30.670 | −42.363 | −0.190 | 1.00 | 29.22 | C |
| ATOM | 3532 | C | GLY | I | 26 | 29.556 | −43.153 | −0.825 | 1.00 | 30.51 | C |
| ATOM | 3533 | O | GLY | I | 26 | 29.793 | −44.074 | −1.605 | 1.00 | 30.46 | O |
| ATOM | 3534 | N | TYR | I | 27 | 28.330 | −42.779 | −0.492 | 1.00 | 31.48 | N |
| ATOM | 3535 | CA | TYR | I | 27 | 27.162 | −43.537 | −0.912 | 1.00 | 32.48 | C |
| ATOM | 3536 | CB | TYR | I | 27 | 25.974 | −43.173 | −0.022 | 1.00 | 33.37 | C |
| ATOM | 3537 | CG | TYR | I | 27 | 26.145 | −43.568 | 1.435 | 1.00 | 37.21 | C |
| ATOM | 3538 | CD1 | TYR | I | 27 | 25.528 | −44.717 | 1.940 | 1.00 | 39.62 | C |
| ATOM | 3539 | CE1 | TYR | I | 27 | 25.666 | −45.085 | 3.285 | 1.00 | 41.60 | C |
| ATOM | 3540 | CZ | TYR | I | 27 | 26.438 | −44.297 | 4.133 | 1.00 | 41.88 | C |
| ATOM | 3541 | OH | TYR | I | 27 | 26.573 | −44.664 | 5.456 | 1.00 | 43.54 | O |
| ATOM | 3542 | CE2 | TYR | I | 27 | 27.064 | −43.144 | 3.658 | 1.00 | 40.97 | C |
| ATOM | 3543 | CD2 | TYR | I | 27 | 26.915 | −42.784 | 2.316 | 1.00 | 39.77 | C |
| ATOM | 3544 | C | TYR | I | 27 | 26.787 | −43.333 | −2.378 | 1.00 | 31.48 | C |
| ATOM | 3545 | O | TYR | I | 27 | 26.934 | −42.235 | −2.926 | 1.00 | 32.53 | O |
| ATOM | 3546 | N | GLY | I | 28 | 26.322 | −44.415 | −2.995 | 1.00 | 30.74 | N |
| ATOM | 3547 | CA | GLY | I | 28 | 25.564 | −44.369 | −4.245 | 1.00 | 29.07 | C |
| ATOM | 3548 | C | GLY | I | 28 | 26.374 | −44.233 | −5.514 | 1.00 | 27.66 | C |
| ATOM | 3549 | O | GLY | I | 28 | 25.995 | −43.484 | −6.416 | 1.00 | 28.17 | O |
| ATOM | 3550 | N | PHE | I | 29 | 27.491 | −44.942 | −5.595 | 1.00 | 25.64 | N |
| ATOM | 3551 | CA | PHE | I | 29 | 28.334 | −44.897 | −6.795 | 1.00 | 24.42 | C |
| ATOM | 3552 | CB | PHE | I | 29 | 29.486 | −45.887 | −6.624 | 1.00 | 24.15 | C |
| ATOM | 3553 | CG | PHE | I | 29 | 30.440 | −45.940 | −7.792 | 1.00 | 22.51 | C |
| ATOM | 3554 | CD1 | PHE | I | 29 | 31.290 | −44.868 | −8.073 | 1.00 | 21.96 | C |
| ATOM | 3555 | CE1 | PHE | I | 29 | 32.184 | −44.923 | −9.144 | 1.00 | 21.15 | C |
| ATOM | 3556 | CZ | PHE | I | 29 | 32.250 | −46.072 | −9.917 | 1.00 | 20.36 | C |
| ATOM | 3557 | CE2 | PHE | I | 29 | 31.430 | −47.148 | −9.640 | 1.00 | 19.03 | C |
| ATOM | 3558 | CD2 | PHE | I | 29 | 30.528 | −47.092 | −8.577 | 1.00 | 20.41 | C |
| ATOM | 3559 | C | PHE | I | 29 | 27.538 | −45.136 | −8.109 | 1.00 | 23.50 | C |
| ATOM | 3560 | O | PHE | I | 29 | 27.791 | −44.487 | −9.116 | 1.00 | 22.52 | O |
| ATOM | 3561 | N | ILE | I | 30 | 26.552 | −46.037 | −8.082 | 1.00 | 23.17 | N |
| ATOM | 3562 | CA | ILE | I | 30 | 25.747 | −46.363 | −9.287 | 1.00 | 23.17 | C |
| ATOM | 3563 | CB | ILE | I | 30 | 25.101 | −47.769 | −9.174 | 1.00 | 23.04 | C |
| ATOM | 3564 | CG1 | ILE | I | 30 | 24.124 | −47.821 | −7.989 | 1.00 | 22.38 | C |
| ATOM | 3565 | CD1 | ILE | I | 30 | 23.060 | −48.902 | −8.102 | 1.00 | 22.00 | C |
| ATOM | 3566 | CG2 | ILE | I | 30 | 26.177 | −48.853 | −9.051 | 1.00 | 22.56 | C |
| ATOM | 3567 | C | ILE | I | 30 | 24.630 | −45.353 | −9.591 | 1.00 | 23.93 | C |
| ATOM | 3568 | O | ILE | I | 30 | 23.992 | −45.411 | −10.635 | 1.00 | 24.00 | O |
| ATOM | 3569 | N | ASN | I | 31 | 24.364 | −44.455 | −8.657 | 1.00 | 24.88 | N |
| ATOM | 3570 | CA | ASN | I | 31 | 23.355 | −43.440 | −8.865 | 1.00 | 26.66 | C |
| ATOM | 3571 | CB | ASN | I | 31 | 22.671 | −43.057 | −7.544 | 1.00 | 27.13 | C |
| ATOM | 3572 | CG | ASN | I | 31 | 22.122 | −44.258 | −6.792 | 1.00 | 29.79 | C |
| ATOM | 3573 | OD1 | ASN | I | 31 | 22.670 | −44.665 | −5.774 | 1.00 | 33.70 | O |
| ATOM | 3574 | ND2 | ASN | I | 31 | 21.029 | −44.822 | −7.282 | 1.00 | 32.70 | N |
| ATOM | 3575 | C | ASN | I | 31 | 23.997 | −42.216 | −9.488 | 1.00 | 26.82 | C |
| ATOM | 3576 | O | ASN | I | 31 | 23.287 | −41.322 | −9.921 | 1.00 | 27.78 | O |
| ATOM | 3577 | N | TYR | I | 32 | 25.338 | −42.231 | −9.529 | 1.00 | 26.67 | N |
| ATOM | 3578 | CA | TYR | I | 32 | 26.231 | −41.114 | −9.889 | 1.00 | 26.49 | C |
| ATOM | 3579 | CB | TYR | I | 32 | 27.696 | −41.470 | −9.501 | 1.00 | 26.50 | C |
| ATOM | 3580 | CG | TYR | I | 32 | 28.103 | −41.411 | −8.035 | 1.00 | 30.47 | C |
| ATOM | 3581 | CD1 | TYR | I | 32 | 27.181 | −41.598 | −6.997 | 1.00 | 33.77 | C |
| ATOM | 3582 | CE1 | TYR | I | 32 | 27.592 | −41.553 | −5.651 | 1.00 | 35.10 | C |
| ATOM | 3583 | CZ | TYR | I | 32 | 28.935 | −41.349 | −5.346 | 1.00 | 35.49 | C |
| ATOM | 3584 | OH | TYR | I | 32 | 29.359 | −41.318 | −4.033 | 1.00 | 36.15 | O |
| ATOM | 3585 | CE2 | TYR | I | 32 | 29.854 | −41.155 | −6.363 | 1.00 | 33.75 | C |
| ATOM | 3586 | CD2 | TYR | I | 32 | 29.443 | −41.192 | −7.684 | 1.00 | 32.73 | C |
| ATOM | 3587 | C | TYR | I | 32 | 26.288 | −40.869 | −11.398 | 1.00 | 24.06 | C |
| ATOM | 3588 | O | TYR | I | 32 | 26.284 | −41.817 | −12.136 | 1.00 | 22.65 | O |
| ATOM | 3589 | N | LEU | I | 33 | 26.395 | −39.612 | −11.842 | 1.00 | 22.79 | N |
| ATOM | 3590 | CA | LEU | I | 33 | 27.011 | −39.352 | −13.158 | 1.00 | 22.55 | C |
| ATOM | 3591 | CB | LEU | I | 33 | 26.214 | −38.363 | −14.020 | 1.00 | 22.59 | C |
| ATOM | 3592 | CG | LEU | I | 33 | 24.751 | −38.706 | −14.345 | 1.00 | 25.79 | C |
| ATOM | 3593 | CD1 | LEU | I | 33 | 24.174 | −37.617 | −15.218 | 1.00 | 25.39 | C |
| ATOM | 3594 | CD2 | LEU | I | 33 | 24.626 | −40.072 | −15.050 | 1.00 | 25.28 | C |
| ATOM | 3595 | C | LEU | I | 33 | 28.428 | −38.832 | −12.945 | 1.00 | 21.52 | C |
| ATOM | 3596 | O | LEU | I | 33 | 28.620 | −37.777 | −12.347 | 1.00 | 20.97 | O |
| ATOM | 3597 | N | ILE | I | 34 | 29.408 | −39.580 | −13.440 | 1.00 | 20.73 | N |
| ATOM | 3598 | CA | ILE | I | 34 | 30.809 | −39.198 | −13.324 | 1.00 | 20.11 | C |
| ATOM | 3599 | CB | ILE | I | 34 | 31.710 | −40.396 | −12.936 | 1.00 | 20.28 | C |
| ATOM | 3600 | CG1 | ILE | I | 34 | 31.101 | −41.156 | −11.735 | 1.00 | 22.64 | C |
| ATOM | 3601 | CD1 | ILE | I | 34 | 31.836 | −41.069 | −10.419 | 1.00 | 26.50 | C |
| ATOM | 3602 | CG2 | ILE | I | 34 | 33.179 | −39.929 | −12.758 | 1.00 | 19.13 | C |
| ATOM | 3603 | C | ILE | I | 34 | 31.291 | −38.636 | −14.638 | 1.00 | 19.77 | C |
| ATOM | 3604 | O | ILE | I | 34 | 31.205 | −39.293 | −15.679 | 1.00 | 19.43 | O |
| ATOM | 3605 | N | GLU | I | 35 | 31.785 | −37.410 | −14.578 | 1.00 | 19.42 | N |
| ATOM | 3606 | CA | GLU | I | 35 | 32.294 | −36.727 | −15.752 | 1.00 | 20.23 | C |
| ATOM | 3607 | CB | GLU | I | 35 | 31.741 | −35.309 | −15.811 | 1.00 | 20.13 | C |
| ATOM | 3608 | CG | GLU | I | 35 | 30.240 | −35.226 | −16.025 | 1.00 | 20.20 | C |
| ATOM | 3609 | CD | GLU | I | 35 | 29.732 | −33.812 | −15.863 | 1.00 | 21.37 | C |
| ATOM | 3610 | OE1 | GLU | I | 35 | 29.189 | −33.479 | −14.785 | 1.00 | 20.71 | O |
| ATOM | 3611 | OE2 | GLU | I | 35 | 29.906 | −33.015 | −16.797 | 1.00 | 20.62 | O |
| ATOM | 3612 | C | GLU | I | 35 | 33.807 | −36.651 | −15.697 | 1.00 | 20.32 | C |
| ATOM | 3613 | O | GLU | I | 35 | 34.405 | −36.607 | −14.613 | 1.00 | 20.59 | O |
| ATOM | 3614 | N | TRP | I | 36 | 34.415 | −36.619 | −16.869 | 1.00 | 20.16 | N |
| ATOM | 3615 | CA | TRP | I | 36 | 35.832 | −36.381 | −16.972 | 1.00 | 20.14 | C |
| ATOM | 3616 | CB | TRP | I | 36 | 36.558 | −37.560 | −17.620 | 1.00 | 19.82 | C |
| ATOM | 3617 | CG | TRP | I | 36 | 36.559 | −38.801 | −16.767 | 1.00 | 21.17 | C |
| ATOM | 3618 | CD1 | TRP | I | 36 | 35.554 | −39.722 | −16.666 | 1.00 | 21.05 | C |
| ATOM | 3619 | NE1 | TRP | I | 36 | 35.923 | −40.733 | −15.800 | 1.00 | 22.29 | N |
| ATOM | 3620 | CE2 | TRP | I | 36 | 37.181 | −40.475 | −15.320 | 1.00 | 21.92 | C |
| ATOM | 3621 | CD2 | TRP | I | 36 | 37.614 | −39.260 | −15.901 | 1.00 | 22.67 | C |
| ATOM | 3622 | CE3 | TRP | I | 36 | 38.887 | −38.767 | −15.569 | 1.00 | 22.30 | C |
| ATOM | 3623 | CZ3 | TRP | I | 36 | 39.673 | −39.498 | −14.661 | 1.00 | 23.91 | C |
| ATOM | 3624 | CH2 | TRP | I | 36 | 39.205 | −40.702 | −14.098 | 1.00 | 21.74 | C |
| ATOM | 3625 | CZ2 | TRP | I | 36 | 37.971 | −41.203 | −14.411 | 1.00 | 21.98 | C |
| ATOM | 3626 | C | TRP | I | 36 | 36.022 | −35.111 | −17.762 | 1.00 | 20.18 | C |
| ATOM | 3627 | O | TRP | I | 36 | 35.345 | −34.887 | −18.772 | 1.00 | 19.42 | O |
| ATOM | 3628 | N | ILE | I | 37 | 36.924 | −34.276 | −17.252 | 1.00 | 20.39 | N |
| ATOM | 3629 | CA | ILE | I | 37 | 37.205 | −32.937 | −17.795 | 1.00 | 21.33 | C |
| ATOM | 3630 | CB | ILE | I | 37 | 36.450 | −31.820 | −17.009 | 1.00 | 21.25 | C |
| ATOM | 3631 | CG1 | ILE | I | 37 | 34.937 | −31.989 | −17.144 | 1.00 | 21.12 | C |
| ATOM | 3632 | CD1 | ILE | I | 37 | 34.134 | −31.401 | −15.972 | 1.00 | 21.98 | C |
| ATOM | 3633 | CG2 | ILE | I | 37 | 36.861 | −30.412 | −17.515 | 1.00 | 22.55 | C |
| ATOM | 3634 | C | ILE | I | 37 | 38.704 | −32.688 | −17.692 | 1.00 | 21.32 | C |
| ATOM | 3635 | O | ILE | I | 37 | 39.306 | −32.890 | −16.630 | 1.00 | 21.90 | O |
| ATOM | 3636 | N | ARG | I | 38 | 39.313 | −32.281 | −18.797 | 1.00 | 21.59 | N |
| ATOM | 3637 | CA | ARG | I | 38 | 40.737 | −32.032 | −18.787 | 1.00 | 21.91 | C |
| ATOM | 3638 | CB | ARG | I | 38 | 41.487 | −32.887 | −19.818 | 1.00 | 21.49 | C |
| ATOM | 3639 | CG | ARG | I | 38 | 41.374 | −32.416 | −21.254 | 1.00 | 21.18 | C |
| ATOM | 3640 | CD | ARG | I | 38 | 42.269 | −33.269 | −22.116 | 1.00 | 20.31 | C |
| ATOM | 3641 | NE | ARG | I | 38 | 42.243 | −32.858 | −23.515 | 1.00 | 21.99 | N |
| ATOM | 3642 | CZ | ARG | I | 38 | 42.818 | −33.543 | −24.503 | 1.00 | 22.14 | C |
| ATOM | 3643 | NH1 | ARG | I | 38 | 43.456 | −34.686 | −24.247 | 1.00 | 21.44 | N |
| ATOM | 3644 | NH2 | ARG | I | 38 | 42.745 | −33.095 | −25.750 | 1.00 | 20.81 | N |
| ATOM | 3645 | C | ARG | I | 38 | 40.996 | −30.552 | −18.993 | 1.00 | 22.56 | C |
| ATOM | 3646 | O | ARG | I | 38 | 40.185 | −29.827 | −19.584 | 1.00 | 21.48 | O |
| ATOM | 3647 | N | GLN | I | 39 | 42.129 | −30.110 | −18.469 | 1.00 | 24.18 | N |
| ATOM | 3648 | CA | GLN | I | 39 | 42.476 | −28.696 | −18.536 | 1.00 | 26.36 | C |
| ATOM | 3649 | CB | GLN | I | 39 | 42.273 | −28.018 | −17.191 | 1.00 | 25.18 | C |
| ATOM | 3650 | CG | GLN | I | 39 | 42.482 | −26.498 | −17.232 | 1.00 | 25.68 | C |
| ATOM | 3651 | CD | GLN | I | 39 | 41.980 | −25.816 | −15.971 | 1.00 | 25.46 | C |
| ATOM | 3652 | OE1 | GLN | I | 39 | 42.132 | −26.343 | −14.857 | 1.00 | 24.19 | O |
| ATOM | 3653 | NE2 | GLN | I | 39 | 41.380 | −24.635 | −16.136 | 1.00 | 22.90 | N |
| ATOM | 3654 | C | GLN | I | 39 | 43.912 | −28.579 | −18.962 | 1.00 | 28.30 | C |
| ATOM | 3655 | O | GLN | I | 39 | 44.823 | −29.048 | −18.268 | 1.00 | 28.13 | O |
| ATOM | 3656 | N | MET | I | 40 | 44.079 | −27.943 | −20.107 | 1.00 | 31.43 | N |
| ATOM | 3657 | CA | MET | I | 40 | 45.364 | −27.759 | −20.743 | 1.00 | 34.78 | C |
| ATOM | 3658 | CB | MET | I | 40 | 45.139 | −27.428 | −22.229 | 1.00 | 35.65 | C |
| ATOM | 3659 | CG | MET | I | 40 | 43.757 | −27.870 | −22.777 | 1.00 | 37.46 | C |
| ATOM | 3660 | SD | MET | I | 40 | 43.542 | −29.650 | −23.109 | 1.00 | 42.07 | S |
| ATOM | 3661 | CE | MET | I | 40 | 44.270 | −29.749 | −24.750 | 1.00 | 39.58 | C |
| ATOM | 3662 | C | MET | I | 40 | 46.120 | −26.632 | −20.019 | 1.00 | 36.34 | C |
| ATOM | 3663 | O | MET | I | 40 | 45.490 | −25.747 | −19.420 | 1.00 | 35.96 | O |
| ATOM | 3664 | N | PRO | I | 41 | 47.472 | −26.661 | −20.051 | 1.00 | 38.01 | N |
| ATOM | 3665 | CA | PRO | I | 41 | 48.257 | −25.596 | −19.385 | 1.00 | 38.87 | C |
| ATOM | 3666 | CB | PRO | I | 41 | 49.654 | −25.728 | −20.026 | 1.00 | 39.48 | C |
| ATOM | 3667 | CG | PRO | I | 41 | 49.481 | −26.708 | −21.216 | 1.00 | 39.29 | C |
| ATOM | 3668 | CD | PRO | I | 41 | 48.340 | −27.593 | −20.796 | 1.00 | 38.40 | C |
| ATOM | 3669 | C | PRO | I | 41 | 47.674 | −24.182 | −19.625 | 1.00 | 39.24 | C |
| ATOM | 3670 | O | PRO | I | 41 | 47.522 | −23.762 | −20.787 | 1.00 | 39.75 | O |
| ATOM | 3671 | N | GLY | I | 42 | 47.315 | −23.492 | −18.538 | 1.00 | 39.14 | N |
| ATOM | 3672 | CA | GLY | I | 42 | 46.686 | −22.152 | −18.586 | 1.00 | 38.85 | C |
| ATOM | 3673 | C | GLY | I | 42 | 45.463 | −21.952 | −19.486 | 1.00 | 38.61 | C |
| ATOM | 3674 | O | GLY | I | 42 | 45.126 | −20.815 | −19.866 | 1.00 | 38.43 | O |
| ATOM | 3675 | N | GLN | I | 43 | 44.777 | −23.043 | −19.811 | 1.00 | 37.58 | N |
| ATOM | 3676 | CA | GLN | I | 43 | 43.693 | −22.997 | −20.791 | 1.00 | 36.84 | C |
| ATOM | 3677 | CB | GLN | I | 43 | 44.002 | −23.950 | −21.957 | 1.00 | 37.42 | C |
| ATOM | 3678 | CG | GLN | I | 43 | 45.156 | −23.509 | −22.857 | 1.00 | 40.27 | C |
| ATOM | 3679 | CD | GLN | I | 43 | 44.704 | −22.605 | −23.995 | 1.00 | 44.22 | C |
| ATOM | 3680 | OE1 | GLN | I | 43 | 43.509 | −22.347 | −24.167 | 1.00 | 46.23 | O |
| ATOM | 3681 | NE2 | GLN | I | 43 | 45.663 | −22.121 | −24.785 | 1.00 | 44.73 | N |
| ATOM | 3682 | C | GLN | I | 43 | 42.292 | −23.269 | −20.197 | 1.00 | 35.15 | C |
| ATOM | 3683 | O | GLN | I | 43 | 42.096 | −23.256 | −18.971 | 1.00 | 34.61 | O |
| ATOM | 3684 | N | GLY | I | 44 | 41.320 | −23.477 | −21.082 | 1.00 | 33.42 | N |
| ATOM | 3685 | CA | GLY | I | 44 | 39.946 | −23.719 | −20.683 | 1.00 | 31.61 | C |
| ATOM | 3686 | C | GLY | I | 44 | 39.688 | −25.173 | −20.318 | 1.00 | 29.92 | C |
| ATOM | 3687 | O | GLY | I | 44 | 40.605 | −25.991 | −20.256 | 1.00 | 29.16 | O |
| ATOM | 3688 | N | LEU | I | 45 | 38.423 | −25.483 | −20.067 | 1.00 | 29.16 | N |
| ATOM | 3689 | CA | LEU | I | 45 | 38.018 | −26.820 | −19.667 | 1.00 | 28.27 | C |
| ATOM | 3690 | CB | LEU | I | 45 | 36.951 | −26.736 | −18.575 | 1.00 | 28.43 | C |
| ATOM | 3691 | CG | LEU | I | 45 | 37.408 | −26.005 | −17.312 | 1.00 | 28.28 | C |
| ATOM | 3692 | CD1 | LEU | I | 45 | 36.239 | −25.759 | −16.371 | 1.00 | 28.88 | C |
| ATOM | 3693 | CD2 | LEU | I | 45 | 38.535 | −26.779 | −16.614 | 1.00 | 28.72 | C |
| ATOM | 3694 | C | LEU | I | 45 | 37.502 | −27.602 | −20.871 | 1.00 | 28.13 | C |
| ATOM | 3695 | O | LEU | I | 45 | 36.602 | −27.129 | −21.581 | 1.00 | 28.50 | O |
| ATOM | 3696 | N | GLU | I | 46 | 38.078 | −28.785 | −21.109 | 1.00 | 26.17 | N |
| ATOM | 3697 | CA | GLU | I | 46 | 37.624 | −29.622 | −22.217 | 1.00 | 25.14 | C |
| ATOM | 3698 | CB | GLU | I | 46 | 38.790 | −30.128 | −23.082 | 1.00 | 24.94 | C |
| ATOM | 3699 | CG | GLU | I | 46 | 39.416 | −29.098 | −24.006 | 1.00 | 29.34 | C |
| ATOM | 3700 | CD | GLU | I | 46 | 40.248 | −29.738 | −25.132 | 1.00 | 33.23 | C |
| ATOM | 3701 | OE1 | GLU | I | 46 | 40.638 | −30.929 | −25.025 | 1.00 | 33.72 | O |
| ATOM | 3702 | OE2 | GLU | I | 46 | 40.525 | −29.033 | −26.123 | 1.00 | 36.55 | O |
| ATOM | 3703 | C | GLU | I | 46 | 36.882 | −30.808 | −21.638 | 1.00 | 23.16 | C |
| ATOM | 3704 | O | GLU | I | 46 | 37.485 | −31.647 | −20.964 | 1.00 | 22.25 | O |
| ATOM | 3705 | N | TRP | I | 47 | 35.584 | −30.861 | −21.905 | 1.00 | 21.83 | N |
| ATOM | 3706 | CA | TRP | I | 47 | 34.720 | −31.942 | −21.390 | 1.00 | 21.59 | C |
| ATOM | 3707 | CB | TRP | I | 47 | 33.257 | −31.507 | −21.491 | 1.00 | 21.55 | C |
| ATOM | 3708 | CG | TRP | I | 47 | 32.272 | −32.418 | −20.841 | 1.00 | 21.21 | C |
| ATOM | 3709 | CD1 | TRP | I | 47 | 31.953 | −32.472 | −19.510 | 1.00 | 21.19 | C |
| ATOM | 3710 | NE1 | TRP | I | 47 | 30.985 | −33.426 | −19.298 | 1.00 | 19.85 | N |
| ATOM | 3711 | CE2 | TRP | I | 47 | 30.651 | −33.996 | −20.500 | 1.00 | 20.54 | C |
| ATOM | 3712 | CD2 | TRP | I | 47 | 31.445 | −33.382 | −21.495 | 1.00 | 20.90 | C |
| ATOM | 3713 | CE3 | TRP | I | 47 | 31.289 | −33.787 | −22.835 | 1.00 | 20.68 | C |
| ATOM | 3714 | CZ3 | TRP | I | 47 | 30.365 | −34.792 | −23.132 | 1.00 | 21.04 | C |
| ATOM | 3715 | CH2 | TRP | I | 47 | 29.590 | −35.386 | −22.119 | 1.00 | 22.01 | C |
| ATOM | 3716 | CZ2 | TRP | I | 47 | 29.720 | −35.004 | −20.799 | 1.00 | 21.06 | C |
| ATOM | 3717 | C | TRP | I | 47 | 34.962 | −33.198 | −22.227 | 1.00 | 20.95 | C |
| ATOM | 3718 | O | TRP | I | 47 | 34.911 | −33.147 | −23.456 | 1.00 | 20.93 | O |
| ATOM | 3719 | N | ILE | I | 48 | 35.281 | −34.309 | −21.564 | 1.00 | 20.12 | N |
| ATOM | 3720 | CA | ILE | I | 48 | 35.593 | −35.556 | −22.262 | 1.00 | 19.27 | C |
| ATOM | 3721 | CB | ILE | I | 48 | 36.749 | −36.324 | −21.546 | 1.00 | 19.53 | C |
| ATOM | 3722 | CG1 | ILE | I | 48 | 38.062 | −35.513 | −21.639 | 1.00 | 18.36 | C |
| ATOM | 3723 | CD1 | ILE | I | 48 | 39.074 | −35.852 | −20.533 | 1.00 | 19.75 | C |
| ATOM | 3724 | CG2 | ILE | I | 48 | 36.936 | −37.702 | −22.153 | 1.00 | 19.47 | C |
| ATOM | 3725 | C | ILE | I | 48 | 34.369 | −36.443 | −22.407 | 1.00 | 19.06 | C |
| ATOM | 3726 | O | ILE | I | 48 | 34.118 | −36.994 | −23.466 | 1.00 | 19.16 | O |
| ATOM | 3727 | N | GLY | I | 49 | 33.615 | −36.606 | −21.332 | 1.00 | 19.18 | N |
| ATOM | 3728 | CA | GLY | I | 49 | 32.503 | −37.557 | −21.367 | 1.00 | 18.75 | C |
| ATOM | 3729 | C | GLY | I | 49 | 31.908 | −37.784 | −19.999 | 1.00 | 19.16 | C |
| ATOM | 3730 | O | GLY | I | 49 | 32.386 | −37.230 | −19.001 | 1.00 | 19.20 | O |
| ATOM | 3731 | N | LEU | I | 50 | 30.850 | −38.583 | −19.969 | 1.00 | 19.28 | N |
| ATOM | 3732 | CA | LEU | I | 50 | 30.073 | −38.831 | −18.767 | 1.00 | 20.30 | C |
| ATOM | 3733 | CB | LEU | I | 50 | 28.794 | −37.974 | −18.802 | 1.00 | 20.57 | C |
| ATOM | 3734 | CG | LEU | I | 50 | 27.584 | −38.169 | −17.891 | 1.00 | 24.57 | C |
| ATOM | 3735 | CD1 | LEU | I | 50 | 26.777 | −36.860 | −17.860 | 1.00 | 24.58 | C |
| ATOM | 3736 | CD2 | LEU | I | 50 | 26.693 | −39.357 | −18.343 | 1.00 | 22.48 | C |
| ATOM | 3737 | C | LEU | I | 50 | 29.748 | −40.329 | −18.742 | 1.00 | 19.39 | C |
| ATOM | 3738 | O | LEU | I | 50 | 29.510 | −40.943 | −19.792 | 1.00 | 18.14 | O |
| ATOM | 3739 | N | ILE | I | 51 | 29.762 | −40.904 | −17.546 | 1.00 | 18.11 | N |
| ATOM | 3740 | CA | ILE | I | 51 | 29.349 | −42.278 | −17.349 | 1.00 | 17.76 | C |
| ATOM | 3741 | CB | ILE | I | 51 | 30.563 | −43.262 | −17.266 | 1.00 | 17.83 | C |
| ATOM | 3742 | CG1 | ILE | I | 51 | 30.068 | −44.706 | −17.235 | 1.00 | 17.84 | C |
| ATOM | 3743 | CD1 | ILE | I | 51 | 31.135 | −45.760 | −17.458 | 1.00 | 17.67 | C |
| ATOM | 3744 | CG2 | ILE | I | 51 | 31.500 | −42.943 | −16.072 | 1.00 | 17.56 | C |
| ATOM | 3745 | C | ILE | I | 51 | 28.452 | −42.386 | −16.107 | 1.00 | 18.14 | C |
| ATOM | 3746 | O | ILE | I | 51 | 28.714 | −41.728 | −15.085 | 1.00 | 16.97 | O |
| ATOM | 3747 | N | ASN | I | 52 | 27.389 | −43.186 | −16.228 | 1.00 | 18.14 | N |
| ATOM | 3748 | CA | ASN | I | 52 | 26.621 | −43.656 | −15.067 | 1.00 | 18.47 | C |
| ATOM | 3749 | CB | ASN | I | 52 | 25.115 | −43.678 | −15.361 | 1.00 | 18.42 | C |
| ATOM | 3750 | CG | ASN | I | 52 | 24.320 | −44.290 | −14.211 | 1.00 | 18.65 | C |
| ATOM | 3751 | OD1 | ASN | I | 52 | 24.534 | −43.947 | −13.048 | 1.00 | 21.14 | O |
| ATOM | 3752 | ND2 | ASN | I | 52 | 23.446 | −45.217 | −14.523 | 1.00 | 15.59 | N |
| ATOM | 3753 | C | ASN | I | 52 | 27.073 | −45.076 | −14.755 | 1.00 | 18.94 | C |
| ATOM | 3754 | O | ASN | I | 52 | 26.784 | −45.988 | −15.532 | 1.00 | 18.75 | O |
| ATOM | 3755 | N | PRO | I | 52A | 27.783 | −45.279 | −13.631 | 1.00 | 19.40 | N |
| ATOM | 3756 | CA | PRO | I | 52A | 28.348 | −46.611 | −13.334 | 1.00 | 20.68 | C |
| ATOM | 3757 | CB | PRO | I | 52A | 28.928 | −46.425 | −11.927 | 1.00 | 19.84 | C |
| ATOM | 3758 | CG | PRO | I | 52A | 29.371 | −45.034 | −11.942 | 1.00 | 20.62 | C |
| ATOM | 3759 | CD | PRO | I | 52A | 28.221 | −44.299 | −12.629 | 1.00 | 19.38 | C |
| ATOM | 3760 | C | PRO | I | 52A | 27.418 | −47.836 | −13.402 | 1.00 | 21.70 | C |
| ATOM | 3761 | O | PRO | I | 52A | 27.782 | −48.844 | −14.019 | 1.00 | 24.23 | O |
| ATOM | 3762 | N | GLY | I | 53 | 26.228 | −47.811 | −12.830 | 1.00 | 21.91 | N |
| ATOM | 3763 | CA | GLY | I | 53 | 25.480 | −49.119 | −12.868 | 1.00 | 20.50 | C |
| ATOM | 3764 | C | GLY | I | 53 | 25.015 | −49.587 | −14.250 | 1.00 | 19.49 | C |
| ATOM | 3765 | O | GLY | I | 53 | 25.204 | −50.751 | −14.659 | 1.00 | 19.31 | O |
| ATOM | 3766 | N | SER | I | 54 | 24.394 | −48.671 | −14.968 | 1.00 | 18.73 | N |
| ATOM | 3767 | CA | SER | I | 54 | 23.894 | −48.901 | −16.310 | 1.00 | 18.08 | C |
| ATOM | 3768 | CB | SER | I | 54 | 22.950 | −47.752 | −16.661 | 1.00 | 18.35 | C |
| ATOM | 3769 | OG | SER | I | 54 | 23.652 | −46.504 | −16.718 | 1.00 | 19.50 | O |
| ATOM | 3770 | C | SER | I | 54 | 25.012 | −48.924 | −17.355 | 1.00 | 18.32 | C |
| ATOM | 3771 | O | SER | I | 54 | 24.799 | −49.367 | −18.481 | 1.00 | 17.06 | O |
| ATOM | 3772 | N | ASP | I | 55 | 26.181 | −48.405 | −16.969 | 1.00 | 17.97 | N |
| ATOM | 3773 | CA | ASP | I | 55 | 27.277 | −48.112 | −17.886 | 1.00 | 18.77 | C |
| ATOM | 3774 | CB | ASP | I | 55 | 27.940 | −49.399 | −18.385 | 1.00 | 18.98 | C |
| ATOM | 3775 | CG | ASP | I | 55 | 28.922 | −49.980 | −17.375 | 1.00 | 22.62 | C |
| ATOM | 3776 | OD1 | ASP | I | 55 | 29.300 | −51.154 | −17.545 | 1.00 | 23.80 | O |
| ATOM | 3777 | OD2 | ASP | I | 55 | 29.309 | −49.269 | −16.410 | 1.00 | 25.26 | O |
| ATOM | 3778 | C | ASP | I | 55 | 26.868 | −47.196 | −19.054 | 1.00 | 18.02 | C |
| ATOM | 3779 | O | ASP | I | 55 | 27.457 | −47.259 | −20.129 | 1.00 | 17.96 | O |
| ATOM | 3780 | N | TYR | I | 56 | 25.844 | −46.374 | −18.847 | 1.00 | 18.61 | N |
| ATOM | 3781 | CA | TYR | I | 56 | 25.451 | −45.394 | −19.845 | 1.00 | 18.69 | C |
| ATOM | 3782 | CB | TYR | I | 56 | 24.185 | −44.642 | −19.438 | 1.00 | 19.47 | C |
| ATOM | 3783 | CG | TYR | I | 56 | 23.849 | −43.469 | −20.345 | 1.00 | 21.09 | C |
| ATOM | 3784 | CD1 | TYR | I | 56 | 22.970 | −43.616 | −21.420 | 1.00 | 24.58 | C |
| ATOM | 3785 | CE1 | TYR | I | 56 | 22.668 | −42.530 | −22.260 | 1.00 | 24.45 | C |
| ATOM | 3786 | CZ | TYR | I | 56 | 23.249 | −41.298 | −22.008 | 1.00 | 25.95 | C |
| ATOM | 3787 | OH | TYR | I | 56 | 22.973 | −40.214 | −22.804 | 1.00 | 27.56 | O |
| ATOM | 3788 | CE2 | TYR | I | 56 | 24.118 | −41.137 | −20.943 | 1.00 | 25.07 | C |
| ATOM | 3789 | CD2 | TYR | I | 56 | 24.409 | −42.223 | −20.124 | 1.00 | 21.53 | C |
| ATOM | 3790 | C | TYR | I | 56 | 26.620 | −44.413 | −19.984 | 1.00 | 18.93 | C |
| ATOM | 3791 | O | TYR | I | 56 | 27.141 | −43.913 | −18.986 | 1.00 | 17.94 | O |
| ATOM | 3792 | N | THR | I | 57 | 27.058 | −44.185 | −21.217 | 1.00 | 18.47 | N |
| ATOM | 3793 | CA | THR | I | 57 | 28.101 | −43.180 | −21.455 | 1.00 | 19.70 | C |
| ATOM | 3794 | CB | THR | I | 57 | 29.434 | −43.798 | −21.917 | 1.00 | 19.23 | C |
| ATOM | 3795 | OG1 | THR | I | 57 | 29.194 | −44.638 | −23.042 | 1.00 | 20.54 | O |
| ATOM | 3796 | CG2 | THR | I | 57 | 30.059 | −44.628 | −20.827 | 1.00 | 18.95 | C |
| ATOM | 3797 | C | THR | I | 57 | 27.670 | −42.179 | −22.507 | 1.00 | 20.26 | C |
| ATOM | 3798 | O | THR | I | 57 | 26.803 | −42.449 | −23.338 | 1.00 | 20.28 | O |
| ATOM | 3799 | N | ASN | I | 58 | 28.285 | −41.013 | −22.445 | 1.00 | 20.52 | N |
| ATOM | 3800 | CA | ASN | I | 58 | 28.160 | −40.018 | −23.473 | 1.00 | 20.84 | C |
| ATOM | 3801 | CB | ASN | I | 58 | 27.077 | −38.993 | −23.133 | 1.00 | 21.25 | C |
| ATOM | 3802 | CG | ASN | I | 58 | 26.811 | −38.034 | −24.289 | 1.00 | 22.19 | C |
| ATOM | 3803 | OD1 | ASN | I | 58 | 27.612 | −37.161 | −24.561 | 1.00 | 25.74 | O |
| ATOM | 3804 | ND2 | ASN | I | 58 | 25.684 | −38.200 | −24.963 | 1.00 | 22.73 | N |
| ATOM | 3805 | C | ASN | I | 58 | 29.523 | −39.357 | −23.616 | 1.00 | 20.82 | C |
| ATOM | 3806 | O | ASN | I | 58 | 30.099 | −38.887 | −22.623 | 1.00 | 20.17 | O |
| ATOM | 3807 | N | TYR | I | 59 | 30.041 | −39.364 | −24.844 | 1.00 | 20.43 | N |
| ATOM | 3808 | CA | TYR | I | 59 | 31.398 | −38.908 | −25.142 | 1.00 | 20.76 | C |
| ATOM | 3809 | CB | TYR | I | 59 | 32.118 | −39.964 | −25.991 | 1.00 | 20.71 | C |
| ATOM | 3810 | CG | TYR | I | 59 | 32.551 | −41.191 | −25.247 | 1.00 | 21.02 | C |
| ATOM | 3811 | CD1 | TYR | I | 59 | 31.719 | −42.300 | −25.169 | 1.00 | 21.33 | C |
| ATOM | 3812 | CE1 | TYR | I | 59 | 32.104 | −43.440 | −24.494 | 1.00 | 20.89 | C |
| ATOM | 3813 | CZ | TYR | I | 59 | 33.337 | −43.486 | −23.879 | 1.00 | 20.81 | C |
| ATOM | 3814 | OH | TYR | I | 59 | 33.691 | −44.633 | −23.217 | 1.00 | 21.31 | O |
| ATOM | 3815 | CE2 | TYR | I | 59 | 34.206 | −42.396 | −23.932 | 1.00 | 21.31 | C |
| ATOM | 3816 | CD2 | TYR | I | 59 | 33.805 | −41.246 | −24.618 | 1.00 | 21.25 | C |
| ATOM | 3817 | C | TYR | I | 59 | 31.402 | −37.622 | −25.947 | 1.00 | 20.39 | C |
| ATOM | 3818 | O | TYR | I | 59 | 30.582 | −37.450 | −26.847 | 1.00 | 20.36 | O |
| ATOM | 3819 | N | ASN | I | 60 | 32.348 | −36.734 | −25.657 | 1.00 | 20.12 | N |
| ATOM | 3820 | CA | ASN | I | 60 | 32.694 | −35.683 | −26.612 | 1.00 | 19.84 | C |
| ATOM | 3821 | CB | ASN | I | 60 | 33.774 | −34.759 | −26.029 | 1.00 | 19.62 | C |
| ATOM | 3822 | CG | ASN | I | 60 | 34.227 | −33.654 | −26.992 | 1.00 | 20.10 | C |
| ATOM | 3823 | OD1 | ASN | I | 60 | 34.083 | −33.739 | −28.235 | 1.00 | 16.22 | O |
| ATOM | 3824 | ND2 | ASN | I | 60 | 34.819 | −32.616 | −26.417 | 1.00 | 17.78 | N |
| ATOM | 3825 | C | ASN | I | 60 | 33.202 | −36.371 | −27.883 | 1.00 | 20.17 | C |
| ATOM | 3826 | O | ASN | I | 60 | 34.186 | −37.129 | −27.835 | 1.00 | 19.09 | O |
| ATOM | 3827 | N | GLU | I | 61 | 32.496 | −36.118 | −28.985 | 1.00 | 19.83 | N |
| ATOM | 3828 | CA | GLU | I | 61 | 32.821 | −36.622 | −30.331 | 1.00 | 22.20 | C |
| ATOM | 3829 | CB | GLU | I | 61 | 32.027 | −35.801 | −31.371 | 1.00 | 22.31 | C |
| ATOM | 3830 | CG | GLU | I | 61 | 31.805 | −36.440 | −32.735 | 1.00 | 27.32 | C |
| ATOM | 3831 | CD | GLU | I | 61 | 31.408 | −35.403 | −33.834 | 1.00 | 30.51 | C |
| ATOM | 3832 | OE1 | GLU | I | 61 | 30.954 | −35.827 | −34.930 | 1.00 | 30.28 | O |
| ATOM | 3833 | OE2 | GLU | I | 61 | 31.551 | −34.171 | −33.592 | 1.00 | 32.98 | O |
| ATOM | 3834 | C | GLU | I | 61 | 34.328 | −36.517 | −30.639 | 1.00 | 21.37 | C |
| ATOM | 3835 | O | GLU | I | 61 | 34.916 | −37.424 | −31.220 | 1.00 | 21.62 | O |
| ATOM | 3836 | N | ASN | I | 62 | 34.950 | −35.416 | −30.247 | 1.00 | 21.55 | N |
| ATOM | 3837 | CA | ASN | I | 62 | 36.373 | −35.217 | −30.523 | 1.00 | 22.66 | C |
| ATOM | 3838 | CB | ASN | I | 62 | 36.824 | −33.844 | −30.052 | 1.00 | 23.21 | C |
| ATOM | 3839 | CG | ASN | I | 62 | 36.086 | −32.715 | −30.757 | 1.00 | 25.98 | C |
| ATOM | 3840 | OD1 | ASN | I | 62 | 35.412 | −32.931 | −31.764 | 1.00 | 30.03 | O |
| ATOM | 3841 | ND2 | ASN | I | 62 | 36.202 | −31.512 | −30.222 | 1.00 | 26.36 | N |
| ATOM | 3842 | C | ASN | I | 62 | 37.301 | −36.300 | −29.950 | 1.00 | 23.02 | C |
| ATOM | 3843 | O | ASN | I | 62 | 38.372 | −36.539 | −30.497 | 1.00 | 22.88 | O |
| ATOM | 3844 | N | PHE | I | 63 | 36.882 | −36.953 | −28.864 | 1.00 | 22.58 | N |
| ATOM | 3845 | CA | PHE | I | 63 | 37.758 | −37.882 | −28.146 | 1.00 | 23.23 | C |
| ATOM | 3846 | CB | PHE | I | 63 | 37.924 | −37.422 | −26.687 | 1.00 | 23.69 | C |
| ATOM | 3847 | CG | PHE | I | 63 | 38.357 | −35.985 | −26.556 | 1.00 | 25.04 | C |
| ATOM | 3848 | CD1 | PHE | I | 63 | 37.630 | −35.097 | −25.774 | 1.00 | 24.76 | C |
| ATOM | 3849 | CE1 | PHE | I | 63 | 38.026 | −33.757 | −25.658 | 1.00 | 26.08 | C |
| ATOM | 3850 | CZ | PHE | I | 63 | 39.144 | −33.301 | −26.333 | 1.00 | 26.80 | C |
| ATOM | 3851 | CE2 | PHE | I | 63 | 39.877 | −34.181 | −27.144 | 1.00 | 28.15 | C |
| ATOM | 3852 | CD2 | PHE | I | 63 | 39.478 | −35.517 | −27.246 | 1.00 | 26.84 | C |
| ATOM | 3853 | C | PHE | I | 63 | 37.296 | −39.334 | −28.203 | 1.00 | 23.22 | C |
| ATOM | 3854 | O | PHE | I | 63 | 37.938 | −40.221 | −27.631 | 1.00 | 22.85 | O |
| ATOM | 3855 | N | LYS | I | 64 | 36.199 | −39.572 | −28.919 | 1.00 | 23.09 | N |
| ATOM | 3856 | CA | LYS | I | 64 | 35.552 | −40.883 | −28.954 | 1.00 | 23.72 | C |
| ATOM | 3857 | CB | LYS | I | 64 | 34.289 | −40.827 | −29.827 | 1.00 | 24.06 | C |
| ATOM | 3858 | CG | LYS | I | 64 | 33.487 | −42.136 | −29.900 | 1.00 | 26.19 | C |
| ATOM | 3859 | CD | LYS | I | 64 | 32.199 | −42.035 | −29.102 | 1.00 | 30.15 | C |
| ATOM | 3860 | CE | LYS | I | 64 | 31.454 | −43.375 | −29.050 | 1.00 | 31.18 | C |
| ATOM | 3861 | NZ | LYS | I | 64 | 31.986 | −44.263 | −27.961 | 1.00 | 31.32 | N |
| ATOM | 3862 | C | LYS | I | 64 | 36.486 | −42.020 | −29.411 | 1.00 | 23.49 | C |
| ATOM | 3863 | O | LYS | I | 64 | 36.422 | −43.127 | −28.871 | 1.00 | 23.92 | O |
| ATOM | 3864 | N | GLY | I | 65 | 37.340 | −41.745 | −30.395 | 1.00 | 23.06 | N |
| ATOM | 3865 | CA | GLY | I | 65 | 38.321 | −42.717 | −30.886 | 1.00 | 22.62 | C |
| ATOM | 3866 | C | GLY | I | 65 | 39.391 | −43.147 | −29.887 | 1.00 | 22.99 | C |
| ATOM | 3867 | O | GLY | I | 65 | 39.953 | −44.244 | −30.015 | 1.00 | 22.86 | O |
| ATOM | 3868 | N | GLN | I | 66 | 39.675 | −42.296 | −28.896 | 1.00 | 21.81 | N |
| ATOM | 3869 | CA | GLN | I | 66 | 40.793 | −42.539 | −27.973 | 1.00 | 22.03 | C |
| ATOM | 3870 | CB | GLN | I | 66 | 41.841 | −41.417 | −28.030 | 1.00 | 21.95 | C |
| ATOM | 3871 | CG | GLN | I | 66 | 42.540 | −41.267 | −29.385 | 1.00 | 24.43 | C |
| ATOM | 3872 | CD | GLN | I | 66 | 41.622 | −40.659 | −30.435 | 1.00 | 27.37 | C |
| ATOM | 3873 | OE1 | GLN | I | 66 | 41.484 | −41.189 | −31.540 | 1.00 | 30.61 | O |
| ATOM | 3874 | NE2 | GLN | I | 66 | 40.961 | −39.559 | −30.077 | 1.00 | 28.84 | N |
| ATOM | 3875 | C | GLN | I | 66 | 40.378 | −42.762 | −26.533 | 1.00 | 21.15 | C |
| ATOM | 3876 | O | GLN | I | 66 | 41.016 | −43.537 | −25.838 | 1.00 | 22.09 | O |
| ATOM | 3877 | N | ALA | I | 67 | 39.330 | −42.082 | −26.087 | 1.00 | 20.72 | N |
| ATOM | 3878 | CA | ALA | I | 67 | 38.900 | −42.142 | −24.684 | 1.00 | 20.68 | C |
| ATOM | 3879 | CB | ALA | I | 67 | 38.259 | −40.829 | −24.269 | 1.00 | 20.97 | C |
| ATOM | 3880 | C | ALA | I | 67 | 37.936 | −43.290 | −24.442 | 1.00 | 20.89 | C |
| ATOM | 3881 | O | ALA | I | 67 | 37.018 | −43.508 | −25.229 | 1.00 | 20.91 | O |
| ATOM | 3882 | N | THR | I | 68 | 38.151 | −44.039 | −23.363 | 1.00 | 21.21 | N |
| ATOM | 3883 | CA | THR | I | 68 | 37.195 | −45.069 | −22.939 | 1.00 | 21.23 | C |
| ATOM | 3884 | CB | THR | I | 68 | 37.739 | −46.506 | −23.158 | 1.00 | 21.79 | C |
| ATOM | 3885 | OG1 | THR | I | 68 | 38.011 | −46.732 | −24.543 | 1.00 | 21.32 | O |
| ATOM | 3886 | CG2 | THR | I | 68 | 36.717 | −47.559 | −22.700 | 1.00 | 22.56 | C |
| ATOM | 3887 | C | THR | I | 68 | 36.819 | −44.875 | −21.459 | 1.00 | 21.43 | C |
| ATOM | 3888 | O | THR | I | 68 | 37.695 | −44.893 | −20.573 | 1.00 | 21.23 | O |
| ATOM | 3889 | N | LEU | I | 69 | 35.529 | −44.659 | −21.201 | 1.00 | 20.83 | N |
| ATOM | 3890 | CA | LEU | I | 69 | 35.001 | −44.602 | −19.829 | 1.00 | 20.72 | C |
| ATOM | 3891 | CB | LEU | I | 69 | 33.847 | −43.592 | −19.740 | 1.00 | 20.91 | C |
| ATOM | 3892 | CG | LEU | I | 69 | 34.259 | −42.143 | −19.991 | 1.00 | 22.64 | C |
| ATOM | 3893 | CD1 | LEU | I | 69 | 33.089 | −41.184 | −19.791 | 1.00 | 22.94 | C |
| ATOM | 3894 | CD2 | LEU | I | 69 | 35.412 | −41.802 | −19.061 | 1.00 | 22.02 | C |
| ATOM | 3895 | C | LEU | I | 69 | 34.533 | −45.955 | −19.288 | 1.00 | 20.51 | C |
| ATOM | 3896 | O | LEU | I | 69 | 33.809 | −46.673 | −19.952 | 1.00 | 20.81 | O |
| ATOM | 3897 | N | SER | I | 70 | 34.935 | −46.298 | −18.069 | 1.00 | 20.00 | N |
| ATOM | 3898 | CA | SER | I | 70 | 34.465 | −47.534 | −17.443 | 1.00 | 19.32 | C |
| ATOM | 3899 | CB | SER | I | 70 | 35.426 | −48.696 | −17.738 | 1.00 | 20.02 | C |
| ATOM | 3900 | OG | SER | I | 70 | 36.738 | −48.401 | −17.256 | 1.00 | 19.44 | O |
| ATOM | 3901 | C | SER | I | 70 | 34.351 | −47.308 | −15.946 | 1.00 | 19.34 | C |
| ATOM | 3902 | O | SER | I | 70 | 34.832 | −46.287 | −15.420 | 1.00 | 17.34 | O |
| ATOM | 3903 | N | ALA | I | 71 | 33.706 | −48.249 | −15.268 | 1.00 | 18.93 | N |
| ATOM | 3904 | CA | ALA | I | 71 | 33.573 | −48.190 | −13.829 | 1.00 | 20.64 | C |
| ATOM | 3905 | CB | ALA | I | 71 | 32.302 | −47.434 | −13.435 | 1.00 | 20.46 | C |
| ATOM | 3906 | C | ALA | I | 71 | 33.568 | −49.603 | −13.249 | 1.00 | 21.57 | C |
| ATOM | 3907 | O | ALA | I | 71 | 33.067 | −50.541 | −13.868 | 1.00 | 21.85 | O |
| ATOM | 3908 | N | ASP | I | 72 | 34.136 | −49.743 | −12.057 | 1.00 | 22.80 | N |
| ATOM | 3909 | CA | ASP | I | 72 | 34.136 | −51.010 | −11.338 | 1.00 | 23.89 | C |
| ATOM | 3910 | CB | ASP | I | 72 | 35.573 | −51.399 | −10.949 | 1.00 | 24.21 | C |
| ATOM | 3911 | CG | ASP | I | 72 | 35.663 | −52.733 | −10.204 | 1.00 | 25.39 | C |
| ATOM | 3912 | OD1 | ASP | I | 72 | 34.635 | −53.263 | −9.719 | 1.00 | 27.47 | O |
| ATOM | 3913 | OD2 | ASP | I | 72 | 36.792 | −53.254 | −10.090 | 1.00 | 26.90 | O |
| ATOM | 3914 | C | ASP | I | 72 | 33.282 | −50.810 | −10.100 | 1.00 | 24.62 | C |
| ATOM | 3915 | O | ASP | I | 72 | 33.706 | −50.130 | −9.159 | 1.00 | 23.86 | O |
| ATOM | 3916 | N | LYS | I | 73 | 32.089 | −51.409 | −10.107 | 1.00 | 25.39 | N |
| ATOM | 3917 | CA | LYS | I | 73 | 31.152 | −51.337 | −8.971 | 1.00 | 26.84 | C |
| ATOM | 3918 | CB | LYS | I | 73 | 29.820 | −52.041 | −9.314 | 1.00 | 27.31 | C |
| ATOM | 3919 | CG | LYS | I | 73 | 29.084 | −51.440 | −10.527 | 1.00 | 29.00 | C |
| ATOM | 3920 | CD | LYS | I | 73 | 28.183 | −52.472 | −11.260 | 1.00 | 30.67 | C |
| ATOM | 3921 | CE | LYS | I | 73 | 27.897 | −52.022 | −12.723 | 1.00 | 32.50 | C |
| ATOM | 3922 | NZ | LYS | I | 73 | 26.889 | −52.854 | −13.493 | 1.00 | 29.04 | N |
| ATOM | 3923 | C | LYS | I | 73 | 31.750 | −51.922 | −7.679 | 1.00 | 27.10 | C |
| ATOM | 3924 | O | LYS | I | 73 | 31.434 | −51.457 | −6.576 | 1.00 | 27.69 | O |
| ATOM | 3925 | N | SER | I | 74 | 32.623 | −52.921 | −7.808 | 1.00 | 26.86 | N |
| ATOM | 3926 | CA | SER | I | 74 | 33.121 | −53.639 | −6.632 | 1.00 | 26.51 | C |
| ATOM | 3927 | CB | SER | I | 74 | 33.878 | −54.920 | −7.021 | 1.00 | 26.92 | C |
| ATOM | 3928 | OG | SER | I | 74 | 35.163 | −54.623 | −7.563 | 1.00 | 27.47 | O |
| ATOM | 3929 | C | SER | I | 74 | 33.997 | −52.749 | −5.766 | 1.00 | 26.13 | C |
| ATOM | 3930 | O | SER | I | 74 | 34.017 | −52.894 | −4.546 | 1.00 | 26.30 | O |
| ATOM | 3931 | N | SER | I | 75 | 34.723 | −51.833 | −6.400 | 1.00 | 24.96 | N |
| ATOM | 3932 | CA | SER | I | 75 | 35.626 | −50.951 | −5.678 | 1.00 | 24.17 | C |
| ATOM | 3933 | CB | SER | I | 75 | 37.045 | −51.103 | −6.222 | 1.00 | 24.35 | C |
| ATOM | 3934 | OG | SER | I | 75 | 37.115 | −50.603 | −7.551 | 1.00 | 23.66 | O |
| ATOM | 3935 | C | SER | I | 75 | 35.181 | −49.482 | −5.746 | 1.00 | 23.28 | C |
| ATOM | 3936 | O | SER | I | 75 | 35.883 | −48.597 | −5.256 | 1.00 | 23.27 | O |
| ATOM | 3937 | N | SER | I | 76 | 34.022 | −49.231 | −6.357 | 1.00 | 22.21 | N |
| ATOM | 3938 | CA | SER | I | 76 | 33.517 | −47.865 | −6.577 | 1.00 | 21.44 | C |
| ATOM | 3939 | CB | SER | I | 76 | 33.095 | −47.214 | −5.255 | 1.00 | 21.55 | C |
| ATOM | 3940 | OG | SER | I | 76 | 31.907 | −47.804 | −4.791 | 1.00 | 21.46 | O |
| ATOM | 3941 | C | SER | I | 76 | 34.534 | −46.987 | −7.306 | 1.00 | 21.19 | C |
| ATOM | 3942 | O | SER | I | 76 | 34.687 | −45.805 | −6.995 | 1.00 | 20.91 | O |
| ATOM | 3943 | N | THR | I | 77 | 35.229 | −47.571 | −8.278 | 1.00 | 20.47 | N |
| ATOM | 3944 | CA | THR | I | 77 | 36.231 | −46.827 | −9.039 | 1.00 | 19.99 | C |
| ATOM | 3945 | CB | THR | I | 77 | 37.589 | −47.540 | −8.990 | 1.00 | 19.91 | C |
| ATOM | 3946 | OG1 | THR | I | 77 | 37.901 | −47.853 | −7.625 | 1.00 | 20.34 | O |
| ATOM | 3947 | CG2 | THR | I | 77 | 38.680 | −46.666 | −9.560 | 1.00 | 19.77 | C |
| ATOM | 3948 | C | THR | I | 77 | 35.794 | −46.615 | −10.494 | 1.00 | 19.67 | C |
| ATOM | 3949 | O | THR | I | 77 | 35.376 | −47.554 | −11.161 | 1.00 | 19.54 | O |
| ATOM | 3950 | N | ALA | I | 78 | 35.898 | −45.378 | −10.964 | 1.00 | 19.25 | N |
| ATOM | 3951 | CA | ALA | I | 78 | 35.689 | −45.048 | −12.372 | 1.00 | 19.51 | C |
| ATOM | 3952 | CB | ALA | I | 78 | 34.890 | −43.766 | −12.506 | 1.00 | 19.81 | C |
| ATOM | 3953 | C | ALA | I | 78 | 37.063 | −44.874 | −13.011 | 1.00 | 19.93 | C |
| ATOM | 3954 | O | ALA | I | 78 | 38.019 | −44.460 | −12.334 | 1.00 | 19.85 | O |
| ATOM | 3955 | N | TYR | I | 79 | 37.159 | −45.226 | −14.294 | 1.00 | 18.96 | N |
| ATOM | 3956 | CA | TYR | I | 79 | 38.427 | −45.197 | −15.017 | 1.00 | 19.14 | C |
| ATOM | 3957 | CB | TYR | I | 79 | 38.934 | −46.608 | −15.365 | 1.00 | 18.55 | C |
| ATOM | 3958 | CG | TYR | I | 79 | 39.248 | −47.485 | −14.180 | 1.00 | 20.08 | C |
| ATOM | 3959 | CD1 | TYR | I | 79 | 40.484 | −47.411 | −13.530 | 1.00 | 19.65 | C |
| ATOM | 3960 | CE1 | TYR | I | 79 | 40.760 | −48.253 | −12.419 | 1.00 | 23.07 | C |
| ATOM | 3961 | CZ | TYR | I | 79 | 39.793 | −49.141 | −11.979 | 1.00 | 21.72 | C |
| ATOM | 3962 | OH | TYR | I | 79 | 40.017 | −49.970 | −10.901 | 1.00 | 24.79 | O |
| ATOM | 3963 | CE2 | TYR | I | 79 | 38.575 | −49.224 | −12.619 | 1.00 | 22.26 | C |
| ATOM | 3964 | CD2 | TYR | I | 79 | 38.309 | −48.405 | −13.714 | 1.00 | 19.86 | C |
| ATOM | 3965 | C | TYR | I | 79 | 38.290 | −44.424 | −16.313 | 1.00 | 18.95 | C |
| ATOM | 3966 | O | TYR | I | 79 | 37.241 | −44.456 | −16.970 | 1.00 | 18.97 | O |
| ATOM | 3967 | N | LEU | I | 80 | 39.376 | −43.754 | −16.677 | 1.00 | 19.18 | N |
| ATOM | 3968 | CA | LEU | I | 80 | 39.530 | −43.159 | −18.003 | 1.00 | 19.48 | C |
| ATOM | 3969 | CB | LEU | I | 80 | 39.732 | −41.627 | −17.900 | 1.00 | 18.96 | C |
| ATOM | 3970 | CG | LEU | I | 80 | 40.070 | −40.888 | −19.208 | 1.00 | 19.71 | C |
| ATOM | 3971 | CD1 | LEU | I | 80 | 38.975 | −41.052 | −20.256 | 1.00 | 21.34 | C |
| ATOM | 3972 | CD2 | LEU | I | 80 | 40.356 | −39.417 | −18.945 | 1.00 | 18.80 | C |
| ATOM | 3973 | C | LEU | I | 80 | 40.717 | −43.824 | −18.697 | 1.00 | 19.99 | C |
| ATOM | 3974 | O | LEU | I | 80 | 41.860 | −43.741 | −18.234 | 1.00 | 19.82 | O |
| ATOM | 3975 | N | GLN | I | 81 | 40.420 | −44.514 | −19.792 | 1.00 | 20.90 | N |
| ATOM | 3976 | CA | GLN | I | 81 | 41.434 | −45.110 | −20.646 | 1.00 | 22.12 | C |
| ATOM | 3977 | CB | GLN | I | 81 | 40.973 | −46.466 | −21.163 | 1.00 | 22.64 | C |
| ATOM | 3978 | CG | GLN | I | 81 | 41.271 | −47.626 | −20.273 | 1.00 | 26.33 | C |
| ATOM | 3979 | CD | GLN | I | 81 | 40.725 | −48.908 | −20.850 | 1.00 | 30.18 | C |
| ATOM | 3980 | OE1 | GLN | I | 81 | 41.453 | −49.660 | −21.513 | 1.00 | 32.19 | O |
| ATOM | 3981 | NE2 | GLN | I | 81 | 39.424 | −49.146 | −20.649 | 1.00 | 29.51 | N |
| ATOM | 3982 | C | GLN | I | 81 | 41.618 | −44.214 | −21.845 | 1.00 | 22.01 | C |
| ATOM | 3983 | O | GLN | I | 81 | 40.630 | −43.740 | −22.427 | 1.00 | 21.78 | O |
| ATOM | 3984 | N | TRP | I | 82 | 42.879 | −44.007 | −22.226 | 1.00 | 21.21 | N |
| ATOM | 3985 | CA | TRP | I | 82 | 43.207 | −43.161 | −23.369 | 1.00 | 20.40 | C |
| ATOM | 3986 | CB | TRP | I | 82 | 43.622 | −41.751 | −22.900 | 1.00 | 20.05 | C |
| ATOM | 3987 | CG | TRP | I | 82 | 43.403 | −40.667 | −23.924 | 1.00 | 19.33 | C |
| ATOM | 3988 | CD1 | TRP | I | 82 | 44.031 | −40.537 | −25.128 | 1.00 | 21.29 | C |
| ATOM | 3989 | NE1 | TRP | I | 82 | 43.567 | −39.425 | −25.792 | 1.00 | 20.86 | N |
| ATOM | 3990 | CE2 | TRP | I | 82 | 42.621 | −38.809 | −25.014 | 1.00 | 19.31 | C |
| ATOM | 3991 | CD2 | TRP | I | 82 | 42.493 | −39.564 | −23.825 | 1.00 | 19.68 | C |
| ATOM | 3992 | CE3 | TRP | I | 82 | 41.564 | −39.151 | −22.852 | 1.00 | 19.92 | C |
| ATOM | 3993 | CZ3 | TRP | I | 82 | 40.810 | −37.998 | −23.094 | 1.00 | 20.26 | C |
| ATOM | 3994 | CH2 | TRP | I | 82 | 40.968 | −37.266 | −24.298 | 1.00 | 19.79 | C |
| ATOM | 3995 | CZ2 | TRP | I | 82 | 41.863 | −37.659 | −25.262 | 1.00 | 19.72 | C |
| ATOM | 3996 | C | TRP | I | 82 | 44.320 | −43.829 | −24.163 | 1.00 | 20.75 | C |
| ATOM | 3997 | O | TRP | I | 82 | 45.427 | −44.009 | −23.652 | 1.00 | 20.38 | O |
| ATOM | 3998 | N | SER | I | 82A | 44.004 | −44.222 | −25.397 | 1.00 | 20.27 | N |
| ATOM | 3999 | CA | SER | I | 82A | 44.979 | −44.815 | −26.312 | 1.00 | 20.01 | C |
| ATOM | 4000 | CB | SER | I | 82A | 44.340 | −45.978 | −27.069 | 1.00 | 20.29 | C |
| ATOM | 4001 | OG | SER | I | 82A | 43.223 | −45.521 | −27.810 | 1.00 | 21.08 | O |
| ATOM | 4002 | C | SER | I | 82A | 45.541 | −43.782 | −27.305 | 1.00 | 19.80 | C |
| ATOM | 4003 | O | SER | I | 82A | 45.022 | −42.665 | −27.419 | 1.00 | 19.19 | O |
| ATOM | 4004 | N | SER | I | 82B | 46.601 | −44.177 | −28.015 | 1.00 | 19.24 | N |
| ATOM | 4005 | CA | SER | I | 82B | 47.279 | −43.330 | −29.003 | 1.00 | 19.30 | C |
| ATOM | 4006 | CB | SER | I | 82B | 46.466 | −43.234 | −30.306 | 1.00 | 19.48 | C |
| ATOM | 4007 | OG | SER | I | 82B | 46.273 | −44.504 | −30.853 | 1.00 | 20.94 | O |
| ATOM | 4008 | C | SER | I | 82B | 47.547 | −41.939 | −28.498 | 1.00 | 18.66 | C |
| ATOM | 4009 | O | SER | I | 82B | 47.181 | −40.962 | −29.164 | 1.00 | 19.08 | O |
| ATOM | 4010 | N | LEU | I | 82C | 48.164 | −41.845 | −27.318 | 1.00 | 17.61 | N |
| ATOM | 4011 | CA | LEU | I | 82C | 48.488 | −40.561 | −26.723 | 1.00 | 17.10 | C |
| ATOM | 4012 | CB | LEU | I | 82C | 49.146 | −40.760 | −25.364 | 1.00 | 17.30 | C |
| ATOM | 4013 | CG | LEU | I | 82C | 48.253 | −41.275 | −24.248 | 1.00 | 15.62 | C |
| ATOM | 4014 | CD1 | LEU | I | 82C | 49.143 | −41.948 | −23.220 | 1.00 | 16.39 | C |
| ATOM | 4015 | CD2 | LEU | I | 82C | 47.432 | −40.145 | −23.645 | 1.00 | 16.45 | C |
| ATOM | 4016 | C | LEU | I | 82C | 49.404 | −39.714 | −27.614 | 1.00 | 17.50 | C |
| ATOM | 4017 | O | LEU | I | 82C | 50.254 | −40.248 | −28.318 | 1.00 | 16.75 | O |
| ATOM | 4018 | N | LYS | I | 83 | 49.208 | −38.394 | −27.557 | 1.00 | 17.88 | N |
| ATOM | 4019 | CA | LYS | I | 83 | 49.923 | −37.415 | −28.396 | 1.00 | 19.05 | C |
| ATOM | 4020 | CB | LYS | I | 83 | 48.950 | −36.729 | −29.360 | 1.00 | 19.22 | C |
| ATOM | 4021 | CG | LYS | I | 83 | 48.377 | −37.633 | −30.420 | 1.00 | 23.21 | C |
| ATOM | 4022 | CD | LYS | I | 83 | 46.902 | −37.333 | −30.650 | 1.00 | 28.29 | C |
| ATOM | 4023 | CE | LYS | I | 83 | 46.691 | −36.069 | −31.475 | 1.00 | 29.48 | C |
| ATOM | 4024 | NZ | LYS | I | 83 | 45.245 | −35.748 | −31.558 | 1.00 | 29.84 | N |
| ATOM | 4025 | C | LYS | I | 83 | 50.414 | −36.359 | −27.439 | 1.00 | 18.50 | C |
| ATOM | 4026 | O | LYS | I | 83 | 49.863 | −36.243 | −26.352 | 1.00 | 17.62 | O |
| ATOM | 4027 | N | ALA | I | 84 | 51.417 | −35.583 | −27.841 | 1.00 | 18.31 | N |
| ATOM | 4028 | CA | ALA | I | 84 | 51.889 | −34.439 | −27.051 | 1.00 | 18.67 | C |
| ATOM | 4029 | CB | ALA | I | 84 | 52.911 | −33.614 | −27.864 | 1.00 | 18.71 | C |
| ATOM | 4030 | C | ALA | I | 84 | 50.742 | −33.535 | −26.560 | 1.00 | 19.03 | C |
| ATOM | 4031 | O | ALA | I | 84 | 50.731 | −33.081 | −25.409 | 1.00 | 18.03 | O |
| ATOM | 4032 | N | SER | I | 85 | 49.766 | −33.309 | −27.435 | 1.00 | 19.40 | N |
| ATOM | 4033 | CA | SER | I | 85 | 48.682 | −32.366 | −27.159 | 1.00 | 20.34 | C |
| ATOM | 4034 | CB | SER | I | 85 | 47.950 | −32.049 | −28.468 | 1.00 | 20.02 | C |
| ATOM | 4035 | OG | SER | I | 85 | 47.366 | −33.236 | −28.958 | 1.00 | 19.81 | O |
| ATOM | 4036 | C | SER | I | 85 | 47.692 | −32.886 | −26.090 | 1.00 | 20.97 | C |
| ATOM | 4037 | O | SER | I | 85 | 46.793 | −32.143 | −25.638 | 1.00 | 20.93 | O |
| ATOM | 4038 | N | ASP | I | 86 | 47.852 | −34.151 | −25.687 | 1.00 | 20.56 | N |
| ATOM | 4039 | CA | ASP | I | 86 | 47.045 | −34.726 | −24.598 | 1.00 | 20.58 | C |
| ATOM | 4040 | CB | ASP | I | 86 | 46.928 | −36.257 | −24.744 | 1.00 | 20.74 | C |
| ATOM | 4041 | CG | ASP | I | 86 | 46.119 | −36.668 | −25.956 | 1.00 | 20.65 | C |
| ATOM | 4042 | OD1 | ASP | I | 86 | 45.101 | −36.002 | −26.249 | 1.00 | 20.55 | O |
| ATOM | 4043 | OD2 | ASP | I | 86 | 46.499 | −37.667 | −26.613 | 1.00 | 22.12 | O |
| ATOM | 4044 | C | ASP | I | 86 | 47.612 | −34.369 | −23.217 | 1.00 | 20.72 | C |
| ATOM | 4045 | O | ASP | I | 86 | 47.002 | −34.690 | −22.189 | 1.00 | 20.58 | O |
| ATOM | 4046 | N | THR | I | 87 | 48.770 | −33.703 | −23.199 | 1.00 | 20.53 | N |
| ATOM | 4047 | CA | THR | I | 87 | 49.359 | −33.193 | −21.955 | 1.00 | 20.69 | C |
| ATOM | 4048 | CB | THR | I | 87 | 50.705 | −32.476 | −22.221 | 1.00 | 20.85 | C |
| ATOM | 4049 | OG1 | THR | I | 87 | 51.644 | −33.422 | −22.747 | 1.00 | 22.06 | O |
| ATOM | 4050 | CG2 | THR | I | 87 | 51.278 | −31.827 | −20.944 | 1.00 | 20.04 | C |
| ATOM | 4051 | C | THR | I | 87 | 48.362 | −32.242 | −21.295 | 1.00 | 20.76 | C |
| ATOM | 4052 | O | THR | I | 87 | 47.912 | −31.280 | −21.910 | 1.00 | 20.73 | O |
| ATOM | 4053 | N | ALA | I | 88 | 47.995 | −32.544 | −20.055 | 1.00 | 20.81 | N |
| ATOM | 4054 | CA | ALA | I | 88 | 46.987 | −31.759 | −19.334 | 1.00 | 21.09 | C |
| ATOM | 4055 | CB | ALA | I | 88 | 45.688 | −31.688 | −20.132 | 1.00 | 20.88 | C |
| ATOM | 4056 | C | ALA | I | 88 | 46.719 | −32.327 | −17.951 | 1.00 | 20.84 | C |
| ATOM | 4057 | O | ALA | I | 88 | 47.180 | −33.432 | −17.612 | 1.00 | 20.69 | O |
| ATOM | 4058 | N | MET | I | 89 | 45.983 | −31.557 | −17.144 | 1.00 | 20.67 | N |
| ATOM | 4059 | CA | MET | I | 89 | 45.438 | −32.064 | −15.899 | 1.00 | 20.16 | C |
| ATOM | 4060 | CB | MET | I | 89 | 45.277 | −30.949 | −14.867 | 1.00 | 20.52 | C |
| ATOM | 4061 | CG | MET | I | 89 | 44.756 | −31.461 | −13.527 | 1.00 | 21.06 | C |
| ATOM | 4062 | SD | MET | I | 89 | 46.053 | −32.290 | −12.601 | 1.00 | 25.50 | S |
| ATOM | 4063 | CE | MET | I | 89 | 47.067 | −30.883 | −12.095 | 1.00 | 28.60 | C |
| ATOM | 4064 | C | MET | I | 89 | 44.069 | −32.671 | −16.188 | 1.00 | 20.18 | C |
| ATOM | 4065 | O | MET | I | 89 | 43.209 | −32.008 | −16.771 | 1.00 | 20.30 | O |
| ATOM | 4066 | N | TYR | I | 90 | 43.877 | −33.921 | −15.767 | 1.00 | 19.78 | N |
| ATOM | 4067 | CA | TYR | I | 90 | 42.613 | −34.631 | −15.949 | 1.00 | 19.85 | C |
| ATOM | 4068 | CB | TYR | I | 90 | 42.886 | −36.048 | −16.464 | 1.00 | 19.22 | C |
| ATOM | 4069 | CG | TYR | I | 90 | 43.378 | −36.029 | −17.896 | 1.00 | 19.99 | C |
| ATOM | 4070 | CD1 | TYR | I | 90 | 44.640 | −35.516 | −18.215 | 1.00 | 18.63 | C |
| ATOM | 4071 | CE1 | TYR | I | 90 | 45.094 | −35.474 | −19.543 | 1.00 | 19.33 | C |
| ATOM | 4072 | CZ | TYR | I | 90 | 44.272 | −35.933 | −20.555 | 1.00 | 17.95 | C |
| ATOM | 4073 | OH | TYR | I | 90 | 44.699 | −35.894 | −21.863 | 1.00 | 19.99 | O |
| ATOM | 4074 | CE2 | TYR | I | 90 | 43.016 | −36.442 | −20.269 | 1.00 | 19.80 | C |
| ATOM | 4075 | CD2 | TYR | I | 90 | 42.568 | −36.483 | −18.935 | 1.00 | 19.40 | C |
| ATOM | 4076 | C | TYR | I | 90 | 41.863 | −34.717 | −14.624 | 1.00 | 19.58 | C |
| ATOM | 4077 | O | TYR | I | 90 | 42.388 | −35.290 | −13.664 | 1.00 | 21.07 | O |
| ATOM | 4078 | N | PHE | I | 91 | 40.657 | −34.162 | −14.592 | 1.00 | 19.28 | N |
| ATOM | 4079 | CA | PHE | I | 91 | 39.770 | −34.178 | −13.422 | 1.00 | 19.63 | C |
| ATOM | 4080 | CB | PHE | I | 91 | 39.121 | −32.804 | −13.221 | 1.00 | 19.51 | C |
| ATOM | 4081 | CG | PHE | I | 91 | 40.070 | −31.718 | −12.783 | 1.00 | 20.47 | C |
| ATOM | 4082 | CD1 | PHE | I | 91 | 40.565 | −31.683 | −11.473 | 1.00 | 20.27 | C |
| ATOM | 4083 | CE1 | PHE | I | 91 | 41.428 | −30.675 | −11.065 | 1.00 | 20.39 | C |
| ATOM | 4084 | CZ | PHE | I | 91 | 41.776 | −29.661 | −11.959 | 1.00 | 21.46 | C |
| ATOM | 4085 | CE2 | PHE | I | 91 | 41.265 | −29.672 | −13.263 | 1.00 | 19.91 | C |
| ATOM | 4086 | CD2 | PHE | I | 91 | 40.412 | −30.700 | −13.659 | 1.00 | 21.01 | C |
| ATOM | 4087 | C | PHE | I | 91 | 38.614 | −35.160 | −13.633 | 1.00 | 19.94 | C |
| ATOM | 4088 | O | PHE | I | 91 | 38.083 | −35.258 | −14.739 | 1.00 | 19.41 | O |
| ATOM | 4089 | N | CYS | I | 92 | 38.207 | −35.853 | −12.575 | 1.00 | 20.05 | N |
| ATOM | 4090 | CA | CYS | I | 92 | 36.866 | −36.456 | −12.557 | 1.00 | 20.61 | C |
| ATOM | 4091 | CB | CYS | I | 92 | 36.887 | −37.904 | −12.082 | 1.00 | 21.05 | C |
| ATOM | 4092 | SG | CYS | I | 92 | 37.498 | −38.141 | −10.404 | 1.00 | 23.89 | S |
| ATOM | 4093 | C | CYS | I | 92 | 35.957 | −35.589 | −11.682 | 1.00 | 20.19 | C |
| ATOM | 4094 | O | CYS | I | 92 | 36.436 | −34.868 | −10.775 | 1.00 | 18.99 | O |
| ATOM | 4095 | N | ALA | I | 93 | 34.659 | −35.610 | −11.970 | 1.00 | 19.77 | N |
| ATOM | 4096 | CA | ALA | I | 93 | 33.709 | −34.855 | −11.154 | 1.00 | 19.01 | C |
| ATOM | 4097 | CB | ALA | I | 93 | 33.570 | −33.447 | −11.665 | 1.00 | 18.69 | C |
| ATOM | 4098 | C | ALA | I | 93 | 32.346 | −35.515 | −11.093 | 1.00 | 19.22 | C |
| ATOM | 4099 | O | ALA | I | 93 | 31.946 | −36.225 | −12.020 | 1.00 | 19.55 | O |
| ATOM | 4100 | N | ARG | I | 94 | 31.651 | −35.289 | −9.988 | 1.00 | 18.46 | N |
| ATOM | 4101 | CA | ARG | I | 94 | 30.233 | −35.622 | −9.914 | 1.00 | 19.01 | C |
| ATOM | 4102 | CB | ARG | I | 94 | 29.921 | −36.715 | −8.900 | 1.00 | 18.44 | C |
| ATOM | 4103 | CG | ARG | I | 94 | 28.419 | −36.977 | −8.856 | 1.00 | 22.12 | C |
| ATOM | 4104 | CD | ARG | I | 94 | 28.118 | −38.151 | −8.042 | 1.00 | 28.19 | C |
| ATOM | 4105 | NE | ARG | I | 94 | 28.089 | −37.874 | −6.632 | 1.00 | 29.09 | N |
| ATOM | 4106 | CZ | ARG | I | 94 | 27.001 | −38.017 | −5.881 | 1.00 | 33.98 | C |
| ATOM | 4107 | NH1 | ARG | I | 94 | 27.058 | −37.749 | −4.597 | 1.00 | 30.08 | N |
| ATOM | 4108 | NH2 | ARG | I | 94 | 25.850 | −38.431 | −6.420 | 1.00 | 37.66 | N |
| ATOM | 4109 | C | ARG | I | 94 | 29.449 | −34.381 | −9.570 | 1.00 | 18.17 | C |
| ATOM | 4110 | O | ARG | I | 94 | 29.642 | −33.770 | −8.508 | 1.00 | 18.35 | O |
| ATOM | 4111 | N | ARG | I | 95 | 28.544 | −34.038 | −10.477 | 1.00 | 18.12 | N |
| ATOM | 4112 | CA | ARG | I | 95 | 27.771 | −32.804 | −10.400 | 1.00 | 17.70 | C |
| ATOM | 4113 | CB | ARG | I | 95 | 28.140 | −31.896 | −11.583 | 1.00 | 17.51 | C |
| ATOM | 4114 | CG | ARG | I | 95 | 29.642 | −31.694 | −11.763 | 1.00 | 16.98 | C |
| ATOM | 4115 | CD | ARG | I | 95 | 29.953 | −30.509 | −12.681 | 1.00 | 17.60 | C |
| ATOM | 4116 | NE | ARG | I | 95 | 29.717 | −30.788 | −14.101 | 1.00 | 16.59 | N |
| ATOM | 4117 | CZ | ARG | I | 95 | 29.504 | −29.847 | −15.018 | 1.00 | 17.27 | C |
| ATOM | 4118 | NH1 | ARG | I | 95 | 29.484 | −28.565 | −14.662 | 1.00 | 16.38 | N |
| ATOM | 4119 | NH2 | ARG | I | 95 | 29.320 | −30.180 | −16.299 | 1.00 | 15.89 | N |
| ATOM | 4120 | C | ARG | I | 95 | 26.269 | −33.065 | −10.419 | 1.00 | 17.88 | C |
| ATOM | 4121 | O | ARG | I | 95 | 25.519 | −32.371 | −9.755 | 1.00 | 18.90 | O |
| ATOM | 4122 | N | PHE | I | 96 | 25.833 | −34.065 | −11.178 | 1.00 | 18.52 | N |
| ATOM | 4123 | CA | PHE | I | 96 | 24.408 | −34.197 | −11.506 | 1.00 | 18.82 | C |
| ATOM | 4124 | CB | PHE | I | 96 | 24.180 | −33.883 | −12.993 | 1.00 | 18.68 | C |
| ATOM | 4125 | CG | PHE | I | 96 | 24.737 | −32.578 | −13.435 | 1.00 | 18.57 | C |
| ATOM | 4126 | CD1 | PHE | I | 96 | 24.321 | −31.383 | −12.844 | 1.00 | 16.91 | C |
| ATOM | 4127 | CE1 | PHE | I | 96 | 24.849 | −30.167 | −13.258 | 1.00 | 18.54 | C |
| ATOM | 4128 | CZ | PHE | I | 96 | 25.782 | −30.132 | −14.291 | 1.00 | 16.49 | C |
| ATOM | 4129 | CE2 | PHE | I | 96 | 26.189 | −31.294 | −14.885 | 1.00 | 17.54 | C |
| ATOM | 4130 | CD2 | PHE | I | 96 | 25.660 | −32.528 | −14.466 | 1.00 | 18.69 | C |
| ATOM | 4131 | C | PHE | I | 96 | 23.773 | −35.554 | −11.225 | 1.00 | 18.52 | C |
| ATOM | 4132 | O | PHE | I | 96 | 24.448 | −36.582 | −11.166 | 1.00 | 18.47 | O |
| ATOM | 4133 | N | GLY | I | 97 | 22.452 | −35.530 | −11.063 | 1.00 | 19.59 | N |
| ATOM | 4134 | CA | GLY | I | 97 | 21.628 | −36.733 | −11.101 | 1.00 | 19.83 | C |
| ATOM | 4135 | C | GLY | I | 97 | 20.907 | −36.747 | −12.435 | 1.00 | 20.36 | C |
| ATOM | 4136 | O | GLY | I | 97 | 21.399 | −36.171 | −13.415 | 1.00 | 21.34 | O |
| ATOM | 4137 | N | TYR | I | 98 | 19.748 | −37.390 | −12.503 | 1.00 | 19.22 | N |
| ATOM | 4138 | CA | TYR | I | 98 | 18.941 | −37.276 | −13.714 | 1.00 | 18.99 | C |
| ATOM | 4139 | CB | TYR | I | 98 | 18.054 | −38.523 | −13.922 | 1.00 | 18.43 | C |
| ATOM | 4140 | CG | TYR | I | 98 | 17.418 | −38.564 | −15.294 | 1.00 | 20.29 | C |
| ATOM | 4141 | CD1 | TYR | I | 98 | 18.161 | −38.928 | −16.423 | 1.00 | 21.41 | C |
| ATOM | 4142 | CE1 | TYR | I | 98 | 17.576 | −38.962 | −17.695 | 1.00 | 22.31 | C |
| ATOM | 4143 | CZ | TYR | I | 98 | 16.244 | −38.619 | −17.832 | 1.00 | 23.50 | C |
| ATOM | 4144 | OH | TYR | I | 98 | 15.634 | −38.652 | −19.063 | 1.00 | 24.82 | O |
| ATOM | 4145 | CE2 | TYR | I | 98 | 15.489 | −38.262 | −16.723 | 1.00 | 22.74 | C |
| ATOM | 4146 | CD2 | TYR | I | 98 | 16.083 | −38.229 | −15.471 | 1.00 | 21.61 | C |
| ATOM | 4147 | C | TYR | I | 98 | 18.106 | −35.986 | −13.648 | 1.00 | 18.59 | C |
| ATOM | 4148 | O | TYR | I | 98 | 17.800 | −35.488 | −12.550 | 1.00 | 17.89 | O |
| ATOM | 4149 | N | TYR | I | 99 | 17.755 | −35.431 | −14.810 | 1.00 | 18.05 | N |
| ATOM | 4150 | CA | TYR | I | 99 | 16.860 | −34.273 | −14.860 | 1.00 | 17.81 | C |
| ATOM | 4151 | CB | TYR | I | 99 | 16.418 | −33.958 | −16.305 | 1.00 | 18.26 | C |
| ATOM | 4152 | CG | TYR | I | 99 | 17.571 | −33.609 | −17.254 | 1.00 | 19.06 | C |
| ATOM | 4153 | CD1 | TYR | I | 99 | 18.243 | −32.379 | −17.149 | 1.00 | 19.75 | C |
| ATOM | 4154 | CE1 | TYR | I | 99 | 19.318 | −32.042 | −18.018 | 1.00 | 20.04 | C |
| ATOM | 4155 | CZ | TYR | I | 99 | 19.715 | −32.948 | −19.005 | 1.00 | 20.17 | C |
| ATOM | 4156 | OH | TYR | I | 99 | 20.774 | −32.631 | −19.836 | 1.00 | 17.81 | O |
| ATOM | 4157 | CE2 | TYR | I | 99 | 19.068 | −34.185 | −19.122 | 1.00 | 18.68 | C |
| ATOM | 4158 | CD2 | TYR | I | 99 | 17.989 | −34.507 | −18.250 | 1.00 | 19.42 | C |
| ATOM | 4159 | C | TYR | I | 99 | 15.643 | −34.520 | −13.972 | 1.00 | 17.97 | C |
| ATOM | 4160 | O | TYR | I | 99 | 15.115 | −35.638 | −13.924 | 1.00 | 17.73 | O |
| ATOM | 4161 | N | GLY | I | 100 | 15.218 | −33.484 | −13.255 | 1.00 | 17.38 | N |
| ATOM | 4162 | CA | GLY | I | 100 | 14.051 | −33.553 | −12.380 | 1.00 | 17.48 | C |
| ATOM | 4163 | C | GLY | I | 100 | 14.337 | −34.044 | −10.973 | 1.00 | 17.15 | C |
| ATOM | 4164 | O | GLY | I | 100 | 13.452 | −34.027 | −10.121 | 1.00 | 17.01 | O |
| ATOM | 4165 | N | SER | I | 100A | 15.566 | −34.495 | −10.735 | 1.00 | 17.24 | N |
| ATOM | 4166 | CA | SER | I | 100A | 15.971 | −35.022 | −9.428 | 1.00 | 17.74 | C |
| ATOM | 4167 | CB | SER | I | 100A | 17.198 | −35.930 | −9.585 | 1.00 | 17.52 | C |
| ATOM | 4168 | OG | SER | I | 100A | 18.359 | −35.173 | −9.981 | 1.00 | 16.68 | O |
| ATOM | 4169 | C | SER | I | 100A | 16.287 | −33.909 | −8.421 | 1.00 | 18.41 | C |
| ATOM | 4170 | O | SER | I | 100A | 16.316 | −34.145 | −7.198 | 1.00 | 18.91 | O |
| ATOM | 4171 | N | GLY | I | 100B | 16.553 | −32.709 | −8.932 | 1.00 | 18.47 | N |
| ATOM | 4172 | CA | GLY | I | 100B | 16.941 | −31.582 | −8.086 | 1.00 | 18.73 | C |
| ATOM | 4173 | C | GLY | I | 100B | 18.349 | −31.723 | −7.522 | 1.00 | 19.09 | C |
| ATOM | 4174 | O | GLY | I | 100B | 18.760 | −30.940 | −6.674 | 1.00 | 19.05 | O |
| ATOM | 4175 | N | ASN | I | 100C | 19.076 | −32.738 | −7.978 | 1.00 | 18.76 | N |
| ATOM | 4176 | CA | ASN | I | 100C | 20.445 | −32.980 | −7.539 | 1.00 | 19.42 | C |
| ATOM | 4177 | CB | ASN | I | 100C | 20.868 | −34.409 | −7.891 | 1.00 | 19.15 | C |
| ATOM | 4178 | CG | ASN | I | 100C | 20.218 | −35.438 | −7.014 | 1.00 | 22.08 | C |
| ATOM | 4179 | OD1 | ASN | I | 100C | 19.952 | −35.194 | −5.846 | 1.00 | 22.96 | O |
| ATOM | 4180 | ND2 | ASN | I | 100C | 19.958 | −36.607 | −7.576 | 1.00 | 24.06 | N |
| ATOM | 4181 | C | ASN | I | 100C | 21.467 | −32.045 | −8.164 | 1.00 | 18.35 | C |
| ATOM | 4182 | O | ASN | I | 100C | 21.643 | −32.052 | −9.381 | 1.00 | 17.61 | O |
| ATOM | 4183 | N | TYR | I | 100D | 22.146 | −31.268 | −7.321 | 1.00 | 18.43 | N |
| ATOM | 4184 | CA | TYR | I | 100D | 23.339 | −30.516 | −7.727 | 1.00 | 17.64 | C |
| ATOM | 4185 | CB | TYR | I | 100D | 23.016 | −29.008 | −7.868 | 1.00 | 18.12 | C |
| ATOM | 4186 | CG | TYR | I | 100D | 22.126 | −28.817 | −9.056 | 1.00 | 17.16 | C |
| ATOM | 4187 | CD1 | TYR | I | 100D | 22.677 | −28.696 | −10.341 | 1.00 | 16.26 | C |
| ATOM | 4188 | CE1 | TYR | I | 100D | 21.868 | −28.594 | −11.450 | 1.00 | 15.84 | C |
| ATOM | 4189 | CZ | TYR | I | 100D | 20.489 | −28.628 | −11.288 | 1.00 | 15.63 | C |
| ATOM | 4190 | OH | TYR | I | 100D | 19.684 | −28.541 | −12.384 | 1.00 | 17.03 | O |
| ATOM | 4191 | CE2 | TYR | I | 100D | 19.911 | −28.765 | −10.026 | 1.00 | 15.92 | C |
| ATOM | 4192 | CD2 | TYR | I | 100D | 20.733 | −28.878 | −8.923 | 1.00 | 15.16 | C |
| ATOM | 4193 | C | TYR | I | 100D | 24.469 | −30.784 | −6.741 | 1.00 | 17.64 | C |
| ATOM | 4194 | O | TYR | I | 100D | 24.341 | −30.474 | −5.555 | 1.00 | 16.78 | O |
| ATOM | 4195 | N | PHE | I | 100E | 25.544 | −31.399 | −7.238 | 1.00 | 17.01 | N |
| ATOM | 4196 | CA | PHE | I | 100E | 26.719 | −31.711 | −6.426 | 1.00 | 17.69 | C |
| ATOM | 4197 | CB | PHE | I | 100E | 27.010 | −33.218 | −6.445 | 1.00 | 17.11 | C |
| ATOM | 4198 | CG | PHE | I | 100E | 25.802 | −34.077 | −6.140 | 1.00 | 19.21 | C |
| ATOM | 4199 | CD1 | PHE | I | 100E | 25.343 | −34.219 | −4.818 | 1.00 | 19.45 | C |
| ATOM | 4200 | CE1 | PHE | I | 100E | 24.222 | −35.011 | −4.532 | 1.00 | 19.56 | C |
| ATOM | 4201 | CZ | PHE | I | 100E | 23.558 | −35.669 | −5.565 | 1.00 | 20.05 | C |
| ATOM | 4202 | CE2 | PHE | I | 100E | 24.005 | −35.540 | −6.883 | 1.00 | 20.58 | C |
| ATOM | 4203 | CD2 | PHE | I | 100E | 25.126 | −34.744 | −7.165 | 1.00 | 18.95 | C |
| ATOM | 4204 | C | PHE | I | 100E | 27.941 | −30.944 | −6.936 | 1.00 | 17.58 | C |
| ATOM | 4205 | O | PHE | I | 100E | 27.934 | −30.401 | −8.044 | 1.00 | 17.70 | O |
| ATOM | 4206 | N | ASP | I | 101 | 28.990 | −30.899 | −6.130 | 1.00 | 17.55 | N |
| ATOM | 4207 | CA | ASP | I | 101 | 30.231 | −30.288 | −6.582 | 1.00 | 16.98 | C |
| ATOM | 4208 | CB | ASP | I | 101 | 30.298 | −28.836 | −6.109 | 1.00 | 17.36 | C |
| ATOM | 4209 | CG | ASP | I | 101 | 31.328 | −28.015 | −6.856 | 1.00 | 16.49 | C |
| ATOM | 4210 | OD1 | ASP | I | 101 | 31.467 | −28.179 | −8.083 | 1.00 | 16.93 | O |
| ATOM | 4211 | OD2 | ASP | I | 101 | 31.988 | −27.182 | −6.207 | 1.00 | 15.35 | O |
| ATOM | 4212 | C | ASP | I | 101 | 31.423 | −31.070 | −6.078 | 1.00 | 17.66 | C |
| ATOM | 4213 | O | ASP | I | 101 | 32.295 | −30.528 | −5.390 | 1.00 | 17.51 | O |
| ATOM | 4214 | N | TYR | I | 102 | 31.467 | −32.353 | −6.424 | 1.00 | 17.13 | N |
| ATOM | 4215 | CA | TYR | I | 102 | 32.585 | −33.182 | −6.008 | 1.00 | 17.58 | C |
| ATOM | 4216 | CB | TYR | I | 102 | 32.100 | −34.573 | −5.584 | 1.00 | 17.35 | C |
| ATOM | 4217 | CG | TYR | I | 102 | 31.103 | −34.508 | −4.423 | 1.00 | 19.28 | C |
| ATOM | 4218 | CD1 | TYR | I | 102 | 31.507 | −34.078 | −3.153 | 1.00 | 20.85 | C |
| ATOM | 4219 | CE1 | TYR | I | 102 | 30.602 | −34.014 | −2.085 | 1.00 | 22.03 | C |
| ATOM | 4220 | CZ | TYR | I | 102 | 29.285 | −34.390 | −2.286 | 1.00 | 20.76 | C |
| ATOM | 4221 | OH | TYR | I | 102 | 28.403 | −34.323 | −1.235 | 1.00 | 24.53 | O |
| ATOM | 4222 | CE2 | TYR | I | 102 | 28.855 | −34.806 | −3.527 | 1.00 | 20.27 | C |
| ATOM | 4223 | CD2 | TYR | I | 102 | 29.771 | −34.871 | −4.599 | 1.00 | 19.51 | C |
| ATOM | 4224 | C | TYR | I | 102 | 33.589 | −33.262 | −7.136 | 1.00 | 17.44 | C |
| ATOM | 4225 | O | TYR | I | 102 | 33.218 | −33.572 | −8.261 | 1.00 | 18.56 | O |
| ATOM | 4226 | N | TRP | I | 103 | 34.848 | −32.968 | −6.836 | 1.00 | 16.68 | N |
| ATOM | 4227 | CA | TRP | I | 103 | 35.928 | −33.028 | −7.840 | 1.00 | 16.90 | C |
| ATOM | 4228 | CB | TRP | I | 103 | 36.464 | −31.620 | −8.141 | 1.00 | 16.99 | C |
| ATOM | 4229 | CG | TRP | I | 103 | 35.483 | −30.787 | −8.869 | 1.00 | 16.63 | C |
| ATOM | 4230 | CD1 | TRP | I | 103 | 34.380 | −30.175 | −8.343 | 1.00 | 16.04 | C |
| ATOM | 4231 | NE1 | TRP | I | 103 | 33.707 | −29.501 | −9.323 | 1.00 | 15.91 | N |
| ATOM | 4232 | CE2 | TRP | I | 103 | 34.365 | −29.663 | −10.512 | 1.00 | 15.66 | C |
| ATOM | 4233 | CD2 | TRP | I | 103 | 35.485 | −30.479 | −10.267 | 1.00 | 16.66 | C |
| ATOM | 4234 | CE3 | TRP | I | 103 | 36.330 | −30.802 | −11.332 | 1.00 | 15.85 | C |
| ATOM | 4235 | CZ3 | TRP | I | 103 | 36.038 | −30.300 | −12.589 | 1.00 | 16.01 | C |
| ATOM | 4236 | CH2 | TRP | I | 103 | 34.905 | −29.494 | −12.805 | 1.00 | 14.38 | C |
| ATOM | 4237 | CZ2 | TRP | I | 103 | 34.067 | −29.156 | −11.783 | 1.00 | 15.97 | C |
| ATOM | 4238 | C | TRP | I | 103 | 37.066 | −33.857 | −7.306 | 1.00 | 16.90 | C |
| ATOM | 4239 | O | TRP | I | 103 | 37.428 | −33.737 | −6.128 | 1.00 | 15.43 | O |
| ATOM | 4240 | N | GLY | I | 104 | 37.654 | −34.674 | −8.183 | 1.00 | 17.20 | N |
| ATOM | 4241 | CA | GLY | I | 104 | 38.937 | −35.315 | −7.874 | 1.00 | 17.91 | C |
| ATOM | 4242 | C | GLY | I | 104 | 40.004 | −34.227 | −7.738 | 1.00 | 18.21 | C |
| ATOM | 4243 | O | GLY | I | 104 | 39.767 | −33.084 | −8.106 | 1.00 | 17.67 | O |
| ATOM | 4244 | N | GLN | I | 105 | 41.178 | −34.597 | −7.231 | 1.00 | 19.73 | N |
| ATOM | 4245 | CA | GLN | I | 105 | 42.300 | −33.660 | −7.071 | 1.00 | 20.48 | C |
| ATOM | 4246 | CB | GLN | I | 105 | 43.285 | −34.181 | −6.010 | 1.00 | 20.94 | C |
| ATOM | 4247 | CG | GLN | I | 105 | 44.289 | −35.227 | −6.524 | 1.00 | 22.86 | C |
| ATOM | 4248 | CD | GLN | I | 105 | 43.806 | −36.665 | −6.437 | 1.00 | 22.87 | C |
| ATOM | 4249 | OE1 | GLN | I | 105 | 42.598 | −36.955 | −6.394 | 1.00 | 22.91 | O |
| ATOM | 4250 | NE2 | GLN | I | 105 | 44.762 | −37.585 | −6.410 | 1.00 | 22.27 | N |
| ATOM | 4251 | C | GLN | I | 105 | 43.004 | −33.403 | −8.412 | 1.00 | 21.40 | C |
| ATOM | 4252 | O | GLN | I | 105 | 43.821 | −32.485 | −8.532 | 1.00 | 21.36 | O |
| ATOM | 4253 | N | GLY | I | 106 | 42.639 | −34.187 | −9.432 | 1.00 | 20.42 | N |
| ATOM | 4254 | CA | GLY | I | 106 | 43.261 | −34.065 | −10.738 | 1.00 | 21.41 | C |
| ATOM | 4255 | C | GLY | I | 106 | 44.461 | −34.987 | −10.863 | 1.00 | 21.29 | C |
| ATOM | 4256 | O | GLY | I | 106 | 45.143 | −35.293 | −9.866 | 1.00 | 21.76 | O |
| ATOM | 4257 | N | THR | I | 107 | 44.685 | −35.464 | −12.083 | 1.00 | 21.64 | N |
| ATOM | 4258 | CA | THR | I | 107 | 45.821 | −36.328 | −12.400 | 1.00 | 21.73 | C |
| ATOM | 4259 | CB | THR | I | 107 | 45.376 | −37.747 | −12.869 | 1.00 | 22.35 | C |
| ATOM | 4260 | OG1 | THR | I | 107 | 44.806 | −38.469 | −11.767 | 1.00 | 21.16 | O |
| ATOM | 4261 | CG2 | THR | I | 107 | 46.579 | −38.550 | −13.428 | 1.00 | 21.32 | C |
| ATOM | 4262 | C | THR | I | 107 | 46.613 | −35.625 | −13.497 | 1.00 | 22.35 | C |
| ATOM | 4263 | O | THR | I | 107 | 46.084 | −35.354 | −14.583 | 1.00 | 21.81 | O |
| ATOM | 4264 | N | MET | I | 108 | 47.878 | −35.315 | −13.218 | 1.00 | 22.30 | N |
| ATOM | 4265 | CA | MET | I | 108 | 48.690 | −34.694 | −14.245 | 1.00 | 23.05 | C |
| ATOM | 4266 | CB | MET | I | 108 | 49.860 | −33.903 | −13.651 | 1.00 | 23.66 | C |
| ATOM | 4267 | CG | MET | I | 108 | 50.546 | −33.030 | −14.692 | 1.00 | 27.73 | C |
| ATOM | 4268 | SD | MET | I | 108 | 49.387 | −31.815 | −15.410 | 1.00 | 36.92 | S |
| ATOM | 4269 | CE | MET | I | 108 | 50.073 | −31.676 | −17.076 | 1.00 | 33.69 | C |
| ATOM | 4270 | C | MET | I | 108 | 49.187 | −35.727 | −15.241 | 1.00 | 21.98 | C |
| ATOM | 4271 | O | MET | I | 108 | 49.736 | −36.764 | −14.860 | 1.00 | 22.38 | O |
| ATOM | 4272 | N | VAL | I | 109 | 48.992 | −35.435 | −16.519 | 1.00 | 20.87 | N |
| ATOM | 4273 | CA | VAL | I | 109 | 49.490 | −36.300 | −17.580 | 1.00 | 20.54 | C |
| ATOM | 4274 | CB | VAL | I | 109 | 48.326 | −36.869 | −18.440 | 1.00 | 20.28 | C |
| ATOM | 4275 | CG1 | VAL | I | 109 | 48.866 | −37.655 | −19.640 | 1.00 | 20.07 | C |
| ATOM | 4276 | CG2 | VAL | I | 109 | 47.400 | −37.764 | −17.596 | 1.00 | 20.65 | C |
| ATOM | 4277 | C | VAL | I | 109 | 50.483 | −35.506 | −18.456 | 1.00 | 20.48 | C |
| ATOM | 4278 | O | VAL | I | 109 | 50.178 | −34.398 | −18.907 | 1.00 | 19.83 | O |
| ATOM | 4279 | N | THR | I | 110 | 51.666 | −36.075 | −18.674 | 1.00 | 20.15 | N |
| ATOM | 4280 | CA | THR | I | 110 | 52.635 | −35.508 | −19.618 | 1.00 | 20.11 | C |
| ATOM | 4281 | CB | THR | I | 110 | 53.960 | −35.127 | −18.912 | 1.00 | 20.52 | C |
| ATOM | 4282 | OG1 | THR | I | 110 | 53.669 | −34.202 | −17.861 | 1.00 | 22.09 | O |
| ATOM | 4283 | CG2 | THR | I | 110 | 54.949 | −34.456 | −19.885 | 1.00 | 21.04 | C |
| ATOM | 4284 | C | THR | I | 110 | 52.889 | −36.548 | −20.700 | 1.00 | 19.83 | C |
| ATOM | 4285 | O | THR | I | 110 | 53.187 | −37.703 | −20.402 | 1.00 | 19.58 | O |
| ATOM | 4286 | N | VAL | I | 111 | 52.723 | −36.140 | −21.952 | 1.00 | 18.83 | N |
| ATOM | 4287 | CA | VAL | I | 111 | 53.047 | −36.984 | −23.080 | 1.00 | 18.70 | C |
| ATOM | 4288 | CB | VAL | I | 111 | 51.838 | −37.212 | −23.998 | 1.00 | 18.68 | C |
| ATOM | 4289 | CG1 | VAL | I | 111 | 52.232 | −38.102 | −25.171 | 1.00 | 18.59 | C |
| ATOM | 4290 | CG2 | VAL | I | 111 | 50.682 | −37.840 | −23.222 | 1.00 | 18.70 | C |
| ATOM | 4291 | C | VAL | I | 111 | 54.167 | −36.311 | −23.876 | 1.00 | 18.65 | C |
| ATOM | 4292 | O | VAL | I | 111 | 53.992 | −35.204 | −24.374 | 1.00 | 17.90 | O |
| ATOM | 4293 | N | SER | I | 112 | 55.304 | −36.994 | −23.996 | 1.00 | 18.95 | N |
| ATOM | 4294 | CA | SER | I | 112 | 56.476 | −36.454 | −24.689 | 1.00 | 19.46 | C |
| ATOM | 4295 | CB | SER | I | 112 | 57.233 | −35.485 | −23.775 | 1.00 | 19.94 | C |
| ATOM | 4296 | OG | SER | I | 112 | 58.477 | −35.117 | −24.366 | 1.00 | 20.62 | O |
| ATOM | 4297 | C | SER | I | 112 | 57.419 | −37.571 | −25.094 | 1.00 | 19.87 | C |
| ATOM | 4298 | O | SER | I | 112 | 57.441 | −38.628 | −24.457 | 1.00 | 19.33 | O |
| ATOM | 4299 | N | SER | I | 113 | 58.203 | −37.323 | −26.150 | 1.00 | 20.26 | N |
| ATOM | 4300 | CA | SER | I | 113 | 59.218 | −38.255 | −26.610 | 1.00 | 20.98 | C |
| ATOM | 4301 | CB | SER | I | 113 | 59.614 | −37.931 | −28.049 | 1.00 | 21.40 | C |
| ATOM | 4302 | OG | SER | I | 113 | 60.091 | −36.596 | −28.117 | 1.00 | 21.11 | O |
| ATOM | 4303 | C | SER | I | 113 | 60.456 | −38.217 | −25.711 | 1.00 | 21.43 | C |
| ATOM | 4304 | O | SER | I | 113 | 61.269 | −39.127 | −25.750 | 1.00 | 21.45 | O |
| ATOM | 4305 | N | ALA | I | 114 | 60.586 | −37.160 | −24.908 | 1.00 | 21.88 | N |
| ATOM | 4306 | CA | ALA | I | 114 | 61.689 | −37.011 | −23.952 | 1.00 | 22.41 | C |
| ATOM | 4307 | CB | ALA | I | 114 | 61.615 | −35.649 | −23.285 | 1.00 | 21.96 | C |
| ATOM | 4308 | C | ALA | I | 114 | 61.702 | −38.102 | −22.884 | 1.00 | 22.69 | C |
| ATOM | 4309 | O | ALA | I | 114 | 60.669 | −38.712 | −22.592 | 1.00 | 22.75 | O |
| ATOM | 4310 | N | SER | I | 115 | 62.872 | −38.315 | −22.284 | 1.00 | 23.14 | N |
| ATOM | 4311 | CA | SER | I | 115 | 63.021 | −39.260 | −21.182 | 1.00 | 23.69 | C |
| ATOM | 4312 | CB | SER | I | 115 | 63.939 | −40.414 | −21.596 | 1.00 | 24.18 | C |
| ATOM | 4313 | OG | SER | I | 115 | 63.332 | −41.189 | −22.621 | 1.00 | 26.53 | O |
| ATOM | 4314 | C | SER | I | 115 | 63.546 | −38.584 | −19.926 | 1.00 | 23.59 | C |
| ATOM | 4315 | O | SER | I | 115 | 64.182 | −37.529 | −19.994 | 1.00 | 23.60 | O |
| ATOM | 4316 | N | THR | I | 116 | 63.276 | −39.200 | −18.780 | 1.00 | 23.84 | N |
| ATOM | 4317 | CA | THR | I | 116 | 63.674 | −38.657 | −17.483 | 1.00 | 24.20 | C |
| ATOM | 4318 | CB | THR | I | 116 | 63.513 | −39.701 | −16.372 | 1.00 | 24.36 | C |
| ATOM | 4319 | OG1 | THR | I | 116 | 62.172 | −40.201 | −16.391 | 1.00 | 25.33 | O |
| ATOM | 4320 | CG2 | THR | I | 116 | 63.792 | −39.090 | −15.008 | 1.00 | 25.17 | C |
| ATOM | 4321 | C | THR | I | 116 | 65.103 | −38.120 | −17.490 | 1.00 | 24.23 | C |
| ATOM | 4322 | O | THR | I | 116 | 66.037 | −38.785 | −17.976 | 1.00 | 23.99 | O |
| ATOM | 4323 | N | LYS | I | 117 | 65.257 | −36.903 | −16.970 | 1.00 | 23.85 | N |
| ATOM | 4324 | CA | LYS | I | 117 | 66.560 | −36.248 | −16.911 | 1.00 | 24.00 | C |
| ATOM | 4325 | CB | LYS | I | 117 | 66.915 | −35.624 | −18.266 | 1.00 | 24.37 | C |
| ATOM | 4326 | CG | LYS | I | 117 | 68.153 | −34.743 | −18.222 | 1.00 | 24.22 | C |
| ATOM | 4327 | CD | LYS | I | 117 | 68.693 | −34.470 | −19.600 | 1.00 | 26.22 | C |
| ATOM | 4328 | CE | LYS | I | 117 | 69.770 | −33.409 | −19.518 | 1.00 | 28.81 | C |
| ATOM | 4329 | NZ | LYS | I | 117 | 70.707 | −33.498 | −20.665 | 1.00 | 30.16 | N |
| ATOM | 4330 | C | LYS | I | 117 | 66.601 | −35.198 | −15.806 | 1.00 | 23.83 | C |
| ATOM | 4331 | O | LYS | I | 117 | 65.761 | −34.306 | −15.768 | 1.00 | 23.81 | O |
| ATOM | 4332 | N | GLY | I | 118 | 67.567 | −35.325 | −14.898 | 1.00 | 23.55 | N |
| ATOM | 4333 | CA | GLY | I | 118 | 67.755 | −34.353 | −13.823 | 1.00 | 23.33 | C |
| ATOM | 4334 | C | GLY | I | 118 | 68.337 | −33.028 | −14.293 | 1.00 | 23.07 | C |
| ATOM | 4335 | O | GLY | I | 118 | 69.137 | −32.995 | −15.241 | 1.00 | 23.26 | O |
| ATOM | 4336 | N | PRO | I | 119 | 67.967 | −31.924 | −13.613 | 1.00 | 22.56 | N |
| ATOM | 4337 | CA | PRO | I | 119 | 68.368 | −30.601 | −14.074 | 1.00 | 22.20 | C |
| ATOM | 4338 | CB | PRO | I | 119 | 67.478 | −29.672 | −13.253 | 1.00 | 22.14 | C |
| ATOM | 4339 | CG | PRO | I | 119 | 67.287 | −30.384 | −11.974 | 1.00 | 22.21 | C |
| ATOM | 4340 | CD | PRO | I | 119 | 67.235 | −31.858 | −12.332 | 1.00 | 22.46 | C |
| ATOM | 4341 | C | PRO | I | 119 | 69.832 | −30.276 | −13.800 | 1.00 | 22.19 | C |
| ATOM | 4342 | O | PRO | I | 119 | 70.479 | −30.911 | −12.948 | 1.00 | 22.27 | O |
| ATOM | 4343 | N | SER | I | 120 | 70.342 | −29.301 | −14.536 | 1.00 | 21.77 | N |
| ATOM | 4344 | CA | SER | I | 120 | 71.617 | −28.666 | −14.214 | 1.00 | 21.86 | C |
| ATOM | 4345 | CB | SER | I | 120 | 72.435 | −28.457 | −15.484 | 1.00 | 21.98 | C |
| ATOM | 4346 | OG | SER | I | 120 | 72.901 | −29.699 | −15.965 | 1.00 | 24.34 | O |
| ATOM | 4347 | C | SER | I | 120 | 71.292 | −27.329 | −13.555 | 1.00 | 20.96 | C |
| ATOM | 4348 | O | SER | I | 120 | 70.424 | −26.603 | −14.034 | 1.00 | 21.32 | O |
| ATOM | 4349 | N | VAL | I | 121 | 71.957 | −27.012 | −12.450 | 1.00 | 19.63 | N |
| ATOM | 4350 | CA | VAL | I | 121 | 71.640 | −25.772 | −11.730 | 1.00 | 18.88 | C |
| ATOM | 4351 | CB | VAL | I | 121 | 71.238 | −26.042 | −10.258 | 1.00 | 18.42 | C |
| ATOM | 4352 | CG1 | VAL | I | 121 | 70.817 | −24.752 | −9.561 | 1.00 | 19.00 | C |
| ATOM | 4353 | CG2 | VAL | I | 121 | 70.114 | −27.088 | −10.178 | 1.00 | 18.67 | C |
| ATOM | 4354 | C | VAL | I | 121 | 72.795 | −24.782 | −11.826 | 1.00 | 18.82 | C |
| ATOM | 4355 | O | VAL | I | 121 | 73.908 | −25.054 | −11.371 | 1.00 | 18.57 | O |
| ATOM | 4356 | N | PHE | I | 122 | 72.523 | −23.631 | −12.431 | 1.00 | 18.40 | N |
| ATOM | 4357 | CA | PHE | I | 122 | 73.544 | −22.607 | −12.622 | 1.00 | 18.26 | C |
| ATOM | 4358 | CB | PHE | I | 122 | 73.669 | −22.277 | −14.113 | 1.00 | 18.13 | C |
| ATOM | 4359 | CG | PHE | I | 122 | 73.952 | −23.472 | −14.979 | 1.00 | 17.57 | C |
| ATOM | 4360 | CD1 | PHE | I | 122 | 75.158 | −24.159 | −14.867 | 1.00 | 16.87 | C |
| ATOM | 4361 | CE1 | PHE | I | 122 | 75.433 | −25.263 | −15.661 | 1.00 | 16.41 | C |
| ATOM | 4362 | CZ | PHE | I | 122 | 74.494 | −25.681 | −16.601 | 1.00 | 18.06 | C |
| ATOM | 4363 | CE2 | PHE | I | 122 | 73.278 | −24.998 | −16.720 | 1.00 | 18.14 | C |
| ATOM | 4364 | CD2 | PHE | I | 122 | 73.017 | −23.899 | −15.921 | 1.00 | 17.67 | C |
| ATOM | 4365 | C | PHE | I | 122 | 73.221 | −21.342 | −11.825 | 1.00 | 18.24 | C |
| ATOM | 4366 | O | PHE | I | 122 | 72.051 | −20.972 | −11.710 | 1.00 | 18.45 | O |
| ATOM | 4367 | N | PRO | I | 123 | 74.253 | −20.678 | −11.262 | 1.00 | 18.30 | N |
| ATOM | 4368 | CA | PRO | I | 123 | 73.961 | −19.471 | −10.500 | 1.00 | 18.53 | C |
| ATOM | 4369 | CB | PRO | I | 123 | 75.282 | −19.184 | −9.783 | 1.00 | 18.54 | C |
| ATOM | 4370 | CG | PRO | I | 123 | 76.341 | −19.729 | −10.717 | 1.00 | 18.45 | C |
| ATOM | 4371 | CD | PRO | I | 123 | 75.704 | −20.965 | −11.317 | 1.00 | 18.21 | C |
| ATOM | 4372 | C | PRO | I | 123 | 73.616 | −18.297 | −11.415 | 1.00 | 18.99 | C |
| ATOM | 4373 | O | PRO | I | 123 | 74.143 | −18.205 | −12.529 | 1.00 | 18.84 | O |
| ATOM | 4374 | N | LEU | I | 124 | 72.721 | −17.435 | −10.940 | 1.00 | 19.51 | N |
| ATOM | 4375 | CA | LEU | I | 124 | 72.499 | −16.114 | −11.520 | 1.00 | 19.78 | C |
| ATOM | 4376 | CB | LEU | I | 124 | 71.009 | −15.880 | −11.776 | 1.00 | 19.97 | C |
| ATOM | 4377 | CG | LEU | I | 124 | 70.297 | −16.757 | −12.807 | 1.00 | 20.29 | C |
| ATOM | 4378 | CD1 | LEU | I | 124 | 68.779 | −16.689 | −12.630 | 1.00 | 18.83 | C |
| ATOM | 4379 | CD2 | LEU | I | 124 | 70.684 | −16.333 | −14.221 | 1.00 | 20.19 | C |
| ATOM | 4380 | C | LEU | I | 124 | 73.031 | −15.134 | −10.472 | 1.00 | 20.53 | C |
| ATOM | 4381 | O | LEU | I | 124 | 72.318 | −14.769 | −9.526 | 1.00 | 18.78 | O |
| ATOM | 4382 | N | ALA | I | 125 | 74.304 | −14.755 | −10.617 | 1.00 | 21.15 | N |
| ATOM | 4383 | CA | ALA | I | 125 | 75.018 | −14.033 | −9.557 | 1.00 | 22.60 | C |
| ATOM | 4384 | CB | ALA | I | 125 | 76.536 | −14.098 | −9.779 | 1.00 | 22.18 | C |
| ATOM | 4385 | C | ALA | I | 125 | 74.540 | −12.578 | −9.446 | 1.00 | 23.64 | C |
| ATOM | 4386 | O | ALA | I | 125 | 74.149 | −11.981 | −10.450 | 1.00 | 23.24 | O |
| ATOM | 4387 | N | PRO | I | 126 | 74.556 | −12.010 | −8.221 | 1.00 | 24.85 | N |
| ATOM | 4388 | CA | PRO | I | 126 | 74.242 | −10.581 | −8.088 | 1.00 | 26.06 | C |
| ATOM | 4389 | CB | PRO | I | 126 | 74.132 | −10.386 | −6.571 | 1.00 | 26.23 | C |
| ATOM | 4390 | CG | PRO | I | 126 | 75.012 | −11.470 | −5.982 | 1.00 | 25.59 | C |
| ATOM | 4391 | CD | PRO | I | 126 | 74.853 | −12.644 | −6.920 | 1.00 | 24.84 | C |
| ATOM | 4392 | C | PRO | I | 126 | 75.382 | −9.730 | −8.667 | 1.00 | 27.50 | C |
| ATOM | 4393 | O | PRO | I | 126 | 76.552 | −10.004 | −8.385 | 1.00 | 27.26 | O |
| ATOM | 4394 | N | SER | I | 127 | 75.050 | −8.728 | −9.485 | 1.00 | 29.17 | N |
| ATOM | 4395 | CA | SER | I | 127 | 76.088 | −7.879 | −10.102 | 1.00 | 30.96 | C |
| ATOM | 4396 | CB | SER | I | 127 | 75.517 | −7.038 | −11.261 | 1.00 | 31.01 | C |
| ATOM | 4397 | OG | SER | I | 127 | 75.327 | −5.681 | −10.891 | 1.00 | 31.71 | O |
| ATOM | 4398 | C | SER | I | 127 | 76.812 | −7.005 | −9.060 | 1.00 | 31.81 | C |
| ATOM | 4399 | O | SER | I | 127 | 76.408 | −6.953 | −7.893 | 1.00 | 31.76 | O |
| ATOM | 4400 | N | SER | I | 128 | 77.893 | −6.353 | −9.494 | 1.00 | 33.00 | N |
| ATOM | 4401 | CA | SER | I | 128 | 78.704 | −5.457 | −8.660 | 1.00 | 34.15 | C |
| ATOM | 4402 | CB | SER | I | 128 | 79.795 | −4.803 | −9.521 | 1.00 | 34.25 | C |
| ATOM | 4403 | OG | SER | I | 128 | 80.579 | −3.879 | −8.773 | 1.00 | 36.52 | O |
| ATOM | 4404 | C | SER | I | 128 | 77.876 | −4.380 | −7.946 | 1.00 | 34.32 | C |
| ATOM | 4405 | O | SER | I | 128 | 77.319 | −3.476 | −8.582 | 1.00 | 34.88 | O |
| ATOM | 4406 | N | GLY | I | 133 | 72.341 | 0.234 | −6.185 | 1.00 | 30.85 | N |
| ATOM | 4407 | CA | GLY | I | 133 | 71.011 | 0.328 | −5.583 | 1.00 | 30.98 | C |
| ATOM | 4408 | C | GLY | I | 133 | 71.044 | 0.169 | −4.066 | 1.00 | 30.86 | C |
| ATOM | 4409 | O | GLY | I | 133 | 72.064 | 0.468 | −3.428 | 1.00 | 31.46 | O |
| ATOM | 4410 | N | GLY | I | 134 | 69.922 | −0.279 | −3.493 | 1.00 | 30.02 | N |
| ATOM | 4411 | CA | GLY | I | 134 | 69.831 | −0.666 | −2.077 | 1.00 | 28.67 | C |
| ATOM | 4412 | C | GLY | I | 134 | 69.530 | −2.155 | −1.930 | 1.00 | 27.79 | C |
| ATOM | 4413 | O | GLY | I | 134 | 69.743 | −2.754 | −0.871 | 1.00 | 27.24 | O |
| ATOM | 4414 | N | THR | I | 135 | 69.023 | −2.751 | −3.006 | 1.00 | 26.91 | N |
| ATOM | 4415 | CA | THR | I | 135 | 68.744 | −4.183 | −3.047 | 1.00 | 26.21 | C |
| ATOM | 4416 | CB | THR | I | 135 | 67.227 | −4.478 | −2.947 | 1.00 | 26.65 | C |
| ATOM | 4417 | OG1 | THR | I | 135 | 66.547 | −3.791 | −3.999 | 1.00 | 27.43 | O |
| ATOM | 4418 | CG2 | THR | I | 135 | 66.651 | −4.036 | −1.580 | 1.00 | 26.36 | C |
| ATOM | 4419 | C | THR | I | 135 | 69.289 | −4.796 | −4.335 | 1.00 | 25.29 | C |
| ATOM | 4420 | O | THR | I | 135 | 69.315 | −4.141 | −5.382 | 1.00 | 25.62 | O |
| ATOM | 4421 | N | ALA | I | 136 | 69.710 | −6.055 | −4.251 | 1.00 | 23.61 | N |
| ATOM | 4422 | CA | ALA | I | 136 | 70.256 | −6.773 | −5.395 | 1.00 | 22.44 | C |
| ATOM | 4423 | CB | ALA | I | 136 | 71.649 | −7.287 | −5.073 | 1.00 | 22.38 | C |
| ATOM | 4424 | C | ALA | I | 136 | 69.348 | −7.933 | −5.773 | 1.00 | 21.62 | C |
| ATOM | 4425 | O | ALA | I | 136 | 68.575 | −8.428 | −4.944 | 1.00 | 21.23 | O |
| ATOM | 4426 | N | ALA | I | 137 | 69.436 | −8.371 | −7.023 | 1.00 | 19.92 | N |
| ATOM | 4427 | CA | ALA | I | 137 | 68.724 | −9.578 | −7.414 | 1.00 | 18.86 | C |
| ATOM | 4428 | CB | ALA | I | 137 | 67.875 | −9.324 | −8.637 | 1.00 | 18.24 | C |
| ATOM | 4429 | C | ALA | I | 137 | 69.733 | −10.697 | −7.663 | 1.00 | 18.38 | C |
| ATOM | 4430 | O | ALA | I | 137 | 70.812 | −10.471 | −8.212 | 1.00 | 17.58 | O |
| ATOM | 4431 | N | LEU | I | 138 | 69.393 | −11.896 | −7.220 | 1.00 | 17.65 | N |
| ATOM | 4432 | CA | LEU | I | 138 | 70.212 | −13.061 | −7.501 | 1.00 | 17.73 | C |
| ATOM | 4433 | CB | LEU | I | 138 | 71.218 | −13.316 | −6.359 | 1.00 | 18.06 | C |
| ATOM | 4434 | CG | LEU | I | 138 | 70.733 | −13.512 | −4.919 | 1.00 | 18.35 | C |
| ATOM | 4435 | CD1 | LEU | I | 138 | 70.392 | −14.979 | −4.674 | 1.00 | 19.40 | C |
| ATOM | 4436 | CD2 | LEU | I | 138 | 71.815 | −13.054 | −3.949 | 1.00 | 19.98 | C |
| ATOM | 4437 | C | LEU | I | 138 | 69.287 | −14.243 | −7.747 | 1.00 | 17.89 | C |
| ATOM | 4438 | O | LEU | I | 138 | 68.078 | −14.150 | −7.488 | 1.00 | 17.98 | O |
| ATOM | 4439 | N | GLY | I | 139 | 69.831 | −15.345 | −8.252 | 1.00 | 17.51 | N |
| ATOM | 4440 | CA | GLY | I | 139 | 68.996 | −16.508 | −8.520 | 1.00 | 17.74 | C |
| ATOM | 4441 | C | GLY | I | 139 | 69.707 | −17.770 | −8.945 | 1.00 | 18.12 | C |
| ATOM | 4442 | O | GLY | I | 139 | 70.928 | −17.887 | −8.829 | 1.00 | 17.65 | N |
| ATOM | 4443 | N | CYS | I | 140 | 68.913 | −18.733 | −9.405 | 1.00 | 18.82 | N |
| ATOM | 4444 | CA | CYS | I | 140 | 69.413 | −19.998 | −9.928 | 1.00 | 19.27 | C |
| ATOM | 4445 | CB | CYS | I | 140 | 69.203 | −21.126 | −8.931 | 1.00 | 19.51 | C |
| ATOM | 4446 | SG | CYS | I | 140 | 70.354 | −21.122 | −7.552 | 1.00 | 25.04 | S |
| ATOM | 4447 | C | CYS | I | 140 | 68.618 | −20.314 | −11.181 | 1.00 | 18.70 | C |
| ATOM | 4448 | O | CYS | I | 140 | 67.402 | −20.159 | −11.200 | 1.00 | 18.71 | O |
| ATOM | 4449 | N | LEU | I | 141 | 69.323 | −20.749 | −12.218 | 1.00 | 18.50 | N |
| ATOM | 4450 | CA | LEU | I | 141 | 68.699 | −21.205 | −13.441 | 1.00 | 17.95 | C |
| ATOM | 4451 | CB | LEU | I | 141 | 69.475 | −20.688 | −14.651 | 1.00 | 17.62 | C |
| ATOM | 4452 | CG | LEU | I | 141 | 69.114 | −21.209 | −16.044 | 1.00 | 18.61 | C |
| ATOM | 4453 | CD1 | LEU | I | 141 | 67.730 | −20.789 | −16.451 | 1.00 | 17.64 | C |
| ATOM | 4454 | CD2 | LEU | I | 141 | 70.129 | −20.681 | −17.020 | 1.00 | 18.51 | C |
| ATOM | 4455 | C | LEU | I | 141 | 68.715 | −22.721 | −13.375 | 1.00 | 18.20 | C |
| ATOM | 4456 | O | LEU | I | 141 | 69.787 | −23.340 | −13.251 | 1.00 | 17.64 | O |
| ATOM | 4457 | N | VAL | I | 142 | 67.524 | −23.310 | −13.420 | 1.00 | 17.93 | N |
| ATOM | 4458 | CA | VAL | I | 142 | 67.364 | −24.754 | −13.330 | 1.00 | 18.74 | C |
| ATOM | 4459 | CB | VAL | I | 142 | 66.199 | −25.148 | −12.362 | 1.00 | 18.72 | C |
| ATOM | 4460 | CG1 | VAL | I | 142 | 66.062 | −26.677 | −12.250 | 1.00 | 19.84 | C |
| ATOM | 4461 | CG2 | VAL | I | 142 | 66.437 | −24.550 | −10.992 | 1.00 | 19.31 | C |
| ATOM | 4462 | C | VAL | I | 142 | 67.084 | −25.227 | −14.747 | 1.00 | 18.89 | C |
| ATOM | 4463 | O | VAL | I | 142 | 65.979 | −25.050 | −15.253 | 1.00 | 19.55 | O |
| ATOM | 4464 | N | LYS | I | 143 | 68.087 | −25.826 | −15.379 | 1.00 | 18.70 | N |
| ATOM | 4465 | CA | LYS | I | 143 | 68.007 | −26.085 | −16.809 | 1.00 | 19.44 | C |
| ATOM | 4466 | CB | LYS | I | 143 | 69.147 | −25.362 | −17.533 | 1.00 | 18.92 | C |
| ATOM | 4467 | CG | LYS | I | 143 | 68.881 | −25.187 | −19.009 | 1.00 | 20.84 | C |
| ATOM | 4468 | CD | LYS | I | 143 | 69.679 | −24.053 | −19.593 | 1.00 | 22.40 | C |
| ATOM | 4469 | CE | LYS | I | 143 | 70.163 | −24.399 | −20.995 | 1.00 | 23.43 | C |
| ATOM | 4470 | NZ | LYS | I | 143 | 69.024 | −24.479 | −21.940 | 1.00 | 23.63 | N |
| ATOM | 4471 | C | LYS | I | 143 | 67.994 | −27.561 | −17.206 | 1.00 | 18.99 | C |
| ATOM | 4472 | O | LYS | I | 143 | 68.653 | −28.398 | −16.580 | 1.00 | 19.88 | O |
| ATOM | 4473 | N | ASP | I | 144 | 67.237 | −27.843 | −18.258 | 1.00 | 18.31 | N |
| ATOM | 4474 | CA | ASP | I | 144 | 67.293 | −29.105 | −19.011 | 1.00 | 18.55 | C |
| ATOM | 4475 | CB | ASP | I | 144 | 68.693 | −29.324 | −19.628 | 1.00 | 18.31 | C |
| ATOM | 4476 | CG | ASP | I | 144 | 69.034 | −28.305 | −20.697 | 1.00 | 19.43 | C |
| ATOM | 4477 | OD1 | ASP | I | 144 | 68.107 | −27.657 | −21.237 | 1.00 | 20.95 | O |
| ATOM | 4478 | OD2 | ASP | I | 144 | 70.240 | −28.159 | −21.005 | 1.00 | 18.60 | O |
| ATOM | 4479 | C | ASP | I | 144 | 66.872 | −30.325 | −18.208 | 1.00 | 18.07 | C |
| ATOM | 4480 | O | ASP | I | 144 | 67.639 | −31.276 | −18.062 | 1.00 | 18.58 | O |
| ATOM | 4481 | N | TYR | I | 145 | 65.655 | −30.312 | −17.687 | 1.00 | 17.97 | N |
| ATOM | 4482 | CA | TYR | I | 145 | 65.154 | −31.473 | −16.962 | 1.00 | 18.01 | C |
| ATOM | 4483 | CB | TYR | I | 145 | 64.969 | −31.159 | −15.472 | 1.00 | 18.17 | C |
| ATOM | 4484 | CG | TYR | I | 145 | 63.916 | −30.095 | −15.206 | 1.00 | 18.94 | C |
| ATOM | 4485 | CD1 | TYR | I | 145 | 64.238 | −28.732 | −15.266 | 1.00 | 19.80 | C |
| ATOM | 4486 | CE1 | TYR | I | 145 | 63.268 | −27.757 | −15.031 | 1.00 | 21.09 | C |
| ATOM | 4487 | CZ | TYR | I | 145 | 61.966 | −28.140 | −14.740 | 1.00 | 21.09 | C |
| ATOM | 4488 | OH | TYR | I | 145 | 61.007 | −27.186 | −14.505 | 1.00 | 19.65 | O |
| ATOM | 4489 | CE2 | TYR | I | 145 | 61.619 | −29.477 | −14.685 | 1.00 | 21.22 | C |
| ATOM | 4490 | CD2 | TYR | I | 145 | 62.602 | −30.450 | −14.911 | 1.00 | 19.75 | C |
| ATOM | 4491 | C | TYR | I | 145 | 63.838 | −31.954 | −17.566 | 1.00 | 18.37 | C |
| ATOM | 4492 | O | TYR | I | 145 | 63.200 | −31.247 | −18.350 | 1.00 | 17.52 | O |
| ATOM | 4493 | N | PHE | I | 146 | 63.448 | −33.163 | −17.178 | 1.00 | 18.36 | N |
| ATOM | 4494 | CA | PHE | I | 146 | 62.194 | −33.771 | −17.595 | 1.00 | 18.65 | C |
| ATOM | 4495 | CB | PHE | I | 146 | 62.269 | −34.254 | −19.054 | 1.00 | 17.80 | C |
| ATOM | 4496 | CG | PHE | I | 146 | 60.979 | −34.841 | −19.555 | 1.00 | 18.07 | C |
| ATOM | 4497 | CD1 | PHE | I | 146 | 60.698 | −36.200 | −19.369 | 1.00 | 18.56 | C |
| ATOM | 4498 | CE1 | PHE | I | 146 | 59.475 | −36.752 | −19.812 | 1.00 | 18.70 | C |
| ATOM | 4499 | CZ | PHE | I | 146 | 58.542 | −35.931 | −20.453 | 1.00 | 17.06 | C |
| ATOM | 4500 | CE2 | PHE | I | 146 | 58.831 | −34.571 | −20.644 | 1.00 | 16.59 | C |
| ATOM | 4501 | CD2 | PHE | I | 146 | 60.039 | −34.041 | −20.196 | 1.00 | 15.75 | C |
| ATOM | 4502 | C | PHE | I | 146 | 61.893 | −34.941 | −16.655 | 1.00 | 19.02 | C |
| ATOM | 4503 | O | PHE | I | 146 | 62.801 | −35.676 | −16.268 | 1.00 | 19.38 | O |
| ATOM | 4504 | N | PRO | I | 147 | 60.625 | −35.100 | −16.257 | 1.00 | 19.81 | N |
| ATOM | 4505 | CA | PRO | I | 147 | 59.546 | −34.176 | −16.578 | 1.00 | 20.38 | C |
| ATOM | 4506 | CB | PRO | I | 147 | 58.320 | −35.094 | −16.605 | 1.00 | 19.82 | C |
| ATOM | 4507 | CG | PRO | I | 147 | 58.629 | −36.138 | −15.590 | 1.00 | 21.11 | C |
| ATOM | 4508 | CD | PRO | I | 147 | 60.135 | −36.280 | −15.518 | 1.00 | 19.91 | C |
| ATOM | 4509 | C | PRO | I | 147 | 59.417 | −33.121 | −15.492 | 1.00 | 21.07 | C |
| ATOM | 4510 | O | PRO | I | 147 | 60.266 | −33.055 | −14.581 | 1.00 | 20.72 | O |
| ATOM | 4511 | N | GLU | I | 148 | 58.369 | −32.308 | −15.594 | 1.00 | 22.21 | N |
| ATOM | 4512 | CA | GLU | I | 148 | 57.894 | −31.500 | −14.475 | 1.00 | 23.34 | C |
| ATOM | 4513 | CB | GLU | I | 148 | 56.716 | −30.652 | −14.941 | 1.00 | 23.87 | C |
| ATOM | 4514 | CG | GLU | I | 148 | 57.136 | −29.497 | −15.840 | 1.00 | 24.62 | C |
| ATOM | 4515 | CD | GLU | I | 148 | 57.347 | −28.227 | −15.053 | 1.00 | 27.80 | C |
| ATOM | 4516 | OE1 | GLU | I | 148 | 58.336 | −28.138 | −14.269 | 1.00 | 28.64 | O |
| ATOM | 4517 | OE2 | GLU | I | 148 | 56.498 | −27.322 | −15.200 | 1.00 | 27.73 | O |
| ATOM | 4518 | C | GLU | I | 148 | 57.465 | −32.431 | −13.343 | 1.00 | 23.83 | C |
| ATOM | 4519 | O | GLU | I | 148 | 57.148 | −33.581 | −13.600 | 1.00 | 23.76 | O |
| ATOM | 4520 | N | PRO | I | 149 | 57.474 | −31.953 | −12.079 | 1.00 | 24.56 | N |
| ATOM | 4521 | CA | PRO | I | 149 | 57.910 | −30.642 | −11.629 | 1.00 | 24.59 | C |
| ATOM | 4522 | CB | PRO | I | 149 | 56.848 | −30.265 | −10.587 | 1.00 | 24.65 | C |
| ATOM | 4523 | CG | PRO | I | 149 | 56.497 | −31.599 | −9.948 | 1.00 | 25.21 | C |
| ATOM | 4524 | CD | PRO | I | 149 | 56.771 | −32.683 | −11.003 | 1.00 | 24.49 | C |
| ATOM | 4525 | C | PRO | I | 149 | 59.258 | −30.672 | −10.948 | 1.00 | 25.03 | C |
| ATOM | 4526 | O | PRO | I | 149 | 59.774 | −31.749 | −10.581 | 1.00 | 25.06 | O |
| ATOM | 4527 | N | VAL | I | 150 | 59.818 | −29.479 | −10.793 | 1.00 | 24.81 | N |
| ATOM | 4528 | CA | VAL | I | 150 | 60.978 | −29.271 | −9.955 | 1.00 | 25.56 | C |
| ATOM | 4529 | CB | VAL | I | 150 | 62.172 | −28.722 | −10.796 | 1.00 | 25.91 | C |
| ATOM | 4530 | CG1 | VAL | I | 150 | 62.032 | −27.232 | −11.086 | 1.00 | 25.92 | C |
| ATOM | 4531 | CG2 | VAL | I | 150 | 63.473 | −29.023 | −10.133 | 1.00 | 26.82 | C |
| ATOM | 4532 | C | VAL | I | 150 | 60.521 | −28.338 | −8.823 | 1.00 | 25.45 | C |
| ATOM | 4533 | O | VAL | I | 150 | 59.593 | −27.553 | −9.012 | 1.00 | 25.15 | O |
| ATOM | 4534 | N | THR | I | 151 | 61.110 | −28.477 | −7.639 | 1.00 | 25.11 | N |
| ATOM | 4535 | CA | THR | I | 151 | 60.850 | −27.524 | −6.562 | 1.00 | 25.20 | C |
| ATOM | 4536 | CB | THR | I | 151 | 60.297 | −28.211 | −5.290 | 1.00 | 25.17 | C |
| ATOM | 4537 | OG1 | THR | I | 151 | 61.237 | −29.189 | −4.821 | 1.00 | 27.11 | O |
| ATOM | 4538 | CG2 | THR | I | 151 | 58.939 | −28.869 | −5.572 | 1.00 | 25.25 | C |
| ATOM | 4539 | C | THR | I | 151 | 62.128 | −26.757 | −6.224 | 1.00 | 24.38 | C |
| ATOM | 4540 | O | THR | I | 151 | 63.223 | −27.299 | −6.313 | 1.00 | 24.68 | O |
| ATOM | 4541 | N | VAL | I | 152 | 61.981 | −25.489 | −5.863 | 1.00 | 23.44 | N |
| ATOM | 4542 | CA | VAL | I | 152 | 63.113 | −24.655 | −5.487 | 1.00 | 22.31 | C |
| ATOM | 4543 | CB | VAL | I | 152 | 63.393 | −23.557 | −6.530 | 1.00 | 22.24 | C |
| ATOM | 4544 | CG1 | VAL | I | 152 | 64.674 | −22.785 | −6.168 | 1.00 | 22.49 | C |
| ATOM | 4545 | CG2 | VAL | I | 152 | 63.529 | −24.154 | −7.924 | 1.00 | 22.53 | C |
| ATOM | 4546 | C | VAL | I | 152 | 62.797 | −23.999 | −4.149 | 1.00 | 21.94 | C |
| ATOM | 4547 | O | VAL | I | 152 | 61.698 | −23.495 | −3.952 | 1.00 | 22.10 | O |
| ATOM | 4548 | N | SER | I | 153 | 63.742 | −24.049 | −3.222 | 1.00 | 20.92 | N |
| ATOM | 4549 | CA | SER | I | 153 | 63.650 | −23.255 | −2.009 | 1.00 | 20.61 | C |
| ATOM | 4550 | CB | SER | I | 153 | 63.415 | −24.136 | −0.786 | 1.00 | 20.57 | C |
| ATOM | 4551 | OG | SER | I | 153 | 64.519 | −24.985 | −0.576 | 1.00 | 21.34 | O |
| ATOM | 4552 | C | SER | I | 153 | 64.946 | −22.476 | −1.877 | 1.00 | 19.86 | C |
| ATOM | 4553 | O | SER | I | 153 | 65.927 | −22.771 | −2.575 | 1.00 | 20.13 | O |
| ATOM | 4554 | N | TRP | I | 154 | 64.938 | −21.483 | −0.992 | 1.00 | 18.37 | N |
| ATOM | 4555 | CA | TRP | I | 154 | 66.107 | −20.674 | −0.712 | 1.00 | 17.74 | C |
| ATOM | 4556 | CB | TRP | I | 154 | 65.830 | −19.216 | −1.082 | 1.00 | 17.37 | C |
| ATOM | 4557 | CG | TRP | I | 154 | 65.872 | −19.046 | −2.561 | 1.00 | 17.66 | C |
| ATOM | 4558 | CD1 | TRP | I | 154 | 64.822 | −19.159 | −3.437 | 1.00 | 17.64 | C |
| ATOM | 4559 | NE1 | TRP | I | 154 | 65.265 | −18.973 | −4.727 | 1.00 | 17.36 | N |
| ATOM | 4560 | CE2 | TRP | I | 154 | 66.621 | −18.745 | −4.703 | 1.00 | 17.65 | C |
| ATOM | 4561 | CD2 | TRP | I | 154 | 67.039 | −18.801 | −3.353 | 1.00 | 16.56 | C |
| ATOM | 4562 | CE3 | TRP | I | 154 | 68.399 | −18.612 | −3.050 | 1.00 | 17.87 | C |
| ATOM | 4563 | CZ3 | TRP | I | 154 | 69.305 | −18.372 | −4.101 | 1.00 | 17.53 | C |
| ATOM | 4564 | CH2 | TRP | I | 154 | 68.850 | −18.326 | −5.447 | 1.00 | 17.13 | C |
| ATOM | 4565 | CZ2 | TRP | I | 154 | 67.519 | −18.513 | −5.760 | 1.00 | 18.27 | C |
| ATOM | 4566 | C | TRP | I | 154 | 66.478 | −20.812 | 0.750 | 1.00 | 17.45 | C |
| ATOM | 4567 | O | TRP | I | 154 | 65.617 | −20.693 | 1.637 | 1.00 | 17.10 | O |
| ATOM | 4568 | N | ASN | I | 155 | 67.753 | −21.095 | 0.991 | 1.00 | 16.88 | N |
| ATOM | 4569 | CA | ASN | I | 155 | 68.256 | −21.356 | 2.332 | 1.00 | 17.56 | C |
| ATOM | 4570 | CB | ASN | I | 155 | 68.485 | −20.042 | 3.077 | 1.00 | 17.00 | C |
| ATOM | 4571 | CG | ASN | I | 155 | 69.559 | −19.195 | 2.429 | 1.00 | 17.66 | C |
| ATOM | 4572 | OD1 | ASN | I | 155 | 70.308 | −19.671 | 1.572 | 1.00 | 16.48 | O |
| ATOM | 4573 | ND2 | ASN | I | 155 | 69.652 | −17.932 | 2.846 | 1.00 | 16.01 | N |
| ATOM | 4574 | C | ASN | I | 155 | 67.387 | −22.330 | 3.138 | 1.00 | 17.96 | C |
| ATOM | 4575 | O | ASN | I | 155 | 67.031 | −22.071 | 4.292 | 1.00 | 17.84 | O |
| ATOM | 4576 | N | SER | I | 156 | 67.062 | −23.443 | 2.487 | 1.00 | 19.17 | N |
| ATOM | 4577 | CA | SER | I | 156 | 66.260 | −24.534 | 3.030 | 1.00 | 19.87 | C |
| ATOM | 4578 | CB | SER | I | 156 | 67.040 | −25.289 | 4.122 | 1.00 | 20.36 | C |
| ATOM | 4579 | OG | SER | I | 156 | 68.313 | −25.701 | 3.636 | 1.00 | 20.42 | O |
| ATOM | 4580 | C | SER | I | 156 | 64.881 | −24.085 | 3.520 | 1.00 | 20.45 | C |
| ATOM | 4581 | O | SER | I | 156 | 64.398 | −24.564 | 4.557 | 1.00 | 20.47 | O |
| ATOM | 4582 | N | GLY | I | 157 | 64.257 | −23.168 | 2.766 | 1.00 | 20.17 | N |
| ATOM | 4583 | CA | GLY | I | 157 | 62.919 | −22.656 | 3.086 | 1.00 | 19.27 | C |
| ATOM | 4584 | C | GLY | I | 157 | 62.898 | −21.516 | 4.096 | 1.00 | 19.02 | C |
| ATOM | 4585 | O | GLY | I | 157 | 61.838 | −20.937 | 4.344 | 1.00 | 19.11 | O |
| ATOM | 4586 | N | ALA | I | 158 | 64.054 | −21.181 | 4.674 | 1.00 | 18.51 | N |
| ATOM | 4587 | CA | ALA | I | 158 | 64.144 | −20.096 | 5.660 | 1.00 | 17.86 | C |
| ATOM | 4588 | CB | ALA | I | 158 | 65.458 | −20.163 | 6.451 | 1.00 | 18.22 | C |
| ATOM | 4589 | C | ALA | I | 158 | 63.975 | −18.714 | 5.020 | 1.00 | 17.56 | C |
| ATOM | 4590 | O | ALA | I | 158 | 63.740 | −17.736 | 5.718 | 1.00 | 17.05 | O |
| ATOM | 4591 | N | LEU | I | 159 | 64.104 | −18.645 | 3.694 | 1.00 | 16.65 | N |
| ATOM | 4592 | CA | LEU | I | 159 | 63.961 | −17.381 | 2.959 | 1.00 | 16.51 | C |
| ATOM | 4593 | CB | LEU | I | 159 | 65.281 | −17.021 | 2.261 | 1.00 | 16.07 | C |
| ATOM | 4594 | CG | LEU | I | 159 | 65.331 | −15.801 | 1.326 | 1.00 | 17.37 | C |
| ATOM | 4595 | CD1 | LEU | I | 159 | 65.078 | −14.474 | 2.047 | 1.00 | 17.84 | C |
| ATOM | 4596 | CD2 | LEU | I | 159 | 66.686 | −15.744 | 0.654 | 1.00 | 17.49 | C |
| ATOM | 4597 | C | LEU | I | 159 | 62.826 | −17.467 | 1.934 | 1.00 | 15.81 | C |
| ATOM | 4598 | O | LEU | I | 159 | 62.928 | −18.208 | 0.956 | 1.00 | 16.15 | O |
| ATOM | 4599 | N | THR | I | 160 | 61.744 | −16.728 | 2.171 | 1.00 | 15.84 | N |
| ATOM | 4600 | CA | THR | I | 160 | 60.585 | −16.721 | 1.260 | 1.00 | 16.08 | C |
| ATOM | 4601 | CB | THR | I | 160 | 59.287 | −17.255 | 1.922 | 1.00 | 15.66 | C |
| ATOM | 4602 | OG1 | THR | I | 160 | 58.989 | −16.476 | 3.084 | 1.00 | 13.79 | O |
| ATOM | 4603 | CG2 | THR | I | 160 | 59.423 | −18.739 | 2.295 | 1.00 | 15.38 | C |
| ATOM | 4604 | C | THR | I | 160 | 60.282 | −15.325 | 0.713 | 1.00 | 16.59 | C |
| ATOM | 4605 | O | THR | I | 160 | 59.756 | −15.207 | −0.396 | 1.00 | 16.07 | O |
| ATOM | 4606 | N | SER | I | 161 | 60.597 | −14.297 | 1.502 | 1.00 | 16.95 | N |
| ATOM | 4607 | CA | SER | I | 161 | 60.339 | −12.901 | 1.129 | 1.00 | 18.31 | C |
| ATOM | 4608 | CB | SER | I | 161 | 60.709 | −11.939 | 2.260 | 1.00 | 18.62 | C |
| ATOM | 4609 | OG | SER | I | 161 | 59.954 | −12.219 | 3.419 | 1.00 | 21.67 | O |
| ATOM | 4610 | C | SER | I | 161 | 61.144 | −12.528 | −0.105 | 1.00 | 18.15 | C |
| ATOM | 4611 | O | SER | I | 161 | 62.347 | −12.750 | −0.150 | 1.00 | 17.69 | O |
| ATOM | 4612 | N | GLY | I | 162 | 60.457 | −11.991 | −1.109 | 1.00 | 18.58 | N |
| ATOM | 4613 | CA | GLY | I | 162 | 61.137 | −11.456 | −2.290 | 1.00 | 18.65 | C |
| ATOM | 4614 | C | GLY | I | 162 | 61.509 | −12.541 | −3.276 | 1.00 | 18.30 | C |
| ATOM | 4615 | O | GLY | I | 162 | 62.180 | −12.267 | −4.275 | 1.00 | 18.01 | O |
| ATOM | 4616 | N | VAL | I | 163 | 61.076 | −13.773 | −3.000 | 1.00 | 17.68 | N |
| ATOM | 4617 | CA | VAL | I | 163 | 61.338 | −14.898 | −3.898 | 1.00 | 18.05 | C |
| ATOM | 4618 | CB | VAL | I | 163 | 61.353 | −16.247 | −3.135 | 1.00 | 18.03 | C |
| ATOM | 4619 | CG1 | VAL | I | 163 | 61.438 | −17.453 | −4.115 | 1.00 | 20.22 | C |
| ATOM | 4620 | CG2 | VAL | I | 163 | 62.502 | −16.277 | −2.143 | 1.00 | 18.22 | C |
| ATOM | 4621 | C | VAL | I | 163 | 60.298 | −14.949 | −5.017 | 1.00 | 17.73 | C |
| ATOM | 4622 | O | VAL | I | 163 | 59.099 | −14.834 | −4.765 | 1.00 | 17.87 | O |
| ATOM | 4623 | N | HIS | I | 164 | 60.757 | −15.117 | −6.253 | 1.00 | 17.10 | N |
| ATOM | 4624 | CA | HIS | I | 164 | 59.860 | −15.428 | −7.359 | 1.00 | 16.90 | C |
| ATOM | 4625 | CB | HIS | I | 164 | 59.780 | −14.265 | −8.341 | 1.00 | 16.65 | C |
| ATOM | 4626 | CG | HIS | I | 164 | 59.157 | −13.027 | −7.772 | 1.00 | 17.21 | C |
| ATOM | 4627 | ND1 | HIS | I | 164 | 57.860 | −12.991 | −7.309 | 1.00 | 18.63 | N |
| ATOM | 4628 | CE1 | HIS | I | 164 | 57.578 | −11.769 | −6.889 | 1.00 | 19.23 | C |
| ATOM | 4629 | NE2 | HIS | I | 164 | 58.642 | −11.010 | −7.080 | 1.00 | 19.67 | N |
| ATOM | 4630 | CD2 | HIS | I | 164 | 59.638 | −11.769 | −7.644 | 1.00 | 17.57 | C |
| ATOM | 4631 | C | HIS | I | 164 | 60.382 | −16.641 | −8.105 | 1.00 | 17.37 | C |
| ATOM | 4632 | O | HIS | I | 164 | 61.461 | −16.583 | −8.706 | 1.00 | 17.00 | O |
| ATOM | 4633 | N | THR | I | 165 | 59.619 | −17.731 | −8.091 | 1.00 | 17.30 | N |
| ATOM | 4634 | CA | THR | I | 165 | 59.997 | −18.900 | −8.864 | 1.00 | 18.14 | C |
| ATOM | 4635 | CB | THR | I | 165 | 59.984 | −20.164 | −8.004 | 1.00 | 17.98 | C |
| ATOM | 4636 | OG1 | THR | I | 165 | 60.930 | −20.003 | −6.935 | 1.00 | 18.52 | O |
| ATOM | 4637 | CG2 | THR | I | 165 | 60.349 | −21.410 | −8.832 | 1.00 | 19.31 | C |
| ATOM | 4638 | C | THR | I | 165 | 59.054 | −18.967 | −10.064 | 1.00 | 18.29 | C |
| ATOM | 4639 | O | THR | I | 165 | 57.843 | −19.107 | −9.903 | 1.00 | 18.74 | O |
| ATOM | 4640 | N | PHE | I | 166 | 59.621 | −18.824 | −11.255 | 1.00 | 17.89 | N |
| ATOM | 4641 | CA | PHE | I | 166 | 58.834 | −18.695 | −12.478 | 1.00 | 18.63 | C |
| ATOM | 4642 | CB | PHE | I | 166 | 59.686 | −18.028 | −13.561 | 1.00 | 18.42 | C |
| ATOM | 4643 | CG | PHE | I | 166 | 60.013 | −16.611 | −13.247 | 1.00 | 18.07 | C |
| ATOM | 4644 | CD1 | PHE | I | 166 | 59.119 | −15.598 | −13.568 | 1.00 | 17.32 | C |
| ATOM | 4645 | CE1 | PHE | I | 166 | 59.407 | −14.277 | −13.246 | 1.00 | 16.11 | C |
| ATOM | 4646 | CZ | PHE | I | 166 | 60.585 | −13.954 | −12.596 | 1.00 | 17.39 | C |
| ATOM | 4647 | CE2 | PHE | I | 166 | 61.490 | −14.958 | −12.258 | 1.00 | 18.79 | C |
| ATOM | 4648 | CD2 | PHE | I | 166 | 61.195 | −16.285 | −12.575 | 1.00 | 16.95 | C |
| ATOM | 4649 | C | PHE | I | 166 | 58.309 | −20.035 | −12.967 | 1.00 | 19.22 | C |
| ATOM | 4650 | O | PHE | I | 166 | 58.932 | −21.052 | −12.683 | 1.00 | 18.61 | O |
| ATOM | 4651 | N | PRO | I | 167 | 57.164 | −20.033 | −13.702 | 1.00 | 20.04 | N |
| ATOM | 4652 | CA | PRO | I | 167 | 56.680 | −21.246 | −14.352 | 1.00 | 20.29 | C |
| ATOM | 4653 | CB | PRO | I | 167 | 55.485 | −20.768 | −15.183 | 1.00 | 20.60 | C |
| ATOM | 4654 | CG | PRO | I | 167 | 55.019 | −19.516 | −14.510 | 1.00 | 21.18 | C |
| ATOM | 4655 | CD | PRO | I | 167 | 56.255 | −18.889 | −13.918 | 1.00 | 20.22 | C |
| ATOM | 4656 | C | PRO | I | 167 | 57.754 | −21.750 | −15.282 | 1.00 | 20.48 | C |
| ATOM | 4657 | O | PRO | I | 167 | 58.469 | −20.949 | −15.900 | 1.00 | 20.41 | O |
| ATOM | 4658 | N | ALA | I | 168 | 57.865 | −23.066 | −15.394 | 1.00 | 20.79 | N |
| ATOM | 4659 | CA | ALA | I | 168 | 58.852 | −23.661 | −16.300 | 1.00 | 21.20 | C |
| ATOM | 4660 | CB | ALA | I | 168 | 58.914 | −25.157 | −16.086 | 1.00 | 21.54 | C |
| ATOM | 4661 | C | ALA | I | 168 | 58.476 | −23.356 | −17.744 | 1.00 | 21.53 | C |
| ATOM | 4662 | O | ALA | I | 168 | 57.296 | −23.187 | −18.049 | 1.00 | 21.00 | O |
| ATOM | 4663 | N | VAL | I | 169 | 59.476 | −23.254 | −18.621 | 1.00 | 21.21 | N |
| ATOM | 4664 | CA | VAL | I | 169 | 59.212 | −23.187 | −20.054 | 1.00 | 21.49 | C |
| ATOM | 4665 | CB | VAL | I | 169 | 59.836 | −21.926 | −20.725 | 1.00 | 21.74 | C |
| ATOM | 4666 | CG1 | VAL | I | 169 | 59.411 | −20.667 | −19.976 | 1.00 | 22.55 | C |
| ATOM | 4667 | CG2 | VAL | I | 169 | 61.357 | −22.011 | −20.788 | 1.00 | 21.38 | C |
| ATOM | 4668 | C | VAL | I | 169 | 59.723 | −24.467 | −20.716 | 1.00 | 21.45 | C |
| ATOM | 4669 | O | VAL | I | 169 | 60.747 | −25.034 | −20.298 | 1.00 | 20.44 | O |
| ATOM | 4670 | N | LEU | I | 170 | 58.998 | −24.913 | −21.731 | 1.00 | 21.04 | N |
| ATOM | 4671 | CA | LEU | I | 170 | 59.376 | −26.090 | −22.484 | 1.00 | 21.91 | C |
| ATOM | 4672 | CB | LEU | I | 170 | 58.128 | −26.836 | −22.972 | 1.00 | 22.34 | C |
| ATOM | 4673 | CG | LEU | I | 170 | 58.354 | −28.118 | −23.784 | 1.00 | 23.81 | C |
| ATOM | 4674 | CD1 | LEU | I | 170 | 59.005 | −29.214 | −22.910 | 1.00 | 24.14 | C |
| ATOM | 4675 | CD2 | LEU | I | 170 | 57.030 | −28.584 | −24.353 | 1.00 | 23.60 | C |
| ATOM | 4676 | C | LEU | I | 170 | 60.253 | −25.671 | −23.662 | 1.00 | 21.99 | C |
| ATOM | 4677 | O | LEU | I | 170 | 59.826 | −24.914 | −24.528 | 1.00 | 21.80 | O |
| ATOM | 4678 | N | GLN | I | 171 | 61.485 | −26.162 | −23.683 | 1.00 | 21.80 | N |
| ATOM | 4679 | CA | GLN | I | 171 | 62.405 | −25.815 | −24.756 | 1.00 | 21.98 | C |
| ATOM | 4680 | CB | GLN | I | 171 | 63.848 | −25.922 | −24.256 | 1.00 | 21.84 | C |
| ATOM | 4681 | CG | GLN | I | 171 | 64.086 | −25.113 | −22.974 | 1.00 | 22.26 | C |
| ATOM | 4682 | CD | GLN | I | 171 | 65.423 | −25.400 | −22.327 | 1.00 | 24.64 | C |
| ATOM | 4683 | OE1 | GLN | I | 171 | 66.333 | −24.566 | −22.370 | 1.00 | 24.97 | O |
| ATOM | 4684 | NE2 | GLN | I | 171 | 65.564 | −26.587 | −21.740 | 1.00 | 23.29 | N |
| ATOM | 4685 | C | GLN | I | 171 | 62.145 | −26.691 | −25.994 | 1.00 | 22.27 | C |
| ATOM | 4686 | O | GLN | I | 171 | 61.480 | −27.731 | −25.902 | 1.00 | 22.03 | O |
| ATOM | 4687 | N | SER | I | 172 | 62.645 | −26.259 | −27.149 | 1.00 | 22.83 | N |
| ATOM | 4688 | CA | SER | I | 172 | 62.496 | −27.037 | −28.385 | 1.00 | 23.10 | C |
| ATOM | 4689 | CB | SER | I | 172 | 62.987 | −26.238 | −29.596 | 1.00 | 23.13 | C |
| ATOM | 4690 | OG | SER | I | 172 | 64.394 | −26.083 | −29.560 | 1.00 | 23.79 | O |
| ATOM | 4691 | C | SER | I | 172 | 63.203 | −28.404 | −28.294 | 1.00 | 22.92 | C |
| ATOM | 4692 | O | SER | I | 172 | 62.860 | −29.324 | −29.028 | 1.00 | 23.88 | O |
| ATOM | 4693 | N | SER | I | 173 | 64.158 | −28.539 | −27.372 | 1.00 | 22.44 | N |
| ATOM | 4694 | CA | SER | I | 173 | 64.842 | −29.824 | −27.113 | 1.00 | 21.76 | C |
| ATOM | 4695 | CB | SER | I | 173 | 66.082 | −29.610 | −26.243 | 1.00 | 22.09 | C |
| ATOM | 4696 | OG | SER | I | 173 | 65.691 | −29.253 | −24.924 | 1.00 | 20.73 | O |
| ATOM | 4697 | C | SER | I | 173 | 63.922 | −30.832 | −26.416 | 1.00 | 21.50 | C |
| ATOM | 4698 | O | SER | I | 173 | 64.247 | −32.012 | −26.319 | 1.00 | 21.19 | O |
| ATOM | 4699 | N | GLY | I | 174 | 62.786 | −30.359 | −25.915 | 1.00 | 20.49 | N |
| ATOM | 4700 | CA | GLY | I | 174 | 61.847 | −31.222 | −25.215 | 1.00 | 19.97 | C |
| ATOM | 4701 | C | GLY | I | 174 | 62.108 | −31.235 | −23.718 | 1.00 | 19.46 | C |
| ATOM | 4702 | O | GLY | I | 174 | 61.375 | −31.877 | −22.961 | 1.00 | 19.39 | O |
| ATOM | 4703 | N | LEU | I | 175 | 63.149 | −30.528 | −23.292 | 1.00 | 18.85 | N |
| ATOM | 4704 | CA | LEU | I | 175 | 63.471 | −30.434 | −21.872 | 1.00 | 19.55 | C |
| ATOM | 4705 | CB | LEU | I | 175 | 64.986 | −30.540 | −21.641 | 1.00 | 19.61 | C |
| ATOM | 4706 | CG | LEU | I | 175 | 65.647 | −31.812 | −22.200 | 1.00 | 20.33 | C |
| ATOM | 4707 | CD1 | LEU | I | 175 | 67.182 | −31.739 | −22.139 | 1.00 | 21.36 | C |
| ATOM | 4708 | CD2 | LEU | I | 175 | 65.085 | −33.083 | −21.504 | 1.00 | 19.68 | C |
| ATOM | 4709 | C | LEU | I | 175 | 62.947 | −29.128 | −21.295 | 1.00 | 19.75 | C |
| ATOM | 4710 | O | LEU | I | 175 | 62.853 | −28.114 | −21.992 | 1.00 | 20.05 | O |
| ATOM | 4711 | N | TYR | I | 176 | 62.651 | −29.146 | −20.007 | 1.00 | 20.11 | N |
| ATOM | 4712 | CA | TYR | I | 176 | 62.169 | −27.946 | −19.337 | 1.00 | 20.47 | C |
| ATOM | 4713 | CB | TYR | I | 176 | 61.162 | −28.316 | −18.243 | 1.00 | 20.44 | C |
| ATOM | 4714 | CG | TYR | I | 176 | 59.868 | −28.917 | −18.746 | 1.00 | 22.12 | C |
| ATOM | 4715 | CD1 | TYR | I | 176 | 58.766 | −28.103 | −19.053 | 1.00 | 22.85 | C |
| ATOM | 4716 | CE1 | TYR | I | 176 | 57.562 | −28.657 | −19.504 | 1.00 | 22.60 | C |
| ATOM | 4717 | CZ | TYR | I | 176 | 57.467 | −30.033 | −19.664 | 1.00 | 23.17 | C |
| ATOM | 4718 | OH | TYR | I | 176 | 56.294 | −30.586 | −20.100 | 1.00 | 23.17 | O |
| ATOM | 4719 | CE2 | TYR | I | 176 | 58.543 | −30.866 | −19.365 | 1.00 | 22.30 | C |
| ATOM | 4720 | CD2 | TYR | I | 176 | 59.733 | −30.302 | −18.903 | 1.00 | 22.67 | C |
| ATOM | 4721 | C | TYR | I | 176 | 63.314 | −27.104 | −18.753 | 1.00 | 20.31 | C |
| ATOM | 4722 | O | TYR | I | 176 | 64.413 | −27.603 | −18.477 | 1.00 | 19.72 | O |
| ATOM | 4723 | N | SER | I | 177 | 63.017 | −25.827 | −18.540 | 1.00 | 20.37 | N |
| ATOM | 4724 | CA | SER | I | 177 | 63.928 | −24.884 | −17.919 | 1.00 | 20.93 | C |
| ATOM | 4725 | CB | SER | I | 177 | 64.718 | −24.154 | −19.002 | 1.00 | 20.66 | C |
| ATOM | 4726 | OG | SER | I | 177 | 65.611 | −23.227 | −18.430 | 1.00 | 23.74 | O |
| ATOM | 4727 | C | SER | I | 177 | 63.127 | −23.872 | −17.103 | 1.00 | 21.22 | C |
| ATOM | 4728 | O | SER | I | 177 | 62.074 | −23.387 | −17.541 | 1.00 | 20.46 | O |
| ATOM | 4729 | N | LEU | I | 178 | 63.643 | −23.531 | −15.930 | 1.00 | 21.88 | N |
| ATOM | 4730 | CA | LEU | I | 178 | 62.994 | −22.529 | −15.104 | 1.00 | 22.35 | C |
| ATOM | 4731 | CB | LEU | I | 178 | 61.887 | −23.144 | −14.222 | 1.00 | 22.76 | C |
| ATOM | 4732 | CG | LEU | I | 178 | 62.062 | −23.814 | −12.851 | 1.00 | 23.27 | C |
| ATOM | 4733 | CD1 | LEU | I | 178 | 62.701 | −22.917 | −11.795 | 1.00 | 22.05 | C |
| ATOM | 4734 | CD2 | LEU | I | 178 | 60.704 | −24.256 | −12.348 | 1.00 | 23.88 | C |
| ATOM | 4735 | C | LEU | I | 178 | 63.991 | −21.748 | −14.280 | 1.00 | 22.49 | C |
| ATOM | 4736 | O | LEU | I | 178 | 65.102 | −22.222 | −14.003 | 1.00 | 22.88 | O |
| ATOM | 4737 | N | SER | I | 179 | 63.589 | −20.538 | −13.915 | 1.00 | 21.78 | N |
| ATOM | 4738 | CA | SER | I | 179 | 64.401 | −19.675 | −13.085 | 1.00 | 21.51 | C |
| ATOM | 4739 | CB | SER | I | 179 | 64.743 | −18.404 | −13.838 | 1.00 | 21.79 | C |
| ATOM | 4740 | OG | SER | I | 179 | 65.873 | −18.627 | −14.669 | 1.00 | 26.30 | O |
| ATOM | 4741 | C | SER | I | 179 | 63.669 | −19.334 | −11.791 | 1.00 | 20.34 | C |
| ATOM | 4742 | O | SER | I | 179 | 62.453 | −19.162 | −11.793 | 1.00 | 19.39 | O |
| ATOM | 4743 | N | SER | I | 180 | 64.435 | −19.266 | −10.702 | 1.00 | 19.27 | N |
| ATOM | 4744 | CA | SER | I | 180 | 63.972 | −18.743 | −9.416 | 1.00 | 18.86 | C |
| ATOM | 4745 | CB | SER | I | 180 | 63.998 | −19.821 | −8.332 | 1.00 | 18.90 | C |
| ATOM | 4746 | OG | SER | I | 180 | 63.594 | −19.306 | −7.073 | 1.00 | 20.55 | O |
| ATOM | 4747 | C | SER | I | 180 | 64.900 | −17.609 | −9.044 | 1.00 | 18.19 | C |
| ATOM | 4748 | O | SER | I | 180 | 66.131 | −17.736 | −9.167 | 1.00 | 18.34 | O |
| ATOM | 4749 | N | VAL | I | 181 | 64.315 | −16.491 | −8.622 | 1.00 | 17.22 | N |
| ATOM | 4750 | CA | VAL | I | 181 | 65.089 | −15.319 | −8.216 | 1.00 | 17.18 | C |
| ATOM | 4751 | CB | VAL | I | 181 | 64.995 | −14.166 | −9.236 | 1.00 | 17.22 | C |
| ATOM | 4752 | CG1 | VAL | I | 181 | 65.463 | −14.622 | −10.611 | 1.00 | 16.15 | C |
| ATOM | 4753 | CG2 | VAL | I | 181 | 63.570 | −13.610 | −9.303 | 1.00 | 17.76 | C |
| ATOM | 4754 | C | VAL | I | 181 | 64.651 | −14.818 | −6.833 | 1.00 | 17.39 | C |
| ATOM | 4755 | O | VAL | I | 181 | 63.541 | −15.136 | −6.349 | 1.00 | 16.13 | O |
| ATOM | 4756 | N | VAL | I | 182 | 65.536 | −14.056 | −6.202 | 1.00 | 17.46 | N |
| ATOM | 4757 | CA | VAL | I | 182 | 65.203 | −13.362 | −4.967 | 1.00 | 18.09 | C |
| ATOM | 4758 | CB | VAL | I | 182 | 65.475 | −14.244 | −3.684 | 1.00 | 18.21 | C |
| ATOM | 4759 | CG1 | VAL | I | 182 | 66.908 | −14.761 | −3.647 | 1.00 | 18.13 | C |
| ATOM | 4760 | CG2 | VAL | I | 182 | 65.103 | −13.497 | −2.387 | 1.00 | 17.88 | C |
| ATOM | 4761 | C | VAL | I | 182 | 65.922 | −12.027 | −4.945 | 1.00 | 18.67 | C |
| ATOM | 4762 | O | VAL | I | 182 | 67.036 | −11.892 | −5.457 | 1.00 | 18.90 | O |
| ATOM | 4763 | N | THR | I | 183 | 65.254 | −11.019 | −4.408 | 1.00 | 19.09 | N |
| ATOM | 4764 | CA | THR | I | 183 | 65.876 | −9.735 | −4.228 | 1.00 | 19.67 | C |
| ATOM | 4765 | CB | THR | I | 183 | 64.961 | −8.592 | −4.647 | 1.00 | 19.77 | C |
| ATOM | 4766 | OG1 | THR | I | 183 | 63.692 | −8.751 | −4.014 | 1.00 | 20.46 | O |
| ATOM | 4767 | CG2 | THR | I | 183 | 64.752 | −8.604 | −6.153 | 1.00 | 19.02 | C |
| ATOM | 4768 | C | THR | I | 183 | 66.264 | −9.627 | −2.764 | 1.00 | 20.31 | C |
| ATOM | 4769 | O | THR | I | 183 | 65.497 | −10.017 | −1.878 | 1.00 | 19.80 | O |
| ATOM | 4770 | N | VAL | I | 184 | 67.474 | −9.139 | −2.520 | 1.00 | 20.36 | N |
| ATOM | 4771 | CA | VAL | I | 184 | 68.044 | −9.128 | −1.172 | 1.00 | 21.80 | C |
| ATOM | 4772 | CB | VAL | I | 184 | 69.009 | −10.334 | −0.941 | 1.00 | 21.07 | C |
| ATOM | 4773 | CG1 | VAL | I | 184 | 68.270 | −11.665 | −1.097 | 1.00 | 22.33 | C |
| ATOM | 4774 | CG2 | VAL | I | 184 | 70.221 | −10.262 | −1.879 | 1.00 | 21.11 | C |
| ATOM | 4775 | C | VAL | I | 184 | 68.777 | −7.801 | −0.918 | 1.00 | 22.48 | C |
| ATOM | 4776 | O | VAL | I | 184 | 69.075 | −7.082 | −1.870 | 1.00 | 22.75 | O |
| ATOM | 4777 | N | PRO | I | 185 | 69.086 | −7.478 | 0.356 | 1.00 | 23.29 | N |
| ATOM | 4778 | CA | PRO | I | 185 | 69.796 | −6.232 | 0.584 | 1.00 | 23.95 | C |
| ATOM | 4779 | CB | PRO | I | 185 | 69.823 | −6.113 | 2.114 | 1.00 | 24.01 | C |
| ATOM | 4780 | CG | PRO | I | 185 | 68.776 | −7.056 | 2.602 | 1.00 | 23.61 | C |
| ATOM | 4781 | CD | PRO | I | 185 | 68.822 | −8.179 | 1.623 | 1.00 | 23.37 | C |
| ATOM | 4782 | C | PRO | I | 185 | 71.217 | −6.312 | 0.037 | 1.00 | 24.65 | C |
| ATOM | 4783 | O | PRO | I | 185 | 71.854 | −7.369 | 0.124 | 1.00 | 24.70 | O |
| ATOM | 4784 | N | SER | I | 186 | 71.687 | −5.214 | −0.552 | 1.00 | 25.19 | N |
| ATOM | 4785 | CA | SER | I | 186 | 73.072 | −5.100 | −1.028 | 1.00 | 26.42 | C |
| ATOM | 4786 | CB | SER | I | 186 | 73.290 | −3.761 | −1.742 | 1.00 | 26.23 | C |
| ATOM | 4787 | OG | SER | I | 186 | 72.404 | −3.630 | −2.838 | 1.00 | 26.98 | O |
| ATOM | 4788 | C | SER | I | 186 | 74.060 | −5.198 | 0.123 | 1.00 | 27.17 | C |
| ATOM | 4789 | O | SER | I | 186 | 75.168 | −5.709 | −0.042 | 1.00 | 27.73 | O |
| ATOM | 4790 | N | SER | I | 187 | 73.662 | −4.697 | 1.288 | 1.00 | 27.71 | N |
| ATOM | 4791 | CA | SER | I | 187 | 74.534 | −4.716 | 2.455 | 1.00 | 28.87 | C |
| ATOM | 4792 | CB | SER | I | 187 | 73.944 | −3.863 | 3.583 | 1.00 | 28.69 | C |
| ATOM | 4793 | OG | SER | I | 187 | 72.629 | −4.282 | 3.902 | 1.00 | 29.26 | O |
| ATOM | 4794 | C | SER | I | 187 | 74.837 | −6.138 | 2.944 | 1.00 | 29.28 | C |
| ATOM | 4795 | O | SER | I | 187 | 75.822 | −6.347 | 3.654 | 1.00 | 29.53 | O |
| ATOM | 4796 | N | SER | I | 188 | 74.004 | −7.100 | 2.536 | 1.00 | 29.78 | N |
| ATOM | 4797 | CA | SER | I | 188 | 74.090 | −8.495 | 3.002 | 1.00 | 29.95 | C |
| ATOM | 4798 | CB | SER | I | 188 | 72.681 | −9.088 | 3.145 | 1.00 | 29.89 | C |
| ATOM | 4799 | OG | SER | I | 188 | 72.178 | −9.570 | 1.894 | 1.00 | 30.25 | O |
| ATOM | 4800 | C | SER | I | 188 | 74.940 | −9.418 | 2.113 | 1.00 | 29.99 | C |
| ATOM | 4801 | O | SER | I | 188 | 75.208 | −10.565 | 2.479 | 1.00 | 30.21 | O |
| ATOM | 4802 | N | LEU | I | 189 | 75.351 | −8.933 | 0.947 | 1.00 | 29.94 | N |
| ATOM | 4803 | CA | LEU | I | 189 | 75.981 | −9.809 | −0.045 | 1.00 | 29.91 | C |
| ATOM | 4804 | CB | LEU | I | 189 | 76.060 | −9.119 | −1.410 | 1.00 | 29.94 | C |
| ATOM | 4805 | CG | LEU | I | 189 | 74.747 | −8.922 | −2.172 | 1.00 | 29.33 | C |
| ATOM | 4806 | CD1 | LEU | I | 189 | 75.002 | −8.163 | −3.462 | 1.00 | 29.45 | C |
| ATOM | 4807 | CD2 | LEU | I | 189 | 74.072 | −10.253 | −2.471 | 1.00 | 29.44 | C |
| ATOM | 4808 | C | LEU | I | 189 | 77.348 | −10.368 | 0.373 | 1.00 | 30.02 | C |
| ATOM | 4809 | O | LEU | I | 189 | 77.734 | −11.454 | −0.055 | 1.00 | 29.83 | O |
| ATOM | 4810 | N | GLY | I | 190 | 78.066 | −9.633 | 1.216 | 1.00 | 30.18 | N |
| ATOM | 4811 | CA | GLY | I | 190 | 79.379 | −10.066 | 1.676 | 1.00 | 30.26 | C |
| ATOM | 4812 | C | GLY | I | 190 | 79.378 | −11.014 | 2.862 | 1.00 | 30.40 | C |
| ATOM | 4813 | O | GLY | I | 190 | 80.410 | −11.625 | 3.165 | 1.00 | 30.91 | O |
| ATOM | 4814 | N | THR | I | 191 | 78.241 | −11.137 | 3.544 | 1.00 | 30.01 | N |
| ATOM | 4815 | CA | THR | I | 191 | 78.165 | −11.941 | 4.771 | 1.00 | 29.92 | C |
| ATOM | 4816 | CB | THR | I | 191 | 77.846 | −11.074 | 6.016 | 1.00 | 29.97 | C |
| ATOM | 4817 | OG1 | THR | I | 191 | 76.805 | −10.142 | 5.697 | 1.00 | 30.34 | O |
| ATOM | 4818 | CG2 | THR | I | 191 | 79.088 | −10.310 | 6.483 | 1.00 | 30.54 | C |
| ATOM | 4819 | C | THR | I | 191 | 77.156 | −13.090 | 4.673 | 1.00 | 29.71 | C |
| ATOM | 4820 | O | THR | I | 191 | 77.541 | −14.266 | 4.756 | 1.00 | 30.27 | O |
| ATOM | 4821 | N | GLN | I | 192 | 75.878 | −12.753 | 4.489 | 1.00 | 28.58 | N |
| ATOM | 4822 | CA | GLN | I | 192 | 74.818 | −13.751 | 4.380 | 1.00 | 27.66 | C |
| ATOM | 4823 | CB | GLN | I | 192 | 73.435 | −13.076 | 4.406 | 1.00 | 27.73 | C |
| ATOM | 4824 | CG | GLN | I | 192 | 72.236 | −14.004 | 4.094 | 1.00 | 29.05 | C |
| ATOM | 4825 | CD | GLN | I | 192 | 71.830 | −14.901 | 5.257 | 1.00 | 30.16 | C |
| ATOM | 4826 | OE1 | GLN | I | 192 | 71.686 | −14.442 | 6.392 | 1.00 | 30.00 | O |
| ATOM | 4827 | NE2 | GLN | I | 192 | 71.618 | −16.186 | 4.970 | 1.00 | 29.14 | N |
| ATOM | 4828 | C | GLN | I | 192 | 74.995 | −14.626 | 3.131 | 1.00 | 26.52 | C |
| ATOM | 4829 | O | GLN | I | 192 | 75.203 | −14.116 | 2.024 | 1.00 | 26.60 | O |
| ATOM | 4830 | N | THR | I | 193 | 74.925 | −15.940 | 3.319 | 1.00 | 24.70 | N |
| ATOM | 4831 | CA | THR | I | 193 | 75.042 | −16.873 | 2.201 | 1.00 | 23.06 | C |
| ATOM | 4832 | CB | THR | I | 193 | 75.770 | −18.199 | 2.602 | 1.00 | 23.40 | C |
| ATOM | 4833 | OG1 | THR | I | 193 | 74.829 | −19.159 | 3.088 | 1.00 | 23.61 | O |
| ATOM | 4834 | CG2 | THR | I | 193 | 76.845 | −17.959 | 3.669 | 1.00 | 22.37 | C |
| ATOM | 4835 | C | THR | I | 193 | 73.659 | −17.157 | 1.594 | 1.00 | 22.03 | C |
| ATOM | 4836 | O | THR | I | 193 | 72.655 | −17.203 | 2.312 | 1.00 | 20.71 | O |
| ATOM | 4837 | N | TYR | I | 194 | 73.606 | −17.314 | 0.273 | 1.00 | 20.70 | N |
| ATOM | 4838 | CA | TYR | I | 194 | 72.362 | −17.707 | −0.394 | 1.00 | 20.20 | C |
| ATOM | 4839 | CB | TYR | I | 194 | 71.815 | −16.588 | −1.280 | 1.00 | 19.91 | C |
| ATOM | 4840 | CG | TYR | I | 194 | 71.544 | −15.325 | −0.513 | 1.00 | 20.04 | C |
| ATOM | 4841 | CD1 | TYR | I | 194 | 72.462 | −14.281 | −0.505 | 1.00 | 19.23 | C |
| ATOM | 4842 | CE1 | TYR | I | 194 | 72.210 | −13.116 | 0.206 | 1.00 | 19.74 | C |
| ATOM | 4843 | CZ | TYR | I | 194 | 71.033 | −12.992 | 0.924 | 1.00 | 19.59 | C |
| ATOM | 4844 | OH | TYR | I | 194 | 70.768 | −11.843 | 1.637 | 1.00 | 22.15 | O |
| ATOM | 4845 | CE2 | TYR | I | 194 | 70.115 | −14.019 | 0.943 | 1.00 | 19.56 | C |
| ATOM | 4846 | CD2 | TYR | I | 194 | 70.365 | −15.176 | 0.223 | 1.00 | 19.52 | C |
| ATOM | 4847 | C | TYR | I | 194 | 72.545 | −18.980 | −1.186 | 1.00 | 19.86 | C |
| ATOM | 4848 | O | TYR | I | 194 | 73.433 | −19.084 | −2.038 | 1.00 | 19.86 | O |
| ATOM | 4849 | N | ILE | I | 195 | 71.699 | −19.957 | −0.886 | 1.00 | 19.56 | N |
| ATOM | 4850 | CA | ILE | I | 195 | 71.770 | −21.250 | −1.534 | 1.00 | 19.44 | C |
| ATOM | 4851 | CB | ILE | I | 195 | 72.255 | −22.335 | −0.539 | 1.00 | 19.27 | C |
| ATOM | 4852 | CG1 | ILE | I | 195 | 73.660 | −21.990 | −0.019 | 1.00 | 19.35 | C |
| ATOM | 4853 | CD1 | ILE | I | 195 | 73.952 | −22.442 | 1.392 | 1.00 | 20.88 | C |
| ATOM | 4854 | CG2 | ILE | I | 195 | 72.231 | −23.713 | −1.193 | 1.00 | 19.89 | C |
| ATOM | 4855 | C | ILE | I | 195 | 70.389 | −21.600 | −2.047 | 1.00 | 19.52 | C |
| ATOM | 4856 | O | ILE | I | 195 | 69.419 | −21.587 | −1.280 | 1.00 | 19.30 | O |
| ATOM | 4857 | N | CYS | I | 196 | 70.278 | −21.898 | −3.334 | 1.00 | 19.69 | N |
| ATOM | 4858 | CA | CYS | I | 196 | 69.025 | −22.473 | −3.817 | 1.00 | 20.39 | C |
| ATOM | 4859 | CB | CYS | I | 196 | 68.696 | −22.059 | −5.249 | 1.00 | 20.75 | C |
| ATOM | 4860 | SG | CYS | I | 196 | 69.779 | −22.752 | −6.479 | 1.00 | 25.21 | S |
| ATOM | 4861 | C | CYS | I | 196 | 69.047 | −23.988 | −3.686 | 1.00 | 20.13 | C |
| ATOM | 4862 | O | CYS | I | 196 | 70.002 | −24.636 | −4.099 | 1.00 | 19.82 | O |
| ATOM | 4863 | N | ASN | I | 197 | 67.983 | −24.545 | −3.109 | 1.00 | 20.03 | N |
| ATOM | 4864 | CA | ASN | I | 197 | 67.863 | −25.987 | −2.964 | 1.00 | 20.22 | C |
| ATOM | 4865 | CB | ASN | I | 197 | 67.400 | −26.348 | −1.557 | 1.00 | 20.48 | C |
| ATOM | 4866 | CG | ASN | I | 197 | 67.840 | −25.335 | −0.535 | 1.00 | 20.71 | C |
| ATOM | 4867 | OD1 | ASN | I | 197 | 67.058 | −24.466 | −0.124 | 1.00 | 21.10 | O |
| ATOM | 4868 | ND2 | ASN | I | 197 | 69.106 | −25.409 | −0.140 | 1.00 | 20.33 | N |
| ATOM | 4869 | C | ASN | I | 197 | 66.867 | −26.483 | −3.989 | 1.00 | 20.15 | C |
| ATOM | 4870 | O | ASN | I | 197 | 65.690 | −26.131 | −3.934 | 1.00 | 19.48 | O |
| ATOM | 4871 | N | VAL | I | 198 | 67.355 | −27.279 | −4.935 | 1.00 | 20.39 | N |
| ATOM | 4872 | CA | VAL | I | 198 | 66.557 | −27.733 | −6.066 | 1.00 | 21.29 | C |
| ATOM | 4873 | CB | VAL | I | 198 | 67.290 | −27.451 | −7.403 | 1.00 | 21.01 | C |
| ATOM | 4874 | CG1 | VAL | I | 198 | 66.496 | −27.965 | −8.592 | 1.00 | 21.13 | C |
| ATOM | 4875 | CG2 | VAL | I | 198 | 67.550 | −25.947 | −7.559 | 1.00 | 21.50 | C |
| ATOM | 4876 | C | VAL | I | 198 | 66.295 | −29.226 | −5.895 | 1.00 | 22.21 | C |
| ATOM | 4877 | O | VAL | I | 198 | 67.220 | −29.983 | −5.619 | 1.00 | 22.29 | O |
| ATOM | 4878 | N | ASN | I | 199 | 65.033 | −29.630 | −6.041 | 1.00 | 23.25 | N |
| ATOM | 4879 | CA | ASN | I | 199 | 64.634 | −31.030 | −5.925 | 1.00 | 24.36 | C |
| ATOM | 4880 | CB | ASN | I | 199 | 63.810 | −31.239 | −4.641 | 1.00 | 24.71 | C |
| ATOM | 4881 | CG | ASN | I | 199 | 63.654 | −32.716 | −4.252 | 1.00 | 27.72 | C |
| ATOM | 4882 | OD1 | ASN | I | 199 | 64.311 | −33.611 | −4.807 | 1.00 | 30.66 | O |
| ATOM | 4883 | ND2 | ASN | I | 199 | 62.785 | −32.971 | −3.275 | 1.00 | 30.18 | N |
| ATOM | 4884 | C | ASN | I | 199 | 63.836 | −31.458 | −7.160 | 1.00 | 24.55 | C |
| ATOM | 4885 | O | ASN | I | 199 | 62.826 | −30.832 | −7.501 | 1.00 | 24.52 | O |
| ATOM | 4886 | N | HIS | I | 200 | 64.320 | −32.503 | −7.835 | 1.00 | 24.81 | N |
| ATOM | 4887 | CA | HIS | I | 200 | 63.637 | −33.106 | −8.978 | 1.00 | 25.13 | C |
| ATOM | 4888 | CB | HIS | I | 200 | 64.468 | −32.953 | −10.252 | 1.00 | 24.70 | C |
| ATOM | 4889 | CG | HIS | I | 200 | 63.768 | −33.409 | −11.495 | 1.00 | 24.37 | C |
| ATOM | 4890 | ND1 | HIS | I | 200 | 64.223 | −34.461 | −12.255 | 1.00 | 24.23 | N |
| ATOM | 4891 | CE1 | HIS | I | 200 | 63.415 | −34.644 | −13.285 | 1.00 | 22.90 | C |
| ATOM | 4892 | NE2 | HIS | I | 200 | 62.452 | −33.745 | −13.221 | 1.00 | 22.36 | N |
| ATOM | 4893 | CD2 | HIS | I | 200 | 62.646 | −32.961 | −12.108 | 1.00 | 22.67 | C |
| ATOM | 4894 | C | HIS | I | 200 | 63.359 | −34.576 | −8.680 | 1.00 | 25.74 | C |
| ATOM | 4895 | O | HIS | I | 200 | 64.180 | −35.454 | −8.958 | 1.00 | 25.35 | O |
| ATOM | 4896 | N | LYS | I | 201 | 62.185 | −34.814 | −8.104 | 1.00 | 26.85 | N |
| ATOM | 4897 | CA | LYS | I | 201 | 61.772 | −36.138 | −7.647 | 1.00 | 28.03 | C |
| ATOM | 4898 | CB | LYS | I | 201 | 60.403 | −36.063 | −6.950 | 1.00 | 28.51 | C |
| ATOM | 4899 | CG | LYS | I | 201 | 60.025 | −37.325 | −6.147 | 1.00 | 30.35 | C |
| ATOM | 4900 | CD | LYS | I | 201 | 60.706 | −37.352 | −4.779 | 1.00 | 33.85 | C |
| ATOM | 4901 | CE | LYS | I | 201 | 60.360 | −38.616 | −3.990 | 1.00 | 35.55 | C |
| ATOM | 4902 | NZ | LYS | I | 201 | 61.286 | −38.772 | −2.826 | 1.00 | 36.81 | N |
| ATOM | 4903 | C | LYS | I | 201 | 61.797 | −37.223 | −8.743 | 1.00 | 28.10 | C |
| ATOM | 4904 | O | LYS | I | 201 | 62.249 | −38.329 | −8.479 | 1.00 | 28.00 | O |
| ATOM | 4905 | N | PRO | I | 202 | 61.347 | −36.908 | −9.978 | 1.00 | 28.30 | N |
| ATOM | 4906 | CA | PRO | I | 202 | 61.395 | −37.939 | −11.019 | 1.00 | 28.69 | C |
| ATOM | 4907 | CB | PRO | I | 202 | 60.915 | −37.189 | −12.261 | 1.00 | 28.51 | C |
| ATOM | 4908 | CG | PRO | I | 202 | 59.931 | −36.225 | −11.714 | 1.00 | 28.34 | C |
| ATOM | 4909 | CD | PRO | I | 202 | 60.572 | −35.737 | −10.429 | 1.00 | 28.47 | C |
| ATOM | 4910 | C | PRO | I | 202 | 62.767 | −38.575 | −11.268 | 1.00 | 29.08 | C |
| ATOM | 4911 | O | PRO | I | 202 | 62.824 | −39.735 | −11.682 | 1.00 | 29.63 | O |
| ATOM | 4912 | N | SER | I | 203 | 63.851 | −37.836 | −11.016 | 1.00 | 29.12 | N |
| ATOM | 4913 | CA | SER | I | 203 | 65.206 | −38.340 | −11.235 | 1.00 | 29.41 | C |
| ATOM | 4914 | CB | SER | I | 203 | 65.985 | −37.403 | −12.168 | 1.00 | 29.35 | C |
| ATOM | 4915 | OG | SER | I | 203 | 66.269 | −36.161 | −11.534 | 1.00 | 27.78 | O |
| ATOM | 4916 | C | SER | I | 203 | 65.992 | −38.549 | −9.935 | 1.00 | 30.07 | C |
| ATOM | 4917 | O | SER | I | 203 | 67.172 | −38.899 | −9.972 | 1.00 | 29.87 | O |
| ATOM | 4918 | N | ASN | I | 204 | 65.329 | −38.328 | −8.798 | 1.00 | 31.02 | N |
| ATOM | 4919 | CA | ASN | I | 204 | 65.964 | −38.370 | −7.466 | 1.00 | 31.97 | C |
| ATOM | 4920 | CB | ASN | I | 204 | 66.254 | −39.815 | −7.025 | 1.00 | 32.36 | C |
| ATOM | 4921 | CG | ASN | I | 204 | 64.995 | −40.663 | −6.934 | 1.00 | 34.06 | C |
| ATOM | 4922 | OD1 | ASN | I | 204 | 63.910 | −40.171 | −6.594 | 1.00 | 35.48 | O |
| ATOM | 4923 | ND2 | ASN | I | 204 | 65.132 | −41.949 | −7.244 | 1.00 | 35.47 | N |
| ATOM | 4924 | C | ASN | I | 204 | 67.215 | −37.495 | −7.382 | 1.00 | 32.14 | C |
| ATOM | 4925 | O | ASN | I | 204 | 68.254 | −37.913 | −6.855 | 1.00 | 32.06 | O |
| ATOM | 4926 | N | THR | I | 205 | 67.090 | −36.278 | −7.918 | 1.00 | 32.04 | N |
| ATOM | 4927 | CA | THR | I | 205 | 68.165 | −35.298 | −7.944 | 1.00 | 32.17 | C |
| ATOM | 4928 | CB | THR | I | 205 | 68.309 | −34.649 | −9.346 | 1.00 | 32.28 | C |
| ATOM | 4929 | OG1 | THR | I | 205 | 68.572 | −35.660 | −10.322 | 1.00 | 32.22 | O |
| ATOM | 4930 | CG2 | THR | I | 205 | 69.451 | −33.637 | −9.373 | 1.00 | 32.50 | C |
| ATOM | 4931 | C | THR | I | 205 | 67.899 | −34.202 | −6.918 | 1.00 | 32.16 | C |
| ATOM | 4932 | O | THR | I | 205 | 66.830 | −33.583 | −6.912 | 1.00 | 32.20 | O |
| ATOM | 4933 | N | LYS | I | 206 | 68.870 | −33.979 | −6.044 | 1.00 | 31.77 | N |
| ATOM | 4934 | CA | LYS | I | 206 | 68.828 | −32.846 | −5.148 | 1.00 | 31.78 | C |
| ATOM | 4935 | CB | LYS | I | 206 | 68.643 | −33.285 | −3.695 | 1.00 | 32.07 | C |
| ATOM | 4936 | CG | LYS | I | 206 | 67.236 | −33.695 | −3.326 | 1.00 | 33.47 | C |
| ATOM | 4937 | CD | LYS | I | 206 | 67.150 | −33.976 | −1.832 | 1.00 | 36.48 | C |
| ATOM | 4938 | CE | LYS | I | 206 | 65.800 | −34.545 | −1.434 | 1.00 | 37.34 | C |
| ATOM | 4939 | NZ | LYS | I | 206 | 65.837 | −35.068 | −0.033 | 1.00 | 37.69 | N |
| ATOM | 4940 | C | LYS | I | 206 | 70.128 | −32.079 | −5.299 | 1.00 | 31.44 | C |
| ATOM | 4941 | O | LYS | I | 206 | 71.220 | −32.649 | −5.149 | 1.00 | 31.26 | O |
| ATOM | 4942 | N | VAL | I | 207 | 70.001 | −30.789 | −5.607 | 1.00 | 30.42 | N |
| ATOM | 4943 | CA | VAL | I | 207 | 71.153 | −29.906 | −5.771 | 1.00 | 29.68 | C |
| ATOM | 4944 | CB | VAL | I | 207 | 71.272 | −29.364 | −7.220 | 1.00 | 29.62 | C |
| ATOM | 4945 | CG1 | VAL | I | 207 | 72.702 | −28.960 | −7.520 | 1.00 | 29.59 | C |
| ATOM | 4946 | CG2 | VAL | I | 207 | 70.786 | −30.389 | −8.241 | 1.00 | 30.68 | C |
| ATOM | 4947 | C | VAL | I | 207 | 71.000 | −28.719 | −4.829 | 1.00 | 28.95 | C |
| ATOM | 4948 | O | VAL | I | 207 | 69.917 | −28.121 | −4.747 | 1.00 | 28.71 | O |
| ATOM | 4949 | N | ASP | I | 208 | 72.070 | −28.398 | −4.109 | 1.00 | 27.78 | N |
| ATOM | 4950 | CA | ASP | I | 208 | 72.142 | −27.165 | −3.331 | 1.00 | 27.40 | C |
| ATOM | 4951 | CB | ASP | I | 208 | 72.431 | −27.447 | −1.852 | 1.00 | 27.47 | C |
| ATOM | 4952 | CG | ASP | I | 208 | 71.383 | −28.341 | −1.204 | 1.00 | 28.48 | C |
| ATOM | 4953 | OD1 | ASP | I | 208 | 70.178 | −28.153 | −1.475 | 1.00 | 28.41 | O |
| ATOM | 4954 | OD2 | ASP | I | 208 | 71.772 | −29.239 | −0.420 | 1.00 | 30.04 | O |
| ATOM | 4955 | C | ASP | I | 208 | 73.251 | −26.313 | −3.935 | 1.00 | 27.01 | C |
| ATOM | 4956 | O | ASP | I | 208 | 74.416 | −26.692 | −3.896 | 1.00 | 26.32 | O |
| ATOM | 4957 | N | LYS | I | 209 | 72.882 | −25.178 | −4.515 | 1.00 | 26.40 | N |
| ATOM | 4958 | CA | LYS | I | 209 | 73.843 | −24.363 | −5.242 | 1.00 | 26.51 | C |
| ATOM | 4959 | CB | LYS | I | 209 | 73.410 | −24.215 | −6.716 | 1.00 | 26.31 | C |
| ATOM | 4960 | CG | LYS | I | 209 | 74.423 | −23.493 | −7.621 | 1.00 | 27.00 | C |
| ATOM | 4961 | CD | LYS | I | 209 | 75.561 | −24.413 | −8.057 | 1.00 | 28.70 | C |
| ATOM | 4962 | CE | LYS | I | 209 | 76.717 | −23.612 | −8.652 | 1.00 | 31.19 | C |
| ATOM | 4963 | NZ | LYS | I | 209 | 77.993 | −24.401 | −8.722 | 1.00 | 32.29 | N |
| ATOM | 4964 | C | LYS | I | 209 | 74.019 | −23.007 | −4.569 | 1.00 | 26.58 | C |
| ATOM | 4965 | O | LYS | I | 209 | 73.074 | −22.220 | −4.488 | 1.00 | 25.94 | O |
| ATOM | 4966 | N | ARG | I | 210 | 75.220 | −22.746 | −4.060 | 1.00 | 26.92 | N |
| ATOM | 4967 | CA | ARG | I | 210 | 75.515 | −21.448 | −3.462 | 1.00 | 27.77 | C |
| ATOM | 4968 | CB | ARG | I | 210 | 76.772 | −21.496 | −2.581 | 1.00 | 28.24 | C |
| ATOM | 4969 | CG | ARG | I | 210 | 77.063 | −20.172 | −1.846 | 1.00 | 29.78 | C |
| ATOM | 4970 | CD | ARG | I | 210 | 78.456 | −20.148 | −1.248 | 1.00 | 34.48 | C |
| ATOM | 4971 | NE | ARG | I | 210 | 78.540 | −21.011 | −0.070 | 1.00 | 37.31 | N |
| ATOM | 4972 | CZ | ARG | I | 210 | 78.865 | −20.594 | 1.150 | 1.00 | 38.12 | C |
| ATOM | 4973 | NH1 | ARG | I | 210 | 79.168 | −19.320 | 1.368 | 1.00 | 38.16 | N |
| ATOM | 4974 | NH2 | ARG | I | 210 | 78.899 | −21.464 | 2.156 | 1.00 | 39.37 | N |
| ATOM | 4975 | C | ARG | I | 210 | 75.656 | −20.393 | −4.560 | 1.00 | 27.99 | C |
| ATOM | 4976 | O | ARG | I | 210 | 76.348 | −20.611 | −5.564 | 1.00 | 27.75 | O |
| ATOM | 4977 | N | VAL | I | 211 | 74.982 | −19.263 | −4.362 | 1.00 | 28.00 | N |
| ATOM | 4978 | CA | VAL | I | 211 | 75.019 | −18.158 | −5.315 | 1.00 | 28.65 | C |
| ATOM | 4979 | CB | VAL | I | 211 | 73.596 | −17.744 | −5.775 | 1.00 | 28.31 | C |
| ATOM | 4980 | CG1 | VAL | I | 211 | 73.655 | −16.624 | −6.816 | 1.00 | 27.87 | C |
| ATOM | 4981 | CG2 | VAL | I | 211 | 72.840 | −18.937 | −6.317 | 1.00 | 27.88 | C |
| ATOM | 4982 | C | VAL | I | 211 | 75.713 | −16.979 | −4.650 | 1.00 | 29.50 | C |
| ATOM | 4983 | O | VAL | I | 211 | 75.178 | −16.372 | −3.723 | 1.00 | 29.58 | O |
| ATOM | 4984 | N | GLU | I | 212 | 76.915 | −16.675 | −5.118 | 1.00 | 30.75 | N |
| ATOM | 4985 | CA | GLU | I | 212 | 77.707 | −15.582 | −4.559 | 1.00 | 31.97 | C |
| ATOM | 4986 | CB | GLU | I | 212 | 78.948 | −16.130 | −3.849 | 1.00 | 32.26 | C |
| ATOM | 4987 | CG | GLU | I | 212 | 79.795 | −17.073 | −4.679 | 1.00 | 33.61 | C |
| ATOM | 4988 | CD | GLU | I | 212 | 80.460 | −18.131 | −3.825 | 1.00 | 36.37 | C |
| ATOM | 4989 | OE1 | GLU | I | 212 | 81.153 | −17.755 | −2.859 | 1.00 | 36.84 | O |
| ATOM | 4990 | OE2 | GLU | I | 212 | 80.280 | −19.339 | −4.115 | 1.00 | 37.96 | O |
| ATOM | 4991 | C | GLU | I | 212 | 78.106 | −14.572 | −5.631 | 1.00 | 32.43 | C |
| ATOM | 4992 | O | GLU | I | 212 | 78.156 | −14.917 | −6.814 | 1.00 | 32.11 | O |
| ATOM | 4993 | N | PRO | I | 213 | 78.381 | −13.318 | −5.221 | 1.00 | 33.35 | N |
| ATOM | 4994 | CA | PRO | I | 213 | 78.910 | −12.300 | −6.135 | 1.00 | 34.16 | C |
| ATOM | 4995 | CB | PRO | I | 213 | 79.347 | −11.175 | −5.189 | 1.00 | 34.13 | C |
| ATOM | 4996 | CG | PRO | I | 213 | 78.460 | −11.313 | −4.019 | 1.00 | 33.81 | C |
| ATOM | 4997 | CD | PRO | I | 213 | 78.177 | −12.779 | −3.861 | 1.00 | 33.34 | C |
| ATOM | 4998 | C | PRO | I | 213 | 80.120 | −12.810 | −6.915 | 1.00 | 34.85 | C |
| ATOM | 4999 | O | PRO | I | 213 | 80.856 | −13.671 | −6.417 | 1.00 | 34.90 | O |
| ATOM | 5000 | N | LYS | I | 214 | 80.289 | −12.296 | −8.134 | 1.00 | 35.71 | N |
| ATOM | 5001 | CA | LYS | I | 214 | 81.440 | −12.591 | −9.000 | 1.00 | 36.51 | C |
| ATOM | 5002 | CB | LYS | I | 214 | 82.765 | −12.502 | −8.225 | 1.00 | 36.52 | C |
| ATOM | 5003 | CG | LYS | I | 214 | 83.981 | −12.305 | −9.092 | 1.00 | 37.48 | C |
| ATOM | 5004 | CD | LYS | I | 214 | 85.253 | −12.240 | −8.266 | 1.00 | 38.29 | C |
| ATOM | 5005 | CE | LYS | I | 214 | 86.379 | −11.628 | −9.079 | 1.00 | 38.93 | C |
| ATOM | 5006 | NZ | LYS | I | 214 | 86.612 | −12.393 | −10.340 | 1.00 | 39.71 | N |
| ATOM | 5007 | C | LYS | I | 214 | 81.299 | −13.944 | −9.693 | 1.00 | 36.86 | C |
| ATOM | 5008 | O | LYS | I | 214 | 81.578 | −14.066 | −10.889 | 1.00 | 37.39 | O |
| ATOM | 5009 | N | ASP | M | 1 | 27.243 | −28.568 | −31.268 | 1.00 | 41.18 | N |
| ATOM | 5010 | CA | ASP | M | 1 | 28.419 | −28.091 | −30.488 | 1.00 | 41.16 | C |
| ATOM | 5011 | CB | ASP | M | 1 | 29.346 | −27.228 | −31.367 | 1.00 | 41.44 | C |
| ATOM | 5012 | CG | ASP | M | 1 | 30.719 | −26.960 | −30.718 | 1.00 | 42.25 | C |
| ATOM | 5013 | OD1 | ASP | M | 1 | 30.891 | −27.176 | −29.489 | 1.00 | 42.93 | O |
| ATOM | 5014 | OD2 | ASP | M | 1 | 31.639 | −26.525 | −31.449 | 1.00 | 42.50 | O |
| ATOM | 5015 | C | ASP | M | 1 | 27.984 | −27.315 | −29.246 | 1.00 | 40.60 | C |
| ATOM | 5016 | O | ASP | M | 1 | 28.316 | −27.710 | −28.128 | 1.00 | 41.24 | O |
| ATOM | 5017 | N | VAL | M | 2 | 27.231 | −26.231 | −29.452 | 1.00 | 39.64 | N |
| ATOM | 5018 | CA | VAL | M | 2 | 26.907 | −25.241 | −28.397 | 1.00 | 38.08 | C |
| ATOM | 5019 | CB | VAL | M | 2 | 26.018 | −25.813 | −27.225 | 1.00 | 38.30 | C |
| ATOM | 5020 | CG1 | VAL | M | 2 | 25.624 | −24.703 | −26.238 | 1.00 | 37.78 | C |
| ATOM | 5021 | CG2 | VAL | M | 2 | 24.768 | −26.516 | −27.765 | 1.00 | 37.75 | C |
| ATOM | 5022 | C | VAL | M | 2 | 28.177 | −24.541 | −27.868 | 1.00 | 37.25 | C |
| ATOM | 5023 | O | VAL | M | 2 | 28.662 | −24.829 | −26.762 | 1.00 | 37.10 | O |
| ATOM | 5024 | N | VAL | M | 3 | 28.708 | −23.631 | −28.684 | 1.00 | 35.74 | N |
| ATOM | 5025 | CA | VAL | M | 3 | 29.821 | −22.776 | −28.295 | 1.00 | 34.17 | C |
| ATOM | 5026 | CB | VAL | M | 3 | 30.344 | −21.953 | −29.497 | 1.00 | 34.56 | C |
| ATOM | 5027 | CG1 | VAL | M | 3 | 31.684 | −21.289 | −29.162 | 1.00 | 34.29 | C |
| ATOM | 5028 | CG2 | VAL | M | 3 | 30.477 | −22.832 | −30.752 | 1.00 | 34.27 | C |
| ATOM | 5029 | C | VAL | M | 3 | 29.375 | −21.826 | −27.165 | 1.00 | 33.06 | C |
| ATOM | 5030 | O | VAL | M | 3 | 28.272 | −21.270 | −27.208 | 1.00 | 32.68 | O |
| ATOM | 5031 | N | MET | M | 4 | 30.229 | −21.666 | −26.156 | 1.00 | 31.06 | N |
| ATOM | 5032 | CA | MET | M | 4 | 29.964 | −20.759 | −25.043 | 1.00 | 29.97 | C |
| ATOM | 5033 | CB | MET | M | 4 | 30.034 | −21.512 | −23.720 | 1.00 | 29.38 | C |
| ATOM | 5034 | CG | MET | M | 4 | 29.108 | −22.712 | −23.665 | 1.00 | 29.53 | C |
| ATOM | 5035 | SD | MET | M | 4 | 27.486 | −22.273 | −23.038 | 1.00 | 27.43 | S |
| ATOM | 5036 | CE | MET | M | 4 | 27.829 | −22.293 | −21.283 | 1.00 | 27.75 | C |
| ATOM | 5037 | C | MET | M | 4 | 31.008 | −19.663 | −25.065 | 1.00 | 29.04 | C |
| ATOM | 5038 | O | MET | M | 4 | 32.195 | −19.945 | −24.923 | 1.00 | 29.07 | O |
| ATOM | 5039 | N | THR | M | 5 | 30.567 | −18.424 | −25.258 | 1.00 | 28.19 | N |
| ATOM | 5040 | CA | THR | M | 5 | 31.481 | −17.278 | −25.380 | 1.00 | 27.55 | C |
| ATOM | 5041 | CB | THR | M | 5 | 31.198 | −16.466 | −26.679 | 1.00 | 27.42 | C |
| ATOM | 5042 | OG1 | THR | M | 5 | 31.145 | −17.356 | −27.801 | 1.00 | 28.29 | O |
| ATOM | 5043 | CG2 | THR | M | 5 | 32.290 | −15.429 | −26.949 | 1.00 | 27.15 | C |
| ATOM | 5044 | C | THR | M | 5 | 31.373 | −16.388 | −24.127 | 1.00 | 27.24 | C |
| ATOM | 5045 | O | THR | M | 5 | 30.289 | −15.920 | −23.786 | 1.00 | 26.64 | O |
| ATOM | 5046 | N | GLN | M | 6 | 32.502 | −16.191 | −23.446 | 1.00 | 27.09 | N |
| ATOM | 5047 | CA | GLN | M | 6 | 32.581 | −15.371 | −22.245 | 1.00 | 27.05 | C |
| ATOM | 5048 | CB | GLN | M | 6 | 33.328 | −16.105 | −21.130 | 1.00 | 27.25 | C |
| ATOM | 5049 | CG | GLN | M | 6 | 32.453 | −17.008 | −20.290 | 1.00 | 25.87 | C |
| ATOM | 5050 | CD | GLN | M | 6 | 33.206 | −17.697 | −19.164 | 1.00 | 24.39 | C |
| ATOM | 5051 | OE1 | GLN | M | 6 | 33.432 | −18.908 | −19.215 | 1.00 | 23.00 | O |
| ATOM | 5052 | NE2 | GLN | M | 6 | 33.588 | −16.935 | −18.140 | 1.00 | 22.12 | N |
| ATOM | 5053 | C | GLN | M | 6 | 33.286 | −14.056 | −22.519 | 1.00 | 27.49 | C |
| ATOM | 5054 | O | GLN | M | 6 | 34.288 | −14.020 | −23.223 | 1.00 | 27.05 | O |
| ATOM | 5055 | N | THR | M | 7 | 32.753 | −12.983 | −21.946 | 1.00 | 28.13 | N |
| ATOM | 5056 | CA | THR | M | 7 | 33.357 | −11.655 | −22.038 | 1.00 | 28.88 | C |
| ATOM | 5057 | CB | THR | M | 7 | 32.673 | −10.775 | −23.115 | 1.00 | 29.13 | C |
| ATOM | 5058 | OG1 | THR | M | 7 | 31.313 | −10.548 | −22.742 | 1.00 | 29.68 | O |
| ATOM | 5059 | CG2 | THR | M | 7 | 32.727 | −11.418 | −24.503 | 1.00 | 29.39 | C |
| ATOM | 5060 | C | THR | M | 7 | 33.258 | −10.923 | −20.685 | 1.00 | 28.92 | C |
| ATOM | 5061 | O | THR | M | 7 | 32.258 | −11.082 | −19.964 | 1.00 | 29.32 | O |
| ATOM | 5062 | N | PRO | M | 8 | 34.302 | −10.148 | −20.318 | 1.00 | 28.71 | N |
| ATOM | 5063 | CA | PRO | M | 8 | 35.589 | −10.039 | −21.007 | 1.00 | 28.50 | C |
| ATOM | 5064 | CB | PRO | M | 8 | 36.191 | −8.769 | −20.398 | 1.00 | 28.87 | C |
| ATOM | 5065 | CG | PRO | M | 8 | 35.657 | −8.776 | −18.975 | 1.00 | 28.08 | C |
| ATOM | 5066 | CD | PRO | M | 8 | 34.251 | −9.297 | −19.108 | 1.00 | 28.57 | C |
| ATOM | 5067 | C | PRO | M | 8 | 36.456 | −11.239 | −20.649 | 1.00 | 28.76 | C |
| ATOM | 5068 | O | PRO | M | 8 | 36.065 | −12.045 | −19.783 | 1.00 | 28.82 | O |
| ATOM | 5069 | N | LEU | M | 9 | 37.623 | −11.354 | −21.279 | 1.00 | 28.58 | N |
| ATOM | 5070 | CA | LEU | M | 9 | 38.553 | −12.448 | −20.981 | 1.00 | 28.88 | C |
| ATOM | 5071 | CB | LEU | M | 9 | 39.475 | −12.734 | −22.178 | 1.00 | 29.59 | C |
| ATOM | 5072 | CG | LEU | M | 9 | 38.833 | −13.464 | −23.363 | 1.00 | 30.11 | C |
| ATOM | 5073 | CD1 | LEU | M | 9 | 39.798 | −13.565 | −24.560 | 1.00 | 32.97 | C |
| ATOM | 5074 | CD2 | LEU | M | 9 | 38.339 | −14.864 | −22.941 | 1.00 | 32.43 | C |
| ATOM | 5075 | C | LEU | M | 9 | 39.384 | −12.211 | −19.720 | 1.00 | 28.75 | C |
| ATOM | 5076 | O | LEU | M | 9 | 39.730 | −13.164 | −19.024 | 1.00 | 28.80 | O |
| ATOM | 5077 | N | SER | M | 10 | 39.728 | −10.949 | −19.462 | 1.00 | 28.27 | N |
| ATOM | 5078 | CA | SER | M | 10 | 40.465 | −10.546 | −18.265 | 1.00 | 28.26 | C |
| ATOM | 5079 | CB | SER | M | 10 | 41.844 | −9.964 | −18.598 | 1.00 | 28.71 | C |
| ATOM | 5080 | OG | SER | M | 10 | 42.205 | −10.210 | −19.940 | 1.00 | 32.28 | O |
| ATOM | 5081 | C | SER | M | 10 | 39.668 | −9.438 | −17.620 | 1.00 | 27.22 | C |
| ATOM | 5082 | O | SER | M | 10 | 39.090 | −8.577 | −18.314 | 1.00 | 26.33 | O |
| ATOM | 5083 | N | LEU | M | 11 | 39.653 | −9.443 | −16.294 | 1.00 | 25.92 | N |
| ATOM | 5084 | CA | LEU | M | 11 | 38.930 | −8.427 | −15.557 | 1.00 | 25.12 | C |
| ATOM | 5085 | CB | LEU | M | 11 | 37.530 | −8.935 | −15.200 | 1.00 | 25.26 | C |
| ATOM | 5086 | CG | LEU | M | 11 | 36.630 | −7.843 | −14.611 | 1.00 | 25.73 | C |
| ATOM | 5087 | CD1 | LEU | M | 11 | 36.433 | −6.675 | −15.589 | 1.00 | 27.24 | C |
| ATOM | 5088 | CD2 | LEU | M | 11 | 35.293 | −8.453 | −14.156 | 1.00 | 26.81 | C |
| ATOM | 5089 | C | LEU | M | 11 | 39.662 | −7.963 | −14.295 | 1.00 | 24.41 | C |
| ATOM | 5090 | O | LEU | M | 11 | 39.503 | −8.578 | −13.235 | 1.00 | 23.51 | O |
| ATOM | 5091 | N | PRO | M | 12 | 40.454 | −6.867 | −14.398 | 1.00 | 24.21 | N |
| ATOM | 5092 | CA | PRO | M | 12 | 41.024 | −6.293 | −13.175 | 1.00 | 24.10 | C |
| ATOM | 5093 | CB | PRO | M | 12 | 42.048 | −5.259 | −13.693 | 1.00 | 24.38 | C |
| ATOM | 5094 | CG | PRO | M | 12 | 42.234 | −5.574 | −15.159 | 1.00 | 24.34 | C |
| ATOM | 5095 | CD | PRO | M | 12 | 40.934 | −6.176 | −15.608 | 1.00 | 24.62 | C |
| ATOM | 5096 | C | PRO | M | 12 | 39.916 | −5.608 | −12.378 | 1.00 | 23.74 | C |
| ATOM | 5097 | O | PRO | M | 12 | 39.115 | −4.865 | −12.946 | 1.00 | 23.83 | O |
| ATOM | 5098 | N | VAL | M | 13 | 39.828 | −5.890 | −11.085 | 1.00 | 23.32 | N |
| ATOM | 5099 | CA | VAL | M | 13 | 38.823 | −5.227 | −10.269 | 1.00 | 23.89 | C |
| ATOM | 5100 | CB | VAL | M | 13 | 37.637 | −6.148 | −9.857 | 1.00 | 24.26 | C |
| ATOM | 5101 | CG1 | VAL | M | 13 | 36.537 | −5.295 | −9.187 | 1.00 | 24.61 | C |
| ATOM | 5102 | CG2 | VAL | M | 13 | 37.051 | −6.872 | −11.060 | 1.00 | 23.25 | C |
| ATOM | 5103 | C | VAL | M | 13 | 39.452 | −4.664 | −9.017 | 1.00 | 24.58 | C |
| ATOM | 5104 | O | VAL | M | 13 | 40.320 | −5.283 | −8.402 | 1.00 | 23.72 | O |
| ATOM | 5105 | N | THR | M | 14 | 39.002 | −3.470 | −8.658 | 1.00 | 25.99 | N |
| ATOM | 5106 | CA | THR | M | 14 | 39.397 | −2.838 | −7.413 | 1.00 | 26.82 | C |
| ATOM | 5107 | CB | THR | M | 14 | 39.223 | −1.311 | −7.513 | 1.00 | 26.83 | C |
| ATOM | 5108 | OG1 | THR | M | 14 | 40.033 | −0.828 | −8.595 | 1.00 | 27.17 | O |
| ATOM | 5109 | CG2 | THR | M | 14 | 39.625 | −0.604 | −6.206 | 1.00 | 27.17 | C |
| ATOM | 5110 | C | THR | M | 14 | 38.545 | −3.442 | −6.308 | 1.00 | 27.17 | C |
| ATOM | 5111 | O | THR | M | 14 | 37.313 | −3.445 | −6.404 | 1.00 | 27.41 | O |
| ATOM | 5112 | N | PRO | M | 15 | 39.193 | −3.993 | −5.263 | 1.00 | 27.64 | N |
| ATOM | 5113 | CA | PRO | M | 15 | 38.454 | −4.566 | −4.130 | 1.00 | 27.10 | C |
| ATOM | 5114 | CB | PRO | M | 15 | 39.545 | −4.783 | −3.071 | 1.00 | 27.77 | C |
| ATOM | 5115 | CG | PRO | M | 15 | 40.755 | −4.003 | −3.572 | 1.00 | 27.55 | C |
| ATOM | 5116 | CD | PRO | M | 15 | 40.650 | −4.074 | −5.055 | 1.00 | 27.99 | C |
| ATOM | 5117 | C | PRO | M | 15 | 37.391 | −3.599 | −3.607 | 1.00 | 26.94 | C |
| ATOM | 5118 | O | PRO | M | 15 | 37.657 | −2.398 | −3.498 | 1.00 | 26.42 | O |
| ATOM | 5119 | N | GLY | M | 16 | 36.200 | −4.118 | −3.316 | 1.00 | 26.05 | N |
| ATOM | 5120 | CA | GLY | M | 16 | 35.080 | −3.301 | −2.872 | 1.00 | 25.64 | C |
| ATOM | 5121 | C | GLY | M | 16 | 34.157 | −2.868 | −4.007 | 1.00 | 25.55 | C |
| ATOM | 5122 | O | GLY | M | 16 | 33.004 | −2.511 | −3.771 | 1.00 | 25.34 | O |
| ATOM | 5123 | N | GLU | M | 17 | 34.657 | −2.904 | −5.242 | 1.00 | 24.97 | N |
| ATOM | 5124 | CA | GLU | M | 17 | 33.851 | −2.478 | −6.390 | 1.00 | 24.71 | C |
| ATOM | 5125 | CB | GLU | M | 17 | 34.726 | −1.737 | −7.405 | 1.00 | 25.12 | C |
| ATOM | 5126 | CG | GLU | M | 17 | 35.185 | −0.363 | −6.877 | 1.00 | 27.27 | C |
| ATOM | 5127 | CD | GLU | M | 17 | 36.139 | 0.365 | −7.814 | 1.00 | 32.28 | C |
| ATOM | 5128 | OE1 | GLU | M | 17 | 36.546 | −0.224 | −8.843 | 1.00 | 33.99 | O |
| ATOM | 5129 | OE2 | GLU | M | 17 | 36.489 | 1.536 | −7.516 | 1.00 | 35.16 | O |
| ATOM | 5130 | C | GLU | M | 17 | 33.074 | −3.646 | −7.024 | 1.00 | 24.17 | C |
| ATOM | 5131 | O | GLU | M | 17 | 33.414 | −4.814 | −6.796 | 1.00 | 23.20 | O |
| ATOM | 5132 | N | PRO | M | 18 | 32.002 | −3.335 | −7.781 | 1.00 | 23.69 | N |
| ATOM | 5133 | CA | PRO | M | 18 | 31.245 | −4.393 | −8.453 | 1.00 | 23.14 | C |
| ATOM | 5134 | CB | PRO | M | 18 | 29.979 | −3.685 | −8.940 | 1.00 | 23.06 | C |
| ATOM | 5135 | CG | PRO | M | 18 | 30.298 | −2.211 | −8.899 | 1.00 | 24.14 | C |
| ATOM | 5136 | CD | PRO | M | 18 | 31.329 | −2.016 | −7.843 | 1.00 | 23.90 | C |
| ATOM | 5137 | C | PRO | M | 18 | 31.989 | −5.042 | −9.629 | 1.00 | 22.90 | C |
| ATOM | 5138 | O | PRO | M | 18 | 32.928 | −4.467 | −10.182 | 1.00 | 21.68 | O |
| ATOM | 5139 | N | ALA | M | 19 | 31.576 | −6.245 | −10.000 | 1.00 | 22.55 | N |
| ATOM | 5140 | CA | ALA | M | 19 | 32.106 | −6.866 | −11.208 | 1.00 | 22.86 | C |
| ATOM | 5141 | CB | ALA | M | 19 | 33.229 | −7.832 | −10.877 | 1.00 | 22.66 | C |
| ATOM | 5142 | C | ALA | M | 19 | 30.975 | −7.568 | −11.925 | 1.00 | 23.61 | C |
| ATOM | 5143 | O | ALA | M | 19 | 30.030 | −8.049 | −11.283 | 1.00 | 23.64 | O |
| ATOM | 5144 | N | SER | M | 20 | 31.055 | −7.595 | −13.254 | 1.00 | 23.85 | N |
| ATOM | 5145 | CA | SER | M | 20 | 30.017 | −8.192 | −14.084 | 1.00 | 24.40 | C |
| ATOM | 5146 | CB | SER | M | 20 | 29.136 | −7.101 | −14.699 | 1.00 | 24.44 | C |
| ATOM | 5147 | OG | SER | M | 20 | 27.972 | −7.659 | −15.272 | 1.00 | 26.59 | O |
| ATOM | 5148 | C | SER | M | 20 | 30.674 | −9.004 | −15.180 | 1.00 | 24.59 | C |
| ATOM | 5149 | O | SER | M | 20 | 31.566 | −8.512 | −15.863 | 1.00 | 24.04 | O |
| ATOM | 5150 | N | ILE | M | 21 | 30.245 | −10.257 | −15.313 | 1.00 | 25.00 | N |
| ATOM | 5151 | CA | ILE | M | 21 | 30.777 | −11.195 | −16.303 | 1.00 | 25.52 | C |
| ATOM | 5152 | CB | ILE | M | 21 | 31.517 | −12.367 | −15.615 | 1.00 | 25.44 | C |
| ATOM | 5153 | CG1 | ILE | M | 21 | 32.718 | −11.837 | −14.830 | 1.00 | 26.80 | C |
| ATOM | 5154 | CD1 | ILE | M | 21 | 33.424 | −12.918 | −13.990 | 1.00 | 28.76 | C |
| ATOM | 5155 | CG2 | ILE | M | 21 | 31.947 | −13.432 | −16.646 | 1.00 | 27.14 | C |
| ATOM | 5156 | C | ILE | M | 21 | 29.639 | −11.766 | −17.144 | 1.00 | 24.90 | C |
| ATOM | 5157 | O | ILE | M | 21 | 28.603 | −12.148 | −16.610 | 1.00 | 25.22 | O |
| ATOM | 5158 | N | SER | M | 22 | 29.872 | −11.871 | −18.448 | 1.00 | 24.84 | N |
| ATOM | 5159 | CA | SER | M | 22 | 28.861 | −12.251 | −19.425 | 1.00 | 24.57 | C |
| ATOM | 5160 | CB | SER | M | 22 | 28.720 | −11.112 | −20.448 | 1.00 | 24.87 | C |
| ATOM | 5161 | OG | SER | M | 22 | 27.898 | −11.478 | −21.543 | 1.00 | 25.53 | O |
| ATOM | 5162 | C | SER | M | 22 | 29.203 | −13.581 | −20.125 | 1.00 | 24.26 | C |
| ATOM | 5163 | O | SER | M | 22 | 30.379 | −13.866 | −20.382 | 1.00 | 24.44 | O |
| ATOM | 5164 | N | CYS | M | 23 | 28.174 | −14.384 | −20.404 | 1.00 | 24.00 | N |
| ATOM | 5165 | CA | CYS | M | 23 | 28.298 | −15.674 | −21.098 | 1.00 | 24.29 | C |
| ATOM | 5166 | CB | CYS | M | 23 | 28.162 | −16.856 | −20.114 | 1.00 | 24.47 | C |
| ATOM | 5167 | SG | CYS | M | 23 | 28.206 | −18.583 | −20.804 | 1.00 | 27.24 | S |
| ATOM | 5168 | C | CYS | M | 23 | 27.210 | −15.724 | −22.156 | 1.00 | 24.14 | C |
| ATOM | 5169 | O | CYS | M | 23 | 26.036 | −15.522 | −21.843 | 1.00 | 23.99 | O |
| ATOM | 5170 | N | THR | M | 24 | 27.605 | −15.951 | −23.409 | 1.00 | 24.18 | N |
| ATOM | 5171 | CA | THR | M | 24 | 26.654 | −16.127 | −24.509 | 1.00 | 24.71 | C |
| ATOM | 5172 | CB | THR | M | 24 | 26.845 | −15.058 | −25.638 | 1.00 | 24.97 | C |
| ATOM | 5173 | OG1 | THR | M | 24 | 28.238 | −14.760 | −25.782 | 1.00 | 26.63 | O |
| ATOM | 5174 | CG2 | THR | M | 24 | 26.109 | −13.758 | −25.312 | 1.00 | 25.14 | C |
| ATOM | 5175 | C | THR | M | 24 | 26.797 | −17.536 | −25.098 | 1.00 | 24.84 | C |
| ATOM | 5176 | O | THR | M | 24 | 27.914 | −17.981 | −25.397 | 1.00 | 24.65 | O |
| ATOM | 5177 | N | SER | M | 25 | 25.676 | −18.240 | −25.251 | 1.00 | 24.90 | N |
| ATOM | 5178 | CA | SER | M | 25 | 25.699 | −19.570 | −25.870 | 1.00 | 25.77 | C |
| ATOM | 5179 | CB | SER | M | 25 | 24.932 | −20.591 | −25.028 | 1.00 | 25.79 | C |
| ATOM | 5180 | OG | SER | M | 25 | 23.544 | −20.303 | −24.998 | 1.00 | 26.19 | O |
| ATOM | 5181 | C | SER | M | 25 | 25.118 | −19.521 | −27.278 | 1.00 | 25.91 | C |
| ATOM | 5182 | O | SER | M | 25 | 24.133 | −18.818 | −27.510 | 1.00 | 25.84 | O |
| ATOM | 5183 | N | GLY | M | 26 | 25.718 | −20.280 | −28.199 | 1.00 | 26.16 | N |
| ATOM | 5184 | CA | GLY | M | 26 | 25.267 | −20.324 | −29.597 | 1.00 | 26.13 | C |
| ATOM | 5185 | C | GLY | M | 26 | 23.878 | −20.921 | −29.795 | 1.00 | 26.61 | C |
| ATOM | 5186 | O | GLY | M | 26 | 23.303 | −20.836 | −30.887 | 1.00 | 27.01 | O |
| ATOM | 5187 | N | GLN | M | 27 | 23.333 | −21.525 | −28.741 | 1.00 | 26.60 | N |
| ATOM | 5188 | CA | GLN | M | 27 | 22.011 | −22.147 | −28.797 | 1.00 | 26.64 | C |
| ATOM | 5189 | CB | GLN | M | 27 | 22.166 | −23.645 | −29.038 | 1.00 | 27.13 | C |
| ATOM | 5190 | CG | GLN | M | 27 | 21.020 | −24.298 | −29.786 | 1.00 | 30.45 | C |
| ATOM | 5191 | CD | GLN | M | 27 | 21.178 | −25.806 | −29.844 | 1.00 | 32.94 | C |
| ATOM | 5192 | OE1 | GLN | M | 27 | 20.488 | −26.540 | −29.131 | 1.00 | 33.46 | O |
| ATOM | 5193 | NE2 | GLN | M | 27 | 22.109 | −26.275 | −30.675 | 1.00 | 33.70 | N |
| ATOM | 5194 | C | GLN | M | 27 | 21.299 | −21.923 | −27.472 | 1.00 | 25.89 | C |
| ATOM | 5195 | O | GLN | M | 27 | 21.951 | −21.816 | −26.429 | 1.00 | 25.41 | O |
| ATOM | 5196 | N | SER | M | 27A | 19.972 | −21.855 | −27.501 | 1.00 | 24.80 | N |
| ATOM | 5197 | CA | SER | M | 27A | 19.217 | −21.701 | −26.266 | 1.00 | 24.45 | C |
| ATOM | 5198 | CB | SER | M | 27A | 17.723 | −21.639 | −26.532 | 1.00 | 24.25 | C |
| ATOM | 5199 | OG | SER | M | 27A | 17.030 | −21.788 | −25.300 | 1.00 | 24.87 | O |
| ATOM | 5200 | C | SER | M | 27A | 19.494 | −22.836 | −25.284 | 1.00 | 23.75 | C |
| ATOM | 5201 | O | SER | M | 27A | 19.505 | −24.007 | −25.671 | 1.00 | 23.68 | O |
| ATOM | 5202 | N | LEU | M | 27B | 19.694 | −22.477 | −24.015 | 1.00 | 22.98 | N |
| ATOM | 5203 | CA | LEU | M | 27B | 19.957 | −23.450 | −22.952 | 1.00 | 22.05 | C |
| ATOM | 5204 | CB | LEU | M | 27B | 21.007 | −22.897 | −21.970 | 1.00 | 21.56 | C |
| ATOM | 5205 | CG | LEU | M | 27B | 22.343 | −22.524 | −22.610 | 1.00 | 22.26 | C |
| ATOM | 5206 | CD1 | LEU | M | 27B | 23.358 | −22.092 | −21.533 | 1.00 | 21.47 | C |
| ATOM | 5207 | CD2 | LEU | M | 27B | 22.914 | −23.660 | −23.479 | 1.00 | 22.65 | C |
| ATOM | 5208 | C | LEU | M | 27B | 18.685 | −23.847 | −22.210 | 1.00 | 21.66 | C |
| ATOM | 5209 | O | LEU | M | 27B | 18.736 | −24.519 | −21.174 | 1.00 | 21.36 | O |
| ATOM | 5210 | N | VAL | M | 27C | 17.539 | −23.413 | −22.730 | 1.00 | 21.75 | N |
| ATOM | 5211 | CA | VAL | M | 27C | 16.258 | −23.778 | −22.139 | 1.00 | 21.97 | C |
| ATOM | 5212 | CB | VAL | M | 27C | 15.125 | −22.806 | −22.565 | 1.00 | 22.34 | C |
| ATOM | 5213 | CG1 | VAL | M | 27C | 13.776 | −23.276 | −22.034 | 1.00 | 23.24 | C |
| ATOM | 5214 | CG2 | VAL | M | 27C | 15.428 | −21.373 | −22.095 | 1.00 | 21.93 | C |
| ATOM | 5215 | C | VAL | M | 27C | 15.955 | −25.210 | −22.580 | 1.00 | 22.30 | C |
| ATOM | 5216 | O | VAL | M | 27C | 15.890 | −25.491 | −23.772 | 1.00 | 22.13 | O |
| ATOM | 5217 | N | HIS | M | 27D | 15.810 | −26.110 | −21.614 | 1.00 | 22.26 | N |
| ATOM | 5218 | CA | HIS | M | 27D | 15.528 | −27.524 | −21.886 | 1.00 | 22.85 | C |
| ATOM | 5219 | CB | HIS | M | 27D | 15.892 | −28.350 | −20.649 | 1.00 | 21.96 | C |
| ATOM | 5220 | CG | HIS | M | 27D | 15.892 | −29.835 | −20.865 | 1.00 | 22.36 | C |
| ATOM | 5221 | ND1 | HIS | M | 27D | 14.747 | −30.601 | −20.789 | 1.00 | 21.61 | N |
| ATOM | 5222 | CE1 | HIS | M | 27D | 15.052 | −31.870 | −20.995 | 1.00 | 22.65 | C |
| ATOM | 5223 | NE2 | HIS | M | 27D | 16.354 | −31.958 | −21.197 | 1.00 | 23.07 | N |
| ATOM | 5224 | CD2 | HIS | M | 27D | 16.905 | −30.701 | −21.110 | 1.00 | 21.22 | C |
| ATOM | 5225 | C | HIS | M | 27D | 14.042 | −27.659 | −22.225 | 1.00 | 22.85 | C |
| ATOM | 5226 | O | HIS | M | 27D | 13.240 | −26.809 | −21.826 | 1.00 | 23.52 | O |
| ATOM | 5227 | N | ILE | M | 27E | 13.664 | −28.702 | −22.964 | 1.00 | 23.15 | N |
| ATOM | 5228 | CA | ILE | M | 27E | 12.249 | −28.918 | −23.285 | 1.00 | 23.39 | C |
| ATOM | 5229 | CB | ILE | M | 27E | 12.021 | −30.099 | −24.300 | 1.00 | 23.69 | C |
| ATOM | 5230 | CG1 | ILE | M | 27E | 10.564 | −30.151 | −24.803 | 1.00 | 24.51 | C |
| ATOM | 5231 | CD1 | ILE | M | 27E | 10.045 | −28.861 | −25.475 | 1.00 | 26.53 | C |
| ATOM | 5232 | CG2 | ILE | M | 27E | 12.416 | −31.445 | −23.683 | 1.00 | 23.65 | C |
| ATOM | 5233 | C | ILE | M | 27E | 11.402 | −29.070 | −22.005 | 1.00 | 23.37 | C |
| ATOM | 5234 | O | ILE | M | 27E | 10.204 | −28.775 | −22.010 | 1.00 | 23.24 | O |
| ATOM | 5235 | N | ASN | M | 28 | 12.027 | −29.507 | −20.910 | 1.00 | 22.84 | N |
| ATOM | 5236 | CA | ASN | M | 28 | 11.335 | −29.547 | −19.620 | 1.00 | 22.76 | C |
| ATOM | 5237 | CB | ASN | M | 28 | 12.010 | −30.528 | −18.650 | 1.00 | 22.34 | C |
| ATOM | 5238 | CG | ASN | M | 28 | 13.317 | −30.000 | −18.061 | 1.00 | 21.02 | C |
| ATOM | 5239 | OD1 | ASN | M | 28 | 13.569 | −28.796 | −18.016 | 1.00 | 21.75 | O |
| ATOM | 5240 | ND2 | ASN | M | 28 | 14.157 | −30.917 | −17.601 | 1.00 | 19.05 | N |
| ATOM | 5241 | C | ASN | M | 28 | 11.129 | −28.168 | −18.972 | 1.00 | 22.74 | C |
| ATOM | 5242 | O | ASN | M | 28 | 10.572 | −28.068 | −17.881 | 1.00 | 23.63 | O |
| ATOM | 5243 | N | GLY | M | 29 | 11.596 | −27.120 | −19.639 | 1.00 | 22.96 | N |
| ATOM | 5244 | CA | GLY | M | 29 | 11.402 | −25.743 | −19.172 | 1.00 | 23.08 | C |
| ATOM | 5245 | C | GLY | M | 29 | 12.512 | −25.144 | −18.313 | 1.00 | 22.96 | C |
| ATOM | 5246 | O | GLY | M | 29 | 12.625 | −23.919 | −18.212 | 1.00 | 23.54 | O |
| ATOM | 5247 | N | ASN | M | 30 | 13.330 | −25.989 | −17.693 | 1.00 | 22.65 | N |
| ATOM | 5248 | CA | ASN | M | 30 | 14.472 | −25.515 | −16.907 | 1.00 | 22.54 | C |
| ATOM | 5249 | CB | ASN | M | 30 | 14.961 | −26.578 | −15.922 | 1.00 | 22.08 | C |
| ATOM | 5250 | CG | ASN | M | 30 | 14.045 | −26.731 | −14.720 | 1.00 | 23.65 | C |
| ATOM | 5251 | OD1 | ASN | M | 30 | 13.114 | −25.945 | −14.522 | 1.00 | 26.16 | O |
| ATOM | 5252 | ND2 | ASN | M | 30 | 14.302 | −27.752 | −13.911 | 1.00 | 24.03 | N |
| ATOM | 5253 | C | ASN | M | 30 | 15.608 | −25.076 | −17.809 | 1.00 | 22.01 | C |
| ATOM | 5254 | O | ASN | M | 30 | 15.639 | −25.427 | −18.981 | 1.00 | 22.38 | O |
| ATOM | 5255 | N | THR | M | 31 | 16.540 | −24.308 | −17.252 | 1.00 | 21.14 | N |
| ATOM | 5256 | CA | THR | M | 31 | 17.663 | −23.752 | −18.007 | 1.00 | 20.13 | C |
| ATOM | 5257 | CB | THR | M | 31 | 17.543 | −22.205 | −18.133 | 1.00 | 19.83 | C |
| ATOM | 5258 | OG1 | THR | M | 31 | 16.304 | −21.867 | −18.766 | 1.00 | 19.34 | O |
| ATOM | 5259 | CG2 | THR | M | 31 | 18.713 | −21.605 | −18.928 | 1.00 | 19.52 | C |
| ATOM | 5260 | C | THR | M | 31 | 18.943 | −24.120 | −17.275 | 1.00 | 20.21 | C |
| ATOM | 5261 | O | THR | M | 31 | 19.231 | −23.572 | −16.211 | 1.00 | 19.76 | O |
| ATOM | 5262 | N | TYR | M | 32 | 19.709 | −25.048 | −17.847 | 1.00 | 19.80 | N |
| ATOM | 5263 | CA | TYR | M | 32 | 20.839 | −25.637 | −17.133 | 1.00 | 19.50 | C |
| ATOM | 5264 | CB | TYR | M | 32 | 20.918 | −27.144 | −17.413 | 1.00 | 19.16 | C |
| ATOM | 5265 | CG | TYR | M | 32 | 19.675 | −27.896 | −16.984 | 1.00 | 19.08 | C |
| ATOM | 5266 | CD1 | TYR | M | 32 | 19.493 | −28.303 | −15.652 | 1.00 | 18.19 | C |
| ATOM | 5267 | CE1 | TYR | M | 32 | 18.326 | −28.998 | −15.260 | 1.00 | 18.93 | C |
| ATOM | 5268 | CZ | TYR | M | 32 | 17.339 | −29.278 | −16.204 | 1.00 | 19.28 | C |
| ATOM | 5269 | OH | TYR | M | 32 | 16.187 | −29.959 | −15.854 | 1.00 | 17.16 | O |
| ATOM | 5270 | CE2 | TYR | M | 32 | 17.505 | −28.886 | −17.529 | 1.00 | 19.35 | C |
| ATOM | 5271 | CD2 | TYR | M | 32 | 18.665 | −28.197 | −17.912 | 1.00 | 20.16 | C |
| ATOM | 5272 | C | TYR | M | 32 | 22.161 | −24.919 | −17.424 | 1.00 | 19.29 | C |
| ATOM | 5273 | O | TYR | M | 32 | 23.054 | −25.467 | −18.070 | 1.00 | 19.97 | O |
| ATOM | 5274 | N | LEU | M | 33 | 22.253 | −23.679 | −16.948 | 1.00 | 18.91 | N |
| ATOM | 5275 | CA | LEU | M | 33 | 23.428 | −22.824 | −17.118 | 1.00 | 18.63 | C |
| ATOM | 5276 | CB | LEU | M | 33 | 23.037 | −21.498 | −17.788 | 1.00 | 18.76 | C |
| ATOM | 5277 | CG | LEU | M | 33 | 24.105 | −20.440 | −18.144 | 1.00 | 17.87 | C |
| ATOM | 5278 | CD1 | LEU | M | 33 | 25.335 | −21.045 | −18.794 | 1.00 | 19.32 | C |
| ATOM | 5279 | CD2 | LEU | M | 33 | 24.509 | −19.584 | −16.911 | 1.00 | 20.37 | C |
| ATOM | 5280 | C | LEU | M | 33 | 23.983 | −22.558 | −15.729 | 1.00 | 18.70 | C |
| ATOM | 5281 | O | LEU | M | 33 | 23.250 | −22.091 | −14.854 | 1.00 | 18.78 | O |
| ATOM | 5282 | N | HIS | M | 34 | 25.267 | −22.858 | −15.526 | 1.00 | 17.94 | N |
| ATOM | 5283 | CA | HIS | M | 34 | 25.873 | −22.790 | −14.195 | 1.00 | 17.15 | C |
| ATOM | 5284 | CB | HIS | M | 34 | 26.155 | −24.201 | −13.657 | 1.00 | 16.53 | C |
| ATOM | 5285 | CG | HIS | M | 34 | 25.158 | −25.234 | −14.101 | 1.00 | 17.23 | C |
| ATOM | 5286 | ND1 | HIS | M | 34 | 23.835 | −25.229 | −13.698 | 1.00 | 17.38 | N |
| ATOM | 5287 | CE1 | HIS | M | 34 | 23.201 | −26.252 | −14.247 | 1.00 | 16.19 | C |
| ATOM | 5288 | NE2 | HIS | M | 34 | 24.065 | −26.926 | −14.987 | 1.00 | 16.98 | N |
| ATOM | 5289 | CD2 | HIS | M | 34 | 25.294 | −26.310 | −14.915 | 1.00 | 16.52 | C |
| ATOM | 5290 | C | HIS | M | 34 | 27.167 | −21.965 | −14.218 | 1.00 | 17.52 | C |
| ATOM | 5291 | O | HIS | M | 34 | 27.817 | −21.852 | −15.257 | 1.00 | 17.24 | O |
| ATOM | 5292 | N | TRP | M | 35 | 27.554 | −21.426 | −13.065 | 1.00 | 17.24 | N |
| ATOM | 5293 | CA | TRP | M | 35 | 28.794 | −20.654 | −12.960 | 1.00 | 17.77 | C |
| ATOM | 5294 | CB | TRP | M | 35 | 28.508 | −19.210 | −12.552 | 1.00 | 17.91 | C |
| ATOM | 5295 | CG | TRP | M | 35 | 27.834 | −18.382 | −13.569 | 1.00 | 19.37 | C |
| ATOM | 5296 | CD1 | TRP | M | 35 | 26.496 | −18.120 | −13.668 | 1.00 | 20.96 | C |
| ATOM | 5297 | NE1 | TRP | M | 35 | 26.255 | −17.273 | −14.731 | 1.00 | 18.96 | N |
| ATOM | 5298 | CE2 | TRP | M | 35 | 27.446 | −16.973 | −15.330 | 1.00 | 20.53 | C |
| ATOM | 5299 | CD2 | TRP | M | 35 | 28.464 | −17.650 | −14.619 | 1.00 | 20.11 | C |
| ATOM | 5300 | CE3 | TRP | M | 35 | 29.794 | −17.513 | −15.040 | 1.00 | 21.15 | C |
| ATOM | 5301 | CZ3 | TRP | M | 35 | 30.063 | −16.707 | −16.135 | 1.00 | 22.30 | C |
| ATOM | 5302 | CH2 | TRP | M | 35 | 29.031 | −16.036 | −16.814 | 1.00 | 21.44 | C |
| ATOM | 5303 | CZ2 | TRP | M | 35 | 27.718 | −16.154 | −16.433 | 1.00 | 19.67 | C |
| ATOM | 5304 | C | TRP | M | 35 | 29.746 | −21.253 | −11.933 | 1.00 | 17.36 | C |
| ATOM | 5305 | O | TRP | M | 35 | 29.331 | −21.624 | −10.834 | 1.00 | 16.56 | O |
| ATOM | 5306 | N | TYR | M | 36 | 31.017 | −21.321 | −12.318 | 1.00 | 16.79 | N |
| ATOM | 5307 | CA | TYR | M | 36 | 32.111 | −21.843 | −11.490 | 1.00 | 16.57 | C |
| ATOM | 5308 | CB | TYR | M | 36 | 32.723 | −23.086 | −12.152 | 1.00 | 16.32 | C |
| ATOM | 5309 | CG | TYR | M | 36 | 31.810 | −24.283 | −12.147 | 1.00 | 18.42 | C |
| ATOM | 5310 | CD1 | TYR | M | 36 | 31.994 | −25.318 | −11.233 | 1.00 | 18.95 | C |
| ATOM | 5311 | CE1 | TYR | M | 36 | 31.133 | −26.413 | −11.209 | 1.00 | 18.71 | C |
| ATOM | 5312 | CZ | TYR | M | 36 | 30.087 | −26.468 | −12.114 | 1.00 | 19.38 | C |
| ATOM | 5313 | OH | TYR | M | 36 | 29.226 | −27.521 | −12.109 | 1.00 | 21.02 | O |
| ATOM | 5314 | CE2 | TYR | M | 36 | 29.889 | −25.460 | −13.035 | 1.00 | 18.65 | C |
| ATOM | 5315 | CD2 | TYR | M | 36 | 30.749 | −24.372 | −13.044 | 1.00 | 19.24 | C |
| ATOM | 5316 | C | TYR | M | 36 | 33.207 | −20.783 | −11.321 | 1.00 | 16.50 | C |
| ATOM | 5317 | O | TYR | M | 36 | 33.392 | −19.912 | −12.187 | 1.00 | 16.43 | O |
| ATOM | 5318 | N | LEU | M | 37 | 33.922 | −20.876 | −10.207 | 1.00 | 16.72 | N |
| ATOM | 5319 | CA | LEU | M | 37 | 35.148 | −20.141 | −9.989 | 1.00 | 17.77 | C |
| ATOM | 5320 | CB | LEU | M | 37 | 35.016 | −19.210 | −8.758 | 1.00 | 17.12 | C |
| ATOM | 5321 | CG | LEU | M | 37 | 36.295 | −18.668 | −8.116 | 1.00 | 17.13 | C |
| ATOM | 5322 | CD1 | LEU | M | 37 | 37.105 | −17.818 | −9.117 | 1.00 | 17.67 | C |
| ATOM | 5323 | CD2 | LEU | M | 37 | 35.966 | −17.870 | −6.864 | 1.00 | 18.19 | C |
| ATOM | 5324 | C | LEU | M | 37 | 36.257 | −21.172 | −9.739 | 1.00 | 18.38 | C |
| ATOM | 5325 | O | LEU | M | 37 | 36.106 | −22.086 | −8.907 | 1.00 | 16.72 | O |
| ATOM | 5326 | N | GLN | M | 38 | 37.358 | −21.022 | −10.471 | 1.00 | 19.04 | N |
| ATOM | 5327 | CA | GLN | M | 38 | 38.518 | −21.874 | −10.260 | 1.00 | 20.47 | C |
| ATOM | 5328 | CB | GLN | M | 38 | 38.982 | −22.536 | −11.558 | 1.00 | 19.49 | C |
| ATOM | 5329 | CG | GLN | M | 38 | 40.166 | −23.481 | −11.341 | 1.00 | 20.18 | C |
| ATOM | 5330 | CD | GLN | M | 38 | 40.584 | −24.206 | −12.606 | 1.00 | 20.21 | C |
| ATOM | 5331 | OE1 | GLN | M | 38 | 40.533 | −23.639 | −13.691 | 1.00 | 19.87 | O |
| ATOM | 5332 | NE2 | GLN | M | 38 | 41.005 | −25.475 | −12.465 | 1.00 | 19.85 | N |
| ATOM | 5333 | C | GLN | M | 38 | 39.617 | −21.016 | −9.670 | 1.00 | 22.09 | C |
| ATOM | 5334 | O | GLN | M | 38 | 40.196 | −20.151 | −10.353 | 1.00 | 21.27 | O |
| ATOM | 5335 | N | LYS | M | 39 | 39.862 | −21.231 | −8.388 | 1.00 | 24.23 | N |
| ATOM | 5336 | CA | LYS | M | 39 | 40.895 | −20.499 | −7.677 | 1.00 | 27.47 | C |
| ATOM | 5337 | CB | LYS | M | 39 | 40.634 | −20.500 | −6.152 | 1.00 | 26.67 | C |
| ATOM | 5338 | CG | LYS | M | 39 | 39.474 | −19.565 | −5.748 | 1.00 | 29.40 | C |
| ATOM | 5339 | CD | LYS | M | 39 | 39.184 | −19.537 | −4.242 | 1.00 | 30.86 | C |
| ATOM | 5340 | CE | LYS | M | 39 | 37.823 | −18.890 | −3.966 | 1.00 | 33.07 | C |
| ATOM | 5341 | NZ | LYS | M | 39 | 37.420 | −18.871 | −2.521 | 1.00 | 30.92 | N |
| ATOM | 5342 | C | LYS | M | 39 | 42.203 | −21.153 | −8.073 | 1.00 | 28.52 | C |
| ATOM | 5343 | O | LYS | M | 39 | 42.227 | −22.345 | −8.360 | 1.00 | 29.19 | O |
| ATOM | 5344 | N | PRO | M | 40 | 43.299 | −20.377 | −8.147 | 1.00 | 30.30 | N |
| ATOM | 5345 | CA | PRO | M | 40 | 44.510 | −20.985 | −8.723 | 1.00 | 30.35 | C |
| ATOM | 5346 | CB | PRO | M | 40 | 45.510 | −19.814 | −8.762 | 1.00 | 30.76 | C |
| ATOM | 5347 | CG | PRO | M | 40 | 45.002 | −18.836 | −7.747 | 1.00 | 31.31 | C |
| ATOM | 5348 | CD | PRO | M | 40 | 43.504 | −18.960 | −7.780 | 1.00 | 30.89 | C |
| ATOM | 5349 | C | PRO | M | 40 | 45.026 | −22.184 | −7.914 | 1.00 | 30.35 | C |
| ATOM | 5350 | O | PRO | M | 40 | 44.934 | −22.188 | −6.683 | 1.00 | 30.82 | O |
| ATOM | 5351 | N | GLY | M | 41 | 45.505 | −23.213 | −8.608 | 1.00 | 29.77 | N |
| ATOM | 5352 | CA | GLY | M | 41 | 45.902 | −24.469 | −7.967 | 1.00 | 29.38 | C |
| ATOM | 5353 | C | GLY | M | 41 | 44.777 | −25.478 | −7.691 | 1.00 | 28.61 | C |
| ATOM | 5354 | O | GLY | M | 41 | 45.033 | −26.631 | −7.377 | 1.00 | 28.84 | O |
| ATOM | 5355 | N | GLN | M | 42 | 43.528 | −25.049 | −7.819 | 1.00 | 27.67 | N |
| ATOM | 5356 | CA | GLN | M | 42 | 42.396 | −25.862 | −7.401 | 1.00 | 26.23 | C |
| ATOM | 5357 | CB | GLN | M | 42 | 41.566 | −25.066 | −6.404 | 1.00 | 26.84 | C |
| ATOM | 5358 | CG | GLN | M | 42 | 42.251 | −24.840 | −5.063 | 1.00 | 30.06 | C |
| ATOM | 5359 | CD | GLN | M | 42 | 41.547 | −23.761 | −4.265 | 1.00 | 35.55 | C |
| ATOM | 5360 | OE1 | GLN | M | 42 | 40.305 | −23.657 | −4.284 | 1.00 | 36.89 | O |
| ATOM | 5361 | NE2 | GLN | M | 42 | 42.329 | −22.945 | −3.556 | 1.00 | 37.16 | N |
| ATOM | 5362 | C | GLN | M | 42 | 41.506 | −26.315 | −8.559 | 1.00 | 24.58 | C |
| ATOM | 5363 | O | GLN | M | 42 | 41.619 | −25.804 | −9.666 | 1.00 | 24.07 | O |
| ATOM | 5364 | N | SER | M | 43 | 40.622 | −27.280 | −8.290 | 1.00 | 22.27 | N |
| ATOM | 5365 | CA | SER | M | 43 | 39.550 | −27.618 | −9.214 | 1.00 | 21.00 | C |
| ATOM | 5366 | CB | SER | M | 43 | 38.893 | −28.960 | −8.822 | 1.00 | 21.71 | C |
| ATOM | 5367 | OG | SER | M | 43 | 38.217 | −28.856 | −7.577 | 1.00 | 21.38 | O |
| ATOM | 5368 | C | SER | M | 43 | 38.528 | −26.480 | −9.244 | 1.00 | 20.27 | C |
| ATOM | 5369 | O | SER | M | 43 | 38.509 | −25.653 | −8.319 | 1.00 | 19.15 | O |
| ATOM | 5370 | N | PRO | M | 44 | 37.693 | −26.404 | −10.310 | 1.00 | 19.07 | N |
| ATOM | 5371 | CA | PRO | M | 44 | 36.608 | −25.422 | −10.276 | 1.00 | 19.09 | C |
| ATOM | 5372 | CB | PRO | M | 44 | 35.870 | −25.640 | −11.605 | 1.00 | 18.17 | C |
| ATOM | 5373 | CG | PRO | M | 44 | 36.844 | −26.356 | −12.497 | 1.00 | 19.23 | C |
| ATOM | 5374 | CD | PRO | M | 44 | 37.700 | −27.184 | −11.564 | 1.00 | 19.44 | C |
| ATOM | 5375 | C | PRO | M | 44 | 35.634 | −25.715 | −9.124 | 1.00 | 19.19 | C |
| ATOM | 5376 | O | PRO | M | 44 | 35.433 | −26.874 | −8.757 | 1.00 | 19.23 | O |
| ATOM | 5377 | N | LYS | M | 45 | 35.008 | −24.666 | −8.606 | 1.00 | 19.58 | N |
| ATOM | 5378 | CA | LYS | M | 45 | 34.027 | −24.788 | −7.530 | 1.00 | 19.62 | C |
| ATOM | 5379 | CB | LYS | M | 45 | 34.507 | −24.034 | −6.275 | 1.00 | 20.56 | C |
| ATOM | 5380 | CG | LYS | M | 45 | 35.571 | −24.757 | −5.462 | 1.00 | 25.00 | C |
| ATOM | 5381 | CD | LYS | M | 45 | 35.533 | −24.309 | −3.985 | 1.00 | 30.39 | C |
| ATOM | 5382 | CE | LYS | M | 45 | 35.818 | −25.491 | −3.037 | 1.00 | 33.41 | C |
| ATOM | 5383 | NZ | LYS | M | 45 | 34.658 | −26.457 | −2.853 | 1.00 | 32.77 | N |
| ATOM | 5384 | C | LYS | M | 45 | 32.746 | −24.158 | −8.035 | 1.00 | 18.81 | C |
| ATOM | 5385 | O | LYS | M | 45 | 32.795 | −23.058 | −8.616 | 1.00 | 18.19 | O |
| ATOM | 5386 | N | LEU | M | 46 | 31.610 | −24.825 | −7.803 | 1.00 | 17.44 | N |
| ATOM | 5387 | CA | LEU | M | 46 | 30.305 | −24.306 | −8.212 | 1.00 | 16.73 | C |
| ATOM | 5388 | CB | LEU | M | 46 | 29.255 | −25.432 | −8.205 | 1.00 | 17.15 | C |
| ATOM | 5389 | CG | LEU | M | 46 | 27.784 | −25.134 | −8.543 | 1.00 | 16.18 | C |
| ATOM | 5390 | CD1 | LEU | M | 46 | 27.585 | −24.713 | −9.992 | 1.00 | 13.62 | C |
| ATOM | 5391 | CD2 | LEU | M | 46 | 26.966 | −26.372 | −8.256 | 1.00 | 15.44 | C |
| ATOM | 5392 | C | LEU | M | 46 | 29.832 | −23.113 | −7.368 | 1.00 | 16.90 | C |
| ATOM | 5393 | O | LEU | M | 46 | 29.825 | −23.156 | −6.123 | 1.00 | 17.78 | O |
| ATOM | 5394 | N | LEU | M | 47 | 29.408 | −22.066 | −8.060 | 1.00 | 16.08 | N |
| ATOM | 5395 | CA | LEU | M | 47 | 28.843 | −20.882 | −7.422 | 1.00 | 16.92 | C |
| ATOM | 5396 | CB | LEU | M | 47 | 29.443 | −19.617 | −8.040 | 1.00 | 16.26 | C |
| ATOM | 5397 | CG | LEU | M | 47 | 30.961 | −19.443 | −7.922 | 1.00 | 19.62 | C |
| ATOM | 5398 | CD1 | LEU | M | 47 | 31.358 | −18.129 | −8.612 | 1.00 | 20.74 | C |
| ATOM | 5399 | CD2 | LEU | M | 47 | 31.457 | −19.491 | −6.458 | 1.00 | 19.82 | C |
| ATOM | 5400 | C | LEU | M | 47 | 27.326 | −20.812 | −7.554 | 1.00 | 16.35 | C |
| ATOM | 5401 | O | LEU | M | 47 | 26.624 | −20.552 | −6.582 | 1.00 | 15.97 | O |
| ATOM | 5402 | N | ILE | M | 48 | 26.845 | −21.010 | −8.778 | 1.00 | 16.72 | N |
| ATOM | 5403 | CA | ILE | M | 48 | 25.443 | −20.784 | −9.133 | 1.00 | 17.30 | C |
| ATOM | 5404 | CB | ILE | M | 48 | 25.219 | −19.391 | −9.755 | 1.00 | 18.12 | C |
| ATOM | 5405 | CG1 | ILE | M | 48 | 25.524 | −18.262 | −8.754 | 1.00 | 17.91 | C |
| ATOM | 5406 | CD1 | ILE | M | 48 | 25.602 | −16.876 | −9.422 | 1.00 | 20.03 | C |
| ATOM | 5407 | CG2 | ILE | M | 48 | 23.777 | −19.259 | −10.358 | 1.00 | 17.63 | C |
| ATOM | 5408 | C | ILE | M | 48 | 25.021 | −21.839 | −10.143 | 1.00 | 17.65 | C |
| ATOM | 5409 | O | ILE | M | 48 | 25.714 | −22.059 | −11.141 | 1.00 | 17.87 | O |
| ATOM | 5410 | N | TYR | M | 49 | 23.915 | −22.528 | −9.843 | 1.00 | 18.29 | N |
| ATOM | 5411 | CA | TYR | M | 49 | 23.321 | −23.507 | −10.747 | 1.00 | 18.06 | C |
| ATOM | 5412 | CB | TYR | M | 49 | 23.196 | −24.893 | −10.079 | 1.00 | 18.32 | C |
| ATOM | 5413 | CG | TYR | M | 49 | 22.219 | −24.981 | −8.917 | 1.00 | 19.65 | C |
| ATOM | 5414 | CD1 | TYR | M | 49 | 20.897 | −25.403 | −9.114 | 1.00 | 21.21 | C |
| ATOM | 5415 | CE1 | TYR | M | 49 | 19.992 | −25.497 | −8.036 | 1.00 | 21.54 | C |
| ATOM | 5416 | CZ | TYR | M | 49 | 20.428 | −25.183 | −6.754 | 1.00 | 23.90 | C |
| ATOM | 5417 | OH | TYR | M | 49 | 19.564 | −25.255 | −5.687 | 1.00 | 23.43 | O |
| ATOM | 5418 | CE2 | TYR | M | 49 | 21.734 | −24.769 | −6.540 | 1.00 | 23.19 | C |
| ATOM | 5419 | CD2 | TYR | M | 49 | 22.625 | −24.680 | −7.621 | 1.00 | 20.68 | C |
| ATOM | 5420 | C | TYR | M | 49 | 21.965 | −23.008 | −11.235 | 1.00 | 18.29 | C |
| ATOM | 5421 | O | TYR | M | 49 | 21.299 | −22.228 | −10.540 | 1.00 | 16.98 | O |
| ATOM | 5422 | N | LYS | M | 50 | 21.571 | −23.477 | −12.425 | 1.00 | 17.38 | N |
| ATOM | 5423 | CA | LYS | M | 50 | 20.317 | −23.083 | −13.087 | 1.00 | 17.60 | C |
| ATOM | 5424 | CB | LYS | M | 50 | 19.121 | −23.866 | −12.540 | 1.00 | 17.05 | C |
| ATOM | 5425 | CG | LYS | M | 50 | 19.050 | −25.311 | −13.025 | 1.00 | 18.22 | C |
| ATOM | 5426 | CD | LYS | M | 50 | 17.686 | −25.936 | −12.768 | 1.00 | 17.66 | C |
| ATOM | 5427 | CE | LYS | M | 50 | 17.450 | −26.154 | −11.289 | 1.00 | 17.56 | C |
| ATOM | 5428 | NZ | LYS | M | 50 | 16.182 | −26.886 | −11.055 | 1.00 | 18.52 | N |
| ATOM | 5429 | C | LYS | M | 50 | 20.066 | −21.575 | −13.074 | 1.00 | 17.42 | C |
| ATOM | 5430 | O | LYS | M | 50 | 19.060 | −21.098 | −12.546 | 1.00 | 16.39 | O |
| ATOM | 5431 | N | VAL | M | 51 | 21.021 | −20.847 | −13.664 | 1.00 | 17.74 | N |
| ATOM | 5432 | CA | VAL | M | 51 | 20.968 | −19.391 | −13.845 | 1.00 | 17.88 | C |
| ATOM | 5433 | CB | VAL | M | 51 | 19.691 | −18.936 | −14.620 | 1.00 | 18.11 | C |
| ATOM | 5434 | CG1 | VAL | M | 51 | 19.715 | −17.430 | −14.900 | 1.00 | 16.68 | C |
| ATOM | 5435 | CG2 | VAL | M | 51 | 19.597 | −19.704 | −15.928 | 1.00 | 17.20 | C |
| ATOM | 5436 | C | VAL | M | 51 | 21.182 | −18.542 | −12.595 | 1.00 | 17.92 | C |
| ATOM | 5437 | O | VAL | M | 51 | 21.993 | −17.624 | −12.619 | 1.00 | 17.85 | O |
| ATOM | 5438 | N | SER | M | 52 | 20.482 | −18.841 | −11.504 | 1.00 | 18.58 | N |
| ATOM | 5439 | CA | SER | M | 52 | 20.430 | −17.883 | −10.410 | 1.00 | 19.23 | C |
| ATOM | 5440 | CB | SER | M | 52 | 19.251 | −16.946 | −10.643 | 1.00 | 19.83 | C |
| ATOM | 5441 | OG | SER | M | 52 | 18.071 | −17.712 | −10.753 | 1.00 | 20.76 | O |
| ATOM | 5442 | C | SER | M | 52 | 20.331 | −18.495 | −9.015 | 1.00 | 19.56 | C |
| ATOM | 5443 | O | SER | M | 52 | 20.095 | −17.771 | −8.037 | 1.00 | 19.62 | O |
| ATOM | 5444 | N | ASN | M | 53 | 20.490 | −19.814 | −8.908 | 1.00 | 18.67 | N |
| ATOM | 5445 | CA | ASN | M | 53 | 20.400 | −20.451 | −7.598 | 1.00 | 19.30 | C |
| ATOM | 5446 | CB | ASN | M | 53 | 19.798 | −21.861 | −7.690 | 1.00 | 19.26 | C |
| ATOM | 5447 | CG | ASN | M | 53 | 18.438 | −21.877 | −8.342 | 1.00 | 21.65 | C |
| ATOM | 5448 | OD1 | ASN | M | 53 | 18.318 | −22.037 | −9.563 | 1.00 | 23.44 | O |
| ATOM | 5449 | ND2 | ASN | M | 53 | 17.402 | −21.710 | −7.538 | 1.00 | 22.00 | N |
| ATOM | 5450 | C | ASN | M | 53 | 21.794 | −20.533 | −7.011 | 1.00 | 18.64 | C |
| ATOM | 5451 | O | ASN | M | 53 | 22.671 | −21.185 | −7.598 | 1.00 | 18.91 | O |
| ATOM | 5452 | N | LEU | M | 54 | 21.995 | −19.879 | −5.870 | 1.00 | 17.81 | N |
| ATOM | 5453 | CA | LEU | M | 54 | 23.256 | −19.963 | −5.139 | 1.00 | 18.32 | C |
| ATOM | 5454 | CB | LEU | M | 54 | 23.293 | −18.924 | −4.001 | 1.00 | 18.32 | C |
| ATOM | 5455 | CG | LEU | M | 54 | 23.345 | −17.440 | −4.394 | 1.00 | 19.58 | C |
| ATOM | 5456 | CD1 | LEU | M | 54 | 23.339 | −16.518 | −3.154 | 1.00 | 18.88 | C |
| ATOM | 5457 | CD2 | LEU | M | 54 | 24.580 | −17.147 | −5.247 | 1.00 | 20.64 | C |
| ATOM | 5458 | C | LEU | M | 54 | 23.460 | −21.371 | −4.603 | 1.00 | 18.41 | C |
| ATOM | 5459 | O | LEU | M | 54 | 22.558 | −21.932 | −3.982 | 1.00 | 18.10 | O |
| ATOM | 5460 | N | PHE | M | 55 | 24.632 | −21.950 | −4.862 | 1.00 | 18.68 | N |
| ATOM | 5461 | CA | PHE | M | 55 | 24.970 | −23.266 | −4.316 | 1.00 | 19.08 | C |
| ATOM | 5462 | CB | PHE | M | 55 | 26.147 | −23.890 | −5.106 | 1.00 | 18.71 | C |
| ATOM | 5463 | CG | PHE | M | 55 | 26.496 | −25.302 | −4.699 | 1.00 | 18.74 | C |
| ATOM | 5464 | CD1 | PHE | M | 55 | 25.640 | −26.360 | −4.974 | 1.00 | 18.10 | C |
| ATOM | 5465 | CE1 | PHE | M | 55 | 25.972 | −27.674 | −4.600 | 1.00 | 20.10 | C |
| ATOM | 5466 | CZ | PHE | M | 55 | 27.184 | −27.935 | −3.944 | 1.00 | 19.27 | C |
| ATOM | 5467 | CE2 | PHE | M | 55 | 28.051 | −26.884 | −3.657 | 1.00 | 21.36 | C |
| ATOM | 5468 | CD2 | PHE | M | 55 | 27.704 | −25.570 | −4.045 | 1.00 | 20.02 | C |
| ATOM | 5469 | C | PHE | M | 55 | 25.257 | −23.110 | −2.817 | 1.00 | 19.78 | C |
| ATOM | 5470 | O | PHE | M | 55 | 25.692 | −22.037 | −2.378 | 1.00 | 18.98 | O |
| ATOM | 5471 | N | SER | M | 56 | 24.959 | −24.143 | −2.023 | 1.00 | 20.27 | N |
| ATOM | 5472 | CA | SER | M | 56 | 25.138 | −24.057 | −0.567 | 1.00 | 21.98 | C |
| ATOM | 5473 | CB | SER | M | 56 | 24.945 | −25.428 | 0.088 | 1.00 | 23.10 | C |
| ATOM | 5474 | OG | SER | M | 56 | 26.005 | −26.294 | −0.311 | 1.00 | 27.59 | O |
| ATOM | 5475 | C | SER | M | 56 | 26.503 | −23.484 | −0.179 | 1.00 | 21.75 | C |
| ATOM | 5476 | O | SER | M | 56 | 27.552 | −23.916 | −0.682 | 1.00 | 21.81 | O |
| ATOM | 5477 | N | GLY | M | 57 | 26.479 | −22.483 | 0.696 | 1.00 | 21.37 | N |
| ATOM | 5478 | CA | GLY | M | 57 | 27.697 | −21.884 | 1.226 | 1.00 | 21.36 | C |
| ATOM | 5479 | C | GLY | M | 57 | 28.205 | −20.670 | 0.459 | 1.00 | 20.84 | C |
| ATOM | 5480 | O | GLY | M | 57 | 29.071 | −19.962 | 0.937 | 1.00 | 21.44 | O |
| ATOM | 5481 | N | VAL | M | 58 | 27.684 | −20.424 | −0.738 | 1.00 | 20.02 | N |
| ATOM | 5482 | CA | VAL | M | 58 | 28.175 | −19.313 | −1.562 | 1.00 | 19.11 | C |
| ATOM | 5483 | CB | VAL | M | 58 | 27.855 | −19.579 | −3.051 | 1.00 | 19.48 | C |
| ATOM | 5484 | CG1 | VAL | M | 58 | 28.270 | −18.405 | −3.961 | 1.00 | 18.53 | C |
| ATOM | 5485 | CG2 | VAL | M | 58 | 28.554 | −20.868 | −3.485 | 1.00 | 19.24 | C |
| ATOM | 5486 | C | VAL | M | 58 | 27.562 | −17.998 | −1.072 | 1.00 | 18.86 | C |
| ATOM | 5487 | O | VAL | M | 58 | 26.362 | −17.935 | −0.879 | 1.00 | 17.95 | O |
| ATOM | 5488 | N | PRO | M | 59 | 28.385 | −16.951 | −0.861 | 1.00 | 18.98 | N |
| ATOM | 5489 | CA | PRO | M | 59 | 27.816 | −15.702 | −0.327 | 1.00 | 19.11 | C |
| ATOM | 5490 | CB | PRO | M | 59 | 29.047 | −14.844 | 0.010 | 1.00 | 19.41 | C |
| ATOM | 5491 | CG | PRO | M | 59 | 30.173 | −15.442 | −0.734 | 1.00 | 19.68 | C |
| ATOM | 5492 | CD | PRO | M | 59 | 29.856 | −16.898 | −0.966 | 1.00 | 19.00 | C |
| ATOM | 5493 | C | PRO | M | 59 | 26.888 | −14.969 | −1.311 | 1.00 | 19.32 | C |
| ATOM | 5494 | O | PRO | M | 59 | 27.038 | −15.103 | −2.543 | 1.00 | 17.85 | O |
| ATOM | 5495 | N | ASP | M | 60 | 25.943 | −14.214 | −0.747 | 1.00 | 18.79 | N |
| ATOM | 5496 | CA | ASP | M | 60 | 24.890 | −13.546 | −1.511 | 1.00 | 19.00 | C |
| ATOM | 5497 | CB | ASP | M | 60 | 23.652 | −13.213 | −0.636 | 1.00 | 18.64 | C |
| ATOM | 5498 | CG | ASP | M | 60 | 23.951 | −12.233 | 0.514 | 1.00 | 18.48 | C |
| ATOM | 5499 | OD1 | ASP | M | 60 | 24.946 | −11.477 | 0.474 | 1.00 | 17.72 | O |
| ATOM | 5500 | OD2 | ASP | M | 60 | 23.152 | −12.198 | 1.477 | 1.00 | 18.54 | O |
| ATOM | 5501 | C | ASP | M | 60 | 25.406 | −12.310 | −2.242 | 1.00 | 18.85 | C |
| ATOM | 5502 | O | ASP | M | 60 | 24.636 | −11.614 | −2.893 | 1.00 | 18.28 | O |
| ATOM | 5503 | N | ARG | M | 61 | 26.705 | −12.042 | −2.133 | 1.00 | 18.50 | N |
| ATOM | 5504 | CA | ARG | M | 61 | 27.332 | −11.050 | −3.008 | 1.00 | 19.07 | C |
| ATOM | 5505 | CB | ARG | M | 61 | 28.693 | −10.603 | −2.459 | 1.00 | 19.53 | C |
| ATOM | 5506 | CG | ARG | M | 61 | 29.833 | −11.590 | −2.629 | 1.00 | 21.82 | C |
| ATOM | 5507 | CD | ARG | M | 61 | 31.179 | −10.894 | −2.308 | 1.00 | 25.35 | C |
| ATOM | 5508 | NE | ARG | M | 61 | 32.295 | −11.840 | −2.301 | 1.00 | 27.27 | N |
| ATOM | 5509 | CZ | ARG | M | 61 | 32.580 | −12.640 | −1.277 | 1.00 | 27.09 | C |
| ATOM | 5510 | NH1 | ARG | M | 61 | 31.849 | −12.590 | −0.177 | 1.00 | 26.62 | N |
| ATOM | 5511 | NH2 | ARG | M | 61 | 33.599 | −13.489 | −1.346 | 1.00 | 28.81 | N |
| ATOM | 5512 | C | ARG | M | 61 | 27.405 | −11.505 | −4.489 | 1.00 | 18.53 | C |
| ATOM | 5513 | O | ARG | M | 61 | 27.516 | −10.675 | −5.397 | 1.00 | 18.14 | O |
| ATOM | 5514 | N | PHE | M | 62 | 27.332 | −12.819 | −4.732 | 1.00 | 18.00 | N |
| ATOM | 5515 | CA | PHE | M | 62 | 27.282 | −13.343 | −6.098 | 1.00 | 18.16 | C |
| ATOM | 5516 | CB | PHE | M | 62 | 27.940 | −14.733 | −6.193 | 1.00 | 18.26 | C |
| ATOM | 5517 | CG | PHE | M | 62 | 29.397 | −14.728 | −5.902 | 1.00 | 19.71 | C |
| ATOM | 5518 | CD1 | PHE | M | 62 | 30.325 | −14.562 | −6.935 | 1.00 | 19.85 | C |
| ATOM | 5519 | CE1 | PHE | M | 62 | 31.682 | −14.529 | −6.667 | 1.00 | 19.42 | C |
| ATOM | 5520 | CZ | PHE | M | 62 | 32.136 | −14.664 | −5.355 | 1.00 | 21.27 | C |
| ATOM | 5521 | CE2 | PHE | M | 62 | 31.228 | −14.809 | −4.315 | 1.00 | 19.26 | C |
| ATOM | 5522 | CD2 | PHE | M | 62 | 29.859 | −14.850 | −4.595 | 1.00 | 19.38 | C |
| ATOM | 5523 | C | PHE | M | 62 | 25.820 | −13.473 | −6.470 | 1.00 | 18.25 | C |
| ATOM | 5524 | O | PHE | M | 62 | 25.005 | −13.884 | −5.638 | 1.00 | 18.21 | O |
| ATOM | 5525 | N | SER | M | 63 | 25.479 | −13.110 | −7.703 | 1.00 | 18.40 | N |
| ATOM | 5526 | CA | SER | M | 63 | 24.124 | −13.329 | −8.233 | 1.00 | 18.43 | C |
| ATOM | 5527 | CB | SER | M | 63 | 23.235 | −12.097 | −8.043 | 1.00 | 19.04 | C |
| ATOM | 5528 | OG | SER | M | 63 | 23.770 | −10.952 | −8.695 | 1.00 | 19.20 | O |
| ATOM | 5529 | C | SER | M | 63 | 24.229 | −13.652 | −9.719 | 1.00 | 18.95 | C |
| ATOM | 5530 | O | SER | M | 63 | 25.197 | −13.266 | −10.384 | 1.00 | 18.96 | O |
| ATOM | 5531 | N | GLY | M | 64 | 23.240 | −14.362 | −10.239 | 1.00 | 19.09 | N |
| ATOM | 5532 | CA | GLY | M | 64 | 23.244 | −14.719 | −11.651 | 1.00 | 19.94 | C |
| ATOM | 5533 | C | GLY | M | 64 | 21.896 | −14.400 | −12.237 | 1.00 | 20.14 | C |
| ATOM | 5534 | O | GLY | M | 64 | 20.884 | −14.450 | −11.541 | 1.00 | 20.25 | O |
| ATOM | 5535 | N | SER | M | 65 | 21.877 | −14.058 | −13.518 | 1.00 | 20.47 | N |
| ATOM | 5536 | CA | SER | M | 65 | 20.622 | −13.761 | −14.195 | 1.00 | 21.01 | C |
| ATOM | 5537 | CB | SER | M | 65 | 20.216 | −12.302 | −13.951 | 1.00 | 21.43 | C |
| ATOM | 5538 | OG | SER | M | 65 | 21.179 | −11.410 | −14.494 | 1.00 | 21.07 | O |
| ATOM | 5539 | C | SER | M | 65 | 20.770 | −14.038 | −15.688 | 1.00 | 21.47 | C |
| ATOM | 5540 | O | SER | M | 65 | 21.854 | −14.404 | −16.170 | 1.00 | 20.75 | O |
| ATOM | 5541 | N | GLY | M | 66 | 19.675 | −13.880 | −16.416 | 1.00 | 22.09 | N |
| ATOM | 5542 | CA | GLY | M | 66 | 19.713 | −14.027 | −17.869 | 1.00 | 22.56 | C |
| ATOM | 5543 | C | GLY | M | 66 | 18.689 | −15.057 | −18.271 | 1.00 | 22.85 | C |
| ATOM | 5544 | O | GLY | M | 66 | 17.966 | −15.572 | −17.417 | 1.00 | 23.27 | O |
| ATOM | 5545 | N | SER | M | 67 | 18.622 | −15.350 | −19.569 | 1.00 | 23.05 | N |
| ATOM | 5546 | CA | SER | M | 67 | 17.667 | −16.316 | −20.117 | 1.00 | 22.88 | C |
| ATOM | 5547 | CB | SER | M | 67 | 16.269 | −15.703 | −20.148 | 1.00 | 22.84 | C |
| ATOM | 5548 | OG | SER | M | 67 | 16.275 | −14.563 | −20.990 | 1.00 | 22.98 | O |
| ATOM | 5549 | C | SER | M | 67 | 18.065 | −16.708 | −21.541 | 1.00 | 22.87 | C |
| ATOM | 5550 | O | SER | M | 67 | 18.765 | −15.955 | −22.232 | 1.00 | 22.73 | O |
| ATOM | 5551 | N | GLY | M | 68 | 17.607 | −17.882 | −21.970 | 1.00 | 22.42 | N |
| ATOM | 5552 | CA | GLY | M | 68 | 17.815 | −18.348 | −23.335 | 1.00 | 22.53 | C |
| ATOM | 5553 | C | GLY | M | 68 | 19.273 | −18.536 | −23.696 | 1.00 | 22.73 | C |
| ATOM | 5554 | O | GLY | M | 68 | 19.861 | −19.587 | −23.414 | 1.00 | 22.66 | O |
| ATOM | 5555 | N | THR | M | 69 | 19.857 | −17.503 | −24.300 | 1.00 | 22.49 | N |
| ATOM | 5556 | CA | THR | M | 69 | 21.234 | −17.550 | −24.777 | 1.00 | 23.07 | C |
| ATOM | 5557 | CB | THR | M | 69 | 21.313 | −17.206 | −26.276 | 1.00 | 22.94 | C |
| ATOM | 5558 | OG1 | THR | M | 69 | 20.559 | −16.014 | −26.519 | 1.00 | 23.76 | O |
| ATOM | 5559 | CG2 | THR | M | 69 | 20.743 | −18.338 | −27.133 | 1.00 | 24.17 | C |
| ATOM | 5560 | C | THR | M | 69 | 22.188 | −16.594 | −24.060 | 1.00 | 23.07 | C |
| ATOM | 5561 | O | THR | M | 69 | 23.400 | −16.646 | −24.295 | 1.00 | 23.07 | O |
| ATOM | 5562 | N | ASP | M | 70 | 21.648 | −15.727 | −23.203 | 1.00 | 23.17 | N |
| ATOM | 5563 | CA | ASP | M | 70 | 22.418 | −14.593 | −22.661 | 1.00 | 23.45 | C |
| ATOM | 5564 | CB | ASP | M | 70 | 21.897 | −13.278 | −23.247 | 1.00 | 24.51 | C |
| ATOM | 5565 | CG | ASP | M | 70 | 22.562 | −12.058 | −22.634 | 1.00 | 27.51 | C |
| ATOM | 5566 | OD1 | ASP | M | 70 | 23.755 | −11.814 | −22.927 | 1.00 | 30.37 | O |
| ATOM | 5567 | OD2 | ASP | M | 70 | 21.879 | −11.334 | −21.872 | 1.00 | 31.55 | O |
| ATOM | 5568 | C | ASP | M | 70 | 22.380 | −14.534 | −21.136 | 1.00 | 22.62 | C |
| ATOM | 5569 | O | ASP | M | 70 | 21.320 | −14.362 | −20.547 | 1.00 | 21.67 | O |
| ATOM | 5570 | N | PHE | M | 71 | 23.550 | −14.648 | −20.511 | 1.00 | 21.89 | N |
| ATOM | 5571 | CA | PHE | M | 71 | 23.610 | −14.761 | −19.059 | 1.00 | 21.41 | C |
| ATOM | 5572 | CB | PHE | M | 71 | 23.834 | −16.220 | −18.659 | 1.00 | 21.49 | C |
| ATOM | 5573 | CG | PHE | M | 71 | 22.852 | −17.167 | −19.286 | 1.00 | 20.04 | C |
| ATOM | 5574 | CD1 | PHE | M | 71 | 21.653 | −17.469 | −18.641 | 1.00 | 20.47 | C |
| ATOM | 5575 | CE1 | PHE | M | 71 | 20.721 | −18.333 | −19.225 | 1.00 | 20.30 | C |
| ATOM | 5576 | CZ | PHE | M | 71 | 20.984 | −18.892 | −20.487 | 1.00 | 20.88 | C |
| ATOM | 5577 | CE2 | PHE | M | 71 | 22.181 | −18.588 | −21.144 | 1.00 | 18.70 | C |
| ATOM | 5578 | CD2 | PHE | M | 71 | 23.104 | −17.718 | −20.542 | 1.00 | 20.64 | C |
| ATOM | 5579 | C | PHE | M | 71 | 24.677 | −13.871 | −18.456 | 1.00 | 21.58 | C |
| ATOM | 5580 | O | PHE | M | 71 | 25.655 | −13.507 | −19.122 | 1.00 | 20.78 | O |
| ATOM | 5581 | N | THR | M | 72 | 24.483 | −13.533 | −17.179 | 1.00 | 21.30 | N |
| ATOM | 5582 | CA | THR | M | 72 | 25.387 | −12.629 | −16.486 | 1.00 | 21.14 | C |
| ATOM | 5583 | CB | THR | M | 72 | 24.853 | −11.177 | −16.579 | 1.00 | 21.35 | C |
| ATOM | 5584 | OG1 | THR | M | 72 | 24.741 | −10.829 | −17.968 | 1.00 | 22.56 | O |
| ATOM | 5585 | CG2 | THR | M | 72 | 25.787 | −10.173 | −15.875 | 1.00 | 21.14 | C |
| ATOM | 5586 | C | THR | M | 72 | 25.589 | −13.060 | −15.042 | 1.00 | 20.79 | C |
| ATOM | 5587 | O | THR | M | 72 | 24.627 | −13.393 | −14.350 | 1.00 | 20.33 | O |
| ATOM | 5588 | N | LEU | M | 73 | 26.854 | −13.102 | −14.629 | 1.00 | 20.69 | N |
| ATOM | 5589 | CA | LEU | M | 73 | 27.224 | −13.272 | −13.230 | 1.00 | 21.02 | C |
| ATOM | 5590 | CB | LEU | M | 73 | 28.354 | −14.298 | −13.077 | 1.00 | 20.88 | C |
| ATOM | 5591 | CG | LEU | M | 73 | 29.009 | −14.531 | −11.705 | 1.00 | 22.16 | C |
| ATOM | 5592 | CD1 | LEU | M | 73 | 28.018 | −15.024 | −10.691 | 1.00 | 26.01 | C |
| ATOM | 5593 | CD2 | LEU | M | 73 | 30.116 | −15.551 | −11.809 | 1.00 | 24.22 | C |
| ATOM | 5594 | C | LEU | M | 73 | 27.673 | −11.907 | −12.706 | 1.00 | 21.24 | C |
| ATOM | 5595 | O | LEU | M | 73 | 28.456 | −11.203 | −13.362 | 1.00 | 20.84 | O |
| ATOM | 5596 | N | LYS | M | 74 | 27.173 | −11.543 | −11.530 | 1.00 | 21.02 | N |
| ATOM | 5597 | CA | LYS | M | 74 | 27.559 | −10.301 | −10.897 | 1.00 | 21.50 | C |
| ATOM | 5598 | CB | LYS | M | 74 | 26.379 | −9.356 | −10.819 | 1.00 | 22.10 | C |
| ATOM | 5599 | CG | LYS | M | 74 | 26.214 | −8.475 | −12.017 | 1.00 | 24.44 | C |
| ATOM | 5600 | CD | LYS | M | 74 | 25.115 | −7.463 | −11.750 | 1.00 | 29.24 | C |
| ATOM | 5601 | CE | LYS | M | 74 | 23.811 | −7.933 | −12.349 | 1.00 | 31.99 | C |
| ATOM | 5602 | NZ | LYS | M | 74 | 23.861 | −7.788 | −13.843 | 1.00 | 33.84 | N |
| ATOM | 5603 | C | LYS | M | 74 | 28.077 | −10.515 | −9.496 | 1.00 | 21.49 | C |
| ATOM | 5604 | O | LYS | M | 74 | 27.536 | −11.322 | −8.743 | 1.00 | 20.79 | O |
| ATOM | 5605 | N | ILE | M | 75 | 29.120 | −9.766 | −9.154 | 1.00 | 20.99 | N |
| ATOM | 5606 | CA | ILE | M | 75 | 29.559 | −9.663 | −7.771 | 1.00 | 21.40 | C |
| ATOM | 5607 | CB | ILE | M | 75 | 31.052 | −9.983 | −7.612 | 1.00 | 21.01 | C |
| ATOM | 5608 | CG1 | ILE | M | 75 | 31.384 | −11.282 | −8.328 | 1.00 | 22.54 | C |
| ATOM | 5609 | CD1 | ILE | M | 75 | 32.882 | −11.505 | −8.598 | 1.00 | 23.92 | C |
| ATOM | 5610 | CG2 | ILE | M | 75 | 31.419 | −10.031 | −6.122 | 1.00 | 20.20 | C |
| ATOM | 5611 | C | ILE | M | 75 | 29.303 | −8.220 | −7.336 | 1.00 | 21.79 | C |
| ATOM | 5612 | O | ILE | M | 75 | 29.788 | −7.283 | −7.979 | 1.00 | 21.07 | O |
| ATOM | 5613 | N | SER | M | 76 | 28.523 | −8.049 | −6.273 | 1.00 | 21.91 | N |
| ATOM | 5614 | CA | SER | M | 76 | 28.142 | −6.709 | −5.803 | 1.00 | 22.65 | C |
| ATOM | 5615 | CB | SER | M | 76 | 27.116 | −6.811 | −4.672 | 1.00 | 22.61 | C |
| ATOM | 5616 | OG | SER | M | 76 | 27.632 | −7.589 | −3.604 | 1.00 | 20.54 | O |
| ATOM | 5617 | C | SER | M | 76 | 29.354 | −5.910 | −5.322 | 1.00 | 23.78 | C |
| ATOM | 5618 | O | SER | M | 76 | 29.517 | −4.734 | −5.653 | 1.00 | 23.06 | O |
| ATOM | 5619 | N | ARG | M | 77 | 30.192 | −6.567 | −4.532 | 1.00 | 24.85 | N |
| ATOM | 5620 | CA | ARG | M | 77 | 31.399 | −5.964 | −3.965 | 1.00 | 26.24 | C |
| ATOM | 5621 | CB | ARG | M | 77 | 31.187 | −5.608 | −2.499 | 1.00 | 26.74 | C |
| ATOM | 5622 | CG | ARG | M | 77 | 30.356 | −4.398 | −2.173 | 1.00 | 29.73 | C |
| ATOM | 5623 | CD | ARG | M | 77 | 30.212 | −4.417 | −0.657 | 1.00 | 35.04 | C |
| ATOM | 5624 | NE | ARG | M | 77 | 29.745 | −3.178 | −0.042 | 1.00 | 39.37 | N |
| ATOM | 5625 | CZ | ARG | M | 77 | 30.539 | −2.196 | 0.385 | 1.00 | 40.79 | C |
| ATOM | 5626 | NH1 | ARG | M | 77 | 31.859 | −2.271 | 0.229 | 1.00 | 41.15 | N |
| ATOM | 5627 | NH2 | ARG | M | 77 | 30.003 | −1.127 | 0.962 | 1.00 | 41.47 | N |
| ATOM | 5628 | C | ARG | M | 77 | 32.457 | −7.043 | −3.997 | 1.00 | 26.03 | C |
| ATOM | 5629 | O | ARG | M | 77 | 32.313 | −8.058 | −3.312 | 1.00 | 25.98 | O |
| ATOM | 5630 | N | VAL | M | 78 | 33.505 | −6.836 | −4.784 | 1.00 | 25.98 | N |
| ATOM | 5631 | CA | VAL | M | 78 | 34.534 | −7.852 | −4.965 | 1.00 | 26.60 | C |
| ATOM | 5632 | CB | VAL | M | 78 | 35.317 | −7.653 | −6.288 | 1.00 | 26.38 | C |
| ATOM | 5633 | CG1 | VAL | M | 78 | 36.626 | −8.419 | −6.269 | 1.00 | 26.35 | C |
| ATOM | 5634 | CG2 | VAL | M | 78 | 34.485 | −8.107 | −7.469 | 1.00 | 25.21 | C |
| ATOM | 5635 | C | VAL | M | 78 | 35.491 | −7.900 | −3.779 | 1.00 | 27.31 | C |
| ATOM | 5636 | O | VAL | M | 78 | 35.917 | −6.858 | −3.269 | 1.00 | 27.56 | O |
| ATOM | 5637 | N | GLU | M | 79 | 35.822 | −9.114 | −3.346 | 1.00 | 27.71 | N |
| ATOM | 5638 | CA | GLU | M | 79 | 36.812 | −9.296 | −2.289 | 1.00 | 28.45 | C |
| ATOM | 5639 | CB | GLU | M | 79 | 36.165 | −9.982 | −1.106 | 1.00 | 29.14 | C |
| ATOM | 5640 | CG | GLU | M | 79 | 35.107 | −9.083 | −0.514 | 1.00 | 34.02 | C |
| ATOM | 5641 | CD | GLU | M | 79 | 34.475 | −9.648 | 0.704 | 1.00 | 38.13 | C |
| ATOM | 5642 | OE1 | GLU | M | 79 | 34.269 | −10.879 | 0.727 | 1.00 | 40.49 | O |
| ATOM | 5643 | OE2 | GLU | M | 79 | 34.186 | −8.856 | 1.633 | 1.00 | 40.75 | O |
| ATOM | 5644 | C | GLU | M | 79 | 38.049 | −10.039 | −2.780 | 1.00 | 27.82 | C |
| ATOM | 5645 | O | GLU | M | 79 | 38.013 | −10.696 | −3.821 | 1.00 | 26.92 | O |
| ATOM | 5646 | N | ALA | M | 80 | 39.155 | −9.910 | −2.046 | 1.00 | 27.22 | N |
| ATOM | 5647 | CA | ALA | M | 80 | 40.419 | −10.519 | −2.467 | 1.00 | 26.66 | C |
| ATOM | 5648 | CB | ALA | M | 80 | 41.507 | −10.308 | −1.407 | 1.00 | 27.16 | C |
| ATOM | 5649 | C | ALA | M | 80 | 40.262 | −12.011 | −2.799 | 1.00 | 26.11 | C |
| ATOM | 5650 | O | ALA | M | 80 | 40.914 | −12.511 | −3.726 | 1.00 | 26.21 | O |
| ATOM | 5651 | N | GLU | M | 81 | 39.367 | −12.688 | −2.073 | 1.00 | 24.71 | N |
| ATOM | 5652 | CA | GLU | M | 81 | 39.099 | −14.122 | −2.245 | 1.00 | 24.42 | C |
| ATOM | 5653 | CB | GLU | M | 81 | 38.221 | −14.662 | −1.103 | 1.00 | 24.83 | C |
| ATOM | 5654 | CG | GLU | M | 81 | 38.649 | −14.273 | 0.291 | 1.00 | 29.87 | C |
| ATOM | 5655 | CD | GLU | M | 81 | 38.010 | −12.962 | 0.758 | 1.00 | 33.58 | C |
| ATOM | 5656 | OE1 | GLU | M | 81 | 36.797 | −12.970 | 1.114 | 1.00 | 36.95 | O |
| ATOM | 5657 | OE2 | GLU | M | 81 | 38.725 | −11.933 | 0.762 | 1.00 | 33.23 | O |
| ATOM | 5658 | C | GLU | M | 81 | 38.396 | −14.467 | −3.548 | 1.00 | 23.13 | C |
| ATOM | 5659 | O | GLU | M | 81 | 38.217 | −15.642 | −3.856 | 1.00 | 21.76 | O |
| ATOM | 5660 | N | ASP | M | 82 | 37.975 | −13.455 | −4.299 | 1.00 | 21.83 | N |
| ATOM | 5661 | CA | ASP | M | 82 | 37.135 | −13.690 | −5.468 | 1.00 | 21.33 | C |
| ATOM | 5662 | CB | ASP | M | 82 | 36.051 | −12.603 | −5.614 | 1.00 | 21.23 | C |
| ATOM | 5663 | CG | ASP | M | 82 | 35.086 | −12.570 | −4.443 | 1.00 | 21.86 | C |
| ATOM | 5664 | OD1 | ASP | M | 82 | 34.942 | −13.593 | −3.737 | 1.00 | 20.97 | O |
| ATOM | 5665 | OD2 | ASP | M | 82 | 34.444 | −11.515 | −4.229 | 1.00 | 24.60 | O |
| ATOM | 5666 | C | ASP | M | 82 | 37.969 | −13.815 | −6.739 | 1.00 | 21.05 | C |
| ATOM | 5667 | O | ASP | M | 82 | 37.424 | −14.042 | −7.825 | 1.00 | 20.04 | O |
| ATOM | 5668 | N | VAL | M | 83 | 39.288 | −13.672 | −6.590 | 1.00 | 20.43 | N |
| ATOM | 5669 | CA | VAL | M | 83 | 40.205 | −13.801 | −7.706 | 1.00 | 20.33 | C |
| ATOM | 5670 | CB | VAL | M | 83 | 41.578 | −13.225 | −7.346 | 1.00 | 21.20 | C |
| ATOM | 5671 | CG1 | VAL | M | 83 | 42.553 | −13.534 | −8.435 | 1.00 | 22.74 | C |
| ATOM | 5672 | CG2 | VAL | M | 83 | 41.459 | −11.693 | −7.149 | 1.00 | 19.94 | C |
| ATOM | 5673 | C | VAL | M | 83 | 40.344 | −15.242 | −8.213 | 1.00 | 20.20 | C |
| ATOM | 5674 | O | VAL | M | 83 | 40.337 | −16.204 | −7.429 | 1.00 | 18.93 | O |
| ATOM | 5675 | N | GLY | M | 84 | 40.470 | −15.377 | −9.537 | 1.00 | 19.70 | N |
| ATOM | 5676 | CA | GLY | M | 84 | 40.614 | −16.678 | −10.177 | 1.00 | 19.85 | C |
| ATOM | 5677 | C | GLY | M | 84 | 39.911 | −16.659 | −11.516 | 1.00 | 19.56 | C |
| ATOM | 5678 | O | GLY | M | 84 | 39.601 | −15.585 | −12.035 | 1.00 | 19.39 | O |
| ATOM | 5679 | N | VAL | M | 85 | 39.649 | −17.831 | −12.078 | 1.00 | 18.59 | N |
| ATOM | 5680 | CA | VAL | M | 85 | 39.012 | −17.891 | −13.381 | 1.00 | 19.41 | C |
| ATOM | 5681 | CB | VAL | M | 85 | 39.788 | −18.794 | −14.397 | 1.00 | 19.21 | C |
| ATOM | 5682 | CG1 | VAL | M | 85 | 39.107 | −18.771 | −15.761 | 1.00 | 20.41 | C |
| ATOM | 5683 | CG2 | VAL | M | 85 | 41.256 | −18.339 | −14.526 | 1.00 | 20.92 | C |
| ATOM | 5684 | C | VAL | M | 85 | 37.567 | −18.341 | −13.253 | 1.00 | 19.01 | C |
| ATOM | 5685 | O | VAL | M | 85 | 37.288 | −19.437 | −12.735 | 1.00 | 18.96 | O |
| ATOM | 5686 | N | TYR | M | 86 | 36.669 | −17.491 | −13.739 | 1.00 | 18.61 | N |
| ATOM | 5687 | CA | TYR | M | 86 | 35.236 | −17.782 | −13.760 | 1.00 | 19.05 | C |
| ATOM | 5688 | CB | TYR | M | 86 | 34.436 | −16.479 | −13.559 | 1.00 | 18.12 | C |
| ATOM | 5689 | CG | TYR | M | 86 | 34.565 | −15.942 | −12.152 | 1.00 | 19.31 | C |
| ATOM | 5690 | CD1 | TYR | M | 86 | 35.675 | −15.159 | −11.766 | 1.00 | 17.61 | C |
| ATOM | 5691 | CE1 | TYR | M | 86 | 35.804 | −14.687 | −10.470 | 1.00 | 18.69 | C |
| ATOM | 5692 | CZ | TYR | M | 86 | 34.805 | −14.995 | −9.536 | 1.00 | 18.46 | C |
| ATOM | 5693 | OH | TYR | M | 86 | 34.899 | −14.565 | −8.242 | 1.00 | 17.53 | O |
| ATOM | 5694 | CE2 | TYR | M | 86 | 33.716 | −15.769 | −9.895 | 1.00 | 18.94 | C |
| ATOM | 5695 | CD2 | TYR | M | 86 | 33.599 | −16.239 | −11.186 | 1.00 | 18.89 | C |
| ATOM | 5696 | C | TYR | M | 86 | 34.830 | −18.474 | −15.057 | 1.00 | 18.55 | C |
| ATOM | 5697 | O | TYR | M | 86 | 35.137 | −17.990 | −16.143 | 1.00 | 19.09 | O |
| ATOM | 5698 | N | PHE | M | 87 | 34.135 | −19.604 | −14.944 | 1.00 | 18.42 | N |
| ATOM | 5699 | CA | PHE | M | 87 | 33.658 | −20.338 | −16.118 | 1.00 | 18.53 | C |
| ATOM | 5700 | CB | PHE | M | 87 | 34.228 | −21.757 | −16.148 | 1.00 | 19.05 | C |
| ATOM | 5701 | CG | PHE | M | 87 | 35.710 | −21.837 | −16.369 | 1.00 | 17.84 | C |
| ATOM | 5702 | CD1 | PHE | M | 87 | 36.245 | −21.680 | −17.642 | 1.00 | 18.58 | C |
| ATOM | 5703 | CE1 | PHE | M | 87 | 37.609 | −21.771 | −17.865 | 1.00 | 17.96 | C |
| ATOM | 5704 | CZ | PHE | M | 87 | 38.467 | −22.049 | −16.795 | 1.00 | 18.91 | C |
| ATOM | 5705 | CE2 | PHE | M | 87 | 37.930 | −22.225 | −15.508 | 1.00 | 19.92 | C |
| ATOM | 5706 | CD2 | PHE | M | 87 | 36.553 | −22.112 | −15.315 | 1.00 | 18.56 | C |
| ATOM | 5707 | C | PHE | M | 87 | 32.152 | −20.478 | −16.084 | 1.00 | 18.67 | C |
| ATOM | 5708 | O | PHE | M | 87 | 31.589 | −20.846 | −15.042 | 1.00 | 19.11 | O |
| ATOM | 5709 | N | CYS | M | 88 | 31.494 | −20.211 | −17.208 | 1.00 | 18.49 | N |
| ATOM | 5710 | CA | CYS | M | 88 | 30.104 | −20.639 | −17.359 | 1.00 | 19.17 | C |
| ATOM | 5711 | CB | CYS | M | 88 | 29.285 | −19.692 | −18.214 | 1.00 | 18.89 | C |
| ATOM | 5712 | SG | CYS | M | 88 | 29.867 | −19.468 | −19.913 | 1.00 | 22.34 | S |
| ATOM | 5713 | C | CYS | M | 88 | 30.042 | −22.085 | −17.869 | 1.00 | 18.49 | C |
| ATOM | 5714 | O | CYS | M | 88 | 30.958 | −22.559 | −18.536 | 1.00 | 19.20 | O |
| ATOM | 5715 | N | SER | M | 89 | 28.969 | −22.787 | −17.525 | 1.00 | 18.64 | N |
| ATOM | 5716 | CA | SER | M | 89 | 28.865 | −24.223 | −17.790 | 1.00 | 17.81 | C |
| ATOM | 5717 | CB | SER | M | 89 | 29.249 | −25.003 | −16.520 | 1.00 | 17.51 | C |
| ATOM | 5718 | OG | SER | M | 89 | 28.812 | −26.366 | −16.550 | 1.00 | 18.71 | O |
| ATOM | 5719 | C | SER | M | 89 | 27.437 | −24.519 | −18.196 | 1.00 | 18.14 | C |
| ATOM | 5720 | O | SER | M | 89 | 26.523 | −23.997 | −17.574 | 1.00 | 19.33 | O |
| ATOM | 5721 | N | GLN | M | 90 | 27.231 | −25.339 | −19.227 | 1.00 | 17.98 | N |
| ATOM | 5722 | CA | GLN | M | 90 | 25.867 | −25.745 | −19.611 | 1.00 | 17.84 | C |
| ATOM | 5723 | CB | GLN | M | 90 | 25.472 | −25.216 | −21.001 | 1.00 | 18.02 | C |
| ATOM | 5724 | CG | GLN | M | 90 | 26.172 | −25.876 | −22.198 | 1.00 | 19.05 | C |
| ATOM | 5725 | CD | GLN | M | 90 | 25.486 | −27.125 | −22.763 | 1.00 | 18.44 | C |
| ATOM | 5726 | OE1 | GLN | M | 90 | 24.348 | −27.461 | −22.405 | 1.00 | 19.18 | O |
| ATOM | 5727 | NE2 | GLN | M | 90 | 26.193 | −27.825 | −23.657 | 1.00 | 18.09 | N |
| ATOM | 5728 | C | GLN | M | 90 | 25.701 | −27.259 | −19.557 | 1.00 | 17.73 | C |
| ATOM | 5729 | O | GLN | M | 90 | 26.596 | −28.012 | −19.976 | 1.00 | 17.65 | O |
| ATOM | 5730 | N | SER | M | 91 | 24.562 | −27.699 | −19.035 | 1.00 | 17.18 | N |
| ATOM | 5731 | CA | SER | M | 91 | 24.247 | −29.115 | −19.003 | 1.00 | 16.84 | C |
| ATOM | 5732 | CB | SER | M | 91 | 24.398 | −29.699 | −17.593 | 1.00 | 16.55 | C |
| ATOM | 5733 | OG | SER | M | 91 | 23.479 | −29.108 | −16.682 | 1.00 | 17.16 | O |
| ATOM | 5734 | C | SER | M | 91 | 22.871 | −29.416 | −19.607 | 1.00 | 17.14 | C |
| ATOM | 5735 | O | SER | M | 91 | 22.233 | −30.423 | −19.287 | 1.00 | 16.42 | O |
| ATOM | 5736 | N | THR | M | 92 | 22.437 | −28.544 | −20.505 | 1.00 | 17.85 | N |
| ATOM | 5737 | CA | THR | M | 92 | 21.209 | −28.753 | −21.260 | 1.00 | 18.68 | C |
| ATOM | 5738 | CB | THR | M | 92 | 20.681 | −27.430 | −21.815 | 1.00 | 19.07 | C |
| ATOM | 5739 | OG1 | THR | M | 92 | 20.475 | −26.524 | −20.725 | 1.00 | 19.78 | O |
| ATOM | 5740 | CG2 | THR | M | 92 | 19.353 | −27.636 | −22.556 | 1.00 | 18.36 | C |
| ATOM | 5741 | C | THR | M | 92 | 21.389 | −29.754 | −22.396 | 1.00 | 19.15 | C |
| ATOM | 5742 | O | THR | M | 92 | 20.571 | −30.662 | −22.546 | 1.00 | 19.44 | O |
| ATOM | 5743 | N | HIS | M | 93 | 22.452 | −29.579 | −23.184 | 1.00 | 19.50 | N |
| ATOM | 5744 | CA | HIS | M | 93 | 22.699 | −30.380 | −24.385 | 1.00 | 19.74 | C |
| ATOM | 5745 | CB | HIS | M | 93 | 22.785 | −29.468 | −25.607 | 1.00 | 20.84 | C |
| ATOM | 5746 | CG | HIS | M | 93 | 21.620 | −28.541 | −25.766 | 1.00 | 23.31 | C |
| ATOM | 5747 | ND1 | HIS | M | 93 | 20.367 | −28.980 | −26.141 | 1.00 | 24.69 | N |
| ATOM | 5748 | CE1 | HIS | M | 93 | 19.545 | −27.947 | −26.211 | 1.00 | 25.80 | C |
| ATOM | 5749 | NE2 | HIS | M | 93 | 20.222 | −26.853 | −25.902 | 1.00 | 26.16 | N |
| ATOM | 5750 | CD2 | HIS | M | 93 | 21.524 | −27.196 | −25.623 | 1.00 | 25.05 | C |
| ATOM | 5751 | C | HIS | M | 93 | 24.034 | −31.103 | −24.296 | 1.00 | 19.12 | C |
| ATOM | 5752 | O | HIS | M | 93 | 25.036 | −30.520 | −23.873 | 1.00 | 18.41 | O |
| ATOM | 5753 | N | PHE | M | 94 | 24.056 | −32.365 | −24.709 | 1.00 | 17.86 | N |
| ATOM | 5754 | CA | PHE | M | 94 | 25.322 | −33.060 | −24.929 | 1.00 | 17.67 | C |
| ATOM | 5755 | CB | PHE | M | 94 | 25.124 | −34.584 | −24.915 | 1.00 | 18.11 | C |
| ATOM | 5756 | CG | PHE | M | 94 | 24.895 | −35.144 | −23.546 | 1.00 | 18.70 | C |
| ATOM | 5757 | CD1 | PHE | M | 94 | 25.915 | −35.129 | −22.598 | 1.00 | 18.90 | C |
| ATOM | 5758 | CE1 | PHE | M | 94 | 25.716 | −35.636 | −21.331 | 1.00 | 20.26 | C |
| ATOM | 5759 | CZ | PHE | M | 94 | 24.479 | −36.190 | −20.994 | 1.00 | 22.15 | C |
| ATOM | 5760 | CE2 | PHE | M | 94 | 23.453 | −36.221 | −21.936 | 1.00 | 21.62 | C |
| ATOM | 5761 | CD2 | PHE | M | 94 | 23.668 | −35.701 | −23.209 | 1.00 | 21.18 | C |
| ATOM | 5762 | C | PHE | M | 94 | 25.912 | −32.637 | −26.267 | 1.00 | 17.22 | C |
| ATOM | 5763 | O | PHE | M | 94 | 25.165 | −32.402 | −27.212 | 1.00 | 17.17 | O |
| ATOM | 5764 | N | PRO | M | 95 | 27.253 | −32.529 | −26.353 | 1.00 | 17.34 | N |
| ATOM | 5765 | CA | PRO | M | 95 | 28.208 | −32.660 | −25.237 | 1.00 | 16.67 | C |
| ATOM | 5766 | CB | PRO | M | 95 | 29.570 | −32.720 | −25.950 | 1.00 | 16.96 | C |
| ATOM | 5767 | CG | PRO | M | 95 | 29.343 | −32.017 | −27.265 | 1.00 | 17.46 | C |
| ATOM | 5768 | CD | PRO | M | 95 | 27.948 | −32.480 | −27.656 | 1.00 | 16.87 | C |
| ATOM | 5769 | C | PRO | M | 95 | 28.169 | −31.448 | −24.306 | 1.00 | 17.12 | C |
| ATOM | 5770 | O | PRO | M | 95 | 28.042 | −30.320 | −24.780 | 1.00 | 17.05 | O |
| ATOM | 5771 | N | PHE | M | 96 | 28.286 | −31.671 | −22.994 | 1.00 | 16.74 | N |
| ATOM | 5772 | CA | PHE | M | 96 | 28.353 | −30.553 | −22.050 | 1.00 | 16.57 | C |
| ATOM | 5773 | CB | PHE | M | 96 | 28.435 | −31.027 | −20.593 | 1.00 | 16.01 | C |
| ATOM | 5774 | CG | PHE | M | 96 | 27.205 | −31.778 | −20.108 | 1.00 | 17.57 | C |
| ATOM | 5775 | CD1 | PHE | M | 96 | 27.239 | −32.473 | −18.905 | 1.00 | 17.69 | C |
| ATOM | 5776 | CE1 | PHE | M | 96 | 26.132 | −33.168 | −18.446 | 1.00 | 17.12 | C |
| ATOM | 5777 | CZ | PHE | M | 96 | 24.959 | −33.175 | −19.197 | 1.00 | 19.89 | C |
| ATOM | 5778 | CE2 | PHE | M | 96 | 24.913 | −32.486 | −20.412 | 1.00 | 19.73 | C |
| ATOM | 5779 | CD2 | PHE | M | 96 | 26.027 | −31.790 | −20.849 | 1.00 | 17.48 | C |
| ATOM | 5780 | C | PHE | M | 96 | 29.572 | −29.701 | −22.415 | 1.00 | 16.54 | C |
| ATOM | 5781 | O | PHE | M | 96 | 30.583 | −30.222 | −22.903 | 1.00 | 16.36 | O |
| ATOM | 5782 | N | THR | M | 97 | 29.472 | −28.394 | −22.202 | 1.00 | 16.76 | N |
| ATOM | 5783 | CA | THR | M | 97 | 30.521 | −27.474 | −22.646 | 1.00 | 16.69 | C |
| ATOM | 5784 | CB | THR | M | 97 | 30.172 | −26.806 | −23.992 | 1.00 | 16.59 | C |
| ATOM | 5785 | OG1 | THR | M | 97 | 28.880 | −26.214 | −23.881 | 1.00 | 16.87 | O |
| ATOM | 5786 | CG2 | THR | M | 97 | 30.207 | −27.794 | −25.158 | 1.00 | 16.42 | C |
| ATOM | 5787 | C | THR | M | 97 | 30.720 | −26.377 | −21.616 | 1.00 | 16.71 | C |
| ATOM | 5788 | O | THR | M | 97 | 29.863 | −26.177 | −20.746 | 1.00 | 16.80 | O |
| ATOM | 5789 | N | PHE | M | 98 | 31.863 | −25.697 | −21.713 | 1.00 | 16.99 | N |
| ATOM | 5790 | CA | PHE | M | 98 | 32.252 | −24.600 | −20.825 | 1.00 | 17.73 | C |
| ATOM | 5791 | CB | PHE | M | 98 | 33.407 | −25.032 | −19.913 | 1.00 | 17.88 | C |
| ATOM | 5792 | CG | PHE | M | 98 | 33.054 | −26.154 | −18.971 | 1.00 | 18.64 | C |
| ATOM | 5793 | CD1 | PHE | M | 98 | 33.240 | −27.480 | −19.343 | 1.00 | 18.21 | C |
| ATOM | 5794 | CE1 | PHE | M | 98 | 32.908 | −28.530 | −18.463 | 1.00 | 19.34 | C |
| ATOM | 5795 | CZ | PHE | M | 98 | 32.398 | −28.229 | −17.202 | 1.00 | 17.81 | C |
| ATOM | 5796 | CE2 | PHE | M | 98 | 32.223 | −26.906 | −16.830 | 1.00 | 18.10 | C |
| ATOM | 5797 | CD2 | PHE | M | 98 | 32.552 | −25.878 | −17.710 | 1.00 | 18.86 | C |
| ATOM | 5798 | C | PHE | M | 98 | 32.712 | −23.391 | −21.636 | 1.00 | 18.62 | C |
| ATOM | 5799 | O | PHE | M | 98 | 33.237 | −23.540 | −22.747 | 1.00 | 18.26 | O |
| ATOM | 5800 | N | GLY | M | 99 | 32.530 | −22.199 | −21.069 | 1.00 | 19.74 | N |
| ATOM | 5801 | CA | GLY | M | 99 | 33.095 | −20.977 | −21.622 | 1.00 | 20.66 | C |
| ATOM | 5802 | C | GLY | M | 99 | 34.612 | −21.013 | −21.518 | 1.00 | 22.44 | C |
| ATOM | 5803 | O | GLY | M | 99 | 35.176 | −21.844 | −20.800 | 1.00 | 22.17 | O |
| ATOM | 5804 | N | GLN | M | 100 | 35.286 | −20.120 | −22.239 | 1.00 | 23.46 | N |
| ATOM | 5805 | CA | GLN | M | 100 | 36.755 | −20.092 | −22.220 | 1.00 | 24.69 | C |
| ATOM | 5806 | CB | GLN | M | 100 | 37.330 | −19.293 | −23.406 | 1.00 | 25.75 | C |
| ATOM | 5807 | CG | GLN | M | 100 | 36.692 | −17.907 | −23.621 | 1.00 | 28.00 | C |
| ATOM | 5808 | CD | GLN | M | 100 | 35.463 | −17.967 | −24.530 | 1.00 | 31.70 | C |
| ATOM | 5809 | OE1 | GLN | M | 100 | 34.344 | −18.192 | −24.056 | 1.00 | 30.89 | O |
| ATOM | 5810 | NE2 | GLN | M | 100 | 35.672 | −17.772 | −25.845 | 1.00 | 30.44 | N |
| ATOM | 5811 | C | GLN | M | 100 | 37.310 | −19.577 | −20.895 | 1.00 | 23.89 | C |
| ATOM | 5812 | O | GLN | M | 100 | 38.468 | −19.794 | −20.583 | 1.00 | 24.40 | O |
| ATOM | 5813 | N | GLY | M | 101 | 36.478 | −18.906 | −20.112 | 1.00 | 23.86 | N |
| ATOM | 5814 | CA | GLY | M | 101 | 36.890 | −18.445 | −18.794 | 1.00 | 23.28 | C |
| ATOM | 5815 | C | GLY | M | 101 | 37.159 | −16.943 | −18.729 | 1.00 | 23.10 | C |
| ATOM | 5816 | O | GLY | M | 101 | 37.608 | −16.348 | −19.707 | 1.00 | 23.34 | O |
| ATOM | 5817 | N | THR | M | 102 | 36.880 | −16.342 | −17.572 | 1.00 | 22.17 | N |
| ATOM | 5818 | CA | THR | M | 102 | 37.174 | −14.919 | −17.321 | 1.00 | 21.93 | C |
| ATOM | 5819 | CB | THR | M | 102 | 35.899 | −14.115 | −17.042 | 1.00 | 21.55 | C |
| ATOM | 5820 | OG1 | THR | M | 102 | 35.121 | −14.081 | −18.229 | 1.00 | 21.45 | O |
| ATOM | 5821 | CG2 | THR | M | 102 | 36.210 | −12.642 | −16.610 | 1.00 | 21.70 | C |
| ATOM | 5822 | C | THR | M | 102 | 38.092 | −14.817 | −16.123 | 1.00 | 21.88 | C |
| ATOM | 5823 | O | THR | M | 102 | 37.723 | −15.237 | −15.028 | 1.00 | 21.14 | O |
| ATOM | 5824 | N | LYS | M | 103 | 39.290 | −14.272 | −16.340 | 1.00 | 21.59 | N |
| ATOM | 5825 | CA | LYS | M | 103 | 40.277 | −14.192 | −15.275 | 1.00 | 21.47 | C |
| ATOM | 5826 | CB | LYS | M | 103 | 41.711 | −14.393 | −15.786 | 1.00 | 22.28 | C |
| ATOM | 5827 | CG | LYS | M | 103 | 42.707 | −14.609 | −14.627 | 1.00 | 24.15 | C |
| ATOM | 5828 | CD | LYS | M | 103 | 44.172 | −14.417 | −15.055 | 1.00 | 29.64 | C |
| ATOM | 5829 | CE | LYS | M | 103 | 44.829 | −15.690 | −15.492 | 1.00 | 30.38 | C |
| ATOM | 5830 | NZ | LYS | M | 103 | 46.238 | −15.736 | −14.985 | 1.00 | 30.81 | N |
| ATOM | 5831 | C | LYS | M | 103 | 40.150 | −12.899 | −14.495 | 1.00 | 21.30 | C |
| ATOM | 5832 | O | LYS | M | 103 | 40.423 | −11.787 | −14.993 | 1.00 | 19.57 | O |
| ATOM | 5833 | N | LEU | M | 104 | 39.714 | −13.040 | −13.253 | 1.00 | 21.12 | N |
| ATOM | 5834 | CA | LEU | M | 104 | 39.519 | −11.881 | −12.409 | 1.00 | 21.53 | C |
| ATOM | 5835 | CB | LEU | M | 104 | 38.316 | −12.090 | −11.499 | 1.00 | 22.60 | C |
| ATOM | 5836 | CG | LEU | M | 104 | 37.736 | −10.830 | −10.860 | 1.00 | 23.67 | C |
| ATOM | 5837 | CD1 | LEU | M | 104 | 36.246 | −10.989 | −10.696 | 1.00 | 25.36 | C |
| ATOM | 5838 | CD2 | LEU | M | 104 | 38.422 | −10.538 | −9.517 | 1.00 | 25.47 | C |
| ATOM | 5839 | C | LEU | M | 104 | 40.789 | −11.697 | −11.603 | 1.00 | 21.59 | C |
| ATOM | 5840 | O | LEU | M | 104 | 41.285 | −12.661 | −10.999 | 1.00 | 20.52 | O |
| ATOM | 5841 | N | GLU | M | 105 | 41.328 | −10.475 | −11.638 | 1.00 | 21.03 | N |
| ATOM | 5842 | CA | GLU | M | 105 | 42.569 | −10.142 | −10.940 | 1.00 | 21.90 | C |
| ATOM | 5843 | CB | GLU | M | 105 | 43.753 | −10.098 | −11.932 | 1.00 | 22.97 | C |
| ATOM | 5844 | CG | GLU | M | 105 | 44.079 | −11.491 | −12.564 | 1.00 | 25.09 | C |
| ATOM | 5845 | CD | GLU | M | 105 | 45.298 | −11.486 | −13.493 | 1.00 | 29.54 | C |
| ATOM | 5846 | OE1 | GLU | M | 105 | 45.133 | −11.139 | −14.685 | 1.00 | 30.30 | O |
| ATOM | 5847 | OE2 | GLU | M | 105 | 46.414 | −11.855 | −13.038 | 1.00 | 29.42 | O |
| ATOM | 5848 | C | GLU | M | 105 | 42.401 | −8.809 | −10.203 | 1.00 | 21.58 | C |
| ATOM | 5849 | O | GLU | M | 105 | 41.369 | −8.153 | −10.345 | 1.00 | 21.68 | O |
| ATOM | 5850 | N | ILE | M | 106 | 43.391 | −8.424 | −9.404 | 1.00 | 20.78 | N |
| ATOM | 5851 | CA | ILE | M | 106 | 43.291 | −7.198 | −8.613 | 1.00 | 20.20 | C |
| ATOM | 5852 | CB | ILE | M | 106 | 43.989 | −7.336 | −7.225 | 1.00 | 20.62 | C |
| ATOM | 5853 | CG1 | ILE | M | 106 | 43.396 | −8.504 | −6.417 | 1.00 | 20.16 | C |
| ATOM | 5854 | CD1 | ILE | M | 106 | 41.926 | −8.329 | −6.054 | 1.00 | 21.83 | C |
| ATOM | 5855 | CG2 | ILE | M | 106 | 43.918 | −6.005 | −6.417 | 1.00 | 19.58 | C |
| ATOM | 5856 | C | ILE | M | 106 | 43.862 | −6.010 | −9.398 | 1.00 | 19.59 | C |
| ATOM | 5857 | O | ILE | M | 106 | 44.968 | −6.082 | −9.901 | 1.00 | 19.34 | O |
| ATOM | 5858 | N | LYS | M | 107 | 43.070 | −4.953 | −9.538 | 1.00 | 19.11 | N |
| ATOM | 5859 | CA | LYS | M | 107 | 43.515 | −3.715 | −10.183 | 1.00 | 19.33 | C |
| ATOM | 5860 | CB | LYS | M | 107 | 42.307 | −2.897 | −10.655 | 1.00 | 19.79 | C |
| ATOM | 5861 | CG | LYS | M | 107 | 42.686 | −1.629 | −11.433 | 1.00 | 23.30 | C |
| ATOM | 5862 | CD | LYS | M | 107 | 41.461 | −0.839 | −11.858 | 1.00 | 28.24 | C |
| ATOM | 5863 | CE | LYS | M | 107 | 41.867 | 0.549 | −12.338 | 1.00 | 32.55 | C |
| ATOM | 5864 | NZ | LYS | M | 107 | 42.606 | 0.506 | −13.637 | 1.00 | 35.14 | N |
| ATOM | 5865 | C | LYS | M | 107 | 44.298 | −2.916 | −9.162 | 1.00 | 18.34 | C |
| ATOM | 5866 | O | LYS | M | 107 | 43.928 | −2.886 | −7.983 | 1.00 | 18.25 | O |
| ATOM | 5867 | N | ARG | M | 108 | 45.384 | −2.286 | −9.611 | 1.00 | 17.23 | N |
| ATOM | 5868 | CA | ARG | M | 108 | 46.186 | −1.401 | −8.778 | 1.00 | 16.11 | C |
| ATOM | 5869 | CB | ARG | M | 108 | 47.245 | −2.191 | −7.994 | 1.00 | 16.48 | C |
| ATOM | 5870 | CG | ARG | M | 108 | 48.170 | −3.017 | −8.873 | 1.00 | 14.74 | C |
| ATOM | 5871 | CD | ARG | M | 108 | 49.583 | −3.096 | −8.304 | 1.00 | 15.63 | C |
| ATOM | 5872 | NE | ARG | M | 108 | 50.208 | −1.779 | −8.237 | 1.00 | 15.49 | N |
| ATOM | 5873 | CZ | ARG | M | 108 | 51.204 | −1.463 | −7.418 | 1.00 | 16.53 | C |
| ATOM | 5874 | NH1 | ARG | M | 108 | 51.690 | −0.236 | −7.427 | 1.00 | 16.60 | N |
| ATOM | 5875 | NH2 | ARG | M | 108 | 51.712 | −2.370 | −6.583 | 1.00 | 15.69 | N |
| ATOM | 5876 | C | ARG | M | 108 | 46.848 | −0.340 | −9.658 | 1.00 | 15.70 | C |
| ATOM | 5877 | O | ARG | M | 108 | 46.672 | −0.321 | −10.874 | 1.00 | 15.55 | O |
| ATOM | 5878 | N | THR | M | 109 | 47.614 | 0.544 | −9.048 | 1.00 | 15.54 | N |
| ATOM | 5879 | CA | THR | M | 109 | 48.268 | 1.610 | −9.811 | 1.00 | 14.78 | C |
| ATOM | 5880 | CB | THR | M | 109 | 48.859 | 2.657 | −8.874 | 1.00 | 15.22 | C |
| ATOM | 5881 | OG1 | THR | M | 109 | 49.758 | 2.009 | −7.966 | 1.00 | 13.92 | O |
| ATOM | 5882 | CG2 | THR | M | 109 | 47.751 | 3.355 | −8.092 | 1.00 | 15.04 | C |
| ATOM | 5883 | C | THR | M | 109 | 49.393 | 1.050 | −10.673 | 1.00 | 15.28 | C |
| ATOM | 5884 | O | THR | M | 109 | 49.943 | −0.044 | −10.395 | 1.00 | 14.65 | O |
| ATOM | 5885 | N | VAL | M | 110 | 49.750 | 1.790 | −11.718 | 1.00 | 14.75 | N |
| ATOM | 5886 | CA | VAL | M | 110 | 50.848 | 1.386 | −12.595 | 1.00 | 15.02 | C |
| ATOM | 5887 | CB | VAL | M | 110 | 50.937 | 2.295 | −13.840 | 1.00 | 15.92 | C |
| ATOM | 5888 | CG1 | VAL | M | 110 | 52.244 | 2.065 | −14.609 | 1.00 | 15.35 | C |
| ATOM | 5889 | CG2 | VAL | M | 110 | 49.736 | 2.053 | −14.731 | 1.00 | 15.43 | C |
| ATOM | 5890 | C | VAL | M | 110 | 52.165 | 1.385 | −11.832 | 1.00 | 15.33 | C |
| ATOM | 5891 | O | VAL | M | 110 | 52.463 | 2.313 | −11.084 | 1.00 | 15.92 | O |
| ATOM | 5892 | N | ALA | M | 111 | 52.945 | 0.335 | −12.025 | 1.00 | 15.45 | N |
| ATOM | 5893 | CA | ALA | M | 111 | 54.249 | 0.248 | −11.420 | 1.00 | 16.13 | C |
| ATOM | 5894 | CB | ALA | M | 111 | 54.209 | −0.654 | −10.203 | 1.00 | 16.12 | C |
| ATOM | 5895 | C | ALA | M | 111 | 55.281 | −0.252 | −12.424 | 1.00 | 16.46 | C |
| ATOM | 5896 | O | ALA | M | 111 | 55.072 | −1.268 | −13.099 | 1.00 | 16.29 | O |
| ATOM | 5897 | N | ALA | M | 112 | 56.404 | 0.460 | −12.488 | 1.00 | 16.80 | N |
| ATOM | 5898 | CA | ALA | M | 112 | 57.510 | 0.114 | −13.372 | 1.00 | 17.13 | C |
| ATOM | 5899 | CB | ALA | M | 112 | 58.475 | 1.307 | −13.490 | 1.00 | 17.47 | C |
| ATOM | 5900 | C | ALA | M | 112 | 58.261 | −1.118 | −12.880 | 1.00 | 17.75 | C |
| ATOM | 5901 | O | ALA | M | 112 | 58.390 | −1.328 | −11.668 | 1.00 | 18.35 | O |
| ATOM | 5902 | N | PRO | M | 113 | 58.775 | −1.938 | −13.817 | 1.00 | 18.19 | N |
| ATOM | 5903 | CA | PRO | M | 113 | 59.621 | −3.063 | −13.422 | 1.00 | 18.16 | C |
| ATOM | 5904 | CB | PRO | M | 113 | 59.712 | −3.892 | −14.705 | 1.00 | 18.47 | C |
| ATOM | 5905 | CG | PRO | M | 113 | 59.471 | −2.944 | −15.796 | 1.00 | 19.50 | C |
| ATOM | 5906 | CD | PRO | M | 113 | 58.521 | −1.917 | −15.270 | 1.00 | 17.68 | C |
| ATOM | 5907 | C | PRO | M | 113 | 61.018 | −2.605 | −13.007 | 1.00 | 18.38 | C |
| ATOM | 5908 | O | PRO | M | 113 | 61.535 | −1.638 | −13.576 | 1.00 | 17.31 | O |
| ATOM | 5909 | N | SER | M | 114 | 61.600 | −3.269 | −12.008 | 1.00 | 18.43 | N |
| ATOM | 5910 | CA | SER | M | 114 | 63.032 | −3.153 | −11.741 | 1.00 | 18.87 | C |
| ATOM | 5911 | CB | SER | M | 114 | 63.345 | −3.409 | −10.260 | 1.00 | 19.38 | C |
| ATOM | 5912 | OG | SER | M | 114 | 62.836 | −2.365 | −9.434 | 1.00 | 20.08 | O |
| ATOM | 5913 | C | SER | M | 114 | 63.711 | −4.207 | −12.603 | 1.00 | 18.89 | C |
| ATOM | 5914 | O | SER | M | 114 | 63.305 | −5.380 | −12.579 | 1.00 | 19.02 | O |
| ATOM | 5915 | N | VAL | M | 115 | 64.733 | −3.799 | −13.356 | 1.00 | 18.25 | N |
| ATOM | 5916 | CA | VAL | M | 115 | 65.328 | −4.666 | −14.382 | 1.00 | 17.85 | C |
| ATOM | 5917 | CB | VAL | M | 115 | 65.311 | −3.994 | −15.779 | 1.00 | 17.50 | C |
| ATOM | 5918 | CG1 | VAL | M | 115 | 65.795 | −4.962 | −16.840 | 1.00 | 17.96 | C |
| ATOM | 5919 | CG2 | VAL | M | 115 | 63.893 | −3.565 | −16.145 | 1.00 | 17.94 | C |
| ATOM | 5920 | C | VAL | M | 115 | 66.746 | −5.123 | −14.028 | 1.00 | 17.67 | C |
| ATOM | 5921 | O | VAL | M | 115 | 67.564 | −4.317 | −13.583 | 1.00 | 17.15 | O |
| ATOM | 5922 | N | PHE | M | 116 | 67.024 | −6.411 | −14.231 | 1.00 | 17.33 | N |
| ATOM | 5923 | CA | PHE | M | 116 | 68.354 | −6.979 | −13.958 | 1.00 | 18.06 | C |
| ATOM | 5924 | CB | PHE | M | 116 | 68.341 | −7.783 | −12.648 | 1.00 | 17.80 | C |
| ATOM | 5925 | CG | PHE | M | 116 | 67.850 | −7.005 | −11.463 | 1.00 | 18.51 | C |
| ATOM | 5926 | CD1 | PHE | M | 116 | 68.754 | −6.360 | −10.619 | 1.00 | 19.72 | C |
| ATOM | 5927 | CE1 | PHE | M | 116 | 68.298 | −5.637 | −9.513 | 1.00 | 19.59 | C |
| ATOM | 5928 | CZ | PHE | M | 116 | 66.935 | −5.547 | −9.252 | 1.00 | 20.33 | C |
| ATOM | 5929 | CE2 | PHE | M | 116 | 66.021 | −6.201 | −10.072 | 1.00 | 20.52 | C |
| ATOM | 5930 | CD2 | PHE | M | 116 | 66.479 | −6.922 | −11.175 | 1.00 | 19.67 | C |
| ATOM | 5931 | C | PHE | M | 116 | 68.779 | −7.910 | −15.077 | 1.00 | 18.12 | C |
| ATOM | 5932 | O | PHE | M | 116 | 67.970 | −8.711 | −15.554 | 1.00 | 17.70 | O |
| ATOM | 5933 | N | ILE | M | 117 | 70.051 | −7.836 | −15.468 | 1.00 | 17.49 | N |
| ATOM | 5934 | CA | ILE | M | 117 | 70.592 | −8.779 | −16.457 | 1.00 | 18.03 | C |
| ATOM | 5935 | CB | ILE | M | 117 | 71.167 | −8.047 | −17.703 | 1.00 | 18.21 | C |
| ATOM | 5936 | CG1 | ILE | M | 117 | 71.412 | −9.031 | −18.861 | 1.00 | 19.12 | C |
| ATOM | 5937 | CD1 | ILE | M | 117 | 71.565 | −8.367 | −20.219 | 1.00 | 18.78 | C |
| ATOM | 5938 | CG2 | ILE | M | 117 | 72.431 | −7.217 | −17.344 | 1.00 | 18.51 | C |
| ATOM | 5939 | C | ILE | M | 117 | 71.641 | −9.689 | −15.797 | 1.00 | 17.91 | C |
| ATOM | 5940 | O | ILE | M | 117 | 72.412 | −9.232 | −14.943 | 1.00 | 17.59 | O |
| ATOM | 5941 | N | PHE | M | 118 | 71.649 | −10.960 | −16.191 | 1.00 | 17.60 | N |
| ATOM | 5942 | CA | PHE | M | 118 | 72.615 | −11.937 | −15.697 | 1.00 | 18.32 | C |
| ATOM | 5943 | CB | PHE | M | 118 | 71.905 | −12.999 | −14.853 | 1.00 | 18.05 | C |
| ATOM | 5944 | CG | PHE | M | 118 | 71.155 | −12.451 | −13.657 | 1.00 | 17.97 | C |
| ATOM | 5945 | CD1 | PHE | M | 118 | 71.804 | −12.264 | −12.431 | 1.00 | 18.47 | C |
| ATOM | 5946 | CE1 | PHE | M | 118 | 71.107 | −11.765 | −11.305 | 1.00 | 18.74 | C |
| ATOM | 5947 | CZ | PHE | M | 118 | 69.744 | −11.466 | −11.408 | 1.00 | 19.20 | C |
| ATOM | 5948 | CE2 | PHE | M | 118 | 69.083 | −11.651 | −12.635 | 1.00 | 19.96 | C |
| ATOM | 5949 | CD2 | PHE | M | 118 | 69.797 | −12.153 | −13.749 | 1.00 | 18.37 | C |
| ATOM | 5950 | C | PHE | M | 118 | 73.368 | −12.633 | −16.850 | 1.00 | 18.93 | C |
| ATOM | 5951 | O | PHE | M | 118 | 72.742 | −13.211 | −17.753 | 1.00 | 18.90 | O |
| ATOM | 5952 | N | PRO | M | 119 | 74.716 | −12.599 | −16.816 | 1.00 | 19.55 | N |
| ATOM | 5953 | CA | PRO | M | 119 | 75.498 | −13.342 | −17.801 | 1.00 | 19.29 | C |
| ATOM | 5954 | CB | PRO | M | 119 | 76.936 | −12.847 | −17.552 | 1.00 | 19.92 | C |
| ATOM | 5955 | CG | PRO | M | 119 | 76.949 | −12.447 | −16.099 | 1.00 | 20.43 | C |
| ATOM | 5956 | CD | PRO | M | 119 | 75.581 | −11.826 | −15.899 | 1.00 | 19.96 | C |
| ATOM | 5957 | C | PRO | M | 119 | 75.412 | −14.848 | −17.550 | 1.00 | 19.09 | C |
| ATOM | 5958 | O | PRO | M | 119 | 75.066 | −15.262 | −16.430 | 1.00 | 18.68 | O |
| ATOM | 5959 | N | PRO | M | 120 | 75.722 | −15.674 | −18.575 | 1.00 | 18.64 | N |
| ATOM | 5960 | CA | PRO | M | 120 | 75.811 | −17.099 | −18.287 | 1.00 | 18.81 | C |
| ATOM | 5961 | CB | PRO | M | 120 | 76.125 | −17.724 | −19.654 | 1.00 | 18.87 | C |
| ATOM | 5962 | CG | PRO | M | 120 | 76.620 | −16.608 | −20.498 | 1.00 | 18.39 | C |
| ATOM | 5963 | CD | PRO | M | 120 | 75.976 | −15.374 | −19.998 | 1.00 | 18.88 | C |
| ATOM | 5964 | C | PRO | M | 120 | 76.948 | −17.350 | −17.301 | 1.00 | 19.14 | C |
| ATOM | 5965 | O | PRO | M | 120 | 77.961 | −16.647 | −17.328 | 1.00 | 18.66 | O |
| ATOM | 5966 | N | SER | M | 121 | 76.762 | −18.315 | −16.412 | 1.00 | 19.68 | N |
| ATOM | 5967 | CA | SER | M | 121 | 77.824 | −18.686 | −15.487 | 1.00 | 20.30 | C |
| ATOM | 5968 | CB | SER | M | 121 | 77.291 | −19.600 | −14.385 | 1.00 | 20.05 | C |
| ATOM | 5969 | OG | SER | M | 121 | 76.785 | −20.811 | −14.914 | 1.00 | 19.57 | O |
| ATOM | 5970 | C | SER | M | 121 | 78.971 | −19.358 | −16.238 | 1.00 | 21.00 | C |
| ATOM | 5971 | O | SER | M | 121 | 78.778 | −19.906 | −17.333 | 1.00 | 20.67 | O |
| ATOM | 5972 | N | ASP | M | 122 | 80.164 | −19.307 | −15.649 | 1.00 | 21.76 | N |
| ATOM | 5973 | CA | ASP | M | 122 | 81.315 | −20.008 | −16.208 | 1.00 | 22.62 | C |
| ATOM | 5974 | CB | ASP | M | 122 | 82.598 | −19.638 | −15.448 | 1.00 | 23.15 | C |
| ATOM | 5975 | CG | ASP | M | 122 | 83.058 | −18.196 | −15.710 | 1.00 | 25.04 | C |
| ATOM | 5976 | OD1 | ASP | M | 122 | 82.351 | −17.410 | −16.394 | 1.00 | 27.18 | O |
| ATOM | 5977 | OD2 | ASP | M | 122 | 84.150 | −17.850 | −15.223 | 1.00 | 26.57 | O |
| ATOM | 5978 | C | ASP | M | 122 | 81.094 | −21.528 | −16.200 | 1.00 | 22.64 | C |
| ATOM | 5979 | O | ASP | M | 122 | 81.640 | −22.244 | −17.041 | 1.00 | 22.29 | O |
| ATOM | 5980 | N | GLU | M | 123 | 80.283 | −22.005 | −15.254 | 1.00 | 23.01 | N |
| ATOM | 5981 | CA | GLU | M | 123 | 79.951 | −23.424 | −15.165 | 1.00 | 23.54 | C |
| ATOM | 5982 | CB | GLU | M | 123 | 79.176 | −23.750 | −13.880 | 1.00 | 23.84 | C |
| ATOM | 5983 | CG | GLU | M | 123 | 79.322 | −25.216 | −13.472 | 1.00 | 26.39 | C |
| ATOM | 5984 | CD | GLU | M | 123 | 78.217 | −25.709 | −12.555 | 1.00 | 29.83 | C |
| ATOM | 5985 | OE1 | GLU | M | 123 | 77.658 | −24.894 | −11.778 | 1.00 | 32.64 | O |
| ATOM | 5986 | OE2 | GLU | M | 123 | 77.900 | −26.920 | −12.619 | 1.00 | 30.31 | O |
| ATOM | 5987 | C | GLU | M | 123 | 79.149 | −23.886 | −16.382 | 1.00 | 23.08 | C |
| ATOM | 5988 | O | GLU | M | 123 | 79.385 | −24.979 | −16.903 | 1.00 | 22.66 | O |
| ATOM | 5989 | N | GLN | M | 124 | 78.200 | −23.058 | −16.821 | 1.00 | 22.74 | N |
| ATOM | 5990 | CA | GLN | M | 124 | 77.386 | −23.404 | −17.986 | 1.00 | 22.50 | C |
| ATOM | 5991 | CB | GLN | M | 124 | 76.170 | −22.478 | −18.158 | 1.00 | 21.69 | C |
| ATOM | 5992 | CG | GLN | M | 124 | 75.218 | −22.972 | −19.261 | 1.00 | 19.92 | C |
| ATOM | 5993 | CD | GLN | M | 124 | 74.014 | −22.076 | −19.518 | 1.00 | 18.87 | C |
| ATOM | 5994 | OE1 | GLN | M | 124 | 73.991 | −20.904 | −19.141 | 1.00 | 17.80 | O |
| ATOM | 5995 | NE2 | GLN | M | 124 | 73.001 | −22.636 | −20.171 | 1.00 | 16.89 | N |
| ATOM | 5996 | C | GLN | M | 124 | 78.217 | −23.405 | −19.262 | 1.00 | 22.91 | C |
| ATOM | 5997 | O | GLN | M | 124 | 78.077 | −24.301 | −20.087 | 1.00 | 22.54 | O |
| ATOM | 5998 | N | LEU | M | 125 | 79.063 | −22.390 | −19.420 | 1.00 | 23.91 | N |
| ATOM | 5999 | CA | LEU | M | 125 | 79.877 | −22.252 | −20.627 | 1.00 | 25.24 | C |
| ATOM | 6000 | CB | LEU | M | 125 | 80.801 | −21.044 | −20.526 | 1.00 | 25.44 | C |
| ATOM | 6001 | CG | LEU | M | 125 | 80.128 | −19.676 | −20.636 | 1.00 | 25.92 | C |
| ATOM | 6002 | CD1 | LEU | M | 125 | 79.165 | −19.632 | −21.813 | 1.00 | 26.75 | C |
| ATOM | 6003 | CD2 | LEU | M | 125 | 81.190 | −18.596 | −20.779 | 1.00 | 27.85 | C |
| ATOM | 6004 | C | LEU | M | 125 | 80.664 | −23.512 | −20.958 | 1.00 | 25.94 | C |
| ATOM | 6005 | O | LEU | M | 125 | 80.700 | −23.922 | −22.118 | 1.00 | 26.38 | O |
| ATOM | 6006 | N | LYS | M | 126 | 81.258 | −24.134 | −19.935 | 1.00 | 26.97 | N |
| ATOM | 6007 | CA | LYS | M | 126 | 81.985 | −25.410 | −20.077 | 1.00 | 27.82 | C |
| ATOM | 6008 | CB | LYS | M | 126 | 82.372 | −25.994 | −18.711 | 1.00 | 28.21 | C |
| ATOM | 6009 | CG | LYS | M | 126 | 83.236 | −25.138 | −17.786 | 1.00 | 29.68 | C |
| ATOM | 6010 | CD | LYS | M | 126 | 83.565 | −25.943 | −16.502 | 1.00 | 31.65 | C |
| ATOM | 6011 | CE | LYS | M | 126 | 82.318 | −26.155 | −15.629 | 1.00 | 31.93 | C |
| ATOM | 6012 | NZ | LYS | M | 126 | 82.439 | −27.276 | −14.652 | 1.00 | 32.13 | N |
| ATOM | 6013 | C | LYS | M | 126 | 81.176 | −26.489 | −20.804 | 1.00 | 27.83 | C |
| ATOM | 6014 | O | LYS | M | 126 | 81.753 | −27.377 | −21.431 | 1.00 | 27.91 | O |
| ATOM | 6015 | N | SER | M | 127 | 79.849 | −26.427 | −20.696 | 1.00 | 27.68 | N |
| ATOM | 6016 | CA | SER | M | 127 | 78.987 | −27.450 | −21.293 | 1.00 | 27.69 | C |
| ATOM | 6017 | CB | SER | M | 127 | 77.788 | −27.751 | −20.389 | 1.00 | 27.75 | C |
| ATOM | 6018 | OG | SER | M | 127 | 76.963 | −26.610 | −20.226 | 1.00 | 29.35 | O |
| ATOM | 6019 | C | SER | M | 127 | 78.540 | −27.133 | −22.732 | 1.00 | 27.22 | C |
| ATOM | 6020 | O | SER | M | 127 | 77.810 | −27.914 | −23.353 | 1.00 | 27.29 | O |
| ATOM | 6021 | N | GLY | M | 128 | 78.987 | −25.996 | −23.260 | 1.00 | 26.52 | N |
| ATOM | 6022 | CA | GLY | M | 128 | 78.750 | −25.656 | −24.666 | 1.00 | 25.53 | C |
| ATOM | 6023 | C | GLY | M | 128 | 77.467 | −24.892 | −24.952 | 1.00 | 24.63 | C |
| ATOM | 6024 | O | GLY | M | 128 | 77.000 | −24.862 | −26.096 | 1.00 | 24.38 | O |
| ATOM | 6025 | N | THR | M | 129 | 76.908 | −24.264 | −23.918 | 1.00 | 23.83 | N |
| ATOM | 6026 | CA | THR | M | 129 | 75.690 | −23.447 | −24.047 | 1.00 | 22.94 | C |
| ATOM | 6027 | CB | THR | M | 129 | 74.429 | −24.266 | −23.686 | 1.00 | 23.17 | C |
| ATOM | 6028 | OG1 | THR | M | 129 | 74.371 | −25.427 | −24.516 | 1.00 | 23.09 | O |
| ATOM | 6029 | CG2 | THR | M | 129 | 73.140 | −23.462 | −23.893 | 1.00 | 22.66 | C |
| ATOM | 6030 | C | THR | M | 129 | 75.772 | −22.207 | −23.157 | 1.00 | 22.49 | C |
| ATOM | 6031 | O | THR | M | 129 | 76.409 | −22.235 | −22.093 | 1.00 | 22.54 | O |
| ATOM | 6032 | N | ALA | M | 130 | 75.137 | −21.124 | −23.604 | 1.00 | 21.16 | N |
| ATOM | 6033 | CA | ALA | M | 130 | 75.059 | −19.889 | −22.839 | 1.00 | 20.13 | C |
| ATOM | 6034 | CB | ALA | M | 130 | 75.900 | −18.818 | −23.494 | 1.00 | 19.93 | C |
| ATOM | 6035 | C | ALA | M | 130 | 73.609 | −19.417 | −22.716 | 1.00 | 19.70 | C |
| ATOM | 6036 | O | ALA | M | 130 | 72.942 | −19.168 | −23.730 | 1.00 | 19.53 | O |
| ATOM | 6037 | N | SER | M | 131 | 73.122 | −19.318 | −21.480 | 1.00 | 18.76 | N |
| ATOM | 6038 | CA | SER | M | 131 | 71.826 | −18.683 | −21.212 | 1.00 | 18.54 | C |
| ATOM | 6039 | CB | SER | M | 131 | 70.942 | −19.542 | −20.296 | 1.00 | 18.16 | C |
| ATOM | 6040 | OG | SER | M | 131 | 70.857 | −20.875 | −20.759 | 1.00 | 17.58 | O |
| ATOM | 6041 | C | SER | M | 131 | 72.053 | −17.323 | −20.576 | 1.00 | 18.45 | C |
| ATOM | 6042 | O | SER | M | 131 | 72.778 | −17.200 | −19.576 | 1.00 | 18.48 | O |
| ATOM | 6043 | N | VAL | M | 132 | 71.452 | −16.304 | −21.177 | 1.00 | 17.86 | N |
| ATOM | 6044 | CA | VAL | M | 132 | 71.487 | −14.945 | −20.643 | 1.00 | 17.33 | C |
| ATOM | 6045 | CB | VAL | M | 132 | 71.883 | −13.915 | −21.730 | 1.00 | 17.26 | C |
| ATOM | 6046 | CG1 | VAL | M | 132 | 72.250 | −12.566 | −21.104 | 1.00 | 17.24 | C |
| ATOM | 6047 | CG2 | VAL | M | 132 | 73.058 | −14.429 | −22.560 | 1.00 | 17.25 | C |
| ATOM | 6048 | C | VAL | M | 132 | 70.080 | −14.649 | −20.131 | 1.00 | 17.40 | C |
| ATOM | 6049 | O | VAL | M | 132 | 69.101 | −14.831 | −20.859 | 1.00 | 16.96 | O |
| ATOM | 6050 | N | VAL | M | 133 | 69.974 | −14.223 | −18.873 | 1.00 | 17.24 | N |
| ATOM | 6051 | CA | VAL | M | 133 | 68.667 | −14.027 | −18.253 | 1.00 | 17.49 | C |
| ATOM | 6052 | CB | VAL | M | 133 | 68.502 | −14.878 | −16.962 | 1.00 | 17.35 | C |
| ATOM | 6053 | CG1 | VAL | M | 133 | 67.143 | −14.605 | −16.280 | 1.00 | 17.76 | C |
| ATOM | 6054 | CG2 | VAL | M | 133 | 68.651 | −16.381 | −17.283 | 1.00 | 17.25 | C |
| ATOM | 6055 | C | VAL | M | 133 | 68.417 | −12.557 | −17.968 | 1.00 | 17.82 | C |
| ATOM | 6056 | O | VAL | M | 133 | 69.304 | −11.839 | −17.493 | 1.00 | 17.59 | O |
| ATOM | 6057 | N | CYS | M | 134 | 67.202 | −12.126 | −18.274 | 1.00 | 18.20 | N |
| ATOM | 6058 | CA | CYS | M | 134 | 66.733 | −10.789 | −17.954 | 1.00 | 18.93 | C |
| ATOM | 6059 | CB | CYS | M | 134 | 66.350 | −10.034 | −19.221 | 1.00 | 19.01 | C |
| ATOM | 6060 | SG | CYS | M | 134 | 65.881 | −8.331 | −18.907 | 1.00 | 22.07 | S |
| ATOM | 6061 | C | CYS | M | 134 | 65.520 | −10.874 | −17.024 | 1.00 | 18.66 | C |
| ATOM | 6062 | O | CYS | M | 134 | 64.509 | −11.512 | −17.354 | 1.00 | 19.02 | O |
| ATOM | 6063 | N | LEU | M | 135 | 65.615 | −10.217 | −15.874 | 1.00 | 17.99 | N |
| ATOM | 6064 | CA | LEU | M | 135 | 64.532 | −10.255 | −14.894 | 1.00 | 17.28 | C |
| ATOM | 6065 | CB | LEU | M | 135 | 65.098 | −10.590 | −13.502 | 1.00 | 17.42 | C |
| ATOM | 6066 | CG | LEU | M | 135 | 64.211 | −10.280 | −12.275 | 1.00 | 17.42 | C |
| ATOM | 6067 | CD1 | LEU | M | 135 | 62.994 | −11.185 | −12.227 | 1.00 | 18.32 | C |
| ATOM | 6068 | CD2 | LEU | M | 135 | 64.991 | −10.413 | −10.983 | 1.00 | 18.48 | C |
| ATOM | 6069 | C | LEU | M | 135 | 63.823 | −8.912 | −14.864 | 1.00 | 17.07 | C |
| ATOM | 6070 | O | LEU | M | 135 | 64.491 | −7.870 | −14.815 | 1.00 | 16.18 | O |
| ATOM | 6071 | N | LEU | M | 136 | 62.489 | −8.939 | −14.904 | 1.00 | 17.10 | N |
| ATOM | 6072 | CA | LEU | M | 136 | 61.643 | −7.744 | −14.711 | 1.00 | 17.45 | C |
| ATOM | 6073 | CB | LEU | M | 136 | 60.651 | −7.558 | −15.865 | 1.00 | 17.56 | C |
| ATOM | 6074 | CG | LEU | M | 136 | 61.137 | −7.008 | −17.213 | 1.00 | 17.83 | C |
| ATOM | 6075 | CD1 | LEU | M | 136 | 62.228 | −7.894 | −17.848 | 1.00 | 18.65 | C |
| ATOM | 6076 | CD2 | LEU | M | 136 | 59.950 | −6.878 | −18.166 | 1.00 | 17.35 | C |
| ATOM | 6077 | C | LEU | M | 136 | 60.860 | −7.957 | −13.442 | 1.00 | 18.11 | C |
| ATOM | 6078 | O | LEU | M | 136 | 59.989 | −8.823 | −13.387 | 1.00 | 18.45 | O |
| ATOM | 6079 | N | ASN | M | 137 | 61.173 | −7.187 | −12.414 | 1.00 | 17.88 | N |
| ATOM | 6080 | CA | ASN | M | 137 | 60.651 | −7.492 | −11.097 | 1.00 | 18.76 | C |
| ATOM | 6081 | CB | ASN | M | 137 | 61.787 | −7.526 | −10.075 | 1.00 | 18.45 | C |
| ATOM | 6082 | CG | ASN | M | 137 | 61.508 | −8.492 | −8.964 | 1.00 | 21.59 | C |
| ATOM | 6083 | OD1 | ASN | M | 137 | 61.104 | −9.629 | −9.212 | 1.00 | 25.07 | O |
| ATOM | 6084 | ND2 | ASN | M | 137 | 61.702 | −8.056 | −7.731 | 1.00 | 20.71 | N |
| ATOM | 6085 | C | ASN | M | 137 | 59.577 | −6.533 | −10.637 | 1.00 | 18.16 | C |
| ATOM | 6086 | O | ASN | M | 137 | 59.784 | −5.322 | −10.677 | 1.00 | 17.58 | O |
| ATOM | 6087 | N | ASN | M | 138 | 58.443 | −7.090 | −10.211 | 1.00 | 18.19 | N |
| ATOM | 6088 | CA | ASN | M | 138 | 57.401 | −6.366 | −9.463 | 1.00 | 18.19 | C |
| ATOM | 6089 | CB | ASN | M | 138 | 57.967 | −5.885 | −8.114 | 1.00 | 18.68 | C |
| ATOM | 6090 | CG | ASN | M | 138 | 58.200 | −7.025 | −7.146 | 1.00 | 20.11 | C |
| ATOM | 6091 | OD1 | ASN | M | 138 | 57.932 | −8.197 | −7.446 | 1.00 | 22.50 | O |
| ATOM | 6092 | ND2 | ASN | M | 138 | 58.699 | −6.691 | −5.973 | 1.00 | 23.52 | N |
| ATOM | 6093 | C | ASN | M | 138 | 56.718 | −5.213 | −10.200 | 1.00 | 17.65 | C |
| ATOM | 6094 | O | ASN | M | 138 | 56.789 | −4.061 | −9.763 | 1.00 | 17.57 | O |
| ATOM | 6095 | N | PHE | M | 139 | 56.053 | −5.519 | −11.307 | 1.00 | 17.43 | N |
| ATOM | 6096 | CA | PHE | M | 139 | 55.461 | −4.481 | −12.157 | 1.00 | 17.22 | C |
| ATOM | 6097 | CB | PHE | M | 139 | 56.192 | −4.406 | −13.517 | 1.00 | 17.42 | C |
| ATOM | 6098 | CG | PHE | M | 139 | 56.152 | −5.687 | −14.301 | 1.00 | 17.84 | C |
| ATOM | 6099 | CD1 | PHE | M | 139 | 57.166 | −6.638 | −14.158 | 1.00 | 17.73 | C |
| ATOM | 6100 | CE1 | PHE | M | 139 | 57.132 | −7.845 | −14.884 | 1.00 | 19.40 | C |
| ATOM | 6101 | CZ | PHE | M | 139 | 56.069 | −8.091 | −15.773 | 1.00 | 18.43 | C |
| ATOM | 6102 | CE2 | PHE | M | 139 | 55.052 | −7.128 | −15.925 | 1.00 | 19.57 | C |
| ATOM | 6103 | CD2 | PHE | M | 139 | 55.102 | −5.936 | −15.194 | 1.00 | 17.50 | C |
| ATOM | 6104 | C | PHE | M | 139 | 53.956 | −4.652 | −12.357 | 1.00 | 17.44 | C |
| ATOM | 6105 | O | PHE | M | 139 | 53.416 | −5.713 | −12.076 | 1.00 | 16.84 | O |
| ATOM | 6106 | N | TYR | M | 140 | 53.295 | −3.596 | −12.845 | 1.00 | 17.59 | N |
| ATOM | 6107 | CA | TYR | M | 140 | 51.861 | −3.612 | −13.136 | 1.00 | 17.75 | C |
| ATOM | 6108 | CB | TYR | M | 140 | 50.996 | −3.395 | −11.865 | 1.00 | 17.89 | C |
| ATOM | 6109 | CG | TYR | M | 140 | 49.523 | −3.615 | −12.146 | 1.00 | 17.92 | C |
| ATOM | 6110 | CD1 | TYR | M | 140 | 48.742 | −2.604 | −12.718 | 1.00 | 18.29 | C |
| ATOM | 6111 | CE1 | TYR | M | 140 | 47.406 | −2.811 | −13.022 | 1.00 | 16.74 | C |
| ATOM | 6112 | CZ | TYR | M | 140 | 46.823 | −4.050 | −12.737 | 1.00 | 18.06 | C |
| ATOM | 6113 | OH | TYR | M | 140 | 45.497 | −4.269 | −13.053 | 1.00 | 14.46 | O |
| ATOM | 6114 | CE2 | TYR | M | 140 | 47.580 | −5.075 | −12.199 | 1.00 | 17.35 | C |
| ATOM | 6115 | CD2 | TYR | M | 140 | 48.928 | −4.851 | −11.907 | 1.00 | 17.68 | C |
| ATOM | 6116 | C | TYR | M | 140 | 51.550 | −2.538 | −14.181 | 1.00 | 18.14 | C |
| ATOM | 6117 | O | TYR | M | 140 | 52.045 | −1.414 | −14.068 | 1.00 | 17.64 | O |
| ATOM | 6118 | N | PRO | M | 141 | 50.710 | −2.861 | −15.190 | 1.00 | 18.32 | N |
| ATOM | 6119 | CA | PRO | M | 141 | 50.039 | −4.137 | −15.423 | 1.00 | 18.57 | C |
| ATOM | 6120 | CB | PRO | M | 141 | 48.876 | −3.748 | −16.360 | 1.00 | 18.61 | C |
| ATOM | 6121 | CG | PRO | M | 141 | 49.417 | −2.631 | −17.140 | 1.00 | 18.43 | C |
| ATOM | 6122 | CD | PRO | M | 141 | 50.324 | −1.863 | −16.203 | 1.00 | 17.66 | C |
| ATOM | 6123 | C | PRO | M | 141 | 50.956 | −5.190 | −16.061 | 1.00 | 19.10 | C |
| ATOM | 6124 | O | PRO | M | 141 | 52.161 | −4.945 | −16.240 | 1.00 | 18.05 | O |
| ATOM | 6125 | N | ARG | M | 142 | 50.376 | −6.355 | −16.378 | 1.00 | 19.73 | N |
| ATOM | 6126 | CA | ARG | M | 142 | 51.120 | −7.531 | −16.852 | 1.00 | 20.67 | C |
| ATOM | 6127 | CB | ARG | M | 142 | 50.156 | −8.727 | −16.993 | 1.00 | 20.88 | C |
| ATOM | 6128 | CG | ARG | M | 142 | 50.805 | −10.082 | −17.274 | 1.00 | 24.27 | C |
| ATOM | 6129 | CD | ARG | M | 142 | 49.742 | −11.210 | −17.372 | 1.00 | 28.79 | C |
| ATOM | 6130 | NE | ARG | M | 142 | 50.209 | −12.368 | −16.618 | 1.00 | 34.80 | N |
| ATOM | 6131 | CZ | ARG | M | 142 | 49.558 | −12.983 | −15.622 | 1.00 | 34.86 | C |
| ATOM | 6132 | NH1 | ARG | M | 142 | 48.328 | −12.601 | −15.258 | 1.00 | 34.15 | N |
| ATOM | 6133 | NH2 | ARG | M | 142 | 50.151 | −14.002 | −14.997 | 1.00 | 32.02 | N |
| ATOM | 6134 | C | ARG | M | 142 | 51.870 | −7.299 | −18.165 | 1.00 | 20.89 | C |
| ATOM | 6135 | O | ARG | M | 142 | 52.978 | −7.807 | −18.346 | 1.00 | 20.71 | O |
| ATOM | 6136 | N | GLU | M | 143 | 51.250 | −6.566 | −19.080 | 1.00 | 21.19 | N |
| ATOM | 6137 | CA | GLU | M | 143 | 51.815 | −6.285 | −20.395 | 1.00 | 22.28 | C |
| ATOM | 6138 | CB | GLU | M | 143 | 50.850 | −5.391 | −21.183 | 1.00 | 22.60 | C |
| ATOM | 6139 | CG | GLU | M | 143 | 49.506 | −6.064 | −21.551 | 1.00 | 24.28 | C |
| ATOM | 6140 | CD | GLU | M | 143 | 48.561 | −6.357 | −20.371 | 1.00 | 26.31 | C |
| ATOM | 6141 | OE1 | GLU | M | 143 | 48.515 | −5.583 | −19.384 | 1.00 | 23.70 | O |
| ATOM | 6142 | OE2 | GLU | M | 143 | 47.835 | −7.383 | −20.446 | 1.00 | 25.39 | O |
| ATOM | 6143 | C | GLU | M | 143 | 53.197 | −5.618 | −20.285 | 1.00 | 22.57 | C |
| ATOM | 6144 | O | GLU | M | 143 | 53.346 | −4.628 | −19.592 | 1.00 | 22.53 | O |
| ATOM | 6145 | N | ALA | M | 144 | 54.196 | −6.203 | −20.942 | 1.00 | 22.93 | N |
| ATOM | 6146 | CA | ALA | M | 144 | 55.581 | −5.708 | −20.951 | 1.00 | 23.22 | C |
| ATOM | 6147 | CB | ALA | M | 144 | 56.313 | −6.124 | −19.685 | 1.00 | 23.44 | C |
| ATOM | 6148 | C | ALA | M | 144 | 56.279 | −6.305 | −22.169 | 1.00 | 24.00 | C |
| ATOM | 6149 | O | ALA | M | 144 | 55.938 | −7.419 | −22.594 | 1.00 | 24.76 | O |
| ATOM | 6150 | N | LYS | M | 145 | 57.254 | −5.590 | −22.724 | 1.00 | 23.51 | N |
| ATOM | 6151 | CA | LYS | M | 145 | 57.940 | −6.059 | −23.915 | 1.00 | 23.90 | C |
| ATOM | 6152 | CB | LYS | M | 145 | 57.572 | −5.169 | −25.104 | 1.00 | 23.78 | C |
| ATOM | 6153 | CG | LYS | M | 145 | 58.553 | −5.150 | −26.265 | 1.00 | 25.63 | C |
| ATOM | 6154 | CD | LYS | M | 145 | 58.043 | −4.211 | −27.355 | 1.00 | 28.45 | C |
| ATOM | 6155 | CE | LYS | M | 145 | 59.064 | −3.991 | −28.454 | 1.00 | 28.60 | C |
| ATOM | 6156 | NZ | LYS | M | 145 | 58.648 | −2.897 | −29.393 | 1.00 | 30.84 | N |
| ATOM | 6157 | C | LYS | M | 145 | 59.447 | −6.113 | −23.686 | 1.00 | 23.65 | C |
| ATOM | 6158 | O | LYS | M | 145 | 60.078 | −5.094 | −23.364 | 1.00 | 23.05 | O |
| ATOM | 6159 | N | VAL | M | 146 | 60.007 | −7.311 | −23.837 | 1.00 | 23.16 | N |
| ATOM | 6160 | CA | VAL | M | 146 | 61.452 | −7.513 | −23.732 | 1.00 | 23.04 | C |
| ATOM | 6161 | CB | VAL | M | 146 | 61.799 | −8.749 | −22.866 | 1.00 | 23.60 | C |
| ATOM | 6162 | CG1 | VAL | M | 146 | 63.312 | −8.914 | −22.757 | 1.00 | 23.21 | C |
| ATOM | 6163 | CG2 | VAL | M | 146 | 61.159 | −8.649 | −21.479 | 1.00 | 23.15 | C |
| ATOM | 6164 | C | VAL | M | 146 | 62.059 | −7.708 | −25.121 | 1.00 | 23.17 | C |
| ATOM | 6165 | O | VAL | M | 146 | 61.611 | −8.574 | −25.871 | 1.00 | 22.79 | O |
| ATOM | 6166 | N | GLN | M | 147 | 63.067 | −6.900 | −25.459 | 1.00 | 22.87 | N |
| ATOM | 6167 | CA | GLN | M | 147 | 63.837 | −7.091 | −26.695 | 1.00 | 23.34 | C |
| ATOM | 6168 | CB | GLN | M | 147 | 63.758 | −5.866 | −27.616 | 1.00 | 23.80 | C |
| ATOM | 6169 | CG | GLN | M | 147 | 62.364 | −5.617 | −28.195 | 1.00 | 27.03 | C |
| ATOM | 6170 | CD | GLN | M | 147 | 62.370 | −4.706 | −29.425 | 1.00 | 31.15 | C |
| ATOM | 6171 | OE1 | GLN | M | 147 | 61.667 | −4.971 | −30.399 | 1.00 | 33.32 | O |
| ATOM | 6172 | NE2 | GLN | M | 147 | 63.160 | −3.630 | −29.382 | 1.00 | 32.86 | N |
| ATOM | 6173 | C | GLN | M | 147 | 65.289 | −7.369 | −26.350 | 1.00 | 22.70 | C |
| ATOM | 6174 | O | GLN | M | 147 | 65.914 | −6.622 | −25.577 | 1.00 | 22.39 | O |
| ATOM | 6175 | N | TRP | M | 148 | 65.818 | −8.451 | −26.906 | 1.00 | 22.20 | N |
| ATOM | 6176 | CA | TRP | M | 148 | 67.227 | −8.778 | −26.718 | 1.00 | 22.04 | C |
| ATOM | 6177 | CB | TRP | M | 148 | 67.443 | −10.285 | −26.670 | 1.00 | 21.75 | C |
| ATOM | 6178 | CG | TRP | M | 148 | 67.011 | −10.914 | −25.382 | 1.00 | 20.74 | C |
| ATOM | 6179 | CD1 | TRP | M | 148 | 65.828 | −11.536 | −25.135 | 1.00 | 19.32 | C |
| ATOM | 6180 | NE1 | TRP | M | 148 | 65.796 | −12.000 | −23.840 | 1.00 | 19.67 | N |
| ATOM | 6181 | CE2 | TRP | M | 148 | 66.972 | −11.668 | −23.220 | 1.00 | 20.05 | C |
| ATOM | 6182 | CD2 | TRP | M | 148 | 67.764 | −10.982 | −24.162 | 1.00 | 20.62 | C |
| ATOM | 6183 | CE3 | TRP | M | 148 | 69.034 | −10.533 | −23.777 | 1.00 | 19.97 | C |
| ATOM | 6184 | CZ3 | TRP | M | 148 | 69.464 | −10.792 | −22.475 | 1.00 | 20.64 | C |
| ATOM | 6185 | CH2 | TRP | M | 148 | 68.655 | −11.472 | −21.566 | 1.00 | 19.56 | C |
| ATOM | 6186 | CZ2 | TRP | M | 148 | 67.407 | −11.920 | −21.914 | 1.00 | 20.47 | C |
| ATOM | 6187 | C | TRP | M | 148 | 68.048 | −8.171 | −27.832 | 1.00 | 22.23 | C |
| ATOM | 6188 | O | TRP | M | 148 | 67.636 | −8.163 | −28.992 | 1.00 | 22.10 | O |
| ATOM | 6189 | N | LYS | M | 149 | 69.211 | −7.645 | −27.464 | 1.00 | 22.58 | N |
| ATOM | 6190 | CA | LYS | M | 149 | 70.157 | −7.116 | −28.436 | 1.00 | 22.70 | C |
| ATOM | 6191 | CB | LYS | M | 149 | 70.115 | −5.591 | −28.456 | 1.00 | 23.35 | C |
| ATOM | 6192 | CG | LYS | M | 149 | 68.980 | −5.022 | −29.314 | 1.00 | 25.48 | C |
| ATOM | 6193 | CD | LYS | M | 149 | 68.933 | −3.493 | −29.265 | 1.00 | 28.83 | C |
| ATOM | 6194 | CE | LYS | M | 149 | 67.964 | −3.000 | −28.183 | 1.00 | 30.21 | C |
| ATOM | 6195 | NZ | LYS | M | 149 | 67.690 | −1.540 | −28.315 | 1.00 | 30.67 | N |
| ATOM | 6196 | C | LYS | M | 149 | 71.567 | −7.600 | −28.159 | 1.00 | 22.66 | C |
| ATOM | 6197 | O | LYS | M | 149 | 72.056 | −7.529 | −27.026 | 1.00 | 22.33 | O |
| ATOM | 6198 | N | VAL | M | 150 | 72.213 | −8.087 | −29.216 | 1.00 | 22.83 | N |
| ATOM | 6199 | CA | VAL | M | 150 | 73.572 | −8.596 | −29.163 | 1.00 | 22.75 | C |
| ATOM | 6200 | CB | VAL | M | 150 | 73.606 | −10.101 | −29.535 | 1.00 | 22.92 | C |
| ATOM | 6201 | CG1 | VAL | M | 150 | 75.031 | −10.606 | −29.610 | 1.00 | 22.13 | C |
| ATOM | 6202 | CG2 | VAL | M | 150 | 72.802 | −10.927 | −28.523 | 1.00 | 21.86 | C |
| ATOM | 6203 | C | VAL | M | 150 | 74.424 | −7.763 | −30.128 | 1.00 | 23.00 | C |
| ATOM | 6204 | O | VAL | M | 150 | 74.175 | −7.755 | −31.337 | 1.00 | 22.70 | O |
| ATOM | 6205 | N | ASP | M | 151 | 75.407 | −7.046 | −29.582 | 1.00 | 23.33 | N |
| ATOM | 6206 | CA | ASP | M | 151 | 76.163 | −6.039 | −30.337 | 1.00 | 23.94 | C |
| ATOM | 6207 | CB | ASP | M | 151 | 77.152 | −6.704 | −31.312 | 1.00 | 23.74 | C |
| ATOM | 6208 | CG | ASP | M | 151 | 78.392 | −7.263 | −30.608 | 1.00 | 24.74 | C |
| ATOM | 6209 | OD1 | ASP | M | 151 | 78.665 | −6.860 | −29.454 | 1.00 | 23.74 | O |
| ATOM | 6210 | OD2 | ASP | M | 151 | 79.106 | −8.094 | −31.220 | 1.00 | 26.04 | O |
| ATOM | 6211 | C | ASP | M | 151 | 75.225 | −5.057 | −31.062 | 1.00 | 24.34 | C |
| ATOM | 6212 | O | ASP | M | 151 | 75.473 | −4.668 | −32.204 | 1.00 | 24.14 | O |
| ATOM | 6213 | N | ASN | M | 152 | 74.144 | −4.671 | −30.377 | 1.00 | 25.03 | N |
| ATOM | 6214 | CA | ASN | M | 152 | 73.102 | −3.772 | −30.921 | 1.00 | 25.59 | C |
| ATOM | 6215 | CB | ASN | M | 152 | 73.670 | −2.367 | −31.236 | 1.00 | 25.94 | C |
| ATOM | 6216 | CG | ASN | M | 152 | 72.611 | −1.260 | −31.132 | 1.00 | 26.76 | C |
| ATOM | 6217 | OD1 | ASN | M | 152 | 71.731 | −1.298 | −30.269 | 1.00 | 28.27 | O |
| ATOM | 6218 | ND2 | ASN | M | 152 | 72.696 | −0.278 | −32.017 | 1.00 | 27.58 | N |
| ATOM | 6219 | C | ASN | M | 152 | 72.301 | −4.336 | −32.116 | 1.00 | 25.57 | C |
| ATOM | 6220 | O | ASN | M | 152 | 71.688 | −3.577 | −32.875 | 1.00 | 26.03 | O |
| ATOM | 6221 | N | ALA | M | 153 | 72.304 | −5.660 | −32.275 | 1.00 | 25.36 | N |
| ATOM | 6222 | CA | ALA | M | 153 | 71.456 | −6.323 | −33.261 | 1.00 | 25.05 | C |
| ATOM | 6223 | CB | ALA | M | 153 | 72.246 | −7.358 | −34.049 | 1.00 | 25.03 | C |
| ATOM | 6224 | C | ALA | M | 153 | 70.261 | −6.966 | −32.557 | 1.00 | 25.27 | C |
| ATOM | 6225 | O | ALA | M | 153 | 70.428 | −7.664 | −31.542 | 1.00 | 24.61 | O |
| ATOM | 6226 | N | LEU | M | 154 | 69.063 | −6.709 | −33.089 | 1.00 | 25.25 | N |
| ATOM | 6227 | CA | LEU | M | 154 | 67.825 | −7.237 | −32.522 | 1.00 | 25.78 | C |
| ATOM | 6228 | CB | LEU | M | 154 | 66.602 | −6.521 | −33.110 | 1.00 | 26.22 | C |
| ATOM | 6229 | CG | LEU | M | 154 | 65.932 | −5.453 | −32.229 | 1.00 | 27.60 | C |
| ATOM | 6230 | CD1 | LEU | M | 154 | 66.734 | −4.150 | −32.186 | 1.00 | 27.64 | C |
| ATOM | 6231 | CD2 | LEU | M | 154 | 64.494 | −5.180 | −32.708 | 1.00 | 29.20 | C |
| ATOM | 6232 | C | LEU | M | 154 | 67.715 | −8.743 | −32.716 | 1.00 | 25.63 | C |
| ATOM | 6233 | O | LEU | M | 154 | 67.869 | −9.244 | −33.830 | 1.00 | 25.58 | O |
| ATOM | 6234 | N | GLN | M | 155 | 67.478 | −9.457 | −31.616 | 1.00 | 25.28 | N |
| ATOM | 6235 | CA | GLN | M | 155 | 67.354 | −10.920 | −31.634 | 1.00 | 25.20 | C |
| ATOM | 6236 | CB | GLN | M | 155 | 67.876 | −11.524 | −30.317 | 1.00 | 25.25 | C |
| ATOM | 6237 | CG | GLN | M | 155 | 69.349 | −11.209 | −30.020 | 1.00 | 24.47 | C |
| ATOM | 6238 | CD | GLN | M | 155 | 70.275 | −11.727 | −31.102 | 1.00 | 24.76 | C |
| ATOM | 6239 | OE1 | GLN | M | 155 | 70.474 | −12.936 | −31.232 | 1.00 | 24.38 | O |
| ATOM | 6240 | NE2 | GLN | M | 155 | 70.839 | −10.815 | −31.890 | 1.00 | 23.50 | N |
| ATOM | 6241 | C | GLN | M | 155 | 65.910 | −11.352 | −31.844 | 1.00 | 25.33 | C |
| ATOM | 6242 | O | GLN | M | 155 | 64.984 | −10.656 | −31.433 | 1.00 | 25.28 | O |
| ATOM | 6243 | N | SER | M | 156 | 65.722 | −12.506 | −32.477 | 1.00 | 25.61 | N |
| ATOM | 6244 | CA | SER | M | 156 | 64.384 | −13.079 | −32.649 | 1.00 | 25.75 | C |
| ATOM | 6245 | CB | SER | M | 156 | 63.768 | −12.656 | −33.988 | 1.00 | 25.82 | C |
| ATOM | 6246 | OG | SER | M | 156 | 64.025 | −11.293 | −34.258 | 1.00 | 27.14 | O |
| ATOM | 6247 | C | SER | M | 156 | 64.431 | −14.590 | −32.602 | 1.00 | 25.42 | C |
| ATOM | 6248 | O | SER | M | 156 | 65.346 | −15.207 | −33.160 | 1.00 | 25.56 | O |
| ATOM | 6249 | N | GLY | M | 157 | 63.434 | −15.178 | −31.948 | 1.00 | 25.23 | N |
| ATOM | 6250 | CA | GLY | M | 157 | 63.235 | −16.620 | −31.991 | 1.00 | 24.54 | C |
| ATOM | 6251 | C | GLY | M | 157 | 64.144 | −17.415 | −31.073 | 1.00 | 24.02 | C |
| ATOM | 6252 | O | GLY | M | 157 | 63.948 | −18.614 | −30.923 | 1.00 | 24.17 | O |
| ATOM | 6253 | N | ASN | M | 158 | 65.126 | −16.754 | −30.455 | 1.00 | 23.31 | N |
| ATOM | 6254 | CA | ASN | M | 158 | 66.092 | −17.438 | −29.573 | 1.00 | 22.30 | C |
| ATOM | 6255 | CB | ASN | M | 158 | 67.545 | −17.228 | −30.053 | 1.00 | 22.45 | C |
| ATOM | 6256 | CG | ASN | M | 158 | 67.947 | −15.758 | −30.143 | 1.00 | 22.23 | C |
| ATOM | 6257 | OD1 | ASN | M | 158 | 67.191 | −14.854 | −29.776 | 1.00 | 23.36 | O |
| ATOM | 6258 | ND2 | ASN | M | 158 | 69.164 | −15.518 | −30.622 | 1.00 | 22.37 | N |
| ATOM | 6259 | C | ASN | M | 158 | 65.934 | −17.111 | −28.082 | 1.00 | 21.72 | C |
| ATOM | 6260 | O | ASN | M | 158 | 66.862 | −17.309 | −27.284 | 1.00 | 21.09 | O |
| ATOM | 6261 | N | SER | M | 159 | 64.752 | −16.614 | −27.724 | 1.00 | 20.82 | N |
| ATOM | 6262 | CA | SER | M | 159 | 64.433 | −16.270 | −26.341 | 1.00 | 20.62 | C |
| ATOM | 6263 | CB | SER | M | 159 | 64.495 | −14.749 | −26.133 | 1.00 | 20.77 | C |
| ATOM | 6264 | OG | SER | M | 159 | 63.501 | −14.081 | −26.886 | 1.00 | 19.19 | O |
| ATOM | 6265 | C | SER | M | 159 | 63.079 | −16.826 | −25.911 | 1.00 | 20.50 | C |
| ATOM | 6266 | O | SER | M | 159 | 62.157 | −16.984 | −26.720 | 1.00 | 20.05 | O |
| ATOM | 6267 | N | GLN | M | 160 | 62.963 | −17.150 | −24.629 | 1.00 | 20.70 | N |
| ATOM | 6268 | CA | GLN | M | 160 | 61.701 | −17.603 | −24.075 | 1.00 | 20.53 | C |
| ATOM | 6269 | CB | GLN | M | 160 | 61.736 | −19.108 | −23.800 | 1.00 | 20.48 | C |
| ATOM | 6270 | CG | GLN | M | 160 | 61.724 | −19.965 | −25.068 | 1.00 | 20.83 | C |
| ATOM | 6271 | CD | GLN | M | 160 | 61.586 | −21.442 | −24.759 | 1.00 | 20.85 | C |
| ATOM | 6272 | OE1 | GLN | M | 160 | 62.560 | −22.107 | −24.396 | 1.00 | 21.60 | O |
| ATOM | 6273 | NE2 | GLN | M | 160 | 60.383 | −21.965 | −24.909 | 1.00 | 18.79 | N |
| ATOM | 6274 | C | GLN | M | 160 | 61.410 | −16.821 | −22.805 | 1.00 | 21.03 | C |
| ATOM | 6275 | O | GLN | M | 160 | 62.340 | −16.474 | −22.065 | 1.00 | 20.31 | O |
| ATOM | 6276 | N | GLU | M | 161 | 60.118 | −16.557 | −22.585 | 1.00 | 21.38 | N |
| ATOM | 6277 | CA | GLU | M | 161 | 59.581 | −15.716 | −21.499 | 1.00 | 22.40 | C |
| ATOM | 6278 | CB | GLU | M | 161 | 58.669 | −14.624 | −22.072 | 1.00 | 22.40 | C |
| ATOM | 6279 | CG | GLU | M | 161 | 59.359 | −13.418 | −22.593 | 1.00 | 26.21 | C |
| ATOM | 6280 | CD | GLU | M | 161 | 58.426 | −12.229 | −22.707 | 1.00 | 27.45 | C |
| ATOM | 6281 | OE1 | GLU | M | 161 | 58.945 | −11.123 | −22.929 | 1.00 | 30.61 | O |
| ATOM | 6282 | OE2 | GLU | M | 161 | 57.185 | −12.392 | −22.576 | 1.00 | 28.70 | O |
| ATOM | 6283 | C | GLU | M | 161 | 58.685 | −16.516 | −20.576 | 1.00 | 22.05 | C |
| ATOM | 6284 | O | GLU | M | 161 | 57.941 | −17.401 | −21.015 | 1.00 | 21.89 | O |
| ATOM | 6285 | N | SER | M | 162 | 58.693 | −16.126 | −19.309 | 1.00 | 21.84 | N |
| ATOM | 6286 | CA | SER | M | 162 | 57.862 | −16.736 | −18.300 | 1.00 | 22.28 | C |
| ATOM | 6287 | CB | SER | M | 162 | 58.691 | −17.830 | −17.643 | 1.00 | 22.45 | C |
| ATOM | 6288 | OG | SER | M | 162 | 57.986 | −18.457 | −16.633 | 1.00 | 21.46 | O |
| ATOM | 6289 | C | SER | M | 162 | 57.437 | −15.659 | −17.284 | 1.00 | 22.27 | C |
| ATOM | 6290 | O | SER | M | 162 | 58.260 | −14.825 | −16.894 | 1.00 | 21.93 | O |
| ATOM | 6291 | N | VAL | M | 163 | 56.170 | −15.686 | −16.852 | 1.00 | 22.58 | N |
| ATOM | 6292 | CA | VAL | M | 163 | 55.621 | −14.654 | −15.936 | 1.00 | 23.35 | C |
| ATOM | 6293 | CB | VAL | M | 163 | 54.709 | −13.635 | −16.712 | 1.00 | 23.74 | C |
| ATOM | 6294 | CG1 | VAL | M | 163 | 53.542 | −14.346 | −17.401 | 1.00 | 25.35 | C |
| ATOM | 6295 | CG2 | VAL | M | 163 | 54.178 | −12.526 | −15.802 | 1.00 | 24.23 | C |
| ATOM | 6296 | C | VAL | M | 163 | 54.918 | −15.253 | −14.694 | 1.00 | 23.51 | C |
| ATOM | 6297 | O | VAL | M | 163 | 54.218 | −16.268 | −14.794 | 1.00 | 23.19 | O |
| ATOM | 6298 | N | THR | M | 164 | 55.144 | −14.652 | −13.524 | 1.00 | 23.30 | N |
| ATOM | 6299 | CA | THR | M | 164 | 54.465 | −15.077 | −12.299 | 1.00 | 23.73 | C |
| ATOM | 6300 | CB | THR | M | 164 | 55.083 | −14.459 | −11.036 | 1.00 | 24.35 | C |
| ATOM | 6301 | OG1 | THR | M | 164 | 55.087 | −13.025 | −11.150 | 1.00 | 25.01 | O |
| ATOM | 6302 | CG2 | THR | M | 164 | 56.510 | −14.959 | −10.826 | 1.00 | 24.82 | C |
| ATOM | 6303 | C | THR | M | 164 | 52.993 | −14.689 | −12.326 | 1.00 | 23.96 | C |
| ATOM | 6304 | O | THR | M | 164 | 52.578 | −13.835 | −13.107 | 1.00 | 24.18 | O |
| ATOM | 6305 | N | GLU | M | 165 | 52.205 | −15.342 | −11.475 | 1.00 | 23.98 | N |
| ATOM | 6306 | CA | GLU | M | 165 | 50.848 | −14.901 | −11.212 | 1.00 | 23.84 | C |
| ATOM | 6307 | CB | GLU | M | 165 | 50.006 | −16.045 | −10.619 | 1.00 | 24.20 | C |
| ATOM | 6308 | CG | GLU | M | 165 | 49.648 | −17.128 | −11.665 | 1.00 | 26.14 | C |
| ATOM | 6309 | CD | GLU | M | 165 | 49.043 | −16.555 | −12.952 | 1.00 | 29.34 | C |
| ATOM | 6310 | OE1 | GLU | M | 165 | 47.942 | −15.968 | −12.887 | 1.00 | 29.72 | O |
| ATOM | 6311 | OE2 | GLU | M | 165 | 49.665 | −16.682 | −14.040 | 1.00 | 28.89 | O |
| ATOM | 6312 | C | GLU | M | 165 | 50.916 | −13.715 | −10.260 | 1.00 | 22.99 | C |
| ATOM | 6313 | O | GLU | M | 165 | 51.923 | −13.499 | −9.571 | 1.00 | 21.91 | O |
| ATOM | 6314 | N | GLN | M | 166 | 49.837 | −12.951 | −10.229 | 1.00 | 22.29 | N |
| ATOM | 6315 | CA | GLN | M | 166 | 49.808 | −11.718 | −9.479 | 1.00 | 22.37 | C |
| ATOM | 6316 | CB | GLN | M | 166 | 48.427 | −11.079 | −9.632 | 1.00 | 22.39 | C |
| ATOM | 6317 | CG | GLN | M | 166 | 48.398 | −9.623 | −9.249 | 1.00 | 22.79 | C |
| ATOM | 6318 | CD | GLN | M | 166 | 47.044 | −8.996 | −9.445 | 1.00 | 22.71 | C |
| ATOM | 6319 | OE1 | GLN | M | 166 | 46.016 | −9.659 | −9.308 | 1.00 | 24.20 | O |
| ATOM | 6320 | NE2 | GLN | M | 166 | 47.030 | −7.705 | −9.753 | 1.00 | 21.80 | N |
| ATOM | 6321 | C | GLN | M | 166 | 50.177 | −11.937 | −7.999 | 1.00 | 22.21 | C |
| ATOM | 6322 | O | GLN | M | 166 | 49.682 | −12.862 | −7.348 | 1.00 | 20.64 | O |
| ATOM | 6323 | N | ASP | M | 167 | 51.052 | −11.086 | −7.465 | 1.00 | 22.29 | N |
| ATOM | 6324 | CA | ASP | M | 167 | 51.490 | −11.224 | −6.070 | 1.00 | 23.47 | C |
| ATOM | 6325 | CB | ASP | M | 167 | 52.725 | −10.354 | −5.788 | 1.00 | 23.28 | C |
| ATOM | 6326 | CG | ASP | M | 167 | 53.331 | −10.612 | −4.408 | 1.00 | 26.95 | C |
| ATOM | 6327 | OD1 | ASP | M | 167 | 52.903 | −9.966 | −3.422 | 1.00 | 28.32 | O |
| ATOM | 6328 | OD2 | ASP | M | 167 | 54.262 | −11.446 | −4.310 | 1.00 | 29.82 | O |
| ATOM | 6329 | C | ASP | M | 167 | 50.331 | −10.901 | −5.111 | 1.00 | 24.03 | C |
| ATOM | 6330 | O | ASP | M | 167 | 49.626 | −9.903 | −5.278 | 1.00 | 22.82 | O |
| ATOM | 6331 | N | SER | M | 168 | 50.133 | −11.764 | −4.117 | 1.00 | 24.59 | N |
| ATOM | 6332 | CA | SER | M | 168 | 48.982 | −11.656 | −3.228 | 1.00 | 25.98 | C |
| ATOM | 6333 | CB | SER | M | 168 | 48.878 | −12.916 | −2.367 | 1.00 | 26.11 | C |
| ATOM | 6334 | OG | SER | M | 168 | 49.968 | −12.956 | −1.464 | 1.00 | 27.62 | O |
| ATOM | 6335 | C | SER | M | 168 | 49.056 | −10.390 | −2.341 | 1.00 | 26.28 | C |
| ATOM | 6336 | O | SER | M | 168 | 48.030 | −9.915 | −1.846 | 1.00 | 27.34 | O |
| ATOM | 6337 | N | LYS | M | 169 | 50.253 | −9.837 | −2.168 | 1.00 | 25.92 | N |
| ATOM | 6338 | CA | LYS | M | 169 | 50.446 | −8.716 | −1.245 | 1.00 | 26.07 | C |
| ATOM | 6339 | CB | LYS | M | 169 | 51.676 | −8.928 | −0.358 | 1.00 | 26.95 | C |
| ATOM | 6340 | CG | LYS | M | 169 | 51.449 | −9.792 | 0.884 | 1.00 | 29.79 | C |
| ATOM | 6341 | CD | LYS | M | 169 | 52.729 | −9.790 | 1.741 | 1.00 | 33.28 | C |
| ATOM | 6342 | CE | LYS | M | 169 | 52.715 | −10.836 | 2.859 | 1.00 | 35.75 | C |
| ATOM | 6343 | NZ | LYS | M | 169 | 52.040 | −12.129 | 2.485 | 1.00 | 37.89 | N |
| ATOM | 6344 | C | LYS | M | 169 | 50.543 | −7.350 | −1.921 | 1.00 | 24.80 | C |
| ATOM | 6345 | O | LYS | M | 169 | 49.915 | −6.401 | −1.469 | 1.00 | 25.26 | O |
| ATOM | 6346 | N | ASP | M | 170 | 51.353 | −7.241 | −2.968 | 1.00 | 23.43 | N |
| ATOM | 6347 | CA | ASP | M | 170 | 51.502 | −5.971 | −3.697 | 1.00 | 21.73 | C |
| ATOM | 6348 | CB | ASP | M | 170 | 52.976 | −5.550 | −3.800 | 1.00 | 21.86 | C |
| ATOM | 6349 | CG | ASP | M | 170 | 53.826 | −6.534 | −4.600 | 1.00 | 22.15 | C |
| ATOM | 6350 | OD1 | ASP | M | 170 | 53.328 | −7.172 | −5.550 | 1.00 | 20.82 | O |
| ATOM | 6351 | OD2 | ASP | M | 170 | 55.029 | −6.645 | −4.293 | 1.00 | 25.23 | O |
| ATOM | 6352 | C | ASP | M | 170 | 50.816 | −5.957 | −5.074 | 1.00 | 20.25 | C |
| ATOM | 6353 | O | ASP | M | 170 | 50.856 | −4.953 | −5.778 | 1.00 | 18.82 | O |
| ATOM | 6354 | N | SER | M | 171 | 50.188 | −7.073 | −5.451 | 1.00 | 19.00 | N |
| ATOM | 6355 | CA | SER | M | 171 | 49.438 | −7.174 | −6.715 | 1.00 | 18.41 | C |
| ATOM | 6356 | CB | SER | M | 171 | 48.237 | −6.221 | −6.685 | 1.00 | 18.15 | C |
| ATOM | 6357 | OG | SER | M | 171 | 47.347 | −6.568 | −5.636 | 1.00 | 19.04 | O |
| ATOM | 6358 | C | SER | M | 171 | 50.262 | −6.956 | −8.009 | 1.00 | 17.88 | C |
| ATOM | 6359 | O | SER | M | 171 | 49.712 | −6.594 | −9.054 | 1.00 | 17.91 | O |
| ATOM | 6360 | N | THR | M | 172 | 51.564 | −7.195 | −7.942 | 1.00 | 17.44 | N |
| ATOM | 6361 | CA | THR | M | 172 | 52.429 | −7.041 | −9.105 | 1.00 | 17.14 | C |
| ATOM | 6362 | CB | THR | M | 172 | 53.789 | −6.406 | −8.723 | 1.00 | 17.51 | C |
| ATOM | 6363 | OG1 | THR | M | 172 | 54.450 | −7.241 | −7.764 | 1.00 | 17.92 | O |
| ATOM | 6364 | CG2 | THR | M | 172 | 53.602 | −5.008 | −8.137 | 1.00 | 16.91 | C |
| ATOM | 6365 | C | THR | M | 172 | 52.717 | −8.378 | −9.782 | 1.00 | 17.66 | C |
| ATOM | 6366 | O | THR | M | 172 | 52.394 | −9.451 | −9.238 | 1.00 | 17.08 | O |
| ATOM | 6367 | N | TYR | M | 173 | 53.342 | −8.281 | −10.950 | 1.00 | 17.25 | N |
| ATOM | 6368 | CA | TYR | M | 173 | 53.879 | −9.407 | −11.696 | 1.00 | 18.50 | C |
| ATOM | 6369 | CB | TYR | M | 173 | 53.326 | −9.404 | −13.132 | 1.00 | 18.17 | C |
| ATOM | 6370 | CG | TYR | M | 173 | 51.828 | −9.439 | −13.170 | 1.00 | 20.09 | C |
| ATOM | 6371 | CD1 | TYR | M | 173 | 51.090 | −8.254 | −13.287 | 1.00 | 19.53 | C |
| ATOM | 6372 | CE1 | TYR | M | 173 | 49.714 | −8.265 | −13.293 | 1.00 | 20.98 | C |
| ATOM | 6373 | CZ | TYR | M | 173 | 49.045 | −9.476 | −13.170 | 1.00 | 22.87 | C |
| ATOM | 6374 | OH | TYR | M | 173 | 47.674 | −9.483 | −13.179 | 1.00 | 25.79 | O |
| ATOM | 6375 | CE2 | TYR | M | 173 | 49.745 | −10.670 | −13.046 | 1.00 | 21.85 | C |
| ATOM | 6376 | CD2 | TYR | M | 173 | 51.137 | −10.646 | −13.036 | 1.00 | 20.95 | C |
| ATOM | 6377 | C | TYR | M | 173 | 55.393 | −9.305 | −11.776 | 1.00 | 18.52 | C |
| ATOM | 6378 | O | TYR | M | 173 | 55.962 | −8.213 | −11.656 | 1.00 | 17.67 | O |
| ATOM | 6379 | N | SER | M | 174 | 56.038 | −10.451 | −11.972 | 1.00 | 18.50 | N |
| ATOM | 6380 | CA | SER | M | 174 | 57.426 | −10.474 | −12.386 | 1.00 | 18.35 | C |
| ATOM | 6381 | CB | SER | M | 174 | 58.332 | −10.933 | −11.246 | 1.00 | 18.90 | C |
| ATOM | 6382 | OG | SER | M | 174 | 58.307 | −10.012 | −10.153 | 1.00 | 18.93 | O |
| ATOM | 6383 | C | SER | M | 174 | 57.558 | −11.385 | −13.596 | 1.00 | 18.73 | C |
| ATOM | 6384 | O | SER | M | 174 | 56.751 | −12.307 | −13.781 | 1.00 | 18.68 | O |
| ATOM | 6385 | N | LEU | M | 175 | 58.583 | −11.121 | −14.409 | 1.00 | 18.47 | N |
| ATOM | 6386 | CA | LEU | M | 175 | 58.807 | −11.823 | −15.659 | 1.00 | 18.85 | C |
| ATOM | 6387 | CB | LEU | M | 175 | 58.303 | −10.976 | −16.831 | 1.00 | 18.75 | C |
| ATOM | 6388 | CG | LEU | M | 175 | 58.411 | −11.463 | −18.284 | 1.00 | 20.12 | C |
| ATOM | 6389 | CD1 | LEU | M | 175 | 57.395 | −10.788 | −19.206 | 1.00 | 21.41 | C |
| ATOM | 6390 | CD2 | LEU | M | 175 | 59.797 | −11.280 | −18.856 | 1.00 | 19.01 | C |
| ATOM | 6391 | C | LEU | M | 175 | 60.299 | −12.134 | −15.827 | 1.00 | 19.05 | C |
| ATOM | 6392 | O | LEU | M | 175 | 61.150 | −11.312 | −15.499 | 1.00 | 16.99 | O |
| ATOM | 6393 | N | SER | M | 176 | 60.606 | −13.322 | −16.351 | 1.00 | 19.98 | N |
| ATOM | 6394 | CA | SER | M | 176 | 61.969 | −13.638 | −16.738 | 1.00 | 21.73 | C |
| ATOM | 6395 | CB | SER | M | 176 | 62.504 | −14.819 | −15.935 | 1.00 | 22.26 | C |
| ATOM | 6396 | OG | SER | M | 176 | 61.815 | −16.002 | −16.310 | 1.00 | 27.31 | O |
| ATOM | 6397 | C | SER | M | 176 | 62.012 | −13.960 | −18.215 | 1.00 | 21.53 | C |
| ATOM | 6398 | O | SER | M | 176 | 61.127 | −14.648 | −18.729 | 1.00 | 22.07 | O |
| ATOM | 6399 | N | SER | M | 177 | 63.029 | −13.438 | −18.892 | 1.00 | 20.85 | N |
| ATOM | 6400 | CA | SER | M | 177 | 63.303 | −13.780 | −20.279 | 1.00 | 20.73 | C |
| ATOM | 6401 | CB | SER | M | 177 | 63.225 | −12.552 | −21.178 | 1.00 | 20.56 | C |
| ATOM | 6402 | OG | SER | M | 177 | 63.411 | −12.928 | −22.531 | 1.00 | 22.67 | O |
| ATOM | 6403 | C | SER | M | 177 | 64.683 | −14.418 | −20.363 | 1.00 | 19.87 | C |
| ATOM | 6404 | O | SER | M | 177 | 65.635 | −13.932 | −19.749 | 1.00 | 19.51 | O |
| ATOM | 6405 | N | THR | M | 178 | 64.774 | −15.528 | −21.089 | 1.00 | 18.88 | N |
| ATOM | 6406 | CA | THR | M | 178 | 66.053 | −16.198 | −21.271 | 1.00 | 18.48 | C |
| ATOM | 6407 | CB | THR | M | 178 | 66.065 | −17.620 | −20.686 | 1.00 | 18.46 | C |
| ATOM | 6408 | OG1 | THR | M | 178 | 65.721 | −17.564 | −19.296 | 1.00 | 19.02 | O |
| ATOM | 6409 | CG2 | THR | M | 178 | 67.444 | −18.247 | −20.820 | 1.00 | 18.83 | C |
| ATOM | 6410 | C | THR | M | 178 | 66.421 | −16.235 | −22.741 | 1.00 | 18.02 | C |
| ATOM | 6411 | O | THR | M | 178 | 65.643 | −16.696 | −23.569 | 1.00 | 17.83 | O |
| ATOM | 6412 | N | LEU | M | 179 | 67.605 | −15.723 | −23.044 | 1.00 | 17.34 | N |
| ATOM | 6413 | CA | LEU | M | 179 | 68.193 | −15.851 | −24.375 | 1.00 | 17.41 | C |
| ATOM | 6414 | CB | LEU | M | 179 | 68.881 | −14.546 | −24.782 | 1.00 | 16.78 | C |
| ATOM | 6415 | CG | LEU | M | 179 | 69.693 | −14.481 | −26.083 | 1.00 | 17.03 | C |
| ATOM | 6416 | CD1 | LEU | M | 179 | 68.754 | −14.261 | −27.255 | 1.00 | 15.96 | C |
| ATOM | 6417 | CD2 | LEU | M | 179 | 70.700 | −13.350 | −25.993 | 1.00 | 16.10 | C |
| ATOM | 6418 | C | LEU | M | 179 | 69.198 | −16.994 | −24.332 | 1.00 | 17.32 | C |
| ATOM | 6419 | O | LEU | M | 179 | 70.094 | −17.037 | −23.465 | 1.00 | 17.19 | O |
| ATOM | 6420 | N | THR | M | 180 | 69.039 | −17.924 | −25.264 | 1.00 | 17.56 | N |
| ATOM | 6421 | CA | THR | M | 180 | 69.923 | −19.076 | −25.355 | 1.00 | 17.65 | C |
| ATOM | 6422 | CB | THR | M | 180 | 69.127 | −20.404 | −25.272 | 1.00 | 18.03 | C |
| ATOM | 6423 | OG1 | THR | M | 180 | 68.229 | −20.364 | −24.156 | 1.00 | 17.60 | O |
| ATOM | 6424 | CG2 | THR | M | 180 | 70.075 | −21.599 | −25.102 | 1.00 | 18.08 | C |
| ATOM | 6425 | C | THR | M | 180 | 70.712 | −19.002 | −26.655 | 1.00 | 18.03 | C |
| ATOM | 6426 | O | THR | M | 180 | 70.130 | −18.842 | −27.731 | 1.00 | 17.53 | O |
| ATOM | 6427 | N | LEU | M | 181 | 72.035 | −19.099 | −26.536 | 1.00 | 17.81 | N |
| ATOM | 6428 | CA | LEU | M | 181 | 72.929 | −19.161 | −27.677 | 1.00 | 18.75 | C |
| ATOM | 6429 | CB | LEU | M | 181 | 73.760 | −17.876 | −27.777 | 1.00 | 18.97 | C |
| ATOM | 6430 | CG | LEU | M | 181 | 73.058 | −16.535 | −28.012 | 1.00 | 20.69 | C |
| ATOM | 6431 | CD1 | LEU | M | 181 | 74.012 | −15.393 | −27.693 | 1.00 | 22.68 | C |
| ATOM | 6432 | CD2 | LEU | M | 181 | 72.554 | −16.415 | −29.451 | 1.00 | 22.28 | C |
| ATOM | 6433 | C | LEU | M | 181 | 73.870 | −20.341 | −27.501 | 1.00 | 18.63 | C |
| ATOM | 6434 | O | LEU | M | 181 | 74.127 | −20.766 | −26.372 | 1.00 | 18.45 | O |
| ATOM | 6435 | N | SER | M | 182 | 74.384 | −20.873 | −28.607 | 1.00 | 18.61 | N |
| ATOM | 6436 | CA | SER | M | 182 | 75.475 | −21.823 | −28.529 | 1.00 | 18.95 | C |
| ATOM | 6437 | CB | SER | M | 182 | 75.823 | −22.388 | −29.913 | 1.00 | 18.95 | C |
| ATOM | 6438 | OG | SER | M | 182 | 76.480 | −21.412 | −30.710 | 1.00 | 18.64 | O |
| ATOM | 6439 | C | SER | M | 182 | 76.673 | −21.084 | −27.930 | 1.00 | 19.42 | C |
| ATOM | 6440 | O | SER | M | 182 | 76.772 | −19.854 | −28.036 | 1.00 | 18.80 | O |
| ATOM | 6441 | N | LYS | M | 183 | 77.565 | −21.833 | −27.289 | 1.00 | 20.24 | N |
| ATOM | 6442 | CA | LYS | M | 183 | 78.777 | −21.260 | −26.707 | 1.00 | 21.20 | C |
| ATOM | 6443 | CB | LYS | M | 183 | 79.629 | −22.379 | −26.108 | 1.00 | 21.54 | C |
| ATOM | 6444 | CG | LYS | M | 183 | 80.817 | −21.930 | −25.286 | 1.00 | 22.77 | C |
| ATOM | 6445 | CD | LYS | M | 183 | 81.804 | −23.077 | −25.136 | 1.00 | 23.88 | C |
| ATOM | 6446 | CE | LYS | M | 183 | 83.106 | −22.610 | −24.523 | 1.00 | 24.64 | C |
| ATOM | 6447 | NZ | LYS | M | 183 | 84.051 | −23.741 | −24.339 | 1.00 | 25.93 | N |
| ATOM | 6448 | C | LYS | M | 183 | 79.564 | −20.463 | −27.764 | 1.00 | 21.46 | C |
| ATOM | 6449 | O | LYS | M | 183 | 80.005 | −19.337 | −27.500 | 1.00 | 21.81 | O |
| ATOM | 6450 | N | ALA | M | 184 | 79.702 | −21.049 | −28.956 | 1.00 | 21.45 | N |
| ATOM | 6451 | CA | ALA | M | 184 | 80.384 | −20.420 | −30.089 | 1.00 | 21.74 | C |
| ATOM | 6452 | CB | ALA | M | 184 | 80.379 | −21.355 | −31.300 | 1.00 | 21.71 | C |
| ATOM | 6453 | C | ALA | M | 184 | 79.815 | −19.044 | −30.463 | 1.00 | 21.74 | C |
| ATOM | 6454 | O | ALA | M | 184 | 80.577 | −18.095 | −30.667 | 1.00 | 21.54 | O |
| ATOM | 6455 | N | ASP | M | 185 | 78.487 | −18.937 | −30.543 | 1.00 | 21.86 | N |
| ATOM | 6456 | CA | ASP | M | 185 | 77.838 | −17.672 | −30.907 | 1.00 | 22.10 | C |
| ATOM | 6457 | CB | ASP | M | 185 | 76.347 | −17.874 | −31.228 | 1.00 | 22.03 | C |
| ATOM | 6458 | CG | ASP | M | 185 | 76.111 | −18.437 | −32.623 | 1.00 | 22.39 | C |
| ATOM | 6459 | OD1 | ASP | M | 185 | 77.073 | −18.563 | −33.406 | 1.00 | 22.22 | O |
| ATOM | 6460 | OD2 | ASP | M | 185 | 74.949 | −18.751 | −32.946 | 1.00 | 22.37 | O |
| ATOM | 6461 | C | ASP | M | 185 | 78.002 | −16.625 | −29.812 | 1.00 | 22.28 | C |
| ATOM | 6462 | O | ASP | M | 185 | 78.230 | −15.444 | −30.093 | 1.00 | 22.10 | O |
| ATOM | 6463 | N | TYR | M | 186 | 77.877 | −17.069 | −28.562 | 1.00 | 22.64 | N |
| ATOM | 6464 | CA | TYR | M | 186 | 78.044 | −16.191 | −27.414 | 1.00 | 22.53 | C |
| ATOM | 6465 | CB | TYR | M | 186 | 77.804 | −16.943 | −26.096 | 1.00 | 22.23 | C |
| ATOM | 6466 | CG | TYR | M | 186 | 78.013 | −16.082 | −24.872 | 1.00 | 20.90 | C |
| ATOM | 6467 | CD1 | TYR | M | 186 | 77.112 | −15.072 | −24.553 | 1.00 | 20.97 | C |
| ATOM | 6468 | CE1 | TYR | M | 186 | 77.294 | −14.272 | −23.435 | 1.00 | 20.94 | C |
| ATOM | 6469 | CZ | TYR | M | 186 | 78.392 | −14.481 | −22.617 | 1.00 | 21.49 | C |
| ATOM | 6470 | OH | TYR | M | 186 | 78.566 | −13.684 | −21.511 | 1.00 | 22.17 | O |
| ATOM | 6471 | CE2 | TYR | M | 186 | 79.304 | −15.485 | −22.905 | 1.00 | 21.07 | C |
| ATOM | 6472 | CD2 | TYR | M | 186 | 79.110 | −16.278 | −24.034 | 1.00 | 21.04 | C |
| ATOM | 6473 | C | TYR | M | 186 | 79.427 | −15.561 | −27.415 | 1.00 | 22.93 | C |
| ATOM | 6474 | O | TYR | M | 186 | 79.574 | −14.373 | −27.125 | 1.00 | 22.94 | O |
| ATOM | 6475 | N | GLU | M | 187 | 80.431 | −16.361 | −27.764 | 1.00 | 23.67 | N |
| ATOM | 6476 | CA | GLU | M | 187 | 81.815 | −15.915 | −27.746 | 1.00 | 24.20 | C |
| ATOM | 6477 | CB | GLU | M | 187 | 82.769 | −17.109 | −27.662 | 1.00 | 24.19 | C |
| ATOM | 6478 | CG | GLU | M | 187 | 82.944 | −17.602 | −26.236 | 1.00 | 25.89 | C |
| ATOM | 6479 | CD | GLU | M | 187 | 83.690 | −18.924 | −26.127 | 1.00 | 28.03 | C |
| ATOM | 6480 | OE1 | GLU | M | 187 | 84.222 | −19.420 | −27.149 | 1.00 | 29.05 | O |
| ATOM | 6481 | OE2 | GLU | M | 187 | 83.741 | −19.469 | −25.002 | 1.00 | 28.98 | O |
| ATOM | 6482 | C | GLU | M | 187 | 82.210 | −14.967 | −28.880 | 1.00 | 24.30 | C |
| ATOM | 6483 | O | GLU | M | 187 | 83.218 | −14.270 | −28.763 | 1.00 | 24.32 | O |
| ATOM | 6484 | N | LYS | M | 188 | 81.430 | −14.917 | −29.959 | 1.00 | 24.25 | N |
| ATOM | 6485 | CA | LYS | M | 188 | 81.782 | −14.012 | −31.058 | 1.00 | 24.61 | C |
| ATOM | 6486 | CB | LYS | M | 188 | 81.517 | −14.642 | −32.437 | 1.00 | 24.60 | C |
| ATOM | 6487 | CG | LYS | M | 188 | 80.058 | −14.818 | −32.828 | 1.00 | 26.19 | C |
| ATOM | 6488 | CD | LYS | M | 188 | 79.914 | −15.278 | −34.289 | 1.00 | 27.65 | C |
| ATOM | 6489 | CE | LYS | M | 188 | 80.052 | −14.117 | −35.279 | 1.00 | 28.14 | C |
| ATOM | 6490 | NZ | LYS | M | 188 | 79.855 | −14.559 | −36.691 | 1.00 | 28.62 | N |
| ATOM | 6491 | C | LYS | M | 188 | 81.190 | −12.591 | −30.928 | 1.00 | 24.53 | C |
| ATOM | 6492 | O | LYS | M | 188 | 81.408 | −11.744 | −31.796 | 1.00 | 24.39 | O |
| ATOM | 6493 | N | HIS | M | 189 | 80.472 | −12.331 | −29.835 | 1.00 | 24.26 | N |
| ATOM | 6494 | CA | HIS | M | 189 | 79.931 | −10.991 | −29.569 | 1.00 | 24.01 | C |
| ATOM | 6495 | CB | HIS | M | 189 | 78.419 | −10.981 | −29.764 | 1.00 | 24.21 | C |
| ATOM | 6496 | CG | HIS | M | 189 | 77.996 | −11.373 | −31.145 | 1.00 | 24.52 | C |
| ATOM | 6497 | ND1 | HIS | M | 189 | 78.193 | −10.561 | −32.240 | 1.00 | 24.81 | N |
| ATOM | 6498 | CE1 | HIS | M | 189 | 77.723 | −11.160 | −33.319 | 1.00 | 25.77 | C |
| ATOM | 6499 | NE2 | HIS | M | 189 | 77.234 | −12.335 | −32.964 | 1.00 | 25.58 | N |
| ATOM | 6500 | CD2 | HIS | M | 189 | 77.395 | −12.494 | −31.609 | 1.00 | 25.13 | C |
| ATOM | 6501 | C | HIS | M | 189 | 80.304 | −10.435 | −28.193 | 1.00 | 23.82 | C |
| ATOM | 6502 | O | HIS | M | 189 | 80.601 | −11.193 | −27.271 | 1.00 | 23.70 | O |
| ATOM | 6503 | N | LYS | M | 190 | 80.279 | −9.108 | −28.068 | 1.00 | 23.58 | N |
| ATOM | 6504 | CA | LYS | M | 190 | 80.696 | −8.417 | −26.842 | 1.00 | 23.61 | C |
| ATOM | 6505 | CB | LYS | M | 190 | 81.624 | −7.236 | −27.194 | 1.00 | 23.93 | C |
| ATOM | 6506 | CG | LYS | M | 190 | 82.319 | −6.559 | −25.994 | 1.00 | 25.00 | C |
| ATOM | 6507 | CD | LYS | M | 190 | 83.374 | −5.515 | −26.428 | 1.00 | 27.22 | C |
| ATOM | 6508 | CE | LYS | M | 190 | 84.621 | −6.169 | −27.058 | 1.00 | 28.00 | C |
| ATOM | 6509 | NZ | LYS | M | 190 | 85.707 | −5.200 | −27.455 | 1.00 | 28.95 | N |
| ATOM | 6510 | C | LYS | M | 190 | 79.520 | −7.945 | −25.965 | 1.00 | 23.28 | C |
| ATOM | 6511 | O | LYS | M | 190 | 79.450 | −8.275 | −24.769 | 1.00 | 22.90 | O |
| ATOM | 6512 | N | VAL | M | 191 | 78.605 | −7.179 | −26.562 | 1.00 | 22.83 | N |
| ATOM | 6513 | CA | VAL | M | 191 | 77.584 | −6.459 | −25.800 | 1.00 | 22.38 | C |
| ATOM | 6514 | CB | VAL | M | 191 | 77.416 | −4.983 | −26.273 | 1.00 | 22.44 | C |
| ATOM | 6515 | CG1 | VAL | M | 191 | 76.713 | −4.160 | −25.195 | 1.00 | 21.88 | C |
| ATOM | 6516 | CG2 | VAL | M | 191 | 78.771 | −4.352 | −26.615 | 1.00 | 22.84 | C |
| ATOM | 6517 | C | VAL | M | 191 | 76.224 | −7.169 | −25.813 | 1.00 | 22.14 | C |
| ATOM | 6518 | O | VAL | M | 191 | 75.621 | −7.376 | −26.868 | 1.00 | 21.72 | O |
| ATOM | 6519 | N | TYR | M | 192 | 75.765 | −7.534 | −24.617 | 1.00 | 21.87 | N |
| ATOM | 6520 | CA | TYR | M | 192 | 74.506 | −8.236 | −24.431 | 1.00 | 21.80 | C |
| ATOM | 6521 | CB | TYR | M | 192 | 74.732 | −9.576 | −23.707 | 1.00 | 22.05 | C |
| ATOM | 6522 | CG | TYR | M | 192 | 75.451 | −10.557 | −24.597 | 1.00 | 22.24 | C |
| ATOM | 6523 | CD1 | TYR | M | 192 | 76.844 | −10.531 | −24.713 | 1.00 | 20.93 | C |
| ATOM | 6524 | CE1 | TYR | M | 192 | 77.509 | −11.409 | −25.556 | 1.00 | 21.96 | C |
| ATOM | 6525 | CZ | TYR | M | 192 | 76.779 | −12.316 | −26.303 | 1.00 | 21.94 | C |
| ATOM | 6526 | OH | TYR | M | 192 | 77.431 | −13.176 | −27.143 | 1.00 | 21.64 | O |
| ATOM | 6527 | CE2 | TYR | M | 192 | 75.396 | −12.354 | −26.217 | 1.00 | 22.53 | C |
| ATOM | 6528 | CD2 | TYR | M | 192 | 74.739 | −11.476 | −25.363 | 1.00 | 21.65 | C |
| ATOM | 6529 | C | TYR | M | 192 | 73.593 | −7.329 | −23.637 | 1.00 | 21.80 | C |
| ATOM | 6530 | O | TYR | M | 192 | 73.943 | −6.911 | −22.536 | 1.00 | 21.83 | O |
| ATOM | 6531 | N | ALA | M | 193 | 72.442 | −7.004 | −24.216 | 1.00 | 21.48 | N |
| ATOM | 6532 | CA | ALA | M | 193 | 71.511 | −6.087 | −23.584 | 1.00 | 21.72 | C |
| ATOM | 6533 | CB | ALA | M | 193 | 71.606 | −4.710 | −24.217 | 1.00 | 21.85 | C |
| ATOM | 6534 | C | ALA | M | 193 | 70.066 | −6.585 | −23.589 | 1.00 | 21.85 | C |
| ATOM | 6535 | O | ALA | M | 193 | 69.590 | −7.189 | −24.552 | 1.00 | 21.52 | O |
| ATOM | 6536 | N | CYS | M | 194 | 69.387 | −6.324 | −22.479 | 1.00 | 21.93 | N |
| ATOM | 6537 | CA | CYS | M | 194 | 67.956 | −6.523 | −22.359 | 1.00 | 22.85 | C |
| ATOM | 6538 | CB | CYS | M | 194 | 67.644 | −7.213 | −21.021 | 1.00 | 22.42 | C |
| ATOM | 6539 | SG | CYS | M | 194 | 65.908 | −7.450 | −20.710 | 1.00 | 29.49 | S |
| ATOM | 6540 | C | CYS | M | 194 | 67.332 | −5.130 | −22.382 | 1.00 | 21.93 | C |
| ATOM | 6541 | O | CYS | M | 194 | 67.681 | −4.291 | −21.547 | 1.00 | 22.50 | O |
| ATOM | 6542 | N | GLU | M | 195 | 66.437 | −4.871 | −23.335 | 1.00 | 20.62 | N |
| ATOM | 6543 | CA | GLU | M | 195 | 65.721 | −3.600 | −23.364 | 1.00 | 19.65 | C |
| ATOM | 6544 | CB | GLU | M | 195 | 65.885 | −2.884 | −24.704 | 1.00 | 19.95 | C |
| ATOM | 6545 | CG | GLU | M | 195 | 64.990 | −1.657 | −24.831 | 1.00 | 21.45 | C |
| ATOM | 6546 | CD | GLU | M | 195 | 64.640 | −1.337 | −26.274 | 1.00 | 25.02 | C |
| ATOM | 6547 | OE1 | GLU | M | 195 | 63.722 | −1.995 | −26.827 | 1.00 | 26.19 | O |
| ATOM | 6548 | OE2 | GLU | M | 195 | 65.278 | −0.425 | −26.839 | 1.00 | 24.15 | O |
| ATOM | 6549 | C | GLU | M | 195 | 64.248 | −3.830 | −23.067 | 1.00 | 19.06 | C |
| ATOM | 6550 | O | GLU | M | 195 | 63.599 | −4.671 | −23.700 | 1.00 | 18.83 | O |
| ATOM | 6551 | N | VAL | M | 196 | 63.727 | −3.076 | −22.106 | 1.00 | 18.16 | N |
| ATOM | 6552 | CA | VAL | M | 196 | 62.402 | −3.347 | −21.531 | 1.00 | 17.53 | C |
| ATOM | 6553 | CB | VAL | M | 196 | 62.495 | −3.614 | −20.012 | 1.00 | 17.36 | C |
| ATOM | 6554 | CG1 | VAL | M | 196 | 61.100 | −3.653 | −19.371 | 1.00 | 17.77 | C |
| ATOM | 6555 | CG2 | VAL | M | 196 | 63.206 | −4.928 | −19.754 | 1.00 | 16.40 | C |
| ATOM | 6556 | C | VAL | M | 196 | 61.462 | −2.190 | −21.779 | 1.00 | 17.40 | C |
| ATOM | 6557 | O | VAL | M | 196 | 61.777 | −1.051 | −21.451 | 1.00 | 16.62 | O |
| ATOM | 6558 | N | THR | M | 197 | 60.307 | −2.494 | −22.356 | 1.00 | 17.58 | N |
| ATOM | 6559 | CA | THR | M | 197 | 59.266 | −1.492 | −22.570 | 1.00 | 17.92 | C |
| ATOM | 6560 | CB | THR | M | 197 | 58.827 | −1.470 | −24.043 | 1.00 | 18.13 | C |
| ATOM | 6561 | OG1 | THR | M | 197 | 59.975 | −1.208 | −24.857 | 1.00 | 19.02 | O |
| ATOM | 6562 | CG2 | THR | M | 197 | 57.782 | −0.380 | −24.294 | 1.00 | 19.57 | C |
| ATOM | 6563 | C | THR | M | 197 | 58.078 | −1.809 | −21.658 | 1.00 | 17.55 | C |
| ATOM | 6564 | O | THR | M | 197 | 57.588 | −2.947 | −21.642 | 1.00 | 16.76 | O |
| ATOM | 6565 | N | HIS | M | 198 | 57.635 | −0.809 | −20.885 | 1.00 | 17.26 | N |
| ATOM | 6566 | CA | HIS | M | 198 | 56.482 | −0.980 | −19.976 | 1.00 | 16.67 | C |
| ATOM | 6567 | CB | HIS | M | 198 | 56.912 | −1.628 | −18.648 | 1.00 | 16.47 | C |
| ATOM | 6568 | CG | HIS | M | 198 | 55.769 | −1.954 | −17.736 | 1.00 | 15.49 | C |
| ATOM | 6569 | ND1 | HIS | M | 198 | 55.285 | −1.061 | −16.800 | 1.00 | 15.50 | N |
| ATOM | 6570 | CE1 | HIS | M | 198 | 54.275 | −1.608 | −16.146 | 1.00 | 13.59 | C |
| ATOM | 6571 | NE2 | HIS | M | 198 | 54.070 | −2.820 | −16.637 | 1.00 | 15.43 | N |
| ATOM | 6572 | CD2 | HIS | M | 198 | 54.998 | −3.065 | −17.627 | 1.00 | 15.07 | C |
| ATOM | 6573 | C | HIS | M | 198 | 55.789 | 0.363 | −19.712 | 1.00 | 16.96 | C |
| ATOM | 6574 | O | HIS | M | 198 | 56.422 | 1.415 | −19.746 | 1.00 | 15.96 | O |
| ATOM | 6575 | N | GLN | M | 199 | 54.487 | 0.303 | −19.439 | 1.00 | 17.08 | N |
| ATOM | 6576 | CA | GLN | M | 199 | 53.686 | 1.501 | −19.160 | 1.00 | 17.56 | C |
| ATOM | 6577 | CB | GLN | M | 199 | 52.229 | 1.124 | −18.868 | 1.00 | 17.50 | C |
| ATOM | 6578 | CG | GLN | M | 199 | 51.313 | 2.348 | −18.879 | 1.00 | 18.22 | C |
| ATOM | 6579 | CD | GLN | M | 199 | 49.846 | 2.023 | −18.684 | 1.00 | 19.26 | C |
| ATOM | 6580 | OE1 | GLN | M | 199 | 49.449 | 0.864 | −18.598 | 1.00 | 17.00 | O |
| ATOM | 6581 | NE2 | GLN | M | 199 | 49.028 | 3.068 | −18.598 | 1.00 | 18.48 | N |
| ATOM | 6582 | C | GLN | M | 199 | 54.245 | 2.374 | −18.020 | 1.00 | 17.50 | C |
| ATOM | 6583 | O | GLN | M | 199 | 54.173 | 3.585 | −18.080 | 1.00 | 16.81 | O |
| ATOM | 6584 | N | GLY | M | 200 | 54.809 | 1.751 | −16.993 | 1.00 | 17.99 | N |
| ATOM | 6585 | CA | GLY | M | 200 | 55.392 | 2.505 | −15.867 | 1.00 | 18.26 | C |
| ATOM | 6586 | C | GLY | M | 200 | 56.706 | 3.215 | −16.157 | 1.00 | 18.39 | C |
| ATOM | 6587 | O | GLY | M | 200 | 57.201 | 3.950 | −15.316 | 1.00 | 17.98 | O |
| ATOM | 6588 | N | LEU | M | 201 | 57.269 | 2.987 | −17.345 | 1.00 | 19.15 | N |
| ATOM | 6589 | CA | LEU | M | 201 | 58.537 | 3.596 | −17.772 | 1.00 | 19.44 | C |
| ATOM | 6590 | CB | LEU | M | 201 | 59.472 | 2.519 | −18.343 | 1.00 | 19.41 | C |
| ATOM | 6591 | CG | LEU | M | 201 | 59.939 | 1.390 | −17.399 | 1.00 | 18.59 | C |
| ATOM | 6592 | CD1 | LEU | M | 201 | 60.626 | 0.250 | −18.162 | 1.00 | 17.72 | C |
| ATOM | 6593 | CD2 | LEU | M | 201 | 60.854 | 1.928 | −16.294 | 1.00 | 18.16 | C |
| ATOM | 6594 | C | LEU | M | 201 | 58.274 | 4.666 | −18.830 | 1.00 | 20.36 | C |
| ATOM | 6595 | O | LEU | M | 201 | 57.503 | 4.431 | −19.761 | 1.00 | 20.18 | O |
| ATOM | 6596 | N | SER | M | 202 | 58.906 | 5.835 | −18.692 | 1.00 | 21.14 | N |
| ATOM | 6597 | CA | SER | M | 202 | 58.686 | 6.937 | −19.639 | 1.00 | 22.48 | C |
| ATOM | 6598 | CB | SER | M | 202 | 59.217 | 8.275 | −19.095 | 1.00 | 22.24 | C |
| ATOM | 6599 | OG | SER | M | 202 | 60.609 | 8.205 | −18.853 | 1.00 | 22.92 | O |
| ATOM | 6600 | C | SER | M | 202 | 59.319 | 6.631 | −20.998 | 1.00 | 23.02 | C |
| ATOM | 6601 | O | SER | M | 202 | 58.826 | 7.069 | −22.037 | 1.00 | 22.94 | O |
| ATOM | 6602 | N | SER | M | 203 | 60.424 | 5.893 | −20.968 | 1.00 | 23.56 | N |
| ATOM | 6603 | CA | SER | M | 203 | 61.044 | 5.360 | −22.180 | 1.00 | 24.31 | C |
| ATOM | 6604 | CB | SER | M | 203 | 62.064 | 6.349 | −22.765 | 1.00 | 24.35 | C |
| ATOM | 6605 | OG | SER | M | 203 | 63.117 | 6.579 | −21.860 | 1.00 | 25.04 | O |
| ATOM | 6606 | C | SER | M | 203 | 61.669 | 4.005 | −21.827 | 1.00 | 24.39 | C |
| ATOM | 6607 | O | SER | M | 203 | 61.704 | 3.636 | −20.645 | 1.00 | 23.85 | O |
| ATOM | 6608 | N | PRO | M | 204 | 62.128 | 3.237 | −22.843 | 1.00 | 24.53 | N |
| ATOM | 6609 | CA | PRO | M | 204 | 62.588 | 1.890 | −22.492 | 1.00 | 24.24 | C |
| ATOM | 6610 | CB | PRO | M | 204 | 62.763 | 1.215 | −23.857 | 1.00 | 24.48 | C |
| ATOM | 6611 | CG | PRO | M | 204 | 61.848 | 1.989 | −24.776 | 1.00 | 24.45 | C |
| ATOM | 6612 | CD | PRO | M | 204 | 61.985 | 3.400 | −24.303 | 1.00 | 24.55 | C |
| ATOM | 6613 | C | PRO | M | 204 | 63.894 | 1.899 | −21.681 | 1.00 | 24.17 | C |
| ATOM | 6614 | O | PRO | M | 204 | 64.760 | 2.744 | −21.903 | 1.00 | 22.95 | O |
| ATOM | 6615 | N | VAL | M | 205 | 63.978 | 0.989 | −20.711 | 1.00 | 23.44 | N |
| ATOM | 6616 | CA | VAL | M | 205 | 65.151 | 0.811 | −19.860 | 1.00 | 23.61 | C |
| ATOM | 6617 | CB | VAL | M | 205 | 64.731 | 0.440 | −18.399 | 1.00 | 23.88 | C |
| ATOM | 6618 | CG1 | VAL | M | 205 | 65.890 | −0.126 | −17.587 | 1.00 | 24.88 | C |
| ATOM | 6619 | CG2 | VAL | M | 205 | 64.150 | 1.645 | −17.686 | 1.00 | 24.90 | C |
| ATOM | 6620 | C | VAL | M | 205 | 65.996 | −0.312 | −20.454 | 1.00 | 23.53 | C |
| ATOM | 6621 | O | VAL | M | 205 | 65.468 | −1.357 | −20.873 | 1.00 | 23.36 | O |
| ATOM | 6622 | N | THR | M | 206 | 67.308 | −0.103 | −20.486 | 1.00 | 23.20 | N |
| ATOM | 6623 | CA | THR | M | 206 | 68.219 | −1.106 | −21.007 | 1.00 | 23.22 | C |
| ATOM | 6624 | CB | THR | M | 206 | 68.897 | −0.607 | −22.298 | 1.00 | 23.15 | C |
| ATOM | 6625 | OG1 | THR | M | 206 | 67.908 | −0.486 | −23.330 | 1.00 | 24.38 | O |
| ATOM | 6626 | CG2 | THR | M | 206 | 69.968 | −1.573 | −22.754 | 1.00 | 22.98 | C |
| ATOM | 6627 | C | THR | M | 206 | 69.257 | −1.492 | −19.962 | 1.00 | 23.13 | C |
| ATOM | 6628 | O | THR | M | 206 | 69.926 | −0.631 | −19.394 | 1.00 | 23.13 | O |
| ATOM | 6629 | N | LYS | M | 207 | 69.388 | −2.785 | −19.705 | 1.00 | 22.58 | N |
| ATOM | 6630 | CA | LYS | M | 207 | 70.468 | −3.268 | −18.852 | 1.00 | 22.71 | C |
| ATOM | 6631 | CB | LYS | M | 207 | 69.935 | −4.097 | −17.678 | 1.00 | 22.67 | C |
| ATOM | 6632 | CG | LYS | M | 207 | 68.999 | −3.364 | −16.719 | 1.00 | 22.72 | C |
| ATOM | 6633 | CD | LYS | M | 207 | 69.615 | −2.114 | −16.132 | 1.00 | 25.31 | C |
| ATOM | 6634 | CE | LYS | M | 207 | 68.824 | −1.626 | −14.931 | 1.00 | 26.51 | C |
| ATOM | 6635 | NZ | LYS | M | 207 | 69.105 | −0.191 | −14.668 | 1.00 | 27.71 | N |
| ATOM | 6636 | C | LYS | M | 207 | 71.390 | −4.108 | −19.708 | 1.00 | 22.70 | C |
| ATOM | 6637 | O | LYS | M | 207 | 70.925 | −4.910 | −20.518 | 1.00 | 22.10 | O |
| ATOM | 6638 | N | SER | M | 208 | 72.698 | −3.914 | −19.555 | 1.00 | 22.83 | N |
| ATOM | 6639 | CA | SER | M | 208 | 73.631 | −4.626 | −20.409 | 1.00 | 23.50 | C |
| ATOM | 6640 | CB | SER | M | 208 | 73.953 | −3.801 | −21.653 | 1.00 | 23.20 | C |
| ATOM | 6641 | OG | SER | M | 208 | 74.685 | −2.650 | −21.315 | 1.00 | 24.68 | O |
| ATOM | 6642 | C | SER | M | 208 | 74.904 | −5.035 | −19.683 | 1.00 | 24.03 | C |
| ATOM | 6643 | O | SER | M | 208 | 75.178 | −4.566 | −18.576 | 1.00 | 24.39 | O |
| ATOM | 6644 | N | PHE | M | 209 | 75.647 | −5.952 | −20.292 | 1.00 | 24.31 | N |
| ATOM | 6645 | CA | PHE | M | 209 | 76.990 | −6.273 | −19.840 | 1.00 | 24.92 | C |
| ATOM | 6646 | CB | PHE | M | 209 | 77.010 | −7.473 | −18.873 | 1.00 | 24.66 | C |
| ATOM | 6647 | CG | PHE | M | 209 | 76.579 | −8.787 | −19.492 | 1.00 | 24.98 | C |
| ATOM | 6648 | CD1 | PHE | M | 209 | 77.492 | −9.599 | −20.160 | 1.00 | 24.39 | C |
| ATOM | 6649 | CE1 | PHE | M | 209 | 77.092 | −10.821 | −20.718 | 1.00 | 24.96 | C |
| ATOM | 6650 | CZ | PHE | M | 209 | 75.773 | −11.245 | −20.595 | 1.00 | 23.61 | C |
| ATOM | 6651 | CE2 | PHE | M | 209 | 74.860 | −10.453 | −19.918 | 1.00 | 24.38 | C |
| ATOM | 6652 | CD2 | PHE | M | 209 | 75.264 | −9.233 | −19.363 | 1.00 | 24.95 | C |
| ATOM | 6653 | C | PHE | M | 209 | 77.880 | −6.520 | −21.048 | 1.00 | 25.31 | C |
| ATOM | 6654 | O | PHE | M | 209 | 77.400 | −6.846 | −22.133 | 1.00 | 25.31 | O |
| ATOM | 6655 | N | ASN | M | 210 | 79.178 | −6.338 | −20.848 | 1.00 | 25.95 | N |
| ATOM | 6656 | CA | ASN | M | 210 | 80.167 | −6.706 | −21.841 | 1.00 | 26.69 | C |
| ATOM | 6657 | CB | ASN | M | 210 | 81.251 | −5.630 | −21.925 | 1.00 | 26.72 | C |
| ATOM | 6658 | CG | ASN | M | 210 | 80.693 | −4.264 | −22.323 | 1.00 | 27.02 | C |
| ATOM | 6659 | OD1 | ASN | M | 210 | 79.734 | −4.163 | −23.095 | 1.00 | 26.41 | O |
| ATOM | 6660 | ND2 | ASN | M | 210 | 81.300 | −3.206 | −21.797 | 1.00 | 27.34 | N |
| ATOM | 6661 | C | ASN | M | 210 | 80.747 | −8.062 | −21.455 | 1.00 | 27.24 | C |
| ATOM | 6662 | O | ASN | M | 210 | 81.264 | −8.231 | −20.343 | 1.00 | 27.31 | O |
| ATOM | 6663 | N | ARG | M | 211 | 80.617 | −9.033 | −22.357 | 1.00 | 28.04 | N |
| ATOM | 6664 | CA | ARG | M | 211 | 81.121 | −10.389 | −22.126 | 1.00 | 28.96 | C |
| ATOM | 6665 | CB | ARG | M | 211 | 81.029 | −11.221 | −23.411 | 1.00 | 28.54 | C |
| ATOM | 6666 | CG | ARG | M | 211 | 81.576 | −12.642 | −23.288 | 1.00 | 27.68 | C |
| ATOM | 6667 | CD | ARG | M | 211 | 81.537 | −13.411 | −24.612 | 1.00 | 25.87 | C |
| ATOM | 6668 | NE | ARG | M | 211 | 82.085 | −12.635 | −25.721 | 1.00 | 23.82 | N |
| ATOM | 6669 | CZ | ARG | M | 211 | 83.380 | −12.514 | −25.994 | 1.00 | 23.43 | C |
| ATOM | 6670 | NH1 | ARG | M | 211 | 84.287 | −13.128 | −25.246 | 1.00 | 22.36 | N |
| ATOM | 6671 | NH2 | ARG | M | 211 | 83.766 | −11.771 | −27.023 | 1.00 | 21.17 | N |
| ATOM | 6672 | C | ARG | M | 211 | 82.564 | −10.323 | −21.624 | 1.00 | 30.27 | C |
| ATOM | 6673 | O | ARG | M | 211 | 83.415 | −9.674 | −22.244 | 1.00 | 30.13 | O |
| ATOM | 6674 | N | GLY | M | 212 | 82.820 | −10.969 | −20.488 | 1.00 | 31.64 | N |
| ATOM | 6675 | CA | GLY | M | 212 | 84.149 | −10.954 | −19.885 | 1.00 | 33.39 | C |
| ATOM | 6676 | C | GLY | M | 212 | 84.288 | −9.973 | −18.732 | 1.00 | 34.50 | C |
| ATOM | 6677 | O | GLY | M | 212 | 84.839 | −10.323 | −17.685 | 1.00 | 34.82 | O |
| ATOM | 6678 | N | GLU | M | 213 | 83.798 | −8.747 | −18.923 | 1.00 | 35.22 | N |
| ATOM | 6679 | CA | GLU | M | 213 | 83.893 | −7.700 | −17.897 | 1.00 | 35.89 | C |
| ATOM | 6680 | CB | GLU | M | 213 | 83.698 | −6.315 | −18.517 | 1.00 | 36.06 | C |
| ATOM | 6681 | CG | GLU | M | 213 | 84.793 | −5.929 | −19.500 | 1.00 | 36.84 | C |
| ATOM | 6682 | CD | GLU | M | 213 | 84.389 | −4.792 | −20.424 | 1.00 | 38.13 | C |
| ATOM | 6683 | OE1 | GLU | M | 213 | 83.560 | −3.948 | −20.016 | 1.00 | 39.42 | O |
| ATOM | 6684 | OE2 | GLU | M | 213 | 84.907 | −4.741 | −21.561 | 1.00 | 38.02 | O |
| ATOM | 6685 | C | GLU | M | 213 | 82.892 | −7.915 | −16.766 | 1.00 | 36.07 | C |
| ATOM | 6686 | O | GLU | M | 213 | 83.149 | −8.689 | −15.840 | 1.00 | 36.34 | O |
| ATOM | 6687 | O | HOH | S | 1 | 66.884 | −28.058 | 22.342 | 1.00 | 18.70 | O |
| ATOM | 6688 | O | HOH | S | 2 | 27.899 | −35.072 | −13.062 | 1.00 | 21.04 | O |
| ATOM | 6689 | O | HOH | S | 3 | 30.634 | −13.987 | 14.268 | 1.00 | 16.87 | O |
| ATOM | 6690 | O | HOH | S | 4 | 60.421 | −23.403 | 18.687 | 1.00 | 18.75 | O |
| ATOM | 6691 | O | HOH | S | 5 | 53.587 | −40.006 | 11.549 | 1.00 | 19.02 | O |
| ATOM | 6692 | O | HOH | S | 6 | 21.268 | −15.198 | −8.212 | 1.00 | 19.27 | O |
| ATOM | 6693 | O | HOH | S | 7 | 53.507 | −44.593 | 24.947 | 1.00 | 21.35 | O |
| ATOM | 6694 | O | HOH | S | 8 | 63.117 | −44.543 | 13.709 | 1.00 | 15.71 | O |
| ATOM | 6695 | O | HOH | S | 9 | 28.384 | −41.003 | −26.914 | 1.00 | 22.62 | O |
| ATOM | 6696 | O | HOH | S | 10 | 29.018 | −28.186 | −18.612 | 1.00 | 17.88 | O |
| ATOM | 6697 | O | HOH | S | 11 | 47.921 | −36.942 | 24.164 | 1.00 | 16.12 | O |
| ATOM | 6698 | O | HOH | S | 12 | 29.552 | −28.877 | −9.754 | 1.00 | 20.49 | O |
| ATOM | 6699 | O | HOH | S | 13 | 54.215 | −12.139 | −8.745 | 1.00 | 14.88 | O |
| ATOM | 6700 | O | HOH | S | 14 | 22.663 | −21.591 | −1.124 | 1.00 | 13.63 | O |
| ATOM | 6701 | O | HOH | S | 15 | 52.854 | −1.932 | −20.068 | 1.00 | 15.32 | O |
| ATOM | 6702 | O | HOH | S | 16 | 59.307 | −14.375 | 13.086 | 1.00 | 19.34 | O |
| ATOM | 6703 | O | HOH | S | 17 | 55.534 | −38.323 | 26.467 | 1.00 | 19.72 | O |
| ATOM | 6704 | O | HOH | S | 18 | 28.770 | −31.349 | −3.431 | 1.00 | 20.45 | O |
| ATOM | 6705 | O | HOH | S | 19 | 28.407 | −35.778 | −26.748 | 1.00 | 26.47 | O |
| ATOM | 6706 | O | HOH | S | 20 | 38.621 | −45.455 | 4.538 | 1.00 | 15.98 | O |
| ATOM | 6707 | O | HOH | S | 21 | 68.963 | −30.561 | 19.534 | 1.00 | 23.77 | O |
| ATOM | 6708 | O | HOH | S | 22 | 15.711 | −23.371 | −14.803 | 1.00 | 24.27 | O |
| ATOM | 6709 | O | HOH | S | 23 | 38.163 | −47.047 | −18.934 | 1.00 | 19.31 | O |
| ATOM | 6710 | O | HOH | S | 24 | 53.097 | −34.713 | 19.089 | 1.00 | 16.98 | O |
| ATOM | 6711 | O | HOH | S | 25 | 25.506 | −9.817 | −7.038 | 1.00 | 20.87 | O |
| ATOM | 6712 | O | HOH | S | 26 | 22.016 | −20.373 | 23.449 | 1.00 | 25.20 | O |
| ATOM | 6713 | O | HOH | S | 27 | 58.950 | −32.681 | −23.667 | 1.00 | 15.57 | O |
| ATOM | 6714 | O | HOH | S | 28 | 66.786 | −23.589 | 11.823 | 1.00 | 36.74 | O |
| ATOM | 6715 | O | HOH | S | 29 | 33.503 | −26.710 | −23.762 | 1.00 | 26.27 | O |
| ATOM | 6716 | O | HOH | S | 30 | 49.153 | −34.531 | 24.711 | 1.00 | 15.23 | O |
| ATOM | 6717 | O | HOH | S | 31 | 28.478 | −43.889 | −25.603 | 1.00 | 25.16 | O |
| ATOM | 6718 | O | HOH | S | 32 | 22.988 | −48.076 | −20.150 | 1.00 | 17.46 | O |
| ATOM | 6719 | O | HOH | S | 33 | 70.271 | −24.272 | 2.530 | 1.00 | 10.67 | O |
| ATOM | 6720 | O | HOH | S | 34 | 55.865 | −42.383 | 7.301 | 1.00 | 20.41 | O |
| ATOM | 6721 | O | HOH | S | 35 | 24.866 | −9.174 | 33.199 | 1.00 | 17.60 | O |
| ATOM | 6722 | O | HOH | S | 36 | 48.274 | −45.267 | −16.911 | 1.00 | 24.67 | O |
| ATOM | 6723 | O | HOH | S | 37 | 45.183 | −38.956 | −28.274 | 1.00 | 22.63 | O |
| ATOM | 6724 | O | HOH | S | 38 | 52.859 | −51.604 | 21.498 | 1.00 | 29.12 | O |
| ATOM | 6725 | O | HOH | S | 39 | 61.321 | −43.010 | 6.743 | 1.00 | 24.70 | O |
| ATOM | 6726 | O | HOH | S | 40 | 67.110 | −30.672 | 16.096 | 1.00 | 22.84 | O |
| ATOM | 6727 | O | HOH | S | 41 | 46.305 | 1.133 | −13.045 | 1.00 | 19.06 | O |
| ATOM | 6728 | O | HOH | S | 42 | 46.021 | −3.371 | 11.914 | 1.00 | 17.81 | O |
| ATOM | 6729 | O | HOH | S | 43 | 60.119 | −21.069 | −4.860 | 1.00 | 22.64 | O |
| ATOM | 6730 | O | HOH | S | 44 | 32.197 | −30.652 | −24.825 | 1.00 | 19.78 | O |
| ATOM | 6731 | O | HOH | S | 45 | 73.535 | −19.693 | −31.000 | 1.00 | 19.19 | O |
| ATOM | 6732 | O | HOH | S | 46 | 53.010 | −33.612 | 3.754 | 1.00 | 36.15 | O |
| ATOM | 6733 | O | HOH | S | 47 | 61.958 | −41.193 | 25.472 | 1.00 | 22.43 | O |
| ATOM | 6734 | O | HOH | S | 48 | 47.049 | −37.252 | −9.129 | 1.00 | 27.68 | O |
| ATOM | 6735 | O | HOH | S | 49 | 32.401 | −44.962 | 5.518 | 1.00 | 20.74 | O |
| ATOM | 6736 | O | HOH | S | 50 | 57.072 | −13.689 | 4.954 | 1.00 | 24.50 | O |
| ATOM | 6737 | O | HOH | S | 51 | 27.544 | −13.320 | 3.210 | 1.00 | 11.93 | O |
| ATOM | 6738 | O | HOH | S | 52 | 53.255 | −17.469 | −9.892 | 1.00 | 19.20 | O |
| ATOM | 6739 | O | HOH | S | 53 | 46.095 | −24.024 | 9.496 | 1.00 | 31.74 | O |
| ATOM | 6740 | O | HOH | S | 54 | 60.877 | −17.645 | 18.038 | 1.00 | 29.44 | O |
| ATOM | 6741 | O | HOH | S | 55 | 18.464 | −13.366 | −11.206 | 1.00 | 38.88 | O |
| ATOM | 6742 | O | HOH | S | 56 | 38.038 | −11.303 | 22.625 | 1.00 | 22.67 | O |
| ATOM | 6743 | O | HOH | S | 57 | 24.064 | −19.326 | −0.456 | 1.00 | 17.79 | O |
| ATOM | 6744 | O | HOH | S | 58 | 44.475 | −2.598 | −5.507 | 1.00 | 21.33 | O |
| ATOM | 6745 | O | HOH | S | 59 | 75.918 | −16.682 | −1.172 | 1.00 | 13.05 | O |
| ATOM | 6746 | O | HOH | S | 60 | 23.697 | −37.210 | 25.995 | 1.00 | 17.31 | O |
| ATOM | 6747 | O | HOH | S | 61 | 34.988 | −44.188 | −26.967 | 1.00 | 29.24 | O |
| ATOM | 6748 | O | HOH | S | 62 | 28.245 | −47.017 | −22.763 | 1.00 | 23.68 | O |
| ATOM | 6749 | O | HOH | S | 63 | 31.168 | −23.758 | −3.808 | 1.00 | 20.86 | O |
| ATOM | 6750 | O | HOH | S | 64 | 31.003 | −20.606 | 2.608 | 1.00 | 39.35 | O |
| ATOM | 6751 | O | HOH | S | 65 | 34.068 | −38.755 | −33.324 | 1.00 | 27.39 | O |
| ATOM | 6752 | O | HOH | S | 66 | 33.709 | −44.962 | 10.139 | 1.00 | 25.22 | O |
| ATOM | 6753 | O | HOH | S | 67 | 62.116 | −20.054 | 23.759 | 1.00 | 25.13 | O |
| ATOM | 6754 | O | HOH | S | 68 | 58.291 | −22.292 | 1.747 | 1.00 | 24.19 | O |
| ATOM | 6755 | O | HOH | S | 69 | 25.541 | −45.438 | −23.421 | 1.00 | 26.96 | O |
| ATOM | 6756 | O | HOH | S | 70 | 54.761 | −28.446 | 30.903 | 1.00 | 24.25 | O |
| ATOM | 6757 | O | HOH | S | 71 | 16.648 | −21.232 | −13.397 | 1.00 | 16.67 | O |
| ATOM | 6758 | O | HOH | S | 72 | 37.755 | −31.169 | −4.065 | 1.00 | 29.50 | O |
| ATOM | 6759 | O | HOH | S | 73 | 59.157 | −29.195 | 4.131 | 1.00 | 34.00 | O |
| ATOM | 6760 | O | HOH | S | 74 | 62.378 | −20.846 | 0.012 | 1.00 | 19.84 | O |
| ATOM | 6761 | O | HOH | S | 75 | 65.855 | −40.056 | 23.498 | 1.00 | 17.16 | O |
| ATOM | 6762 | O | HOH | S | 76 | 16.374 | −17.190 | −12.731 | 1.00 | 26.26 | O |
| ATOM | 6763 | O | HOH | S | 77 | 47.068 | 0.206 | −17.849 | 1.00 | 19.59 | O |
| ATOM | 6764 | O | HOH | S | 78 | 36.079 | −27.894 | −6.389 | 1.00 | 26.22 | O |
| ATOM | 6765 | O | HOH | S | 79 | 56.804 | 2.805 | −10.842 | 1.00 | 17.94 | O |
| ATOM | 6766 | O | HOH | S | 80 | 47.809 | −46.683 | −27.536 | 1.00 | 20.94 | O |
| ATOM | 6767 | O | HOH | S | 81 | 46.035 | −30.565 | 7.916 | 1.00 | 29.48 | O |
| ATOM | 6768 | O | HOH | S | 82 | 39.626 | −9.164 | −21.915 | 1.00 | 32.39 | O |
| ATOM | 6769 | O | HOH | S | 83 | 34.499 | −39.146 | 7.980 | 1.00 | 19.96 | O |
| ATOM | 6770 | O | HOH | S | 84 | 53.475 | −42.887 | −15.935 | 1.00 | 27.14 | O |
| ATOM | 6771 | O | HOH | S | 85 | 61.174 | −42.300 | 20.508 | 1.00 | 29.27 | O |
| ATOM | 6772 | O | HOH | S | 86 | 63.519 | −27.682 | −3.349 | 1.00 | 19.54 | O |
| ATOM | 6773 | O | HOH | S | 87 | 8.412 | −13.618 | 6.506 | 1.00 | 18.31 | O |
| ATOM | 6774 | O | HOH | S | 88 | 33.332 | −7.244 | 28.084 | 1.00 | 18.11 | O |
| ATOM | 6775 | O | HOH | S | 89 | 44.027 | −24.838 | −10.737 | 1.00 | 30.75 | O |
| ATOM | 6776 | O | HOH | S | 90 | 56.274 | −24.830 | −14.257 | 1.00 | 29.35 | O |
| ATOM | 6777 | O | HOH | S | 91 | 72.310 | −16.618 | −16.995 | 1.00 | 19.80 | O |
| ATOM | 6778 | O | HOH | S | 92 | 23.479 | −11.114 | −12.513 | 1.00 | 25.59 | O |
| ATOM | 6779 | O | HOH | S | 93 | 42.257 | −21.732 | −14.379 | 1.00 | 29.24 | O |
| ATOM | 6780 | O | HOH | S | 94 | 74.607 | −20.086 | −16.503 | 1.00 | 18.71 | O |
| ATOM | 6781 | O | HOH | S | 95 | 51.943 | −15.182 | 3.080 | 1.00 | 26.43 | O |
| ATOM | 6782 | O | HOH | S | 96 | 29.797 | −15.257 | 27.760 | 1.00 | 31.82 | O |
| ATOM | 6783 | O | HOH | S | 97 | 65.978 | −32.122 | 9.025 | 1.00 | 37.00 | O |
| ATOM | 6784 | O | HOH | S | 98 | 57.896 | −43.219 | 5.847 | 1.00 | 29.73 | O |
| ATOM | 6785 | O | HOH | S | 99 | 43.575 | −11.674 | −4.003 | 1.00 | 28.80 | O |
| ATOM | 6786 | O | HOH | S | 100 | 40.838 | −8.069 | 23.301 | 1.00 | 20.61 | O |
| ATOM | 6787 | O | HOH | S | 101 | 59.760 | −39.530 | −16.851 | 1.00 | 21.26 | O |
| ATOM | 6788 | O | HOH | S | 102 | 22.420 | −32.687 | 8.334 | 1.00 | 29.82 | O |
| ATOM | 6789 | O | HOH | S | 103 | 60.044 | −11.807 | 6.822 | 1.00 | 28.66 | O |
| ATOM | 6790 | O | HOH | S | 104 | 50.243 | −1.268 | −20.296 | 1.00 | 20.48 | O |
| ATOM | 6791 | O | HOH | S | 105 | 56.427 | −33.227 | 3.252 | 1.00 | 27.25 | O |
| ATOM | 6792 | O | HOH | S | 106 | 29.527 | −13.009 | −23.796 | 1.00 | 27.20 | O |
| ATOM | 6793 | O | HOH | S | 107 | 50.492 | −31.844 | 27.033 | 1.00 | 24.00 | O |
| ATOM | 6794 | O | HOH | S | 108 | 16.392 | −17.883 | −17.055 | 1.00 | 26.30 | O |
| ATOM | 6795 | O | HOH | S | 109 | 47.079 | −8.812 | 22.829 | 1.00 | 29.90 | O |
| ATOM | 6796 | O | HOH | S | 110 | 67.083 | −28.306 | 13.095 | 1.00 | 26.14 | O |
| ATOM | 6797 | O | HOH | S | 111 | 59.252 | −24.465 | −6.145 | 1.00 | 22.69 | O |
| ATOM | 6798 | O | HOH | S | 112 | 25.285 | −8.838 | −0.480 | 1.00 | 21.34 | O |
| ATOM | 6799 | O | HOH | S | 113 | 19.266 | −21.400 | 20.899 | 1.00 | 29.27 | O |
| ATOM | 6800 | O | HOH | S | 114 | 48.258 | −54.751 | 11.856 | 1.00 | 22.00 | O |
| ATOM | 6801 | O | HOH | S | 115 | 54.278 | −45.764 | −28.540 | 1.00 | 24.00 | O |
| ATOM | 6802 | O | HOH | S | 116 | 36.830 | −17.238 | 16.475 | 1.00 | 30.75 | O |
| ATOM | 6803 | O | HOH | S | 117 | 58.308 | −11.150 | 16.601 | 1.00 | 27.71 | O |
| ATOM | 6804 | O | HOH | S | 118 | 43.329 | −14.699 | −11.308 | 1.00 | 29.85 | O |
| ATOM | 6805 | O | HOH | S | 119 | 37.279 | −52.305 | 26.635 | 1.00 | 25.18 | O |
| ATOM | 6806 | O | HOH | S | 120 | 65.834 | −19.936 | 16.075 | 1.00 | 34.05 | O |
| ATOM | 6807 | O | HOH | S | 121 | 43.038 | −47.919 | 29.037 | 1.00 | 39.50 | O |
| ATOM | 6808 | O | HOH | S | 122 | 64.642 | −32.472 | 31.453 | 1.00 | 23.63 | O |
| ATOM | 6809 | O | HOH | S | 123 | 42.847 | −21.459 | −17.099 | 1.00 | 33.48 | O |
| ATOM | 6810 | O | HOH | S | 124 | 52.644 | −24.711 | 4.150 | 1.00 | 26.91 | O |
| ATOM | 6811 | O | HOH | S | 125 | 63.768 | −42.785 | 20.219 | 1.00 | 37.56 | O |
| ATOM | 6812 | O | HOH | S | 126 | 51.258 | −37.950 | −8.593 | 1.00 | 25.79 | O |
| ATOM | 6813 | O | HOH | S | 127 | 20.025 | −21.987 | −3.747 | 1.00 | 23.68 | O |
| ATOM | 6814 | O | HOH | S | 128 | 72.157 | −27.577 | −19.028 | 1.00 | 23.16 | O |
| ATOM | 6815 | O | HOH | S | 129 | 46.895 | −9.118 | −5.373 | 1.00 | 23.67 | O |
| ATOM | 6816 | O | HOH | S | 130 | 29.296 | −36.296 | 24.681 | 1.00 | 27.38 | O |
| ATOM | 6817 | O | HOH | S | 131 | 19.218 | −0.377 | 14.003 | 1.00 | 34.53 | O |
| ATOM | 6818 | O | HOH | S | 132 | 42.689 | −19.928 | −11.284 | 1.00 | 28.50 | O |
| ATOM | 6819 | O | HOH | S | 133 | 65.996 | −10.936 | 2.737 | 1.00 | 24.85 | O |
| ATOM | 6820 | O | HOH | S | 134 | 24.895 | −7.270 | −8.034 | 1.00 | 34.34 | O |
| ATOM | 6821 | O | HOH | S | 135 | 15.826 | −22.001 | −10.952 | 1.00 | 25.27 | O |
| ATOM | 6822 | O | HOH | S | 136 | 65.619 | −1.249 | −13.553 | 1.00 | 30.66 | O |
| ATOM | 6823 | O | HOH | S | 137 | 59.408 | 5.094 | −14.319 | 1.00 | 19.67 | O |
| ATOM | 6824 | O | HOH | S | 138 | 62.518 | −17.498 | −19.136 | 1.00 | 33.65 | O |
| ATOM | 6825 | O | HOH | S | 139 | 39.595 | −3.525 | 21.793 | 1.00 | 23.47 | O |
| ATOM | 6826 | O | HOH | S | 140 | 46.544 | −33.483 | 4.734 | 1.00 | 29.41 | O |
| ATOM | 6827 | O | HOH | S | 141 | 10.974 | −25.782 | 34.245 | 1.00 | 22.31 | O |
| ATOM | 6828 | O | HOH | S | 142 | 63.205 | −51.205 | 9.123 | 1.00 | 26.08 | O |
| ATOM | 6829 | O | HOH | S | 143 | 46.115 | −41.281 | −5.271 | 1.00 | 23.88 | O |
| ATOM | 6830 | O | HOH | S | 144 | 49.203 | −30.054 | 28.470 | 1.00 | 33.45 | O |
| ATOM | 6831 | O | HOH | S | 145 | 67.627 | −27.924 | 2.004 | 1.00 | 35.13 | O |
| ATOM | 6832 | O | HOH | S | 146 | 47.551 | −36.880 | −6.335 | 1.00 | 28.54 | O |
| ATOM | 6833 | O | HOH | S | 147 | 68.369 | −11.716 | 3.111 | 1.00 | 30.93 | O |
| ATOM | 6834 | O | HOH | S | 148 | 20.071 | −2.877 | 33.157 | 1.00 | 26.79 | O |
| ATOM | 6835 | O | HOH | S | 149 | 47.708 | −30.796 | 6.094 | 1.00 | 37.10 | O |
| ATOM | 6836 | O | HOH | S | 150 | 25.327 | −38.436 | −9.538 | 1.00 | 42.47 | O |
| ATOM | 6837 | O | HOH | S | 151 | 45.547 | −7.451 | −13.639 | 1.00 | 32.68 | O |
| ATOM | 6838 | O | HOH | S | 152 | 58.348 | −17.562 | −24.423 | 1.00 | 22.73 | O |
| ATOM | 6839 | O | HOH | S | 153 | 44.423 | −27.783 | −13.978 | 1.00 | 32.62 | O |
| ATOM | 6840 | O | HOH | S | 154 | 40.795 | −23.940 | 15.381 | 1.00 | 30.47 | O |
| ATOM | 6841 | O | HOH | S | 155 | 54.290 | −17.587 | −18.077 | 1.00 | 27.43 | O |
| ATOM | 6842 | O | HOH | S | 156 | 32.264 | −50.070 | −17.150 | 1.00 | 20.94 | O |
| ATOM | 6843 | O | HOH | S | 157 | 13.612 | −8.760 | 17.169 | 1.00 | 19.90 | O |
| ATOM | 6844 | O | HOH | S | 158 | 51.681 | −44.010 | 27.326 | 1.00 | 23.82 | O |
| ATOM | 6845 | O | HOH | S | 159 | 70.158 | −3.928 | −12.588 | 1.00 | 20.42 | O |
| ATOM | 6846 | O | HOH | S | 160 | 57.162 | −39.956 | 27.998 | 1.00 | 24.18 | O |
| ATOM | 6847 | O | HOH | S | 161 | 19.810 | −10.578 | −9.734 | 1.00 | 35.60 | O |
| ATOM | 6848 | O | HOH | S | 162 | 15.838 | −18.717 | −14.678 | 1.00 | 23.11 | O |
| ATOM | 6849 | O | HOH | S | 163 | 49.996 | −34.656 | 27.580 | 1.00 | 27.96 | O |
| ATOM | 6850 | O | HOH | S | 164 | 41.617 | −29.507 | 6.888 | 1.00 | 34.99 | O |
| ATOM | 6851 | O | HOH | S | 165 | 14.523 | −24.651 | −11.984 | 1.00 | 33.29 | O |
| ATOM | 6852 | O | HOH | S | 166 | 48.507 | −43.834 | −8.389 | 1.00 | 26.38 | O |
| ATOM | 6853 | O | HOH | S | 167 | 63.552 | −23.689 | −26.981 | 1.00 | 31.69 | O |
| ATOM | 6854 | O | HOH | S | 168 | 16.019 | −5.012 | 20.892 | 1.00 | 24.78 | O |
| ATOM | 6855 | O | HOH | S | 169 | 21.279 | −31.114 | −4.495 | 1.00 | 23.83 | O |
| ATOM | 6856 | O | HOH | S | 170 | 15.931 | −30.414 | −24.227 | 1.00 | 23.31 | O |
| ATOM | 6857 | O | HOH | S | 171 | 57.385 | −34.971 | −27.773 | 1.00 | 20.70 | O |
| ATOM | 6858 | O | HOH | S | 172 | 58.076 | −27.367 | −11.652 | 1.00 | 41.14 | O |
| ATOM | 6859 | O | HOH | S | 173 | 14.790 | −0.092 | 8.122 | 1.00 | 21.81 | O |
| ATOM | 6860 | O | HOH | S | 174 | 69.841 | −3.000 | 4.606 | 1.00 | 29.06 | O |
| ATOM | 6861 | O | HOH | S | 175 | 16.518 | −27.300 | −8.377 | 1.00 | 35.00 | O |
| ATOM | 6862 | O | HOH | S | 176 | 67.763 | −16.816 | 4.793 | 1.00 | 23.12 | O |
| ATOM | 6863 | O | HOH | S | 177 | 35.533 | −30.463 | −27.998 | 1.00 | 35.42 | O |
| ATOM | 6864 | O | HOH | S | 178 | 38.279 | 0.438 | 21.458 | 1.00 | 22.29 | O |
| ATOM | 6865 | O | HOH | S | 179 | 51.357 | −45.900 | −16.368 | 1.00 | 31.63 | O |
| ATOM | 6866 | O | HOH | S | 180 | 45.388 | −19.218 | 5.378 | 1.00 | 44.70 | O |
| ATOM | 6867 | O | HOH | S | 181 | 30.070 | −14.377 | 11.820 | 1.00 | 29.80 | O |
| ATOM | 6868 | O | HOH | S | 182 | 27.098 | −12.806 | 9.485 | 1.00 | 29.51 | O |
| ATOM | 6869 | O | HOH | S | 183 | 43.693 | −7.016 | 24.000 | 1.00 | 31.74 | O |
| ATOM | 6870 | O | HOH | S | 184 | 20.923 | −9.232 | 6.428 | 1.00 | 31.20 | O |
| ATOM | 6871 | O | HOH | S | 185 | 23.942 | −1.779 | 22.561 | 1.00 | 28.59 | O |
| ATOM | 6872 | O | HOH | S | 186 | 57.209 | −45.946 | 24.716 | 1.00 | 45.50 | O |
| ATOM | 6873 | O | HOH | S | 187 | 12.566 | −32.539 | −7.503 | 1.00 | 32.74 | O |
| ATOM | 6874 | O | HOH | S | 188 | 71.589 | −21.997 | 3.801 | 1.00 | 35.25 | O |
| ATOM | 6875 | O | HOH | S | 189 | 48.356 | −54.465 | 15.313 | 1.00 | 29.62 | O |
| ATOM | 6876 | O | HOH | S | 190 | 73.816 | −28.836 | −11.543 | 1.00 | 26.20 | O |
| ATOM | 6877 | O | HOH | S | 191 | 5.663 | −7.946 | 26.570 | 1.00 | 43.96 | O |
| ATOM | 6878 | O | HOH | S | 192 | 68.816 | −4.640 | −35.082 | 1.00 | 22.29 | O |
| ATOM | 6879 | O | HOH | S | 193 | 15.041 | −19.857 | −25.390 | 1.00 | 32.80 | O |
| ATOM | 6880 | O | HOH | S | 194 | 29.953 | −51.756 | −19.933 | 1.00 | 35.91 | O |
| ATOM | 6881 | O | HOH | S | 195 | 37.891 | −22.276 | 9.430 | 1.00 | 37.20 | O |
| ATOM | 6882 | O | HOH | S | 196 | 38.718 | −32.127 | 8.096 | 1.00 | 39.96 | O |
| ATOM | 6883 | O | HOH | S | 197 | 29.294 | −9.750 | 12.109 | 1.00 | 27.14 | O |
| ATOM | 6884 | O | HOH | S | 198 | 25.030 | −30.963 | −3.056 | 1.00 | 17.10 | O |
| ATOM | 6885 | O | HOH | S | 199 | 68.041 | −26.801 | 10.804 | 1.00 | 49.34 | O |
| ATOM | 6886 | O | HOH | S | 200 | 44.893 | −46.230 | −10.867 | 1.00 | 35.80 | O |
| ATOM | 6887 | O | HOH | S | 201 | 47.269 | −47.475 | −15.086 | 1.00 | 43.42 | O |
| ATOM | 6888 | O | HOH | S | 202 | 38.864 | −34.873 | −1.015 | 1.00 | 47.69 | O |
| ATOM | 6889 | O | HOH | S | 203 | 71.746 | −34.302 | 25.098 | 1.00 | 41.70 | O |
| ATOM | 6890 | O | HOH | S | 204 | 37.064 | −26.023 | 15.321 | 1.00 | 49.19 | O |
| ATOM | 6891 | O | HOH | S | 205 | 53.722 | −27.955 | 2.261 | 1.00 | 29.03 | O |
| ATOM | 6892 | O | HOH | S | 206 | 58.407 | −17.389 | 25.050 | 1.00 | 35.63 | O |
| ATOM | 6893 | O | HOH | S | 207 | 26.236 | −22.413 | 28.371 | 1.00 | 24.45 | O |
| ATOM | 6894 | O | HOH | S | 208 | 30.199 | −49.497 | 13.490 | 1.00 | 39.76 | O |
| ATOM | 6895 | O | HOH | S | 209 | 29.090 | −48.756 | 11.083 | 1.00 | 38.55 | O |
| ATOM | 6896 | O | HOH | S | 210 | 37.290 | −56.198 | 14.270 | 1.00 | 39.47 | O |
| ATOM | 6897 | O | HOH | S | 211 | 10.641 | −31.907 | 18.941 | 1.00 | 48.42 | O |
| ATOM | 6898 | O | HOH | S | 212 | 23.107 | −23.177 | 20.243 | 1.00 | 22.81 | O |
| ATOM | 6899 | O | HOH | S | 213 | 60.205 | −27.938 | 32.529 | 1.00 | 39.89 | O |
| ATOM | 6900 | O | HOH | S | 214 | 54.333 | −48.003 | −21.857 | 1.00 | 40.45 | O |
| ATOM | 6901 | O | HOH | S | 215 | 38.774 | −51.644 | −9.358 | 1.00 | 32.03 | O |
| ATOM | 6902 | O | HOH | S | 216 | 70.230 | −31.393 | −17.014 | 1.00 | 44.60 | O |
| ATOM | 6903 | O | HOH | S | 217 | 56.282 | −20.812 | −18.503 | 1.00 | 85.10 | O |
| ATOM | 6904 | O | HOH | S | 218 | 57.856 | −25.146 | −26.016 | 1.00 | 40.10 | O |
| ATOM | 6905 | O | HOH | S | 219 | 78.226 | −8.065 | 4.099 | 1.00 | 37.29 | O |
| ATOM | 6906 | O | HOH | S | 220 | 36.413 | 0.400 | −3.677 | 1.00 | 35.86 | O |
| ATOM | 6907 | O | HOH | S | 221 | 17.506 | −25.796 | −26.186 | 1.00 | 44.64 | O |
| ATOM | 6908 | O | HOH | S | 222 | 52.604 | −52.682 | 13.666 | 1.00 | 31.96 | O |
| ATOM | 6909 | O | HOH | S | 223 | 31.606 | −40.676 | 29.663 | 1.00 | 23.23 | O |
| ATOM | 6910 | O | HOH | S | 224 | 58.792 | −24.897 | −9.591 | 1.00 | 43.37 | O |
| ATOM | 6911 | O | HOH | S | 225 | 33.558 | −1.910 | −11.130 | 1.00 | 42.43 | O |
| ATOM | 6912 | O | HOH | S | 226 | 37.380 | −1.739 | −10.425 | 1.00 | 28.36 | O |
| ATOM | 6913 | O | HOH | S | 227 | 42.144 | 4.658 | −14.005 | 1.00 | 37.17 | O |
| ATOM | 6914 | O | HOH | S | 228 | 35.259 | −32.067 | −4.230 | 1.00 | 26.38 | O |
| ATOM | 6915 | O | HOH | S | 229 | 27.054 | −29.024 | 23.868 | 1.00 | 31.26 | O |
| ATOM | 6916 | O | HOH | S | 230 | 28.025 | −24.836 | 21.982 | 1.00 | 41.72 | O |
| ATOM | 6917 | O | HOH | S | 231 | 29.718 | −21.619 | 22.035 | 1.00 | 26.91 | O |
| ATOM | 6918 | O | HOH | S | 232 | 31.922 | −21.147 | 18.078 | 1.00 | 25.15 | O |
| ATOM | 6919 | O | HOH | S | 233 | 30.736 | −19.391 | 13.985 | 1.00 | 20.11 | O |
| ATOM | 6920 | O | HOH | S | 234 | 23.449 | −20.932 | 8.267 | 1.00 | 19.81 | O |
| ATOM | 6921 | O | HOH | S | 235 | 38.734 | −43.341 | 10.590 | 1.00 | 25.00 | O |
| ATOM | 6922 | O | HOH | S | 236 | 35.059 | −42.083 | 3.191 | 1.00 | 22.87 | O |
| ATOM | 6923 | O | HOH | S | 237 | 30.442 | −38.963 | 7.221 | 1.00 | 29.13 | O |
| ATOM | 6924 | O | HOH | S | 238 | 29.017 | −38.789 | 14.741 | 1.00 | 19.77 | O |
| ATOM | 6925 | O | HOH | S | 239 | 32.641 | −53.110 | 23.474 | 1.00 | 21.18 | O |
| ATOM | 6926 | O | HOH | S | 240 | 46.012 | −30.442 | 22.963 | 1.00 | 23.93 | O |
| ATOM | 6927 | O | HOH | S | 241 | 46.834 | −32.884 | 24.530 | 1.00 | 26.05 | O |
| ATOM | 6928 | O | HOH | S | 242 | 50.948 | −53.714 | 11.342 | 1.00 | 30.41 | O |
| ATOM | 6929 | O | HOH | S | 243 | 56.368 | −58.407 | 17.911 | 1.00 | 17.43 | O |
| ATOM | 6930 | O | HOH | S | 244 | 56.278 | −50.345 | 21.311 | 1.00 | 36.95 | O |
| ATOM | 6931 | O | HOH | S | 245 | 52.774 | −49.094 | 23.573 | 1.00 | 21.50 | O |
| ATOM | 6932 | O | HOH | S | 246 | 36.665 | −46.272 | 26.081 | 1.00 | 31.14 | O |
| ATOM | 6933 | O | HOH | S | 247 | 40.189 | −52.429 | 20.575 | 1.00 | 18.07 | O |
| ATOM | 6934 | O | HOH | S | 248 | 42.474 | −52.031 | 14.703 | 1.00 | 20.16 | O |
| ATOM | 6935 | O | HOH | S | 249 | 46.036 | −48.258 | 12.637 | 1.00 | 18.22 | O |
| ATOM | 6936 | O | HOH | S | 250 | 12.287 | −37.590 | 13.822 | 1.00 | 23.79 | O |
| ATOM | 6937 | O | HOH | S | 251 | 25.804 | −24.495 | 13.714 | 1.00 | 24.23 | O |
| ATOM | 6938 | O | HOH | S | 252 | 19.221 | −8.215 | 4.588 | 1.00 | 28.77 | O |
| ATOM | 6939 | O | HOH | S | 253 | 8.487 | −26.744 | 4.946 | 1.00 | 50.99 | O |
| ATOM | 6940 | O | HOH | S | 254 | 54.430 | −21.902 | 28.337 | 1.00 | 25.10 | O |
| ATOM | 6941 | O | HOH | S | 255 | 47.197 | −12.422 | 25.707 | 1.00 | 29.48 | O |
| ATOM | 6942 | O | HOH | S | 256 | 43.978 | −25.660 | 9.756 | 1.00 | 24.11 | O |
| ATOM | 6943 | O | HOH | S | 257 | 62.807 | −20.268 | 12.441 | 1.00 | 18.09 | O |
| ATOM | 6944 | O | HOH | S | 258 | 49.879 | −38.733 | 24.568 | 1.00 | 21.95 | O |
| ATOM | 6945 | O | HOH | S | 259 | 39.125 | −1.658 | 15.166 | 1.00 | 25.66 | O |
| ATOM | 6946 | O | HOH | S | 260 | 29.981 | 0.051 | 20.524 | 1.00 | 15.11 | O |
| ATOM | 6947 | O | HOH | S | 261 | 12.290 | −18.170 | 21.698 | 1.00 | 14.38 | O |
| ATOM | 6948 | O | HOH | S | 262 | 29.656 | −11.387 | 14.988 | 1.00 | 16.32 | O |
| ATOM | 6949 | O | HOH | S | 263 | 67.995 | −35.613 | 9.255 | 1.00 | 20.89 | O |
| ATOM | 6950 | O | HOH | S | 264 | 68.864 | −37.145 | 12.647 | 1.00 | 21.09 | O |
| ATOM | 6951 | O | HOH | S | 265 | 57.849 | −39.211 | −21.703 | 1.00 | 22.40 | O |
| ATOM | 6952 | O | HOH | S | 266 | 34.489 | −29.321 | −24.048 | 1.00 | 25.19 | O |
| ATOM | 6953 | O | HOH | S | 267 | 18.846 | −38.700 | −10.227 | 1.00 | 22.90 | O |
| ATOM | 6954 | O | HOH | S | 268 | 49.097 | −35.728 | −10.757 | 1.00 | 26.77 | O |
| ATOM | 6955 | O | HOH | S | 269 | 52.988 | −35.652 | −15.540 | 1.00 | 30.41 | O |
| ATOM | 6956 | O | HOH | S | 270 | 59.414 | −21.695 | 3.982 | 1.00 | 17.25 | O |
| ATOM | 6957 | O | HOH | S | 271 | 32.772 | −5.429 | −14.234 | 1.00 | 22.42 | O |
| ATOM | 6958 | O | HOH | S | 272 | 30.235 | −23.166 | −1.299 | 1.00 | 25.69 | O |
| ATOM | 6959 | O | HOH | S | 273 | 47.565 | −6.784 | −15.947 | 1.00 | 20.80 | O |
| ATOM | 6960 | O | HOH | S | 274 | 58.329 | −9.425 | −24.535 | 1.00 | 25.87 | O |
| ATOM | 6961 | O | HOH | S | 275 | 52.062 | −17.724 | −14.570 | 1.00 | 21.54 | O |
| ATOM | 6962 | O | HOH | S | 276 | 45.063 | −11.846 | −8.590 | 1.00 | 25.53 | O |
| ATOM | 6963 | O | HOH | S | 277 | 55.791 | −9.606 | −8.612 | 1.00 | 18.20 | O |
| ATOM | 6964 | O | HOH | S | 278 | 57.399 | −17.645 | −6.569 | 1.00 | 22.14 | O |
| ATOM | 6965 | O | HOH | S | 279 | 80.209 | −5.449 | −18.368 | 1.00 | 19.59 | O |
| ATOM | 6966 | O | HOH | S | 280 | 42.002 | −5.777 | 11.842 | 1.00 | 45.47 | O |
| ATOM | 6967 | O | HOH | S | 281 | 14.299 | −7.223 | 21.824 | 1.00 | 15.49 | O |
| ATOM | 6968 | O | HOH | S | 282 | 14.103 | −30.114 | −9.777 | 1.00 | 22.76 | O |
| ATOM | 6969 | O | HOH | S | 283 | 13.150 | −30.932 | −14.205 | 1.00 | 22.00 | O |
| ATOM | 6970 | O | HOH | S | 284 | 11.490 | −29.073 | −15.278 | 1.00 | 32.12 | O |
| ATOM | 6971 | O7 | LPA | A | 1 | 59.990 | −37.052 | 14.947 | 1.00 | 23.28 | O |
| ATOM | 6972 | C4 | LPA | A | 1 | 59.901 | −38.205 | 14.485 | 1.00 | 23.50 | C |
| ATOM | 6973 | O6 | LPA | A | 1 | 60.488 | −38.446 | 13.195 | 1.00 | 23.82 | O |
| ATOM | 6974 | C3 | LPA | A | 1 | 61.265 | −37.438 | 12.571 | 1.00 | 21.98 | C |
| ATOM | 6975 | C2 | LPA | A | 1 | 61.834 | −38.145 | 11.353 | 1.00 | 22.15 | C |
| ATOM | 6976 | O5 | LPA | A | 1 | 60.818 | −38.270 | 10.345 | 1.00 | 22.67 | O |
| ATOM | 6977 | C1 | LPA | A | 1 | 63.028 | −37.367 | 10.813 | 1.00 | 20.03 | C |
| ATOM | 6978 | O4 | LPA | A | 1 | 64.047 | −37.374 | 11.806 | 1.00 | 22.58 | O |
| ATOM | 6979 | P1 | LPA | A | 1 | 65.352 | −36.465 | 11.581 | 1.00 | 24.50 | P |
| ATOM | 6980 | O3 | LPA | A | 1 | 65.949 | −36.942 | 10.296 | 1.00 | 24.47 | O |
| ATOM | 6981 | O2 | LPA | A | 1 | 64.827 | −35.070 | 11.554 | 1.00 | 23.87 | O |
| ATOM | 6982 | O1 | LPA | A | 1 | 66.199 | −36.779 | 12.801 | 1.00 | 24.24 | O |
| ATOM | 6983 | C5 | LPA | A | 1 | 59.206 | −39.353 | 15.188 | 1.00 | 24.44 | C |
| ATOM | 6984 | C6 | LPA | A | 1 | 59.571 | −40.710 | 14.547 | 1.00 | 22.84 | C |
| ATOM | 6985 | C7 | LPA | A | 1 | 59.100 | −41.914 | 15.376 | 1.00 | 23.22 | C |
| ATOM | 6986 | C8 | LPA | A | 1 | 59.758 | −43.187 | 14.821 | 1.00 | 24.86 | C |
| ATOM | 6987 | C9 | LPA | A | 1 | 59.062 | −44.490 | 15.234 | 1.00 | 25.39 | C |
| ATOM | 6988 | C10 | LPA | A | 1 | 59.487 | −45.590 | 14.242 | 1.00 | 28.40 | C |
| ATOM | 6989 | C11 | LPA | A | 1 | 58.815 | −46.933 | 14.536 | 1.00 | 31.40 | C |
| ATOM | 6990 | C12 | LPA | A | 1 | 59.337 | −48.039 | 13.604 | 1.00 | 33.63 | C |
| ATOM | 6991 | C13 | LPA | A | 1 | 60.806 | −48.348 | 13.868 | 1.00 | 36.13 | C |
| ATOM | 6992 | C14 | LPA | A | 1 | 61.139 | −49.819 | 13.653 | 1.00 | 38.39 | C |
| ATOM | 6993 | C15 | LPA | A | 1 | 62.475 | −49.978 | 12.950 | 1.00 | 40.56 | C |
| ATOM | 6994 | C16 | LPA | A | 1 | 63.581 | −50.373 | 13.918 | 1.00 | 41.92 | C |
| ATOM | 6995 | C17 | LPA | A | 1 | 64.594 | −51.277 | 13.248 | 1.00 | 41.48 | C |
| ATOM | 6996 | O7 | LPA | B | 1 | 21.835 | −30.872 | −15.472 | 1.00 | 27.70 | O |
| ATOM | 6997 | C4 | LPA | B | 1 | 21.784 | −32.093 | −15.401 | 1.00 | 26.65 | C |
| ATOM | 6998 | O6 | LPA | B | 1 | 21.130 | −32.650 | −14.246 | 1.00 | 26.61 | O |
| ATOM | 6999 | C3 | LPA | B | 1 | 20.632 | −31.821 | −13.213 | 1.00 | 25.07 | C |
| ATOM | 7000 | C2 | LPA | B | 1 | 19.816 | −32.738 | −12.323 | 1.00 | 28.37 | C |
| ATOM | 7001 | O5 | LPA | B | 1 | 20.722 | −33.330 | −11.397 | 1.00 | 26.33 | O |
| ATOM | 7002 | C1 | LPA | B | 1 | 18.752 | −31.908 | −11.618 | 1.00 | 27.76 | C |
| ATOM | 7003 | O4 | LPA | B | 1 | 17.811 | −31.505 | −12.620 | 1.00 | 28.14 | O |
| ATOM | 7004 | P1 | LPA | B | 1 | 16.553 | −30.560 | −12.280 | 1.00 | 30.48 | P |
| ATOM | 7005 | O3 | LPA | B | 1 | 15.810 | −30.523 | −13.582 | 1.00 | 31.66 | O |
| ATOM | 7006 | O2 | LPA | B | 1 | 15.876 | −31.252 | −11.120 | 1.00 | 30.11 | O |
| ATOM | 7007 | O1 | LPA | B | 1 | 17.195 | −29.242 | −11.888 | 1.00 | 31.01 | O |
| ATOM | 7008 | C5 | LPA | B | 1 | 22.348 | −32.997 | −16.477 | 1.00 | 27.56 | C |
| ATOM | 7009 | C6 | LPA | B | 1 | 21.637 | −34.357 | −16.477 | 1.00 | 28.92 | C |
| ATOM | 7010 | C7 | LPA | B | 1 | 22.181 | −35.300 | −17.549 | 1.00 | 30.31 | C |
| ATOM | 7011 | C8 | LPA | B | 1 | 21.403 | −36.619 | −17.516 | 1.00 | 33.45 | C |
| ATOM | 7012 | C9 | LPA | B | 1 | 22.071 | −37.741 | −18.298 | 1.00 | 35.30 | C |
| ATOM | 7013 | C10 | LPA | B | 1 | 21.692 | −39.080 | −17.644 | 1.00 | 37.59 | C |
| ATOM | 7014 | C11 | LPA | B | 1 | 21.762 | −40.238 | −18.620 | 1.00 | 39.44 | C |
| ATOM | 7015 | C12 | LPA | B | 1 | 20.774 | −41.355 | −18.272 | 1.00 | 38.73 | C |
| ATOM | 7016 | C13 | LPA | B | 1 | 21.474 | −42.451 | −17.481 | 1.00 | 39.29 | C |
| ATOM | 7017 | C14 | LPA | B | 1 | 20.496 | −43.514 | −16.988 | 1.00 | 40.41 | C |
| ATOM | 7018 | C15 | LPA | B | 1 | 20.007 | −44.429 | −18.098 | 1.00 | 41.23 | C |
| ATOM | 7019 | C16 | LPA | B | 1 | 19.815 | −45.858 | −17.583 | 1.00 | 42.16 | C |
| ATOM | 7020 | C17 | LPA | B | 1 | 18.362 | −46.251 | −17.719 | 1.00 | 41.41 | C |

Structure Solution, Model Building, and Refinement: Fab:LPA(18:2) Complex. Merged scalepack output were converted to MTZ format using scalepack2mtz. The CCP4 suite: programs for protein crystallography (1994). Acta Crystallogr. D. Biol. Crystallogr. 50:760-763. Molecular replacement was carried out in phaser using the 14:0 complex as a starting model. McCoy, A. J. et al. (2007) J. Appl Crystallogr 40:658:674. Further refinement by maximum likelihood methods was run in REFMAC5. Murshudov G N, Vagin A A, Dodson E J (1997) Acta Crystallogr. D. Biol. Crystallogr. 53: 240-255. The model was rebuilt in the program Coot. Emsley, P. et al., (2010) Acta Crystallogr. D. Biol. Crystallogr. 66:486-501. The model was then further built in the program PHENIX. Adams, P. D. et al. (2010) Acta Crystallogr. D. Biol. Crystallogr. 66:213:221. Coordinates for lysophosphatidic acid were prepared using the PRODRG server. Schuttelkopf A. W, van Aalten DM (2004) Acta Crystallogr. D. Biol. Crystallogr. 60:1355-1363. A library file was prepared via Monomer Library Sketcher. The CCP4 suite: programs for protein crystallography (1994). Acta Crystallogr. D. Biol. Crystallogr. 50:760-763. Stereochemical analysis and final adjustments to the model were directed by MOLPROBITY. Davis I W et al., (2007) Nucl. Acids. Res. 35:W375-383. Final adjustments were made in Coot. Emsley, P. et al., (2010) Acta Crystallogr. D. Biol. Crystallogr. 66:486-501.

Figures were created using PyMOL. Delano WL (2002) The PyMOL Molecular Graphics System (DeLano Scientific, San Carlos Calif.). Schematic diagrams were createdusing LIGPLOT. Wallace A C et al (1995) Protein Eng. 8:127-134.

The coordinates for the Fab:LPA(18:2) crystal are provided as Table 21, below.

TABLE 21
Fab:LPA(18:2) co-crystal x-ray coordinates at 2.51 Å resolution

| REMARK | 3 | REFINEMENT. | |
|---|---|---|---|
| REMARK | 3 | PROGRAM: REFMAC 5.5.0109 | |
| REMARK | 3 | REFINEMENT TARGET: MAXIMUM LIKELIHOOD | |
| REMARK | 3 | DATA USED IN REFINEMENT. | |
| REMARK | 3 | RESOLUTION RANGE HIGH (ANGSTROMS): | 2.51 |
| REMARK | 3 | RESOLUTION RANGE LOW (ANGSTROMS): | 91.57 |
| REMARK | 3 | DATA CUTOFF (SIGMA(F)): NONE | |

| REMARK | 3 | COMPLETENESS FOR RANGE (%): | 99.24 |
|---|---|---|---|
| REMARK | 3 | NUMBER OF REFLECTIONS: | 33177 |
| REMARK | 3 | FIT TO DATA USED IN REFINEMENT. | |
| REMARK | 3 | CROSS-VALIDATION METHOD: | THROUGHOUT |
| REMARK | 3 | FREE R VALUE TEST SET SELECTION: | RANDOM |

| | | | |
|---|---|---|---|
| REMARK | 3 | R VALUE (WORKING + TEST SET): | 0.21754 |

| | | | |
|---|---|---|---|
| REMARK | 3 | R VALUE (WORKING SET): | 0.21493 |

| | | | |
|---|---|---|---|
| REMARK | 3 | FREE R VALUE: | 0.26611 |

| | | | |
|---|---|---|---|
| REMARK | 3 | FREE R VALUE TEST SET SIZE (%): | 5.0 |
| REMARK | 3 | FREE R VALUE TEST SET COUNT: | 1754 |

| REMARK | 3 | FIT IN THE HIGHEST RESOLUTION BIN. | |
|---|---|---|---|
| REMARK | 3 | TOTAL NUMBER OF BINS USED: | 20 |
| REMARK | 3 | BIN RESOLUTION RANGE HIGH: | 2.510 |
| REMARK | 3 | BIN RESOLUTION RANGE LOW: | 2.575 |

| | | | |
|---|---|---|---|
| REMARK | 3 | REFLECTION IN BIN (WORKING SET): | 2200 |

| | | | |
|---|---|---|---|
| REMARK | 3 | BIN COMPLETENESS (WORKING + TEST) (%): | 89.95 |

| | | | |
|---|---|---|---|
| REMARK | 3 | BIN R VALUE (WORKING SET): | 0.272 |
| REMARK | 3 | BIN FREE R VALUE SET COUNT: | 118 |

| | | | |
|---|---|---|---|
| REMARK | 3 | BIN FREE R VALUE: | 0.381 |

| REMARK | 3 | NUMBER OF NON-HYDROGEN ATOMS USED IN REFINEMENT. | |
|---|---|---|---|
| REMARK | 3 | ALL ATOMS: | 6841 |
| REMARK | 3 | B VALUES. | |

| | | | | |
|---|---|---|---|---|
| REMARK | 3 | FROM WILSON PLOT | (A**2): | NULL |

| | | | | |
|---|---|---|---|---|
| REMARK | 3 | MEAN B VALUE | (OVERALL, A**2): | 27.937 |

| REMARK | 3 | OVERALL ANISOTROPIC B VALUE. | |
|---|---|---|---|
| REMARK | 3 | B11 (A**2): | 0.33 |
| REMARK | 3 | B22 (A**2): | −0.02 |
| REMARK | 3 | B33 (A**2): | −0.31 |
| REMARK | 3 | B12 (A**2): | 0.00 |
| REMARK | 3 | B13 (A**2): | 0.00 |

| REMARK | 3 | ESTIMATED OVERALL COORDINATE ERROR. | |
|---|---|---|---|
| REMARK | 3 | ESU BASED ON R VALUE (A): 0.597 | |
| REMARK | 3 | ESU BASED ON FREE R VALUE (A): 0.309 | |
| REMARK | 3 | ESU BASED ON MAXIMUM LIKELIHOOD (A): 0.225 | |
| REMARK | 3 | ESU FOR B VALUES BASED ON MAXIMUM LIKELIHOOD (A**2): 10.034 | |
| REMARK | 3 | CORRELATION COEFFICIENTS. | |
| REMARK | 3 | CORRELATION COEFFICIENT FO-FC: | 0.922 |
| REMARK | 3 | CORRELATION COEFFICIENT FO-FC FREE: | 0.873 |

| | | | |
|---|---|---|---|
| REMARK | 3 | RMS DEVIATIONS FROM IDEAL VALUES | COUNT RMS WEIGHT |

| | | | |
|---|---|---|---|
| REMARK | 3 | BOND LENGTHS REFINED ATOMS (A): | 6918 ; 0.010 ; 0.022 |

| | | | |
|---|---|---|---|
| REMARK | 3 | BOND ANGLES REFINED ATOMS (DEGREES): | 9398 ; 1.272 ; 1.958 |

| | | | |
|---|---|---|---|
| REMARK | 3 | TORSION ANGLES, PERIOD 1 (DEGREES): | 868 ; 6.423 ; 5.000 |
| REMARK | 3 | TORSION ANGLES, PERIOD 2 (DEGREES): | 276 ; 36.606 ; 24.710 |
| REMARK | 3 | TORSION ANGLES, PERIOD 3 (DEGREES): | 1088 ; 15.861 ; 15.000 |
| REMARK | 3 | TORSION ANGLES, PERIOD 4 (DEGREES): | 18 ; 21.529 ; 15.000 |

| | | | |
|---|---|---|---|
| REMARK | 3 | CHIRAL-CENTER RESTRAINTS (A**3): | 1034 ; 0.077 ; 0.200 |

| | | | |
|---|---|---|---|
| REMARK | 3 | GENERAL PLANES REFINED ATOMS (A): | 5218 ; 0.005 ; 0.021 |
| REMARK | 3 | | |

| | | | |
|---|---|---|---|
| REMARK | 3 | ISOTROPIC THERMAL FACTOR RESTRAINTS. | COUNT RMS WEIGHT |

| | | | |
|---|---|---|---|
| REMARK | 3 | MAIN-CHAIN BOND REFINED ATOMS (A**2): | 4340 ; 0.563 ; 1.500 |
| REMARK | 3 | MAIN-CHAIN ANGLE REFINED ATOMS (A**2): | 7020 ; 1.081 ; 2.000 |
| REMARK | 3 | SIDE-CHAIN BOND REFINED ATOMS (A**2): | 2578 ; 1.444 ; 3.000 |
| REMARK | 3 | SIDE-CHAIN ANGLE REFINED ATOMS (A**2): | 2378 ; 2.529 ; 4.500 |

| REMARK | 3 | NCS RESTRAINTS STATISTICS |
|---|---|---|
| REMARK | 3 | NUMBER OF NCS GROUPS: NULL |
| REMARK | 3 | TWIN DETAILS |
| REMARK | 3 | NUMBER OF TWIN DOMAINS: NULL |
| REMARK | 3 | TLS DETAILS |
| REMARK | 3 | NUMBER OF TLS GROUPS: NULL |
| REMARK | 3 | BULK SOLVENT MODELLING. |
| REMARK | 3 | METHOD USED: MASK |
| REMARK | 3 | PARAMETERS FOR MASK CALCULATION |
| REMARK | 3 | VDW PROBE RADIUS: 1.40 |
| REMARK | 3 | ION PROBE RADIUS: 0.80 |
| REMARK | 3 | SHRINKAGE RADIUS: 0.80 |
| REMARK | 3 | OTHER REFINEMENT REMARKS: |
| REMARK | 3 | HYDROGENS HAVE BEEN ADDED IN THE RIDING POSITIONS |
| SSBOND | 1 | CYS H 22 CYS H 92 |
| SSBOND | 2 | CYS H 140 CYS H 196 |
| SSBOND | 3 | CYS L 23 CYS L 88 |
| SSBOND | 4 | CYS L 134 CYS L 194 |
| SSBOND | 5 | CYS I 22 CYS I 92 |
| SSBOND | 6 | CYS I 140 CYS I 196 |
| SSBOND | 7 | CYS M 23 CYS M 88 |
| SSBOND | 8 | CYS M 134 CYS M 194 |
| LINKR | | SER H 128 GLY H 133 gap |
| CISPEP | 1 PHE H 146 PRO H 147 0.00 | |
| CISPEP | 2 GLU H 148 PRO H 149 0.00 | |
| CISPEP | 3 THR L 7 PRO L 8 0.00 | |
| CISPEP | 4 PHE L 94 PRO L 95 0.00 | |
| CISPEP | 5 TYR L 140 PRO L 141 0.00 | |
| LINKR | | ALA H 137 VAL H 142 gap |
| LINKR | | SER I 128 GLY I 133 gap |
| CISPEP | 6 PHE I 146 PRO I 147 0.00 | |
| CISPEP | 7 GLU I 148 PRO I 149 0.00 | |
| LINKR | | ALA I 137 VAL I 142 gap |

| | |
|---|---|
| CISPEP | 8 THR M 7 PRO M 8 0.00 |
| CISPEP | 9 PHE M 94 PRO M 95 0.00 |
| CISPEP | 10 TYR M 140 PRO M 141 0.00 |
| CRYST1 | 86.980 183.131 127.498 90.00 90.00 90.00 C 2 2 21 |
| SCALE1 | 0.011497 0.000000 0.000000 0.00000 |
| SCALE2 | 0.000000 0.005461 0.000000 0.00000 |
| SCALE3 | 0.000000 0.000000 0.007843 0.00000 |

ATOMS and Details

| | | | | | | | | | | | |
|---|---|---|---|---|---|---|---|---|---|---|---|
| ATOM | 1 | N | GLU | H | 1 | −7.989 | 36.125 | −4.692 | 1.00 | 44.78 | N |
| ATOM | 2 | CA | GLU | H | 1 | −6.980 | 35.984 | −3.602 | 1.00 | 44.89 | C |
| ATOM | 3 | CB | GLU | H | 1 | −5.700 | 35.299 | −4.121 | 1.00 | 45.31 | C |
| ATOM | 4 | CG | GLU | H | 1 | −5.857 | 33.828 | −4.546 | 1.00 | 46.73 | C |
| ATOM | 5 | CD | GLU | H | 1 | −5.632 | 32.830 | −3.405 | 1.00 | 49.19 | C |
| ATOM | 6 | OE1 | GLU | H | 1 | −5.588 | 31.606 | −3.682 | 1.00 | 49.13 | O |
| ATOM | 7 | OE2 | GLU | H | 1 | −5.499 | 33.258 | −2.234 | 1.00 | 50.02 | O |
| ATOM | 8 | C | GLU | H | 1 | −6.635 | 37.352 | −2.991 | 1.00 | 44.25 | C |
| ATOM | 9 | O | GLU | H | 1 | −6.520 | 38.353 | −3.705 | 1.00 | 44.57 | O |
| ATOM | 10 | N | VAL | H | 2 | −6.498 | 37.383 | −1.666 | 1.00 | 42.99 | N |
| ATOM | 11 | CA | VAL | H | 2 | −5.919 | 38.524 | −0.957 | 1.00 | 41.44 | C |
| ATOM | 12 | CB | VAL | H | 2 | −6.943 | 39.167 | 0.029 | 1.00 | 41.75 | C |
| ATOM | 13 | CG1 | VAL | H | 2 | −6.252 | 39.835 | 1.234 | 1.00 | 41.98 | C |
| ATOM | 14 | CG2 | VAL | H | 2 | −7.836 | 40.172 | −0.708 | 1.00 | 41.80 | C |
| ATOM | 15 | C | VAL | H | 2 | −4.647 | 38.027 | −0.254 | 1.00 | 40.12 | C |
| ATOM | 16 | O | VAL | H | 2 | −4.559 | 36.864 | 0.154 | 1.00 | 40.64 | O |
| ATOM | 17 | N | GLN | H | 3 | −3.658 | 38.900 | −0.133 | 1.00 | 37.87 | N |
| ATOM | 18 | CA | GLN | H | 3 | −2.371 | 38.514 | 0.422 | 1.00 | 35.59 | C |
| ATOM | 19 | CB | GLN | H | 3 | −1.387 | 38.225 | −0.708 | 1.00 | 35.77 | C |
| ATOM | 20 | CG | GLN | H | 3 | −0.135 | 37.507 | −0.265 | 1.00 | 37.47 | C |
| ATOM | 21 | CD | GLN | H | 3 | 0.733 | 37.091 | −1.424 | 1.00 | 40.29 | C |
| ATOM | 22 | OE1 | GLN | H | 3 | 0.305 | 37.118 | −2.585 | 1.00 | 41.79 | O |
| ATOM | 23 | NE2 | GLN | H | 3 | 1.965 | 36.696 | −1.121 | 1.00 | 40.85 | N |
| ATOM | 24 | C | GLN | H | 3 | −1.832 | 39.614 | 1.319 | 1.00 | 33.58 | C |
| ATOM | 25 | O | GLN | H | 3 | −2.012 | 40.803 | 1.045 | 1.00 | 33.77 | O |
| ATOM | 26 | N | LEU | H | 4 | −1.173 | 39.208 | 2.390 | 1.00 | 30.88 | N |
| ATOM | 27 | CA | LEU | H | 4 | −0.605 | 40.141 | 3.325 | 1.00 | 28.57 | C |
| ATOM | 28 | CB | LEU | H | 4 | −1.213 | 39.942 | 4.705 | 1.00 | 28.34 | C |
| ATOM | 29 | CG | LEU | H | 4 | −2.718 | 40.140 | 4.824 | 1.00 | 27.44 | C |
| ATOM | 30 | CD1 | LEU | H | 4 | −3.185 | 39.816 | 6.239 | 1.00 | 26.99 | C |
| ATOM | 31 | CD2 | LEU | H | 4 | −3.073 | 41.548 | 4.466 | 1.00 | 27.18 | C |
| ATOM | 32 | C | LEU | H | 4 | 0.891 | 39.925 | 3.378 | 1.00 | 27.48 | C |
| ATOM | 33 | O | LEU | H | 4 | 1.353 | 38.811 | 3.630 | 1.00 | 27.20 | O |
| ATOM | 34 | N | VAL | H | 5 | 1.650 | 40.988 | 3.128 | 1.00 | 26.08 | N |
| ATOM | 35 | CA | VAL | H | 5 | 3.105 | 40.899 | 3.133 | 1.00 | 24.76 | C |
| ATOM | 36 | CB | VAL | H | 5 | 3.714 | 41.285 | 1.765 | 1.00 | 24.92 | C |
| ATOM | 37 | CG1 | VAL | H | 5 | 5.220 | 41.104 | 1.790 | 1.00 | 24.51 | C |
| ATOM | 38 | CG2 | VAL | H | 5 | 3.098 | 40.462 | 0.648 | 1.00 | 23.05 | C |
| ATOM | 39 | C | VAL | H | 5 | 3.676 | 41.788 | 4.234 | 1.00 | 24.46 | C |
| ATOM | 40 | O | VAL | H | 5 | 3.418 | 43.000 | 4.276 | 1.00 | 23.96 | O |
| ATOM | 41 | N | GLN | H | 6 | 4.437 | 41.165 | 5.130 | 1.00 | 23.70 | N |
| ATOM | 42 | CA | GLN | H | 6 | 5.031 | 41.856 | 6.261 | 1.00 | 23.35 | C |
| ATOM | 43 | CB | GLN | H | 6 | 4.902 | 41.001 | 7.510 | 1.00 | 23.33 | C |
| ATOM | 44 | CG | GLN | H | 6 | 3.463 | 40.830 | 7.976 | 1.00 | 23.23 | C |
| ATOM | 45 | CD | GLN | H | 6 | 3.341 | 39.933 | 9.190 | 1.00 | 22.25 | C |
| ATOM | 46 | OE1 | GLN | H | 6 | 2.826 | 38.828 | 9.097 | 1.00 | 22.58 | O |
| ATOM | 47 | NE2 | GLN | H | 6 | 3.815 | 40.403 | 10.328 | 1.00 | 21.43 | N |
| ATOM | 48 | C | GLN | H | 6 | 6.487 | 42.206 | 6.003 | 1.00 | 23.15 | C |
| ATOM | 49 | O | GLN | H | 6 | 7.144 | 41.559 | 5.199 | 1.00 | 23.36 | O |
| ATOM | 50 | N | SER | H | 7 | 6.986 | 43.240 | 6.672 | 1.00 | 22.97 | N |
| ATOM | 51 | CA | SER | H | 7 | 8.389 | 43.659 | 6.507 | 1.00 | 22.80 | C |
| ATOM | 52 | CB | SER | H | 7 | 8.627 | 45.066 | 7.095 | 1.00 | 22.56 | C |
| ATOM | 53 | OG | SER | H | 7 | 8.200 | 45.152 | 8.445 | 1.00 | 22.16 | O |
| ATOM | 54 | C | SER | H | 7 | 9.362 | 42.638 | 7.105 | 1.00 | 22.67 | C |
| ATOM | 55 | O | SER | H | 7 | 8.947 | 41.707 | 7.812 | 1.00 | 22.51 | O |
| ATOM | 56 | N | GLY | H | 8 | 10.647 | 42.822 | 6.817 | 1.00 | 22.64 | N |
| ATOM | 57 | CA | GLY | H | 8 | 11.689 | 41.874 | 7.207 | 1.00 | 23.05 | C |
| ATOM | 58 | C | GLY | H | 8 | 11.998 | 41.746 | 8.692 | 1.00 | 23.30 | C |
| ATOM | 59 | O | GLY | H | 8 | 11.629 | 42.606 | 9.505 | 1.00 | 22.89 | O |
| ATOM | 60 | N | ALA | H | 9 | 12.699 | 40.666 | 9.032 | 1.00 | 23.65 | N |
| ATOM | 61 | CA | ALA | H | 9 | 13.018 | 40.341 | 10.415 | 1.00 | 24.24 | C |
| ATOM | 62 | CB | ALA | H | 9 | 13.742 | 39.008 | 10.499 | 1.00 | 24.23 | C |
| ATOM | 63 | C | ALA | H | 9 | 13.834 | 41.442 | 11.072 | 1.00 | 24.53 | C |
| ATOM | 64 | O | ALA | H | 9 | 14.722 | 42.019 | 10.445 | 1.00 | 24.50 | O |
| ATOM | 65 | N | GLU | H | 10 | 13.496 | 41.723 | 12.333 | 1.00 | 24.94 | N |
| ATOM | 66 | CA | GLU | H | 10 | 14.040 | 42.842 | 13.114 | 1.00 | 25.24 | C |
| ATOM | 67 | CB | GLU | H | 10 | 12.900 | 43.640 | 13.762 | 1.00 | 25.14 | C |
| ATOM | 68 | CG | GLU | H | 10 | 11.940 | 44.353 | 12.791 | 1.00 | 26.51 | C |
| ATOM | 69 | CD | GLU | H | 10 | 12.345 | 45.788 | 12.470 | 1.00 | 27.21 | C |
| ATOM | 70 | OE1 | GLU | H | 10 | 12.247 | 46.668 | 13.346 | 1.00 | 29.63 | O |
| ATOM | 71 | OE2 | GLU | H | 10 | 12.742 | 46.052 | 11.332 | 1.00 | 28.18 | O |
| ATOM | 72 | C | GLU | H | 10 | 14.941 | 42.309 | 14.225 | 1.00 | 25.15 | C |
| ATOM | 73 | O | GLU | H | 10 | 14.669 | 41.250 | 14.800 | 1.00 | 25.19 | O |
| ATOM | 74 | N | VAL | H | 11 | 16.005 | 43.046 | 14.534 | 1.00 | 24.97 | N |
| ATOM | 75 | CA | VAL | H | 11 | 16.833 | 42.745 | 15.704 | 1.00 | 24.81 | C |
| ATOM | 76 | CB | VAL | H | 11 | 18.214 | 42.137 | 15.333 | 1.00 | 24.69 | C |
| ATOM | 77 | CG1 | VAL | H | 11 | 18.996 | 41.797 | 16.590 | 1.00 | 24.20 | C |
| ATOM | 78 | CG2 | VAL | H | 11 | 18.059 | 40.900 | 14.443 | 1.00 | 23.20 | C |
| ATOM | 79 | C | VAL | H | 11 | 17.020 | 44.042 | 16.469 | 1.00 | 25.38 | C |
| ATOM | 80 | O | VAL | H | 11 | 17.530 | 45.019 | 15.915 | 1.00 | 25.54 | O |
| ATOM | 81 | N | LYS | H | 12 | 16.593 | 44.057 | 17.732 | 1.00 | 25.65 | N |
| ATOM | 82 | CA | LYS | H | 12 | 16.608 | 45.279 | 18.530 | 1.00 | 25.92 | C |
| ATOM | 83 | CB | LYS | H | 12 | 15.190 | 45.843 | 18.695 | 1.00 | 26.07 | C |
| ATOM | 84 | CG | LYS | H | 12 | 14.512 | 46.263 | 17.413 | 1.00 | 26.62 | C |
| ATOM | 85 | CD | LYS | H | 12 | 14.824 | 47.692 | 17.039 | 1.00 | 27.90 | C |
| ATOM | 86 | CE | LYS | H | 12 | 14.737 | 47.868 | 15.533 | 1.00 | 29.51 | C |
| ATOM | 87 | NZ | LYS | H | 12 | 14.734 | 49.306 | 15.168 | 1.00 | 30.65 | N |
| ATOM | 88 | C | LYS | H | 12 | 17.210 | 45.034 | 19.900 | 1.00 | 26.04 | C |
| ATOM | 89 | O | LYS | H | 12 | 17.348 | 43.893 | 20.324 | 1.00 | 26.16 | O |
| ATOM | 90 | N | LYS | H | 13 | 17.556 | 46.122 | 20.585 | 1.00 | 25.98 | N |
| ATOM | 91 | CA | LYS | H | 13 | 18.077 | 46.070 | 21.940 | 1.00 | 26.05 | C |
| ATOM | 92 | CB | LYS | H | 13 | 19.227 | 47.067 | 22.094 | 1.00 | 26.63 | C |
| ATOM | 93 | CG | LYS | H | 13 | 20.569 | 46.484 | 21.788 | 1.00 | 27.95 | C |
| ATOM | 94 | CD | LYS | H | 13 | 21.590 | 47.549 | 21.493 | 1.00 | 31.33 | C |
| ATOM | 95 | CE | LYS | H | 13 | 22.923 | 46.906 | 21.082 | 1.00 | 33.29 | C |
| ATOM | 96 | NZ | LYS | H | 13 | 22.760 | 45.773 | 20.095 | 1.00 | 33.37 | N |
| ATOM | 97 | C | LYS | H | 13 | 16.969 | 46.412 | 22.924 | 1.00 | 25.68 | C |
| ATOM | 98 | O | LYS | H | 13 | 16.017 | 47.112 | 22.564 | 1.00 | 25.57 | O |
| ATOM | 99 | N | PRO | H | 14 | 17.072 | 45.909 | 24.168 | 1.00 | 25.22 | N |
| ATOM | 100 | CA | PRO | H | 14 | 16.075 | 46.267 | 25.165 | 1.00 | 25.25 | C |
| ATOM | 101 | CB | PRO | H | 14 | 16.645 | 45.669 | 26.448 | 1.00 | 25.23 | C |
| ATOM | 102 | CG | PRO | H | 14 | 17.440 | 44.513 | 25.982 | 1.00 | 24.75 | C |
| ATOM | 103 | CD | PRO | H | 14 | 18.008 | 44.886 | 24.666 | 1.00 | 25.05 | C |
| ATOM | 104 | C | PRO | H | 14 | 15.960 | 47.785 | 25.278 | 1.00 | 25.34 | C |
| ATOM | 105 | O | PRO | H | 14 | 16.978 | 48.477 | 25.227 | 1.00 | 25.29 | O |
| ATOM | 106 | N | GLY | H | 15 | 14.728 | 48.285 | 25.378 | 1.00 | 25.52 | N |
| ATOM | 107 | CA | GLY | H | 15 | 14.452 | 49.719 | 25.523 | 1.00 | 25.82 | C |
| ATOM | 108 | C | GLY | H | 15 | 14.208 | 50.444 | 24.210 | 1.00 | 26.14 | C |
| ATOM | 109 | O | GLY | H | 15 | 13.629 | 51.546 | 24.192 | 1.00 | 26.45 | O |
| ATOM | 110 | N | GLU | H | 16 | 14.654 | 49.834 | 23.115 | 1.00 | 25.74 | N |
| ATOM | 111 | CA | GLU | H | 16 | 14.442 | 50.382 | 21.785 | 1.00 | 25.97 | C |
| ATOM | 112 | CB | GLU | H | 16 | 15.396 | 49.734 | 20.767 | 1.00 | 25.91 | C |
| ATOM | 113 | CG | GLU | H | 16 | 16.816 | 50.235 | 20.917 | 1.00 | 27.93 | C |
| ATOM | 114 | CD | GLU | H | 16 | 17.829 | 49.616 | 19.960 | 1.00 | 31.38 | C |
| ATOM | 115 | OE1 | GLU | H | 16 | 19.017 | 49.997 | 20.059 | 1.00 | 32.93 | O |
| ATOM | 116 | OE2 | GLU | H | 16 | 17.466 | 48.770 | 19.114 | 1.00 | 32.96 | O |
| ATOM | 117 | C | GLU | H | 16 | 12.978 | 50.236 | 21.356 | 1.00 | 25.73 | C |
| ATOM | 118 | O | GLU | H | 16 | 12.142 | 49.734 | 22.106 | 1.00 | 25.94 | O |
| ATOM | 119 | N | SER | H | 17 | 12.669 | 50.698 | 20.153 | 1.00 | 25.50 | N |
| ATOM | 120 | CA | SER | H | 17 | 11.316 | 50.601 | 19.628 | 1.00 | 25.21 | C |
| ATOM | 121 | CB | SER | H | 17 | 10.573 | 51.949 | 19.738 | 1.00 | 25.06 | C |
| ATOM | 122 | OG | SER | H | 17 | 11.075 | 52.898 | 18.810 | 1.00 | 25.90 | O |
| ATOM | 123 | C | SER | H | 17 | 11.335 | 50.069 | 18.193 | 1.00 | 24.72 | C |
| ATOM | 124 | O | SER | H | 17 | 12.317 | 50.238 | 17.464 | 1.00 | 24.97 | O |
| ATOM | 125 | N | LEU | H | 18 | 10.259 | 49.396 | 17.807 | 1.00 | 24.22 | N |
| ATOM | 126 | CA | LEU | H | 18 | 10.159 | 48.880 | 16.457 | 1.00 | 23.81 | C |
| ATOM | 127 | CB | LEU | H | 18 | 10.198 | 47.344 | 16.460 | 1.00 | 23.96 | C |
| ATOM | 128 | CG | LEU | H | 18 | 9.087 | 46.529 | 17.136 | 1.00 | 23.45 | C |
| ATOM | 129 | CD1 | LEU | H | 18 | 7.769 | 46.546 | 16.359 | 1.00 | 21.92 | C |
| ATOM | 130 | CD2 | LEU | H | 18 | 9.570 | 45.114 | 17.279 | 1.00 | 21.78 | C |
| ATOM | 131 | C | LEU | H | 18 | 8.906 | 49.377 | 15.775 | 1.00 | 23.34 | C |
| ATOM | 132 | O | LEU | H | 18 | 7.969 | 49.833 | 16.427 | 1.00 | 22.77 | O |
| ATOM | 133 | N | LYS | H | 19 | 8.904 | 49.261 | 14.455 | 1.00 | 23.26 | N |
| ATOM | 134 | CA | LYS | H | 19 | 7.730 | 49.531 | 13.647 | 1.00 | 23.52 | C |
| ATOM | 135 | CB | LYS | H | 19 | 7.740 | 50.991 | 13.186 | 1.00 | 23.86 | C |
| ATOM | 136 | CG | LYS | H | 19 | 6.600 | 51.373 | 12.262 | 1.00 | 26.10 | C |
| ATOM | 137 | CD | LYS | H | 19 | 6.453 | 52.880 | 12.184 | 1.00 | 29.50 | C |
| ATOM | 138 | CE | LYS | H | 19 | 5.314 | 53.271 | 11.245 | 1.00 | 31.98 | C |
| ATOM | 139 | NZ | LYS | H | 19 | 4.648 | 54.565 | 11.641 | 1.00 | 32.67 | N |
| ATOM | 140 | C | LYS | H | 19 | 7.717 | 48.566 | 12.457 | 1.00 | 23.05 | C |
| ATOM | 141 | O | LYS | H | 19 | 8.549 | 48.672 | 11.555 | 1.00 | 23.13 | O |
| ATOM | 142 | N | ILE | H | 20 | 6.784 | 47.621 | 12.471 | 1.00 | 22.44 | N |
| ATOM | 143 | CA | ILE | H | 20 | 6.669 | 46.621 | 11.394 | 1.00 | 21.90 | C |
| ATOM | 144 | CB | ILE | H | 20 | 6.715 | 45.154 | 11.940 | 1.00 | 21.95 | C |
| ATOM | 145 | CG1 | ILE | H | 20 | 5.627 | 44.924 | 12.982 | 1.00 | 21.57 | C |
| ATOM | 146 | CD1 | ILE | H | 20 | 5.883 | 43.772 | 13.923 | 1.00 | 22.52 | C |
| ATOM | 147 | CG2 | ILE | H | 20 | 8.091 | 44.854 | 12.509 | 1.00 | 22.14 | C |
| ATOM | 148 | C | ILE | H | 20 | 5.426 | 46.844 | 10.523 | 1.00 | 21.27 | C |
| ATOM | 149 | O | ILE | H | 20 | 4.383 | 47.260 | 11.028 | 1.00 | 21.13 | O |
| ATOM | 150 | N | SER | H | 21 | 5.552 | 46.569 | 9.225 | 1.00 | 20.49 | N |
| ATOM | 151 | CA | SER | H | 21 | 4.474 | 46.807 | 8.273 | 1.00 | 20.55 | C |
| ATOM | 152 | CB | SER | H | 21 | 4.988 | 47.590 | 7.071 | 1.00 | 20.66 | C |
| ATOM | 153 | OG | SER | H | 21 | 5.960 | 46.852 | 6.354 | 1.00 | 20.89 | O |
| ATOM | 154 | C | SER | H | 21 | 3.783 | 45.536 | 7.778 | 1.00 | 20.84 | C |
| ATOM | 155 | O | SER | H | 21 | 4.366 | 44.444 | 7.756 | 1.00 | 19.67 | O |
| ATOM | 156 | N | CYS | H | 22 | 2.530 | 45.712 | 7.378 | 1.00 | 21.34 | N |
| ATOM | 157 | CA | CYS | H | 22 | 1.705 | 44.640 | 6.872 | 1.00 | 22.24 | C |
| ATOM | 158 | CB | CYS | H | 22 | 0.758 | 44.122 | 7.958 | 1.00 | 21.80 | C |
| ATOM | 159 | SG | CYS | H | 22 | −0.570 | 43.029 | 7.396 | 1.00 | 22.48 | S |
| ATOM | 160 | C | CYS | H | 22 | 0.929 | 45.238 | 5.729 | 1.00 | 23.35 | C |
| ATOM | 161 | O | CYS | H | 22 | −0.007 | 46.004 | 5.942 | 1.00 | 23.09 | O |
| ATOM | 162 | N | GLN | H | 23 | 1.345 | 44.890 | 4.517 | 1.00 | 24.75 | N |
| ATOM | 163 | CA | GLN | H | 23 | 0.778 | 45.432 | 3.302 | 1.00 | 26.80 | C |
| ATOM | 164 | CB | GLN | H | 23 | 1.902 | 45.664 | 2.299 | 1.00 | 26.98 | C |
| ATOM | 165 | CG | GLN | H | 23 | 1.538 | 46.599 | 1.165 | 1.00 | 29.47 | C |
| ATOM | 166 | CD | GLN | H | 23 | 2.746 | 47.353 | 0.649 | 1.00 | 32.97 | C |
| ATOM | 167 | OE1 | GLN | H | 23 | 3.859 | 47.267 | 1.213 | 1.00 | 34.65 | O |
| ATOM | 168 | NE2 | GLN | H | 23 | 2.543 | 48.098 | −0.426 | 1.00 | 32.26 | N |
| ATOM | 169 | C | GLN | H | 23 | −0.283 | 44.519 | 2.679 | 1.00 | 27.72 | C |
| ATOM | 170 | O | GLN | H | 23 | −0.085 | 43.305 | 2.560 | 1.00 | 27.69 | O |
| ATOM | 171 | N | ALA | H | 24 | −1.385 | 45.122 | 2.245 | 1.00 | 29.08 | N |
| ATOM | 172 | CA | ALA | H | 24 | −2.523 | 44.383 | 1.708 | 1.00 | 30.89 | C |
| ATOM | 173 | CB | ALA | H | 24 | −3.815 | 44.901 | 2.308 | 1.00 | 30.63 | C |
| ATOM | 174 | C | ALA | H | 24 | −2.596 | 44.451 | 0.199 | 1.00 | 32.23 | C |
| ATOM | 175 | O | ALA | H | 24 | −2.337 | 45.487 | −0.406 | 1.00 | 32.97 | O |
| ATOM | 176 | N | PHE | H | 25 | −2.956 | 43.338 | −0.416 | 1.00 | 34.13 | N |
| ATOM | 177 | CA | PHE | H | 25 | −3.187 | 43.325 | −1.853 | 1.00 | 35.61 | C |
| ATOM | 178 | CB | PHE | H | 25 | −2.157 | 42.460 | −2.551 | 1.00 | 35.51 | C |
| ATOM | 179 | CG | PHE | H | 25 | −0.776 | 42.990 | −2.430 | 1.00 | 34.95 | C |
| ATOM | 180 | CD1 | PHE | H | 25 | −0.334 | 43.987 | −3.285 | 1.00 | 34.14 | C |
| ATOM | 181 | CE1 | PHE | H | 25 | 0.931 | 44.488 | −3.167 | 1.00 | 33.94 | C |
| ATOM | 182 | CZ | PHE | H | 25 | 1.767 | 44.006 | −2.182 | 1.00 | 33.22 | C |
| ATOM | 183 | CE2 | PHE | H | 25 | 1.339 | 43.022 | −1.318 | 1.00 | 32.51 | C |
| ATOM | 184 | CD2 | PHE | H | 25 | 0.073 | 42.520 | −1.441 | 1.00 | 33.92 | C |
| ATOM | 185 | C | PHE | H | 25 | −4.590 | 42.842 | −2.127 | 1.00 | 36.99 | C |
| ATOM | 186 | O | PHE | H | 25 | −4.866 | 41.628 | −2.120 | 1.00 | 37.82 | O |
| ATOM | 187 | N | GLY | H | 26 | −5.480 | 43.807 | −2.336 | 1.00 | 37.81 | N |
| ATOM | 188 | CA | GLY | H | 26 | −6.890 | 43.516 | −2.489 | 1.00 | 38.41 | C |
| ATOM | 189 | C | GLY | H | 26 | −7.661 | 44.775 | −2.788 | 1.00 | 39.16 | C |
| ATOM | 190 | O | GLY | H | 26 | −7.084 | 45.855 | −3.013 | 1.00 | 39.63 | O |
| ATOM | 191 | N | TYR | H | 27 | −8.976 | 44.619 | −2.811 | 1.00 | 39.20 | N |
| ATOM | 192 | CA | TYR | H | 27 | −9.890 | 45.721 | −3.013 | 1.00 | 39.18 | C |
| ATOM | 193 | CB | TYR | H | 27 | −10.935 | 45.352 | −4.090 | 1.00 | 40.24 | C |
| ATOM | 194 | CG | TYR | H | 27 | −12.187 | 46.228 | −4.104 | 1.00 | 43.61 | C |
| ATOM | 195 | CD1 | TYR | H | 27 | −12.205 | 47.448 | −4.793 | 1.00 | 46.87 | C |
| ATOM | 196 | CE1 | TYR | H | 27 | −13.356 | 48.264 | −4.802 | 1.00 | 49.67 | C |
| ATOM | 197 | CZ | TYR | H | 27 | −14.507 | 47.849 | −4.118 | 1.00 | 50.72 | C |
| ATOM | 198 | OH | TYR | H | 27 | −15.638 | 48.645 | −4.133 | 1.00 | 52.43 | O |
| ATOM | 199 | CE2 | TYR | H | 27 | −14.518 | 46.636 | −3.425 | 1.00 | 49.34 | C |
| ATOM | 200 | CD2 | TYR | H | 27 | −13.357 | 45.832 | −3.423 | 1.00 | 47.29 | C |
| ATOM | 201 | C | TYR | H | 27 | −10.555 | 45.978 | −1.677 | 1.00 | 37.70 | C |
| ATOM | 202 | O | TYR | H | 27 | −10.944 | 45.033 | −0.980 | 1.00 | 38.21 | O |
| ATOM | 203 | N | GLY | H | 28 | −10.686 | 47.248 | −1.316 | 1.00 | 35.84 | N |
| ATOM | 204 | CA | GLY | H | 28 | −11.460 | 47.614 | −0.132 | 1.00 | 33.46 | C |
| ATOM | 205 | C | GLY | H | 28 | −10.732 | 47.423 | 1.189 | 1.00 | 31.71 | C |
| ATOM | 206 | O | GLY | H | 28 | −11.314 | 46.924 | 2.156 | 1.00 | 31.58 | O |
| ATOM | 207 | N | PHE | H | 29 | −9.465 | 47.836 | 1.216 | 1.00 | 29.56 | N |
| ATOM | 208 | CA | PHE | H | 29 | −8.677 | 47.960 | 2.437 | 1.00 | 27.67 | C |
| ATOM | 209 | CB | PHE | H | 29 | −7.410 | 48.756 | 2.126 | 1.00 | 27.02 | C |
| ATOM | 210 | CG | PHE | H | 29 | −6.454 | 48.893 | 3.275 | 1.00 | 24.80 | C |
| ATOM | 211 | CD1 | PHE | H | 29 | −6.219 | 50.134 | 3.846 | 1.00 | 22.40 | C |
| ATOM | 212 | CE1 | PHE | H | 29 | −5.318 | 50.276 | 4.881 | 1.00 | 21.08 | C |
| ATOM | 213 | CZ | PHE | H | 29 | −4.628 | 49.167 | 5.366 | 1.00 | 21.00 | C |
| ATOM | 214 | CE2 | PHE | H | 29 | −4.840 | 47.925 | 4.807 | 1.00 | 20.75 | C |
| ATOM | 215 | CD2 | PHE | H | 29 | −5.748 | 47.792 | 3.760 | 1.00 | 23.78 | C |
| ATOM | 216 | C | PHE | H | 29 | −9.437 | 48.623 | 3.588 | 1.00 | 27.13 | C |
| ATOM | 217 | O | PHE | H | 29 | −9.302 | 48.210 | 4.732 | 1.00 | 27.21 | O |
| ATOM | 218 | N | ILE | H | 30 | −10.250 | 49.627 | 3.289 | 1.00 | 26.23 | N |
| ATOM | 219 | CA | ILE | H | 30 | −10.831 | 50.450 | 4.341 | 1.00 | 25.80 | C |
| ATOM | 220 | CB | ILE | H | 30 | −11.368 | 51.828 | 3.831 | 1.00 | 25.67 | C |
| ATOM | 221 | CG1 | ILE | H | 30 | −12.499 | 51.666 | 2.823 | 1.00 | 26.12 | C |
| ATOM | 222 | CD1 | ILE | H | 30 | −13.247 | 52.991 | 2.538 | 1.00 | 27.67 | C |
| ATOM | 223 | CG2 | ILE | H | 30 | −10.236 | 52.656 | 3.239 | 1.00 | 26.20 | C |
| ATOM | 224 | C | ILE | H | 30 | −11.864 | 49.709 | 5.186 | 1.00 | 25.54 | C |
| ATOM | 225 | O | ILE | H | 30 | −12.100 | 50.061 | 6.353 | 1.00 | 25.13 | O |
| ATOM | 226 | N | ASN | H | 31 | −12.444 | 48.667 | 4.596 | 1.00 | 24.74 | N |
| ATOM | 227 | CA | ASN | H | 31 | −13.330 | 47.776 | 5.320 | 1.00 | 24.28 | C |
| ATOM | 228 | CB | ASN | H | 31 | −14.533 | 47.397 | 4.441 | 1.00 | 24.74 | C |
| ATOM | 229 | CG | ASN | H | 31 | −15.420 | 48.595 | 4.104 | 1.00 | 25.99 | C |
| ATOM | 230 | OD1 | ASN | H | 31 | −15.847 | 49.359 | 4.985 | 1.00 | 28.39 | O |
| ATOM | 231 | ND2 | ASN | H | 31 | −15.708 | 48.757 | 2.823 | 1.00 | 26.31 | N |
| ATOM | 232 | C | ASN | H | 31 | −12.637 | 46.513 | 5.881 | 1.00 | 23.29 | C |
| ATOM | 233 | O | ASN | H | 31 | −13.308 | 45.590 | 6.343 | 1.00 | 23.18 | O |
| ATOM | 234 | N | TYR | H | 32 | −11.309 | 46.465 | 5.839 | 1.00 | 21.93 | N |
| ATOM | 235 | CA | TYR | H | 32 | −10.590 | 45.370 | 6.503 | 1.00 | 21.33 | C |
| ATOM | 236 | CB | TYR | H | 32 | −9.116 | 45.289 | 6.083 | 1.00 | 21.04 | C |
| ATOM | 237 | CG | TYR | H | 32 | −8.849 | 44.738 | 4.701 | 1.00 | 20.48 | C |
| ATOM | 238 | CD1 | TYR | H | 32 | −9.875 | 44.559 | 3.783 | 1.00 | 20.34 | C |
| ATOM | 239 | CE1 | TYR | H | 32 | −9.630 | 44.071 | 2.503 | 1.00 | 22.40 | C |
| ATOM | 240 | CZ | TYR | H | 32 | −8.324 | 43.767 | 2.117 | 1.00 | 23.85 | C |
| ATOM | 241 | OH | TYR | H | 32 | −8.079 | 43.295 | 0.850 | 1.00 | 26.22 | O |
| ATOM | 242 | CE2 | TYR | H | 32 | −7.276 | 43.942 | 3.005 | 1.00 | 23.63 | C |
| ATOM | 243 | CD2 | TYR | H | 32 | −7.548 | 44.439 | 4.300 | 1.00 | 22.47 | C |
| ATOM | 244 | C | TYR | H | 32 | −10.655 | 45.488 | 8.020 | 1.00 | 20.48 | C |
| ATOM | 245 | O | TYR | H | 32 | −10.791 | 46.584 | 8.578 | 1.00 | 20.69 | O |
| ATOM | 246 | N | LEU | H | 33 | −10.572 | 44.337 | 8.668 | 1.00 | 19.22 | N |
| ATOM | 247 | CA | LEU | H | 33 | −10.322 | 44.259 | 10.092 | 1.00 | 18.22 | C |
| ATOM | 248 | CB | LEU | H | 33 | −11.465 | 43.528 | 10.798 | 1.00 | 18.09 | C |
| ATOM | 249 | CG | LEU | H | 33 | −12.857 | 44.161 | 10.673 | 1.00 | 15.89 | C |
| ATOM | 250 | CD1 | LEU | H | 33 | −13.838 | 43.305 | 11.431 | 1.00 | 12.53 | C |
| ATOM | 251 | CD2 | LEU | H | 33 | −12.855 | 45.607 | 11.189 | 1.00 | 12.79 | C |
| ATOM | 252 | C | LEU | H | 33 | −9.026 | 43.494 | 10.234 | 1.00 | 18.05 | C |
| ATOM | 253 | O | LEU | H | 33 | −8.979 | 42.276 | 10.010 | 1.00 | 18.40 | O |
| ATOM | 254 | N | ILE | H | 34 | −7.968 | 44.218 | 10.569 | 1.00 | 17.23 | N |
| ATOM | 255 | CA | ILE | H | 34 | −6.639 | 43.651 | 10.587 | 1.00 | 16.74 | C |
| ATOM | 256 | CB | ILE | H | 34 | −5.613 | 44.595 | 9.935 | 1.00 | 16.89 | C |
| ATOM | 257 | CG1 | ILE | H | 34 | −5.937 | 44.840 | 8.452 | 1.00 | 17.34 | C |
| ATOM | 258 | CD1 | ILE | H | 34 | −5.509 | 43.723 | 7.515 | 1.00 | 18.24 | C |
| ATOM | 259 | CG2 | ILE | H | 34 | −4.206 | 44.052 | 10.103 | 1.00 | 16.78 | C |
| ATOM | 260 | C | ILE | H | 34 | −6.236 | 43.389 | 12.017 | 1.00 | 16.66 | C |
| ATOM | 261 | O | ILE | H | 34 | −6.307 | 44.284 | 12.884 | 1.00 | 16.42 | O |
| ATOM | 262 | N | GLU | H | 35 | −5.803 | 42.160 | 12.265 | 1.00 | 16.48 | N |
| ATOM | 263 | CA | GLU | H | 35 | −5.359 | 41.778 | 13.591 | 1.00 | 16.30 | C |
| ATOM | 264 | CB | GLU | H | 35 | −6.057 | 40.517 | 14.051 | 1.00 | 16.04 | C |
| ATOM | 265 | CG | GLU | H | 35 | −7.551 | 40.622 | 14.265 | 1.00 | 16.11 | C |
| ATOM | 266 | CD | GLU | H | 35 | −8.175 | 39.246 | 14.460 | 1.00 | 15.27 | C |
| ATOM | 267 | OE1 | GLU | H | 35 | −8.712 | 38.703 | 13.464 | 1.00 | 13.47 | O |
| ATOM | 268 | OE2 | GLU | H | 35 | −8.092 | 38.704 | 15.595 | 1.00 | 14.20 | O |
| ATOM | 269 | C | GLU | H | 35 | −3.863 | 41.538 | 13.626 | 1.00 | 16.76 | C |
| ATOM | 270 | O | GLU | H | 35 | −3.246 | 41.142 | 12.629 | 1.00 | 16.91 | O |
| ATOM | 271 | N | TRP | H | 36 | −3.298 | 41.761 | 14.801 | 1.00 | 17.00 | N |
| ATOM | 272 | CA | TRP | H | 36 | −1.921 | 41.444 | 15.072 | 1.00 | 17.41 | C |
| ATOM | 273 | CB | TRP | H | 36 | −1.171 | 42.708 | 15.448 | 1.00 | 17.35 | C |
| ATOM | 274 | CG | TRP | H | 36 | −1.031 | 43.629 | 14.309 | 1.00 | 16.52 | C |
| ATOM | 275 | CD1 | TRP | H | 36 | −1.924 | 44.568 | 13.913 | 1.00 | 16.21 | C |
| ATOM | 276 | NE1 | TRP | H | 36 | −1.442 | 45.249 | 12.823 | 1.00 | 17.31 | N |
| ATOM | 277 | CE2 | TRP | H | 36 | −0.222 | 44.734 | 12.483 | 1.00 | 16.80 | C |
| ATOM | 278 | CD2 | TRP | H | 36 | 0.068 | 43.703 | 13.399 | 1.00 | 16.05 | C |
| ATOM | 279 | CE3 | TRP | H | 36 | 1.277 | 43.012 | 13.279 | 1.00 | 17.09 | C |
| ATOM | 280 | CZ3 | TRP | H | 36 | 2.157 | 43.368 | 12.253 | 1.00 | 18.13 | C |
| ATOM | 281 | CH2 | TRP | H | 36 | 1.837 | 44.402 | 11.351 | 1.00 | 17.34 | C |
| ATOM | 282 | CZ2 | TRP | H | 36 | 0.650 | 45.089 | 11.447 | 1.00 | 17.94 | C |
| ATOM | 283 | C | TRP | H | 36 | −1.851 | 40.442 | 16.207 | 1.00 | 17.98 | C |
| ATOM | 284 | O | TRP | H | 36 | −2.534 | 40.590 | 17.225 | 1.00 | 17.72 | O |
| ATOM | 285 | N | ILE | H | 37 | −1.030 | 39.415 | 16.003 | 1.00 | 18.73 | N |
| ATOM | 286 | CA | ILE | H | 37 | −0.906 | 38.295 | 16.918 | 1.00 | 19.18 | C |
| ATOM | 287 | CB | ILE | H | 37 | −1.744 | 37.102 | 16.431 | 1.00 | 19.47 | C |
| ATOM | 288 | CG1 | ILE | H | 37 | −3.241 | 37.438 | 16.474 | 1.00 | 20.07 | C |
| ATOM | 289 | CD1 | ILE | H | 37 | −4.103 | 36.517 | 15.645 | 1.00 | 20.67 | C |
| ATOM | 290 | CG2 | ILE | H | 37 | −1.437 | 35.834 | 17.264 | 1.00 | 19.79 | C |
| ATOM | 291 | C | ILE | H | 37 | 0.554 | 37.876 | 16.958 | 1.00 | 19.78 | C |
| ATOM | 292 | O | ILE | H | 37 | 1.199 | 37.724 | 15.904 | 1.00 | 19.28 | O |
| ATOM | 293 | N | ARG | H | 38 | 1.087 | 37.677 | 18.161 | 1.00 | 20.29 | N |
| ATOM | 294 | CA | ARG | H | 38 | 2.469 | 37.225 | 18.259 | 1.00 | 21.29 | C |
| ATOM | 295 | CB | ARG | H | 38 | 3.341 | 38.262 | 18.973 | 1.00 | 20.88 | C |
| ATOM | 296 | CG | ARG | H | 38 | 3.239 | 38.220 | 20.471 | 1.00 | 21.25 | C |
| ATOM | 297 | CD | ARG | H | 38 | 4.084 | 39.282 | 21.129 | 1.00 | 21.49 | C |
| ATOM | 298 | NE | ARG | H | 38 | 4.042 | 39.114 | 22.572 | 1.00 | 20.73 | N |
| ATOM | 299 | CZ | ARG | H | 38 | 4.717 | 39.852 | 23.439 | 1.00 | 21.08 | C |
| ATOM | 300 | NH1 | ARG | H | 38 | 5.503 | 40.839 | 23.027 | 1.00 | 21.53 | N |
| ATOM | 301 | NH2 | ARG | H | 38 | 4.604 | 39.592 | 24.727 | 1.00 | 21.61 | N |
| ATOM | 302 | C | ARG | H | 38 | 2.629 | 35.831 | 18.876 | 1.00 | 22.34 | C |
| ATOM | 303 | O | ARG | H | 38 | 1.755 | 35.343 | 19.623 | 1.00 | 22.05 | O |
| ATOM | 304 | N | GLN | H | 39 | 3.763 | 35.208 | 18.559 | 1.00 | 23.77 | N |
| ATOM | 305 | CA | GLN | H | 39 | 4.075 | 33.871 | 19.037 | 1.00 | 25.44 | C |
| ATOM | 306 | CB | GLN | H | 39 | 3.675 | 32.841 | 17.989 | 1.00 | 24.76 | C |
| ATOM | 307 | CG | GLN | H | 39 | 3.920 | 31.425 | 18.400 | 1.00 | 23.74 | C |
| ATOM | 308 | CD | GLN | H | 39 | 3.274 | 30.442 | 17.460 | 1.00 | 23.31 | C |
| ATOM | 309 | OE1 | GLN | H | 39 | 3.400 | 30.562 | 16.237 | 1.00 | 21.85 | O |
| ATOM | 310 | NE2 | GLN | H | 39 | 2.565 | 29.462 | 18.024 | 1.00 | 20.93 | N |
| ATOM | 311 | C | GLN | H | 39 | 5.540 | 33.694 | 19.398 | 1.00 | 27.13 | C |
| ATOM | 312 | O | GLN | H | 39 | 6.414 | 33.719 | 18.527 | 1.00 | 27.30 | O |
| ATOM | 313 | N | MET | H | 40 | 5.794 | 33.485 | 20.686 | 1.00 | 29.72 | N |
| ATOM | 314 | CA | MET | H | 40 | 7.123 | 33.112 | 21.159 | 1.00 | 32.40 | C |
| ATOM | 315 | CB | MET | H | 40 | 7.256 | 33.271 | 22.672 | 1.00 | 33.11 | C |
| ATOM | 316 | CG | MET | H | 40 | 7.526 | 34.691 | 23.136 | 1.00 | 36.72 | C |
| ATOM | 317 | SD | MET | H | 40 | 6.135 | 35.515 | 23.942 | 1.00 | 44.92 | S |
| ATOM | 318 | CE | MET | H | 40 | 4.975 | 35.758 | 22.592 | 1.00 | 42.05 | C |
| ATOM | 319 | C | MET | H | 40 | 7.368 | 31.672 | 20.783 | 1.00 | 33.28 | C |
| ATOM | 320 | O | MET | H | 40 | 6.429 | 30.874 | 20.775 | 1.00 | 33.15 | O |
| ATOM | 321 | N | PRO | H | 41 | 8.636 | 31.327 | 20.486 | 1.00 | 34.60 | N |
| ATOM | 322 | CA | PRO | H | 41 | 8.943 | 29.993 | 19.928 | 1.00 | 35.17 | C |
| ATOM | 323 | CB | PRO | H | 41 | 10.452 | 30.057 | 19.631 | 1.00 | 35.27 | C |
| ATOM | 324 | CG | PRO | H | 41 | 10.852 | 31.535 | 19.812 | 1.00 | 35.68 | C |
| ATOM | 325 | CD | PRO | H | 41 | 9.856 | 32.099 | 20.792 | 1.00 | 34.78 | C |
| ATOM | 326 | C | PRO | H | 41 | 8.612 | 28.879 | 20.926 | 1.00 | 35.36 | C |
| ATOM | 327 | O | PRO | H | 41 | 8.981 | 28.976 | 22.096 | 1.00 | 35.61 | O |
| ATOM | 328 | N | GLY | H | 42 | 7.881 | 27.863 | 20.465 | 1.00 | 35.42 | N |
| ATOM | 329 | CA | GLY | H | 42 | 7.377 | 26.792 | 21.333 | 1.00 | 35.12 | C |
| ATOM | 330 | C | GLY | H | 42 | 6.114 | 27.126 | 22.124 | 1.00 | 34.76 | C |
| ATOM | 331 | O | GLY | H | 42 | 5.493 | 26.233 | 22.723 | 1.00 | 34.69 | O |
| ATOM | 332 | N | GLN | H | 43 | 5.727 | 28.403 | 22.102 | 1.00 | 34.08 | N |
| ATOM | 333 | CA | GLN | H | 43 | 4.621 | 28.924 | 22.919 | 1.00 | 33.34 | C |
| ATOM | 334 | CB | GLN | H | 43 | 5.024 | 30.254 | 23.581 | 1.00 | 33.96 | C |
| ATOM | 335 | CG | GLN | H | 43 | 6.293 | 30.192 | 24.430 | 1.00 | 36.96 | C |
| ATOM | 336 | CD | GLN | H | 43 | 6.196 | 29.148 | 25.523 | 1.00 | 41.58 | C |
| ATOM | 337 | OE1 | GLN | H | 43 | 5.270 | 29.181 | 26.352 | 1.00 | 43.16 | O |
| ATOM | 338 | NE2 | GLN | H | 43 | 7.142 | 28.203 | 25.529 | 1.00 | 42.18 | N |
| ATOM | 339 | C | GLN | H | 43 | 3.310 | 29.120 | 22.153 | 1.00 | 31.60 | C |
| ATOM | 340 | O | GLN | H | 43 | 3.197 | 28.780 | 20.972 | 1.00 | 31.06 | O |
| ATOM | 341 | N | GLY | H | 44 | 2.335 | 29.695 | 22.851 | 1.00 | 29.99 | N |
| ATOM | 342 | CA | GLY | H | 44 | 1.007 | 29.905 | 22.316 | 1.00 | 28.45 | C |
| ATOM | 343 | C | GLY | H | 44 | 0.864 | 31.243 | 21.622 | 1.00 | 27.65 | C |
| ATOM | 344 | O | GLY | H | 44 | 1.833 | 32.017 | 21.522 | 1.00 | 27.84 | O |
| ATOM | 345 | N | LEU | H | 45 | −0.350 | 31.489 | 21.133 | 1.00 | 26.12 | N |
| ATOM | 346 | CA | LEU | H | 45 | −0.701 | 32.692 | 20.405 | 1.00 | 25.00 | C |
| ATOM | 347 | CB | LEU | H | 45 | −1.812 | 32.377 | 19.400 | 1.00 | 24.85 | C |
| ATOM | 348 | CG | LEU | H | 45 | −1.493 | 31.352 | 18.308 | 1.00 | 24.42 | C |
| ATOM | 349 | CD1 | LEU | H | 45 | −2.756 | 30.930 | 17.567 | 1.00 | 24.13 | C |
| ATOM | 350 | CD2 | LEU | H | 45 | −0.452 | 31.893 | 17.332 | 1.00 | 22.20 | C |
| ATOM | 351 | C | LEU | H | 45 | −1.171 | 33.786 | 21.361 | 1.00 | 24.45 | C |
| ATOM | 352 | O | LEU | H | 45 | −1.968 | 33.530 | 22.263 | 1.00 | 24.56 | O |
| ATOM | 353 | N | GLU | H | 46 | −0.664 | 34.998 | 21.175 | 1.00 | 23.68 | N |
| ATOM | 354 | CA | GLU | H | 46 | −1.065 | 36.134 | 22.006 | 1.00 | 23.10 | C |
| ATOM | 355 | CB | GLU | H | 46 | 0.123 | 36.756 | 22.740 | 1.00 | 23.31 | C |
| ATOM | 356 | CG | GLU | H | 46 | 0.824 | 35.821 | 23.701 | 1.00 | 26.05 | C |
| ATOM | 357 | CD | GLU | H | 46 | 1.696 | 36.552 | 24.708 | 1.00 | 29.98 | C |
| ATOM | 358 | OE1 | GLU | H | 46 | 2.513 | 37.410 | 24.298 | 1.00 | 30.81 | O |
| ATOM | 359 | OE2 | GLU | H | 46 | 1.565 | 36.250 | 25.918 | 1.00 | 31.85 | O |
| ATOM | 360 | C | GLU | H | 46 | −1.662 | 37.166 | 21.104 | 1.00 | 22.06 | C |
| ATOM | 361 | O | GLU | H | 46 | −1.012 | 37.596 | 20.152 | 1.00 | 22.68 | O |
| ATOM | 362 | N | TRP | H | 47 | −2.898 | 37.551 | 21.403 | 1.00 | 20.78 | N |
| ATOM | 363 | CA | TRP | H | 47 | −3.634 | 38.529 | 20.621 | 1.00 | 19.39 | C |
| ATOM | 364 | CB | TRP | H | 47 | −5.126 | 38.283 | 20.790 | 1.00 | 18.98 | C |
| ATOM | 365 | CG | TRP | H | 47 | −6.002 | 39.050 | 19.874 | 1.00 | 18.56 | C |
| ATOM | 366 | CD1 | TRP | H | 47 | −6.382 | 38.697 | 18.604 | 1.00 | 18.84 | C |
| ATOM | 367 | NE1 | TRP | H | 47 | −7.216 | 39.655 | 18.072 | 1.00 | 18.22 | N |
| ATOM | 368 | CE2 | TRP | H | 47 | −7.397 | 40.643 | 19.002 | 1.00 | 18.90 | C |
| ATOM | 369 | CD2 | TRP | H | 47 | −6.651 | 40.286 | 20.158 | 1.00 | 18.60 | C |
| ATOM | 370 | CE3 | TRP | H | 47 | −6.661 | 41.143 | 21.265 | 1.00 | 17.96 | C |
| ATOM | 371 | CZ3 | TRP | H | 47 | −7.405 | 42.322 | 21.188 | 1.00 | 19.89 | C |
| ATOM | 372 | CH2 | TRP | H | 47 | −8.153 | 42.641 | 20.031 | 1.00 | 20.31 | C |
| ATOM | 373 | CZ2 | TRP | H | 47 | −8.156 | 41.819 | 18.930 | 1.00 | 19.38 | C |
| ATOM | 374 | C | TRP | H | 47 | −3.262 | 39.934 | 21.073 | 1.00 | 18.94 | C |
| ATOM | 375 | O | TRP | H | 47 | −3.382 | 40.265 | 22.252 | 1.00 | 18.69 | O |
| ATOM | 376 | N | ILE | H | 48 | −2.808 | 40.756 | 20.126 | 1.00 | 18.34 | N |
| ATOM | 377 | CA | ILE | H | 48 | −2.337 | 42.117 | 20.414 | 1.00 | 17.24 | C |
| ATOM | 378 | CB | ILE | H | 48 | −1.080 | 42.482 | 19.562 | 1.00 | 16.72 | C |
| ATOM | 379 | CG1 | ILE | H | 48 | 0.067 | 41.525 | 19.889 | 1.00 | 16.56 | C |
| ATOM | 380 | CD1 | ILE | H | 48 | 1.195 | 41.484 | 18.867 | 1.00 | 15.76 | C |
| ATOM | 381 | CG2 | ILE | H | 48 | −0.665 | 43.925 | 19.790 | 1.00 | 14.96 | C |
| ATOM | 382 | C | ILE | H | 48 | −3.439 | 43.146 | 20.198 | 1.00 | 17.41 | C |
| ATOM | 383 | O | ILE | H | 48 | −3.743 | 43.952 | 21.095 | 1.00 | 17.75 | O |
| ATOM | 384 | N | GLY | H | 49 | −4.033 | 43.133 | 19.009 | 1.00 | 17.09 | N |
| ATOM | 385 | CA | GLY | H | 49 | −5.032 | 44.135 | 18.677 | 1.00 | 16.97 | C |
| ATOM | 386 | C | GLY | H | 49 | −5.723 | 43.944 | 17.344 | 1.00 | 16.93 | C |
| ATOM | 387 | O | GLY | H | 49 | −5.336 | 43.096 | 16.539 | 1.00 | 16.35 | O |
| ATOM | 388 | N | LEU | H | 50 | −6.771 | 44.736 | 17.138 | 1.00 | 17.15 | N |
| ATOM | 389 | CA | LEU | H | 50 | −7.511 | 44.751 | 15.885 | 1.00 | 17.54 | C |
| ATOM | 390 | CB | LEU | H | 50 | −8.889 | 44.098 | 16.064 | 1.00 | 17.11 | C |
| ATOM | 391 | CG | LEU | H | 50 | −9.860 | 44.036 | 14.872 | 1.00 | 16.48 | C |
| ATOM | 392 | CD1 | LEU | H | 50 | −10.772 | 42.834 | 14.965 | 1.00 | 13.08 | C |
| ATOM | 393 | CD2 | LEU | H | 50 | −10.676 | 45.312 | 14.773 | 1.00 | 16.80 | C |
| ATOM | 394 | C | LEU | H | 50 | −7.655 | 46.202 | 15.427 | 1.00 | 17.98 | C |
| ATOM | 395 | O | LEU | H | 50 | −7.858 | 47.089 | 16.250 | 1.00 | 17.87 | O |
| ATOM | 396 | N | ILE | H | 51 | −7.557 | 46.434 | 14.121 | 1.00 | 18.53 | N |
| ATOM | 397 | CA | ILE | H | 51 | −7.759 | 47.770 | 13.571 | 1.00 | 19.06 | C |
| ATOM | 398 | CB | ILE | H | 51 | −6.406 | 48.490 | 13.306 | 1.00 | 19.02 | C |
| ATOM | 399 | CG1 | ILE | H | 51 | −6.640 | 49.906 | 12.785 | 1.00 | 18.40 | C |
| ATOM | 400 | CD1 | ILE | H | 51 | −5.419 | 50.800 | 12.883 | 1.00 | 17.98 | C |
| ATOM | 401 | CG2 | ILE | H | 51 | −5.531 | 47.685 | 12.346 | 1.00 | 18.96 | C |
| ATOM | 402 | C | ILE | H | 51 | −8.647 | 47.774 | 12.330 | 1.00 | 19.46 | C |
| ATOM | 403 | O | ILE | H | 51 | −8.557 | 46.891 | 11.493 | 1.00 | 19.16 | O |
| ATOM | 404 | N | ASN | H | 52 | −9.511 | 48.782 | 12.243 | 1.00 | 20.84 | N |
| ATOM | 405 | CA | ASN | H | 52 | −10.356 | 49.037 | 11.077 | 1.00 | 21.59 | C |
| ATOM | 406 | CB | ASN | H | 52 | −11.822 | 49.212 | 11.503 | 1.00 | 21.81 | C |
| ATOM | 407 | CG | ASN | H | 52 | −12.741 | 49.579 | 10.329 | 1.00 | 23.03 | C |
| ATOM | 408 | OD1 | ASN | H | 52 | −12.633 | 49.013 | 9.237 | 1.00 | 22.97 | O |
| ATOM | 409 | ND2 | ASN | H | 52 | −13.656 | 50.517 | 10.561 | 1.00 | 23.84 | N |
| ATOM | 410 | C | ASN | H | 52 | −9.868 | 50.314 | 10.404 | 1.00 | 22.08 | C |
| ATOM | 411 | O | ASN | H | 52 | −10.217 | 51.417 | 10.835 | 1.00 | 22.38 | O |
| ATOM | 412 | N | PRO | H | 52A | −9.058 | 50.175 | 9.342 | 1.00 | 22.60 | N |
| ATOM | 413 | CA | PRO | H | 52A | −8.418 | 51.327 | 8.696 | 1.00 | 22.72 | C |
| ATOM | 414 | CB | PRO | H | 52A | −7.702 | 50.705 | 7.498 | 1.00 | 22.77 | C |
| ATOM | 415 | CG | PRO | H | 52A | −7.489 | 49.258 | 7.895 | 1.00 | 22.28 | C |
| ATOM | 416 | CD | PRO | H | 52A | −8.711 | 48.905 | 8.672 | 1.00 | 22.59 | C |
| ATOM | 417 | C | PRO | H | 52A | −9.388 | 52.420 | 8.231 | 1.00 | 23.03 | C |
| ATOM | 418 | O | PRO | H | 52A | −9.006 | 53.596 | 8.178 | 1.00 | 23.40 | O |
| ATOM | 419 | N | GLY | H | 53 | −10.623 | 52.042 | 7.911 | 1.00 | 23.14 | N |
| ATOM | 420 | CA | GLY | H | 53 | −11.624 | 53.001 | 7.447 | 1.00 | 23.20 | C |
| ATOM | 421 | C | GLY | H | 53 | −12.330 | 53.823 | 8.515 | 1.00 | 23.30 | C |
| ATOM | 422 | O | GLY | H | 53 | −13.202 | 54.624 | 8.195 | 1.00 | 23.56 | O |
| ATOM | 423 | N | SER | H | 54 | −11.973 | 53.620 | 9.783 | 1.00 | 23.32 | N |
| ATOM | 424 | CA | SER | H | 54 | −12.561 | 54.378 | 10.888 | 1.00 | 23.06 | C |
| ATOM | 425 | CB | SER | H | 54 | −13.710 | 53.592 | 11.511 | 1.00 | 22.92 | C |
| ATOM | 426 | OG | SER | H | 54 | −13.217 | 52.477 | 12.245 | 1.00 | 22.27 | O |
| ATOM | 427 | C | SER | H | 54 | −11.491 | 54.634 | 11.929 | 1.00 | 23.41 | C |
| ATOM | 428 | O | SER | H | 54 | −10.335 | 54.247 | 11.735 | 1.00 | 23.81 | O |
| ATOM | 429 | N | ASP | H | 55 | −11.859 | 55.256 | 13.045 | 1.00 | 23.69 | N |
| ATOM | 430 | CA | ASP | H | 55 | −10.902 | 55.406 | 14.152 | 1.00 | 24.57 | C |
| ATOM | 431 | CB | ASP | H | 55 | −11.349 | 56.493 | 15.142 | 1.00 | 24.74 | C |
| ATOM | 432 | CG | ASP | H | 55 | −11.224 | 57.903 | 14.580 | 1.00 | 25.87 | C |
| ATOM | 433 | OD1 | ASP | H | 55 | −12.002 | 58.765 | 15.056 | 1.00 | 28.06 | O |
| ATOM | 434 | OD2 | ASP | H | 55 | −10.372 | 58.151 | 13.686 | 1.00 | 24.17 | O |
| ATOM | 435 | C | ASP | H | 55 | −10.672 | 54.119 | 14.945 | 1.00 | 24.45 | C |
| ATOM | 436 | O | ASP | H | 55 | −9.809 | 54.094 | 15.818 | 1.00 | 25.04 | O |
| ATOM | 437 | N | TYR | H | 56 | −11.439 | 53.066 | 14.651 | 1.00 | 24.18 | N |
| ATOM | 438 | CA | TYR | H | 56 | −11.526 | 51.903 | 15.536 | 1.00 | 23.31 | C |
| ATOM | 439 | CB | TYR | H | 56 | −12.714 | 51.005 | 15.192 | 1.00 | 23.83 | C |
| ATOM | 440 | CG | TYR | H | 56 | −13.024 | 50.022 | 16.300 | 1.00 | 25.55 | C |
| ATOM | 441 | CD1 | TYR | H | 56 | −13.801 | 50.408 | 17.408 | 1.00 | 27.47 | C |
| ATOM | 442 | CE1 | TYR | H | 56 | −14.081 | 49.513 | 18.446 | 1.00 | 27.59 | C |
| ATOM | 443 | CZ | TYR | H | 56 | −13.568 | 48.227 | 18.389 | 1.00 | 27.51 | C |
| ATOM | 444 | OH | TYR | H | 56 | −13.827 | 47.340 | 19.415 | 1.00 | 27.99 | O |
| ATOM | 445 | CE2 | TYR | H | 56 | −12.783 | 47.833 | 17.312 | 1.00 | 26.98 | C |
| ATOM | 446 | CD2 | TYR | H | 56 | −12.514 | 48.729 | 16.276 | 1.00 | 25.44 | C |
| ATOM | 447 | C | TYR | H | 56 | −10.283 | 51.053 | 15.651 | 1.00 | 22.58 | C |
| ATOM | 448 | O | TYR | H | 56 | −9.789 | 50.476 | 14.681 | 1.00 | 22.38 | O |
| ATOM | 449 | N | THR | H | 57 | −9.812 | 50.977 | 16.883 | 1.00 | 22.37 | N |
| ATOM | 450 | CA | THR | H | 57 | −8.790 | 50.040 | 17.302 | 1.00 | 21.94 | C |
| ATOM | 451 | CB | THR | H | 57 | −7.480 | 50.745 | 17.640 | 1.00 | 21.85 | C |
| ATOM | 452 | OG1 | THR | H | 57 | −7.715 | 51.704 | 18.680 | 1.00 | 21.43 | O |
| ATOM | 453 | CG2 | THR | H | 57 | −6.891 | 51.438 | 16.398 | 1.00 | 21.43 | C |
| ATOM | 454 | C | THR | H | 57 | −9.286 | 49.378 | 18.567 | 1.00 | 22.06 | C |
| ATOM | 455 | O | THR | H | 57 | −9.976 | 49.992 | 19.374 | 1.00 | 22.30 | O |
| ATOM | 456 | N | ASN | H | 58 | −8.958 | 48.107 | 18.722 | 1.00 | 22.14 | N |
| ATOM | 457 | CA | ASN | H | 58 | −9.127 | 47.434 | 19.986 | 1.00 | 21.84 | C |
| ATOM | 458 | CB | ASN | H | 58 | −10.212 | 46.376 | 19.922 | 1.00 | 21.39 | C |
| ATOM | 459 | CG | ASN | H | 58 | −10.753 | 46.041 | 21.292 | 1.00 | 22.27 | C |
| ATOM | 460 | OD1 | ASN | H | 58 | −10.058 | 45.443 | 22.112 | 1.00 | 23.71 | O |
| ATOM | 461 | ND2 | ASN | H | 58 | −11.994 | 46.441 | 21.559 | 1.00 | 21.02 | N |
| ATOM | 462 | C | ASN | H | 58 | −7.808 | 46.810 | 20.372 | 1.00 | 21.92 | C |
| ATOM | 463 | O | ASN | H | 58 | −7.163 | 46.145 | 19.561 | 1.00 | 22.10 | O |
| ATOM | 464 | N | TYR | H | 59 | −7.411 | 47.041 | 21.612 | 1.00 | 21.92 | N |
| ATOM | 465 | CA | TYR | H | 59 | −6.112 | 46.615 | 22.104 | 1.00 | 21.90 | C |
| ATOM | 466 | CB | TYR | H | 59 | −5.358 | 47.813 | 22.679 | 1.00 | 21.75 | C |
| ATOM | 467 | CG | TYR | H | 59 | −4.779 | 48.766 | 21.657 | 1.00 | 20.09 | C |
| ATOM | 468 | CD1 | TYR | H | 59 | −5.430 | 49.959 | 21.320 | 1.00 | 19.42 | C |
| ATOM | 469 | CE1 | TYR | H | 59 | −4.882 | 50.846 | 20.373 | 1.00 | 17.76 | C |
| ATOM | 470 | CZ | TYR | H | 59 | −3.666 | 50.535 | 19.786 | 1.00 | 18.18 | C |
| ATOM | 471 | OH | TYR | H | 59 | −3.070 | 51.370 | 18.866 | 1.00 | 19.18 | O |
| ATOM | 472 | CE2 | TYR | H | 59 | −3.017 | 49.367 | 20.119 | 1.00 | 19.38 | C |
| ATOM | 473 | CD2 | TYR | H | 59 | −3.572 | 48.492 | 21.048 | 1.00 | 18.38 | C |
| ATOM | 474 | C | TYR | H | 59 | −6.251 | 45.571 | 23.195 | 1.00 | 22.32 | C |
| ATOM | 475 | O | TYR | H | 59 | −7.233 | 45.574 | 23.956 | 1.00 | 22.12 | O |
| ATOM | 476 | N | ASN | H | 60 | −5.272 | 44.672 | 23.256 | 1.00 | 22.67 | N |
| ATOM | 477 | CA | ASN | H | 60 | −5.050 | 43.876 | 24.454 | 1.00 | 23.41 | C |
| ATOM | 478 | CB | ASN | H | 60 | −4.039 | 42.743 | 24.199 | 1.00 | 22.83 | C |
| ATOM | 479 | CG | ASN | H | 60 | −3.940 | 41.753 | 25.367 | 1.00 | 22.32 | C |
| ATOM | 480 | OD1 | ASN | H | 60 | −4.189 | 42.097 | 26.535 | 1.00 | 23.59 | O |
| ATOM | 481 | ND2 | ASN | H | 60 | −3.557 | 40.523 | 25.055 | 1.00 | 18.28 | N |
| ATOM | 482 | C | ASN | H | 60 | −4.537 | 44.832 | 25.531 | 1.00 | 24.02 | C |
| ATOM | 483 | O | ASN | H | 60 | −3.492 | 45.450 | 25.367 | 1.00 | 23.58 | O |
| ATOM | 484 | N | GLU | H | 61 | −5.288 | 44.938 | 26.622 | 1.00 | 25.59 | N |
| ATOM | 485 | CA | GLU | H | 61 | −4.946 | 45.805 | 27.752 | 1.00 | 27.80 | C |
| ATOM | 486 | CB | GLU | H | 61 | −5.996 | 45.659 | 28.877 | 1.00 | 28.19 | C |
| ATOM | 487 | CG | GLU | H | 61 | −5.492 | 45.845 | 30.325 | 1.00 | 33.52 | C |
| ATOM | 488 | CD | GLU | H | 61 | −6.634 | 45.931 | 31.371 | 1.00 | 40.09 | C |
| ATOM | 489 | OE1 | GLU | H | 61 | −7.574 | 45.082 | 31.354 | 1.00 | 40.96 | O |
| ATOM | 490 | OE2 | GLU | H | 61 | −6.576 | 46.859 | 32.221 | 1.00 | 41.84 | O |
| ATOM | 491 | C | GLU | H | 61 | −3.500 | 45.586 | 28.238 | 1.00 | 28.05 | C |
| ATOM | 492 | O | GLU | H | 61 | −2.807 | 46.556 | 28.565 | 1.00 | 28.01 | O |
| ATOM | 493 | N | ASN | H | 62 | −3.037 | 44.329 | 28.232 | 1.00 | 28.32 | N |
| ATOM | 494 | CA | ASN | H | 62 | −1.646 | 44.026 | 28.606 | 1.00 | 28.95 | C |
| ATOM | 495 | CB | ASN | H | 62 | −1.387 | 42.508 | 28.662 | 1.00 | 28.85 | C |
| ATOM | 496 | CG | ASN | H | 62 | −2.295 | 41.795 | 29.653 | 1.00 | 30.29 | C |
| ATOM | 497 | OD1 | ASN | H | 62 | −2.570 | 42.304 | 30.740 | 1.00 | 30.92 | O |
| ATOM | 498 | ND2 | ASN | H | 62 | −2.788 | 40.616 | 29.267 | 1.00 | 31.78 | N |
| ATOM | 499 | C | ASN | H | 62 | −0.571 | 44.713 | 27.748 | 1.00 | 28.96 | C |
| ATOM | 500 | O | ASN | H | 62 | 0.590 | 44.774 | 28.147 | 1.00 | 28.53 | O |
| ATOM | 501 | N | PHE | H | 63 | −0.960 | 45.227 | 26.581 | 1.00 | 29.45 | N |
| ATOM | 502 | CA | PHE | H | 63 | 0.007 | 45.838 | 25.668 | 1.00 | 30.17 | C |
| ATOM | 503 | CB | PHE | H | 63 | 0.143 | 45.027 | 24.374 | 1.00 | 30.05 | C |
| ATOM | 504 | CG | PHE | H | 63 | 0.483 | 43.584 | 24.588 | 1.00 | 29.68 | C |
| ATOM | 505 | CD1 | PHE | H | 63 | −0.303 | 42.588 | 24.025 | 1.00 | 28.97 | C |
| ATOM | 506 | CE1 | PHE | H | 63 | 0.003 | 41.246 | 24.210 | 1.00 | 27.92 | C |
| ATOM | 507 | CZ | PHE | H | 63 | 1.101 | 40.890 | 24.965 | 1.00 | 29.30 | C |
| ATOM | 508 | CE2 | PHE | H | 63 | 1.901 | 41.871 | 25.539 | 1.00 | 29.90 | C |
| ATOM | 509 | CD2 | PHE | H | 63 | 1.589 | 43.215 | 25.349 | 1.00 | 30.34 | C |
| ATOM | 510 | C | PHE | H | 63 | −0.246 | 47.305 | 25.329 | 1.00 | 30.79 | C |
| ATOM | 511 | O | PHE | H | 63 | 0.597 | 47.934 | 24.679 | 1.00 | 30.96 | O |
| ATOM | 512 | N | LYS | H | 64 | −1.394 | 47.851 | 25.743 | 1.00 | 31.54 | N |
| ATOM | 513 | CA | LYS | H | 64 | −1.616 | 49.299 | 25.630 | 1.00 | 32.42 | C |
| ATOM | 514 | CB | LYS | H | 64 | −2.962 | 49.724 | 26.254 | 1.00 | 32.96 | C |
| ATOM | 515 | CG | LYS | H | 64 | −2.885 | 50.261 | 27.708 | 1.00 | 34.50 | C |
| ATOM | 516 | CD | LYS | H | 64 | −4.127 | 49.929 | 28.555 | 1.00 | 38.04 | C |
| ATOM | 517 | CE | LYS | H | 64 | −5.306 | 50.873 | 28.280 | 1.00 | 39.88 | C |
| ATOM | 518 | NZ | LYS | H | 64 | −6.557 | 50.412 | 28.957 | 1.00 | 40.12 | N |
| ATOM | 519 | C | LYS | H | 64 | −0.445 | 49.950 | 26.356 | 1.00 | 32.19 | C |
| ATOM | 520 | O | LYS | H | 64 | −0.067 | 49.525 | 27.454 | 1.00 | 32.71 | O |
| ATOM | 521 | N | GLY | H | 65 | 0.164 | 50.949 | 25.749 | 1.00 | 31.89 | N |
| ATOM | 522 | CA | GLY | H | 65 | 1.349 | 51.546 | 26.381 | 1.00 | 31.51 | C |
| ATOM | 523 | C | GLY | H | 65 | 2.634 | 51.192 | 25.655 | 1.00 | 30.78 | C |
| ATOM | 524 | O | GLY | H | 65 | 3.537 | 52.026 | 25.565 | 1.00 | 31.08 | O |
| ATOM | 525 | N | GLN | H | 66 | 2.707 | 49.964 | 25.130 | 1.00 | 29.64 | N |
| ATOM | 526 | CA | GLN | H | 66 | 3.781 | 49.558 | 24.220 | 1.00 | 28.53 | C |
| ATOM | 527 | CB | GLN | H | 66 | 4.321 | 48.183 | 24.596 | 1.00 | 28.73 | C |
| ATOM | 528 | CG | GLN | H | 66 | 5.068 | 48.130 | 25.923 | 1.00 | 31.18 | C |
| ATOM | 529 | CD | GLN | H | 66 | 5.414 | 46.698 | 26.333 | 1.00 | 34.22 | C |
| ATOM | 530 | OE1 | GLN | H | 66 | 6.394 | 46.104 | 25.845 | 1.00 | 34.14 | O |
| ATOM | 531 | NE2 | GLN | H | 66 | 4.604 | 46.135 | 27.233 | 1.00 | 34.58 | N |
| ATOM | 532 | C | GLN | H | 66 | 3.316 | 49.532 | 22.762 | 1.00 | 27.43 | C |
| ATOM | 533 | O | GLN | H | 66 | 4.042 | 49.962 | 21.866 | 1.00 | 27.03 | O |
| ATOM | 534 | N | ALA | H | 67 | 2.107 | 49.029 | 22.527 | 1.00 | 26.19 | N |
| ATOM | 535 | CA | ALA | H | 67 | 1.637 | 48.787 | 21.164 | 1.00 | 25.13 | C |
| ATOM | 536 | CB | ALA | H | 67 | 0.940 | 47.429 | 21.068 | 1.00 | 24.78 | C |
| ATOM | 537 | C | ALA | H | 67 | 0.754 | 49.906 | 20.594 | 1.00 | 24.48 | C |
| ATOM | 538 | O | ALA | H | 67 | −0.199 | 50.359 | 21.226 | 1.00 | 24.22 | O |
| ATOM | 539 | N | THR | H | 68 | 1.096 | 50.341 | 19.389 | 1.00 | 23.91 | N |
| ATOM | 540 | CA | THR | H | 68 | 0.295 | 51.293 | 18.637 | 1.00 | 23.67 | C |
| ATOM | 541 | CB | THR | H | 68 | 1.049 | 52.643 | 18.472 | 1.00 | 23.58 | C |
| ATOM | 542 | OG1 | THR | H | 68 | 1.421 | 53.126 | 19.768 | 1.00 | 24.32 | O |
| ATOM | 543 | CG2 | THR | H | 68 | 0.185 | 53.699 | 17.795 | 1.00 | 23.16 | C |
| ATOM | 544 | C | THR | H | 68 | −0.053 | 50.671 | 17.283 | 1.00 | 23.53 | C |
| ATOM | 545 | O | THR | H | 68 | 0.847 | 50.315 | 16.495 | 1.00 | 23.49 | O |
| ATOM | 546 | N | LEU | H | 69 | −1.349 | 50.501 | 17.030 | 1.00 | 23.28 | N |
| ATOM | 547 | CA | LEU | H | 69 | −1.808 | 50.059 | 15.710 | 1.00 | 23.35 | C |
| ATOM | 548 | CB | LEU | H | 69 | −3.033 | 49.132 | 15.783 | 1.00 | 22.90 | C |
| ATOM | 549 | CG | LEU | H | 69 | −3.042 | 47.805 | 16.579 | 1.00 | 22.56 | C |
| ATOM | 550 | CD1 | LEU | H | 69 | −3.802 | 46.726 | 15.859 | 1.00 | 21.50 | C |
| ATOM | 551 | CD2 | LEU | H | 69 | −1.690 | 47.270 | 16.856 | 1.00 | 22.72 | C |
| ATOM | 552 | C | LEU | H | 69 | −2.119 | 51.291 | 14.891 | 1.00 | 23.68 | C |
| ATOM | 553 | O | LEU | H | 69 | −2.562 | 52.297 | 15.438 | 1.00 | 24.01 | O |
| ATOM | 554 | N | SER | H | 70 | −1.845 | 51.230 | 13.590 | 1.00 | 24.09 | N |
| ATOM | 555 | CA | SER | H | 70 | −2.169 | 52.328 | 12.669 | 1.00 | 24.67 | C |
| ATOM | 556 | CB | SER | H | 70 | −1.178 | 53.501 | 12.791 | 1.00 | 24.26 | C |
| ATOM | 557 | OG | SER | H | 70 | 0.118 | 53.137 | 12.363 | 1.00 | 23.47 | O |
| ATOM | 558 | C | SER | H | 70 | −2.231 | 51.810 | 11.245 | 1.00 | 25.21 | C |
| ATOM | 559 | O | SER | H | 70 | −1.875 | 50.661 | 10.995 | 1.00 | 25.47 | O |
| ATOM | 560 | N | ALA | H | 71 | −2.688 | 52.653 | 10.322 | 1.00 | 26.03 | N |
| ATOM | 561 | CA | ALA | H | 71 | −2.833 | 52.256 | 8.924 | 1.00 | 27.00 | C |
| ATOM | 562 | CB | ALA | H | 71 | −4.091 | 51.417 | 8.739 | 1.00 | 27.19 | C |
| ATOM | 563 | C | ALA | H | 71 | −2.853 | 53.446 | 7.975 | 1.00 | 27.63 | C |
| ATOM | 564 | O | ALA | H | 71 | −3.499 | 54.453 | 8.247 | 1.00 | 27.78 | O |
| ATOM | 565 | N | ASP | H | 72 | −2.135 | 53.317 | 6.864 | 1.00 | 28.46 | N |
| ATOM | 566 | CA | ASP | H | 72 | −2.179 | 54.300 | 5.782 | 1.00 | 29.18 | C |
| ATOM | 567 | CB | ASP | H | 72 | −0.763 | 54.573 | 5.259 | 1.00 | 29.43 | C |
| ATOM | 568 | CG | ASP | H | 72 | −0.736 | 55.464 | 4.024 | 1.00 | 31.25 | C |
| ATOM | 569 | OD1 | ASP | H | 72 | −1.716 | 56.191 | 3.736 | 1.00 | 32.75 | O |
| ATOM | 570 | OD2 | ASP | H | 72 | 0.296 | 55.434 | 3.328 | 1.00 | 34.39 | O |
| ATOM | 571 | C | ASP | H | 72 | −3.108 | 53.807 | 4.668 | 1.00 | 29.31 | C |
| ATOM | 572 | O | ASP | H | 72 | −2.758 | 52.899 | 3.906 | 1.00 | 29.19 | O |
| ATOM | 573 | N | LYS | H | 73 | −4.289 | 54.418 | 4.585 | 1.00 | 29.65 | N |
| ATOM | 574 | CA | LYS | H | 73 | −5.270 | 54.106 | 3.536 | 1.00 | 30.16 | C |
| ATOM | 575 | CB | LYS | H | 73 | −6.570 | 54.879 | 3.766 | 1.00 | 30.29 | C |
| ATOM | 576 | CG | LYS | H | 73 | −7.123 | 54.758 | 5.173 | 1.00 | 31.89 | C |
| ATOM | 577 | CD | LYS | H | 73 | −8.530 | 55.361 | 5.269 | 1.00 | 34.86 | C |
| ATOM | 578 | CE | LYS | H | 73 | −8.514 | 56.890 | 5.407 | 1.00 | 35.32 | C |
| ATOM | 579 | NZ | LYS | H | 73 | −7.679 | 57.347 | 6.563 | 1.00 | 36.02 | N |
| ATOM | 580 | C | LYS | H | 73 | −4.781 | 54.370 | 2.102 | 1.00 | 29.97 | C |
| ATOM | 581 | O | LYS | H | 73 | −5.299 | 53.786 | 1.162 | 1.00 | 29.72 | O |
| ATOM | 582 | N | SER | H | 74 | −3.804 | 55.256 | 1.926 | 1.00 | 30.09 | N |
| ATOM | 583 | CA | SER | H | 74 | −3.308 | 55.527 | 0.576 | 1.00 | 30.38 | C |
| ATOM | 584 | CB | SER | H | 74 | −2.579 | 56.879 | 0.491 | 1.00 | 30.32 | C |
| ATOM | 585 | OG | SER | H | 74 | −1.300 | 56.819 | 1.094 | 1.00 | 32.65 | O |
| ATOM | 586 | C | SER | H | 74 | −2.465 | 54.367 | 0.005 | 1.00 | 29.98 | C |
| ATOM | 587 | O | SER | H | 74 | −2.448 | 54.144 | −1.216 | 1.00 | 30.55 | O |
| ATOM | 588 | N | SER | H | 75 | −1.798 | 53.613 | 0.878 | 1.00 | 28.93 | N |
| ATOM | 589 | CA | SER | H | 75 | −0.925 | 52.529 | 0.427 | 1.00 | 27.66 | C |
| ATOM | 590 | CB | SER | H | 75 | 0.523 | 52.762 | 0.888 | 1.00 | 28.03 | C |
| ATOM | 591 | OG | SER | H | 75 | 0.615 | 52.948 | 2.296 | 1.00 | 28.02 | O |
| ATOM | 592 | C | SER | H | 75 | −1.391 | 51.141 | 0.855 | 1.00 | 26.88 | C |
| ATOM | 593 | O | SER | H | 75 | −0.695 | 50.158 | 0.597 | 1.00 | 26.87 | O |
| ATOM | 594 | N | SER | H | 76 | −2.563 | 51.062 | 1.490 | 1.00 | 25.60 | N |
| ATOM | 595 | CA | SER | H | 76 | −3.109 | 49.794 | 1.993 | 1.00 | 24.61 | C |
| ATOM | 596 | CB | SER | H | 76 | −3.591 | 48.905 | 0.834 | 1.00 | 24.87 | C |
| ATOM | 597 | OG | SER | H | 76 | −4.724 | 49.468 | 0.200 | 1.00 | 24.92 | O |
| ATOM | 598 | C | SER | H | 76 | −2.113 | 49.036 | 2.882 | 1.00 | 23.87 | C |
| ATOM | 599 | O | SER | H | 76 | −1.946 | 47.822 | 2.760 | 1.00 | 23.46 | O |
| ATOM | 600 | N | THR | H | 77 | −1.441 | 49.769 | 3.762 | 1.00 | 23.20 | N |
| ATOM | 601 | CA | THR | H | 77 | −0.471 | 49.177 | 4.676 | 1.00 | 22.54 | C |
| ATOM | 602 | CB | THR | H | 77 | 0.931 | 49.820 | 4.536 | 1.00 | 22.39 | C |
| ATOM | 603 | OG1 | THR | H | 77 | 1.225 | 50.060 | 3.156 | 1.00 | 23.30 | O |
| ATOM | 604 | CG2 | THR | H | 77 | 2.000 | 48.922 | 5.123 | 1.00 | 21.29 | C |
| ATOM | 605 | C | THR | H | 77 | −0.940 | 49.403 | 6.093 | 1.00 | 22.01 | C |
| ATOM | 606 | O | THR | H | 77 | −1.328 | 50.515 | 6.444 | 1.00 | 22.62 | O |
| ATOM | 607 | N | ALA | H | 78 | −0.897 | 48.356 | 6.903 | 1.00 | 21.13 | N |
| ATOM | 608 | CA | ALA | H | 78 | −1.128 | 48.483 | 8.335 | 1.00 | 20.77 | C |
| ATOM | 609 | CB | ALA | H | 78 | −2.024 | 47.358 | 8.832 | 1.00 | 20.67 | C |
| ATOM | 610 | C | ALA | H | 78 | 0.210 | 48.446 | 9.052 | 1.00 | 20.62 | C |
| ATOM | 611 | O | ALA | H | 78 | 1.182 | 47.875 | 8.533 | 1.00 | 20.98 | O |
| ATOM | 612 | N | TYR | H | 79 | 0.259 | 49.038 | 10.240 | 1.00 | 20.01 | N |
| ATOM | 613 | CA | TYR | H | 79 | 1.498 | 49.124 | 11.001 | 1.00 | 20.02 | C |
| ATOM | 614 | CB | TYR | H | 79 | 2.008 | 50.563 | 10.989 | 1.00 | 20.31 | C |
| ATOM | 615 | CG | TYR | H | 79 | 2.394 | 51.082 | 9.626 | 1.00 | 21.07 | C |
| ATOM | 616 | CD1 | TYR | H | 79 | 3.647 | 50.779 | 9.070 | 1.00 | 22.01 | C |
| ATOM | 617 | CE1 | TYR | H | 79 | 4.012 | 51.266 | 7.809 | 1.00 | 21.75 | C |
| ATOM | 618 | CZ | TYR | H | 79 | 3.117 | 52.068 | 7.107 | 1.00 | 21.19 | C |
| ATOM | 619 | OH | TYR | H | 79 | 3.461 | 52.549 | 5.872 | 1.00 | 20.04 | O |
| ATOM | 620 | CE2 | TYR | H | 79 | 1.868 | 52.382 | 7.646 | 1.00 | 20.84 | C |
| ATOM | 621 | CD2 | TYR | H | 79 | 1.519 | 51.893 | 8.897 | 1.00 | 20.20 | C |
| ATOM | 622 | C | TYR | H | 79 | 1.349 | 48.675 | 12.450 | 1.00 | 19.92 | C |
| ATOM | 623 | O | TYR | H | 79 | 0.289 | 48.860 | 13.067 | 1.00 | 20.31 | O |
| ATOM | 624 | N | LEU | H | 80 | 2.422 | 48.102 | 12.993 | 1.00 | 19.68 | N |
| ATOM | 625 | CA | LEU | H | 80 | 2.526 | 47.843 | 14.425 | 1.00 | 19.84 | C |
| ATOM | 626 | CB | LEU | H | 80 | 2.535 | 46.336 | 14.727 | 1.00 | 19.22 | C |
| ATOM | 627 | CG | LEU | H | 80 | 2.626 | 45.823 | 16.180 | 1.00 | 18.83 | C |
| ATOM | 628 | CD1 | LEU | H | 80 | 1.544 | 46.383 | 17.115 | 1.00 | 16.90 | C |
| ATOM | 629 | CD2 | LEU | H | 80 | 2.577 | 44.325 | 16.226 | 1.00 | 18.10 | C |
| ATOM | 630 | C | LEU | H | 80 | 3.761 | 48.539 | 14.998 | 1.00 | 20.53 | C |
| ATOM | 631 | O | LEU | H | 80 | 4.880 | 48.274 | 14.577 | 1.00 | 20.77 | O |
| ATOM | 632 | N | GLN | H | 81 | 3.542 | 49.459 | 15.933 | 1.00 | 21.68 | N |
| ATOM | 633 | CA | GLN | H | 81 | 4.634 | 50.072 | 16.692 | 1.00 | 23.04 | C |
| ATOM | 634 | CB | GLN | H | 81 | 4.456 | 51.595 | 16.820 | 1.00 | 23.55 | C |
| ATOM | 635 | CG | GLN | H | 81 | 4.911 | 52.418 | 15.622 | 1.00 | 25.45 | C |
| ATOM | 636 | CD | GLN | H | 81 | 4.187 | 53.765 | 15.513 | 1.00 | 28.30 | C |
| ATOM | 637 | OE1 | GLN | H | 81 | 2.955 | 53.834 | 15.549 | 1.00 | 29.27 | O |
| ATOM | 638 | NE2 | GLN | H | 81 | 4.955 | 54.837 | 15.356 | 1.00 | 29.56 | N |
| ATOM | 639 | C | GLN | H | 81 | 4.662 | 49.464 | 18.080 | 1.00 | 23.16 | C |
| ATOM | 640 | O | GLN | H | 81 | 3.617 | 49.197 | 18.672 | 1.00 | 22.83 | O |
| ATOM | 641 | N | TRP | H | 82 | 5.869 | 49.276 | 18.597 | 1.00 | 23.89 | N |
| ATOM | 642 | CA | TRP | H | 82 | 6.091 | 48.717 | 19.930 | 1.00 | 24.75 | C |
| ATOM | 643 | CB | TRP | H | 82 | 6.463 | 47.233 | 19.834 | 1.00 | 24.51 | C |
| ATOM | 644 | CG | TRP | H | 82 | 6.214 | 46.464 | 21.076 | 1.00 | 24.78 | C |
| ATOM | 645 | CD1 | TRP | H | 82 | 6.963 | 46.477 | 22.213 | 1.00 | 25.67 | C |
| ATOM | 646 | NE1 | TRP | H | 82 | 6.420 | 45.623 | 23.152 | 1.00 | 25.54 | N |
| ATOM | 647 | CE2 | TRP | H | 82 | 5.302 | 45.038 | 22.620 | 1.00 | 25.13 | C |
| ATOM | 648 | CD2 | TRP | H | 82 | 5.134 | 45.551 | 21.316 | 1.00 | 24.34 | C |
| ATOM | 649 | CE3 | TRP | H | 82 | 4.051 | 45.109 | 20.551 | 1.00 | 23.07 | C |
| ATOM | 650 | CZ3 | TRP | H | 82 | 3.175 | 44.194 | 21.102 | 1.00 | 23.52 | C |
| ATOM | 651 | CH2 | TRP | H | 82 | 3.360 | 43.704 | 22.400 | 1.00 | 24.89 | C |
| ATOM | 652 | CZ2 | TRP | H | 82 | 4.417 | 44.114 | 23.178 | 1.00 | 25.46 | C |
| ATOM | 653 | C | TRP | H | 82 | 7.234 | 49.494 | 20.570 | 1.00 | 25.13 | C |
| ATOM | 654 | O | TRP | H | 82 | 8.337 | 49.510 | 20.022 | 1.00 | 25.24 | O |
| ATOM | 655 | N | SER | H | 82A | 6.970 | 50.133 | 21.711 | 1.00 | 25.43 | N |
| ATOM | 656 | CA | SER | H | 82A | 7.985 | 50.922 | 22.412 | 1.00 | 26.06 | C |
| ATOM | 657 | CB | SER | H | 82A | 7.397 | 52.264 | 22.869 | 1.00 | 26.36 | C |
| ATOM | 658 | OG | SER | H | 82A | 6.325 | 52.060 | 23.784 | 1.00 | 27.93 | O |
| ATOM | 659 | C | SER | H | 82A | 8.550 | 50.138 | 23.596 | 1.00 | 25.90 | C |
| ATOM | 660 | O | SER | H | 82A | 7.963 | 49.140 | 24.010 | 1.00 | 26.04 | O |
| ATOM | 661 | N | SER | H | 82B | 9.685 | 50.583 | 24.136 | 1.00 | 25.83 | N |
| ATOM | 662 | CA | SER | H | 82B | 10.307 | 49.926 | 25.293 | 1.00 | 25.97 | C |
| ATOM | 663 | CB | SER | H | 82B | 9.590 | 50.312 | 26.596 | 1.00 | 25.92 | C |
| ATOM | 664 | OG | SER | H | 82B | 9.500 | 51.713 | 26.734 | 1.00 | 26.79 | O |
| ATOM | 665 | C | SER | H | 82B | 10.333 | 48.397 | 25.177 | 1.00 | 25.77 | C |
| ATOM | 666 | O | SER | H | 82B | 9.853 | 47.700 | 26.063 | 1.00 | 26.27 | O |
| ATOM | 667 | N | LEU | H | 82C | 10.899 | 47.884 | 24.091 | 1.00 | 25.59 | N |
| ATOM | 668 | CA | LEU | H | 82C | 11.021 | 46.449 | 23.882 | 1.00 | 25.15 | C |
| ATOM | 669 | CB | LEU | H | 82C | 11.795 | 46.181 | 22.607 | 1.00 | 24.62 | C |
| ATOM | 670 | CG | LEU | H | 82C | 11.054 | 46.579 | 21.345 | 1.00 | 24.45 | C |
| ATOM | 671 | CD1 | LEU | H | 82C | 12.008 | 46.665 | 20.177 | 1.00 | 23.44 | C |
| ATOM | 672 | CD2 | LEU | H | 82C | 9.935 | 45.576 | 21.052 | 1.00 | 25.78 | C |
| ATOM | 673 | C | LEU | H | 82C | 11.716 | 45.763 | 25.053 | 1.00 | 25.26 | C |
| ATOM | 674 | O | LEU | H | 82C | 12.658 | 46.305 | 25.631 | 1.00 | 25.54 | O |
| ATOM | 675 | N | LYS | H | 83 | 11.234 | 44.579 | 25.410 | 1.00 | 25.08 | N |
| ATOM | 676 | CA | LYS | H | 83 | 11.902 | 43.753 | 26.415 | 1.00 | 25.30 | C |
| ATOM | 677 | CB | LYS | H | 83 | 10.976 | 43.470 | 27.592 | 1.00 | 25.86 | C |
| ATOM | 678 | CG | LYS | H | 83 | 10.770 | 44.669 | 28.511 | 1.00 | 27.61 | C |
| ATOM | 679 | CD | LYS | H | 83 | 9.358 | 44.717 | 29.055 | 1.00 | 31.45 | C |
| ATOM | 680 | CE | LYS | H | 83 | 8.918 | 43.371 | 29.603 | 1.00 | 34.10 | C |
| ATOM | 681 | NZ | LYS | H | 83 | 7.435 | 43.333 | 29.875 | 1.00 | 37.45 | N |
| ATOM | 682 | C | LYS | H | 83 | 12.314 | 42.462 | 25.760 | 1.00 | 24.72 | C |
| ATOM | 683 | O | LYS | H | 83 | 11.742 | 42.080 | 24.747 | 1.00 | 24.69 | O |
| ATOM | 684 | N | ALA | H | 84 | 13.317 | 41.798 | 26.326 | 1.00 | 24.37 | N |
| ATOM | 685 | CA | ALA | H | 84 | 13.832 | 40.552 | 25.758 | 1.00 | 23.65 | C |
| ATOM | 686 | CB | ALA | H | 84 | 14.925 | 39.980 | 26.647 | 1.00 | 23.98 | C |
| ATOM | 687 | C | ALA | H | 84 | 12.706 | 39.546 | 25.587 | 1.00 | 23.31 | C |
| ATOM | 688 | O | ALA | H | 84 | 12.697 | 38.765 | 24.637 | 1.00 | 23.21 | O |
| ATOM | 689 | N | SER | H | 85 | 11.751 | 39.594 | 26.510 | 1.00 | 22.85 | N |
| ATOM | 690 | CA | SER | H | 85 | 10.616 | 38.696 | 26.511 | 1.00 | 22.74 | C |
| ATOM | 691 | CB | SER | H | 85 | 9.951 | 38.673 | 27.901 | 1.00 | 23.09 | C |
| ATOM | 692 | OG | SER | H | 85 | 9.419 | 39.937 | 28.265 | 1.00 | 24.10 | O |
| ATOM | 693 | C | SER | H | 85 | 9.598 | 39.039 | 25.422 | 1.00 | 22.35 | C |
| ATOM | 694 | O | SER | H | 85 | 8.603 | 38.327 | 25.269 | 1.00 | 22.84 | O |
| ATOM | 695 | N | ASP | H | 86 | 9.845 | 40.125 | 24.677 | 1.00 | 21.33 | N |
| ATOM | 696 | CA | ASP | H | 86 | 9.039 | 40.489 | 23.502 | 1.00 | 19.77 | C |
| ATOM | 697 | CB | ASP | H | 86 | 9.004 | 42.009 | 23.311 | 1.00 | 19.75 | C |
| ATOM | 698 | CG | ASP | H | 86 | 8.153 | 42.715 | 24.352 | 1.00 | 20.05 | C |
| ATOM | 699 | OD1 | ASP | H | 86 | 8.552 | 43.815 | 24.791 | 1.00 | 20.00 | O |
| ATOM | 700 | OD2 | ASP | H | 86 | 7.082 | 42.181 | 24.730 | 1.00 | 20.43 | O |
| ATOM | 701 | C | ASP | H | 86 | 9.545 | 39.806 | 22.231 | 1.00 | 19.09 | C |
| ATOM | 702 | O | ASP | H | 86 | 8.989 | 39.990 | 21.141 | 1.00 | 18.71 | O |
| ATOM | 703 | N | THR | H | 87 | 10.621 | 39.038 | 22.366 | 1.00 | 18.53 | N |
| ATOM | 704 | CA | THR | H | 87 | 11.131 | 38.226 | 21.261 | 1.00 | 18.09 | C |
| ATOM | 705 | CB | THR | H | 87 | 12.361 | 37.430 | 21.715 | 1.00 | 18.22 | C |
| ATOM | 706 | OG1 | THR | H | 87 | 13.425 | 38.346 | 22.024 | 1.00 | 18.13 | O |
| ATOM | 707 | CG2 | THR | H | 87 | 12.806 | 36.420 | 20.656 | 1.00 | 16.97 | C |
| ATOM | 708 | C | THR | H | 87 | 10.034 | 37.270 | 20.811 | 1.00 | 17.86 | C |
| ATOM | 709 | O | THR | H | 87 | 9.561 | 36.464 | 21.609 | 1.00 | 17.99 | O |
| ATOM | 710 | N | ALA | H | 88 | 9.622 | 37.369 | 19.547 | 1.00 | 17.47 | N |
| ATOM | 711 | CA | ALA | H | 88 | 8.513 | 36.550 | 19.029 | 1.00 | 17.13 | C |
| ATOM | 712 | CB | ALA | H | 88 | 7.194 | 36.965 | 19.690 | 1.00 | 16.80 | C |
| ATOM | 713 | C | ALA | H | 88 | 8.375 | 36.637 | 17.511 | 1.00 | 16.86 | C |
| ATOM | 714 | O | ALA | H | 88 | 8.962 | 37.518 | 16.871 | 1.00 | 17.38 | O |
| ATOM | 715 | N | MET | H | 89 | 7.603 | 35.719 | 16.937 | 1.00 | 16.34 | N |
| ATOM | 716 | CA | MET | H | 89 | 7.152 | 35.867 | 15.563 | 1.00 | 16.35 | C |
| ATOM | 717 | CB | MET | H | 89 | 6.899 | 34.507 | 14.905 | 1.00 | 16.52 | C |
| ATOM | 718 | CG | MET | H | 89 | 6.488 | 34.585 | 13.444 | 1.00 | 16.80 | C |
| ATOM | 719 | SD | MET | H | 89 | 7.853 | 35.068 | 12.350 | 1.00 | 22.00 | S |
| ATOM | 720 | CE | MET | H | 89 | 8.842 | 33.571 | 12.393 | 1.00 | 21.05 | C |
| ATOM | 721 | C | MET | H | 89 | 5.876 | 36.685 | 15.610 | 1.00 | 15.94 | C |
| ATOM | 722 | O | MET | H | 89 | 4.956 | 36.338 | 16.345 | 1.00 | 15.89 | O |
| ATOM | 723 | N | TYR | H | 90 | 5.836 | 37.771 | 14.843 | 1.00 | 15.54 | N |
| ATOM | 724 | CA | TYR | H | 90 | 4.694 | 38.683 | 14.832 | 1.00 | 15.55 | C |
| ATOM | 725 | CB | TYR | H | 90 | 5.160 | 40.135 | 15.030 | 1.00 | 15.01 | C |
| ATOM | 726 | CG | TYR | H | 90 | 5.600 | 40.441 | 16.435 | 1.00 | 14.58 | C |
| ATOM | 727 | CD1 | TYR | H | 90 | 6.833 | 39.998 | 16.910 | 1.00 | 13.92 | C |
| ATOM | 728 | CE1 | TYR | H | 90 | 7.236 | 40.264 | 18.195 | 1.00 | 13.42 | C |
| ATOM | 729 | CZ | TYR | H | 90 | 6.400 | 40.974 | 19.043 | 1.00 | 13.94 | C |
| ATOM | 730 | OH | TYR | H | 90 | 6.818 | 41.230 | 20.338 | 1.00 | 14.29 | O |
| ATOM | 731 | CE2 | TYR | H | 90 | 5.176 | 41.444 | 18.596 | 1.00 | 12.52 | C |
| ATOM | 732 | CD2 | TYR | H | 90 | 4.785 | 41.177 | 17.298 | 1.00 | 14.21 | C |
| ATOM | 733 | C | TYR | H | 90 | 3.916 | 38.548 | 13.523 | 1.00 | 15.96 | C |
| ATOM | 734 | O | TYR | H | 90 | 4.484 | 38.749 | 12.442 | 1.00 | 16.55 | O |
| ATOM | 735 | N | PHE | H | 91 | 2.629 | 38.210 | 13.625 | 1.00 | 15.64 | N |
| ATOM | 736 | CA | PHE | H | 91 | 1.771 | 38.044 | 12.456 | 1.00 | 15.42 | C |
| ATOM | 737 | CB | PHE | H | 91 | 0.975 | 36.735 | 12.537 | 1.00 | 15.48 | C |
| ATOM | 738 | CG | PHE | H | 91 | 1.807 | 35.498 | 12.494 | 1.00 | 16.17 | C |
| ATOM | 739 | CD1 | PHE | H | 91 | 2.273 | 35.001 | 11.281 | 1.00 | 18.05 | C |
| ATOM | 740 | CE1 | PHE | H | 91 | 3.054 | 33.843 | 11.230 | 1.00 | 19.08 | C |
| ATOM | 741 | CZ | PHE | H | 91 | 3.343 | 33.164 | 12.406 | 1.00 | 19.07 | C |
| ATOM | 742 | CE2 | PHE | H | 91 | 2.872 | 33.665 | 13.628 | 1.00 | 18.67 | C |
| ATOM | 743 | CD2 | PHE | H | 91 | 2.104 | 34.815 | 13.659 | 1.00 | 16.11 | C |
| ATOM | 744 | C | PHE | H | 91 | 0.742 | 39.158 | 12.353 | 1.00 | 15.39 | C |
| ATOM | 745 | O | PHE | H | 91 | 0.179 | 39.601 | 13.358 | 1.00 | 15.08 | O |
| ATOM | 746 | N | CYS | H | 92 | 0.464 | 39.580 | 11.128 | 1.00 | 15.19 | N |
| ATOM | 747 | CA | CYS | H | 92 | −0.765 | 40.299 | 10.875 | 1.00 | 15.58 | C |
| ATOM | 748 | CB | CYS | H | 92 | −0.535 | 41.537 | 10.004 | 1.00 | 16.07 | C |
| ATOM | 749 | SG | CYS | H | 92 | −0.173 | 41.216 | 8.293 | 1.00 | 18.89 | S |
| ATOM | 750 | C | CYS | H | 92 | −1.723 | 39.315 | 10.223 | 1.00 | 15.01 | C |
| ATOM | 751 | O | CYS | H | 92 | −1.293 | 38.330 | 9.601 | 1.00 | 14.87 | O |
| ATOM | 752 | N | ALA | H | 93 | −3.016 | 39.556 | 10.380 | 1.00 | 14.13 | N |
| ATOM | 753 | CA | ALA | H | 93 | −3.984 | 38.631 | 9.830 | 1.00 | 14.26 | C |
| ATOM | 754 | CB | ALA | H | 93 | −4.176 | 37.436 | 10.779 | 1.00 | 14.24 | C |
| ATOM | 755 | C | ALA | H | 93 | −5.317 | 39.291 | 9.507 | 1.00 | 14.49 | C |
| ATOM | 756 | O | ALA | H | 93 | −5.723 | 40.260 | 10.150 | 1.00 | 14.40 | O |
| ATOM | 757 | N | ARG | H | 94 | −5.990 | 38.766 | 8.492 | 1.00 | 14.68 | N |
| ATOM | 758 | CA | ARG | H | 94 | −7.366 | 39.159 | 8.229 | 1.00 | 15.05 | C |
| ATOM | 759 | CB | ARG | H | 94 | −7.502 | 39.854 | 6.868 | 1.00 | 15.31 | C |
| ATOM | 760 | CG | ARG | H | 94 | −8.924 | 40.216 | 6.493 | 1.00 | 15.77 | C |
| ATOM | 761 | CD | ARG | H | 94 | −8.940 | 41.167 | 5.332 | 1.00 | 17.38 | C |
| ATOM | 762 | NE | ARG | H | 94 | −10.313 | 41.497 | 4.967 | 1.00 | 20.94 | N |
| ATOM | 763 | CZ | ARG | H | 94 | −10.886 | 41.211 | 3.798 | 1.00 | 21.48 | C |
| ATOM | 764 | NH1 | ARG | H | 94 | −10.209 | 40.593 | 2.838 | 1.00 | 20.59 | N |
| ATOM | 765 | NH2 | ARG | H | 94 | −12.145 | 41.564 | 3.587 | 1.00 | 22.73 | N |
| ATOM | 766 | C | ARG | H | 94 | −8.245 | 37.917 | 8.329 | 1.00 | 14.61 | C |
| ATOM | 767 | O | ARG | H | 94 | −8.190 | 37.040 | 7.476 | 1.00 | 14.89 | O |
| ATOM | 768 | N | ARG | H | 95 | −9.039 | 37.870 | 9.394 | 1.00 | 14.34 | N |
| ATOM | 769 | CA | ARG | H | 95 | −9.837 | 36.707 | 9.755 | 1.00 | 14.28 | C |
| ATOM | 770 | CB | ARG | H | 95 | −9.485 | 36.250 | 11.176 | 1.00 | 13.79 | C |
| ATOM | 771 | CG | ARG | H | 95 | −8.015 | 35.911 | 11.395 | 1.00 | 12.32 | C |
| ATOM | 772 | CD | ARG | H | 95 | −7.842 | 35.051 | 12.650 | 1.00 | 12.74 | C |
| ATOM | 773 | NE | ARG | H | 95 | −8.173 | 35.768 | 13.890 | 1.00 | 13.91 | N |
| ATOM | 774 | CZ | ARG | H | 95 | −8.542 | 35.195 | 15.037 | 1.00 | 14.26 | C |
| ATOM | 775 | NH1 | ARG | H | 95 | −8.635 | 33.868 | 15.142 | 1.00 | 12.54 | N |
| ATOM | 776 | NH2 | ARG | H | 95 | −8.811 | 35.964 | 16.094 | 1.00 | 14.37 | N |
| ATOM | 777 | C | ARG | H | 95 | −11.354 | 36.951 | 9.667 | 1.00 | 14.83 | C |
| ATOM | 778 | O | ARG | H | 95 | −12.106 | 36.054 | 9.288 | 1.00 | 14.66 | O |
| ATOM | 779 | N | PHE | H | 96 | −11.797 | 38.158 | 10.016 | 1.00 | 15.51 | N |
| ATOM | 780 | CA | PHE | H | 96 | −13.230 | 38.457 | 10.095 | 1.00 | 16.12 | C |
| ATOM | 781 | CB | PHE | H | 96 | −13.650 | 38.670 | 11.561 | 1.00 | 15.96 | C |
| ATOM | 782 | CG | PHE | H | 96 | −13.143 | 37.614 | 12.504 | 1.00 | 14.44 | C |
| ATOM | 783 | CD1 | PHE | H | 96 | −13.598 | 36.304 | 12.418 | 1.00 | 14.52 | C |
| ATOM | 784 | CE1 | PHE | H | 96 | −13.114 | 35.315 | 13.286 | 1.00 | 14.23 | C |
| ATOM | 785 | CZ | PHE | H | 96 | −12.172 | 35.647 | 14.256 | 1.00 | 14.11 | C |
| ATOM | 786 | CE2 | PHE | H | 96 | −11.716 | 36.959 | 14.350 | 1.00 | 14.19 | C |
| ATOM | 787 | CD2 | PHE | H | 96 | −12.211 | 37.933 | 13.477 | 1.00 | 14.21 | C |
| ATOM | 788 | C | PHE | H | 96 | −13.652 | 39.670 | 9.265 | 1.00 | 16.80 | C |
| ATOM | 789 | O | PHE | H | 96 | −12.822 | 40.418 | 8.758 | 1.00 | 17.28 | O |
| ATOM | 790 | N | GLY | H | 97 | −14.959 | 39.835 | 9.116 | 1.00 | 17.64 | N |
| ATOM | 791 | CA | GLY | H | 97 | −15.560 | 41.091 | 8.671 | 1.00 | 18.09 | C |
| ATOM | 792 | C | GLY | H | 97 | −16.402 | 41.533 | 9.848 | 1.00 | 18.51 | C |
| ATOM | 793 | O | GLY | H | 97 | −16.329 | 40.919 | 10.912 | 1.00 | 18.94 | O |
| ATOM | 794 | N | TYR | H | 98 | −17.188 | 42.592 | 9.680 | 1.00 | 18.70 | N |
| ATOM | 795 | CA | TYR | H | 98 | −18.140 | 43.008 | 10.711 | 1.00 | 19.16 | C |
| ATOM | 796 | CB | TYR | H | 98 | −19.002 | 44.176 | 10.212 | 1.00 | 19.34 | C |
| ATOM | 797 | CG | TYR | H | 98 | −18.196 | 45.264 | 9.549 | 1.00 | 22.29 | C |
| ATOM | 798 | CD1 | TYR | H | 98 | −18.144 | 45.373 | 8.148 | 1.00 | 24.13 | C |
| ATOM | 799 | CE1 | TYR | H | 98 | −17.381 | 46.370 | 7.527 | 1.00 | 25.77 | C |
| ATOM | 800 | CZ | TYR | H | 98 | −16.644 | 47.269 | 8.308 | 1.00 | 26.49 | C |
| ATOM | 801 | OH | TYR | H | 98 | −15.886 | 48.259 | 7.705 | 1.00 | 25.96 | O |
| ATOM | 802 | CE2 | TYR | H | 98 | −16.675 | 47.174 | 9.697 | 1.00 | 25.91 | C |
| ATOM | 803 | CD2 | TYR | H | 98 | −17.450 | 46.170 | 10.310 | 1.00 | 24.16 | C |
| ATOM | 804 | C | TYR | H | 98 | −19.030 | 41.830 | 11.142 | 1.00 | 18.78 | C |
| ATOM | 805 | O | TYR | H | 98 | −19.297 | 40.908 | 10.364 | 1.00 | 18.57 | O |
| ATOM | 806 | N | TYR | H | 99 | −19.472 | 41.865 | 12.389 | 1.00 | 18.45 | N |
| ATOM | 807 | CA | TYR | H | 99 | −20.404 | 40.878 | 12.897 | 1.00 | 18.25 | C |
| ATOM | 808 | CB | TYR | H | 99 | −20.801 | 41.234 | 14.322 | 1.00 | 17.77 | C |
| ATOM | 809 | CG | TYR | H | 99 | −19.729 | 40.985 | 15.354 | 1.00 | 17.27 | C |
| ATOM | 810 | CD1 | TYR | H | 99 | −19.317 | 39.682 | 15.662 | 1.00 | 16.20 | C |
| ATOM | 811 | CE1 | TYR | H | 99 | −18.347 | 39.441 | 16.627 | 1.00 | 15.91 | C |
| ATOM | 812 | CZ | TYR | H | 99 | −17.781 | 40.510 | 17.302 | 1.00 | 17.39 | C |
| ATOM | 813 | OH | TYR | H | 99 | −16.819 | 40.274 | 18.262 | 1.00 | 18.93 | O |
| ATOM | 814 | CE2 | TYR | H | 99 | −18.181 | 41.817 | 17.024 | 1.00 | 16.83 | C |
| ATOM | 815 | CD2 | TYR | H | 99 | −19.152 | 42.044 | 16.054 | 1.00 | 16.57 | C |
| ATOM | 816 | C | TYR | H | 99 | −21.639 | 40.892 | 12.032 | 1.00 | 18.45 | C |
| ATOM | 817 | O | TYR | H | 99 | −22.096 | 41.967 | 11.639 | 1.00 | 19.49 | O |
| ATOM | 818 | N | GLY | H | 100 | −22.182 | 39.720 | 11.732 | 1.00 | 18.24 | N |
| ATOM | 819 | CA | GLY | H | 100 | −23.354 | 39.636 | 10.878 | 1.00 | 18.19 | C |
| ATOM | 820 | C | GLY | H | 100 | −23.078 | 39.604 | 9.376 | 1.00 | 18.25 | C |
| ATOM | 821 | O | GLY | H | 100 | −24.004 | 39.453 | 8.580 | 1.00 | 18.60 | O |
| ATOM | 822 | N | SER | H | 100A | −21.818 | 39.703 | 8.974 | 1.00 | 18.10 | N |
| ATOM | 823 | CA | SER | H | 100A | −21.496 | 39.786 | 7.548 | 1.00 | 17.99 | C |
| ATOM | 824 | CB | SER | H | 100A | −20.180 | 40.554 | 7.319 | 1.00 | 18.56 | C |
| ATOM | 825 | OG | SER | H | 100A | −19.038 | 39.863 | 7.824 | 1.00 | 18.77 | O |
| ATOM | 826 | C | SER | H | 100A | −21.430 | 38.445 | 6.841 | 1.00 | 17.88 | C |
| ATOM | 827 | O | SER | H | 100A | −21.423 | 38.403 | 5.613 | 1.00 | 17.85 | O |
| ATOM | 828 | N | GLY | H | 100B | −21.363 | 37.356 | 7.612 | 1.00 | 17.91 | N |
| ATOM | 829 | CA | GLY | H | 100B | −21.122 | 36.018 | 7.068 | 1.00 | 17.53 | C |
| ATOM | 830 | C | GLY | H | 100B | −19.680 | 35.787 | 6.625 | 1.00 | 17.90 | C |
| ATOM | 831 | O | GLY | H | 100B | −19.340 | 34.701 | 6.136 | 1.00 | 18.29 | O |
| ATOM | 832 | N | ASN | H | 100C | −18.818 | 36.792 | 6.785 | 1.00 | 17.36 | N |
| ATOM | 833 | CA | ASN | H | 100C | −17.447 | 36.679 | 6.296 | 1.00 | 17.20 | C |
| ATOM | 834 | CB | ASN | H | 100C | −16.905 | 38.037 | 5.883 | 1.00 | 17.80 | C |
| ATOM | 835 | CG | ASN | H | 100C | −17.510 | 38.536 | 4.575 | 1.00 | 19.98 | C |
| ATOM | 836 | OD1 | ASN | H | 100C | −17.839 | 37.743 | 3.677 | 1.00 | 21.86 | O |
| ATOM | 837 | ND2 | ASN | H | 100C | −17.659 | 39.857 | 4.459 | 1.00 | 19.25 | N |
| ATOM | 838 | C | ASN | H | 100C | −16.450 | 35.970 | 7.226 | 1.00 | 16.60 | C |
| ATOM | 839 | O | ASN | H | 100C | −16.298 | 36.310 | 8.413 | 1.00 | 16.17 | O |
| ATOM | 840 | N | TYR | H | 100D | −15.793 | 34.965 | 6.662 | 1.00 | 15.38 | N |
| ATOM | 841 | CA | TYR | H | 100D | −14.682 | 34.286 | 7.307 | 1.00 | 14.39 | C |
| ATOM | 842 | CB | TYR | H | 100D | −15.128 | 32.956 | 7.975 | 1.00 | 14.10 | C |
| ATOM | 843 | CG | TYR | H | 100D | −16.088 | 33.225 | 9.114 | 1.00 | 12.69 | C |
| ATOM | 844 | CD1 | TYR | H | 100D | −15.636 | 33.348 | 10.426 | 1.00 | 10.14 | C |
| ATOM | 845 | CE1 | TYR | H | 100D | −16.522 | 33.653 | 11.468 | 1.00 | 10.94 | C |
| ATOM | 846 | CZ | TYR | H | 100D | −17.867 | 33.849 | 11.184 | 1.00 | 12.40 | C |
| ATOM | 847 | OH | TYR | H | 100D | −18.763 | 34.159 | 12.183 | 1.00 | 14.90 | O |
| ATOM | 848 | CE2 | TYR | H | 100D | −18.337 | 33.729 | 9.890 | 1.00 | 12.41 | C |
| ATOM | 849 | CD2 | TYR | H | 100D | −17.449 | 33.429 | 8.862 | 1.00 | 12.75 | C |
| ATOM | 850 | C | TYR | H | 100D | −13.529 | 34.123 | 6.317 | 1.00 | 14.07 | C |
| ATOM | 851 | O | TYR | H | 100D | −13.670 | 33.508 | 5.255 | 1.00 | 13.30 | O |
| ATOM | 852 | N | PHE | H | 100E | −12.400 | 34.724 | 6.688 | 1.00 | 14.10 | N |
| ATOM | 853 | CA | PHE | H | 100E | −11.180 | 34.704 | 5.904 | 1.00 | 14.41 | C |
| ATOM | 854 | CB | PHE | H | 100E | −10.732 | 36.127 | 5.625 | 1.00 | 14.02 | C |
| ATOM | 855 | CG | PHE | H | 100E | −11.812 | 37.009 | 5.077 | 1.00 | 16.02 | C |
| ATOM | 856 | CD1 | PHE | H | 100E | −12.364 | 36.766 | 3.810 | 1.00 | 17.90 | C |
| ATOM | 857 | CE1 | PHE | H | 100E | −13.368 | 37.600 | 3.289 | 1.00 | 17.74 | C |
| ATOM | 858 | CZ | PHE | H | 100E | −13.809 | 38.686 | 4.031 | 1.00 | 17.20 | C |
| ATOM | 859 | CE2 | PHE | H | 100E | −13.259 | 38.935 | 5.286 | 1.00 | 16.36 | C |
| ATOM | 860 | CD2 | PHE | H | 100E | −12.271 | 38.096 | 5.806 | 1.00 | 14.98 | C |
| ATOM | 861 | C | PHE | H | 100E | −10.092 | 33.993 | 6.686 | 1.00 | 14.74 | C |
| ATOM | 862 | O | PHE | H | 100E | −10.224 | 33.760 | 7.891 | 1.00 | 14.38 | O |
| ATOM | 863 | N | ASP | H | 101 | −9.008 | 33.658 | 6.001 | 1.00 | 15.03 | N |
| ATOM | 864 | CA | ASP | H | 101 | −7.862 | 33.057 | 6.674 | 1.00 | 15.50 | C |
| ATOM | 865 | CB | ASP | H | 101 | −7.918 | 31.511 | 6.600 | 1.00 | 14.82 | C |
| ATOM | 866 | CG | ASP | H | 101 | −7.048 | 30.835 | 7.662 | 1.00 | 15.19 | C |
| ATOM | 867 | OD1 | ASP | H | 101 | −6.810 | 31.411 | 8.748 | 1.00 | 12.12 | O |
| ATOM | 868 | OD2 | ASP | H | 101 | −6.590 | 29.704 | 7.408 | 1.00 | 18.20 | O |
| ATOM | 869 | C | ASP | H | 101 | −6.586 | 33.600 | 6.056 | 1.00 | 15.95 | C |
| ATOM | 870 | O | ASP | H | 101 | −5.739 | 32.836 | 5.595 | 1.00 | 16.08 | O |
| ATOM | 871 | N | TYR | H | 102 | −6.473 | 34.929 | 6.019 | 1.00 | 16.50 | N |
| ATOM | 872 | CA | TYR | H | 102 | −5.302 | 35.576 | 5.446 | 1.00 | 16.62 | C |
| ATOM | 873 | CB | TYR | H | 102 | −5.700 | 36.825 | 4.665 | 1.00 | 16.89 | C |
| ATOM | 874 | CG | TYR | H | 102 | −6.704 | 36.574 | 3.558 | 1.00 | 18.79 | C |
| ATOM | 875 | CD1 | TYR | H | 102 | −6.388 | 35.748 | 2.474 | 1.00 | 21.74 | C |
| ATOM | 876 | CE1 | TYR | H | 102 | −7.313 | 35.519 | 1.442 | 1.00 | 22.94 | C |
| ATOM | 877 | CZ | TYR | H | 102 | −8.562 | 36.136 | 1.497 | 1.00 | 23.35 | C |
| ATOM | 878 | OH | TYR | H | 102 | −9.481 | 35.908 | 0.486 | 1.00 | 24.66 | O |
| ATOM | 879 | CE2 | TYR | H | 102 | −8.883 | 36.974 | 2.556 | 1.00 | 20.25 | C |
| ATOM | 880 | CD2 | TYR | H | 102 | −7.955 | 37.189 | 3.572 | 1.00 | 19.88 | C |
| ATOM | 881 | C | TYR | H | 102 | −4.310 | 35.924 | 6.544 | 1.00 | 16.63 | C |
| ATOM | 882 | O | TYR | H | 102 | −4.640 | 36.640 | 7.488 | 1.00 | 17.05 | O |
| ATOM | 883 | N | TRP | H | 103 | −3.102 | 35.391 | 6.423 | 1.00 | 16.56 | N |
| ATOM | 884 | CA | TRP | H | 103 | −2.031 | 35.627 | 7.380 | 1.00 | 16.39 | C |
| ATOM | 885 | CB | TRP | H | 103 | −1.635 | 34.322 | 8.085 | 1.00 | 15.79 | C |
| ATOM | 886 | CG | TRP | H | 103 | −2.700 | 33.786 | 8.972 | 1.00 | 13.87 | C |
| ATOM | 887 | CD1 | TRP | H | 103 | −3.858 | 33.171 | 8.588 | 1.00 | 12.41 | C |
| ATOM | 888 | NE1 | TRP | H | 103 | −4.600 | 32.834 | 9.695 | 1.00 | 11.83 | N |
| ATOM | 889 | CE2 | TRP | H | 103 | −3.923 | 33.225 | 10.818 | 1.00 | 11.49 | C |
| ATOM | 890 | CD2 | TRP | H | 103 | −2.719 | 33.822 | 10.400 | 1.00 | 11.66 | C |
| ATOM | 891 | CE3 | TRP | H | 103 | −1.835 | 34.301 | 11.366 | 1.00 | 10.51 | C |
| ATOM | 892 | CZ3 | TRP | H | 103 | −2.171 | 34.165 | 12.711 | 1.00 | 11.37 | C |
| ATOM | 893 | CH2 | TRP | H | 103 | −3.380 | 33.561 | 13.100 | 1.00 | 11.97 | C |
| ATOM | 894 | CZ2 | TRP | H | 103 | −4.266 | 33.090 | 12.172 | 1.00 | 12.62 | C |
| ATOM | 895 | C | TRP | H | 103 | −0.836 | 36.121 | 6.608 | 1.00 | 17.24 | C |
| ATOM | 896 | O | TRP | H | 103 | −0.566 | 35.655 | 5.500 | 1.00 | 17.59 | O |
| ATOM | 897 | N | GLY | H | 104 | −0.098 | 37.047 | 7.193 | 1.00 | 17.45 | N |
| ATOM | 898 | CA | GLY | H | 104 | 1.173 | 37.408 | 6.620 | 1.00 | 18.12 | C |
| ATOM | 899 | C | GLY | H | 104 | 2.139 | 36.283 | 6.886 | 1.00 | 18.59 | C |
| ATOM | 900 | O | GLY | H | 104 | 1.790 | 35.297 | 7.521 | 1.00 | 19.09 | O |
| ATOM | 901 | N | GLN | H | 105 | 3.361 | 36.428 | 6.397 | 1.00 | 18.95 | N |
| ATOM | 902 | CA | GLN | H | 105 | 4.351 | 35.392 | 6.544 | 1.00 | 19.41 | C |
| ATOM | 903 | CB | GLN | H | 105 | 5.363 | 35.435 | 5.378 | 1.00 | 19.45 | C |
| ATOM | 904 | CG | GLN | H | 105 | 6.432 | 36.544 | 5.446 | 1.00 | 20.58 | C |
| ATOM | 905 | CD | GLN | H | 105 | 5.930 | 37.925 | 5.006 | 1.00 | 22.61 | C |
| ATOM | 906 | OE1 | GLN | H | 105 | 4.725 | 38.175 | 4.945 | 1.00 | 24.10 | O |
| ATOM | 907 | NE2 | GLN | H | 105 | 6.866 | 38.830 | 4.702 | 1.00 | 22.41 | N |
| ATOM | 908 | C | GLN | H | 105 | 5.029 | 35.506 | 7.913 | 1.00 | 19.58 | C |
| ATOM | 909 | O | GLN | H | 105 | 5.881 | 34.688 | 8.254 | 1.00 | 19.68 | O |
| ATOM | 910 | N | GLY | H | 106 | 4.641 | 36.519 | 8.685 | 1.00 | 19.55 | N |
| ATOM | 911 | CA | GLY | H | 106 | 5.223 | 36.761 | 10.004 | 1.00 | 20.11 | C |
| ATOM | 912 | C | GLY | H | 106 | 6.518 | 37.549 | 9.968 | 1.00 | 20.84 | C |
| ATOM | 913 | O | GLY | H | 106 | 7.336 | 37.395 | 9.047 | 1.00 | 21.29 | O |
| ATOM | 914 | N | THR | H | 107 | 6.713 | 38.408 | 10.964 | 1.00 | 21.06 | N |
| ATOM | 915 | CA | THR | H | 107 | 7.960 | 39.153 | 11.090 | 1.00 | 21.08 | C |
| ATOM | 916 | CB | THR | H | 107 | 7.700 | 40.680 | 11.080 | 1.00 | 20.98 | C |
| ATOM | 917 | OG1 | THR | H | 107 | 7.042 | 41.047 | 9.870 | 1.00 | 21.39 | O |
| ATOM | 918 | CG2 | THR | H | 107 | 8.988 | 41.470 | 11.187 | 1.00 | 20.82 | C |
| ATOM | 919 | C | THR | H | 107 | 8.661 | 38.730 | 12.385 | 1.00 | 21.36 | C |
| ATOM | 920 | O | THR | H | 107 | 8.160 | 39.000 | 13.479 | 1.00 | 21.48 | O |
| ATOM | 921 | N | MET | H | 108 | 9.816 | 38.078 | 12.260 | 1.00 | 21.49 | N |
| ATOM | 922 | CA | MET | H | 108 | 10.574 | 37.653 | 13.436 | 1.00 | 22.23 | C |
| ATOM | 923 | CB | MET | H | 108 | 11.622 | 36.584 | 13.085 | 1.00 | 22.73 | C |
| ATOM | 924 | CG | MET | H | 108 | 12.242 | 35.906 | 14.304 | 1.00 | 24.87 | C |
| ATOM | 925 | SD | MET | H | 108 | 10.992 | 35.079 | 15.343 | 1.00 | 32.15 | S |
| ATOM | 926 | CE | MET | H | 108 | 11.647 | 35.355 | 17.001 | 1.00 | 29.99 | C |
| ATOM | 927 | C | MET | H | 108 | 11.233 | 38.833 | 14.134 | 1.00 | 21.95 | C |
| ATOM | 928 | O | MET | H | 108 | 12.045 | 39.529 | 13.553 | 1.00 | 21.98 | O |
| ATOM | 929 | N | VAL | H | 109 | 10.864 | 39.053 | 15.389 | 1.00 | 21.90 | N |
| ATOM | 930 | CA | VAL | H | 109 | 11.484 | 40.090 | 16.196 | 1.00 | 21.22 | C |
| ATOM | 931 | CB | VAL | H | 109 | 10.428 | 41.018 | 16.812 | 1.00 | 21.09 | C |
| ATOM | 932 | CG1 | VAL | H | 109 | 11.069 | 42.007 | 17.757 | 1.00 | 19.61 | C |
| ATOM | 933 | CG2 | VAL | H | 109 | 9.632 | 41.727 | 15.709 | 1.00 | 20.13 | C |
| ATOM | 934 | C | VAL | H | 109 | 12.335 | 39.453 | 17.287 | 1.00 | 21.70 | C |
| ATOM | 935 | O | VAL | H | 109 | 11.836 | 38.668 | 18.080 | 1.00 | 21.80 | O |
| ATOM | 936 | N | THR | H | 110 | 13.629 | 39.778 | 17.286 | 1.00 | 22.43 | N |
| ATOM | 937 | CA | THR | H | 110 | 14.575 | 39.363 | 18.327 | 1.00 | 22.91 | C |
| ATOM | 938 | CB | THR | H | 110 | 15.825 | 38.687 | 17.713 | 1.00 | 23.16 | C |
| ATOM | 939 | OG1 | THR | H | 110 | 15.423 | 37.607 | 16.856 | 1.00 | 23.19 | O |
| ATOM | 940 | CG2 | THR | H | 110 | 16.775 | 38.170 | 18.806 | 1.00 | 22.54 | C |
| ATOM | 941 | C | THR | H | 110 | 15.013 | 40.599 | 19.124 | 1.00 | 23.34 | C |
| ATOM | 942 | O | THR | H | 110 | 15.673 | 41.497 | 18.593 | 1.00 | 23.41 | O |
| ATOM | 943 | N | VAL | H | 111 | 14.619 | 40.641 | 20.392 | 1.00 | 23.80 | N |
| ATOM | 944 | CA | VAL | H | 111 | 15.050 | 41.683 | 21.305 | 1.00 | 24.11 | C |
| ATOM | 945 | CB | VAL | H | 111 | 13.893 | 42.203 | 22.193 | 1.00 | 24.27 | C |
| ATOM | 946 | CG1 | VAL | H | 111 | 14.381 | 43.350 | 23.084 | 1.00 | 23.63 | C |
| ATOM | 947 | CG2 | VAL | H | 111 | 12.702 | 42.650 | 21.339 | 1.00 | 23.57 | C |
| ATOM | 948 | C | VAL | H | 111 | 16.137 | 41.100 | 22.188 | 1.00 | 24.68 | C |
| ATOM | 949 | O | VAL | H | 111 | 15.891 | 40.160 | 22.948 | 1.00 | 24.63 | O |
| ATOM | 950 | N | SER | H | 112 | 17.342 | 41.653 | 22.076 | 1.00 | 25.45 | N |
| ATOM | 951 | CA | SER | H | 112 | 18.470 | 41.206 | 22.887 | 1.00 | 26.27 | C |
| ATOM | 952 | CB | SER | H | 112 | 19.074 | 39.933 | 22.314 | 1.00 | 25.98 | C |
| ATOM | 953 | OG | SER | H | 112 | 20.149 | 39.512 | 23.116 | 1.00 | 25.20 | O |
| ATOM | 954 | C | SER | H | 112 | 19.565 | 42.245 | 22.990 | 1.00 | 27.28 | C |
| ATOM | 955 | O | SER | H | 112 | 19.738 | 43.068 | 22.092 | 1.00 | 27.35 | O |
| ATOM | 956 | N | SER | H | 113 | 20.325 | 42.168 | 24.084 | 1.00 | 28.65 | N |
| ATOM | 957 | CA | SER | H | 113 | 21.452 | 43.069 | 24.339 | 1.00 | 29.53 | C |
| ATOM | 958 | CB | SER | H | 113 | 21.738 | 43.125 | 25.842 | 1.00 | 29.77 | C |
| ATOM | 959 | OG | SER | H | 113 | 22.416 | 41.952 | 26.277 | 1.00 | 30.84 | O |
| ATOM | 960 | C | SER | H | 113 | 22.720 | 42.655 | 23.597 | 1.00 | 29.91 | C |
| ATOM | 961 | O | SER | H | 113 | 23.776 | 43.234 | 23.810 | 1.00 | 30.25 | O |
| ATOM | 962 | N | ALA | H | 114 | 22.612 | 41.658 | 22.728 | 1.00 | 30.62 | N |
| ATOM | 963 | CA | ALA | H | 114 | 23.778 | 41.046 | 22.099 | 1.00 | 31.17 | C |
| ATOM | 964 | CB | ALA | H | 114 | 23.596 | 39.522 | 22.020 | 1.00 | 31.17 | C |
| ATOM | 965 | C | ALA | H | 114 | 24.064 | 41.637 | 20.721 | 1.00 | 31.63 | C |
| ATOM | 966 | O | ALA | H | 114 | 23.243 | 42.382 | 20.179 | 1.00 | 32.13 | O |
| ATOM | 967 | N | SER | H | 115 | 25.222 | 41.292 | 20.159 | 1.00 | 31.90 | N |
| ATOM | 968 | CA | SER | H | 115 | 25.684 | 41.856 | 18.892 | 1.00 | 32.43 | C |
| ATOM | 969 | CB | SER | H | 115 | 26.990 | 42.630 | 19.097 | 1.00 | 32.52 | C |
| ATOM | 970 | OG | SER | H | 115 | 26.838 | 43.617 | 20.097 | 1.00 | 33.33 | O |
| ATOM | 971 | C | SER | H | 115 | 25.925 | 40.767 | 17.866 | 1.00 | 32.57 | C |
| ATOM | 972 | O | SER | H | 115 | 26.297 | 39.647 | 18.218 | 1.00 | 32.69 | O |
| ATOM | 973 | N | THR | H | 116 | 25.737 | 41.117 | 16.597 | 1.00 | 32.79 | N |
| ATOM | 974 | CA | THR | H | 116 | 25.922 | 40.188 | 15.491 | 1.00 | 33.11 | C |
| ATOM | 975 | CB | THR | H | 116 | 25.732 | 40.893 | 14.137 | 1.00 | 33.28 | C |
| ATOM | 976 | OG1 | THR | H | 116 | 24.555 | 41.708 | 14.186 | 1.00 | 33.19 | O |
| ATOM | 977 | CG2 | THR | H | 116 | 25.584 | 39.872 | 13.009 | 1.00 | 33.87 | C |
| ATOM | 978 | C | THR | H | 116 | 27.293 | 39.510 | 15.561 | 1.00 | 33.18 | C |
| ATOM | 979 | O | THR | H | 116 | 28.323 | 40.172 | 15.663 | 1.00 | 33.30 | O |
| ATOM | 980 | N | LYS | H | 117 | 27.276 | 38.184 | 15.524 | 1.00 | 33.28 | N |
| ATOM | 981 | CA | LYS | H | 117 | 28.469 | 37.364 | 15.677 | 1.00 | 33.53 | C |
| ATOM | 982 | CB | LYS | H | 117 | 28.708 | 37.046 | 17.164 | 1.00 | 33.95 | C |
| ATOM | 983 | CG | LYS | H | 117 | 30.020 | 36.302 | 17.444 | 1.00 | 35.48 | C |
| ATOM | 984 | CD | LYS | H | 117 | 30.257 | 36.054 | 18.944 | 1.00 | 37.51 | C |
| ATOM | 985 | CE | LYS | H | 117 | 31.514 | 35.191 | 19.184 | 1.00 | 38.49 | C |
| ATOM | 986 | NZ | LYS | H | 117 | 31.444 | 33.820 | 18.534 | 1.00 | 38.84 | N |
| ATOM | 987 | C | LYS | H | 117 | 28.310 | 36.063 | 14.888 | 1.00 | 33.05 | C |
| ATOM | 988 | O | LYS | H | 117 | 27.269 | 35.400 | 14.963 | 1.00 | 33.23 | O |
| ATOM | 989 | N | GLY | H | 118 | 29.338 | 35.707 | 14.129 | 1.00 | 32.39 | N |
| ATOM | 990 | CA | GLY | H | 118 | 29.345 | 34.450 | 13.392 | 1.00 | 31.50 | C |
| ATOM | 991 | C | GLY | H | 118 | 29.695 | 33.305 | 14.326 | 1.00 | 31.07 | C |
| ATOM | 992 | O | GLY | H | 118 | 30.338 | 33.519 | 15.360 | 1.00 | 31.28 | O |
| ATOM | 993 | N | PRO | H | 119 | 29.273 | 32.080 | 13.972 | 1.00 | 30.52 | N |
| ATOM | 994 | CA | PRO | H | 119 | 29.489 | 30.922 | 14.832 | 1.00 | 30.41 | C |
| ATOM | 995 | CB | PRO | H | 119 | 28.556 | 29.873 | 14.225 | 1.00 | 30.29 | C |
| ATOM | 996 | CG | PRO | H | 119 | 28.522 | 30.218 | 12.784 | 1.00 | 29.96 | C |
| ATOM | 997 | CD | PRO | H | 119 | 28.536 | 31.717 | 12.748 | 1.00 | 30.27 | C |
| ATOM | 998 | C | PRO | H | 119 | 30.914 | 30.399 | 14.769 | 1.00 | 30.18 | C |
| ATOM | 999 | O | PRO | H | 119 | 31.650 | 30.736 | 13.846 | 1.00 | 30.21 | O |
| ATOM | 1000 | N | SER | H | 120 | 31.291 | 29.583 | 15.748 | 1.00 | 29.89 | N |
| ATOM | 1001 | CA | SER | H | 120 | 32.445 | 28.706 | 15.595 | 1.00 | 29.81 | C |
| ATOM | 1002 | CB | SER | H | 120 | 33.335 | 28.736 | 16.840 | 1.00 | 29.98 | C |
| ATOM | 1003 | OG | SER | H | 120 | 33.792 | 30.060 | 17.124 | 1.00 | 29.42 | O |
| ATOM | 1004 | C | SER | H | 120 | 31.933 | 27.297 | 15.287 | 1.00 | 29.84 | C |
| ATOM | 1005 | O | SER | H | 120 | 30.900 | 26.874 | 15.813 | 1.00 | 30.10 | O |
| ATOM | 1006 | N | VAL | H | 121 | 32.634 | 26.580 | 14.411 | 1.00 | 29.53 | N |
| ATOM | 1007 | CA | VAL | H | 121 | 32.179 | 25.268 | 13.978 | 1.00 | 28.95 | C |
| ATOM | 1008 | CB | VAL | H | 121 | 31.907 | 25.234 | 12.440 | 1.00 | 28.82 | C |
| ATOM | 1009 | CG1 | VAL | H | 121 | 31.412 | 23.866 | 11.998 | 1.00 | 27.87 | C |
| ATOM | 1010 | CG2 | VAL | H | 121 | 30.891 | 26.283 | 12.062 | 1.00 | 27.92 | C |
| ATOM | 1011 | C | VAL | H | 121 | 33.171 | 24.183 | 14.400 | 1.00 | 29.42 | C |
| ATOM | 1012 | O | VAL | H | 121 | 34.304 | 24.140 | 13.905 | 1.00 | 29.74 | O |
| ATOM | 1013 | N | PHE | H | 122 | 32.731 | 23.310 | 15.307 | 1.00 | 29.59 | N |
| ATOM | 1014 | CA | PHE | H | 122 | 33.562 | 22.240 | 15.856 | 1.00 | 29.95 | C |
| ATOM | 1015 | CB | PHE | H | 122 | 33.682 | 22.346 | 17.385 | 1.00 | 29.96 | C |
| ATOM | 1016 | CG | PHE | H | 122 | 34.090 | 23.705 | 17.881 | 1.00 | 30.40 | C |
| ATOM | 1017 | CD1 | PHE | H | 122 | 35.369 | 24.205 | 17.629 | 1.00 | 30.91 | C |
| ATOM | 1018 | CE1 | PHE | H | 122 | 35.746 | 25.472 | 18.092 | 1.00 | 29.57 | C |
| ATOM | 1019 | CZ | PHE | H | 122 | 34.844 | 26.222 | 18.815 | 1.00 | 30.02 | C |
| ATOM | 1020 | CE2 | PHE | H | 122 | 33.567 | 25.722 | 19.079 | 1.00 | 28.87 | C |
| ATOM | 1021 | CD2 | PHE | H | 122 | 33.202 | 24.480 | 18.621 | 1.00 | 29.07 | C |
| ATOM | 1022 | C | PHE | H | 122 | 32.966 | 20.883 | 15.501 | 1.00 | 30.33 | C |
| ATOM | 1023 | O | PHE | H | 122 | 31.750 | 20.739 | 15.505 | 1.00 | 30.07 | O |
| ATOM | 1024 | N | PRO | H | 123 | 33.824 | 19.882 | 15.199 | 1.00 | 30.75 | N |
| ATOM | 1025 | CA | PRO | H | 123 | 33.341 | 18.561 | 14.785 | 1.00 | 31.23 | C |
| ATOM | 1026 | CB | PRO | H | 123 | 34.529 | 17.984 | 14.012 | 1.00 | 31.26 | C |
| ATOM | 1027 | CG | PRO | H | 123 | 35.715 | 18.636 | 14.634 | 1.00 | 31.06 | C |
| ATOM | 1028 | CD | PRO | H | 123 | 35.281 | 20.019 | 15.005 | 1.00 | 30.28 | C |
| ATOM | 1029 | C | PRO | H | 123 | 32.972 | 17.618 | 15.923 | 1.00 | 31.83 | C |
| ATOM | 1030 | O | PRO | H | 123 | 33.644 | 17.569 | 16.942 | 1.00 | 31.97 | O |
| ATOM | 1031 | N | LEU | H | 124 | 31.893 | 16.878 | 15.725 | 1.00 | 32.79 | N |
| ATOM | 1032 | CA | LEU | H | 124 | 31.528 | 15.778 | 16.596 | 1.00 | 33.75 | C |
| ATOM | 1033 | CB | LEU | H | 124 | 30.024 | 15.813 | 16.906 | 1.00 | 33.37 | C |
| ATOM | 1034 | CG | LEU | H | 124 | 29.510 | 17.131 | 17.509 | 1.00 | 33.16 | C |
| ATOM | 1035 | CD1 | LEU | H | 124 | 28.016 | 17.324 | 17.279 | 1.00 | 32.02 | C |
| ATOM | 1036 | CD2 | LEU | H | 124 | 29.846 | 17.246 | 19.003 | 1.00 | 32.62 | C |
| ATOM | 1037 | C | LEU | H | 124 | 31.929 | 14.509 | 15.844 | 1.00 | 34.62 | C |
| ATOM | 1038 | O | LEU | H | 124 | 31.189 | 14.021 | 14.981 | 1.00 | 34.35 | O |
| ATOM | 1039 | N | ALA | H | 125 | 33.125 | 14.014 | 16.159 | 1.00 | 35.88 | N |
| ATOM | 1040 | CA | ALA | H | 125 | 33.778 | 12.948 | 15.402 | 1.00 | 37.35 | C |
| ATOM | 1041 | CB | ALA | H | 125 | 35.280 | 13.031 | 15.590 | 1.00 | 37.34 | C |
| ATOM | 1042 | C | ALA | H | 125 | 33.269 | 11.557 | 15.773 | 1.00 | 38.56 | C |
| ATOM | 1043 | O | ALA | H | 125 | 32.972 | 11.298 | 16.941 | 1.00 | 38.77 | O |
| ATOM | 1044 | N | PRO | H | 126 | 33.182 | 10.649 | 14.785 | 1.00 | 39.91 | N |
| ATOM | 1045 | CA | PRO | H | 126 | 32.671 | 9.307 | 15.049 | 1.00 | 41.27 | C |
| ATOM | 1046 | CB | PRO | H | 126 | 32.317 | 8.804 | 13.656 | 1.00 | 41.03 | C |
| ATOM | 1047 | CG | PRO | H | 126 | 33.318 | 9.476 | 12.758 | 1.00 | 40.31 | C |
| ATOM | 1048 | CD | PRO | H | 126 | 33.673 | 10.788 | 13.399 | 1.00 | 39.91 | C |
| ATOM | 1049 | C | PRO | H | 126 | 33.750 | 8.416 | 15.644 | 1.00 | 42.98 | C |
| ATOM | 1050 | O | PRO | H | 126 | 34.931 | 8.770 | 15.593 | 1.00 | 43.27 | O |
| ATOM | 1051 | N | SER | H | 127 | 33.346 | 7.266 | 16.187 | 1.00 | 44.89 | N |
| ATOM | 1052 | CA | SER | H | 127 | 34.288 | 6.259 | 16.695 | 1.00 | 46.36 | C |
| ATOM | 1053 | CB | SER | H | 127 | 34.929 | 6.732 | 18.003 | 1.00 | 46.44 | C |
| ATOM | 1054 | OG | SER | H | 127 | 33.926 | 7.148 | 18.918 | 1.00 | 47.49 | O |
| ATOM | 1055 | C | SER | H | 127 | 33.621 | 4.893 | 16.889 | 1.00 | 47.23 | C |
| ATOM | 1056 | O | SER | H | 127 | 32.657 | 4.555 | 16.189 | 1.00 | 47.40 | O |
| ATOM | 1057 | N | SER | H | 128 | 34.144 | 4.124 | 17.847 | 1.00 | 48.04 | N |
| ATOM | 1058 | CA | SER | H | 128 | 33.717 | 2.744 | 18.106 | 1.00 | 48.73 | C |
| ATOM | 1059 | CB | SER | H | 128 | 34.951 | 1.874 | 18.359 | 1.00 | 49.03 | C |
| ATOM | 1060 | OG | SER | H | 128 | 35.814 | 2.489 | 19.310 | 1.00 | 49.35 | O |
| ATOM | 1061 | C | SER | H | 128 | 32.740 | 2.614 | 19.287 | 1.00 | 48.87 | C |
| ATOM | 1062 | O | SER | H | 128 | 31.549 | 2.936 | 19.180 | 1.00 | 49.02 | O |
| ATOM | 1063 | N | GLY | H | 133 | 31.127 | −3.348 | 11.454 | 1.00 | 44.51 | N |
| ATOM | 1064 | CA | GLY | H | 133 | 30.289 | −2.921 | 10.337 | 1.00 | 44.45 | C |
| ATOM | 1065 | C | GLY | H | 133 | 28.821 | −2.744 | 10.717 | 1.00 | 44.30 | C |
| ATOM | 1066 | O | GLY | H | 133 | 27.946 | −3.428 | 10.162 | 1.00 | 44.47 | O |
| ATOM | 1067 | N | GLY | H | 134 | 28.563 | −1.838 | 11.671 | 1.00 | 43.64 | N |
| ATOM | 1068 | CA | GLY | H | 134 | 27.202 | −1.423 | 12.065 | 1.00 | 42.34 | C |
| ATOM | 1069 | C | GLY | H | 134 | 26.961 | 0.032 | 11.668 | 1.00 | 41.56 | C |
| ATOM | 1070 | O | GLY | H | 134 | 27.303 | 0.441 | 10.544 | 1.00 | 41.56 | O |
| ATOM | 1071 | N | THR | H | 135 | 26.384 | 0.821 | 12.577 | 1.00 | 40.20 | N |
| ATOM | 1072 | CA | THR | H | 135 | 26.142 | 2.240 | 12.288 | 1.00 | 38.91 | C |
| ATOM | 1073 | CB | THR | H | 135 | 24.635 | 2.580 | 12.143 | 1.00 | 39.32 | C |
| ATOM | 1074 | OG1 | THR | H | 135 | 23.892 | 1.940 | 13.184 | 1.00 | 40.22 | O |
| ATOM | 1075 | CG2 | THR | H | 135 | 24.098 | 2.110 | 10.774 | 1.00 | 39.99 | C |
| ATOM | 1076 | C | THR | H | 135 | 26.827 | 3.197 | 13.266 | 1.00 | 37.42 | C |
| ATOM | 1077 | O | THR | H | 135 | 26.819 | 2.987 | 14.483 | 1.00 | 37.48 | O |
| ATOM | 1078 | N | ALA | H | 136 | 27.415 | 4.246 | 12.694 | 1.00 | 35.28 | N |
| ATOM | 1079 | CA | ALA | H | 136 | 28.168 | 5.260 | 13.413 | 1.00 | 33.62 | C |
| ATOM | 1080 | CB | ALA | H | 136 | 29.570 | 5.386 | 12.812 | 1.00 | 33.54 | C |
| ATOM | 1081 | C | ALA | H | 136 | 27.451 | 6.615 | 13.356 | 1.00 | 32.38 | C |
| ATOM | 1082 | O | ALA | H | 136 | 26.655 | 6.873 | 12.445 | 1.00 | 32.01 | O |
| ATOM | 1083 | N | ALA | H | 137 | 27.744 | 7.479 | 14.318 | 1.00 | 30.60 | N |
| ATOM | 1084 | CA | ALA | H | 137 | 27.150 | 8.806 | 14.341 | 1.00 | 29.72 | C |
| ATOM | 1085 | CB | ALA | H | 137 | 26.252 | 8.978 | 15.549 | 1.00 | 29.69 | C |
| ATOM | 1086 | C | ALA | H | 137 | 28.247 | 9.834 | 14.352 | 1.00 | 28.84 | C |
| ATOM | 1087 | O | ALA | H | 137 | 29.290 | 9.604 | 14.946 | 1.00 | 28.78 | O |
| ATOM | 1088 | N | LEU | H | 138 | 28.008 | 10.956 | 13.677 | 1.00 | 28.16 | N |
| ATOM | 1089 | CA | LEU | H | 138 | 28.941 | 12.085 | 13.648 | 1.00 | 27.55 | C |
| ATOM | 1090 | CB | LEU | H | 138 | 29.975 | 11.883 | 12.543 | 1.00 | 27.61 | C |
| ATOM | 1091 | CG | LEU | H | 138 | 29.409 | 11.794 | 11.116 | 1.00 | 28.05 | C |
| ATOM | 1092 | CD1 | LEU | H | 138 | 29.658 | 13.095 | 10.362 | 1.00 | 26.98 | C |
| ATOM | 1093 | CD2 | LEU | H | 138 | 30.018 | 10.629 | 10.365 | 1.00 | 27.75 | C |
| ATOM | 1094 | C | LEU | H | 138 | 28.167 | 13.396 | 13.451 | 1.00 | 27.10 | C |
| ATOM | 1095 | O | LEU | H | 138 | 26.983 | 13.368 | 13.107 | 1.00 | 26.64 | O |
| ATOM | 1096 | N | GLY | H | 139 | 28.831 | 14.532 | 13.666 | 1.00 | 26.67 | N |
| ATOM | 1097 | CA | GLY | H | 139 | 28.167 | 15.822 | 13.578 | 1.00 | 26.45 | C |
| ATOM | 1098 | C | GLY | H | 139 | 29.062 | 17.043 | 13.600 | 1.00 | 26.82 | C |
| ATOM | 1099 | O | GLY | H | 139 | 30.281 | 16.940 | 13.534 | 1.00 | 26.78 | O |
| ATOM | 1100 | N | CYS | H | 140 | 28.431 | 18.211 | 13.670 | 1.00 | 27.23 | N |
| ATOM | 1101 | CA | CYS | H | 140 | 29.116 | 19.483 | 13.877 | 1.00 | 27.75 | C |
| ATOM | 1102 | CB | CYS | H | 140 | 29.089 | 20.342 | 12.613 | 1.00 | 27.91 | C |
| ATOM | 1103 | SG | CYS | H | 140 | 30.117 | 19.681 | 11.311 | 1.00 | 32.74 | S |
| ATOM | 1104 | C | CYS | H | 140 | 28.418 | 20.230 | 14.993 | 1.00 | 27.29 | C |
| ATOM | 1105 | O | CYS | H | 140 | 27.192 | 20.175 | 15.127 | 1.00 | 27.76 | O |
| ATOM | 1106 | N | LEU | H | 141 | 29.207 | 20.932 | 15.790 | 1.00 | 26.84 | N |
| ATOM | 1107 | CA | LEU | H | 141 | 28.694 | 21.807 | 16.805 | 1.00 | 26.34 | C |
| ATOM | 1108 | CB | LEU | H | 141 | 29.514 | 21.658 | 18.074 | 1.00 | 26.06 | C |
| ATOM | 1109 | CG | LEU | H | 141 | 29.214 | 22.610 | 19.229 | 1.00 | 25.34 | C |
| ATOM | 1110 | CD1 | LEU | H | 141 | 27.850 | 22.316 | 19.857 | 1.00 | 22.47 | C |
| ATOM | 1111 | CD2 | LEU | H | 141 | 30.328 | 22.512 | 20.260 | 1.00 | 23.98 | C |
| ATOM | 1112 | C | LEU | H | 141 | 28.799 | 23.225 | 16.281 | 1.00 | 26.65 | C |
| ATOM | 1113 | O | LEU | H | 141 | 29.877 | 23.668 | 15.879 | 1.00 | 27.06 | O |
| ATOM | 1114 | N | VAL | H | 142 | 27.675 | 23.930 | 16.280 | 1.00 | 26.47 | N |
| ATOM | 1115 | CA | VAL | H | 142 | 27.614 | 25.286 | 15.773 | 1.00 | 26.29 | C |
| ATOM | 1116 | CB | VAL | H | 142 | 26.541 | 25.422 | 14.681 | 1.00 | 26.08 | C |
| ATOM | 1117 | CG1 | VAL | H | 142 | 26.531 | 26.832 | 14.084 | 1.00 | 25.48 | C |
| ATOM | 1118 | CG2 | VAL | H | 142 | 26.785 | 24.408 | 13.604 | 1.00 | 25.76 | C |
| ATOM | 1119 | C | VAL | H | 142 | 27.343 | 26.194 | 16.961 | 1.00 | 26.77 | C |
| ATOM | 1120 | O | VAL | H | 142 | 26.213 | 26.274 | 17.461 | 1.00 | 26.81 | O |
| ATOM | 1121 | N | LYS | H | 143 | 28.395 | 26.878 | 17.403 | 1.00 | 27.01 | N |
| ATOM | 1122 | CA | LYS | H | 143 | 28.407 | 27.519 | 18.711 | 1.00 | 27.53 | C |
| ATOM | 1123 | CB | LYS | H | 143 | 29.488 | 26.865 | 19.584 | 1.00 | 27.45 | C |
| ATOM | 1124 | CG | LYS | H | 143 | 29.527 | 27.351 | 21.024 | 1.00 | 28.29 | C |
| ATOM | 1125 | CD | LYS | H | 143 | 30.106 | 26.292 | 21.952 | 1.00 | 29.59 | C |
| ATOM | 1126 | CE | LYS | H | 143 | 30.437 | 26.868 | 23.326 | 1.00 | 30.18 | C |
| ATOM | 1127 | NZ | LYS | H | 143 | 29.215 | 27.296 | 24.064 | 1.00 | 30.93 | N |
| ATOM | 1128 | C | LYS | H | 143 | 28.591 | 29.033 | 18.679 | 1.00 | 27.66 | C |
| ATOM | 1129 | O | LYS | H | 143 | 29.318 | 29.566 | 17.849 | 1.00 | 28.21 | O |
| ATOM | 1130 | N | ASP | H | 144 | 27.914 | 29.714 | 19.598 | 1.00 | 27.77 | N |
| ATOM | 1131 | CA | ASP | H | 144 | 28.156 | 31.121 | 19.882 | 1.00 | 27.38 | C |
| ATOM | 1132 | CB | ASP | H | 144 | 29.576 | 31.323 | 20.443 | 1.00 | 27.46 | C |
| ATOM | 1133 | CG | ASP | H | 144 | 29.767 | 30.731 | 21.842 | 1.00 | 28.10 | C |
| ATOM | 1134 | OD1 | ASP | H | 144 | 28.808 | 30.210 | 22.459 | 1.00 | 28.02 | O |
| ATOM | 1135 | OD2 | ASP | H | 144 | 30.916 | 30.786 | 22.331 | 1.00 | 29.69 | O |
| ATOM | 1136 | C | ASP | H | 144 | 27.923 | 32.023 | 18.671 | 1.00 | 27.12 | C |
| ATOM | 1137 | O | ASP | H | 144 | 28.826 | 32.751 | 18.242 | 1.00 | 27.32 | O |
| ATOM | 1138 | N | TYR | H | 145 | 26.715 | 31.975 | 18.118 | 1.00 | 26.62 | N |
| ATOM | 1139 | CA | TYR | H | 145 | 26.336 | 32.900 | 17.054 | 1.00 | 26.03 | C |
| ATOM | 1140 | CB | TYR | H | 145 | 26.070 | 32.177 | 15.736 | 1.00 | 25.88 | C |
| ATOM | 1141 | CG | TYR | H | 145 | 24.886 | 31.248 | 15.763 | 1.00 | 26.72 | C |
| ATOM | 1142 | CD1 | TYR | H | 145 | 25.051 | 29.889 | 16.039 | 1.00 | 26.70 | C |
| ATOM | 1143 | CE1 | TYR | H | 145 | 23.978 | 29.034 | 16.063 | 1.00 | 26.51 | C |
| ATOM | 1144 | CZ | TYR | H | 145 | 22.717 | 29.530 | 15.804 | 1.00 | 27.81 | C |
| ATOM | 1145 | OH | TYR | H | 145 | 21.629 | 28.680 | 15.822 | 1.00 | 29.75 | O |
| ATOM | 1146 | CE2 | TYR | H | 145 | 22.526 | 30.876 | 15.531 | 1.00 | 27.36 | C |
| ATOM | 1147 | CD2 | TYR | H | 145 | 23.600 | 31.722 | 15.511 | 1.00 | 26.33 | C |
| ATOM | 1148 | C | TYR | H | 145 | 25.130 | 33.724 | 17.441 | 1.00 | 25.86 | C |
| ATOM | 1149 | O | TYR | H | 145 | 24.387 | 33.369 | 18.358 | 1.00 | 26.24 | O |
| ATOM | 1150 | N | PHE | H | 146 | 24.947 | 34.826 | 16.724 | 1.00 | 25.39 | N |
| ATOM | 1151 | CA | PHE | H | 146 | 23.825 | 35.717 | 16.926 | 1.00 | 25.04 | C |
| ATOM | 1152 | CB | PHE | H | 146 | 24.034 | 36.558 | 18.182 | 1.00 | 24.33 | C |
| ATOM | 1153 | CG | PHE | H | 146 | 22.906 | 37.491 | 18.466 | 1.00 | 23.37 | C |
| ATOM | 1154 | CD1 | PHE | H | 146 | 22.879 | 38.763 | 17.884 | 1.00 | 20.87 | C |
| ATOM | 1155 | CE1 | PHE | H | 146 | 21.845 | 39.637 | 18.137 | 1.00 | 20.52 | C |
| ATOM | 1156 | CZ | PHE | H | 146 | 20.800 | 39.243 | 18.976 | 1.00 | 22.04 | C |
| ATOM | 1157 | CE2 | PHE | H | 146 | 20.812 | 37.963 | 19.566 | 1.00 | 22.31 | C |
| ATOM | 1158 | CD2 | PHE | H | 146 | 21.859 | 37.097 | 19.300 | 1.00 | 21.47 | C |
| ATOM | 1159 | C | PHE | H | 146 | 23.658 | 36.623 | 15.694 | 1.00 | 25.36 | C |
| ATOM | 1160 | O | PHE | H | 146 | 24.658 | 37.076 | 15.115 | 1.00 | 25.76 | O |
| ATOM | 1161 | N | PRO | H | 147 | 22.406 | 36.862 | 15.268 | 1.00 | 25.03 | N |
| ATOM | 1162 | CA | PRO | H | 147 | 21.178 | 36.249 | 15.768 | 1.00 | 25.44 | C |
| ATOM | 1163 | CB | PRO | H | 147 | 20.127 | 37.349 | 15.546 | 1.00 | 25.52 | C |
| ATOM | 1164 | CG | PRO | H | 147 | 20.693 | 38.215 | 14.408 | 1.00 | 25.34 | C |
| ATOM | 1165 | CD | PRO | H | 147 | 22.138 | 37.853 | 14.210 | 1.00 | 24.96 | C |
| ATOM | 1166 | C | PRO | H | 147 | 20.825 | 35.002 | 14.955 | 1.00 | 25.55 | C |
| ATOM | 1167 | O | PRO | H | 147 | 21.642 | 34.549 | 14.147 | 1.00 | 25.68 | O |
| ATOM | 1168 | N | GLU | H | 148 | 19.637 | 34.450 | 15.180 | 1.00 | 25.55 | N |
| ATOM | 1169 | CA | GLU | H | 148 | 19.102 | 33.381 | 14.326 | 1.00 | 26.22 | C |
| ATOM | 1170 | CB | GLU | H | 148 | 17.831 | 32.767 | 14.955 | 1.00 | 26.12 | C |
| ATOM | 1171 | CG | GLU | H | 148 | 18.073 | 31.796 | 16.119 | 1.00 | 27.28 | C |
| ATOM | 1172 | CD | GLU | H | 148 | 18.210 | 30.323 | 15.678 | 1.00 | 30.30 | C |
| ATOM | 1173 | OE1 | GLU | H | 148 | 19.206 | 29.959 | 14.989 | 1.00 | 29.18 | O |
| ATOM | 1174 | OE2 | GLU | H | 148 | 17.316 | 29.519 | 16.042 | 1.00 | 30.79 | O |
| ATOM | 1175 | C | GLU | H | 148 | 18.795 | 33.960 | 12.927 | 1.00 | 26.04 | C |
| ATOM | 1176 | O | GLU | H | 148 | 18.611 | 35.173 | 12.786 | 1.00 | 26.14 | O |
| ATOM | 1177 | N | PRO | H | 149 | 18.745 | 33.109 | 11.887 | 1.00 | 25.89 | N |
| ATOM | 1178 | CA | PRO | H | 149 | 19.018 | 31.678 | 11.861 | 1.00 | 26.11 | C |
| ATOM | 1179 | CB | PRO | H | 149 | 18.039 | 31.182 | 10.801 | 1.00 | 26.16 | C |
| ATOM | 1180 | CG | PRO | H | 149 | 18.071 | 32.304 | 9.774 | 1.00 | 25.87 | C |
| ATOM | 1181 | CD | PRO | H | 149 | 18.114 | 33.558 | 10.627 | 1.00 | 25.87 | C |
| ATOM | 1182 | C | PRO | H | 149 | 20.431 | 31.333 | 11.393 | 1.00 | 26.17 | C |
| ATOM | 1183 | O | PRO | H | 149 | 21.228 | 32.214 | 11.074 | 1.00 | 26.27 | O |
| ATOM | 1184 | N | VAL | H | 150 | 20.703 | 30.038 | 11.353 | 1.00 | 26.08 | N |
| ATOM | 1185 | CA | VAL | H | 150 | 21.852 | 29.482 | 10.668 | 1.00 | 26.25 | C |
| ATOM | 1186 | CB | VAL | H | 150 | 22.897 | 28.947 | 11.688 | 1.00 | 26.60 | C |
| ATOM | 1187 | CG1 | VAL | H | 150 | 22.460 | 27.619 | 12.296 | 1.00 | 25.07 | C |
| ATOM | 1188 | CG2 | VAL | H | 150 | 24.262 | 28.815 | 11.050 | 1.00 | 26.37 | C |
| ATOM | 1189 | C | VAL | H | 150 | 21.304 | 28.349 | 9.792 | 1.00 | 26.47 | C |
| ATOM | 1190 | O | VAL | H | 150 | 20.248 | 27.784 | 10.094 | 1.00 | 26.39 | O |
| ATOM | 1191 | N | THR | H | 151 | 21.982 | 28.040 | 8.695 | 1.00 | 26.58 | N |
| ATOM | 1192 | CA | THR | H | 151 | 21.598 | 26.873 | 7.903 | 1.00 | 27.24 | C |
| ATOM | 1193 | CB | THR | H | 151 | 21.132 | 27.261 | 6.467 | 1.00 | 27.46 | C |
| ATOM | 1194 | OG1 | THR | H | 151 | 22.199 | 27.917 | 5.773 | 1.00 | 29.23 | O |
| ATOM | 1195 | CG2 | THR | H | 151 | 19.932 | 28.202 | 6.510 | 1.00 | 27.59 | C |
| ATOM | 1196 | C | THR | H | 151 | 22.754 | 25.857 | 7.871 | 1.00 | 27.06 | C |
| ATOM | 1197 | O | THR | H | 151 | 23.931 | 26.239 | 7.869 | 1.00 | 27.07 | O |
| ATOM | 1198 | N | VAL | H | 152 | 22.416 | 24.568 | 7.874 | 1.00 | 26.73 | N |
| ATOM | 1199 | CA | VAL | H | 152 | 23.423 | 23.501 | 7.902 | 1.00 | 26.32 | C |
| ATOM | 1200 | CB | VAL | H | 152 | 23.544 | 22.818 | 9.319 | 1.00 | 26.38 | C |
| ATOM | 1201 | CG1 | VAL | H | 152 | 24.692 | 21.821 | 9.342 | 1.00 | 25.31 | C |
| ATOM | 1202 | CG2 | VAL | H | 152 | 23.739 | 23.854 | 10.421 | 1.00 | 25.74 | C |
| ATOM | 1203 | C | VAL | H | 152 | 23.097 | 22.432 | 6.873 | 1.00 | 26.41 | C |
| ATOM | 1204 | O | VAL | H | 152 | 21.986 | 21.912 | 6.844 | 1.00 | 26.89 | O |
| ATOM | 1205 | N | SER | H | 153 | 24.065 | 22.098 | 6.033 | 1.00 | 26.44 | N |
| ATOM | 1206 | CA | SER | H | 153 | 23.902 | 20.979 | 5.127 | 1.00 | 26.63 | C |
| ATOM | 1207 | CB | SER | H | 153 | 23.740 | 21.465 | 3.692 | 1.00 | 26.84 | C |
| ATOM | 1208 | OG | SER | H | 153 | 24.883 | 22.211 | 3.292 | 1.00 | 28.69 | O |
| ATOM | 1209 | C | SER | H | 153 | 25.114 | 20.080 | 5.247 | 1.00 | 26.42 | C |
| ATOM | 1210 | O | SER | H | 153 | 26.119 | 20.469 | 5.839 | 1.00 | 26.09 | O |
| ATOM | 1211 | N | TRP | H | 154 | 24.995 | 18.880 | 4.687 | 1.00 | 26.31 | N |
| ATOM | 1212 | CA | TRP | H | 154 | 26.063 | 17.900 | 4.670 | 1.00 | 26.46 | C |
| ATOM | 1213 | CB | TRP | H | 154 | 25.646 | 16.648 | 5.449 | 1.00 | 26.37 | C |
| ATOM | 1214 | CG | TRP | H | 154 | 25.613 | 16.892 | 6.927 | 1.00 | 26.76 | C |
| ATOM | 1215 | CD1 | TRP | H | 154 | 24.533 | 17.277 | 7.673 | 1.00 | 27.04 | C |
| ATOM | 1216 | NE1 | TRP | H | 154 | 24.895 | 17.427 | 8.995 | 1.00 | 27.13 | N |
| ATOM | 1217 | CE2 | TRP | H | 154 | 26.231 | 17.148 | 9.123 | 1.00 | 27.52 | C |
| ATOM | 1218 | CD2 | TRP | H | 154 | 26.719 | 16.808 | 7.837 | 1.00 | 27.27 | C |
| ATOM | 1219 | CE3 | TRP | H | 154 | 28.075 | 16.462 | 7.695 | 1.00 | 26.91 | C |
| ATOM | 1220 | CZ3 | TRP | H | 154 | 28.891 | 16.475 | 8.822 | 1.00 | 25.54 | C |
| ATOM | 1221 | CH2 | TRP | H | 154 | 28.375 | 16.818 | 10.090 | 1.00 | 25.68 | C |
| ATOM | 1222 | CZ2 | TRP | H | 154 | 27.053 | 17.152 | 10.263 | 1.00 | 26.64 | C |
| ATOM | 1223 | C | TRP | H | 154 | 26.464 | 17.556 | 3.232 | 1.00 | 26.76 | C |
| ATOM | 1224 | O | TRP | H | 154 | 25.611 | 17.210 | 2.398 | 1.00 | 26.80 | O |
| ATOM | 1225 | N | ASN | H | 155 | 27.769 | 17.641 | 2.953 | 1.00 | 27.12 | N |
| ATOM | 1226 | CA | ASN | H | 155 | 28.320 | 17.395 | 1.616 | 1.00 | 27.15 | C |
| ATOM | 1227 | CB | ASN | H | 155 | 28.256 | 15.900 | 1.244 | 1.00 | 26.95 | C |
| ATOM | 1228 | CG | ASN | H | 155 | 29.121 | 15.024 | 2.153 | 1.00 | 26.78 | C |
| ATOM | 1229 | OD1 | ASN | H | 155 | 30.080 | 15.492 | 2.778 | 1.00 | 26.54 | O |
| ATOM | 1230 | ND2 | ASN | H | 155 | 28.792 | 13.742 | 2.213 | 1.00 | 25.33 | N |
| ATOM | 1231 | C | ASN | H | 155 | 27.596 | 18.253 | 0.587 | 1.00 | 27.51 | C |
| ATOM | 1232 | O | ASN | H | 155 | 27.098 | 17.757 | −0.427 | 1.00 | 27.78 | O |
| ATOM | 1233 | N | SER | H | 156 | 27.504 | 19.544 | 0.887 | 1.00 | 27.69 | N |
| ATOM | 1234 | CA | SER | H | 156 | 26.871 | 20.519 | −0.007 | 1.00 | 27.76 | C |
| ATOM | 1235 | CB | SER | H | 156 | 27.765 | 20.769 | −1.224 | 1.00 | 27.77 | C |
| ATOM | 1236 | OG | SER | H | 156 | 28.957 | 21.415 | −0.814 | 1.00 | 28.43 | O |
| ATOM | 1237 | C | SER | H | 156 | 25.437 | 20.167 | −0.420 | 1.00 | 27.56 | C |
| ATOM | 1238 | O | SER | H | 156 | 25.012 | 20.471 | −1.534 | 1.00 | 28.14 | O |
| ATOM | 1239 | N | GLY | H | 157 | 24.703 | 19.530 | 0.492 | 1.00 | 27.25 | N |
| ATOM | 1240 | CA | GLY | H | 157 | 23.299 | 19.203 | 0.294 | 1.00 | 26.09 | C |
| ATOM | 1241 | C | GLY | H | 157 | 23.072 | 17.824 | −0.281 | 1.00 | 25.97 | C |
| ATOM | 1242 | O | GLY | H | 157 | 21.929 | 17.428 | −0.469 | 1.00 | 25.95 | O |
| ATOM | 1243 | N | ALA | H | 158 | 24.153 | 17.090 | −0.562 | 1.00 | 25.87 | N |
| ATOM | 1244 | CA | ALA | H | 158 | 24.072 | 15.755 | −1.189 | 1.00 | 25.22 | C |
| ATOM | 1245 | CB | ALA | H | 158 | 25.375 | 15.414 | −1.898 | 1.00 | 25.02 | C |
| ATOM | 1246 | C | ALA | H | 158 | 23.727 | 14.658 | −0.193 | 1.00 | 25.39 | C |
| ATOM | 1247 | O | ALA | H | 158 | 23.497 | 13.492 | −0.578 | 1.00 | 25.62 | O |
| ATOM | 1248 | N | LEU | H | 159 | 23.716 | 15.018 | 1.092 | 1.00 | 24.93 | N |
| ATOM | 1249 | CA | LEU | H | 159 | 23.373 | 14.071 | 2.149 | 1.00 | 24.15 | C |
| ATOM | 1250 | CB | LEU | H | 159 | 24.623 | 13.738 | 2.946 | 1.00 | 23.93 | C |
| ATOM | 1251 | CG | LEU | H | 159 | 24.482 | 12.815 | 4.149 | 1.00 | 24.58 | C |
| ATOM | 1252 | CD1 | LEU | H | 159 | 23.893 | 11.451 | 3.760 | 1.00 | 24.31 | C |
| ATOM | 1253 | CD2 | LEU | H | 159 | 25.854 | 12.676 | 4.842 | 1.00 | 23.67 | C |
| ATOM | 1254 | C | LEU | H | 159 | 22.254 | 14.618 | 3.038 | 1.00 | 23.39 | C |
| ATOM | 1255 | O | LEU | H | 159 | 22.385 | 15.695 | 3.592 | 1.00 | 23.59 | O |
| ATOM | 1256 | N | THR | H | 160 | 21.145 | 13.890 | 3.142 | 1.00 | 22.78 | N |
| ATOM | 1257 | CA | THR | H | 160 | 20.007 | 14.337 | 3.949 | 1.00 | 22.17 | C |
| ATOM | 1258 | CB | THR | H | 160 | 18.858 | 14.924 | 3.100 | 1.00 | 22.09 | C |
| ATOM | 1259 | OG1 | THR | H | 160 | 18.288 | 13.898 | 2.272 | 1.00 | 21.12 | O |
| ATOM | 1260 | CG2 | THR | H | 160 | 19.322 | 16.104 | 2.260 | 1.00 | 20.42 | C |
| ATOM | 1261 | C | THR | H | 160 | 19.413 | 13.218 | 4.807 | 1.00 | 22.69 | C |
| ATOM | 1262 | O | THR | H | 160 | 18.839 | 13.491 | 5.873 | 1.00 | 22.41 | O |
| ATOM | 1263 | N | SER | H | 161 | 19.529 | 11.975 | 4.324 | 1.00 | 22.66 | N |
| ATOM | 1264 | CA | SER | H | 161 | 19.065 | 10.788 | 5.061 | 1.00 | 22.84 | C |
| ATOM | 1265 | CB | SER | H | 161 | 19.157 | 9.527 | 4.197 | 1.00 | 22.85 | C |
| ATOM | 1266 | OG | SER | H | 161 | 18.519 | 9.733 | 2.945 | 1.00 | 23.44 | O |
| ATOM | 1267 | C | SER | H | 161 | 19.872 | 10.593 | 6.330 | 1.00 | 22.67 | C |
| ATOM | 1268 | O | SER | H | 161 | 21.097 | 10.696 | 6.309 | 1.00 | 22.90 | O |
| ATOM | 1269 | N | GLY | H | 162 | 19.184 | 10.319 | 7.434 | 1.00 | 22.44 | N |
| ATOM | 1270 | CA | GLY | H | 162 | 19.841 | 10.198 | 8.727 | 1.00 | 22.07 | C |
| ATOM | 1271 | C | GLY | H | 162 | 20.328 | 11.493 | 9.373 | 1.00 | 21.97 | C |
| ATOM | 1272 | O | GLY | H | 162 | 20.886 | 11.444 | 10.470 | 1.00 | 22.30 | O |
| ATOM | 1273 | N | VAL | H | 163 | 20.106 | 12.643 | 8.727 | 1.00 | 21.42 | N |
| ATOM | 1274 | CA | VAL | H | 163 | 20.503 | 13.945 | 9.289 | 1.00 | 20.76 | C |
| ATOM | 1275 | CB | VAL | H | 163 | 20.728 | 15.000 | 8.197 | 1.00 | 20.72 | C |
| ATOM | 1276 | CG1 | VAL | H | 163 | 20.920 | 16.380 | 8.804 | 1.00 | 20.08 | C |
| ATOM | 1277 | CG2 | VAL | H | 163 | 21.924 | 14.626 | 7.311 | 1.00 | 20.76 | C |
| ATOM | 1278 | C | VAL | H | 163 | 19.471 | 14.484 | 10.289 | 1.00 | 20.89 | C |
| ATOM | 1279 | O | VAL | H | 163 | 18.268 | 14.430 | 10.044 | 1.00 | 20.94 | O |
| ATOM | 1280 | N | HIS | H | 164 | 19.956 | 14.973 | 11.426 | 1.00 | 20.24 | N |
| ATOM | 1281 | CA | HIS | H | 164 | 19.128 | 15.672 | 12.390 | 1.00 | 19.94 | C |
| ATOM | 1282 | CB | HIS | H | 164 | 18.883 | 14.833 | 13.647 | 1.00 | 19.33 | C |
| ATOM | 1283 | CG | HIS | H | 164 | 18.076 | 13.590 | 13.407 | 1.00 | 19.65 | C |
| ATOM | 1284 | ND1 | HIS | H | 164 | 16.801 | 13.611 | 12.879 | 1.00 | 19.60 | N |
| ATOM | 1285 | CE1 | HIS | H | 164 | 16.338 | 12.378 | 12.799 | 1.00 | 17.00 | C |
| ATOM | 1286 | NE2 | HIS | H | 164 | 17.259 | 11.559 | 13.269 | 1.00 | 16.60 | N |
| ATOM | 1287 | CD2 | HIS | H | 164 | 18.356 | 12.289 | 13.652 | 1.00 | 18.16 | C |
| ATOM | 1288 | C | HIS | H | 164 | 19.837 | 16.966 | 12.756 | 1.00 | 19.99 | C |
| ATOM | 1289 | O | HIS | H | 164 | 20.925 | 16.952 | 13.334 | 1.00 | 20.22 | O |
| ATOM | 1290 | N | THR | H | 165 | 19.226 | 18.082 | 12.389 | 1.00 | 19.81 | N |
| ATOM | 1291 | CA | THR | H | 165 | 19.742 | 19.390 | 12.741 | 1.00 | 19.60 | C |
| ATOM | 1292 | CB | THR | H | 165 | 19.862 | 20.300 | 11.488 | 1.00 | 19.67 | C |
| ATOM | 1293 | OG1 | THR | H | 165 | 20.870 | 19.766 | 10.620 | 1.00 | 19.45 | O |
| ATOM | 1294 | CG2 | THR | H | 165 | 20.211 | 21.751 | 11.863 | 1.00 | 18.59 | C |
| ATOM | 1295 | C | THR | H | 165 | 18.815 | 19.975 | 13.810 | 1.00 | 19.66 | C |
| ATOM | 1296 | O | THR | H | 165 | 17.669 | 20.358 | 13.537 | 1.00 | 19.06 | O |
| ATOM | 1297 | N | PHE | H | 166 | 19.338 | 20.021 | 15.028 | 1.00 | 19.55 | N |
| ATOM | 1298 | CA | PHE | H | 166 | 18.566 | 20.352 | 16.201 | 1.00 | 19.90 | C |
| ATOM | 1299 | CB | PHE | H | 166 | 19.348 | 19.935 | 17.443 | 1.00 | 19.75 | C |
| ATOM | 1300 | CG | PHE | H | 166 | 19.428 | 18.454 | 17.603 | 1.00 | 19.58 | C |
| ATOM | 1301 | CD1 | PHE | H | 166 | 18.396 | 17.758 | 18.217 | 1.00 | 18.81 | C |
| ATOM | 1302 | CE1 | PHE | H | 166 | 18.446 | 16.380 | 18.329 | 1.00 | 18.25 | C |
| ATOM | 1303 | CZ | PHE | H | 166 | 19.525 | 15.686 | 17.818 | 1.00 | 16.80 | C |
| ATOM | 1304 | CE2 | PHE | H | 166 | 20.549 | 16.367 | 17.197 | 1.00 | 17.00 | C |
| ATOM | 1305 | CD2 | PHE | H | 166 | 20.501 | 17.742 | 17.083 | 1.00 | 18.08 | C |
| ATOM | 1306 | C | PHE | H | 166 | 18.179 | 21.818 | 16.265 | 1.00 | 20.45 | C |
| ATOM | 1307 | O | PHE | H | 166 | 18.899 | 22.672 | 15.737 | 1.00 | 21.44 | O |
| ATOM | 1308 | N | PRO | H | 167 | 17.035 | 22.124 | 16.890 | 1.00 | 20.42 | N |
| ATOM | 1309 | CA | PRO | H | 167 | 16.755 | 23.544 | 17.110 | 1.00 | 20.69 | C |
| ATOM | 1310 | CB | PRO | H | 167 | 15.388 | 23.546 | 17.817 | 1.00 | 20.33 | C |
| ATOM | 1311 | CG | PRO | H | 167 | 14.788 | 22.189 | 17.555 | 1.00 | 21.19 | C |
| ATOM | 1312 | CD | PRO | H | 167 | 15.957 | 21.241 | 17.378 | 1.00 | 20.67 | C |
| ATOM | 1313 | C | PRO | H | 167 | 17.824 | 24.148 | 18.033 | 1.00 | 21.09 | C |
| ATOM | 1314 | O | PRO | H | 167 | 18.353 | 23.454 | 18.913 | 1.00 | 20.87 | O |
| ATOM | 1315 | N | ALA | H | 168 | 18.125 | 25.426 | 17.825 | 1.00 | 21.39 | N |
| ATOM | 1316 | CA | ALA | H | 168 | 19.102 | 26.149 | 18.629 | 1.00 | 22.06 | C |
| ATOM | 1317 | CB | ALA | H | 168 | 19.364 | 27.505 | 18.015 | 1.00 | 22.16 | C |
| ATOM | 1318 | C | ALA | H | 168 | 18.641 | 26.318 | 20.066 | 1.00 | 22.47 | C |
| ATOM | 1319 | O | ALA | H | 168 | 17.443 | 26.322 | 20.329 | 1.00 | 22.62 | O |
| ATOM | 1320 | N | VAL | H | 169 | 19.596 | 26.441 | 20.990 | 1.00 | 22.85 | N |
| ATOM | 1321 | CA | VAL | H | 169 | 19.313 | 26.863 | 22.371 | 1.00 | 23.14 | C |
| ATOM | 1322 | CB | VAL | H | 169 | 19.676 | 25.783 | 23.424 | 1.00 | 23.27 | C |
| ATOM | 1323 | CG1 | VAL | H | 169 | 18.953 | 24.476 | 23.126 | 1.00 | 23.13 | C |
| ATOM | 1324 | CG2 | VAL | H | 169 | 21.209 | 25.576 | 23.526 | 1.00 | 23.09 | C |
| ATOM | 1325 | C | VAL | H | 169 | 20.050 | 28.168 | 22.695 | 1.00 | 23.48 | C |
| ATOM | 1326 | O | VAL | H | 169 | 21.203 | 28.353 | 22.314 | 1.00 | 23.65 | O |
| ATOM | 1327 | N | LEU | H | 170 | 19.368 | 29.078 | 23.376 | 1.00 | 23.86 | N |
| ATOM | 1328 | CA | LEU | H | 170 | 19.957 | 30.352 | 23.748 | 1.00 | 23.96 | C |
| ATOM | 1329 | CB | LEU | H | 170 | 18.868 | 31.404 | 23.987 | 1.00 | 23.59 | C |
| ATOM | 1330 | CG | LEU | H | 170 | 19.363 | 32.779 | 24.474 | 1.00 | 24.30 | C |
| ATOM | 1331 | CD1 | LEU | H | 170 | 20.403 | 33.409 | 23.538 | 1.00 | 22.43 | C |
| ATOM | 1332 | CD2 | LEU | H | 170 | 18.190 | 33.738 | 24.670 | 1.00 | 24.33 | C |
| ATOM | 1333 | C | LEU | H | 170 | 20.763 | 30.149 | 25.009 | 1.00 | 24.15 | C |
| ATOM | 1334 | O | LEU | H | 170 | 20.222 | 29.709 | 26.025 | 1.00 | 24.78 | O |
| ATOM | 1335 | N | GLN | H | 171 | 22.048 | 30.466 | 24.951 | 1.00 | 24.24 | N |
| ATOM | 1336 | CA | GLN | H | 171 | 22.912 | 30.327 | 26.122 | 1.00 | 24.89 | C |
| ATOM | 1337 | CB | GLN | H | 171 | 24.352 | 30.023 | 25.688 | 1.00 | 24.74 | C |
| ATOM | 1338 | CG | GLN | H | 171 | 24.474 | 28.789 | 24.783 | 1.00 | 25.48 | C |
| ATOM | 1339 | CD | GLN | H | 171 | 25.760 | 28.767 | 23.968 | 1.00 | 27.46 | C |
| ATOM | 1340 | OE1 | GLN | H | 171 | 26.666 | 27.959 | 24.227 | 1.00 | 25.30 | O |
| ATOM | 1341 | NE2 | GLN | H | 171 | 25.844 | 29.652 | 22.963 | 1.00 | 27.64 | N |
| ATOM | 1342 | C | GLN | H | 171 | 22.839 | 31.605 | 26.961 | 1.00 | 25.19 | C |
| ATOM | 1343 | O | GLN | H | 171 | 22.428 | 32.651 | 26.461 | 1.00 | 25.50 | O |
| ATOM | 1344 | N | SER | H | 172 | 23.239 | 31.522 | 28.228 | 1.00 | 25.56 | N |
| ATOM | 1345 | CA | SER | H | 172 | 23.292 | 32.697 | 29.107 | 1.00 | 25.88 | C |
| ATOM | 1346 | CB | SER | H | 172 | 23.711 | 32.310 | 30.525 | 1.00 | 25.70 | C |
| ATOM | 1347 | OG | SER | H | 172 | 24.826 | 31.449 | 30.518 | 1.00 | 25.56 | O |
| ATOM | 1348 | C | SER | H | 172 | 24.179 | 33.838 | 28.578 | 1.00 | 26.28 | C |
| ATOM | 1349 | O | SER | H | 172 | 23.986 | 35.003 | 28.954 | 1.00 | 26.43 | O |
| ATOM | 1350 | N | SER | H | 173 | 25.131 | 33.508 | 27.703 | 1.00 | 26.12 | N |
| ATOM | 1351 | CA | SER | H | 173 | 25.960 | 34.522 | 27.049 | 1.00 | 26.24 | C |
| ATOM | 1352 | CB | SER | H | 173 | 27.103 | 33.853 | 26.291 | 1.00 | 26.16 | C |
| ATOM | 1353 | OG | SER | H | 173 | 26.607 | 32.999 | 25.271 | 1.00 | 27.18 | O |
| ATOM | 1354 | C | SER | H | 173 | 25.169 | 35.427 | 26.089 | 1.00 | 26.18 | C |
| ATOM | 1355 | O | SER | H | 173 | 25.689 | 36.445 | 25.617 | 1.00 | 26.23 | O |
| ATOM | 1356 | N | GLY | H | 174 | 23.917 | 35.055 | 25.812 | 1.00 | 25.75 | N |
| ATOM | 1357 | CA | GLY | H | 174 | 23.105 | 35.729 | 24.808 | 1.00 | 25.16 | C |
| ATOM | 1358 | C | GLY | H | 174 | 23.377 | 35.199 | 23.409 | 1.00 | 24.97 | C |
| ATOM | 1359 | O | GLY | H | 174 | 22.832 | 35.690 | 22.432 | 1.00 | 25.45 | O |
| ATOM | 1360 | N | LEU | H | 175 | 24.223 | 34.191 | 23.304 | 1.00 | 24.53 | N |
| ATOM | 1361 | CA | LEU | H | 175 | 24.514 | 33.596 | 22.014 | 1.00 | 24.23 | C |
| ATOM | 1362 | CB | LEU | H | 175 | 26.021 | 33.394 | 21.847 | 1.00 | 24.10 | C |
| ATOM | 1363 | CG | LEU | H | 175 | 26.919 | 34.596 | 22.137 | 1.00 | 23.42 | C |
| ATOM | 1364 | CD1 | LEU | H | 175 | 28.366 | 34.179 | 21.991 | 1.00 | 22.88 | C |
| ATOM | 1365 | CD2 | LEU | H | 175 | 26.585 | 35.818 | 21.255 | 1.00 | 19.92 | C |
| ATOM | 1366 | C | LEU | H | 175 | 23.791 | 32.263 | 21.856 | 1.00 | 24.34 | C |
| ATOM | 1367 | O | LEU | H | 175 | 23.440 | 31.610 | 22.853 | 1.00 | 24.15 | O |
| ATOM | 1368 | N | TYR | H | 176 | 23.588 | 31.865 | 20.597 | 1.00 | 24.17 | N |
| ATOM | 1369 | CA | TYR | H | 176 | 22.889 | 30.629 | 20.264 | 1.00 | 23.96 | C |
| ATOM | 1370 | CB | TYR | H | 176 | 22.005 | 30.831 | 19.039 | 1.00 | 23.77 | C |
| ATOM | 1371 | CG | TYR | H | 176 | 20.811 | 31.711 | 19.287 | 1.00 | 22.91 | C |
| ATOM | 1372 | CD1 | TYR | H | 176 | 19.612 | 31.179 | 19.778 | 1.00 | 21.45 | C |
| ATOM | 1373 | CE1 | TYR | H | 176 | 18.516 | 31.990 | 19.996 | 1.00 | 21.16 | C |
| ATOM | 1374 | CZ | TYR | H | 176 | 18.613 | 33.346 | 19.733 | 1.00 | 21.00 | C |
| ATOM | 1375 | OH | TYR | H | 176 | 17.541 | 34.164 | 19.939 | 1.00 | 22.41 | O |
| ATOM | 1376 | CE2 | TYR | H | 176 | 19.780 | 33.895 | 19.257 | 1.00 | 21.59 | C |
| ATOM | 1377 | CD2 | TYR | H | 176 | 20.873 | 33.079 | 19.028 | 1.00 | 21.98 | C |
| ATOM | 1378 | C | TYR | H | 176 | 23.853 | 29.501 | 19.984 | 1.00 | 24.19 | C |
| ATOM | 1379 | O | TYR | H | 176 | 25.025 | 29.733 | 19.662 | 1.00 | 24.68 | O |
| ATOM | 1380 | N | SER | H | 177 | 23.347 | 28.277 | 20.087 | 1.00 | 24.16 | N |
| ATOM | 1381 | CA | SER | H | 177 | 24.167 | 27.096 | 19.925 | 1.00 | 24.38 | C |
| ATOM | 1382 | CB | SER | H | 177 | 24.855 | 26.765 | 21.249 | 1.00 | 24.50 | C |
| ATOM | 1383 | OG | SER | H | 177 | 25.818 | 25.743 | 21.090 | 1.00 | 25.89 | O |
| ATOM | 1384 | C | SER | H | 177 | 23.309 | 25.930 | 19.485 | 1.00 | 24.55 | C |
| ATOM | 1385 | O | SER | H | 177 | 22.288 | 25.636 | 20.102 | 1.00 | 25.28 | O |
| ATOM | 1386 | N | LEU | H | 178 | 23.713 | 25.263 | 18.412 | 1.00 | 24.63 | N |
| ATOM | 1387 | CA | LEU | H | 178 | 23.020 | 24.053 | 17.966 | 1.00 | 24.25 | C |
| ATOM | 1388 | CB | LEU | H | 178 | 21.968 | 24.351 | 16.880 | 1.00 | 24.21 | C |
| ATOM | 1389 | CG | LEU | H | 178 | 22.324 | 24.788 | 15.448 | 1.00 | 24.41 | C |
| ATOM | 1390 | CD1 | LEU | H | 178 | 22.917 | 23.655 | 14.591 | 1.00 | 23.72 | C |
| ATOM | 1391 | CD2 | LEU | H | 178 | 21.078 | 25.340 | 14.773 | 1.00 | 23.85 | C |
| ATOM | 1392 | C | LEU | H | 178 | 24.013 | 23.033 | 17.465 | 1.00 | 23.93 | C |
| ATOM | 1393 | O | LEU | H | 178 | 25.140 | 23.373 | 17.131 | 1.00 | 23.87 | O |
| ATOM | 1394 | N | SER | H | 179 | 23.571 | 21.784 | 17.434 | 1.00 | 23.73 | N |
| ATOM | 1395 | CA | SER | H | 179 | 24.298 | 20.700 | 16.823 | 1.00 | 23.65 | C |
| ATOM | 1396 | CB | SER | H | 179 | 24.425 | 19.539 | 17.802 | 1.00 | 23.99 | C |
| ATOM | 1397 | OG | SER | H | 179 | 25.664 | 19.623 | 18.491 | 1.00 | 26.54 | O |
| ATOM | 1398 | C | SER | H | 179 | 23.602 | 20.218 | 15.568 | 1.00 | 22.97 | C |
| ATOM | 1399 | O | SER | H | 179 | 22.377 | 20.299 | 15.452 | 1.00 | 22.50 | O |
| ATOM | 1400 | N | SER | H | 180 | 24.391 | 19.719 | 14.622 | 1.00 | 22.46 | N |
| ATOM | 1401 | CA | SER | H | 180 | 23.825 | 18.969 | 13.515 | 1.00 | 22.05 | C |
| ATOM | 1402 | CB | SER | H | 180 | 23.943 | 19.724 | 12.192 | 1.00 | 21.67 | C |
| ATOM | 1403 | OG | SER | H | 180 | 23.359 | 18.963 | 11.147 | 1.00 | 20.32 | O |
| ATOM | 1404 | C | SER | H | 180 | 24.524 | 17.635 | 13.443 | 1.00 | 22.21 | C |
| ATOM | 1405 | O | SER | H | 180 | 25.749 | 17.572 | 13.385 | 1.00 | 22.31 | O |
| ATOM | 1406 | N | VAL | H | 181 | 23.738 | 16.570 | 13.452 | 1.00 | 22.52 | N |
| ATOM | 1407 | CA | VAL | H | 181 | 24.282 | 15.223 | 13.455 | 1.00 | 23.06 | C |
| ATOM | 1408 | CB | VAL | H | 181 | 23.992 | 14.494 | 14.786 | 1.00 | 23.08 | C |
| ATOM | 1409 | CG1 | VAL | H | 181 | 24.547 | 15.284 | 15.969 | 1.00 | 22.03 | C |
| ATOM | 1410 | CG2 | VAL | H | 181 | 22.497 | 14.235 | 14.940 | 1.00 | 22.32 | C |
| ATOM | 1411 | C | VAL | H | 181 | 23.728 | 14.381 | 12.313 | 1.00 | 24.04 | C |
| ATOM | 1412 | O | VAL | H | 181 | 22.716 | 14.754 | 11.689 | 1.00 | 24.04 | O |
| ATOM | 1413 | N | VAL | H | 182 | 24.389 | 13.240 | 12.071 | 1.00 | 24.64 | N |
| ATOM | 1414 | CA | VAL | H | 182 | 24.033 | 12.293 | 11.009 | 1.00 | 25.06 | C |
| ATOM | 1415 | CB | VAL | H | 182 | 24.509 | 12.792 | 9.599 | 1.00 | 24.95 | C |
| ATOM | 1416 | CG1 | VAL | H | 182 | 25.986 | 13.100 | 9.583 | 1.00 | 24.53 | C |
| ATOM | 1417 | CG2 | VAL | H | 182 | 24.150 | 11.801 | 8.487 | 1.00 | 24.39 | C |
| ATOM | 1418 | C | VAL | H | 182 | 24.568 | 10.896 | 11.339 | 1.00 | 26.12 | C |
| ATOM | 1419 | O | VAL | H | 182 | 25.687 | 10.744 | 11.820 | 1.00 | 25.93 | O |
| ATOM | 1420 | N | THR | H | 183 | 23.746 | 9.878 | 11.116 | 1.00 | 27.65 | N |
| ATOM | 1421 | CA | THR | H | 183 | 24.167 | 8.504 | 11.320 | 1.00 | 29.26 | C |
| ATOM | 1422 | CB | THR | H | 183 | 23.092 | 7.641 | 12.016 | 1.00 | 29.09 | C |
| ATOM | 1423 | OG1 | THR | H | 183 | 21.908 | 7.586 | 11.205 | 1.00 | 29.55 | O |
| ATOM | 1424 | CG2 | THR | H | 183 | 22.760 | 8.184 | 13.392 | 1.00 | 29.07 | C |
| ATOM | 1425 | C | THR | H | 183 | 24.478 | 7.882 | 9.979 | 1.00 | 30.58 | C |
| ATOM | 1426 | O | THR | H | 183 | 23.726 | 8.046 | 9.030 | 1.00 | 30.70 | O |
| ATOM | 1427 | N | VAL | H | 184 | 25.588 | 7.158 | 9.913 | 1.00 | 32.43 | N |
| ATOM | 1428 | CA | VAL | H | 184 | 26.029 | 6.512 | 8.679 | 1.00 | 33.98 | C |
| ATOM | 1429 | CB | VAL | H | 184 | 27.100 | 7.357 | 7.950 | 1.00 | 33.95 | C |
| ATOM | 1430 | CG1 | VAL | H | 184 | 26.525 | 8.677 | 7.481 | 1.00 | 32.53 | C |
| ATOM | 1431 | CG2 | VAL | H | 184 | 28.321 | 7.590 | 8.842 | 1.00 | 34.24 | C |
| ATOM | 1432 | C | VAL | H | 184 | 26.568 | 5.107 | 8.982 | 1.00 | 35.52 | C |
| ATOM | 1433 | O | VAL | H | 184 | 26.889 | 4.807 | 10.134 | 1.00 | 35.63 | O |
| ATOM | 1434 | N | PRO | H | 185 | 26.638 | 4.227 | 7.962 | 1.00 | 37.03 | N |
| ATOM | 1435 | CA | PRO | H | 185 | 27.271 | 2.916 | 8.168 | 1.00 | 37.79 | C |
| ATOM | 1436 | CB | PRO | H | 185 | 27.178 | 2.265 | 6.786 | 1.00 | 37.79 | C |
| ATOM | 1437 | CG | PRO | H | 185 | 25.972 | 2.885 | 6.165 | 1.00 | 37.72 | C |
| ATOM | 1438 | CD | PRO | H | 185 | 26.010 | 4.325 | 6.628 | 1.00 | 37.34 | C |
| ATOM | 1439 | C | PRO | H | 185 | 28.733 | 3.075 | 8.573 | 1.00 | 38.71 | C |
| ATOM | 1440 | O | PRO | H | 185 | 29.452 | 3.883 | 7.972 | 1.00 | 38.82 | O |
| ATOM | 1441 | N | SER | H | 186 | 29.169 | 2.325 | 9.584 | 1.00 | 39.75 | N |
| ATOM | 1442 | CA | SER | H | 186 | 30.539 | 2.473 | 10.084 | 1.00 | 41.05 | C |
| ATOM | 1443 | CB | SER | H | 186 | 30.721 | 1.822 | 11.459 | 1.00 | 41.25 | C |
| ATOM | 1444 | OG | SER | H | 186 | 30.427 | 0.437 | 11.431 | 1.00 | 42.83 | O |
| ATOM | 1445 | C | SER | H | 186 | 31.588 | 1.976 | 9.083 | 1.00 | 41.54 | C |
| ATOM | 1446 | O | SER | H | 186 | 32.746 | 2.417 | 9.120 | 1.00 | 41.52 | O |
| ATOM | 1447 | N | SER | H | 187 | 31.168 | 1.094 | 8.174 | 1.00 | 42.13 | N |
| ATOM | 1448 | CA | SER | H | 187 | 32.040 | 0.619 | 7.097 | 1.00 | 42.86 | C |
| ATOM | 1449 | CB | SER | H | 187 | 31.419 | −0.577 | 6.355 | 1.00 | 42.95 | C |
| ATOM | 1450 | OG | SER | H | 187 | 30.151 | −0.275 | 5.799 | 1.00 | 43.22 | O |
| ATOM | 1451 | C | SER | H | 187 | 32.451 | 1.728 | 6.115 | 1.00 | 43.38 | C |
| ATOM | 1452 | O | SER | H | 187 | 33.508 | 1.636 | 5.490 | 1.00 | 43.55 | O |
| ATOM | 1453 | N | SER | H | 188 | 31.635 | 2.778 | 6.003 | 1.00 | 43.86 | N |
| ATOM | 1454 | CA | SER | H | 188 | 31.919 | 3.896 | 5.090 | 1.00 | 44.23 | C |
| ATOM | 1455 | CB | SER | H | 188 | 30.616 | 4.574 | 4.645 | 1.00 | 44.38 | C |
| ATOM | 1456 | OG | SER | H | 188 | 30.080 | 5.399 | 5.673 | 1.00 | 44.92 | O |
| ATOM | 1457 | C | SER | H | 188 | 32.881 | 4.947 | 5.664 | 1.00 | 44.51 | C |
| ATOM | 1458 | O | SER | H | 188 | 33.330 | 5.841 | 4.943 | 1.00 | 44.44 | O |
| ATOM | 1459 | N | LEU | H | 189 | 33.201 | 4.840 | 6.951 | 1.00 | 44.95 | N |
| ATOM | 1460 | CA | LEU | H | 189 | 34.015 | 5.860 | 7.625 | 1.00 | 45.32 | C |
| ATOM | 1461 | CB | LEU | H | 189 | 34.122 | 5.569 | 9.126 | 1.00 | 45.26 | C |
| ATOM | 1462 | CG | LEU | H | 189 | 32.891 | 5.789 | 10.023 | 1.00 | 44.83 | C |
| ATOM | 1463 | CD1 | LEU | H | 189 | 33.232 | 5.519 | 11.486 | 1.00 | 43.77 | C |
| ATOM | 1464 | CD2 | LEU | H | 189 | 32.323 | 7.188 | 9.869 | 1.00 | 43.39 | C |
| ATOM | 1465 | C | LEU | H | 189 | 35.411 | 6.104 | 7.025 | 1.00 | 45.81 | C |
| ATOM | 1466 | O | LEU | H | 189 | 35.967 | 7.194 | 7.188 | 1.00 | 46.29 | O |
| ATOM | 1467 | N | GLY | H | 190 | 35.972 | 5.103 | 6.343 | 1.00 | 45.85 | N |
| ATOM | 1468 | CA | GLY | H | 190 | 37.273 | 5.246 | 5.676 | 1.00 | 45.72 | C |
| ATOM | 1469 | C | GLY | H | 190 | 37.170 | 5.743 | 4.239 | 1.00 | 45.78 | C |
| ATOM | 1470 | O | GLY | H | 190 | 38.106 | 6.367 | 3.719 | 1.00 | 45.94 | O |
| ATOM | 1471 | N | THR | H | 191 | 36.024 | 5.481 | 3.603 | 1.00 | 45.41 | N |
| ATOM | 1472 | CA | THR | H | 191 | 35.776 | 5.869 | 2.207 | 1.00 | 44.81 | C |
| ATOM | 1473 | CB | THR | H | 191 | 34.825 | 4.882 | 1.502 | 1.00 | 45.07 | C |
| ATOM | 1474 | OG1 | THR | H | 191 | 35.070 | 3.553 | 1.974 | 1.00 | 45.32 | O |
| ATOM | 1475 | CG2 | THR | H | 191 | 35.021 | 4.941 | −0.024 | 1.00 | 46.01 | C |
| ATOM | 1476 | C | THR | H | 191 | 35.193 | 7.271 | 2.032 | 1.00 | 43.89 | C |
| ATOM | 1477 | O | THR | H | 191 | 35.813 | 8.120 | 1.392 | 1.00 | 44.08 | O |
| ATOM | 1478 | N | GLN | H | 192 | 33.998 | 7.494 | 2.587 | 1.00 | 42.67 | N |
| ATOM | 1479 | CA | GLN | H | 192 | 33.228 | 8.726 | 2.356 | 1.00 | 41.15 | C |
| ATOM | 1480 | CB | GLN | H | 192 | 31.731 | 8.494 | 2.615 | 1.00 | 41.51 | C |
| ATOM | 1481 | CG | GLN | H | 192 | 30.885 | 8.240 | 1.351 | 1.00 | 41.41 | C |
| ATOM | 1482 | CD | GLN | H | 192 | 30.724 | 9.482 | 0.462 | 1.00 | 41.30 | C |
| ATOM | 1483 | OE1 | GLN | H | 192 | 31.045 | 10.610 | 0.862 | 1.00 | 39.57 | O |
| ATOM | 1484 | NE2 | GLN | H | 192 | 30.220 | 9.268 | −0.753 | 1.00 | 41.03 | N |
| ATOM | 1485 | C | GLN | H | 192 | 33.702 | 9.919 | 3.167 | 1.00 | 40.09 | C |
| ATOM | 1486 | O | GLN | H | 192 | 33.879 | 9.831 | 4.376 | 1.00 | 39.71 | O |
| ATOM | 1487 | N | THR | H | 193 | 33.902 | 11.040 | 2.487 | 1.00 | 38.97 | N |
| ATOM | 1488 | CA | THR | H | 193 | 34.219 | 12.293 | 3.156 | 1.00 | 37.90 | C |
| ATOM | 1489 | CB | THR | H | 193 | 34.935 | 13.264 | 2.209 | 1.00 | 38.17 | C |
| ATOM | 1490 | OG1 | THR | H | 193 | 36.212 | 12.726 | 1.857 | 1.00 | 38.67 | O |
| ATOM | 1491 | CG2 | THR | H | 193 | 35.145 | 14.605 | 2.880 | 1.00 | 38.79 | C |
| ATOM | 1492 | C | THR | H | 193 | 32.943 | 12.947 | 3.687 | 1.00 | 36.63 | C |
| ATOM | 1493 | O | THR | H | 193 | 31.917 | 12.975 | 3.006 | 1.00 | 36.40 | O |
| ATOM | 1494 | N | TYR | H | 194 | 33.021 | 13.468 | 4.907 | 1.00 | 35.04 | N |
| ATOM | 1495 | CA | TYR | H | 194 | 31.888 | 14.127 | 5.540 | 1.00 | 33.24 | C |
| ATOM | 1496 | CB | TYR | H | 194 | 31.433 | 13.338 | 6.760 | 1.00 | 32.56 | C |
| ATOM | 1497 | CG | TYR | H | 194 | 30.841 | 12.006 | 6.395 | 1.00 | 30.21 | C |
| ATOM | 1498 | CD1 | TYR | H | 194 | 31.514 | 10.830 | 6.686 | 1.00 | 28.10 | C |
| ATOM | 1499 | CE1 | TYR | H | 194 | 30.984 | 9.604 | 6.351 | 1.00 | 27.08 | C |
| ATOM | 1500 | CZ | TYR | H | 194 | 29.758 | 9.541 | 5.709 | 1.00 | 27.46 | C |
| ATOM | 1501 | OH | TYR | H | 194 | 29.236 | 8.312 | 5.381 | 1.00 | 26.94 | O |
| ATOM | 1502 | CE2 | TYR | H | 194 | 29.067 | 10.699 | 5.398 | 1.00 | 27.00 | C |
| ATOM | 1503 | CD2 | TYR | H | 194 | 29.613 | 11.923 | 5.743 | 1.00 | 28.14 | C |
| ATOM | 1504 | C | TYR | H | 194 | 32.212 | 15.553 | 5.915 | 1.00 | 32.84 | C |
| ATOM | 1505 | O | TYR | H | 194 | 33.106 | 15.814 | 6.726 | 1.00 | 33.08 | O |
| ATOM | 1506 | N | ILE | H | 195 | 31.486 | 16.480 | 5.307 | 1.00 | 32.33 | N |
| ATOM | 1507 | CA | ILE | H | 195 | 31.712 | 17.895 | 5.531 | 1.00 | 32.06 | C |
| ATOM | 1508 | CB | ILE | H | 195 | 32.305 | 18.574 | 4.288 | 1.00 | 31.97 | C |
| ATOM | 1509 | CG1 | ILE | H | 195 | 33.568 | 17.836 | 3.824 | 1.00 | 32.09 | C |
| ATOM | 1510 | CD1 | ILE | H | 195 | 34.008 | 18.163 | 2.408 | 1.00 | 31.42 | C |
| ATOM | 1511 | CG2 | ILE | H | 195 | 32.594 | 20.049 | 4.575 | 1.00 | 31.89 | C |
| ATOM | 1512 | C | ILE | H | 195 | 30.389 | 18.557 | 5.861 | 1.00 | 32.07 | C |
| ATOM | 1513 | O | ILE | H | 195 | 29.380 | 18.295 | 5.198 | 1.00 | 31.94 | O |
| ATOM | 1514 | N | CYS | H | 196 | 30.382 | 19.404 | 6.887 | 1.00 | 31.68 | N |
| ATOM | 1515 | CA | CYS | H | 196 | 29.186 | 20.168 | 7.162 | 1.00 | 31.85 | C |
| ATOM | 1516 | CB | CYS | H | 196 | 28.810 | 20.124 | 8.640 | 1.00 | 31.86 | C |
| ATOM | 1517 | SG | CYS | H | 196 | 29.912 | 20.995 | 9.757 | 1.00 | 32.81 | S |
| ATOM | 1518 | C | CYS | H | 196 | 29.329 | 21.595 | 6.658 | 1.00 | 31.55 | C |
| ATOM | 1519 | O | CYS | H | 196 | 30.370 | 22.215 | 6.814 | 1.00 | 31.96 | O |
| ATOM | 1520 | N | ASN | H | 197 | 28.270 | 22.104 | 6.047 | 1.00 | 31.27 | N |
| ATOM | 1521 | CA | ASN | H | 197 | 28.280 | 23.437 | 5.468 | 1.00 | 30.84 | C |
| ATOM | 1522 | CB | ASN | H | 197 | 27.822 | 23.382 | 4.004 | 1.00 | 30.66 | C |
| ATOM | 1523 | CG | ASN | H | 197 | 28.243 | 22.089 | 3.307 | 1.00 | 30.50 | C |
| ATOM | 1524 | OD1 | ASN | H | 197 | 27.443 | 21.152 | 3.151 | 1.00 | 29.19 | O |
| ATOM | 1525 | ND2 | ASN | H | 197 | 29.512 | 22.026 | 2.908 | 1.00 | 29.04 | N |
| ATOM | 1526 | C | ASN | H | 197 | 27.391 | 24.351 | 6.292 | 1.00 | 30.65 | C |
| ATOM | 1527 | O | ASN | H | 197 | 26.168 | 24.289 | 6.224 | 1.00 | 30.64 | O |
| ATOM | 1528 | N | VAL | H | 198 | 28.021 | 25.193 | 7.088 | 1.00 | 30.76 | N |
| ATOM | 1529 | CA | VAL | H | 198 | 27.290 | 26.053 | 7.996 | 1.00 | 30.79 | C |
| ATOM | 1530 | CB | VAL | H | 198 | 27.902 | 26.011 | 9.402 | 1.00 | 30.31 | C |
| ATOM | 1531 | CG1 | VAL | H | 198 | 27.163 | 26.945 | 10.338 | 1.00 | 29.38 | C |
| ATOM | 1532 | CG2 | VAL | H | 198 | 27.874 | 24.587 | 9.935 | 1.00 | 29.74 | C |
| ATOM | 1533 | C | VAL | H | 198 | 27.277 | 27.467 | 7.449 | 1.00 | 31.29 | C |
| ATOM | 1534 | O | VAL | H | 198 | 28.328 | 28.037 | 7.171 | 1.00 | 31.52 | O |
| ATOM | 1535 | N | ASN | H | 199 | 26.079 | 28.023 | 7.310 | 1.00 | 31.99 | N |
| ATOM | 1536 | CA | ASN | H | 199 | 25.888 | 29.338 | 6.713 | 1.00 | 33.00 | C |
| ATOM | 1537 | CB | ASN | H | 199 | 25.167 | 29.174 | 5.370 | 1.00 | 33.61 | C |
| ATOM | 1538 | CG | ASN | H | 199 | 25.140 | 30.448 | 4.535 | 1.00 | 35.57 | C |
| ATOM | 1539 | OD1 | ASN | H | 199 | 25.392 | 31.559 | 5.023 | 1.00 | 39.00 | O |
| ATOM | 1540 | ND2 | ASN | H | 199 | 24.818 | 30.287 | 3.258 | 1.00 | 36.00 | N |
| ATOM | 1541 | C | ASN | H | 199 | 25.090 | 30.239 | 7.643 | 1.00 | 33.02 | C |
| ATOM | 1542 | O | ASN | H | 199 | 23.931 | 29.964 | 7.928 | 1.00 | 33.30 | O |
| ATOM | 1543 | N | HIS | H | 200 | 25.725 | 31.306 | 8.125 | 1.00 | 33.28 | N |
| ATOM | 1544 | CA | HIS | H | 200 | 25.083 | 32.282 | 8.998 | 1.00 | 33.25 | C |
| ATOM | 1545 | CB | HIS | H | 200 | 25.828 | 32.385 | 10.336 | 1.00 | 32.54 | C |
| ATOM | 1546 | CG | HIS | H | 200 | 25.238 | 33.369 | 11.303 | 1.00 | 30.19 | C |
| ATOM | 1547 | ND1 | HIS | H | 200 | 25.956 | 34.424 | 11.820 | 1.00 | 29.07 | N |
| ATOM | 1548 | CE1 | HIS | H | 200 | 25.192 | 35.117 | 12.646 | 1.00 | 27.43 | C |
| ATOM | 1549 | NE2 | HIS | H | 200 | 24.005 | 34.544 | 12.691 | 1.00 | 26.56 | N |
| ATOM | 1550 | CD2 | HIS | H | 200 | 24.007 | 33.448 | 11.863 | 1.00 | 28.28 | C |
| ATOM | 1551 | C | HIS | H | 200 | 25.083 | 33.611 | 8.275 | 1.00 | 34.44 | C |
| ATOM | 1552 | O | HIS | H | 200 | 26.019 | 34.408 | 8.409 | 1.00 | 34.98 | O |
| ATOM | 1553 | N | LYS | H | 201 | 24.030 | 33.836 | 7.496 | 1.00 | 35.71 | N |
| ATOM | 1554 | CA | LYS | H | 201 | 23.884 | 35.054 | 6.695 | 1.00 | 36.94 | C |
| ATOM | 1555 | CB | LYS | H | 201 | 22.576 | 35.028 | 5.894 | 1.00 | 37.13 | C |
| ATOM | 1556 | CG | LYS | H | 201 | 22.793 | 34.473 | 4.498 | 1.00 | 38.97 | C |
| ATOM | 1557 | CD | LYS | H | 201 | 21.751 | 33.455 | 4.075 | 1.00 | 40.71 | C |
| ATOM | 1558 | CE | LYS | H | 201 | 22.279 | 32.669 | 2.874 | 1.00 | 41.50 | C |
| ATOM | 1559 | NZ | LYS | H | 201 | 21.216 | 31.876 | 2.184 | 1.00 | 42.71 | N |
| ATOM | 1560 | C | LYS | H | 201 | 24.063 | 36.372 | 7.462 | 1.00 | 37.43 | C |
| ATOM | 1561 | O | LYS | H | 201 | 24.829 | 37.231 | 7.005 | 1.00 | 37.55 | O |
| ATOM | 1562 | N | PRO | H | 202 | 23.398 | 36.529 | 8.636 | 1.00 | 37.89 | N |
| ATOM | 1563 | CA | PRO | H | 202 | 23.481 | 37.811 | 9.350 | 1.00 | 38.22 | C |
| ATOM | 1564 | CB | PRO | H | 202 | 22.849 | 37.495 | 10.704 | 1.00 | 37.80 | C |
| ATOM | 1565 | CG | PRO | H | 202 | 21.849 | 36.474 | 10.401 | 1.00 | 37.63 | C |
| ATOM | 1566 | CD | PRO | H | 202 | 22.460 | 35.612 | 9.317 | 1.00 | 37.87 | C |
| ATOM | 1567 | C | PRO | H | 202 | 24.890 | 38.363 | 9.544 | 1.00 | 38.65 | C |
| ATOM | 1568 | O | PRO | H | 202 | 25.046 | 39.572 | 9.683 | 1.00 | 38.86 | O |
| ATOM | 1569 | N | SER | H | 203 | 25.897 | 37.496 | 9.540 | 1.00 | 39.22 | N |
| ATOM | 1570 | CA | SER | H | 203 | 27.282 | 37.933 | 9.723 | 1.00 | 39.95 | C |
| ATOM | 1571 | CB | SER | H | 203 | 27.879 | 37.264 | 10.957 | 1.00 | 39.83 | C |
| ATOM | 1572 | OG | SER | H | 203 | 27.955 | 35.861 | 10.758 | 1.00 | 40.19 | O |
| ATOM | 1573 | C | SER | H | 203 | 28.174 | 37.649 | 8.507 | 1.00 | 40.34 | C |
| ATOM | 1574 | O | SER | H | 203 | 29.374 | 37.930 | 8.540 | 1.00 | 40.28 | O |
| ATOM | 1575 | N | ASN | H | 204 | 27.585 | 37.096 | 7.446 | 1.00 | 41.00 | N |
| ATOM | 1576 | CA | ASN | H | 204 | 28.332 | 36.647 | 6.248 | 1.00 | 42.03 | C |
| ATOM | 1577 | CB | ASN | H | 204 | 28.999 | 37.819 | 5.492 | 1.00 | 42.09 | C |
| ATOM | 1578 | CG | ASN | H | 204 | 28.040 | 38.965 | 5.204 | 1.00 | 43.23 | C |
| ATOM | 1579 | OD1 | ASN | H | 204 | 27.021 | 38.791 | 4.521 | 1.00 | 43.60 | O |
| ATOM | 1580 | ND2 | ASN | H | 204 | 28.368 | 40.153 | 5.724 | 1.00 | 43.35 | N |
| ATOM | 1581 | C | ASN | H | 204 | 29.374 | 35.560 | 6.531 | 1.00 | 42.17 | C |
| ATOM | 1582 | O | ASN | H | 204 | 30.409 | 35.504 | 5.870 | 1.00 | 42.43 | O |
| ATOM | 1583 | N | THR | H | 205 | 29.104 | 34.705 | 7.515 | 1.00 | 42.55 | N |
| ATOM | 1584 | CA | THR | H | 205 | 29.979 | 33.568 | 7.802 | 1.00 | 42.44 | C |
| ATOM | 1585 | CB | THR | H | 205 | 29.948 | 33.161 | 9.294 | 1.00 | 42.41 | C |
| ATOM | 1586 | OG1 | THR | H | 205 | 29.950 | 34.334 | 10.116 | 1.00 | 42.53 | O |
| ATOM | 1587 | CG2 | THR | H | 205 | 31.156 | 32.304 | 9.654 | 1.00 | 42.08 | C |
| ATOM | 1588 | C | THR | H | 205 | 29.491 | 32.412 | 6.957 | 1.00 | 42.66 | C |
| ATOM | 1589 | O | THR | H | 205 | 28.279 | 32.170 | 6.868 | 1.00 | 42.89 | O |
| ATOM | 1590 | N | LYS | H | 206 | 30.433 | 31.740 | 6.300 | 1.00 | 42.73 | N |
| ATOM | 1591 | CA | LYS | H | 206 | 30.188 | 30.482 | 5.601 | 1.00 | 42.89 | C |
| ATOM | 1592 | CB | LYS | H | 206 | 29.999 | 30.694 | 4.096 | 1.00 | 43.05 | C |
| ATOM | 1593 | CG | LYS | H | 206 | 28.722 | 31.470 | 3.723 | 1.00 | 44.48 | C |
| ATOM | 1594 | CD | LYS | H | 206 | 28.523 | 31.646 | 2.201 | 1.00 | 45.77 | C |
| ATOM | 1595 | CE | LYS | H | 206 | 27.968 | 30.366 | 1.554 | 1.00 | 47.12 | C |
| ATOM | 1596 | NZ | LYS | H | 206 | 27.562 | 30.565 | 0.132 | 1.00 | 47.80 | N |
| ATOM | 1597 | C | LYS | H | 206 | 31.389 | 29.593 | 5.883 | 1.00 | 42.79 | C |
| ATOM | 1598 | O | LYS | H | 206 | 32.532 | 29.979 | 5.625 | 1.00 | 42.96 | O |
| ATOM | 1599 | N | VAL | H | 207 | 31.127 | 28.416 | 6.441 | 1.00 | 42.57 | N |
| ATOM | 1600 | CA | VAL | H | 207 | 32.179 | 27.523 | 6.909 | 1.00 | 42.24 | C |
| ATOM | 1601 | CB | VAL | H | 207 | 32.289 | 27.537 | 8.457 | 1.00 | 42.25 | C |
| ATOM | 1602 | CG1 | VAL | H | 207 | 33.228 | 26.447 | 8.964 | 1.00 | 41.72 | C |
| ATOM | 1603 | CG2 | VAL | H | 207 | 32.738 | 28.902 | 8.959 | 1.00 | 42.32 | C |
| ATOM | 1604 | C | VAL | H | 207 | 31.872 | 26.120 | 6.461 | 1.00 | 42.48 | C |
| ATOM | 1605 | O | VAL | H | 207 | 30.721 | 25.685 | 6.491 | 1.00 | 42.31 | O |
| ATOM | 1606 | N | ASP | H | 208 | 32.909 | 25.417 | 6.031 | 1.00 | 42.81 | N |
| ATOM | 1607 | CA | ASP | H | 208 | 32.802 | 23.996 | 5.791 | 1.00 | 43.51 | C |
| ATOM | 1608 | CB | ASP | H | 208 | 33.163 | 23.661 | 4.342 | 1.00 | 43.68 | C |
| ATOM | 1609 | CG | ASP | H | 208 | 32.362 | 24.480 | 3.334 | 1.00 | 44.85 | C |
| ATOM | 1610 | OD1 | ASP | H | 208 | 31.112 | 24.349 | 3.304 | 1.00 | 45.97 | O |
| ATOM | 1611 | OD2 | ASP | H | 208 | 32.985 | 25.255 | 2.570 | 1.00 | 44.42 | O |
| ATOM | 1612 | C | ASP | H | 208 | 33.755 | 23.323 | 6.761 | 1.00 | 43.66 | C |
| ATOM | 1613 | O | ASP | H | 208 | 34.948 | 23.615 | 6.759 | 1.00 | 44.03 | O |
| ATOM | 1614 | N | LYS | H | 209 | 33.232 | 22.455 | 7.618 | 1.00 | 43.72 | N |
| ATOM | 1615 | CA | LYS | H | 209 | 34.087 | 21.731 | 8.539 | 1.00 | 43.87 | C |
| ATOM | 1616 | CB | LYS | H | 209 | 33.624 | 21.905 | 9.985 | 1.00 | 44.02 | C |
| ATOM | 1617 | CG | LYS | H | 209 | 34.736 | 21.731 | 11.022 | 1.00 | 44.34 | C |
| ATOM | 1618 | CD | LYS | H | 209 | 35.668 | 22.931 | 11.032 | 1.00 | 45.07 | C |
| ATOM | 1619 | CE | LYS | H | 209 | 36.881 | 22.711 | 11.920 | 1.00 | 45.77 | C |
| ATOM | 1620 | NZ | LYS | H | 209 | 37.896 | 23.790 | 11.687 | 1.00 | 46.42 | N |
| ATOM | 1621 | C | LYS | H | 209 | 34.129 | 20.266 | 8.168 | 1.00 | 44.00 | C |
| ATOM | 1622 | O | LYS | H | 209 | 33.090 | 19.625 | 8.031 | 1.00 | 43.75 | O |
| ATOM | 1623 | N | ARG | H | 210 | 35.341 | 19.748 | 7.984 | 1.00 | 44.28 | N |
| ATOM | 1624 | CA | ARG | H | 210 | 35.526 | 18.337 | 7.700 | 1.00 | 44.65 | C |
| ATOM | 1625 | CB | ARG | H | 210 | 36.828 | 18.103 | 6.938 | 1.00 | 44.74 | C |
| ATOM | 1626 | CG | ARG | H | 210 | 36.953 | 16.703 | 6.379 | 1.00 | 45.82 | C |
| ATOM | 1627 | CD | ARG | H | 210 | 37.786 | 16.667 | 5.107 | 1.00 | 49.01 | C |
| ATOM | 1628 | NE | ARG | H | 210 | 38.109 | 15.285 | 4.764 | 1.00 | 50.70 | N |
| ATOM | 1629 | CZ | ARG | H | 210 | 39.230 | 14.661 | 5.118 | 1.00 | 51.87 | C |
| ATOM | 1630 | NH1 | ARG | H | 210 | 40.171 | 15.296 | 5.814 | 1.00 | 52.17 | N |
| ATOM | 1631 | NH2 | ARG | H | 210 | 39.417 | 13.395 | 4.766 | 1.00 | 52.90 | N |
| ATOM | 1632 | C | ARG | H | 210 | 35.490 | 17.533 | 8.994 | 1.00 | 44.64 | C |
| ATOM | 1633 | O | ARG | H | 210 | 36.154 | 17.877 | 9.970 | 1.00 | 44.58 | O |
| ATOM | 1634 | N | VAL | H | 211 | 34.699 | 16.469 | 8.994 | 1.00 | 44.91 | N |
| ATOM | 1635 | CA | VAL | H | 211 | 34.500 | 15.662 | 10.190 | 1.00 | 45.38 | C |
| ATOM | 1636 | CB | VAL | H | 211 | 32.993 | 15.570 | 10.576 | 1.00 | 45.27 | C |
| ATOM | 1637 | CG1 | VAL | H | 211 | 32.806 | 14.811 | 11.874 | 1.00 | 45.01 | C |
| ATOM | 1638 | CG2 | VAL | H | 211 | 32.393 | 16.958 | 10.709 | 1.00 | 44.93 | C |
| ATOM | 1639 | C | VAL | H | 211 | 35.107 | 14.280 | 9.983 | 1.00 | 46.02 | C |
| ATOM | 1640 | O | VAL | H | 211 | 34.523 | 13.426 | 9.317 | 1.00 | 45.86 | O |
| ATOM | 1641 | N | GLU | H | 212 | 36.296 | 14.078 | 10.546 | 1.00 | 47.11 | N |
| ATOM | 1642 | CA | GLU | H | 212 | 37.033 | 12.823 | 10.373 | 1.00 | 48.04 | C |
| ATOM | 1643 | CB | GLU | H | 212 | 38.367 | 13.053 | 9.634 | 1.00 | 48.16 | C |
| ATOM | 1644 | CG | GLU | H | 212 | 39.212 | 14.241 | 10.112 | 1.00 | 50.04 | C |
| ATOM | 1645 | CD | GLU | H | 212 | 40.452 | 14.485 | 9.230 | 1.00 | 52.28 | C |
| ATOM | 1646 | OE1 | GLU | H | 212 | 41.195 | 13.516 | 8.922 | 1.00 | 52.73 | O |
| ATOM | 1647 | OE2 | GLU | H | 212 | 40.687 | 15.655 | 8.852 | 1.00 | 52.56 | O |
| ATOM | 1648 | C | GLU | H | 212 | 37.229 | 12.057 | 11.688 | 1.00 | 48.34 | C |
| ATOM | 1649 | O | GLU | H | 212 | 37.341 | 12.673 | 12.753 | 1.00 | 48.33 | O |
| ATOM | 1650 | N | PRO | H | 213 | 37.246 | 10.709 | 11.611 | 1.00 | 48.85 | N |
| ATOM | 1651 | CA | PRO | H | 213 | 37.483 | 9.790 | 12.733 | 1.00 | 49.36 | C |
| ATOM | 1652 | CB | PRO | H | 213 | 37.752 | 8.459 | 12.038 | 1.00 | 49.26 | C |
| ATOM | 1653 | CG | PRO | H | 213 | 36.973 | 8.536 | 10.790 | 1.00 | 49.38 | C |
| ATOM | 1654 | CD | PRO | H | 213 | 36.965 | 9.973 | 10.362 | 1.00 | 48.71 | C |
| ATOM | 1655 | C | PRO | H | 213 | 38.709 | 10.162 | 13.551 | 1.00 | 49.87 | C |
| ATOM | 1656 | O | PRO | H | 213 | 39.664 | 10.713 | 12.995 | 1.00 | 50.00 | O |
| ATOM | 1657 | N | LYS | H | 214 | 38.674 | 9.855 | 14.851 | 1.00 | 50.32 | N |
| ATOM | 1658 | CA | LYS | H | 214 | 39.803 | 10.093 | 15.758 | 1.00 | 50.80 | C |
| ATOM | 1659 | CB | LYS | H | 214 | 41.053 | 9.339 | 15.270 | 1.00 | 50.74 | C |
| ATOM | 1660 | CG | LYS | H | 214 | 42.127 | 9.169 | 16.319 | 1.00 | 51.44 | C |
| ATOM | 1661 | CD | LYS | H | 214 | 43.447 | 8.720 | 15.705 | 1.00 | 51.74 | C |
| ATOM | 1662 | CE | LYS | H | 214 | 44.135 | 7.714 | 16.610 | 1.00 | 51.93 | C |
| ATOM | 1663 | NZ | LYS | H | 214 | 44.139 | 8.157 | 18.041 | 1.00 | 51.95 | N |
| ATOM | 1664 | C | LYS | H | 214 | 40.099 | 11.595 | 15.907 | 1.00 | 50.97 | C |
| ATOM | 1665 | O | LYS | H | 214 | 40.342 | 12.096 | 17.011 | 1.00 | 50.82 | O |
| ATOM | 1666 | N | ASP | L | 1 | −10.590 | 39.004 | 30.730 | 1.00 | 32.06 | N |
| ATOM | 1667 | CA | ASP | L | 1 | −10.215 | 38.271 | 29.487 | 1.00 | 31.94 | C |
| ATOM | 1668 | CB | ASP | L | 1 | −8.690 | 38.267 | 29.318 | 1.00 | 32.29 | C |
| ATOM | 1669 | CG | ASP | L | 1 | −8.097 | 39.688 | 29.317 | 1.00 | 34.13 | C |
| ATOM | 1670 | OD1 | ASP | L | 1 | −8.810 | 40.640 | 29.722 | 1.00 | 35.04 | O |
| ATOM | 1671 | OD2 | ASP | L | 1 | −6.928 | 39.862 | 28.903 | 1.00 | 35.17 | O |
| ATOM | 1672 | C | ASP | L | 1 | −10.768 | 36.858 | 29.574 | 1.00 | 31.21 | C |
| ATOM | 1673 | O | ASP | L | 1 | −10.582 | 36.186 | 30.588 | 1.00 | 31.53 | O |
| ATOM | 1674 | N | VAL | L | 2 | −11.476 | 36.426 | 28.533 | 1.00 | 29.98 | N |
| ATOM | 1675 | CA | VAL | L | 2 | −12.017 | 35.071 | 28.496 | 1.00 | 29.03 | C |
| ATOM | 1676 | CB | VAL | L | 2 | −13.110 | 34.907 | 27.417 | 1.00 | 29.22 | C |
| ATOM | 1677 | CG1 | VAL | L | 2 | −13.655 | 33.471 | 27.397 | 1.00 | 28.95 | C |
| ATOM | 1678 | CG2 | VAL | L | 2 | −14.232 | 35.892 | 27.661 | 1.00 | 29.35 | C |
| ATOM | 1679 | C | VAL | L | 2 | −10.902 | 34.042 | 28.299 | 1.00 | 28.19 | C |
| ATOM | 1680 | O | VAL | L | 2 | −10.313 | 33.927 | 27.217 | 1.00 | 28.07 | O |
| ATOM | 1681 | N | VAL | L | 3 | −10.626 | 33.307 | 29.368 | 1.00 | 27.00 | N |
| ATOM | 1682 | CA | VAL | L | 3 | −9.617 | 32.260 | 29.369 | 1.00 | 26.24 | C |
| ATOM | 1683 | CB | VAL | L | 3 | −9.157 | 31.917 | 30.827 | 1.00 | 26.80 | C |
| ATOM | 1684 | CG1 | VAL | L | 3 | −8.269 | 30.651 | 30.864 | 1.00 | 26.49 | C |
| ATOM | 1685 | CG2 | VAL | L | 3 | −8.440 | 33.137 | 31.476 | 1.00 | 26.54 | C |
| ATOM | 1686 | C | VAL | L | 3 | −10.167 | 31.019 | 28.672 | 1.00 | 25.38 | C |
| ATOM | 1687 | O | VAL | L | 3 | −11.306 | 30.608 | 28.903 | 1.00 | 25.02 | O |
| ATOM | 1688 | N | MET | L | 4 | −9.340 | 30.435 | 27.815 | 1.00 | 24.34 | N |
| ATOM | 1689 | CA | MET | L | 4 | −9.714 | 29.275 | 27.029 | 1.00 | 23.08 | C |
| ATOM | 1690 | CB | MET | L | 4 | −9.547 | 29.597 | 25.550 | 1.00 | 22.99 | C |
| ATOM | 1691 | CG | MET | L | 4 | −10.205 | 30.893 | 25.121 | 1.00 | 22.06 | C |
| ATOM | 1692 | SD | MET | L | 4 | −11.920 | 30.595 | 24.705 | 1.00 | 20.36 | S |
| ATOM | 1693 | CE | MET | L | 4 | −11.699 | 29.797 | 23.122 | 1.00 | 22.04 | C |
| ATOM | 1694 | C | MET | L | 4 | −8.791 | 28.131 | 27.427 | 1.00 | 22.65 | C |
| ATOM | 1695 | O | MET | L | 4 | −7.577 | 28.269 | 27.349 | 1.00 | 22.57 | O |
| ATOM | 1696 | N | THR | L | 5 | −9.372 | 27.012 | 27.852 | 1.00 | 22.20 | N |
| ATOM | 1697 | CA | THR | L | 5 | −8.613 | 25.881 | 28.378 | 1.00 | 22.03 | C |
| ATOM | 1698 | CB | THR | L | 5 | −9.074 | 25.536 | 29.815 | 1.00 | 22.01 | C |
| ATOM | 1699 | OG1 | THR | L | 5 | −9.052 | 26.718 | 30.617 | 1.00 | 21.82 | O |
| ATOM | 1700 | CG2 | THR | L | 5 | −8.164 | 24.508 | 30.450 | 1.00 | 22.80 | C |
| ATOM | 1701 | C | THR | L | 5 | −8.765 | 24.653 | 27.479 | 1.00 | 21.71 | C |
| ATOM | 1702 | O | THR | L | 5 | −9.866 | 24.138 | 27.307 | 1.00 | 21.97 | O |
| ATOM | 1703 | N | GLN | L | 6 | −7.657 | 24.189 | 26.913 | 1.00 | 21.35 | N |
| ATOM | 1704 | CA | GLN | L | 6 | −7.663 | 23.030 | 26.029 | 1.00 | 21.20 | C |
| ATOM | 1705 | CB | GLN | L | 6 | −6.833 | 23.308 | 24.775 | 1.00 | 21.13 | C |
| ATOM | 1706 | CG | GLN | L | 6 | −7.445 | 24.318 | 23.823 | 1.00 | 21.49 | C |
| ATOM | 1707 | CD | GLN | L | 6 | −6.609 | 24.538 | 22.568 | 1.00 | 21.20 | C |
| ATOM | 1708 | OE1 | GLN | L | 6 | −6.193 | 25.658 | 22.269 | 1.00 | 21.09 | O |
| ATOM | 1709 | NE2 | GLN | L | 6 | −6.366 | 23.470 | 21.828 | 1.00 | 20.72 | N |
| ATOM | 1710 | C | GLN | L | 6 | −7.118 | 21.782 | 26.712 | 1.00 | 21.47 | C |
| ATOM | 1711 | O | GLN | L | 6 | −6.086 | 21.834 | 27.378 | 1.00 | 21.25 | O |
| ATOM | 1712 | N | THR | L | 7 | −7.821 | 20.666 | 26.536 | 1.00 | 21.65 | N |
| ATOM | 1713 | CA | THR | L | 7 | −7.332 | 19.349 | 26.934 | 1.00 | 22.32 | C |
| ATOM | 1714 | CB | THR | L | 7 | −7.937 | 18.875 | 28.281 | 1.00 | 22.30 | C |
| ATOM | 1715 | OG1 | THR | L | 7 | −9.356 | 18.794 | 28.171 | 1.00 | 23.28 | O |
| ATOM | 1716 | CG2 | THR | L | 7 | −7.588 | 19.830 | 29.419 | 1.00 | 22.69 | C |
| ATOM | 1717 | C | THR | L | 7 | −7.614 | 18.307 | 25.834 | 1.00 | 22.72 | C |
| ATOM | 1718 | O | THR | L | 7 | −8.638 | 18.385 | 25.128 | 1.00 | 22.87 | O |
| ATOM | 1719 | N | PRO | L | 8 | −6.701 | 17.337 | 25.659 | 1.00 | 22.96 | N |
| ATOM | 1720 | CA | PRO | L | 8 | −5.429 | 17.191 | 26.373 | 1.00 | 23.03 | C |
| ATOM | 1721 | CB | PRO | L | 8 | −5.072 | 15.724 | 26.115 | 1.00 | 22.71 | C |
| ATOM | 1722 | CG | PRO | L | 8 | −5.601 | 15.476 | 24.726 | 1.00 | 22.48 | C |
| ATOM | 1723 | CD | PRO | L | 8 | −6.870 | 16.298 | 24.619 | 1.00 | 22.22 | C |
| ATOM | 1724 | C | PRO | L | 8 | −4.359 | 18.119 | 25.779 | 1.00 | 23.20 | C |
| ATOM | 1725 | O | PRO | L | 8 | −4.546 | 18.644 | 24.694 | 1.00 | 23.42 | O |
| ATOM | 1726 | N | LEU | L | 9 | −3.256 | 18.320 | 26.492 | 1.00 | 23.90 | N |
| ATOM | 1727 | CA | LEU | L | 9 | −2.158 | 19.156 | 26.014 | 1.00 | 24.32 | C |
| ATOM | 1728 | CB | LEU | L | 9 | −1.139 | 19.402 | 27.129 | 1.00 | 24.88 | C |
| ATOM | 1729 | CG | LEU | L | 9 | −1.519 | 20.153 | 28.408 | 1.00 | 26.52 | C |
| ATOM | 1730 | CD1 | LEU | L | 9 | −0.235 | 20.558 | 29.132 | 1.00 | 28.28 | C |
| ATOM | 1731 | CD2 | LEU | L | 9 | −2.357 | 21.373 | 28.119 | 1.00 | 27.02 | C |
| ATOM | 1732 | C | LEU | L | 9 | −1.445 | 18.470 | 24.872 | 1.00 | 24.09 | C |
| ATOM | 1733 | O | LEU | L | 9 | −0.880 | 19.107 | 23.994 | 1.00 | 23.92 | O |
| ATOM | 1734 | N | SER | L | 10 | −1.491 | 17.148 | 24.902 | 1.00 | 24.24 | N |
| ATOM | 1735 | CA | SER | L | 10 | −0.748 | 16.317 | 23.992 | 1.00 | 23.98 | C |
| ATOM | 1736 | CB | SER | L | 10 | 0.535 | 15.891 | 24.697 | 1.00 | 23.87 | C |
| ATOM | 1737 | OG | SER | L | 10 | 0.994 | 14.656 | 24.207 | 1.00 | 26.41 | O |
| ATOM | 1738 | C | SER | L | 10 | −1.613 | 15.112 | 23.626 | 1.00 | 23.60 | C |
| ATOM | 1739 | O | SER | L | 10 | −2.184 | 14.469 | 24.498 | 1.00 | 23.10 | O |
| ATOM | 1740 | N | LEU | L | 11 | −1.729 | 14.818 | 22.334 | 1.00 | 23.71 | N |
| ATOM | 1741 | CA | LEU | L | 11 | −2.568 | 13.701 | 21.888 | 1.00 | 23.72 | C |
| ATOM | 1742 | CB | LEU | L | 11 | −3.943 | 14.186 | 21.419 | 1.00 | 23.49 | C |
| ATOM | 1743 | CG | LEU | L | 11 | −4.939 | 13.131 | 20.922 | 1.00 | 24.96 | C |
| ATOM | 1744 | CD1 | LEU | L | 11 | −5.334 | 12.103 | 22.008 | 1.00 | 25.28 | C |
| ATOM | 1745 | CD2 | LEU | L | 11 | −6.167 | 13.806 | 20.346 | 1.00 | 25.24 | C |
| ATOM | 1746 | C | LEU | L | 11 | −1.902 | 12.840 | 20.815 | 1.00 | 23.84 | C |
| ATOM | 1747 | O | LEU | L | 11 | −1.947 | 13.176 | 19.624 | 1.00 | 23.98 | O |
| ATOM | 1748 | N | PRO | L | 12 | −1.269 | 11.729 | 21.239 | 1.00 | 23.69 | N |
| ATOM | 1749 | CA | PRO | L | 12 | −0.757 | 10.764 | 20.285 | 1.00 | 23.39 | C |
| ATOM | 1750 | CB | PRO | L | 12 | 0.101 | 9.826 | 21.144 | 1.00 | 23.36 | C |
| ATOM | 1751 | CG | PRO | L | 12 | 0.376 | 10.585 | 22.408 | 1.00 | 23.92 | C |
| ATOM | 1752 | CD | PRO | L | 12 | −0.888 | 11.374 | 22.618 | 1.00 | 24.03 | C |
| ATOM | 1753 | C | PRO | L | 12 | −1.915 | 10.014 | 19.674 | 1.00 | 23.18 | C |
| ATOM | 1754 | O | PRO | L | 12 | −2.794 | 9.538 | 20.387 | 1.00 | 23.54 | O |
| ATOM | 1755 | N | VAL | L | 13 | −1.922 | 9.936 | 18.355 | 1.00 | 22.83 | N |
| ATOM | 1756 | CA | VAL | L | 13 | −2.988 | 9.281 | 17.633 | 1.00 | 22.70 | C |
| ATOM | 1757 | CB | VAL | L | 13 | −3.845 | 10.289 | 16.858 | 1.00 | 22.84 | C |
| ATOM | 1758 | CG1 | VAL | L | 13 | −5.050 | 9.589 | 16.314 | 1.00 | 24.00 | C |
| ATOM | 1759 | CG2 | VAL | L | 13 | −4.296 | 11.426 | 17.752 | 1.00 | 23.45 | C |
| ATOM | 1760 | C | VAL | L | 13 | −2.406 | 8.293 | 16.631 | 1.00 | 22.45 | C |
| ATOM | 1761 | O | VAL | L | 13 | −1.325 | 8.512 | 16.081 | 1.00 | 22.54 | O |
| ATOM | 1762 | N | THR | L | 14 | −3.134 | 7.208 | 16.394 | 1.00 | 22.15 | N |
| ATOM | 1763 | CA | THR | L | 14 | −2.729 | 6.212 | 15.427 | 1.00 | 22.20 | C |
| ATOM | 1764 | CB | THR | L | 14 | −2.836 | 4.804 | 16.029 | 1.00 | 22.55 | C |
| ATOM | 1765 | OG1 | THR | L | 14 | −2.033 | 4.755 | 17.224 | 1.00 | 23.81 | O |
| ATOM | 1766 | CG2 | THR | L | 14 | −2.351 | 3.727 | 15.049 | 1.00 | 22.41 | C |
| ATOM | 1767 | C | THR | L | 14 | −3.559 | 6.389 | 14.153 | 1.00 | 21.86 | C |
| ATOM | 1768 | O | THR | L | 14 | −4.788 | 6.358 | 14.194 | 1.00 | 21.92 | O |
| ATOM | 1769 | N | PRO | L | 15 | −2.885 | 6.621 | 13.017 | 1.00 | 21.63 | N |
| ATOM | 1770 | CA | PRO | L | 15 | −3.627 | 6.797 | 11.788 | 1.00 | 21.69 | C |
| ATOM | 1771 | CB | PRO | L | 15 | −2.563 | 6.578 | 10.713 | 1.00 | 21.55 | C |
| ATOM | 1772 | CG | PRO | L | 15 | −1.320 | 7.079 | 11.347 | 1.00 | 21.26 | C |
| ATOM | 1773 | CD | PRO | L | 15 | −1.429 | 6.733 | 12.803 | 1.00 | 21.61 | C |
| ATOM | 1774 | C | PRO | L | 15 | −4.737 | 5.759 | 11.690 | 1.00 | 21.74 | C |
| ATOM | 1775 | O | PRO | L | 15 | −4.496 | 4.558 | 11.882 | 1.00 | 21.66 | O |
| ATOM | 1776 | N | GLY | L | 16 | −5.945 | 6.239 | 11.423 | 1.00 | 21.74 | N |
| ATOM | 1777 | CA | GLY | L | 16 | −7.118 | 5.384 | 11.367 | 1.00 | 21.89 | C |
| ATOM | 1778 | C | GLY | L | 16 | −8.058 | 5.584 | 12.540 | 1.00 | 22.02 | C |
| ATOM | 1779 | O | GLY | L | 16 | −9.261 | 5.410 | 12.396 | 1.00 | 21.45 | O |
| ATOM | 1780 | N | GLU | L | 17 | −7.520 | 5.959 | 13.701 | 1.00 | 22.49 | N |
| ATOM | 1781 | CA | GLU | L | 17 | −8.361 | 6.132 | 14.887 | 1.00 | 23.36 | C |
| ATOM | 1782 | CB | GLU | L | 17 | −7.597 | 5.734 | 16.153 | 1.00 | 23.22 | C |
| ATOM | 1783 | CG | GLU | L | 17 | −6.682 | 6.775 | 16.724 | 1.00 | 24.92 | C |
| ATOM | 1784 | CD | GLU | L | 17 | −6.075 | 6.346 | 18.047 | 1.00 | 26.28 | C |
| ATOM | 1785 | OE1 | GLU | L | 17 | −4.893 | 6.658 | 18.313 | 1.00 | 27.27 | O |
| ATOM | 1786 | OE2 | GLU | L | 17 | −6.784 | 5.685 | 18.828 | 1.00 | 28.34 | O |
| ATOM | 1787 | C | GLU | L | 17 | −9.020 | 7.527 | 14.998 | 1.00 | 23.62 | C |
| ATOM | 1788 | O | GLU | L | 17 | −8.518 | 8.501 | 14.421 | 1.00 | 24.00 | O |
| ATOM | 1789 | N | PRO | L | 18 | −10.163 | 7.625 | 15.709 | 1.00 | 23.58 | N |
| ATOM | 1790 | CA | PRO | L | 18 | −10.794 | 8.942 | 15.878 | 1.00 | 23.48 | C |
| ATOM | 1791 | CB | PRO | L | 18 | −12.250 | 8.608 | 16.224 | 1.00 | 23.48 | C |
| ATOM | 1792 | CG | PRO | L | 18 | −12.232 | 7.223 | 16.766 | 1.00 | 23.81 | C |
| ATOM | 1793 | CD | PRO | L | 18 | −10.932 | 6.549 | 16.363 | 1.00 | 23.53 | C |
| ATOM | 1794 | C | PRO | L | 18 | −10.145 | 9.751 | 16.999 | 1.00 | 23.41 | C |
| ATOM | 1795 | O | PRO | L | 18 | −9.719 | 9.182 | 18.000 | 1.00 | 23.82 | O |
| ATOM | 1796 | N | ALA | L | 19 | −10.047 | 11.063 | 16.824 | 1.00 | 22.86 | N |
| ATOM | 1797 | CA | ALA | L | 19 | −9.498 | 11.912 | 17.865 | 1.00 | 22.43 | C |
| ATOM | 1798 | CB | ALA | L | 19 | −8.218 | 12.563 | 17.396 | 1.00 | 22.45 | C |
| ATOM | 1799 | C | ALA | L | 19 | −10.506 | 12.958 | 18.341 | 1.00 | 22.30 | C |
| ATOM | 1800 | O | ALA | L | 19 | −11.474 | 13.278 | 17.637 | 1.00 | 22.23 | O |
| ATOM | 1801 | N | SER | L | 20 | −10.242 | 13.508 | 19.522 | 1.00 | 21.93 | N |
| ATOM | 1802 | CA | SER | L | 20 | −11.172 | 14.379 | 20.213 | 1.00 | 22.11 | C |
| ATOM | 1803 | CB | SER | L | 20 | −12.085 | 13.530 | 21.090 | 1.00 | 22.51 | C |
| ATOM | 1804 | OG | SER | L | 20 | −13.385 | 14.067 | 21.135 | 1.00 | 25.46 | O |
| ATOM | 1805 | C | SER | L | 20 | −10.418 | 15.409 | 21.064 | 1.00 | 21.68 | C |
| ATOM | 1806 | O | SER | L | 20 | −9.657 | 15.059 | 21.972 | 1.00 | 21.63 | O |
| ATOM | 1807 | N | ILE | L | 21 | −10.624 | 16.680 | 20.741 | 1.00 | 21.31 | N |
| ATOM | 1808 | CA | ILE | L | 21 | −9.960 | 17.789 | 21.413 | 1.00 | 20.49 | C |
| ATOM | 1809 | CB | ILE | L | 21 | −9.134 | 18.655 | 20.432 | 1.00 | 20.86 | C |
| ATOM | 1810 | CG1 | ILE | L | 21 | −8.167 | 17.781 | 19.652 | 1.00 | 22.15 | C |
| ATOM | 1811 | CD1 | ILE | L | 21 | −7.296 | 18.566 | 18.715 | 1.00 | 23.83 | C |
| ATOM | 1812 | CG2 | ILE | L | 21 | −8.348 | 19.778 | 21.182 | 1.00 | 21.20 | C |
| ATOM | 1813 | C | ILE | L | 21 | −11.030 | 18.652 | 22.037 | 1.00 | 19.65 | C |
| ATOM | 1814 | O | ILE | L | 21 | −12.026 | 18.978 | 21.397 | 1.00 | 18.91 | O |
| ATOM | 1815 | N | SER | L | 22 | −10.801 | 19.025 | 23.290 | 1.00 | 19.02 | N |
| ATOM | 1816 | CA | SER | L | 22 | −11.781 | 19.751 | 24.074 | 1.00 | 18.57 | C |
| ATOM | 1817 | CB | SER | L | 22 | −12.087 | 18.956 | 25.352 | 1.00 | 18.26 | C |
| ATOM | 1818 | OG | SER | L | 22 | −12.591 | 19.785 | 26.384 | 1.00 | 18.63 | O |
| ATOM | 1819 | C | SER | L | 22 | −11.313 | 21.186 | 24.372 | 1.00 | 18.22 | C |
| ATOM | 1820 | O | SER | L | 22 | −10.134 | 21.411 | 24.653 | 1.00 | 18.25 | O |
| ATOM | 1821 | N | CYS | L | 23 | −12.232 | 22.145 | 24.282 | 1.00 | 17.93 | N |
| ATOM | 1822 | CA | CYS | L | 23 | −11.942 | 23.538 | 24.628 | 1.00 | 18.73 | C |
| ATOM | 1823 | CB | CYS | L | 23 | −11.860 | 24.438 | 23.371 | 1.00 | 18.51 | C |
| ATOM | 1824 | SG | CYS | L | 23 | −11.496 | 26.246 | 23.659 | 1.00 | 19.59 | S |
| ATOM | 1825 | C | CYS | L | 23 | −13.030 | 24.038 | 25.567 | 1.00 | 19.02 | C |
| ATOM | 1826 | O | CYS | L | 23 | −14.218 | 23.973 | 25.239 | 1.00 | 19.32 | O |
| ATOM | 1827 | N | THR | L | 24 | −12.617 | 24.519 | 26.736 | 1.00 | 19.21 | N |
| ATOM | 1828 | CA | THR | L | 24 | −13.526 | 25.111 | 27.706 | 1.00 | 19.50 | C |
| ATOM | 1829 | CB | THR | L | 24 | −13.418 | 24.418 | 29.101 | 1.00 | 19.63 | C |
| ATOM | 1830 | OG1 | THR | L | 24 | −13.876 | 23.068 | 29.007 | 1.00 | 20.55 | O |
| ATOM | 1831 | CG2 | THR | L | 24 | −14.289 | 25.125 | 30.143 | 1.00 | 20.48 | C |
| ATOM | 1832 | C | THR | L | 24 | −13.212 | 26.598 | 27.856 | 1.00 | 19.64 | C |
| ATOM | 1833 | O | THR | L | 24 | −12.049 | 26.984 | 28.079 | 1.00 | 19.27 | O |
| ATOM | 1834 | N | SER | L | 25 | −14.246 | 27.428 | 27.745 | 1.00 | 19.44 | N |
| ATOM | 1835 | CA | SER | L | 25 | −14.096 | 28.856 | 28.031 | 1.00 | 19.83 | C |
| ATOM | 1836 | CB | SER | L | 25 | −14.796 | 29.713 | 26.973 | 1.00 | 19.25 | C |
| ATOM | 1837 | OG | SER | L | 25 | −16.188 | 29.495 | 26.980 | 1.00 | 18.63 | O |
| ATOM | 1838 | C | SER | L | 25 | −14.571 | 29.227 | 29.437 | 1.00 | 20.33 | C |
| ATOM | 1839 | O | SER | L | 25 | −15.589 | 28.724 | 29.909 | 1.00 | 20.43 | O |
| ATOM | 1840 | N | GLY | L | 26 | −13.820 | 30.114 | 30.093 | 1.00 | 21.08 | N |
| ATOM | 1841 | CA | GLY | L | 26 | −14.161 | 30.619 | 31.428 | 1.00 | 21.59 | C |
| ATOM | 1842 | C | GLY | L | 26 | −15.458 | 31.419 | 31.504 | 1.00 | 22.20 | C |
| ATOM | 1843 | O | GLY | L | 26 | −15.945 | 31.720 | 32.597 | 1.00 | 22.19 | O |
| ATOM | 1844 | N | GLN | L | 27 | −16.030 | 31.751 | 30.348 | 1.00 | 22.41 | N |
| ATOM | 1845 | CA | GLN | L | 27 | −17.281 | 32.489 | 30.295 | 1.00 | 22.93 | C |
| ATOM | 1846 | CB | GLN | L | 27 | −16.985 | 33.994 | 30.461 | 1.00 | 23.50 | C |
| ATOM | 1847 | CG | GLN | L | 27 | −18.066 | 34.990 | 30.020 | 1.00 | 26.48 | C |
| ATOM | 1848 | CD | GLN | L | 27 | −17.567 | 36.445 | 30.075 | 1.00 | 30.85 | C |
| ATOM | 1849 | OE1 | GLN | L | 27 | −16.389 | 36.701 | 30.350 | 1.00 | 33.22 | O |
| ATOM | 1850 | NE2 | GLN | L | 27 | −18.464 | 37.396 | 29.814 | 1.00 | 30.73 | N |
| ATOM | 1851 | C | GLN | L | 27 | −17.968 | 32.158 | 28.972 | 1.00 | 22.61 | C |
| ATOM | 1852 | O | GLN | L | 27 | −17.313 | 31.804 | 27.997 | 1.00 | 22.90 | O |
| ATOM | 1853 | N | SER | L | 27A | −19.292 | 32.259 | 28.941 | 1.00 | 22.29 | N |
| ATOM | 1854 | CA | SER | L | 27A | −20.056 | 31.919 | 27.748 | 1.00 | 21.71 | C |
| ATOM | 1855 | CB | SER | L | 27A | −21.532 | 32.168 | 27.984 | 1.00 | 21.16 | C |
| ATOM | 1856 | OG | SER | L | 27A | −22.234 | 31.928 | 26.780 | 1.00 | 21.71 | O |
| ATOM | 1857 | C | SER | L | 27A | −19.604 | 32.651 | 26.471 | 1.00 | 21.56 | C |
| ATOM | 1858 | O | SER | L | 27A | −19.381 | 33.862 | 26.469 | 1.00 | 21.41 | O |
| ATOM | 1859 | N | LEU | L | 27B | −19.491 | 31.905 | 25.382 | 1.00 | 21.38 | N |
| ATOM | 1860 | CA | LEU | L | 27B | −19.085 | 32.490 | 24.107 | 1.00 | 21.23 | C |
| ATOM | 1861 | CB | LEU | L | 27B | −18.130 | 31.566 | 23.354 | 1.00 | 20.60 | C |
| ATOM | 1862 | CG | LEU | L | 27B | −16.846 | 31.185 | 24.089 | 1.00 | 21.05 | C |
| ATOM | 1863 | CD1 | LEU | L | 27B | −16.100 | 30.136 | 23.292 | 1.00 | 20.88 | C |
| ATOM | 1864 | CD2 | LEU | L | 27B | −15.937 | 32.379 | 24.393 | 1.00 | 18.78 | C |
| ATOM | 1865 | C | LEU | L | 27B | −20.260 | 32.890 | 23.218 | 1.00 | 21.34 | C |
| ATOM | 1866 | O | LEU | L | 27B | −20.052 | 33.415 | 22.129 | 1.00 | 21.41 | O |
| ATOM | 1867 | N | VAL | L | 27C | −21.484 | 32.624 | 23.677 | 1.00 | 21.44 | N |
| ATOM | 1868 | CA | VAL | L | 27C | −22.688 | 33.094 | 22.993 | 1.00 | 21.36 | C |
| ATOM | 1869 | CB | VAL | L | 27C | −23.991 | 32.559 | 23.663 | 1.00 | 21.73 | C |
| ATOM | 1870 | CG1 | VAL | L | 27C | −25.227 | 33.116 | 22.940 | 1.00 | 21.50 | C |
| ATOM | 1871 | CG2 | VAL | L | 27C | −24.030 | 31.017 | 23.688 | 1.00 | 20.76 | C |
| ATOM | 1872 | C | VAL | L | 27C | −22.723 | 34.624 | 22.985 | 1.00 | 21.20 | C |
| ATOM | 1873 | O | VAL | L | 27C | −22.747 | 35.261 | 24.038 | 1.00 | 20.87 | O |
| ATOM | 1874 | N | HIS | L | 27D | −22.712 | 35.200 | 21.790 | 1.00 | 21.39 | N |
| ATOM | 1875 | CA | HIS | L | 27D | −22.748 | 36.662 | 21.608 | 1.00 | 21.92 | C |
| ATOM | 1876 | CB | HIS | L | 27D | −22.393 | 36.993 | 20.144 | 1.00 | 21.72 | C |
| ATOM | 1877 | CG | HIS | L | 27D | −21.953 | 38.407 | 19.914 | 1.00 | 21.85 | C |
| ATOM | 1878 | ND1 | HIS | L | 27D | −22.833 | 39.419 | 19.599 | 1.00 | 22.27 | N |
| ATOM | 1879 | CE1 | HIS | L | 27D | −22.166 | 40.552 | 19.451 | 1.00 | 20.54 | C |
| ATOM | 1880 | NE2 | HIS | L | 27D | −20.883 | 40.305 | 19.633 | 1.00 | 20.83 | N |
| ATOM | 1881 | CD2 | HIS | L | 27D | −20.721 | 38.971 | 19.923 | 1.00 | 21.63 | C |
| ATOM | 1882 | C | HIS | L | 27D | −24.135 | 37.231 | 21.967 | 1.00 | 22.19 | C |
| ATOM | 1883 | O | HIS | L | 27D | −25.128 | 36.490 | 22.013 | 1.00 | 21.57 | O |
| ATOM | 1884 | N | ILE | L | 27E | −24.212 | 38.544 | 22.200 | 1.00 | 22.79 | N |
| ATOM | 1885 | CA | ILE | L | 27E | −25.520 | 39.189 | 22.365 | 1.00 | 23.39 | C |
| ATOM | 1886 | CB | ILE | L | 27E | −25.412 | 40.641 | 22.902 | 1.00 | 23.81 | C |
| ATOM | 1887 | CG1 | ILE | L | 27E | −26.774 | 41.168 | 23.427 | 1.00 | 26.40 | C |
| ATOM | 1888 | CD1 | ILE | L | 27E | −27.684 | 40.169 | 24.265 | 1.00 | 28.82 | C |
| ATOM | 1889 | CG2 | ILE | L | 27E | −24.825 | 41.599 | 21.831 | 1.00 | 24.24 | C |
| ATOM | 1890 | C | ILE | L | 27E | −26.391 | 39.065 | 21.087 | 1.00 | 23.11 | C |
| ATOM | 1891 | O | ILE | L | 27E | −27.618 | 39.086 | 21.163 | 1.00 | 23.55 | O |
| ATOM | 1892 | N | ASN | L | 28 | −25.754 | 38.869 | 19.936 | 1.00 | 22.33 | N |
| ATOM | 1893 | CA | ASN | L | 28 | −26.479 | 38.682 | 18.686 | 1.00 | 21.79 | C |
| ATOM | 1894 | CB | ASN | L | 28 | −25.651 | 39.207 | 17.499 | 1.00 | 22.26 | C |
| ATOM | 1895 | CG | ASN | L | 28 | −24.591 | 38.216 | 17.008 | 1.00 | 22.23 | C |
| ATOM | 1896 | OD1 | ASN | L | 28 | −24.449 | 37.103 | 17.534 | 1.00 | 22.15 | O |
| ATOM | 1897 | ND2 | ASN | L | 28 | −23.841 | 38.627 | 15.979 | 1.00 | 20.20 | N |
| ATOM | 1898 | C | ASN | L | 28 | −26.953 | 37.252 | 18.432 | 1.00 | 21.33 | C |
| ATOM | 1899 | O | ASN | L | 28 | −27.590 | 36.981 | 17.426 | 1.00 | 20.24 | O |
| ATOM | 1900 | N | GLY | L | 29 | −26.615 | 36.339 | 19.342 | 1.00 | 21.46 | N |
| ATOM | 1901 | CA | GLY | L | 29 | −27.121 | 34.972 | 19.277 | 1.00 | 20.88 | C |
| ATOM | 1902 | C | GLY | L | 29 | −26.124 | 33.968 | 18.745 | 1.00 | 20.91 | C |
| ATOM | 1903 | O | GLY | L | 29 | −26.246 | 32.776 | 19.029 | 1.00 | 21.49 | O |
| ATOM | 1904 | N | ASN | L | 30 | −25.151 | 34.435 | 17.960 | 1.00 | 19.92 | N |
| ATOM | 1905 | CA | ASN | L | 30 | −24.132 | 33.561 | 17.397 | 1.00 | 19.01 | C |
| ATOM | 1906 | CB | ASN | L | 30 | −23.505 | 34.208 | 16.162 | 1.00 | 19.58 | C |
| ATOM | 1907 | CG | ASN | L | 30 | −24.355 | 34.036 | 14.905 | 1.00 | 20.39 | C |
| ATOM | 1908 | OD1 | ASN | L | 30 | −25.415 | 33.429 | 14.946 | 1.00 | 22.73 | O |
| ATOM | 1909 | ND2 | ASN | L | 30 | −23.878 | 34.566 | 13.784 | 1.00 | 19.89 | N |
| ATOM | 1910 | C | ASN | L | 30 | −23.032 | 33.206 | 18.398 | 1.00 | 18.37 | C |
| ATOM | 1911 | O | ASN | L | 30 | −22.772 | 33.968 | 19.338 | 1.00 | 18.33 | O |
| ATOM | 1912 | N | THR | L | 31 | −22.381 | 32.061 | 18.185 | 1.00 | 17.43 | N |
| ATOM | 1913 | CA | THR | L | 31 | −21.263 | 31.623 | 19.019 | 1.00 | 16.87 | C |
| ATOM | 1914 | CB | THR | L | 31 | −21.525 | 30.242 | 19.636 | 1.00 | 17.27 | C |
| ATOM | 1915 | OG1 | THR | L | 31 | −22.831 | 30.232 | 20.236 | 1.00 | 18.84 | O |
| ATOM | 1916 | CG2 | THR | L | 31 | −20.464 | 29.923 | 20.711 | 1.00 | 16.26 | C |
| ATOM | 1917 | C | THR | L | 31 | −19.973 | 31.588 | 18.203 | 1.00 | 16.35 | C |
| ATOM | 1918 | O | THR | L | 31 | −19.817 | 30.746 | 17.321 | 1.00 | 15.81 | O |
| ATOM | 1919 | N | TYR | L | 32 | −19.049 | 32.499 | 18.507 | 1.00 | 15.49 | N |
| ATOM | 1920 | CA | TYR | L | 32 | −17.868 | 32.667 | 17.670 | 1.00 | 14.73 | C |
| ATOM | 1921 | CB | TYR | L | 32 | −17.557 | 34.150 | 17.409 | 1.00 | 14.88 | C |
| ATOM | 1922 | CG | TYR | L | 32 | −18.686 | 34.945 | 16.767 | 1.00 | 13.72 | C |
| ATOM | 1923 | CD1 | TYR | L | 32 | −18.833 | 35.000 | 15.383 | 1.00 | 13.21 | C |
| ATOM | 1924 | CE1 | TYR | L | 32 | −19.855 | 35.716 | 14.794 | 1.00 | 14.08 | C |
| ATOM | 1925 | CZ | TYR | L | 32 | −20.744 | 36.423 | 15.598 | 1.00 | 15.98 | C |
| ATOM | 1926 | OH | TYR | L | 32 | −21.773 | 37.151 | 15.035 | 1.00 | 17.80 | O |
| ATOM | 1927 | CE2 | TYR | L | 32 | −20.609 | 36.407 | 16.968 | 1.00 | 14.48 | C |
| ATOM | 1928 | CD2 | TYR | L | 32 | −19.581 | 35.664 | 17.548 | 1.00 | 14.26 | C |
| ATOM | 1929 | C | TYR | L | 32 | −16.686 | 31.922 | 18.280 | 1.00 | 14.65 | C |
| ATOM | 1930 | O | TYR | L | 32 | −15.708 | 32.516 | 18.759 | 1.00 | 14.34 | O |
| ATOM | 1931 | N | LEU | L | 33 | −16.811 | 30.596 | 18.275 | 1.00 | 14.38 | N |
| ATOM | 1932 | CA | LEU | L | 33 | −15.714 | 29.703 | 18.623 | 1.00 | 13.63 | C |
| ATOM | 1933 | CB | LEU | L | 33 | −16.192 | 28.680 | 19.658 | 1.00 | 13.58 | C |
| ATOM | 1934 | CG | LEU | L | 33 | −15.222 | 27.740 | 20.390 | 1.00 | 12.99 | C |
| ATOM | 1935 | CD1 | LEU | L | 33 | −13.933 | 28.431 | 20.796 | 1.00 | 10.36 | C |
| ATOM | 1936 | CD2 | LEU | L | 33 | −14.953 | 26.481 | 19.571 | 1.00 | 13.46 | C |
| ATOM | 1937 | C | LEU | L | 33 | −15.177 | 29.036 | 17.351 | 1.00 | 13.17 | C |
| ATOM | 1938 | O | LEU | L | 33 | −15.928 | 28.430 | 16.585 | 1.00 | 12.70 | O |
| ATOM | 1939 | N | HIS | L | 34 | −13.874 | 29.177 | 17.129 | 1.00 | 12.97 | N |
| ATOM | 1940 | CA | HIS | L | 34 | −13.219 | 28.662 | 15.918 | 1.00 | 12.74 | C |
| ATOM | 1941 | CB | HIS | L | 34 | −12.710 | 29.819 | 15.032 | 1.00 | 12.44 | C |
| ATOM | 1942 | CG | HIS | L | 34 | −13.536 | 31.067 | 15.126 | 1.00 | 12.31 | C |
| ATOM | 1943 | ND1 | HIS | L | 34 | −14.859 | 31.117 | 14.741 | 1.00 | 10.78 | N |
| ATOM | 1944 | CE1 | HIS | L | 34 | −15.327 | 32.335 | 14.951 | 1.00 | 11.56 | C |
| ATOM | 1945 | NE2 | HIS | L | 34 | −14.355 | 33.080 | 15.448 | 1.00 | 9.97 | N |
| ATOM | 1946 | CD2 | HIS | L | 34 | −13.227 | 32.308 | 15.577 | 1.00 | 11.36 | C |
| ATOM | 1947 | C | HIS | L | 34 | −12.062 | 27.723 | 16.259 | 1.00 | 12.44 | C |
| ATOM | 1948 | O | HIS | L | 34 | −11.460 | 27.854 | 17.335 | 1.00 | 12.75 | O |
| ATOM | 1949 | N | TRP | L | 35 | −11.750 | 26.797 | 15.345 | 1.00 | 11.73 | N |
| ATOM | 1950 | CA | TRP | L | 35 | −10.610 | 25.876 | 15.516 | 1.00 | 11.46 | C |
| ATOM | 1951 | CB | TRP | L | 35 | −11.074 | 24.424 | 15.529 | 1.00 | 10.71 | C |
| ATOM | 1952 | CG | TRP | L | 35 | −11.902 | 24.039 | 16.678 | 1.00 | 9.60 | C |
| ATOM | 1953 | CD1 | TRP | L | 35 | −13.267 | 24.042 | 16.741 | 1.00 | 8.74 | C |
| ATOM | 1954 | NE1 | TRP | L | 35 | −13.689 | 23.585 | 17.961 | 1.00 | 8.69 | N |
| ATOM | 1955 | CE2 | TRP | L | 35 | −12.590 | 23.288 | 18.728 | 1.00 | 9.08 | C |
| ATOM | 1956 | CD2 | TRP | L | 35 | −11.445 | 23.546 | 17.944 | 1.00 | 9.32 | C |
| ATOM | 1957 | CE3 | TRP | L | 35 | −10.184 | 23.297 | 18.490 | 1.00 | 7.72 | C |
| ATOM | 1958 | CZ3 | TRP | L | 35 | −10.101 | 22.817 | 19.777 | 1.00 | 7.90 | C |
| ATOM | 1959 | CH2 | TRP | L | 35 | −11.253 | 22.561 | 20.533 | 1.00 | 9.05 | C |
| ATOM | 1960 | CZ2 | TRP | L | 35 | −12.508 | 22.795 | 20.029 | 1.00 | 8.86 | C |
| ATOM | 1961 | C | TRP | L | 35 | −9.566 | 26.042 | 14.407 | 1.00 | 11.93 | C |
| ATOM | 1962 | O | TRP | L | 35 | −9.916 | 26.102 | 13.219 | 1.00 | 11.99 | O |
| ATOM | 1963 | N | TYR | L | 36 | −8.292 | 26.097 | 14.804 | 1.00 | 12.65 | N |
| ATOM | 1964 | CA | TYR | L | 36 | −7.157 | 26.218 | 13.872 | 1.00 | 13.17 | C |
| ATOM | 1965 | CB | TYR | L | 36 | −6.383 | 27.519 | 14.102 | 1.00 | 12.90 | C |
| ATOM | 1966 | CG | TYR | L | 36 | −7.159 | 28.765 | 13.771 | 1.00 | 14.16 | C |
| ATOM | 1967 | CD1 | TYR | L | 36 | −6.952 | 29.448 | 12.561 | 1.00 | 14.77 | C |
| ATOM | 1968 | CE1 | TYR | L | 36 | −7.682 | 30.597 | 12.252 | 1.00 | 13.81 | C |
| ATOM | 1969 | CZ | TYR | L | 36 | −8.616 | 31.071 | 13.155 | 1.00 | 13.98 | C |
| ATOM | 1970 | OH | TYR | L | 36 | −9.350 | 32.197 | 12.876 | 1.00 | 15.85 | O |
| ATOM | 1971 | CE2 | TYR | L | 36 | −8.828 | 30.426 | 14.356 | 1.00 | 14.02 | C |
| ATOM | 1972 | CD2 | TYR | L | 36 | −8.111 | 29.273 | 14.657 | 1.00 | 14.92 | C |
| ATOM | 1973 | C | TYR | L | 36 | −6.186 | 25.068 | 14.038 | 1.00 | 13.59 | C |
| ATOM | 1974 | O | TYR | L | 36 | −6.075 | 24.465 | 15.113 | 1.00 | 13.85 | O |
| ATOM | 1975 | N | LEU | L | 37 | −5.477 | 24.769 | 12.964 | 1.00 | 14.42 | N |
| ATOM | 1976 | CA | LEU | L | 37 | −4.353 | 23.856 | 13.027 | 1.00 | 15.27 | C |
| ATOM | 1977 | CB | LEU | L | 37 | −4.604 | 22.617 | 12.180 | 1.00 | 14.75 | C |
| ATOM | 1978 | CG | LEU | L | 37 | −3.405 | 21.730 | 11.856 | 1.00 | 15.02 | C |
| ATOM | 1979 | CD1 | LEU | L | 37 | −2.789 | 21.170 | 13.135 | 1.00 | 16.32 | C |
| ATOM | 1980 | CD2 | LEU | L | 37 | −3.809 | 20.608 | 10.893 | 1.00 | 12.97 | C |
| ATOM | 1981 | C | LEU | L | 37 | −3.111 | 24.585 | 12.554 | 1.00 | 16.06 | C |
| ATOM | 1982 | O | LEU | L | 37 | −3.115 | 25.191 | 11.489 | 1.00 | 16.55 | O |
| ATOM | 1983 | N | GLN | L | 38 | −2.067 | 24.541 | 13.371 | 1.00 | 17.08 | N |
| ATOM | 1984 | CA | GLN | L | 38 | −0.767 | 25.101 | 13.016 | 1.00 | 18.03 | C |
| ATOM | 1985 | CB | GLN | L | 38 | −0.328 | 26.161 | 14.033 | 1.00 | 17.60 | C |
| ATOM | 1986 | CG | GLN | L | 38 | 0.883 | 26.933 | 13.567 | 1.00 | 16.82 | C |
| ATOM | 1987 | CD | GLN | L | 38 | 1.436 | 27.872 | 14.613 | 1.00 | 16.59 | C |
| ATOM | 1988 | OE1 | GLN | L | 38 | 1.303 | 27.637 | 15.812 | 1.00 | 17.00 | O |
| ATOM | 1989 | NE2 | GLN | L | 38 | 2.074 | 28.945 | 14.159 | 1.00 | 15.60 | N |
| ATOM | 1990 | C | GLN | L | 38 | 0.313 | 24.031 | 12.910 | 1.00 | 18.74 | C |
| ATOM | 1991 | O | GLN | L | 38 | 0.629 | 23.359 | 13.881 | 1.00 | 18.81 | O |
| ATOM | 1992 | N | LYS | L | 39 | 0.870 | 23.904 | 11.714 | 1.00 | 20.47 | N |
| ATOM | 1993 | CA | LYS | L | 39 | 2.044 | 23.080 | 11.448 | 1.00 | 21.89 | C |
| ATOM | 1994 | CB | LYS | L | 39 | 1.969 | 22.561 | 10.024 | 1.00 | 22.13 | C |
| ATOM | 1995 | CG | LYS | L | 39 | 1.078 | 21.358 | 9.940 | 1.00 | 23.21 | C |
| ATOM | 1996 | CD | LYS | L | 39 | 0.293 | 21.331 | 8.674 | 1.00 | 25.41 | C |
| ATOM | 1997 | CE | LYS | L | 39 | −0.458 | 20.038 | 8.590 | 1.00 | 28.15 | C |
| ATOM | 1998 | NZ | LYS | L | 39 | −1.838 | 20.308 | 8.085 | 1.00 | 32.26 | N |
| ATOM | 1999 | C | LYS | L | 39 | 3.336 | 23.864 | 11.673 | 1.00 | 22.83 | C |
| ATOM | 2000 | O | LYS | L | 39 | 3.352 | 25.089 | 11.502 | 1.00 | 22.90 | O |
| ATOM | 2001 | N | PRO | L | 40 | 4.425 | 23.168 | 12.069 | 1.00 | 23.85 | N |
| ATOM | 2002 | CA | PRO | L | 40 | 5.637 | 23.868 | 12.538 | 1.00 | 24.27 | C |
| ATOM | 2003 | CB | PRO | L | 40 | 6.602 | 22.721 | 12.858 | 1.00 | 24.53 | C |
| ATOM | 2004 | CG | PRO | L | 40 | 5.705 | 21.521 | 13.092 | 1.00 | 23.88 | C |
| ATOM | 2005 | CD | PRO | L | 40 | 4.600 | 21.702 | 12.097 | 1.00 | 23.88 | C |
| ATOM | 2006 | C | PRO | L | 40 | 6.224 | 24.803 | 11.485 | 1.00 | 24.66 | C |
| ATOM | 2007 | O | PRO | L | 40 | 6.161 | 24.507 | 10.296 | 1.00 | 24.91 | O |
| ATOM | 2008 | N | GLY | L | 41 | 6.751 | 25.944 | 11.923 | 1.00 | 25.25 | N |
| ATOM | 2009 | CA | GLY | L | 41 | 7.295 | 26.957 | 11.010 | 1.00 | 25.33 | C |
| ATOM | 2010 | C | GLY | L | 41 | 6.268 | 27.619 | 10.090 | 1.00 | 25.55 | C |
| ATOM | 2011 | O | GLY | L | 41 | 6.624 | 28.446 | 9.247 | 1.00 | 25.83 | O |
| ATOM | 2012 | N | GLN | L | 42 | 4.996 | 27.252 | 10.245 | 1.00 | 25.25 | N |
| ATOM | 2013 | CA | GLN | L | 42 | 3.910 | 27.813 | 9.440 | 1.00 | 24.80 | C |
| ATOM | 2014 | CB | GLN | L | 42 | 3.136 | 26.699 | 8.720 | 1.00 | 25.04 | C |
| ATOM | 2015 | CG | GLN | L | 42 | 3.973 | 25.876 | 7.737 | 1.00 | 28.32 | C |
| ATOM | 2016 | CD | GLN | L | 42 | 3.125 | 24.929 | 6.847 | 1.00 | 33.13 | C |
| ATOM | 2017 | OE1 | GLN | L | 42 | 1.878 | 24.963 | 6.864 | 1.00 | 33.13 | O |
| ATOM | 2018 | NE2 | GLN | L | 42 | 3.816 | 24.074 | 6.071 | 1.00 | 31.64 | N |
| ATOM | 2019 | C | GLN | L | 42 | 2.967 | 28.602 | 10.333 | 1.00 | 23.82 | C |
| ATOM | 2020 | O | GLN | L | 42 | 3.068 | 28.560 | 11.566 | 1.00 | 23.19 | O |
| ATOM | 2021 | N | SER | L | 43 | 2.040 | 29.324 | 9.718 | 1.00 | 23.05 | N |
| ATOM | 2022 | CA | SER | L | 43 | 1.046 | 30.044 | 10.504 | 1.00 | 22.36 | C |
| ATOM | 2023 | CB | SER | L | 43 | 0.731 | 31.404 | 9.883 | 1.00 | 22.02 | C |
| ATOM | 2024 | OG | SER | L | 43 | 0.471 | 31.278 | 8.506 | 1.00 | 23.11 | O |
| ATOM | 2025 | C | SER | L | 43 | −0.193 | 29.174 | 10.667 | 1.00 | 21.55 | C |
| ATOM | 2026 | O | SER | L | 43 | −0.365 | 28.214 | 9.926 | 1.00 | 21.40 | O |
| ATOM | 2027 | N | PRO | L | 44 | −1.053 | 29.489 | 11.650 | 1.00 | 21.24 | N |
| ATOM | 2028 | CA | PRO | L | 44 | −2.249 | 28.659 | 11.832 | 1.00 | 21.01 | C |
| ATOM | 2029 | CB | PRO | L | 44 | −2.948 | 29.301 | 13.038 | 1.00 | 21.03 | C |
| ATOM | 2030 | CG | PRO | L | 44 | −1.915 | 30.167 | 13.688 | 1.00 | 21.14 | C |
| ATOM | 2031 | CD | PRO | L | 44 | −1.042 | 30.636 | 12.573 | 1.00 | 21.10 | C |
| ATOM | 2032 | C | PRO | L | 44 | −3.166 | 28.735 | 10.613 | 1.00 | 20.79 | C |
| ATOM | 2033 | O | PRO | L | 44 | −3.190 | 29.757 | 9.912 | 1.00 | 20.74 | O |
| ATOM | 2034 | N | LYS | L | 45 | −3.895 | 27.653 | 10.359 | 1.00 | 20.28 | N |
| ATOM | 2035 | CA | LYS | L | 45 | −4.926 | 27.628 | 9.315 | 1.00 | 19.58 | C |
| ATOM | 2036 | CB | LYS | L | 45 | −4.629 | 26.538 | 8.293 | 1.00 | 19.53 | C |
| ATOM | 2037 | CG | LYS | L | 45 | −3.487 | 26.836 | 7.397 | 1.00 | 22.03 | C |
| ATOM | 2038 | CD | LYS | L | 45 | −3.123 | 25.618 | 6.540 | 1.00 | 26.74 | C |
| ATOM | 2039 | CE | LYS | L | 45 | −1.890 | 25.915 | 5.693 | 1.00 | 28.44 | C |
| ATOM | 2040 | NZ | LYS | L | 45 | −2.087 | 27.211 | 4.939 | 1.00 | 30.30 | N |
| ATOM | 2041 | C | LYS | L | 45 | −6.286 | 27.358 | 9.942 | 1.00 | 18.37 | C |
| ATOM | 2042 | O | LYS | L | 45 | −6.420 | 26.445 | 10.754 | 1.00 | 18.05 | O |
| ATOM | 2043 | N | LEU | L | 46 | −7.279 | 28.151 | 9.558 | 1.00 | 17.28 | N |
| ATOM | 2044 | CA | LEU | L | 46 | −8.659 | 27.975 | 10.008 | 1.00 | 16.60 | C |
| ATOM | 2045 | CB | LEU | L | 46 | −9.531 | 29.127 | 9.497 | 1.00 | 16.45 | C |
| ATOM | 2046 | CG | LEU | L | 46 | −11.025 | 29.178 | 9.827 | 1.00 | 15.80 | C |
| ATOM | 2047 | CD1 | LEU | L | 46 | −11.281 | 29.072 | 11.355 | 1.00 | 14.64 | C |
| ATOM | 2048 | CD2 | LEU | L | 46 | −11.591 | 30.468 | 9.271 | 1.00 | 13.92 | C |
| ATOM | 2049 | C | LEU | L | 46 | −9.258 | 26.649 | 9.565 | 1.00 | 16.21 | C |
| ATOM | 2050 | O | LEU | L | 46 | −9.240 | 26.307 | 8.387 | 1.00 | 16.50 | O |
| ATOM | 2051 | N | LEU | L | 47 | −9.789 | 25.899 | 10.515 | 1.00 | 16.05 | N |
| ATOM | 2052 | CA | LEU | L | 47 | −10.431 | 24.632 | 10.185 | 1.00 | 15.97 | C |
| ATOM | 2053 | CB | LEU | L | 47 | −9.932 | 23.524 | 11.101 | 1.00 | 15.64 | C |
| ATOM | 2054 | CG | LEU | L | 47 | −8.461 | 23.135 | 11.053 | 1.00 | 16.41 | C |
| ATOM | 2055 | CD1 | LEU | L | 47 | −8.216 | 22.069 | 12.141 | 1.00 | 17.67 | C |
| ATOM | 2056 | CD2 | LEU | L | 47 | −8.019 | 22.617 | 9.665 | 1.00 | 14.95 | C |
| ATOM | 2057 | C | LEU | L | 47 | −11.951 | 24.742 | 10.272 | 1.00 | 15.72 | C |
| ATOM | 2058 | O | LEU | L | 47 | −12.670 | 24.280 | 9.379 | 1.00 | 14.99 | O |
| ATOM | 2059 | N | ILE | L | 48 | −12.421 | 25.374 | 11.346 | 1.00 | 15.38 | N |
| ATOM | 2060 | CA | ILE | L | 48 | −13.840 | 25.418 | 11.670 | 1.00 | 15.90 | C |
| ATOM | 2061 | CB | ILE | L | 48 | −14.202 | 24.251 | 12.626 | 1.00 | 15.97 | C |
| ATOM | 2062 | CG1 | ILE | L | 48 | −14.220 | 22.926 | 11.863 | 1.00 | 16.43 | C |
| ATOM | 2063 | CD1 | ILE | L | 48 | −14.465 | 21.720 | 12.742 | 1.00 | 19.72 | C |
| ATOM | 2064 | CG2 | ILE | L | 48 | −15.528 | 24.505 | 13.312 | 1.00 | 16.23 | C |
| ATOM | 2065 | C | ILE | L | 48 | −14.183 | 26.746 | 12.345 | 1.00 | 15.78 | C |
| ATOM | 2066 | O | ILE | L | 48 | −13.471 | 27.175 | 13.259 | 1.00 | 16.31 | O |
| ATOM | 2067 | N | TYR | L | 49 | −15.264 | 27.383 | 11.913 | 1.00 | 15.23 | N |
| ATOM | 2068 | CA | TYR | L | 49 | −15.700 | 28.645 | 12.516 | 1.00 | 15.46 | C |
| ATOM | 2069 | CB | TYR | L | 49 | −15.626 | 29.815 | 11.499 | 1.00 | 15.36 | C |
| ATOM | 2070 | CG | TYR | L | 49 | −16.551 | 29.698 | 10.303 | 1.00 | 14.85 | C |
| ATOM | 2071 | CD1 | TYR | L | 49 | −17.885 | 30.086 | 10.385 | 1.00 | 15.57 | C |
| ATOM | 2072 | CE1 | TYR | L | 49 | −18.743 | 29.973 | 9.281 | 1.00 | 15.92 | C |
| ATOM | 2073 | CZ | TYR | L | 49 | −18.263 | 29.463 | 8.084 | 1.00 | 16.35 | C |
| ATOM | 2074 | OH | TYR | L | 49 | −19.106 | 29.345 | 7.008 | 1.00 | 16.91 | O |
| ATOM | 2075 | CE2 | TYR | L | 49 | −16.950 | 29.068 | 7.972 | 1.00 | 15.17 | C |
| ATOM | 2076 | CD2 | TYR | L | 49 | −16.093 | 29.195 | 9.088 | 1.00 | 15.91 | C |
| ATOM | 2077 | C | TYR | L | 49 | −17.108 | 28.506 | 13.097 | 1.00 | 15.42 | C |
| ATOM | 2078 | O | TYR | L | 49 | −17.884 | 27.669 | 12.633 | 1.00 | 15.47 | O |
| ATOM | 2079 | N | LYS | L | 50 | −17.433 | 29.337 | 14.089 | 1.00 | 15.38 | N |
| ATOM | 2080 | CA | LYS | L | 50 | −18.726 | 29.284 | 14.785 | 1.00 | 15.71 | C |
| ATOM | 2081 | CB | LYS | L | 50 | −19.848 | 29.952 | 13.974 | 1.00 | 16.12 | C |
| ATOM | 2082 | CG | LYS | L | 50 | −19.871 | 31.485 | 14.047 | 1.00 | 15.84 | C |
| ATOM | 2083 | CD | LYS | L | 50 | −21.082 | 32.043 | 13.322 | 1.00 | 16.83 | C |
| ATOM | 2084 | CE | LYS | L | 50 | −21.068 | 31.687 | 11.842 | 1.00 | 17.42 | C |
| ATOM | 2085 | NZ | LYS | L | 50 | −22.240 | 32.230 | 11.108 | 1.00 | 17.18 | N |
| ATOM | 2086 | C | LYS | L | 50 | −19.128 | 27.860 | 15.134 | 1.00 | 15.95 | C |
| ATOM | 2087 | O | LYS | L | 50 | −20.167 | 27.365 | 14.685 | 1.00 | 15.68 | O |
| ATOM | 2088 | N | VAL | L | 51 | −18.267 | 27.201 | 15.908 | 1.00 | 15.77 | N |
| ATOM | 2089 | CA | VAL | L | 51 | −18.538 | 25.888 | 16.489 | 1.00 | 15.36 | C |
| ATOM | 2090 | CB | VAL | L | 51 | −19.925 | 25.816 | 17.185 | 1.00 | 14.77 | C |
| ATOM | 2091 | CG1 | VAL | L | 51 | −20.081 | 24.486 | 17.877 | 1.00 | 13.38 | C |
| ATOM | 2092 | CG2 | VAL | L | 51 | −20.088 | 26.955 | 18.140 | 1.00 | 13.98 | C |
| ATOM | 2093 | C | VAL | L | 51 | −18.437 | 24.728 | 15.508 | 1.00 | 15.94 | C |
| ATOM | 2094 | O | VAL | L | 51 | −17.696 | 23.772 | 15.766 | 1.00 | 15.63 | O |
| ATOM | 2095 | N | SER | L | 52 | −19.198 | 24.791 | 14.409 | 1.00 | 16.05 | N |
| ATOM | 2096 | CA | SER | L | 52 | −19.420 | 23.589 | 13.582 | 1.00 | 16.06 | C |
| ATOM | 2097 | CB | SER | L | 52 | −20.816 | 23.042 | 13.861 | 1.00 | 16.26 | C |
| ATOM | 2098 | OG | SER | L | 52 | −21.798 | 23.940 | 13.381 | 1.00 | 17.01 | O |
| ATOM | 2099 | C | SER | L | 52 | −19.255 | 23.730 | 12.064 | 1.00 | 16.06 | C |
| ATOM | 2100 | O | SER | L | 52 | −19.430 | 22.748 | 11.331 | 1.00 | 15.89 | O |
| ATOM | 2101 | N | ASN | L | 53 | −18.942 | 24.931 | 11.588 | 1.00 | 16.17 | N |
| ATOM | 2102 | CA | ASN | L | 53 | −18.901 | 25.189 | 10.146 | 1.00 | 16.89 | C |
| ATOM | 2103 | CB | ASN | L | 53 | −19.295 | 26.631 | 9.866 | 1.00 | 17.11 | C |
| ATOM | 2104 | CG | ASN | L | 53 | −20.693 | 26.946 | 10.335 | 1.00 | 17.04 | C |
| ATOM | 2105 | OD1 | ASN | L | 53 | −20.885 | 27.470 | 11.438 | 1.00 | 17.25 | O |
| ATOM | 2106 | ND2 | ASN | L | 53 | −21.681 | 26.604 | 9.516 | 1.00 | 15.41 | N |
| ATOM | 2107 | C | ASN | L | 53 | −17.535 | 24.916 | 9.561 | 1.00 | 16.95 | C |
| ATOM | 2108 | O | ASN | L | 53 | −16.548 | 25.447 | 10.047 | 1.00 | 18.12 | O |
| ATOM | 2109 | N | LEU | L | 54 | −17.466 | 24.080 | 8.535 | 1.00 | 16.96 | N |
| ATOM | 2110 | CA | LEU | L | 54 | −16.181 | 23.732 | 7.927 | 1.00 | 16.91 | C |
| ATOM | 2111 | CB | LEU | L | 54 | −16.268 | 22.432 | 7.127 | 1.00 | 16.87 | C |
| ATOM | 2112 | CG | LEU | L | 54 | −16.318 | 21.110 | 7.895 | 1.00 | 18.12 | C |
| ATOM | 2113 | CD1 | LEU | L | 54 | −16.263 | 19.931 | 6.913 | 1.00 | 19.34 | C |
| ATOM | 2114 | CD2 | LEU | L | 54 | −15.158 | 21.022 | 8.853 | 1.00 | 18.81 | C |
| ATOM | 2115 | C | LEU | L | 54 | −15.734 | 24.844 | 7.019 | 1.00 | 16.89 | C |
| ATOM | 2116 | O | LEU | L | 54 | −16.503 | 25.313 | 6.176 | 1.00 | 16.65 | O |
| ATOM | 2117 | N | PHE | L | 55 | −14.494 | 25.278 | 7.209 | 1.00 | 17.02 | N |
| ATOM | 2118 | CA | PHE | L | 55 | −13.886 | 26.238 | 6.313 | 1.00 | 17.07 | C |
| ATOM | 2119 | CB | PHE | L | 55 | −12.580 | 26.786 | 6.883 | 1.00 | 16.46 | C |
| ATOM | 2120 | CG | PHE | L | 55 | −12.023 | 27.980 | 6.124 | 1.00 | 15.09 | C |
| ATOM | 2121 | CD1 | PHE | L | 55 | −12.765 | 29.154 | 5.989 | 1.00 | 14.18 | C |
| ATOM | 2122 | CE1 | PHE | L | 55 | −12.230 | 30.275 | 5.286 | 1.00 | 14.65 | C |
| ATOM | 2123 | CZ | PHE | L | 55 | −10.949 | 30.201 | 4.742 | 1.00 | 13.81 | C |
| ATOM | 2124 | CE2 | PHE | L | 55 | −10.206 | 29.026 | 4.888 | 1.00 | 12.98 | C |
| ATOM | 2125 | CD2 | PHE | L | 55 | −10.737 | 27.939 | 5.586 | 1.00 | 12.41 | C |
| ATOM | 2126 | C | PHE | L | 55 | −13.645 | 25.535 | 4.990 | 1.00 | 17.86 | C |
| ATOM | 2127 | O | PHE | L | 55 | −13.359 | 24.323 | 4.963 | 1.00 | 18.04 | O |
| ATOM | 2128 | N | SER | L | 56 | −13.783 | 26.305 | 3.909 | 1.00 | 18.01 | N |
| ATOM | 2129 | CA | SER | L | 56 | −13.685 | 25.814 | 2.546 | 1.00 | 18.17 | C |
| ATOM | 2130 | CB | SER | L | 56 | −13.766 | 26.979 | 1.569 | 1.00 | 18.00 | C |
| ATOM | 2131 | OG | SER | L | 56 | −13.355 | 26.558 | 0.293 | 1.00 | 19.23 | O |
| ATOM | 2132 | C | SER | L | 56 | −12.385 | 25.085 | 2.356 | 1.00 | 18.14 | C |
| ATOM | 2133 | O | SER | L | 56 | −11.341 | 25.567 | 2.785 | 1.00 | 18.44 | O |
| ATOM | 2134 | N | GLY | L | 57 | −12.456 | 23.908 | 1.738 | 1.00 | 18.37 | N |
| ATOM | 2135 | CA | GLY | L | 57 | −11.269 | 23.070 | 1.524 | 1.00 | 18.34 | C |
| ATOM | 2136 | C | GLY | L | 57 | −11.020 | 22.056 | 2.629 | 1.00 | 18.69 | C |
| ATOM | 2137 | O | GLY | L | 57 | −10.252 | 21.101 | 2.445 | 1.00 | 19.56 | O |
| ATOM | 2138 | N | VAL | L | 58 | −11.666 | 22.240 | 3.776 | 1.00 | 18.40 | N |
| ATOM | 2139 | CA | VAL | L | 58 | −11.445 | 21.348 | 4.910 | 1.00 | 18.31 | C |
| ATOM | 2140 | CB | VAL | L | 58 | −11.704 | 22.071 | 6.233 | 1.00 | 18.15 | C |
| ATOM | 2141 | CG1 | VAL | L | 58 | −11.547 | 21.130 | 7.409 | 1.00 | 17.42 | C |
| ATOM | 2142 | CG2 | VAL | L | 58 | −10.755 | 23.273 | 6.372 | 1.00 | 18.05 | C |
| ATOM | 2143 | C | VAL | L | 58 | −12.322 | 20.101 | 4.778 | 1.00 | 18.62 | C |
| ATOM | 2144 | O | VAL | L | 58 | −13.535 | 20.217 | 4.648 | 1.00 | 18.50 | O |
| ATOM | 2145 | N | PRO | L | 59 | −11.705 | 18.902 | 4.799 | 1.00 | 18.80 | N |
| ATOM | 2146 | CA | PRO | L | 59 | −12.470 | 17.661 | 4.592 | 1.00 | 18.73 | C |
| ATOM | 2147 | CB | PRO | L | 59 | −11.375 | 16.590 | 4.483 | 1.00 | 18.80 | C |
| ATOM | 2148 | CG | PRO | L | 59 | −10.186 | 17.175 | 5.232 | 1.00 | 18.21 | C |
| ATOM | 2149 | CD | PRO | L | 59 | −10.257 | 18.650 | 4.972 | 1.00 | 18.41 | C |
| ATOM | 2150 | C | PRO | L | 59 | −13.451 | 17.327 | 5.740 | 1.00 | 19.24 | C |
| ATOM | 2151 | O | PRO | L | 59 | −13.165 | 17.606 | 6.905 | 1.00 | 18.95 | O |
| ATOM | 2152 | N | ASP | L | 60 | −14.574 | 16.697 | 5.389 | 1.00 | 19.73 | N |
| ATOM | 2153 | CA | ASP | L | 60 | −15.670 | 16.400 | 6.316 | 1.00 | 20.74 | C |
| ATOM | 2154 | CB | ASP | L | 60 | −16.947 | 16.020 | 5.529 | 1.00 | 20.90 | C |
| ATOM | 2155 | CG | ASP | L | 60 | −16.814 | 14.695 | 4.748 | 1.00 | 22.46 | C |
| ATOM | 2156 | OD1 | ASP | L | 60 | −15.711 | 14.126 | 4.639 | 1.00 | 22.94 | O |
| ATOM | 2157 | OD2 | ASP | L | 60 | −17.844 | 14.210 | 4.229 | 1.00 | 26.43 | O |
| ATOM | 2158 | C | ASP | L | 60 | −15.369 | 15.358 | 7.422 | 1.00 | 20.90 | C |
| ATOM | 2159 | O | ASP | L | 60 | −16.255 | 14.984 | 8.196 | 1.00 | 21.21 | O |
| ATOM | 2160 | N | ARG | L | 61 | −14.136 | 14.880 | 7.499 | 1.00 | 20.80 | N |
| ATOM | 2161 | CA | ARG | L | 61 | −13.773 | 14.025 | 8.621 | 1.00 | 21.24 | C |
| ATOM | 2162 | CB | ARG | L | 61 | −12.603 | 13.097 | 8.272 | 1.00 | 21.24 | C |
| ATOM | 2163 | CG | ARG | L | 61 | −11.340 | 13.812 | 7.986 | 1.00 | 23.21 | C |
| ATOM | 2164 | CD | ARG | L | 61 | −10.204 | 12.831 | 7.853 | 1.00 | 27.24 | C |
| ATOM | 2165 | NE | ARG | L | 61 | −8.940 | 13.499 | 8.127 | 1.00 | 29.56 | N |
| ATOM | 2166 | CZ | ARG | L | 61 | −8.212 | 14.089 | 7.189 | 1.00 | 31.82 | C |
| ATOM | 2167 | NH1 | ARG | L | 61 | −8.606 | 14.071 | 5.924 | 1.00 | 34.08 | N |
| ATOM | 2168 | NH2 | ARG | L | 61 | −7.088 | 14.685 | 7.507 | 1.00 | 33.02 | N |
| ATOM | 2169 | C | ARG | L | 61 | −13.500 | 14.859 | 9.880 | 1.00 | 20.67 | C |
| ATOM | 2170 | O | ARG | L | 61 | −13.367 | 14.322 | 10.980 | 1.00 | 20.85 | O |
| ATOM | 2171 | N | PHE | L | 62 | −13.437 | 16.174 | 9.687 | 1.00 | 20.07 | N |
| ATOM | 2172 | CA | PHE | L | 62 | −13.407 | 17.151 | 10.756 | 1.00 | 19.44 | C |
| ATOM | 2173 | CB | PHE | L | 62 | −12.603 | 18.374 | 10.328 | 1.00 | 19.45 | C |
| ATOM | 2174 | CG | PHE | L | 62 | −11.129 | 18.173 | 10.390 | 1.00 | 19.64 | C |
| ATOM | 2175 | CD1 | PHE | L | 62 | −10.433 | 18.415 | 11.573 | 1.00 | 20.46 | C |
| ATOM | 2176 | CE1 | PHE | L | 62 | −9.059 | 18.239 | 11.647 | 1.00 | 21.37 | C |
| ATOM | 2177 | CZ | PHE | L | 62 | −8.361 | 17.787 | 10.536 | 1.00 | 20.97 | C |
| ATOM | 2178 | CE2 | PHE | L | 62 | −9.058 | 17.533 | 9.339 | 1.00 | 21.87 | C |
| ATOM | 2179 | CD2 | PHE | L | 62 | −10.434 | 17.729 | 9.279 | 1.00 | 19.86 | C |
| ATOM | 2180 | C | PHE | L | 62 | −14.813 | 17.591 | 11.086 | 1.00 | 19.16 | C |
| ATOM | 2181 | O | PHE | L | 62 | −15.599 | 17.932 | 10.200 | 1.00 | 19.56 | O |
| ATOM | 2182 | N | SER | L | 63 | −15.137 | 17.571 | 12.370 | 1.00 | 18.50 | N |
| ATOM | 2183 | CA | SER | L | 63 | −16.408 | 18.086 | 12.833 | 1.00 | 17.79 | C |
| ATOM | 2184 | CB | SER | L | 63 | −17.436 | 16.969 | 12.961 | 1.00 | 17.30 | C |
| ATOM | 2185 | OG | SER | L | 63 | −17.020 | 16.033 | 13.934 | 1.00 | 19.56 | O |
| ATOM | 2186 | C | SER | L | 63 | −16.198 | 18.792 | 14.157 | 1.00 | 17.17 | C |
| ATOM | 2187 | O | SER | L | 63 | −15.291 | 18.445 | 14.929 | 1.00 | 17.03 | O |
| ATOM | 2188 | N | GLY | L | 64 | −17.032 | 19.796 | 14.403 | 1.00 | 16.69 | N |
| ATOM | 2189 | CA | GLY | L | 64 | −16.962 | 20.563 | 15.628 | 1.00 | 16.49 | C |
| ATOM | 2190 | C | GLY | L | 64 | −18.322 | 20.577 | 16.262 | 1.00 | 16.51 | C |
| ATOM | 2191 | O | GLY | L | 64 | −19.333 | 20.494 | 15.557 | 1.00 | 16.50 | O |
| ATOM | 2192 | N | SER | L | 65 | −18.354 | 20.681 | 17.590 | 1.00 | 16.25 | N |
| ATOM | 2193 | CA | SER | L | 65 | −19.611 | 20.625 | 18.332 | 1.00 | 16.40 | C |
| ATOM | 2194 | CB | SER | L | 65 | −20.024 | 19.171 | 18.573 | 1.00 | 16.31 | C |
| ATOM | 2195 | OG | SER | L | 65 | −19.203 | 18.605 | 19.589 | 1.00 | 18.77 | O |
| ATOM | 2196 | C | SER | L | 65 | −19.453 | 21.345 | 19.660 | 1.00 | 16.20 | C |
| ATOM | 2197 | O | SER | L | 65 | −18.385 | 21.879 | 19.954 | 1.00 | 16.28 | O |
| ATOM | 2198 | N | GLY | L | 66 | −20.516 | 21.351 | 20.460 | 1.00 | 16.29 | N |
| ATOM | 2199 | CA | GLY | L | 66 | −20.505 | 22.022 | 21.751 | 1.00 | 16.90 | C |
| ATOM | 2200 | C | GLY | L | 66 | −21.320 | 23.302 | 21.783 | 1.00 | 17.43 | C |
| ATOM | 2201 | O | GLY | L | 66 | −21.970 | 23.658 | 20.805 | 1.00 | 17.44 | O |
| ATOM | 2202 | N | SER | L | 67 | −21.257 | 24.013 | 22.904 | 1.00 | 17.87 | N |
| ATOM | 2203 | CA | SER | L | 67 | −22.187 | 25.091 | 23.172 | 1.00 | 18.46 | C |
| ATOM | 2204 | CB | SER | L | 67 | −23.614 | 24.524 | 23.134 | 1.00 | 18.41 | C |
| ATOM | 2205 | OG | SER | L | 67 | −24.497 | 25.211 | 23.986 | 1.00 | 20.37 | O |
| ATOM | 2206 | C | SER | L | 67 | −21.870 | 25.797 | 24.496 | 1.00 | 18.90 | C |
| ATOM | 2207 | O | SER | L | 67 | −21.383 | 25.173 | 25.448 | 1.00 | 18.91 | O |
| ATOM | 2208 | N | GLY | L | 68 | −22.122 | 27.107 | 24.532 | 1.00 | 19.43 | N |
| ATOM | 2209 | CA | GLY | L | 68 | −21.931 | 27.931 | 25.729 | 1.00 | 19.64 | C |
| ATOM | 2210 | C | GLY | L | 68 | −20.466 | 28.080 | 26.095 | 1.00 | 20.38 | C |
| ATOM | 2211 | O | GLY | L | 68 | −19.799 | 28.984 | 25.608 | 1.00 | 20.60 | O |
| ATOM | 2212 | N | THR | L | 69 | −19.969 | 27.176 | 26.942 | 1.00 | 20.81 | N |
| ATOM | 2213 | CA | THR | L | 69 | −18.593 | 27.208 | 27.437 | 1.00 | 21.24 | C |
| ATOM | 2214 | CB | THR | L | 69 | −18.538 | 27.353 | 28.976 | 1.00 | 21.43 | C |
| ATOM | 2215 | OG1 | THR | L | 69 | −19.283 | 26.286 | 29.593 | 1.00 | 22.39 | O |
| ATOM | 2216 | CG2 | THR | L | 69 | −19.091 | 28.705 | 29.421 | 1.00 | 20.98 | C |
| ATOM | 2217 | C | THR | L | 69 | −17.817 | 25.944 | 27.107 | 1.00 | 21.50 | C |
| ATOM | 2218 | O | THR | L | 69 | −16.661 | 25.813 | 27.495 | 1.00 | 21.93 | O |
| ATOM | 2219 | N | ASP | L | 70 | −18.444 | 25.016 | 26.396 | 1.00 | 21.76 | N |
| ATOM | 2220 | CA | ASP | L | 70 | −17.883 | 23.689 | 26.246 | 1.00 | 21.89 | C |
| ATOM | 2221 | CB | ASP | L | 70 | −18.645 | 22.696 | 27.133 | 1.00 | 22.65 | C |
| ATOM | 2222 | CG | ASP | L | 70 | −17.948 | 21.342 | 27.228 | 1.00 | 25.91 | C |
| ATOM | 2223 | OD1 | ASP | L | 70 | −17.110 | 21.170 | 28.143 | 1.00 | 29.08 | O |
| ATOM | 2224 | OD2 | ASP | L | 70 | −18.225 | 20.455 | 26.382 | 1.00 | 29.31 | O |
| ATOM | 2225 | C | ASP | L | 70 | −17.867 | 23.250 | 24.788 | 1.00 | 21.18 | C |
| ATOM | 2226 | O | ASP | L | 70 | −18.910 | 23.141 | 24.141 | 1.00 | 21.16 | O |
| ATOM | 2227 | N | PHE | L | 71 | −16.668 | 23.013 | 24.262 | 1.00 | 20.20 | N |
| ATOM | 2228 | CA | PHE | L | 71 | −16.521 | 22.778 | 22.832 | 1.00 | 19.16 | C |
| ATOM | 2229 | CB | PHE | L | 71 | −16.051 | 24.053 | 22.125 | 1.00 | 18.77 | C |
| ATOM | 2230 | CG | PHE | L | 71 | −16.948 | 25.220 | 22.382 | 1.00 | 17.49 | C |
| ATOM | 2231 | CD1 | PHE | L | 71 | −18.120 | 25.381 | 21.658 | 1.00 | 17.47 | C |
| ATOM | 2232 | CE1 | PHE | L | 71 | −18.977 | 26.434 | 21.923 | 1.00 | 16.06 | C |
| ATOM | 2233 | CZ | PHE | L | 71 | −18.671 | 27.320 | 22.931 | 1.00 | 15.48 | C |
| ATOM | 2234 | CE2 | PHE | L | 71 | −17.516 | 27.161 | 23.663 | 1.00 | 15.29 | C |
| ATOM | 2235 | CD2 | PHE | L | 71 | −16.663 | 26.118 | 23.395 | 1.00 | 15.52 | C |
| ATOM | 2236 | C | PHE | L | 71 | −15.610 | 21.611 | 22.574 | 1.00 | 18.92 | C |
| ATOM | 2237 | O | PHE | L | 71 | −14.696 | 21.339 | 23.361 | 1.00 | 18.79 | O |
| ATOM | 2238 | N | THR | L | 72 | −15.893 | 20.908 | 21.486 | 1.00 | 18.53 | N |
| ATOM | 2239 | CA | THR | L | 72 | −15.147 | 19.714 | 21.124 | 1.00 | 18.83 | C |
| ATOM | 2240 | CB | THR | L | 72 | −15.893 | 18.409 | 21.575 | 1.00 | 18.80 | C |
| ATOM | 2241 | OG1 | THR | L | 72 | −16.034 | 18.393 | 23.005 | 1.00 | 18.46 | O |
| ATOM | 2242 | CG2 | THR | L | 72 | −15.117 | 17.177 | 21.176 | 1.00 | 18.87 | C |
| ATOM | 2243 | C | THR | L | 72 | −14.847 | 19.696 | 19.620 | 1.00 | 18.68 | C |
| ATOM | 2244 | O | THR | L | 72 | −15.712 | 19.980 | 18.802 | 1.00 | 18.97 | O |
| ATOM | 2245 | N | LEU | L | 73 | −13.604 | 19.387 | 19.279 | 1.00 | 18.99 | N |
| ATOM | 2246 | CA | LEU | L | 73 | −13.208 | 19.114 | 17.902 | 1.00 | 19.58 | C |
| ATOM | 2247 | CB | LEU | L | 73 | −11.980 | 19.945 | 17.513 | 1.00 | 19.12 | C |
| ATOM | 2248 | CG | LEU | L | 73 | −11.276 | 19.672 | 16.186 | 1.00 | 20.21 | C |
| ATOM | 2249 | CD1 | LEU | L | 73 | −12.236 | 19.723 | 15.029 | 1.00 | 23.90 | C |
| ATOM | 2250 | CD2 | LEU | L | 73 | −10.200 | 20.705 | 15.948 | 1.00 | 21.69 | C |
| ATOM | 2251 | C | LEU | L | 73 | −12.944 | 17.617 | 17.728 | 1.00 | 19.98 | C |
| ATOM | 2252 | O | LEU | L | 73 | −12.183 | 17.013 | 18.489 | 1.00 | 19.82 | O |
| ATOM | 2253 | N | LYS | L | 74 | −13.582 | 17.027 | 16.725 | 1.00 | 20.46 | N |
| ATOM | 2254 | CA | LYS | L | 74 | −13.415 | 15.616 | 16.446 | 1.00 | 21.14 | C |
| ATOM | 2255 | CB | LYS | L | 74 | −14.741 | 14.874 | 16.630 | 1.00 | 21.57 | C |
| ATOM | 2256 | CG | LYS | L | 74 | −15.228 | 14.827 | 18.075 | 1.00 | 23.80 | C |
| ATOM | 2257 | CD | LYS | L | 74 | −16.670 | 14.280 | 18.189 | 1.00 | 27.42 | C |
| ATOM | 2258 | CE | LYS | L | 74 | −17.162 | 14.258 | 19.650 | 1.00 | 28.92 | C |
| ATOM | 2259 | NZ | LYS | L | 74 | −16.246 | 13.476 | 20.572 | 1.00 | 29.05 | N |
| ATOM | 2260 | C | LYS | L | 74 | −12.887 | 15.388 | 15.037 | 1.00 | 21.28 | C |
| ATOM | 2261 | O | LYS | L | 74 | −13.246 | 16.102 | 14.110 | 1.00 | 20.80 | O |
| ATOM | 2262 | N | ILE | L | 75 | −12.020 | 14.391 | 14.904 | 1.00 | 21.88 | N |
| ATOM | 2263 | CA | ILE | L | 75 | −11.565 | 13.888 | 13.609 | 1.00 | 22.65 | C |
| ATOM | 2264 | CB | ILE | L | 75 | −10.019 | 13.971 | 13.478 | 1.00 | 22.23 | C |
| ATOM | 2265 | CG1 | ILE | L | 75 | −9.511 | 15.335 | 13.956 | 1.00 | 21.17 | C |
| ATOM | 2266 | CD1 | ILE | L | 75 | −8.006 | 15.557 | 13.842 | 1.00 | 19.05 | C |
| ATOM | 2267 | CG2 | ILE | L | 75 | −9.580 | 13.699 | 12.048 | 1.00 | 22.51 | C |
| ATOM | 2268 | C | ILE | L | 75 | −12.032 | 12.427 | 13.531 | 1.00 | 23.61 | C |
| ATOM | 2269 | O | ILE | L | 75 | −11.669 | 11.622 | 14.384 | 1.00 | 24.47 | O |
| ATOM | 2270 | N | SER | L | 76 | −12.850 | 12.082 | 12.545 | 1.00 | 24.20 | N |
| ATOM | 2271 | CA | SER | L | 76 | −13.480 | 10.755 | 12.544 | 1.00 | 25.10 | C |
| ATOM | 2272 | CB | SER | L | 76 | −14.660 | 10.679 | 11.569 | 1.00 | 24.80 | C |
| ATOM | 2273 | OG | SER | L | 76 | −14.224 | 10.967 | 10.256 | 1.00 | 26.78 | O |
| ATOM | 2274 | C | SER | L | 76 | −12.476 | 9.642 | 12.285 | 1.00 | 25.48 | C |
| ATOM | 2275 | O | SER | L | 76 | −12.586 | 8.552 | 12.862 | 1.00 | 25.77 | O |
| ATOM | 2276 | N | ARG | L | 77 | −11.500 | 9.918 | 11.424 | 1.00 | 26.22 | N |
| ATOM | 2277 | CA | ARG | L | 77 | −10.381 | 9.009 | 11.188 | 1.00 | 27.10 | C |
| ATOM | 2278 | CB | ARG | L | 77 | −10.622 | 8.058 | 9.999 | 1.00 | 27.09 | C |
| ATOM | 2279 | CG | ARG | L | 77 | −11.781 | 7.056 | 10.174 | 1.00 | 29.82 | C |
| ATOM | 2280 | CD | ARG | L | 77 | −11.817 | 6.000 | 9.069 | 1.00 | 34.56 | C |
| ATOM | 2281 | NE | ARG | L | 77 | −10.701 | 5.055 | 9.189 | 1.00 | 39.19 | N |
| ATOM | 2282 | CZ | ARG | L | 77 | −10.185 | 4.333 | 8.190 | 1.00 | 40.80 | C |
| ATOM | 2283 | NH1 | ARG | L | 77 | −10.676 | 4.422 | 6.957 | 1.00 | 41.37 | N |
| ATOM | 2284 | NH2 | ARG | L | 77 | −9.167 | 3.513 | 8.432 | 1.00 | 41.36 | N |
| ATOM | 2285 | C | ARG | L | 77 | −9.182 | 9.894 | 10.935 | 1.00 | 27.29 | C |
| ATOM | 2286 | O | ARG | L | 77 | −9.171 | 10.699 | 10.003 | 1.00 | 27.28 | O |
| ATOM | 2287 | N | VAL | L | 78 | −8.186 | 9.766 | 11.799 | 1.00 | 27.54 | N |
| ATOM | 2288 | CA | VAL | L | 78 | −7.000 | 10.605 | 11.726 | 1.00 | 27.72 | C |
| ATOM | 2289 | CB | VAL | L | 78 | −6.222 | 10.555 | 13.061 | 1.00 | 27.43 | C |
| ATOM | 2290 | CG1 | VAL | L | 78 | −4.830 | 11.173 | 12.943 | 1.00 | 27.88 | C |
| ATOM | 2291 | CG2 | VAL | L | 78 | −7.008 | 11.251 | 14.140 | 1.00 | 27.19 | C |
| ATOM | 2292 | C | VAL | L | 78 | −6.126 | 10.179 | 10.542 | 1.00 | 28.11 | C |
| ATOM | 2293 | O | VAL | L | 78 | −6.184 | 9.031 | 10.100 | 1.00 | 28.62 | O |
| ATOM | 2294 | N | GLU | L | 79 | −5.349 | 11.121 | 10.013 | 1.00 | 28.06 | N |
| ATOM | 2295 | CA | GLU | L | 79 | −4.373 | 10.823 | 8.976 | 1.00 | 27.78 | C |
| ATOM | 2296 | CB | GLU | L | 79 | −4.843 | 11.357 | 7.640 | 1.00 | 27.85 | C |
| ATOM | 2297 | CG | GLU | L | 79 | −6.281 | 11.038 | 7.309 | 1.00 | 29.12 | C |
| ATOM | 2298 | CD | GLU | L | 79 | −6.614 | 11.337 | 5.878 | 1.00 | 30.41 | C |
| ATOM | 2299 | OE1 | GLU | L | 79 | −5.902 | 12.170 | 5.258 | 1.00 | 30.96 | O |
| ATOM | 2300 | OE2 | GLU | L | 79 | −7.581 | 10.728 | 5.372 | 1.00 | 31.15 | O |
| ATOM | 2301 | C | GLU | L | 79 | −3.069 | 11.479 | 9.353 | 1.00 | 27.49 | C |
| ATOM | 2302 | O | GLU | L | 79 | −3.036 | 12.317 | 10.250 | 1.00 | 27.80 | O |
| ATOM | 2303 | N | ALA | L | 80 | −1.999 | 11.094 | 8.668 | 1.00 | 27.25 | N |
| ATOM | 2304 | CA | ALA | L | 80 | −0.659 | 11.633 | 8.901 | 1.00 | 26.95 | C |
| ATOM | 2305 | CB | ALA | L | 80 | 0.327 | 10.962 | 7.955 | 1.00 | 26.90 | C |
| ATOM | 2306 | C | ALA | L | 80 | −0.581 | 13.157 | 8.748 | 1.00 | 26.81 | C |
| ATOM | 2307 | O | ALA | L | 80 | 0.241 | 13.818 | 9.390 | 1.00 | 26.86 | O |
| ATOM | 2308 | N | GLU | L | 81 | −1.443 | 13.693 | 7.885 | 1.00 | 26.38 | N |
| ATOM | 2309 | CA | GLU | L | 81 | −1.466 | 15.109 | 7.506 | 1.00 | 25.93 | C |
| ATOM | 2310 | CB | GLU | L | 81 | −2.270 | 15.276 | 6.203 | 1.00 | 26.61 | C |
| ATOM | 2311 | CG | GLU | L | 81 | −1.798 | 14.390 | 5.000 | 1.00 | 30.53 | C |
| ATOM | 2312 | CD | GLU | L | 81 | −2.126 | 12.889 | 5.140 | 1.00 | 34.76 | C |
| ATOM | 2313 | OE1 | GLU | L | 81 | −2.804 | 12.502 | 6.116 | 1.00 | 36.95 | O |
| ATOM | 2314 | OE2 | GLU | L | 81 | −1.694 | 12.088 | 4.276 | 1.00 | 36.10 | O |
| ATOM | 2315 | C | GLU | L | 81 | −2.086 | 15.987 | 8.600 | 1.00 | 24.69 | C |
| ATOM | 2316 | O | GLU | L | 81 | −1.975 | 17.214 | 8.554 | 1.00 | 24.26 | O |
| ATOM | 2317 | N | ASP | L | 82 | −2.747 | 15.351 | 9.570 | 1.00 | 22.98 | N |
| ATOM | 2318 | CA | ASP | L | 82 | −3.485 | 16.060 | 10.620 | 1.00 | 21.55 | C |
| ATOM | 2319 | CB | ASP | L | 82 | −4.641 | 15.196 | 11.113 | 1.00 | 21.68 | C |
| ATOM | 2320 | CG | ASP | L | 82 | −5.582 | 14.824 | 10.007 | 1.00 | 21.21 | C |
| ATOM | 2321 | OD1 | ASP | L | 82 | −5.647 | 15.582 | 9.019 | 1.00 | 20.02 | O |
| ATOM | 2322 | OD2 | ASP | L | 82 | −6.247 | 13.781 | 10.116 | 1.00 | 22.06 | O |
| ATOM | 2323 | C | ASP | L | 82 | −2.604 | 16.449 | 11.791 | 1.00 | 20.71 | C |
| ATOM | 2324 | O | ASP | L | 82 | −3.071 | 17.007 | 12.784 | 1.00 | 20.71 | O |
| ATOM | 2325 | N | VAL | L | 83 | −1.322 | 16.160 | 11.649 | 1.00 | 19.27 | N |
| ATOM | 2326 | CA | VAL | L | 83 | −0.359 | 16.351 | 12.694 | 1.00 | 18.78 | C |
| ATOM | 2327 | CB | VAL | L | 83 | 0.916 | 15.501 | 12.388 | 1.00 | 18.89 | C |
| ATOM | 2328 | CG1 | VAL | L | 83 | 2.195 | 16.216 | 12.762 | 1.00 | 20.00 | C |
| ATOM | 2329 | CG2 | VAL | L | 83 | 0.818 | 14.162 | 13.092 | 1.00 | 16.96 | C |
| ATOM | 2330 | C | VAL | L | 83 | −0.084 | 17.830 | 12.860 | 1.00 | 18.33 | C |
| ATOM | 2331 | O | VAL | L | 83 | −0.014 | 18.558 | 11.862 | 1.00 | 18.26 | O |
| ATOM | 2332 | N | GLY | L | 84 | 0.048 | 18.267 | 14.116 | 1.00 | 17.53 | N |
| ATOM | 2333 | CA | GLY | L | 84 | 0.377 | 19.667 | 14.434 | 1.00 | 16.84 | C |
| ATOM | 2334 | C | GLY | L | 84 | −0.313 | 20.160 | 15.692 | 1.00 | 16.56 | C |
| ATOM | 2335 | O | GLY | L | 84 | −0.855 | 19.347 | 16.451 | 1.00 | 17.30 | O |
| ATOM | 2336 | N | VAL | L | 85 | −0.315 | 21.481 | 15.912 | 1.00 | 15.82 | N |
| ATOM | 2337 | CA | VAL | L | 85 | −0.916 | 22.077 | 17.113 | 1.00 | 15.22 | C |
| ATOM | 2338 | CB | VAL | L | 85 | 0.000 | 23.160 | 17.765 | 1.00 | 15.10 | C |
| ATOM | 2339 | CG1 | VAL | L | 85 | −0.638 | 23.716 | 19.032 | 1.00 | 15.31 | C |
| ATOM | 2340 | CG2 | VAL | L | 85 | 1.344 | 22.586 | 18.136 | 1.00 | 14.39 | C |
| ATOM | 2341 | C | VAL | L | 85 | −2.309 | 22.665 | 16.802 | 1.00 | 15.38 | C |
| ATOM | 2342 | O | VAL | L | 85 | −2.454 | 23.478 | 15.882 | 1.00 | 15.40 | O |
| ATOM | 2343 | N | TYR | L | 86 | −3.319 | 22.243 | 17.567 | 1.00 | 14.70 | N |
| ATOM | 2344 | CA | TYR | L | 86 | −4.692 | 22.723 | 17.395 | 1.00 | 14.53 | C |
| ATOM | 2345 | CB | TYR | L | 86 | −5.714 | 21.580 | 17.527 | 1.00 | 14.49 | C |
| ATOM | 2346 | CG | TYR | L | 86 | −5.679 | 20.621 | 16.362 | 1.00 | 14.28 | C |
| ATOM | 2347 | CD1 | TYR | L | 86 | −4.694 | 19.637 | 16.280 | 1.00 | 15.40 | C |
| ATOM | 2348 | CE1 | TYR | L | 86 | −4.631 | 18.757 | 15.203 | 1.00 | 16.61 | C |
| ATOM | 2349 | CZ | TYR | L | 86 | −5.560 | 18.859 | 14.178 | 1.00 | 18.20 | C |
| ATOM | 2350 | OH | TYR | L | 86 | −5.481 | 17.984 | 13.113 | 1.00 | 18.87 | O |
| ATOM | 2351 | CE2 | TYR | L | 86 | −6.563 | 19.834 | 14.234 | 1.00 | 17.48 | C |
| ATOM | 2352 | CD2 | TYR | L | 86 | −6.611 | 20.710 | 15.332 | 1.00 | 15.66 | C |
| ATOM | 2353 | C | TYR | L | 86 | −4.971 | 23.799 | 18.424 | 1.00 | 14.28 | C |
| ATOM | 2354 | O | TYR | L | 86 | −4.676 | 23.615 | 19.599 | 1.00 | 13.78 | O |
| ATOM | 2355 | N | PHE | L | 87 | −5.507 | 24.929 | 17.967 | 1.00 | 13.71 | N |
| ATOM | 2356 | CA | PHE | L | 87 | −5.853 | 26.030 | 18.853 | 1.00 | 13.35 | C |
| ATOM | 2357 | CB | PHE | L | 87 | −5.104 | 27.312 | 18.458 | 1.00 | 12.67 | C |
| ATOM | 2358 | CG | PHE | L | 87 | −3.627 | 27.295 | 18.753 | 1.00 | 12.07 | C |
| ATOM | 2359 | CD1 | PHE | L | 87 | −3.153 | 27.536 | 20.049 | 1.00 | 9.84 | C |
| ATOM | 2360 | CE1 | PHE | L | 87 | −1.793 | 27.562 | 20.318 | 1.00 | 7.79 | C |
| ATOM | 2361 | CZ | PHE | L | 87 | −0.869 | 27.345 | 19.287 | 1.00 | 7.68 | C |
| ATOM | 2362 | CE2 | PHE | L | 87 | −1.314 | 27.140 | 17.995 | 1.00 | 8.30 | C |
| ATOM | 2363 | CD2 | PHE | L | 87 | −2.697 | 27.114 | 17.729 | 1.00 | 10.37 | C |
| ATOM | 2364 | C | PHE | L | 87 | −7.338 | 26.305 | 18.709 | 1.00 | 13.47 | C |
| ATOM | 2365 | O | PHE | L | 87 | −7.842 | 26.373 | 17.588 | 1.00 | 14.09 | O |
| ATOM | 2366 | N | CYS | L | 88 | −8.037 | 26.464 | 19.826 | 1.00 | 13.31 | N |
| ATOM | 2367 | CA | CYS | L | 88 | −9.321 | 27.122 | 19.782 | 1.00 | 13.74 | C |
| ATOM | 2368 | CB | CYS | L | 88 | −10.316 | 26.593 | 20.833 | 1.00 | 14.07 | C |
| ATOM | 2369 | SG | CYS | L | 88 | −9.782 | 26.628 | 22.534 | 1.00 | 15.52 | S |
| ATOM | 2370 | C | CYS | L | 88 | −9.121 | 28.627 | 19.910 | 1.00 | 13.67 | C |
| ATOM | 2371 | O | CYS | L | 88 | −8.136 | 29.103 | 20.498 | 1.00 | 12.55 | O |
| ATOM | 2372 | N | SER | L | 89 | −10.075 | 29.362 | 19.341 | 1.00 | 13.71 | N |
| ATOM | 2373 | CA | SER | L | 89 | −9.995 | 30.798 | 19.227 | 1.00 | 13.55 | C |
| ATOM | 2374 | CB | SER | L | 89 | −9.498 | 31.151 | 17.823 | 1.00 | 13.62 | C |
| ATOM | 2375 | OG | SER | L | 89 | −9.767 | 32.496 | 17.473 | 1.00 | 13.69 | O |
| ATOM | 2376 | C | SER | L | 89 | −11.394 | 31.305 | 19.423 | 1.00 | 13.68 | C |
| ATOM | 2377 | O | SER | L | 89 | −12.324 | 30.742 | 18.852 | 1.00 | 13.65 | O |
| ATOM | 2378 | N | GLN | L | 90 | −11.554 | 32.347 | 20.234 | 1.00 | 14.06 | N |
| ATOM | 2379 | CA | GLN | L | 90 | −12.856 | 33.025 | 20.360 | 1.00 | 14.72 | C |
| ATOM | 2380 | CB | GLN | L | 90 | −13.414 | 32.951 | 21.801 | 1.00 | 14.69 | C |
| ATOM | 2381 | CG | GLN | L | 90 | −12.691 | 33.798 | 22.857 | 1.00 | 15.51 | C |
| ATOM | 2382 | CD | GLN | L | 90 | −13.220 | 35.222 | 22.962 | 1.00 | 16.78 | C |
| ATOM | 2383 | OE1 | GLN | L | 90 | −14.308 | 35.554 | 22.453 | 1.00 | 18.61 | O |
| ATOM | 2384 | NE2 | GLN | L | 90 | −12.452 | 36.076 | 23.616 | 1.00 | 16.62 | N |
| ATOM | 2385 | C | GLN | L | 90 | −12.835 | 34.469 | 19.841 | 1.00 | 15.01 | C |
| ATOM | 2386 | O | GLN | L | 90 | −11.836 | 35.185 | 20.000 | 1.00 | 14.96 | O |
| ATOM | 2387 | N | SER | L | 91 | −13.941 | 34.885 | 19.223 | 1.00 | 15.30 | N |
| ATOM | 2388 | CA | SER | L | 91 | −14.094 | 36.260 | 18.748 | 1.00 | 15.53 | C |
| ATOM | 2389 | CB | SER | L | 91 | −13.862 | 36.353 | 17.238 | 1.00 | 15.45 | C |
| ATOM | 2390 | OG | SER | L | 91 | −14.692 | 35.461 | 16.514 | 1.00 | 15.13 | O |
| ATOM | 2391 | C | SER | L | 91 | −15.441 | 36.880 | 19.142 | 1.00 | 16.06 | C |
| ATOM | 2392 | O | SER | L | 91 | −15.933 | 37.797 | 18.470 | 1.00 | 16.02 | O |
| ATOM | 2393 | N | THR | L | 92 | −16.009 | 36.386 | 20.243 | 1.00 | 16.31 | N |
| ATOM | 2394 | CA | THR | L | 92 | −17.186 | 36.981 | 20.874 | 1.00 | 16.65 | C |
| ATOM | 2395 | CB | THR | L | 92 | −17.894 | 35.961 | 21.777 | 1.00 | 16.56 | C |
| ATOM | 2396 | OG1 | THR | L | 92 | −18.210 | 34.786 | 21.020 | 1.00 | 16.21 | O |
| ATOM | 2397 | CG2 | THR | L | 92 | −19.166 | 36.552 | 22.356 | 1.00 | 15.97 | C |
| ATOM | 2398 | C | THR | L | 92 | −16.827 | 38.200 | 21.726 | 1.00 | 17.41 | C |
| ATOM | 2399 | O | THR | L | 92 | −17.466 | 39.246 | 21.618 | 1.00 | 17.18 | O |
| ATOM | 2400 | N | HIS | L | 93 | −15.806 | 38.053 | 22.573 | 1.00 | 18.17 | N |
| ATOM | 2401 | CA | HIS | L | 93 | −15.428 | 39.093 | 23.530 | 1.00 | 18.98 | C |
| ATOM | 2402 | CB | HIS | L | 93 | −15.402 | 38.524 | 24.947 | 1.00 | 19.11 | C |
| ATOM | 2403 | CG | HIS | L | 93 | −16.679 | 37.879 | 25.376 | 1.00 | 20.20 | C |
| ATOM | 2404 | ND1 | HIS | L | 93 | −17.755 | 38.600 | 25.850 | 1.00 | 21.13 | N |
| ATOM | 2405 | CE1 | HIS | L | 93 | −18.732 | 37.767 | 26.163 | 1.00 | 21.83 | C |
| ATOM | 2406 | NE2 | HIS | L | 93 | −18.324 | 36.532 | 25.922 | 1.00 | 21.79 | N |
| ATOM | 2407 | CD2 | HIS | L | 93 | −17.042 | 36.574 | 25.435 | 1.00 | 21.78 | C |
| ATOM | 2408 | C | HIS | L | 93 | −14.049 | 39.697 | 23.262 | 1.00 | 19.13 | C |
| ATOM | 2409 | O | HIS | L | 93 | −13.093 | 38.973 | 22.972 | 1.00 | 19.87 | O |
| ATOM | 2410 | N | PHE | L | 94 | −13.933 | 41.014 | 23.390 | 1.00 | 18.65 | N |
| ATOM | 2411 | CA | PHE | L | 94 | −12.622 | 41.647 | 23.357 | 1.00 | 18.45 | C |
| ATOM | 2412 | CB | PHE | L | 94 | −12.698 | 43.089 | 22.843 | 1.00 | 18.19 | C |
| ATOM | 2413 | CG | PHE | L | 94 | −12.943 | 43.200 | 21.364 | 1.00 | 18.06 | C |
| ATOM | 2414 | CD1 | PHE | L | 94 | −11.890 | 43.110 | 20.462 | 1.00 | 17.06 | C |
| ATOM | 2415 | CE1 | PHE | L | 94 | −12.107 | 43.221 | 19.098 | 1.00 | 16.30 | C |
| ATOM | 2416 | CZ | PHE | L | 94 | −13.387 | 43.422 | 18.612 | 1.00 | 17.32 | C |
| ATOM | 2417 | CE2 | PHE | L | 94 | −14.458 | 43.521 | 19.499 | 1.00 | 17.93 | C |
| ATOM | 2418 | CD2 | PHE | L | 94 | −14.227 | 43.407 | 20.873 | 1.00 | 18.35 | C |
| ATOM | 2419 | C | PHE | L | 94 | −11.998 | 41.612 | 24.754 | 1.00 | 18.47 | C |
| ATOM | 2420 | O | PHE | L | 94 | −12.688 | 41.839 | 25.753 | 1.00 | 18.79 | O |
| ATOM | 2421 | N | PRO | L | 95 | −10.685 | 41.333 | 24.836 | 1.00 | 18.11 | N |
| ATOM | 2422 | CA | PRO | L | 95 | −9.773 | 41.037 | 23.730 | 1.00 | 17.62 | C |
| ATOM | 2423 | CB | PRO | L | 95 | −8.407 | 41.242 | 24.366 | 1.00 | 17.21 | C |
| ATOM | 2424 | CG | PRO | L | 95 | −8.614 | 40.853 | 25.779 | 1.00 | 17.83 | C |
| ATOM | 2425 | CD | PRO | L | 95 | −9.988 | 41.324 | 26.136 | 1.00 | 17.87 | C |
| ATOM | 2426 | C | PRO | L | 95 | −9.930 | 39.589 | 23.235 | 1.00 | 17.23 | C |
| ATOM | 2427 | O | PRO | L | 95 | −10.259 | 38.696 | 24.023 | 1.00 | 17.34 | O |
| ATOM | 2428 | N | PHE | L | 96 | −9.703 | 39.363 | 21.945 | 1.00 | 16.42 | N |
| ATOM | 2429 | CA | PHE | L | 96 | −9.803 | 38.019 | 21.408 | 1.00 | 15.67 | C |
| ATOM | 2430 | CB | PHE | L | 96 | −9.643 | 37.995 | 19.885 | 1.00 | 15.19 | C |
| ATOM | 2431 | CG | PHE | L | 96 | −10.752 | 38.710 | 19.119 | 1.00 | 13.89 | C |
| ATOM | 2432 | CD1 | PHE | L | 96 | −10.588 | 39.000 | 17.760 | 1.00 | 11.24 | C |
| ATOM | 2433 | CE1 | PHE | L | 96 | −11.600 | 39.662 | 17.034 | 1.00 | 11.07 | C |
| ATOM | 2434 | CZ | PHE | L | 96 | −12.796 | 40.039 | 17.674 | 1.00 | 10.74 | C |
| ATOM | 2435 | CE2 | PHE | L | 96 | −12.969 | 39.756 | 19.027 | 1.00 | 11.36 | C |
| ATOM | 2436 | CD2 | PHE | L | 96 | −11.948 | 39.100 | 19.747 | 1.00 | 12.18 | C |
| ATOM | 2437 | C | PHE | L | 96 | −8.750 | 37.147 | 22.070 | 1.00 | 15.90 | C |
| ATOM | 2438 | O | PHE | L | 96 | −7.642 | 37.583 | 22.365 | 1.00 | 15.94 | O |
| ATOM | 2439 | N | THR | L | 97 | −9.108 | 35.905 | 22.335 | 1.00 | 16.30 | N |
| ATOM | 2440 | CA | THR | L | 97 | −8.213 | 35.027 | 23.051 | 1.00 | 15.89 | C |
| ATOM | 2441 | CB | THR | L | 97 | −8.679 | 34.846 | 24.509 | 1.00 | 16.02 | C |
| ATOM | 2442 | OG1 | THR | L | 97 | −10.082 | 34.577 | 24.520 | 1.00 | 16.99 | O |
| ATOM | 2443 | CG2 | THR | L | 97 | −8.412 | 36.108 | 25.350 | 1.00 | 13.96 | C |
| ATOM | 2444 | C | THR | L | 97 | −8.101 | 33.676 | 22.357 | 1.00 | 16.14 | C |
| ATOM | 2445 | O | THR | L | 97 | −8.902 | 33.326 | 21.494 | 1.00 | 15.34 | O |
| ATOM | 2446 | N | PHE | L | 98 | −7.082 | 32.926 | 22.763 | 1.00 | 16.65 | N |
| ATOM | 2447 | CA | PHE | L | 98 | −6.788 | 31.625 | 22.212 | 1.00 | 16.65 | C |
| ATOM | 2448 | CB | PHE | L | 98 | −5.550 | 31.738 | 21.335 | 1.00 | 16.09 | C |
| ATOM | 2449 | CG | PHE | L | 98 | −5.780 | 32.481 | 20.048 | 1.00 | 16.06 | C |
| ATOM | 2450 | CD1 | PHE | L | 98 | −5.579 | 33.859 | 19.968 | 1.00 | 16.30 | C |
| ATOM | 2451 | CE1 | PHE | L | 98 | −5.781 | 34.540 | 18.778 | 1.00 | 13.72 | C |
| ATOM | 2452 | CZ | PHE | L | 98 | −6.175 | 33.853 | 17.663 | 1.00 | 13.78 | C |
| ATOM | 2453 | CE2 | PHE | L | 98 | −6.372 | 32.487 | 17.734 | 1.00 | 14.66 | C |
| ATOM | 2454 | CD2 | PHE | L | 98 | −6.174 | 31.809 | 18.914 | 1.00 | 13.45 | C |
| ATOM | 2455 | C | PHE | L | 98 | −6.509 | 30.637 | 23.331 | 1.00 | 17.23 | C |
| ATOM | 2456 | O | PHE | L | 98 | −5.981 | 31.013 | 24.378 | 1.00 | 17.55 | O |
| ATOM | 2457 | N | GLY | L | 99 | −6.848 | 29.372 | 23.120 | 1.00 | 17.75 | N |
| ATOM | 2458 | CA | GLY | L | 99 | −6.368 | 28.317 | 24.020 | 1.00 | 18.40 | C |
| ATOM | 2459 | C | GLY | L | 99 | −4.867 | 28.108 | 23.817 | 1.00 | 19.22 | C |
| ATOM | 2460 | O | GLY | L | 99 | −4.289 | 28.572 | 22.817 | 1.00 | 18.68 | O |
| ATOM | 2461 | N | GLN | L | 100 | −4.235 | 27.393 | 24.745 | 1.00 | 19.49 | N |
| ATOM | 2462 | CA | GLN | L | 100 | −2.791 | 27.223 | 24.695 | 1.00 | 20.32 | C |
| ATOM | 2463 | CB | GLN | L | 100 | −2.190 | 27.073 | 26.104 | 1.00 | 20.67 | C |
| ATOM | 2464 | CG | GLN | L | 100 | −2.265 | 28.396 | 26.962 | 1.00 | 23.75 | C |
| ATOM | 2465 | CD | GLN | L | 100 | −2.093 | 29.708 | 26.136 | 1.00 | 26.63 | C |
| ATOM | 2466 | OE1 | GLN | L | 100 | −1.067 | 29.913 | 25.468 | 1.00 | 25.89 | O |
| ATOM | 2467 | NE2 | GLN | L | 100 | −3.109 | 30.595 | 26.194 | 1.00 | 26.24 | N |
| ATOM | 2468 | C | GLN | L | 100 | −2.274 | 26.172 | 23.686 | 1.00 | 19.85 | C |
| ATOM | 2469 | O | GLN | L | 100 | −1.073 | 26.103 | 23.418 | 1.00 | 19.35 | O |
| ATOM | 2470 | N | GLY | L | 101 | −3.185 | 25.415 | 23.090 | 1.00 | 19.43 | N |
| ATOM | 2471 | CA | GLY | L | 101 | −2.816 | 24.465 | 22.056 | 1.00 | 19.76 | C |
| ATOM | 2472 | C | GLY | L | 101 | −2.837 | 23.021 | 22.520 | 1.00 | 20.54 | C |
| ATOM | 2473 | O | GLY | L | 101 | −2.524 | 22.720 | 23.684 | 1.00 | 20.35 | O |
| ATOM | 2474 | N | THR | L | 102 | −3.220 | 22.132 | 21.604 | 1.00 | 20.88 | N |
| ATOM | 2475 | CA | THR | L | 102 | −3.141 | 20.691 | 21.805 | 1.00 | 21.52 | C |
| ATOM | 2476 | CB | THR | L | 102 | −4.531 | 20.009 | 21.637 | 1.00 | 21.37 | C |
| ATOM | 2477 | OG1 | THR | L | 102 | −5.373 | 20.336 | 22.750 | 1.00 | 20.44 | O |
| ATOM | 2478 | CG2 | THR | L | 102 | −4.405 | 18.467 | 21.540 | 1.00 | 21.40 | C |
| ATOM | 2479 | C | THR | L | 102 | −2.195 | 20.145 | 20.750 | 1.00 | 22.29 | C |
| ATOM | 2480 | O | THR | L | 102 | −2.479 | 20.271 | 19.558 | 1.00 | 22.63 | O |
| ATOM | 2481 | N | LYS | L | 103 | −1.079 | 19.535 | 21.160 | 1.00 | 23.04 | N |
| ATOM | 2482 | CA | LYS | L | 103 | −0.179 | 18.924 | 20.159 | 1.00 | 23.73 | C |
| ATOM | 2483 | CB | LYS | L | 103 | 1.303 | 18.935 | 20.588 | 1.00 | 23.95 | C |
| ATOM | 2484 | CG | LYS | L | 103 | 2.283 | 18.804 | 19.380 | 1.00 | 26.46 | C |
| ATOM | 2485 | CD | LYS | L | 103 | 3.790 | 18.859 | 19.761 | 1.00 | 29.35 | C |
| ATOM | 2486 | CE | LYS | L | 103 | 4.338 | 20.306 | 19.763 | 1.00 | 30.82 | C |
| ATOM | 2487 | NZ | LYS | L | 103 | 5.794 | 20.387 | 19.416 | 1.00 | 31.59 | N |
| ATOM | 2488 | C | LYS | L | 103 | −0.640 | 17.528 | 19.720 | 1.00 | 23.24 | C |
| ATOM | 2489 | O | LYS | L | 103 | −0.726 | 16.603 | 20.527 | 1.00 | 23.44 | O |
| ATOM | 2490 | N | LEU | L | 104 | −0.956 | 17.393 | 18.438 | 1.00 | 22.87 | N |
| ATOM | 2491 | CA | LEU | L | 104 | −1.301 | 16.085 | 17.875 | 1.00 | 22.52 | C |
| ATOM | 2492 | CB | LEU | L | 104 | −2.524 | 16.193 | 16.965 | 1.00 | 22.29 | C |
| ATOM | 2493 | CG | LEU | L | 104 | −3.212 | 14.908 | 16.530 | 1.00 | 21.75 | C |
| ATOM | 2494 | CD1 | LEU | L | 104 | −4.701 | 15.104 | 16.257 | 1.00 | 19.99 | C |
| ATOM | 2495 | CD2 | LEU | L | 104 | −2.511 | 14.329 | 15.301 | 1.00 | 22.63 | C |
| ATOM | 2496 | C | LEU | L | 104 | −0.099 | 15.481 | 17.142 | 1.00 | 22.29 | C |
| ATOM | 2497 | O | LEU | L | 104 | 0.433 | 16.062 | 16.204 | 1.00 | 22.50 | O |
| ATOM | 2498 | N | GLU | L | 105 | 0.328 | 14.314 | 17.602 | 1.00 | 22.42 | N |
| ATOM | 2499 | CA | GLU | L | 105 | 1.489 | 13.627 | 17.058 | 1.00 | 22.34 | C |
| ATOM | 2500 | CB | GLU | L | 105 | 2.630 | 13.596 | 18.099 | 1.00 | 22.92 | C |
| ATOM | 2501 | CG | GLU | L | 105 | 2.352 | 12.696 | 19.332 | 1.00 | 25.31 | C |
| ATOM | 2502 | CD | GLU | L | 105 | 3.577 | 12.450 | 20.230 | 1.00 | 29.37 | C |
| ATOM | 2503 | OE1 | GLU | L | 105 | 4.097 | 11.311 | 20.226 | 1.00 | 32.06 | O |
| ATOM | 2504 | OE2 | GLU | L | 105 | 4.016 | 13.378 | 20.953 | 1.00 | 29.37 | O |
| ATOM | 2505 | C | GLU | L | 105 | 1.084 | 12.206 | 16.693 | 1.00 | 21.73 | C |
| ATOM | 2506 | O | GLU | L | 105 | 0.060 | 11.705 | 17.172 | 1.00 | 20.96 | O |
| ATOM | 2507 | N | ILE | L | 106 | 1.902 | 11.562 | 15.861 | 1.00 | 21.61 | N |
| ATOM | 2508 | CA | ILE | L | 106 | 1.689 | 10.173 | 15.449 | 1.00 | 21.16 | C |
| ATOM | 2509 | CB | ILE | L | 106 | 2.452 | 9.851 | 14.135 | 1.00 | 21.18 | C |
| ATOM | 2510 | CG1 | ILE | L | 106 | 1.847 | 10.622 | 12.955 | 1.00 | 19.44 | C |
| ATOM | 2511 | CD1 | ILE | L | 106 | 0.324 | 10.459 | 12.829 | 1.00 | 18.89 | C |
| ATOM | 2512 | CG2 | ILE | L | 106 | 2.465 | 8.335 | 13.857 | 1.00 | 19.42 | C |
| ATOM | 2513 | C | ILE | L | 106 | 2.125 | 9.215 | 16.546 | 1.00 | 21.53 | C |
| ATOM | 2514 | O | ILE | L | 106 | 3.219 | 9.346 | 17.076 | 1.00 | 21.54 | O |
| ATOM | 2515 | N | LYS | L | 107 | 1.258 | 8.270 | 16.899 | 1.00 | 21.78 | N |
| ATOM | 2516 | CA | LYS | L | 107 | 1.586 | 7.259 | 17.895 | 1.00 | 22.21 | C |
| ATOM | 2517 | CB | LYS | L | 107 | 0.342 | 6.895 | 18.720 | 1.00 | 22.78 | C |
| ATOM | 2518 | CG | LYS | L | 107 | 0.551 | 5.765 | 19.760 | 1.00 | 24.36 | C |
| ATOM | 2519 | CD | LYS | L | 107 | −0.633 | 5.659 | 20.703 | 1.00 | 28.21 | C |
| ATOM | 2520 | CE | LYS | L | 107 | −0.692 | 4.297 | 21.424 | 1.00 | 30.79 | C |
| ATOM | 2521 | NZ | LYS | L | 107 | 0.347 | 4.160 | 22.483 | 1.00 | 30.16 | N |
| ATOM | 2522 | C | LYS | L | 107 | 2.162 | 6.015 | 17.212 | 1.00 | 22.23 | C |
| ATOM | 2523 | O | LYS | L | 107 | 1.543 | 5.447 | 16.313 | 1.00 | 21.96 | O |
| ATOM | 2524 | N | ARG | L | 108 | 3.344 | 5.588 | 17.643 | 1.00 | 22.18 | N |
| ATOM | 2525 | CA | ARG | L | 108 | 3.974 | 4.408 | 17.055 | 1.00 | 22.35 | C |
| ATOM | 2526 | CB | ARG | L | 108 | 5.143 | 4.814 | 16.157 | 1.00 | 22.29 | C |
| ATOM | 2527 | CG | ARG | L | 108 | 6.217 | 5.599 | 16.865 | 1.00 | 22.52 | C |
| ATOM | 2528 | CD | ARG | L | 108 | 7.567 | 5.328 | 16.246 | 1.00 | 23.28 | C |
| ATOM | 2529 | NE | ARG | L | 108 | 7.999 | 3.943 | 16.452 | 1.00 | 24.35 | N |
| ATOM | 2530 | CZ | ARG | L | 108 | 8.945 | 3.335 | 15.740 | 1.00 | 23.98 | C |
| ATOM | 2531 | NH1 | ARG | L | 108 | 9.267 | 2.080 | 16.003 | 1.00 | 24.74 | N |
| ATOM | 2532 | NH2 | ARG | L | 108 | 9.564 | 3.975 | 14.761 | 1.00 | 23.61 | N |
| ATOM | 2533 | C | ARG | L | 108 | 4.427 | 3.456 | 18.152 | 1.00 | 22.50 | C |
| ATOM | 2534 | O | ARG | L | 108 | 4.217 | 3.729 | 19.335 | 1.00 | 22.88 | O |
| ATOM | 2535 | N | THR | L | 109 | 5.037 | 2.334 | 17.779 | 1.00 | 22.51 | N |
| ATOM | 2536 | CA | THR | L | 109 | 5.535 | 1.410 | 18.801 | 1.00 | 22.31 | C |
| ATOM | 2537 | CB | THR | L | 109 | 5.982 | 0.064 | 18.224 | 1.00 | 22.46 | C |
| ATOM | 2538 | OG1 | THR | L | 109 | 7.206 | 0.247 | 17.495 | 1.00 | 24.03 | O |
| ATOM | 2539 | CG2 | THR | L | 109 | 4.907 | −0.525 | 17.317 | 1.00 | 22.05 | C |
| ATOM | 2540 | C | THR | L | 109 | 6.727 | 2.045 | 19.515 | 1.00 | 21.75 | C |
| ATOM | 2541 | O | THR | L | 109 | 7.438 | 2.882 | 18.943 | 1.00 | 21.61 | O |
| ATOM | 2542 | N | VAL | L | 110 | 6.924 | 1.636 | 20.759 | 1.00 | 20.83 | N |
| ATOM | 2543 | CA | VAL | L | 110 | 8.001 | 2.113 | 21.576 | 1.00 | 20.68 | C |
| ATOM | 2544 | CB | VAL | L | 110 | 7.924 | 1.464 | 22.988 | 1.00 | 20.73 | C |
| ATOM | 2545 | CG1 | VAL | L | 110 | 9.222 | 1.664 | 23.763 | 1.00 | 20.73 | C |
| ATOM | 2546 | CG2 | VAL | L | 110 | 6.738 | 2.024 | 23.767 | 1.00 | 19.91 | C |
| ATOM | 2547 | C | VAL | L | 110 | 9.320 | 1.796 | 20.873 | 1.00 | 20.86 | C |
| ATOM | 2548 | O | VAL | L | 110 | 9.549 | 0.658 | 20.472 | 1.00 | 20.61 | O |
| ATOM | 2549 | N | ALA | L | 111 | 10.160 | 2.817 | 20.694 | 1.00 | 21.05 | N |
| ATOM | 2550 | CA | ALA | L | 111 | 11.489 | 2.652 | 20.093 | 1.00 | 21.05 | C |
| ATOM | 2551 | CB | ALA | L | 111 | 11.551 | 3.264 | 18.719 | 1.00 | 20.73 | C |
| ATOM | 2552 | C | ALA | L | 111 | 12.527 | 3.280 | 20.987 | 1.00 | 21.39 | C |
| ATOM | 2553 | O | ALA | L | 111 | 12.410 | 4.450 | 21.362 | 1.00 | 21.42 | O |
| ATOM | 2554 | N | ALA | L | 112 | 13.535 | 2.483 | 21.338 | 1.00 | 21.73 | N |
| ATOM | 2555 | CA | ALA | L | 112 | 14.602 | 2.918 | 22.229 | 1.00 | 21.70 | C |
| ATOM | 2556 | CB | ALA | L | 112 | 15.335 | 1.708 | 22.808 | 1.00 | 22.02 | C |
| ATOM | 2557 | C | ALA | L | 112 | 15.577 | 3.811 | 21.481 | 1.00 | 21.51 | C |
| ATOM | 2558 | O | ALA | L | 112 | 15.832 | 3.594 | 20.298 | 1.00 | 21.54 | O |
| ATOM | 2559 | N | PRO | L | 113 | 16.137 | 4.810 | 22.172 | 1.00 | 21.11 | N |
| ATOM | 2560 | CA | PRO | L | 113 | 17.156 | 5.620 | 21.539 | 1.00 | 21.08 | C |
| ATOM | 2561 | CB | PRO | L | 113 | 17.400 | 6.729 | 22.558 | 1.00 | 20.80 | C |
| ATOM | 2562 | CG | PRO | L | 113 | 17.018 | 6.153 | 23.858 | 1.00 | 20.88 | C |
| ATOM | 2563 | CD | PRO | L | 113 | 15.897 | 5.198 | 23.573 | 1.00 | 20.99 | C |
| ATOM | 2564 | C | PRO | L | 113 | 18.455 | 4.848 | 21.315 | 1.00 | 21.76 | C |
| ATOM | 2565 | O | PRO | L | 113 | 18.776 | 3.924 | 22.081 | 1.00 | 22.27 | O |
| ATOM | 2566 | N | SER | L | 114 | 19.179 | 5.207 | 20.258 | 1.00 | 21.67 | N |
| ATOM | 2567 | CA | SER | L | 114 | 20.590 | 4.886 | 20.171 | 1.00 | 21.79 | C |
| ATOM | 2568 | CB | SER | L | 114 | 21.047 | 4.721 | 18.719 | 1.00 | 22.03 | C |
| ATOM | 2569 | OG | SER | L | 114 | 20.543 | 3.518 | 18.161 | 1.00 | 22.85 | O |
| ATOM | 2570 | C | SER | L | 114 | 21.287 | 6.073 | 20.802 | 1.00 | 21.81 | C |
| ATOM | 2571 | O | SER | L | 114 | 20.999 | 7.236 | 20.460 | 1.00 | 22.22 | O |
| ATOM | 2572 | N | VAL | L | 115 | 22.187 | 5.793 | 21.737 | 1.00 | 21.25 | N |
| ATOM | 2573 | CA | VAL | L | 115 | 22.853 | 6.852 | 22.461 | 1.00 | 20.39 | C |
| ATOM | 2574 | CB | VAL | L | 115 | 22.783 | 6.623 | 23.979 | 1.00 | 20.31 | C |
| ATOM | 2575 | CG1 | VAL | L | 115 | 23.323 | 7.828 | 24.729 | 1.00 | 19.96 | C |
| ATOM | 2576 | CG2 | VAL | L | 115 | 21.357 | 6.334 | 24.402 | 1.00 | 18.88 | C |
| ATOM | 2577 | C | VAL | L | 115 | 24.281 | 6.908 | 21.994 | 1.00 | 20.73 | C |
| ATOM | 2578 | O | VAL | L | 115 | 24.943 | 5.872 | 21.908 | 1.00 | 20.99 | O |
| ATOM | 2579 | N | PHE | L | 116 | 24.751 | 8.116 | 21.679 | 1.00 | 20.82 | N |
| ATOM | 2580 | CA | PHE | L | 116 | 26.151 | 8.348 | 21.320 | 1.00 | 20.75 | C |
| ATOM | 2581 | CB | PHE | L | 116 | 26.317 | 8.638 | 19.822 | 1.00 | 20.76 | C |
| ATOM | 2582 | CG | PHE | L | 116 | 25.666 | 7.627 | 18.905 | 1.00 | 20.87 | C |
| ATOM | 2583 | CD1 | PHE | L | 116 | 26.420 | 6.589 | 18.345 | 1.00 | 22.28 | C |
| ATOM | 2584 | CE1 | PHE | L | 116 | 25.825 | 5.654 | 17.475 | 1.00 | 22.87 | C |
| ATOM | 2585 | CZ | PHE | L | 116 | 24.456 | 5.778 | 17.159 | 1.00 | 22.29 | C |
| ATOM | 2586 | CE2 | PHE | L | 116 | 23.710 | 6.841 | 17.708 | 1.00 | 19.85 | C |
| ATOM | 2587 | CD2 | PHE | L | 116 | 24.315 | 7.746 | 18.559 | 1.00 | 18.77 | C |
| ATOM | 2588 | C | PHE | L | 116 | 26.658 | 9.555 | 22.096 | 1.00 | 21.12 | C |
| ATOM | 2589 | O | PHE | L | 116 | 25.900 | 10.509 | 22.348 | 1.00 | 21.00 | O |
| ATOM | 2590 | N | ILE | L | 117 | 27.942 | 9.534 | 22.449 | 1.00 | 21.17 | N |
| ATOM | 2591 | CA | ILE | L | 117 | 28.539 | 10.637 | 23.193 | 1.00 | 21.69 | C |
| ATOM | 2592 | CB | ILE | L | 117 | 28.820 | 10.260 | 24.675 | 1.00 | 21.85 | C |
| ATOM | 2593 | CG1 | ILE | L | 117 | 29.077 | 11.528 | 25.505 | 1.00 | 21.64 | C |
| ATOM | 2594 | CD1 | ILE | L | 117 | 29.536 | 11.263 | 26.943 | 1.00 | 21.25 | C |
| ATOM | 2595 | CG2 | ILE | L | 117 | 29.941 | 9.159 | 24.789 | 1.00 | 21.68 | C |
| ATOM | 2596 | C | ILE | L | 117 | 29.795 | 11.149 | 22.515 | 1.00 | 22.05 | C |
| ATOM | 2597 | O | ILE | L | 117 | 30.629 | 10.366 | 22.072 | 1.00 | 22.81 | O |
| ATOM | 2598 | N | PHE | L | 118 | 29.915 | 12.469 | 22.426 | 1.00 | 22.23 | N |
| ATOM | 2599 | CA | PHE | L | 118 | 31.062 | 13.093 | 21.789 | 1.00 | 22.29 | C |
| ATOM | 2600 | CB | PHE | L | 118 | 30.631 | 13.898 | 20.575 | 1.00 | 21.80 | C |
| ATOM | 2601 | CG | PHE | L | 118 | 29.770 | 13.136 | 19.604 | 1.00 | 21.29 | C |
| ATOM | 2602 | CD1 | PHE | L | 118 | 30.348 | 12.412 | 18.556 | 1.00 | 20.59 | C |
| ATOM | 2603 | CE1 | PHE | L | 118 | 29.547 | 11.709 | 17.645 | 1.00 | 19.17 | C |
| ATOM | 2604 | CZ | PHE | L | 118 | 28.153 | 11.751 | 17.772 | 1.00 | 18.62 | C |
| ATOM | 2605 | CE2 | PHE | L | 118 | 27.569 | 12.476 | 18.805 | 1.00 | 18.28 | C |
| ATOM | 2606 | CD2 | PHE | L | 118 | 28.375 | 13.163 | 19.714 | 1.00 | 19.78 | C |
| ATOM | 2607 | C | PHE | L | 118 | 31.821 | 13.998 | 22.760 | 1.00 | 23.13 | C |
| ATOM | 2608 | O | PHE | L | 118 | 31.276 | 14.988 | 23.257 | 1.00 | 22.83 | O |
| ATOM | 2609 | N | PRO | L | 119 | 33.082 | 13.647 | 23.046 | 1.00 | 23.72 | N |
| ATOM | 2610 | CA | PRO | L | 119 | 33.993 | 14.507 | 23.783 | 1.00 | 24.77 | C |
| ATOM | 2611 | CB | PRO | L | 119 | 35.315 | 13.716 | 23.788 | 1.00 | 24.65 | C |
| ATOM | 2612 | CG | PRO | L | 119 | 35.114 | 12.566 | 22.873 | 1.00 | 23.91 | C |
| ATOM | 2613 | CD | PRO | L | 119 | 33.651 | 12.315 | 22.808 | 1.00 | 23.69 | C |
| ATOM | 2614 | C | PRO | L | 119 | 34.198 | 15.804 | 23.036 | 1.00 | 25.74 | C |
| ATOM | 2615 | O | PRO | L | 119 | 33.943 | 15.847 | 21.848 | 1.00 | 26.23 | O |
| ATOM | 2616 | N | PRO | L | 120 | 34.668 | 16.856 | 23.719 | 1.00 | 26.97 | N |
| ATOM | 2617 | CA | PRO | L | 120 | 34.983 | 18.080 | 22.981 | 1.00 | 27.88 | C |
| ATOM | 2618 | CB | PRO | L | 120 | 35.319 | 19.092 | 24.080 | 1.00 | 27.73 | C |
| ATOM | 2619 | CG | PRO | L | 120 | 35.663 | 18.262 | 25.280 | 1.00 | 27.82 | C |
| ATOM | 2620 | CD | PRO | L | 120 | 34.842 | 17.013 | 25.175 | 1.00 | 26.85 | C |
| ATOM | 2621 | C | PRO | L | 120 | 36.182 | 17.908 | 22.060 | 1.00 | 29.27 | C |
| ATOM | 2622 | O | PRO | L | 120 | 37.092 | 17.106 | 22.338 | 1.00 | 29.67 | O |
| ATOM | 2623 | N | SER | L | 121 | 36.176 | 18.673 | 20.974 | 1.00 | 30.26 | N |
| ATOM | 2624 | CA | SER | L | 121 | 37.263 | 18.681 | 20.008 | 1.00 | 30.96 | C |
| ATOM | 2625 | CB | SER | L | 121 | 36.774 | 19.348 | 18.724 | 1.00 | 30.92 | C |
| ATOM | 2626 | OG | SER | L | 121 | 36.623 | 20.746 | 18.925 | 1.00 | 29.99 | O |
| ATOM | 2627 | C | SER | L | 121 | 38.470 | 19.452 | 20.526 | 1.00 | 31.82 | C |
| ATOM | 2628 | O | SER | L | 121 | 38.330 | 20.379 | 21.328 | 1.00 | 31.30 | O |
| ATOM | 2629 | N | ASP | L | 122 | 39.650 | 19.084 | 20.024 | 1.00 | 33.33 | N |
| ATOM | 2630 | CA | ASP | L | 122 | 40.889 | 19.829 | 20.275 | 1.00 | 34.62 | C |
| ATOM | 2631 | CB | ASP | L | 122 | 42.061 | 19.230 | 19.481 | 1.00 | 35.34 | C |
| ATOM | 2632 | CG | ASP | L | 122 | 42.551 | 17.896 | 20.048 | 1.00 | 37.32 | C |
| ATOM | 2633 | OD1 | ASP | L | 122 | 42.656 | 17.755 | 21.295 | 1.00 | 39.03 | O |
| ATOM | 2634 | OD2 | ASP | L | 122 | 42.851 | 16.993 | 19.234 | 1.00 | 38.85 | O |
| ATOM | 2635 | C | ASP | L | 122 | 40.719 | 21.286 | 19.885 | 1.00 | 34.90 | C |
| ATOM | 2636 | O | ASP | L | 122 | 41.168 | 22.185 | 20.602 | 1.00 | 35.13 | O |
| ATOM | 2637 | N | GLU | L | 123 | 40.067 | 21.507 | 18.746 | 1.00 | 35.16 | N |
| ATOM | 2638 | CA | GLU | L | 123 | 39.789 | 22.851 | 18.240 | 1.00 | 35.76 | C |
| ATOM | 2639 | CB | GLU | L | 123 | 39.022 | 22.793 | 16.905 | 1.00 | 36.27 | C |
| ATOM | 2640 | CG | GLU | L | 123 | 39.726 | 22.062 | 15.752 | 1.00 | 38.04 | C |
| ATOM | 2641 | CD | GLU | L | 123 | 39.416 | 20.567 | 15.690 | 1.00 | 41.00 | C |
| ATOM | 2642 | OE1 | GLU | L | 123 | 39.537 | 19.869 | 16.734 | 1.00 | 41.04 | O |
| ATOM | 2643 | OE2 | GLU | L | 123 | 39.078 | 20.089 | 14.577 | 1.00 | 41.80 | O |
| ATOM | 2644 | C | GLU | L | 123 | 39.006 | 23.694 | 19.247 | 1.00 | 35.49 | C |
| ATOM | 2645 | O | GLU | L | 123 | 39.312 | 24.872 | 19.440 | 1.00 | 35.69 | O |
| ATOM | 2646 | N | GLN | L | 124 | 38.001 | 23.098 | 19.885 | 1.00 | 35.44 | N |
| ATOM | 2647 | CA | GLN | L | 124 | 37.200 | 23.832 | 20.860 | 1.00 | 35.45 | C |
| ATOM | 2648 | CB | GLN | L | 124 | 35.913 | 23.086 | 21.225 | 1.00 | 35.11 | C |
| ATOM | 2649 | CG | GLN | L | 124 | 34.952 | 23.945 | 22.051 | 1.00 | 34.11 | C |
| ATOM | 2650 | CD | GLN | L | 124 | 33.716 | 23.205 | 22.534 | 1.00 | 32.57 | C |
| ATOM | 2651 | OE1 | GLN | L | 124 | 33.603 | 21.986 | 22.395 | 1.00 | 31.45 | O |
| ATOM | 2652 | NE2 | GLN | L | 124 | 32.778 | 23.949 | 23.093 | 1.00 | 29.97 | N |
| ATOM | 2653 | C | GLN | L | 124 | 37.999 | 24.145 | 22.113 | 1.00 | 35.77 | C |
| ATOM | 2654 | O | GLN | L | 124 | 37.889 | 25.239 | 22.668 | 1.00 | 35.70 | O |
| ATOM | 2655 | N | LEU | L | 125 | 38.797 | 23.181 | 22.559 | 1.00 | 36.52 | N |
| ATOM | 2656 | CA | LEU | L | 125 | 39.613 | 23.361 | 23.757 | 1.00 | 37.25 | C |
| ATOM | 2657 | CB | LEU | L | 125 | 40.345 | 22.069 | 24.141 | 1.00 | 37.27 | C |
| ATOM | 2658 | CG | LEU | L | 125 | 39.661 | 20.986 | 24.996 | 1.00 | 37.03 | C |
| ATOM | 2659 | CD1 | LEU | L | 125 | 39.229 | 19.802 | 24.160 | 1.00 | 36.84 | C |
| ATOM | 2660 | CD2 | LEU | L | 125 | 38.502 | 21.517 | 25.856 | 1.00 | 36.15 | C |
| ATOM | 2661 | C | LEU | L | 125 | 40.600 | 24.522 | 23.638 | 1.00 | 38.06 | C |
| ATOM | 2662 | O | LEU | L | 125 | 40.785 | 25.259 | 24.612 | 1.00 | 38.24 | O |
| ATOM | 2663 | N | LYS | L | 126 | 41.209 | 24.695 | 22.454 | 1.00 | 38.74 | N |
| ATOM | 2664 | CA | LYS | L | 126 | 42.066 | 25.862 | 22.175 | 1.00 | 39.52 | C |
| ATOM | 2665 | CB | LYS | L | 126 | 42.635 | 25.849 | 20.746 | 1.00 | 39.88 | C |
| ATOM | 2666 | CG | LYS | L | 126 | 43.666 | 24.751 | 20.448 | 1.00 | 41.78 | C |
| ATOM | 2667 | CD | LYS | L | 126 | 44.614 | 25.120 | 19.291 | 1.00 | 44.51 | C |
| ATOM | 2668 | CE | LYS | L | 126 | 43.956 | 25.065 | 17.903 | 1.00 | 46.75 | C |
| ATOM | 2669 | NZ | LYS | L | 126 | 43.711 | 23.673 | 17.383 | 1.00 | 48.19 | N |
| ATOM | 2670 | C | LYS | L | 126 | 41.323 | 27.178 | 22.395 | 1.00 | 39.57 | C |
| ATOM | 2671 | O | LYS | L | 126 | 41.926 | 28.173 | 22.800 | 1.00 | 39.86 | O |
| ATOM | 2672 | N | SER | L | 127 | 40.015 | 27.178 | 22.131 | 1.00 | 39.49 | N |
| ATOM | 2673 | CA | SER | L | 127 | 39.196 | 28.384 | 22.267 | 1.00 | 39.02 | C |
| ATOM | 2674 | CB | SER | L | 127 | 37.851 | 28.222 | 21.536 | 1.00 | 39.47 | C |
| ATOM | 2675 | OG | SER | L | 127 | 36.841 | 27.682 | 22.390 | 1.00 | 40.55 | O |
| ATOM | 2676 | C | SER | L | 127 | 38.968 | 28.765 | 23.732 | 1.00 | 38.16 | C |
| ATOM | 2677 | O | SER | L | 127 | 38.624 | 29.910 | 24.032 | 1.00 | 38.57 | O |
| ATOM | 2678 | N | GLY | L | 128 | 39.145 | 27.810 | 24.638 | 1.00 | 36.85 | N |
| ATOM | 2679 | CA | GLY | L | 128 | 38.961 | 28.079 | 26.064 | 1.00 | 35.06 | C |
| ATOM | 2680 | C | GLY | L | 128 | 37.744 | 27.420 | 26.681 | 1.00 | 33.92 | C |
| ATOM | 2681 | O | GLY | L | 128 | 37.511 | 27.554 | 27.886 | 1.00 | 34.02 | O |
| ATOM | 2682 | N | THR | L | 129 | 36.978 | 26.690 | 25.864 | 1.00 | 32.50 | N |
| ATOM | 2683 | CA | THR | L | 129 | 35.717 | 26.093 | 26.307 | 1.00 | 30.96 | C |
| ATOM | 2684 | CB | THR | L | 129 | 34.500 | 26.855 | 25.715 | 1.00 | 31.14 | C |
| ATOM | 2685 | OG1 | THR | L | 129 | 34.659 | 28.262 | 25.937 | 1.00 | 31.28 | O |
| ATOM | 2686 | CG2 | THR | L | 129 | 33.186 | 26.405 | 26.351 | 1.00 | 31.03 | C |
| ATOM | 2687 | C | THR | L | 129 | 35.636 | 24.611 | 25.955 | 1.00 | 30.00 | C |
| ATOM | 2688 | O | THR | L | 129 | 36.304 | 24.152 | 25.031 | 1.00 | 29.50 | O |
| ATOM | 2689 | N | ALA | L | 130 | 34.830 | 23.869 | 26.719 | 1.00 | 28.72 | N |
| ATOM | 2690 | CA | ALA | L | 130 | 34.553 | 22.464 | 26.435 | 1.00 | 27.63 | C |
| ATOM | 2691 | CB | ALA | L | 130 | 35.228 | 21.565 | 27.460 | 1.00 | 27.29 | C |
| ATOM | 2692 | C | ALA | L | 130 | 33.051 | 22.193 | 26.401 | 1.00 | 26.80 | C |
| ATOM | 2693 | O | ALA | L | 130 | 32.345 | 22.445 | 27.378 | 1.00 | 26.61 | O |
| ATOM | 2694 | N | SER | L | 131 | 32.581 | 21.680 | 25.267 | 1.00 | 25.69 | N |
| ATOM | 2695 | CA | SER | L | 131 | 31.192 | 21.281 | 25.102 | 1.00 | 24.31 | C |
| ATOM | 2696 | CB | SER | L | 131 | 30.549 | 21.961 | 23.895 | 1.00 | 24.28 | C |
| ATOM | 2697 | OG | SER | L | 131 | 30.382 | 23.351 | 24.095 | 1.00 | 23.43 | O |
| ATOM | 2698 | C | SER | L | 131 | 31.185 | 19.800 | 24.876 | 1.00 | 23.70 | C |
| ATOM | 2699 | O | SER | L | 131 | 31.805 | 19.320 | 23.931 | 1.00 | 23.56 | O |
| ATOM | 2700 | N | VAL | L | 132 | 30.489 | 19.086 | 25.756 | 1.00 | 22.71 | N |
| ATOM | 2701 | CA | VAL | L | 132 | 30.313 | 17.658 | 25.641 | 1.00 | 21.37 | C |
| ATOM | 2702 | CB | VAL | L | 132 | 30.510 | 16.953 | 26.996 | 1.00 | 21.41 | C |
| ATOM | 2703 | CG1 | VAL | L | 132 | 30.756 | 15.472 | 26.791 | 1.00 | 20.86 | C |
| ATOM | 2704 | CG2 | VAL | L | 132 | 31.636 | 17.571 | 27.791 | 1.00 | 20.28 | C |
| ATOM | 2705 | C | VAL | L | 132 | 28.873 | 17.437 | 25.186 | 1.00 | 21.34 | C |
| ATOM | 2706 | O | VAL | L | 132 | 27.946 | 18.037 | 25.722 | 1.00 | 20.79 | O |
| ATOM | 2707 | N | VAL | L | 133 | 28.692 | 16.564 | 24.201 | 1.00 | 21.26 | N |
| ATOM | 2708 | CA | VAL | L | 133 | 27.406 | 16.388 | 23.543 | 1.00 | 21.01 | C |
| ATOM | 2709 | CB | VAL | L | 133 | 27.509 | 16.801 | 22.057 | 1.00 | 20.71 | C |
| ATOM | 2710 | CG1 | VAL | L | 133 | 26.242 | 16.441 | 21.285 | 1.00 | 20.08 | C |
| ATOM | 2711 | CG2 | VAL | L | 133 | 27.805 | 18.288 | 21.958 | 1.00 | 20.46 | C |
| ATOM | 2712 | C | VAL | L | 133 | 26.934 | 14.950 | 23.643 | 1.00 | 21.33 | C |
| ATOM | 2713 | O | VAL | L | 133 | 27.673 | 14.033 | 23.268 | 1.00 | 21.16 | O |
| ATOM | 2714 | N | CYS | L | 134 | 25.713 | 14.765 | 24.152 | 1.00 | 21.59 | N |
| ATOM | 2715 | CA | CYS | L | 134 | 25.070 | 13.454 | 24.176 | 1.00 | 22.46 | C |
| ATOM | 2716 | CB | CYS | L | 134 | 24.604 | 13.092 | 25.577 | 1.00 | 22.68 | C |
| ATOM | 2717 | SG | CYS | L | 134 | 23.953 | 11.390 | 25.676 | 1.00 | 24.33 | S |
| ATOM | 2718 | C | CYS | L | 134 | 23.875 | 13.397 | 23.229 | 1.00 | 22.65 | C |
| ATOM | 2719 | O | CYS | L | 134 | 22.977 | 14.249 | 23.290 | 1.00 | 22.87 | O |
| ATOM | 2720 | N | LEU | L | 135 | 23.858 | 12.380 | 22.375 | 1.00 | 22.33 | N |
| ATOM | 2721 | CA | LEU | L | 135 | 22.825 | 12.252 | 21.358 | 1.00 | 22.22 | C |
| ATOM | 2722 | CB | LEU | L | 135 | 23.435 | 12.254 | 19.945 | 1.00 | 21.97 | C |
| ATOM | 2723 | CG | LEU | L | 135 | 22.620 | 11.660 | 18.784 | 1.00 | 22.24 | C |
| ATOM | 2724 | CD1 | LEU | L | 135 | 21.437 | 12.531 | 18.407 | 1.00 | 20.94 | C |
| ATOM | 2725 | CD2 | LEU | L | 135 | 23.493 | 11.420 | 17.552 | 1.00 | 22.39 | C |
| ATOM | 2726 | C | LEU | L | 135 | 21.996 | 11.003 | 21.589 | 1.00 | 22.20 | C |
| ATOM | 2727 | O | LEU | L | 135 | 22.545 | 9.914 | 21.717 | 1.00 | 22.55 | O |
| ATOM | 2728 | N | LEU | L | 136 | 20.677 | 11.197 | 21.653 | 1.00 | 22.13 | N |
| ATOM | 2729 | CA | LEU | L | 136 | 19.679 | 10.135 | 21.745 | 1.00 | 22.17 | C |
| ATOM | 2730 | CB | LEU | L | 136 | 18.685 | 10.403 | 22.881 | 1.00 | 21.88 | C |
| ATOM | 2731 | CG | LEU | L | 136 | 19.132 | 10.299 | 24.342 | 1.00 | 22.17 | C |
| ATOM | 2732 | CD1 | LEU | L | 136 | 20.173 | 11.349 | 24.723 | 1.00 | 21.20 | C |
| ATOM | 2733 | CD2 | LEU | L | 136 | 17.923 | 10.428 | 25.231 | 1.00 | 21.05 | C |
| ATOM | 2734 | C | LEU | L | 136 | 18.927 | 10.111 | 20.419 | 1.00 | 22.44 | C |
| ATOM | 2735 | O | LEU | L | 136 | 18.232 | 11.079 | 20.060 | 1.00 | 22.14 | O |
| ATOM | 2736 | N | ASN | L | 137 | 19.057 | 9.006 | 19.698 | 1.00 | 22.46 | N |
| ATOM | 2737 | CA | ASN | L | 137 | 18.660 | 8.998 | 18.304 | 1.00 | 22.98 | C |
| ATOM | 2738 | CB | ASN | L | 137 | 19.840 | 8.598 | 17.406 | 1.00 | 23.26 | C |
| ATOM | 2739 | CG | ASN | L | 137 | 19.674 | 9.097 | 15.977 | 1.00 | 25.27 | C |
| ATOM | 2740 | OD1 | ASN | L | 137 | 19.503 | 10.302 | 15.743 | 1.00 | 27.43 | O |
| ATOM | 2741 | ND2 | ASN | L | 137 | 19.714 | 8.176 | 15.016 | 1.00 | 24.57 | N |
| ATOM | 2742 | C | ASN | L | 137 | 17.433 | 8.164 | 17.972 | 1.00 | 22.49 | C |
| ATOM | 2743 | O | ASN | L | 137 | 17.287 | 7.029 | 18.442 | 1.00 | 22.23 | O |
| ATOM | 2744 | N | ASN | L | 138 | 16.567 | 8.739 | 17.139 | 1.00 | 21.93 | N |
| ATOM | 2745 | CA | ASN | L | 138 | 15.387 | 8.043 | 16.627 | 1.00 | 21.86 | C |
| ATOM | 2746 | CB | ASN | L | 138 | 15.732 | 7.212 | 15.369 | 1.00 | 21.81 | C |
| ATOM | 2747 | CG | ASN | L | 138 | 16.528 | 8.015 | 14.292 | 1.00 | 23.41 | C |
| ATOM | 2748 | OD1 | ASN | L | 138 | 16.733 | 9.236 | 14.394 | 1.00 | 23.09 | O |
| ATOM | 2749 | ND2 | ASN | L | 138 | 16.993 | 7.301 | 13.266 | 1.00 | 23.13 | N |
| ATOM | 2750 | C | ASN | L | 138 | 14.673 | 7.175 | 17.691 | 1.00 | 21.37 | C |
| ATOM | 2751 | O | ASN | L | 138 | 14.587 | 5.955 | 17.553 | 1.00 | 21.60 | O |
| ATOM | 2752 | N | PHE | L | 139 | 14.162 | 7.821 | 18.738 | 1.00 | 20.64 | N |
| ATOM | 2753 | CA | PHE | L | 139 | 13.386 | 7.150 | 19.776 | 1.00 | 20.26 | C |
| ATOM | 2754 | CB | PHE | L | 139 | 14.020 | 7.352 | 21.158 | 1.00 | 19.95 | C |
| ATOM | 2755 | CG | PHE | L | 139 | 14.084 | 8.783 | 21.605 | 1.00 | 17.92 | C |
| ATOM | 2756 | CD1 | PHE | L | 139 | 15.202 | 9.557 | 21.331 | 1.00 | 16.82 | C |
| ATOM | 2757 | CE1 | PHE | L | 139 | 15.275 | 10.878 | 21.741 | 1.00 | 16.86 | C |
| ATOM | 2758 | CZ | PHE | L | 139 | 14.220 | 11.434 | 22.458 | 1.00 | 17.10 | C |
| ATOM | 2759 | CE2 | PHE | L | 139 | 13.095 | 10.658 | 22.751 | 1.00 | 16.62 | C |
| ATOM | 2760 | CD2 | PHE | L | 139 | 13.036 | 9.345 | 22.319 | 1.00 | 16.63 | C |
| ATOM | 2761 | C | PHE | L | 139 | 11.913 | 7.577 | 19.814 | 1.00 | 20.58 | C |
| ATOM | 2762 | O | PHE | L | 139 | 11.522 | 8.566 | 19.204 | 1.00 | 20.76 | O |
| ATOM | 2763 | N | TYR | L | 140 | 11.111 | 6.811 | 20.539 | 1.00 | 20.80 | N |
| ATOM | 2764 | CA | TYR | L | 140 | 9.708 | 7.103 | 20.750 | 1.00 | 21.19 | C |
| ATOM | 2765 | CB | TYR | L | 140 | 8.840 | 6.646 | 19.562 | 1.00 | 20.70 | C |
| ATOM | 2766 | CG | TYR | L | 140 | 7.415 | 7.109 | 19.739 | 1.00 | 19.96 | C |
| ATOM | 2767 | CD1 | TYR | L | 140 | 6.471 | 6.283 | 20.340 | 1.00 | 18.15 | C |
| ATOM | 2768 | CE1 | TYR | L | 140 | 5.185 | 6.720 | 20.569 | 1.00 | 17.18 | C |
| ATOM | 2769 | CZ | TYR | L | 140 | 4.812 | 7.995 | 20.199 | 1.00 | 18.15 | C |
| ATOM | 2770 | OH | TYR | L | 140 | 3.509 | 8.384 | 20.427 | 1.00 | 19.12 | O |
| ATOM | 2771 | CE2 | TYR | L | 140 | 5.726 | 8.856 | 19.595 | 1.00 | 17.83 | C |
| ATOM | 2772 | CD2 | TYR | L | 140 | 7.028 | 8.415 | 19.382 | 1.00 | 18.84 | C |
| ATOM | 2773 | C | TYR | L | 140 | 9.261 | 6.388 | 22.017 | 1.00 | 22.03 | C |
| ATOM | 2774 | O | TYR | L | 140 | 9.665 | 5.253 | 22.245 | 1.00 | 22.70 | O |
| ATOM | 2775 | N | PRO | L | 141 | 8.419 | 7.030 | 22.844 | 1.00 | 22.95 | N |
| ATOM | 2776 | CA | PRO | L | 141 | 7.867 | 8.391 | 22.749 | 1.00 | 23.67 | C |
| ATOM | 2777 | CB | PRO | L | 141 | 6.618 | 8.316 | 23.640 | 1.00 | 23.87 | C |
| ATOM | 2778 | CG | PRO | L | 141 | 6.940 | 7.267 | 24.663 | 1.00 | 23.20 | C |
| ATOM | 2779 | CD | PRO | L | 141 | 7.776 | 6.250 | 23.920 | 1.00 | 23.07 | C |
| ATOM | 2780 | C | PRO | L | 141 | 8.846 | 9.477 | 23.224 | 1.00 | 24.63 | C |
| ATOM | 2781 | O | PRO | L | 141 | 10.008 | 9.171 | 23.489 | 1.00 | 24.14 | O |
| ATOM | 2782 | N | ARG | L | 142 | 8.379 | 10.725 | 23.324 | 1.00 | 26.13 | N |
| ATOM | 2783 | CA | ARG | L | 142 | 9.267 | 11.854 | 23.604 | 1.00 | 28.07 | C |
| ATOM | 2784 | CB | ARG | L | 142 | 8.605 | 13.220 | 23.314 | 1.00 | 28.76 | C |
| ATOM | 2785 | CG | ARG | L | 142 | 9.626 | 14.386 | 23.406 | 1.00 | 32.46 | C |
| ATOM | 2786 | CD | ARG | L | 142 | 9.043 | 15.780 | 23.203 | 1.00 | 37.95 | C |
| ATOM | 2787 | NE | ARG | L | 142 | 9.059 | 16.166 | 21.791 | 1.00 | 41.85 | N |
| ATOM | 2788 | CZ | ARG | L | 142 | 9.118 | 17.422 | 21.343 | 1.00 | 44.39 | C |
| ATOM | 2789 | NH1 | ARG | L | 142 | 9.185 | 18.444 | 22.204 | 1.00 | 44.71 | N |
| ATOM | 2790 | NH2 | ARG | L | 142 | 9.136 | 17.656 | 20.023 | 1.00 | 44.97 | N |
| ATOM | 2791 | C | ARG | L | 142 | 9.854 | 11.833 | 25.014 | 1.00 | 28.31 | C |
| ATOM | 2792 | O | ARG | L | 142 | 11.012 | 12.192 | 25.198 | 1.00 | 28.71 | O |
| ATOM | 2793 | N | GLU | L | 143 | 9.066 | 11.410 | 25.996 | 1.00 | 28.87 | N |
| ATOM | 2794 | CA | GLU | L | 143 | 9.528 | 11.314 | 27.378 | 1.00 | 29.73 | C |
| ATOM | 2795 | CB | GLU | L | 143 | 8.472 | 10.607 | 28.235 | 1.00 | 30.36 | C |
| ATOM | 2796 | CG | GLU | L | 143 | 8.727 | 10.634 | 29.752 | 1.00 | 34.75 | C |
| ATOM | 2797 | CD | GLU | L | 143 | 7.587 | 9.996 | 30.559 | 1.00 | 40.93 | C |
| ATOM | 2798 | OE1 | GLU | L | 143 | 6.937 | 9.042 | 30.046 | 1.00 | 43.22 | O |
| ATOM | 2799 | OE2 | GLU | L | 143 | 7.341 | 10.450 | 31.707 | 1.00 | 42.63 | O |
| ATOM | 2800 | C | GLU | L | 143 | 10.875 | 10.587 | 27.478 | 1.00 | 29.36 | C |
| ATOM | 2801 | O | GLU | L | 143 | 10.961 | 9.386 | 27.262 | 1.00 | 29.32 | O |
| ATOM | 2802 | N | ALA | L | 144 | 11.919 | 11.340 | 27.808 | 1.00 | 29.44 | N |
| ATOM | 2803 | CA | ALA | L | 144 | 13.274 | 10.826 | 27.955 | 1.00 | 29.42 | C |
| ATOM | 2804 | CB | ALA | L | 144 | 14.021 | 10.973 | 26.660 | 1.00 | 29.11 | C |
| ATOM | 2805 | C | ALA | L | 144 | 14.016 | 11.570 | 29.065 | 1.00 | 29.82 | C |
| ATOM | 2806 | O | ALA | L | 144 | 13.959 | 12.795 | 29.146 | 1.00 | 29.84 | O |
| ATOM | 2807 | N | LYS | L | 145 | 14.718 | 10.826 | 29.912 | 1.00 | 30.30 | N |
| ATOM | 2808 | CA | LYS | L | 145 | 15.539 | 11.424 | 30.958 | 1.00 | 30.84 | C |
| ATOM | 2809 | CB | LYS | L | 145 | 15.177 | 10.829 | 32.332 | 1.00 | 31.25 | C |
| ATOM | 2810 | CG | LYS | L | 145 | 15.865 | 11.486 | 33.535 | 1.00 | 32.82 | C |
| ATOM | 2811 | CD | LYS | L | 145 | 14.939 | 11.559 | 34.768 | 1.00 | 35.76 | C |
| ATOM | 2812 | CE | LYS | L | 145 | 13.852 | 12.648 | 34.584 | 1.00 | 37.62 | C |
| ATOM | 2813 | NZ | LYS | L | 145 | 13.270 | 13.151 | 35.864 | 1.00 | 37.50 | N |
| ATOM | 2814 | C | LYS | L | 145 | 17.029 | 11.261 | 30.623 | 1.00 | 30.70 | C |
| ATOM | 2815 | O | LYS | L | 145 | 17.516 | 10.150 | 30.400 | 1.00 | 30.92 | O |
| ATOM | 2816 | N | VAL | L | 146 | 17.729 | 12.386 | 30.552 | 1.00 | 30.45 | N |
| ATOM | 2817 | CA | VAL | L | 146 | 19.164 | 12.410 | 30.313 | 1.00 | 30.14 | C |
| ATOM | 2818 | CB | VAL | L | 146 | 19.512 | 13.222 | 29.047 | 1.00 | 30.26 | C |
| ATOM | 2819 | CG1 | VAL | L | 146 | 21.021 | 13.341 | 28.875 | 1.00 | 30.34 | C |
| ATOM | 2820 | CG2 | VAL | L | 146 | 18.883 | 12.603 | 27.833 | 1.00 | 29.06 | C |
| ATOM | 2821 | C | VAL | L | 146 | 19.818 | 13.073 | 31.505 | 1.00 | 30.27 | C |
| ATOM | 2822 | O | VAL | L | 146 | 19.479 | 14.206 | 31.844 | 1.00 | 29.82 | O |
| ATOM | 2823 | N | GLN | L | 147 | 20.741 | 12.357 | 32.147 | 1.00 | 30.82 | N |
| ATOM | 2824 | CA | GLN | L | 147 | 21.506 | 12.894 | 33.271 | 1.00 | 31.43 | C |
| ATOM | 2825 | CB | GLN | L | 147 | 21.176 | 12.144 | 34.556 | 1.00 | 31.83 | C |
| ATOM | 2826 | CG | GLN | L | 147 | 19.910 | 12.621 | 35.263 | 1.00 | 33.86 | C |
| ATOM | 2827 | CD | GLN | L | 147 | 19.619 | 11.816 | 36.520 | 1.00 | 36.13 | C |
| ATOM | 2828 | OE1 | GLN | L | 147 | 19.888 | 10.611 | 36.570 | 1.00 | 36.04 | O |
| ATOM | 2829 | NE2 | GLN | L | 147 | 19.072 | 12.478 | 37.546 | 1.00 | 36.06 | N |
| ATOM | 2830 | C | GLN | L | 147 | 23.010 | 12.828 | 33.020 | 1.00 | 31.38 | C |
| ATOM | 2831 | O | GLN | L | 147 | 23.528 | 11.830 | 32.537 | 1.00 | 30.90 | O |
| ATOM | 2832 | N | TRP | L | 148 | 23.705 | 13.901 | 33.369 | 1.00 | 31.86 | N |
| ATOM | 2833 | CA | TRP | L | 148 | 25.147 | 13.957 | 33.225 | 1.00 | 32.02 | C |
| ATOM | 2834 | CB | TRP | L | 148 | 25.581 | 15.336 | 32.757 | 1.00 | 31.70 | C |
| ATOM | 2835 | CG | TRP | L | 148 | 25.335 | 15.623 | 31.305 | 1.00 | 32.08 | C |
| ATOM | 2836 | CD1 | TRP | L | 148 | 24.323 | 16.387 | 30.776 | 1.00 | 32.80 | C |
| ATOM | 2837 | NE1 | TRP | L | 148 | 24.438 | 16.450 | 29.409 | 1.00 | 32.50 | N |
| ATOM | 2838 | CE2 | TRP | L | 148 | 25.541 | 15.733 | 29.022 | 1.00 | 32.26 | C |
| ATOM | 2839 | CD2 | TRP | L | 148 | 26.135 | 15.200 | 30.194 | 1.00 | 32.04 | C |
| ATOM | 2840 | CE3 | TRP | L | 148 | 27.299 | 14.425 | 30.072 | 1.00 | 32.32 | C |
| ATOM | 2841 | CZ3 | TRP | L | 148 | 27.821 | 14.205 | 28.800 | 1.00 | 32.61 | C |
| ATOM | 2842 | CH2 | TRP | L | 148 | 27.202 | 14.750 | 27.652 | 1.00 | 31.99 | C |
| ATOM | 2843 | CZ2 | TRP | L | 148 | 26.067 | 15.512 | 27.744 | 1.00 | 31.82 | C |
| ATOM | 2844 | C | TRP | L | 148 | 25.845 | 13.611 | 34.534 | 1.00 | 32.46 | C |
| ATOM | 2845 | O | TRP | L | 148 | 25.440 | 14.042 | 35.614 | 1.00 | 32.32 | O |
| ATOM | 2846 | N | LYS | L | 149 | 26.900 | 12.819 | 34.421 | 1.00 | 33.21 | N |
| ATOM | 2847 | CA | LYS | L | 149 | 27.711 | 12.433 | 35.559 | 1.00 | 34.15 | C |
| ATOM | 2848 | CB | LYS | L | 149 | 27.501 | 10.951 | 35.903 | 1.00 | 34.39 | C |
| ATOM | 2849 | CG | LYS | L | 149 | 26.527 | 10.731 | 37.059 | 1.00 | 36.56 | C |
| ATOM | 2850 | CD | LYS | L | 149 | 25.739 | 9.428 | 36.943 | 1.00 | 39.81 | C |
| ATOM | 2851 | CE | LYS | L | 149 | 24.935 | 9.160 | 38.229 | 1.00 | 41.17 | C |
| ATOM | 2852 | NZ | LYS | L | 149 | 23.746 | 8.290 | 37.992 | 1.00 | 41.58 | N |
| ATOM | 2853 | C | LYS | L | 149 | 29.162 | 12.726 | 35.236 | 1.00 | 34.27 | C |
| ATOM | 2854 | O | LYS | L | 149 | 29.720 | 12.179 | 34.275 | 1.00 | 34.22 | O |
| ATOM | 2855 | N | VAL | L | 150 | 29.752 | 13.624 | 36.017 | 1.00 | 34.51 | N |
| ATOM | 2856 | CA | VAL | L | 150 | 31.167 | 13.935 | 35.883 | 1.00 | 35.00 | C |
| ATOM | 2857 | CB | VAL | L | 150 | 31.417 | 15.448 | 35.683 | 1.00 | 34.81 | C |
| ATOM | 2858 | CG1 | VAL | L | 150 | 32.897 | 15.730 | 35.536 | 1.00 | 34.70 | C |
| ATOM | 2859 | CG2 | VAL | L | 150 | 30.691 | 15.941 | 34.451 | 1.00 | 34.19 | C |
| ATOM | 2860 | C | VAL | L | 150 | 31.898 | 13.389 | 37.109 | 1.00 | 35.65 | C |
| ATOM | 2861 | O | VAL | L | 150 | 31.762 | 13.931 | 38.210 | 1.00 | 35.95 | O |
| ATOM | 2862 | N | ASP | L | 151 | 32.655 | 12.305 | 36.903 | 1.00 | 36.20 | N |
| ATOM | 2863 | CA | ASP | L | 151 | 33.309 | 11.554 | 37.984 | 1.00 | 36.80 | C |
| ATOM | 2864 | CB | ASP | L | 151 | 34.428 | 12.371 | 38.675 | 1.00 | 36.75 | C |
| ATOM | 2865 | CG | ASP | L | 151 | 35.732 | 12.418 | 37.859 | 1.00 | 36.72 | C |
| ATOM | 2866 | OD1 | ASP | L | 151 | 36.103 | 11.416 | 37.207 | 1.00 | 36.35 | O |
| ATOM | 2867 | OD2 | ASP | L | 151 | 36.405 | 13.463 | 37.888 | 1.00 | 36.44 | O |
| ATOM | 2868 | C | ASP | L | 151 | 32.253 | 11.091 | 38.988 | 1.00 | 37.29 | C |
| ATOM | 2869 | O | ASP | L | 151 | 32.475 | 11.102 | 40.204 | 1.00 | 37.72 | O |
| ATOM | 2870 | N | ASN | L | 152 | 31.096 | 10.702 | 38.453 | 1.00 | 37.46 | N |
| ATOM | 2871 | CA | ASN | L | 152 | 29.960 | 10.218 | 39.238 | 1.00 | 37.13 | C |
| ATOM | 2872 | CB | ASN | L | 152 | 30.365 | 9.048 | 40.132 | 1.00 | 37.65 | C |
| ATOM | 2873 | CG | ASN | L | 152 | 30.082 | 7.720 | 39.484 | 1.00 | 39.11 | C |
| ATOM | 2874 | OD1 | ASN | L | 152 | 28.931 | 7.273 | 39.436 | 1.00 | 40.28 | O |
| ATOM | 2875 | ND2 | ASN | L | 152 | 31.129 | 7.079 | 38.962 | 1.00 | 40.96 | N |
| ATOM | 2876 | C | ASN | L | 152 | 29.175 | 11.249 | 40.031 | 1.00 | 36.47 | C |
| ATOM | 2877 | O | ASN | L | 152 | 28.195 | 10.899 | 40.688 | 1.00 | 36.68 | O |
| ATOM | 2878 | N | ALA | L | 153 | 29.590 | 12.511 | 39.972 | 1.00 | 35.55 | N |
| ATOM | 2879 | CA | ALA | L | 153 | 28.790 | 13.586 | 40.545 | 1.00 | 34.77 | C |
| ATOM | 2880 | CB | ALA | L | 153 | 29.664 | 14.763 | 40.909 | 1.00 | 34.63 | C |
| ATOM | 2881 | C | ALA | L | 153 | 27.711 | 13.999 | 39.542 | 1.00 | 34.52 | C |
| ATOM | 2882 | O | ALA | L | 153 | 28.015 | 14.305 | 38.378 | 1.00 | 34.56 | O |
| ATOM | 2883 | N | LEU | L | 154 | 26.453 | 13.994 | 39.984 | 1.00 | 33.70 | N |
| ATOM | 2884 | CA | LEU | L | 154 | 25.348 | 14.446 | 39.135 | 1.00 | 32.87 | C |
| ATOM | 2885 | CB | LEU | L | 154 | 23.969 | 14.076 | 39.719 | 1.00 | 33.04 | C |
| ATOM | 2886 | CG | LEU | L | 154 | 22.739 | 14.737 | 39.049 | 1.00 | 33.71 | C |
| ATOM | 2887 | CD1 | LEU | L | 154 | 22.532 | 14.310 | 37.568 | 1.00 | 33.20 | C |
| ATOM | 2888 | CD2 | LEU | L | 154 | 21.451 | 14.523 | 39.868 | 1.00 | 33.64 | C |
| ATOM | 2889 | C | LEU | L | 154 | 25.433 | 15.941 | 38.876 | 1.00 | 31.93 | C |
| ATOM | 2890 | O | LEU | L | 154 | 25.554 | 16.746 | 39.799 | 1.00 | 32.13 | O |
| ATOM | 2891 | N | GLN | L | 155 | 25.358 | 16.297 | 37.602 | 1.00 | 30.91 | N |
| ATOM | 2892 | CA | GLN | L | 155 | 25.421 | 17.686 | 37.179 | 1.00 | 29.79 | C |
| ATOM | 2893 | CB | GLN | L | 155 | 26.056 | 17.772 | 35.792 | 1.00 | 29.59 | C |
| ATOM | 2894 | CG | GLN | L | 155 | 27.458 | 17.159 | 35.716 | 1.00 | 29.67 | C |
| ATOM | 2895 | CD | GLN | L | 155 | 28.517 | 17.999 | 36.440 | 1.00 | 30.45 | C |
| ATOM | 2896 | OE1 | GLN | L | 155 | 28.772 | 19.151 | 36.077 | 1.00 | 30.02 | O |
| ATOM | 2897 | NE2 | GLN | L | 155 | 29.136 | 17.419 | 37.464 | 1.00 | 30.33 | N |
| ATOM | 2898 | C | GLN | L | 155 | 24.031 | 18.305 | 37.160 | 1.00 | 29.09 | C |
| ATOM | 2899 | O | GLN | L | 155 | 23.043 | 17.614 | 36.906 | 1.00 | 29.16 | O |
| ATOM | 2900 | N | SER | L | 156 | 23.951 | 19.598 | 37.457 | 1.00 | 28.07 | N |
| ATOM | 2901 | CA | SER | L | 156 | 22.752 | 20.350 | 37.145 | 1.00 | 27.35 | C |
| ATOM | 2902 | CB | SER | L | 156 | 21.670 | 20.237 | 38.242 | 1.00 | 27.75 | C |
| ATOM | 2903 | OG | SER | L | 156 | 22.180 | 20.400 | 39.544 | 1.00 | 26.91 | O |
| ATOM | 2904 | C | SER | L | 156 | 23.046 | 21.787 | 36.769 | 1.00 | 26.86 | C |
| ATOM | 2905 | O | SER | L | 156 | 24.007 | 22.380 | 37.246 | 1.00 | 27.17 | O |
| ATOM | 2906 | N | GLY | L | 157 | 22.218 | 22.316 | 35.880 | 1.00 | 26.39 | N |
| ATOM | 2907 | CA | GLY | L | 157 | 22.274 | 23.706 | 35.467 | 1.00 | 26.48 | C |
| ATOM | 2908 | C | GLY | L | 157 | 23.357 | 24.053 | 34.467 | 1.00 | 26.61 | C |
| ATOM | 2909 | O | GLY | L | 157 | 23.450 | 25.203 | 34.052 | 1.00 | 26.40 | O |
| ATOM | 2910 | N | ASN | L | 158 | 24.177 | 23.073 | 34.079 | 1.00 | 26.63 | N |
| ATOM | 2911 | CA | ASN | L | 158 | 25.244 | 23.308 | 33.101 | 1.00 | 26.67 | C |
| ATOM | 2912 | CB | ASN | L | 158 | 26.622 | 23.035 | 33.722 | 1.00 | 26.76 | C |
| ATOM | 2913 | CG | ASN | L | 158 | 26.742 | 21.622 | 34.280 | 1.00 | 28.06 | C |
| ATOM | 2914 | OD1 | ASN | L | 158 | 25.768 | 20.866 | 34.300 | 1.00 | 29.54 | O |
| ATOM | 2915 | ND2 | ASN | L | 158 | 27.943 | 21.255 | 34.725 | 1.00 | 27.96 | N |
| ATOM | 2916 | C | ASN | L | 158 | 25.059 | 22.526 | 31.792 | 1.00 | 26.32 | C |
| ATOM | 2917 | O | ASN | L | 158 | 26.012 | 22.327 | 31.040 | 1.00 | 26.44 | O |
| ATOM | 2918 | N | SER | L | 159 | 23.832 | 22.085 | 31.529 | 1.00 | 26.03 | N |
| ATOM | 2919 | CA | SER | L | 159 | 23.508 | 21.373 | 30.297 | 1.00 | 25.82 | C |
| ATOM | 2920 | CB | SER | L | 159 | 23.304 | 19.881 | 30.551 | 1.00 | 25.80 | C |
| ATOM | 2921 | OG | SER | L | 159 | 22.248 | 19.661 | 31.463 | 1.00 | 25.10 | O |
| ATOM | 2922 | C | SER | L | 159 | 22.271 | 21.952 | 29.662 | 1.00 | 25.94 | C |
| ATOM | 2923 | O | SER | L | 159 | 21.425 | 22.505 | 30.346 | 1.00 | 26.59 | O |
| ATOM | 2924 | N | GLN | L | 160 | 22.184 | 21.844 | 28.345 | 1.00 | 26.32 | N |
| ATOM | 2925 | CA | GLN | L | 160 | 20.993 | 22.248 | 27.621 | 1.00 | 26.66 | C |
| ATOM | 2926 | CB | GLN | L | 160 | 21.171 | 23.627 | 26.985 | 1.00 | 26.76 | C |
| ATOM | 2927 | CG | GLN | L | 160 | 21.114 | 24.758 | 28.030 | 1.00 | 28.16 | C |
| ATOM | 2928 | CD | GLN | L | 160 | 21.351 | 26.150 | 27.463 | 1.00 | 29.85 | C |
| ATOM | 2929 | OE1 | GLN | L | 160 | 22.383 | 26.417 | 26.838 | 1.00 | 31.69 | O |
| ATOM | 2930 | NE2 | GLN | L | 160 | 20.410 | 27.056 | 27.711 | 1.00 | 28.19 | N |
| ATOM | 2931 | C | GLN | L | 160 | 20.588 | 21.190 | 26.606 | 1.00 | 26.57 | C |
| ATOM | 2932 | O | GLN | L | 160 | 21.437 | 20.553 | 25.988 | 1.00 | 26.90 | O |
| ATOM | 2933 | N | GLU | L | 161 | 19.273 | 21.013 | 26.478 | 1.00 | 26.36 | N |
| ATOM | 2934 | CA | GLU | L | 161 | 18.627 | 20.015 | 25.635 | 1.00 | 25.74 | C |
| ATOM | 2935 | CB | GLU | L | 161 | 17.576 | 19.289 | 26.456 | 1.00 | 25.62 | C |
| ATOM | 2936 | CG | GLU | L | 161 | 18.007 | 18.048 | 27.144 | 1.00 | 26.71 | C |
| ATOM | 2937 | CD | GLU | L | 161 | 16.811 | 17.290 | 27.668 | 1.00 | 27.78 | C |
| ATOM | 2938 | OE1 | GLU | L | 161 | 17.007 | 16.354 | 28.471 | 1.00 | 29.34 | O |
| ATOM | 2939 | OE2 | GLU | L | 161 | 15.670 | 17.633 | 27.273 | 1.00 | 27.54 | O |
| ATOM | 2940 | C | GLU | L | 161 | 17.827 | 20.666 | 24.526 | 1.00 | 25.21 | C |
| ATOM | 2941 | O | GLU | L | 161 | 17.149 | 21.673 | 24.755 | 1.00 | 25.14 | O |
| ATOM | 2942 | N | SER | L | 162 | 17.851 | 20.063 | 23.345 | 1.00 | 24.17 | N |
| ATOM | 2943 | CA | SER | L | 162 | 16.799 | 20.328 | 22.381 | 1.00 | 23.38 | C |
| ATOM | 2944 | CB | SER | L | 162 | 17.116 | 21.533 | 21.475 | 1.00 | 23.42 | C |
| ATOM | 2945 | OG | SER | L | 162 | 18.010 | 21.206 | 20.436 | 1.00 | 23.35 | O |
| ATOM | 2946 | C | SER | L | 162 | 16.424 | 19.057 | 21.621 | 1.00 | 22.72 | C |
| ATOM | 2947 | O | SER | L | 162 | 17.186 | 18.091 | 21.575 | 1.00 | 22.05 | O |
| ATOM | 2948 | N | VAL | L | 163 | 15.222 | 19.071 | 21.062 | 1.00 | 22.34 | N |
| ATOM | 2949 | CA | VAL | L | 163 | 14.593 | 17.883 | 20.514 | 1.00 | 22.55 | C |
| ATOM | 2950 | CB | VAL | L | 163 | 13.475 | 17.358 | 21.473 | 1.00 | 22.43 | C |
| ATOM | 2951 | CG1 | VAL | L | 163 | 12.335 | 18.377 | 21.589 | 1.00 | 23.00 | C |
| ATOM | 2952 | CG2 | VAL | L | 163 | 12.937 | 16.005 | 21.013 | 1.00 | 22.44 | C |
| ATOM | 2953 | C | VAL | L | 163 | 14.034 | 18.206 | 19.133 | 1.00 | 22.69 | C |
| ATOM | 2954 | O | VAL | L | 163 | 13.439 | 19.270 | 18.929 | 1.00 | 23.29 | O |
| ATOM | 2955 | N | THR | L | 164 | 14.234 | 17.305 | 18.180 | 1.00 | 22.58 | N |
| ATOM | 2956 | CA | THR | L | 164 | 13.723 | 17.525 | 16.822 | 1.00 | 22.65 | C |
| ATOM | 2957 | CB | THR | L | 164 | 14.352 | 16.540 | 15.802 | 1.00 | 22.48 | C |
| ATOM | 2958 | OG1 | THR | L | 164 | 14.248 | 15.198 | 16.301 | 1.00 | 21.70 | O |
| ATOM | 2959 | CG2 | THR | L | 164 | 15.810 | 16.879 | 15.542 | 1.00 | 21.78 | C |
| ATOM | 2960 | C | THR | L | 164 | 12.205 | 17.388 | 16.770 | 1.00 | 23.07 | C |
| ATOM | 2961 | O | THR | L | 164 | 11.592 | 16.897 | 17.705 | 1.00 | 23.36 | O |
| ATOM | 2962 | N | GLU | L | 165 | 11.599 | 17.830 | 15.677 | 1.00 | 23.96 | N |
| ATOM | 2963 | CA | GLU | L | 165 | 10.194 | 17.525 | 15.417 | 1.00 | 24.80 | C |
| ATOM | 2964 | CB | GLU | L | 165 | 9.626 | 18.423 | 14.308 | 1.00 | 24.87 | C |
| ATOM | 2965 | CG | GLU | L | 165 | 9.475 | 19.895 | 14.701 | 1.00 | 28.31 | C |
| ATOM | 2966 | CD | GLU | L | 165 | 8.413 | 20.127 | 15.787 | 1.00 | 33.35 | C |
| ATOM | 2967 | OE1 | GLU | L | 165 | 7.225 | 19.785 | 15.557 | 1.00 | 35.24 | O |
| ATOM | 2968 | OE2 | GLU | L | 165 | 8.767 | 20.653 | 16.873 | 1.00 | 35.13 | O |
| ATOM | 2969 | C | GLU | L | 165 | 10.069 | 16.050 | 15.032 | 1.00 | 24.61 | C |
| ATOM | 2970 | O | GLU | L | 165 | 11.021 | 15.459 | 14.494 | 1.00 | 24.72 | O |
| ATOM | 2971 | N | GLN | L | 166 | 8.912 | 15.458 | 15.324 | 1.00 | 24.18 | N |
| ATOM | 2972 | CA | GLN | L | 166 | 8.600 | 14.096 | 14.891 | 1.00 | 24.01 | C |
| ATOM | 2973 | CB | GLN | L | 166 | 7.131 | 13.764 | 15.180 | 1.00 | 23.62 | C |
| ATOM | 2974 | CG | GLN | L | 166 | 6.723 | 12.344 | 14.825 | 1.00 | 23.27 | C |
| ATOM | 2975 | CD | GLN | L | 166 | 5.473 | 11.860 | 15.568 | 1.00 | 22.24 | C |
| ATOM | 2976 | OE1 | GLN | L | 166 | 4.413 | 12.482 | 15.513 | 1.00 | 18.91 | O |
| ATOM | 2977 | NE2 | GLN | L | 166 | 5.602 | 10.726 | 16.249 | 1.00 | 21.32 | N |
| ATOM | 2978 | C | GLN | L | 166 | 8.922 | 13.920 | 13.405 | 1.00 | 24.32 | C |
| ATOM | 2979 | O | GLN | L | 166 | 8.486 | 14.708 | 12.567 | 1.00 | 24.26 | O |
| ATOM | 2980 | N | ASP | L | 167 | 9.712 | 12.898 | 13.107 | 1.00 | 24.79 | N |
| ATOM | 2981 | CA | ASP | L | 167 | 10.114 | 12.560 | 11.756 | 1.00 | 25.93 | C |
| ATOM | 2982 | CB | ASP | L | 167 | 11.204 | 11.486 | 11.809 | 1.00 | 25.80 | C |
| ATOM | 2983 | CG | ASP | L | 167 | 11.654 | 11.038 | 10.434 | 1.00 | 26.84 | C |
| ATOM | 2984 | OD1 | ASP | L | 167 | 11.230 | 9.946 | 9.988 | 1.00 | 28.64 | O |
| ATOM | 2985 | OD2 | ASP | L | 167 | 12.428 | 11.778 | 9.798 | 1.00 | 27.71 | O |
| ATOM | 2986 | C | ASP | L | 167 | 8.904 | 12.041 | 10.981 | 1.00 | 26.68 | C |
| ATOM | 2987 | O | ASP | L | 167 | 8.165 | 11.197 | 11.484 | 1.00 | 26.55 | O |
| ATOM | 2988 | N | SER | L | 168 | 8.715 | 12.536 | 9.756 | 1.00 | 27.79 | N |
| ATOM | 2989 | CA | SER | L | 168 | 7.485 | 12.248 | 9.007 | 1.00 | 28.89 | C |
| ATOM | 2990 | CB | SER | L | 168 | 7.262 | 13.233 | 7.846 | 1.00 | 29.08 | C |
| ATOM | 2991 | OG | SER | L | 168 | 8.473 | 13.527 | 7.171 | 1.00 | 30.33 | O |
| ATOM | 2992 | C | SER | L | 168 | 7.370 | 10.795 | 8.547 | 1.00 | 29.14 | C |
| ATOM | 2993 | O | SER | L | 168 | 6.273 | 10.249 | 8.520 | 1.00 | 29.53 | O |
| ATOM | 2994 | N | LYS | L | 169 | 8.498 | 10.163 | 8.238 | 1.00 | 29.72 | N |
| ATOM | 2995 | CA | LYS | L | 169 | 8.493 | 8.778 | 7.743 | 1.00 | 29.82 | C |
| ATOM | 2996 | CB | LYS | L | 169 | 9.599 | 8.550 | 6.710 | 1.00 | 30.56 | C |
| ATOM | 2997 | CG | LYS | L | 169 | 9.285 | 9.146 | 5.319 | 1.00 | 34.03 | C |
| ATOM | 2998 | CD | LYS | L | 169 | 10.510 | 9.044 | 4.391 | 1.00 | 38.52 | C |
| ATOM | 2999 | CE | LYS | L | 169 | 10.846 | 10.404 | 3.777 | 1.00 | 40.15 | C |
| ATOM | 3000 | NZ | LYS | L | 169 | 12.321 | 10.590 | 3.670 | 1.00 | 41.61 | N |
| ATOM | 3001 | C | LYS | L | 169 | 8.570 | 7.708 | 8.820 | 1.00 | 28.95 | C |
| ATOM | 3002 | O | LYS | L | 169 | 7.938 | 6.658 | 8.669 | 1.00 | 29.14 | O |
| ATOM | 3003 | N | ASP | L | 170 | 9.333 | 7.945 | 9.892 | 1.00 | 27.63 | N |
| ATOM | 3004 | CA | ASP | L | 170 | 9.429 | 6.926 | 10.957 | 1.00 | 26.53 | C |
| ATOM | 3005 | CB | ASP | L | 170 | 10.865 | 6.373 | 11.111 | 1.00 | 26.65 | C |
| ATOM | 3006 | CG | ASP | L | 170 | 11.796 | 7.293 | 11.899 | 1.00 | 27.41 | C |
| ATOM | 3007 | OD1 | ASP | L | 170 | 11.346 | 8.281 | 12.525 | 1.00 | 27.60 | O |
| ATOM | 3008 | OD2 | ASP | L | 170 | 13.008 | 6.997 | 11.910 | 1.00 | 28.71 | O |
| ATOM | 3009 | C | ASP | L | 170 | 8.801 | 7.264 | 12.313 | 1.00 | 25.59 | C |
| ATOM | 3010 | O | ASP | L | 170 | 8.739 | 6.412 | 13.199 | 1.00 | 25.43 | O |
| ATOM | 3011 | N | SER | L | 171 | 8.356 | 8.511 | 12.462 | 1.00 | 24.48 | N |
| ATOM | 3012 | CA | SER | L | 171 | 7.656 | 8.992 | 13.661 | 1.00 | 23.55 | C |
| ATOM | 3013 | CB | SER | L | 171 | 6.299 | 8.286 | 13.831 | 1.00 | 23.66 | C |
| ATOM | 3014 | OG | SER | L | 171 | 5.528 | 8.356 | 12.636 | 1.00 | 24.38 | O |
| ATOM | 3015 | C | SER | L | 171 | 8.471 | 8.946 | 14.963 | 1.00 | 22.72 | C |
| ATOM | 3016 | O | SER | L | 171 | 7.909 | 8.877 | 16.054 | 1.00 | 23.35 | O |
| ATOM | 3017 | N | THR | L | 172 | 9.788 | 9.003 | 14.858 | 1.00 | 21.31 | N |
| ATOM | 3018 | CA | THR | L | 172 | 10.628 | 9.020 | 16.046 | 1.00 | 19.74 | C |
| ATOM | 3019 | CB | THR | L | 172 | 11.871 | 8.131 | 15.868 | 1.00 | 19.68 | C |
| ATOM | 3020 | OG1 | THR | L | 172 | 12.635 | 8.598 | 14.757 | 1.00 | 19.30 | O |
| ATOM | 3021 | CG2 | THR | L | 172 | 11.489 | 6.677 | 15.633 | 1.00 | 18.87 | C |
| ATOM | 3022 | C | THR | L | 172 | 11.096 | 10.440 | 16.349 | 1.00 | 18.92 | C |
| ATOM | 3023 | O | THR | L | 172 | 11.042 | 11.310 | 15.493 | 1.00 | 18.26 | O |
| ATOM | 3024 | N | TYR | L | 173 | 11.548 | 10.657 | 17.575 | 1.00 | 18.16 | N |
| ATOM | 3025 | CA | TYR | L | 173 | 12.198 | 11.903 | 17.963 | 1.00 | 17.83 | C |
| ATOM | 3026 | CB | TYR | L | 173 | 11.624 | 12.433 | 19.288 | 1.00 | 17.90 | C |
| ATOM | 3027 | CG | TYR | L | 173 | 10.163 | 12.804 | 19.208 | 1.00 | 19.58 | C |
| ATOM | 3028 | CD1 | TYR | L | 173 | 9.174 | 11.850 | 19.450 | 1.00 | 20.74 | C |
| ATOM | 3029 | CE1 | TYR | L | 173 | 7.831 | 12.174 | 19.367 | 1.00 | 21.71 | C |
| ATOM | 3030 | CZ | TYR | L | 173 | 7.463 | 13.463 | 19.044 | 1.00 | 23.12 | C |
| ATOM | 3031 | OH | TYR | L | 173 | 6.128 | 13.763 | 18.968 | 1.00 | 25.14 | O |
| ATOM | 3032 | CE2 | TYR | L | 173 | 8.424 | 14.443 | 18.802 | 1.00 | 21.75 | C |
| ATOM | 3033 | CD2 | TYR | L | 173 | 9.766 | 14.106 | 18.878 | 1.00 | 19.41 | C |
| ATOM | 3034 | C | TYR | L | 173 | 13.683 | 11.655 | 18.145 | 1.00 | 17.41 | C |
| ATOM | 3035 | O | TYR | L | 173 | 14.109 | 10.525 | 18.418 | 1.00 | 16.41 | O |
| ATOM | 3036 | N | SER | L | 174 | 14.459 | 12.722 | 18.011 | 1.00 | 17.04 | N |
| ATOM | 3037 | CA | SER | L | 174 | 15.845 | 12.688 | 18.391 | 1.00 | 17.37 | C |
| ATOM | 3038 | CB | SER | L | 174 | 16.743 | 12.749 | 17.164 | 1.00 | 17.29 | C |
| ATOM | 3039 | OG | SER | L | 174 | 16.937 | 11.435 | 16.659 | 1.00 | 19.15 | O |
| ATOM | 3040 | C | SER | L | 174 | 16.127 | 13.822 | 19.345 | 1.00 | 17.74 | C |
| ATOM | 3041 | O | SER | L | 174 | 15.382 | 14.808 | 19.368 | 1.00 | 17.79 | O |
| ATOM | 3042 | N | LEU | L | 175 | 17.199 | 13.686 | 20.132 | 1.00 | 18.03 | N |
| ATOM | 3043 | CA | LEU | L | 175 | 17.544 | 14.685 | 21.152 | 1.00 | 18.01 | C |
| ATOM | 3044 | CB | LEU | L | 175 | 16.920 | 14.288 | 22.493 | 1.00 | 17.48 | C |
| ATOM | 3045 | CG | LEU | L | 175 | 17.017 | 15.223 | 23.696 | 1.00 | 18.38 | C |
| ATOM | 3046 | CD1 | LEU | L | 175 | 15.849 | 14.965 | 24.659 | 1.00 | 18.71 | C |
| ATOM | 3047 | CD2 | LEU | L | 175 | 18.352 | 15.041 | 24.423 | 1.00 | 18.95 | C |
| ATOM | 3048 | C | LEU | L | 175 | 19.055 | 14.884 | 21.311 | 1.00 | 18.11 | C |
| ATOM | 3049 | O | LEU | L | 175 | 19.833 | 13.944 | 21.242 | 1.00 | 17.50 | O |
| ATOM | 3050 | N | SER | L | 176 | 19.466 | 16.117 | 21.554 | 1.00 | 18.60 | N |
| ATOM | 3051 | CA | SER | L | 176 | 20.849 | 16.357 | 21.898 | 1.00 | 19.44 | C |
| ATOM | 3052 | CB | SER | L | 176 | 21.551 | 17.207 | 20.841 | 1.00 | 18.96 | C |
| ATOM | 3053 | OG | SER | L | 176 | 20.999 | 18.498 | 20.861 | 1.00 | 20.43 | O |
| ATOM | 3054 | C | SER | L | 176 | 20.908 | 17.062 | 23.229 | 1.00 | 19.71 | C |
| ATOM | 3055 | O | SER | L | 176 | 20.208 | 18.052 | 23.450 | 1.00 | 20.04 | O |
| ATOM | 3056 | N | SER | L | 177 | 21.759 | 16.550 | 24.105 | 1.00 | 19.99 | N |
| ATOM | 3057 | CA | SER | L | 177 | 22.034 | 17.210 | 25.363 | 1.00 | 20.82 | C |
| ATOM | 3058 | CB | SER | L | 177 | 21.807 | 16.250 | 26.534 | 1.00 | 20.60 | C |
| ATOM | 3059 | OG | SER | L | 177 | 22.204 | 16.864 | 27.740 | 1.00 | 21.29 | O |
| ATOM | 3060 | C | SER | L | 177 | 23.465 | 17.697 | 25.352 | 1.00 | 21.15 | C |
| ATOM | 3061 | O | SER | L | 177 | 24.378 | 16.926 | 25.059 | 1.00 | 21.68 | O |
| ATOM | 3062 | N | THR | L | 178 | 23.665 | 18.971 | 25.664 | 1.00 | 21.64 | N |
| ATOM | 3063 | CA | THR | L | 178 | 25.004 | 19.536 | 25.672 | 1.00 | 22.39 | C |
| ATOM | 3064 | CB | THR | L | 178 | 25.163 | 20.737 | 24.687 | 1.00 | 22.32 | C |
| ATOM | 3065 | OG1 | THR | L | 178 | 24.822 | 20.325 | 23.359 | 1.00 | 23.00 | O |
| ATOM | 3066 | CG2 | THR | L | 178 | 26.614 | 21.265 | 24.688 | 1.00 | 22.03 | C |
| ATOM | 3067 | C | THR | L | 178 | 25.420 | 19.974 | 27.063 | 1.00 | 23.07 | C |
| ATOM | 3068 | O | THR | L | 178 | 24.809 | 20.868 | 27.657 | 1.00 | 23.27 | O |
| ATOM | 3069 | N | LEU | L | 179 | 26.476 | 19.338 | 27.560 | 1.00 | 23.74 | N |
| ATOM | 3070 | CA | LEU | L | 179 | 27.176 | 19.763 | 28.761 | 1.00 | 24.41 | C |
| ATOM | 3071 | CB | LEU | L | 179 | 27.839 | 18.541 | 29.408 | 1.00 | 24.20 | C |
| ATOM | 3072 | CG | LEU | L | 179 | 28.639 | 18.642 | 30.715 | 1.00 | 24.11 | C |
| ATOM | 3073 | CD1 | LEU | L | 179 | 27.802 | 19.210 | 31.863 | 1.00 | 24.08 | C |
| ATOM | 3074 | CD2 | LEU | L | 179 | 29.161 | 17.265 | 31.085 | 1.00 | 23.04 | C |
| ATOM | 3075 | C | LEU | L | 179 | 28.226 | 20.802 | 28.359 | 1.00 | 25.28 | C |
| ATOM | 3076 | O | LEU | L | 179 | 29.075 | 20.534 | 27.507 | 1.00 | 25.45 | O |
| ATOM | 3077 | N | THR | L | 180 | 28.160 | 21.982 | 28.960 | 1.00 | 26.45 | N |
| ATOM | 3078 | CA | THR | L | 180 | 29.120 | 23.052 | 28.683 | 1.00 | 28.16 | C |
| ATOM | 3079 | CB | THR | L | 180 | 28.413 | 24.321 | 28.196 | 1.00 | 28.13 | C |
| ATOM | 3080 | OG1 | THR | L | 180 | 27.605 | 24.022 | 27.052 | 1.00 | 28.30 | O |
| ATOM | 3081 | CG2 | THR | L | 180 | 29.430 | 25.410 | 27.856 | 1.00 | 28.60 | C |
| ATOM | 3082 | C | THR | L | 180 | 29.903 | 23.410 | 29.948 | 1.00 | 29.28 | C |
| ATOM | 3083 | O | THR | L | 180 | 29.307 | 23.657 | 31.001 | 1.00 | 29.47 | O |
| ATOM | 3084 | N | LEU | L | 181 | 31.232 | 23.434 | 29.833 | 1.00 | 30.45 | N |
| ATOM | 3085 | CA | LEU | L | 181 | 32.141 | 23.753 | 30.947 | 1.00 | 31.42 | C |
| ATOM | 3086 | CB | LEU | L | 181 | 32.766 | 22.478 | 31.521 | 1.00 | 31.06 | C |
| ATOM | 3087 | CG | LEU | L | 181 | 31.931 | 21.224 | 31.774 | 1.00 | 31.26 | C |
| ATOM | 3088 | CD1 | LEU | L | 181 | 32.777 | 19.987 | 31.537 | 1.00 | 30.85 | C |
| ATOM | 3089 | CD2 | LEU | L | 181 | 31.338 | 21.207 | 33.175 | 1.00 | 31.59 | C |
| ATOM | 3090 | C | LEU | L | 181 | 33.264 | 24.664 | 30.447 | 1.00 | 32.17 | C |
| ATOM | 3091 | O | LEU | L | 181 | 33.438 | 24.839 | 29.244 | 1.00 | 32.57 | O |
| ATOM | 3092 | N | SER | L | 182 | 34.038 | 25.235 | 31.359 | 1.00 | 33.44 | N |
| ATOM | 3093 | CA | SER | L | 182 | 35.265 | 25.937 | 30.965 | 1.00 | 34.73 | C |
| ATOM | 3094 | CB | SER | L | 182 | 35.693 | 26.942 | 32.044 | 1.00 | 34.85 | C |
| ATOM | 3095 | OG | SER | L | 182 | 36.099 | 26.279 | 33.239 | 1.00 | 34.91 | O |
| ATOM | 3096 | C | SER | L | 182 | 36.337 | 24.871 | 30.756 | 1.00 | 35.29 | C |
| ATOM | 3097 | O | SER | L | 182 | 36.238 | 23.795 | 31.345 | 1.00 | 35.23 | O |
| ATOM | 3098 | N | LYS | L | 183 | 37.340 | 25.154 | 29.919 | 1.00 | 36.52 | N |
| ATOM | 3099 | CA | LYS | L | 183 | 38.479 | 24.232 | 29.730 | 1.00 | 37.56 | C |
| ATOM | 3100 | CB | LYS | L | 183 | 39.591 | 24.866 | 28.879 | 1.00 | 37.95 | C |
| ATOM | 3101 | CG | LYS | L | 183 | 40.738 | 23.902 | 28.542 | 1.00 | 38.88 | C |
| ATOM | 3102 | CD | LYS | L | 183 | 42.075 | 24.626 | 28.336 | 1.00 | 41.34 | C |
| ATOM | 3103 | CE | LYS | L | 183 | 43.280 | 23.743 | 28.744 | 1.00 | 41.89 | C |
| ATOM | 3104 | NZ | LYS | L | 183 | 43.291 | 23.338 | 30.200 | 1.00 | 40.39 | N |
| ATOM | 3105 | C | LYS | L | 183 | 39.046 | 23.802 | 31.085 | 1.00 | 37.95 | C |
| ATOM | 3106 | O | LYS | L | 183 | 39.247 | 22.604 | 31.338 | 1.00 | 37.55 | O |
| ATOM | 3107 | N | ALA | L | 184 | 39.275 | 24.796 | 31.949 | 1.00 | 38.59 | N |
| ATOM | 3108 | CA | ALA | L | 184 | 39.750 | 24.585 | 33.315 | 1.00 | 39.40 | C |
| ATOM | 3109 | CB | ALA | L | 184 | 39.781 | 25.914 | 34.075 | 1.00 | 39.31 | C |
| ATOM | 3110 | C | ALA | L | 184 | 38.904 | 23.542 | 34.058 | 1.00 | 39.92 | C |
| ATOM | 3111 | O | ALA | L | 184 | 39.396 | 22.442 | 34.375 | 1.00 | 40.14 | O |
| ATOM | 3112 | N | ASP | L | 185 | 37.632 | 23.883 | 34.296 | 1.00 | 40.60 | N |
| ATOM | 3113 | CA | ASP | L | 185 | 36.670 | 22.989 | 34.960 | 1.00 | 41.08 | C |
| ATOM | 3114 | CB | ASP | L | 185 | 35.271 | 23.616 | 35.011 | 1.00 | 41.41 | C |
| ATOM | 3115 | CG | ASP | L | 185 | 35.070 | 24.498 | 36.242 | 1.00 | 42.61 | C |
| ATOM | 3116 | OD1 | ASP | L | 185 | 35.474 | 24.058 | 37.342 | 1.00 | 43.73 | O |
| ATOM | 3117 | OD2 | ASP | L | 185 | 34.502 | 25.615 | 36.120 | 1.00 | 42.57 | O |
| ATOM | 3118 | C | ASP | L | 185 | 36.607 | 21.626 | 34.304 | 1.00 | 40.94 | C |
| ATOM | 3119 | O | ASP | L | 185 | 36.520 | 20.609 | 34.996 | 1.00 | 41.33 | O |
| ATOM | 3120 | N | TYR | L | 186 | 36.664 | 21.609 | 32.972 | 1.00 | 40.62 | N |
| ATOM | 3121 | CA | TYR | L | 186 | 36.718 | 20.358 | 32.220 | 1.00 | 40.36 | C |
| ATOM | 3122 | CB | TYR | L | 186 | 36.690 | 20.621 | 30.703 | 1.00 | 39.49 | C |
| ATOM | 3123 | CG | TYR | L | 186 | 36.798 | 19.372 | 29.851 | 1.00 | 36.02 | C |
| ATOM | 3124 | CD1 | TYR | L | 186 | 35.830 | 18.383 | 29.915 | 1.00 | 32.78 | C |
| ATOM | 3125 | CE1 | TYR | L | 186 | 35.919 | 17.236 | 29.139 | 1.00 | 30.87 | C |
| ATOM | 3126 | CZ | TYR | L | 186 | 36.977 | 17.072 | 28.283 | 1.00 | 30.03 | C |
| ATOM | 3127 | OH | TYR | L | 186 | 37.040 | 15.931 | 27.529 | 1.00 | 29.71 | O |
| ATOM | 3128 | CE2 | TYR | L | 186 | 37.950 | 18.043 | 28.177 | 1.00 | 30.52 | C |
| ATOM | 3129 | CD2 | TYR | L | 186 | 37.860 | 19.190 | 28.966 | 1.00 | 33.34 | C |
| ATOM | 3130 | C | TYR | L | 186 | 37.923 | 19.494 | 32.596 | 1.00 | 41.22 | C |
| ATOM | 3131 | O | TYR | L | 186 | 37.752 | 18.361 | 33.019 | 1.00 | 41.67 | O |
| ATOM | 3132 | N | GLU | L | 187 | 39.131 | 20.042 | 32.473 | 1.00 | 42.26 | N |
| ATOM | 3133 | CA | GLU | L | 187 | 40.368 | 19.232 | 32.528 | 1.00 | 43.08 | C |
| ATOM | 3134 | CB | GLU | L | 187 | 41.604 | 20.055 | 32.115 | 1.00 | 42.99 | C |
| ATOM | 3135 | CG | GLU | L | 187 | 41.829 | 20.173 | 30.595 | 1.00 | 43.43 | C |
| ATOM | 3136 | CD | GLU | L | 187 | 42.245 | 18.855 | 29.907 | 1.00 | 44.55 | C |
| ATOM | 3137 | OE1 | GLU | L | 187 | 42.540 | 17.846 | 30.602 | 1.00 | 45.23 | O |
| ATOM | 3138 | OE2 | GLU | L | 187 | 42.278 | 18.838 | 28.654 | 1.00 | 43.06 | O |
| ATOM | 3139 | C | GLU | L | 187 | 40.635 | 18.532 | 33.860 | 1.00 | 43.47 | C |
| ATOM | 3140 | O | GLU | L | 187 | 41.349 | 17.515 | 33.893 | 1.00 | 43.73 | O |
| ATOM | 3141 | N | LYS | L | 188 | 40.063 | 19.073 | 34.940 | 1.00 | 43.56 | N |
| ATOM | 3142 | CA | LYS | L | 188 | 40.234 | 18.511 | 36.288 | 1.00 | 43.40 | C |
| ATOM | 3143 | CB | LYS | L | 188 | 40.260 | 19.619 | 37.350 | 1.00 | 43.55 | C |
| ATOM | 3144 | CG | LYS | L | 188 | 38.999 | 20.468 | 37.456 | 1.00 | 43.85 | C |
| ATOM | 3145 | CD | LYS | L | 188 | 39.233 | 21.597 | 38.451 | 1.00 | 44.32 | C |
| ATOM | 3146 | CE | LYS | L | 188 | 37.997 | 22.438 | 38.654 | 1.00 | 44.77 | C |
| ATOM | 3147 | NZ | LYS | L | 188 | 38.352 | 23.882 | 38.795 | 1.00 | 44.39 | N |
| ATOM | 3148 | C | LYS | L | 188 | 39.203 | 17.433 | 36.647 | 1.00 | 43.08 | C |
| ATOM | 3149 | O | LYS | L | 188 | 38.877 | 17.234 | 37.828 | 1.00 | 43.38 | O |
| ATOM | 3150 | N | HIS | L | 189 | 38.703 | 16.741 | 35.625 | 1.00 | 42.22 | N |
| ATOM | 3151 | CA | HIS | L | 189 | 37.845 | 15.578 | 35.818 | 1.00 | 41.29 | C |
| ATOM | 3152 | CB | HIS | L | 189 | 36.374 | 15.964 | 35.724 | 1.00 | 41.29 | C |
| ATOM | 3153 | CG | HIS | L | 189 | 35.905 | 16.848 | 36.836 | 1.00 | 41.23 | C |
| ATOM | 3154 | ND1 | HIS | L | 189 | 35.948 | 16.466 | 38.160 | 1.00 | 41.70 | N |
| ATOM | 3155 | CE1 | HIS | L | 189 | 35.457 | 17.437 | 38.912 | 1.00 | 42.93 | C |
| ATOM | 3156 | NE2 | HIS | L | 189 | 35.093 | 18.433 | 38.123 | 1.00 | 43.21 | N |
| ATOM | 3157 | CD2 | HIS | L | 189 | 35.362 | 18.089 | 36.819 | 1.00 | 42.20 | C |
| ATOM | 3158 | C | HIS | L | 189 | 38.184 | 14.525 | 34.778 | 1.00 | 40.71 | C |
| ATOM | 3159 | O | HIS | L | 189 | 38.800 | 14.833 | 33.761 | 1.00 | 40.77 | O |
| ATOM | 3160 | N | LYS | L | 190 | 37.771 | 13.288 | 35.029 | 1.00 | 39.80 | N |
| ATOM | 3161 | CA | LYS | L | 190 | 38.190 | 12.159 | 34.208 | 1.00 | 39.06 | C |
| ATOM | 3162 | CB | LYS | L | 190 | 38.926 | 11.141 | 35.086 | 1.00 | 39.23 | C |
| ATOM | 3163 | CG | LYS | L | 190 | 39.492 | 9.940 | 34.343 | 1.00 | 40.83 | C |
| ATOM | 3164 | CD | LYS | L | 190 | 40.485 | 9.147 | 35.208 | 1.00 | 43.87 | C |
| ATOM | 3165 | CE | LYS | L | 190 | 41.910 | 9.744 | 35.129 | 1.00 | 45.16 | C |
| ATOM | 3166 | NZ | LYS | L | 190 | 42.949 | 8.890 | 35.805 | 1.00 | 46.07 | N |
| ATOM | 3167 | C | LYS | L | 190 | 37.026 | 11.498 | 33.456 | 1.00 | 38.31 | C |
| ATOM | 3168 | O | LYS | L | 190 | 37.069 | 11.342 | 32.230 | 1.00 | 38.58 | O |
| ATOM | 3169 | N | VAL | L | 191 | 35.996 | 11.096 | 34.191 | 1.00 | 36.77 | N |
| ATOM | 3170 | CA | VAL | L | 191 | 34.871 | 10.425 | 33.581 | 1.00 | 35.46 | C |
| ATOM | 3171 | CB | VAL | L | 191 | 34.312 | 9.301 | 34.464 | 1.00 | 35.62 | C |
| ATOM | 3172 | CG1 | VAL | L | 191 | 33.433 | 8.379 | 33.638 | 1.00 | 35.63 | C |
| ATOM | 3173 | CG2 | VAL | L | 191 | 35.442 | 8.508 | 35.105 | 1.00 | 36.44 | C |
| ATOM | 3174 | C | VAL | L | 191 | 33.762 | 11.421 | 33.239 | 1.00 | 34.46 | C |
| ATOM | 3175 | O | VAL | L | 191 | 33.332 | 12.228 | 34.074 | 1.00 | 33.95 | O |
| ATOM | 3176 | N | TYR | L | 192 | 33.335 | 11.362 | 31.983 | 1.00 | 32.97 | N |
| ATOM | 3177 | CA | TYR | L | 192 | 32.212 | 12.131 | 31.507 | 1.00 | 31.40 | C |
| ATOM | 3178 | CB | TYR | L | 192 | 32.660 | 13.115 | 30.443 | 1.00 | 31.27 | C |
| ATOM | 3179 | CG | TYR | L | 192 | 33.426 | 14.263 | 31.046 | 1.00 | 30.00 | C |
| ATOM | 3180 | CD1 | TYR | L | 192 | 34.808 | 14.179 | 31.271 | 1.00 | 30.19 | C |
| ATOM | 3181 | CE1 | TYR | L | 192 | 35.507 | 15.240 | 31.847 | 1.00 | 29.43 | C |
| ATOM | 3182 | CZ | TYR | L | 192 | 34.810 | 16.384 | 32.207 | 1.00 | 30.04 | C |
| ATOM | 3183 | OH | TYR | L | 192 | 35.447 | 17.457 | 32.783 | 1.00 | 31.32 | O |
| ATOM | 3184 | CE2 | TYR | L | 192 | 33.447 | 16.475 | 31.993 | 1.00 | 30.09 | C |
| ATOM | 3185 | CD2 | TYR | L | 192 | 32.769 | 15.415 | 31.422 | 1.00 | 29.28 | C |
| ATOM | 3186 | C | TYR | L | 192 | 31.218 | 11.128 | 30.990 | 1.00 | 30.76 | C |
| ATOM | 3187 | O | TYR | L | 192 | 31.544 | 10.309 | 30.127 | 1.00 | 31.01 | O |
| ATOM | 3188 | N | ALA | L | 193 | 30.023 | 11.159 | 31.568 | 1.00 | 29.52 | N |
| ATOM | 3189 | CA | ALA | L | 193 | 29.047 | 10.111 | 31.343 | 1.00 | 28.62 | C |
| ATOM | 3190 | CB | ALA | L | 193 | 29.016 | 9.189 | 32.531 | 1.00 | 28.45 | C |
| ATOM | 3191 | C | ALA | L | 193 | 27.655 | 10.673 | 31.065 | 1.00 | 28.30 | C |
| ATOM | 3192 | O | ALA | L | 193 | 27.207 | 11.629 | 31.707 | 1.00 | 27.85 | O |
| ATOM | 3193 | N | CYS | L | 194 | 26.980 | 10.068 | 30.095 | 1.00 | 27.69 | N |
| ATOM | 3194 | CA | CYS | L | 194 | 25.624 | 10.453 | 29.737 | 1.00 | 27.19 | C |
| ATOM | 3195 | CB | CYS | L | 194 | 25.566 | 10.876 | 28.255 | 1.00 | 26.91 | C |
| ATOM | 3196 | SG | CYS | L | 194 | 23.875 | 11.065 | 27.677 | 1.00 | 30.30 | S |
| ATOM | 3197 | C | CYS | L | 194 | 24.695 | 9.264 | 30.039 | 1.00 | 26.40 | C |
| ATOM | 3198 | O | CYS | L | 194 | 24.816 | 8.197 | 29.417 | 1.00 | 26.49 | O |
| ATOM | 3199 | N | GLU | L | 195 | 23.793 | 9.440 | 31.005 | 1.00 | 25.29 | N |
| ATOM | 3200 | CA | GLU | L | 195 | 22.898 | 8.360 | 31.429 | 1.00 | 24.43 | C |
| ATOM | 3201 | CB | GLU | L | 195 | 22.954 | 8.134 | 32.946 | 1.00 | 24.46 | C |
| ATOM | 3202 | CG | GLU | L | 195 | 22.290 | 6.823 | 33.381 | 1.00 | 26.74 | C |
| ATOM | 3203 | CD | GLU | L | 195 | 21.766 | 6.828 | 34.825 | 1.00 | 29.63 | C |
| ATOM | 3204 | OE1 | GLU | L | 195 | 21.099 | 7.812 | 35.229 | 1.00 | 30.77 | O |
| ATOM | 3205 | OE2 | GLU | L | 195 | 21.995 | 5.821 | 35.545 | 1.00 | 30.38 | O |
| ATOM | 3206 | C | GLU | L | 195 | 21.463 | 8.609 | 30.975 | 1.00 | 23.41 | C |
| ATOM | 3207 | O | GLU | L | 195 | 20.853 | 9.617 | 31.339 | 1.00 | 22.75 | O |
| ATOM | 3208 | N | VAL | L | 196 | 20.936 | 7.662 | 30.203 | 1.00 | 22.44 | N |
| ATOM | 3209 | CA | VAL | L | 196 | 19.653 | 7.817 | 29.532 | 1.00 | 22.05 | C |
| ATOM | 3210 | CB | VAL | L | 196 | 19.787 | 7.626 | 27.986 | 1.00 | 21.87 | C |
| ATOM | 3211 | CG1 | VAL | L | 196 | 18.432 | 7.478 | 27.323 | 1.00 | 21.13 | C |
| ATOM | 3212 | CG2 | VAL | L | 196 | 20.540 | 8.788 | 27.372 | 1.00 | 21.35 | C |
| ATOM | 3213 | C | VAL | L | 196 | 18.600 | 6.873 | 30.082 | 1.00 | 22.10 | C |
| ATOM | 3214 | O | VAL | L | 196 | 18.811 | 5.668 | 30.139 | 1.00 | 22.05 | O |
| ATOM | 3215 | N | THR | L | 197 | 17.471 | 7.446 | 30.487 | 1.00 | 22.25 | N |
| ATOM | 3216 | CA | THR | L | 197 | 16.314 | 6.696 | 30.931 | 1.00 | 22.68 | C |
| ATOM | 3217 | CB | THR | L | 197 | 15.869 | 7.138 | 32.346 | 1.00 | 22.77 | C |
| ATOM | 3218 | OG1 | THR | L | 197 | 16.950 | 6.949 | 33.268 | 1.00 | 22.12 | O |
| ATOM | 3219 | CG2 | THR | L | 197 | 14.646 | 6.331 | 32.818 | 1.00 | 22.32 | C |
| ATOM | 3220 | C | THR | L | 197 | 15.184 | 6.929 | 29.934 | 1.00 | 23.18 | C |
| ATOM | 3221 | O | THR | L | 197 | 14.953 | 8.065 | 29.500 | 1.00 | 23.56 | O |
| ATOM | 3222 | N | HIS | L | 198 | 14.483 | 5.856 | 29.577 | 1.00 | 23.47 | N |
| ATOM | 3223 | CA | HIS | L | 198 | 13.455 | 5.893 | 28.536 | 1.00 | 23.73 | C |
| ATOM | 3224 | CB | HIS | L | 198 | 14.087 | 6.138 | 27.159 | 1.00 | 23.20 | C |
| ATOM | 3225 | CG | HIS | L | 198 | 13.094 | 6.297 | 26.052 | 1.00 | 21.81 | C |
| ATOM | 3226 | ND1 | HIS | L | 198 | 12.602 | 5.227 | 25.335 | 1.00 | 20.89 | N |
| ATOM | 3227 | CE1 | HIS | L | 198 | 11.749 | 5.666 | 24.426 | 1.00 | 20.54 | C |
| ATOM | 3228 | NE2 | HIS | L | 198 | 11.664 | 6.981 | 24.534 | 1.00 | 19.43 | N |
| ATOM | 3229 | CD2 | HIS | L | 198 | 12.501 | 7.401 | 25.538 | 1.00 | 19.79 | C |
| ATOM | 3230 | C | HIS | L | 198 | 12.680 | 4.583 | 28.534 | 1.00 | 24.67 | C |
| ATOM | 3231 | O | HIS | L | 198 | 13.236 | 3.527 | 28.843 | 1.00 | 24.96 | O |
| ATOM | 3232 | N | GLN | L | 199 | 11.403 | 4.667 | 28.161 | 1.00 | 25.69 | N |
| ATOM | 3233 | CA | GLN | L | 199 | 10.468 | 3.537 | 28.194 | 1.00 | 26.64 | C |
| ATOM | 3234 | CB | GLN | L | 199 | 9.087 | 3.985 | 27.698 | 1.00 | 26.66 | C |
| ATOM | 3235 | CG | GLN | L | 199 | 7.947 | 3.152 | 28.246 | 1.00 | 28.30 | C |
| ATOM | 3236 | CD | GLN | L | 199 | 6.658 | 3.287 | 27.447 | 1.00 | 30.01 | C |
| ATOM | 3237 | OE1 | GLN | L | 199 | 6.331 | 4.359 | 26.943 | 1.00 | 31.66 | O |
| ATOM | 3238 | NE2 | GLN | L | 199 | 5.918 | 2.188 | 27.332 | 1.00 | 29.23 | N |
| ATOM | 3239 | C | GLN | L | 199 | 10.920 | 2.267 | 27.434 | 1.00 | 26.80 | C |
| ATOM | 3240 | O | GLN | L | 199 | 10.518 | 1.166 | 27.809 | 1.00 | 27.13 | O |
| ATOM | 3241 | N | GLY | L | 200 | 11.734 | 2.426 | 26.386 | 1.00 | 26.76 | N |
| ATOM | 3242 | CA | GLY | L | 200 | 12.224 | 1.309 | 25.570 | 1.00 | 27.10 | C |
| ATOM | 3243 | C | GLY | L | 200 | 13.507 | 0.654 | 26.068 | 1.00 | 27.75 | C |
| ATOM | 3244 | O | GLY | L | 200 | 14.015 | −0.305 | 25.466 | 1.00 | 27.39 | O |
| ATOM | 3245 | N | LEU | L | 201 | 14.024 | 1.160 | 27.182 | 1.00 | 28.25 | N |
| ATOM | 3246 | CA | LEU | L | 201 | 15.190 | 0.571 | 27.823 | 1.00 | 28.77 | C |
| ATOM | 3247 | CB | LEU | L | 201 | 16.198 | 1.671 | 28.160 | 1.00 | 28.51 | C |
| ATOM | 3248 | CG | LEU | L | 201 | 16.739 | 2.470 | 26.967 | 1.00 | 28.48 | C |
| ATOM | 3249 | CD1 | LEU | L | 201 | 17.667 | 3.593 | 27.420 | 1.00 | 27.14 | C |
| ATOM | 3250 | CD2 | LEU | L | 201 | 17.446 | 1.554 | 25.968 | 1.00 | 27.47 | C |
| ATOM | 3251 | C | LEU | L | 201 | 14.801 | −0.215 | 29.083 | 1.00 | 29.34 | C |
| ATOM | 3252 | O | LEU | L | 201 | 14.187 | 0.350 | 29.995 | 1.00 | 29.24 | O |
| ATOM | 3253 | N | SER | L | 202 | 15.156 | −1.510 | 29.143 | 1.00 | 30.00 | N |
| ATOM | 3254 | CA | SER | L | 202 | 14.904 | −2.313 | 30.371 | 1.00 | 30.19 | C |
| ATOM | 3255 | CB | SER | L | 202 | 15.501 | −3.736 | 30.329 | 1.00 | 30.25 | C |
| ATOM | 3256 | OG | SER | L | 202 | 15.918 | −4.156 | 29.037 | 1.00 | 32.81 | O |
| ATOM | 3257 | C | SER | L | 202 | 15.503 | −1.567 | 31.562 | 1.00 | 29.81 | C |
| ATOM | 3258 | O | SER | L | 202 | 14.864 | −1.439 | 32.613 | 1.00 | 29.08 | O |
| ATOM | 3259 | N | SER | L | 203 | 16.735 | −1.087 | 31.375 | 1.00 | 29.79 | N |
| ATOM | 3260 | CA | SER | L | 203 | 17.429 | −0.300 | 32.390 | 1.00 | 29.98 | C |
| ATOM | 3261 | CB | SER | L | 203 | 18.279 | −1.204 | 33.303 | 1.00 | 30.19 | C |
| ATOM | 3262 | OG | SER | L | 203 | 19.558 | −1.482 | 32.755 | 1.00 | 30.29 | O |
| ATOM | 3263 | C | SER | L | 203 | 18.268 | 0.822 | 31.766 | 1.00 | 30.15 | C |
| ATOM | 3264 | O | SER | L | 203 | 18.686 | 0.722 | 30.609 | 1.00 | 29.89 | O |
| ATOM | 3265 | N | PRO | L | 204 | 18.510 | 1.897 | 32.539 | 1.00 | 30.53 | N |
| ATOM | 3266 | CA | PRO | L | 204 | 19.263 | 3.060 | 32.101 | 1.00 | 30.88 | C |
| ATOM | 3267 | CB | PRO | L | 204 | 19.568 | 3.775 | 33.414 | 1.00 | 30.85 | C |
| ATOM | 3268 | CG | PRO | L | 204 | 18.394 | 3.477 | 34.248 | 1.00 | 30.76 | C |
| ATOM | 3269 | CD | PRO | L | 204 | 18.066 | 2.053 | 33.938 | 1.00 | 30.50 | C |
| ATOM | 3270 | C | PRO | L | 204 | 20.558 | 2.692 | 31.415 | 1.00 | 31.25 | C |
| ATOM | 3271 | O | PRO | L | 204 | 21.291 | 1.842 | 31.910 | 1.00 | 31.67 | O |
| ATOM | 3272 | N | VAL | L | 205 | 20.818 | 3.337 | 30.279 | 1.00 | 31.51 | N |
| ATOM | 3273 | CA | VAL | L | 205 | 22.020 | 3.120 | 29.486 | 1.00 | 31.40 | C |
| ATOM | 3274 | CB | VAL | L | 205 | 21.657 | 2.951 | 27.979 | 1.00 | 31.41 | C |
| ATOM | 3275 | CG1 | VAL | L | 205 | 22.845 | 3.246 | 27.065 | 1.00 | 31.10 | C |
| ATOM | 3276 | CG2 | VAL | L | 205 | 21.112 | 1.565 | 27.711 | 1.00 | 31.32 | C |
| ATOM | 3277 | C | VAL | L | 205 | 22.951 | 4.317 | 29.687 | 1.00 | 31.63 | C |
| ATOM | 3278 | O | VAL | L | 205 | 22.511 | 5.466 | 29.626 | 1.00 | 31.51 | O |
| ATOM | 3279 | N | THR | L | 206 | 24.231 | 4.033 | 29.936 | 1.00 | 31.86 | N |
| ATOM | 3280 | CA | THR | L | 206 | 25.258 | 5.060 | 30.103 | 1.00 | 31.97 | C |
| ATOM | 3281 | CB | THR | L | 206 | 26.078 | 4.821 | 31.394 | 1.00 | 31.96 | C |
| ATOM | 3282 | OG1 | THR | L | 206 | 25.209 | 4.441 | 32.466 | 1.00 | 31.47 | O |
| ATOM | 3283 | CG2 | THR | L | 206 | 26.852 | 6.068 | 31.802 | 1.00 | 32.52 | C |
| ATOM | 3284 | C | THR | L | 206 | 26.216 | 4.991 | 28.921 | 1.00 | 32.25 | C |
| ATOM | 3285 | O | THR | L | 206 | 26.523 | 3.898 | 28.442 | 1.00 | 32.41 | O |
| ATOM | 3286 | N | LYS | L | 207 | 26.664 | 6.150 | 28.440 | 1.00 | 32.45 | N |
| ATOM | 3287 | CA | LYS | L | 207 | 27.764 | 6.222 | 27.482 | 1.00 | 32.86 | C |
| ATOM | 3288 | CB | LYS | L | 207 | 27.313 | 6.743 | 26.107 | 1.00 | 32.75 | C |
| ATOM | 3289 | CG | LYS | L | 207 | 26.342 | 5.845 | 25.346 | 1.00 | 32.16 | C |
| ATOM | 3290 | CD | LYS | L | 207 | 26.992 | 4.593 | 24.784 | 1.00 | 31.00 | C |
| ATOM | 3291 | CE | LYS | L | 207 | 25.926 | 3.523 | 24.562 | 1.00 | 31.62 | C |
| ATOM | 3292 | NZ | LYS | L | 207 | 26.407 | 2.365 | 23.767 | 1.00 | 32.26 | N |
| ATOM | 3293 | C | LYS | L | 207 | 28.842 | 7.126 | 28.055 | 1.00 | 33.53 | C |
| ATOM | 3294 | O | LYS | L | 207 | 28.586 | 8.302 | 28.371 | 1.00 | 33.63 | O |
| ATOM | 3295 | N | SER | L | 208 | 30.048 | 6.574 | 28.186 | 1.00 | 34.16 | N |
| ATOM | 3296 | CA | SER | L | 208 | 31.143 | 7.266 | 28.860 | 1.00 | 35.01 | C |
| ATOM | 3297 | CB | SER | L | 208 | 31.528 | 6.535 | 30.149 | 1.00 | 35.00 | C |
| ATOM | 3298 | OG | SER | L | 208 | 30.389 | 6.024 | 30.813 | 1.00 | 35.82 | O |
| ATOM | 3299 | C | SER | L | 208 | 32.379 | 7.384 | 27.986 | 1.00 | 35.49 | C |
| ATOM | 3300 | O | SER | L | 208 | 32.573 | 6.621 | 27.042 | 1.00 | 35.21 | O |
| ATOM | 3301 | N | PHE | L | 209 | 33.198 | 8.373 | 28.303 | 1.00 | 36.16 | N |
| ATOM | 3302 | CA | PHE | L | 209 | 34.569 | 8.377 | 27.865 | 1.00 | 37.25 | C |
| ATOM | 3303 | CB | PHE | L | 209 | 34.776 | 9.229 | 26.605 | 1.00 | 36.88 | C |
| ATOM | 3304 | CG | PHE | L | 209 | 34.620 | 10.707 | 26.827 | 1.00 | 35.64 | C |
| ATOM | 3305 | CD1 | PHE | L | 209 | 35.723 | 11.497 | 27.148 | 1.00 | 34.28 | C |
| ATOM | 3306 | CE1 | PHE | L | 209 | 35.580 | 12.857 | 27.359 | 1.00 | 33.43 | C |
| ATOM | 3307 | CZ | PHE | L | 209 | 34.328 | 13.445 | 27.234 | 1.00 | 33.20 | C |
| ATOM | 3308 | CE2 | PHE | L | 209 | 33.231 | 12.672 | 26.902 | 1.00 | 32.91 | C |
| ATOM | 3309 | CD2 | PHE | L | 209 | 33.376 | 11.314 | 26.697 | 1.00 | 33.37 | C |
| ATOM | 3310 | C | PHE | L | 209 | 35.423 | 8.857 | 29.026 | 1.00 | 38.51 | C |
| ATOM | 3311 | O | PHE | L | 209 | 34.953 | 9.596 | 29.896 | 1.00 | 38.45 | O |
| ATOM | 3312 | N | ASN | L | 210 | 36.665 | 8.389 | 29.060 | 1.00 | 40.26 | N |
| ATOM | 3313 | CA | ASN | L | 210 | 37.640 | 8.904 | 29.996 | 1.00 | 41.96 | C |
| ATOM | 3314 | CB | ASN | L | 210 | 38.549 | 7.785 | 30.510 | 1.00 | 42.13 | C |
| ATOM | 3315 | CG | ASN | L | 210 | 37.768 | 6.585 | 31.077 | 1.00 | 43.00 | C |
| ATOM | 3316 | OD1 | ASN | L | 210 | 36.546 | 6.640 | 31.289 | 1.00 | 43.41 | O |
| ATOM | 3317 | ND2 | ASN | L | 210 | 38.486 | 5.493 | 31.324 | 1.00 | 43.82 | N |
| ATOM | 3318 | C | ASN | L | 210 | 38.423 | 9.958 | 29.242 | 1.00 | 43.10 | C |
| ATOM | 3319 | O | ASN | L | 210 | 39.078 | 9.651 | 28.241 | 1.00 | 43.57 | O |
| ATOM | 3320 | N | ARG | L | 211 | 38.320 | 11.200 | 29.698 | 1.00 | 44.43 | N |
| ATOM | 3321 | CA | ARG | L | 211 | 38.902 | 12.336 | 29.000 | 1.00 | 46.25 | C |
| ATOM | 3322 | CB | ARG | L | 211 | 39.027 | 13.534 | 29.953 | 1.00 | 46.55 | C |
| ATOM | 3323 | CG | ARG | L | 211 | 39.703 | 14.767 | 29.337 | 1.00 | 47.95 | C |
| ATOM | 3324 | CD | ARG | L | 211 | 39.824 | 15.967 | 30.298 | 1.00 | 49.97 | C |
| ATOM | 3325 | NE | ARG | L | 211 | 40.319 | 15.650 | 31.646 | 1.00 | 52.52 | N |
| ATOM | 3326 | CZ | ARG | L | 211 | 41.444 | 14.987 | 31.930 | 1.00 | 53.52 | C |
| ATOM | 3327 | NH1 | ARG | L | 211 | 42.222 | 14.507 | 30.966 | 1.00 | 54.09 | N |
| ATOM | 3328 | NH2 | ARG | L | 211 | 41.777 | 14.775 | 33.196 | 1.00 | 54.01 | N |
| ATOM | 3329 | C | ARG | L | 211 | 40.249 | 12.007 | 28.334 | 1.00 | 47.34 | C |
| ATOM | 3330 | O | ARG | L | 211 | 41.151 | 11.446 | 28.965 | 1.00 | 47.43 | O |
| ATOM | 3331 | N | GLY | L | 212 | 40.359 | 12.344 | 27.050 | 1.00 | 48.61 | N |
| ATOM | 3332 | CA | GLY | L | 212 | 41.604 | 12.199 | 26.302 | 1.00 | 49.94 | C |
| ATOM | 3333 | C | GLY | L | 212 | 41.807 | 10.859 | 25.615 | 1.00 | 51.11 | C |
| ATOM | 3334 | O | GLY | L | 212 | 42.732 | 10.117 | 25.974 | 1.00 | 51.30 | O |
| ATOM | 3335 | N | GLU | L | 213 | 40.943 | 10.550 | 24.638 | 1.00 | 51.80 | N |
| ATOM | 3336 | CA | GLU | L | 213 | 41.137 | 9.409 | 23.719 | 1.00 | 52.60 | C |
| ATOM | 3337 | CB | GLU | L | 213 | 40.546 | 8.106 | 24.276 | 1.00 | 52.92 | C |
| ATOM | 3338 | CG | GLU | L | 213 | 41.530 | 7.249 | 25.084 | 1.00 | 54.07 | C |
| ATOM | 3339 | CD | GLU | L | 213 | 41.124 | 7.095 | 26.547 | 1.00 | 55.51 | C |
| ATOM | 3340 | OE1 | GLU | L | 213 | 40.379 | 6.133 | 26.857 | 1.00 | 55.69 | O |
| ATOM | 3341 | OE2 | GLU | L | 213 | 41.566 | 7.915 | 27.385 | 1.00 | 55.33 | O |
| ATOM | 3342 | C | GLU | L | 213 | 40.571 | 9.677 | 22.316 | 1.00 | 52.71 | C |
| ATOM | 3343 | O | GLU | L | 213 | 41.009 | 9.078 | 21.327 | 1.00 | 52.59 | O |
| ATOM | 3344 | N | GLU | I | 1 | 12.458 | 35.825 | 3.643 | 1.00 | 41.80 | N |
| ATOM | 3345 | CA | GLU | I | 1 | 13.209 | 36.044 | 2.371 | 1.00 | 41.71 | C |
| ATOM | 3346 | CB | GLU | I | 1 | 13.550 | 34.691 | 1.707 | 1.00 | 42.19 | C |
| ATOM | 3347 | CG | GLU | I | 1 | 14.064 | 33.573 | 2.667 | 1.00 | 44.36 | C |
| ATOM | 3348 | CD | GLU | I | 1 | 15.479 | 33.822 | 3.246 | 1.00 | 47.21 | C |
| ATOM | 3349 | OE1 | GLU | I | 1 | 16.404 | 34.212 | 2.488 | 1.00 | 47.49 | O |
| ATOM | 3350 | OE2 | GLU | I | 1 | 15.664 | 33.600 | 4.468 | 1.00 | 47.66 | O |
| ATOM | 3351 | C | GLU | I | 1 | 12.401 | 36.959 | 1.429 | 1.00 | 40.73 | C |
| ATOM | 3352 | O | GLU | I | 1 | 11.596 | 37.791 | 1.890 | 1.00 | 41.25 | O |
| ATOM | 3353 | N | VAL | I | 2 | 12.611 | 36.812 | 0.124 | 1.00 | 38.84 | N |
| ATOM | 3354 | CA | VAL | I | 2 | 11.920 | 37.634 | −0.868 | 1.00 | 37.10 | C |
| ATOM | 3355 | CB | VAL | I | 2 | 12.823 | 37.907 | −2.105 | 1.00 | 37.29 | C |
| ATOM | 3356 | CG1 | VAL | I | 2 | 12.064 | 38.669 | −3.180 | 1.00 | 36.67 | C |
| ATOM | 3357 | CG2 | VAL | I | 2 | 14.083 | 38.671 | −1.682 | 1.00 | 37.25 | C |
| ATOM | 3358 | C | VAL | I | 2 | 10.583 | 37.022 | −1.310 | 1.00 | 35.77 | C |
| ATOM | 3359 | O | VAL | I | 2 | 10.466 | 35.803 | −1.481 | 1.00 | 36.18 | O |
| ATOM | 3360 | N | GLN | I | 3 | 9.580 | 37.876 | −1.483 | 1.00 | 33.39 | N |
| ATOM | 3361 | CA | GLN | I | 3 | 8.298 | 37.455 | −2.012 | 1.00 | 31.18 | C |
| ATOM | 3362 | CB | GLN | I | 3 | 7.188 | 37.707 | −0.996 | 1.00 | 31.56 | C |
| ATOM | 3363 | CG | GLN | I | 3 | 7.149 | 36.732 | 0.156 | 1.00 | 33.33 | C |
| ATOM | 3364 | CD | GLN | I | 3 | 5.749 | 36.547 | 0.688 | 1.00 | 35.43 | C |
| ATOM | 3365 | OE1 | GLN | I | 3 | 5.469 | 36.852 | 1.848 | 1.00 | 35.70 | O |
| ATOM | 3366 | NE2 | GLN | I | 3 | 4.846 | 36.062 | −0.169 | 1.00 | 36.18 | N |
| ATOM | 3367 | C | GLN | I | 3 | 7.983 | 38.226 | −3.281 | 1.00 | 29.18 | C |
| ATOM | 3368 | O | GLN | I | 3 | 8.186 | 39.438 | −3.350 | 1.00 | 29.00 | O |
| ATOM | 3369 | N | LEU | I | 4 | 7.481 | 37.521 | −4.283 | 1.00 | 26.58 | N |
| ATOM | 3370 | CA | LEU | I | 4 | 6.942 | 38.175 | −5.460 | 1.00 | 24.34 | C |
| ATOM | 3371 | CB | LEU | I | 4 | 7.484 | 37.537 | −6.740 | 1.00 | 23.70 | C |
| ATOM | 3372 | CG | LEU | I | 4 | 9.000 | 37.411 | −6.924 | 1.00 | 23.51 | C |
| ATOM | 3373 | CD1 | LEU | I | 4 | 9.291 | 36.748 | −8.261 | 1.00 | 23.12 | C |
| ATOM | 3374 | CD2 | LEU | I | 4 | 9.744 | 38.744 | −6.826 | 1.00 | 23.01 | C |
| ATOM | 3375 | C | LEU | I | 4 | 5.420 | 38.117 | −5.409 | 1.00 | 23.17 | C |
| ATOM | 3376 | O | LEU | I | 4 | 4.835 | 37.052 | −5.205 | 1.00 | 22.95 | O |
| ATOM | 3377 | N | VAL | I | 5 | 4.784 | 39.266 | −5.578 | 1.00 | 21.84 | N |
| ATOM | 3378 | CA | VAL | I | 5 | 3.335 | 39.357 | −5.484 | 1.00 | 21.44 | C |
| ATOM | 3379 | CB | VAL | I | 5 | 2.893 | 40.220 | −4.264 | 1.00 | 21.66 | C |
| ATOM | 3380 | CG1 | VAL | I | 5 | 1.407 | 40.549 | −4.321 | 1.00 | 21.58 | C |
| ATOM | 3381 | CG2 | VAL | I | 5 | 3.229 | 39.498 | −2.949 | 1.00 | 21.29 | C |
| ATOM | 3382 | C | VAL | I | 5 | 2.760 | 39.903 | −6.792 | 1.00 | 21.21 | C |
| ATOM | 3383 | O | VAL | I | 5 | 3.088 | 41.021 | −7.212 | 1.00 | 21.07 | O |
| ATOM | 3384 | N | GLN | I | 6 | 1.904 | 39.104 | −7.428 | 1.00 | 20.18 | N |
| ATOM | 3385 | CA | GLN | I | 6 | 1.390 | 39.432 | −8.751 | 1.00 | 19.53 | C |
| ATOM | 3386 | CB | GLN | I | 6 | 1.431 | 38.200 | −9.657 | 1.00 | 19.13 | C |
| ATOM | 3387 | CG | GLN | I | 6 | 2.852 | 37.837 | −10.039 | 1.00 | 18.66 | C |
| ATOM | 3388 | CD | GLN | I | 6 | 2.952 | 36.726 | −11.058 | 1.00 | 20.03 | C |
| ATOM | 3389 | OE1 | GLN | I | 6 | 3.497 | 35.655 | −10.774 | 1.00 | 20.75 | O |
| ATOM | 3390 | NE2 | GLN | I | 6 | 2.449 | 36.975 | −12.259 | 1.00 | 19.52 | N |
| ATOM | 3391 | C | GLN | I | 6 | 0.008 | 40.055 | −8.683 | 1.00 | 19.34 | C |
| ATOM | 3392 | O | GLN | I | 6 | −0.736 | 39.800 | −7.747 | 1.00 | 19.29 | O |
| ATOM | 3393 | N | SER | I | 7 | −0.333 | 40.893 | −9.655 | 1.00 | 19.40 | N |
| ATOM | 3394 | CA | SER | I | 7 | −1.686 | 41.466 | −9.692 | 1.00 | 19.87 | C |
| ATOM | 3395 | CB | SER | I | 7 | −1.757 | 42.629 | −10.679 | 1.00 | 19.77 | C |
| ATOM | 3396 | OG | SER | I | 7 | −0.956 | 42.374 | −11.813 | 1.00 | 21.08 | O |
| ATOM | 3397 | C | SER | I | 7 | −2.779 | 40.416 | −9.969 | 1.00 | 19.91 | C |
| ATOM | 3398 | O | SER | I | 7 | −2.480 | 39.271 | −10.306 | 1.00 | 19.81 | O |
| ATOM | 3399 | N | GLY | I | 8 | −4.042 | 40.811 | −9.825 | 1.00 | 20.27 | N |
| ATOM | 3400 | CA | GLY | I | 8 | −5.168 | 39.874 | −9.897 | 1.00 | 20.72 | C |
| ATOM | 3401 | C | GLY | I | 8 | −5.552 | 39.397 | −11.279 | 1.00 | 21.36 | C |
| ATOM | 3402 | O | GLY | I | 8 | −5.039 | 39.881 | −12.283 | 1.00 | 21.43 | O |
| ATOM | 3403 | N | ALA | I | 9 | −6.469 | 38.440 | −11.322 | 1.00 | 22.16 | N |
| ATOM | 3404 | CA | ALA | I | 9 | −6.927 | 37.850 | −12.578 | 1.00 | 22.82 | C |
| ATOM | 3405 | CB | ALA | I | 9 | −8.016 | 36.817 | −12.308 | 1.00 | 22.80 | C |
| ATOM | 3406 | C | ALA | I | 9 | −7.417 | 38.892 | −13.577 | 1.00 | 23.43 | C |
| ATOM | 3407 | O | ALA | I | 9 | −8.108 | 39.854 | −13.223 | 1.00 | 23.53 | O |
| ATOM | 3408 | N | GLU | I | 10 | −7.041 | 38.695 | −14.832 | 1.00 | 24.02 | N |
| ATOM | 3409 | CA | GLU | I | 10 | −7.427 | 39.606 | −15.901 | 1.00 | 24.42 | C |
| ATOM | 3410 | CB | GLU | I | 10 | −6.181 | 40.106 | −16.641 | 1.00 | 24.54 | C |
| ATOM | 3411 | CG | GLU | I | 10 | −5.281 | 41.057 | −15.834 | 1.00 | 26.02 | C |
| ATOM | 3412 | CD | GLU | I | 10 | −5.873 | 42.456 | −15.611 | 1.00 | 28.31 | C |
| ATOM | 3413 | OE1 | GLU | I | 10 | −6.968 | 42.778 | −16.140 | 1.00 | 29.54 | O |
| ATOM | 3414 | OE2 | GLU | I | 10 | −5.231 | 43.251 | −14.897 | 1.00 | 28.96 | O |
| ATOM | 3415 | C | GLU | I | 10 | −8.378 | 38.918 | −16.881 | 1.00 | 24.29 | C |
| ATOM | 3416 | O | GLU | I | 10 | −8.172 | 37.746 | −17.228 | 1.00 | 24.11 | O |
| ATOM | 3417 | N | VAL | I | 11 | −9.419 | 39.645 | −17.306 | 1.00 | 24.10 | N |
| ATOM | 3418 | CA | VAL | I | 11 | −10.351 | 39.174 | −18.341 | 1.00 | 23.63 | C |
| ATOM | 3419 | CB | VAL | I | 11 | −11.723 | 38.771 | −17.740 | 1.00 | 23.91 | C |
| ATOM | 3420 | CG1 | VAL | I | 11 | −12.714 | 38.387 | −18.842 | 1.00 | 22.85 | C |
| ATOM | 3421 | CG2 | VAL | I | 11 | −11.566 | 37.606 | −16.710 | 1.00 | 22.90 | C |
| ATOM | 3422 | C | VAL | I | 11 | −10.510 | 40.280 | −19.384 | 1.00 | 23.93 | C |
| ATOM | 3423 | O | VAL | I | 11 | −10.881 | 41.398 | −19.058 | 1.00 | 23.78 | O |
| ATOM | 3424 | N | LYS | I | 12 | −10.212 | 39.971 | −20.641 | 1.00 | 24.28 | N |
| ATOM | 3425 | CA | LYS | I | 12 | −10.079 | 41.006 | −21.665 | 1.00 | 24.69 | C |
| ATOM | 3426 | CB | LYS | I | 12 | −8.598 | 41.386 | −21.816 | 1.00 | 24.81 | C |
| ATOM | 3427 | CG | LYS | I | 12 | −7.998 | 42.136 | −20.624 | 1.00 | 24.78 | C |
| ATOM | 3428 | CD | LYS | I | 12 | −8.097 | 43.640 | −20.828 | 1.00 | 24.78 | C |
| ATOM | 3429 | CE | LYS | I | 12 | −8.097 | 44.380 | −19.518 | 1.00 | 25.05 | C |
| ATOM | 3430 | NZ | LYS | I | 12 | −7.669 | 45.782 | −19.723 | 1.00 | 26.07 | N |
| ATOM | 3431 | C | LYS | I | 12 | −10.645 | 40.598 | −23.026 | 1.00 | 24.95 | C |
| ATOM | 3432 | O | LYS | I | 12 | −10.780 | 39.405 | −23.314 | 1.00 | 24.93 | O |
| ATOM | 3433 | N | LYS | I | 13 | −10.971 | 41.599 | −23.845 | 1.00 | 25.13 | N |
| ATOM | 3434 | CA | LYS | I | 13 | −11.402 | 41.397 | −25.226 | 1.00 | 25.90 | C |
| ATOM | 3435 | CB | LYS | I | 13 | −12.341 | 42.529 | −25.697 | 1.00 | 26.09 | C |
| ATOM | 3436 | CG | LYS | I | 13 | −13.597 | 42.759 | −24.872 | 1.00 | 26.56 | C |
| ATOM | 3437 | CD | LYS | I | 13 | −14.582 | 41.638 | −25.032 | 1.00 | 28.10 | C |
| ATOM | 3438 | CE | LYS | I | 13 | −15.515 | 41.565 | −23.825 | 1.00 | 28.93 | C |
| ATOM | 3439 | NZ | LYS | I | 13 | −16.765 | 42.329 | −24.006 | 1.00 | 29.10 | N |
| ATOM | 3440 | C | LYS | I | 13 | −10.174 | 41.397 | −26.131 | 1.00 | 26.18 | C |
| ATOM | 3441 | O | LYS | I | 13 | −9.195 | 42.077 | −25.835 | 1.00 | 26.51 | O |
| ATOM | 3442 | N | PRO | I | 14 | −10.222 | 40.650 | −27.252 | 1.00 | 26.38 | N |
| ATOM | 3443 | CA | PRO | I | 14 | −9.085 | 40.692 | −28.165 | 1.00 | 26.74 | C |
| ATOM | 3444 | CB | PRO | I | 14 | −9.489 | 39.728 | −29.288 | 1.00 | 26.51 | C |
| ATOM | 3445 | CG | PRO | I | 14 | −10.970 | 39.597 | −29.169 | 1.00 | 26.86 | C |
| ATOM | 3446 | CD | PRO | I | 14 | −11.261 | 39.712 | −27.709 | 1.00 | 26.15 | C |
| ATOM | 3447 | C | PRO | I | 14 | −8.877 | 42.093 | −28.715 | 1.00 | 27.28 | C |
| ATOM | 3448 | O | PRO | I | 14 | −9.848 | 42.814 | −28.987 | 1.00 | 27.82 | O |
| ATOM | 3449 | N | GLY | I | 15 | −7.615 | 42.476 | −28.868 | 1.00 | 27.73 | N |
| ATOM | 3450 | CA | GLY | I | 15 | −7.260 | 43.824 | −29.306 | 1.00 | 27.79 | C |
| ATOM | 3451 | C | GLY | I | 15 | −7.076 | 44.805 | −28.162 | 1.00 | 27.97 | C |
| ATOM | 3452 | O | GLY | I | 15 | −6.593 | 45.924 | −28.362 | 1.00 | 27.83 | O |
| ATOM | 3453 | N | GLU | I | 16 | −7.465 | 44.407 | −26.954 | 1.00 | 28.16 | N |
| ATOM | 3454 | CA | GLU | I | 16 | −7.221 | 45.270 | −25.809 | 1.00 | 28.36 | C |
| ATOM | 3455 | CB | GLU | I | 16 | −8.192 | 44.983 | −24.663 | 1.00 | 28.45 | C |
| ATOM | 3456 | CG | GLU | I | 16 | −9.620 | 45.416 | −24.929 | 1.00 | 29.30 | C |
| ATOM | 3457 | CD | GLU | I | 16 | −10.456 | 45.491 | −23.662 | 1.00 | 31.14 | C |
| ATOM | 3458 | OE1 | GLU | I | 16 | −10.878 | 46.622 | −23.321 | 1.00 | 32.99 | O |
| ATOM | 3459 | OE2 | GLU | I | 16 | −10.685 | 44.440 | −23.004 | 1.00 | 29.91 | O |
| ATOM | 3460 | C | GLU | I | 16 | −5.761 | 45.194 | −25.343 | 1.00 | 28.28 | C |
| ATOM | 3461 | O | GLU | I | 16 | −5.037 | 44.223 | −25.627 | 1.00 | 27.46 | O |
| ATOM | 3462 | N | SER | I | 17 | −5.342 | 46.246 | −24.648 | 1.00 | 28.39 | N |
| ATOM | 3463 | CA | SER | I | 17 | −4.026 | 46.292 | −24.026 | 1.00 | 29.06 | C |
| ATOM | 3464 | CB | SER | I | 17 | −3.376 | 47.672 | −24.205 | 1.00 | 28.97 | C |
| ATOM | 3465 | OG | SER | I | 17 | −3.924 | 48.616 | −23.300 | 1.00 | 29.71 | O |
| ATOM | 3466 | C | SER | I | 17 | −4.108 | 45.892 | −22.543 | 1.00 | 28.98 | C |
| ATOM | 3467 | O | SER | I | 17 | −5.126 | 46.108 | −21.872 | 1.00 | 29.30 | O |
| ATOM | 3468 | N | LEU | I | 18 | −3.031 | 45.297 | −22.047 | 1.00 | 28.74 | N |
| ATOM | 3469 | CA | LEU | I | 18 | −2.982 | 44.806 | −20.673 | 1.00 | 28.36 | C |
| ATOM | 3470 | CB | LEU | I | 18 | −3.404 | 43.336 | −20.642 | 1.00 | 28.58 | C |
| ATOM | 3471 | CG | LEU | I | 18 | −3.082 | 42.404 | −19.480 | 1.00 | 28.49 | C |
| ATOM | 3472 | CD1 | LEU | I | 18 | −4.152 | 41.345 | −19.449 | 1.00 | 28.56 | C |
| ATOM | 3473 | CD2 | LEU | I | 18 | −1.714 | 41.758 | −19.673 | 1.00 | 30.06 | C |
| ATOM | 3474 | C | LEU | I | 18 | −1.599 | 44.982 | −20.055 | 1.00 | 27.56 | C |
| ATOM | 3475 | O | LEU | I | 18 | −0.586 | 44.741 | −20.704 | 1.00 | 27.12 | O |
| ATOM | 3476 | N | LYS | I | 19 | −1.580 | 45.396 | −18.795 | 1.00 | 27.01 | N |
| ATOM | 3477 | CA | LYS | I | 19 | −0.355 | 45.479 | −18.027 | 1.00 | 26.61 | C |
| ATOM | 3478 | CB | LYS | I | 19 | 0.045 | 46.940 | −17.801 | 1.00 | 26.97 | C |
| ATOM | 3479 | CG | LYS | I | 19 | 1.539 | 47.171 | −17.601 | 1.00 | 27.61 | C |
| ATOM | 3480 | CD | LYS | I | 19 | 1.786 | 48.498 | −16.883 | 1.00 | 29.42 | C |
| ATOM | 3481 | CE | LYS | I | 19 | 3.041 | 49.182 | −17.404 | 1.00 | 31.28 | C |
| ATOM | 3482 | NZ | LYS | I | 19 | 3.150 | 50.605 | −16.962 | 1.00 | 32.11 | N |
| ATOM | 3483 | C | LYS | I | 19 | −0.551 | 44.766 | −16.699 | 1.00 | 25.77 | C |
| ATOM | 3484 | O | LYS | I | 19 | −1.439 | 45.111 | −15.921 | 1.00 | 25.59 | O |
| ATOM | 3485 | N | ILE | I | 20 | 0.282 | 43.759 | −16.455 | 1.00 | 25.18 | N |
| ATOM | 3486 | CA | ILE | I | 20 | 0.257 | 43.008 | −15.191 | 1.00 | 24.00 | C |
| ATOM | 3487 | CB | ILE | I | 20 | 0.007 | 41.498 | −15.421 | 1.00 | 24.02 | C |
| ATOM | 3488 | CG1 | ILE | I | 20 | 1.134 | 40.862 | −16.237 | 1.00 | 22.96 | C |
| ATOM | 3489 | CD1 | ILE | I | 20 | 0.966 | 39.376 | −16.440 | 1.00 | 22.88 | C |
| ATOM | 3490 | CG2 | ILE | I | 20 | −1.350 | 41.292 | −16.092 | 1.00 | 23.03 | C |
| ATOM | 3491 | C | ILE | I | 20 | 1.549 | 43.241 | −14.420 | 1.00 | 23.54 | C |
| ATOM | 3492 | O | ILE | I | 20 | 2.603 | 43.456 | −15.024 | 1.00 | 23.33 | O |
| ATOM | 3493 | N | SER | I | 21 | 1.467 | 43.216 | −13.095 | 1.00 | 23.14 | N |
| ATOM | 3494 | CA | SER | I | 21 | 2.613 | 43.580 | −12.274 | 1.00 | 23.42 | C |
| ATOM | 3495 | CB | SER | I | 21 | 2.274 | 44.793 | −11.415 | 1.00 | 23.24 | C |
| ATOM | 3496 | OG | SER | I | 21 | 1.332 | 44.453 | −10.418 | 1.00 | 23.29 | O |
| ATOM | 3497 | C | SER | I | 21 | 3.151 | 42.454 | −11.393 | 1.00 | 23.88 | C |
| ATOM | 3498 | O | SER | I | 21 | 2.434 | 41.512 | −11.040 | 1.00 | 23.79 | O |
| ATOM | 3499 | N | CYS | I | 22 | 4.426 | 42.580 | −11.044 | 1.00 | 24.38 | N |
| ATOM | 3500 | CA | CYS | I | 22 | 5.114 | 41.669 | −10.138 | 1.00 | 25.27 | C |
| ATOM | 3501 | CB | CYS | I | 22 | 6.043 | 40.743 | −10.944 | 1.00 | 24.88 | C |
| ATOM | 3502 | SG | CYS | I | 22 | 7.035 | 39.571 | −9.977 | 1.00 | 24.60 | S |
| ATOM | 3503 | C | CYS | I | 22 | 5.902 | 42.523 | −9.120 | 1.00 | 26.15 | C |
| ATOM | 3504 | O | CYS | I | 22 | 6.920 | 43.131 | −9.459 | 1.00 | 26.35 | O |
| ATOM | 3505 | N | GLN | I | 23 | 5.415 | 42.604 | −7.886 | 1.00 | 26.88 | N |
| ATOM | 3506 | CA | GLN | I | 23 | 6.075 | 43.427 | −6.893 | 1.00 | 27.61 | C |
| ATOM | 3507 | CB | GLN | I | 23 | 5.069 | 44.208 | −6.044 | 1.00 | 28.17 | C |
| ATOM | 3508 | CG | GLN | I | 23 | 5.737 | 45.300 | −5.197 | 1.00 | 29.84 | C |
| ATOM | 3509 | CD | GLN | I | 23 | 4.753 | 46.286 | −4.613 | 1.00 | 32.41 | C |
| ATOM | 3510 | OE1 | GLN | I | 23 | 3.619 | 45.937 | −4.294 | 1.00 | 34.96 | O |
| ATOM | 3511 | NE2 | GLN | I | 23 | 5.190 | 47.532 | −4.452 | 1.00 | 33.62 | N |
| ATOM | 3512 | C | GLN | I | 23 | 7.014 | 42.611 | −6.019 | 1.00 | 27.75 | C |
| ATOM | 3513 | O | GLN | I | 23 | 6.627 | 41.590 | −5.455 | 1.00 | 27.43 | O |
| ATOM | 3514 | N | ALA | I | 24 | 8.257 | 43.067 | −5.932 | 1.00 | 28.06 | N |
| ATOM | 3515 | CA | ALA | I | 24 | 9.269 | 42.378 | −5.174 | 1.00 | 28.68 | C |
| ATOM | 3516 | CB | ALA | I | 24 | 10.589 | 42.430 | −5.893 | 1.00 | 28.68 | C |
| ATOM | 3517 | C | ALA | I | 24 | 9.385 | 42.981 | −3.786 | 1.00 | 29.36 | C |
| ATOM | 3518 | O | ALA | I | 24 | 9.579 | 44.180 | −3.626 | 1.00 | 29.27 | O |
| ATOM | 3519 | N | PHE | I | 25 | 9.234 | 42.127 | −2.784 | 1.00 | 30.66 | N |
| ATOM | 3520 | CA | PHE | I | 25 | 9.332 | 42.523 | −1.394 | 1.00 | 31.71 | C |
| ATOM | 3521 | CB | PHE | I | 25 | 8.134 | 42.018 | −0.621 | 1.00 | 31.30 | C |
| ATOM | 3522 | CG | PHE | I | 25 | 6.922 | 42.799 | −0.883 | 1.00 | 30.71 | C |
| ATOM | 3523 | CD1 | PHE | I | 25 | 6.616 | 43.892 | −0.090 | 1.00 | 30.41 | C |
| ATOM | 3524 | CE1 | PHE | I | 25 | 5.495 | 44.648 | −0.342 | 1.00 | 32.47 | C |
| ATOM | 3525 | CZ | PHE | I | 25 | 4.675 | 44.326 | −1.418 | 1.00 | 32.11 | C |
| ATOM | 3526 | CE2 | PHE | I | 25 | 4.995 | 43.237 | −2.235 | 1.00 | 32.17 | C |
| ATOM | 3527 | CD2 | PHE | I | 25 | 6.114 | 42.488 | −1.961 | 1.00 | 30.35 | C |
| ATOM | 3528 | C | PHE | I | 25 | 10.586 | 41.941 | −0.846 | 1.00 | 32.99 | C |
| ATOM | 3529 | O | PHE | I | 25 | 10.736 | 40.721 | −0.776 | 1.00 | 33.48 | O |
| ATOM | 3530 | N | GLY | I | 26 | 11.503 | 42.823 | −0.483 | 1.00 | 34.41 | N |
| ATOM | 3531 | CA | GLY | I | 26 | 12.822 | 42.399 | −0.077 | 1.00 | 36.23 | C |
| ATOM | 3532 | C | GLY | I | 26 | 13.872 | 43.399 | −0.492 | 1.00 | 37.33 | C |
| ATOM | 3533 | O | GLY | I | 26 | 13.614 | 44.601 | −0.540 | 1.00 | 37.58 | O |
| ATOM | 3534 | N | TYR | I | 27 | 15.048 | 42.882 | −0.822 | 1.00 | 38.21 | N |
| ATOM | 3535 | CA | TYR | I | 27 | 16.266 | 43.677 | −0.837 | 1.00 | 39.04 | C |
| ATOM | 3536 | CB | TYR | I | 27 | 17.162 | 43.175 | 0.307 | 1.00 | 40.21 | C |
| ATOM | 3537 | CG | TYR | I | 27 | 18.475 | 43.898 | 0.465 | 1.00 | 44.47 | C |
| ATOM | 3538 | CD1 | TYR | I | 27 | 18.600 | 44.960 | 1.368 | 1.00 | 48.18 | C |
| ATOM | 3539 | CE1 | TYR | I | 27 | 19.825 | 45.634 | 1.527 | 1.00 | 51.28 | C |
| ATOM | 3540 | CZ | TYR | I | 27 | 20.938 | 45.236 | 0.770 | 1.00 | 52.22 | C |
| ATOM | 3541 | OH | TYR | I | 27 | 22.141 | 45.901 | 0.918 | 1.00 | 53.77 | O |
| ATOM | 3542 | CE2 | TYR | I | 27 | 20.838 | 44.174 | −0.133 | 1.00 | 51.09 | C |
| ATOM | 3543 | CD2 | TYR | I | 27 | 19.608 | 43.508 | −0.276 | 1.00 | 48.65 | C |
| ATOM | 3544 | C | TYR | I | 27 | 16.988 | 43.579 | −2.181 | 1.00 | 37.84 | C |
| ATOM | 3545 | O | TYR | I | 27 | 17.080 | 42.500 | −2.751 | 1.00 | 38.15 | O |
| ATOM | 3546 | N | GLY | I | 28 | 17.484 | 44.712 | −2.679 | 1.00 | 36.77 | N |
| ATOM | 3547 | CA | GLY | I | 28 | 18.342 | 44.760 | −3.879 | 1.00 | 34.85 | C |
| ATOM | 3548 | C | GLY | I | 28 | 17.671 | 44.464 | −5.211 | 1.00 | 33.54 | C |
| ATOM | 3549 | O | GLY | I | 28 | 18.280 | 43.869 | −6.105 | 1.00 | 33.69 | O |
| ATOM | 3550 | N | PHE | I | 29 | 16.415 | 44.883 | −5.333 | 1.00 | 31.87 | N |
| ATOM | 3551 | CA | PHE | I | 29 | 15.590 | 44.690 | −6.525 | 1.00 | 30.17 | C |
| ATOM | 3552 | CB | PHE | I | 29 | 14.354 | 45.587 | −6.398 | 1.00 | 29.98 | C |
| ATOM | 3553 | CG | PHE | I | 29 | 13.480 | 45.613 | −7.613 | 1.00 | 29.38 | C |
| ATOM | 3554 | CD1 | PHE | I | 29 | 12.752 | 44.491 | −7.992 | 1.00 | 28.55 | C |
| ATOM | 3555 | CE1 | PHE | I | 29 | 11.935 | 44.519 | −9.116 | 1.00 | 28.48 | C |
| ATOM | 3556 | CZ | PHE | I | 29 | 11.817 | 45.679 | −9.863 | 1.00 | 28.38 | C |
| ATOM | 3557 | CE2 | PHE | I | 29 | 12.525 | 46.812 | −9.491 | 1.00 | 29.49 | C |
| ATOM | 3558 | CD2 | PHE | I | 29 | 13.356 | 46.775 | −8.366 | 1.00 | 29.62 | C |
| ATOM | 3559 | C | PHE | I | 29 | 16.310 | 44.930 | −7.864 | 1.00 | 29.53 | C |
| ATOM | 3560 | O | PHE | I | 29 | 16.150 | 44.162 | −8.819 | 1.00 | 28.78 | O |
| ATOM | 3561 | N | ILE | I | 30 | 17.102 | 45.998 | −7.922 | 1.00 | 28.92 | N |
| ATOM | 3562 | CA | ILE | I | 30 | 17.799 | 46.383 | −9.143 | 1.00 | 28.38 | C |
| ATOM | 3563 | CB | ILE | I | 30 | 18.501 | 47.747 | −8.982 | 1.00 | 28.73 | C |
| ATOM | 3564 | CG1 | ILE | I | 30 | 19.321 | 47.767 | −7.692 | 1.00 | 28.95 | C |
| ATOM | 3565 | CD1 | ILE | I | 30 | 20.738 | 48.229 | −7.896 | 1.00 | 30.22 | C |
| ATOM | 3566 | CG2 | ILE | I | 30 | 17.479 | 48.893 | −8.999 | 1.00 | 27.50 | C |
| ATOM | 3567 | C | ILE | I | 30 | 18.817 | 45.337 | −9.615 | 1.00 | 28.21 | C |
| ATOM | 3568 | O | ILE | I | 30 | 19.151 | 45.297 | −10.800 | 1.00 | 28.44 | O |
| ATOM | 3569 | N | ASN | I | 31 | 19.295 | 44.508 | −8.684 | 1.00 | 27.73 | N |
| ATOM | 3570 | CA | ASN | I | 31 | 20.230 | 43.416 | −8.964 | 1.00 | 27.46 | C |
| ATOM | 3571 | CB | ASN | I | 31 | 20.986 | 42.993 | −7.687 | 1.00 | 28.05 | C |
| ATOM | 3572 | CG | ASN | I | 31 | 21.889 | 44.102 | −7.132 | 1.00 | 30.64 | C |
| ATOM | 3573 | OD1 | ASN | I | 31 | 21.788 | 44.485 | −5.953 | 1.00 | 31.82 | O |
| ATOM | 3574 | ND2 | ASN | I | 31 | 22.778 | 44.624 | −7.984 | 1.00 | 32.80 | N |
| ATOM | 3575 | C | ASN | I | 31 | 19.559 | 42.182 | −9.548 | 1.00 | 26.49 | C |
| ATOM | 3576 | O | ASN | I | 31 | 20.240 | 41.328 | −10.102 | 1.00 | 26.66 | O |
| ATOM | 3577 | N | TYR | I | 32 | 18.237 | 42.092 | −9.411 | 1.00 | 25.43 | N |
| ATOM | 3578 | CA | TYR | I | 32 | 17.452 | 40.920 | −9.835 | 1.00 | 24.30 | C |
| ATOM | 3579 | CB | TYR | I | 32 | 15.981 | 41.060 | −9.417 | 1.00 | 23.97 | C |
| ATOM | 3580 | CG | TYR | I | 32 | 15.680 | 41.011 | −7.937 | 1.00 | 23.56 | C |
| ATOM | 3581 | CD1 | TYR | I | 32 | 16.677 | 41.211 | −6.974 | 1.00 | 25.25 | C |
| ATOM | 3582 | CE1 | TYR | I | 32 | 16.378 | 41.184 | −5.601 | 1.00 | 25.39 | C |
| ATOM | 3583 | CZ | TYR | I | 32 | 15.064 | 40.968 | −5.204 | 1.00 | 25.61 | C |
| ATOM | 3584 | OH | TYR | I | 32 | 14.738 | 40.936 | −3.871 | 1.00 | 26.30 | O |
| ATOM | 3585 | CE2 | TYR | I | 32 | 14.063 | 40.788 | −6.151 | 1.00 | 24.12 | C |
| ATOM | 3586 | CD2 | TYR | I | 32 | 14.378 | 40.815 | −7.500 | 1.00 | 22.75 | C |
| ATOM | 3587 | C | TYR | I | 32 | 17.443 | 40.660 | −11.332 | 1.00 | 23.77 | C |
| ATOM | 3588 | O | TYR | I | 32 | 17.370 | 41.590 | −12.139 | 1.00 | 23.89 | O |
| ATOM | 3589 | N | LEU | I | 33 | 17.480 | 39.383 | −11.686 | 1.00 | 23.11 | N |
| ATOM | 3590 | CA | LEU | I | 33 | 17.085 | 38.946 | −13.008 | 1.00 | 22.77 | C |
| ATOM | 3591 | CB | LEU | I | 33 | 18.051 | 37.886 | −13.521 | 1.00 | 23.10 | C |
| ATOM | 3592 | CG | LEU | I | 33 | 19.485 | 38.436 | −13.633 | 1.00 | 25.01 | C |
| ATOM | 3593 | CD1 | LEU | I | 33 | 20.474 | 37.347 | −14.031 | 1.00 | 25.06 | C |
| ATOM | 3594 | CD2 | LEU | I | 33 | 19.567 | 39.640 | −14.597 | 1.00 | 24.81 | C |
| ATOM | 3595 | C | LEU | I | 33 | 15.668 | 38.396 | −12.927 | 1.00 | 22.28 | C |
| ATOM | 3596 | O | LEU | I | 33 | 15.455 | 37.275 | −12.468 | 1.00 | 22.58 | O |
| ATOM | 3597 | N | ILE | I | 34 | 14.694 | 39.195 | −13.343 | 1.00 | 21.34 | N |
| ATOM | 3598 | CA | ILE | I | 34 | 13.296 | 38.775 | −13.292 | 1.00 | 20.63 | C |
| ATOM | 3599 | CB | ILE | I | 34 | 12.373 | 39.926 | −12.847 | 1.00 | 20.60 | C |
| ATOM | 3600 | CG1 | ILE | I | 34 | 12.729 | 40.350 | −11.420 | 1.00 | 21.70 | C |
| ATOM | 3601 | CD1 | ILE | I | 34 | 12.025 | 41.592 | −10.930 | 1.00 | 21.45 | C |
| ATOM | 3602 | CG2 | ILE | I | 34 | 10.911 | 39.520 | −12.915 | 1.00 | 20.58 | C |
| ATOM | 3603 | C | ILE | I | 34 | 12.869 | 38.171 | −14.638 | 1.00 | 20.19 | C |
| ATOM | 3604 | O | ILE | I | 34 | 13.083 | 38.758 | −15.705 | 1.00 | 20.04 | O |
| ATOM | 3605 | N | GLU | I | 35 | 12.309 | 36.971 | −14.571 | 1.00 | 19.70 | N |
| ATOM | 3606 | CA | GLU | I | 35 | 11.871 | 36.249 | −15.754 | 1.00 | 19.22 | C |
| ATOM | 3607 | CB | GLU | I | 35 | 12.435 | 34.837 | −15.780 | 1.00 | 19.38 | C |
| ATOM | 3608 | CG | GLU | I | 35 | 13.937 | 34.754 | −15.833 | 1.00 | 19.74 | C |
| ATOM | 3609 | CD | GLU | I | 35 | 14.412 | 33.326 | −15.771 | 1.00 | 21.73 | C |
| ATOM | 3610 | OE1 | GLU | I | 35 | 15.078 | 32.951 | −14.783 | 1.00 | 22.41 | O |
| ATOM | 3611 | OE2 | GLU | I | 35 | 14.110 | 32.571 | −16.715 | 1.00 | 23.01 | O |
| ATOM | 3612 | C | GLU | I | 35 | 10.365 | 36.174 | −15.772 | 1.00 | 18.87 | C |
| ATOM | 3613 | O | GLU | I | 35 | 9.714 | 36.211 | −14.725 | 1.00 | 18.53 | O |
| ATOM | 3614 | N | TRP | I | 36 | 9.814 | 36.099 | −16.976 | 1.00 | 18.78 | N |
| ATOM | 3615 | CA | TRP | I | 36 | 8.389 | 35.888 | −17.135 | 1.00 | 18.74 | C |
| ATOM | 3616 | CB | TRP | I | 36 | 7.733 | 37.051 | −17.839 | 1.00 | 18.08 | C |
| ATOM | 3617 | CG | TRP | I | 36 | 7.660 | 38.286 | −17.027 | 1.00 | 18.41 | C |
| ATOM | 3618 | CD1 | TRP | I | 36 | 8.617 | 39.258 | −16.929 | 1.00 | 18.34 | C |
| ATOM | 3619 | NE1 | TRP | I | 36 | 8.176 | 40.268 | −16.103 | 1.00 | 18.22 | N |
| ATOM | 3620 | CE2 | TRP | I | 36 | 6.920 | 39.962 | −15.650 | 1.00 | 15.80 | C |
| ATOM | 3621 | CD2 | TRP | I | 36 | 6.562 | 38.716 | −16.205 | 1.00 | 16.37 | C |
| ATOM | 3622 | CE3 | TRP | I | 36 | 5.317 | 38.172 | −15.892 | 1.00 | 14.82 | C |
| ATOM | 3623 | CZ3 | TRP | I | 36 | 4.473 | 38.878 | −15.049 | 1.00 | 16.56 | C |
| ATOM | 3624 | CH2 | TRP | I | 36 | 4.858 | 40.116 | −14.509 | 1.00 | 17.46 | C |
| ATOM | 3625 | CZ2 | TRP | I | 36 | 6.079 | 40.673 | −14.803 | 1.00 | 16.72 | C |
| ATOM | 3626 | C | TRP | I | 36 | 8.160 | 34.623 | −17.915 | 1.00 | 19.05 | C |
| ATOM | 3627 | O | TRP | I | 36 | 8.828 | 34.366 | −18.928 | 1.00 | 19.18 | O |
| ATOM | 3628 | N | ILE | I | 37 | 7.200 | 33.848 | −17.426 | 1.00 | 19.50 | N |
| ATOM | 3629 | CA | ILE | I | 37 | 6.934 | 32.509 | −17.904 | 1.00 | 20.10 | C |
| ATOM | 3630 | CB | ILE | I | 37 | 7.642 | 31.480 | −17.004 | 1.00 | 19.75 | C |
| ATOM | 3631 | CG1 | ILE | I | 37 | 9.161 | 31.589 | −17.190 | 1.00 | 19.90 | C |
| ATOM | 3632 | CD1 | ILE | I | 37 | 9.986 | 31.002 | −16.054 | 1.00 | 20.86 | C |
| ATOM | 3633 | CG2 | ILE | I | 37 | 7.122 | 30.054 | −17.270 | 1.00 | 19.25 | C |
| ATOM | 3634 | C | ILE | I | 37 | 5.429 | 32.271 | −17.890 | 1.00 | 20.95 | C |
| ATOM | 3635 | O | ILE | I | 37 | 4.761 | 32.554 | −16.903 | 1.00 | 20.86 | O |
| ATOM | 3636 | N | ARG | I | 38 | 4.895 | 31.767 | −18.991 | 1.00 | 22.26 | N |
| ATOM | 3637 | CA | ARG | I | 38 | 3.470 | 31.478 | −19.045 | 1.00 | 23.75 | C |
| ATOM | 3638 | CB | ARG | I | 38 | 2.757 | 32.379 | −20.057 | 1.00 | 23.97 | C |
| ATOM | 3639 | CG | ARG | I | 38 | 2.976 | 31.980 | −21.516 | 1.00 | 25.83 | C |
| ATOM | 3640 | CD | ARG | I | 38 | 1.891 | 32.574 | −22.393 | 1.00 | 28.79 | C |
| ATOM | 3641 | NE | ARG | I | 38 | 1.994 | 32.101 | −23.769 | 1.00 | 31.25 | N |
| ATOM | 3642 | CZ | ARG | I | 38 | 1.442 | 32.706 | −24.818 | 1.00 | 31.88 | C |
| ATOM | 3643 | NH1 | ARG | I | 38 | 0.744 | 33.824 | −24.668 | 1.00 | 31.62 | N |
| ATOM | 3644 | NH2 | ARG | I | 38 | 1.603 | 32.189 | −26.027 | 1.00 | 32.28 | N |
| ATOM | 3645 | C | ARG | I | 38 | 3.184 | 30.000 | −19.327 | 1.00 | 24.21 | C |
| ATOM | 3646 | O | ARG | I | 38 | 3.955 | 29.304 | −19.984 | 1.00 | 23.28 | O |
| ATOM | 3647 | N | GLN | I | 39 | 2.052 | 29.552 | −18.811 | 1.00 | 25.47 | N |
| ATOM | 3648 | CA | GLN | I | 39 | 1.662 | 28.168 | −18.873 | 1.00 | 26.87 | C |
| ATOM | 3649 | CB | GLN | I | 39 | 1.857 | 27.528 | −17.496 | 1.00 | 26.43 | C |
| ATOM | 3650 | CG | GLN | I | 39 | 1.359 | 26.097 | −17.361 | 1.00 | 24.36 | C |
| ATOM | 3651 | CD | GLN | I | 39 | 1.937 | 25.409 | −16.143 | 1.00 | 22.11 | C |
| ATOM | 3652 | OE1 | GLN | I | 39 | 1.697 | 25.827 | −15.013 | 1.00 | 22.46 | O |
| ATOM | 3653 | NE2 | GLN | I | 39 | 2.726 | 24.369 | −16.367 | 1.00 | 18.79 | N |
| ATOM | 3654 | C | GLN | I | 39 | 0.198 | 28.116 | −19.270 | 1.00 | 28.66 | C |
| ATOM | 3655 | O | GLN | I | 39 | −0.668 | 28.614 | −18.538 | 1.00 | 28.39 | O |
| ATOM | 3656 | N | MET | I | 40 | −0.071 | 27.534 | −20.439 | 1.00 | 31.04 | N |
| ATOM | 3657 | CA | MET | I | 40 | −1.442 | 27.185 | −20.811 | 1.00 | 33.46 | C |
| ATOM | 3658 | CB | MET | I | 40 | −1.551 | 26.807 | −22.288 | 1.00 | 34.15 | C |
| ATOM | 3659 | CG | MET | I | 40 | −1.916 | 27.943 | −23.213 | 1.00 | 36.74 | C |
| ATOM | 3660 | SD | MET | I | 40 | −0.467 | 28.839 | −23.818 | 1.00 | 44.71 | S |
| ATOM | 3661 | CE | MET | I | 40 | −0.933 | 29.011 | −25.547 | 1.00 | 42.55 | C |
| ATOM | 3662 | C | MET | I | 40 | −1.869 | 26.012 | −19.938 | 1.00 | 34.12 | C |
| ATOM | 3663 | O | MET | I | 40 | −1.055 | 25.139 | −19.640 | 1.00 | 33.89 | O |
| ATOM | 3664 | N | PRO | I | 41 | −3.142 | 25.987 | −19.521 | 1.00 | 35.04 | N |
| ATOM | 3665 | CA | PRO | I | 41 | −3.584 | 24.933 | −18.591 | 1.00 | 35.59 | C |
| ATOM | 3666 | CB | PRO | I | 41 | −5.078 | 25.244 | −18.374 | 1.00 | 35.61 | C |
| ATOM | 3667 | CG | PRO | I | 41 | −5.486 | 26.075 | −19.571 | 1.00 | 36.28 | C |
| ATOM | 3668 | CD | PRO | I | 41 | −4.244 | 26.871 | −19.946 | 1.00 | 35.65 | C |
| ATOM | 3669 | C | PRO | I | 41 | −3.346 | 23.506 | −19.140 | 1.00 | 35.79 | C |
| ATOM | 3670 | O | PRO | I | 41 | −3.771 | 23.179 | −20.254 | 1.00 | 35.97 | O |
| ATOM | 3671 | N | GLY | I | 42 | −2.626 | 22.690 | −18.372 | 1.00 | 35.98 | N |
| ATOM | 3672 | CA | GLY | I | 42 | −2.244 | 21.340 | −18.800 | 1.00 | 36.07 | C |
| ATOM | 3673 | C | GLY | I | 42 | −1.085 | 21.281 | −19.787 | 1.00 | 36.21 | C |
| ATOM | 3674 | O | GLY | I | 42 | −0.674 | 20.189 | −20.211 | 1.00 | 36.18 | O |
| ATOM | 3675 | N | GLN | I | 43 | −0.564 | 22.454 | −20.159 | 1.00 | 35.94 | N |
| ATOM | 3676 | CA | GLN | I | 43 | 0.619 | 22.558 | −21.015 | 1.00 | 35.47 | C |
| ATOM | 3677 | CB | GLN | I | 43 | 0.425 | 23.623 | −22.102 | 1.00 | 35.82 | C |
| ATOM | 3678 | CG | GLN | I | 43 | −0.060 | 23.074 | −23.440 | 1.00 | 38.81 | C |
| ATOM | 3679 | CD | GLN | I | 43 | −1.396 | 22.346 | −23.324 | 1.00 | 41.95 | C |
| ATOM | 3680 | OE1 | GLN | I | 43 | −2.336 | 22.834 | −22.676 | 1.00 | 43.53 | O |
| ATOM | 3681 | NE2 | GLN | I | 43 | −1.480 | 21.169 | −23.941 | 1.00 | 41.42 | N |
| ATOM | 3682 | C | GLN | I | 43 | 1.883 | 22.860 | −20.217 | 1.00 | 34.36 | C |
| ATOM | 3683 | O | GLN | I | 43 | 1.851 | 23.000 | −18.991 | 1.00 | 33.91 | O |
| ATOM | 3684 | N | GLY | I | 44 | 2.997 | 22.948 | −20.938 | 1.00 | 33.28 | N |
| ATOM | 3685 | CA | GLY | I | 44 | 4.278 | 23.258 | −20.337 | 1.00 | 31.72 | C |
| ATOM | 3686 | C | GLY | I | 44 | 4.525 | 24.743 | −20.172 | 1.00 | 30.62 | C |
| ATOM | 3687 | O | GLY | I | 44 | 3.632 | 25.579 | −20.338 | 1.00 | 30.39 | O |
| ATOM | 3688 | N | LEU | I | 45 | 5.767 | 25.052 | −19.839 | 1.00 | 29.77 | N |
| ATOM | 3689 | CA | LEU | I | 45 | 6.185 | 26.411 | −19.580 | 1.00 | 28.95 | C |
| ATOM | 3690 | CB | LEU | I | 45 | 7.182 | 26.431 | −18.426 | 1.00 | 28.69 | C |
| ATOM | 3691 | CG | LEU | I | 45 | 6.735 | 25.625 | −17.206 | 1.00 | 28.17 | C |
| ATOM | 3692 | CD1 | LEU | I | 45 | 7.900 | 25.405 | −16.273 | 1.00 | 27.95 | C |
| ATOM | 3693 | CD2 | LEU | I | 45 | 5.583 | 26.312 | −16.488 | 1.00 | 28.09 | C |
| ATOM | 3694 | C | LEU | I | 45 | 6.781 | 27.038 | −20.831 | 1.00 | 28.58 | C |
| ATOM | 3695 | O | LEU | I | 45 | 7.642 | 26.450 | −21.501 | 1.00 | 28.72 | O |
| ATOM | 3696 | N | GLU | I | 46 | 6.277 | 28.218 | −21.164 | 1.00 | 27.88 | N |
| ATOM | 3697 | CA | GLU | I | 46 | 6.820 | 29.007 | −22.255 | 1.00 | 27.36 | C |
| ATOM | 3698 | CB | GLU | I | 46 | 5.709 | 29.486 | −23.202 | 1.00 | 27.77 | C |
| ATOM | 3699 | CG | GLU | I | 46 | 4.930 | 28.384 | −23.916 | 1.00 | 30.74 | C |
| ATOM | 3700 | CD | GLU | I | 46 | 4.012 | 28.936 | −25.020 | 1.00 | 35.24 | C |
| ATOM | 3701 | OE1 | GLU | I | 46 | 2.770 | 28.803 | −24.880 | 1.00 | 35.68 | O |
| ATOM | 3702 | OE2 | GLU | I | 46 | 4.540 | 29.513 | −26.015 | 1.00 | 36.70 | O |
| ATOM | 3703 | C | GLU | I | 46 | 7.536 | 30.202 | −21.643 | 1.00 | 26.03 | C |
| ATOM | 3704 | O | GLU | I | 46 | 6.946 | 30.971 | −20.878 | 1.00 | 25.83 | O |
| ATOM | 3705 | N | TRP | I | 47 | 8.806 | 30.344 | −21.986 | 1.00 | 24.61 | N |
| ATOM | 3706 | CA | TRP | I | 47 | 9.629 | 31.412 | −21.472 | 1.00 | 23.77 | C |
| ATOM | 3707 | CB | TRP | I | 47 | 11.089 | 31.007 | −21.623 | 1.00 | 23.56 | C |
| ATOM | 3708 | CG | TRP | I | 47 | 12.058 | 31.876 | −20.902 | 1.00 | 23.96 | C |
| ATOM | 3709 | CD1 | TRP | I | 47 | 12.354 | 31.844 | −19.567 | 1.00 | 24.01 | C |
| ATOM | 3710 | NE1 | TRP | I | 47 | 13.314 | 32.779 | −19.270 | 1.00 | 24.00 | N |
| ATOM | 3711 | CE2 | TRP | I | 47 | 13.671 | 33.431 | −20.422 | 1.00 | 24.82 | C |
| ATOM | 3712 | CD2 | TRP | I | 47 | 12.903 | 32.881 | −21.476 | 1.00 | 23.81 | C |
| ATOM | 3713 | CE3 | TRP | I | 47 | 13.082 | 33.378 | −22.775 | 1.00 | 22.56 | C |
| ATOM | 3714 | CZ3 | TRP | I | 47 | 14.005 | 34.403 | −22.982 | 1.00 | 22.33 | C |
| ATOM | 3715 | CH2 | TRP | I | 47 | 14.755 | 34.931 | −21.915 | 1.00 | 23.78 | C |
| ATOM | 3716 | CZ2 | TRP | I | 47 | 14.604 | 34.461 | −20.630 | 1.00 | 24.74 | C |
| ATOM | 3717 | C | TRP | I | 47 | 9.338 | 32.682 | −22.260 | 1.00 | 23.40 | C |
| ATOM | 3718 | O | TRP | I | 47 | 9.514 | 32.695 | −23.473 | 1.00 | 23.54 | O |
| ATOM | 3719 | N | ILE | I | 48 | 8.884 | 33.744 | −21.593 | 1.00 | 22.49 | N |
| ATOM | 3720 | CA | ILE | I | 48 | 8.588 | 34.989 | −22.310 | 1.00 | 21.87 | C |
| ATOM | 3721 | CB | ILE | I | 48 | 7.444 | 35.792 | −21.666 | 1.00 | 21.82 | C |
| ATOM | 3722 | CG1 | ILE | I | 48 | 6.140 | 35.000 | −21.750 | 1.00 | 20.12 | C |
| ATOM | 3723 | CD1 | ILE | I | 48 | 5.085 | 35.472 | −20.803 | 1.00 | 19.18 | C |
| ATOM | 3724 | CG2 | ILE | I | 48 | 7.295 | 37.159 | −22.337 | 1.00 | 21.12 | C |
| ATOM | 3725 | C | ILE | I | 48 | 9.840 | 35.841 | −22.465 | 1.00 | 21.93 | C |
| ATOM | 3726 | O | ILE | I | 48 | 10.200 | 36.227 | −23.569 | 1.00 | 22.55 | O |
| ATOM | 3727 | N | GLY | I | 49 | 10.508 | 36.128 | −21.361 | 1.00 | 21.58 | N |
| ATOM | 3728 | CA | GLY | I | 49 | 11.762 | 36.837 | −21.420 | 1.00 | 20.92 | C |
| ATOM | 3729 | C | GLY | I | 49 | 12.331 | 37.154 | −20.065 | 1.00 | 20.71 | C |
| ATOM | 3730 | O | GLY | I | 49 | 11.763 | 36.789 | −19.039 | 1.00 | 20.50 | O |
| ATOM | 3731 | N | LEU | I | 50 | 13.451 | 37.869 | −20.073 | 1.00 | 20.70 | N |
| ATOM | 3732 | CA | LEU | I | 50 | 14.131 | 38.258 | −18.853 | 1.00 | 20.53 | C |
| ATOM | 3733 | CB | LEU | I | 50 | 15.404 | 37.428 | −18.702 | 1.00 | 19.97 | C |
| ATOM | 3734 | CG | LEU | I | 50 | 16.546 | 37.857 | −17.789 | 1.00 | 20.92 | C |
| ATOM | 3735 | CD1 | LEU | I | 50 | 17.258 | 36.605 | −17.243 | 1.00 | 22.10 | C |
| ATOM | 3736 | CD2 | LEU | I | 50 | 17.545 | 38.815 | −18.511 | 1.00 | 20.03 | C |
| ATOM | 3737 | C | LEU | I | 50 | 14.426 | 39.767 | −18.850 | 1.00 | 20.83 | C |
| ATOM | 3738 | O | LEU | I | 50 | 14.822 | 40.333 | −19.882 | 1.00 | 20.47 | O |
| ATOM | 3739 | N | ILE | I | 51 | 14.229 | 40.391 | −17.684 | 1.00 | 20.76 | N |
| ATOM | 3740 | CA | ILE | I | 51 | 14.616 | 41.784 | −17.424 | 1.00 | 20.53 | C |
| ATOM | 3741 | CB | ILE | I | 51 | 13.374 | 42.708 | −17.299 | 1.00 | 20.67 | C |
| ATOM | 3742 | CG1 | ILE | I | 51 | 13.808 | 44.179 | −17.272 | 1.00 | 21.11 | C |
| ATOM | 3743 | CD1 | ILE | I | 51 | 12.680 | 45.172 | −17.567 | 1.00 | 21.89 | C |
| ATOM | 3744 | CG2 | ILE | I | 51 | 12.502 | 42.327 | −16.069 | 1.00 | 19.95 | C |
| ATOM | 3745 | C | ILE | I | 51 | 15.504 | 41.978 | −16.178 | 1.00 | 20.58 | C |
| ATOM | 3746 | O | ILE | I | 51 | 15.271 | 41.377 | −15.124 | 1.00 | 20.46 | O |
| ATOM | 3747 | N | ASN | I | 52 | 16.524 | 42.822 | −16.313 | 1.00 | 20.90 | N |
| ATOM | 3748 | CA | ASN | I | 52 | 17.237 | 43.379 | −15.156 | 1.00 | 20.64 | C |
| ATOM | 3749 | CB | ASN | I | 52 | 18.750 | 43.284 | −15.341 | 1.00 | 20.41 | C |
| ATOM | 3750 | CG | ASN | I | 52 | 19.512 | 43.985 | −14.247 | 1.00 | 20.63 | C |
| ATOM | 3751 | OD1 | ASN | I | 52 | 19.383 | 43.652 | −13.064 | 1.00 | 21.76 | O |
| ATOM | 3752 | ND2 | ASN | I | 52 | 20.307 | 44.968 | −14.628 | 1.00 | 20.12 | N |
| ATOM | 3753 | C | ASN | I | 52 | 16.799 | 44.830 | −14.906 | 1.00 | 20.70 | C |
| ATOM | 3754 | O | ASN | I | 52 | 17.110 | 45.715 | −15.700 | 1.00 | 20.85 | O |
| ATOM | 3755 | N | PRO | I | 52A | 16.059 | 45.074 | −13.805 | 1.00 | 20.83 | N |
| ATOM | 3756 | CA | PRO | I | 52A | 15.429 | 46.371 | −13.584 | 1.00 | 21.21 | C |
| ATOM | 3757 | CB | PRO | I | 52A | 14.755 | 46.208 | −12.219 | 1.00 | 20.81 | C |
| ATOM | 3758 | CG | PRO | I | 52A | 14.590 | 44.787 | −12.041 | 1.00 | 20.41 | C |
| ATOM | 3759 | CD | PRO | I | 52A | 15.771 | 44.149 | −12.697 | 1.00 | 20.72 | C |
| ATOM | 3760 | C | PRO | I | 52A | 16.424 | 47.534 | −13.547 | 1.00 | 22.10 | C |
| ATOM | 3761 | O | PRO | I | 52A | 16.160 | 48.589 | −14.135 | 1.00 | 22.38 | O |
| ATOM | 3762 | N | GLY | I | 53 | 17.557 | 47.346 | −12.870 | 1.00 | 22.61 | N |
| ATOM | 3763 | CA | GLY | I | 53 | 18.567 | 48.403 | −12.794 | 1.00 | 23.12 | C |
| ATOM | 3764 | C | GLY | I | 53 | 19.026 | 48.928 | −14.147 | 1.00 | 23.22 | C |
| ATOM | 3765 | O | GLY | I | 53 | 18.973 | 50.129 | −14.396 | 1.00 | 23.22 | O |
| ATOM | 3766 | N | SER | I | 54 | 19.457 | 48.021 | −15.023 | 1.00 | 23.34 | N |
| ATOM | 3767 | CA | SER | I | 54 | 19.945 | 48.386 | −16.346 | 1.00 | 23.65 | C |
| ATOM | 3768 | CB | SER | I | 54 | 21.003 | 47.388 | −16.807 | 1.00 | 23.76 | C |
| ATOM | 3769 | OG | SER | I | 54 | 20.433 | 46.109 | −17.049 | 1.00 | 24.26 | O |
| ATOM | 3770 | C | SER | I | 54 | 18.854 | 48.489 | −17.417 | 1.00 | 24.18 | C |
| ATOM | 3771 | O | SER | I | 54 | 19.126 | 48.984 | −18.519 | 1.00 | 23.95 | O |
| ATOM | 3772 | N | ASP | I | 55 | 17.635 | 48.035 | −17.082 | 1.00 | 24.56 | N |
| ATOM | 3773 | CA | ASP | I | 55 | 16.562 | 47.741 | −18.055 | 1.00 | 24.76 | C |
| ATOM | 3774 | CB | ASP | I | 55 | 15.839 | 49.018 | −18.575 | 1.00 | 25.70 | C |
| ATOM | 3775 | CG | ASP | I | 55 | 14.955 | 49.713 | −17.488 | 1.00 | 28.89 | C |
| ATOM | 3776 | OD1 | ASP | I | 55 | 14.553 | 50.892 | −17.682 | 1.00 | 31.43 | O |
| ATOM | 3777 | OD2 | ASP | I | 55 | 14.655 | 49.105 | −16.432 | 1.00 | 32.05 | O |
| ATOM | 3778 | C | ASP | I | 55 | 17.006 | 46.797 | −19.200 | 1.00 | 24.02 | C |
| ATOM | 3779 | O | ASP | I | 55 | 16.384 | 46.763 | −20.255 | 1.00 | 23.68 | O |
| ATOM | 3780 | N | TYR | I | 56 | 18.068 | 46.022 | −18.986 | 1.00 | 23.83 | N |
| ATOM | 3781 | CA | TYR | I | 56 | 18.436 | 44.974 | −19.943 | 1.00 | 23.94 | C |
| ATOM | 3782 | CB | TYR | I | 56 | 19.725 | 44.242 | −19.532 | 1.00 | 24.12 | C |
| ATOM | 3783 | CG | TYR | I | 56 | 20.074 | 43.055 | −20.425 | 1.00 | 24.83 | C |
| ATOM | 3784 | CD1 | TYR | I | 56 | 20.874 | 43.220 | −21.558 | 1.00 | 26.56 | C |
| ATOM | 3785 | CE1 | TYR | I | 56 | 21.200 | 42.132 | −22.389 | 1.00 | 25.54 | C |
| ATOM | 3786 | CZ | TYR | I | 56 | 20.709 | 40.874 | −22.086 | 1.00 | 25.62 | C |
| ATOM | 3787 | OH | TYR | I | 56 | 21.021 | 39.814 | −22.913 | 1.00 | 25.01 | O |
| ATOM | 3788 | CE2 | TYR | I | 56 | 19.907 | 40.687 | −20.969 | 1.00 | 24.06 | C |
| ATOM | 3789 | CD2 | TYR | I | 56 | 19.594 | 41.770 | −20.148 | 1.00 | 24.48 | C |
| ATOM | 3790 | C | TYR | I | 56 | 17.298 | 43.962 | −20.045 | 1.00 | 24.05 | C |
| ATOM | 3791 | O | TYR | I | 56 | 16.760 | 43.516 | −19.025 | 1.00 | 23.65 | O |
| ATOM | 3792 | N | THR | I | 57 | 16.947 | 43.603 | −21.275 | 1.00 | 24.10 | N |
| ATOM | 3793 | CA | THR | I | 57 | 15.885 | 42.648 | −21.527 | 1.00 | 24.51 | C |
| ATOM | 3794 | CB | THR | I | 57 | 14.590 | 43.325 | −22.026 | 1.00 | 24.54 | C |
| ATOM | 3795 | OG1 | THR | I | 57 | 14.856 | 44.008 | −23.255 | 1.00 | 24.83 | O |
| ATOM | 3796 | CG2 | THR | I | 57 | 14.009 | 44.303 | −21.006 | 1.00 | 23.98 | C |
| ATOM | 3797 | C | THR | I | 57 | 16.310 | 41.681 | −22.608 | 1.00 | 24.83 | C |
| ATOM | 3798 | O | THR | I | 57 | 16.932 | 42.077 | −23.580 | 1.00 | 25.19 | O |
| ATOM | 3799 | N | ASN | I | 58 | 15.965 | 40.412 | −22.436 | 1.00 | 25.20 | N |
| ATOM | 3800 | CA | ASN | I | 58 | 16.126 | 39.425 | −23.481 | 1.00 | 25.53 | C |
| ATOM | 3801 | CB | ASN | I | 58 | 17.219 | 38.424 | −23.120 | 1.00 | 25.61 | C |
| ATOM | 3802 | CG | ASN | I | 58 | 17.536 | 37.446 | −24.259 | 1.00 | 25.80 | C |
| ATOM | 3803 | OD1 | ASN | I | 58 | 16.697 | 36.632 | −24.654 | 1.00 | 27.94 | O |
| ATOM | 3804 | ND2 | ASN | I | 58 | 18.759 | 37.503 | −24.760 | 1.00 | 24.19 | N |
| ATOM | 3805 | C | ASN | I | 58 | 14.788 | 38.737 | −23.672 | 1.00 | 26.27 | C |
| ATOM | 3806 | O | ASN | I | 58 | 14.082 | 38.456 | −22.699 | 1.00 | 26.30 | O |
| ATOM | 3807 | N | TYR | I | 59 | 14.442 | 38.469 | −24.925 | 1.00 | 27.05 | N |
| ATOM | 3808 | CA | TYR | I | 59 | 13.124 | 37.948 | −25.268 | 1.00 | 27.55 | C |
| ATOM | 3809 | CB | TYR | I | 59 | 12.394 | 38.948 | −26.150 | 1.00 | 27.32 | C |
| ATOM | 3810 | CG | TYR | I | 59 | 11.909 | 40.197 | −25.467 | 1.00 | 26.32 | C |
| ATOM | 3811 | CD1 | TYR | I | 59 | 12.743 | 41.306 | −25.305 | 1.00 | 25.02 | C |
| ATOM | 3812 | CE1 | TYR | I | 59 | 12.267 | 42.484 | −24.684 | 1.00 | 25.32 | C |
| ATOM | 3813 | CZ | TYR | I | 59 | 10.946 | 42.533 | −24.237 | 1.00 | 25.87 | C |
| ATOM | 3814 | OH | TYR | I | 59 | 10.440 | 43.661 | −23.617 | 1.00 | 25.62 | O |
| ATOM | 3815 | CE2 | TYR | I | 59 | 10.112 | 41.440 | −24.409 | 1.00 | 25.45 | C |
| ATOM | 3816 | CD2 | TYR | I | 59 | 10.594 | 40.288 | −25.020 | 1.00 | 25.33 | C |
| ATOM | 3817 | C | TYR | I | 59 | 13.198 | 36.640 | −26.032 | 1.00 | 28.35 | C |
| ATOM | 3818 | O | TYR | I | 59 | 14.132 | 36.419 | −26.803 | 1.00 | 27.91 | O |
| ATOM | 3819 | N | ASN | I | 60 | 12.201 | 35.781 | −25.807 | 1.00 | 29.49 | N |
| ATOM | 3820 | CA | ASN | I | 60 | 11.884 | 34.683 | −26.716 | 1.00 | 30.50 | C |
| ATOM | 3821 | CB | ASN | I | 60 | 10.774 | 33.816 | −26.111 | 1.00 | 30.53 | C |
| ATOM | 3822 | CG | ASN | I | 60 | 10.364 | 32.620 | −26.997 | 1.00 | 30.52 | C |
| ATOM | 3823 | OD1 | ASN | I | 60 | 10.616 | 32.581 | −28.209 | 1.00 | 30.50 | O |
| ATOM | 3824 | ND2 | ASN | I | 60 | 9.708 | 31.640 | −26.373 | 1.00 | 28.16 | N |
| ATOM | 3825 | C | ASN | I | 60 | 11.390 | 35.355 | −27.995 | 1.00 | 31.51 | C |
| ATOM | 3826 | O | ASN | I | 60 | 10.499 | 36.222 | −27.945 | 1.00 | 31.15 | O |
| ATOM | 3827 | N | GLU | I | 61 | 11.986 | 34.962 | −29.125 | 1.00 | 32.74 | N |
| ATOM | 3828 | CA | GLU | I | 61 | 11.704 | 35.554 | −30.441 | 1.00 | 34.03 | C |
| ATOM | 3829 | CB | GLU | I | 61 | 12.486 | 34.808 | −31.531 | 1.00 | 34.61 | C |
| ATOM | 3830 | CG | GLU | I | 61 | 13.023 | 35.707 | −32.664 | 1.00 | 37.77 | C |
| ATOM | 3831 | CD | GLU | I | 61 | 13.099 | 34.973 | −33.998 | 1.00 | 42.64 | C |
| ATOM | 3832 | OE1 | GLU | I | 61 | 13.103 | 33.720 | −33.982 | 1.00 | 44.75 | O |
| ATOM | 3833 | OE2 | GLU | I | 61 | 13.146 | 35.636 | −35.064 | 1.00 | 43.76 | O |
| ATOM | 3834 | C | GLU | I | 61 | 10.211 | 35.553 | −30.776 | 1.00 | 33.92 | C |
| ATOM | 3835 | O | GLU | I | 61 | 9.713 | 36.477 | −31.408 | 1.00 | 33.81 | O |
| ATOM | 3836 | N | ASN | I | 62 | 9.504 | 34.519 | −30.324 | 1.00 | 34.20 | N |
| ATOM | 3837 | CA | ASN | I | 62 | 8.085 | 34.349 | −30.613 | 1.00 | 34.55 | C |
| ATOM | 3838 | CB | ASN | I | 62 | 7.634 | 32.918 | −30.270 | 1.00 | 35.08 | C |
| ATOM | 3839 | CG | ASN | I | 62 | 8.362 | 31.850 | −31.100 | 1.00 | 36.67 | C |
| ATOM | 3840 | OD1 | ASN | I | 62 | 8.455 | 31.950 | −32.327 | 1.00 | 39.13 | O |
| ATOM | 3841 | ND2 | ASN | I | 62 | 8.879 | 30.826 | −30.427 | 1.00 | 37.78 | N |
| ATOM | 3842 | C | ASN | I | 62 | 7.206 | 35.365 | −29.902 | 1.00 | 34.22 | C |
| ATOM | 3843 | O | ASN | I | 62 | 5.999 | 35.399 | −30.117 | 1.00 | 34.27 | O |
| ATOM | 3844 | N | PHE | I | 63 | 7.811 | 36.207 | −29.073 | 1.00 | 33.98 | N |
| ATOM | 3845 | CA | PHE | I | 63 | 7.040 | 37.143 | −28.274 | 1.00 | 34.07 | C |
| ATOM | 3846 | CB | PHE | I | 63 | 7.235 | 36.875 | −26.778 | 1.00 | 33.90 | C |
| ATOM | 3847 | CG | PHE | I | 63 | 6.542 | 35.647 | −26.291 | 1.00 | 32.74 | C |
| ATOM | 3848 | CD1 | PHE | I | 63 | 7.261 | 34.494 | −26.023 | 1.00 | 31.03 | C |
| ATOM | 3849 | CE1 | PHE | I | 63 | 6.622 | 33.355 | −25.578 | 1.00 | 31.00 | C |
| ATOM | 3850 | CZ | PHE | I | 63 | 5.252 | 33.353 | −25.394 | 1.00 | 30.44 | C |
| ATOM | 3851 | CE2 | PHE | I | 63 | 4.523 | 34.496 | −25.651 | 1.00 | 31.72 | C |
| ATOM | 3852 | CD2 | PHE | I | 63 | 5.168 | 35.640 | −26.095 | 1.00 | 32.22 | C |
| ATOM | 3853 | C | PHE | I | 63 | 7.346 | 38.596 | −28.567 | 1.00 | 34.49 | C |
| ATOM | 3854 | O | PHE | I | 63 | 6.650 | 39.480 | −28.056 | 1.00 | 34.21 | O |
| ATOM | 3855 | N | LYS | I | 64 | 8.374 | 38.853 | −29.375 | 1.00 | 35.33 | N |
| ATOM | 3856 | CA | LYS | I | 64 | 8.764 | 40.240 | −29.640 | 1.00 | 36.28 | C |
| ATOM | 3857 | CB | LYS | I | 64 | 10.104 | 40.378 | −30.376 | 1.00 | 36.44 | C |
| ATOM | 3858 | CG | LYS | I | 64 | 10.495 | 41.879 | −30.625 | 1.00 | 38.36 | C |
| ATOM | 3859 | CD | LYS | I | 64 | 12.003 | 42.152 | −30.608 | 1.00 | 40.21 | C |
| ATOM | 3860 | CE | LYS | I | 64 | 12.487 | 42.520 | −29.200 | 1.00 | 41.83 | C |
| ATOM | 3861 | NZ | LYS | I | 64 | 13.941 | 42.901 | −29.177 | 1.00 | 41.46 | N |
| ATOM | 3862 | C | LYS | I | 64 | 7.655 | 40.992 | −30.362 | 1.00 | 36.48 | C |
| ATOM | 3863 | O | LYS | I | 64 | 7.025 | 40.474 | −31.299 | 1.00 | 36.28 | O |
| ATOM | 3864 | N | GLY | I | 65 | 7.403 | 42.206 | −29.881 | 1.00 | 36.69 | N |
| ATOM | 3865 | CA | GLY | I | 65 | 6.359 | 43.036 | −30.445 | 1.00 | 37.17 | C |
| ATOM | 3866 | C | GLY | I | 65 | 4.976 | 42.582 | −30.034 | 1.00 | 37.05 | C |
| ATOM | 3867 | O | GLY | I | 65 | 3.986 | 43.177 | −30.438 | 1.00 | 37.18 | O |
| ATOM | 3868 | N | GLN | I | 66 | 4.912 | 41.513 | −29.251 | 1.00 | 37.04 | N |
| ATOM | 3869 | CA | GLN | I | 66 | 3.694 | 41.155 | −28.552 | 1.00 | 37.30 | C |
| ATOM | 3870 | CB | GLN | I | 66 | 3.522 | 39.635 | −28.489 | 1.00 | 37.82 | C |
| ATOM | 3871 | CG | GLN | I | 66 | 2.981 | 39.008 | −29.751 | 1.00 | 39.96 | C |
| ATOM | 3872 | CD | GLN | I | 66 | 1.610 | 39.555 | −30.148 | 1.00 | 43.50 | C |
| ATOM | 3873 | OE1 | GLN | I | 66 | 0.673 | 39.624 | −29.319 | 1.00 | 43.90 | O |
| ATOM | 3874 | NE2 | GLN | I | 66 | 1.483 | 39.947 | −31.427 | 1.00 | 42.73 | N |
| ATOM | 3875 | C | GLN | I | 66 | 3.772 | 41.709 | −27.142 | 1.00 | 36.72 | C |
| ATOM | 3876 | O | GLN | I | 66 | 2.893 | 42.442 | −26.703 | 1.00 | 37.05 | O |
| ATOM | 3877 | N | ALA | I | 67 | 4.839 | 41.352 | −26.438 | 1.00 | 35.76 | N |
| ATOM | 3878 | CA | ALA | I | 67 | 4.958 | 41.677 | −25.033 | 1.00 | 35.05 | C |
| ATOM | 3879 | CB | ALA | I | 67 | 5.008 | 40.396 | −24.193 | 1.00 | 35.21 | C |
| ATOM | 3880 | C | ALA | I | 67 | 6.187 | 42.518 | −24.786 | 1.00 | 34.46 | C |
| ATOM | 3881 | O | ALA | I | 67 | 7.196 | 42.363 | −25.470 | 1.00 | 34.59 | O |
| ATOM | 3882 | N | THR | I | 68 | 6.089 | 43.398 | −23.794 | 1.00 | 33.42 | N |
| ATOM | 3883 | CA | THR | I | 68 | 7.174 | 44.274 | −23.416 | 1.00 | 32.57 | C |
| ATOM | 3884 | CB | THR | I | 68 | 6.842 | 45.728 | −23.801 | 1.00 | 32.85 | C |
| ATOM | 3885 | OG1 | THR | I | 68 | 6.428 | 45.771 | −25.172 | 1.00 | 33.56 | O |
| ATOM | 3886 | CG2 | THR | I | 68 | 8.045 | 46.649 | −23.591 | 1.00 | 32.47 | C |
| ATOM | 3887 | C | THR | I | 68 | 7.397 | 44.181 | −21.907 | 1.00 | 31.92 | C |
| ATOM | 3888 | O | THR | I | 68 | 6.464 | 44.390 | −21.119 | 1.00 | 31.33 | O |
| ATOM | 3889 | N | LEU | I | 69 | 8.632 | 43.866 | −21.514 | 1.00 | 31.00 | N |
| ATOM | 3890 | CA | LEU | I | 69 | 9.030 | 43.897 | −20.100 | 1.00 | 30.19 | C |
| ATOM | 3891 | CB | LEU | I | 69 | 10.138 | 42.878 | −19.797 | 1.00 | 29.91 | C |
| ATOM | 3892 | CG | LEU | I | 69 | 9.942 | 41.408 | −20.154 | 1.00 | 29.78 | C |
| ATOM | 3893 | CD1 | LEU | I | 69 | 11.137 | 40.564 | −19.739 | 1.00 | 29.24 | C |
| ATOM | 3894 | CD2 | LEU | I | 69 | 8.689 | 40.901 | −19.513 | 1.00 | 31.17 | C |
| ATOM | 3895 | C | LEU | I | 69 | 9.513 | 45.285 | −19.703 | 1.00 | 29.81 | C |
| ATOM | 3896 | O | LEU | I | 69 | 10.213 | 45.957 | −20.462 | 1.00 | 30.21 | O |
| ATOM | 3897 | N | SER | I | 70 | 9.141 | 45.714 | −18.509 | 1.00 | 29.16 | N |
| ATOM | 3898 | CA | SER | I | 70 | 9.601 | 46.990 | −17.988 | 1.00 | 29.03 | C |
| ATOM | 3899 | CB | SER | I | 70 | 8.656 | 48.126 | −18.412 | 1.00 | 28.94 | C |
| ATOM | 3900 | OG | SER | I | 70 | 7.351 | 47.961 | −17.886 | 1.00 | 27.48 | O |
| ATOM | 3901 | C | SER | I | 70 | 9.689 | 46.869 | −16.470 | 1.00 | 29.23 | C |
| ATOM | 3902 | O | SER | I | 70 | 9.298 | 45.833 | −15.908 | 1.00 | 29.28 | O |
| ATOM | 3903 | N | ALA | I | 71 | 10.214 | 47.898 | −15.811 | 1.00 | 28.89 | N |
| ATOM | 3904 | CA | ALA | I | 71 | 10.341 | 47.877 | −14.359 | 1.00 | 29.11 | C |
| ATOM | 3905 | CB | ALA | I | 71 | 11.496 | 46.979 | −13.924 | 1.00 | 28.92 | C |
| ATOM | 3906 | C | ALA | I | 71 | 10.486 | 49.259 | −13.761 | 1.00 | 29.35 | C |
| ATOM | 3907 | O | ALA | I | 71 | 11.066 | 50.161 | −14.367 | 1.00 | 29.02 | O |
| ATOM | 3908 | N | ASP | I | 72 | 9.950 | 49.395 | −12.555 | 1.00 | 30.26 | N |
| ATOM | 3909 | CA | ASP | I | 72 | 9.909 | 50.645 | −11.823 | 1.00 | 31.24 | C |
| ATOM | 3910 | CB | ASP | I | 72 | 8.456 | 51.004 | −11.544 | 1.00 | 31.44 | C |
| ATOM | 3911 | CG | ASP | I | 72 | 8.293 | 52.341 | −10.847 | 1.00 | 33.41 | C |
| ATOM | 3912 | OD1 | ASP | I | 72 | 9.234 | 52.819 | −10.172 | 1.00 | 35.01 | O |
| ATOM | 3913 | OD2 | ASP | I | 72 | 7.189 | 52.915 | −10.967 | 1.00 | 35.97 | O |
| ATOM | 3914 | C | ASP | I | 72 | 10.638 | 50.454 | −10.514 | 1.00 | 31.59 | C |
| ATOM | 3915 | O | ASP | I | 72 | 10.077 | 49.913 | −9.573 | 1.00 | 31.92 | O |
| ATOM | 3916 | N | LYS | I | 73 | 11.874 | 50.931 | −10.439 | 1.00 | 32.38 | N |
| ATOM | 3917 | CA | LYS | I | 73 | 12.700 | 50.707 | −9.253 | 1.00 | 33.12 | C |
| ATOM | 3918 | CB | LYS | I | 73 | 14.189 | 50.961 | −9.549 | 1.00 | 33.65 | C |
| ATOM | 3919 | CG | LYS | I | 73 | 14.644 | 52.424 | −9.526 | 1.00 | 35.91 | C |
| ATOM | 3920 | CD | LYS | I | 73 | 16.117 | 52.526 | −9.981 | 1.00 | 39.14 | C |
| ATOM | 3921 | CE | LYS | I | 73 | 16.634 | 53.966 | −10.004 | 1.00 | 40.91 | C |
| ATOM | 3922 | NZ | LYS | I | 73 | 16.821 | 54.549 | −8.635 | 1.00 | 41.55 | N |
| ATOM | 3923 | C | LYS | I | 73 | 12.232 | 51.421 | −7.972 | 1.00 | 32.85 | C |
| ATOM | 3924 | O | LYS | I | 73 | 12.578 | 50.992 | −6.860 | 1.00 | 33.16 | O |
| ATOM | 3925 | N | SER | I | 74 | 11.436 | 52.480 | −8.108 | 1.00 | 32.20 | N |
| ATOM | 3926 | CA | SER | I | 74 | 10.959 | 53.201 | −6.916 | 1.00 | 31.80 | C |
| ATOM | 3927 | CB | SER | I | 74 | 10.448 | 54.606 | −7.267 | 1.00 | 32.03 | C |
| ATOM | 3928 | OG | SER | I | 74 | 9.368 | 54.561 | −8.187 | 1.00 | 33.89 | O |
| ATOM | 3929 | C | SER | I | 74 | 9.921 | 52.420 | −6.109 | 1.00 | 31.03 | C |
| ATOM | 3930 | O | SER | I | 74 | 9.867 | 52.529 | −4.885 | 1.00 | 31.13 | O |
| ATOM | 3931 | N | SER | I | 75 | 9.111 | 51.618 | −6.794 | 1.00 | 30.16 | N |
| ATOM | 3932 | CA | SER | I | 75 | 8.147 | 50.743 | −6.125 | 1.00 | 29.20 | C |
| ATOM | 3933 | CB | SER | I | 75 | 6.766 | 50.876 | −6.778 | 1.00 | 29.38 | C |
| ATOM | 3934 | OG | SER | I | 75 | 6.806 | 50.517 | −8.156 | 1.00 | 29.97 | O |
| ATOM | 3935 | C | SER | I | 75 | 8.595 | 49.266 | −6.103 | 1.00 | 28.47 | C |
| ATOM | 3936 | O | SER | I | 75 | 7.911 | 48.410 | −5.535 | 1.00 | 28.65 | O |
| ATOM | 3937 | N | SER | I | 76 | 9.740 | 48.974 | −6.716 | 1.00 | 27.05 | N |
| ATOM | 3938 | CA | SER | I | 76 | 10.218 | 47.599 | −6.858 | 1.00 | 26.15 | C |
| ATOM | 3939 | CB | SER | I | 76 | 10.569 | 46.990 | −5.496 | 1.00 | 26.06 | C |
| ATOM | 3940 | OG | SER | I | 76 | 11.602 | 47.715 | −4.868 | 1.00 | 26.09 | O |
| ATOM | 3941 | C | SER | I | 76 | 9.218 | 46.694 | −7.596 | 1.00 | 25.41 | C |
| ATOM | 3942 | O | SER | I | 76 | 9.049 | 45.530 | −7.234 | 1.00 | 25.32 | O |
| ATOM | 3943 | N | THR | I | 77 | 8.572 | 47.234 | −8.628 | 1.00 | 24.21 | N |
| ATOM | 3944 | CA | THR | I | 77 | 7.609 | 46.479 | −9.413 | 1.00 | 23.41 | C |
| ATOM | 3945 | CB | THR | I | 77 | 6.239 | 47.199 | −9.497 | 1.00 | 23.31 | C |
| ATOM | 3946 | OG1 | THR | I | 77 | 5.791 | 47.544 | −8.180 | 1.00 | 24.10 | O |
| ATOM | 3947 | CG2 | THR | I | 77 | 5.203 | 46.307 | −10.127 | 1.00 | 21.91 | C |
| ATOM | 3948 | C | THR | I | 77 | 8.125 | 46.195 | −10.815 | 1.00 | 22.96 | C |
| ATOM | 3949 | O | THR | I | 77 | 8.679 | 47.073 | −11.477 | 1.00 | 22.89 | O |
| ATOM | 3950 | N | ALA | I | 78 | 7.937 | 44.955 | −11.249 | 1.00 | 22.40 | N |
| ATOM | 3951 | CA | ALA | I | 78 | 8.213 | 44.543 | −12.615 | 1.00 | 22.17 | C |
| ATOM | 3952 | CB | ALA | I | 78 | 8.979 | 43.237 | −12.638 | 1.00 | 21.55 | C |
| ATOM | 3953 | C | ALA | I | 78 | 6.890 | 44.396 | −13.362 | 1.00 | 22.20 | C |
| ATOM | 3954 | O | ALA | I | 78 | 5.887 | 43.942 | −12.791 | 1.00 | 22.21 | O |
| ATOM | 3955 | N | TYR | I | 79 | 6.893 | 44.779 | −14.635 | 1.00 | 22.05 | N |
| ATOM | 3956 | CA | TYR | I | 79 | 5.673 | 44.786 | −15.432 | 1.00 | 22.35 | C |
| ATOM | 3957 | CB | TYR | I | 79 | 5.283 | 46.210 | −15.818 | 1.00 | 21.82 | C |
| ATOM | 3958 | CG | TYR | I | 79 | 5.025 | 47.133 | −14.654 | 1.00 | 20.07 | C |
| ATOM | 3959 | CD1 | TYR | I | 79 | 3.771 | 47.180 | −14.053 | 1.00 | 19.19 | C |
| ATOM | 3960 | CE1 | TYR | I | 79 | 3.500 | 48.035 | −12.992 | 1.00 | 17.61 | C |
| ATOM | 3961 | CZ | TYR | I | 79 | 4.489 | 48.865 | −12.515 | 1.00 | 18.06 | C |
| ATOM | 3962 | OH | TYR | I | 79 | 4.201 | 49.688 | −11.448 | 1.00 | 18.23 | O |
| ATOM | 3963 | CE2 | TYR | I | 79 | 5.762 | 48.846 | −13.090 | 1.00 | 19.12 | C |
| ATOM | 3964 | CD2 | TYR | I | 79 | 6.021 | 47.979 | −14.170 | 1.00 | 18.72 | C |
| ATOM | 3965 | C | TYR | I | 79 | 5.780 | 43.944 | −16.689 | 1.00 | 23.16 | C |
| ATOM | 3966 | O | TYR | I | 79 | 6.866 | 43.687 | −17.205 | 1.00 | 23.45 | O |
| ATOM | 3967 | N | LEU | I | 80 | 4.629 | 43.511 | −17.169 | 1.00 | 24.20 | N |
| ATOM | 3968 | CA | LEU | I | 80 | 4.526 | 42.861 | −18.456 | 1.00 | 25.36 | C |
| ATOM | 3969 | CB | LEU | I | 80 | 4.333 | 41.357 | −18.290 | 1.00 | 25.02 | C |
| ATOM | 3970 | CG | LEU | I | 80 | 4.083 | 40.463 | −19.503 | 1.00 | 25.47 | C |
| ATOM | 3971 | CD1 | LEU | I | 80 | 5.281 | 40.371 | −20.466 | 1.00 | 24.95 | C |
| ATOM | 3972 | CD2 | LEU | I | 80 | 3.708 | 39.094 | −19.011 | 1.00 | 25.03 | C |
| ATOM | 3973 | C | LEU | I | 80 | 3.337 | 43.520 | −19.132 | 1.00 | 26.86 | C |
| ATOM | 3974 | O | LEU | I | 80 | 2.201 | 43.500 | −18.616 | 1.00 | 26.81 | O |
| ATOM | 3975 | N | GLN | I | 81 | 3.642 | 44.147 | −20.264 | 1.00 | 28.60 | N |
| ATOM | 3976 | CA | GLN | I | 81 | 2.728 | 44.986 | −21.019 | 1.00 | 30.31 | C |
| ATOM | 3977 | CB | GLN | I | 81 | 3.457 | 46.263 | −21.424 | 1.00 | 30.67 | C |
| ATOM | 3978 | CG | GLN | I | 81 | 2.626 | 47.513 | −21.592 | 1.00 | 33.74 | C |
| ATOM | 3979 | CD | GLN | I | 81 | 3.428 | 48.785 | −21.244 | 1.00 | 38.01 | C |
| ATOM | 3980 | OE1 | GLN | I | 81 | 2.878 | 49.739 | −20.679 | 1.00 | 40.13 | O |
| ATOM | 3981 | NE2 | GLN | I | 81 | 4.734 | 48.793 | −21.564 | 1.00 | 37.71 | N |
| ATOM | 3982 | C | GLN | I | 81 | 2.373 | 44.216 | −22.268 | 1.00 | 31.05 | C |
| ATOM | 3983 | O | GLN | I | 81 | 3.221 | 43.558 | −22.878 | 1.00 | 30.86 | O |
| ATOM | 3984 | N | TRP | I | 82 | 1.117 | 44.311 | −22.664 | 1.00 | 32.34 | N |
| ATOM | 3985 | CA | TRP | I | 82 | 0.648 | 43.586 | −23.813 | 1.00 | 33.59 | C |
| ATOM | 3986 | CB | TRP | I | 82 | −0.121 | 42.353 | −23.354 | 1.00 | 34.09 | C |
| ATOM | 3987 | CG | TRP | I | 82 | 0.219 | 41.177 | −24.142 | 1.00 | 35.84 | C |
| ATOM | 3988 | CD1 | TRP | I | 82 | 0.037 | 41.018 | −25.472 | 1.00 | 38.42 | C |
| ATOM | 3989 | NE1 | TRP | I | 82 | 0.484 | 39.785 | −25.867 | 1.00 | 39.58 | N |
| ATOM | 3990 | CE2 | TRP | I | 82 | 0.974 | 39.125 | −24.776 | 1.00 | 38.64 | C |
| ATOM | 3991 | CD2 | TRP | I | 82 | 0.820 | 39.979 | −23.666 | 1.00 | 37.32 | C |
| ATOM | 3992 | CE3 | TRP | I | 82 | 1.240 | 39.540 | −22.409 | 1.00 | 39.39 | C |
| ATOM | 3993 | CZ3 | TRP | I | 82 | 1.804 | 38.279 | −22.302 | 1.00 | 40.63 | C |
| ATOM | 3994 | CH2 | TRP | I | 82 | 1.950 | 37.445 | −23.434 | 1.00 | 41.69 | C |
| ATOM | 3995 | CZ2 | TRP | I | 82 | 1.538 | 37.851 | −24.675 | 1.00 | 40.40 | C |
| ATOM | 3996 | C | TRP | I | 82 | −0.262 | 44.485 | −24.611 | 1.00 | 34.18 | C |
| ATOM | 3997 | O | TRP | I | 82 | −1.427 | 44.662 | −24.258 | 1.00 | 34.70 | O |
| ATOM | 3998 | N | SER | I | 82A | 0.267 | 45.090 | −25.666 | 1.00 | 34.63 | N |
| ATOM | 3999 | CA | SER | I | 82A | −0.594 | 45.767 | −26.625 | 1.00 | 35.00 | C |
| ATOM | 4000 | CB | SER | I | 82A | 0.175 | 46.832 | −27.406 | 1.00 | 35.13 | C |
| ATOM | 4001 | OG | SER | I | 82A | 1.240 | 46.253 | −28.137 | 1.00 | 36.25 | O |
| ATOM | 4002 | C | SER | I | 82A | −1.188 | 44.709 | −27.560 | 1.00 | 34.97 | C |
| ATOM | 4003 | O | SER | I | 82A | −0.487 | 43.776 | −27.998 | 1.00 | 35.26 | O |
| ATOM | 4004 | N | SER | I | 82B | −2.485 | 44.850 | −27.827 | 1.00 | 34.46 | N |
| ATOM | 4005 | CA | SER | I | 82B | −3.242 | 43.944 | −28.709 | 1.00 | 33.86 | C |
| ATOM | 4006 | CB | SER | I | 82B | −2.896 | 44.163 | −30.191 | 1.00 | 33.87 | C |
| ATOM | 4007 | OG | SER | I | 82B | −3.930 | 44.893 | −30.835 | 1.00 | 34.53 | O |
| ATOM | 4008 | C | SER | I | 82B | −3.220 | 42.460 | −28.331 | 1.00 | 33.07 | C |
| ATOM | 4009 | O | SER | I | 82B | −2.498 | 41.658 | −28.924 | 1.00 | 32.79 | O |
| ATOM | 4010 | N | LEU | I | 82C | −4.050 | 42.113 | −27.348 | 1.00 | 32.52 | N |
| ATOM | 4011 | CA | LEU | I | 82C | −4.215 | 40.732 | −26.922 | 1.00 | 31.60 | C |
| ATOM | 4012 | CB | LEU | I | 82C | −4.991 | 40.651 | −25.610 | 1.00 | 30.92 | C |
| ATOM | 4013 | CG | LEU | I | 82C | −4.284 | 41.119 | −24.331 | 1.00 | 28.99 | C |
| ATOM | 4014 | CD1 | LEU | I | 82C | −5.301 | 41.612 | −23.318 | 1.00 | 26.49 | C |
| ATOM | 4015 | CD2 | LEU | I | 82C | −3.364 | 40.063 | −23.722 | 1.00 | 24.61 | C |
| ATOM | 4016 | C | LEU | I | 82C | −4.943 | 39.953 | −27.991 | 1.00 | 31.88 | C |
| ATOM | 4017 | O | LEU | I | 82C | −5.831 | 40.483 | −28.660 | 1.00 | 31.82 | O |
| ATOM | 4018 | N | LYS | I | 83 | −4.544 | 38.700 | −28.167 | 1.00 | 32.15 | N |
| ATOM | 4019 | CA | LYS | I | 83 | −5.299 | 37.763 | −28.991 | 1.00 | 32.51 | C |
| ATOM | 4020 | CB | LYS | I | 83 | −4.495 | 37.315 | −30.211 | 1.00 | 32.54 | C |
| ATOM | 4021 | CG | LYS | I | 83 | −3.126 | 36.729 | −29.914 | 1.00 | 35.20 | C |
| ATOM | 4022 | CD | LYS | I | 83 | −2.331 | 36.583 | −31.199 | 1.00 | 38.11 | C |
| ATOM | 4023 | CE | LYS | I | 83 | −1.813 | 35.166 | −31.363 | 1.00 | 39.86 | C |
| ATOM | 4024 | NZ | LYS | I | 83 | −1.984 | 34.710 | −32.777 | 1.00 | 41.50 | N |
| ATOM | 4025 | C | LYS | I | 83 | −5.695 | 36.603 | −28.103 | 1.00 | 32.23 | C |
| ATOM | 4026 | O | LYS | I | 83 | −5.122 | 36.445 | −27.033 | 1.00 | 32.58 | O |
| ATOM | 4027 | N | ALA | I | 84 | −6.674 | 35.811 | −28.534 | 1.00 | 32.14 | N |
| ATOM | 4028 | CA | ALA | I | 84 | −7.297 | 34.769 | −27.686 | 1.00 | 32.07 | C |
| ATOM | 4029 | CB | ALA | I | 84 | −8.450 | 34.074 | −28.435 | 1.00 | 31.83 | C |
| ATOM | 4030 | C | ALA | I | 84 | −6.319 | 33.727 | −27.148 | 1.00 | 31.98 | C |
| ATOM | 4031 | O | ALA | I | 84 | −6.490 | 33.220 | −26.039 | 1.00 | 31.95 | O |
| ATOM | 4032 | N | SER | I | 85 | −5.302 | 33.418 | −27.948 | 1.00 | 31.99 | N |
| ATOM | 4033 | CA | SER | I | 85 | −4.327 | 32.392 | −27.612 | 1.00 | 32.07 | C |
| ATOM | 4034 | CB | SER | I | 85 | −3.559 | 31.945 | −28.867 | 1.00 | 31.82 | C |
| ATOM | 4035 | OG | SER | I | 85 | −2.537 | 32.879 | −29.185 | 1.00 | 32.30 | O |
| ATOM | 4036 | C | SER | I | 85 | −3.360 | 32.854 | −26.503 | 1.00 | 31.74 | C |
| ATOM | 4037 | O | SER | I | 85 | −2.561 | 32.057 | −26.006 | 1.00 | 32.35 | O |
| ATOM | 4038 | N | ASP | I | 86 | −3.437 | 34.134 | −26.138 | 1.00 | 31.03 | N |
| ATOM | 4039 | CA | ASP | I | 86 | −2.724 | 34.685 | −24.980 | 1.00 | 30.13 | C |
| ATOM | 4040 | CB | ASP | I | 86 | −2.705 | 36.215 | −25.051 | 1.00 | 30.32 | C |
| ATOM | 4041 | CG | ASP | I | 86 | −1.775 | 36.739 | −26.123 | 1.00 | 31.93 | C |
| ATOM | 4042 | OD1 | ASP | I | 86 | −1.921 | 37.928 | −26.501 | 1.00 | 33.33 | O |
| ATOM | 4043 | OD2 | ASP | I | 86 | −0.893 | 35.969 | −26.580 | 1.00 | 33.09 | O |
| ATOM | 4044 | C | ASP | I | 86 | −3.333 | 34.234 | −23.636 | 1.00 | 29.01 | C |
| ATOM | 4045 | O | ASP | I | 86 | −2.770 | 34.508 | −22.570 | 1.00 | 28.25 | O |
| ATOM | 4046 | N | THR | I | 87 | −4.489 | 33.572 | −23.699 | 1.00 | 27.73 | N |
| ATOM | 4047 | CA | THR | I | 87 | −5.109 | 32.985 | −22.524 | 1.00 | 27.02 | C |
| ATOM | 4048 | CB | THR | I | 87 | −6.372 | 32.189 | −22.890 | 1.00 | 27.06 | C |
| ATOM | 4049 | OG1 | THR | I | 87 | −7.338 | 33.067 | −23.466 | 1.00 | 27.27 | O |
| ATOM | 4050 | CG2 | THR | I | 87 | −6.978 | 31.523 | −21.654 | 1.00 | 27.03 | C |
| ATOM | 4051 | C | THR | I | 87 | −4.100 | 32.031 | −21.904 | 1.00 | 26.68 | C |
| ATOM | 4052 | O | THR | I | 87 | −3.691 | 31.053 | −22.559 | 1.00 | 26.99 | O |
| ATOM | 4053 | N | ALA | I | 88 | −3.693 | 32.321 | −20.662 | 1.00 | 25.20 | N |
| ATOM | 4054 | CA | ALA | I | 88 | −2.677 | 31.528 | −19.965 | 1.00 | 23.84 | C |
| ATOM | 4055 | CB | ALA | I | 88 | −1.365 | 31.555 | −20.733 | 1.00 | 23.47 | C |
| ATOM | 4056 | C | ALA | I | 88 | −2.444 | 32.002 | −18.540 | 1.00 | 23.13 | C |
| ATOM | 4057 | O | ALA | I | 88 | −2.875 | 33.086 | −18.160 | 1.00 | 23.02 | O |
| ATOM | 4058 | N | MET | I | 89 | −1.758 | 31.170 | −17.760 | 1.00 | 22.54 | N |
| ATOM | 4059 | CA | MET | I | 89 | −1.212 | 31.584 | −16.472 | 1.00 | 22.20 | C |
| ATOM | 4060 | CB | MET | I | 89 | −1.066 | 30.399 | −15.516 | 1.00 | 21.98 | C |
| ATOM | 4061 | CG | MET | I | 89 | −0.555 | 30.801 | −14.134 | 1.00 | 22.40 | C |
| ATOM | 4062 | SD | MET | I | 89 | −1.691 | 31.856 | −13.201 | 1.00 | 24.29 | S |
| ATOM | 4063 | CE | MET | I | 89 | −2.917 | 30.637 | −12.718 | 1.00 | 24.23 | C |
| ATOM | 4064 | C | MET | I | 89 | 0.141 | 32.256 | −16.680 | 1.00 | 21.69 | C |
| ATOM | 4065 | O | MET | I | 89 | 1.027 | 31.709 | −17.349 | 1.00 | 21.66 | O |
| ATOM | 4066 | N | TYR | I | 90 | 0.293 | 33.440 | −16.104 | 1.00 | 21.08 | N |
| ATOM | 4067 | CA | TYR | I | 90 | 1.530 | 34.200 | −16.236 | 1.00 | 20.41 | C |
| ATOM | 4068 | CB | TYR | I | 90 | 1.232 | 35.585 | −16.807 | 1.00 | 20.37 | C |
| ATOM | 4069 | CG | TYR | I | 90 | 0.834 | 35.523 | −18.244 | 1.00 | 20.29 | C |
| ATOM | 4070 | CD1 | TYR | I | 90 | −0.484 | 35.263 | −18.611 | 1.00 | 20.59 | C |
| ATOM | 4071 | CE1 | TYR | I | 90 | −0.859 | 35.169 | −19.957 | 1.00 | 20.41 | C |
| ATOM | 4072 | CZ | TYR | I | 90 | 0.097 | 35.326 | −20.952 | 1.00 | 20.07 | C |
| ATOM | 4073 | OH | TYR | I | 90 | −0.267 | 35.244 | −22.280 | 1.00 | 19.27 | O |
| ATOM | 4074 | CE2 | TYR | I | 90 | 1.419 | 35.573 | −20.608 | 1.00 | 21.40 | C |
| ATOM | 4075 | CD2 | TYR | I | 90 | 1.782 | 35.671 | −19.251 | 1.00 | 21.96 | C |
| ATOM | 4076 | C | TYR | I | 90 | 2.261 | 34.299 | −14.899 | 1.00 | 20.32 | C |
| ATOM | 4077 | O | TYR | I | 90 | 1.739 | 34.886 | −13.937 | 1.00 | 20.17 | O |
| ATOM | 4078 | N | PHE | I | 91 | 3.451 | 33.699 | −14.845 | 1.00 | 19.82 | N |
| ATOM | 4079 | CA | PHE | I | 91 | 4.305 | 33.728 | −13.663 | 1.00 | 19.70 | C |
| ATOM | 4080 | CB | PHE | I | 91 | 4.904 | 32.336 | −13.402 | 1.00 | 19.87 | C |
| ATOM | 4081 | CG | PHE | I | 91 | 3.914 | 31.304 | −12.887 | 1.00 | 20.31 | C |
| ATOM | 4082 | CD1 | PHE | I | 91 | 3.461 | 31.339 | −11.578 | 1.00 | 19.79 | C |
| ATOM | 4083 | CE1 | PHE | I | 91 | 2.561 | 30.374 | −11.100 | 1.00 | 19.49 | C |
| ATOM | 4084 | CZ | PHE | I | 91 | 2.130 | 29.360 | −11.931 | 1.00 | 18.65 | C |
| ATOM | 4085 | CE2 | PHE | I | 91 | 2.592 | 29.296 | −13.225 | 1.00 | 19.31 | C |
| ATOM | 4086 | CD2 | PHE | I | 91 | 3.485 | 30.266 | −13.704 | 1.00 | 20.10 | C |
| ATOM | 4087 | C | PHE | I | 91 | 5.468 | 34.708 | −13.835 | 1.00 | 19.71 | C |
| ATOM | 4088 | O | PHE | I | 91 | 6.057 | 34.805 | −14.928 | 1.00 | 19.56 | O |
| ATOM | 4089 | N | CYS | I | 92 | 5.825 | 35.407 | −12.759 | 1.00 | 19.62 | N |
| ATOM | 4090 | CA | CYS | I | 92 | 7.149 | 36.052 | −12.681 | 1.00 | 19.59 | C |
| ATOM | 4091 | CB | CYS | I | 92 | 7.055 | 37.509 | −12.229 | 1.00 | 19.35 | C |
| ATOM | 4092 | SG | CYS | I | 92 | 6.396 | 37.709 | −10.580 | 1.00 | 21.55 | S |
| ATOM | 4093 | C | CYS | I | 92 | 8.042 | 35.252 | −11.736 | 1.00 | 19.36 | C |
| ATOM | 4094 | O | CYS | I | 92 | 7.552 | 34.627 | −10.795 | 1.00 | 19.20 | O |
| ATOM | 4095 | N | ALA | I | 93 | 9.347 | 35.270 | −11.987 | 1.00 | 19.33 | N |
| ATOM | 4096 | CA | ALA | I | 93 | 10.285 | 34.480 | −11.195 | 1.00 | 19.44 | C |
| ATOM | 4097 | CB | ALA | I | 93 | 10.398 | 33.106 | −11.790 | 1.00 | 19.57 | C |
| ATOM | 4098 | C | ALA | I | 93 | 11.677 | 35.109 | −11.055 | 1.00 | 19.66 | C |
| ATOM | 4099 | O | ALA | I | 93 | 12.109 | 35.877 | −11.915 | 1.00 | 19.45 | O |
| ATOM | 4100 | N | ARG | I | 94 | 12.359 | 34.788 | −9.960 | 1.00 | 19.87 | N |
| ATOM | 4101 | CA | ARG | I | 94 | 13.774 | 35.129 | −9.785 | 1.00 | 20.75 | C |
| ATOM | 4102 | CB | ARG | I | 94 | 14.024 | 36.201 | −8.706 | 1.00 | 20.80 | C |
| ATOM | 4103 | CG | ARG | I | 94 | 15.513 | 36.528 | −8.587 | 1.00 | 23.23 | C |
| ATOM | 4104 | CD | ARG | I | 94 | 15.836 | 37.766 | −7.807 | 1.00 | 25.14 | C |
| ATOM | 4105 | NE | ARG | I | 94 | 16.099 | 37.494 | −6.404 | 1.00 | 28.89 | N |
| ATOM | 4106 | CZ | ARG | I | 94 | 17.192 | 37.859 | −5.726 | 1.00 | 31.33 | C |
| ATOM | 4107 | NH1 | ARG | I | 94 | 17.279 | 37.543 | −4.437 | 1.00 | 32.11 | N |
| ATOM | 4108 | NH2 | ARG | I | 94 | 18.190 | 38.529 | −6.303 | 1.00 | 31.00 | N |
| ATOM | 4109 | C | ARG | I | 94 | 14.525 | 33.865 | −9.441 | 1.00 | 20.57 | C |
| ATOM | 4110 | O | ARG | I | 94 | 14.285 | 33.272 | −8.397 | 1.00 | 20.44 | O |
| ATOM | 4111 | N | ARG | I | 95 | 15.440 | 33.482 | −10.327 | 1.00 | 21.04 | N |
| ATOM | 4112 | CA | ARG | I | 95 | 16.112 | 32.188 | −10.281 | 1.00 | 21.25 | C |
| ATOM | 4113 | CB | ARG | I | 95 | 15.641 | 31.314 | −11.436 | 1.00 | 21.11 | C |
| ATOM | 4114 | CG | ARG | I | 95 | 14.126 | 31.208 | −11.578 | 1.00 | 20.93 | C |
| ATOM | 4115 | CD | ARG | I | 95 | 13.753 | 30.102 | −12.556 | 1.00 | 21.34 | C |
| ATOM | 4116 | NE | ARG | I | 95 | 14.155 | 30.399 | −13.930 | 1.00 | 21.46 | N |
| ATOM | 4117 | CZ | ARG | I | 95 | 14.536 | 29.484 | −14.818 | 1.00 | 22.09 | C |
| ATOM | 4118 | NH1 | ARG | I | 95 | 14.602 | 28.199 | −14.484 | 1.00 | 21.84 | N |
| ATOM | 4119 | NH2 | ARG | I | 95 | 14.874 | 29.860 | −16.038 | 1.00 | 22.07 | N |
| ATOM | 4120 | C | ARG | I | 95 | 17.638 | 32.289 | −10.339 | 1.00 | 21.85 | C |
| ATOM | 4121 | O | ARG | I | 95 | 18.342 | 31.458 | −9.758 | 1.00 | 22.81 | O |
| ATOM | 4122 | N | PHE | I | 96 | 18.158 | 33.291 | −11.036 | 1.00 | 21.85 | N |
| ATOM | 4123 | CA | PHE | I | 96 | 19.607 | 33.423 | −11.205 | 1.00 | 21.84 | C |
| ATOM | 4124 | CB | PHE | I | 96 | 19.994 | 33.107 | −12.646 | 1.00 | 21.38 | C |
| ATOM | 4125 | CG | PHE | I | 96 | 19.310 | 31.906 | −13.218 | 1.00 | 20.20 | C |
| ATOM | 4126 | CD1 | PHE | I | 96 | 19.603 | 30.631 | −12.755 | 1.00 | 18.41 | C |
| ATOM | 4127 | CE1 | PHE | I | 96 | 18.977 | 29.518 | −13.309 | 1.00 | 18.12 | C |
| ATOM | 4128 | CZ | PHE | I | 96 | 18.060 | 29.675 | −14.342 | 1.00 | 17.33 | C |
| ATOM | 4129 | CE2 | PHE | I | 96 | 17.773 | 30.935 | −14.817 | 1.00 | 17.35 | C |
| ATOM | 4130 | CD2 | PHE | I | 96 | 18.397 | 32.046 | −14.258 | 1.00 | 18.86 | C |
| ATOM | 4131 | C | PHE | I | 96 | 20.162 | 34.815 | −10.875 | 1.00 | 22.23 | C |
| ATOM | 4132 | O | PHE | I | 96 | 19.414 | 35.783 | −10.734 | 1.00 | 22.17 | O |
| ATOM | 4133 | N | GLY | I | 97 | 21.491 | 34.886 | −10.760 | 1.00 | 22.70 | N |
| ATOM | 4134 | CA | GLY | I | 97 | 22.248 | 36.152 | −10.796 | 1.00 | 22.66 | C |
| ATOM | 4135 | C | GLY | I | 97 | 23.142 | 36.051 | −12.019 | 1.00 | 22.83 | C |
| ATOM | 4136 | O | GLY | I | 97 | 22.919 | 35.198 | −12.875 | 1.00 | 22.52 | O |
| ATOM | 4137 | N | TYR | I | 98 | 24.159 | 36.896 | −12.123 | 1.00 | 23.22 | N |
| ATOM | 4138 | CA | TYR | I | 98 | 25.036 | 36.800 | −13.283 | 1.00 | 23.47 | C |
| ATOM | 4139 | CB | TYR | I | 98 | 25.870 | 38.078 | −13.493 | 1.00 | 23.80 | C |
| ATOM | 4140 | CG | TYR | I | 98 | 26.584 | 38.079 | −14.836 | 1.00 | 25.79 | C |
| ATOM | 4141 | CD1 | TYR | I | 98 | 25.870 | 38.279 | −16.022 | 1.00 | 27.82 | C |
| ATOM | 4142 | CE1 | TYR | I | 98 | 26.510 | 38.265 | −17.269 | 1.00 | 28.83 | C |
| ATOM | 4143 | CZ | TYR | I | 98 | 27.869 | 38.036 | −17.332 | 1.00 | 30.45 | C |
| ATOM | 4144 | OH | TYR | I | 98 | 28.481 | 38.010 | −18.575 | 1.00 | 32.82 | O |
| ATOM | 4145 | CE2 | TYR | I | 98 | 28.608 | 37.812 | −16.162 | 1.00 | 28.46 | C |
| ATOM | 4146 | CD2 | TYR | I | 98 | 27.963 | 37.838 | −14.928 | 1.00 | 26.76 | C |
| ATOM | 4147 | C | TYR | I | 98 | 25.909 | 35.544 | −13.181 | 1.00 | 23.22 | C |
| ATOM | 4148 | O | TYR | I | 98 | 26.104 | 35.009 | −12.094 | 1.00 | 23.20 | O |
| ATOM | 4149 | N | TYR | I | 99 | 26.397 | 35.063 | −14.322 | 1.00 | 23.06 | N |
| ATOM | 4150 | CA | TYR | I | 99 | 27.265 | 33.900 | −14.376 | 1.00 | 23.27 | C |
| ATOM | 4151 | CB | TYR | I | 99 | 27.708 | 33.638 | −15.810 | 1.00 | 23.38 | C |
| ATOM | 4152 | CG | TYR | I | 99 | 26.591 | 33.243 | −16.753 | 1.00 | 24.22 | C |
| ATOM | 4153 | CD1 | TYR | I | 99 | 26.033 | 31.968 | −16.705 | 1.00 | 25.12 | C |
| ATOM | 4154 | CE1 | TYR | I | 99 | 25.008 | 31.588 | −17.573 | 1.00 | 25.34 | C |
| ATOM | 4155 | CZ | TYR | I | 99 | 24.544 | 32.479 | −18.518 | 1.00 | 25.49 | C |
| ATOM | 4156 | OH | TYR | I | 99 | 23.531 | 32.096 | −19.372 | 1.00 | 26.00 | O |
| ATOM | 4157 | CE2 | TYR | I | 99 | 25.079 | 33.758 | −18.592 | 1.00 | 25.45 | C |
| ATOM | 4158 | CD2 | TYR | I | 99 | 26.107 | 34.134 | −17.707 | 1.00 | 25.28 | C |
| ATOM | 4159 | C | TYR | I | 99 | 28.491 | 34.077 | −13.485 | 1.00 | 23.35 | C |
| ATOM | 4160 | O | TYR | I | 99 | 29.081 | 35.168 | −13.415 | 1.00 | 23.79 | O |
| ATOM | 4161 | N | GLY | I | 100 | 28.872 | 32.999 | −12.807 | 1.00 | 22.86 | N |
| ATOM | 4162 | CA | GLY | I | 100 | 29.970 | 33.049 | −11.858 | 1.00 | 22.44 | C |
| ATOM | 4163 | C | GLY | I | 100 | 29.607 | 33.641 | −10.503 | 1.00 | 22.16 | C |
| ATOM | 4164 | O | GLY | I | 100 | 30.451 | 33.724 | −9.620 | 1.00 | 22.23 | O |
| ATOM | 4165 | N | SER | I | 100A | 28.357 | 34.053 | −10.324 | 1.00 | 21.88 | N |
| ATOM | 4166 | CA | SER | I | 100A | 27.929 | 34.607 | −9.035 | 1.00 | 21.97 | C |
| ATOM | 4167 | CB | SER | I | 100A | 26.663 | 35.458 | −9.195 | 1.00 | 21.93 | C |
| ATOM | 4168 | OG | SER | I | 100A | 25.569 | 34.667 | −9.645 | 1.00 | 21.55 | O |
| ATOM | 4169 | C | SER | I | 100A | 27.680 | 33.528 | −7.972 | 1.00 | 22.06 | C |
| ATOM | 4170 | O | SER | I | 100A | 27.652 | 33.837 | −6.780 | 1.00 | 22.46 | O |
| ATOM | 4171 | N | GLY | I | 100B | 27.479 | 32.282 | −8.403 | 1.00 | 21.58 | N |
| ATOM | 4172 | CA | GLY | I | 100B | 27.091 | 31.206 | −7.499 | 1.00 | 21.60 | C |
| ATOM | 4173 | C | GLY | I | 100B | 25.627 | 31.278 | −7.049 | 1.00 | 21.88 | C |
| ATOM | 4174 | O | GLY | I | 100B | 25.177 | 30.432 | −6.263 | 1.00 | 21.69 | O |
| ATOM | 4175 | N | ASN | I | 100C | 24.886 | 32.268 | −7.561 | 1.00 | 21.25 | N |
| ATOM | 4176 | CA | ASN | I | 100C | 23.530 | 32.548 | −7.099 | 1.00 | 21.18 | C |
| ATOM | 4177 | CB | ASN | I | 100C | 23.178 | 34.034 | −7.269 | 1.00 | 21.53 | C |
| ATOM | 4178 | CG | ASN | I | 100C | 23.888 | 34.924 | −6.266 | 1.00 | 22.83 | C |
| ATOM | 4179 | OD1 | ASN | I | 100C | 24.234 | 34.490 | −5.167 | 1.00 | 24.81 | O |
| ATOM | 4180 | ND2 | ASN | I | 100C | 24.117 | 36.174 | −6.647 | 1.00 | 22.58 | N |
| ATOM | 4181 | C | ASN | I | 100C | 22.473 | 31.710 | −7.792 | 1.00 | 20.69 | C |
| ATOM | 4182 | O | ASN | I | 100C | 22.286 | 31.800 | −9.005 | 1.00 | 20.50 | O |
| ATOM | 4183 | N | TYR | I | 100D | 21.785 | 30.898 | −7.000 | 1.00 | 19.97 | N |
| ATOM | 4184 | CA | TYR | I | 100D | 20.641 | 30.144 | −7.463 | 1.00 | 19.51 | C |
| ATOM | 4185 | CB | TYR | I | 100D | 20.988 | 28.657 | −7.611 | 1.00 | 18.96 | C |
| ATOM | 4186 | CG | TYR | I | 100D | 21.941 | 28.458 | −8.758 | 1.00 | 19.66 | C |
| ATOM | 4187 | CD1 | TYR | I | 100D | 21.467 | 28.313 | −10.064 | 1.00 | 18.59 | C |
| ATOM | 4188 | CE1 | TYR | I | 100D | 22.344 | 28.181 | −11.126 | 1.00 | 19.06 | C |
| ATOM | 4189 | CZ | TYR | I | 100D | 23.712 | 28.188 | −10.894 | 1.00 | 19.40 | C |
| ATOM | 4190 | OH | TYR | I | 100D | 24.577 | 28.054 | −11.950 | 1.00 | 20.82 | O |
| ATOM | 4191 | CE2 | TYR | I | 100D | 24.211 | 28.336 | −9.620 | 1.00 | 18.99 | C |
| ATOM | 4192 | CD2 | TYR | I | 100D | 23.326 | 28.477 | −8.554 | 1.00 | 19.95 | C |
| ATOM | 4193 | C | TYR | I | 100D | 19.499 | 30.376 | −6.499 | 1.00 | 19.23 | C |
| ATOM | 4194 | O | TYR | I | 100D | 19.587 | 29.979 | −5.337 | 1.00 | 20.16 | O |
| ATOM | 4195 | N | PHE | I | 100E | 18.447 | 31.041 | −6.978 | 1.00 | 18.31 | N |
| ATOM | 4196 | CA | PHE | I | 100E | 17.251 | 31.287 | −6.183 | 1.00 | 17.74 | C |
| ATOM | 4197 | CB | PHE | I | 100E | 16.905 | 32.769 | −6.134 | 1.00 | 17.67 | C |
| ATOM | 4198 | CG | PHE | I | 100E | 18.077 | 33.676 | −6.000 | 1.00 | 18.00 | C |
| ATOM | 4199 | CD1 | PHE | I | 100E | 18.629 | 33.941 | −4.749 | 1.00 | 18.08 | C |
| ATOM | 4200 | CE1 | PHE | I | 100E | 19.719 | 34.805 | −4.613 | 1.00 | 17.55 | C |
| ATOM | 4201 | CZ | PHE | I | 100E | 20.234 | 35.432 | −5.739 | 1.00 | 18.15 | C |
| ATOM | 4202 | CE2 | PHE | I | 100E | 19.675 | 35.179 | −7.003 | 1.00 | 17.45 | C |
| ATOM | 4203 | CD2 | PHE | I | 100E | 18.607 | 34.309 | −7.123 | 1.00 | 17.30 | C |
| ATOM | 4204 | C | PHE | I | 100E | 16.059 | 30.559 | −6.787 | 1.00 | 17.38 | C |
| ATOM | 4205 | O | PHE | I | 100E | 16.154 | 30.010 | −7.884 | 1.00 | 17.91 | O |
| ATOM | 4206 | N | ASP | I | 101 | 14.938 | 30.589 | −6.075 | 1.00 | 16.55 | N |
| ATOM | 4207 | CA | ASP | I | 101 | 13.711 | 29.967 | −6.522 | 1.00 | 16.15 | C |
| ATOM | 4208 | CB | ASP | I | 101 | 13.643 | 28.512 | −6.060 | 1.00 | 15.88 | C |
| ATOM | 4209 | CG | ASP | I | 101 | 12.540 | 27.736 | −6.762 | 1.00 | 16.96 | C |
| ATOM | 4210 | OD1 | ASP | I | 101 | 12.441 | 27.838 | −8.011 | 1.00 | 16.86 | O |
| ATOM | 4211 | OD2 | ASP | I | 101 | 11.768 | 27.036 | −6.067 | 1.00 | 17.73 | O |
| ATOM | 4212 | C | ASP | I | 101 | 12.475 | 30.734 | −6.034 | 1.00 | 15.74 | C |
| ATOM | 4213 | O | ASP | I | 101 | 11.608 | 30.169 | −5.370 | 1.00 | 15.05 | O |
| ATOM | 4214 | N | TYR | I | 102 | 12.401 | 32.015 | −6.392 | 1.00 | 15.81 | N |
| ATOM | 4215 | CA | TYR | I | 102 | 11.287 | 32.894 | −6.002 | 1.00 | 16.21 | C |
| ATOM | 4216 | CB | TYR | I | 102 | 11.802 | 34.279 | −5.603 | 1.00 | 16.15 | C |
| ATOM | 4217 | CG | TYR | I | 102 | 12.746 | 34.227 | −4.426 | 1.00 | 18.09 | C |
| ATOM | 4218 | CD1 | TYR | I | 102 | 12.294 | 33.793 | −3.160 | 1.00 | 19.55 | C |
| ATOM | 4219 | CE1 | TYR | I | 102 | 13.164 | 33.725 | −2.061 | 1.00 | 19.23 | C |
| ATOM | 4220 | CZ | TYR | I | 102 | 14.501 | 34.094 | −2.223 | 1.00 | 20.66 | C |
| ATOM | 4221 | OH | TYR | I | 102 | 15.347 | 34.035 | −1.139 | 1.00 | 22.01 | O |
| ATOM | 4222 | CE2 | TYR | I | 102 | 14.981 | 34.513 | −3.470 | 1.00 | 19.25 | C |
| ATOM | 4223 | CD2 | TYR | I | 102 | 14.097 | 34.575 | −4.568 | 1.00 | 17.56 | C |
| ATOM | 4224 | C | TYR | I | 102 | 10.283 | 33.027 | −7.122 | 1.00 | 15.92 | C |
| ATOM | 4225 | O | TYR | I | 102 | 10.650 | 33.374 | −8.230 | 1.00 | 16.60 | O |
| ATOM | 4226 | N | TRP | I | 103 | 9.023 | 32.737 | −6.831 | 1.00 | 15.50 | N |
| ATOM | 4227 | CA | TRP | I | 103 | 7.980 | 32.761 | −7.843 | 1.00 | 15.33 | C |
| ATOM | 4228 | CB | TRP | I | 103 | 7.491 | 31.342 | −8.151 | 1.00 | 14.80 | C |
| ATOM | 4229 | CG | TRP | I | 103 | 8.491 | 30.461 | −8.825 | 1.00 | 14.32 | C |
| ATOM | 4230 | CD1 | TRP | I | 103 | 9.551 | 29.804 | −8.238 | 1.00 | 14.63 | C |
| ATOM | 4231 | NE1 | TRP | I | 103 | 10.240 | 29.069 | −9.189 | 1.00 | 13.54 | N |
| ATOM | 4232 | CE2 | TRP | I | 103 | 9.631 | 29.247 | −10.405 | 1.00 | 13.39 | C |
| ATOM | 4233 | CD2 | TRP | I | 103 | 8.524 | 30.109 | −10.213 | 1.00 | 13.61 | C |
| ATOM | 4234 | CE3 | TRP | I | 103 | 7.725 | 30.433 | −11.316 | 1.00 | 12.68 | C |
| ATOM | 4235 | CZ3 | TRP | I | 103 | 8.045 | 29.903 | −12.545 | 1.00 | 10.40 | C |
| ATOM | 4236 | CH2 | TRP | I | 103 | 9.152 | 29.059 | −12.707 | 1.00 | 11.18 | C |
| ATOM | 4237 | CZ2 | TRP | I | 103 | 9.954 | 28.720 | −11.655 | 1.00 | 12.62 | C |
| ATOM | 4238 | C | TRP | I | 103 | 6.799 | 33.562 | −7.345 | 1.00 | 15.57 | C |
| ATOM | 4239 | O | TRP | I | 103 | 6.431 | 33.474 | −6.172 | 1.00 | 15.12 | O |
| ATOM | 4240 | N | GLY | I | 104 | 6.189 | 34.323 | −8.246 | 1.00 | 15.68 | N |
| ATOM | 4241 | CA | GLY | I | 104 | 4.921 | 34.961 | −7.945 | 1.00 | 16.31 | C |
| ATOM | 4242 | C | GLY | I | 104 | 3.843 | 33.896 | −7.893 | 1.00 | 16.99 | C |
| ATOM | 4243 | O | GLY | I | 104 | 4.061 | 32.760 | −8.335 | 1.00 | 16.99 | O |
| ATOM | 4244 | N | GLN | I | 105 | 2.675 | 34.248 | −7.365 | 1.00 | 17.45 | N |
| ATOM | 4245 | CA | GLN | I | 105 | 1.576 | 33.285 | −7.281 | 1.00 | 18.12 | C |
| ATOM | 4246 | CB | GLN | I | 105 | 0.523 | 33.749 | −6.259 | 1.00 | 17.98 | C |
| ATOM | 4247 | CG | GLN | I | 105 | −0.404 | 34.848 | −6.781 | 1.00 | 17.50 | C |
| ATOM | 4248 | CD | GLN | I | 105 | 0.198 | 36.245 | −6.681 | 1.00 | 16.65 | C |
| ATOM | 4249 | OE1 | GLN | I | 105 | 1.419 | 36.423 | −6.597 | 1.00 | 15.77 | O |
| ATOM | 4250 | NE2 | GLN | I | 105 | −0.667 | 37.247 | −6.703 | 1.00 | 15.15 | N |
| ATOM | 4251 | C | GLN | I | 105 | 0.925 | 33.052 | −8.657 | 1.00 | 18.56 | C |
| ATOM | 4252 | O | GLN | I | 105 | 0.053 | 32.188 | −8.795 | 1.00 | 18.42 | O |
| ATOM | 4253 | N | GLY | I | 106 | 1.346 | 33.837 | −9.657 | 1.00 | 18.84 | N |
| ATOM | 4254 | CA | GLY | I | 106 | 0.783 | 33.772 | −11.003 | 1.00 | 18.74 | C |
| ATOM | 4255 | C | GLY | I | 106 | −0.432 | 34.662 | −11.208 | 1.00 | 18.82 | C |
| ATOM | 4256 | O | GLY | I | 106 | −1.228 | 34.885 | −10.292 | 1.00 | 18.23 | O |
| ATOM | 4257 | N | THR | I | 107 | −0.556 | 35.189 | −12.420 | 1.00 | 19.42 | N |
| ATOM | 4258 | CA | THR | I | 107 | −1.721 | 35.966 | −12.815 | 1.00 | 19.84 | C |
| ATOM | 4259 | CB | THR | I | 107 | −1.334 | 37.419 | −13.177 | 1.00 | 19.62 | C |
| ATOM | 4260 | OG1 | THR | I | 107 | −0.968 | 38.114 | −11.985 | 1.00 | 18.73 | O |
| ATOM | 4261 | CG2 | THR | I | 107 | −2.501 | 38.174 | −13.825 | 1.00 | 19.50 | C |
| ATOM | 4262 | C | THR | I | 107 | −2.376 | 35.263 | −13.986 | 1.00 | 20.57 | C |
| ATOM | 4263 | O | THR | I | 107 | −1.707 | 34.933 | −14.972 | 1.00 | 20.57 | O |
| ATOM | 4264 | N | MET | I | 108 | −3.674 | 35.000 | −13.848 | 1.00 | 21.92 | N |
| ATOM | 4265 | CA | MET | I | 108 | −4.471 | 34.342 | −14.893 | 1.00 | 23.23 | C |
| ATOM | 4266 | CB | MET | I | 108 | −5.676 | 33.610 | −14.281 | 1.00 | 23.67 | C |
| ATOM | 4267 | CG | MET | I | 108 | −6.435 | 32.659 | −15.244 | 1.00 | 26.73 | C |
| ATOM | 4268 | SD | MET | I | 108 | −5.400 | 31.357 | −15.992 | 1.00 | 33.24 | S |
| ATOM | 4269 | CE | MET | I | 108 | −6.032 | 31.366 | −17.676 | 1.00 | 31.39 | C |
| ATOM | 4270 | C | MET | I | 108 | −4.955 | 35.396 | −15.878 | 1.00 | 23.34 | C |
| ATOM | 4271 | O | MET | I | 108 | −5.546 | 36.403 | −15.476 | 1.00 | 23.73 | O |
| ATOM | 4272 | N | VAL | I | 109 | −4.699 | 35.176 | −17.161 | 1.00 | 23.21 | N |
| ATOM | 4273 | CA | VAL | I | 109 | −5.191 | 36.092 | −18.187 | 1.00 | 23.32 | C |
| ATOM | 4274 | CB | VAL | I | 109 | −4.039 | 36.785 | −18.966 | 1.00 | 23.21 | C |
| ATOM | 4275 | CG1 | VAL | I | 109 | −4.579 | 37.650 | −20.081 | 1.00 | 22.53 | C |
| ATOM | 4276 | CG2 | VAL | I | 109 | −3.198 | 37.643 | −18.028 | 1.00 | 22.93 | C |
| ATOM | 4277 | C | VAL | I | 109 | −6.106 | 35.326 | −19.132 | 1.00 | 23.47 | C |
| ATOM | 4278 | O | VAL | I | 109 | −5.708 | 34.313 | −19.704 | 1.00 | 23.90 | O |
| ATOM | 4279 | N | THR | I | 110 | −7.340 | 35.804 | −19.250 | 1.00 | 23.29 | N |
| ATOM | 4280 | CA | THR | I | 110 | −8.310 | 35.268 | −20.174 | 1.00 | 23.16 | C |
| ATOM | 4281 | CB | THR | I | 110 | −9.606 | 34.894 | −19.456 | 1.00 | 23.20 | C |
| ATOM | 4282 | OG1 | THR | I | 110 | −9.312 | 33.970 | −18.406 | 1.00 | 23.50 | O |
| ATOM | 4283 | CG2 | THR | I | 110 | −10.581 | 34.250 | −20.416 | 1.00 | 23.35 | C |
| ATOM | 4284 | C | THR | I | 110 | −8.605 | 36.330 | −21.215 | 1.00 | 23.27 | C |
| ATOM | 4285 | O | THR | I | 110 | −8.915 | 37.474 | −20.880 | 1.00 | 23.14 | O |
| ATOM | 4286 | N | VAL | I | 111 | −8.486 | 35.943 | −22.479 | 1.00 | 23.50 | N |
| ATOM | 4287 | CA | VAL | I | 111 | −8.810 | 36.811 | −23.600 | 1.00 | 23.67 | C |
| ATOM | 4288 | CB | VAL | I | 111 | −7.620 | 36.991 | −24.556 | 1.00 | 23.85 | C |
| ATOM | 4289 | CG1 | VAL | I | 111 | −7.996 | 37.955 | −25.690 | 1.00 | 24.21 | C |
| ATOM | 4290 | CG2 | VAL | I | 111 | −6.369 | 37.483 | −23.804 | 1.00 | 22.52 | C |
| ATOM | 4291 | C | VAL | I | 111 | −9.959 | 36.175 | −24.361 | 1.00 | 24.14 | C |
| ATOM | 4292 | O | VAL | I | 111 | −9.859 | 35.023 | −24.803 | 1.00 | 24.53 | O |
| ATOM | 4293 | N | SER | I | 112 | −11.056 | 36.915 | −24.501 | 1.00 | 24.26 | N |
| ATOM | 4294 | CA | SER | I | 112 | −12.259 | 36.383 | −25.124 | 1.00 | 24.30 | C |
| ATOM | 4295 | CB | SER | I | 112 | −12.923 | 35.383 | −24.197 | 1.00 | 23.94 | C |
| ATOM | 4296 | OG | SER | I | 112 | −14.138 | 34.909 | −24.734 | 1.00 | 24.04 | O |
| ATOM | 4297 | C | SER | I | 112 | −13.237 | 37.498 | −25.450 | 1.00 | 24.91 | C |
| ATOM | 4298 | O | SER | I | 112 | −13.355 | 38.474 | −24.703 | 1.00 | 25.30 | O |
| ATOM | 4299 | N | SER | I | 113 | −13.955 | 37.344 | −26.556 | 1.00 | 25.09 | N |
| ATOM | 4300 | CA | SER | I | 113 | −14.933 | 38.349 | −26.943 | 1.00 | 25.61 | C |
| ATOM | 4301 | CB | SER | I | 113 | −15.156 | 38.346 | −28.446 | 1.00 | 25.52 | C |
| ATOM | 4302 | OG | SER | I | 113 | −15.734 | 37.113 | −28.820 | 1.00 | 28.16 | O |
| ATOM | 4303 | C | SER | I | 113 | −16.240 | 38.112 | −26.203 | 1.00 | 25.18 | C |
| ATOM | 4304 | O | SER | I | 113 | −17.174 | 38.889 | −26.337 | 1.00 | 25.01 | O |
| ATOM | 4305 | N | ALA | I | 114 | −16.282 | 37.047 | −25.408 | 1.00 | 24.87 | N |
| ATOM | 4306 | CA | ALA | I | 114 | −17.424 | 36.761 | −24.553 | 1.00 | 25.04 | C |
| ATOM | 4307 | CB | ALA | I | 114 | −17.277 | 35.392 | −23.922 | 1.00 | 24.75 | C |
| ATOM | 4308 | C | ALA | I | 114 | −17.579 | 37.830 | −23.475 | 1.00 | 25.31 | C |
| ATOM | 4309 | O | ALA | I | 114 | −16.642 | 38.579 | −23.195 | 1.00 | 25.60 | O |
| ATOM | 4310 | N | SER | I | 115 | −18.762 | 37.893 | −22.876 | 1.00 | 25.52 | N |
| ATOM | 4311 | CA | SER | I | 115 | −19.087 | 38.920 | −21.904 | 1.00 | 26.51 | C |
| ATOM | 4312 | CB | SER | I | 115 | −20.148 | 39.869 | −22.468 | 1.00 | 26.83 | C |
| ATOM | 4313 | OG | SER | I | 115 | −19.665 | 40.585 | −23.601 | 1.00 | 28.68 | O |
| ATOM | 4314 | C | SER | I | 115 | −19.606 | 38.298 | −20.622 | 1.00 | 26.82 | C |
| ATOM | 4315 | O | SER | I | 115 | −20.314 | 37.286 | −20.655 | 1.00 | 27.31 | O |
| ATOM | 4316 | N | THR | I | 116 | −19.274 | 38.921 | −19.495 | 1.00 | 26.87 | N |
| ATOM | 4317 | CA | THR | I | 116 | −19.675 | 38.427 | −18.185 | 1.00 | 27.09 | C |
| ATOM | 4318 | CB | THR | I | 116 | −19.421 | 39.504 | −17.107 | 1.00 | 26.96 | C |
| ATOM | 4319 | OG1 | THR | I | 116 | −18.015 | 39.745 | −17.041 | 1.00 | 26.96 | O |
| ATOM | 4320 | CG2 | THR | I | 116 | −19.886 | 39.048 | −15.739 | 1.00 | 26.43 | C |
| ATOM | 4321 | C | THR | I | 116 | −21.122 | 37.951 | −18.184 | 1.00 | 27.51 | C |
| ATOM | 4322 | O | THR | I | 116 | −22.014 | 38.672 | −18.601 | 1.00 | 27.78 | O |
| ATOM | 4323 | N | LYS | I | 117 | −21.332 | 36.710 | −17.753 | 1.00 | 28.42 | N |
| ATOM | 4324 | CA | LYS | I | 117 | −22.671 | 36.112 | −17.637 | 1.00 | 28.75 | C |
| ATOM | 4325 | CB | LYS | I | 117 | −23.143 | 35.494 | −18.965 | 1.00 | 29.03 | C |
| ATOM | 4326 | CG | LYS | I | 117 | −24.528 | 34.835 | −18.861 | 1.00 | 30.44 | C |
| ATOM | 4327 | CD | LYS | I | 117 | −24.929 | 34.060 | −20.107 | 1.00 | 33.42 | C |
| ATOM | 4328 | CE | LYS | I | 117 | −26.263 | 33.325 | −19.900 | 1.00 | 35.18 | C |
| ATOM | 4329 | NZ | LYS | I | 117 | −26.231 | 32.281 | −18.786 | 1.00 | 36.66 | N |
| ATOM | 4330 | C | LYS | I | 117 | −22.664 | 35.036 | −16.566 | 1.00 | 28.67 | C |
| ATOM | 4331 | O | LYS | I | 117 | −21.795 | 34.162 | −16.559 | 1.00 | 28.64 | O |
| ATOM | 4332 | N | GLY | I | 118 | −23.647 | 35.103 | −15.673 | 1.00 | 28.75 | N |
| ATOM | 4333 | CA | GLY | I | 118 | −23.811 | 34.125 | −14.602 | 1.00 | 28.92 | C |
| ATOM | 4334 | C | GLY | I | 118 | −24.345 | 32.784 | −15.068 | 1.00 | 28.83 | C |
| ATOM | 4335 | O | GLY | I | 118 | −25.031 | 32.709 | −16.080 | 1.00 | 28.72 | O |
| ATOM | 4336 | N | PRO | I | 119 | −24.032 | 31.715 | −14.321 | 1.00 | 29.09 | N |
| ATOM | 4337 | CA | PRO | I | 119 | −24.400 | 30.354 | −14.702 | 1.00 | 29.46 | C |
| ATOM | 4338 | CB | PRO | I | 119 | −23.557 | 29.496 | −13.760 | 1.00 | 29.34 | C |
| ATOM | 4339 | CG | PRO | I | 119 | −23.392 | 30.328 | −12.546 | 1.00 | 29.08 | C |
| ATOM | 4340 | CD | PRO | I | 119 | −23.275 | 31.743 | −13.055 | 1.00 | 29.18 | C |
| ATOM | 4341 | C | PRO | I | 119 | −25.866 | 30.027 | −14.468 | 1.00 | 29.83 | C |
| ATOM | 4342 | O | PRO | I | 119 | −26.542 | 30.686 | −13.679 | 1.00 | 29.97 | O |
| ATOM | 4343 | N | SER | I | 120 | −26.342 | 29.002 | −15.161 | 1.00 | 30.24 | N |
| ATOM | 4344 | CA | SER | I | 120 | −27.602 | 28.372 | −14.824 | 1.00 | 30.61 | C |
| ATOM | 4345 | CB | SER | I | 120 | −28.420 | 28.108 | −16.084 | 1.00 | 30.76 | C |
| ATOM | 4346 | OG | SER | I | 120 | −28.582 | 29.303 | −16.826 | 1.00 | 32.17 | O |
| ATOM | 4347 | C | SER | I | 120 | −27.252 | 27.069 | −14.128 | 1.00 | 30.44 | C |
| ATOM | 4348 | O | SER | I | 120 | −26.343 | 26.365 | −14.563 | 1.00 | 30.47 | O |
| ATOM | 4349 | N | VAL | I | 121 | −27.955 | 26.769 | −13.039 | 1.00 | 30.45 | N |
| ATOM | 4350 | CA | VAL | I | 121 | −27.704 | 25.555 | −12.272 | 1.00 | 30.83 | C |
| ATOM | 4351 | CB | VAL | I | 121 | −27.382 | 25.864 | −10.783 | 1.00 | 30.79 | C |
| ATOM | 4352 | CG1 | VAL | I | 121 | −27.083 | 24.587 | −10.033 | 1.00 | 30.35 | C |
| ATOM | 4353 | CG2 | VAL | I | 121 | −26.198 | 26.826 | −10.665 | 1.00 | 30.49 | C |
| ATOM | 4354 | C | VAL | I | 121 | −28.854 | 24.537 | −12.387 | 1.00 | 31.23 | C |
| ATOM | 4355 | O | VAL | I | 121 | −29.958 | 24.749 | −11.886 | 1.00 | 30.89 | O |
| ATOM | 4356 | N | PHE | I | 122 | −28.566 | 23.422 | −13.048 | 1.00 | 31.78 | N |
| ATOM | 4357 | CA | PHE | I | 122 | −29.541 | 22.353 | −13.210 | 1.00 | 32.17 | C |
| ATOM | 4358 | CB | PHE | I | 122 | −29.721 | 22.026 | −14.690 | 1.00 | 31.99 | C |
| ATOM | 4359 | CG | PHE | I | 122 | −30.068 | 23.221 | −15.525 | 1.00 | 32.14 | C |
| ATOM | 4360 | CD1 | PHE | I | 122 | −31.235 | 23.937 | −15.283 | 1.00 | 31.83 | C |
| ATOM | 4361 | CE1 | PHE | I | 122 | −31.561 | 25.039 | −16.046 | 1.00 | 31.99 | C |
| ATOM | 4362 | CZ | PHE | I | 122 | −30.714 | 25.443 | −17.066 | 1.00 | 32.83 | C |
| ATOM | 4363 | CE2 | PHE | I | 122 | −29.545 | 24.738 | −17.318 | 1.00 | 32.43 | C |
| ATOM | 4364 | CD2 | PHE | I | 122 | −29.230 | 23.634 | −16.549 | 1.00 | 32.11 | C |
| ATOM | 4365 | C | PHE | I | 122 | −29.145 | 21.109 | −12.425 | 1.00 | 32.63 | C |
| ATOM | 4366 | O | PHE | I | 122 | −27.974 | 20.728 | −12.409 | 1.00 | 32.13 | O |
| ATOM | 4367 | N | PRO | I | 123 | −30.125 | 20.477 | −11.753 | 1.00 | 33.46 | N |
| ATOM | 4368 | CA | PRO | I | 123 | −29.822 | 19.267 | −10.989 | 1.00 | 33.80 | C |
| ATOM | 4369 | CB | PRO | I | 123 | −31.096 | 19.033 | −10.176 | 1.00 | 33.79 | C |
| ATOM | 4370 | CG | PRO | I | 123 | −32.193 | 19.678 | −10.970 | 1.00 | 33.33 | C |
| ATOM | 4371 | CD | PRO | I | 123 | −31.560 | 20.832 | −11.704 | 1.00 | 33.45 | C |
| ATOM | 4372 | C | PRO | I | 123 | −29.550 | 18.072 | −11.900 | 1.00 | 34.57 | C |
| ATOM | 4373 | O | PRO | I | 123 | −30.161 | 17.937 | −12.970 | 1.00 | 34.28 | O |
| ATOM | 4374 | N | LEU | I | 124 | −28.600 | 17.244 | −11.482 | 1.00 | 35.49 | N |
| ATOM | 4375 | CA | LEU | I | 124 | −28.387 | 15.938 | −12.074 | 1.00 | 36.71 | C |
| ATOM | 4376 | CB | LEU | I | 124 | −26.900 | 15.702 | −12.348 | 1.00 | 36.25 | C |
| ATOM | 4377 | CG | LEU | I | 124 | −26.220 | 16.719 | −13.267 | 1.00 | 36.08 | C |
| ATOM | 4378 | CD1 | LEU | I | 124 | −24.727 | 16.755 | −13.012 | 1.00 | 35.50 | C |
| ATOM | 4379 | CD2 | LEU | I | 124 | −26.509 | 16.443 | −14.737 | 1.00 | 34.70 | C |
| ATOM | 4380 | C | LEU | I | 124 | −28.930 | 14.957 | −11.049 | 1.00 | 37.69 | C |
| ATOM | 4381 | O | LEU | I | 124 | −28.274 | 14.673 | −10.047 | 1.00 | 37.79 | O |
| ATOM | 4382 | N | ALA | I | 125 | −30.149 | 14.479 | −11.281 | 1.00 | 39.25 | N |
| ATOM | 4383 | CA | ALA | I | 125 | −30.903 | 13.762 | −10.249 | 1.00 | 40.89 | C |
| ATOM | 4384 | CB | ALA | I | 125 | −32.391 | 13.993 | −10.430 | 1.00 | 40.53 | C |
| ATOM | 4385 | C | ALA | I | 125 | −30.589 | 12.264 | −10.197 | 1.00 | 42.31 | C |
| ATOM | 4386 | O | ALA | I | 125 | −30.253 | 11.664 | −11.224 | 1.00 | 42.47 | O |
| ATOM | 4387 | N | PRO | I | 126 | −30.685 | 11.659 | −8.994 | 1.00 | 43.66 | N |
| ATOM | 4388 | CA | PRO | I | 126 | −30.586 | 10.202 | −8.873 | 1.00 | 44.75 | C |
| ATOM | 4389 | CB | PRO | I | 126 | −30.449 | 9.984 | −7.360 | 1.00 | 44.71 | C |
| ATOM | 4390 | CG | PRO | I | 126 | −31.082 | 11.189 | −6.733 | 1.00 | 43.91 | C |
| ATOM | 4391 | CD | PRO | I | 126 | −30.782 | 12.319 | −7.673 | 1.00 | 43.69 | C |
| ATOM | 4392 | C | PRO | I | 126 | −31.843 | 9.503 | −9.377 | 1.00 | 45.88 | C |
| ATOM | 4393 | O | PRO | I | 126 | −32.952 | 10.000 | −9.156 | 1.00 | 45.98 | O |
| ATOM | 4394 | N | SER | I | 127 | −31.666 | 8.367 | −10.049 | 1.00 | 47.38 | N |
| ATOM | 4395 | CA | SER | I | 127 | −32.783 | 7.489 | −10.412 | 1.00 | 48.92 | C |
| ATOM | 4396 | CB | SER | I | 127 | −32.625 | 6.977 | −11.850 | 1.00 | 49.33 | C |
| ATOM | 4397 | OG | SER | I | 127 | −31.468 | 6.162 | −11.987 | 1.00 | 50.14 | O |
| ATOM | 4398 | C | SER | I | 127 | −32.842 | 6.327 | −9.420 | 1.00 | 49.69 | C |
| ATOM | 4399 | O | SER | I | 127 | −32.349 | 6.457 | −8.291 | 1.00 | 50.20 | O |
| ATOM | 4400 | N | SER | I | 128 | −33.451 | 5.208 | −9.827 | 1.00 | 50.35 | N |
| ATOM | 4401 | CA | SER | I | 128 | −33.458 | 3.969 | −9.022 | 1.00 | 50.86 | C |
| ATOM | 4402 | CB | SER | I | 128 | −34.199 | 4.158 | −7.679 | 1.00 | 51.16 | C |
| ATOM | 4403 | OG | SER | I | 128 | −35.477 | 4.757 | −7.850 | 1.00 | 50.99 | O |
| ATOM | 4404 | C | SER | I | 128 | −34.026 | 2.773 | −9.791 | 1.00 | 51.09 | C |
| ATOM | 4405 | O | SER | I | 128 | −33.594 | 2.471 | −10.907 | 1.00 | 51.13 | O |
| ATOM | 4406 | N | GLY | I | 133 | −26.829 | 0.815 | −7.331 | 1.00 | 50.31 | N |
| ATOM | 4407 | CA | GLY | I | 133 | −26.459 | −0.327 | −6.492 | 1.00 | 50.42 | C |
| ATOM | 4408 | C | GLY | I | 133 | −26.801 | −0.094 | −5.028 | 1.00 | 50.19 | C |
| ATOM | 4409 | O | GLY | I | 133 | −27.985 | −0.086 | −4.653 | 1.00 | 50.47 | O |
| ATOM | 4410 | N | GLY | I | 134 | −25.765 | 0.078 | −4.203 | 1.00 | 49.66 | N |
| ATOM | 4411 | CA | GLY | I | 134 | −25.920 | 0.513 | −2.801 | 1.00 | 48.92 | C |
| ATOM | 4412 | C | GLY | I | 134 | −25.585 | 1.994 | −2.597 | 1.00 | 48.29 | C |
| ATOM | 4413 | O | GLY | I | 134 | −25.771 | 2.550 | −1.506 | 1.00 | 48.27 | O |
| ATOM | 4414 | N | THR | I | 135 | −25.078 | 2.630 | −3.653 | 1.00 | 47.32 | N |
| ATOM | 4415 | CA | THR | I | 135 | −24.767 | 4.056 | −3.638 | 1.00 | 46.09 | C |
| ATOM | 4416 | CB | THR | I | 135 | −23.241 | 4.314 | −3.678 | 1.00 | 46.31 | C |
| ATOM | 4417 | OG1 | THR | I | 135 | −22.708 | 3.836 | −4.919 | 1.00 | 46.45 | O |
| ATOM | 4418 | CG2 | THR | I | 135 | −22.528 | 3.625 | −2.510 | 1.00 | 46.13 | C |
| ATOM | 4419 | C | THR | I | 135 | −25.414 | 4.753 | −4.836 | 1.00 | 45.11 | C |
| ATOM | 4420 | O | THR | I | 135 | −25.509 | 4.176 | −5.924 | 1.00 | 45.00 | O |
| ATOM | 4421 | N | ALA | I | 136 | −25.858 | 5.989 | −4.629 | 1.00 | 43.70 | N |
| ATOM | 4422 | CA | ALA | I | 136 | −26.424 | 6.802 | −5.704 | 1.00 | 42.36 | C |
| ATOM | 4423 | CB | ALA | I | 136 | −27.770 | 7.389 | −5.276 | 1.00 | 42.16 | C |
| ATOM | 4424 | C | ALA | I | 136 | −25.458 | 7.921 | −6.102 | 1.00 | 41.39 | C |
| ATOM | 4425 | O | ALA | I | 136 | −24.808 | 8.520 | −5.244 | 1.00 | 41.41 | O |
| ATOM | 4426 | N | ALA | I | 137 | −25.356 | 8.198 | −7.398 | 1.00 | 39.94 | N |
| ATOM | 4427 | CA | ALA | I | 137 | −24.664 | 9.404 | −7.838 | 1.00 | 38.74 | C |
| ATOM | 4428 | CB | ALA | I | 137 | −23.822 | 9.132 | −9.053 | 1.00 | 38.40 | C |
| ATOM | 4429 | C | ALA | I | 137 | −25.694 | 10.486 | −8.126 | 1.00 | 37.90 | C |
| ATOM | 4430 | O | ALA | I | 137 | −26.780 | 10.185 | −8.593 | 1.00 | 38.09 | O |
| ATOM | 4431 | N | LEU | I | 138 | −25.348 | 11.733 | −7.812 | 1.00 | 37.09 | N |
| ATOM | 4432 | CA | LEU | I | 138 | −26.168 | 12.915 | −8.103 | 1.00 | 36.09 | C |
| ATOM | 4433 | CB | LEU | I | 138 | −27.185 | 13.160 | −6.990 | 1.00 | 36.07 | C |
| ATOM | 4434 | CG | LEU | I | 138 | −26.660 | 13.396 | −5.576 | 1.00 | 35.93 | C |
| ATOM | 4435 | CD1 | LEU | I | 138 | −26.566 | 14.873 | −5.288 | 1.00 | 36.54 | C |
| ATOM | 4436 | CD2 | LEU | I | 138 | −27.598 | 12.741 | −4.589 | 1.00 | 36.46 | C |
| ATOM | 4437 | C | LEU | I | 138 | −25.258 | 14.131 | −8.261 | 1.00 | 35.67 | C |
| ATOM | 4438 | O | LEU | I | 138 | −24.084 | 14.076 | −7.882 | 1.00 | 35.81 | O |
| ATOM | 4439 | N | GLY | I | 139 | −25.782 | 15.225 | −8.809 | 1.00 | 34.66 | N |
| ATOM | 4440 | CA | GLY | I | 139 | −24.933 | 16.371 | −9.095 | 1.00 | 33.73 | C |
| ATOM | 4441 | C | GLY | I | 139 | −25.582 | 17.662 | −9.540 | 1.00 | 33.04 | C |
| ATOM | 4442 | O | GLY | I | 139 | −26.793 | 17.825 | −9.456 | 1.00 | 33.19 | O |
| ATOM | 4443 | N | CYS | I | 140 | −24.742 | 18.587 | −9.993 | 1.00 | 32.26 | N |
| ATOM | 4444 | CA | CYS | I | 140 | −25.179 | 19.874 | −10.532 | 1.00 | 31.54 | C |
| ATOM | 4445 | CB | CYS | I | 140 | −25.010 | 20.990 | −9.507 | 1.00 | 31.48 | C |
| ATOM | 4446 | SG | CYS | I | 140 | −26.319 | 20.982 | −8.337 | 1.00 | 32.14 | S |
| ATOM | 4447 | C | CYS | I | 140 | −24.401 | 20.220 | −11.786 | 1.00 | 30.84 | C |
| ATOM | 4448 | O | CYS | I | 140 | −23.165 | 20.102 | −11.827 | 1.00 | 31.29 | O |
| ATOM | 4449 | N | LEU | I | 141 | −25.143 | 20.624 | −12.807 | 1.00 | 29.60 | N |
| ATOM | 4450 | CA | LEU | I | 141 | −24.582 | 21.085 | −14.051 | 1.00 | 28.76 | C |
| ATOM | 4451 | CB | LEU | I | 141 | −25.445 | 20.607 | −15.218 | 1.00 | 28.56 | C |
| ATOM | 4452 | CG | LEU | I | 141 | −25.081 | 20.980 | −16.649 | 1.00 | 28.36 | C |
| ATOM | 4453 | CD1 | LEU | I | 141 | −23.697 | 20.507 | −17.028 | 1.00 | 27.71 | C |
| ATOM | 4454 | CD2 | LEU | I | 141 | −26.114 | 20.407 | −17.604 | 1.00 | 28.08 | C |
| ATOM | 4455 | C | LEU | I | 141 | −24.596 | 22.587 | −13.943 | 1.00 | 28.45 | C |
| ATOM | 4456 | O | LEU | I | 141 | −25.651 | 23.205 | −13.795 | 1.00 | 28.64 | O |
| ATOM | 4457 | N | VAL | I | 142 | −23.417 | 23.179 | −13.958 | 1.00 | 27.97 | N |
| ATOM | 4458 | CA | VAL | I | 142 | −23.311 | 24.615 | −13.824 | 1.00 | 27.43 | C |
| ATOM | 4459 | CB | VAL | I | 142 | −22.255 | 24.969 | −12.738 | 1.00 | 27.73 | C |
| ATOM | 4460 | CG1 | VAL | I | 142 | −22.152 | 26.480 | −12.508 | 1.00 | 26.88 | C |
| ATOM | 4461 | CG2 | VAL | I | 142 | −22.620 | 24.267 | −11.436 | 1.00 | 26.93 | C |
| ATOM | 4462 | C | VAL | I | 142 | −23.001 | 25.140 | −15.221 | 1.00 | 27.27 | C |
| ATOM | 4463 | O | VAL | I | 142 | −21.856 | 25.137 | −15.664 | 1.00 | 27.01 | O |
| ATOM | 4464 | N | LYS | I | 143 | −24.049 | 25.553 | −15.922 | 1.00 | 27.20 | N |
| ATOM | 4465 | CA | LYS | I | 143 | −23.971 | 25.784 | −17.363 | 1.00 | 27.38 | C |
| ATOM | 4466 | CB | LYS | I | 143 | −25.094 | 25.003 | −18.068 | 1.00 | 27.92 | C |
| ATOM | 4467 | CG | LYS | I | 143 | −25.208 | 25.233 | −19.581 | 1.00 | 28.02 | C |
| ATOM | 4468 | CD | LYS | I | 143 | −25.556 | 23.960 | −20.331 | 1.00 | 28.46 | C |
| ATOM | 4469 | CE | LYS | I | 143 | −26.012 | 24.253 | −21.763 | 1.00 | 28.22 | C |
| ATOM | 4470 | NZ | LYS | I | 143 | −24.888 | 24.524 | −22.690 | 1.00 | 27.36 | N |
| ATOM | 4471 | C | LYS | I | 143 | −23.988 | 27.258 | −17.793 | 1.00 | 27.22 | C |
| ATOM | 4472 | O | LYS | I | 143 | −24.610 | 28.108 | −17.139 | 1.00 | 27.19 | O |
| ATOM | 4473 | N | ASP | I | 144 | −23.286 | 27.529 | −18.899 | 1.00 | 26.86 | N |
| ATOM | 4474 | CA | ASP | I | 144 | −23.261 | 28.833 | −19.583 | 1.00 | 26.62 | C |
| ATOM | 4475 | CB | ASP | I | 144 | −24.577 | 29.091 | −20.340 | 1.00 | 26.80 | C |
| ATOM | 4476 | CG | ASP | I | 144 | −24.808 | 28.099 | −21.469 | 1.00 | 27.48 | C |
| ATOM | 4477 | OD1 | ASP | I | 144 | −23.831 | 27.570 | −22.061 | 1.00 | 27.00 | O |
| ATOM | 4478 | OD2 | ASP | I | 144 | −25.989 | 27.836 | −21.754 | 1.00 | 29.36 | O |
| ATOM | 4479 | C | ASP | I | 144 | −22.904 | 30.001 | −18.668 | 1.00 | 26.33 | C |
| ATOM | 4480 | O | ASP | I | 144 | −23.763 | 30.790 | −18.258 | 1.00 | 26.00 | O |
| ATOM | 4481 | N | TYR | I | 145 | −21.622 | 30.087 | −18.339 | 1.00 | 26.22 | N |
| ATOM | 4482 | CA | TYR | I | 145 | −21.117 | 31.199 | −17.565 | 1.00 | 25.97 | C |
| ATOM | 4483 | CB | TYR | I | 145 | −20.979 | 30.836 | −16.077 | 1.00 | 26.10 | C |
| ATOM | 4484 | CG | TYR | I | 145 | −19.927 | 29.781 | −15.774 | 1.00 | 27.60 | C |
| ATOM | 4485 | CD1 | TYR | I | 145 | −20.257 | 28.423 | −15.736 | 1.00 | 27.97 | C |
| ATOM | 4486 | CE1 | TYR | I | 145 | −19.296 | 27.462 | −15.453 | 1.00 | 27.45 | C |
| ATOM | 4487 | CZ | TYR | I | 145 | −17.994 | 27.856 | −15.210 | 1.00 | 27.79 | C |
| ATOM | 4488 | OH | TYR | I | 145 | −17.022 | 26.920 | −14.933 | 1.00 | 26.59 | O |
| ATOM | 4489 | CE2 | TYR | I | 145 | −17.646 | 29.195 | −15.246 | 1.00 | 28.02 | C |
| ATOM | 4490 | CD2 | TYR | I | 145 | −18.602 | 30.144 | −15.517 | 1.00 | 27.43 | C |
| ATOM | 4491 | C | TYR | I | 145 | −19.801 | 31.695 | −18.146 | 1.00 | 25.58 | C |
| ATOM | 4492 | O | TYR | I | 145 | −19.082 | 30.958 | −18.831 | 1.00 | 25.31 | O |
| ATOM | 4493 | N | PHE | I | 146 | −19.514 | 32.961 | −17.869 | 1.00 | 25.22 | N |
| ATOM | 4494 | CA | PHE | I | 146 | −18.260 | 33.585 | −18.238 | 1.00 | 24.98 | C |
| ATOM | 4495 | CB | PHE | I | 146 | −18.312 | 34.081 | −19.678 | 1.00 | 24.31 | C |
| ATOM | 4496 | CG | PHE | I | 146 | −17.023 | 34.647 | −20.162 | 1.00 | 22.79 | C |
| ATOM | 4497 | CD1 | PHE | I | 146 | −16.750 | 36.014 | −20.026 | 1.00 | 21.24 | C |
| ATOM | 4498 | CE1 | PHE | I | 146 | −15.554 | 36.552 | −20.476 | 1.00 | 18.53 | C |
| ATOM | 4499 | CZ | PHE | I | 146 | −14.602 | 35.720 | −21.065 | 1.00 | 18.64 | C |
| ATOM | 4500 | CE2 | PHE | I | 146 | −14.857 | 34.364 | −21.210 | 1.00 | 19.89 | C |
| ATOM | 4501 | CD2 | PHE | I | 146 | −16.067 | 33.827 | −20.749 | 1.00 | 20.70 | C |
| ATOM | 4502 | C | PHE | I | 146 | −17.988 | 34.748 | −17.292 | 1.00 | 25.33 | C |
| ATOM | 4503 | O | PHE | I | 146 | −18.900 | 35.520 | −16.983 | 1.00 | 25.88 | O |
| ATOM | 4504 | N | PRO | I | 147 | −16.744 | 34.867 | −16.806 | 1.00 | 25.47 | N |
| ATOM | 4505 | CA | PRO | I | 147 | −15.633 | 33.945 | −17.039 | 1.00 | 25.33 | C |
| ATOM | 4506 | CB | PRO | I | 147 | −14.432 | 34.889 | −17.066 | 1.00 | 25.41 | C |
| ATOM | 4507 | CG | PRO | I | 147 | −14.795 | 35.967 | −16.054 | 1.00 | 25.31 | C |
| ATOM | 4508 | CD | PRO | I | 147 | −16.314 | 36.051 | −16.029 | 1.00 | 25.35 | C |
| ATOM | 4509 | C | PRO | I | 147 | −15.469 | 32.933 | −15.906 | 1.00 | 25.52 | C |
| ATOM | 4510 | O | PRO | I | 147 | −16.312 | 32.865 | −14.994 | 1.00 | 25.49 | O |
| ATOM | 4511 | N | GLU | I | 148 | −14.385 | 32.163 | −15.963 | 1.00 | 25.72 | N |
| ATOM | 4512 | CA | GLU | I | 148 | −13.924 | 31.387 | −14.814 | 1.00 | 26.49 | C |
| ATOM | 4513 | CB | GLU | I | 148 | −12.744 | 30.506 | −15.226 | 1.00 | 26.57 | C |
| ATOM | 4514 | CG | GLU | I | 148 | −13.112 | 29.341 | −16.147 | 1.00 | 26.39 | C |
| ATOM | 4515 | CD | GLU | I | 148 | −13.393 | 28.043 | −15.393 | 1.00 | 26.09 | C |
| ATOM | 4516 | OE1 | GLU | I | 148 | −14.403 | 27.961 | −14.653 | 1.00 | 25.09 | O |
| ATOM | 4517 | OE2 | GLU | I | 148 | −12.602 | 27.090 | −15.565 | 1.00 | 26.06 | O |
| ATOM | 4518 | C | GLU | I | 148 | −13.504 | 32.362 | −13.697 | 1.00 | 26.91 | C |
| ATOM | 4519 | O | GLU | I | 148 | −13.136 | 33.498 | −13.984 | 1.00 | 27.36 | O |
| ATOM | 4520 | N | PRO | I | 149 | −13.559 | 31.931 | −12.423 | 1.00 | 27.37 | N |
| ATOM | 4521 | CA | PRO | I | 149 | −14.003 | 30.614 | −11.963 | 1.00 | 27.51 | C |
| ATOM | 4522 | CB | PRO | I | 149 | −12.945 | 30.248 | −10.906 | 1.00 | 27.45 | C |
| ATOM | 4523 | CG | PRO | I | 149 | −12.477 | 31.598 | −10.337 | 1.00 | 26.90 | C |
| ATOM | 4524 | CD | PRO | I | 149 | −12.884 | 32.683 | −11.340 | 1.00 | 27.35 | C |
| ATOM | 4525 | C | PRO | I | 149 | −15.376 | 30.625 | −11.311 | 1.00 | 27.76 | C |
| ATOM | 4526 | O | PRO | I | 149 | −15.888 | 31.686 | −10.941 | 1.00 | 27.92 | O |
| ATOM | 4527 | N | VAL | I | 150 | −15.963 | 29.436 | −11.195 | 1.00 | 27.94 | N |
| ATOM | 4528 | CA | VAL | I | 150 | −17.038 | 29.195 | −10.246 | 1.00 | 27.92 | C |
| ATOM | 4529 | CB | VAL | I | 150 | −18.278 | 28.523 | −10.891 | 1.00 | 28.04 | C |
| ATOM | 4530 | CG1 | VAL | I | 150 | −19.000 | 29.483 | −11.822 | 1.00 | 27.64 | C |
| ATOM | 4531 | CG2 | VAL | I | 150 | −17.884 | 27.227 | −11.605 | 1.00 | 27.59 | C |
| ATOM | 4532 | C | VAL | I | 150 | −16.509 | 28.263 | −9.164 | 1.00 | 27.95 | C |
| ATOM | 4533 | O | VAL | I | 150 | −15.571 | 27.494 | −9.398 | 1.00 | 28.00 | O |
| ATOM | 4534 | N | THR | I | 151 | −17.121 | 28.331 | −7.987 | 1.00 | 27.55 | N |
| ATOM | 4535 | CA | THR | I | 151 | −16.840 | 27.383 | −6.934 | 1.00 | 27.08 | C |
| ATOM | 4536 | CB | THR | I | 151 | −16.302 | 28.065 | −5.660 | 1.00 | 27.51 | C |
| ATOM | 4537 | OG1 | THR | I | 151 | −17.299 | 28.958 | −5.142 | 1.00 | 26.73 | O |
| ATOM | 4538 | CG2 | THR | I | 151 | −14.993 | 28.813 | −5.936 | 1.00 | 27.23 | C |
| ATOM | 4539 | C | THR | I | 151 | −18.137 | 26.706 | −6.567 | 1.00 | 26.70 | C |
| ATOM | 4540 | O | THR | I | 151 | −19.156 | 27.369 | −6.366 | 1.00 | 26.78 | O |
| ATOM | 4541 | N | VAL | I | 152 | −18.082 | 25.385 | −6.455 | 1.00 | 26.07 | N |
| ATOM | 4542 | CA | VAL | I | 152 | −19.219 | 24.583 | −6.017 | 1.00 | 24.89 | C |
| ATOM | 4543 | CB | VAL | I | 152 | −19.560 | 23.462 | −7.040 | 1.00 | 24.81 | C |
| ATOM | 4544 | CG1 | VAL | I | 152 | −20.765 | 22.642 | −6.580 | 1.00 | 23.61 | C |
| ATOM | 4545 | CG2 | VAL | I | 152 | −19.803 | 24.045 | −8.418 | 1.00 | 24.80 | C |
| ATOM | 4546 | C | VAL | I | 152 | −18.875 | 23.937 | −4.681 | 1.00 | 24.44 | C |
| ATOM | 4547 | O | VAL | I | 152 | −17.790 | 23.384 | −4.516 | 1.00 | 23.81 | O |
| ATOM | 4548 | N | SER | I | 153 | −19.800 | 24.016 | −3.734 | 1.00 | 24.10 | N |
| ATOM | 4549 | CA | SER | I | 153 | −19.690 | 23.242 | −2.502 | 1.00 | 23.97 | C |
| ATOM | 4550 | CB | SER | I | 153 | −19.360 | 24.152 | −1.321 | 1.00 | 23.61 | C |
| ATOM | 4551 | OG | SER | I | 153 | −20.466 | 24.949 | −0.969 | 1.00 | 23.35 | O |
| ATOM | 4552 | C | SER | I | 153 | −21.001 | 22.491 | −2.271 | 1.00 | 23.91 | C |
| ATOM | 4553 | O | SER | I | 153 | −22.028 | 22.847 | −2.853 | 1.00 | 24.08 | O |
| ATOM | 4554 | N | TRP | I | 154 | −20.961 | 21.455 | −1.437 | 1.00 | 23.57 | N |
| ATOM | 4555 | CA | TRP | I | 154 | −22.141 | 20.643 | −1.156 | 1.00 | 23.23 | C |
| ATOM | 4556 | CB | TRP | I | 154 | −21.907 | 19.192 | −1.570 | 1.00 | 22.59 | C |
| ATOM | 4557 | CG | TRP | I | 154 | −21.958 | 18.992 | −3.045 | 1.00 | 21.15 | C |
| ATOM | 4558 | CD1 | TRP | I | 154 | −20.912 | 19.054 | −3.916 | 1.00 | 20.35 | C |
| ATOM | 4559 | NE1 | TRP | I | 154 | −21.344 | 18.829 | −5.199 | 1.00 | 19.07 | N |
| ATOM | 4560 | CE2 | TRP | I | 154 | −22.695 | 18.606 | −5.176 | 1.00 | 19.91 | C |
| ATOM | 4561 | CD2 | TRP | I | 154 | −23.118 | 18.706 | −3.834 | 1.00 | 20.57 | C |
| ATOM | 4562 | CE3 | TRP | I | 154 | −24.476 | 18.507 | −3.533 | 1.00 | 20.15 | C |
| ATOM | 4563 | CZ3 | TRP | I | 154 | −25.354 | 18.231 | −4.570 | 1.00 | 18.50 | C |
| ATOM | 4564 | CH2 | TRP | I | 154 | −24.903 | 18.141 | −5.901 | 1.00 | 19.22 | C |
| ATOM | 4565 | CZ2 | TRP | I | 154 | −23.583 | 18.315 | −6.224 | 1.00 | 20.24 | C |
| ATOM | 4566 | C | TRP | I | 154 | −22.536 | 20.735 | 0.313 | 1.00 | 23.82 | C |
| ATOM | 4567 | O | TRP | I | 154 | −21.704 | 20.509 | 1.200 | 1.00 | 23.94 | O |
| ATOM | 4568 | N | ASN | I | 155 | −23.814 | 21.057 | 0.547 | 1.00 | 24.33 | N |
| ATOM | 4569 | CA | ASN | I | 155 | −24.369 | 21.350 | 1.876 | 1.00 | 24.98 | C |
| ATOM | 4570 | CB | ASN | I | 155 | −24.589 | 20.072 | 2.681 | 1.00 | 24.93 | C |
| ATOM | 4571 | CG | ASN | I | 155 | −25.571 | 19.129 | 2.004 | 1.00 | 26.11 | C |
| ATOM | 4572 | OD1 | ASN | I | 155 | −26.249 | 19.511 | 1.044 | 1.00 | 27.03 | O |
| ATOM | 4573 | ND2 | ASN | I | 155 | −25.657 | 17.892 | 2.498 | 1.00 | 25.73 | N |
| ATOM | 4574 | C | ASN | I | 155 | −23.555 | 22.370 | 2.657 | 1.00 | 25.52 | C |
| ATOM | 4575 | O | ASN | I | 155 | −23.236 | 22.162 | 3.830 | 1.00 | 26.14 | O |
| ATOM | 4576 | N | SER | I | 156 | −23.228 | 23.469 | 1.974 | 1.00 | 25.60 | N |
| ATOM | 4577 | CA | SER | I | 156 | −22.443 | 24.587 | 2.513 | 1.00 | 25.91 | C |
| ATOM | 4578 | CB | SER | I | 156 | −23.189 | 25.285 | 3.657 | 1.00 | 25.85 | C |
| ATOM | 4579 | OG | SER | I | 156 | −24.572 | 25.383 | 3.372 | 1.00 | 27.22 | O |
| ATOM | 4580 | C | SER | I | 156 | −21.029 | 24.195 | 2.967 | 1.00 | 25.76 | C |
| ATOM | 4581 | O | SER | I | 156 | −20.407 | 24.915 | 3.760 | 1.00 | 25.39 | O |
| ATOM | 4582 | N | GLY | I | 157 | −20.539 | 23.059 | 2.471 | 1.00 | 25.40 | N |
| ATOM | 4583 | CA | GLY | I | 157 | −19.194 | 22.585 | 2.799 | 1.00 | 24.94 | C |
| ATOM | 4584 | C | GLY | I | 157 | −19.196 | 21.444 | 3.797 | 1.00 | 24.78 | C |
| ATOM | 4585 | O | GLY | I | 157 | −18.138 | 20.915 | 4.155 | 1.00 | 25.27 | O |
| ATOM | 4586 | N | ALA | I | 158 | −20.381 | 21.046 | 4.247 | 1.00 | 24.34 | N |
| ATOM | 4587 | CA | ALA | I | 158 | −20.475 | 19.995 | 5.253 | 1.00 | 23.74 | C |
| ATOM | 4588 | CB | ALA | I | 158 | −21.839 | 20.013 | 5.945 | 1.00 | 23.33 | C |
| ATOM | 4589 | C | ALA | I | 158 | −20.191 | 18.628 | 4.653 | 1.00 | 23.35 | C |
| ATOM | 4590 | O | ALA | I | 158 | −19.915 | 17.672 | 5.386 | 1.00 | 23.51 | O |
| ATOM | 4591 | N | LEU | I | 159 | −20.253 | 18.531 | 3.327 | 1.00 | 22.68 | N |
| ATOM | 4592 | CA | LEU | I | 159 | −20.068 | 17.243 | 2.662 | 1.00 | 22.63 | C |
| ATOM | 4593 | CB | LEU | I | 159 | −21.383 | 16.778 | 2.015 | 1.00 | 22.73 | C |
| ATOM | 4594 | CG | LEU | I | 159 | −21.431 | 15.494 | 1.173 | 1.00 | 23.29 | C |
| ATOM | 4595 | CD1 | LEU | I | 159 | −21.268 | 14.239 | 2.015 | 1.00 | 23.24 | C |
| ATOM | 4596 | CD2 | LEU | I | 159 | −22.744 | 15.437 | 0.390 | 1.00 | 23.11 | C |
| ATOM | 4597 | C | LEU | I | 159 | −18.960 | 17.340 | 1.641 | 1.00 | 22.32 | C |
| ATOM | 4598 | O | LEU | I | 159 | −19.062 | 18.108 | 0.684 | 1.00 | 22.61 | O |
| ATOM | 4599 | N | THR | I | 160 | −17.894 | 16.574 | 1.841 | 1.00 | 22.11 | N |
| ATOM | 4600 | CA | THR | I | 160 | −16.777 | 16.611 | 0.903 | 1.00 | 22.09 | C |
| ATOM | 4601 | CB | THR | I | 160 | −15.509 | 17.284 | 1.498 | 1.00 | 22.03 | C |
| ATOM | 4602 | OG1 | THR | I | 160 | −15.040 | 16.538 | 2.628 | 1.00 | 22.39 | O |
| ATOM | 4603 | CG2 | THR | I | 160 | −15.805 | 18.702 | 1.926 | 1.00 | 20.59 | C |
| ATOM | 4604 | C | THR | I | 160 | −16.436 | 15.230 | 0.389 | 1.00 | 22.75 | C |
| ATOM | 4605 | O | THR | I | 160 | −15.834 | 15.104 | −0.681 | 1.00 | 23.04 | O |
| ATOM | 4606 | N | SER | I | 161 | −16.826 | 14.195 | 1.138 | 1.00 | 23.17 | N |
| ATOM | 4607 | CA | SER | I | 161 | −16.557 | 12.804 | 0.734 | 1.00 | 23.67 | C |
| ATOM | 4608 | CB | SER | I | 161 | −16.956 | 11.812 | 1.834 | 1.00 | 23.56 | C |
| ATOM | 4609 | OG | SER | I | 161 | −16.178 | 12.005 | 2.992 | 1.00 | 24.53 | O |
| ATOM | 4610 | C | SER | I | 161 | −17.337 | 12.476 | −0.516 | 1.00 | 23.60 | C |
| ATOM | 4611 | O | SER | I | 161 | −18.523 | 12.788 | −0.587 | 1.00 | 23.84 | O |
| ATOM | 4612 | N | GLY | I | 162 | −16.679 | 11.855 | −1.493 | 1.00 | 23.70 | N |
| ATOM | 4613 | CA | GLY | I | 162 | −17.349 | 11.433 | −2.724 | 1.00 | 23.91 | C |
| ATOM | 4614 | C | GLY | I | 162 | −17.679 | 12.571 | −3.689 | 1.00 | 24.45 | C |
| ATOM | 4615 | O | GLY | I | 162 | −18.286 | 12.345 | −4.740 | 1.00 | 24.91 | O |
| ATOM | 4616 | N | VAL | I | 163 | −17.269 | 13.791 | −3.355 | 1.00 | 24.01 | N |
| ATOM | 4617 | CA | VAL | I | 163 | −17.478 | 14.914 | −4.247 | 1.00 | 23.80 | C |
| ATOM | 4618 | CB | VAL | I | 163 | −17.525 | 16.250 | −3.483 | 1.00 | 23.54 | C |
| ATOM | 4619 | CG1 | VAL | I | 163 | −17.554 | 17.429 | −4.446 | 1.00 | 22.59 | C |
| ATOM | 4620 | CG2 | VAL | I | 163 | −18.742 | 16.283 | −2.583 | 1.00 | 22.93 | C |
| ATOM | 4621 | C | VAL | I | 163 | −16.411 | 14.955 | −5.329 | 1.00 | 24.21 | C |
| ATOM | 4622 | O | VAL | I | 163 | −15.217 | 14.964 | −5.037 | 1.00 | 24.80 | O |
| ATOM | 4623 | N | HIS | I | 164 | −16.838 | 14.966 | −6.582 | 1.00 | 24.16 | N |
| ATOM | 4624 | CA | HIS | I | 164 | −15.918 | 15.286 | −7.650 | 1.00 | 24.87 | C |
| ATOM | 4625 | CB | HIS | I | 164 | −15.751 | 14.125 | −8.631 | 1.00 | 24.97 | C |
| ATOM | 4626 | CG | HIS | I | 164 | −15.132 | 12.893 | −8.035 | 1.00 | 27.14 | C |
| ATOM | 4627 | ND1 | HIS | I | 164 | −13.811 | 12.836 | −7.627 | 1.00 | 29.07 | N |
| ATOM | 4628 | CE1 | HIS | I | 164 | −13.542 | 11.621 | −7.177 | 1.00 | 28.72 | C |
| ATOM | 4629 | NE2 | HIS | I | 164 | −14.638 | 10.886 | −7.279 | 1.00 | 28.05 | N |
| ATOM | 4630 | CD2 | HIS | I | 164 | −15.643 | 11.655 | −7.820 | 1.00 | 27.41 | C |
| ATOM | 4631 | C | HIS | I | 164 | −16.451 | 16.515 | −8.369 | 1.00 | 24.86 | C |
| ATOM | 4632 | O | HIS | I | 164 | −17.562 | 16.494 | −8.892 | 1.00 | 25.57 | O |
| ATOM | 4633 | N | THR | I | 165 | −15.678 | 17.595 | −8.369 | 1.00 | 24.30 | N |
| ATOM | 4634 | CA | THR | I | 165 | −16.039 | 18.763 | −9.143 | 1.00 | 23.62 | C |
| ATOM | 4635 | CB | THR | I | 165 | −16.045 | 20.054 | −8.298 | 1.00 | 23.86 | C |
| ATOM | 4636 | OG1 | THR | I | 165 | −17.053 | 19.945 | −7.281 | 1.00 | 23.17 | O |
| ATOM | 4637 | CG2 | THR | I | 165 | −16.345 | 21.289 | −9.176 | 1.00 | 23.53 | C |
| ATOM | 4638 | C | THR | I | 165 | −15.069 | 18.838 | −10.304 | 1.00 | 23.79 | C |
| ATOM | 4639 | O | THR | I | 165 | −13.846 | 18.856 | −10.132 | 1.00 | 23.52 | O |
| ATOM | 4640 | N | PHE | I | 166 | −15.636 | 18.857 | −11.502 | 1.00 | 23.82 | N |
| ATOM | 4641 | CA | PHE | I | 166 | −14.862 | 18.649 | −12.704 | 1.00 | 23.54 | C |
| ATOM | 4642 | CB | PHE | I | 166 | −15.722 | 17.941 | −13.751 | 1.00 | 23.29 | C |
| ATOM | 4643 | CG | PHE | I | 166 | −16.009 | 16.512 | −13.415 | 1.00 | 22.08 | C |
| ATOM | 4644 | CD1 | PHE | I | 166 | −15.039 | 15.530 | −13.610 | 1.00 | 21.93 | C |
| ATOM | 4645 | CE1 | PHE | I | 166 | −15.304 | 14.193 | −13.287 | 1.00 | 20.99 | C |
| ATOM | 4646 | CZ | PHE | I | 166 | −16.545 | 13.846 | −12.772 | 1.00 | 19.33 | C |
| ATOM | 4647 | CE2 | PHE | I | 166 | −17.508 | 14.819 | −12.576 | 1.00 | 19.25 | C |
| ATOM | 4648 | CD2 | PHE | I | 166 | −17.238 | 16.142 | −12.898 | 1.00 | 20.45 | C |
| ATOM | 4649 | C | PHE | I | 166 | −14.317 | 19.948 | −13.234 | 1.00 | 23.67 | C |
| ATOM | 4650 | O | PHE | I | 166 | −14.860 | 21.002 | −12.938 | 1.00 | 23.84 | O |
| ATOM | 4651 | N | PRO | I | 167 | −13.215 | 19.882 | −13.995 | 1.00 | 24.18 | N |
| ATOM | 4652 | CA | PRO | I | 167 | −12.759 | 21.069 | −14.704 | 1.00 | 24.43 | C |
| ATOM | 4653 | CB | PRO | I | 167 | −11.547 | 20.576 | −15.503 | 1.00 | 24.29 | C |
| ATOM | 4654 | CG | PRO | I | 167 | −11.091 | 19.358 | −14.844 | 1.00 | 24.43 | C |
| ATOM | 4655 | CD | PRO | I | 167 | −12.258 | 18.763 | −14.093 | 1.00 | 24.42 | C |
| ATOM | 4656 | C | PRO | I | 167 | −13.841 | 21.559 | −15.667 | 1.00 | 24.91 | C |
| ATOM | 4657 | O | PRO | I | 167 | −14.611 | 20.756 | −16.206 | 1.00 | 24.87 | O |
| ATOM | 4658 | N | ALA | I | 168 | −13.900 | 22.872 | −15.862 | 1.00 | 25.11 | N |
| ATOM | 4659 | CA | ALA | I | 168 | −14.833 | 23.462 | −16.805 | 1.00 | 25.58 | C |
| ATOM | 4660 | CB | ALA | I | 168 | −14.865 | 24.979 | −16.644 | 1.00 | 25.64 | C |
| ATOM | 4661 | C | ALA | I | 168 | −14.459 | 23.094 | −18.235 | 1.00 | 25.63 | C |
| ATOM | 4662 | O | ALA | I | 168 | −13.311 | 22.758 | −18.524 | 1.00 | 26.06 | O |
| ATOM | 4663 | N | VAL | I | 169 | −15.438 | 23.142 | −19.126 | 1.00 | 25.52 | N |
| ATOM | 4664 | CA | VAL | I | 169 | −15.142 | 23.043 | −20.546 | 1.00 | 25.36 | C |
| ATOM | 4665 | CB | VAL | I | 169 | −15.765 | 21.783 | −21.203 | 1.00 | 25.61 | C |
| ATOM | 4666 | CG1 | VAL | I | 169 | −14.925 | 20.541 | −20.872 | 1.00 | 25.17 | C |
| ATOM | 4667 | CG2 | VAL | I | 169 | −17.233 | 21.582 | −20.756 | 1.00 | 26.02 | C |
| ATOM | 4668 | C | VAL | I | 169 | −15.604 | 24.316 | −21.219 | 1.00 | 24.98 | C |
| ATOM | 4669 | O | VAL | I | 169 | −16.667 | 24.850 | −20.895 | 1.00 | 24.82 | O |
| ATOM | 4670 | N | LEU | I | 170 | −14.766 | 24.830 | −22.109 | 1.00 | 24.80 | N |
| ATOM | 4671 | CA | LEU | I | 170 | −15.146 | 25.935 | −22.955 | 1.00 | 24.74 | C |
| ATOM | 4672 | CB | LEU | I | 170 | −13.904 | 26.641 | −23.505 | 1.00 | 24.21 | C |
| ATOM | 4673 | CG | LEU | I | 170 | −14.129 | 27.892 | −24.369 | 1.00 | 23.47 | C |
| ATOM | 4674 | CD1 | LEU | I | 170 | −14.783 | 29.035 | −23.595 | 1.00 | 21.58 | C |
| ATOM | 4675 | CD2 | LEU | I | 170 | −12.821 | 28.354 | −24.965 | 1.00 | 22.89 | C |
| ATOM | 4676 | C | LEU | I | 170 | −15.983 | 25.378 | −24.093 | 1.00 | 25.25 | C |
| ATOM | 4677 | O | LEU | I | 170 | −15.476 | 24.635 | −24.926 | 1.00 | 25.49 | O |
| ATOM | 4678 | N | GLN | I | 171 | −17.263 | 25.726 | −24.118 | 1.00 | 26.03 | N |
| ATOM | 4679 | CA | GLN | I | 171 | −18.136 | 25.405 | −25.254 | 1.00 | 26.85 | C |
| ATOM | 4680 | CB | GLN | I | 171 | −19.609 | 25.538 | −24.856 | 1.00 | 26.70 | C |
| ATOM | 4681 | CG | GLN | I | 171 | −19.991 | 24.741 | −23.600 | 1.00 | 26.74 | C |
| ATOM | 4682 | CD | GLN | I | 171 | −21.207 | 25.299 | −22.897 | 1.00 | 26.97 | C |
| ATOM | 4683 | OE1 | GLN | I | 171 | −22.303 | 24.750 | −22.998 | 1.00 | 26.25 | O |
| ATOM | 4684 | NE2 | GLN | I | 171 | −21.022 | 26.407 | −22.182 | 1.00 | 27.11 | N |
| ATOM | 4685 | C | GLN | I | 171 | −17.834 | 26.305 | −26.465 | 1.00 | 27.65 | C |
| ATOM | 4686 | O | GLN | I | 171 | −17.288 | 27.409 | −26.323 | 1.00 | 27.54 | O |
| ATOM | 4687 | N | SER | I | 172 | −18.207 | 25.821 | −27.651 | 1.00 | 28.53 | N |
| ATOM | 4688 | CA | SER | I | 172 | −17.991 | 26.530 | −28.919 | 1.00 | 29.13 | C |
| ATOM | 4689 | CB | SER | I | 172 | −18.495 | 25.677 | −30.078 | 1.00 | 29.36 | C |
| ATOM | 4690 | OG | SER | I | 172 | −19.876 | 25.393 | −29.910 | 1.00 | 30.22 | O |
| ATOM | 4691 | C | SER | I | 172 | −18.662 | 27.906 | −28.952 | 1.00 | 29.30 | C |
| ATOM | 4692 | O | SER | I | 172 | −18.284 | 28.772 | −29.754 | 1.00 | 29.77 | O |
| ATOM | 4693 | N | SER | I | 173 | −19.646 | 28.094 | −28.072 | 1.00 | 29.00 | N |
| ATOM | 4694 | CA | SER | I | 173 | −20.318 | 29.373 | −27.878 | 1.00 | 28.91 | C |
| ATOM | 4695 | CB | SER | I | 173 | −21.638 | 29.158 | −27.130 | 1.00 | 28.89 | C |
| ATOM | 4696 | OG | SER | I | 173 | −21.391 | 28.825 | −25.781 | 1.00 | 29.04 | O |
| ATOM | 4697 | C | SER | I | 173 | −19.458 | 30.387 | −27.110 | 1.00 | 28.90 | C |
| ATOM | 4698 | O | SER | I | 173 | −19.801 | 31.576 | −27.027 | 1.00 | 28.63 | O |
| ATOM | 4699 | N | GLY | I | 174 | −18.358 | 29.908 | −26.529 | 1.00 | 28.75 | N |
| ATOM | 4700 | CA | GLY | I | 174 | −17.444 | 30.765 | −25.775 | 1.00 | 28.05 | C |
| ATOM | 4701 | C | GLY | I | 174 | −17.858 | 30.944 | −24.325 | 1.00 | 27.47 | C |
| ATOM | 4702 | O | GLY | I | 174 | −17.262 | 31.741 | −23.597 | 1.00 | 27.27 | O |
| ATOM | 4703 | N | LEU | I | 175 | −18.884 | 30.206 | −23.910 | 1.00 | 26.80 | N |
| ATOM | 4704 | CA | LEU | I | 175 | −19.266 | 30.141 | −22.506 | 1.00 | 26.41 | C |
| ATOM | 4705 | CB | LEU | I | 175 | −20.783 | 30.272 | −22.351 | 1.00 | 26.24 | C |
| ATOM | 4706 | CG | LEU | I | 175 | −21.413 | 31.526 | −22.992 | 1.00 | 26.69 | C |
| ATOM | 4707 | CD1 | LEU | I | 175 | −22.928 | 31.380 | −23.130 | 1.00 | 25.08 | C |
| ATOM | 4708 | CD2 | LEU | I | 175 | −21.038 | 32.813 | −22.238 | 1.00 | 23.93 | C |
| ATOM | 4709 | C | LEU | I | 175 | −18.785 | 28.819 | −21.913 | 1.00 | 26.41 | C |
| ATOM | 4710 | O | LEU | I | 175 | −18.633 | 27.814 | −22.636 | 1.00 | 26.04 | O |
| ATOM | 4711 | N | TYR | I | 176 | −18.546 | 28.824 | −20.599 | 1.00 | 25.99 | N |
| ATOM | 4712 | CA | TYR | I | 176 | −18.079 | 27.631 | −19.893 | 1.00 | 25.28 | C |
| ATOM | 4713 | CB | TYR | I | 176 | −17.133 | 28.003 | −18.745 | 1.00 | 25.00 | C |
| ATOM | 4714 | CG | TYR | I | 176 | −15.820 | 28.582 | −19.183 | 1.00 | 24.15 | C |
| ATOM | 4715 | CD1 | TYR | I | 176 | −14.713 | 27.755 | −19.420 | 1.00 | 22.86 | C |
| ATOM | 4716 | CE1 | TYR | I | 176 | −13.498 | 28.286 | −19.826 | 1.00 | 22.93 | C |
| ATOM | 4717 | CZ | TYR | I | 176 | −13.375 | 29.665 | −19.996 | 1.00 | 24.04 | C |
| ATOM | 4718 | OH | TYR | I | 176 | −12.177 | 30.205 | −20.390 | 1.00 | 25.00 | O |
| ATOM | 4719 | CE2 | TYR | I | 176 | −14.454 | 30.504 | −19.768 | 1.00 | 23.38 | C |
| ATOM | 4720 | CD2 | TYR | I | 176 | −15.672 | 29.955 | −19.355 | 1.00 | 23.80 | C |
| ATOM | 4721 | C | TYR | I | 176 | −19.231 | 26.798 | −19.346 | 1.00 | 25.24 | C |
| ATOM | 4722 | O | TYR | I | 176 | −20.315 | 27.308 | −19.067 | 1.00 | 24.78 | O |
| ATOM | 4723 | N | SER | I | 177 | −18.957 | 25.510 | −19.173 | 1.00 | 25.35 | N |
| ATOM | 4724 | CA | SER | I | 177 | −19.867 | 24.586 | −18.520 | 1.00 | 25.23 | C |
| ATOM | 4725 | CB | SER | I | 177 | −20.631 | 23.804 | −19.578 | 1.00 | 25.03 | C |
| ATOM | 4726 | OG | SER | I | 177 | −21.712 | 23.098 | −18.998 | 1.00 | 27.06 | O |
| ATOM | 4727 | C | SER | I | 177 | −19.073 | 23.639 | −17.606 | 1.00 | 24.98 | C |
| ATOM | 4728 | O | SER | I | 177 | −17.978 | 23.193 | −17.968 | 1.00 | 25.15 | O |
| ATOM | 4729 | N | LEU | I | 178 | −19.604 | 23.356 | −16.418 | 1.00 | 24.86 | N |
| ATOM | 4730 | CA | LEU | I | 178 | −18.984 | 22.385 | −15.515 | 1.00 | 24.84 | C |
| ATOM | 4731 | CB | LEU | I | 178 | −17.917 | 23.022 | −14.591 | 1.00 | 24.74 | C |
| ATOM | 4732 | CG | LEU | I | 178 | −18.109 | 23.665 | −13.205 | 1.00 | 24.20 | C |
| ATOM | 4733 | CD1 | LEU | I | 178 | −18.839 | 22.819 | −12.172 | 1.00 | 22.35 | C |
| ATOM | 4734 | CD2 | LEU | I | 178 | −16.741 | 24.062 | −12.644 | 1.00 | 23.39 | C |
| ATOM | 4735 | C | LEU | I | 178 | −19.974 | 21.599 | −14.692 | 1.00 | 24.96 | C |
| ATOM | 4736 | O | LEU | I | 178 | −21.104 | 22.052 | −14.440 | 1.00 | 24.96 | O |
| ATOM | 4737 | N | SER | I | 179 | −19.500 | 20.433 | −14.247 | 1.00 | 25.16 | N |
| ATOM | 4738 | CA | SER | I | 179 | −20.256 | 19.504 | −13.419 | 1.00 | 25.25 | C |
| ATOM | 4739 | CB | SER | I | 179 | −20.407 | 18.180 | −14.145 | 1.00 | 25.20 | C |
| ATOM | 4740 | OG | SER | I | 179 | −21.534 | 18.244 | −14.988 | 1.00 | 27.76 | O |
| ATOM | 4741 | C | SER | I | 179 | −19.616 | 19.236 | −12.072 | 1.00 | 25.18 | C |
| ATOM | 4742 | O | SER | I | 179 | −18.384 | 19.212 | −11.941 | 1.00 | 24.58 | O |
| ATOM | 4743 | N | SER | I | 180 | −20.477 | 19.027 | −11.079 | 1.00 | 25.44 | N |
| ATOM | 4744 | CA | SER | I | 180 | −20.065 | 18.551 | −9.774 | 1.00 | 26.02 | C |
| ATOM | 4745 | CB | SER | I | 180 | −20.094 | 19.674 | −8.736 | 1.00 | 25.98 | C |
| ATOM | 4746 | OG | SER | I | 180 | −19.537 | 19.248 | −7.499 | 1.00 | 26.23 | O |
| ATOM | 4747 | C | SER | I | 180 | −20.992 | 17.422 | −9.364 | 1.00 | 26.48 | C |
| ATOM | 4748 | O | SER | I | 180 | −22.215 | 17.548 | −9.455 | 1.00 | 26.55 | O |
| ATOM | 4749 | N | VAL | I | 181 | −20.396 | 16.314 | −8.936 | 1.00 | 26.99 | N |
| ATOM | 4750 | CA | VAL | I | 181 | −21.143 | 15.131 | −8.533 | 1.00 | 27.61 | C |
| ATOM | 4751 | CB | VAL | I | 181 | −21.005 | 13.951 | −9.541 | 1.00 | 27.80 | C |
| ATOM | 4752 | CG1 | VAL | I | 181 | −21.513 | 14.338 | −10.939 | 1.00 | 26.51 | C |
| ATOM | 4753 | CG2 | VAL | I | 181 | −19.560 | 13.428 | −9.590 | 1.00 | 26.94 | C |
| ATOM | 4754 | C | VAL | I | 181 | −20.667 | 14.649 | −7.180 | 1.00 | 28.46 | C |
| ATOM | 4755 | O | VAL | I | 181 | −19.575 | 14.999 | −6.724 | 1.00 | 28.80 | O |
| ATOM | 4756 | N | VAL | I | 182 | −21.512 | 13.848 | −6.548 | 1.00 | 29.25 | N |
| ATOM | 4757 | CA | VAL | I | 182 | −21.203 | 13.182 | −5.302 | 1.00 | 29.90 | C |
| ATOM | 4758 | CB | VAL | I | 182 | −21.522 | 14.067 | −4.070 | 1.00 | 30.06 | C |
| ATOM | 4759 | CG1 | VAL | I | 182 | −22.900 | 14.729 | −4.190 | 1.00 | 29.51 | C |
| ATOM | 4760 | CG2 | VAL | I | 182 | −21.373 | 13.263 | −2.749 | 1.00 | 29.32 | C |
| ATOM | 4761 | C | VAL | I | 182 | −21.973 | 11.872 | −5.259 | 1.00 | 30.55 | C |
| ATOM | 4762 | O | VAL | I | 182 | −23.152 | 11.830 | −5.599 | 1.00 | 30.45 | O |
| ATOM | 4763 | N | THR | I | 183 | −21.286 | 10.797 | −4.885 | 1.00 | 31.59 | N |
| ATOM | 4764 | CA | THR | I | 183 | −21.937 | 9.511 | −4.652 | 1.00 | 32.38 | C |
| ATOM | 4765 | CB | THR | I | 183 | −21.039 | 8.322 | −5.008 | 1.00 | 32.26 | C |
| ATOM | 4766 | OG1 | THR | I | 183 | −19.757 | 8.476 | −4.380 | 1.00 | 32.66 | O |
| ATOM | 4767 | CG2 | THR | I | 183 | −20.855 | 8.225 | −6.511 | 1.00 | 31.44 | C |
| ATOM | 4768 | C | THR | I | 183 | −22.329 | 9.439 | −3.194 | 1.00 | 33.43 | C |
| ATOM | 4769 | O | THR | I | 183 | −21.535 | 9.769 | −2.323 | 1.00 | 33.38 | O |
| ATOM | 4770 | N | VAL | I | 184 | −23.573 | 9.040 | −2.942 | 1.00 | 35.09 | N |
| ATOM | 4771 | CA | VAL | I | 184 | −24.122 | 8.942 | −1.584 | 1.00 | 36.53 | C |
| ATOM | 4772 | CB | VAL | I | 184 | −25.144 | 10.081 | −1.298 | 1.00 | 36.75 | C |
| ATOM | 4773 | CG1 | VAL | I | 184 | −24.448 | 11.452 | −1.337 | 1.00 | 36.63 | C |
| ATOM | 4774 | CG2 | VAL | I | 184 | −26.336 | 10.014 | −2.260 | 1.00 | 36.21 | C |
| ATOM | 4775 | C | VAL | I | 184 | −24.778 | 7.573 | −1.337 | 1.00 | 37.54 | C |
| ATOM | 4776 | O | VAL | I | 184 | −25.068 | 6.853 | −2.291 | 1.00 | 37.57 | O |
| ATOM | 4777 | N | PRO | I | 185 | −25.012 | 7.212 | −0.059 | 1.00 | 38.54 | N |
| ATOM | 4778 | CA | PRO | I | 185 | −25.737 | 5.975 | 0.240 | 1.00 | 39.37 | C |
| ATOM | 4779 | CB | PRO | I | 185 | −25.701 | 5.897 | 1.770 | 1.00 | 39.42 | C |
| ATOM | 4780 | CG | PRO | I | 185 | −24.658 | 6.889 | 2.210 | 1.00 | 39.29 | C |
| ATOM | 4781 | CD | PRO | I | 185 | −24.649 | 7.946 | 1.167 | 1.00 | 38.79 | C |
| ATOM | 4782 | C | PRO | I | 185 | −27.179 | 6.087 | −0.212 | 1.00 | 40.19 | C |
| ATOM | 4783 | O | PRO | I | 185 | −27.872 | 7.024 | 0.196 | 1.00 | 40.34 | O |
| ATOM | 4784 | N | SER | I | 186 | −27.633 | 5.142 | −1.035 | 1.00 | 41.16 | N |
| ATOM | 4785 | CA | SER | I | 186 | −29.013 | 5.165 | −1.545 | 1.00 | 42.21 | C |
| ATOM | 4786 | CB | SER | I | 186 | −29.240 | 4.082 | −2.604 | 1.00 | 42.19 | C |
| ATOM | 4787 | OG | SER | I | 186 | −28.802 | 2.810 | −2.161 | 1.00 | 42.55 | O |
| ATOM | 4788 | C | SER | I | 186 | −30.078 | 5.097 | −0.442 | 1.00 | 42.89 | C |
| ATOM | 4789 | O | SER | I | 186 | −31.212 | 5.542 | −0.635 | 1.00 | 43.06 | O |
| ATOM | 4790 | N | SER | I | 187 | −29.700 | 4.569 | 0.718 | 1.00 | 43.65 | N |
| ATOM | 4791 | CA | SER | I | 187 | −30.598 | 4.547 | 1.878 | 1.00 | 44.67 | C |
| ATOM | 4792 | CB | SER | I | 187 | −30.073 | 3.591 | 2.962 | 1.00 | 44.74 | C |
| ATOM | 4793 | OG | SER | I | 187 | −28.680 | 3.749 | 3.182 | 1.00 | 45.57 | O |
| ATOM | 4794 | C | SER | I | 187 | −30.920 | 5.926 | 2.482 | 1.00 | 44.91 | C |
| ATOM | 4795 | O | SER | I | 187 | −31.908 | 6.065 | 3.199 | 1.00 | 45.37 | O |
| ATOM | 4796 | N | SER | I | 188 | −30.107 | 6.937 | 2.187 | 1.00 | 45.16 | N |
| ATOM | 4797 | CA | SER | I | 188 | −30.300 | 8.285 | 2.753 | 1.00 | 45.22 | C |
| ATOM | 4798 | CB | SER | I | 188 | −28.945 | 8.949 | 3.041 | 1.00 | 45.39 | C |
| ATOM | 4799 | OG | SER | I | 188 | −28.309 | 9.393 | 1.846 | 1.00 | 45.53 | O |
| ATOM | 4800 | C | SER | I | 188 | −31.155 | 9.220 | 1.888 | 1.00 | 45.23 | C |
| ATOM | 4801 | O | SER | I | 188 | −31.594 | 10.275 | 2.354 | 1.00 | 45.24 | O |
| ATOM | 4802 | N | LEU | I | 189 | −31.397 | 8.830 | 0.639 | 1.00 | 45.29 | N |
| ATOM | 4803 | CA | LEU | I | 189 | −32.140 | 9.663 | −0.315 | 1.00 | 45.61 | C |
| ATOM | 4804 | CB | LEU | I | 189 | −32.240 | 8.963 | −1.671 | 1.00 | 45.50 | C |
| ATOM | 4805 | CG | LEU | I | 189 | −30.956 | 8.789 | −2.480 | 1.00 | 45.28 | C |
| ATOM | 4806 | CD1 | LEU | I | 189 | −31.207 | 7.805 | −3.607 | 1.00 | 45.61 | C |
| ATOM | 4807 | CD2 | LEU | I | 189 | −30.452 | 10.124 | −3.020 | 1.00 | 44.96 | C |
| ATOM | 4808 | C | LEU | I | 189 | −33.539 | 10.119 | 0.135 | 1.00 | 45.89 | C |
| ATOM | 4809 | O | LEU | I | 189 | −34.089 | 11.078 | −0.413 | 1.00 | 46.11 | O |
| ATOM | 4810 | N | GLY | I | 190 | −34.117 | 9.425 | 1.110 | 1.00 | 46.05 | N |
| ATOM | 4811 | CA | GLY | I | 190 | −35.417 | 9.810 | 1.654 | 1.00 | 46.11 | C |
| ATOM | 4812 | C | GLY | I | 190 | −35.259 | 10.830 | 2.762 | 1.00 | 46.28 | C |
| ATOM | 4813 | O | GLY | I | 190 | −36.051 | 11.771 | 2.885 | 1.00 | 46.43 | O |
| ATOM | 4814 | N | THR | I | 191 | −34.222 | 10.646 | 3.573 | 1.00 | 46.18 | N |
| ATOM | 4815 | CA | THR | I | 191 | −33.960 | 11.553 | 4.675 | 1.00 | 45.78 | C |
| ATOM | 4816 | CB | THR | I | 191 | −33.433 | 10.800 | 5.923 | 1.00 | 46.05 | C |
| ATOM | 4817 | OG1 | THR | I | 191 | −34.044 | 9.503 | 5.992 | 1.00 | 46.11 | O |
| ATOM | 4818 | CG2 | THR | I | 191 | −33.761 | 11.576 | 7.205 | 1.00 | 46.31 | C |
| ATOM | 4819 | C | THR | I | 191 | −33.038 | 12.678 | 4.181 | 1.00 | 45.24 | C |
| ATOM | 4820 | O | THR | I | 191 | −33.540 | 13.642 | 3.599 | 1.00 | 45.40 | O |
| ATOM | 4821 | N | GLN | I | 192 | −31.719 | 12.547 | 4.368 | 1.00 | 44.37 | N |
| ATOM | 4822 | CA | GLN | I | 192 | −30.746 | 13.606 | 4.007 | 1.00 | 43.41 | C |
| ATOM | 4823 | CB | GLN | I | 192 | −29.311 | 13.061 | 3.899 | 1.00 | 43.74 | C |
| ATOM | 4824 | CG | GLN | I | 192 | −28.393 | 13.351 | 5.109 | 1.00 | 45.12 | C |
| ATOM | 4825 | CD | GLN | I | 192 | −28.176 | 14.842 | 5.411 | 1.00 | 46.51 | C |
| ATOM | 4826 | OE1 | GLN | I | 192 | −28.247 | 15.702 | 4.528 | 1.00 | 45.11 | O |
| ATOM | 4827 | NE2 | GLN | I | 192 | −27.899 | 15.144 | 6.683 | 1.00 | 47.98 | N |
| ATOM | 4828 | C | GLN | I | 192 | −31.062 | 14.407 | 2.743 | 1.00 | 42.26 | C |
| ATOM | 4829 | O | GLN | I | 192 | −31.224 | 13.845 | 1.656 | 1.00 | 41.99 | O |
| ATOM | 4830 | N | THR | I | 193 | −31.129 | 15.726 | 2.908 | 1.00 | 40.99 | N |
| ATOM | 4831 | CA | THR | I | 193 | −31.325 | 16.644 | 1.794 | 1.00 | 39.79 | C |
| ATOM | 4832 | CB | THR | I | 193 | −32.105 | 17.904 | 2.234 | 1.00 | 39.99 | C |
| ATOM | 4833 | OG1 | THR | I | 193 | −31.214 | 18.808 | 2.894 | 1.00 | 40.61 | O |
| ATOM | 4834 | CG2 | THR | I | 193 | −33.266 | 17.549 | 3.181 | 1.00 | 40.05 | C |
| ATOM | 4835 | C | THR | I | 193 | −29.965 | 17.063 | 1.216 | 1.00 | 38.69 | C |
| ATOM | 4836 | O | THR | I | 193 | −29.034 | 17.366 | 1.966 | 1.00 | 38.24 | O |
| ATOM | 4837 | N | TYR | I | 194 | −29.856 | 17.078 | −0.113 | 1.00 | 37.50 | N |
| ATOM | 4838 | CA | TYR | I | 194 | −28.602 | 17.458 | −0.777 | 1.00 | 36.33 | C |
| ATOM | 4839 | CB | TYR | I | 194 | −28.092 | 16.321 | −1.659 | 1.00 | 36.22 | C |
| ATOM | 4840 | CG | TYR | I | 194 | −27.738 | 15.102 | −0.854 | 1.00 | 36.20 | C |
| ATOM | 4841 | CD1 | TYR | I | 194 | −28.650 | 14.062 | −0.689 | 1.00 | 35.10 | C |
| ATOM | 4842 | CE1 | TYR | I | 194 | −28.332 | 12.951 | 0.068 | 1.00 | 35.73 | C |
| ATOM | 4843 | CZ | TYR | I | 194 | −27.091 | 12.877 | 0.679 | 1.00 | 36.54 | C |
| ATOM | 4844 | OH | TYR | I | 194 | −26.760 | 11.783 | 1.431 | 1.00 | 37.83 | O |
| ATOM | 4845 | CE2 | TYR | I | 194 | −26.171 | 13.896 | 0.537 | 1.00 | 35.95 | C |
| ATOM | 4846 | CD2 | TYR | I | 194 | −26.497 | 15.002 | −0.226 | 1.00 | 35.92 | C |
| ATOM | 4847 | C | TYR | I | 194 | −28.709 | 18.759 | −1.566 | 1.00 | 35.48 | C |
| ATOM | 4848 | O | TYR | I | 194 | −29.618 | 18.923 | −2.375 | 1.00 | 35.35 | O |
| ATOM | 4849 | N | ILE | I | 195 | −27.775 | 19.677 | −1.321 | 1.00 | 34.47 | N |
| ATOM | 4850 | CA | ILE | I | 195 | −27.805 | 21.004 | −1.939 | 1.00 | 33.73 | C |
| ATOM | 4851 | CB | ILE | I | 195 | −28.251 | 22.096 | −0.930 | 1.00 | 33.63 | C |
| ATOM | 4852 | CG1 | ILE | I | 195 | −29.662 | 21.806 | −0.393 | 1.00 | 33.44 | C |
| ATOM | 4853 | CD1 | ILE | I | 195 | −29.987 | 22.472 | 0.934 | 1.00 | 31.47 | C |
| ATOM | 4854 | CG2 | ILE | I | 195 | −28.202 | 23.483 | −1.577 | 1.00 | 33.70 | C |
| ATOM | 4855 | C | ILE | I | 195 | −26.446 | 21.387 | −2.515 | 1.00 | 33.38 | C |
| ATOM | 4856 | O | ILE | I | 195 | −25.439 | 21.383 | −1.815 | 1.00 | 33.33 | O |
| ATOM | 4857 | N | CYS | I | 196 | −26.411 | 21.731 | −3.793 | 1.00 | 32.99 | N |
| ATOM | 4858 | CA | CYS | I | 196 | −25.181 | 22.257 | −4.333 | 1.00 | 32.95 | C |
| ATOM | 4859 | CB | CYS | I | 196 | −24.911 | 21.738 | −5.753 | 1.00 | 32.69 | C |
| ATOM | 4860 | SG | CYS | I | 196 | −25.870 | 22.499 | −7.044 | 1.00 | 34.24 | S |
| ATOM | 4861 | C | CYS | I | 196 | −25.209 | 23.781 | −4.243 | 1.00 | 32.63 | C |
| ATOM | 4862 | O | CYS | I | 196 | −26.187 | 24.413 | −4.651 | 1.00 | 32.58 | O |
| ATOM | 4863 | N | ASN | I | 197 | −24.144 | 24.343 | −3.667 | 1.00 | 32.20 | N |
| ATOM | 4864 | CA | ASN | I | 197 | −23.958 | 25.789 | −3.534 | 1.00 | 31.75 | C |
| ATOM | 4865 | CB | ASN | I | 197 | −23.452 | 26.144 | −2.138 | 1.00 | 31.50 | C |
| ATOM | 4866 | CG | ASN | I | 197 | −24.123 | 25.335 | −1.068 | 1.00 | 31.65 | C |
| ATOM | 4867 | OD1 | ASN | I | 197 | −23.576 | 24.338 | −0.614 | 1.00 | 31.54 | O |
| ATOM | 4868 | ND2 | ASN | I | 197 | −25.336 | 25.731 | −0.686 | 1.00 | 31.42 | N |
| ATOM | 4869 | C | ASN | I | 197 | −22.969 | 26.300 | −4.560 | 1.00 | 31.71 | C |
| ATOM | 4870 | O | ASN | I | 197 | −21.777 | 25.980 | −4.514 | 1.00 | 31.86 | O |
| ATOM | 4871 | N | VAL | I | 198 | −23.475 | 27.103 | −5.483 | 1.00 | 31.60 | N |
| ATOM | 4872 | CA | VAL | I | 198 | −22.678 | 27.593 | −6.582 | 1.00 | 31.60 | C |
| ATOM | 4873 | CB | VAL | I | 198 | −23.330 | 27.280 | −7.940 | 1.00 | 31.45 | C |
| ATOM | 4874 | CG1 | VAL | I | 198 | −22.448 | 27.763 | −9.074 | 1.00 | 31.31 | C |
| ATOM | 4875 | CG2 | VAL | I | 198 | −23.592 | 25.785 | −8.070 | 1.00 | 31.21 | C |
| ATOM | 4876 | C | VAL | I | 198 | −22.491 | 29.086 | −6.428 | 1.00 | 31.98 | C |
| ATOM | 4877 | O | VAL | I | 198 | −23.423 | 29.822 | −6.097 | 1.00 | 31.84 | O |
| ATOM | 4878 | N | ASN | I | 199 | −21.268 | 29.519 | −6.690 | 1.00 | 32.41 | N |
| ATOM | 4879 | CA | ASN | I | 199 | −20.864 | 30.885 | −6.469 | 1.00 | 33.01 | C |
| ATOM | 4880 | CB | ASN | I | 199 | −19.995 | 30.955 | −5.202 | 1.00 | 32.99 | C |
| ATOM | 4881 | CG | ASN | I | 199 | −19.848 | 32.362 | −4.652 | 1.00 | 34.68 | C |
| ATOM | 4882 | OD1 | ASN | I | 199 | −19.984 | 33.352 | −5.372 | 1.00 | 36.65 | O |
| ATOM | 4883 | ND2 | ASN | I | 199 | −19.543 | 32.456 | −3.360 | 1.00 | 36.71 | N |
| ATOM | 4884 | C | ASN | I | 199 | −20.092 | 31.328 | −7.701 | 1.00 | 32.89 | C |
| ATOM | 4885 | O | ASN | I | 199 | −19.100 | 30.696 | −8.069 | 1.00 | 33.13 | O |
| ATOM | 4886 | N | HIS | I | 200 | −20.574 | 32.381 | −8.358 | 1.00 | 32.85 | N |
| ATOM | 4887 | CA | HIS | I | 200 | −19.868 | 32.971 | −9.492 | 1.00 | 33.12 | C |
| ATOM | 4888 | CB | HIS | I | 200 | −20.631 | 32.757 | −10.798 | 1.00 | 32.70 | C |
| ATOM | 4889 | CG | HIS | I | 200 | −19.891 | 33.230 | −12.011 | 1.00 | 31.88 | C |
| ATOM | 4890 | ND1 | HIS | I | 200 | −20.420 | 34.141 | −12.897 | 1.00 | 30.05 | N |
| ATOM | 4891 | CE1 | HIS | I | 200 | −19.544 | 34.378 | −13.857 | 1.00 | 30.17 | C |
| ATOM | 4892 | NE2 | HIS | I | 200 | −18.463 | 33.659 | −13.623 | 1.00 | 29.90 | N |
| ATOM | 4893 | CD2 | HIS | I | 200 | −18.653 | 32.934 | −12.474 | 1.00 | 31.05 | C |
| ATOM | 4894 | C | HIS | I | 200 | −19.584 | 34.454 | −9.250 | 1.00 | 33.81 | C |
| ATOM | 4895 | O | HIS | I | 200 | −20.330 | 35.327 | −9.687 | 1.00 | 33.74 | O |
| ATOM | 4896 | N | LYS | I | 201 | −18.477 | 34.711 | −8.559 | 1.00 | 34.78 | N |
| ATOM | 4897 | CA | LYS | I | 201 | −18.091 | 36.048 | −8.119 | 1.00 | 35.64 | C |
| ATOM | 4898 | CB | LYS | I | 201 | −16.735 | 36.018 | −7.395 | 1.00 | 35.76 | C |
| ATOM | 4899 | CG | LYS | I | 201 | −16.757 | 35.193 | −6.102 | 1.00 | 36.99 | C |
| ATOM | 4900 | CD | LYS | I | 201 | −15.374 | 35.057 | −5.438 | 1.00 | 39.26 | C |
| ATOM | 4901 | CE | LYS | I | 201 | −15.469 | 34.311 | −4.086 | 1.00 | 40.12 | C |
| ATOM | 4902 | NZ | LYS | I | 201 | −14.142 | 33.911 | −3.495 | 1.00 | 40.10 | N |
| ATOM | 4903 | C | LYS | I | 201 | −18.102 | 37.092 | −9.228 | 1.00 | 36.05 | C |
| ATOM | 4904 | O | LYS | I | 201 | −18.603 | 38.195 | −9.010 | 1.00 | 36.33 | O |
| ATOM | 4905 | N | PRO | I | 202 | −17.573 | 36.760 | −10.426 | 1.00 | 36.68 | N |
| ATOM | 4906 | CA | PRO | I | 202 | −17.583 | 37.779 | −11.486 | 1.00 | 37.06 | C |
| ATOM | 4907 | CB | PRO | I | 202 | −17.016 | 37.025 | −12.691 | 1.00 | 36.84 | C |
| ATOM | 4908 | CG | PRO | I | 202 | −16.087 | 36.042 | −12.079 | 1.00 | 36.39 | C |
| ATOM | 4909 | CD | PRO | I | 202 | −16.809 | 35.567 | −10.850 | 1.00 | 36.53 | C |
| ATOM | 4910 | C | PRO | I | 202 | −18.952 | 38.394 | −11.809 | 1.00 | 37.45 | C |
| ATOM | 4911 | O | PRO | I | 202 | −19.015 | 39.569 | −12.135 | 1.00 | 37.85 | O |
| ATOM | 4912 | N | SER | I | 203 | −20.026 | 37.618 | −11.703 | 1.00 | 38.01 | N |
| ATOM | 4913 | CA | SER | I | 203 | −21.378 | 38.108 | −11.993 | 1.00 | 38.85 | C |
| ATOM | 4914 | CB | SER | I | 203 | −22.129 | 37.104 | −12.873 | 1.00 | 38.88 | C |
| ATOM | 4915 | OG | SER | I | 203 | −22.214 | 35.833 | −12.243 | 1.00 | 38.70 | O |
| ATOM | 4916 | C | SER | I | 203 | −22.195 | 38.381 | −10.730 | 1.00 | 39.64 | C |
| ATOM | 4917 | O | SER | I | 203 | −23.359 | 38.787 | −10.811 | 1.00 | 39.52 | O |
| ATOM | 4918 | N | ASN | I | 204 | −21.566 | 38.161 | −9.574 | 1.00 | 40.81 | N |
| ATOM | 4919 | CA | ASN | I | 204 | −22.215 | 38.228 | −8.254 | 1.00 | 42.10 | C |
| ATOM | 4920 | CB | ASN | I | 204 | −22.590 | 39.671 | −7.872 | 1.00 | 42.38 | C |
| ATOM | 4921 | CG | ASN | I | 204 | −21.368 | 40.559 | −7.666 | 1.00 | 43.63 | C |
| ATOM | 4922 | OD1 | ASN | I | 204 | −20.584 | 40.353 | −6.734 | 1.00 | 44.44 | O |
| ATOM | 4923 | ND2 | ASN | I | 204 | −21.203 | 41.556 | −8.539 | 1.00 | 44.41 | N |
| ATOM | 4924 | C | ASN | I | 204 | −23.406 | 37.285 | −8.116 | 1.00 | 42.55 | C |
| ATOM | 4925 | O | ASN | I | 204 | −24.418 | 37.626 | −7.489 | 1.00 | 42.43 | O |
| ATOM | 4926 | N | THR | I | 205 | −23.252 | 36.091 | −8.694 | 1.00 | 43.18 | N |
| ATOM | 4927 | CA | THR | I | 205 | −24.279 | 35.056 | −8.680 | 1.00 | 43.98 | C |
| ATOM | 4928 | CB | THR | I | 205 | −24.358 | 34.333 | −10.044 | 1.00 | 44.23 | C |
| ATOM | 4929 | OG1 | THR | I | 205 | −24.488 | 35.301 | −11.095 | 1.00 | 44.13 | O |
| ATOM | 4930 | CG2 | THR | I | 205 | −25.555 | 33.372 | −10.094 | 1.00 | 44.25 | C |
| ATOM | 4931 | C | THR | I | 205 | −24.047 | 34.034 | −7.564 | 1.00 | 44.46 | C |
| ATOM | 4932 | O | THR | I | 205 | −22.962 | 33.460 | −7.442 | 1.00 | 44.61 | O |
| ATOM | 4933 | N | LYS | I | 206 | −25.073 | 33.840 | −6.740 | 1.00 | 45.08 | N |
| ATOM | 4934 | CA | LYS | I | 206 | −25.096 | 32.783 | −5.732 | 1.00 | 45.63 | C |
| ATOM | 4935 | CB | LYS | I | 206 | −25.017 | 33.350 | −4.317 | 1.00 | 45.68 | C |
| ATOM | 4936 | CG | LYS | I | 206 | −23.616 | 33.599 | −3.818 | 1.00 | 46.63 | C |
| ATOM | 4937 | CD | LYS | I | 206 | −23.591 | 33.579 | −2.294 | 1.00 | 48.25 | C |
| ATOM | 4938 | CE | LYS | I | 206 | −22.192 | 33.852 | −1.753 | 1.00 | 49.46 | C |
| ATOM | 4939 | NZ | LYS | I | 206 | −22.015 | 33.291 | −0.381 | 1.00 | 49.73 | N |
| ATOM | 4940 | C | LYS | I | 206 | −26.379 | 31.989 | −5.894 | 1.00 | 45.83 | C |
| ATOM | 4941 | O | LYS | I | 206 | −27.473 | 32.539 | −5.791 | 1.00 | 46.16 | O |
| ATOM | 4942 | N | VAL | I | 207 | −26.240 | 30.698 | −6.159 | 1.00 | 45.93 | N |
| ATOM | 4943 | CA | VAL | I | 207 | −27.385 | 29.842 | −6.391 | 1.00 | 45.99 | C |
| ATOM | 4944 | CB | VAL | I | 207 | −27.462 | 29.414 | −7.876 | 1.00 | 46.07 | C |
| ATOM | 4945 | CG1 | VAL | I | 207 | −28.533 | 28.340 | −8.086 | 1.00 | 45.90 | C |
| ATOM | 4946 | CG2 | VAL | I | 207 | −27.732 | 30.612 | −8.763 | 1.00 | 45.88 | C |
| ATOM | 4947 | C | VAL | I | 207 | −27.282 | 28.613 | −5.505 | 1.00 | 46.31 | C |
| ATOM | 4948 | O | VAL | I | 207 | −26.260 | 27.920 | −5.507 | 1.00 | 46.39 | O |
| ATOM | 4949 | N | ASP | I | 208 | −28.334 | 28.350 | −4.738 | 1.00 | 46.47 | N |
| ATOM | 4950 | CA | ASP | I | 208 | −28.424 | 27.103 | −4.001 | 1.00 | 46.99 | C |
| ATOM | 4951 | CB | ASP | I | 208 | −28.721 | 27.356 | −2.523 | 1.00 | 47.01 | C |
| ATOM | 4952 | CG | ASP | I | 208 | −27.590 | 28.072 | −1.816 | 1.00 | 47.32 | C |
| ATOM | 4953 | OD1 | ASP | I | 208 | −26.420 | 27.890 | −2.207 | 1.00 | 47.73 | O |
| ATOM | 4954 | OD2 | ASP | I | 208 | −27.870 | 28.818 | −0.860 | 1.00 | 47.79 | O |
| ATOM | 4955 | C | ASP | I | 208 | −29.501 | 26.237 | −4.632 | 1.00 | 47.28 | C |
| ATOM | 4956 | O | ASP | I | 208 | −30.683 | 26.592 | −4.597 | 1.00 | 47.53 | O |
| ATOM | 4957 | N | LYS | I | 209 | −29.093 | 25.115 | −5.223 | 1.00 | 47.41 | N |
| ATOM | 4958 | CA | LYS | I | 209 | −30.038 | 24.230 | −5.886 | 1.00 | 48.02 | C |
| ATOM | 4959 | CB | LYS | I | 209 | −29.679 | 24.041 | −7.365 | 1.00 | 48.02 | C |
| ATOM | 4960 | CG | LYS | I | 209 | −30.863 | 23.639 | −8.261 | 1.00 | 48.24 | C |
| ATOM | 4961 | CD | LYS | I | 209 | −31.873 | 24.785 | −8.415 | 1.00 | 49.00 | C |
| ATOM | 4962 | CE | LYS | I | 209 | −32.627 | 24.716 | −9.742 | 1.00 | 48.89 | C |
| ATOM | 4963 | NZ | LYS | I | 209 | −33.523 | 25.893 | −9.939 | 1.00 | 47.98 | N |
| ATOM | 4964 | C | LYS | I | 209 | −30.157 | 22.886 | −5.191 | 1.00 | 48.43 | C |
| ATOM | 4965 | O | LYS | I | 209 | −29.187 | 22.133 | −5.097 | 1.00 | 48.20 | O |
| ATOM | 4966 | N | ARG | I | 210 | −31.366 | 22.600 | −4.716 | 1.00 | 49.26 | N |
| ATOM | 4967 | CA | ARG | I | 210 | −31.683 | 21.335 | −4.071 | 1.00 | 50.21 | C |
| ATOM | 4968 | CB | ARG | I | 210 | −33.000 | 21.454 | −3.307 | 1.00 | 50.28 | C |
| ATOM | 4969 | CG | ARG | I | 210 | −33.158 | 20.472 | −2.151 | 1.00 | 51.09 | C |
| ATOM | 4970 | CD | ARG | I | 210 | −34.547 | 20.593 | −1.534 | 1.00 | 52.57 | C |
| ATOM | 4971 | NE | ARG | I | 210 | −34.522 | 20.503 | −0.074 | 1.00 | 53.61 | N |
| ATOM | 4972 | CZ | ARG | I | 210 | −34.453 | 21.552 | 0.750 | 1.00 | 53.84 | C |
| ATOM | 4973 | NH1 | ARG | I | 210 | −34.399 | 22.789 | 0.268 | 1.00 | 53.18 | N |
| ATOM | 4974 | NH2 | ARG | I | 210 | −34.438 | 21.362 | 2.067 | 1.00 | 53.96 | N |
| ATOM | 4975 | C | ARG | I | 210 | −31.777 | 20.224 | −5.111 | 1.00 | 50.81 | C |
| ATOM | 4976 | O | ARG | I | 210 | −32.450 | 20.370 | −6.130 | 1.00 | 50.74 | O |
| ATOM | 4977 | N | VAL | I | 211 | −31.089 | 19.117 | −4.853 | 1.00 | 51.73 | N |
| ATOM | 4978 | CA | VAL | I | 211 | −31.124 | 17.974 | −5.754 | 1.00 | 52.54 | C |
| ATOM | 4979 | CB | VAL | I | 211 | −29.712 | 17.505 | −6.170 | 1.00 | 52.44 | C |
| ATOM | 4980 | CG1 | VAL | I | 211 | −29.801 | 16.353 | −7.165 | 1.00 | 52.61 | C |
| ATOM | 4981 | CG2 | VAL | I | 211 | −28.917 | 18.648 | −6.777 | 1.00 | 52.21 | C |
| ATOM | 4982 | C | VAL | I | 211 | −31.892 | 16.837 | −5.099 | 1.00 | 53.36 | C |
| ATOM | 4983 | O | VAL | I | 211 | −31.574 | 16.416 | −3.983 | 1.00 | 53.31 | O |
| ATOM | 4984 | N | GLU | I | 212 | −32.917 | 16.367 | −5.807 | 1.00 | 54.57 | N |
| ATOM | 4985 | CA | GLU | I | 212 | −33.762 | 15.251 | −5.374 | 1.00 | 55.65 | C |
| ATOM | 4986 | CB | GLU | I | 212 | −34.970 | 15.754 | −4.572 | 1.00 | 55.64 | C |
| ATOM | 4987 | CG | GLU | I | 212 | −35.642 | 17.023 | −5.103 | 1.00 | 55.43 | C |
| ATOM | 4988 | CD | GLU | I | 212 | −36.683 | 17.585 | −4.136 | 1.00 | 54.92 | C |
| ATOM | 4989 | OE1 | GLU | I | 212 | −37.006 | 16.906 | −3.141 | 1.00 | 54.06 | O |
| ATOM | 4990 | OE2 | GLU | I | 212 | −37.180 | 18.707 | −4.370 | 1.00 | 55.24 | O |
| ATOM | 4991 | C | GLU | I | 212 | −34.206 | 14.410 | −6.577 | 1.00 | 56.51 | C |
| ATOM | 4992 | O | GLU | I | 212 | −34.126 | 14.876 | −7.718 | 1.00 | 56.52 | O |
| ATOM | 4993 | N | PRO | I | 213 | −34.658 | 13.161 | −6.332 | 1.00 | 57.40 | N |
| ATOM | 4994 | CA | PRO | I | 213 | −35.073 | 12.271 | −7.426 | 1.00 | 57.99 | C |
| ATOM | 4995 | CB | PRO | I | 213 | −35.027 | 10.874 | −6.782 | 1.00 | 58.06 | C |
| ATOM | 4996 | CG | PRO | I | 213 | −34.591 | 11.077 | −5.343 | 1.00 | 57.89 | C |
| ATOM | 4997 | CD | PRO | I | 213 | −34.820 | 12.510 | −5.021 | 1.00 | 57.38 | C |
| ATOM | 4998 | C | PRO | I | 213 | −36.488 | 12.580 | −7.934 | 1.00 | 58.57 | C |
| ATOM | 4999 | O | PRO | I | 213 | −37.108 | 13.544 | −7.470 | 1.00 | 58.89 | O |
| ATOM | 5000 | N | LYS | I | 214 | −36.980 | 11.777 | −8.884 | 1.00 | 59.20 | N |
| ATOM | 5001 | CA | LYS | I | 214 | −38.360 | 11.880 | −9.407 | 1.00 | 59.62 | C |
| ATOM | 5002 | CB | LYS | I | 214 | −39.395 | 11.549 | −8.315 | 1.00 | 59.72 | C |
| ATOM | 5003 | CG | LYS | I | 214 | −40.852 | 11.697 | −8.744 | 1.00 | 60.31 | C |
| ATOM | 5004 | CD | LYS | I | 214 | −41.690 | 12.314 | −7.624 | 1.00 | 61.26 | C |
| ATOM | 5005 | CE | LYS | I | 214 | −43.136 | 12.551 | −8.067 | 1.00 | 61.95 | C |
| ATOM | 5006 | NZ | LYS | I | 214 | −43.259 | 13.493 | −9.230 | 1.00 | 61.39 | N |
| ATOM | 5007 | C | LYS | I | 214 | −38.664 | 13.244 | −10.031 | 1.00 | 59.66 | C |
| ATOM | 5008 | O | LYS | I | 214 | −37.762 | 14.040 | −10.286 | 1.00 | 59.85 | O |
| ATOM | 5009 | N | ASP | M | 1 | 17.902 | 28.815 | −30.566 | 1.00 | 42.93 | N |
| ATOM | 5010 | CA | ASP | M | 1 | 16.901 | 28.252 | −29.609 | 1.00 | 42.63 | C |
| ATOM | 5011 | CB | ASP | M | 1 | 15.464 | 28.433 | −30.145 | 1.00 | 42.89 | C |
| ATOM | 5012 | CG | ASP | M | 1 | 15.079 | 29.924 | −30.375 | 1.00 | 44.50 | C |
| ATOM | 5013 | OD1 | ASP | M | 1 | 15.954 | 30.734 | −30.789 | 1.00 | 44.12 | O |
| ATOM | 5014 | OD2 | ASP | M | 1 | 13.885 | 30.283 | −30.151 | 1.00 | 44.79 | O |
| ATOM | 5015 | C | ASP | M | 1 | 17.221 | 26.773 | −29.345 | 1.00 | 42.04 | C |
| ATOM | 5016 | O | ASP | M | 1 | 17.043 | 25.918 | −30.222 | 1.00 | 42.24 | O |
| ATOM | 5017 | N | VAL | M | 2 | 17.718 | 26.491 | −28.143 | 1.00 | 41.06 | N |
| ATOM | 5018 | CA | VAL | M | 2 | 18.064 | 25.132 | −27.716 | 1.00 | 40.08 | C |
| ATOM | 5019 | CB | VAL | M | 2 | 19.100 | 25.157 | −26.564 | 1.00 | 40.05 | C |
| ATOM | 5020 | CG1 | VAL | M | 2 | 19.618 | 23.760 | −26.266 | 1.00 | 39.67 | C |
| ATOM | 5021 | CG2 | VAL | M | 2 | 20.253 | 26.082 | −26.911 | 1.00 | 39.64 | C |
| ATOM | 5022 | C | VAL | M | 2 | 16.815 | 24.368 | −27.273 | 1.00 | 39.69 | C |
| ATOM | 5023 | O | VAL | M | 2 | 16.342 | 24.530 | −26.149 | 1.00 | 39.86 | O |
| ATOM | 5024 | N | VAL | M | 3 | 16.281 | 23.538 | −28.164 | 1.00 | 39.15 | N |
| ATOM | 5025 | CA | VAL | M | 3 | 15.071 | 22.762 | −27.872 | 1.00 | 38.43 | C |
| ATOM | 5026 | CB | VAL | M | 3 | 14.346 | 22.330 | −29.179 | 1.00 | 38.56 | C |
| ATOM | 5027 | CG1 | VAL | M | 3 | 13.559 | 21.025 | −28.997 | 1.00 | 38.38 | C |
| ATOM | 5028 | CG2 | VAL | M | 3 | 13.433 | 23.456 | −29.673 | 1.00 | 38.72 | C |
| ATOM | 5029 | C | VAL | M | 3 | 15.357 | 21.576 | −26.939 | 1.00 | 37.85 | C |
| ATOM | 5030 | O | VAL | M | 3 | 16.401 | 20.932 | −27.040 | 1.00 | 37.45 | O |
| ATOM | 5031 | N | MET | M | 4 | 14.411 | 21.303 | −26.037 | 1.00 | 37.46 | N |
| ATOM | 5032 | CA | MET | M | 4 | 14.588 | 20.314 | −24.972 | 1.00 | 36.84 | C |
| ATOM | 5033 | CB | MET | M | 4 | 14.554 | 20.997 | −23.604 | 1.00 | 36.80 | C |
| ATOM | 5034 | CG | MET | M | 4 | 15.476 | 22.198 | −23.518 | 1.00 | 37.64 | C |
| ATOM | 5035 | SD | MET | M | 4 | 16.835 | 22.040 | −22.360 | 1.00 | 39.85 | S |
| ATOM | 5036 | CE | MET | M | 4 | 17.662 | 20.561 | −22.891 | 1.00 | 40.41 | C |
| ATOM | 5037 | C | MET | M | 4 | 13.524 | 19.238 | −25.047 | 1.00 | 36.13 | C |
| ATOM | 5038 | O | MET | M | 4 | 12.321 | 19.533 | −24.974 | 1.00 | 36.18 | O |
| ATOM | 5039 | N | THR | M | 5 | 13.986 | 17.996 | −25.182 | 1.00 | 35.20 | N |
| ATOM | 5040 | CA | THR | M | 5 | 13.118 | 16.839 | −25.343 | 1.00 | 34.94 | C |
| ATOM | 5041 | CB | THR | M | 5 | 13.581 | 15.926 | −26.534 | 1.00 | 35.20 | C |
| ATOM | 5042 | OG1 | THR | M | 5 | 13.961 | 16.728 | −27.664 | 1.00 | 35.46 | O |
| ATOM | 5043 | CG2 | THR | M | 5 | 12.481 | 14.975 | −26.951 | 1.00 | 34.94 | C |
| ATOM | 5044 | C | THR | M | 5 | 13.097 | 16.023 | −24.045 | 1.00 | 34.67 | C |
| ATOM | 5045 | O | THR | M | 5 | 14.135 | 15.517 | −23.597 | 1.00 | 34.33 | O |
| ATOM | 5046 | N | GLN | M | 6 | 11.907 | 15.904 | −23.462 | 1.00 | 34.18 | N |
| ATOM | 5047 | CA | GLN | M | 6 | 11.687 | 15.142 | −22.239 | 1.00 | 34.24 | C |
| ATOM | 5048 | CB | GLN | M | 6 | 10.876 | 15.959 | −21.242 | 1.00 | 34.25 | C |
| ATOM | 5049 | CG | GLN | M | 6 | 11.669 | 16.606 | −20.160 | 1.00 | 33.26 | C |
| ATOM | 5050 | CD | GLN | M | 6 | 10.782 | 17.275 | −19.143 | 1.00 | 32.13 | C |
| ATOM | 5051 | OE1 | GLN | M | 6 | 10.527 | 18.470 | −19.228 | 1.00 | 30.78 | O |
| ATOM | 5052 | NE2 | GLN | M | 6 | 10.289 | 16.500 | −18.179 | 1.00 | 32.16 | N |
| ATOM | 5053 | C | GLN | M | 6 | 10.917 | 13.867 | −22.498 | 1.00 | 34.46 | C |
| ATOM | 5054 | O | GLN | M | 6 | 9.884 | 13.889 | −23.172 | 1.00 | 34.48 | O |
| ATOM | 5055 | N | THR | M | 7 | 11.406 | 12.768 | −21.929 | 1.00 | 34.60 | N |
| ATOM | 5056 | CA | THR | M | 7 | 10.729 | 11.471 | −21.999 | 1.00 | 34.91 | C |
| ATOM | 5057 | CB | THR | M | 7 | 11.363 | 10.527 | −23.063 | 1.00 | 35.08 | C |
| ATOM | 5058 | OG1 | THR | M | 7 | 12.704 | 10.197 | −22.672 | 1.00 | 34.87 | O |
| ATOM | 5059 | CG2 | THR | M | 7 | 11.360 | 11.162 | −24.468 | 1.00 | 35.01 | C |
| ATOM | 5060 | C | THR | M | 7 | 10.835 | 10.782 | −20.637 | 1.00 | 34.75 | C |
| ATOM | 5061 | O | THR | M | 7 | 11.871 | 10.902 | −19.978 | 1.00 | 35.26 | O |
| ATOM | 5062 | N | PRO | M | 8 | 9.778 | 10.065 | −20.208 | 1.00 | 34.30 | N |
| ATOM | 5063 | CA | PRO | M | 8 | 8.474 | 9.952 | −20.873 | 1.00 | 34.09 | C |
| ATOM | 5064 | CB | PRO | M | 8 | 7.873 | 8.687 | −20.255 | 1.00 | 33.85 | C |
| ATOM | 5065 | CG | PRO | M | 8 | 8.456 | 8.645 | −18.877 | 1.00 | 34.36 | C |
| ATOM | 5066 | CD | PRO | M | 8 | 9.851 | 9.235 | −18.992 | 1.00 | 33.92 | C |
| ATOM | 5067 | C | PRO | M | 8 | 7.600 | 11.132 | −20.515 | 1.00 | 34.04 | C |
| ATOM | 5068 | O | PRO | M | 8 | 7.893 | 11.833 | −19.543 | 1.00 | 33.95 | O |
| ATOM | 5069 | N | LEU | M | 9 | 6.532 | 11.334 | −21.281 | 1.00 | 33.83 | N |
| ATOM | 5070 | CA | LEU | M | 9 | 5.542 | 12.354 | −20.961 | 1.00 | 33.75 | C |
| ATOM | 5071 | CB | LEU | M | 9 | 4.700 | 12.732 | −22.200 | 1.00 | 33.92 | C |
| ATOM | 5072 | CG | LEU | M | 9 | 5.391 | 13.176 | −23.506 | 1.00 | 34.73 | C |
| ATOM | 5073 | CD1 | LEU | M | 9 | 4.358 | 13.551 | −24.575 | 1.00 | 35.04 | C |
| ATOM | 5074 | CD2 | LEU | M | 9 | 6.402 | 14.326 | −23.321 | 1.00 | 35.18 | C |
| ATOM | 5075 | C | LEU | M | 9 | 4.649 | 11.951 | −19.774 | 1.00 | 33.30 | C |
| ATOM | 5076 | O | LEU | M | 9 | 4.180 | 12.810 | −19.041 | 1.00 | 33.42 | O |
| ATOM | 5077 | N | SER | M | 10 | 4.413 | 10.657 | −19.589 | 1.00 | 32.85 | N |
| ATOM | 5078 | CA | SER | M | 10 | 3.615 | 10.173 | −18.454 | 1.00 | 32.59 | C |
| ATOM | 5079 | CB | SER | M | 10 | 2.281 | 9.579 | −18.907 | 1.00 | 32.39 | C |
| ATOM | 5080 | OG | SER | M | 10 | 1.585 | 10.481 | −19.729 | 1.00 | 33.51 | O |
| ATOM | 5081 | C | SER | M | 10 | 4.377 | 9.099 | −17.710 | 1.00 | 32.25 | C |
| ATOM | 5082 | O | SER | M | 10 | 4.900 | 8.173 | −18.322 | 1.00 | 32.19 | O |
| ATOM | 5083 | N | LEU | M | 11 | 4.421 | 9.217 | −16.387 | 1.00 | 31.82 | N |
| ATOM | 5084 | CA | LEU | M | 11 | 5.114 | 8.236 | −15.573 | 1.00 | 31.48 | C |
| ATOM | 5085 | CB | LEU | M | 11 | 6.515 | 8.741 | −15.195 | 1.00 | 31.32 | C |
| ATOM | 5086 | CG | LEU | M | 11 | 7.488 | 7.729 | −14.574 | 1.00 | 31.71 | C |
| ATOM | 5087 | CD1 | LEU | M | 11 | 7.636 | 6.443 | −15.423 | 1.00 | 30.16 | C |
| ATOM | 5088 | CD2 | LEU | M | 11 | 8.835 | 8.401 | −14.357 | 1.00 | 31.33 | C |
| ATOM | 5089 | C | LEU | M | 11 | 4.304 | 7.851 | −14.334 | 1.00 | 31.12 | C |
| ATOM | 5090 | O | LEU | M | 11 | 4.390 | 8.523 | −13.309 | 1.00 | 31.07 | O |
| ATOM | 5091 | N | PRO | M | 12 | 3.499 | 6.771 | −14.433 | 1.00 | 30.92 | N |
| ATOM | 5092 | CA | PRO | M | 12 | 2.879 | 6.220 | −13.228 | 1.00 | 30.65 | C |
| ATOM | 5093 | CB | PRO | M | 12 | 1.767 | 5.306 | −13.783 | 1.00 | 30.59 | C |
| ATOM | 5094 | CG | PRO | M | 12 | 1.679 | 5.615 | −15.254 | 1.00 | 30.45 | C |
| ATOM | 5095 | CD | PRO | M | 12 | 3.057 | 6.045 | −15.638 | 1.00 | 30.77 | C |
| ATOM | 5096 | C | PRO | M | 12 | 3.918 | 5.407 | −12.446 | 1.00 | 30.31 | C |
| ATOM | 5097 | O | PRO | M | 12 | 4.694 | 4.649 | −13.045 | 1.00 | 30.47 | O |
| ATOM | 5098 | N | VAL | M | 13 | 3.971 | 5.594 | −11.134 | 1.00 | 29.70 | N |
| ATOM | 5099 | CA | VAL | M | 13 | 4.932 | 4.865 | −10.313 | 1.00 | 29.30 | C |
| ATOM | 5100 | CB | VAL | M | 13 | 6.153 | 5.726 | −9.879 | 1.00 | 29.46 | C |
| ATOM | 5101 | CG1 | VAL | M | 13 | 7.357 | 4.831 | −9.582 | 1.00 | 28.94 | C |
| ATOM | 5102 | CG2 | VAL | M | 13 | 6.535 | 6.777 | −10.941 | 1.00 | 29.47 | C |
| ATOM | 5103 | C | VAL | M | 13 | 4.239 | 4.353 | −9.070 | 1.00 | 29.19 | C |
| ATOM | 5104 | O | VAL | M | 13 | 3.465 | 5.071 | −8.448 | 1.00 | 29.28 | O |
| ATOM | 5105 | N | THR | M | 14 | 4.502 | 3.095 | −8.732 | 1.00 | 29.25 | N |
| ATOM | 5106 | CA | THR | M | 14 | 4.079 | 2.526 | −7.463 | 1.00 | 29.20 | C |
| ATOM | 5107 | CB | THR | M | 14 | 4.384 | 0.997 | −7.420 | 1.00 | 29.14 | C |
| ATOM | 5108 | OG1 | THR | M | 14 | 3.715 | 0.338 | −8.497 | 1.00 | 28.52 | O |
| ATOM | 5109 | CG2 | THR | M | 14 | 3.942 | 0.375 | −6.105 | 1.00 | 29.54 | C |
| ATOM | 5110 | C | THR | M | 14 | 4.880 | 3.247 | −6.379 | 1.00 | 29.17 | C |
| ATOM | 5111 | O | THR | M | 14 | 6.091 | 3.341 | −6.493 | 1.00 | 29.08 | O |
| ATOM | 5112 | N | PRO | M | 15 | 4.217 | 3.771 | −5.333 | 1.00 | 29.52 | N |
| ATOM | 5113 | CA | PRO | M | 15 | 4.999 | 4.303 | −4.207 | 1.00 | 29.76 | C |
| ATOM | 5114 | CB | PRO | M | 15 | 3.949 | 4.553 | −3.119 | 1.00 | 29.61 | C |
| ATOM | 5115 | CG | PRO | M | 15 | 2.616 | 4.095 | −3.696 | 1.00 | 29.44 | C |
| ATOM | 5116 | CD | PRO | M | 15 | 2.775 | 4.024 | −5.174 | 1.00 | 29.64 | C |
| ATOM | 5117 | C | PRO | M | 15 | 6.044 | 3.282 | −3.736 | 1.00 | 30.13 | C |
| ATOM | 5118 | O | PRO | M | 15 | 5.760 | 2.082 | −3.732 | 1.00 | 30.40 | O |
| ATOM | 5119 | N | GLY | M | 16 | 7.242 | 3.765 | −3.389 | 1.00 | 30.34 | N |
| ATOM | 5120 | CA | GLY | M | 16 | 8.399 | 2.919 | −3.064 | 1.00 | 30.03 | C |
| ATOM | 5121 | C | GLY | M | 16 | 9.315 | 2.549 | −4.231 | 1.00 | 30.10 | C |
| ATOM | 5122 | O | GLY | M | 16 | 10.460 | 2.144 | −4.033 | 1.00 | 30.07 | O |
| ATOM | 5123 | N | GLU | M | 17 | 8.825 | 2.659 | −5.455 | 1.00 | 30.06 | N |
| ATOM | 5124 | CA | GLU | M | 17 | 9.648 | 2.281 | −6.601 | 1.00 | 30.51 | C |
| ATOM | 5125 | CB | GLU | M | 17 | 8.787 | 1.784 | −7.753 | 1.00 | 30.79 | C |
| ATOM | 5126 | CG | GLU | M | 17 | 7.757 | 0.751 | −7.333 | 1.00 | 34.41 | C |
| ATOM | 5127 | CD | GLU | M | 17 | 8.282 | −0.669 | −7.343 | 1.00 | 38.92 | C |
| ATOM | 5128 | OE1 | GLU | M | 17 | 7.584 | −1.527 | −7.940 | 1.00 | 40.93 | O |
| ATOM | 5129 | OE2 | GLU | M | 17 | 9.372 | −0.924 | −6.765 | 1.00 | 39.22 | O |
| ATOM | 5130 | C | GLU | M | 17 | 10.485 | 3.469 | −7.045 | 1.00 | 29.70 | C |
| ATOM | 5131 | O | GLU | M | 17 | 10.182 | 4.595 | −6.665 | 1.00 | 29.68 | O |
| ATOM | 5132 | N | PRO | M | 18 | 11.559 | 3.217 | −7.821 | 1.00 | 29.17 | N |
| ATOM | 5133 | CA | PRO | M | 18 | 12.354 | 4.318 | −8.354 | 1.00 | 28.65 | C |
| ATOM | 5134 | CB | PRO | M | 18 | 13.701 | 3.655 | −8.655 | 1.00 | 28.70 | C |
| ATOM | 5135 | CG | PRO | M | 18 | 13.377 | 2.235 | −8.915 | 1.00 | 28.19 | C |
| ATOM | 5136 | CD | PRO | M | 18 | 12.217 | 1.909 | −8.037 | 1.00 | 28.83 | C |
| ATOM | 5137 | C | PRO | M | 18 | 11.763 | 4.900 | −9.637 | 1.00 | 28.36 | C |
| ATOM | 5138 | O | PRO | M | 18 | 11.024 | 4.225 | −10.334 | 1.00 | 28.70 | O |
| ATOM | 5139 | N | ALA | M | 19 | 12.104 | 6.143 | −9.947 | 1.00 | 28.00 | N |
| ATOM | 5140 | CA | ALA | M | 19 | 11.699 | 6.765 | −11.197 | 1.00 | 27.40 | C |
| ATOM | 5141 | CB | ALA | M | 19 | 10.710 | 7.878 | −10.944 | 1.00 | 26.99 | C |
| ATOM | 5142 | C | ALA | M | 19 | 12.934 | 7.313 | −11.879 | 1.00 | 27.48 | C |
| ATOM | 5143 | O | ALA | M | 19 | 13.886 | 7.720 | −11.212 | 1.00 | 27.63 | O |
| ATOM | 5144 | N | SER | M | 20 | 12.908 | 7.306 | −13.206 | 1.00 | 27.42 | N |
| ATOM | 5145 | CA | SER | M | 20 | 13.954 | 7.891 | −14.016 | 1.00 | 27.54 | C |
| ATOM | 5146 | CB | SER | M | 20 | 14.773 | 6.778 | −14.662 | 1.00 | 27.73 | C |
| ATOM | 5147 | OG | SER | M | 20 | 15.746 | 7.275 | −15.559 | 1.00 | 28.35 | O |
| ATOM | 5148 | C | SER | M | 20 | 13.309 | 8.801 | −15.070 | 1.00 | 27.91 | C |
| ATOM | 5149 | O | SER | M | 20 | 12.342 | 8.420 | −15.738 | 1.00 | 28.03 | O |
| ATOM | 5150 | N | ILE | M | 21 | 13.830 | 10.017 | −15.197 | 1.00 | 27.83 | N |
| ATOM | 5151 | CA | ILE | M | 21 | 13.267 | 11.014 | −16.104 | 1.00 | 27.73 | C |
| ATOM | 5152 | CB | ILE | M | 21 | 12.488 | 12.111 | −15.335 | 1.00 | 27.64 | C |
| ATOM | 5153 | CG1 | ILE | M | 21 | 11.244 | 11.509 | −14.675 | 1.00 | 27.75 | C |
| ATOM | 5154 | CD1 | ILE | M | 21 | 10.550 | 12.427 | −13.710 | 1.00 | 27.09 | C |
| ATOM | 5155 | CG2 | ILE | M | 21 | 12.064 | 13.237 | −16.271 | 1.00 | 28.38 | C |
| ATOM | 5156 | C | ILE | M | 21 | 14.370 | 11.618 | −16.978 | 1.00 | 27.93 | C |
| ATOM | 5157 | O | ILE | M | 21 | 15.441 | 12.000 | −16.490 | 1.00 | 27.75 | O |
| ATOM | 5158 | N | SER | M | 22 | 14.097 | 11.722 | −18.273 | 1.00 | 27.98 | N |
| ATOM | 5159 | CA | SER | M | 22 | 15.156 | 11.977 | −19.224 | 1.00 | 28.23 | C |
| ATOM | 5160 | CB | SER | M | 22 | 15.308 | 10.755 | −20.116 | 1.00 | 28.10 | C |
| ATOM | 5161 | OG | SER | M | 22 | 16.543 | 10.812 | −20.765 | 1.00 | 29.42 | O |
| ATOM | 5162 | C | SER | M | 22 | 14.970 | 13.262 | −20.047 | 1.00 | 28.34 | C |
| ATOM | 5163 | O | SER | M | 22 | 13.845 | 13.647 | −20.396 | 1.00 | 28.12 | O |
| ATOM | 5164 | N | CYS | M | 23 | 16.086 | 13.924 | −20.340 | 1.00 | 28.40 | N |
| ATOM | 5165 | CA | CYS | M | 23 | 16.052 | 15.182 | −21.072 | 1.00 | 29.14 | C |
| ATOM | 5166 | CB | CYS | M | 23 | 15.974 | 16.369 | −20.085 | 1.00 | 28.99 | C |
| ATOM | 5167 | SG | CYS | M | 23 | 15.900 | 18.030 | −20.817 | 1.00 | 29.79 | S |
| ATOM | 5168 | C | CYS | M | 23 | 17.227 | 15.302 | −22.066 | 1.00 | 29.39 | C |
| ATOM | 5169 | O | CYS | M | 23 | 18.386 | 15.044 | −21.725 | 1.00 | 29.19 | O |
| ATOM | 5170 | N | THR | M | 24 | 16.903 | 15.682 | −23.300 | 1.00 | 30.14 | N |
| ATOM | 5171 | CA | THR | M | 24 | 17.883 | 15.799 | −24.383 | 1.00 | 31.13 | C |
| ATOM | 5172 | CB | THR | M | 24 | 17.669 | 14.662 | −25.449 | 1.00 | 31.26 | C |
| ATOM | 5173 | OG1 | THR | M | 24 | 18.522 | 13.556 | −25.132 | 1.00 | 30.83 | O |
| ATOM | 5174 | CG2 | THR | M | 24 | 17.987 | 15.126 | −26.871 | 1.00 | 31.72 | C |
| ATOM | 5175 | C | THR | M | 24 | 17.826 | 17.194 | −25.007 | 1.00 | 31.66 | C |
| ATOM | 5176 | O | THR | M | 24 | 16.735 | 17.716 | −25.251 | 1.00 | 32.00 | O |
| ATOM | 5177 | N | SER | M | 25 | 18.991 | 17.791 | −25.258 | 1.00 | 32.35 | N |
| ATOM | 5178 | CA | SER | M | 25 | 19.065 | 19.151 | −25.797 | 1.00 | 33.29 | C |
| ATOM | 5179 | CB | SER | M | 25 | 19.842 | 20.066 | −24.860 | 1.00 | 33.04 | C |
| ATOM | 5180 | OG | SER | M | 25 | 21.231 | 19.839 | −24.981 | 1.00 | 33.12 | O |
| ATOM | 5181 | C | SER | M | 25 | 19.706 | 19.217 | −27.173 | 1.00 | 34.31 | C |
| ATOM | 5182 | O | SER | M | 25 | 20.700 | 18.526 | −27.437 | 1.00 | 34.48 | O |
| ATOM | 5183 | N | GLY | M | 26 | 19.140 | 20.083 | −28.020 | 1.00 | 35.10 | N |
| ATOM | 5184 | CA | GLY | M | 26 | 19.613 | 20.326 | −29.380 | 1.00 | 36.35 | C |
| ATOM | 5185 | C | GLY | M | 26 | 21.108 | 20.562 | −29.476 | 1.00 | 37.27 | C |
| ATOM | 5186 | O | GLY | M | 26 | 21.794 | 19.948 | −30.310 | 1.00 | 37.90 | O |
| ATOM | 5187 | N | GLN | M | 27 | 21.622 | 21.446 | −28.623 | 1.00 | 37.65 | N |
| ATOM | 5188 | CA | GLN | M | 27 | 23.066 | 21.678 | −28.552 | 1.00 | 37.75 | C |
| ATOM | 5189 | CB | GLN | M | 27 | 23.474 | 22.961 | −29.300 | 1.00 | 38.17 | C |
| ATOM | 5190 | CG | GLN | M | 27 | 22.842 | 24.247 | −28.790 | 1.00 | 40.06 | C |
| ATOM | 5191 | CD | GLN | M | 27 | 22.136 | 25.034 | −29.901 | 1.00 | 43.36 | C |
| ATOM | 5192 | OE1 | GLN | M | 27 | 21.220 | 24.522 | −30.561 | 1.00 | 44.54 | O |
| ATOM | 5193 | NE2 | GLN | M | 27 | 22.550 | 26.287 | −30.098 | 1.00 | 43.11 | N |
| ATOM | 5194 | C | GLN | M | 27 | 23.597 | 21.661 | −27.121 | 1.00 | 37.20 | C |
| ATOM | 5195 | O | GLN | M | 27 | 22.841 | 21.819 | −26.156 | 1.00 | 36.78 | O |
| ATOM | 5196 | N | SER | M | 27A | 24.909 | 21.448 | −27.019 | 1.00 | 36.64 | N |
| ATOM | 5197 | CA | SER | M | 27A | 25.634 | 21.382 | −25.760 | 1.00 | 35.92 | C |
| ATOM | 5198 | CB | SER | M | 27A | 27.136 | 21.512 | −26.021 | 1.00 | 35.87 | C |
| ATOM | 5199 | OG | SER | M | 27A | 27.881 | 21.283 | −24.839 | 1.00 | 36.09 | O |
| ATOM | 5200 | C | SER | M | 27A | 25.190 | 22.475 | −24.798 | 1.00 | 35.32 | C |
| ATOM | 5201 | O | SER | M | 27A | 25.056 | 23.638 | −25.176 | 1.00 | 35.33 | O |
| ATOM | 5202 | N | LEU | M | 27B | 24.957 | 22.087 | −23.554 | 1.00 | 34.30 | N |
| ATOM | 5203 | CA | LEU | M | 27B | 24.634 | 23.052 | −22.526 | 1.00 | 33.48 | C |
| ATOM | 5204 | CB | LEU | M | 27B | 23.498 | 22.534 | −21.642 | 1.00 | 33.15 | C |
| ATOM | 5205 | CG | LEU | M | 27B | 22.201 | 22.128 | −22.341 | 1.00 | 32.61 | C |
| ATOM | 5206 | CD1 | LEU | M | 27B | 21.237 | 21.578 | −21.328 | 1.00 | 32.54 | C |
| ATOM | 5207 | CD2 | LEU | M | 27B | 21.554 | 23.279 | −23.089 | 1.00 | 33.15 | C |
| ATOM | 5208 | C | LEU | M | 27B | 25.885 | 23.417 | −21.713 | 1.00 | 33.02 | C |
| ATOM | 5209 | O | LEU | M | 27B | 25.799 | 24.074 | −20.667 | 1.00 | 32.64 | O |
| ATOM | 5210 | N | VAL | M | 27C | 27.045 | 22.997 | −22.210 | 1.00 | 32.47 | N |
| ATOM | 5211 | CA | VAL | M | 27C | 28.310 | 23.402 | −21.611 | 1.00 | 32.32 | C |
| ATOM | 5212 | CB | VAL | M | 27C | 29.506 | 22.477 | −22.000 | 1.00 | 32.38 | C |
| ATOM | 5213 | CG1 | VAL | M | 27C | 30.751 | 22.837 | −21.175 | 1.00 | 32.25 | C |
| ATOM | 5214 | CG2 | VAL | M | 27C | 29.161 | 21.009 | −21.794 | 1.00 | 32.00 | C |
| ATOM | 5215 | C | VAL | M | 27C | 28.604 | 24.848 | −22.004 | 1.00 | 32.05 | C |
| ATOM | 5216 | O | VAL | M | 27C | 28.859 | 25.153 | −23.170 | 1.00 | 31.71 | O |
| ATOM | 5217 | N | HIS | M | 27D | 28.532 | 25.740 | −21.022 | 1.00 | 31.95 | N |
| ATOM | 5218 | CA | HIS | M | 27D | 28.837 | 27.146 | −21.245 | 1.00 | 31.68 | C |
| ATOM | 5219 | CB | HIS | M | 27D | 28.523 | 27.956 | −19.991 | 1.00 | 31.16 | C |
| ATOM | 5220 | CG | HIS | M | 27D | 28.346 | 29.422 | −20.244 | 1.00 | 30.50 | C |
| ATOM | 5221 | ND1 | HIS | M | 27D | 29.339 | 30.347 | −20.002 | 1.00 | 29.67 | N |
| ATOM | 5222 | CE1 | HIS | M | 27D | 28.897 | 31.553 | −20.310 | 1.00 | 29.39 | C |
| ATOM | 5223 | NE2 | HIS | M | 27D | 27.655 | 31.444 | −20.747 | 1.00 | 28.71 | N |
| ATOM | 5224 | CD2 | HIS | M | 27D | 27.287 | 30.122 | −20.718 | 1.00 | 29.39 | C |
| ATOM | 5225 | C | HIS | M | 27D | 30.310 | 27.313 | −21.600 | 1.00 | 31.97 | C |
| ATOM | 5226 | O | HIS | M | 27D | 31.158 | 26.491 | −21.232 | 1.00 | 31.71 | O |
| ATOM | 5227 | N | ILE | M | 27E | 30.616 | 28.399 | −22.294 | 1.00 | 32.36 | N |
| ATOM | 5228 | CA | ILE | M | 27E | 31.998 | 28.722 | −22.582 | 1.00 | 32.94 | C |
| ATOM | 5229 | CB | ILE | M | 27E | 32.107 | 30.051 | −23.403 | 1.00 | 33.26 | C |
| ATOM | 5230 | CG1 | ILE | M | 27E | 32.921 | 29.825 | −24.686 | 1.00 | 34.22 | C |
| ATOM | 5231 | CD1 | ILE | M | 27E | 32.116 | 29.128 | −25.834 | 1.00 | 36.45 | C |
| ATOM | 5232 | CG2 | ILE | M | 27E | 32.599 | 31.245 | −22.545 | 1.00 | 34.25 | C |
| ATOM | 5233 | C | ILE | M | 27E | 32.851 | 28.724 | −21.292 | 1.00 | 32.60 | C |
| ATOM | 5234 | O | ILE | M | 27E | 34.022 | 28.316 | −21.316 | 1.00 | 33.08 | O |
| ATOM | 5235 | N | ASN | M | 28 | 32.256 | 29.148 | −20.173 | 1.00 | 31.55 | N |
| ATOM | 5236 | CA | ASN | M | 28 | 32.950 | 29.135 | −18.882 | 1.00 | 30.49 | C |
| ATOM | 5237 | CB | ASN | M | 28 | 32.271 | 30.079 | −17.878 | 1.00 | 30.63 | C |
| ATOM | 5238 | CG | ASN | M | 28 | 30.913 | 29.563 | −17.380 | 1.00 | 30.45 | C |
| ATOM | 5239 | OD1 | ASN | M | 28 | 30.725 | 28.371 | −17.120 | 1.00 | 29.45 | O |
| ATOM | 5240 | ND2 | ASN | M | 28 | 29.974 | 30.486 | −17.207 | 1.00 | 29.93 | N |
| ATOM | 5241 | C | ASN | M | 28 | 33.164 | 27.750 | −18.262 | 1.00 | 29.83 | C |
| ATOM | 5242 | O | ASN | M | 28 | 33.842 | 27.620 | −17.240 | 1.00 | 29.82 | O |
| ATOM | 5243 | N | GLY | M | 29 | 32.579 | 26.718 | −18.863 | 1.00 | 28.93 | N |
| ATOM | 5244 | CA | GLY | M | 29 | 32.811 | 25.356 | −18.397 | 1.00 | 27.95 | C |
| ATOM | 5245 | C | GLY | M | 29 | 31.702 | 24.737 | −17.561 | 1.00 | 27.47 | C |
| ATOM | 5246 | O | GLY | M | 29 | 31.665 | 23.515 | −17.402 | 1.00 | 28.00 | O |
| ATOM | 5247 | N | ASN | M | 30 | 30.808 | 25.555 | −17.008 | 1.00 | 26.29 | N |
| ATOM | 5248 | CA | ASN | M | 30 | 29.670 | 25.031 | −16.262 | 1.00 | 25.12 | C |
| ATOM | 5249 | CB | ASN | M | 30 | 29.142 | 26.069 | −15.265 | 1.00 | 25.15 | C |
| ATOM | 5250 | CG | ASN | M | 30 | 30.016 | 26.207 | −14.021 | 1.00 | 24.41 | C |
| ATOM | 5251 | OD1 | ASN | M | 30 | 30.891 | 25.385 | −13.758 | 1.00 | 26.12 | O |
| ATOM | 5252 | ND2 | ASN | M | 30 | 29.767 | 27.255 | −13.243 | 1.00 | 23.24 | N |
| ATOM | 5253 | C | ASN | M | 30 | 28.552 | 24.583 | −17.208 | 1.00 | 24.52 | C |
| ATOM | 5254 | O | ASN | M | 30 | 28.476 | 25.046 | −18.338 | 1.00 | 24.64 | O |
| ATOM | 5255 | N | THR | M | 31 | 27.705 | 23.668 | −16.740 | 1.00 | 23.86 | N |
| ATOM | 5256 | CA | THR | M | 31 | 26.524 | 23.212 | −17.476 | 1.00 | 23.06 | C |
| ATOM | 5257 | CB | THR | M | 31 | 26.494 | 21.650 | −17.693 | 1.00 | 23.41 | C |
| ATOM | 5258 | OG1 | THR | M | 31 | 27.698 | 21.185 | −18.328 | 1.00 | 23.24 | O |
| ATOM | 5259 | CG2 | THR | M | 31 | 25.299 | 21.259 | −18.559 | 1.00 | 22.86 | C |
| ATOM | 5260 | C | THR | M | 31 | 25.256 | 23.651 | −16.730 | 1.00 | 22.43 | C |
| ATOM | 5261 | O | THR | M | 31 | 24.966 | 23.186 | −15.620 | 1.00 | 21.99 | O |
| ATOM | 5262 | N | TYR | M | 32 | 24.502 | 24.544 | −17.357 | 1.00 | 22.02 | N |
| ATOM | 5263 | CA | TYR | M | 32 | 23.369 | 25.195 | −16.718 | 1.00 | 21.31 | C |
| ATOM | 5264 | CB | TYR | M | 32 | 23.342 | 26.681 | −17.084 | 1.00 | 21.18 | C |
| ATOM | 5265 | CG | TYR | M | 32 | 24.567 | 27.436 | −16.598 | 1.00 | 21.44 | C |
| ATOM | 5266 | CD1 | TYR | M | 32 | 24.692 | 27.812 | −15.263 | 1.00 | 21.83 | C |
| ATOM | 5267 | CE1 | TYR | M | 32 | 25.808 | 28.502 | −14.806 | 1.00 | 23.23 | C |
| ATOM | 5268 | CZ | TYR | M | 32 | 26.830 | 28.832 | −15.691 | 1.00 | 24.17 | C |
| ATOM | 5269 | OH | TYR | M | 32 | 27.935 | 29.525 | −15.228 | 1.00 | 23.84 | O |
| ATOM | 5270 | CE2 | TYR | M | 32 | 26.728 | 28.484 | −17.035 | 1.00 | 22.43 | C |
| ATOM | 5271 | CD2 | TYR | M | 32 | 25.600 | 27.780 | −17.478 | 1.00 | 22.44 | C |
| ATOM | 5272 | C | TYR | M | 32 | 22.059 | 24.481 | −17.054 | 1.00 | 21.04 | C |
| ATOM | 5273 | O | TYR | M | 32 | 21.166 | 25.041 | −17.702 | 1.00 | 20.70 | O |
| ATOM | 5274 | N | LEU | M | 33 | 21.971 | 23.228 | −16.596 | 1.00 | 20.50 | N |
| ATOM | 5275 | CA | LEU | M | 33 | 20.795 | 22.387 | −16.788 | 1.00 | 19.92 | C |
| ATOM | 5276 | CB | LEU | M | 33 | 21.187 | 21.089 | −17.535 | 1.00 | 20.01 | C |
| ATOM | 5277 | CG | LEU | M | 33 | 20.131 | 20.047 | −17.961 | 1.00 | 20.17 | C |
| ATOM | 5278 | CD1 | LEU | M | 33 | 18.898 | 20.688 | −18.615 | 1.00 | 18.62 | C |
| ATOM | 5279 | CD2 | LEU | M | 33 | 19.706 | 19.128 | −16.787 | 1.00 | 19.73 | C |
| ATOM | 5280 | C | LEU | M | 33 | 20.169 | 22.104 | −15.418 | 1.00 | 19.50 | C |
| ATOM | 5281 | O | LEU | M | 33 | 20.869 | 21.679 | −14.489 | 1.00 | 19.58 | O |
| ATOM | 5282 | N | HIS | M | 34 | 18.865 | 22.359 | −15.289 | 1.00 | 18.49 | N |
| ATOM | 5283 | CA | HIS | M | 34 | 18.180 | 22.279 | −13.993 | 1.00 | 18.29 | C |
| ATOM | 5284 | CB | HIS | M | 34 | 17.888 | 23.682 | −13.441 | 1.00 | 18.13 | C |
| ATOM | 5285 | CG | HIS | M | 34 | 18.869 | 24.716 | −13.888 | 1.00 | 18.34 | C |
| ATOM | 5286 | ND1 | HIS | M | 34 | 20.193 | 24.699 | −13.503 | 1.00 | 16.81 | N |
| ATOM | 5287 | CE1 | HIS | M | 34 | 20.821 | 25.714 | −14.068 | 1.00 | 16.24 | C |
| ATOM | 5288 | NE2 | HIS | M | 34 | 19.952 | 26.390 | −14.798 | 1.00 | 16.77 | N |
| ATOM | 5289 | CD2 | HIS | M | 34 | 18.726 | 25.782 | −14.710 | 1.00 | 17.00 | C |
| ATOM | 5290 | C | HIS | M | 34 | 16.886 | 21.465 | −14.028 | 1.00 | 18.13 | C |
| ATOM | 5291 | O | HIS | M | 34 | 16.292 | 21.266 | −15.085 | 1.00 | 18.26 | O |
| ATOM | 5292 | N | TRP | M | 35 | 16.444 | 21.003 | −12.866 | 1.00 | 17.48 | N |
| ATOM | 5293 | CA | TRP | M | 35 | 15.232 | 20.206 | −12.813 | 1.00 | 17.51 | C |
| ATOM | 5294 | CB | TRP | M | 35 | 15.531 | 18.769 | −12.363 | 1.00 | 17.39 | C |
| ATOM | 5295 | CG | TRP | M | 35 | 16.206 | 17.913 | −13.412 | 1.00 | 16.65 | C |
| ATOM | 5296 | CD1 | TRP | M | 35 | 17.540 | 17.600 | −13.493 | 1.00 | 15.40 | C |
| ATOM | 5297 | NE1 | TRP | M | 35 | 17.770 | 16.778 | −14.566 | 1.00 | 15.78 | N |
| ATOM | 5298 | CE2 | TRP | M | 35 | 16.582 | 16.551 | −15.212 | 1.00 | 16.68 | C |
| ATOM | 5299 | CD2 | TRP | M | 35 | 15.572 | 17.247 | −14.504 | 1.00 | 16.52 | C |
| ATOM | 5300 | CE3 | TRP | M | 35 | 14.249 | 17.175 | −14.960 | 1.00 | 16.53 | C |
| ATOM | 5301 | CZ3 | TRP | M | 35 | 13.977 | 16.429 | −16.090 | 1.00 | 18.28 | C |
| ATOM | 5302 | CH2 | TRP | M | 35 | 15.005 | 15.741 | −16.776 | 1.00 | 17.75 | C |
| ATOM | 5303 | CZ2 | TRP | M | 35 | 16.309 | 15.791 | −16.351 | 1.00 | 17.09 | C |
| ATOM | 5304 | C | TRP | M | 35 | 14.240 | 20.847 | −11.876 | 1.00 | 17.58 | C |
| ATOM | 5305 | O | TRP | M | 35 | 14.608 | 21.289 | −10.788 | 1.00 | 17.22 | O |
| ATOM | 5306 | N | TYR | M | 36 | 12.987 | 20.898 | −12.324 | 1.00 | 17.65 | N |
| ATOM | 5307 | CA | TYR | M | 36 | 11.888 | 21.465 | −11.553 | 1.00 | 17.86 | C |
| ATOM | 5308 | CB | TYR | M | 36 | 11.303 | 22.713 | −12.248 | 1.00 | 17.48 | C |
| ATOM | 5309 | CG | TYR | M | 36 | 12.206 | 23.900 | −12.158 | 1.00 | 17.21 | C |
| ATOM | 5310 | CD1 | TYR | M | 36 | 12.017 | 24.877 | −11.172 | 1.00 | 16.54 | C |
| ATOM | 5311 | CE1 | TYR | M | 36 | 12.871 | 25.969 | −11.073 | 1.00 | 14.51 | C |
| ATOM | 5312 | CZ | TYR | M | 36 | 13.929 | 26.087 | −11.964 | 1.00 | 15.23 | C |
| ATOM | 5313 | OH | TYR | M | 36 | 14.798 | 27.153 | −11.882 | 1.00 | 15.58 | O |
| ATOM | 5314 | CE2 | TYR | M | 36 | 14.134 | 25.133 | −12.940 | 1.00 | 15.07 | C |
| ATOM | 5315 | CD2 | TYR | M | 36 | 13.281 | 24.045 | −13.031 | 1.00 | 15.87 | C |
| ATOM | 5316 | C | TYR | M | 36 | 10.789 | 20.431 | −11.349 | 1.00 | 18.22 | C |
| ATOM | 5317 | O | TYR | M | 36 | 10.653 | 19.501 | −12.148 | 1.00 | 18.01 | O |
| ATOM | 5318 | N | LEU | M | 37 | 10.028 | 20.619 | −10.268 | 1.00 | 18.37 | N |
| ATOM | 5319 | CA | LEU | M | 37 | 8.770 | 19.932 | −10.024 | 1.00 | 18.98 | C |
| ATOM | 5320 | CB | LEU | M | 37 | 8.875 | 18.995 | −8.793 | 1.00 | 18.49 | C |
| ATOM | 5321 | CG | LEU | M | 37 | 7.562 | 18.385 | −8.254 | 1.00 | 18.08 | C |
| ATOM | 5322 | CD1 | LEU | M | 37 | 6.833 | 17.539 | −9.322 | 1.00 | 16.80 | C |
| ATOM | 5323 | CD2 | LEU | M | 37 | 7.729 | 17.566 | −6.971 | 1.00 | 16.66 | C |
| ATOM | 5324 | C | LEU | M | 37 | 7.672 | 20.983 | −9.795 | 1.00 | 19.66 | C |
| ATOM | 5325 | O | LEU | M | 37 | 7.809 | 21.872 | −8.962 | 1.00 | 19.61 | O |
| ATOM | 5326 | N | GLN | M | 38 | 6.583 | 20.876 | −10.540 | 1.00 | 20.73 | N |
| ATOM | 5327 | CA | GLN | M | 38 | 5.421 | 21.708 | −10.291 | 1.00 | 21.88 | C |
| ATOM | 5328 | CB | GLN | M | 38 | 4.929 | 22.376 | −11.586 | 1.00 | 21.65 | C |
| ATOM | 5329 | CG | GLN | M | 38 | 3.770 | 23.355 | −11.415 | 1.00 | 20.20 | C |
| ATOM | 5330 | CD | GLN | M | 38 | 3.337 | 23.999 | −12.725 | 1.00 | 20.72 | C |
| ATOM | 5331 | OE1 | GLN | M | 38 | 3.477 | 23.401 | −13.792 | 1.00 | 21.37 | O |
| ATOM | 5332 | NE2 | GLN | M | 38 | 2.801 | 25.222 | −12.650 | 1.00 | 18.40 | N |
| ATOM | 5333 | C | GLN | M | 38 | 4.344 | 20.806 | −9.725 | 1.00 | 22.88 | C |
| ATOM | 5334 | O | GLN | M | 38 | 3.887 | 19.900 | −10.405 | 1.00 | 23.34 | O |
| ATOM | 5335 | N | LYS | M | 39 | 3.959 | 21.054 | −8.477 | 1.00 | 24.22 | N |
| ATOM | 5336 | CA | LYS | M | 39 | 2.884 | 20.310 | −7.824 | 1.00 | 25.60 | C |
| ATOM | 5337 | CB | LYS | M | 39 | 3.060 | 20.310 | −6.294 | 1.00 | 25.57 | C |
| ATOM | 5338 | CG | LYS | M | 39 | 4.357 | 19.653 | −5.843 | 1.00 | 26.73 | C |
| ATOM | 5339 | CD | LYS | M | 39 | 4.485 | 19.502 | −4.322 | 1.00 | 28.11 | C |
| ATOM | 5340 | CE | LYS | M | 39 | 5.925 | 19.065 | −3.978 | 1.00 | 28.73 | C |
| ATOM | 5341 | NZ | LYS | M | 39 | 6.088 | 18.422 | −2.637 | 1.00 | 29.14 | N |
| ATOM | 5342 | C | LYS | M | 39 | 1.572 | 20.955 | −8.236 | 1.00 | 26.20 | C |
| ATOM | 5343 | O | LYS | M | 39 | 1.557 | 22.141 | −8.534 | 1.00 | 26.32 | O |
| ATOM | 5344 | N | PRO | M | 40 | 0.472 | 20.181 | −8.260 | 1.00 | 27.15 | N |
| ATOM | 5345 | CA | PRO | M | 40 | −0.826 | 20.661 | −8.771 | 1.00 | 27.75 | C |
| ATOM | 5346 | CB | PRO | M | 40 | −1.806 | 19.561 | −8.336 | 1.00 | 27.53 | C |
| ATOM | 5347 | CG | PRO | M | 40 | −0.984 | 18.354 | −8.162 | 1.00 | 27.36 | C |
| ATOM | 5348 | CD | PRO | M | 40 | 0.373 | 18.813 | −7.711 | 1.00 | 27.62 | C |
| ATOM | 5349 | C | PRO | M | 40 | −1.258 | 22.005 | −8.169 | 1.00 | 28.03 | C |
| ATOM | 5350 | O | PRO | M | 40 | −1.178 | 22.193 | −6.951 | 1.00 | 28.38 | O |
| ATOM | 5351 | N | GLY | M | 41 | −1.697 | 22.924 | −9.027 | 1.00 | 28.25 | N |
| ATOM | 5352 | CA | GLY | M | 41 | −2.137 | 24.261 | −8.601 | 1.00 | 28.32 | C |
| ATOM | 5353 | C | GLY | M | 41 | −1.052 | 25.211 | −8.095 | 1.00 | 28.33 | C |
| ATOM | 5354 | O | GLY | M | 41 | −1.364 | 26.291 | −7.577 | 1.00 | 28.22 | O |
| ATOM | 5355 | N | GLN | M | 42 | 0.214 | 24.826 | −8.263 | 1.00 | 27.70 | N |
| ATOM | 5356 | CA | GLN | M | 42 | 1.328 | 25.573 | −7.689 | 1.00 | 27.29 | C |
| ATOM | 5357 | CB | GLN | M | 42 | 1.958 | 24.786 | −6.541 | 1.00 | 27.50 | C |
| ATOM | 5358 | CG | GLN | M | 42 | 1.198 | 24.993 | −5.248 | 1.00 | 30.83 | C |
| ATOM | 5359 | CD | GLN | M | 42 | 1.159 | 23.754 | −4.376 | 1.00 | 34.76 | C |
| ATOM | 5360 | OE1 | GLN | M | 42 | 2.201 | 23.197 | −4.000 | 1.00 | 36.34 | O |
| ATOM | 5361 | NE2 | GLN | M | 42 | −0.055 | 23.316 | −4.038 | 1.00 | 35.85 | N |
| ATOM | 5362 | C | GLN | M | 42 | 2.389 | 25.993 | −8.695 | 1.00 | 26.34 | C |
| ATOM | 5363 | O | GLN | M | 42 | 2.408 | 25.540 | −9.833 | 1.00 | 26.43 | O |
| ATOM | 5364 | N | SER | M | 43 | 3.259 | 26.891 | −8.264 | 1.00 | 25.37 | N |
| ATOM | 5365 | CA | SER | M | 43 | 4.343 | 27.360 | −9.092 | 1.00 | 24.28 | C |
| ATOM | 5366 | CB | SER | M | 43 | 4.934 | 28.649 | −8.504 | 1.00 | 24.19 | C |
| ATOM | 5367 | OG | SER | M | 43 | 5.983 | 28.378 | −7.574 | 1.00 | 25.33 | O |
| ATOM | 5368 | C | SER | M | 43 | 5.386 | 26.239 | −9.137 | 1.00 | 23.45 | C |
| ATOM | 5369 | O | SER | M | 43 | 5.453 | 25.431 | −8.213 | 1.00 | 23.29 | O |
| ATOM | 5370 | N | PRO | M | 44 | 6.182 | 26.169 | −10.216 | 1.00 | 22.61 | N |
| ATOM | 5371 | CA | PRO | M | 44 | 7.288 | 25.217 | −10.263 | 1.00 | 22.32 | C |
| ATOM | 5372 | CB | PRO | M | 44 | 7.971 | 25.561 | −11.584 | 1.00 | 22.33 | C |
| ATOM | 5373 | CG | PRO | M | 44 | 6.874 | 26.127 | −12.420 | 1.00 | 22.37 | C |
| ATOM | 5374 | CD | PRO | M | 44 | 6.024 | 26.899 | −11.485 | 1.00 | 21.86 | C |
| ATOM | 5375 | C | PRO | M | 44 | 8.265 | 25.428 | −9.109 | 1.00 | 22.45 | C |
| ATOM | 5376 | O | PRO | M | 44 | 8.461 | 26.574 | −8.683 | 1.00 | 23.12 | O |
| ATOM | 5377 | N | LYS | M | 45 | 8.850 | 24.341 | −8.601 | 1.00 | 22.14 | N |
| ATOM | 5378 | CA | LYS | M | 45 | 9.895 | 24.408 | −7.562 | 1.00 | 22.49 | C |
| ATOM | 5379 | CB | LYS | M | 45 | 9.431 | 23.754 | −6.253 | 1.00 | 22.92 | C |
| ATOM | 5380 | CG | LYS | M | 45 | 8.551 | 24.656 | −5.411 | 1.00 | 26.25 | C |
| ATOM | 5381 | CD | LYS | M | 45 | 7.748 | 23.878 | −4.369 | 1.00 | 31.20 | C |
| ATOM | 5382 | CE | LYS | M | 45 | 6.294 | 24.381 | −4.287 | 1.00 | 32.24 | C |
| ATOM | 5383 | NZ | LYS | M | 45 | 6.224 | 25.885 | −4.332 | 1.00 | 34.03 | N |
| ATOM | 5384 | C | LYS | M | 45 | 11.204 | 23.770 | −8.005 | 1.00 | 21.62 | C |
| ATOM | 5385 | O | LYS | M | 45 | 11.215 | 22.712 | −8.639 | 1.00 | 21.65 | O |
| ATOM | 5386 | N | LEU | M | 46 | 12.307 | 24.414 | −7.655 | 1.00 | 20.79 | N |
| ATOM | 5387 | CA | LEU | M | 46 | 13.627 | 23.971 | −8.095 | 1.00 | 19.99 | C |
| ATOM | 5388 | CB | LEU | M | 46 | 14.619 | 25.132 | −8.048 | 1.00 | 19.65 | C |
| ATOM | 5389 | CG | LEU | M | 46 | 16.086 | 24.824 | −8.341 | 1.00 | 19.54 | C |
| ATOM | 5390 | CD1 | LEU | M | 46 | 16.310 | 24.477 | −9.800 | 1.00 | 19.00 | C |
| ATOM | 5391 | CD2 | LEU | M | 46 | 16.941 | 26.007 | −7.940 | 1.00 | 18.98 | C |
| ATOM | 5392 | C | LEU | M | 46 | 14.129 | 22.796 | −7.267 | 1.00 | 19.57 | C |
| ATOM | 5393 | O | LEU | M | 46 | 14.144 | 22.841 | −6.030 | 1.00 | 19.82 | O |
| ATOM | 5394 | N | LEU | M | 47 | 14.537 | 21.740 | −7.958 | 1.00 | 19.14 | N |
| ATOM | 5395 | CA | LEU | M | 47 | 15.011 | 20.535 | −7.302 | 1.00 | 18.18 | C |
| ATOM | 5396 | CB | LEU | M | 47 | 14.377 | 19.302 | −7.936 | 1.00 | 17.84 | C |
| ATOM | 5397 | CG | LEU | M | 47 | 12.887 | 19.070 | −7.717 | 1.00 | 17.00 | C |
| ATOM | 5398 | CD1 | LEU | M | 47 | 12.519 | 17.705 | −8.258 | 1.00 | 16.91 | C |
| ATOM | 5399 | CD2 | LEU | M | 47 | 12.493 | 19.185 | −6.251 | 1.00 | 15.13 | C |
| ATOM | 5400 | C | LEU | M | 47 | 16.518 | 20.433 | −7.379 | 1.00 | 18.20 | C |
| ATOM | 5401 | O | LEU | M | 47 | 17.196 | 20.300 | −6.353 | 1.00 | 17.54 | O |
| ATOM | 5402 | N | ILE | M | 48 | 17.023 | 20.480 | −8.614 | 1.00 | 18.24 | N |
| ATOM | 5403 | CA | ILE | M | 48 | 18.435 | 20.311 | −8.914 | 1.00 | 18.22 | C |
| ATOM | 5404 | CB | ILE | M | 48 | 18.685 | 18.941 | −9.591 | 1.00 | 18.63 | C |
| ATOM | 5405 | CG1 | ILE | M | 48 | 18.163 | 17.798 | −8.720 | 1.00 | 20.53 | C |
| ATOM | 5406 | CD1 | ILE | M | 48 | 18.232 | 16.427 | −9.399 | 1.00 | 23.12 | C |
| ATOM | 5407 | CG2 | ILE | M | 48 | 20.176 | 18.716 | −9.873 | 1.00 | 19.11 | C |
| ATOM | 5408 | C | ILE | M | 48 | 18.875 | 21.425 | −9.861 | 1.00 | 17.79 | C |
| ATOM | 5409 | O | ILE | M | 48 | 18.161 | 21.744 | −10.808 | 1.00 | 17.58 | O |
| ATOM | 5410 | N | TYR | M | 49 | 20.050 | 22.005 | −9.604 | 1.00 | 17.67 | N |
| ATOM | 5411 | CA | TYR | M | 49 | 20.642 | 22.996 | −10.500 | 1.00 | 17.64 | C |
| ATOM | 5412 | CB | TYR | M | 49 | 20.701 | 24.397 | −9.859 | 1.00 | 17.40 | C |
| ATOM | 5413 | CG | TYR | M | 49 | 21.636 | 24.536 | −8.678 | 1.00 | 17.81 | C |
| ATOM | 5414 | CD1 | TYR | M | 49 | 22.989 | 24.828 | −8.861 | 1.00 | 19.05 | C |
| ATOM | 5415 | CE1 | TYR | M | 49 | 23.861 | 24.959 | −7.774 | 1.00 | 18.14 | C |
| ATOM | 5416 | CZ | TYR | M | 49 | 23.376 | 24.816 | −6.487 | 1.00 | 19.37 | C |
| ATOM | 5417 | OH | TYR | M | 49 | 24.231 | 24.943 | −5.410 | 1.00 | 19.41 | O |
| ATOM | 5418 | CE2 | TYR | M | 49 | 22.035 | 24.528 | −6.279 | 1.00 | 18.75 | C |
| ATOM | 5419 | CD2 | TYR | M | 49 | 21.173 | 24.394 | −7.378 | 1.00 | 18.30 | C |
| ATOM | 5420 | C | TYR | M | 49 | 22.014 | 22.544 | −10.953 | 1.00 | 17.78 | C |
| ATOM | 5421 | O | TYR | M | 49 | 22.721 | 21.844 | −10.220 | 1.00 | 18.29 | O |
| ATOM | 5422 | N | LYS | M | 50 | 22.390 | 22.959 | −12.162 | 1.00 | 17.73 | N |
| ATOM | 5423 | CA | LYS | M | 50 | 23.673 | 22.587 | −12.784 | 1.00 | 17.31 | C |
| ATOM | 5424 | CB | LYS | M | 50 | 24.851 | 23.370 | −12.195 | 1.00 | 16.95 | C |
| ATOM | 5425 | CG | LYS | M | 50 | 24.894 | 24.832 | −12.613 | 1.00 | 16.87 | C |
| ATOM | 5426 | CD | LYS | M | 50 | 26.259 | 25.466 | −12.398 | 1.00 | 15.37 | C |
| ATOM | 5427 | CE | LYS | M | 50 | 26.660 | 25.463 | −10.920 | 1.00 | 16.98 | C |
| ATOM | 5428 | NZ | LYS | M | 50 | 27.913 | 26.229 | −10.698 | 1.00 | 16.62 | N |
| ATOM | 5429 | C | LYS | M | 50 | 23.924 | 21.087 | −12.740 | 1.00 | 17.32 | C |
| ATOM | 5430 | O | LYS | M | 50 | 24.929 | 20.618 | −12.184 | 1.00 | 17.70 | O |
| ATOM | 5431 | N | VAL | M | 51 | 22.983 | 20.345 | −13.321 | 1.00 | 17.22 | N |
| ATOM | 5432 | CA | VAL | M | 51 | 23.097 | 18.889 | −13.532 | 1.00 | 17.20 | C |
| ATOM | 5433 | CB | VAL | M | 51 | 24.491 | 18.467 | −14.172 | 1.00 | 17.25 | C |
| ATOM | 5434 | CG1 | VAL | M | 51 | 24.554 | 16.963 | −14.459 | 1.00 | 16.35 | C |
| ATOM | 5435 | CG2 | VAL | M | 51 | 24.790 | 19.275 | −15.428 | 1.00 | 16.25 | C |
| ATOM | 5436 | C | VAL | M | 51 | 22.800 | 18.055 | −12.278 | 1.00 | 17.19 | C |
| ATOM | 5437 | O | VAL | M | 51 | 21.890 | 17.226 | −12.295 | 1.00 | 16.92 | O |
| ATOM | 5438 | N | SER | M | 52 | 23.541 | 18.305 | −11.199 | 1.00 | 17.39 | N |
| ATOM | 5439 | CA | SER | M | 52 | 23.593 | 17.383 | −10.062 | 1.00 | 17.79 | C |
| ATOM | 5440 | CB | SER | M | 52 | 24.855 | 16.526 | −10.185 | 1.00 | 17.91 | C |
| ATOM | 5441 | OG | SER | M | 52 | 26.000 | 17.323 | −9.926 | 1.00 | 16.72 | O |
| ATOM | 5442 | C | SER | M | 52 | 23.603 | 18.000 | −8.651 | 1.00 | 18.10 | C |
| ATOM | 5443 | O | SER | M | 52 | 23.745 | 17.267 | −7.682 | 1.00 | 18.69 | O |
| ATOM | 5444 | N | ASN | M | 53 | 23.508 | 19.319 | −8.516 | 1.00 | 18.23 | N |
| ATOM | 5445 | CA | ASN | M | 53 | 23.506 | 19.935 | −7.184 | 1.00 | 17.97 | C |
| ATOM | 5446 | CB | ASN | M | 53 | 24.177 | 21.312 | −7.205 | 1.00 | 18.34 | C |
| ATOM | 5447 | CG | ASN | M | 53 | 25.518 | 21.309 | −7.921 | 1.00 | 19.11 | C |
| ATOM | 5448 | OD1 | ASN | M | 53 | 25.594 | 21.697 | −9.076 | 1.00 | 21.89 | O |
| ATOM | 5449 | ND2 | ASN | M | 53 | 26.573 | 20.865 | −7.244 | 1.00 | 18.39 | N |
| ATOM | 5450 | C | ASN | M | 53 | 22.080 | 20.080 | −6.696 | 1.00 | 17.95 | C |
| ATOM | 5451 | O | ASN | M | 53 | 21.240 | 20.650 | −7.403 | 1.00 | 18.03 | O |
| ATOM | 5452 | N | LEU | M | 54 | 21.790 | 19.561 | −5.506 | 1.00 | 17.86 | N |
| ATOM | 5453 | CA | LEU | M | 54 | 20.450 | 19.696 | −4.924 | 1.00 | 17.83 | C |
| ATOM | 5454 | CB | LEU | M | 54 | 20.200 | 18.672 | −3.805 | 1.00 | 17.89 | C |
| ATOM | 5455 | CG | LEU | M | 54 | 20.458 | 17.164 | −4.011 | 1.00 | 19.54 | C |
| ATOM | 5456 | CD1 | LEU | M | 54 | 19.968 | 16.360 | −2.789 | 1.00 | 19.65 | C |
| ATOM | 5457 | CD2 | LEU | M | 54 | 19.816 | 16.621 | −5.275 | 1.00 | 19.44 | C |
| ATOM | 5458 | C | LEU | M | 54 | 20.253 | 21.100 | −4.383 | 1.00 | 17.71 | C |
| ATOM | 5459 | O | LEU | M | 54 | 21.180 | 21.703 | −3.868 | 1.00 | 17.82 | O |
| ATOM | 5460 | N | PHE | M | 55 | 19.045 | 21.619 | −4.524 | 1.00 | 18.06 | N |
| ATOM | 5461 | CA | PHE | M | 55 | 18.687 | 22.905 | −3.962 | 1.00 | 18.75 | C |
| ATOM | 5462 | CB | PHE | M | 55 | 17.502 | 23.497 | −4.738 | 1.00 | 18.18 | C |
| ATOM | 5463 | CG | PHE | M | 55 | 17.189 | 24.928 | −4.393 | 1.00 | 17.23 | C |
| ATOM | 5464 | CD1 | PHE | M | 55 | 18.102 | 25.945 | −4.660 | 1.00 | 14.39 | C |
| ATOM | 5465 | CE1 | PHE | M | 55 | 17.802 | 27.275 | −4.350 | 1.00 | 13.63 | C |
| ATOM | 5466 | CZ | PHE | M | 55 | 16.585 | 27.600 | −3.757 | 1.00 | 13.28 | C |
| ATOM | 5467 | CE2 | PHE | M | 55 | 15.661 | 26.603 | −3.493 | 1.00 | 15.86 | C |
| ATOM | 5468 | CD2 | PHE | M | 55 | 15.960 | 25.267 | −3.819 | 1.00 | 16.73 | C |
| ATOM | 5469 | C | PHE | M | 55 | 18.335 | 22.694 | −2.486 | 1.00 | 19.75 | C |
| ATOM | 5470 | O | PHE | M | 55 | 17.909 | 21.605 | −2.086 | 1.00 | 20.24 | O |
| ATOM | 5471 | N | SER | M | 56 | 18.523 | 23.739 | −1.685 | 1.00 | 20.54 | N |
| ATOM | 5472 | CA | SER | M | 56 | 18.287 | 23.711 | −0.241 | 1.00 | 21.32 | C |
| ATOM | 5473 | CB | SER | M | 56 | 18.322 | 25.136 | 0.296 | 1.00 | 21.54 | C |
| ATOM | 5474 | OG | SER | M | 56 | 19.095 | 25.203 | 1.480 | 1.00 | 24.88 | O |
| ATOM | 5475 | C | SER | M | 56 | 16.949 | 23.085 | 0.124 | 1.00 | 21.15 | C |
| ATOM | 5476 | O | SER | M | 56 | 15.917 | 23.511 | −0.375 | 1.00 | 21.45 | O |
| ATOM | 5477 | N | GLY | M | 57 | 16.973 | 22.073 | 0.989 | 1.00 | 21.19 | N |
| ATOM | 5478 | CA | GLY | M | 57 | 15.741 | 21.456 | 1.488 | 1.00 | 20.76 | C |
| ATOM | 5479 | C | GLY | M | 57 | 15.280 | 20.258 | 0.667 | 1.00 | 20.85 | C |
| ATOM | 5480 | O | GLY | M | 57 | 14.244 | 19.650 | 0.974 | 1.00 | 20.81 | O |
| ATOM | 5481 | N | VAL | M | 58 | 16.038 | 19.913 | −0.378 | 1.00 | 20.02 | N |
| ATOM | 5482 | CA | VAL | M | 58 | 15.612 | 18.852 | −1.279 | 1.00 | 19.62 | C |
| ATOM | 5483 | CB | VAL | M | 58 | 15.850 | 19.225 | −2.778 | 1.00 | 19.74 | C |
| ATOM | 5484 | CG1 | VAL | M | 58 | 15.596 | 18.056 | −3.702 | 1.00 | 18.90 | C |
| ATOM | 5485 | CG2 | VAL | M | 58 | 14.946 | 20.380 | −3.174 | 1.00 | 19.36 | C |
| ATOM | 5486 | C | VAL | M | 58 | 16.262 | 17.542 | −0.847 | 1.00 | 19.65 | C |
| ATOM | 5487 | O | VAL | M | 58 | 17.487 | 17.468 | −0.691 | 1.00 | 19.50 | O |
| ATOM | 5488 | N | PRO | M | 59 | 15.434 | 16.511 | −0.611 | 1.00 | 19.60 | N |
| ATOM | 5489 | CA | PRO | M | 59 | 15.934 | 15.258 | −0.049 | 1.00 | 19.62 | C |
| ATOM | 5490 | CB | PRO | M | 59 | 14.653 | 14.475 | 0.256 | 1.00 | 19.19 | C |
| ATOM | 5491 | CG | PRO | M | 59 | 13.660 | 14.997 | −0.707 | 1.00 | 19.26 | C |
| ATOM | 5492 | CD | PRO | M | 59 | 13.985 | 16.456 | −0.873 | 1.00 | 19.68 | C |
| ATOM | 5493 | C | PRO | M | 59 | 16.829 | 14.492 | −1.027 | 1.00 | 19.83 | C |
| ATOM | 5494 | O | PRO | M | 59 | 16.613 | 14.558 | −2.252 | 1.00 | 19.91 | O |
| ATOM | 5495 | N | ASP | M | 60 | 17.808 | 13.766 | −0.480 | 1.00 | 19.81 | N |
| ATOM | 5496 | CA | ASP | M | 60 | 18.781 | 13.007 | −1.283 | 1.00 | 19.62 | C |
| ATOM | 5497 | CB | ASP | M | 60 | 19.986 | 12.569 | −0.434 | 1.00 | 19.78 | C |
| ATOM | 5498 | CG | ASP | M | 60 | 19.620 | 11.641 | 0.729 | 1.00 | 21.13 | C |
| ATOM | 5499 | OD1 | ASP | M | 60 | 18.566 | 10.957 | 0.709 | 1.00 | 21.76 | O |
| ATOM | 5500 | OD2 | ASP | M | 60 | 20.433 | 11.584 | 1.683 | 1.00 | 23.46 | O |
| ATOM | 5501 | C | ASP | M | 60 | 18.218 | 11.850 | −2.118 | 1.00 | 19.30 | C |
| ATOM | 5502 | O | ASP | M | 60 | 18.967 | 11.166 | −2.811 | 1.00 | 19.16 | O |
| ATOM | 5503 | N | ARG | M | 61 | 16.906 | 11.643 | −2.089 | 1.00 | 19.15 | N |
| ATOM | 5504 | CA | ARG | M | 61 | 16.314 | 10.628 | −2.969 | 1.00 | 19.60 | C |
| ATOM | 5505 | CB | ARG | M | 61 | 14.928 | 10.155 | −2.481 | 1.00 | 19.67 | C |
| ATOM | 5506 | CG | ARG | M | 61 | 13.827 | 11.200 | −2.402 | 1.00 | 20.15 | C |
| ATOM | 5507 | CD | ARG | M | 61 | 12.445 | 10.513 | −2.334 | 1.00 | 23.30 | C |
| ATOM | 5508 | NE | ARG | M | 61 | 11.334 | 11.459 | −2.339 | 1.00 | 23.64 | N |
| ATOM | 5509 | CZ | ARG | M | 61 | 10.965 | 12.170 | −1.279 | 1.00 | 26.14 | C |
| ATOM | 5510 | NH1 | ARG | M | 61 | 11.612 | 12.044 | −0.118 | 1.00 | 28.54 | N |
| ATOM | 5511 | NH2 | ARG | M | 61 | 9.959 | 13.020 | −1.373 | 1.00 | 26.48 | N |
| ATOM | 5512 | C | ARG | M | 61 | 16.291 | 11.082 | −4.441 | 1.00 | 19.52 | C |
| ATOM | 5513 | O | ARG | M | 61 | 16.222 | 10.246 | −5.360 | 1.00 | 19.95 | O |
| ATOM | 5514 | N | PHE | M | 62 | 16.348 | 12.402 | −4.646 | 1.00 | 18.57 | N |
| ATOM | 5515 | CA | PHE | M | 62 | 16.532 | 12.982 | −5.967 | 1.00 | 17.91 | C |
| ATOM | 5516 | CB | PHE | M | 62 | 15.858 | 14.364 | −6.094 | 1.00 | 17.56 | C |
| ATOM | 5517 | CG | PHE | M | 62 | 14.360 | 14.344 | −5.856 | 1.00 | 17.08 | C |
| ATOM | 5518 | CD1 | PHE | M | 62 | 13.472 | 14.155 | −6.912 | 1.00 | 17.04 | C |
| ATOM | 5519 | CE1 | PHE | M | 62 | 12.087 | 14.123 | −6.696 | 1.00 | 17.59 | C |
| ATOM | 5520 | CZ | PHE | M | 62 | 11.573 | 14.278 | −5.400 | 1.00 | 17.19 | C |
| ATOM | 5521 | CE2 | PHE | M | 62 | 12.453 | 14.468 | −4.340 | 1.00 | 17.51 | C |
| ATOM | 5522 | CD2 | PHE | M | 62 | 13.841 | 14.499 | −4.574 | 1.00 | 17.07 | C |
| ATOM | 5523 | C | PHE | M | 62 | 18.023 | 13.079 | −6.240 | 1.00 | 17.84 | C |
| ATOM | 5524 | O | PHE | M | 62 | 18.828 | 13.326 | −5.335 | 1.00 | 17.63 | O |
| ATOM | 5525 | N | SER | M | 63 | 18.383 | 12.832 | −7.489 | 1.00 | 17.86 | N |
| ATOM | 5526 | CA | SER | M | 63 | 19.732 | 13.062 | −7.981 | 1.00 | 18.06 | C |
| ATOM | 5527 | CB | SER | M | 63 | 20.664 | 11.906 | −7.624 | 1.00 | 17.82 | C |
| ATOM | 5528 | OG | SER | M | 63 | 20.180 | 10.711 | −8.179 | 1.00 | 18.01 | O |
| ATOM | 5529 | C | SER | M | 63 | 19.675 | 13.227 | −9.482 | 1.00 | 18.31 | C |
| ATOM | 5530 | O | SER | M | 63 | 18.795 | 12.668 | −10.164 | 1.00 | 18.63 | O |
| ATOM | 5531 | N | GLY | M | 64 | 20.619 | 14.000 | −9.988 | 1.00 | 18.66 | N |
| ATOM | 5532 | CA | GLY | M | 64 | 20.707 | 14.278 | −11.395 | 1.00 | 19.48 | C |
| ATOM | 5533 | C | GLY | M | 64 | 22.077 | 13.885 | −11.867 | 1.00 | 20.44 | C |
| ATOM | 5534 | O | GLY | M | 64 | 23.031 | 13.906 | −11.094 | 1.00 | 20.62 | O |
| ATOM | 5535 | N | SER | M | 65 | 22.168 | 13.504 | −13.135 | 1.00 | 21.38 | N |
| ATOM | 5536 | CA | SER | M | 65 | 23.442 | 13.246 | −13.779 | 1.00 | 22.20 | C |
| ATOM | 5537 | CB | SER | M | 65 | 23.848 | 11.785 | −13.608 | 1.00 | 22.02 | C |
| ATOM | 5538 | OG | SER | M | 65 | 22.874 | 10.951 | −14.196 | 1.00 | 22.75 | O |
| ATOM | 5539 | C | SER | M | 65 | 23.289 | 13.557 | −15.246 | 1.00 | 23.05 | C |
| ATOM | 5540 | O | SER | M | 65 | 22.228 | 13.994 | −15.689 | 1.00 | 23.21 | O |
| ATOM | 5541 | N | GLY | M | 66 | 24.356 | 13.301 | −15.999 | 1.00 | 24.37 | N |
| ATOM | 5542 | CA | GLY | M | 66 | 24.374 | 13.514 | −17.433 | 1.00 | 25.03 | C |
| ATOM | 5543 | C | GLY | M | 66 | 25.467 | 14.491 | −17.788 | 1.00 | 25.89 | C |
| ATOM | 5544 | O | GLY | M | 66 | 26.210 | 14.947 | −16.906 | 1.00 | 25.61 | O |
| ATOM | 5545 | N | SER | M | 67 | 25.564 | 14.810 | −19.082 | 1.00 | 26.50 | N |
| ATOM | 5546 | CA | SER | M | 67 | 26.576 | 15.728 | −19.580 | 1.00 | 27.13 | C |
| ATOM | 5547 | CB | SER | M | 67 | 27.983 | 15.135 | −19.409 | 1.00 | 27.42 | C |
| ATOM | 5548 | OG | SER | M | 67 | 28.077 | 13.872 | −20.045 | 1.00 | 28.37 | O |
| ATOM | 5549 | C | SER | M | 67 | 26.329 | 16.086 | −21.035 | 1.00 | 27.32 | C |
| ATOM | 5550 | O | SER | M | 67 | 25.736 | 15.305 | −21.787 | 1.00 | 27.72 | O |
| ATOM | 5551 | N | GLY | M | 68 | 26.793 | 17.276 | −21.414 | 1.00 | 27.32 | N |
| ATOM | 5552 | CA | GLY | M | 68 | 26.684 | 17.776 | −22.776 | 1.00 | 27.16 | C |
| ATOM | 5553 | C | GLY | M | 68 | 25.265 | 18.059 | −23.208 | 1.00 | 27.31 | C |
| ATOM | 5554 | O | GLY | M | 68 | 24.770 | 19.180 | −23.062 | 1.00 | 27.30 | O |
| ATOM | 5555 | N | THR | M | 69 | 24.614 | 17.031 | −23.741 | 1.00 | 27.45 | N |
| ATOM | 5556 | CA | THR | M | 69 | 23.294 | 17.165 | −24.357 | 1.00 | 27.68 | C |
| ATOM | 5557 | CB | THR | M | 69 | 23.357 | 16.877 | −25.867 | 1.00 | 28.13 | C |
| ATOM | 5558 | OG1 | THR | M | 69 | 23.637 | 15.478 | −26.071 | 1.00 | 28.92 | O |
| ATOM | 5559 | CG2 | THR | M | 69 | 24.421 | 17.759 | −26.570 | 1.00 | 27.77 | C |
| ATOM | 5560 | C | THR | M | 69 | 22.328 | 16.149 | −23.791 | 1.00 | 27.52 | C |
| ATOM | 5561 | O | THR | M | 69 | 21.177 | 16.066 | −24.234 | 1.00 | 27.39 | O |
| ATOM | 5562 | N | ASP | M | 70 | 22.803 | 15.368 | −22.822 | 1.00 | 27.49 | N |
| ATOM | 5563 | CA | ASP | M | 70 | 22.065 | 14.201 | −22.339 | 1.00 | 27.47 | C |
| ATOM | 5564 | CB | ASP | M | 70 | 22.736 | 12.927 | −22.856 | 1.00 | 27.97 | C |
| ATOM | 5565 | CG | ASP | M | 70 | 22.082 | 11.671 | −22.327 | 1.00 | 29.63 | C |
| ATOM | 5566 | OD1 | ASP | M | 70 | 20.979 | 11.333 | −22.802 | 1.00 | 31.13 | O |
| ATOM | 5567 | OD2 | ASP | M | 70 | 22.682 | 11.023 | −21.441 | 1.00 | 31.30 | O |
| ATOM | 5568 | C | ASP | M | 70 | 21.977 | 14.193 | −20.808 | 1.00 | 26.68 | C |
| ATOM | 5569 | O | ASP | M | 70 | 23.012 | 14.267 | −20.129 | 1.00 | 26.39 | O |
| ATOM | 5570 | N | PHE | M | 71 | 20.746 | 14.098 | −20.280 | 1.00 | 25.59 | N |
| ATOM | 5571 | CA | PHE | M | 71 | 20.485 | 14.331 | −18.849 | 1.00 | 24.52 | C |
| ATOM | 5572 | CB | PHE | M | 71 | 20.151 | 15.810 | −18.613 | 1.00 | 24.22 | C |
| ATOM | 5573 | CG | PHE | M | 71 | 21.190 | 16.743 | −19.159 | 1.00 | 22.76 | C |
| ATOM | 5574 | CD1 | PHE | M | 71 | 22.329 | 17.031 | −18.427 | 1.00 | 20.77 | C |
| ATOM | 5575 | CE1 | PHE | M | 71 | 23.316 | 17.870 | −18.944 | 1.00 | 22.56 | C |
| ATOM | 5576 | CZ | PHE | M | 71 | 23.162 | 18.419 | −20.218 | 1.00 | 22.13 | C |
| ATOM | 5577 | CE2 | PHE | M | 71 | 22.021 | 18.118 | −20.964 | 1.00 | 21.92 | C |
| ATOM | 5578 | CD2 | PHE | M | 71 | 21.053 | 17.284 | −20.433 | 1.00 | 21.87 | C |
| ATOM | 5579 | C | PHE | M | 71 | 19.403 | 13.450 | −18.256 | 1.00 | 24.31 | C |
| ATOM | 5580 | O | PHE | M | 71 | 18.417 | 13.131 | −18.921 | 1.00 | 24.15 | O |
| ATOM | 5581 | N | THR | M | 72 | 19.600 | 13.068 | −16.994 | 1.00 | 24.05 | N |
| ATOM | 5582 | CA | THR | M | 72 | 18.674 | 12.177 | −16.288 | 1.00 | 23.98 | C |
| ATOM | 5583 | CB | THR | M | 72 | 19.190 | 10.683 | −16.272 | 1.00 | 23.90 | C |
| ATOM | 5584 | OG1 | THR | M | 72 | 19.664 | 10.303 | −17.571 | 1.00 | 23.14 | O |
| ATOM | 5585 | CG2 | THR | M | 72 | 18.091 | 9.714 | −15.841 | 1.00 | 22.34 | C |
| ATOM | 5586 | C | THR | M | 72 | 18.388 | 12.644 | −14.843 | 1.00 | 24.22 | C |
| ATOM | 5587 | O | THR | M | 72 | 19.296 | 13.036 | −14.104 | 1.00 | 24.26 | O |
| ATOM | 5588 | N | LEU | M | 73 | 17.122 | 12.593 | −14.455 | 1.00 | 24.27 | N |
| ATOM | 5589 | CA | LEU | M | 73 | 16.731 | 12.800 | −13.071 | 1.00 | 24.86 | C |
| ATOM | 5590 | CB | LEU | M | 73 | 15.608 | 13.833 | −12.975 | 1.00 | 24.30 | C |
| ATOM | 5591 | CG | LEU | M | 73 | 14.970 | 14.037 | −11.605 | 1.00 | 23.61 | C |
| ATOM | 5592 | CD1 | LEU | M | 73 | 15.909 | 14.790 | −10.711 | 1.00 | 23.71 | C |
| ATOM | 5593 | CD2 | LEU | M | 73 | 13.669 | 14.788 | −11.733 | 1.00 | 23.53 | C |
| ATOM | 5594 | C | LEU | M | 73 | 16.277 | 11.463 | −12.463 | 1.00 | 25.77 | C |
| ATOM | 5595 | O | LEU | M | 73 | 15.415 | 10.773 | −13.019 | 1.00 | 25.69 | O |
| ATOM | 5596 | N | LYS | M | 74 | 16.856 | 11.093 | −11.327 | 1.00 | 26.39 | N |
| ATOM | 5597 | CA | LYS | M | 74 | 16.422 | 9.864 | −10.678 | 1.00 | 27.24 | C |
| ATOM | 5598 | CB | LYS | M | 74 | 17.570 | 8.840 | −10.584 | 1.00 | 27.38 | C |
| ATOM | 5599 | CG | LYS | M | 74 | 17.905 | 8.217 | −11.935 | 1.00 | 29.13 | C |
| ATOM | 5600 | CD | LYS | M | 74 | 18.756 | 6.950 | −11.851 | 1.00 | 33.03 | C |
| ATOM | 5601 | CE | LYS | M | 74 | 20.239 | 7.214 | −12.194 | 1.00 | 35.85 | C |
| ATOM | 5602 | NZ | LYS | M | 74 | 20.464 | 7.792 | −13.564 | 1.00 | 35.51 | N |
| ATOM | 5603 | C | LYS | M | 74 | 15.774 | 10.131 | −9.325 | 1.00 | 27.26 | C |
| ATOM | 5604 | O | LYS | M | 74 | 16.271 | 10.928 | −8.523 | 1.00 | 27.01 | O |
| ATOM | 5605 | N | ILE | M | 75 | 14.628 | 9.503 | −9.110 | 1.00 | 27.48 | N |
| ATOM | 5606 | CA | ILE | M | 75 | 14.059 | 9.423 | −7.780 | 1.00 | 28.21 | C |
| ATOM | 5607 | CB | ILE | M | 75 | 12.559 | 9.796 | −7.757 | 1.00 | 27.79 | C |
| ATOM | 5608 | CG1 | ILE | M | 75 | 12.331 | 11.077 | −8.559 | 1.00 | 27.47 | C |
| ATOM | 5609 | CD1 | ILE | M | 75 | 10.881 | 11.345 | −8.958 | 1.00 | 27.80 | C |
| ATOM | 5610 | CG2 | ILE | M | 75 | 12.075 | 9.956 | −6.313 | 1.00 | 26.57 | C |
| ATOM | 5611 | C | ILE | M | 75 | 14.284 | 7.977 | −7.353 | 1.00 | 29.00 | C |
| ATOM | 5612 | O | ILE | M | 75 | 13.795 | 7.060 | −8.014 | 1.00 | 29.16 | O |
| ATOM | 5613 | N | SER | M | 76 | 15.054 | 7.776 | −6.285 | 1.00 | 29.70 | N |
| ATOM | 5614 | CA | SER | M | 76 | 15.406 | 6.427 | −5.853 | 1.00 | 31.02 | C |
| ATOM | 5615 | CB | SER | M | 76 | 16.537 | 6.439 | −4.826 | 1.00 | 30.61 | C |
| ATOM | 5616 | OG | SER | M | 76 | 16.237 | 7.296 | −3.740 | 1.00 | 31.69 | O |
| ATOM | 5617 | C | SER | M | 76 | 14.199 | 5.664 | −5.312 | 1.00 | 32.07 | C |
| ATOM | 5618 | O | SER | M | 76 | 14.075 | 4.465 | −5.557 | 1.00 | 32.24 | O |
| ATOM | 5619 | N | ARG | M | 77 | 13.323 | 6.361 | −4.582 | 1.00 | 33.15 | N |
| ATOM | 5620 | CA | ARG | M | 77 | 12.077 | 5.780 | −4.067 | 1.00 | 34.22 | C |
| ATOM | 5621 | CB | ARG | M | 77 | 12.291 | 5.090 | −2.706 | 1.00 | 34.79 | C |
| ATOM | 5622 | CG | ARG | M | 77 | 13.107 | 3.784 | −2.775 | 1.00 | 39.07 | C |
| ATOM | 5623 | CD | ARG | M | 77 | 13.602 | 3.311 | −1.389 | 1.00 | 45.03 | C |
| ATOM | 5624 | NE | ARG | M | 77 | 12.591 | 2.535 | −0.670 | 1.00 | 49.11 | N |
| ATOM | 5625 | CZ | ARG | M | 77 | 12.632 | 2.265 | 0.636 | 1.00 | 52.25 | C |
| ATOM | 5626 | NH1 | ARG | M | 77 | 13.639 | 2.712 | 1.394 | 1.00 | 52.76 | N |
| ATOM | 5627 | NH2 | ARG | M | 77 | 11.655 | 1.551 | 1.191 | 1.00 | 52.98 | N |
| ATOM | 5628 | C | ARG | M | 77 | 10.955 | 6.819 | −3.978 | 1.00 | 33.76 | C |
| ATOM | 5629 | O | ARG | M | 77 | 10.943 | 7.686 | −3.093 | 1.00 | 33.59 | O |
| ATOM | 5630 | N | VAL | M | 78 | 10.000 | 6.696 | −4.889 | 1.00 | 33.33 | N |
| ATOM | 5631 | CA | VAL | M | 78 | 8.912 | 7.659 | −5.017 | 1.00 | 33.09 | C |
| ATOM | 5632 | CB | VAL | M | 78 | 8.149 | 7.430 | −6.329 | 1.00 | 32.87 | C |
| ATOM | 5633 | CG1 | VAL | M | 78 | 6.842 | 8.208 | −6.357 | 1.00 | 32.19 | C |
| ATOM | 5634 | CG2 | VAL | M | 78 | 9.040 | 7.799 | −7.511 | 1.00 | 31.95 | C |
| ATOM | 5635 | C | VAL | M | 78 | 7.952 | 7.689 | −3.817 | 1.00 | 33.63 | C |
| ATOM | 5636 | O | VAL | M | 78 | 7.404 | 6.648 | −3.407 | 1.00 | 33.88 | O |
| ATOM | 5637 | N | GLU | M | 79 | 7.771 | 8.891 | −3.264 | 1.00 | 33.29 | N |
| ATOM | 5638 | CA | GLU | M | 79 | 6.801 | 9.146 | −2.201 | 1.00 | 33.34 | C |
| ATOM | 5639 | CB | GLU | M | 79 | 7.419 | 10.006 | −1.105 | 1.00 | 33.53 | C |
| ATOM | 5640 | CG | GLU | M | 79 | 8.606 | 9.386 | −0.407 | 1.00 | 36.80 | C |
| ATOM | 5641 | CD | GLU | M | 79 | 8.586 | 9.654 | 1.081 | 1.00 | 41.57 | C |
| ATOM | 5642 | OE1 | GLU | M | 79 | 8.793 | 10.831 | 1.480 | 1.00 | 43.07 | O |
| ATOM | 5643 | OE2 | GLU | M | 79 | 8.349 | 8.686 | 1.849 | 1.00 | 43.10 | O |
| ATOM | 5644 | C | GLU | M | 79 | 5.587 | 9.864 | −2.763 | 1.00 | 32.83 | C |
| ATOM | 5645 | O | GLU | M | 79 | 5.624 | 10.375 | −3.875 | 1.00 | 33.01 | O |
| ATOM | 5646 | N | ALA | M | 80 | 4.520 | 9.931 | −1.979 | 1.00 | 32.36 | N |
| ATOM | 5647 | CA | ALA | M | 80 | 3.284 | 10.575 | −2.418 | 1.00 | 32.06 | C |
| ATOM | 5648 | CB | ALA | M | 80 | 2.178 | 10.401 | −1.355 | 1.00 | 31.77 | C |
| ATOM | 5649 | C | ALA | M | 80 | 3.464 | 12.059 | −2.794 | 1.00 | 31.69 | C |
| ATOM | 5650 | O | ALA | M | 80 | 2.944 | 12.505 | −3.832 | 1.00 | 31.71 | O |
| ATOM | 5651 | N | GLU | M | 81 | 4.200 | 12.802 | −1.961 | 1.00 | 30.90 | N |
| ATOM | 5652 | CA | GLU | M | 81 | 4.465 | 14.236 | −2.172 | 1.00 | 30.76 | C |
| ATOM | 5653 | CB | GLU | M | 81 | 5.286 | 14.829 | −1.012 | 1.00 | 31.21 | C |
| ATOM | 5654 | CG | GLU | M | 81 | 4.934 | 14.310 | 0.385 | 1.00 | 34.55 | C |
| ATOM | 5655 | CD | GLU | M | 81 | 5.803 | 13.108 | 0.805 | 1.00 | 38.22 | C |
| ATOM | 5656 | OE1 | GLU | M | 81 | 6.951 | 13.338 | 1.292 | 1.00 | 38.26 | O |
| ATOM | 5657 | OE2 | GLU | M | 81 | 5.330 | 11.943 | 0.644 | 1.00 | 36.96 | O |
| ATOM | 5658 | C | GLU | M | 81 | 5.191 | 14.546 | −3.490 | 1.00 | 29.71 | C |
| ATOM | 5659 | O | GLU | M | 81 | 5.253 | 15.707 | −3.896 | 1.00 | 29.81 | O |
| ATOM | 5660 | N | ASP | M | 82 | 5.728 | 13.510 | −4.141 | 1.00 | 28.48 | N |
| ATOM | 5661 | CA | ASP | M | 82 | 6.585 | 13.637 | −5.330 | 1.00 | 27.39 | C |
| ATOM | 5662 | CB | ASP | M | 82 | 7.551 | 12.451 | −5.409 | 1.00 | 27.45 | C |
| ATOM | 5663 | CG | ASP | M | 82 | 8.584 | 12.463 | −4.309 | 1.00 | 27.30 | C |
| ATOM | 5664 | OD1 | ASP | M | 82 | 8.922 | 13.552 | −3.794 | 1.00 | 27.68 | O |
| ATOM | 5665 | OD2 | ASP | M | 82 | 9.074 | 11.369 | −3.970 | 1.00 | 28.39 | O |
| ATOM | 5666 | C | ASP | M | 82 | 5.815 | 13.722 | −6.638 | 1.00 | 26.64 | C |
| ATOM | 5667 | O | ASP | M | 82 | 6.391 | 13.872 | −7.716 | 1.00 | 26.58 | O |
| ATOM | 5668 | N | VAL | M | 83 | 4.508 | 13.598 | −6.528 | 1.00 | 25.79 | N |
| ATOM | 5669 | CA | VAL | M | 83 | 3.594 | 13.737 | −7.642 | 1.00 | 25.13 | C |
| ATOM | 5670 | CB | VAL | M | 83 | 2.217 | 13.265 | −7.152 | 1.00 | 24.87 | C |
| ATOM | 5671 | CG1 | VAL | M | 83 | 1.090 | 14.064 | −7.746 | 1.00 | 24.42 | C |
| ATOM | 5672 | CG2 | VAL | M | 83 | 2.078 | 11.770 | −7.404 | 1.00 | 25.39 | C |
| ATOM | 5673 | C | VAL | M | 83 | 3.547 | 15.180 | −8.193 | 1.00 | 24.73 | C |
| ATOM | 5674 | O | VAL | M | 83 | 3.562 | 16.144 | −7.419 | 1.00 | 24.89 | O |
| ATOM | 5675 | N | GLY | M | 84 | 3.492 | 15.308 | −9.524 | 1.00 | 24.08 | N |
| ATOM | 5676 | CA | GLY | M | 84 | 3.313 | 16.596 | −10.227 | 1.00 | 22.69 | C |
| ATOM | 5677 | C | GLY | M | 84 | 3.939 | 16.551 | −11.614 | 1.00 | 22.15 | C |
| ATOM | 5678 | O | GLY | M | 84 | 4.135 | 15.464 | −12.171 | 1.00 | 22.11 | O |
| ATOM | 5679 | N | VAL | M | 85 | 4.273 | 17.713 | −12.182 | 1.00 | 21.42 | N |
| ATOM | 5680 | CA | VAL | M | 85 | 4.971 | 17.730 | −13.482 | 1.00 | 20.67 | C |
| ATOM | 5681 | CB | VAL | M | 85 | 4.254 | 18.597 | −14.590 | 1.00 | 20.74 | C |
| ATOM | 5682 | CG1 | VAL | M | 85 | 4.979 | 18.474 | −15.921 | 1.00 | 19.05 | C |
| ATOM | 5683 | CG2 | VAL | M | 85 | 2.782 | 18.195 | −14.754 | 1.00 | 20.21 | C |
| ATOM | 5684 | C | VAL | M | 85 | 6.424 | 18.155 | −13.317 | 1.00 | 20.28 | C |
| ATOM | 5685 | O | VAL | M | 85 | 6.704 | 19.189 | −12.721 | 1.00 | 20.56 | O |
| ATOM | 5686 | N | TYR | M | 86 | 7.329 | 17.346 | −13.860 | 1.00 | 19.57 | N |
| ATOM | 5687 | CA | TYR | M | 86 | 8.747 | 17.592 | −13.795 | 1.00 | 19.24 | C |
| ATOM | 5688 | CB | TYR | M | 86 | 9.479 | 16.281 | −13.556 | 1.00 | 18.99 | C |
| ATOM | 5689 | CG | TYR | M | 86 | 9.298 | 15.710 | −12.169 | 1.00 | 19.67 | C |
| ATOM | 5690 | CD1 | TYR | M | 86 | 8.140 | 15.010 | −11.822 | 1.00 | 20.81 | C |
| ATOM | 5691 | CE1 | TYR | M | 86 | 7.971 | 14.482 | −10.545 | 1.00 | 20.69 | C |
| ATOM | 5692 | CZ | TYR | M | 86 | 8.974 | 14.649 | −9.606 | 1.00 | 21.42 | C |
| ATOM | 5693 | OH | TYR | M | 86 | 8.817 | 14.151 | −8.342 | 1.00 | 19.69 | O |
| ATOM | 5694 | CE2 | TYR | M | 86 | 10.137 | 15.332 | −9.931 | 1.00 | 20.83 | C |
| ATOM | 5695 | CD2 | TYR | M | 86 | 10.287 | 15.857 | −11.206 | 1.00 | 19.95 | C |
| ATOM | 5696 | C | TYR | M | 86 | 9.198 | 18.186 | −15.120 | 1.00 | 19.46 | C |
| ATOM | 5697 | O | TYR | M | 86 | 8.861 | 17.650 | −16.179 | 1.00 | 19.86 | O |
| ATOM | 5698 | N | PHE | M | 87 | 9.948 | 19.287 | −15.066 | 1.00 | 19.17 | N |
| ATOM | 5699 | CA | PHE | M | 87 | 10.465 | 19.942 | −16.263 | 1.00 | 18.88 | C |
| ATOM | 5700 | CB | PHE | M | 87 | 9.930 | 21.375 | −16.392 | 1.00 | 18.85 | C |
| ATOM | 5701 | CG | PHE | M | 87 | 8.426 | 21.501 | −16.469 | 1.00 | 17.01 | C |
| ATOM | 5702 | CD1 | PHE | M | 87 | 7.765 | 21.412 | −17.692 | 1.00 | 16.88 | C |
| ATOM | 5703 | CE1 | PHE | M | 87 | 6.377 | 21.556 | −17.776 | 1.00 | 14.54 | C |
| ATOM | 5704 | CZ | PHE | M | 87 | 5.641 | 21.809 | −16.629 | 1.00 | 14.98 | C |
| ATOM | 5705 | CE2 | PHE | M | 87 | 6.285 | 21.903 | −15.405 | 1.00 | 15.26 | C |
| ATOM | 5706 | CD2 | PHE | M | 87 | 7.682 | 21.771 | −15.334 | 1.00 | 15.37 | C |
| ATOM | 5707 | C | PHE | M | 87 | 11.973 | 20.064 | −16.163 | 1.00 | 19.40 | C |
| ATOM | 5708 | O | PHE | M | 87 | 12.498 | 20.406 | −15.099 | 1.00 | 20.15 | O |
| ATOM | 5709 | N | CYS | M | 88 | 12.689 | 19.805 | −17.251 | 1.00 | 19.84 | N |
| ATOM | 5710 | CA | CYS | M | 88 | 14.090 | 20.210 | −17.291 | 1.00 | 20.25 | C |
| ATOM | 5711 | CB | CYS | M | 88 | 14.952 | 19.312 | −18.189 | 1.00 | 20.93 | C |
| ATOM | 5712 | SG | CYS | M | 88 | 14.348 | 19.027 | −19.871 | 1.00 | 25.00 | S |
| ATOM | 5713 | C | CYS | M | 88 | 14.132 | 21.664 | −17.719 | 1.00 | 19.63 | C |
| ATOM | 5714 | O | CYS | M | 88 | 13.170 | 22.177 | −18.284 | 1.00 | 19.27 | O |
| ATOM | 5715 | N | SER | M | 89 | 15.231 | 22.337 | −17.408 | 1.00 | 19.70 | N |
| ATOM | 5716 | CA | SER | M | 89 | 15.366 | 23.754 | −17.708 | 1.00 | 19.85 | C |
| ATOM | 5717 | CB | SER | M | 89 | 14.964 | 24.585 | −16.484 | 1.00 | 19.72 | C |
| ATOM | 5718 | OG | SER | M | 89 | 15.270 | 25.954 | −16.673 | 1.00 | 19.39 | O |
| ATOM | 5719 | C | SER | M | 89 | 16.799 | 24.038 | −18.094 | 1.00 | 19.78 | C |
| ATOM | 5720 | O | SER | M | 89 | 17.699 | 23.479 | −17.498 | 1.00 | 20.19 | O |
| ATOM | 5721 | N | GLN | M | 90 | 17.020 | 24.879 | −19.104 | 1.00 | 20.05 | N |
| ATOM | 5722 | CA | GLN | M | 90 | 18.390 | 25.296 | −19.443 | 1.00 | 19.51 | C |
| ATOM | 5723 | CB | GLN | M | 90 | 18.851 | 24.765 | −20.815 | 1.00 | 19.63 | C |
| ATOM | 5724 | CG | GLN | M | 90 | 18.141 | 25.342 | −22.047 | 1.00 | 19.68 | C |
| ATOM | 5725 | CD | GLN | M | 90 | 18.716 | 26.673 | −22.555 | 1.00 | 20.35 | C |
| ATOM | 5726 | OE1 | GLN | M | 90 | 19.854 | 27.057 | −22.244 | 1.00 | 18.30 | O |
| ATOM | 5727 | NE2 | GLN | M | 90 | 17.913 | 27.380 | −23.351 | 1.00 | 21.15 | N |
| ATOM | 5728 | C | GLN | M | 90 | 18.551 | 26.799 | −19.365 | 1.00 | 19.63 | C |
| ATOM | 5729 | O | GLN | M | 90 | 17.654 | 27.554 | −19.732 | 1.00 | 19.10 | O |
| ATOM | 5730 | N | SER | M | 91 | 19.702 | 27.230 | −18.870 | 1.00 | 20.23 | N |
| ATOM | 5731 | CA | SER | M | 91 | 19.992 | 28.651 | −18.804 | 1.00 | 21.05 | C |
| ATOM | 5732 | CB | SER | M | 91 | 19.816 | 29.198 | −17.380 | 1.00 | 20.48 | C |
| ATOM | 5733 | OG | SER | M | 91 | 20.686 | 28.560 | −16.466 | 1.00 | 20.42 | O |
| ATOM | 5734 | C | SER | M | 91 | 21.379 | 28.931 | −19.366 | 1.00 | 21.71 | C |
| ATOM | 5735 | O | SER | M | 91 | 21.986 | 29.965 | −19.070 | 1.00 | 21.71 | O |
| ATOM | 5736 | N | THR | M | 92 | 21.869 | 28.006 | −20.188 | 1.00 | 22.72 | N |
| ATOM | 5737 | CA | THR | M | 92 | 23.169 | 28.183 | −20.823 | 1.00 | 24.07 | C |
| ATOM | 5738 | CB | THR | M | 92 | 23.778 | 26.841 | −21.319 | 1.00 | 24.08 | C |
| ATOM | 5739 | OG1 | THR | M | 92 | 24.014 | 25.986 | −20.193 | 1.00 | 24.62 | O |
| ATOM | 5740 | CG2 | THR | M | 92 | 25.129 | 27.066 | −22.050 | 1.00 | 24.35 | C |
| ATOM | 5741 | C | THR | M | 92 | 23.041 | 29.198 | −21.953 | 1.00 | 24.65 | C |
| ATOM | 5742 | O | THR | M | 92 | 23.874 | 30.082 | −22.093 | 1.00 | 24.58 | O |
| ATOM | 5743 | N | HIS | M | 93 | 21.960 | 29.083 | −22.714 | 1.00 | 25.75 | N |
| ATOM | 5744 | CA | HIS | M | 93 | 21.772 | 29.843 | −23.943 | 1.00 | 26.63 | C |
| ATOM | 5745 | CB | HIS | M | 93 | 21.798 | 28.898 | −25.160 | 1.00 | 27.22 | C |
| ATOM | 5746 | CG | HIS | M | 93 | 23.050 | 28.077 | −25.288 | 1.00 | 29.76 | C |
| ATOM | 5747 | ND1 | HIS | M | 93 | 24.251 | 28.603 | −25.723 | 1.00 | 32.30 | N |
| ATOM | 5748 | CE1 | HIS | M | 93 | 25.166 | 27.649 | −25.753 | 1.00 | 33.56 | C |
| ATOM | 5749 | NE2 | HIS | M | 93 | 24.601 | 26.518 | −25.366 | 1.00 | 33.28 | N |
| ATOM | 5750 | CD2 | HIS | M | 93 | 23.275 | 26.756 | −25.077 | 1.00 | 32.14 | C |
| ATOM | 5751 | C | HIS | M | 93 | 20.430 | 30.557 | −23.924 | 1.00 | 26.48 | C |
| ATOM | 5752 | O | HIS | M | 93 | 19.411 | 29.979 | −23.524 | 1.00 | 26.04 | O |
| ATOM | 5753 | N | PHE | M | 94 | 20.440 | 31.811 | −24.373 | 1.00 | 26.88 | N |
| ATOM | 5754 | CA | PHE | M | 94 | 19.214 | 32.580 | −24.596 | 1.00 | 27.26 | C |
| ATOM | 5755 | CB | PHE | M | 94 | 19.489 | 34.088 | −24.547 | 1.00 | 27.29 | C |
| ATOM | 5756 | CG | PHE | M | 94 | 19.582 | 34.645 | −23.157 | 1.00 | 27.98 | C |
| ATOM | 5757 | CD1 | PHE | M | 94 | 18.435 | 34.780 | −22.368 | 1.00 | 27.25 | C |
| ATOM | 5758 | CE1 | PHE | M | 94 | 18.507 | 35.289 | −21.077 | 1.00 | 26.53 | C |
| ATOM | 5759 | CZ | PHE | M | 94 | 19.734 | 35.683 | −20.555 | 1.00 | 27.55 | C |
| ATOM | 5760 | CE2 | PHE | M | 94 | 20.898 | 35.565 | −21.331 | 1.00 | 29.13 | C |
| ATOM | 5761 | CD2 | PHE | M | 94 | 20.814 | 35.049 | −22.635 | 1.00 | 29.02 | C |
| ATOM | 5762 | C | PHE | M | 94 | 18.639 | 32.229 | −25.957 | 1.00 | 27.14 | C |
| ATOM | 5763 | O | PHE | M | 94 | 19.397 | 32.016 | −26.896 | 1.00 | 27.71 | O |
| ATOM | 5764 | N | PRO | M | 95 | 17.301 | 32.165 | −26.078 | 1.00 | 27.07 | N |
| ATOM | 5765 | CA | PRO | M | 95 | 16.328 | 32.293 | −24.998 | 1.00 | 26.67 | C |
| ATOM | 5766 | CB | PRO | M | 95 | 15.010 | 32.424 | −25.745 | 1.00 | 26.72 | C |
| ATOM | 5767 | CG | PRO | M | 95 | 15.228 | 31.623 | −27.007 | 1.00 | 27.25 | C |
| ATOM | 5768 | CD | PRO | M | 95 | 16.645 | 31.967 | −27.384 | 1.00 | 27.10 | C |
| ATOM | 5769 | C | PRO | M | 95 | 16.324 | 31.021 | −24.144 | 1.00 | 26.40 | C |
| ATOM | 5770 | O | PRO | M | 95 | 16.570 | 29.926 | −24.661 | 1.00 | 26.27 | O |
| ATOM | 5771 | N | PHE | M | 96 | 16.077 | 31.185 | −22.845 | 1.00 | 25.88 | N |
| ATOM | 5772 | CA | PHE | M | 96 | 15.994 | 30.070 | −21.919 | 1.00 | 24.65 | C |
| ATOM | 5773 | CB | PHE | M | 96 | 15.834 | 30.578 | −20.492 | 1.00 | 24.40 | C |
| ATOM | 5774 | CG | PHE | M | 96 | 17.045 | 31.307 | −19.960 | 1.00 | 23.75 | C |
| ATOM | 5775 | CD1 | PHE | M | 96 | 16.957 | 32.064 | −18.790 | 1.00 | 22.34 | C |
| ATOM | 5776 | CE1 | PHE | M | 96 | 18.071 | 32.744 | −18.292 | 1.00 | 20.49 | C |
| ATOM | 5777 | CZ | PHE | M | 96 | 19.281 | 32.669 | −18.958 | 1.00 | 19.75 | C |
| ATOM | 5778 | CE2 | PHE | M | 96 | 19.384 | 31.922 | −20.129 | 1.00 | 20.10 | C |
| ATOM | 5779 | CD2 | PHE | M | 96 | 18.273 | 31.245 | −20.622 | 1.00 | 21.56 | C |
| ATOM | 5780 | C | PHE | M | 96 | 14.807 | 29.228 | −22.314 | 1.00 | 24.49 | C |
| ATOM | 5781 | O | PHE | M | 96 | 13.784 | 29.756 | −22.770 | 1.00 | 23.76 | O |
| ATOM | 5782 | N | THR | M | 97 | 14.952 | 27.915 | −22.175 | 1.00 | 24.65 | N |
| ATOM | 5783 | CA | THR | M | 97 | 13.911 | 26.987 | −22.625 | 1.00 | 25.01 | C |
| ATOM | 5784 | CB | THR | M | 97 | 14.233 | 26.373 | −24.003 | 1.00 | 24.93 | C |
| ATOM | 5785 | OG1 | THR | M | 97 | 15.558 | 25.828 | −23.982 | 1.00 | 25.85 | O |
| ATOM | 5786 | CG2 | THR | M | 97 | 14.148 | 27.421 | −25.099 | 1.00 | 24.78 | C |
| ATOM | 5787 | C | THR | M | 97 | 13.683 | 25.869 | −21.633 | 1.00 | 25.17 | C |
| ATOM | 5788 | O | THR | M | 97 | 14.507 | 25.625 | −20.746 | 1.00 | 25.59 | O |
| ATOM | 5789 | N | PHE | M | 98 | 12.554 | 25.193 | −21.806 | 1.00 | 25.82 | N |
| ATOM | 5790 | CA | PHE | M | 98 | 12.121 | 24.097 | −20.946 | 1.00 | 26.03 | C |
| ATOM | 5791 | CB | PHE | M | 98 | 10.917 | 24.549 | −20.122 | 1.00 | 26.06 | C |
| ATOM | 5792 | CG | PHE | M | 98 | 11.224 | 25.617 | −19.133 | 1.00 | 25.98 | C |
| ATOM | 5793 | CD1 | PHE | M | 98 | 11.097 | 26.954 | −19.478 | 1.00 | 26.12 | C |
| ATOM | 5794 | CE1 | PHE | M | 98 | 11.367 | 27.955 | −18.548 | 1.00 | 26.12 | C |
| ATOM | 5795 | CZ | PHE | M | 98 | 11.756 | 27.617 | −17.263 | 1.00 | 26.11 | C |
| ATOM | 5796 | CE2 | PHE | M | 98 | 11.878 | 26.282 | −16.907 | 1.00 | 26.21 | C |
| ATOM | 5797 | CD2 | PHE | M | 98 | 11.609 | 25.289 | −17.838 | 1.00 | 26.03 | C |
| ATOM | 5798 | C | PHE | M | 98 | 11.675 | 22.894 | −21.766 | 1.00 | 26.13 | C |
| ATOM | 5799 | O | PHE | M | 98 | 11.125 | 23.054 | −22.855 | 1.00 | 25.92 | O |
| ATOM | 5800 | N | GLY | M | 99 | 11.876 | 21.696 | −21.223 | 1.00 | 26.63 | N |
| ATOM | 5801 | CA | GLY | M | 99 | 11.276 | 20.487 | −21.782 | 1.00 | 27.08 | C |
| ATOM | 5802 | C | GLY | M | 99 | 9.780 | 20.593 | −21.596 | 1.00 | 27.85 | C |
| ATOM | 5803 | O | GLY | M | 99 | 9.318 | 21.420 | −20.814 | 1.00 | 27.93 | O |
| ATOM | 5804 | N | GLN | M | 100 | 9.017 | 19.764 | −22.306 | 1.00 | 28.91 | N |
| ATOM | 5805 | CA | GLN | M | 100 | 7.548 | 19.850 | −22.277 | 1.00 | 29.71 | C |
| ATOM | 5806 | CB | GLN | M | 100 | 6.907 | 19.178 | −23.499 | 1.00 | 30.26 | C |
| ATOM | 5807 | CG | GLN | M | 100 | 7.018 | 20.007 | −24.805 | 1.00 | 33.75 | C |
| ATOM | 5808 | CD | GLN | M | 100 | 6.580 | 21.482 | −24.640 | 1.00 | 37.88 | C |
| ATOM | 5809 | OE1 | GLN | M | 100 | 5.457 | 21.775 | −24.196 | 1.00 | 39.56 | O |
| ATOM | 5810 | NE2 | GLN | M | 100 | 7.470 | 22.406 | −25.006 | 1.00 | 38.00 | N |
| ATOM | 5811 | C | GLN | M | 100 | 6.967 | 19.295 | −20.992 | 1.00 | 29.34 | C |
| ATOM | 5812 | O | GLN | M | 100 | 5.802 | 19.542 | −20.672 | 1.00 | 29.30 | O |
| ATOM | 5813 | N | GLY | M | 101 | 7.786 | 18.547 | −20.257 | 1.00 | 29.01 | N |
| ATOM | 5814 | CA | GLY | M | 101 | 7.400 | 18.106 | −18.928 | 1.00 | 28.48 | C |
| ATOM | 5815 | C | GLY | M | 101 | 6.932 | 16.678 | −18.859 | 1.00 | 28.12 | C |
| ATOM | 5816 | O | GLY | M | 101 | 6.247 | 16.200 | −19.770 | 1.00 | 28.21 | O |
| ATOM | 5817 | N | THR | M | 102 | 7.309 | 16.015 | −17.760 | 1.00 | 27.59 | N |
| ATOM | 5818 | CA | THR | M | 102 | 6.901 | 14.654 | −17.440 | 1.00 | 26.77 | C |
| ATOM | 5819 | CB | THR | M | 102 | 8.110 | 13.825 | −16.983 | 1.00 | 27.02 | C |
| ATOM | 5820 | OG1 | THR | M | 102 | 9.069 | 13.745 | −18.041 | 1.00 | 26.50 | O |
| ATOM | 5821 | CG2 | THR | M | 102 | 7.673 | 12.418 | −16.569 | 1.00 | 27.11 | C |
| ATOM | 5822 | C | THR | M | 102 | 5.923 | 14.674 | −16.280 | 1.00 | 26.29 | C |
| ATOM | 5823 | O | THR | M | 102 | 6.280 | 15.076 | −15.173 | 1.00 | 26.77 | O |
| ATOM | 5824 | N | LYS | M | 103 | 4.700 | 14.224 | −16.522 | 1.00 | 25.44 | N |
| ATOM | 5825 | CA | LYS | M | 103 | 3.724 | 14.083 | −15.453 | 1.00 | 24.88 | C |
| ATOM | 5826 | CB | LYS | M | 103 | 2.297 | 14.221 | −15.982 | 1.00 | 24.70 | C |
| ATOM | 5827 | CG | LYS | M | 103 | 1.243 | 14.323 | −14.887 | 1.00 | 24.34 | C |
| ATOM | 5828 | CD | LYS | M | 103 | −0.129 | 14.627 | −15.470 | 1.00 | 24.37 | C |
| ATOM | 5829 | CE | LYS | M | 103 | −1.104 | 15.101 | −14.390 | 1.00 | 24.72 | C |
| ATOM | 5830 | NZ | LYS | M | 103 | −2.386 | 15.622 | −14.966 | 1.00 | 24.27 | N |
| ATOM | 5831 | C | LYS | M | 103 | 3.902 | 12.753 | −14.718 | 1.00 | 24.73 | C |
| ATOM | 5832 | O | LYS | M | 103 | 3.837 | 11.667 | −15.304 | 1.00 | 24.56 | O |
| ATOM | 5833 | N | LEU | M | 104 | 4.148 | 12.856 | −13.422 | 1.00 | 24.59 | N |
| ATOM | 5834 | CA | LEU | M | 104 | 4.333 | 11.696 | −12.585 | 1.00 | 24.10 | C |
| ATOM | 5835 | CB | LEU | M | 104 | 5.621 | 11.824 | −11.763 | 1.00 | 23.76 | C |
| ATOM | 5836 | CG | LEU | M | 104 | 6.015 | 10.614 | −10.911 | 1.00 | 22.92 | C |
| ATOM | 5837 | CD1 | LEU | M | 104 | 7.506 | 10.533 | −10.744 | 1.00 | 22.59 | C |
| ATOM | 5838 | CD2 | LEU | M | 104 | 5.324 | 10.621 | −9.550 | 1.00 | 21.30 | C |
| ATOM | 5839 | C | LEU | M | 104 | 3.125 | 11.584 | −11.683 | 1.00 | 24.14 | C |
| ATOM | 5840 | O | LEU | M | 104 | 2.741 | 12.555 | −11.043 | 1.00 | 23.82 | O |
| ATOM | 5841 | N | GLU | M | 105 | 2.533 | 10.393 | −11.645 | 1.00 | 24.67 | N |
| ATOM | 5842 | CA | GLU | M | 105 | 1.404 | 10.102 | −10.753 | 1.00 | 25.74 | C |
| ATOM | 5843 | CB | GLU | M | 105 | 0.060 | 10.421 | −11.435 | 1.00 | 26.32 | C |
| ATOM | 5844 | CG | GLU | M | 105 | −0.077 | 9.945 | −12.870 | 1.00 | 29.83 | C |
| ATOM | 5845 | CD | GLU | M | 105 | −0.911 | 10.897 | −13.746 | 1.00 | 34.48 | C |
| ATOM | 5846 | OE1 | GLU | M | 105 | −0.530 | 11.066 | −14.938 | 1.00 | 36.43 | O |
| ATOM | 5847 | OE2 | GLU | M | 105 | −1.931 | 11.460 | −13.252 | 1.00 | 33.31 | O |
| ATOM | 5848 | C | GLU | M | 105 | 1.437 | 8.678 | −10.201 | 1.00 | 25.23 | C |
| ATOM | 5849 | O | GLU | M | 105 | 2.283 | 7.873 | −10.597 | 1.00 | 25.58 | O |
| ATOM | 5850 | N | ILE | M | 106 | 0.523 | 8.377 | −9.283 | 1.00 | 24.76 | N |
| ATOM | 5851 | CA | ILE | M | 106 | 0.490 | 7.089 | −8.599 | 1.00 | 24.58 | C |
| ATOM | 5852 | CB | ILE | M | 106 | −0.420 | 7.152 | −7.344 | 1.00 | 24.99 | C |
| ATOM | 5853 | CG1 | ILE | M | 106 | −0.091 | 8.367 | −6.452 | 1.00 | 25.22 | C |
| ATOM | 5854 | CD1 | ILE | M | 106 | 1.347 | 8.422 | −5.944 | 1.00 | 25.13 | C |
| ATOM | 5855 | CG2 | ILE | M | 106 | −0.374 | 5.828 | −6.567 | 1.00 | 24.90 | C |
| ATOM | 5856 | C | ILE | M | 106 | −0.001 | 5.932 | −9.484 | 1.00 | 24.38 | C |
| ATOM | 5857 | O | ILE | M | 106 | −1.104 | 5.977 | −10.044 | 1.00 | 23.89 | O |
| ATOM | 5858 | N | LYS | M | 107 | 0.811 | 4.887 | −9.598 | 1.00 | 24.33 | N |
| ATOM | 5859 | CA | LYS | M | 107 | 0.312 | 3.633 | −10.160 | 1.00 | 24.16 | C |
| ATOM | 5860 | CB | LYS | M | 107 | 1.404 | 2.802 | −10.837 | 1.00 | 24.76 | C |
| ATOM | 5861 | CG | LYS | M | 107 | 0.869 | 1.477 | −11.431 | 1.00 | 26.48 | C |
| ATOM | 5862 | CD | LYS | M | 107 | 1.591 | 1.079 | −12.709 | 1.00 | 29.53 | C |
| ATOM | 5863 | CE | LYS | M | 107 | 2.951 | 0.441 | −12.421 | 1.00 | 31.95 | C |
| ATOM | 5864 | NZ | LYS | M | 107 | 2.792 | −0.942 | −11.856 | 1.00 | 32.63 | N |
| ATOM | 5865 | C | LYS | M | 107 | −0.382 | 2.815 | −9.096 | 1.00 | 23.60 | C |
| ATOM | 5866 | O | LYS | M | 107 | 0.090 | 2.683 | −7.955 | 1.00 | 23.42 | O |
| ATOM | 5867 | N | ARG | M | 108 | −1.524 | 2.273 | −9.488 | 1.00 | 23.28 | N |
| ATOM | 5868 | CA | ARG | M | 108 | −2.295 | 1.382 | −8.644 | 1.00 | 22.66 | C |
| ATOM | 5869 | CB | ARG | M | 108 | −3.371 | 2.163 | −7.903 | 1.00 | 21.98 | C |
| ATOM | 5870 | CG | ARG | M | 108 | −4.291 | 2.944 | −8.792 | 1.00 | 21.15 | C |
| ATOM | 5871 | CD | ARG | M | 108 | −5.677 | 3.000 | −8.186 | 1.00 | 20.18 | C |
| ATOM | 5872 | NE | ARG | M | 108 | −6.322 | 1.687 | −8.176 | 1.00 | 19.41 | N |
| ATOM | 5873 | CZ | ARG | M | 108 | −7.342 | 1.348 | −7.388 | 1.00 | 18.82 | C |
| ATOM | 5874 | NH1 | ARG | M | 108 | −7.854 | 0.125 | −7.462 | 1.00 | 18.68 | N |
| ATOM | 5875 | NH2 | ARG | M | 108 | −7.842 | 2.218 | −6.518 | 1.00 | 16.40 | N |
| ATOM | 5876 | C | ARG | M | 108 | −2.881 | 0.258 | −9.507 | 1.00 | 22.98 | C |
| ATOM | 5877 | O | ARG | M | 108 | −2.581 | 0.170 | −10.706 | 1.00 | 22.84 | O |
| ATOM | 5878 | N | THR | M | 109 | −3.690 | −0.610 | −8.901 | 1.00 | 23.04 | N |
| ATOM | 5879 | CA | THR | M | 109 | −4.343 | −1.673 | −9.649 | 1.00 | 22.96 | C |
| ATOM | 5880 | CB | THR | M | 109 | −4.973 | −2.754 | −8.736 | 1.00 | 22.95 | C |
| ATOM | 5881 | OG1 | THR | M | 109 | −5.910 | −2.151 | −7.840 | 1.00 | 22.41 | O |
| ATOM | 5882 | CG2 | THR | M | 109 | −3.892 | −3.517 | −7.951 | 1.00 | 22.71 | C |
| ATOM | 5883 | C | THR | M | 109 | −5.431 | −1.087 | −10.521 | 1.00 | 23.16 | C |
| ATOM | 5884 | O | THR | M | 109 | −6.039 | −0.067 | −10.173 | 1.00 | 23.60 | O |
| ATOM | 5885 | N | VAL | M | 110 | −5.670 | −1.731 | −11.655 | 1.00 | 22.87 | N |
| ATOM | 5886 | CA | VAL | M | 110 | −6.756 | −1.355 | −12.538 | 1.00 | 22.88 | C |
| ATOM | 5887 | CB | VAL | M | 110 | −6.766 | −2.276 | −13.768 | 1.00 | 22.76 | C |
| ATOM | 5888 | CG1 | VAL | M | 110 | −8.082 | −2.176 | −14.541 | 1.00 | 21.46 | C |
| ATOM | 5889 | CG2 | VAL | M | 110 | −5.566 | −1.965 | −14.663 | 1.00 | 22.35 | C |
| ATOM | 5890 | C | VAL | M | 110 | −8.098 | −1.427 | −11.805 | 1.00 | 23.48 | C |
| ATOM | 5891 | O | VAL | M | 110 | −8.371 | −2.388 | −11.089 | 1.00 | 23.63 | O |
| ATOM | 5892 | N | ALA | M | 111 | −8.919 | −0.394 | −11.960 | 1.00 | 23.91 | N |
| ATOM | 5893 | CA | ALA | M | 111 | −10.285 | −0.438 | −11.462 | 1.00 | 24.52 | C |
| ATOM | 5894 | CB | ALA | M | 111 | −10.409 | 0.288 | −10.157 | 1.00 | 24.12 | C |
| ATOM | 5895 | C | ALA | M | 111 | −11.258 | 0.130 | −12.482 | 1.00 | 25.66 | C |
| ATOM | 5896 | O | ALA | M | 111 | −10.975 | 1.147 | −13.139 | 1.00 | 26.10 | O |
| ATOM | 5897 | N | ALA | M | 112 | −12.403 | −0.539 | −12.593 | 1.00 | 26.59 | N |
| ATOM | 5898 | CA | ALA | M | 112 | −13.466 | −0.191 | −13.528 | 1.00 | 27.51 | C |
| ATOM | 5899 | CB | ALA | M | 112 | −14.345 | −1.404 | −13.776 | 1.00 | 27.16 | C |
| ATOM | 5900 | C | ALA | M | 112 | −14.312 | 0.961 | −12.997 | 1.00 | 28.32 | C |
| ATOM | 5901 | O | ALA | M | 112 | −14.483 | 1.098 | −11.783 | 1.00 | 29.09 | O |
| ATOM | 5902 | N | PRO | M | 113 | −14.858 | 1.792 | −13.901 | 1.00 | 28.81 | N |
| ATOM | 5903 | CA | PRO | M | 113 | −15.727 | 2.874 | −13.442 | 1.00 | 28.85 | C |
| ATOM | 5904 | CB | PRO | M | 113 | −15.922 | 3.731 | −14.700 | 1.00 | 28.66 | C |
| ATOM | 5905 | CG | PRO | M | 113 | −15.694 | 2.795 | −15.841 | 1.00 | 29.26 | C |
| ATOM | 5906 | CD | PRO | M | 113 | −14.637 | 1.832 | −15.363 | 1.00 | 29.03 | C |
| ATOM | 5907 | C | PRO | M | 113 | −17.082 | 2.378 | −12.969 | 1.00 | 29.07 | C |
| ATOM | 5908 | O | PRO | M | 113 | −17.492 | 1.282 | −13.319 | 1.00 | 29.20 | O |
| ATOM | 5909 | N | SER | M | 114 | −17.754 | 3.185 | −12.157 | 1.00 | 29.08 | N |
| ATOM | 5910 | CA | SER | M | 114 | −19.180 | 3.087 | −12.019 | 1.00 | 29.08 | C |
| ATOM | 5911 | CB | SER | M | 114 | −19.611 | 3.375 | −10.593 | 1.00 | 29.19 | C |
| ATOM | 5912 | OG | SER | M | 114 | −19.086 | 2.384 | −9.732 | 1.00 | 30.22 | O |
| ATOM | 5913 | C | SER | M | 114 | −19.718 | 4.125 | −12.983 | 1.00 | 29.35 | C |
| ATOM | 5914 | O | SER | M | 114 | −19.318 | 5.294 | −12.947 | 1.00 | 29.03 | O |
| ATOM | 5915 | N | VAL | M | 115 | −20.591 | 3.675 | −13.878 | 1.00 | 29.51 | N |
| ATOM | 5916 | CA | VAL | M | 115 | −21.134 | 4.518 | −14.923 | 1.00 | 29.22 | C |
| ATOM | 5917 | CB | VAL | M | 115 | −21.081 | 3.816 | −16.300 | 1.00 | 29.12 | C |
| ATOM | 5918 | CG1 | VAL | M | 115 | −21.595 | 4.740 | −17.414 | 1.00 | 29.16 | C |
| ATOM | 5919 | CG2 | VAL | M | 115 | −19.681 | 3.366 | −16.607 | 1.00 | 27.86 | C |
| ATOM | 5920 | C | VAL | M | 115 | −22.563 | 4.881 | −14.569 | 1.00 | 29.69 | C |
| ATOM | 5921 | O | VAL | M | 115 | −23.363 | 4.011 | −14.215 | 1.00 | 29.79 | O |
| ATOM | 5922 | N | PHE | M | 116 | −22.862 | 6.176 | −14.643 | 1.00 | 30.20 | N |
| ATOM | 5923 | CA | PHE | M | 116 | −24.202 | 6.703 | −14.401 | 1.00 | 30.66 | C |
| ATOM | 5924 | CB | PHE | M | 116 | −24.250 | 7.532 | −13.121 | 1.00 | 30.69 | C |
| ATOM | 5925 | CG | PHE | M | 116 | −23.836 | 6.800 | −11.878 | 1.00 | 29.78 | C |
| ATOM | 5926 | CD1 | PHE | M | 116 | −24.793 | 6.216 | −11.052 | 1.00 | 28.93 | C |
| ATOM | 5927 | CE1 | PHE | M | 116 | −24.416 | 5.558 | −9.872 | 1.00 | 28.03 | C |
| ATOM | 5928 | CZ | PHE | M | 116 | −23.078 | 5.507 | −9.512 | 1.00 | 28.31 | C |
| ATOM | 5929 | CE2 | PHE | M | 116 | −22.112 | 6.109 | −10.331 | 1.00 | 28.66 | C |
| ATOM | 5930 | CD2 | PHE | M | 116 | −22.497 | 6.759 | −11.494 | 1.00 | 28.29 | C |
| ATOM | 5931 | C | PHE | M | 116 | −24.574 | 7.629 | −15.546 | 1.00 | 31.42 | C |
| ATOM | 5932 | O | PHE | M | 116 | −23.706 | 8.289 | −16.134 | 1.00 | 31.39 | O |
| ATOM | 5933 | N | ILE | M | 117 | −25.868 | 7.700 | −15.842 | 1.00 | 32.40 | N |
| ATOM | 5934 | CA | ILE | M | 117 | −26.374 | 8.566 | −16.909 | 1.00 | 33.10 | C |
| ATOM | 5935 | CB | ILE | M | 117 | −26.819 | 7.743 | −18.184 | 1.00 | 33.24 | C |
| ATOM | 5936 | CG1 | ILE | M | 117 | −27.132 | 8.674 | −19.367 | 1.00 | 33.89 | C |
| ATOM | 5937 | CD1 | ILE | M | 117 | −27.120 | 8.000 | −20.744 | 1.00 | 33.79 | C |
| ATOM | 5938 | CG2 | ILE | M | 117 | −27.984 | 6.819 | −17.879 | 1.00 | 32.82 | C |
| ATOM | 5939 | C | ILE | M | 117 | −27.472 | 9.473 | −16.361 | 1.00 | 33.37 | C |
| ATOM | 5940 | O | ILE | M | 117 | −28.281 | 9.053 | −15.527 | 1.00 | 33.37 | O |
| ATOM | 5941 | N | PHE | M | 118 | −27.464 | 10.725 | −16.807 | 1.00 | 33.85 | N |
| ATOM | 5942 | CA | PHE | M | 118 | −28.408 | 11.734 | −16.334 | 1.00 | 34.41 | C |
| ATOM | 5943 | CB | PHE | M | 118 | −27.705 | 12.808 | −15.501 | 1.00 | 33.92 | C |
| ATOM | 5944 | CG | PHE | M | 118 | −27.003 | 12.283 | −14.281 | 1.00 | 32.97 | C |
| ATOM | 5945 | CD1 | PHE | M | 118 | −27.677 | 12.152 | −13.076 | 1.00 | 32.01 | C |
| ATOM | 5946 | CE1 | PHE | M | 118 | −27.026 | 11.674 | −11.944 | 1.00 | 31.44 | C |
| ATOM | 5947 | CZ | PHE | M | 118 | −25.686 | 11.333 | −12.008 | 1.00 | 30.69 | C |
| ATOM | 5948 | CE2 | PHE | M | 118 | −25.002 | 11.467 | −13.197 | 1.00 | 31.41 | C |
| ATOM | 5949 | CD2 | PHE | M | 118 | −25.659 | 11.940 | −14.329 | 1.00 | 31.96 | C |
| ATOM | 5950 | C | PHE | M | 118 | −29.121 | 12.408 | −17.501 | 1.00 | 35.40 | C |
| ATOM | 5951 | O | PHE | M | 118 | −28.482 | 13.006 | −18.371 | 1.00 | 35.05 | O |
| ATOM | 5952 | N | PRO | M | 119 | −30.457 | 12.318 | −17.521 | 1.00 | 36.56 | N |
| ATOM | 5953 | CA | PRO | M | 119 | −31.235 | 13.051 | −18.513 | 1.00 | 37.54 | C |
| ATOM | 5954 | CB | PRO | M | 119 | −32.661 | 12.520 | −18.299 | 1.00 | 37.66 | C |
| ATOM | 5955 | CG | PRO | M | 119 | −32.681 | 12.027 | −16.876 | 1.00 | 37.22 | C |
| ATOM | 5956 | CD | PRO | M | 119 | −31.304 | 11.466 | −16.664 | 1.00 | 36.56 | C |
| ATOM | 5957 | C | PRO | M | 119 | −31.193 | 14.550 | −18.235 | 1.00 | 38.68 | C |
| ATOM | 5958 | O | PRO | M | 119 | −30.850 | 14.954 | −17.119 | 1.00 | 38.98 | O |
| ATOM | 5959 | N | PRO | M | 120 | −31.527 | 15.377 | −19.242 | 1.00 | 39.75 | N |
| ATOM | 5960 | CA | PRO | M | 120 | −31.761 | 16.789 | −18.954 | 1.00 | 40.28 | C |
| ATOM | 5961 | CB | PRO | M | 120 | −32.082 | 17.393 | −20.325 | 1.00 | 40.39 | C |
| ATOM | 5962 | CG | PRO | M | 120 | −32.381 | 16.231 | −21.219 | 1.00 | 40.30 | C |
| ATOM | 5963 | CD | PRO | M | 120 | −31.570 | 15.094 | −20.690 | 1.00 | 39.93 | C |
| ATOM | 5964 | C | PRO | M | 120 | −32.948 | 16.974 | −18.018 | 1.00 | 41.03 | C |
| ATOM | 5965 | O | PRO | M | 120 | −33.961 | 16.274 | −18.131 | 1.00 | 40.93 | O |
| ATOM | 5966 | N | SER | M | 121 | −32.788 | 17.909 | −17.089 | 1.00 | 41.78 | N |
| ATOM | 5967 | CA | SER | M | 121 | −33.843 | 18.327 | −16.193 | 1.00 | 42.34 | C |
| ATOM | 5968 | CB | SER | M | 121 | −33.266 | 19.268 | −15.138 | 1.00 | 42.27 | C |
| ATOM | 5969 | OG | SER | M | 121 | −32.519 | 20.315 | −15.739 | 1.00 | 41.51 | O |
| ATOM | 5970 | C | SER | M | 121 | −34.956 | 19.035 | −16.963 | 1.00 | 43.26 | C |
| ATOM | 5971 | O | SER | M | 121 | −34.724 | 19.599 | −18.041 | 1.00 | 42.95 | O |
| ATOM | 5972 | N | ASP | M | 122 | −36.161 | 18.999 | −16.396 | 1.00 | 44.42 | N |
| ATOM | 5973 | CA | ASP | M | 122 | −37.309 | 19.725 | −16.927 | 1.00 | 45.54 | C |
| ATOM | 5974 | CB | ASP | M | 122 | −38.491 | 19.617 | −15.960 | 1.00 | 45.92 | C |
| ATOM | 5975 | CG | ASP | M | 122 | −39.255 | 18.304 | −16.095 | 1.00 | 47.66 | C |
| ATOM | 5976 | OD1 | ASP | M | 122 | −39.049 | 17.565 | −17.096 | 1.00 | 48.30 | O |
| ATOM | 5977 | OD2 | ASP | M | 122 | −40.078 | 18.027 | −15.187 | 1.00 | 48.80 | O |
| ATOM | 5978 | C | ASP | M | 122 | −36.984 | 21.198 | −17.142 | 1.00 | 45.82 | C |
| ATOM | 5979 | O | ASP | M | 122 | −37.327 | 21.772 | −18.179 | 1.00 | 46.08 | O |
| ATOM | 5980 | N | GLU | M | 123 | −36.311 | 21.785 | −16.152 | 1.00 | 46.16 | N |
| ATOM | 5981 | CA | GLU | M | 123 | −35.982 | 23.208 | −16.118 | 1.00 | 46.46 | C |
| ATOM | 5982 | CB | GLU | M | 123 | −35.327 | 23.550 | −14.781 | 1.00 | 46.64 | C |
| ATOM | 5983 | CG | GLU | M | 123 | −35.285 | 25.032 | −14.464 | 1.00 | 49.12 | C |
| ATOM | 5984 | CD | GLU | M | 123 | −34.755 | 25.328 | −13.069 | 1.00 | 51.97 | C |
| ATOM | 5985 | OE1 | GLU | M | 123 | −34.641 | 24.387 | −12.246 | 1.00 | 53.92 | O |
| ATOM | 5986 | OE2 | GLU | M | 123 | −34.455 | 26.508 | −12.790 | 1.00 | 53.03 | O |
| ATOM | 5987 | C | GLU | M | 123 | −35.100 | 23.666 | −17.281 | 1.00 | 46.04 | C |
| ATOM | 5988 | O | GLU | M | 123 | −35.352 | 24.721 | −17.865 | 1.00 | 45.91 | O |
| ATOM | 5989 | N | GLN | M | 124 | −34.075 | 22.882 | −17.617 | 1.00 | 45.90 | N |
| ATOM | 5990 | CA | GLN | M | 124 | −33.201 | 23.221 | −18.748 | 1.00 | 45.89 | C |
| ATOM | 5991 | CB | GLN | M | 124 | −31.950 | 22.340 | −18.802 | 1.00 | 45.48 | C |
| ATOM | 5992 | CG | GLN | M | 124 | −30.972 | 22.787 | −19.893 | 1.00 | 44.53 | C |
| ATOM | 5993 | CD | GLN | M | 124 | −29.809 | 21.848 | −20.106 | 1.00 | 42.77 | C |
| ATOM | 5994 | OE1 | GLN | M | 124 | −29.898 | 20.654 | −19.845 | 1.00 | 42.75 | O |
| ATOM | 5995 | NE2 | GLN | M | 124 | −28.706 | 22.388 | −20.598 | 1.00 | 42.80 | N |
| ATOM | 5996 | C | GLN | M | 124 | −33.942 | 23.169 | −20.090 | 1.00 | 46.31 | C |
| ATOM | 5997 | O | GLN | M | 124 | −33.663 | 23.961 | −21.000 | 1.00 | 46.24 | O |
| ATOM | 5998 | N | LEU | M | 125 | −34.873 | 22.222 | −20.202 | 1.00 | 46.73 | N |
| ATOM | 5999 | CA | LEU | M | 125 | −35.722 | 22.087 | −21.384 | 1.00 | 46.86 | C |
| ATOM | 6000 | CB | LEU | M | 125 | −36.535 | 20.795 | −21.306 | 1.00 | 46.84 | C |
| ATOM | 6001 | CG | LEU | M | 125 | −35.725 | 19.504 | −21.426 | 1.00 | 46.60 | C |
| ATOM | 6002 | CD1 | LEU | M | 125 | −35.089 | 19.392 | −22.804 | 1.00 | 47.12 | C |
| ATOM | 6003 | CD2 | LEU | M | 125 | −36.593 | 18.291 | −21.139 | 1.00 | 47.00 | C |
| ATOM | 6004 | C | LEU | M | 125 | −36.631 | 23.306 | −21.547 | 1.00 | 47.08 | C |
| ATOM | 6005 | O | LEU | M | 125 | −36.756 | 23.848 | −22.645 | 1.00 | 47.15 | O |
| ATOM | 6006 | N | LYS | M | 126 | −37.224 | 23.749 | −20.439 | 1.00 | 47.34 | N |
| ATOM | 6007 | CA | LYS | M | 126 | −37.966 | 25.017 | −20.383 | 1.00 | 47.76 | C |
| ATOM | 6008 | CB | LYS | M | 126 | −38.473 | 25.307 | −18.951 | 1.00 | 47.94 | C |
| ATOM | 6009 | CG | LYS | M | 126 | −39.643 | 24.432 | −18.495 | 1.00 | 48.90 | C |
| ATOM | 6010 | CD | LYS | M | 126 | −40.997 | 24.917 | −19.062 | 1.00 | 50.59 | C |
| ATOM | 6011 | CE | LYS | M | 126 | −41.903 | 23.750 | −19.482 | 1.00 | 50.80 | C |
| ATOM | 6012 | NZ | LYS | M | 126 | −41.890 | 22.600 | −18.518 | 1.00 | 50.40 | N |
| ATOM | 6013 | C | LYS | M | 126 | −37.192 | 26.236 | −20.906 | 1.00 | 47.45 | C |
| ATOM | 6014 | O | LYS | M | 126 | −37.752 | 27.324 | −20.977 | 1.00 | 48.05 | O |
| ATOM | 6015 | N | SER | M | 127 | −35.922 | 26.064 | −21.269 | 1.00 | 46.99 | N |
| ATOM | 6016 | CA | SER | M | 127 | −35.139 | 27.166 | −21.837 | 1.00 | 46.30 | C |
| ATOM | 6017 | CB | SER | M | 127 | −33.928 | 27.500 | −20.956 | 1.00 | 46.52 | C |
| ATOM | 6018 | OG | SER | M | 127 | −32.873 | 26.570 | −21.142 | 1.00 | 46.44 | O |
| ATOM | 6019 | C | SER | M | 127 | −34.709 | 26.907 | −23.282 | 1.00 | 45.82 | C |
| ATOM | 6020 | O | SER | M | 127 | −34.094 | 27.772 | −23.912 | 1.00 | 45.95 | O |
| ATOM | 6021 | N | GLY | M | 128 | −35.021 | 25.720 | −23.800 | 1.00 | 44.94 | N |
| ATOM | 6022 | CA | GLY | M | 128 | −34.763 | 25.408 | −25.208 | 1.00 | 44.07 | C |
| ATOM | 6023 | C | GLY | M | 128 | −33.466 | 24.684 | −25.546 | 1.00 | 43.41 | C |
| ATOM | 6024 | O | GLY | M | 128 | −33.085 | 24.592 | −26.718 | 1.00 | 43.51 | O |
| ATOM | 6025 | N | THR | M | 129 | −32.781 | 24.161 | −24.537 | 1.00 | 42.53 | N |
| ATOM | 6026 | CA | THR | M | 129 | −31.591 | 23.350 | −24.789 | 1.00 | 41.61 | C |
| ATOM | 6027 | CB | THR | M | 129 | −30.292 | 24.173 | −24.623 | 1.00 | 41.81 | C |
| ATOM | 6028 | OG1 | THR | M | 129 | −29.164 | 23.290 | −24.589 | 1.00 | 42.37 | O |
| ATOM | 6029 | CG2 | THR | M | 129 | −30.332 | 25.024 | −23.350 | 1.00 | 42.05 | C |
| ATOM | 6030 | C | THR | M | 129 | −31.574 | 22.047 | −23.976 | 1.00 | 40.68 | C |
| ATOM | 6031 | O | THR | M | 129 | −32.194 | 21.958 | −22.911 | 1.00 | 40.52 | O |
| ATOM | 6032 | N | ALA | M | 130 | −30.885 | 21.035 | −24.507 | 1.00 | 39.67 | N |
| ATOM | 6033 | CA | ALA | M | 130 | −30.796 | 19.718 | −23.859 | 1.00 | 38.59 | C |
| ATOM | 6034 | CB | ALA | M | 130 | −31.556 | 18.688 | −24.663 | 1.00 | 38.48 | C |
| ATOM | 6035 | C | ALA | M | 130 | −29.367 | 19.234 | −23.609 | 1.00 | 37.82 | C |
| ATOM | 6036 | O | ALA | M | 130 | −28.578 | 19.063 | −24.542 | 1.00 | 37.71 | O |
| ATOM | 6037 | N | SER | M | 131 | −29.043 | 18.997 | −22.342 | 1.00 | 37.03 | N |
| ATOM | 6038 | CA | SER | M | 131 | −27.740 | 18.435 | −21.977 | 1.00 | 36.11 | C |
| ATOM | 6039 | CB | SER | M | 131 | −26.951 | 19.402 | −21.079 | 1.00 | 36.45 | C |
| ATOM | 6040 | OG | SER | M | 131 | −26.417 | 20.517 | −21.801 | 1.00 | 36.70 | O |
| ATOM | 6041 | C | SER | M | 131 | −27.911 | 17.082 | −21.293 | 1.00 | 35.33 | C |
| ATOM | 6042 | O | SER | M | 131 | −28.610 | 16.953 | −20.272 | 1.00 | 35.26 | O |
| ATOM | 6043 | N | VAL | M | 132 | −27.293 | 16.066 | −21.874 | 1.00 | 34.17 | N |
| ATOM | 6044 | CA | VAL | M | 132 | −27.331 | 14.731 | −21.294 | 1.00 | 33.47 | C |
| ATOM | 6045 | CB | VAL | M | 132 | −27.635 | 13.633 | −22.351 | 1.00 | 33.48 | C |
| ATOM | 6046 | CG1 | VAL | M | 132 | −28.004 | 12.335 | −21.673 | 1.00 | 33.64 | C |
| ATOM | 6047 | CG2 | VAL | M | 132 | −28.767 | 14.056 | −23.274 | 1.00 | 34.07 | C |
| ATOM | 6048 | C | VAL | M | 132 | −25.957 | 14.490 | −20.708 | 1.00 | 32.71 | C |
| ATOM | 6049 | O | VAL | M | 132 | −24.956 | 14.693 | −21.398 | 1.00 | 32.95 | O |
| ATOM | 6050 | N | VAL | M | 133 | −25.904 | 14.059 | −19.448 | 1.00 | 31.42 | N |
| ATOM | 6051 | CA | VAL | M | 133 | −24.628 | 13.867 | −18.763 | 1.00 | 30.39 | C |
| ATOM | 6052 | CB | VAL | M | 133 | −24.530 | 14.732 | −17.470 | 1.00 | 30.31 | C |
| ATOM | 6053 | CG1 | VAL | M | 133 | −23.258 | 14.430 | −16.711 | 1.00 | 29.66 | C |
| ATOM | 6054 | CG2 | VAL | M | 133 | −24.566 | 16.217 | −17.817 | 1.00 | 30.47 | C |
| ATOM | 6055 | C | VAL | M | 133 | −24.330 | 12.404 | −18.446 | 1.00 | 29.84 | C |
| ATOM | 6056 | O | VAL | M | 133 | −25.192 | 11.681 | −17.951 | 1.00 | 29.51 | O |
| ATOM | 6057 | N | CYS | M | 134 | −23.094 | 11.992 | −18.720 | 1.00 | 29.31 | N |
| ATOM | 6058 | CA | CYS | M | 134 | −22.615 | 10.668 | −18.356 | 1.00 | 29.01 | C |
| ATOM | 6059 | CB | CYS | M | 134 | −22.223 | 9.881 | −19.605 | 1.00 | 29.47 | C |
| ATOM | 6060 | SG | CYS | M | 134 | −21.927 | 8.126 | −19.296 | 1.00 | 31.57 | S |
| ATOM | 6061 | C | CYS | M | 134 | −21.431 | 10.737 | −17.388 | 1.00 | 28.34 | C |
| ATOM | 6062 | O | CYS | M | 134 | −20.429 | 11.388 | −17.669 | 1.00 | 28.07 | O |
| ATOM | 6063 | N | LEU | M | 135 | −21.552 | 10.040 | −16.257 | 1.00 | 27.80 | N |
| ATOM | 6064 | CA | LEU | M | 135 | −20.485 | 9.964 | −15.259 | 1.00 | 26.92 | C |
| ATOM | 6065 | CB | LEU | M | 135 | −21.051 | 10.238 | −13.865 | 1.00 | 26.56 | C |
| ATOM | 6066 | CG | LEU | M | 135 | −20.110 | 10.076 | −12.666 | 1.00 | 25.98 | C |
| ATOM | 6067 | CD1 | LEU | M | 135 | −18.961 | 11.088 | −12.696 | 1.00 | 24.30 | C |
| ATOM | 6068 | CD2 | LEU | M | 135 | −20.896 | 10.194 | −11.362 | 1.00 | 25.91 | C |
| ATOM | 6069 | C | LEU | M | 135 | −19.722 | 8.631 | −15.248 | 1.00 | 26.65 | C |
| ATOM | 6070 | O | LEU | M | 135 | −20.323 | 7.564 | −15.146 | 1.00 | 26.75 | O |
| ATOM | 6071 | N | LEU | M | 136 | −18.394 | 8.719 | −15.342 | 1.00 | 26.09 | N |
| ATOM | 6072 | CA | LEU | M | 136 | −17.483 | 7.585 | −15.154 | 1.00 | 25.14 | C |
| ATOM | 6073 | CB | LEU | M | 136 | −16.522 | 7.452 | −16.338 | 1.00 | 24.83 | C |
| ATOM | 6074 | CG | LEU | M | 136 | −17.080 | 6.918 | −17.672 | 1.00 | 25.01 | C |
| ATOM | 6075 | CD1 | LEU | M | 136 | −18.066 | 7.877 | −18.342 | 1.00 | 24.62 | C |
| ATOM | 6076 | CD2 | LEU | M | 136 | −15.955 | 6.576 | −18.632 | 1.00 | 23.48 | C |
| ATOM | 6077 | C | LEU | M | 136 | −16.708 | 7.801 | −13.849 | 1.00 | 25.15 | C |
| ATOM | 6078 | O | LEU | M | 136 | −15.736 | 8.568 | −13.789 | 1.00 | 24.62 | O |
| ATOM | 6079 | N | ASN | M | 137 | −17.153 | 7.120 | −12.797 | 1.00 | 25.10 | N |
| ATOM | 6080 | CA | ASN | M | 137 | −16.662 | 7.387 | −11.455 | 1.00 | 24.94 | C |
| ATOM | 6081 | CB | ASN | M | 137 | −17.826 | 7.416 | −10.475 | 1.00 | 25.01 | C |
| ATOM | 6082 | CG | ASN | M | 137 | −17.502 | 8.185 | −9.237 | 1.00 | 26.75 | C |
| ATOM | 6083 | OD1 | ASN | M | 137 | −16.829 | 9.207 | −9.301 | 1.00 | 30.18 | O |
| ATOM | 6084 | ND2 | ASN | M | 137 | −17.976 | 7.712 | −8.095 | 1.00 | 28.40 | N |
| ATOM | 6085 | C | ASN | M | 137 | −15.584 | 6.433 | −10.950 | 1.00 | 24.58 | C |
| ATOM | 6086 | O | ASN | M | 137 | −15.734 | 5.221 | −11.059 | 1.00 | 24.37 | O |
| ATOM | 6087 | N | ASN | M | 138 | −14.508 | 7.006 | −10.395 | 1.00 | 24.26 | N |
| ATOM | 6088 | CA | ASN | M | 138 | −13.439 | 6.276 | −9.696 | 1.00 | 23.78 | C |
| ATOM | 6089 | CB | ASN | M | 138 | −13.869 | 5.826 | −8.284 | 1.00 | 23.69 | C |
| ATOM | 6090 | CG | ASN | M | 138 | −14.259 | 6.992 | −7.378 | 1.00 | 24.95 | C |
| ATOM | 6091 | OD1 | ASN | M | 138 | −14.003 | 8.164 | −7.679 | 1.00 | 27.88 | O |
| ATOM | 6092 | ND2 | ASN | M | 138 | −14.883 | 6.672 | −6.259 | 1.00 | 25.19 | N |
| ATOM | 6093 | C | ASN | M | 138 | −12.877 | 5.098 | −10.472 | 1.00 | 23.44 | C |
| ATOM | 6094 | O | ASN | M | 138 | −13.156 | 3.947 | −10.158 | 1.00 | 23.31 | O |
| ATOM | 6095 | N | PHE | M | 139 | −12.062 | 5.386 | −11.475 | 1.00 | 22.88 | N |
| ATOM | 6096 | CA | PHE | M | 139 | −11.496 | 4.314 | −12.273 | 1.00 | 22.48 | C |
| ATOM | 6097 | CB | PHE | M | 139 | −12.223 | 4.204 | −13.626 | 1.00 | 21.88 | C |
| ATOM | 6098 | CG | PHE | M | 139 | −12.093 | 5.422 | −14.482 | 1.00 | 19.47 | C |
| ATOM | 6099 | CD1 | PHE | M | 139 | −13.073 | 6.415 | −14.455 | 1.00 | 17.05 | C |
| ATOM | 6100 | CE1 | PHE | M | 139 | −12.957 | 7.565 | −15.244 | 1.00 | 15.63 | C |
| ATOM | 6101 | CZ | PHE | M | 139 | −11.851 | 7.732 | −16.075 | 1.00 | 15.48 | C |
| ATOM | 6102 | CE2 | PHE | M | 139 | −10.857 | 6.745 | −16.117 | 1.00 | 17.65 | C |
| ATOM | 6103 | CD2 | PHE | M | 139 | −10.985 | 5.589 | −15.312 | 1.00 | 18.20 | C |
| ATOM | 6104 | C | PHE | M | 139 | −9.978 | 4.472 | −12.448 | 1.00 | 22.69 | C |
| ATOM | 6105 | O | PHE | M | 139 | −9.408 | 5.522 | −12.147 | 1.00 | 21.96 | O |
| ATOM | 6106 | N | TYR | M | 140 | −9.334 | 3.412 | −12.916 | 1.00 | 22.96 | N |
| ATOM | 6107 | CA | TYR | M | 140 | −7.905 | 3.459 | −13.181 | 1.00 | 23.63 | C |
| ATOM | 6108 | CB | TYR | M | 140 | −7.083 | 3.234 | −11.897 | 1.00 | 23.28 | C |
| ATOM | 6109 | CG | TYR | M | 140 | −5.631 | 3.524 | −12.135 | 1.00 | 23.18 | C |
| ATOM | 6110 | CD1 | TYR | M | 140 | −4.759 | 2.518 | −12.556 | 1.00 | 22.55 | C |
| ATOM | 6111 | CE1 | TYR | M | 140 | −3.414 | 2.789 | −12.815 | 1.00 | 22.75 | C |
| ATOM | 6112 | CZ | TYR | M | 140 | −2.932 | 4.078 | −12.660 | 1.00 | 22.97 | C |
| ATOM | 6113 | OH | TYR | M | 140 | −1.613 | 4.349 | −12.929 | 1.00 | 22.55 | O |
| ATOM | 6114 | CE2 | TYR | M | 140 | −3.779 | 5.098 | −12.251 | 1.00 | 23.44 | C |
| ATOM | 6115 | CD2 | TYR | M | 140 | −5.129 | 4.816 | −11.991 | 1.00 | 22.63 | C |
| ATOM | 6116 | C | TYR | M | 140 | −7.565 | 2.417 | −14.225 | 1.00 | 24.24 | C |
| ATOM | 6117 | O | TYR | M | 140 | −8.045 | 1.282 | −14.130 | 1.00 | 24.47 | O |
| ATOM | 6118 | N | PRO | M | 141 | −6.734 | 2.778 | −15.221 | 1.00 | 25.11 | N |
| ATOM | 6119 | CA | PRO | M | 141 | −6.036 | 4.064 | −15.432 | 1.00 | 25.83 | C |
| ATOM | 6120 | CB | PRO | M | 141 | −4.828 | 3.660 | −16.287 | 1.00 | 25.46 | C |
| ATOM | 6121 | CG | PRO | M | 141 | −5.285 | 2.464 | −17.051 | 1.00 | 25.43 | C |
| ATOM | 6122 | CD | PRO | M | 141 | −6.388 | 1.793 | −16.264 | 1.00 | 24.67 | C |
| ATOM | 6123 | C | PRO | M | 141 | −6.886 | 5.170 | −16.111 | 1.00 | 26.53 | C |
| ATOM | 6124 | O | PRO | M | 141 | −8.102 | 5.006 | −16.246 | 1.00 | 26.20 | O |
| ATOM | 6125 | N | ARG | M | 142 | −6.248 | 6.288 | −16.487 | 1.00 | 27.62 | N |
| ATOM | 6126 | CA | ARG | M | 142 | −6.942 | 7.466 | −17.050 | 1.00 | 28.70 | C |
| ATOM | 6127 | CB | ARG | M | 142 | −5.959 | 8.630 | −17.288 | 1.00 | 28.99 | C |
| ATOM | 6128 | CG | ARG | M | 142 | −6.640 | 10.015 | −17.484 | 1.00 | 31.56 | C |
| ATOM | 6129 | CD | ARG | M | 142 | −5.650 | 11.172 | −17.814 | 1.00 | 35.49 | C |
| ATOM | 6130 | NE | ARG | M | 142 | −6.083 | 12.439 | −17.202 | 1.00 | 37.59 | N |
| ATOM | 6131 | CZ | ARG | M | 142 | −5.520 | 13.008 | −16.125 | 1.00 | 37.63 | C |
| ATOM | 6132 | NH1 | ARG | M | 142 | −4.451 | 12.454 | −15.551 | 1.00 | 36.57 | N |
| ATOM | 6133 | NH2 | ARG | M | 142 | −6.014 | 14.148 | −15.628 | 1.00 | 35.38 | N |
| ATOM | 6134 | C | ARG | M | 142 | −7.686 | 7.120 | −18.343 | 1.00 | 29.03 | C |
| ATOM | 6135 | O | ARG | M | 142 | −8.876 | 7.388 | −18.470 | 1.00 | 28.79 | O |
| ATOM | 6136 | N | GLU | M | 143 | −6.960 | 6.522 | −19.284 | 1.00 | 29.71 | N |
| ATOM | 6137 | CA | GLU | M | 143 | −7.464 | 6.076 | −20.585 | 1.00 | 30.77 | C |
| ATOM | 6138 | CB | GLU | M | 143 | −6.466 | 5.088 | −21.198 | 1.00 | 31.10 | C |
| ATOM | 6139 | CG | GLU | M | 143 | −5.024 | 5.657 | −21.350 | 1.00 | 34.73 | C |
| ATOM | 6140 | CD | GLU | M | 143 | −4.180 | 5.698 | −20.045 | 1.00 | 36.78 | C |
| ATOM | 6141 | OE1 | GLU | M | 143 | −4.661 | 5.287 | −18.972 | 1.00 | 38.82 | O |
| ATOM | 6142 | OE2 | GLU | M | 143 | −3.012 | 6.136 | −20.102 | 1.00 | 36.99 | O |
| ATOM | 6143 | C | GLU | M | 143 | −8.864 | 5.456 | −20.544 | 1.00 | 30.71 | C |
| ATOM | 6144 | O | GLU | M | 143 | −9.099 | 4.451 | −19.873 | 1.00 | 30.18 | O |
| ATOM | 6145 | N | ALA | M | 144 | −9.792 | 6.078 | −21.264 | 1.00 | 30.94 | N |
| ATOM | 6146 | CA | ALA | M | 144 | −11.188 | 5.644 | −21.278 | 1.00 | 31.34 | C |
| ATOM | 6147 | CB | ALA | M | 144 | −11.905 | 6.116 | −20.005 | 1.00 | 30.90 | C |
| ATOM | 6148 | C | ALA | M | 144 | −11.903 | 6.146 | −22.545 | 1.00 | 31.57 | C |
| ATOM | 6149 | O | ALA | M | 144 | −11.432 | 7.069 | −23.195 | 1.00 | 31.92 | O |
| ATOM | 6150 | N | LYS | M | 145 | −13.027 | 5.536 | −22.904 | 1.00 | 31.94 | N |
| ATOM | 6151 | CA | LYS | M | 145 | −13.713 | 5.910 | −24.138 | 1.00 | 32.49 | C |
| ATOM | 6152 | CB | LYS | M | 145 | −13.415 | 4.923 | −25.285 | 1.00 | 32.91 | C |
| ATOM | 6153 | CG | LYS | M | 145 | −13.802 | 5.435 | −26.678 | 1.00 | 35.44 | C |
| ATOM | 6154 | CD | LYS | M | 145 | −14.155 | 4.308 | −27.684 | 1.00 | 39.52 | C |
| ATOM | 6155 | CE | LYS | M | 145 | −12.897 | 3.660 | −28.309 | 1.00 | 42.25 | C |
| ATOM | 6156 | NZ | LYS | M | 145 | −13.192 | 2.568 | −29.294 | 1.00 | 42.89 | N |
| ATOM | 6157 | C | LYS | M | 145 | −15.197 | 5.998 | −23.879 | 1.00 | 32.02 | C |
| ATOM | 6158 | O | LYS | M | 145 | −15.820 | 5.037 | −23.445 | 1.00 | 31.98 | O |
| ATOM | 6159 | N | VAL | M | 146 | −15.746 | 7.178 | −24.124 | 1.00 | 31.77 | N |
| ATOM | 6160 | CA | VAL | M | 146 | −17.174 | 7.401 | −24.027 | 1.00 | 31.48 | C |
| ATOM | 6161 | CB | VAL | M | 146 | −17.500 | 8.695 | −23.230 | 1.00 | 31.55 | C |
| ATOM | 6162 | CG1 | VAL | M | 146 | −18.920 | 9.193 | −23.509 | 1.00 | 30.54 | C |
| ATOM | 6163 | CG2 | VAL | M | 146 | −17.298 | 8.470 | −21.752 | 1.00 | 31.37 | C |
| ATOM | 6164 | C | VAL | M | 146 | −17.693 | 7.513 | −25.445 | 1.00 | 31.42 | C |
| ATOM | 6165 | O | VAL | M | 146 | −17.177 | 8.296 | −26.251 | 1.00 | 31.49 | O |
| ATOM | 6166 | N | GLN | M | 147 | −18.695 | 6.708 | −25.753 | 1.00 | 31.35 | N |
| ATOM | 6167 | CA | GLN | M | 147 | −19.372 | 6.794 | −27.034 | 1.00 | 31.62 | C |
| ATOM | 6168 | CB | GLN | M | 147 | −19.134 | 5.523 | −27.872 | 1.00 | 31.74 | C |
| ATOM | 6169 | CG | GLN | M | 147 | −19.725 | 5.532 | −29.284 | 1.00 | 34.19 | C |
| ATOM | 6170 | CD | GLN | M | 147 | −19.057 | 6.526 | −30.248 | 1.00 | 36.71 | C |
| ATOM | 6171 | OE1 | GLN | M | 147 | −17.886 | 6.374 | −30.623 | 1.00 | 38.85 | O |
| ATOM | 6172 | NE2 | GLN | M | 147 | −19.820 | 7.525 | −30.678 | 1.00 | 37.21 | N |
| ATOM | 6173 | C | GLN | M | 147 | −20.842 | 7.057 | −26.750 | 1.00 | 30.90 | C |
| ATOM | 6174 | O | GLN | M | 147 | −21.505 | 6.279 | −26.080 | 1.00 | 30.44 | O |
| ATOM | 6175 | N | TRP | M | 148 | −21.317 | 8.203 | −27.223 | 1.00 | 31.08 | N |
| ATOM | 6176 | CA | TRP | M | 148 | −22.721 | 8.573 | −27.124 | 1.00 | 31.00 | C |
| ATOM | 6177 | CB | TRP | M | 148 | −22.868 | 10.088 | −27.119 | 1.00 | 30.37 | C |
| ATOM | 6178 | CG | TRP | M | 148 | −22.569 | 10.711 | −25.792 | 1.00 | 28.57 | C |
| ATOM | 6179 | CD1 | TRP | M | 148 | −21.415 | 11.352 | −25.427 | 1.00 | 27.19 | C |
| ATOM | 6180 | NE1 | TRP | M | 148 | −21.508 | 11.792 | −24.129 | 1.00 | 26.25 | N |
| ATOM | 6181 | CE2 | TRP | M | 148 | −22.736 | 11.445 | −23.627 | 1.00 | 26.23 | C |
| ATOM | 6182 | CD2 | TRP | M | 148 | −23.432 | 10.756 | −24.646 | 1.00 | 26.49 | C |
| ATOM | 6183 | CE3 | TRP | M | 148 | −24.723 | 10.279 | −24.379 | 1.00 | 24.80 | C |
| ATOM | 6184 | CZ3 | TRP | M | 148 | −25.272 | 10.504 | −23.122 | 1.00 | 23.42 | C |
| ATOM | 6185 | CH2 | TRP | M | 148 | −24.554 | 11.187 | −22.126 | 1.00 | 23.70 | C |
| ATOM | 6186 | CZ2 | TRP | M | 148 | −23.294 | 11.675 | −22.361 | 1.00 | 24.85 | C |
| ATOM | 6187 | C | TRP | M | 148 | −23.484 | 7.973 | −28.292 | 1.00 | 31.79 | C |
| ATOM | 6188 | O | TRP | M | 148 | −22.962 | 7.881 | −29.405 | 1.00 | 31.65 | O |
| ATOM | 6189 | N | LYS | M | 149 | −24.708 | 7.529 | −28.019 | 1.00 | 32.82 | N |
| ATOM | 6190 | CA | LYS | M | 149 | −25.574 | 6.950 | −29.045 | 1.00 | 33.65 | C |
| ATOM | 6191 | CB | LYS | M | 149 | −25.575 | 5.424 | −29.004 | 1.00 | 33.81 | C |
| ATOM | 6192 | CG | LYS | M | 149 | −24.292 | 4.759 | −29.448 | 1.00 | 34.63 | C |
| ATOM | 6193 | CD | LYS | M | 149 | −24.473 | 3.243 | −29.462 | 1.00 | 36.20 | C |
| ATOM | 6194 | CE | LYS | M | 149 | −23.206 | 2.539 | −29.003 | 1.00 | 37.34 | C |
| ATOM | 6195 | NZ | LYS | M | 149 | −23.016 | 1.258 | −29.737 | 1.00 | 39.14 | N |
| ATOM | 6196 | C | LYS | M | 149 | −26.987 | 7.422 | −28.851 | 1.00 | 34.11 | C |
| ATOM | 6197 | O | LYS | M | 149 | −27.527 | 7.358 | −27.747 | 1.00 | 34.06 | O |
| ATOM | 6198 | N | VAL | M | 150 | −27.580 | 7.886 | −29.942 | 1.00 | 35.08 | N |
| ATOM | 6199 | CA | VAL | M | 150 | −28.973 | 8.292 | −29.963 | 1.00 | 36.08 | C |
| ATOM | 6200 | CB | VAL | M | 150 | −29.115 | 9.763 | −30.401 | 1.00 | 36.00 | C |
| ATOM | 6201 | CG1 | VAL | M | 150 | −30.560 | 10.174 | −30.410 | 1.00 | 36.04 | C |
| ATOM | 6202 | CG2 | VAL | M | 150 | −28.336 | 10.669 | −29.451 | 1.00 | 36.49 | C |
| ATOM | 6203 | C | VAL | M | 150 | −29.708 | 7.330 | −30.897 | 1.00 | 36.71 | C |
| ATOM | 6204 | O | VAL | M | 150 | −29.331 | 7.170 | −32.068 | 1.00 | 36.85 | O |
| ATOM | 6205 | N | ASP | M | 151 | −30.734 | 6.674 | −30.355 | 1.00 | 37.38 | N |
| ATOM | 6206 | CA | ASP | M | 151 | −31.393 | 5.534 | −31.008 | 1.00 | 38.42 | C |
| ATOM | 6207 | CB | ASP | M | 151 | −32.544 | 5.993 | −31.919 | 1.00 | 38.39 | C |
| ATOM | 6208 | CG | ASP | M | 151 | −33.707 | 6.608 | −31.137 | 1.00 | 39.62 | C |
| ATOM | 6209 | OD1 | ASP | M | 151 | −34.177 | 6.008 | −30.136 | 1.00 | 39.08 | O |
| ATOM | 6210 | OD2 | ASP | M | 151 | −34.154 | 7.704 | −31.535 | 1.00 | 41.23 | O |
| ATOM | 6211 | C | ASP | M | 151 | −30.408 | 4.619 | −31.760 | 1.00 | 38.81 | C |
| ATOM | 6212 | O | ASP | M | 151 | −30.621 | 4.287 | −32.931 | 1.00 | 39.24 | O |
| ATOM | 6213 | N | ASN | M | 152 | −29.329 | 4.237 | −31.071 | 1.00 | 39.01 | N |
| ATOM | 6214 | CA | ASN | M | 152 | −28.295 | 3.324 | −31.586 | 1.00 | 39.17 | C |
| ATOM | 6215 | CB | ASN | M | 152 | −28.906 | 2.000 | −32.100 | 1.00 | 39.64 | C |
| ATOM | 6216 | CG | ASN | M | 152 | −27.876 | 0.858 | −32.186 | 1.00 | 41.20 | C |
| ATOM | 6217 | OD1 | ASN | M | 152 | −27.128 | 0.594 | −31.240 | 1.00 | 42.52 | O |
| ATOM | 6218 | ND2 | ASN | M | 152 | −27.842 | 0.180 | −33.330 | 1.00 | 43.09 | N |
| ATOM | 6219 | C | ASN | M | 152 | −27.316 | 3.938 | −32.603 | 1.00 | 38.94 | C |
| ATOM | 6220 | O | ASN | M | 152 | −26.317 | 3.301 | −32.976 | 1.00 | 38.62 | O |
| ATOM | 6221 | N | ALA | M | 153 | −27.580 | 5.177 | −33.028 | 1.00 | 38.67 | N |
| ATOM | 6222 | CA | ALA | M | 153 | −26.670 | 5.879 | −33.942 | 1.00 | 38.32 | C |
| ATOM | 6223 | CB | ALA | M | 153 | −27.442 | 6.844 | −34.845 | 1.00 | 38.19 | C |
| ATOM | 6224 | C | ALA | M | 153 | −25.561 | 6.603 | −33.168 | 1.00 | 37.99 | C |
| ATOM | 6225 | O | ALA | M | 153 | −25.837 | 7.351 | −32.236 | 1.00 | 37.94 | O |
| ATOM | 6226 | N | LEU | M | 154 | −24.314 | 6.364 | −33.565 | 1.00 | 37.72 | N |
| ATOM | 6227 | CA | LEU | M | 154 | −23.134 | 6.873 | −32.860 | 1.00 | 37.67 | C |
| ATOM | 6228 | CB | LEU | M | 154 | −21.874 | 6.119 | −33.304 | 1.00 | 37.91 | C |
| ATOM | 6229 | CG | LEU | M | 154 | −21.671 | 4.649 | −32.911 | 1.00 | 38.76 | C |
| ATOM | 6230 | CD1 | LEU | M | 154 | −22.602 | 3.685 | −33.660 | 1.00 | 39.65 | C |
| ATOM | 6231 | CD2 | LEU | M | 154 | −20.219 | 4.263 | −33.154 | 1.00 | 39.48 | C |
| ATOM | 6232 | C | LEU | M | 154 | −22.928 | 8.360 | −33.096 | 1.00 | 37.32 | C |
| ATOM | 6233 | O | LEU | M | 154 | −22.835 | 8.803 | −34.240 | 1.00 | 37.34 | O |
| ATOM | 6234 | N | GLN | M | 155 | −22.854 | 9.130 | −32.013 | 1.00 | 36.75 | N |
| ATOM | 6235 | CA | GLN | M | 155 | −22.633 | 10.575 | −32.111 | 1.00 | 36.02 | C |
| ATOM | 6236 | CB | GLN | M | 155 | −23.148 | 11.288 | −30.856 | 1.00 | 35.95 | C |
| ATOM | 6237 | CG | GLN | M | 155 | −24.611 | 11.025 | −30.520 | 1.00 | 35.92 | C |
| ATOM | 6238 | CD | GLN | M | 155 | −25.549 | 11.463 | −31.627 | 1.00 | 37.09 | C |
| ATOM | 6239 | OE1 | GLN | M | 155 | −25.746 | 12.660 | −31.857 | 1.00 | 38.08 | O |
| ATOM | 6240 | NE2 | GLN | M | 155 | −26.128 | 10.491 | −32.331 | 1.00 | 36.38 | N |
| ATOM | 6241 | C | GLN | M | 155 | −21.161 | 10.900 | −32.312 | 1.00 | 35.62 | C |
| ATOM | 6242 | O | GLN | M | 155 | −20.287 | 10.127 | −31.911 | 1.00 | 35.36 | O |
| ATOM | 6243 | N | SER | M | 156 | −20.886 | 12.035 | −32.948 | 1.00 | 35.34 | N |
| ATOM | 6244 | CA | SER | M | 156 | −19.543 | 12.635 | −32.880 | 1.00 | 35.41 | C |
| ATOM | 6245 | CB | SER | M | 156 | −18.501 | 11.884 | −33.740 | 1.00 | 35.64 | C |
| ATOM | 6246 | OG | SER | M | 156 | −18.533 | 12.266 | −35.103 | 1.00 | 35.42 | O |
| ATOM | 6247 | C | SER | M | 156 | −19.550 | 14.143 | −33.160 | 1.00 | 34.99 | C |
| ATOM | 6248 | O | SER | M | 156 | −20.263 | 14.616 | −34.051 | 1.00 | 34.97 | O |
| ATOM | 6249 | N | GLY | M | 157 | −18.761 | 14.874 | −32.370 | 1.00 | 34.38 | N |
| ATOM | 6250 | CA | GLY | M | 157 | −18.644 | 16.329 | −32.463 | 1.00 | 33.66 | C |
| ATOM | 6251 | C | GLY | M | 157 | −19.481 | 17.087 | −31.443 | 1.00 | 33.31 | C |
| ATOM | 6252 | O | GLY | M | 157 | −19.107 | 18.191 | −31.032 | 1.00 | 33.27 | O |
| ATOM | 6253 | N | ASN | M | 158 | −20.598 | 16.485 | −31.023 | 1.00 | 32.56 | N |
| ATOM | 6254 | CA | ASN | M | 158 | −21.598 | 17.146 | −30.165 | 1.00 | 32.10 | C |
| ATOM | 6255 | CB | ASN | M | 158 | −23.012 | 16.865 | −30.687 | 1.00 | 32.64 | C |
| ATOM | 6256 | CG | ASN | M | 158 | −23.247 | 15.384 | −30.980 | 1.00 | 33.50 | C |
| ATOM | 6257 | OD1 | ASN | M | 158 | −22.307 | 14.581 | −31.031 | 1.00 | 35.97 | O |
| ATOM | 6258 | ND2 | ASN | M | 158 | −24.503 | 15.023 | −31.183 | 1.00 | 34.40 | N |
| ATOM | 6259 | C | ASN | M | 158 | −21.531 | 16.792 | −28.677 | 1.00 | 31.38 | C |
| ATOM | 6260 | O | ASN | M | 158 | −22.516 | 16.942 | −27.944 | 1.00 | 31.49 | O |
| ATOM | 6261 | N | SER | M | 159 | −20.370 | 16.333 | −28.229 | 1.00 | 30.44 | N |
| ATOM | 6262 | CA | SER | M | 159 | −20.153 | 16.069 | −26.811 | 1.00 | 29.76 | C |
| ATOM | 6263 | CB | SER | M | 159 | −20.218 | 14.559 | −26.501 | 1.00 | 29.54 | C |
| ATOM | 6264 | OG | SER | M | 159 | −19.153 | 13.853 | −27.112 | 1.00 | 28.73 | O |
| ATOM | 6265 | C | SER | M | 159 | −18.827 | 16.655 | −26.362 | 1.00 | 29.24 | C |
| ATOM | 6266 | O | SER | M | 159 | −17.902 | 16.809 | −27.165 | 1.00 | 29.18 | O |
| ATOM | 6267 | N | GLN | M | 160 | −18.745 | 16.998 | −25.081 | 1.00 | 28.92 | N |
| ATOM | 6268 | CA | GLN | M | 160 | −17.469 | 17.412 | −24.484 | 1.00 | 28.30 | C |
| ATOM | 6269 | CB | GLN | M | 160 | −17.473 | 18.903 | −24.175 | 1.00 | 28.03 | C |
| ATOM | 6270 | CG | GLN | M | 160 | −17.225 | 19.785 | −25.379 | 1.00 | 26.05 | C |
| ATOM | 6271 | CD | GLN | M | 160 | −17.355 | 21.241 | −25.028 | 1.00 | 23.74 | C |
| ATOM | 6272 | OE1 | GLN | M | 160 | −18.418 | 21.695 | −24.603 | 1.00 | 22.33 | O |
| ATOM | 6273 | NE2 | GLN | M | 160 | −16.269 | 21.985 | −25.182 | 1.00 | 21.20 | N |
| ATOM | 6274 | C | GLN | M | 160 | −17.149 | 16.627 | −23.228 | 1.00 | 28.14 | C |
| ATOM | 6275 | O | GLN | M | 160 | −18.047 | 16.172 | −22.527 | 1.00 | 27.90 | O |
| ATOM | 6276 | N | GLU | M | 161 | −15.857 | 16.503 | −22.947 | 1.00 | 28.48 | N |
| ATOM | 6277 | CA | GLU | M | 161 | −15.351 | 15.697 | −21.824 | 1.00 | 28.49 | C |
| ATOM | 6278 | CB | GLU | M | 161 | −14.533 | 14.515 | −22.347 | 1.00 | 28.46 | C |
| ATOM | 6279 | CG | GLU | M | 161 | −15.300 | 13.251 | −22.546 | 1.00 | 29.31 | C |
| ATOM | 6280 | CD | GLU | M | 161 | −14.414 | 12.125 | −23.032 | 1.00 | 28.92 | C |
| ATOM | 6281 | OE1 | GLU | M | 161 | −14.979 | 11.093 | −23.435 | 1.00 | 29.89 | O |
| ATOM | 6282 | OE2 | GLU | M | 161 | −13.168 | 12.269 | −23.013 | 1.00 | 27.39 | O |
| ATOM | 6283 | C | GLU | M | 161 | −14.441 | 16.468 | −20.888 | 1.00 | 27.82 | C |
| ATOM | 6284 | O | GLU | M | 161 | −13.609 | 17.265 | −21.324 | 1.00 | 27.75 | O |
| ATOM | 6285 | N | SER | M | 162 | −14.571 | 16.154 | −19.607 | 1.00 | 27.40 | N |
| ATOM | 6286 | CA | SER | M | 162 | −13.690 | 16.654 | −18.562 | 1.00 | 27.04 | C |
| ATOM | 6287 | CB | SER | M | 162 | −14.428 | 17.722 | −17.770 | 1.00 | 26.98 | C |
| ATOM | 6288 | OG | SER | M | 162 | −13.516 | 18.441 | −16.980 | 1.00 | 29.04 | O |
| ATOM | 6289 | C | SER | M | 162 | −13.303 | 15.499 | −17.630 | 1.00 | 26.46 | C |
| ATOM | 6290 | O | SER | M | 162 | −14.147 | 14.655 | −17.319 | 1.00 | 26.40 | O |
| ATOM | 6291 | N | VAL | M | 163 | −12.046 | 15.468 | −17.177 | 1.00 | 26.08 | N |
| ATOM | 6292 | CA | VAL | M | 163 | −11.560 | 14.439 | −16.220 | 1.00 | 25.90 | C |
| ATOM | 6293 | CB | VAL | M | 163 | −10.630 | 13.371 | −16.919 | 1.00 | 26.03 | C |
| ATOM | 6294 | CG1 | VAL | M | 163 | −9.591 | 14.031 | −17.820 | 1.00 | 26.97 | C |
| ATOM | 6295 | CG2 | VAL | M | 163 | −9.943 | 12.461 | −15.907 | 1.00 | 25.87 | C |
| ATOM | 6296 | C | VAL | M | 163 | −10.879 | 15.047 | −14.979 | 1.00 | 25.61 | C |
| ATOM | 6297 | O | VAL | M | 163 | −10.060 | 15.951 | −15.112 | 1.00 | 25.68 | O |
| ATOM | 6298 | N | THR | M | 164 | −11.222 | 14.571 | −13.777 | 1.00 | 25.22 | N |
| ATOM | 6299 | CA | THR | M | 164 | −10.548 | 15.051 | −12.555 | 1.00 | 24.61 | C |
| ATOM | 6300 | CB | THR | M | 164 | −11.186 | 14.531 | −11.249 | 1.00 | 24.45 | C |
| ATOM | 6301 | OG1 | THR | M | 164 | −11.295 | 13.103 | −11.300 | 1.00 | 24.19 | O |
| ATOM | 6302 | CG2 | THR | M | 164 | −12.543 | 15.173 | −10.988 | 1.00 | 23.43 | C |
| ATOM | 6303 | C | THR | M | 164 | −9.090 | 14.633 | −12.507 | 1.00 | 24.81 | C |
| ATOM | 6304 | O | THR | M | 164 | −8.671 | 13.716 | −13.199 | 1.00 | 24.90 | O |
| ATOM | 6305 | N | GLU | M | 165 | −8.320 | 15.318 | −11.678 | 1.00 | 25.11 | N |
| ATOM | 6306 | CA | GLU | M | 165 | −6.968 | 14.905 | −11.380 | 1.00 | 25.62 | C |
| ATOM | 6307 | CB | GLU | M | 165 | −6.183 | 16.089 | −10.793 | 1.00 | 25.80 | C |
| ATOM | 6308 | CG | GLU | M | 165 | −5.863 | 17.189 | −11.818 | 1.00 | 26.99 | C |
| ATOM | 6309 | CD | GLU | M | 165 | −5.144 | 16.654 | −13.066 | 1.00 | 30.43 | C |
| ATOM | 6310 | OE1 | GLU | M | 165 | −4.054 | 16.054 | −12.910 | 1.00 | 31.74 | O |
| ATOM | 6311 | OE2 | GLU | M | 165 | −5.671 | 16.818 | −14.198 | 1.00 | 30.03 | O |
| ATOM | 6312 | C | GLU | M | 165 | −7.015 | 13.705 | −10.419 | 1.00 | 25.70 | C |
| ATOM | 6313 | O | GLU | M | 165 | −8.042 | 13.454 | −9.773 | 1.00 | 25.18 | O |
| ATOM | 6314 | N | GLN | M | 166 | −5.911 | 12.965 | −10.340 | 1.00 | 26.04 | N |
| ATOM | 6315 | CA | GLN | M | 166 | −5.826 | 11.772 | −9.497 | 1.00 | 26.71 | C |
| ATOM | 6316 | CB | GLN | M | 166 | −4.423 | 11.192 | −9.552 | 1.00 | 26.56 | C |
| ATOM | 6317 | CG | GLN | M | 166 | −4.347 | 9.777 | −9.018 | 1.00 | 27.48 | C |
| ATOM | 6318 | CD | GLN | M | 166 | −3.038 | 9.098 | −9.327 | 1.00 | 28.19 | C |
| ATOM | 6319 | OE1 | GLN | M | 166 | −1.976 | 9.719 | −9.293 | 1.00 | 30.93 | O |
| ATOM | 6320 | NE2 | GLN | M | 166 | −3.102 | 7.811 | −9.617 | 1.00 | 28.53 | N |
| ATOM | 6321 | C | GLN | M | 166 | −6.242 | 11.974 | −8.029 | 1.00 | 27.12 | C |
| ATOM | 6322 | O | GLN | M | 166 | −5.726 | 12.851 | −7.340 | 1.00 | 27.14 | O |
| ATOM | 6323 | N | ASP | M | 167 | −7.179 | 11.157 | −7.559 | 1.00 | 27.66 | N |
| ATOM | 6324 | CA | ASP | M | 167 | −7.600 | 11.223 | −6.163 | 1.00 | 28.50 | C |
| ATOM | 6325 | CB | ASP | M | 167 | −8.769 | 10.283 | −5.889 | 1.00 | 28.11 | C |
| ATOM | 6326 | CG | ASP | M | 167 | −9.339 | 10.468 | −4.514 | 1.00 | 29.80 | C |
| ATOM | 6327 | OD1 | ASP | M | 167 | −9.015 | 9.664 | −3.619 | 1.00 | 32.31 | O |
| ATOM | 6328 | OD2 | ASP | M | 167 | −10.114 | 11.425 | −4.309 | 1.00 | 32.30 | O |
| ATOM | 6329 | C | ASP | M | 167 | −6.427 | 10.901 | −5.235 | 1.00 | 28.66 | C |
| ATOM | 6330 | O | ASP | M | 167 | −5.617 | 10.015 | −5.521 | 1.00 | 29.09 | O |
| ATOM | 6331 | N | SER | M | 168 | −6.334 | 11.634 | −4.134 | 1.00 | 29.03 | N |
| ATOM | 6332 | CA | SER | M | 168 | −5.196 | 11.509 | −3.225 | 1.00 | 29.29 | C |
| ATOM | 6333 | CB | SER | M | 168 | −4.964 | 12.814 | −2.442 | 1.00 | 29.31 | C |
| ATOM | 6334 | OG | SER | M | 168 | −6.197 | 13.420 | −2.089 | 1.00 | 29.51 | O |
| ATOM | 6335 | C | SER | M | 168 | −5.361 | 10.319 | −2.291 | 1.00 | 29.18 | C |
| ATOM | 6336 | O | SER | M | 168 | −4.372 | 9.782 | −1.793 | 1.00 | 29.38 | O |
| ATOM | 6337 | N | LYS | M | 169 | −6.607 | 9.906 | −2.075 | 1.00 | 28.99 | N |
| ATOM | 6338 | CA | LYS | M | 169 | −6.901 | 8.739 | −1.250 | 1.00 | 29.17 | C |
| ATOM | 6339 | CB | LYS | M | 169 | −8.265 | 8.871 | −0.547 | 1.00 | 29.67 | C |
| ATOM | 6340 | CG | LYS | M | 169 | −8.308 | 10.000 | 0.501 | 1.00 | 32.72 | C |
| ATOM | 6341 | CD | LYS | M | 169 | −9.554 | 9.956 | 1.413 | 1.00 | 37.04 | C |
| ATOM | 6342 | CE | LYS | M | 169 | −9.358 | 10.870 | 2.656 | 1.00 | 38.32 | C |
| ATOM | 6343 | NZ | LYS | M | 169 | −10.539 | 10.949 | 3.580 | 1.00 | 37.42 | N |
| ATOM | 6344 | C | LYS | M | 169 | −6.808 | 7.422 | −2.025 | 1.00 | 28.51 | C |
| ATOM | 6345 | O | LYS | M | 169 | −6.080 | 6.518 | −1.605 | 1.00 | 28.73 | O |
| ATOM | 6346 | N | ASP | M | 170 | −7.535 | 7.302 | −3.142 | 1.00 | 27.47 | N |
| ATOM | 6347 | CA | ASP | M | 170 | −7.620 | 6.013 | −3.860 | 1.00 | 26.09 | C |
| ATOM | 6348 | CB | ASP | M | 170 | −9.077 | 5.572 | −4.071 | 1.00 | 26.40 | C |
| ATOM | 6349 | CG | ASP | M | 170 | −9.882 | 6.534 | −4.962 | 1.00 | 26.82 | C |
| ATOM | 6350 | OD1 | ASP | M | 170 | −9.309 | 7.162 | −5.876 | 1.00 | 26.64 | O |
| ATOM | 6351 | OD2 | ASP | M | 170 | −11.112 | 6.647 | −4.755 | 1.00 | 26.77 | O |
| ATOM | 6352 | C | ASP | M | 170 | −6.868 | 5.951 | −5.178 | 1.00 | 25.26 | C |
| ATOM | 6353 | O | ASP | M | 170 | −6.896 | 4.925 | −5.846 | 1.00 | 25.46 | O |
| ATOM | 6354 | N | SER | M | 171 | −6.219 | 7.050 | −5.557 | 1.00 | 23.85 | N |
| ATOM | 6355 | CA | SER | M | 171 | −5.464 | 7.120 | −6.798 | 1.00 | 23.05 | C |
| ATOM | 6356 | CB | SER | M | 171 | −4.275 | 6.159 | −6.759 | 1.00 | 23.02 | C |
| ATOM | 6357 | OG | SER | M | 171 | −3.548 | 6.323 | −5.560 | 1.00 | 24.13 | O |
| ATOM | 6358 | C | SER | M | 171 | −6.274 | 6.911 | −8.086 | 1.00 | 22.20 | C |
| ATOM | 6359 | O | SER | M | 171 | −5.709 | 6.539 | −9.120 | 1.00 | 21.66 | O |
| ATOM | 6360 | N | THR | M | 172 | −7.584 | 7.141 | −8.033 | 1.00 | 21.60 | N |
| ATOM | 6361 | CA | THR | M | 172 | −8.414 | 7.018 | −9.238 | 1.00 | 21.25 | C |
| ATOM | 6362 | CB | THR | M | 172 | −9.737 | 6.252 | −8.979 | 1.00 | 21.37 | C |
| ATOM | 6363 | OG1 | THR | M | 172 | −10.558 | 6.972 | −8.052 | 1.00 | 19.88 | O |
| ATOM | 6364 | CG2 | THR | M | 172 | −9.468 | 4.826 | −8.450 | 1.00 | 21.62 | C |
| ATOM | 6365 | C | THR | M | 172 | −8.723 | 8.359 | −9.922 | 1.00 | 21.61 | C |
| ATOM | 6366 | O | THR | M | 172 | −8.365 | 9.451 | −9.414 | 1.00 | 22.12 | O |
| ATOM | 6367 | N | TYR | M | 173 | −9.378 | 8.257 | −11.078 | 1.00 | 21.02 | N |
| ATOM | 6368 | CA | TYR | M | 173 | −9.864 | 9.391 | −11.833 | 1.00 | 20.41 | C |
| ATOM | 6369 | CB | TYR | M | 173 | −9.277 | 9.394 | −13.243 | 1.00 | 20.46 | C |
| ATOM | 6370 | CG | TYR | M | 173 | −7.780 | 9.399 | −13.283 | 1.00 | 19.69 | C |
| ATOM | 6371 | CD1 | TYR | M | 173 | −7.068 | 8.201 | −13.340 | 1.00 | 19.53 | C |
| ATOM | 6372 | CE1 | TYR | M | 173 | −5.673 | 8.199 | −13.352 | 1.00 | 20.47 | C |
| ATOM | 6373 | CZ | TYR | M | 173 | −4.990 | 9.411 | −13.319 | 1.00 | 20.30 | C |
| ATOM | 6374 | OH | TYR | M | 173 | −3.616 | 9.409 | −13.359 | 1.00 | 21.34 | O |
| ATOM | 6375 | CE2 | TYR | M | 173 | −5.684 | 10.616 | −13.268 | 1.00 | 18.93 | C |
| ATOM | 6376 | CD2 | TYR | M | 173 | −7.069 | 10.601 | −13.249 | 1.00 | 18.98 | C |
| ATOM | 6377 | C | TYR | M | 173 | −11.363 | 9.290 | −11.953 | 1.00 | 20.60 | C |
| ATOM | 6378 | O | TYR | M | 173 | −11.944 | 8.225 | −11.743 | 1.00 | 20.92 | O |
| ATOM | 6379 | N | SER | M | 174 | −11.980 | 10.416 | −12.289 | 1.00 | 20.56 | N |
| ATOM | 6380 | CA | SER | M | 174 | −13.352 | 10.460 | −12.729 | 1.00 | 20.89 | C |
| ATOM | 6381 | CB | SER | M | 174 | −14.233 | 10.979 | −11.610 | 1.00 | 20.75 | C |
| ATOM | 6382 | OG | SER | M | 174 | −14.229 | 10.060 | −10.539 | 1.00 | 20.80 | O |
| ATOM | 6383 | C | SER | M | 174 | −13.485 | 11.317 | −14.005 | 1.00 | 21.41 | C |
| ATOM | 6384 | O | SER | M | 174 | −12.592 | 12.101 | −14.342 | 1.00 | 21.12 | O |
| ATOM | 6385 | N | LEU | M | 175 | −14.598 | 11.156 | −14.714 | 1.00 | 21.74 | N |
| ATOM | 6386 | CA | LEU | M | 175 | −14.794 | 11.820 | −15.993 | 1.00 | 22.37 | C |
| ATOM | 6387 | CB | LEU | M | 175 | −14.318 | 10.921 | −17.143 | 1.00 | 22.44 | C |
| ATOM | 6388 | CG | LEU | M | 175 | −14.372 | 11.429 | −18.600 | 1.00 | 22.80 | C |
| ATOM | 6389 | CD1 | LEU | M | 175 | −13.290 | 10.795 | −19.460 | 1.00 | 22.47 | C |
| ATOM | 6390 | CD2 | LEU | M | 175 | −15.730 | 11.225 | −19.239 | 1.00 | 21.69 | C |
| ATOM | 6391 | C | LEU | M | 175 | −16.257 | 12.099 | −16.167 | 1.00 | 22.99 | C |
| ATOM | 6392 | O | LEU | M | 175 | −17.075 | 11.214 | −15.905 | 1.00 | 23.18 | O |
| ATOM | 6393 | N | SER | M | 176 | −16.596 | 13.315 | −16.605 | 1.00 | 23.78 | N |
| ATOM | 6394 | CA | SER | M | 176 | −17.950 | 13.572 | −17.100 | 1.00 | 24.66 | C |
| ATOM | 6395 | CB | SER | M | 176 | −18.748 | 14.527 | −16.219 | 1.00 | 24.41 | C |
| ATOM | 6396 | OG | SER | M | 176 | −18.277 | 15.845 | −16.321 | 1.00 | 26.23 | O |
| ATOM | 6397 | C | SER | M | 176 | −17.969 | 14.009 | −18.558 | 1.00 | 25.48 | C |
| ATOM | 6398 | O | SER | M | 176 | −17.118 | 14.798 | −19.008 | 1.00 | 25.19 | O |
| ATOM | 6399 | N | SER | M | 177 | −18.939 | 13.440 | −19.281 | 1.00 | 26.12 | N |
| ATOM | 6400 | CA | SER | M | 177 | −19.203 | 13.736 | −20.683 | 1.00 | 26.25 | C |
| ATOM | 6401 | CB | SER | M | 177 | −19.146 | 12.457 | −21.515 | 1.00 | 26.33 | C |
| ATOM | 6402 | OG | SER | M | 177 | −19.010 | 12.733 | −22.903 | 1.00 | 27.02 | O |
| ATOM | 6403 | C | SER | M | 177 | −20.589 | 14.352 | −20.776 | 1.00 | 26.53 | C |
| ATOM | 6404 | O | SER | M | 177 | −21.539 | 13.876 | −20.148 | 1.00 | 26.01 | O |
| ATOM | 6405 | N | THR | M | 178 | −20.685 | 15.440 | −21.530 | 1.00 | 27.04 | N |
| ATOM | 6406 | CA | THR | M | 178 | −21.952 | 16.119 | −21.721 | 1.00 | 28.07 | C |
| ATOM | 6407 | CB | THR | M | 178 | −21.940 | 17.560 | −21.120 | 1.00 | 28.15 | C |
| ATOM | 6408 | OG1 | THR | M | 178 | −21.469 | 17.513 | −19.767 | 1.00 | 28.54 | O |
| ATOM | 6409 | CG2 | THR | M | 178 | −23.331 | 18.154 | −21.115 | 1.00 | 27.67 | C |
| ATOM | 6410 | C | THR | M | 178 | −22.268 | 16.137 | −23.213 | 1.00 | 28.58 | C |
| ATOM | 6411 | O | THR | M | 178 | −21.478 | 16.635 | −24.023 | 1.00 | 28.43 | O |
| ATOM | 6412 | N | LEU | M | 179 | −23.399 | 15.539 | −23.572 | 1.00 | 29.30 | N |
| ATOM | 6413 | CA | LEU | M | 179 | −23.917 | 15.635 | −24.931 | 1.00 | 30.55 | C |
| ATOM | 6414 | CB | LEU | M | 179 | −24.609 | 14.337 | −25.367 | 1.00 | 30.11 | C |
| ATOM | 6415 | CG | LEU | M | 179 | −25.144 | 14.271 | −26.807 | 1.00 | 30.08 | C |
| ATOM | 6416 | CD1 | LEU | M | 179 | −24.055 | 13.905 | −27.834 | 1.00 | 29.06 | C |
| ATOM | 6417 | CD2 | LEU | M | 179 | −26.302 | 13.289 | −26.885 | 1.00 | 29.97 | C |
| ATOM | 6418 | C | LEU | M | 179 | −24.893 | 16.800 | −24.962 | 1.00 | 31.44 | C |
| ATOM | 6419 | O | LEU | M | 179 | −25.772 | 16.904 | −24.104 | 1.00 | 31.32 | O |
| ATOM | 6420 | N | THR | M | 180 | −24.714 | 17.698 | −25.925 | 1.00 | 32.90 | N |
| ATOM | 6421 | CA | THR | M | 180 | −25.608 | 18.852 | −26.022 | 1.00 | 34.52 | C |
| ATOM | 6422 | CB | THR | M | 180 | −24.880 | 20.204 | −25.814 | 1.00 | 34.41 | C |
| ATOM | 6423 | OG1 | THR | M | 180 | −24.077 | 20.142 | −24.627 | 1.00 | 34.25 | O |
| ATOM | 6424 | CG2 | THR | M | 180 | −25.895 | 21.329 | −25.646 | 1.00 | 35.30 | C |
| ATOM | 6425 | C | THR | M | 180 | −26.406 | 18.829 | −27.319 | 1.00 | 35.41 | C |
| ATOM | 6426 | O | THR | M | 180 | −25.864 | 18.576 | −28.398 | 1.00 | 35.62 | O |
| ATOM | 6427 | N | LEU | M | 181 | −27.708 | 19.059 | −27.182 | 1.00 | 36.72 | N |
| ATOM | 6428 | CA | LEU | M | 181 | −28.630 | 19.083 | −28.304 | 1.00 | 37.94 | C |
| ATOM | 6429 | CB | LEU | M | 181 | −29.388 | 17.754 | −28.404 | 1.00 | 37.78 | C |
| ATOM | 6430 | CG | LEU | M | 181 | −28.616 | 16.464 | −28.667 | 1.00 | 37.80 | C |
| ATOM | 6431 | CD1 | LEU | M | 181 | −29.562 | 15.284 | −28.567 | 1.00 | 38.29 | C |
| ATOM | 6432 | CD2 | LEU | M | 181 | −27.904 | 16.483 | −30.032 | 1.00 | 37.91 | C |
| ATOM | 6433 | C | LEU | M | 181 | −29.632 | 20.207 | −28.119 | 1.00 | 38.96 | C |
| ATOM | 6434 | O | LEU | M | 181 | −29.905 | 20.636 | −26.987 | 1.00 | 38.77 | O |
| ATOM | 6435 | N | SER | M | 182 | −30.178 | 20.681 | −29.236 | 1.00 | 40.30 | N |
| ATOM | 6436 | CA | SER | M | 182 | −31.333 | 21.562 | −29.197 | 1.00 | 41.85 | C |
| ATOM | 6437 | CB | SER | M | 182 | −31.596 | 22.183 | −30.577 | 1.00 | 42.08 | C |
| ATOM | 6438 | OG | SER | M | 182 | −32.048 | 21.215 | −31.516 | 1.00 | 43.49 | O |
| ATOM | 6439 | C | SER | M | 182 | −32.514 | 20.716 | −28.740 | 1.00 | 42.40 | C |
| ATOM | 6440 | O | SER | M | 182 | −32.547 | 19.515 | −28.998 | 1.00 | 42.70 | O |
| ATOM | 6441 | N | LYS | M | 183 | −33.463 | 21.334 | −28.044 | 1.00 | 43.33 | N |
| ATOM | 6442 | CA | LYS | M | 183 | −34.660 | 20.637 | −27.578 | 1.00 | 43.99 | C |
| ATOM | 6443 | CB | LYS | M | 183 | −35.642 | 21.628 | −26.955 | 1.00 | 43.99 | C |
| ATOM | 6444 | CG | LYS | M | 183 | −36.788 | 20.996 | −26.180 | 1.00 | 44.68 | C |
| ATOM | 6445 | CD | LYS | M | 183 | −37.865 | 22.035 | −25.849 | 1.00 | 45.60 | C |
| ATOM | 6446 | CE | LYS | M | 183 | −39.024 | 21.414 | −25.076 | 1.00 | 46.09 | C |
| ATOM | 6447 | NZ | LYS | M | 183 | −40.247 | 22.267 | −25.122 | 1.00 | 46.03 | N |
| ATOM | 6448 | C | LYS | M | 183 | −35.314 | 19.862 | −28.728 | 1.00 | 44.60 | C |
| ATOM | 6449 | O | LYS | M | 183 | −35.565 | 18.662 | −28.604 | 1.00 | 44.79 | O |
| ATOM | 6450 | N | ALA | M | 184 | −35.548 | 20.540 | −29.851 | 1.00 | 45.30 | N |
| ATOM | 6451 | CA | ALA | M | 184 | −36.145 | 19.907 | −31.037 | 1.00 | 46.02 | C |
| ATOM | 6452 | CB | ALA | M | 184 | −36.025 | 20.815 | −32.267 | 1.00 | 45.83 | C |
| ATOM | 6453 | C | ALA | M | 184 | −35.555 | 18.522 | −31.330 | 1.00 | 46.47 | C |
| ATOM | 6454 | O | ALA | M | 184 | −36.292 | 17.536 | −31.380 | 1.00 | 46.87 | O |
| ATOM | 6455 | N | ASP | M | 185 | −34.232 | 18.457 | −31.498 | 1.00 | 46.66 | N |
| ATOM | 6456 | CA | ASP | M | 185 | −33.536 | 17.205 | −31.808 | 1.00 | 46.79 | C |
| ATOM | 6457 | CB | ASP | M | 185 | −32.061 | 17.472 | −32.106 | 1.00 | 47.08 | C |
| ATOM | 6458 | CG | ASP | M | 185 | −31.866 | 18.468 | −33.219 | 1.00 | 47.80 | C |
| ATOM | 6459 | OD1 | ASP | M | 185 | −32.861 | 18.837 | −33.874 | 1.00 | 48.55 | O |
| ATOM | 6460 | OD2 | ASP | M | 185 | −30.712 | 18.886 | −33.442 | 1.00 | 49.51 | O |
| ATOM | 6461 | C | ASP | M | 185 | −33.638 | 16.178 | −30.690 | 1.00 | 46.65 | C |
| ATOM | 6462 | O | ASP | M | 185 | −33.641 | 14.973 | −30.943 | 1.00 | 46.65 | O |
| ATOM | 6463 | N | TYR | M | 186 | −33.708 | 16.664 | −29.456 | 1.00 | 46.51 | N |
| ATOM | 6464 | CA | TYR | M | 186 | −33.785 | 15.799 | −28.294 | 1.00 | 46.27 | C |
| ATOM | 6465 | CB | TYR | M | 186 | −33.487 | 16.593 | −27.011 | 1.00 | 45.89 | C |
| ATOM | 6466 | CG | TYR | M | 186 | −33.783 | 15.859 | −25.717 | 1.00 | 43.78 | C |
| ATOM | 6467 | CD1 | TYR | M | 186 | −32.977 | 14.809 | −25.285 | 1.00 | 41.71 | C |
| ATOM | 6468 | CE1 | TYR | M | 186 | −33.253 | 14.133 | −24.110 | 1.00 | 40.95 | C |
| ATOM | 6469 | CZ | TYR | M | 186 | −34.341 | 14.515 | −23.341 | 1.00 | 40.96 | C |
| ATOM | 6470 | OH | TYR | M | 186 | −34.626 | 13.856 | −22.167 | 1.00 | 39.86 | O |
| ATOM | 6471 | CE2 | TYR | M | 186 | −35.147 | 15.560 | −23.742 | 1.00 | 41.20 | C |
| ATOM | 6472 | CD2 | TYR | M | 186 | −34.865 | 16.224 | −24.928 | 1.00 | 42.15 | C |
| ATOM | 6473 | C | TYR | M | 186 | −35.162 | 15.146 | −28.251 | 1.00 | 46.81 | C |
| ATOM | 6474 | O | TYR | M | 186 | −35.280 | 13.938 | −28.008 | 1.00 | 46.65 | O |
| ATOM | 6475 | N | GLU | M | 187 | −36.192 | 15.949 | −28.515 | 1.00 | 47.26 | N |
| ATOM | 6476 | CA | GLU | M | 187 | −37.583 | 15.479 | −28.505 | 1.00 | 47.83 | C |
| ATOM | 6477 | CB | GLU | M | 187 | −38.555 | 16.651 | −28.307 | 1.00 | 47.88 | C |
| ATOM | 6478 | CG | GLU | M | 187 | −38.424 | 17.352 | −26.963 | 1.00 | 48.97 | C |
| ATOM | 6479 | CD | GLU | M | 187 | −39.763 | 17.781 | −26.380 | 1.00 | 50.62 | C |
| ATOM | 6480 | OE1 | GLU | M | 187 | −40.690 | 18.100 | −27.159 | 1.00 | 51.26 | O |
| ATOM | 6481 | OE2 | GLU | M | 187 | −39.888 | 17.798 | −25.134 | 1.00 | 50.95 | O |
| ATOM | 6482 | C | GLU | M | 187 | −37.937 | 14.698 | −29.772 | 1.00 | 47.76 | C |
| ATOM | 6483 | O | GLU | M | 187 | −39.099 | 14.383 | −30.013 | 1.00 | 47.98 | O |
| ATOM | 6484 | N | LYS | M | 188 | −36.918 | 14.376 | −30.560 | 1.00 | 47.80 | N |
| ATOM | 6485 | CA | LYS | M | 188 | −37.085 | 13.722 | −31.849 | 1.00 | 47.88 | C |
| ATOM | 6486 | CB | LYS | M | 188 | −36.397 | 14.581 | −32.921 | 1.00 | 48.05 | C |
| ATOM | 6487 | CG | LYS | M | 188 | −36.787 | 14.333 | −34.373 | 1.00 | 49.58 | C |
| ATOM | 6488 | CD | LYS | M | 188 | −35.842 | 15.104 | −35.318 | 1.00 | 51.51 | C |
| ATOM | 6489 | CE | LYS | M | 188 | −35.427 | 14.270 | −36.542 | 1.00 | 52.43 | C |
| ATOM | 6490 | NZ | LYS | M | 188 | −36.509 | 14.129 | −37.565 | 1.00 | 52.37 | N |
| ATOM | 6491 | C | LYS | M | 188 | −36.504 | 12.292 | −31.801 | 1.00 | 47.56 | C |
| ATOM | 6492 | O | LYS | M | 188 | −36.501 | 11.577 | −32.807 | 1.00 | 47.68 | O |
| ATOM | 6493 | N | HIS | M | 189 | −36.018 | 11.882 | −30.626 | 1.00 | 46.86 | N |
| ATOM | 6494 | CA | HIS | M | 189 | −35.485 | 10.531 | −30.432 | 1.00 | 45.94 | C |
| ATOM | 6495 | CB | HIS | M | 189 | −33.967 | 10.536 | −30.562 | 1.00 | 46.03 | C |
| ATOM | 6496 | CG | HIS | M | 189 | −33.457 | 11.106 | −31.851 | 1.00 | 46.57 | C |
| ATOM | 6497 | ND1 | HIS | M | 189 | −33.105 | 12.433 | −31.990 | 1.00 | 47.22 | N |
| ATOM | 6498 | CE1 | HIS | M | 189 | −32.665 | 12.647 | −33.218 | 1.00 | 46.71 | C |
| ATOM | 6499 | NE2 | HIS | M | 189 | −32.717 | 11.507 | −33.882 | 1.00 | 46.59 | N |
| ATOM | 6500 | CD2 | HIS | M | 189 | −33.201 | 10.525 | −33.048 | 1.00 | 47.39 | C |
| ATOM | 6501 | C | HIS | M | 189 | −35.895 | 9.957 | −29.067 | 1.00 | 45.39 | C |
| ATOM | 6502 | O | HIS | M | 189 | −36.369 | 10.694 | −28.196 | 1.00 | 44.83 | O |
| ATOM | 6503 | N | LYS | M | 190 | −35.724 | 8.645 | −28.880 | 1.00 | 44.75 | N |
| ATOM | 6504 | CA | LYS | M | 190 | −36.089 | 8.009 | −27.600 | 1.00 | 44.28 | C |
| ATOM | 6505 | CB | LYS | M | 190 | −37.160 | 6.925 | −27.804 | 1.00 | 44.52 | C |
| ATOM | 6506 | CG | LYS | M | 190 | −37.814 | 6.442 | −26.488 | 1.00 | 46.31 | C |
| ATOM | 6507 | CD | LYS | M | 190 | −39.169 | 5.733 | −26.696 | 1.00 | 49.17 | C |
| ATOM | 6508 | CE | LYS | M | 190 | −40.366 | 6.705 | −26.654 | 1.00 | 50.11 | C |
| ATOM | 6509 | NZ | LYS | M | 190 | −41.662 | 6.049 | −27.047 | 1.00 | 50.16 | N |
| ATOM | 6510 | C | LYS | M | 190 | −34.915 | 7.449 | −26.767 | 1.00 | 43.41 | C |
| ATOM | 6511 | O | LYS | M | 190 | −34.873 | 7.631 | −25.552 | 1.00 | 43.04 | O |
| ATOM | 6512 | N | VAL | M | 191 | −33.977 | 6.771 | −27.424 | 1.00 | 42.47 | N |
| ATOM | 6513 | CA | VAL | M | 191 | −32.954 | 5.996 | −26.721 | 1.00 | 41.39 | C |
| ATOM | 6514 | CB | VAL | M | 191 | −32.723 | 4.601 | −27.392 | 1.00 | 41.56 | C |
| ATOM | 6515 | CG1 | VAL | M | 191 | −31.720 | 3.757 | −26.588 | 1.00 | 41.79 | C |
| ATOM | 6516 | CG2 | VAL | M | 191 | −34.039 | 3.842 | −27.530 | 1.00 | 41.61 | C |
| ATOM | 6517 | C | VAL | M | 191 | −31.629 | 6.757 | −26.568 | 1.00 | 40.39 | C |
| ATOM | 6518 | O | VAL | M | 191 | −30.869 | 6.943 | −27.533 | 1.00 | 40.08 | O |
| ATOM | 6519 | N | TYR | M | 192 | −31.358 | 7.194 | −25.343 | 1.00 | 39.06 | N |
| ATOM | 6520 | CA | TYR | M | 192 | −30.090 | 7.857 | −25.056 | 1.00 | 37.64 | C |
| ATOM | 6521 | CB | TYR | M | 192 | −30.307 | 9.205 | −24.377 | 1.00 | 37.38 | C |
| ATOM | 6522 | CG | TYR | M | 192 | −30.999 | 10.154 | −25.308 | 1.00 | 37.43 | C |
| ATOM | 6523 | CD1 | TYR | M | 192 | −32.390 | 10.272 | −25.302 | 1.00 | 37.71 | C |
| ATOM | 6524 | CE1 | TYR | M | 192 | −33.030 | 11.121 | −26.178 | 1.00 | 38.89 | C |
| ATOM | 6525 | CZ | TYR | M | 192 | −32.277 | 11.851 | −27.089 | 1.00 | 39.25 | C |
| ATOM | 6526 | OH | TYR | M | 192 | −32.912 | 12.705 | −27.968 | 1.00 | 41.07 | O |
| ATOM | 6527 | CE2 | TYR | M | 192 | −30.895 | 11.739 | −27.115 | 1.00 | 37.87 | C |
| ATOM | 6528 | CD2 | TYR | M | 192 | −30.271 | 10.891 | −26.242 | 1.00 | 36.63 | C |
| ATOM | 6529 | C | TYR | M | 192 | −29.165 | 6.953 | −24.280 | 1.00 | 36.81 | C |
| ATOM | 6530 | O | TYR | M | 192 | −29.488 | 6.504 | −23.176 | 1.00 | 36.23 | O |
| ATOM | 6531 | N | ALA | M | 193 | −28.019 | 6.681 | −24.896 | 1.00 | 36.00 | N |
| ATOM | 6532 | CA | ALA | M | 193 | −27.079 | 5.706 | −24.380 | 1.00 | 35.38 | C |
| ATOM | 6533 | CB | ALA | M | 193 | −27.064 | 4.469 | −25.266 | 1.00 | 35.35 | C |
| ATOM | 6534 | C | ALA | M | 193 | −25.678 | 6.262 | −24.239 | 1.00 | 34.63 | C |
| ATOM | 6535 | O | ALA | M | 193 | −25.197 | 7.013 | −25.091 | 1.00 | 34.51 | O |
| ATOM | 6536 | N | CYS | M | 194 | −25.037 | 5.866 | −23.147 | 1.00 | 34.19 | N |
| ATOM | 6537 | CA | CYS | M | 194 | −23.633 | 6.142 | −22.900 | 1.00 | 33.69 | C |
| ATOM | 6538 | CB | CYS | M | 194 | −23.469 | 6.859 | −21.553 | 1.00 | 33.57 | C |
| ATOM | 6539 | SG | CYS | M | 194 | −21.763 | 7.293 | −21.129 | 1.00 | 34.55 | S |
| ATOM | 6540 | C | CYS | M | 194 | −22.908 | 4.795 | −22.892 | 1.00 | 33.33 | C |
| ATOM | 6541 | O | CYS | M | 194 | −23.207 | 3.934 | −22.058 | 1.00 | 32.98 | O |
| ATOM | 6542 | N | GLU | M | 195 | −21.981 | 4.615 | −23.835 | 1.00 | 32.81 | N |
| ATOM | 6543 | CA | GLU | M | 195 | −21.209 | 3.378 | −23.959 | 1.00 | 32.44 | C |
| ATOM | 6544 | CB | GLU | M | 195 | −21.276 | 2.813 | −25.380 | 1.00 | 32.55 | C |
| ATOM | 6545 | CG | GLU | M | 195 | −20.651 | 1.433 | −25.510 | 1.00 | 33.59 | C |
| ATOM | 6546 | CD | GLU | M | 195 | −20.348 | 1.053 | −26.946 | 1.00 | 36.39 | C |
| ATOM | 6547 | OE1 | GLU | M | 195 | −19.445 | 1.669 | −27.571 | 1.00 | 37.84 | O |
| ATOM | 6548 | OE2 | GLU | M | 195 | −21.006 | 0.118 | −27.445 | 1.00 | 37.59 | O |
| ATOM | 6549 | C | GLU | M | 195 | −19.753 | 3.597 | −23.573 | 1.00 | 31.73 | C |
| ATOM | 6550 | O | GLU | M | 195 | −19.028 | 4.321 | −24.258 | 1.00 | 31.50 | O |
| ATOM | 6551 | N | VAL | M | 196 | −19.335 | 2.919 | −22.506 | 1.00 | 30.96 | N |
| ATOM | 6552 | CA | VAL | M | 196 | −18.045 | 3.151 | −21.871 | 1.00 | 30.44 | C |
| ATOM | 6553 | CB | VAL | M | 196 | −18.219 | 3.334 | −20.361 | 1.00 | 30.34 | C |
| ATOM | 6554 | CG1 | VAL | M | 196 | −16.873 | 3.477 | −19.678 | 1.00 | 30.33 | C |
| ATOM | 6555 | CG2 | VAL | M | 196 | −19.104 | 4.522 | −20.079 | 1.00 | 30.23 | C |
| ATOM | 6556 | C | VAL | M | 196 | −17.071 | 2.007 | −22.103 | 1.00 | 30.35 | C |
| ATOM | 6557 | O | VAL | M | 196 | −17.382 | 0.847 | −21.836 | 1.00 | 30.27 | O |
| ATOM | 6558 | N | THR | M | 197 | −15.879 | 2.360 | −22.570 | 1.00 | 30.37 | N |
| ATOM | 6559 | CA | THR | M | 197 | −14.845 | 1.398 | −22.922 | 1.00 | 30.55 | C |
| ATOM | 6560 | CB | THR | M | 197 | −14.572 | 1.453 | −24.440 | 1.00 | 30.37 | C |
| ATOM | 6561 | OG1 | THR | M | 197 | −15.762 | 1.060 | −25.129 | 1.00 | 30.71 | O |
| ATOM | 6562 | CG2 | THR | M | 197 | −13.403 | 0.552 | −24.862 | 1.00 | 29.69 | C |
| ATOM | 6563 | C | THR | M | 197 | −13.580 | 1.659 | −22.090 | 1.00 | 31.03 | C |
| ATOM | 6564 | O | THR | M | 197 | −12.912 | 2.695 | −22.248 | 1.00 | 30.97 | O |
| ATOM | 6565 | N | HIS | M | 198 | −13.277 | 0.710 | −21.200 | 1.00 | 31.08 | N |
| ATOM | 6566 | CA | HIS | M | 198 | −12.196 | 0.842 | −20.220 | 1.00 | 31.25 | C |
| ATOM | 6567 | CB | HIS | M | 198 | −12.717 | 1.488 | −18.922 | 1.00 | 31.05 | C |
| ATOM | 6568 | CG | HIS | M | 198 | −11.632 | 1.884 | −17.964 | 1.00 | 30.87 | C |
| ATOM | 6569 | ND1 | HIS | M | 198 | −11.161 | 1.042 | −16.979 | 1.00 | 30.69 | N |
| ATOM | 6570 | CE1 | HIS | M | 198 | −10.206 | 1.651 | −16.299 | 1.00 | 29.77 | C |
| ATOM | 6571 | NE2 | HIS | M | 198 | −10.034 | 2.855 | −16.812 | 1.00 | 29.41 | N |
| ATOM | 6572 | CD2 | HIS | M | 198 | −10.918 | 3.029 | −17.849 | 1.00 | 30.32 | C |
| ATOM | 6573 | C | HIS | M | 198 | −11.550 | −0.514 | −19.918 | 1.00 | 31.19 | C |
| ATOM | 6574 | O | HIS | M | 198 | −12.207 | −1.553 | −19.967 | 1.00 | 31.29 | O |
| ATOM | 6575 | N | GLN | M | 199 | −10.262 | −0.480 | −19.596 | 1.00 | 31.22 | N |
| ATOM | 6576 | CA | GLN | M | 199 | −9.468 | −1.670 | −19.300 | 1.00 | 31.48 | C |
| ATOM | 6577 | CB | GLN | M | 199 | −8.048 | −1.243 | −18.939 | 1.00 | 31.25 | C |
| ATOM | 6578 | CG | GLN | M | 199 | −7.040 | −2.344 | −19.019 | 1.00 | 31.90 | C |
| ATOM | 6579 | CD | GLN | M | 199 | −5.629 | −1.882 | −18.714 | 1.00 | 32.55 | C |
| ATOM | 6580 | OE1 | GLN | M | 199 | −5.366 | −0.691 | −18.532 | 1.00 | 31.66 | O |
| ATOM | 6581 | NE2 | GLN | M | 199 | −4.704 | −2.838 | −18.667 | 1.00 | 33.14 | N |
| ATOM | 6582 | C | GLN | M | 199 | −10.052 | −2.537 | −18.168 | 1.00 | 31.62 | C |
| ATOM | 6583 | O | GLN | M | 199 | −9.896 | −3.751 | −18.164 | 1.00 | 31.55 | O |
| ATOM | 6584 | N | GLY | M | 200 | −10.723 | −1.902 | −17.218 | 1.00 | 31.86 | N |
| ATOM | 6585 | CA | GLY | M | 200 | −11.284 | −2.597 | −16.068 | 1.00 | 32.43 | C |
| ATOM | 6586 | C | GLY | M | 200 | −12.580 | −3.319 | −16.349 | 1.00 | 32.93 | C |
| ATOM | 6587 | O | GLY | M | 200 | −13.053 | −4.089 | −15.514 | 1.00 | 32.76 | O |
| ATOM | 6588 | N | LEU | M | 201 | −13.162 | −3.062 | −17.521 | 1.00 | 33.77 | N |
| ATOM | 6589 | CA | LEU | M | 201 | −14.371 | −3.759 | −17.968 | 1.00 | 34.19 | C |
| ATOM | 6590 | CB | LEU | M | 201 | −15.362 | −2.769 | −18.571 | 1.00 | 33.84 | C |
| ATOM | 6591 | CG | LEU | M | 201 | −15.889 | −1.678 | −17.647 | 1.00 | 32.89 | C |
| ATOM | 6592 | CD1 | LEU | M | 201 | −16.569 | −0.603 | −18.479 | 1.00 | 32.29 | C |
| ATOM | 6593 | CD2 | LEU | M | 201 | −16.827 | −2.242 | −16.582 | 1.00 | 31.76 | C |
| ATOM | 6594 | C | LEU | M | 201 | −13.999 | −4.811 | −18.995 | 1.00 | 34.87 | C |
| ATOM | 6595 | O | LEU | M | 201 | −13.176 | −4.547 | −19.872 | 1.00 | 35.11 | O |
| ATOM | 6596 | N | SER | M | 202 | −14.588 | −6.003 | −18.889 | 1.00 | 35.87 | N |
| ATOM | 6597 | CA | SER | M | 202 | −14.283 | −7.082 | −19.853 | 1.00 | 36.55 | C |
| ATOM | 6598 | CB | SER | M | 202 | −14.740 | −8.459 | −19.353 | 1.00 | 36.21 | C |
| ATOM | 6599 | OG | SER | M | 202 | −16.063 | −8.421 | −18.858 | 1.00 | 36.66 | O |
| ATOM | 6600 | C | SER | M | 202 | −14.842 | −6.761 | −21.241 | 1.00 | 36.93 | C |
| ATOM | 6601 | O | SER | M | 202 | −14.225 | −7.085 | −22.263 | 1.00 | 37.11 | O |
| ATOM | 6602 | N | SER | M | 203 | −15.997 | −6.105 | −21.272 | 1.00 | 37.24 | N |
| ATOM | 6603 | CA | SER | M | 203 | −16.517 | −5.548 | −22.518 | 1.00 | 37.86 | C |
| ATOM | 6604 | CB | SER | M | 203 | −17.399 | −6.572 | −23.261 | 1.00 | 37.80 | C |
| ATOM | 6605 | OG | SER | M | 203 | −18.666 | −6.719 | −22.639 | 1.00 | 38.53 | O |
| ATOM | 6606 | C | SER | M | 203 | −17.263 | −4.236 | −22.233 | 1.00 | 37.86 | C |
| ATOM | 6607 | O | SER | M | 203 | −17.666 | −4.003 | −21.090 | 1.00 | 38.05 | O |
| ATOM | 6608 | N | PRO | M | 204 | −17.433 | −3.373 | −23.262 | 1.00 | 38.00 | N |
| ATOM | 6609 | CA | PRO | M | 204 | −18.098 | −2.078 | −23.086 | 1.00 | 38.10 | C |
| ATOM | 6610 | CB | PRO | M | 204 | −18.340 | −1.617 | −24.524 | 1.00 | 38.04 | C |
| ATOM | 6611 | CG | PRO | M | 204 | −17.192 | −2.168 | −25.272 | 1.00 | 38.16 | C |
| ATOM | 6612 | CD | PRO | M | 204 | −16.861 | −3.497 | −24.620 | 1.00 | 38.03 | C |
| ATOM | 6613 | C | PRO | M | 204 | −19.419 | −2.150 | −22.323 | 1.00 | 38.15 | C |
| ATOM | 6614 | O | PRO | M | 204 | −20.258 | −3.005 | −22.610 | 1.00 | 38.35 | O |
| ATOM | 6615 | N | VAL | M | 205 | −19.575 | −1.257 | −21.350 | 1.00 | 38.12 | N |
| ATOM | 6616 | CA | VAL | M | 205 | −20.796 | −1.149 | −20.566 | 1.00 | 38.24 | C |
| ATOM | 6617 | CB | VAL | M | 205 | −20.501 | −0.965 | −19.041 | 1.00 | 38.30 | C |
| ATOM | 6618 | CG1 | VAL | M | 205 | −21.718 | −0.446 | −18.278 | 1.00 | 37.50 | C |
| ATOM | 6619 | CG2 | VAL | M | 205 | −20.024 | −2.281 | −18.432 | 1.00 | 38.75 | C |
| ATOM | 6620 | C | VAL | M | 205 | −21.609 | 0.011 | −21.102 | 1.00 | 38.34 | C |
| ATOM | 6621 | O | VAL | M | 205 | −21.062 | 1.012 | −21.560 | 1.00 | 38.18 | O |
| ATOM | 6622 | N | THR | M | 206 | −22.922 | −0.145 | −21.035 | 1.00 | 38.73 | N |
| ATOM | 6623 | CA | THR | M | 206 | −23.847 | 0.837 | −21.541 | 1.00 | 38.95 | C |
| ATOM | 6624 | CB | THR | M | 206 | −24.525 | 0.357 | −22.849 | 1.00 | 38.91 | C |
| ATOM | 6625 | OG1 | THR | M | 206 | −23.537 | 0.231 | −23.884 | 1.00 | 38.81 | O |
| ATOM | 6626 | CG2 | THR | M | 206 | −25.608 | 1.341 | −23.296 | 1.00 | 38.76 | C |
| ATOM | 6627 | C | THR | M | 206 | −24.895 | 1.114 | −20.489 | 1.00 | 39.14 | C |
| ATOM | 6628 | O | THR | M | 206 | −25.601 | 0.207 | −20.053 | 1.00 | 39.11 | O |
| ATOM | 6629 | N | LYS | M | 207 | −24.952 | 2.372 | −20.068 | 1.00 | 39.50 | N |
| ATOM | 6630 | CA | LYS | M | 207 | −26.061 | 2.889 | −19.297 | 1.00 | 40.06 | C |
| ATOM | 6631 | CB | LYS | M | 207 | −25.569 | 3.695 | −18.098 | 1.00 | 40.26 | C |
| ATOM | 6632 | CG | LYS | M | 207 | −24.897 | 2.859 | −17.008 | 1.00 | 41.23 | C |
| ATOM | 6633 | CD | LYS | M | 207 | −25.904 | 2.185 | −16.086 | 1.00 | 42.52 | C |
| ATOM | 6634 | CE | LYS | M | 207 | −25.197 | 1.201 | −15.145 | 1.00 | 44.49 | C |
| ATOM | 6635 | NZ | LYS | M | 207 | −26.010 | 0.935 | −13.912 | 1.00 | 43.70 | N |
| ATOM | 6636 | C | LYS | M | 207 | −26.870 | 3.763 | −20.236 | 1.00 | 40.53 | C |
| ATOM | 6637 | O | LYS | M | 207 | −26.311 | 4.490 | −21.075 | 1.00 | 40.25 | O |
| ATOM | 6638 | N | SER | M | 208 | −28.190 | 3.677 | −20.103 | 1.00 | 41.19 | N |
| ATOM | 6639 | CA | SER | M | 208 | −29.099 | 4.319 | −21.045 | 1.00 | 41.78 | C |
| ATOM | 6640 | CB | SER | M | 208 | −29.272 | 3.454 | −22.298 | 1.00 | 41.99 | C |
| ATOM | 6641 | OG | SER | M | 208 | −29.913 | 2.232 | −21.984 | 1.00 | 42.61 | O |
| ATOM | 6642 | C | SER | M | 208 | −30.459 | 4.616 | −20.436 | 1.00 | 41.93 | C |
| ATOM | 6643 | O | SER | M | 208 | −30.811 | 4.084 | −19.392 | 1.00 | 41.61 | O |
| ATOM | 6644 | N | PHE | M | 209 | −31.204 | 5.495 | −21.097 | 1.00 | 42.79 | N |
| ATOM | 6645 | CA | PHE | M | 209 | −32.569 | 5.817 | −20.697 | 1.00 | 43.53 | C |
| ATOM | 6646 | CB | PHE | M | 209 | −32.605 | 6.983 | −19.694 | 1.00 | 43.42 | C |
| ATOM | 6647 | CG | PHE | M | 209 | −32.187 | 8.316 | −20.278 | 1.00 | 43.78 | C |
| ATOM | 6648 | CD1 | PHE | M | 209 | −33.135 | 9.210 | −20.763 | 1.00 | 43.68 | C |
| ATOM | 6649 | CE1 | PHE | M | 209 | −32.755 | 10.430 | −21.295 | 1.00 | 43.43 | C |
| ATOM | 6650 | CZ | PHE | M | 209 | −31.417 | 10.777 | −21.341 | 1.00 | 43.50 | C |
| ATOM | 6651 | CE2 | PHE | M | 209 | −30.464 | 9.905 | −20.862 | 1.00 | 43.64 | C |
| ATOM | 6652 | CD2 | PHE | M | 209 | −30.848 | 8.679 | −20.331 | 1.00 | 44.17 | C |
| ATOM | 6653 | C | PHE | M | 209 | −33.427 | 6.122 | −21.919 | 1.00 | 44.07 | C |
| ATOM | 6654 | O | PHE | M | 209 | −32.913 | 6.420 | −23.005 | 1.00 | 43.88 | O |
| ATOM | 6655 | N | ASN | M | 210 | −34.736 | 6.027 | −21.723 | 1.00 | 45.07 | N |
| ATOM | 6656 | CA | ASN | M | 210 | −35.708 | 6.387 | −22.739 | 1.00 | 46.26 | C |
| ATOM | 6657 | CB | ASN | M | 210 | −36.689 | 5.232 | −22.975 | 1.00 | 46.13 | C |
| ATOM | 6658 | CG | ASN | M | 210 | −36.029 | 4.023 | −23.632 | 1.00 | 45.71 | C |
| ATOM | 6659 | OD1 | ASN | M | 210 | −35.125 | 4.161 | −24.460 | 1.00 | 45.64 | O |
| ATOM | 6660 | ND2 | ASN | M | 210 | −36.489 | 2.833 | −23.271 | 1.00 | 44.10 | N |
| ATOM | 6661 | C | ASN | M | 210 | −36.436 | 7.664 | −22.329 | 1.00 | 47.16 | C |
| ATOM | 6662 | O | ASN | M | 210 | −36.990 | 7.747 | −21.224 | 1.00 | 47.30 | O |
| ATOM | 6663 | N | ARG | M | 211 | −36.419 | 8.646 | −23.226 | 1.00 | 48.19 | N |
| ATOM | 6664 | CA | ARG | M | 211 | −36.936 | 10.001 | −22.972 | 1.00 | 49.71 | C |
| ATOM | 6665 | CB | ARG | M | 211 | −37.065 | 10.755 | −24.302 | 1.00 | 49.43 | C |
| ATOM | 6666 | CG | ARG | M | 211 | −37.330 | 12.241 | −24.171 | 1.00 | 49.48 | C |
| ATOM | 6667 | CD | ARG | M | 211 | −37.206 | 12.968 | −25.507 | 1.00 | 49.35 | C |
| ATOM | 6668 | NE | ARG | M | 211 | −37.889 | 12.278 | −26.602 | 1.00 | 50.41 | N |
| ATOM | 6669 | CZ | ARG | M | 211 | −39.207 | 12.275 | −26.797 | 1.00 | 50.69 | C |
| ATOM | 6670 | NH1 | ARG | M | 211 | −40.014 | 12.922 | −25.960 | 1.00 | 50.48 | N |
| ATOM | 6671 | NH2 | ARG | M | 211 | −39.718 | 11.607 | −27.828 | 1.00 | 49.85 | N |
| ATOM | 6672 | C | ARG | M | 211 | −38.248 | 10.084 | −22.154 | 1.00 | 50.95 | C |
| ATOM | 6673 | O | ARG | M | 211 | −39.283 | 9.536 | −22.549 | 1.00 | 50.86 | O |
| ATOM | 6674 | N | GLY | M | 212 | −38.175 | 10.766 | −21.009 | 1.00 | 52.37 | N |
| ATOM | 6675 | CA | GLY | M | 212 | −39.328 | 10.965 | −20.125 | 1.00 | 54.20 | C |
| ATOM | 6676 | C | GLY | M | 212 | −39.904 | 9.697 | −19.504 | 1.00 | 55.46 | C |
| ATOM | 6677 | O | GLY | M | 212 | −40.955 | 9.203 | −19.939 | 1.00 | 55.64 | O |
| ATOM | 6678 | N | GLU | M | 213 | −39.217 | 9.170 | −18.489 | 1.00 | 56.29 | N |
| ATOM | 6679 | CA | GLU | M | 213 | −39.722 | 8.046 | −17.696 | 1.00 | 57.10 | C |
| ATOM | 6680 | CB | GLU | M | 213 | −39.138 | 6.722 | −18.198 | 1.00 | 57.36 | C |
| ATOM | 6681 | CG | GLU | M | 213 | −39.896 | 6.084 | −19.368 | 1.00 | 58.89 | C |
| ATOM | 6682 | CD | GLU | M | 213 | −39.333 | 4.712 | −19.765 | 1.00 | 61.34 | C |
| ATOM | 6683 | OE1 | GLU | M | 213 | −38.939 | 3.924 | −18.870 | 1.00 | 61.94 | O |
| ATOM | 6684 | OE2 | GLU | M | 213 | −39.288 | 4.416 | −20.982 | 1.00 | 61.91 | O |
| ATOM | 6685 | C | GLU | M | 213 | −39.406 | 8.243 | −16.206 | 1.00 | 57.30 | C |
| ATOM | 6686 | O | GLU | M | 213 | −39.968 | 7.572 | −15.333 | 1.00 | 57.40 | O |
| ATOM | 6687 | O | HOH | S | 1 | 15.826 | 40.047 | −27.099 | 1.00 | 14.46 | O |
| ATOM | 6688 | O | HOH | S | 2 | 25.830 | 13.982 | 43.032 | 1.00 | 26.91 | O |
| ATOM | 6689 | O | HOH | S | 3 | −9.456 | 42.548 | −16.077 | 1.00 | 26.59 | O |
| ATOM | 6690 | O | HOH | S | 4 | 16.817 | 28.304 | 24.273 | 1.00 | 25.58 | O |
| ATOM | 6691 | O | HOH | S | 5 | −13.840 | 9.389 | −24.969 | 1.00 | 21.41 | O |
| ATOM | 6692 | O | HOH | S | 6 | 32.735 | 25.718 | 33.868 | 1.00 | 33.88 | O |
| ATOM | 6693 | O | HOH | S | 7 | 35.042 | 27.660 | 13.058 | 1.00 | 30.35 | O |
| ATOM | 6694 | O | HOH | S | 8 | 5.145 | 14.417 | 22.919 | 1.00 | 30.63 | O |
| ATOM | 6695 | O | HOH | S | 9 | −14.583 | 32.449 | −24.155 | 1.00 | 22.40 | O |
| ATOM | 6696 | O | HOH | S | 10 | 17.526 | 22.468 | 27.850 | 1.00 | 15.65 | O |
| ATOM | 6697 | O | HOH | S | 11 | −4.598 | 43.261 | −9.264 | 1.00 | 22.93 | O |
| ATOM | 6698 | O | HOH | S | 12 | 5.309 | 27.267 | 15.486 | 1.00 | 25.45 | O |
| ATOM | 6699 | O | HOH | S | 13 | −11.639 | 38.022 | 26.051 | 1.00 | 17.78 | O |
| ATOM | 6700 | O | HOH | S | 14 | 2.366 | 29.641 | 6.623 | 1.00 | 16.56 | O |
| ATOM | 6701 | O | HOH | S | 15 | −25.136 | 30.285 | 18.940 | 1.00 | 14.78 | O |
| ATOM | 6702 | O | HOH | S | 16 | 3.589 | 3.188 | 21.860 | 1.00 | 34.44 | O |
| ATOM | 6703 | O | HOH | S | 17 | −2.373 | 23.819 | 9.201 | 1.00 | 23.03 | O |
| ATOM | 6704 | O | HOH | S | 18 | 15.404 | 15.918 | 11.841 | 1.00 | 16.44 | O |
| ATOM | 6705 | O | HOH | S | 19 | −1.613 | 11.482 | −6.034 | 1.00 | 13.73 | O |
| ATOM | 6706 | O | HOH | S | 20 | 3.471 | 15.781 | −19.593 | 1.00 | 18.26 | O |
| ATOM | 6707 | O | HOH | S | 21 | −24.001 | 16.246 | 4.341 | 1.00 | 33.62 | O |
| ATOM | 6708 | O | HOH | S | 22 | −18.262 | −0.869 | −11.191 | 1.00 | 28.67 | O |
| ATOM | 6709 | O | HOH | S | 23 | 2.597 | 7.870 | 23.005 | 1.00 | 21.37 | O |
| ATOM | 6710 | O | HOH | S | 24 | 14.597 | 43.217 | 29.216 | 1.00 | 31.68 | O |
| ATOM | 6711 | O | HOH | S | 25 | 24.417 | 43.479 | 28.352 | 1.00 | 25.09 | O |
| ATOM | 6712 | O | HOH | S | 26 | −37.211 | 9.010 | −8.190 | 1.00 | 33.96 | O |
| ATOM | 6713 | O | HOH | S | 27 | 16.462 | 18.298 | −28.383 | 1.00 | 27.70 | O |
| ATOM | 6714 | O | HOH | S | 28 | 0.069 | 11.670 | −3.992 | 1.00 | 25.39 | O |
| ATOM | 6715 | O | HOH | S | 29 | −24.065 | 35.323 | 9.218 | 1.00 | 18.55 | O |
| ATOM | 6716 | O | HOH | S | 30 | −9.219 | 49.251 | 23.098 | 1.00 | 24.89 | O |
| ATOM | 6717 | O | HOH | S | 31 | 23.282 | 2.819 | 33.005 | 1.00 | 19.57 | O |
| ATOM | 6718 | O | HOH | S | 32 | 9.329 | 38.803 | 7.759 | 1.00 | 14.83 | O |
| ATOM | 6719 | O | HOH | S | 33 | 16.221 | 15.356 | 30.727 | 1.00 | 23.79 | O |
| ATOM | 6720 | O | HOH | S | 34 | −11.073 | 40.334 | −13.843 | 1.00 | 18.47 | O |
| ATOM | 6721 | O | HOH | S | 35 | 16.017 | 34.598 | −12.684 | 1.00 | 15.98 | O |
| ATOM | 6722 | O | HOH | S | 36 | −14.161 | 43.074 | 5.652 | 1.00 | 12.91 | O |
| ATOM | 6723 | O | HOH | S | 37 | 16.314 | 12.187 | 9.315 | 1.00 | 25.11 | O |
| ATOM | 6724 | O | HOH | S | 38 | 19.459 | 23.928 | 9.007 | 1.00 | 21.12 | O |
| ATOM | 6725 | O | HOH | S | 39 | −30.131 | 28.405 | −11.926 | 1.00 | 33.55 | O |
| ATOM | 6726 | O | HOH | S | 40 | −9.199 | 34.719 | 18.655 | 1.00 | 9.26 | O |
| ATOM | 6727 | O | HOH | S | 41 | 29.962 | 29.647 | −8.922 | 1.00 | 21.86 | O |
| ATOM | 6728 | O | HOH | S | 42 | −19.881 | 22.807 | 7.792 | 1.00 | 22.81 | O |
| ATOM | 6729 | O | HOH | S | 43 | 26.689 | 20.239 | 39.153 | 1.00 | 21.99 | O |
| ATOM | 6730 | O | HOH | S | 44 | 15.972 | 13.148 | 3.624 | 1.00 | 25.80 | O |
| ATOM | 6731 | O | HOH | S | 45 | −19.659 | 10.263 | −28.567 | 1.00 | 23.43 | O |
| ATOM | 6732 | O | HOH | S | 46 | −13.881 | 16.875 | −24.718 | 1.00 | 15.59 | O |
| ATOM | 6733 | O | HOH | S | 47 | −2.204 | 41.013 | −5.838 | 1.00 | 18.73 | O |
| ATOM | 6734 | O | HOH | S | 48 | −9.610 | 44.420 | 24.625 | 1.00 | 17.98 | O |
| ATOM | 6735 | O | HOH | S | 49 | −20.885 | 32.397 | 6.869 | 1.00 | 29.57 | O |
| ATOM | 6736 | O | HOH | S | 50 | −26.393 | 23.986 | 1.793 | 1.00 | 33.37 | O |
| ATOM | 6737 | O | HOH | S | 51 | 5.933 | 46.639 | −19.670 | 1.00 | 19.74 | O |
| ATOM | 6738 | O | HOH | S | 52 | 13.810 | 21.529 | 21.528 | 1.00 | 19.90 | O |
| ATOM | 6739 | O | HOH | S | 53 | 5.068 | 52.780 | −12.613 | 1.00 | 32.70 | O |
| ATOM | 6740 | O | HOH | S | 54 | −10.716 | 31.995 | −24.566 | 1.00 | 17.98 | O |
| ATOM | 6741 | O | HOH | S | 55 | 15.063 | 38.411 | 14.313 | 1.00 | 19.14 | O |
| ATOM | 6742 | O | HOH | S | 56 | 13.930 | 15.390 | 27.725 | 1.00 | 28.15 | O |
| ATOM | 6743 | O | HOH | S | 57 | −3.349 | −0.727 | −5.908 | 1.00 | 32.41 | O |
| ATOM | 6744 | O | HOH | S | 58 | 3.738 | 52.209 | 20.460 | 1.00 | 14.07 | O |
| ATOM | 6745 | O | HOH | S | 59 | 37.665 | 21.936 | 7.965 | 1.00 | 31.11 | O |
| ATOM | 6746 | O | HOH | S | 60 | 12.474 | 23.410 | −4.060 | 1.00 | 27.96 | O |
| ATOM | 6747 | O | HOH | S | 61 | −11.973 | 32.484 | −18.034 | 1.00 | 23.60 | O |
| ATOM | 6748 | O | HOH | S | 62 | 24.234 | 18.549 | −3.734 | 1.00 | 35.52 | O |
| ATOM | 6749 | O | HOH | S | 63 | 22.328 | 16.589 | 34.335 | 1.00 | 25.42 | O |
| ATOM | 6750 | O | HOH | S | 64 | 8.835 | 34.928 | 8.333 | 1.00 | 25.65 | O |
| ATOM | 6751 | O | HOH | S | 65 | −2.981 | 38.088 | 27.181 | 1.00 | 25.54 | O |
| ATOM | 6752 | O | HOH | S | 66 | −10.576 | 28.538 | −21.186 | 1.00 | 18.78 | O |
| ATOM | 6753 | O | HOH | S | 67 | 33.756 | 27.003 | 22.488 | 1.00 | 34.34 | O |
| ATOM | 6754 | O | HOH | S | 68 | −17.907 | 41.735 | 20.353 | 1.00 | 22.20 | O |
| ATOM | 6755 | O | HOH | S | 69 | −24.084 | 4.576 | −36.425 | 1.00 | 36.12 | O |
| ATOM | 6756 | O | HOH | S | 70 | 20.921 | 16.272 | −14.571 | 1.00 | 24.97 | O |
| ATOM | 6757 | O | HOH | S | 71 | 1.522 | 52.096 | 14.258 | 1.00 | 9.17 | O |
| ATOM | 6758 | O | HOH | S | 72 | −9.646 | 39.670 | 11.183 | 1.00 | 16.25 | O |
| ATOM | 6759 | O | HOH | S | 73 | −2.954 | 36.732 | −9.804 | 1.00 | 25.67 | O |
| ATOM | 6760 | O | HOH | S | 74 | −3.265 | 36.095 | −6.749 | 1.00 | 30.95 | O |
| ATOM | 6761 | O | HOH | S | 75 | −10.402 | 21.965 | 28.628 | 1.00 | 30.14 | O |
| ATOM | 6762 | O | HOH | S | 76 | 15.877 | 46.390 | −22.849 | 1.00 | 27.08 | O |
| ATOM | 6763 | O | HOH | S | 77 | −15.131 | 29.276 | 3.536 | 1.00 | 22.53 | O |
| ATOM | 6764 | O | HOH | S | 78 | −16.618 | 23.333 | 18.319 | 1.00 | 15.84 | O |
| ATOM | 6765 | O | HOH | S | 79 | −20.482 | 21.108 | −23.361 | 1.00 | 16.95 | O |
| ATOM | 6766 | O | HOH | S | 80 | −19.075 | 44.594 | 13.434 | 1.00 | 17.50 | O |
| ATOM | 6767 | O | HOH | S | 81 | −7.889 | 43.835 | 26.896 | 1.00 | 16.71 | O |
| ATOM | 6768 | O | HOH | S | 82 | 15.256 | 27.868 | −18.398 | 1.00 | 19.05 | O |
| ATOM | 6769 | O | HOH | S | 83 | −18.471 | 20.507 | −0.402 | 1.00 | 20.76 | O |
| ATOM | 6770 | O | HOH | S | 84 | −21.491 | 44.932 | 12.783 | 1.00 | 24.01 | O |
| ATOM | 6771 | O | HOH | S | 85 | −7.756 | 25.828 | 6.285 | 1.00 | 17.65 | O |
| ATOM | 6772 | O | HOH | S | 86 | 1.689 | 43.115 | −8.087 | 1.00 | 20.44 | O |
| ATOM | 6773 | O | HOH | S | 87 | 8.394 | 47.078 | −2.021 | 1.00 | 22.87 | O |
| ATOM | 6774 | O | HOH | S | 88 | −17.518 | 19.520 | −16.638 | 1.00 | 28.24 | O |
| ATOM | 6775 | O | HOH | S | 89 | −10.316 | 12.141 | −8.978 | 1.00 | 16.80 | O |
| ATOM | 6776 | O | HOH | S | 90 | 31.121 | 30.454 | −13.481 | 1.00 | 20.83 | O |
| ATOM | 6777 | O | HOH | S | 91 | 23.977 | 22.050 | −3.444 | 1.00 | 27.82 | O |
| ATOM | 6778 | O | HOH | S | 92 | 10.636 | 37.419 | 9.722 | 1.00 | 14.99 | O |
| ATOM | 6779 | O | HOH | S | 93 | 0.734 | 47.901 | 29.315 | 1.00 | 26.11 | O |
| ATOM | 6780 | O | HOH | S | 94 | 9.298 | 43.455 | −27.276 | 1.00 | 41.47 | O |
| ATOM | 6781 | O | HOH | S | 95 | −11.849 | 42.071 | 7.004 | 1.00 | 22.58 | O |
| ATOM | 6782 | O | HOH | S | 96 | −13.277 | 14.716 | 1.878 | 1.00 | 16.89 | O |
| ATOM | 6783 | O | HOH | S | 97 | 7.455 | 23.167 | −21.281 | 1.00 | 27.80 | O |
| ATOM | 6784 | O7 | LPA | A | 1 | −16.037 | 37.078 | 14.655 | 1.00 | 20.15 | O |
| ATOM | 6785 | C4 | LPA | A | 1 | −15.997 | 38.193 | 14.158 | 1.00 | 19.46 | C |
| ATOM | 6786 | O6 | LPA | A | 1 | −16.543 | 38.331 | 12.829 | 1.00 | 19.06 | O |
| ATOM | 6787 | C3 | LPA | A | 1 | −17.564 | 37.450 | 12.381 | 1.00 | 18.83 | C |
| ATOM | 6788 | C2 | LPA | A | 1 | −18.120 | 38.043 | 11.099 | 1.00 | 18.44 | C |
| ATOM | 6789 | O5 | LPA | A | 1 | −17.073 | 38.072 | 10.123 | 1.00 | 19.60 | O |
| ATOM | 6790 | C1 | LPA | A | 1 | −19.280 | 37.212 | 10.579 | 1.00 | 17.57 | C |
| ATOM | 6791 | O4 | LPA | A | 1 | −20.239 | 37.114 | 11.622 | 1.00 | 16.76 | O |
| ATOM | 6792 | P1 | LPA | A | 1 | −21.608 | 36.320 | 11.389 | 1.00 | 16.02 | P |
| ATOM | 6793 | O3 | LPA | A | 1 | −22.310 | 36.578 | 12.691 | 1.00 | 14.82 | O |
| ATOM | 6794 | O2 | LPA | A | 1 | −22.208 | 36.934 | 10.156 | 1.00 | 16.89 | O |
| ATOM | 6795 | O1 | LPA | A | 1 | −21.221 | 34.893 | 11.129 | 1.00 | 16.42 | O |
| ATOM | 6796 | C5 | LPA | A | 1 | −15.391 | 39.375 | 14.895 | 1.00 | 19.49 | C |
| ATOM | 6797 | C6 | LPA | A | 1 | −15.841 | 40.724 | 14.336 | 1.00 | 20.29 | C |
| ATOM | 6798 | C7 | LPA | A | 1 | −15.219 | 41.896 | 15.087 | 1.00 | 21.96 | C |
| ATOM | 6799 | C8 | LPA | A | 1 | −15.816 | 43.211 | 14.603 | 1.00 | 25.79 | C |
| ATOM | 6800 | C9 | LPA | A | 1 | −15.134 | 44.446 | 15.182 | 1.00 | 29.23 | C |
| ATOM | 6801 | C10 | LPA | A | 1 | −14.985 | 45.557 | 14.137 | 1.00 | 32.95 | C |
| ATOM | 6802 | C11 | LPA | A | 1 | −15.373 | 46.920 | 14.713 | 1.00 | 36.95 | C |
| ATOM | 6803 | C12 | LPA | A | 1 | −14.993 | 48.049 | 13.774 | 1.00 | 39.43 | C |
| ATOM | 6804 | C13 | LPA | A | 1 | −15.799 | 49.068 | 13.426 | 1.00 | 42.45 | C |
| ATOM | 6805 | C14 | LPA | A | 1 | −17.235 | 49.294 | 13.879 | 1.00 | 44.42 | C |
| ATOM | 6806 | C15 | LPA | A | 1 | −18.235 | 48.447 | 13.093 | 1.00 | 47.58 | C |
| ATOM | 6807 | C16 | LPA | A | 1 | −19.149 | 48.868 | 12.189 | 1.00 | 48.88 | C |
| ATOM | 6808 | C17 | LPA | A | 1 | −19.364 | 50.305 | 11.762 | 1.00 | 50.01 | C |
| ATOM | 6809 | C18 | LPA | A | 1 | −19.135 | 50.440 | 10.254 | 1.00 | 51.51 | C |
| ATOM | 6810 | C19 | LPA | A | 1 | −17.944 | 51.351 | 9.934 | 1.00 | 52.25 | C |
| ATOM | 6811 | C20 | LPA | A | 1 | −18.250 | 52.341 | 8.808 | 1.00 | 52.75 | C |
| ATOM | 6812 | C21 | LPA | A | 1 | −17.103 | 52.429 | 7.815 | 1.00 | 52.55 | C |
| ATOM | 6813 | O7 | LPA | B | 1 | 22.100 | 30.313 | −15.311 | 1.00 | 22.39 | O |
| ATOM | 6814 | C4 | LPA | B | 1 | 22.216 | 31.509 | −15.105 | 1.00 | 21.54 | C |
| ATOM | 6815 | O6 | LPA | B | 1 | 22.762 | 31.953 | −13.846 | 1.00 | 21.03 | O |
| ATOM | 6816 | C3 | LPA | B | 1 | 23.747 | 31.220 | −13.123 | 1.00 | 19.84 | C |
| ATOM | 6817 | C2 | LPA | B | 1 | 24.325 | 32.196 | −12.102 | 1.00 | 19.11 | C |
| ATOM | 6818 | O5 | LPA | B | 1 | 23.268 | 32.626 | −11.227 | 1.00 | 17.80 | O |
| ATOM | 6819 | C1 | LPA | B | 1 | 25.449 | 31.548 | −11.303 | 1.00 | 19.76 | C |
| ATOM | 6820 | O4 | LPA | B | 1 | 26.404 | 30.985 | −12.192 | 1.00 | 20.88 | O |
| ATOM | 6821 | P1 | LPA | B | 1 | 27.604 | 30.051 | −11.656 | 1.00 | 23.12 | P |
| ATOM | 6822 | O3 | LPA | B | 1 | 28.483 | 29.997 | −12.884 | 1.00 | 24.03 | O |
| ATOM | 6823 | O2 | LPA | B | 1 | 28.221 | 30.798 | −10.501 | 1.00 | 22.11 | O |
| ATOM | 6824 | O1 | LPA | B | 1 | 26.979 | 28.730 | −11.273 | 1.00 | 21.20 | O |
| ATOM | 6825 | C5 | LPA | B | 1 | 21.781 | 32.517 | −16.132 | 1.00 | 24.02 | C |
| ATOM | 6826 | C6 | LPA | B | 1 | 22.471 | 33.865 | −15.948 | 1.00 | 26.66 | C |
| ATOM | 6827 | C7 | LPA | B | 1 | 21.666 | 34.969 | −16.626 | 1.00 | 27.87 | C |
| ATOM | 6828 | C8 | LPA | B | 1 | 22.461 | 36.246 | −16.899 | 1.00 | 30.34 | C |
| ATOM | 6829 | C9 | LPA | B | 1 | 21.696 | 37.131 | −17.886 | 1.00 | 31.69 | C |
| ATOM | 6830 | C10 | LPA | B | 1 | 22.392 | 38.436 | −18.233 | 1.00 | 34.58 | C |
| ATOM | 6831 | C11 | LPA | B | 1 | 22.016 | 39.551 | −17.256 | 1.00 | 39.02 | C |
| ATOM | 6832 | C12 | LPA | B | 1 | 21.898 | 40.860 | −18.018 | 1.00 | 42.34 | C |
| ATOM | 6833 | C13 | LPA | B | 1 | 22.455 | 42.042 | −17.702 | 1.00 | 44.02 | C |
| ATOM | 6834 | C14 | LPA | B | 1 | 23.328 | 42.333 | −16.499 | 1.00 | 46.05 | C |
| ATOM | 6835 | C15 | LPA | B | 1 | 24.792 | 42.348 | −16.916 | 1.00 | 48.07 | C |
| ATOM | 6836 | C16 | LPA | B | 1 | 25.540 | 43.458 | −17.059 | 1.00 | 48.19 | C |
| ATOM | 6837 | C17 | LPA | B | 1 | 25.027 | 44.871 | −16.847 | 1.00 | 47.25 | C |
| ATOM | 6838 | C18 | LPA | B | 1 | 25.449 | 45.745 | −18.027 | 1.00 | 46.09 | C |
| ATOM | 6839 | C19 | LPA | B | 1 | 24.544 | 45.562 | −19.243 | 1.00 | 45.59 | C |
| ATOM | 6840 | C20 | LPA | B | 1 | 23.913 | 46.879 | −19.679 | 1.00 | 45.29 | C |
| ATOM | 6841 | C21 | LPA | B | 1 | 22.480 | 46.672 | −20.122 | 1.00 | 45.05 | C |

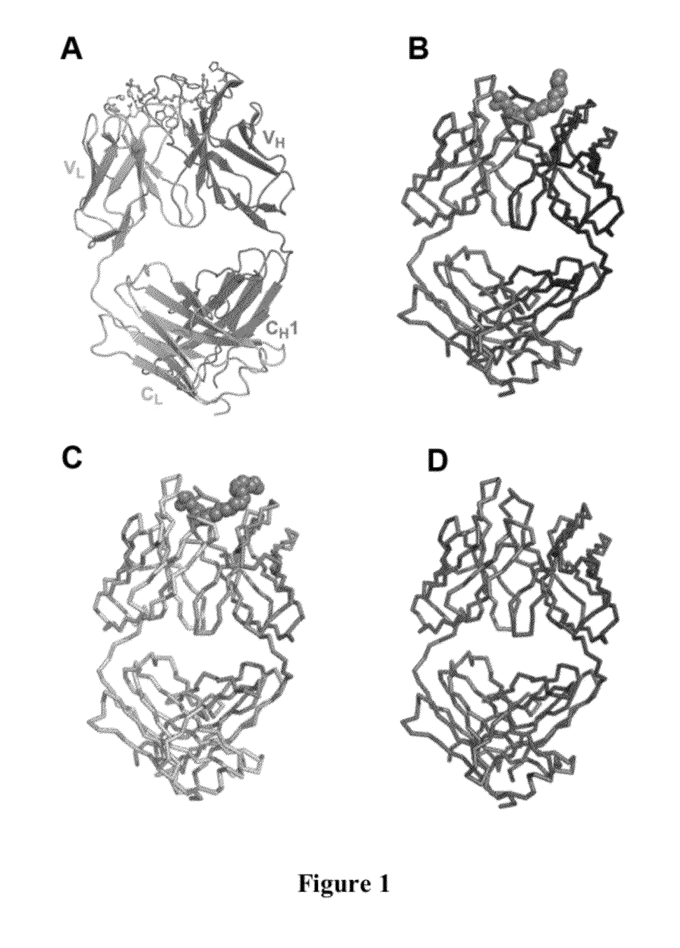

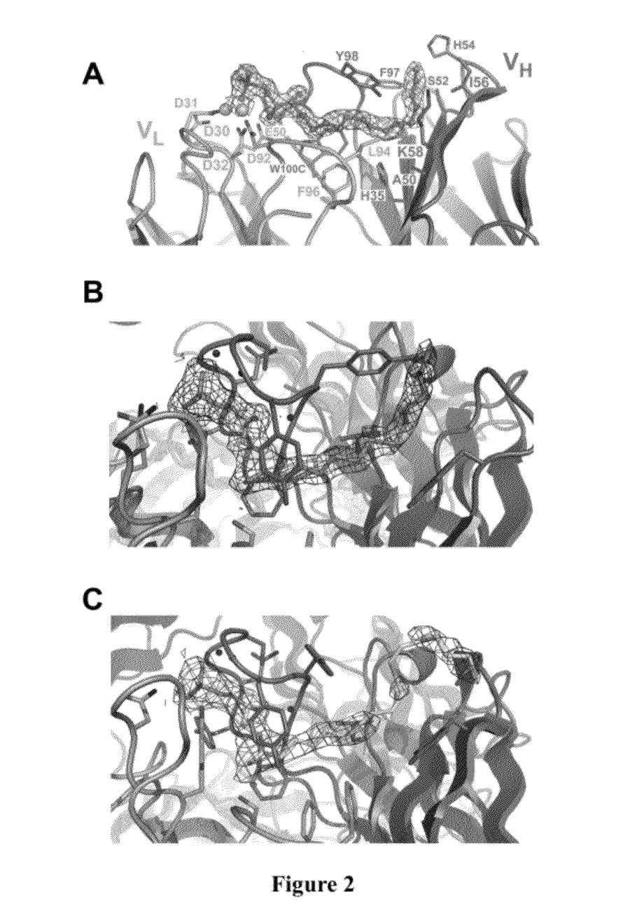

Overall structures of the lipid-Fab complexes. The structures of the Fab fragments from LT3015 exhibit the standard IgG domain folds, and the bound lipids are stabilized through interactions with amino acids in the complementary-determine regions (CDRs) of the heavy and light chains (FIG. 1). This figure shows crystal structures of the (A) 1.9 Å S1P-FAb complex (for comparison), (B) 2.0 Å 14:0 LPA-Fab complex, (C) 2.5 Å 18:2 LPA-Fab complex and (D) 2.3 Å LT3015 Fab in the absence of lipid. The Fab backbone atoms are traced with a ribbon and the lipids are shown as a ball and stick (A) or space filling diagram (B and C). All structures are shown from the same view and the heavy and light chains are labeled in (A). In total, three three-dimensional structures of one Fab fragment were solved, one in the absence of lipid and two as Fab-lipid co-crystal complexes (14:0 LPA and 18:2 LPA). The electron density for the bound lipids is nearly complete, so the conformation of the bound lipids within the antigen-binding site is clearly defined (FIG. 2). In FIG. 2, the electron density (blue) from 2|FO|−|FC| refined maps contoured at (A) 1.1 s for the S1P-Fab complex, (B) 1.2 s for the 14:0 LPA-Fab complex and (C) 1.0 s for the 18:2 LPA-Fab complex.

The variable CDR loops at the antigen binding site adopt nearly identical conformations in the free and antigen-bound crystal structures. In the free LT3015 crystal, an elongated loop CDR-L1 adopts an extended conformation that stretches into solvent while CDR-H3 folds down over a vast hydrophobic cavity in a manner reminiscent of the homologous region of the LT1009 anti-S1P antibody (Wojciak, J. M. et al., Proc Natl Acad Sci USA. 2009 Oct. 20; 106(42):17717-22. Epub 2009 Oct. 7). Electron density for two sulfate ions is observable at the antigen binding site within the LT3015 Fab crystal structure. One ion resides at the interface between the heavy and light chains while the second is more surface-exposed and mediates contacts with a close-packed neighbor in the crystal. The presence of sulfate in the x-ray structure is not surprising as the crystal grew after equilibration against 1.75 M ammonium sulfate. Calculation and modeling of the electrostatic surface potential on the LT3015 Fab crystal structure reveals strongly electropositive pockets at both the sites where the two sulfates are bound (FIG. 2D). Another strong peak of elongated electron density was refined as triethylene glycol (PGE). As PGE this was not component of crystallization or crystal stabilization solutions it is likely that this represents a relatively short, ordered portion of a larger polyethylene glycol (PEG) polymer. Additive amounts of PEG 400 were necessary for LT3015 Fab crystallization.

LT3015 Fab:LPA (C14:0) complex crystal structure. In order to directly observe the interactions that mediate specific and high affinity binding of LPA by LT3015, the 1.98 Å x-ray crystal structure of the LT3015 Fab was determined after crystallization in complex with its LPA antigen. The version of LPA used in this experiment contained the fully saturated 14-carbon myristic acid (14:0) esterified to glycerol at the carbon-1 position. The complex crystallized in a face-centered orthorhombic space group with two complexes in the asymmetric unit allowing for independent refinement of two LT3015 Fab:LPA complex structures in slightly different chemical environments. The complex crystal structure reveals that LT3015 binds LPA through an intricate network hydrogen bonds and hydrophobic interactions involving amino acids from loops in both the light and heavy chains. The refined electron density is excellent for LPA at the antigen binding site in both complexes in the asymmetric unit.

LT3015 Fab:LPA (C18:2) complex crystal structure. The LPA (14:0) isotype is not commonly found in biological samples and is likely not as potent a signaling molecule as LPA that contains longer chain fatty acids with varying degrees of unsaturation. One of the most abundant and potent LPA isotypes contains linoleic acid, the 18-carbon di-unsaturated (18:2) fatty acid, esterified to glycerol at carbon-1. In order to determine how LT3015 binds LPA (18:2) the x-ray crystal structure of the LT3015 Fab:LPA (18:2) complex was solved after crystallization of the Fab: lipid complex. The complex was prepared in a manner similar to the LPA (14:0) complex and crystallized under similar conditions in the same space group. The structure reveals only minor differences between the two complex structures. Electron density about the LPA (18:2) ligand is not as well defined as for the saturated version (FIG. 2B, C). This is particularly noticeable in the region of the hydrocarbon tail where slightly higher values for thermal factors were generated during B factor refinement. Interestingly, the terminal portion (ω1-ω5 carbons) of LPA (18:2) hovers over an intramolecular hydrogen bond formed between the side chains of AsnH52 and SerH54. This interaction, along with the position of the TyrH98 side chain, projects the terminal portion of the hydrophobic tail away from the antibody and out into solvent. Otherwise, the antibody:antigen interactions observed in the LPA (14:0) are preserved in the structure of the complex between LT3015 and the unsaturated LPA (18:2).

Formation of the complex between the LT3015 Fab and its LPA antigen buries between 60 (18:2) and 67% (14:0) of the LPA molecular surface area. For LPA (18:2), this places it squarely in line with other small molecule antigens whose structures in complex with antibodies are known. LPA (14:0), on the other hand, is among the top small molecule antigens when ranked based on the ratio of percent buried surface/molecular surface area. The extent to which the LPA (14:0) is shielded from solvent by LT3015 is similar to that observed in the complex of the LT1009 antibody Fab fragment and its S1P antigen. Wojciak, J. M. et al., PNAS 2009, above. However, the nature of the interactions in the two antibody:antigen complexes differ drastically. LT1009 effectively buries the hydrocarbon tail portion of S1P while contacting its head group through two bridging Ca²⁺ ions that leave the phosphate relatively solvent exposed. In contrast, LT3015 almost completely buries the LPA phosphoglycerol head group while leaving significant portions of the hydrocarbon tail uncovered (FIGS. 1, 2).

Overall, the conformation of the LT3015 antibody is the same with or without antigen bound. Elbow angles for the two Fab:LPA complexes are 167.5° and 166.9° compared to 165.0° for the free Fab. Interestingly, the CDR loop conformations are also nearly perfectly shared by the Fab in its LPA complex and free crystal structures. Superposition of the free Fab on to the two Fab:LPA complexes leads to a root-mean-squared deviation (RMSD) of only 0.84 Å for all Ca positions. Remarkably, the value of this difference is precisely the same whether the RMSD is calculated for Ca positions throughout the entire Fab fragment, throughout the two variable domains only, or for just the six CDR loops.

Lipid specificity and cross-reactivity. In vitro competition binding assays were carried out in which a broad range of phospholipids lipids compete with biotinylated LPA (18:0) for binding to LT3015. The experiment revealed that neither the closely related sphingosine-1-phosphate (S1P) nor the choline-containing lysophophatidylcholine (LPC) lipids effectively compete with LPA for LT3015 binding. The LT3015 Fab:LPA crystal structures suggest that the unique amino alcohol head group of S1P would disrupt the LT3015 hydrogen bonding network and that the addition of choline would interfere with the close packing between the LT3015 light chain CDR loops, the GlyH100-SerH100A-GlyH100B hairpin turn from loop CDR-H3, and the lipid phosphate. Furthermore, as illustrated by the lipids phosphatidic acid (PA) and phosphatidylcholine (PC), the addition of fatty acids at the sn-2 position of glycerol disrupt LT3015 binding. This substitution likely disrupts the ability of LT3015 loop CDR-H3 to adopt the conformation observed in the LT3015 crystal structures. A combination of these factors likely contributes to the inability of LT3015 to bind the potent signaling lipid platelet-activating factor (PAF).

The strict specificity of LT3015 is further borne out upon comparison of its behavior in competition assays with lipids that are more closely structurally related to LPA. Alteration of the linkage between the fatty acid and glycerol from ester to ether yields lysophosphatidal acid (C18:1 ether LPA) and removal of LPA phosphate converts it to monoacyl glycerol (18:1 sn-1 ester MG). Neither of these subtle changes generates lipids with LT3015 binding affinities that are comparable to LPA. The only lipid tested by this assay that bound even weakly to the LT3015 antibody was cyclic phosphodiester version of LPA (18:1 ester cLPA) in which one phosphate moiety is covalently attached to both glycerol carbons-3 and -2, which fixes the orientation of the phosphate relative to the rest of the head group.

The structure of the Fab fragments from LT3015 was compared to that of the Fab fragment of the S1P monoclonal antibody, LT1009, described previously in U.S. patent application Ser. No. 12/631,784, filed 4 Dec. 2009, (LPT-4000-UT), the contents of which are incorporated herein in their entirety. Despite the structural homology of the Fab fragments and their lipid targets, the mechanisms of S1P and LPA recognition differ dramatically. Two calcium atoms bridge the phosphate group of S1P and a metal-coordinating side chains protruding from the light chain CDRL1 and L3. The amino alcohol head group of the sphingosine backbone is recognized through hydrogen bonding interactions from the side chain of Lys L50 (CDRL2) and the polypeptide backbone atoms of the antibody light and heavy chains. The S1P hydrophobic tail is almost completely enclosed within a hydrophobic channel formed primarily by the heavy chain.

In contrast, intermolecular metal bridging is not present in the structures of Fab-LPA complexes. The phosphate group of LPA hydrogen bonds to amino acid side chains in CDR L1, L2 and H3. Each oxygen atom forms simultaneous interactions with both oxygen and nitrogen atoms extending from the Fab scaffolding. The sn-2 hydroxyl of LPA forms a hydrogen bond with the backbone amide of Gly H99, and the C1 carbonyl in the LPA fatty acid moiety forms a hydrogen bond with Ser L91. The hydrocarbon tail is stabilized through hydrophobic interactions with the heavy chain of LT3015. All the intermolecular interactions present in the Fab-S1P and Fab-LPA complex structures are summarized in Table 22 and Table 23.

LT3015 employs eight amino acids from both its light and heavy chains to bind the LPA glycerophosphate head group through an intricate hydrogen bonding network. The AsnL30 and TyrL32 side chains from CDR-L1 each contribute one hydrogen atom to the same phosphate oxygen on LPA. Furthermore, additional amino acids from the extended CDR-L1 pack closely to the heavy chain nearly completely occluding the phosphate from solvent and likely increasing the stability of the buried hydrogen bonds. LysL50 from CDR-L2 participates in a hydrogen bond with phosphate while SerL91 from CDR-L3 shares a hydrogen with the acyl oxygen of the esterified myristic acid moiety. The only hydrogen bond mediated by an amino acid side chain from the antibody heavy chain is between TyrH100D from CDR-H3 and phosphate. The three remaining hydrogen bonds rely upon amide nitrogens from CDR-H3 glycine residues. GlyH100 and GlyH100B from CDR-H3 each contact phosphate oxygens while GlyH97 hydrogen bonds with the hydroxyl group at the carbon-2 position of glycerol. Two ordered water molecules occupy positions over the only surface of the LPA phosphate moiety that is accessible to solvent bringing the total to ten hydrogen bonds to LPA in the LT3015 Fab:LPA complex crystal structure.

The remaining contacts between LT3015 antibody and its LPA antigen are dominated by closely packed hydrophobic interactions. The LPA aliphatic tail is contacted by PheL94 and PheL96 from CDR-L3 as well as LeuH33 (CDR-H1), TyrH56 (CDR-H2), and PheH96, TyrH98, and TyrH99 (CDR-H3). TyrH99 passes over the glycerol portion of LPA and appears to stabilize the CDR-H3 conformation by fastening to the light chain through interactions with the side chains of H isL27D and PheL96. A similar “restraint” role is played by the identical TyrH98 amino acid in the anti-S1P LT1009 Fab:S1P complex crystal structure, suggesting that this interaction may be a conserved mechanism for stabilizing antibody-lipid complexes (Huxford et al.,).

TABLE 22
Intermolecular interaction in the structure of the S1P-Fab complex

| Residue | Position | Interactions with calcium or S1P |
|---|---|---|
| Asp | L30 | calcium coordination |
| Asp | L31 | calcium coordination |
| Asp | L32 | calcium coordination |
| Glu | L50 | Electrostatic contact with C₂ ammonium group |
| Asp | L92 | calcium coordination |
| Leu | L94 | contacts with C₈, C₁₀, C₁₂ |
| Phe | L96 | contacts with C₆, C₈ |
| Thr | H33 | contacts with C₁₃, C₁₄ |
| His | H35 | contacts with C₁₁, C₁₂, C₁₃ |
| Ala | H50 | contacts with C₁₂, C₁₄ |
| Ser | H52 | contacts with C₁₆, C₁₇, C₁₈ |
| His | H54 | contact with C₁₈ |
| Ile | H56 | contacts with C₁₅, C₁₇ |
| Lys | H58 | contacts with C₁₄, C₁₅ |
| Phe | H97 | contacts with C₉, C₁₀, C₁₁, C₁₃, C₁₆, C₁₈ |
| Tyr | H98 | contact with C₇, C₉, C₁₀, C₁₅, C₁₆, C₁₇ |
| Gly | H99 | Hydrogen bond (amide) with C₃ hydroxyl group |
| Ser | S100 | Hydrogen bond (carbonyl) with C₃ hydroxyl group |
| Thr | H100A | contact with C₁ |
| Trp | H100C | contacts with C₄, C₉, C₁₁ |

TABLE 23
Intermolecular interaction in the structure of the LPA-Fab complex

| Residue | Position | Interactions with 18:2 LPA |
|---|---|---|
| Asn | L30 | Hydrogen bond with phosphate oxygen |
| Tyr | L32 | Hydrogen bond with phosphate oxygen |
| Lys | L50 | Hydrogen bond with phosphate oxygen |
| Ser | L91 | Hydrogen bond with C₁ carbonyl oxygen |
| Phe | L94 | contact with C₄(FA) |
| Phe | L96 | contact with C₂, C₄ |
| Leu | H33 | contact with C₅, C₇ |
| Leu | H50 | contact with C₄, C₆, C₇ |
| Asn | H52 | Contacts C₁₀, C₁₈ |
| Ser | H54 | Contacts C₁₀, C₁₆, C₁₈ |
| Tyr | H56 | contact with C₈, C₉ |
| Phe | H96 | Contacts with Cₛₙ₋₁, Cₛₙ₋₂, Oₛₙ₋₁, Oₛₙ₋₂, C₁ carbonyl |
| Gly | H97 | Hydrogen bond (backbone amide) with Osn-2 |
| Tyr | H98 | contact with C₅, C₇, C₉, C₁₀, C₁₂, C₁₃, C₁₅ |
| Tyr | H99 | contact with C₂, C₃, C₄, C₅ |
| Gly | H100 | Hydrogen bond (backbone amide) with phosphate |
| | | oxygen |
| Gly | H100B | Hydrogen bond (backbone amide) with phosphate |
| | | oxygen |
| Tyr | H100D | Hydrogen bond with phosphate oxygen |

LPA binding by LT3015. In order to better understand the molecular mechanism by which LT3015 recognizes LPA antigens, LT3015 antibody whole IgG and Fab fragments were prepared and purified and tested for binding to different LPA isotypes in vitro. The two forms of the LT3015 antibody display comparable binding affinities toward a biotinylated stearic acid (18:0)-containing LPA. Neither whole IgG nor Fab fragment versions of the LT1009 antibody that recognizes the closely related biologically active lipid sphingosine-1-phosphate (S1P) interacts with LPA in this assay. LT3015 binding to two LPA isoforms containing either myristic acid (14:0) or linoleic acid (18:2) was next assayed based upon the ability of the native “unlabeled” LPA to compete with the biotinylated “labeled” LPA for binding to either the whole IgG or the isolated Fab fragment. This study yielded equilibrium inhibition constants (Kᵢ) that range between 50-250 nM. In general, both whole IgG and Fab preparations of LT3015 were found to bind with nanomolar affinity to diverse LPA isotypes in vitro.

Site-directed mutagenesis. Single point mutations were introduced in the pCON expression (Lonza Biologics) vectors containing the antibody heavy or light chains using the Quikchange site-directed mutagenesis kit (Stratagene), while heavy chain and light chain variants containing multiple mutations were with synthesized de novo (GeneArt) cloned into the pCON vectors. All plasmids were screened by DNA sequencing to confirm the presence of each mutation.

Production and purification of antibody variants. Plasmids harboring the desired mutations were amplified in E. coli and purified using the EndoFree maxi plasmid purification kit (Qiagen). The purified plasmids were transiently transfected at 0.5 μg/ml into the human embryonic kidney (HEK) cell line 293F using 293fectin and 293F-FreeStyle Media (Invitrogen). After 5 days in culture, the supernatants were harvested and diluted with equal volume IgG binding buffer (Pierce) and applied to a column packed with ProSep-vA-Ultra resin (Millipore) equilibrated with binding buffer. The bound IgG was washed with binding buffer, eluted with elution buffer (Pierce) and collected in fractions containing binding buffer (10% final volume) to neutralize the pH. IgG-containing fractions were concentrated using an Amicon stirred cell equipped with a 50 kDa MWCO filter (Millipore). The concentrated antibody was extensively dialyzed against 1×PBS (Cellgro) and filtered through a 0.22 mM syringe-driven filter unit (Millipore) and stored at 4° C.

Antibody optimization mutagenesis. Antibody positions that were deemed important for lipid binding based on the crystal structures were targeted for mutagenesis to identify variants with increased lipid-binding affinities. Full-length antibodies harboring single or multiple mutations in either the heavy or light chain or both were expressed, purified and assayed for binding with their bioactive lipid target using a direct ELISA or ligand titration ELISA (also called inverted ELISA).

Direct-binding ELISA. Microtiter ELISA plates (Costar) were coated by incubation with 18:0 LPA conjugated to delipidated BSA and diluted in 0.1M Na2CO3 (pH 9.5) at 37° C. for 1 hour. Plates were washed with PBS (137 mM NaCl, 2.68 mM KCl, 10.1 mM Na2HPO4, 1.76 mM KH2PO4 pH 7.4) and blocked with PBS/BSA/Tween-20 for 1 hour at room temperature or overnight at 4° C. Primary incubation of wild type or mutant full length antibodies (100 mL/well at the following concentrations: 0.4 mg/mL, 0.2 mg/mL, 0.1 mg/mL, 0.05 mg/mL, 0.0125 mg/mL, and 0 mg/mL) was carried out for 1 hour at room temperature. Plates were washed and incubated with 100 mL/well of HRP-conjugated goat anti-human diluted 1:50,000 (Jackson) for 1 hour at room temperature. After washing, peroxidase activity was measured with tetramethylbenzidine substrate (Sigma) and quenched by the addition of 1 M H2SO4. The optical density (OD) was measured at 450 nm using a Thermo Multiskan EX. The raw data was plotted using GraphPad software.

### Screening Humanized Anti-LPA Variants by Inverted ELISA

Materials: Jackson Immunoresearch gt anti-human IgG, Fcγ specific (Cat #109-005-098, 1.3 mg/ml stock, Lot #85916), LPA-PEG2-biotin, LP101200, 1 mM, SA-HPX, 1:30 K of 50% glycerol stock, (1:60K final dilution), LT3015, lot LR81055, 19.3 mg/ml; 5 μl, 96.5 μg plus 9 ml culture medium=10.7 μg/ml

Methods:

Synthesis of LPA-biotin The LPA-biotin conjugate was prepared by dissolving 15.3 mg of Maleimide-(PEG)₂-Biotin (Pierce Cat #21901) in 291 mL of DMSO (100 mM) and 2 mg of thiolated 18:0 LPA (Echelon) in 470 mL of DMSO (5 mM) followed by sonication for 10 minutes and vortex mixing for 1 minute. The conjugation reaction was performed in a 2-mL amber glass vial by combining 290 mL of each the biotin and LPA solutions and 870 mL of 2×PBS, which was made by diluting 10×PBS (Boston Bioproducts Cat #BM-220) 1:5 with water. The reaction was rocked at room temperature for 4 hours and then stored at 4° Celsius.

Anti-human IgG was diluted to 1 μg/ml: 30 μl/30 ml carbonate coating buffer, 50 μl/well Greiner high-binding ELISA plates×5 plates

Incubated overnight, 4° C.

100 μl blocking buffer was added (PBS-BSA-Tween).

Incubated RT, 1 hr.

Washed plates×3 w PBS

Added 25 μl antibody culture supernatant per well

Incubated RT, 1 hr

Washed plates×3 w PBS

Added 25 μl LPA-biotin dilution per well

Incubated RT, 1.5 hr

Washed plates×3 w PBS

Added 25 μl SA-HPX dilution per well

Incubated RT, 15 min

Washed plates×3 w PBS

Added 25 μl cold TMB

Incubated RT, 5′

Stopped color reaction with 25 μl of 1M H2S04 (½ of 2M stock)

Read at 450 nm.

LPA-Biotin Dilutions

1 mM stock, 5 μl plus 5 ml blocking buffer=1 μM.

1 μM, 4 ml plus 6 ml blocking buffer=400 nM

Placed 1 ml/well in column A of deep well plate

Used 0.5 ml plus 0.5 ml blocking buffer to make 2× dilutions across plate to column 11.

Column 12, “0” is buffer alone

SA-HPX Dilution

5 μl glycerol stock plus 5 ml blocking buffer=1/1000

1/1000, 0.5 ml plus 14.5 ml blocking buffer=1/30,000 of stock

The dissociation constant (KD) of LPA binding to the wild-type anti-LPA antibody measured 10 nM. In preliminary experiments, a few anti-LPA variants (Table 24) were evaluated in a ligand titration ELISA as described below.

TABLE 24
LPA binding of LT3015 and variants as determined by
ligand titration ELISA

| | | K_{D} | | |
|---|---|---|---|---|
| V_{L} plasmid | V_{H} plasmid | (nM LPA- | Bₘₐₓ | |
| (mutation) | (mutation) | biotin) | (A₄₅₀) | Binding |
| Heavy chain mutations | | | | |
| pATH502 | pATH602 | 10.6 | 1.15 | +++ |
| (WT) | (WT) | | | |
| pATH502 | pATH610 | — | — | − |
| (WT) | (Y99A) | | | |
| pATH502 | pATH611 | — | — | − |
| (WT) | (Y100dN) | | | |
| pATH502 | pATH628 | 11.4 | 1.16 | +++ |
| (WT) | (N52Y) | | | |
| pATH502 | pATH629 | 6.5 | 1.23 | ++++ |
| (WT) | (S54Y) | | | |
| pATH502 | pATH630 | 6.5 | 1.38 | ++++ |
| (WT) | (N52Y, S54Y) | | | |
| pATH502 | pATH631 | 14.9 | 1.16 | ++ |
| (WT) | (I30Y, N31Y, N52A, | | | |
| | P52aG, S54Y, K73Y) | | | |
| Light chain mutations | | | | |
| pATH510 | pATH602 | 6.8 | 1.37 | ++++ |
| (N30R) | (WT) | | | |
| pATH511 | pATH602 | — | — | − |
| (Y32R) | (WT) | | | |

A modest improvement in LPA binding (−2-fold) was observed for two heavy chain variants, pATH629 and pATH630, both of which contain an S54Y substitution in the heavy chain, and for a light chain variant, pATH510, which has an N30R substitution in the light chain. Mutations at certain other positions tested abrogated binding (pATH610, having a Y99A mutation in the heavy chain, and pATH611, having a Y100dN mutation in the heavy chain, as well as pATH511, having a Y32R mutation in the light chain. Additional studies are needed to optimize the anti-LPA antibody for improved antigen affinity. However, positions S54, Y99 and Y100d in the heavy chain and positions N30 and Y32 in the light chain appear to be important for antibody-LPA binding.

Affinity maturation using the anti-LPA crystal structures. Based upon the LT3015 Fab:LPA complex crystal structures, additional mutations were introduced at two positions in the antibody that contact either the phosphate group (AsnL30) or the terminal end of the fatty acid tail (AsnH52 and SerH54). AsnL30 was mutated to Arg based upon the assumption that the longer, basic amino acid side chain could better contact the LPA phosphate. AsnH52 and SerH54 were both mutated to Tyr in an effort to augment interactions between LT3015 and the hydrocarbon tail of LPA. When introduced separately, neither mutated antibody exhibited more than twofold alteration of its binding affinity for biotinylated LPA (18:0) as shown in the table above. However, the introduction of the mutations at both sites results in a mutated LT3015 antibody with significantly (roughly 5-fold) improved LPA binding affinity.

All of the compositions and methods described and claimed herein can be made and used without undue experimentation in light of the present disclosure. While these compositions and methods are described in terms of preferred embodiments, variations will be apparent to those of skill in the art, and all such similar substitutes and modifications are deemed to be within the spirit and scope of the invention as defined by the appended claims.

All patents, patent applications, and publications mentioned in the specification are indicative of the levels of skill of those of ordinary skill in the art to which the invention pertains. All patents, patent applications, and publications, including those to which priority or another benefit is claimed, are herein incorporated by reference to the same extent as if each individual publication was specifically and individually indicated to be incorporated by reference.

The invention illustratively described herein suitably can be practiced in the absence of any element(s) not specifically disclosed herein. Thus, for example, in each instance herein any of the terms “comprising”, “consisting essentially of”, and “consisting of” may be replaced with either of the other two terms. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed.

What is claimed is:

1. A crystalline composition comprising an anti-lysophosphatidic acid antibody or fragment thereof.

2. A crystalline composition according to claim 1 that further comprises a lysophosphatidic acid.

3. The crystalline composition of claim 1 wherein the anti-lysophosphatidic acid antibody or fragment thereof is a monoclonal antibody.

4. The crystalline composition of claim 1 wherein the anti-lysophosphatidic acid antibody or fragment thereof is a Fab fragment.

5. Use of a crystalline composition according to claim 1 in:
a. determining the structure of the anti-lysophosphatidic acid antibody or fragment thereof;
b. determining the ligand-binding characteristics of the anti-lysophosphatidic acid antibody or fragment thereof, or of a variant or fragment of variant of the antibody;
c. designing an antibody, or fragment thereof, specifically reactive with lysophosphatidic acid; and/or
d. optimizing or altering the affinity of a monoclonal antibody for lysophosphatidic acid.

6. The use of claim 5 wherein the use further includes deriving structural information about the crystalline composition.

7. A method of designing an optimized antibody to a lipid comprising:
a. providing an amino acid sequence of at least one variable region of a heavy or light chain of a first humanized anti-lipid antibody which is a humanized anti-lysophosphatidic acid antibody, wherein optionally at least one complementarity-determining region within the variable region is identified;
b. replacing or inserting one or more amino acids within the at least one variable region to yield a variant amino acid sequence, wherein the amino acid replacement or insertion is within a complementarity-determining region;
c. preparing a second humanized anti-lipid antibody containing the variant amino acid sequence, wherein the amino acid sequences of the first and second humanized anti-lipid antibodies differ only in the variant amino acid sequence;
d. determining one or more activity criteria of the second humanized antibody; and
e. selecting an optimized antibody based on one or more of the activity criteria, wherein the optimized antibody is the second humanized antibody.

8. The method of claim 7 which is performed in silico.

9. The method of claim 7 wherein determining one or more activity criteria of the second humanized antibody is by molecular modeling, ELISA or surface plasmon resonance.

10. The method of claim 7 wherein at least one of the activity criteria is binding affinity for lysophosphatidic acid, binding affinity for a second lipid, or specificity for lysophosphatidic acid or specificity for a second lipid.

11. The method of claim 7 wherein the second lipid is not lysophosphatidic acid.

12. A method according to claim 7 further comprising use of three-dimensional structural information about the binding of the anti-lysophosphatidic acid antibody and lysophosphatidic acid to select a location and/or identity of the amino acid replacement(s).

13. The method of claim 12 wherein the three-dimensional structural information is molecular modeling data or x-ray crystallography data.

14. An optimized antibody made according to claim 7.

15. A method according to claim 7 wherein the first humanized anti-lipid antibody is LT3015.

16. A method according to claim 7 wherein the one or more amino acids replaced is selected from the group consisting of:
asparagine at position 30 of the light chain; tyrosine at position 32 of the light chain; lysine at position 50 of the light chain, serine at position 91 of the light chain, phenylalanine at position 94 of the light chain, phenylalanine at position 96 of the light chain, leucine at position 33 of the heavy chain, leucine at position 50 of the heavy chain, asparagine at position 52 of the heavy chain, serine at position 54 of the heavy chain, tyrosine at position 56 of the heavy chain, phenylalanine at position 96 of the heavy chain, glycine at position 97 of the heavy chain, tyrosine at position 98 of the heavy chain, tyrosine at position 99 of the heavy chain, glycine at position 100 of the heavy chain, glycine at position 100B of the heavy chain and tyrosine at position 100D of the heavy chain.

17. A method according to claim 16 wherein the one or more amino acids replaced is selected from the group consisting of: asparagine at position 30 of the light chain, tyrosine at position 32 of the light chain; asparagine at position 52 of the heavy chain, serine at position 54 of the heavy chain, tyrosine at position 99 of the heavy chain and tyrosine at position 100D of the heavy chain.

18. A humanized anti-lysophospholipid antibody which is a variant of LT3015 having a substitution of one or more amino acids, said substitution being at a position selected from the group consisting of: asparagine at position 30 of the light chain; tyrosine at position 32 of the light chain; lysine at position 50 of the light chain, serine at position 91 of the light chain, phenylalanine at position 94 of the light chain, phenylalanine at position 96 of the light chain, leucine at position 33 of the heavy chain, leucine at position 50 of the heavy chain, asparagine at position 52 of the heavy chain, serine at position 54 of the heavy chain, tyrosine at position 56 of the heavy chain, phenylalanine at position 96 of the heavy chain, glycine at position 97 of the heavy chain, tyrosine at position 98 of the heavy chain, tyrosine at position 99 of the heavy chain, glycine at position 100 of the heavy chain, glycine at position 100B of the heavy chain and tyrosine at position 100D of the heavy chain.

19. A humanized anti-lysophospholipid antibody of claim 18 having a substitution of one or more amino acids, said substitution being at a position selected from the group consisting of: position 30 in the light chain, position 32 in the light chain, position 52 in the heavy chain, position 54 in the heavy chain, position 99A in the heavy chain and position 100D in the heavy chain.

20. A humanized anti-lysophospholipid antibody which is a variant of LT3015 having a substitution selected from the group consisting of an N30R mutation in the light chain, a Y32 mutation in the light chain, an N52Y mutation in the heavy chain, an S54Y mutation in the heavy chain, a Y99A mutation in the heavy chain and a Y100dN mutation in the heavy chain.


SEQUENCE LISTING (39 sequences)

SEQ ID NO: 1 - protein, 10 aa, Mus musculus
GYGFINYLIE

SEQ ID NO: 2 - protein, 17 aa, Mus musculus
LINPGSDYTNYNENFKG

SEQ ID NO: 3 - protein, 13 aa, Mus musculus
RFGYYGSGNYFDY

SEQ ID NO: 4 - protein, 16 aa, Mus musculus
TSGQSLVHINGNTYLH

SEQ ID NO: 5 - protein, 7 aa, Mus musculus
KVSNLFS

SEQ ID NO: 6 - protein, 9 aa, Mus musculus
SQSTHFPFT

SEQ ID NO: 7 - protein, 5 aa, Mus musculus
NYLIE

SEQ ID NO: 8 - protein, 122 aa, Mus musculus
QVQLQQSGAELVRPGTSVKVSCKASGYGFINYLIEWIKQRPGQGLEWIGLINPGSDYTNYNENFKGKATLTADKSSSTAYMHLSSLTSEDSAVYFCARRFGYYGSGNYFDYWGQGTTLTVSS

SEQ ID NO: 9 - protein, 112 aa, Mus musculus
DVVMTQTPLSLPVSLGDQASISCTSGQSLVHINGNTYLHWYLQKPGQSPKLLIYKVSNLFSGVPDRFSGSGSGTDFTLKISRVEAEDLGVYFCSQSTHFPFTFGTGTKLEIK

SEQ ID NO: 10 - protein, 112 aa, Mus musculus; identical to SEQ ID NO: 9

SEQ ID NO: 11 - protein, 112 aa, Artificial Sequence
  Humanized antibody sequence
DIVMTQTPLSLPVTPGEPASISCTSGQSLVHINGNTYLHWYLQKPGQSPQLLIYKVSNLFSGVPDRFSGSGSGTDFTLKISRVEAEDVGVYYCSQSTHFPFTFGQGTKLEIK

SEQ ID NO: 12 - protein, 112 aa, Artificial Sequence
  Humanized antibody sequence
DVVMTQTPLSLPVTPGEPASISCTSGQSLVHINGNTYLHWYLQKPGQSPKLLIYKVSNLFSGVPDRFSGSGSGTDFTLKISRVEAEDVGVYFCSQSTHFPFTFGQGTKLEIK

SEQ ID NO: 13 - protein, 112 aa, Artificial Sequence
  Humanized antibody sequence
DIVMTQTPLSLPVTPGEPASISCTSGQSLVHINGNTYLHWYLQKPGQSPKLLIYKVSNLFSGVPDRFSGSGSGTDFTLKISRVEAEDVGVYFCSQSTHFPFTFGQGTKLEIK

SEQ ID NO: 14 - protein, 112 aa, Artificial Sequence
  Humanized antibody sequence
DVVMTQTPLSLPVTPGEPASISCTSGQSLVHINGNTYLHWYLQKPGQSPQLLIYKVSNLFSGVPDRFSGSGSGTDFTLKISRVEAEDVGVYFCSQSTHFPFTFGQGTKLEIK

SEQ ID NO: 15 - protein, 112 aa, Artificial Sequence
  Humanized antibody sequence
DVVMTQTPLSLPVTPGEPASISCTSGQSLVHINGNTYLHWYLQKPGQSPKLLIYKVSNLFSGVPDRFSGSGSGTDFTLKISRVEAEDVGVYYCSQSTHFPFTFGQGTKLEIK

SEQ ID NO: 16 - protein, 112 aa, Artificial Sequence
  Humanized antibody sequence
DVVMTQTPLSLPVTPGEPASISCTSGQSLVHINGNTYLHWYLQKPGQSPQLLIYKVSNLFSGVPDRFSGSGSGTDFTLKISRVEAEDVGVYYCSQSTHFPFTFGQGTKLEIK

SEQ ID NO: 17 - protein, 112 aa, Artificial Sequence
  Humanized antibody sequence
DVVMTQTPLSLPVTPGEPASISCRSSQSLLKTNGNTYLHWYLQKPGQSPKLLIYKVSNRFSGVPDRFSGSGSGTDFTLKISRVEAEDVGVYFCSQSTHFPFTFGQGTKLEIK

SEQ ID NO: 18 - protein, 112 aa, Artificial Sequence
  Humanized antibody sequence
DVVMTQTPLSLPVTPGEPASISCRSSQSLLKTNGNTYLHWYLQKPGQSPQLLIYKVSNRFSGVPDRFSGSGSGTDFTLKISRVEAEDVGVYYCSQSTHFPFTFGQGTKLEIK

SEQ ID NO: 19 - protein, 112 aa, Artificial Sequence
  Humanized antibody sequence
DVVMTQTPLSLPVTPGEPASISCRSSQSLLKTNGNTYLHWYLQKPGQSPKLLIFKVSNRFSGVPDRFSGSGSGTDFTLKISRVEAEDVGVYFCSQSTHFPFTFGQGTKLEIK

SEQ ID NO: 20 - protein, 122 aa, Mus musculus; identical to SEQ ID NO: 8

SEQ ID NO: 21 - protein, 122 aa, Artificial Sequence
  Humanized antibody sequence
EVQLVQSGAEVKKPGESLKISCQSFGYIFINYLIEWVRQMPGQGLEWMGLINPGSDYTNYNENFKGQVTISADKSSSTAYLQWSSLKASDTAMYFCARRFGYYGSGNYFDYWGQGTMVTVSS

SEQ ID NO: 22 - protein, 122 aa, Artificial Sequence
  Humanized antibody sequence
EVQLVQSGAEVKKPGESLKISCQSFGYGFINYLIEWIRQMPGQGLEWIGLINPGSDYTNYNENFKGQATLSADKSSSTAYLQWSSLKASDTAMYFCARRFGYYGSGNYFDYWGQGTMVTVSS

SEQ ID NO: 23 - protein, 122 aa, Artificial Sequence; identical to SEQ ID NO: 22
  Humanized antibody sequence

SEQ ID NO: 24 - protein, 122 aa, Artificial Sequence
  Humanized antibody sequence
EVQLVQSGAEVKKPGESLKISCQAFGYIFINYLIEWIRQMPGQGLEWIGLINPGSDYTNYNENFKGQATLSADKSSSTAYLQWSSLKASDTAMYFCARRFGYYGSGNYFDYWGQGTMVTVSS

SEQ ID NO: 25 - protein, 122 aa, Artificial Sequence
  Humanized antibody sequence
EVQLVQSGAEVKKPGESLKISCQAFGYGFINYLIEWVRQMPGQGLEWIGLINPGSDYTNYNENFKGQATLSADKSSSTAYLQWSSLKASDTAMYFCARRFGYYGSGNYFDYWGQGTMVTVSS

SEQ ID NO: 26 - protein, 122 aa, Artificial Sequence
  Humanized antibody sequence
EVQLVQSGAEVKKPGESLKISCQAFGYGFINYLIEWIRQMPGQGLEWMGLINPGSDYTNYNENFKGQATLSADKSSSTAYLQWSSLKASDTAMYFCARRFGYYGSGNYFDYWGQGTMVTVSS

SEQ ID NO: 27 - protein, 122 aa, Artificial Sequence
  Humanized antibody sequence
EVQLVQSGAEVKKPGESLKISCQAFGYGFINYLIEWIRQMPGQGLEWIGLINPGSDYTNYNENFKGQVTLSADKSSSTAYLQWSSLKASDTAMYFCARRFGYYGSGNYFDYWGQGTMVTVSS

SEQ ID NO: 28 - protein, 122 aa, Artificial Sequence
  Humanized antibody sequence
EVQLVQSGAEVKKPGESLKISCQAFGYGFINYLIEWIRQMPGQGLEWIGLINPGSDYTNYNENFKGQATISADKSSSTAYLQWSSLKASDTAMYFCARRFGYYGSGNYFDYWGQGTMVTVSS

SEQ ID NO: 29 - protein, 122 aa, Artificial Sequence
  Humanized antibody sequence
EVQLVQSGAEVKKPGESLKISCQAFGYGFINYLIEWVRQMPGQGLEWIGLINPGSDYTNYNENFKGQVTISADKSSSTAYLQWSSLKASDTAMYFCARRFGYYGSGNYFDYWGQGTMVTVSS

SEQ ID NO: 30 - protein, 122 aa, Artificial Sequence
  Humanized antibody sequence
EVQLVQSGAEVKKPGESLKISCQSFGYGFINYLIEWVRQMPGQGLEWIGLINPGSDYTNYNENFKGQATLSADKSSSTAYLQWSSLKASDTAMYFCARRFGYYGSGNYFDYWGQGTMVTVSS

SEQ ID NO: 31 - protein, 122 aa, Artificial Sequence
  Humanized antibody sequence
EVQLVQSGAEVKKPGESLKISCQAFGYGFINYLIEWIRQMPGQGLEWIGLINPGSDYTNYNENFKGQATLSADKSSSTAYLQWSSLKASDTAMYFCARRFGYYGSGNYFDYWGQGTMVTVSS

SEQ ID NO: 32 - protein, 122 aa, Artificial Sequence; identical to SEQ ID NO: 31
  Humanized antibody sequence

SEQ ID NO: 33 - protein, 122 aa, Artificial Sequence; identical to SEQ ID NO: 31
  Humanized antibody sequence

SEQ ID NO: 34 - protein, 122 aa, Artificial Sequence
  Humanized antibody sequence
EVQLVQSGAEVKKPGESLKISCQAFGYGFINYLIEWIRQMPGQGLEWIGAINPGSDYTNYNENFKGQATLSADKSSSTAYLQWSSLKASDTAMYFCARRFGYYGSGNYFDYWGQGTMVTVSS

SEQ ID NO: 35 - protein, 122 aa, Artificial Sequence
  Humanized antibody sequence
EVQLVQSGAEVKKPGESLKISCQAFGYGFINYLIEWIRQMPGQGLEWIGLIYPDSGYINYNENFKGQATLSADKSSSTAYLQWSSLKASDTAMYFCARRFAYYGSGYYFDYWGQGTMVTVSS

SEQ ID NO: 36 - protein, 122 aa, Artificial Sequence
  Humanized antibody sequence
EVQLVQSGAEVKKPGESLKISCQAFGYAFTNYLIEWVRQMPGQGLEWMGLIYPDSGYINYNENFKGQVTISADKSSSTAYLQWSSLKASDTAMYFCARRFAYYGSGYYFDYWGQGTMVTVSS

SEQ ID NO: 37 - protein, 122 aa, Artificial Sequence
  Humanized antibody sequence
EVQLVQSGAEVKKPGESLKISCQAFGYAFTNYLIEWVRQMPGQGLEWIGLIYPDSGYINYNENFKGQATLSADKSSSTAYLQWSSLKASDTAMYFCARRFAYYGSGYYFDYWGQGTMVTVSS

SEQ ID NO: 38 - protein, 122 aa, Artificial Sequence
  Humanized antibody sequence
EVQLVQSGAEVKKPGESLKISCQAFGDAFTNYLIEWVRQMPGQGLEWMGLIYPDSGYINYNENFKGQVTISADRSSSTAYLQWSSLKASDTAMYFCARRFAYYGSGYYFDYWGQGTMVTVSS

SEQ ID NO: 39 - protein, 122 aa, Artificial Sequence
  Humanized antibody sequence
EVQLVQSGAEVKKPGESLKISCQAFGDAFTNYLIEWVRQMPGQGLEWIGLIYPDSGYINYNENFKGQATLSADRSSSTAYLQWSSLKASDTAMYFCARRFAYYGSGYYFDYWGQGTMVTVSS